(19) United States
(12) Patent Application Publication
(10) Pub. No.: US 2026/0144799 A1
(43) Pub. Date: May 28, 2026

(54) COMPOSITION COMPRISING A FIRST RAS INHIBITOR, SECOND RAS INHIBITOR AND A SHP2 INHIBITOR FOR USE IN THE TREATMENT OF CANCER

(71) Applicant: Revolution Medicines, Inc., Redwood City, CA (US)

(72) Inventors: Cristina BLAJ, Oakland, CA (US); Steve KELSEY, Redwood City, CA (US); Elsa QUINTANA, Belmont, CA (US)

(21) Appl. No.: 19/120,279

(22) PCT Filed: Oct. 12, 2023
(86) PCT No.: PCT/US2023/035028

§ 371 Date: Apr. 11, 2025

Related U.S. Application Data
(60) Provisional application No. 63/415,635, filed on Oct. 12, 2022.

(51) Int. Cl. A61K 31/5386; A61K 31/497; A61K 31/504; A61K 45/06; A61P 35/00

(52) U.S. Cl. CPC A61K 31/5386; A61K 31/497; A61K 31/504; A61K 45/06; A61P 35/00

(57) ABSTRACT

The disclosure features methods for treating cancer (e.g., immune refractory cancer) using a RAS inhibitor. The disclosure also features combination therapies containing one or more RAS inhibitors for treating cancer.

### BACKGROUND

Cancer remains one of the most-deadly threats to human health. In the U.S., cancer affects nearly 1.3 million new patients each year, and is the second leading cause of death after heart disease, accounting for approximately 1 in 4 deaths.

The development of immune checkpoint inhibitors (ICIs) has dramatically improved the treatment of various solid tumors. However, initial or acquired resistance to treatment with ICIs remains an obstacle to a durable antitumor activity in most cases. Current response biomarkers for treatment with anti-PD-1 or anti-PD-L1 include tumor mutational burden, expression of Programmed cell Death Ligand-1 (PD-L1), and T cell density. The ICI induced anti-tumor immunity is dependent on the infiltration of lymphocytes into the tumor core, with “T cells inflamed” tumors showing the best responses. In contrast, “cold tumors,” also referred to as immune refractory or immune evasive tumors, which may be defined in part by a lack of T cell infiltration and a low IFN-g gene signature, rarely respond to immune checkpoint inhibition (Bonaventura et al, Front. Immunol. 2019). There is a need for new compositions, uses, and methods for treating immune refractory tumors.

### SUMMARY

The present disclosure provides compositions, uses, and methods thereof for treating cancer (e.g., immune refractory cancer, such as immune refractory lung cancer). The disclosure is based, at least in part, on the observation that immune refractory cancers can be treated with a combination of (i) a first RAS inhibitor, (ii) a second RAS inhibitor, and (iii) a SHP2 inhibitor, optionally in combination with (iv) an immune checkpoint inhibitor. In some embodiments the first RAS inhibitor inhibits RAS having an oncogenic G12C mutation. In some embodiments the RAS inhibitor is a covalent inhibitor, for example, capable of forming a covalent bond with an oncogenic mutant form of RAS G12C at the G12C position. In some embodiments, the second RAS inhibitor is a RAS(MULTI) inhibitor. In some embodiments treatment with a RAS inhibitor sensitizes the cancer to treatment with an immune checkpoint inhibitor or a SHP2 inhibitor. In some embodiments a compound or combination of compounds described herein are administered to a subject who has previously failed immunotherapy treatment, such as immunotherapy treatment with an immune checkpoint inhibitor.

In a first aspect, the disclosure provides a method of treating cancer in a subject. The method includes administering to the subject a) a first RAS inhibitor, or a pharmaceutically acceptable salt thereof; b) a second RAS inhibitor, or a pharmaceutically acceptable salt thereof; and c) a SHP2 inhibitor, or a pharmaceutically acceptable salt thereof. In some embodiments, the cancer is an immune refractory cancer.

In another aspect, the disclosure provides a use of a) a first RAS inhibitor, or a pharmaceutically acceptable salt thereof; b) a second RAS inhibitor, or a pharmaceutically acceptable salt thereof; and c) a SHP2 inhibitor, or a pharmaceutically acceptable salt thereof, in the treatment of cancer in a subject. In some embodiments, the cancer is an immune refractory cancer.

In another aspect, the disclosure provides a use of a) a first RAS inhibitor, or a pharmaceutically acceptable salt thereof; b) a second RAS inhibitor, or a pharmaceutically acceptable salt thereof; and c) a SHP2 inhibitor, or a pharmaceutically acceptable salt thereof in the manufacture of a medicament for treating cancer in a subject. In some embodiments, the cancer is an immune refractory cancer.

In another aspect, the disclosure provides a method of sensitizing an immune refractory lung cancer in a subject, the method including administering to the subject a combination of (i) a first RAS inhibitor, (ii) a second RAS inhibitor, and (iii) a SHP2 inhibitor, optionally in combination with (iv) an immune checkpoint inhibitor.

In another aspect, the disclosure provides a use of (i) a first RAS inhibitor, (ii) a second RAS inhibitor, and (iii) a SHP2 inhibitor, optionally in combination with (iv) an immune checkpoint inhibitor for sensitizing an immune refractory lung cancer in a subject.

In another aspect, the disclosure provides a use of (i) a first RAS inhibitor, (ii) a second RAS inhibitor, and (iii) a SHP2 inhibitor, optionally in combination with (iv) an immune checkpoint inhibitor in the manufacture of a medicament for sensitizing an immune refractory lung cancer in a subject.

In some embodiments of the methods or uses described herein, the subject has previously been administered an immune checkpoint inhibitor. In some embodiments, the subject is resistant to treatment with an immune checkpoint inhibitor. In some embodiments, the subject has acquired resistance to treatment with an immune checkpoint inhibitor.

In some embodiments, administering the first RAS inhibitor or the second RAS inhibitor sensitizes the cancer to treatment with an immune checkpoint inhibitor.

In some embodiments, the method or use further includes administering to the subject an immune checkpoint inhibitor. In some embodiments, the immune checkpoint inhibitor is a PD-1 inhibitor.

In some embodiments, the inhibitors are administered simultaneously or sequentially. In some embodiments, the inhibitors are administered as a single formulation or in separate formulations.

In some embodiments, the subject has one or more tumors with a low tumor mutational burden. In some embodiments, the subject has one or more microsatellite stable tumors. In some embodiments, the subject has one or more tumors with low microsatellite instability. In some embodiments, the subject has one or more tumors with a low tumor immune infiltrate.

In some embodiments, administering the combination of (i) a first RAS inhibitor, (ii) a second RAS inhibitor, (iii) a SHP2 inhibitor, and optionally (iv) an immune checkpoint inhibitor alters the tumor immune infiltrate. In some embodiments, the tumor immune infiltrate includes antigen-presenting cells, myeloid cells, or lymphoid cells. In some embodiments, administering the combination of (i) a first RAS inhibitor, (ii) a second RAS inhibitor, (iii) a SHP2 inhibitor, and optionally (iv) an immune checkpoint inhibitor alters the anti-tumor immune response. In some embodiments, administering the combination of (i) a first RAS inhibitor, (ii) a second RAS inhibitor, (iii) a SHP2 inhibitor, and optionally (iv) an immune checkpoint inhibitor alters the tumor microenvironment. In some embodiments, administering the combination of (i) a first RAS inhibitor, (ii) a second RAS inhibitor, (iii) a SHP2 inhibitor, and optionally (iv) an immune checkpoint inhibitor transforms an immunologically cold tumor into an immunologically hot tumor. In some embodiments, the method or use reduces tumor size or inhibits tumor growth.

In some embodiments, the first RAS inhibitor is a RAS G12C inhibitor. In some embodiments, the second RAS inhibitor is a RAS(MULTI) inhibitor. In some embodiments, the first RAS inhibitor is a RAS G12C inhibitor and the second RAS inhibitor is RAS(MULTI) inhibitor.

In some embodiments, the SHP2 inhibitor is RMC-4550, or a pharmaceutically acceptable salt thereof. In some embodiments, the SHP2 inhibitor is RMC-4630, or a pharmaceutically acceptable salt thereof.

In some embodiments, the second RAS inhibitor has the structure of Formula AI:

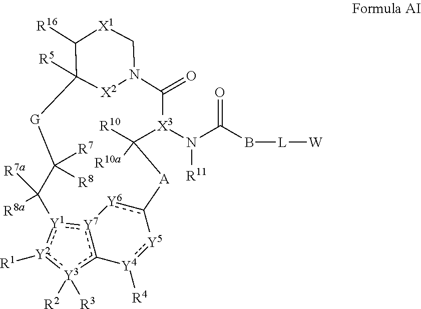

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is hydrogen, cyano, S(O)₂R′, optionally substituted amino, optionally substituted amido, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′} C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R¹⁶ is hydrogen or C₁-C₃ alkyl.

In some embodiments, the second RAS inhibitor is a compound selected from Table A1 or Table A2.

In some embodiments, the first RAS inhibitor has the structure of Formula BI:

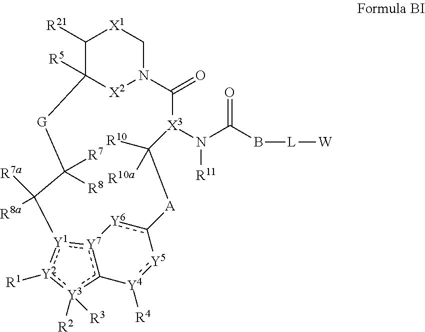

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′} C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl; or
R⁹ and R^{9′}, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R¹⁰ is hydrogen or halo;
R¹¹ is hydrogen or C₁-C₃ alkyl; and
R²¹ is hydrogen or C₁-C₃ alkyl (e.g., methyl).

In some embodiments, the first RAS inhibitor is a compound selected from Table B1 or Table B2.

In some embodiments, the second RAS inhibitor is a compound having the structure of Formula DIa:

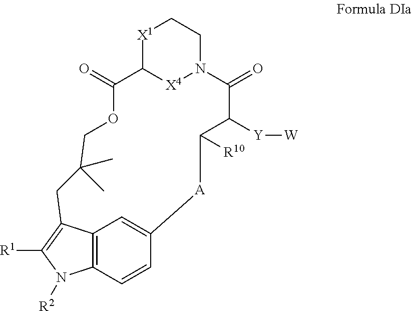

wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
Y is

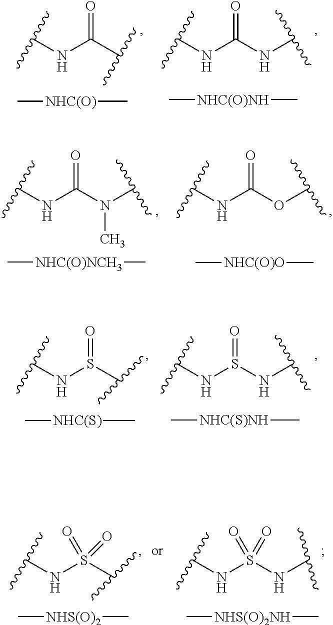

W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X¹ and X⁴ are each, independently, CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and R¹⁰ is hydrogen, hydroxy, optionally substituted C₁-C₃ alkyl, or optionally substituted C₁-C₆ heteroalkyl.

In some embodiments, the second RAS inhibitor is a compound selected from Table DIa, Table DIb, Table D2, or Table D3.

In some embodiments of the methods or uses described herein, the cancer is a lung cancer. In some embodiments, the cancer is an immune refractory lung cancer. In some embodiments, the immune-refractory lung cancer is a non-small cell lung cancer or a small-cell lung cancer. In some embodiments, the immune refractory lung cancer includes a Ras mutation. In some embodiments, the Ras mutation is K-Ras G12C, H-Ras C12C, or N-Ras G12C. In some embodiments, the Ras mutation is K-Ras G12C.

It is specifically contemplated that any limitation discussed with respect to one embodiment of the disclosure may apply to any other embodiment of the disclosure. Furthermore, any compound or composition of the disclosure may be used in any method or any use of the disclosure, and any method or any use of the disclosure may be used to produce or to utilize any compound or composition of the disclosure.

### BRIEF DESCRIPTION OF THE FIGURES

FIG. 1A-1G show the reduction in tumor volume over a 120 day period upon treatment with a vehicle control (FIG. 1A), Compound A (FIG. 1B), Compound B (FIG. 1C), RMC-4550 (FIG. 1D), Compound A in combination with RMC-4550 (FIG. 1E), Compound A in combination with Compound B (FIG. 1F), or a triple combination of Compound A, Compound B, and RMC-4550 (FIG. 1G).

FIG. 2A-2C shows that various combination therapies result in a decrease in myeloid derived suppressor cells (MDSCs) (FIG. 2A) as well as an increase in T cells (FIG. 2B) and an increase in T cell activity (FIG. 2C). In each of FIG. 2A-2C, each data point represents, from left to right, treatment with vehicle control, Compound A, Compound B, RMC-4550, Compound A in combination with RMC-4550, Compound A in combination with Compound B, and a triple combination of Compound A, Compound B, and RMC-4550, respectively.

### DETAILED DESCRIPTION

The present disclosure relates generally to compositions and methods for the treatment of cancer (e.g., immune refractory cancer). The disclosure is based, at least in part, on the observation that immune refractory cancer can be treated with a combination of (i) a first RAS inhibitor, (ii) a second RAS inhibitor, and (iii) a SHP2 inhibitor. In some embodiments the first RAS inhibitor inhibits RAS having an oncogenic G12C mutation. In some embodiments the first RAS inhibitor is a covalent inhibitor, for example, is capable of forming a covalent bond with an oncogenic mutant form of RAS G12C at the G12C position. In some embodiments the second RAS inhibitor is a RAS(MULTI) inhibitor. In some embodiments treatment with a RAS inhibitor sensitizes the cancer to treatment with an immune checkpoint inhibitor or a SHP2 inhibitor. In some embodiments a compound or combination of compounds described herein is administered to a subject who has previously failed immunotherapy treatment, such as treatment with an immune checkpoint inhibitor.

### General Methods

The practice of the present disclosure will employ, unless otherwise indicated, conventional techniques of cell culturing, molecular biology (including recombinant techniques), microbiology, cell biology, biochemistry, and immunology, which are within the skill of the art. Such techniques are explained fully in the literature, such as, Molecular Cloning: A Laboratory Manual, third edition (Sambrook et al., 2001) Cold Spring Harbor Press; Oligonucleotide Synthesis (P. Herdewijn, ed., 2004); Animal Cell Culture (R. I. Freshney), ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Manual of Clinical Laboratory Immunology (B. Detrick, N. R. Rose, and J. D. Folds eds., 2006); Immunochemical Protocols (J. Pound, ed., 2003); Lab Manual in Biochemistry: Immunology and Biotechnology (A. Nigam and A. Ayyagari, eds. 2007); Immunology Methods Manual: The Comprehensive Sourcebook of Techniques (Ivan Lefkovits, ed., 1996); Using Antibodies: A Laboratory Manual (E. Harlow and D. Lane, eds., 1988); and others.

### Definitions

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond.

A “compound of the present invention” and similar terms as used herein, whether explicitly noted or not, refers to Ras inhibitors described herein, including compounds of Formula I and subformula thereof, and compounds of Table 1 and Table 2, as well as salts (e.g., pharmaceutically acceptable salts), solvates, hydrates, stereoisomers (including atropisomers), and tautomers thereof.

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present disclosure include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present disclosure described herein, by substituting an isotopically labeled reagent for a non-isotopically labeled reagent.

Non-limiting examples of moieties that may contain one or more deuterium substitutions in compounds of the present invention, where any position “R” may be deuterium (D), include

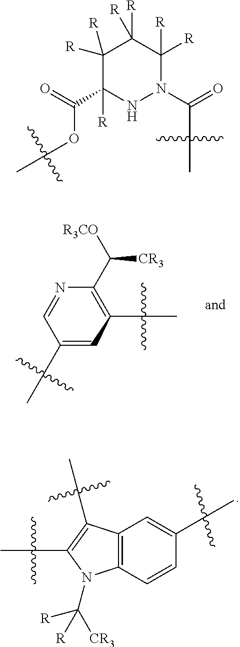

Additional examples include moieties such as

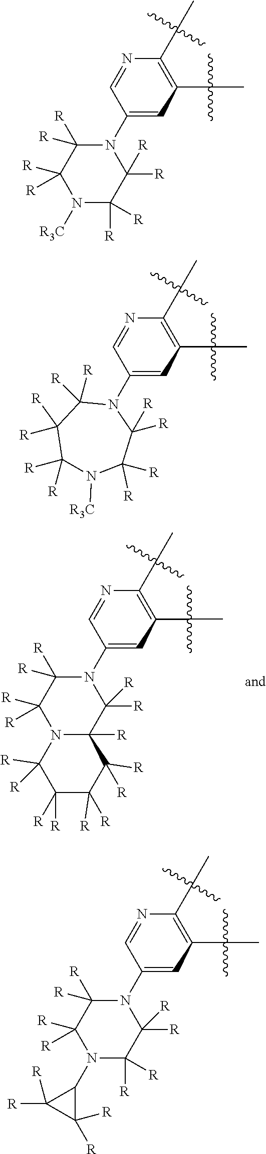

and deuteration of similar R¹-type moieties, wherein the definition of R¹ is found herein. Deuteration of moieties within substituent W in compounds of the present invention are also contemplated, where W is defined herein (see, e.g., Formulas I and subformulas thereof as well as specific examples of W described herein, such as

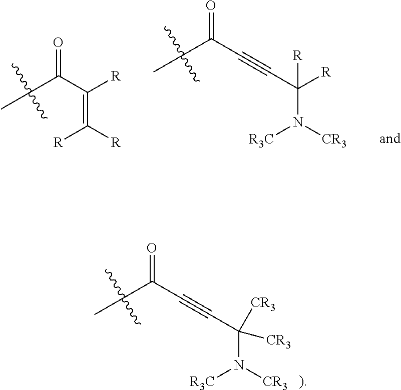

Moreover, deuteration of available positions in any A moiety of compounds of the Formulas described herein is also contemplated, such as

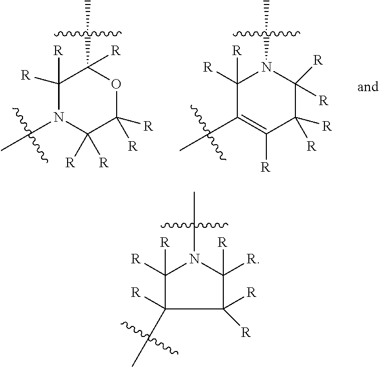

Further, deuterium substitution may also take place in compounds of the present invention at the linker position, such as

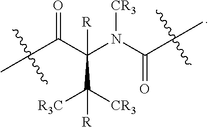

In a further embodiment, silylation substitution is also contemplated, such as in the linker as follows:

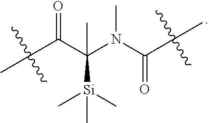 [SMILES: *C(=O)N(C)[C@@](C)(C(*)=O)[Si](C)(C)C]

As is known in the art, many chemical entities can adopt a variety of different solid forms such as, for example, amorphous forms or crystalline forms (e.g., polymorphs, hydrates, solvate). In some embodiments, compounds of the present disclosure may be utilized in any such form, including in any solid form. In some embodiments, compounds described or depicted herein may be provided or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium; halogen; —(CH₂)₀₋₄R^{∘}; —(CH₂)₀₋₄OR^{∘}; —O(CH₂)₀₋₄R^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; 4-8 membered saturated or unsaturated heterocycloalkyl (e.g., pyridyl); 3-8 membered saturated or unsaturated cycloalkyl (e.g., cyclopropyl, cyclobutyl, or cyclopentyl); —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘}) C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄—C(O)—N(R^{∘})₂; —(CH₂)₀₋₄—C(O)—N(R⁰)—S(O)₂—R^{∘}; —C(NCN)NR^{∘}₂; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NOR^{∘})NR^{∘}₂; —C(NH)NR^{∘}₂; —P(O)₂R^{∘}; —P(O)R^{∘}₂; —P(O)(OR^{∘})₂; —OP(O)R^{∘}₂; —OP(O)(OR^{∘})₂; —OP(O)(OR^{∘})R^{∘}, —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{●} may be substituted as defined below and is independently hydrogen, —C₁₋₆ aliphatic, —CH₂Ph, —O(CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{●} (or the ring formed by taking two independent occurrences of R^{●} together with their intervening atoms), may be, independently, halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, =NNHC(O)R*, =NNHC(O)OR*, =NNHS(O)₂R*, =NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH) NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on an aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{†} include ═O and ═S.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

As used herein, the term “administration” refers to the administration of a composition (e.g., a compound, or a preparation that includes a compound as described herein) to a subject or system. Administration also includes administering a prodrug derivative or analog of the compound or pharmaceutically acceptable salt of the compound or composition to the subject, which can form an equivalent amount of active compound within the subject's body. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal or vitreal.

The term “acetyl,” as used herein, refers to the group —C(O)CH₃.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cₓ-C_{y} alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “alkynyl sulfone,” as used herein, represents a group comprising the structure

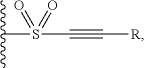

wherein R is any chemically feasible substituent described herein.

The term “amino,” as used herein, represents —N(R^{†})₂, e.g., —NH₂ and —N(CH₃)₂.

The term “aminoalkyl,” as used herein, represents an alkyl moiety substituted on one or more carbon atoms with one or more amino moieties.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in its broadest sense, refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. Exemplary amino acids include alanine, arginine, asparagine, aspartic acid, cysteine, glutamic acid, glutamine, glycine, histidine, optionally substituted hydroxylnorvaline, isoleucine, leucine, lysine, methionine, norvaline, ornithine, phenylalanine, proline, pyrrolysine, selenocysteine, serine, taurine, threonine, tryptophan, tyrosine, and valine.

An “amino acid substitution,” as used herein, refers to the substitution of a wild-type amino acid of a protein with a non-wild-type amino acid. Amino acid substitutions can result from genetic mutations and may alter one or more properties of the protein (e.g., may confer altered binding affinity or specificity, altered enzymatic activity, altered structure, or altered function).

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “C₀,” as used herein, represents a bond. For example, part of the term —N(C(O)—(C₀-C₅ alkylene-H)— includes —N(C(O)—(C₀ alkylene-H)—, which is also represented by —N(C(O)—H)—.

The terms “carbocyclic” and “carbocyclyl,” as used herein, refer to a monovalent, optionally substituted C₃-C₁₂ monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O)(OH), COOH, or C(O)OH or the unprotonated counterparts.

The term “combination therapy” refers to a method of treatment including administering to a subject at least two therapeutic agents, optionally as one or more pharmaceutical compositions, as part of a therapeutic regimen. For example, a combination therapy may include administration of a single pharmaceutical composition including at least two therapeutic agents and one or more pharmaceutically acceptable carrier, excipient, diluent, or surfactant. A combination therapy may include administration of two or more pharmaceutical compositions, each composition including one or more therapeutic agent and one or more pharmaceutically acceptable carrier, excipient, diluent, or surfactant. The two or more agents may optionally be administered simultaneously (as a single or as separate compositions) or sequentially (as separate compositions). The therapeutic agents may be administered in an effective amount. The therapeutic agent may be administered in a therapeutically effective amount. In some embodiments, the effective amount of one or more of the therapeutic agents may be lower when used in a combination therapy than the therapeutic amount of the same therapeutic agent when it is used as a monotherapy, e.g., due to an additive or synergistic effect of combining the two or more therapeutics.

The term “cyano,” as used herein, represents a —CN group.

The term “cycloalkyl,” as used herein, represents a monovalent saturated cyclic hydrocarbon group, which may be bridged, fused or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a monovalent, non-aromatic, saturated cyclic hydrocarbon group, which may be bridged, fused or spirocyclic having from three to eight ring carbons, unless otherwise specified, and containing one or more carbon-carbon double bonds.

The term “diastereomer,” as used herein, means stereoisomers that are not mirror images of one another and are non-superimposable on one another.

As used herein, the term “dosage form” refers to a physically discrete unit of a compound (e.g., a compound of the present disclosure) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., a compound of the present disclosure) has a recommended dosing regimen, which may involve one or more doses. In some embodiments, a dosing regimen includes a plurality of doses each of which are separated from one another by a time period of the same length; in some embodiments, a dosing regimen includes a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen includes a first dose in a first dose amount, followed by one or more additional doses in a second dose amount different from the first dose amount. In some embodiments, a dosing regimen includes a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

The term “disorder” is used in this disclosure to mean, and is used interchangeably with, the terms disease, condition, or illness, unless otherwise indicated.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “guanidinyl,” refers to a group having the structure:

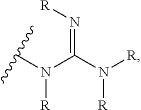

wherein each R is, independently, any any chemically feasible substituent described herein.

The term “guanidinoalkyl alkyl,” as used herein, represents an alkyl moiety substituted on one or more carbon atoms with one or more guanidinyl moieties.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N, or S atom). The heteroatom may appear in the middle or at the end of the radical.

The term “heteroaryl,” as used herein, represents a monovalent, monocyclic or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiment, the heteroaryl is substituted with 1, 2, 3, or 4 substituents groups.

The term “heterocycloalkyl,” as used herein, represents a monovalent monocyclic, bicyclic or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “hydroxy,” as used herein, represents a —OH group.

The term “hydroxyalkyl,” as used herein, represents an alkyl moiety substituted on one or more carbon atoms with one or more —OH moieties.

The term “isomer,” as used herein, means any tautomer, stereoisomer, atropiosmer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used interchangeably herein, the terms “immune refractory,” “immune evasive,” or “cold tumor” refers to a tumor, cancer, or patient having a tumor or cancer, for which a prior immunotherapy, such as an immune checkpoint inhibitor, has been found ineffective or intolerable. For example, a patient having an immune refractory cancer includes a patient who has previously been administered an immunotherapy, such as an immune checkpoint inhibitor, and the immunotherapy has been found ineffective or found not adequately effective to slow or halt the progression of the disease or to alleviate symptoms associated with the progression of the disease. Immune refractory cancers include cancers that have become resistant to or desensitized to treatment with immunotherapy (e.g., the effectiveness of an immunotherapy, such as an immune checkpoint inhibitor, previously administered to the patient is diminished overtime). Immune refractory cancers can be identified by methods known to those of skill in the art or by methods described herein. For example, immune refractory cancers may be characterized by low immune cell infiltrate in one or more tumors. Low immune cell infiltrate may include a decrease or absence of lymphocytes; a decrease or absence of tumor-infiltrating lymphocytes (TILs); a decrease or absence of dendritic cells; a decrease or absence of myeloid cells; a decrease or absence of natural killer (NK) cells; a decrease or absence of macrophages; a decrease or absence of T cells; a decrease or absence of CD8+ T cells; a decrease or absence of CD4+ T cells; or a decrease or absence of CD4+/CD8+ T cells. See, e.g., Chen and Mellman, Nature, 541:321 (2017). In some embodiments, By contrast, a “hot tumor”” refers to a tumor, cancer, or patient having a tumor or cancer that is not immune refractory. Cells having a low cytotoxic T cell count can be characterized as an “immune desert.” In some embodiments, cells having a cytotoxic T cell count of less than 1% live cells are considered an “immune desert.” In some embodiments, cells having a cytotoxic T cell count of less than 0.5% live cells are considered an “immune desert.” In some embodiments, cells having a cytotoxic T cell count of less than 0.25% live cells are considered an “immune desert.”

As used herein, the term “inhibitor” refers to a compound that prevents a biomolecule, (e.g., a protein, nucleic acid) from completing or initiating a reaction. An inhibitor can inhibit a reaction by competitive, uncompetitive, or non-competitive means, for example. With respect to its binding mechanism, an inhibitor may be an irreversible inhibitor or a reversible inhibitor. Exemplary inhibitors include, but are not limited to, nucleic acids, DNA, RNA, shRNA, siRNA, proteins, protein mimetics, peptides, peptidomimetics, antibodies, small molecules, chemicals, analogs that mimic the binding site of an enzyme, receptor, or other protein. In some embodiments, the inhibitor is a small molecule, e.g., a low molecular weight organic compound, e.g., an organic compound having a molecular weight (MW) of less than 1200 Daltons (Da). In some embodiments, the MW is less than 1100 Da. In some embodiments, the MW is less than 1000 Da. In some embodiments, the MW is less than 900 Da. In some embodiments, the range of the MW of the small molecule is between 800 Da and 1200 Da. Small molecule inhibitors include cyclic and acyclic compounds. Small molecules inhibitors include natural products, derivatives, and analogs thereof. Small molecule inhibitors can include a covalent cross-linking group capable of forming a covalent cross-link, e.g., with an amino acid side-chain of a target protein.

As used herein, the term “linker” refers to a divalent organic moiety connecting a first moiety (e.g., a macrocyclic moiety) to a second moiety (e.g., a cross-linking group). In some embodiments, the linker results in a compound capable of achieving an IC50 of 2 uM or less in the Ras-RAF disruption assay protocol provided in the Examples below, and provided here:
The purpose of this biochemical assay is to measure the ability of test compounds to facilitate ternary complex formation between a nucleotide-loaded Ras isoform and cyclophilin A; the resulting ternary complex disrupts binding to a BRAF^{RBD} construct, inhibiting Ras signaling through a RAF effector.
In assay buffer containing 25 mM HEPES pH 7.3, 0.002% Tween20, 0.1% BSA, 100 mM NaCl and 5 mM MgCl₂, tagless Cyclophilin A, His6-K-Ras-GMPPNP (or other Ras variant), and GST-BRAF^{RBD} are combined in a 384-well assay plate at final concentrations of 25 μM, 12.5 nM and 50 nM, respectively. Compound is present in plate wells as a 10-point 3-fold dilution series starting at a final concentration of 30 μM. After incubation at 25° C. for 3 hours, a mixture of Anti-His Eu—W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, Em 665/615 nm). Compounds that facilitate disruption of a Ras:RAF complex are identified as those eliciting a decrease in the TR-FRET ratio relative to DMSO control wells.

In some embodiments, the linker comprises 20 or fewer linear atoms. In some embodiments, the linker comprises 15 or fewer linear atoms. In some embodiments, the linker comprises 10 or fewer linear atoms. In some embodiments, the linker has a molecular weight of under 500 g/mol. In some embodiments, the linker has a molecular weight of under 400 g/mol. In some embodiments, the linker has a molecular weight of under 300 g/mol. In some embodiments, the linker has a molecular weight of under 200 g/mol. In some embodiments, the linker has a molecular weight of under 100 g/mol. In some embodiments, the linker has a molecular weight of under 50 g/mol.

As used herein, a “monovalent organic moiety” is less than 500 kDa. In some embodiments, a “monovalent organic moiety” is less than 400 kDa. In some embodiments, a “monovalent organic moiety” is less than 300 kDa. In some embodiments, a “monovalent organic moiety” is less than 200 kDa. In some embodiments, a “monovalent organic moiety” is less than 100 kDa. In some embodiments, a “monovalent organic moiety” is less than 50 kDa. In some embodiments, a “monovalent organic moiety” is less than 25 kDa. In some embodiments, a “monovalent organic moiety” is less than 20 kDa. In some embodiments, a “monovalent organic moiety” is less than 15 kDa. In some embodiments, a “monovalent organic moiety” is less than 10 kDa. In some embodiments, a “monovalent organic moiety” is less than 1 kDa. In some embodiments, a “monovalent organic moiety” is less than 500 g/mol. In some embodiments, a “monovalent organic moiety” ranges between 500 g/mol and 500 kDa.

The term “mutation” as used herein indicates any modification of a nucleic acid or polypeptide which results in an altered nucleic acid or polypeptide. The term “mutation” may include, for example, point mutations, deletions or insertions of single or multiple residues in a polynucleotide, which includes alterations arising within a protein-encoding region of a gene as well as alterations in regions outside of a protein-encoding sequence, such as, but not limited to, regulatory or promoter sequences, as well as amplifications or chromosomal breaks or translocations. In particular embodiments, the mutation results in an amino acid substitution in the encoded-protein.

A “patient” or “subject” is a mammal, e.g., a human, mouse, rat, guinea pig, dog, cat, horse, cow, pig, or non-human primate, such as a monkey, chimpanzee, baboon or rhesus.

The term “prevent” or “preventing” with regard to a subject refers to keeping a disease or disorder from afflicting the subject. Preventing includes prophylactic treatment. For instance, preventing can include administering to the subject a compound disclosed herein before a subject is afflicted with a disease and the administration will keep the subject from being afflicted with the disease.

As used herein, the term “pharmaceutical composition” refers to a compound, such as a compound of the present disclosure, or a pharmaceutically acceptable salt thereof, formulated together with a pharmaceutically acceptable excipient.

A “pharmaceutically acceptable excipient,” as used herein, refers any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The terms “RAS inhibitor” and “inhibitor of [a]RAS” are used interchangeably to refer to any inhibitor that targets, that is, selectively binds to or inhibits a RAS protein.

As used herein, the terms “RAS^{MULTI} inhibitor” or “RAS(MULTI) inhibitor” refer to a RAS inhibitor of at least 3 RAS variants with missense mutations at one of the following positions: 12, 13, 59, 61, or 146. In some embodiments, a RAS^{MULTI} inhibitor refers to a RAS inhibitor of at least 3 RAS variants with missense mutations at one of the following positions: 12, 13, and 61.

As used herein, the term “RAS(ON) inhibitor” refers to an inhibitor that targets, that is, selectively binds to or inhibits, the GTP-bound, active state of RAS (e.g., selective over the GDP-bound, inactive state of RAS). Inhibition of the GTP-bound, active state of RAS includes, for example, the inhibition of oncogenic signaling from the GTP-bound, active state of RAS. In some embodiments, the RAS(ON) inhibitor is an inhibitor that selectively binds to and inhibits the GTP-bound, active state of RAS. In certain embodiments, RAS(ON) inhibitors may also bind to or inhibit the GDP-bound, inactive state of RAS (e.g., with a lower affinity or inhibition constant than for the GTP-bound, active state of RAS). The RAS inhibitors of Formula 0 and Formula I herein, and subformula thereof, are RAS(ON) inhibitors.

The terms “RAS pathway” and “RAS/MAPK pathway” are used interchangeably herein to refer to a signal transduction cascade downstream of various cell surface growth factor receptors in which activation of RAS (and its various isoforms and alleotypes) is a central event that drives a variety of cellular effector events that determine the proliferation, activation, differentiation, mobilization, and other functional properties of the cell. SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTP-accelerating proteins (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various cellular effector functions.

As used herein, the term “resistant to treatment” refers to a treatment of a disorder with a therapeutic agent, where the therapeutic agent is ineffective or where the therapeutic agent was previously effective and has become less effective over time. Resistance to treatment includes acquired resistance to treatment, which refers to a decrease in the efficacy of a treatment over a period of time where the subject is being administered the therapeutic agent. Acquired resistance to treatment may result from the acquisition of a mutation in a target protein that renders the treatment ineffective or less effective. Accordingly, resistance to treatment may persist even after cessation of administration of the therapeutic agent. In particular, a cancer may become resistant to treatment with an immune checkpoint inhibitor following treatment with an immune checkpoint inhibitor. Such cancers are also referred to herein as “immune refractory.” Measurement of a decrease in the efficacy of the treatment will depend on the disorder being treated, and such methods are known to those of skill in the art. For example, efficacy of a cancer treatment may be measured by the progression of the disease. An effective treatment may slow or halt the progression of the disease. A cancer that is resistant to treatment with a therapeutic agent, e.g., an immune checkpoint inhibitor, may fail to slow or halt the progression of the disease.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl,” as used herein, represents an —S(O)₂— group.

A “therapeutic agent” is any substance, e.g., a compound or composition, capable of treating a disease or disorder. In some embodiments, therapeutic agents that are useful in connection with the present disclosure include RAS inhibitors and cancer chemotherapeutics. Many such therapeutic agents are known in the art and are disclosed herein.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

The term “thiocarbonyl,” as used herein, refers to a —C(S)— group.

The term “treatment” (also “treat” or “treating”), in its broadest sense, refers to any administration of a substance (e.g., a compound of the present disclosure) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder or condition or of a subject who exhibits only early signs of the disease, disorder, or condition. Alternatively, or additionally, in some embodiments, treatment may be administered to a subject who exhibits one or more established signs of the relevant disease, disorder or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “vinyl ketone,” as used herein, refers to a group comprising a carbonyl group directly connected to a carbon-carbon double bond.

The term “vinyl sulfone,” as used herein, refers to a group comprising a sulfonyl group directed connected to a carbon-carbon double bond.

The term “wild-type” refers to an entity having a structure or activity as found in nature in a “normal” (as contrasted with mutant, diseased, altered, etc.) state or context. Those of ordinary skill in the art will appreciate that wild-type genes and polypeptides often exist in multiple different forms (e.g., alleles).

The term “ynone,” as used herein, refers to a group comprising the structure

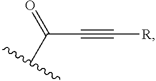

wherein R is any any chemically feasible substituent described herein.

### RAS Inhibitors

Provided herein are compounds that inhibit RAS and uses thereof. Also provided are pharmaceutical compositions including one or more RAS inhibitor compounds, or a pharmaceutically acceptable salt thereof, and a pharmaceutically acceptable excipient. RAS inhibitor compounds may be used in methods of inhibiting RAS (e.g., in a subject or in a cell) and methods of treating cancer, as described herein. In some embodiments, a compound of the present disclosure is or acts as a prodrug, such as with respect to administration to a cell or to a subject in need thereof.

RAS proteins (KRAS, HRAS, and NRAS) play an essential role in various human cancers and are therefore appropriate targets for anticancer therapy. Indeed, mutations in RAS proteins account for approximately 30% of all human cancers in the United States, many of which are fatal. Dysregulation of RAS proteins by activating mutations, overexpression, or upstream activation is common in human tumors, and activating mutations in RAS are frequently found in human cancer. RAS converts between a GDP-bound “off” and a GTP-bound “on” state. The conversion between states is facilitated by interplay between a guanine nucleotide exchange factor (GEF) protein (e.g., SOS1), which loads RAS with GTP, and a GTPase-activating protein (GAP) protein (e.g., NF1), which hydrolyzes GTP, thereby inactivating RAS. Additionally, the SH2 domain-containing protein tyrosine phosphatase-2 (SHP2) associates with the receptor signaling apparatus and becomes active upon RTK activation, and then promote RAS activation. Mutations in RAS proteins can lock the protein in the “on” state resulting in a constitutively active signaling pathway that leads to uncontrolled cell growth. For example, activating mutations at codon 12 in RAS proteins function by inhibiting both GAP-dependent and intrinsic hydrolysis rates of GTP, significantly skewing the population of RAS mutant proteins to the “on” (GTP-bound) state (RAS(ON)), leading to oncogenic MAPK signaling. Notably, RAS exhibits a picomolar affinity for GTP, enabling RAS to be activated even in the presence of low concentrations of this nucleotide. Mutations at codons 13 (e.g., G13D) and 61 (e.g., Q61K) of RAS are also responsible for oncogenic activity in some cancers.

Oncogenic pathways, like KRAS, and the presence of immunosuppressive cell populations, like tumor associated macrophages (TAMs) and myeloid-derived suppressor cells (MDSCs), have emerged as biological mechanisms of T cell exclusion (Liu et al, Theranostics 2021).

KRAS mutations predominate in lung, pancreatic and colon cancers and dictate the immunosuppressive tumor microenvironments (TME) in these tumors (Gu et al., Cancers 2021). Oncogenic KRAS mutations mediate autocrine effects and crosstalk with the TME by inducing several inflammatory cytokines, chemokines and signaling pathways that promote carcinogenesis and resistance to immunotherapies (Hamarsheh et al, Nat. Commun. 2020).

RAS inhibitors described herein may sensitize immune refractory lung cancers to immunotherapy. It is suggested herein that therapies using a RAS inhibitor described herein may alter the tumor immune infiltrate comprising of T-cells, B-cells, APCs, monocytes, MDSCs, TAMs, neutrophils, other monocyte-derived cells, tumor-associated stroma, cancer stem cells, or mesenchymal stem cells and result in an enhanced anti-tumor therapeutic effect. In some embodiments, a RAS inhibitor described herein may sensitize a subject to immunotherapy, such as checkpoint inhibitor therapy.

RAS inhibitors of the present disclosure may form a high affinity three-component complex, or conjugate, between a synthetic ligand and two intracellular proteins which do not interact under normal physiological conditions: the target protein of interest (e.g., RAS), and a widely expressed cytosolic chaperone (presenter protein) in the cell (e.g., cyclophilin A). More specifically, in some embodiments, the inhibitors of RAS described herein induce a new binding pocket in RAS by driving formation of a high affinity tri-complex, or conjugate, between the RAS protein and the widely expressed cytosolic chaperone, cyclophilin A (CYPA). Without being bound by theory, the inventors believe that one way the inhibitory effect on RAS is affected by compounds of the invention and the complexes, or conjugates, they form is by steric occlusion of the interaction site between RAS and downstream effector molecules, such as RAF, which are required for propagating the oncogenic signal. See, e.g., WO 2021/091982, incorporated herein by reference in its entirety.

In some embodiments of the combination therapies described herein, the second RAS inhibitor is a RAS(MULTI) inhibitor (e.g., a compound of Formula AI or a subformula thereof, or a compound of Table A1 or Table A2). RAS(MULTI) inhibitors of Formula AI are described in WO 2021/091956 which is incorporated herein by reference in its entirety. In some embodiments, the RAS inhibitor is a compound, or a pharmaceutically acceptable salt thereof, having the structure of Formula A00:

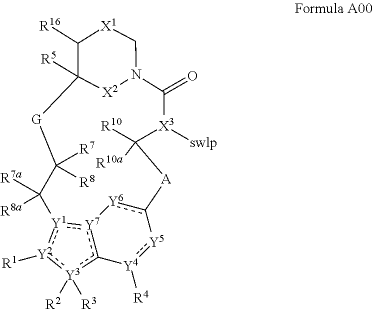

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
swIp (Switch I/P-loop) refers to an organic moiety that non-covalently binds to both the Switch I binding pocket and residues 12 or 13 of the P-loop of a Ras protein (see, e.g., Johnson et al., 292:12981-12993 (2017), incorporated herein by reference);
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′} C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R¹⁰ is hydrogen, halo, hydroxy, C₁-C₃ alkoxy, or C₁-C₃ alkyl;
R^{10a} is hydrogen or halo; and
R¹⁶ is hydrogen or C₁-C₃ alkyl (e.g., methyl). In some embodiments, the resulting compound is capable of achieving an IC50 of 2 uM or less (e.g., 1.5 uM, 1 uM, 500 nM, or 100 nM or less) in the Ras-RAF disruption assay protocol described herein.

In some embodiments, the RAS inhibitor is a compound, or pharmaceutically acceptable salt thereof, of structural Formula AI:

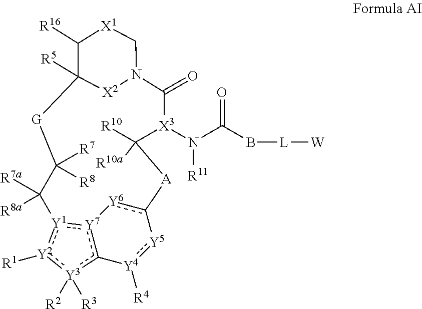

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is hydrogen, cyano, S(O)₂R′, optionally substituted amino, optionally substituted amido, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′} C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R¹⁶ is hydrogen or C₁-C₃ alkyl (e.g., methyl).

In some embodiments, the RAS inhibitor is a compound, or pharmaceutically acceptable salt thereof, of structural Formula AIa:

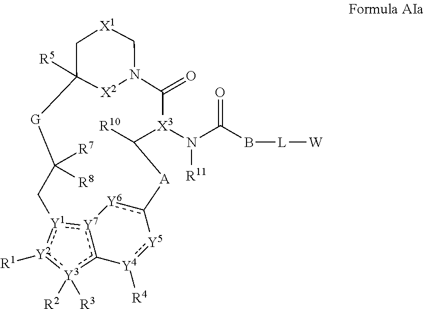

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′} C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments, the RAS inhibitor is a compound, or pharmaceutically acceptable salt thereof, of structural Formula AIb:

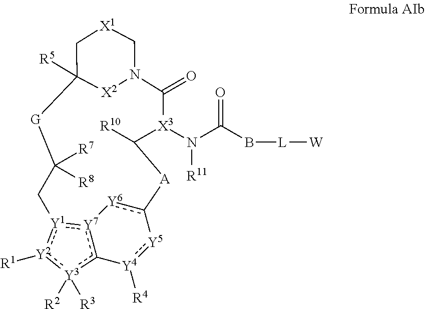

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of Formula AI and subformula thereof, G is optionally substituted C₁-C₄ heteroalkylene.

In some embodiments, the RAS inhibitor is a compound having the structure of Formula AIc, or a pharmaceutically acceptable salt thereof:

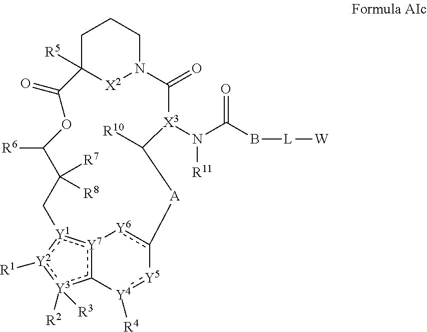

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′} C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of Formula AI and subformula thereof, X² is NH. In some embodiments of Formula AI and subformula thereof, X³ is CH.

In some embodiments of Formula AI and subformula thereof, R¹¹ is hydrogen. In some embodiments of Formula AI and subformula thereof, R¹¹ is C₁-C₃ alkyl. In some embodiments of Formula AI and subformula thereof, R¹¹ is methyl.

In some embodiments, the RAS inhibitor is a compound having the structure of Formula AId, or a pharmaceutically acceptable salt thereof:

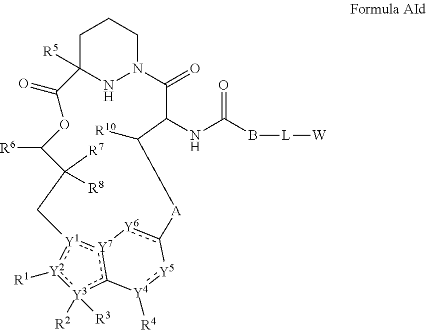

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of Formula AI and subformula thereof, X¹ is optionally substituted C₁-C₂ alkylene. In some embodiments, X¹ is methylene. In some embodiments, X¹ is methylene substituted with a C₁-C₆ alkyl group or a halogen. In some embodiments, X¹ is —CH(Br)—. In some embodiments, X¹ is —CH(CH₃)—.

In some embodiments of Formula AI and subformula thereof, R³ is absent.

In some embodiments of Formula AI and subformula thereof, R⁴ is hydrogen.

In some embodiments of Formula AI and subformula thereof, R⁵ is hydrogen. In some embodiments of Formula AI and subformula thereof, R⁵ is C₁-C₄ alkyl optionally substituted with halogen. In some embodiments of Formula AI and subformula thereof, R⁵ is methyl.

In some embodiments of of Formula AI and subformula thereof, Y⁴ is C. In some embodiments of Formula AI and subformula thereof, Y⁵ is CH. In some embodiments of Formula AI and subformula thereof, Y⁶ is CH. In some embodiments of Formula AI and subformula thereof, Y¹ is C. In some embodiments of Formula AI and subformula thereof, Y² is C. In some embodiments of Formula AI and subformula thereof, Y³ is N. In some embodiments of Formula AI and subformula thereof, Y⁷ is C.

In some embodiments, the RAS inhibitor is a compound having the structure of Formula Ale, or a pharmaceutically acceptable salt thereof:

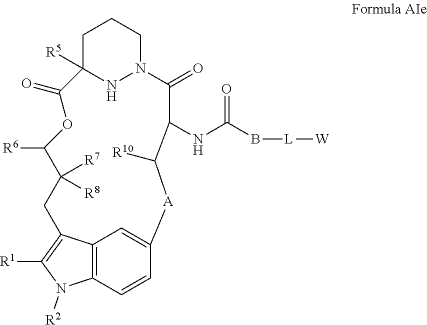

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′} C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of Formula AI and subformula thereof, R⁶ is hydrogen.

In some embodiments of Formula AI and subformula thereof, R² is hydrogen, cyano, optionally substituted C₁-C₆ alkyl, optionally substituted 3 to 6-membered cycloalkyl, or optionally substituted 3 to 6-membered heterocycloalkyl. In some embodiments of Formula AI and subformula thereof, R² is optionally substituted C₁-C₆ alkyl, such as ethyl. In some embodiments of Formula AI and subformula thereof, R² is fluoro C₁-C₆ alkyl, such as —CH₂CH₂F, —CH₂CHF₂, or —CH₂CF₃.

In some embodiments of Formula AI and subformula thereof, R⁷ is optionally substituted C₁-C₃ alkyl. In some embodiments of Formula AI and subformula thereof, R⁷ is C₁-C₃ alkyl.

In some embodiments of Formula AI and subformula thereof, R⁸ is optionally substituted C₁-C₃ alkyl. In some embodiments of Formula AI and subformula thereof, R⁸ is C₁-C₃ alkyl, such as methyl.

In some embodiments, the RAS inhibitor is a compound having the structure of Formula Alf, or a pharmaceutically acceptable salt thereof:

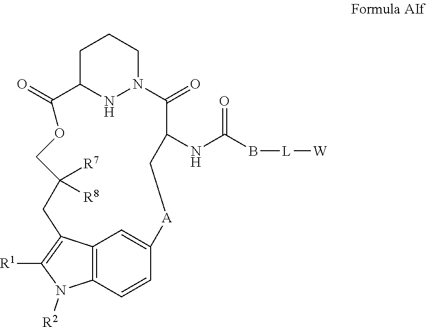

wherein A optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula AI and subformula thereof, R¹ is 5 to 10-membered heteroaryl. In some embodiments, R¹ is optionally substituted 6-membered aryl or optionally substituted 6-membered heteroaryl.

In some embodiments of Formula AI and subformula thereof, R₁ is

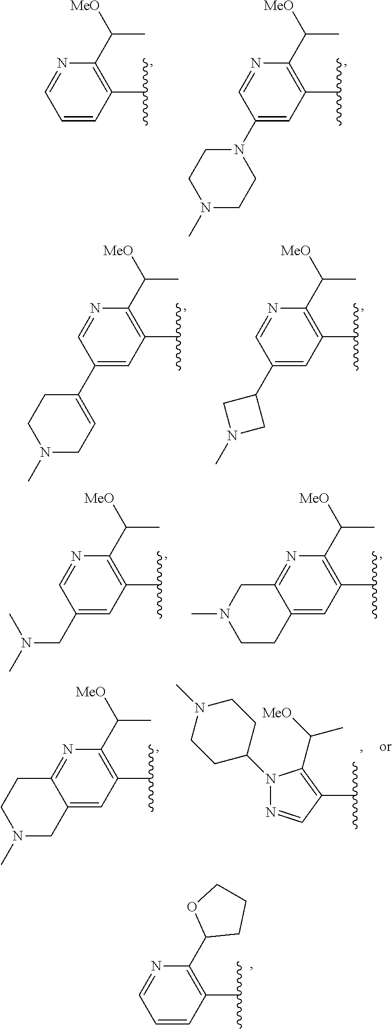

or a stereoisomer thereof. In some embodiments, R₁ is

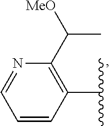 [SMILES: *c1cccnc1C(C)OC]

or a stereoisomer thereof. In some embodiments, R₁ is

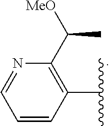 [SMILES: *c1cccnc1[C@H](C)OC]

In some embodiments, R₁ is

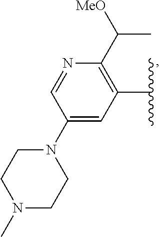 [SMILES: *c1cc(N2CCN(C)CC2)cnc1C(C)OC]

or a stereoisomer thereof. In some embodiments, R₁ is

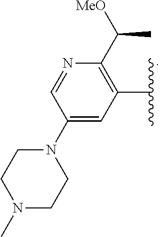 [SMILES: *c1cc(N2CCN(C)CC2)cnc1[C@H](C)OC]

In some embodiments, the RAS inhibitor is a compound having the structure of Formula AIg, or a pharmaceutically acceptable salt thereof:

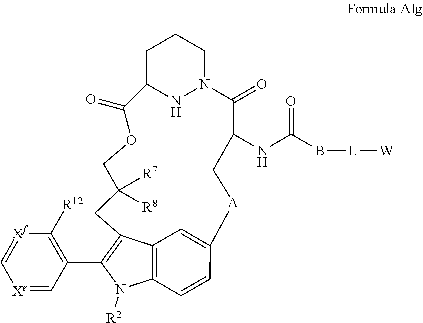

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R² is C₁-C₆ alkyl or 3 to 6-membered cycloalkyl;
R⁷ is C₁-C₃ alkyl;
R⁸ is C₁-C₃ alkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} is N, CH, or CR¹⁷;
X^{f} is N or CH;
R¹² is optionally substituted C₁-C₆ alkyl or optionally substituted C₁-C₆ heteroalkyl; and
R¹⁷ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments of Formula AI and subformula thereof, X^{e} is N and X^{f} is CH. In some embodiments, X^{e} is CH and X^{f} is N. In some embodiments, X^{e} is CR¹⁷ and X^{f} is N.

In some embodiments of Formula AI and subformula thereof, R¹² is optionally substituted C₁-C₆ heteroalkyl. In some embodiments, R¹² is

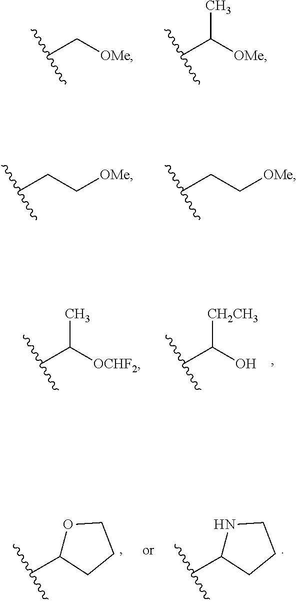

In some embodiments, the RAS inhibitor is a compound having the structure of Formula AIh, or a pharmaceutically acceptable salt thereof:

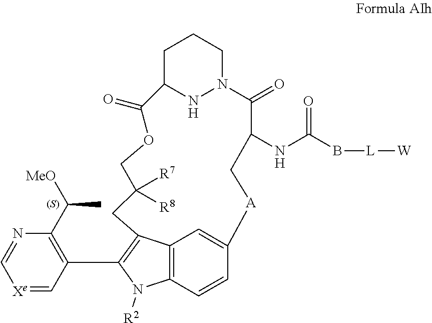

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R² is C₁-C₆ alkyl or 3 to 6-membered cycloalkyl;
R⁷ is C₁-C₃ alkyl;
R⁸ is C₁-C₃ alkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} is CH, or CR¹⁷; and
R¹⁷ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments, the RAS inhibitor is a compound having the structure of Formula Ali, or a pharmaceutically acceptable salt thereof:

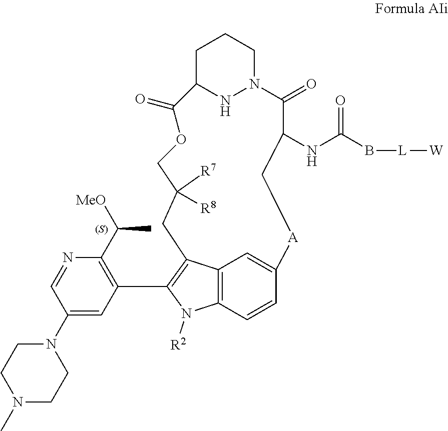

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula AI and subformula thereof, A is optionally substituted 6-membered arylene. In some embodiments, A has the structure:

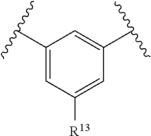

wherein R¹³ is hydrogen, hydroxy, amino, cyano, optionally substituted C₁-C₆ alkyl, or optionally substituted C₁-C₆ heteroalkyl. In some embodiments, R¹³ is hydrogen. In some embodiments, R¹³ is hydroxy. In some embodiments, A is an optionally substituted 5 to 10-membered heteroarylene. In some embodiments, A is:

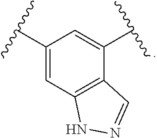 [SMILES: *c1cc(*)c2cn[nH]c2c1]

In some embodiments, A is optionally substituted 5 to 6-membered heteroarylene. In some embodiments, A is:

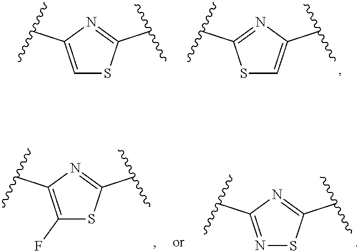 [SMILES: *c1csc(*)n1 ; *c1csc(*)n1 ; *c1nc(*)c(F)s1 ; *c1nsc(*)n1]

In some embodiments, A is

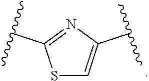 [SMILES: *c1csc(*)n1]

In some embodiments of Formula AI and subformula thereof, B is —CHR⁹—. In some embodiments, R⁹ is optionally substituted C₁-C₆ alkyl or optionally substituted 3 to 6-membered cycloalkyl. In some embodiments, R⁹ is:

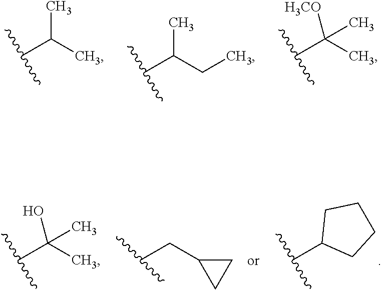

In some embodiments, R⁹ is:

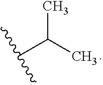 [SMILES: *C(C)C]

In some embodiments, R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula AI and subformula thereof, B is optionally substituted 6-membered arylene.

In some embodiments, B is 6-membered arylene. In some embodiments, B is:

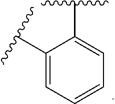 [SMILES: *c1ccccc1*]

In some embodiments B is absent.

In some embodiments of Formula AI and subformula thereof, R⁷ is methyl.

In some embodiments of Formula AI and subformula thereof, R⁸ is methyl.

In some embodiments of Formula AI and subformula thereof, R¹⁶ is hydrogen.

In some embodiments of Formula AI and subformula thereof, the linker is the structure of Formula AII:

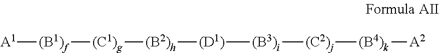

where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₁-C₃ cycloalkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to —(B³)ᵢ—(C²)ⱼ—(B⁴)ₖ-A². In some embodiments, the linker is acyclic. In some embodiments, the linker has the structure of Formula AIIa:

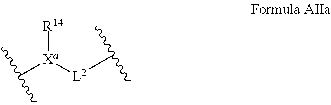

wherein X^{a} is absent or N;
R¹⁴ is absent, hydrogen or optionally substituted C₁-C₆ alkyl or optionally substituted C₁-C₃ cycloalkyl; and
L² is absent, —C(O)—, —SO₂—, optionally substituted C₁-C₄ alkylene or optionally substituted C₁-C₄ heteroalkylene, wherein at least one of X^{a}, R¹⁴, or L² is present. In some embodiments, the linker has the structure:

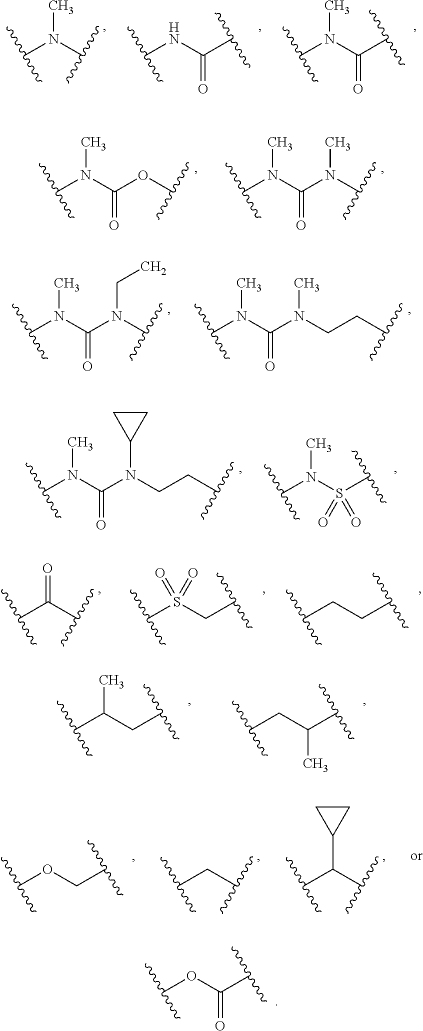

In some embodiments, L is

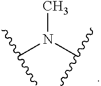 [SMILES: *N(*)C]

In some embodiments, L is

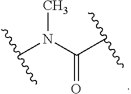 [SMILES: *C(=O)N(*)C]

In some embodiments, linker is or comprises a cyclic group. In some embodiments of Formula AI and subformula thereof, the linker has the structure of Formula AIIb:

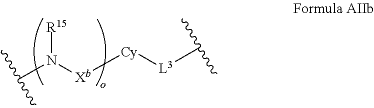

wherein o is 0 or 1;
X^{b} is C(O) or SO₂;
R¹⁵ is hydrogen or optionally substituted C₁-C₆ alkyl;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —C(O)—, —SO₂—, optionally substituted C₁-C₄ alkylene or optionally substituted C₁-C₄ heteroalkylene. In some embodiments, the linker has the structure:

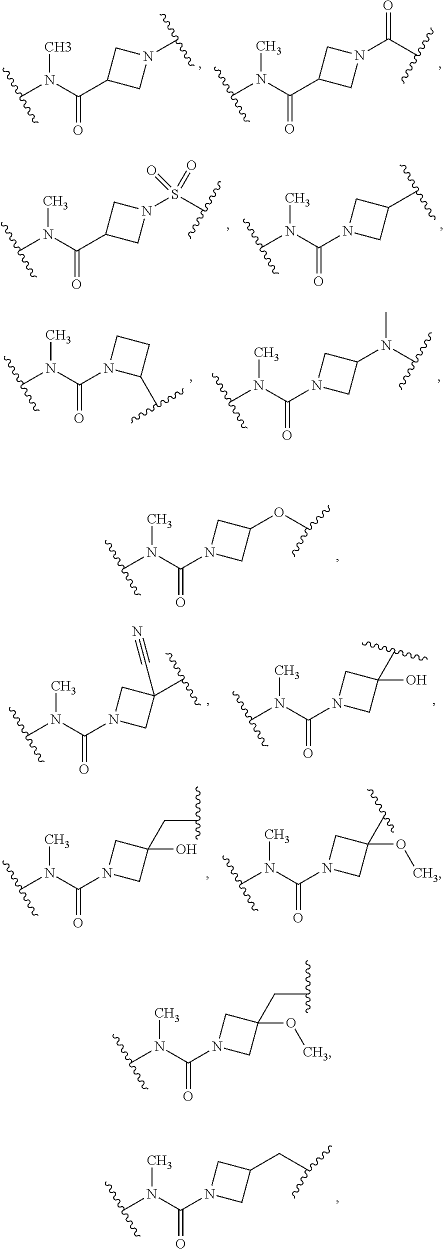

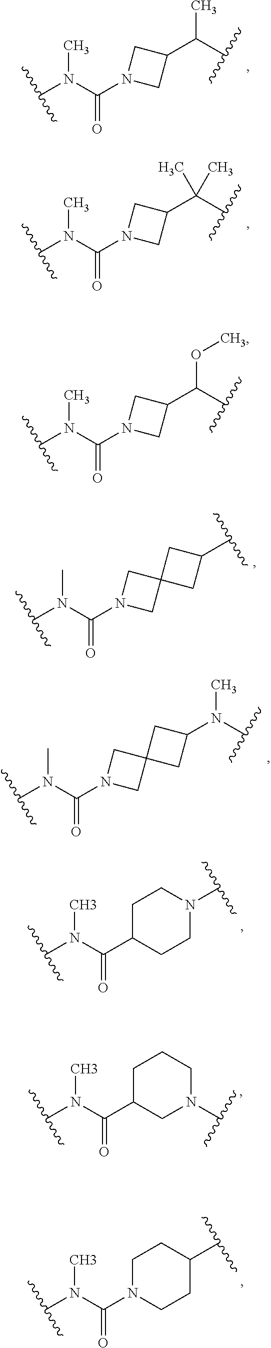

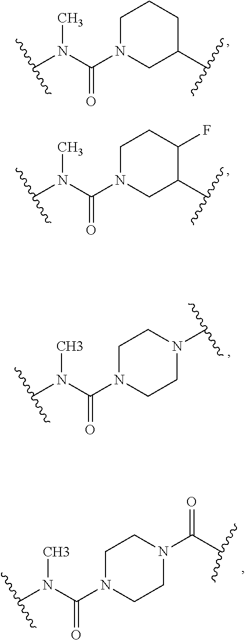 [SMILES: *C1CCCN(C(=O)N(*)C)C1 ; *C1CN(C(=O)N(*)C)CCC1F ; *N1CCN(C(=O)N(*)C)CC1 ; *C(=O)N1CCN(C(=O)N(*)C)CC1]

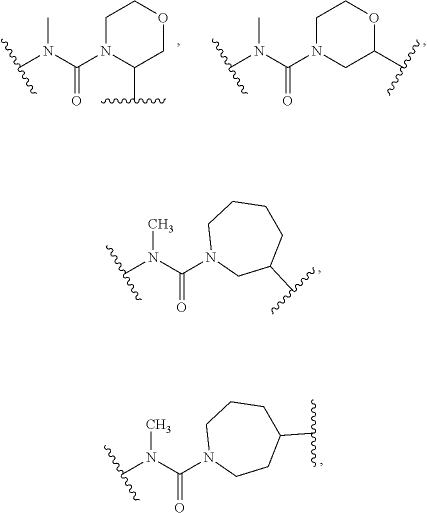 [SMILES: *C1COCCN1C(=O)N(*)C ; *C1CN(C(=O)N(*)C)CCO1 ; *C1CCCCN(C(=O)N(*)C)C1 ; *C1CCCN(C(=O)N(*)C)CC1]

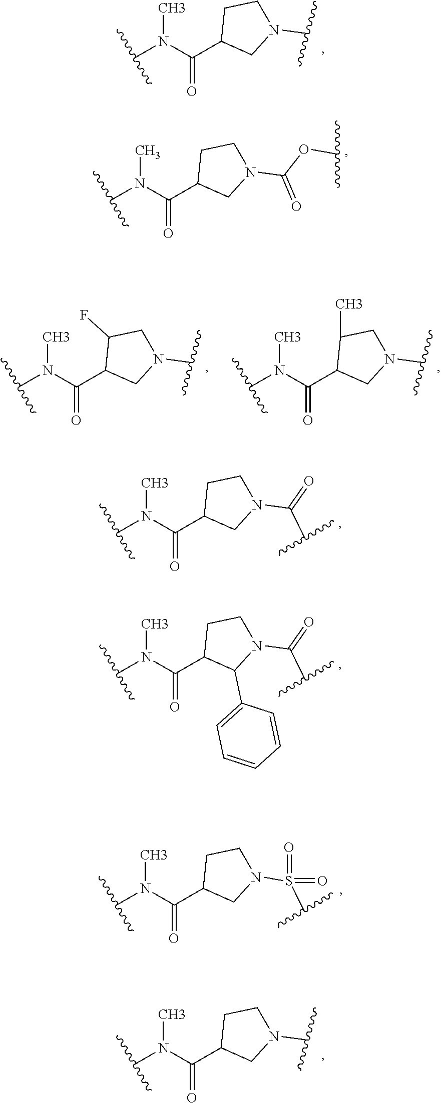

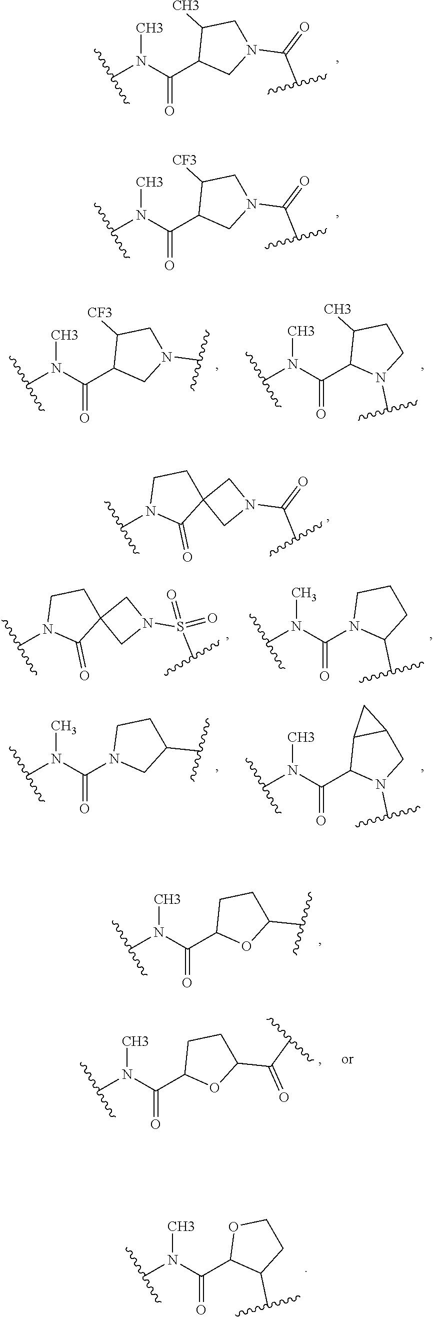

In some embodiments of Formula AI and subformula thereof, W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 8-membered heterocycloalkyl, optionally substituted 3 to 8-membered cycloalkyl, or 3 to 8-membered heteroaryl.

In some embodiments of Formula AI and subformula thereof, W is hydrogen. In some embodiments of Formula AI and subformula thereof, W is optionally substituted amino. In some embodiments of Formula AI and subformula thereof, W is —NHCH₃ or —N(CH₃)₂. In some embodiments of Formula AI and subformula thereof, W is optionally substituted C₁-C₄ alkoxy. In some embodiments, W is methoxy or iso-propoxy. In some embodiments of Formula AI and subformula thereof, W is optionally substituted C₁-C₄ alkyl. In some embodiments, W is methyl, ethyl, iso-propyl, tert-butyl, or benzyl. In some embodiments of Formula AI and subformula thereof, W is optionally substituted amido. In some embodiments, W is

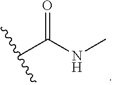 [SMILES: *C(=O)NC]

In some embodiments, W is

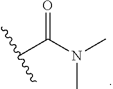 [SMILES: *C(=O)N(C)C]

In some embodiments of Formula AI and subformula thereof, W is optionally substituted C₁-C₄ hydroxyalkyl. In some embodiments, W is

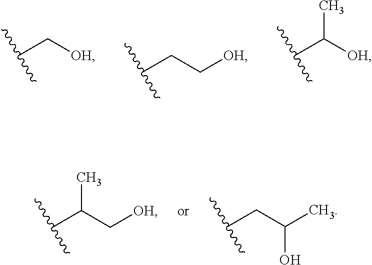

In some embodiments of Formula AI and subformula thereof, W is optionally substituted C₁-C₄ aminoalkyl. In some embodiments, W is

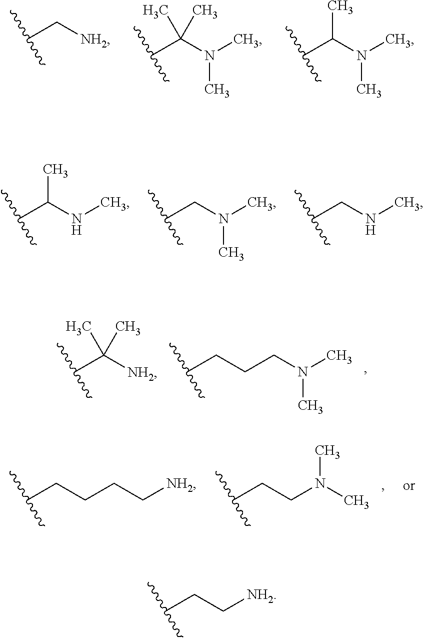

In some embodiments of Formula AI and subformula thereof, W is optionally substituted C₁-C₄ haloalkyl. In some embodiments, W is

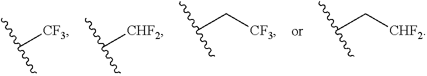

In some embodiments of Formula AI and subformula thereof, W is optionally substituted C₁-C₄ guanidinoalkyl. In some embodiments, W is

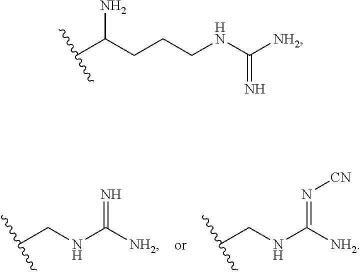 [SMILES: *C(N)CCCNC(=N)N ; *CNC(=N)N ; *CN/C(N)=N/C#N]

In some embodiments of Formula AI and subformula thereof, W is C₀-C₄ alkyl optionally substituted 3 to 11-membered heterocycloalkyl. In some embodiments, W is

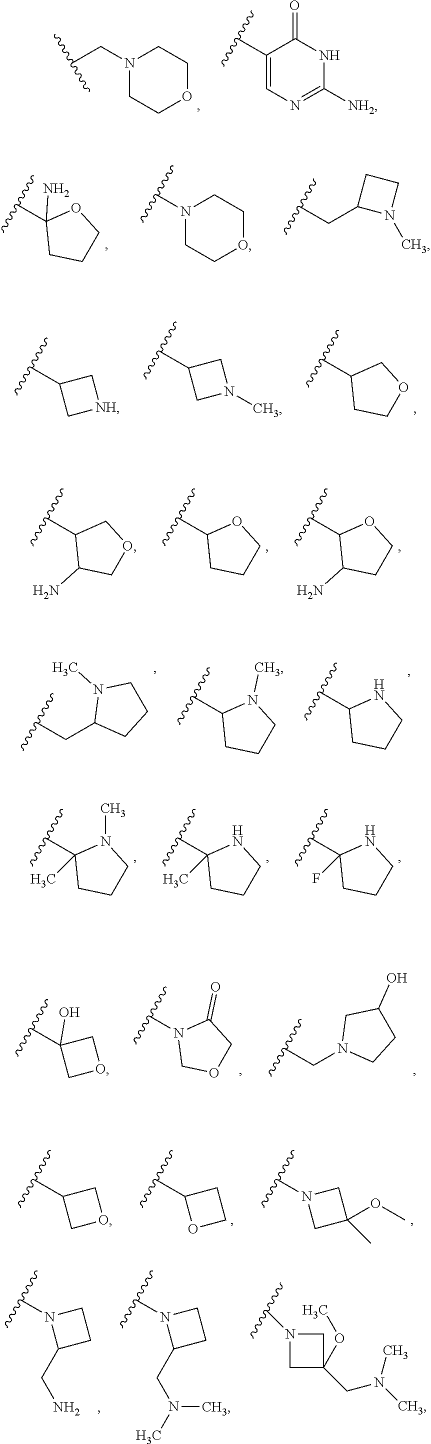

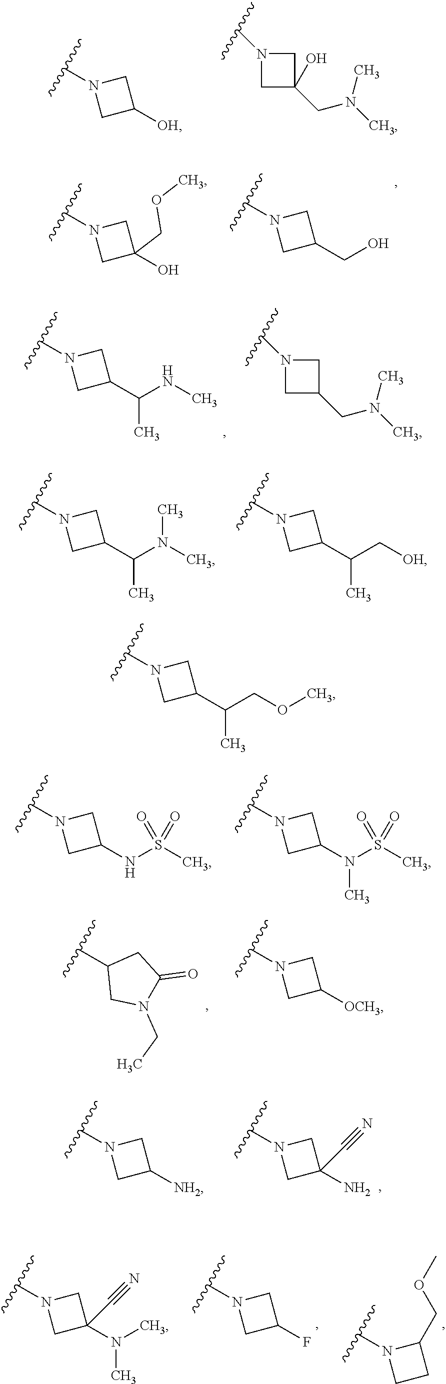

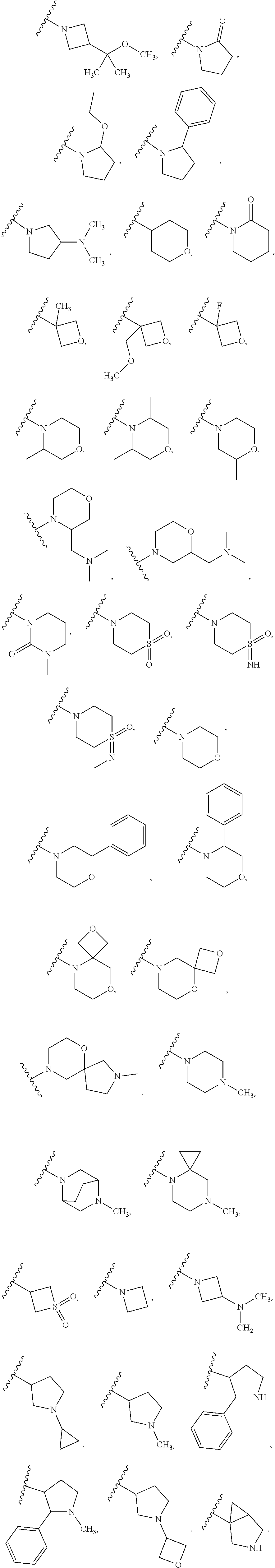

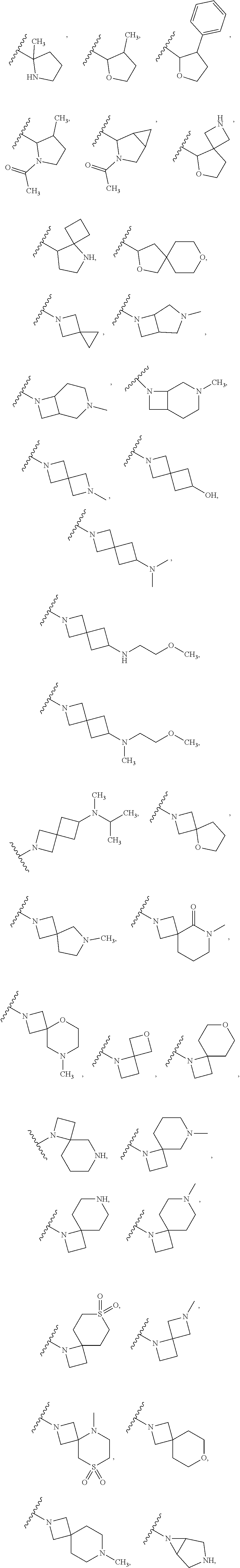

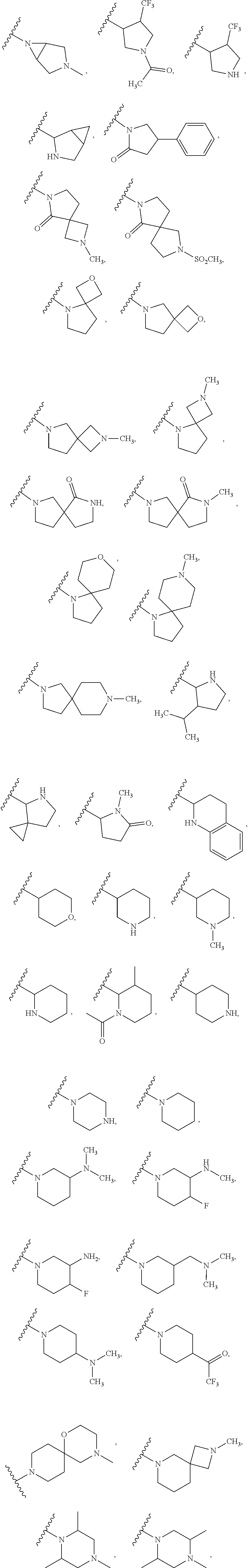

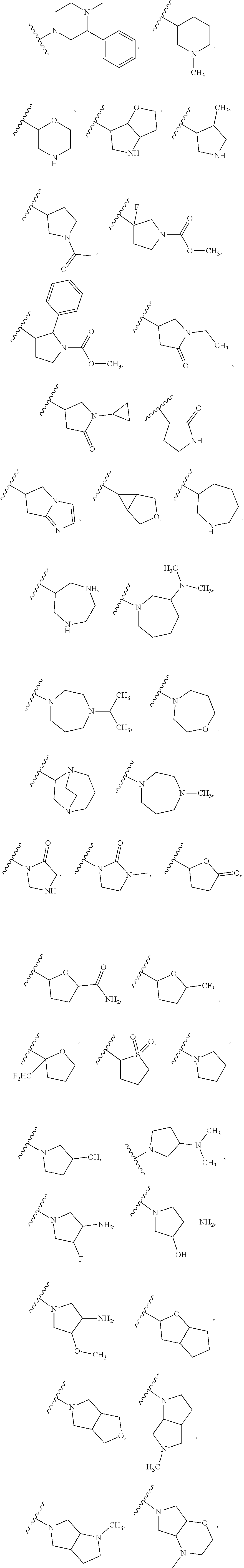

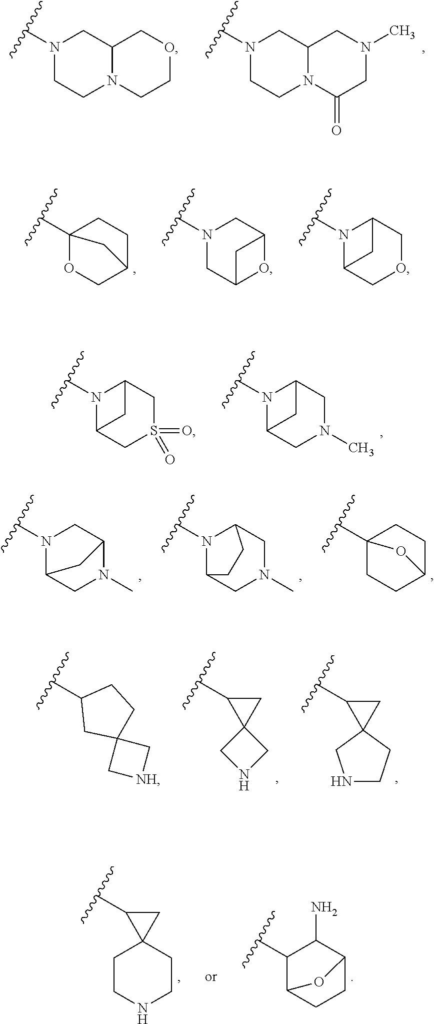

In some embodiments of Formula AI and subformula thereof, W is optionally substituted 3 to 8-membered cycloalkyl. In some embodiments, W is

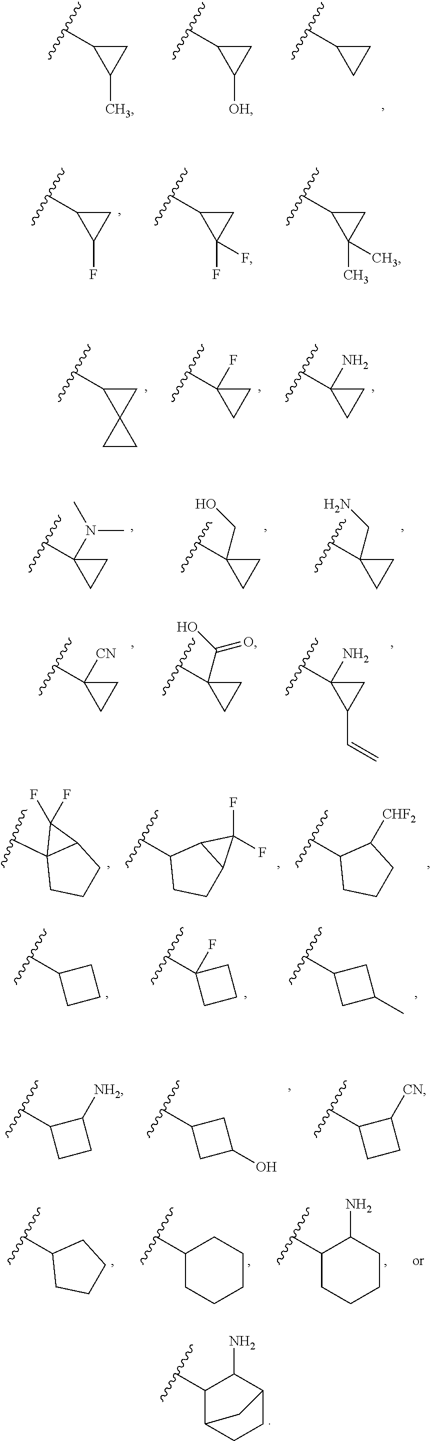

In some embodiments of Formula AI and subformula thereof, W is optionally substituted 3 to 8-membered heteroaryl. In some embodiments, W is

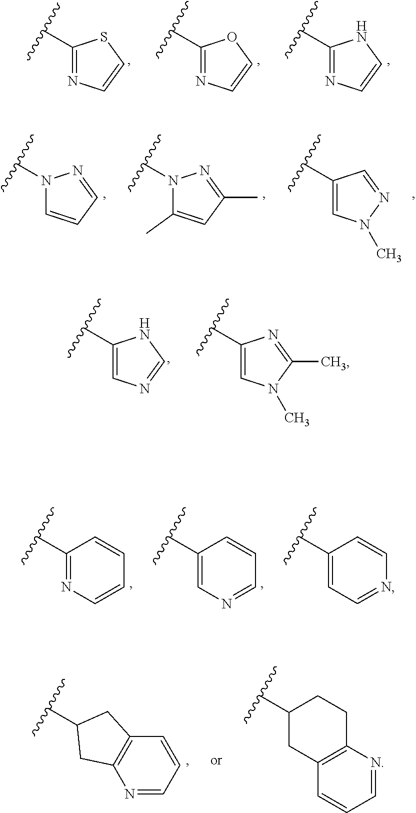

In some embodiments of Formula AI and subformula thereof, W is optionally substituted 6- to 10-membered aryl (e.g., phenyl, 4-hydroxy-phenyl, or 2,4-methoxy-phenyl).

In some embodiments, the RAS inhibitor is a RAS(MULTI) inhibitor. In some embodiments, the RAS(MULTI) inhibitor is selected from Table A1, or a pharmaceutically acceptable salt or stereoisomer thereof. In some embodiments, the RAS(MULTI) inhibitor is selected from Table A1, or a pharmaceutically acceptable salt or atropisomer thereof.

TABLE A1
Certain Compounds of the Present Invention

| Ex# | Structure |
|---|---|
| AA1 | 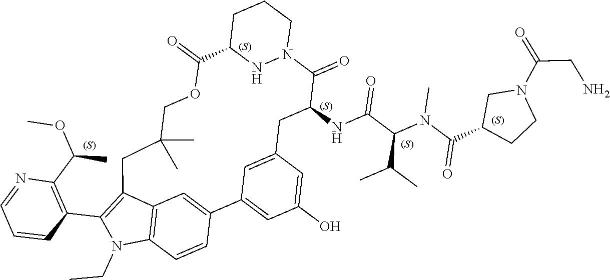 |
| AA2 | 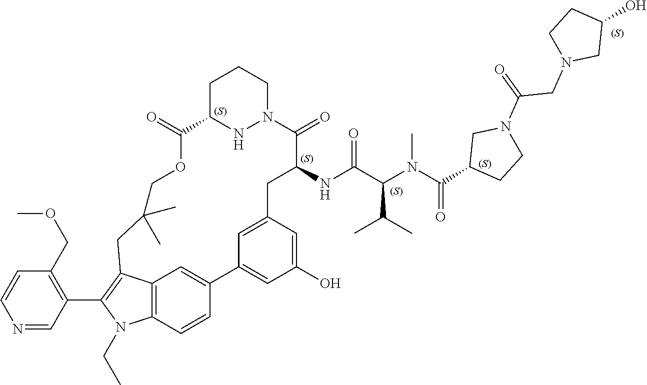 |
| AA3 | 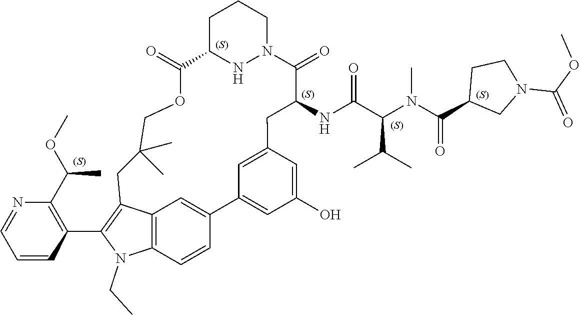 |
| AA4 | 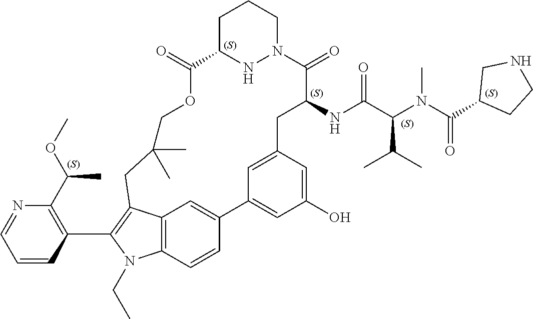 |
| AA5 | 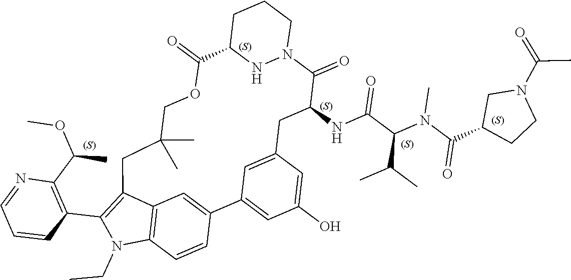 |
| AA6 | 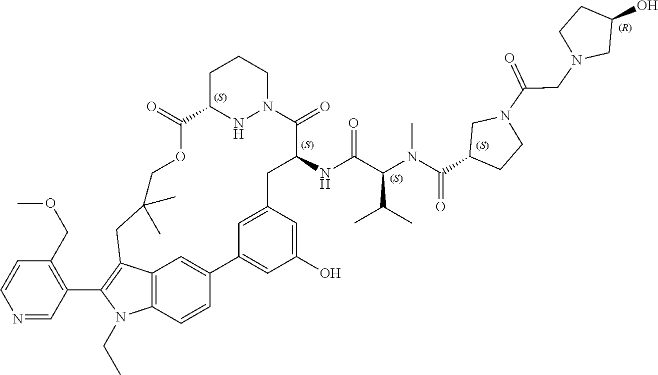 |
| AA7 | 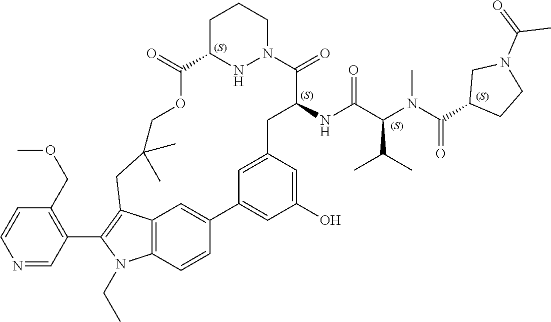 |
| AA8 | 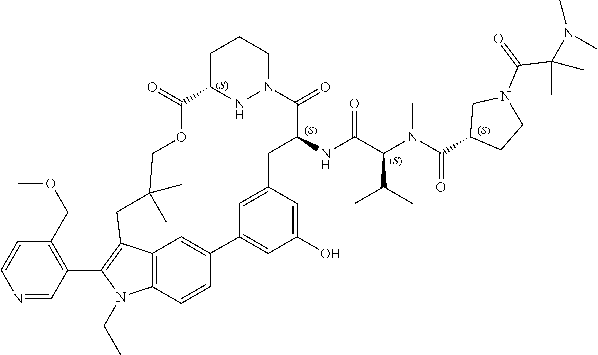 |
| AA9 | 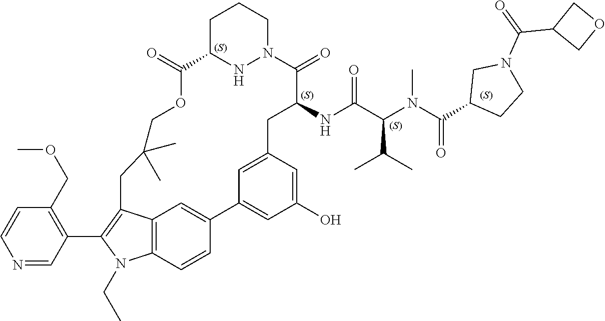 |
| AA10 | 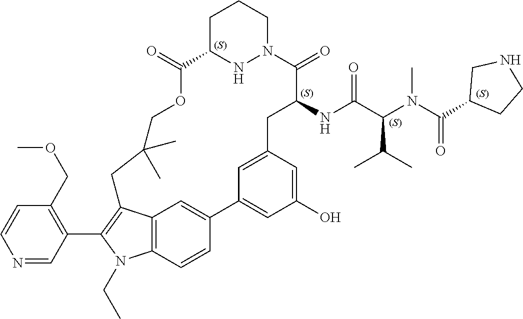 |
| AA11 | 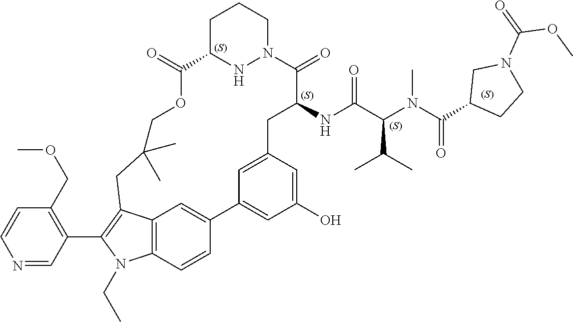 |
| AA12 | 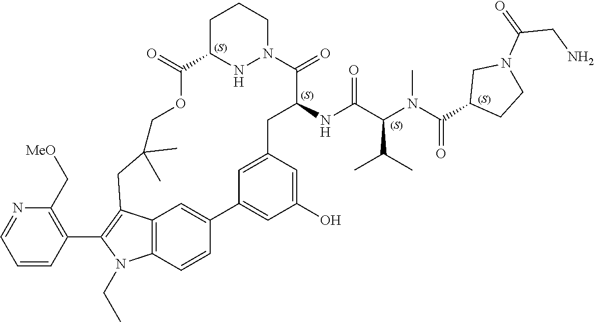 |
| AA13 | 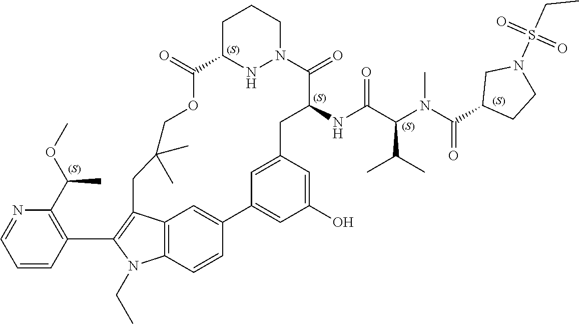 |
| AA14 | 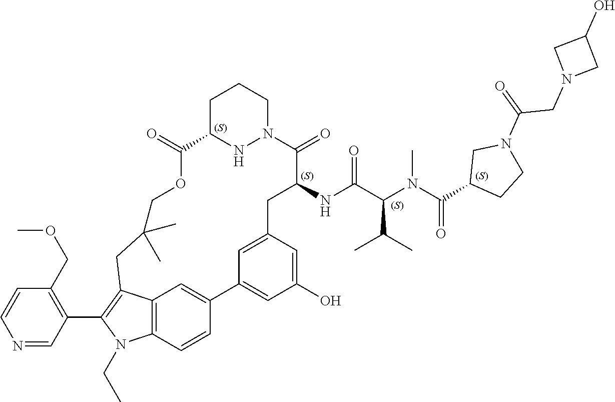 |
| AA15 | 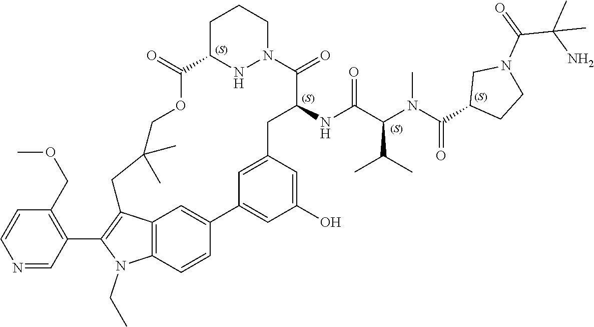 |
| AA16 | 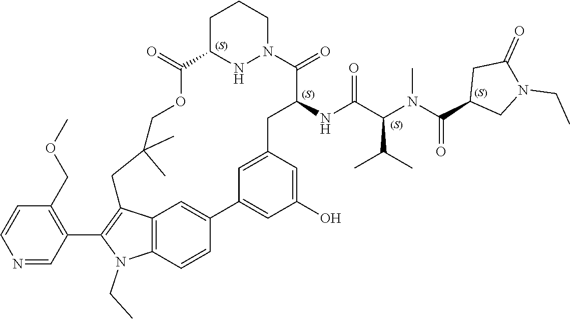 |
| AA17 | 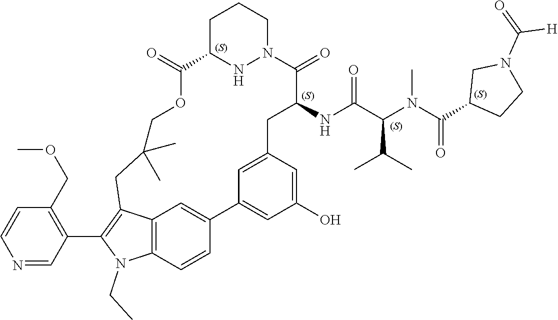 |
| AA18 | 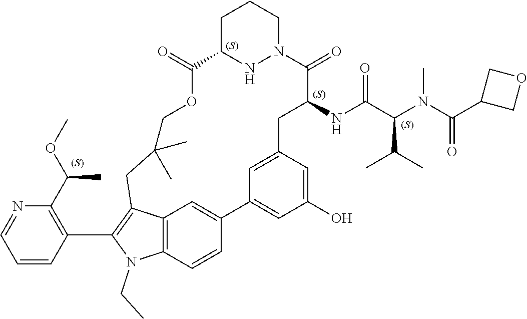 |
| AA19 | 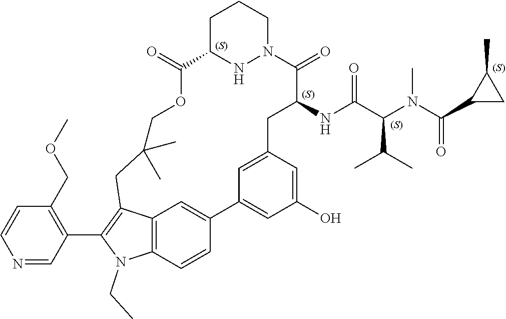 |
| AA20 | 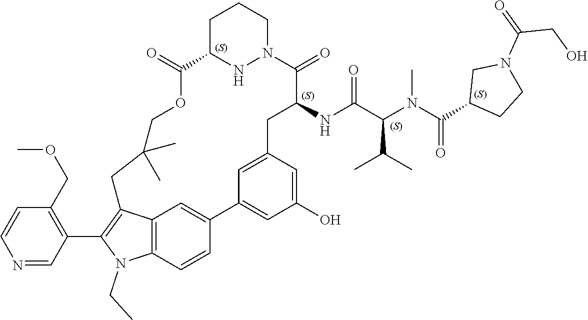 |
| AA21 | 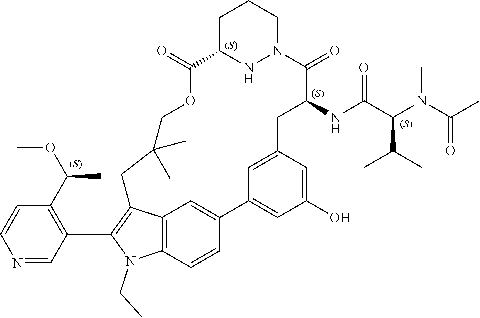 |
| AA22 | 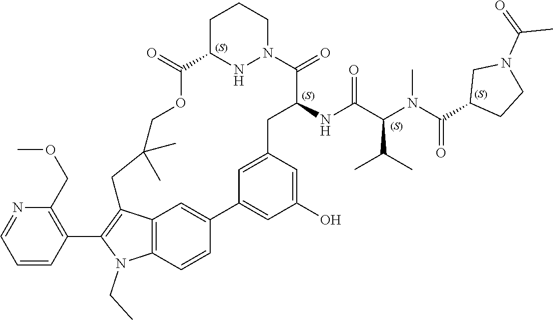 |
| AA23 | 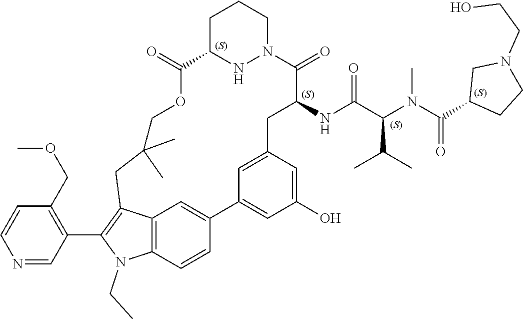 |
| AA24 | 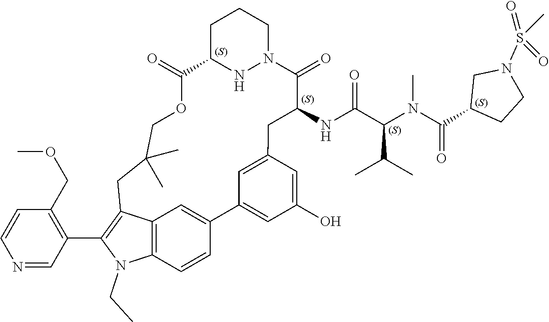 |
| AA25 | 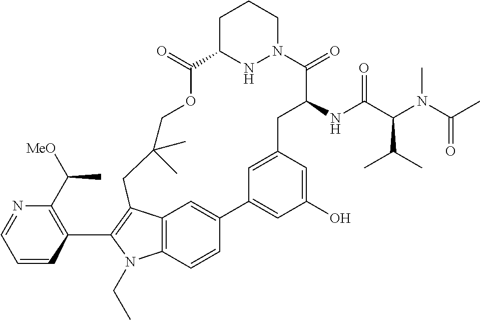 |
| AA26 | 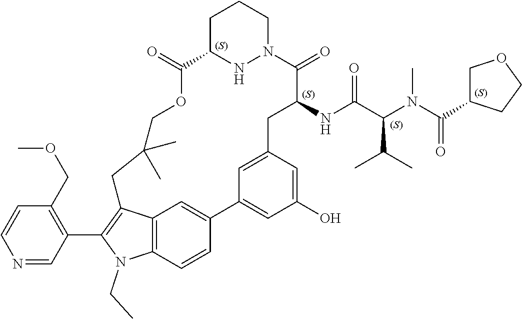 |
| AA27 | 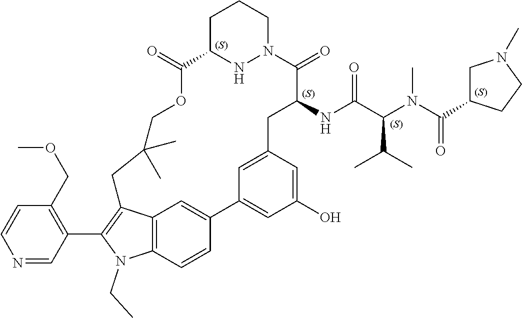 |
| AA28 | 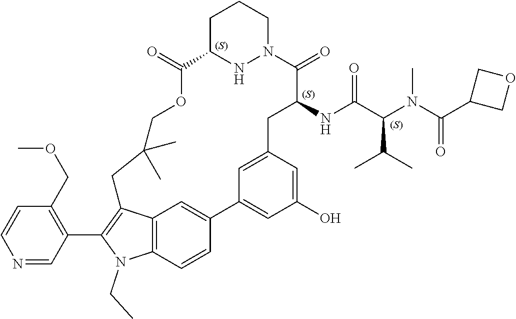 |
| AA29 | 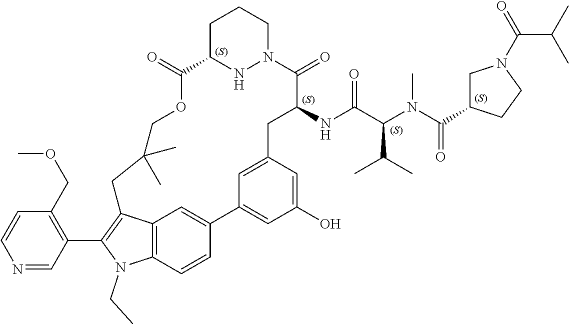 |
| AA30 | 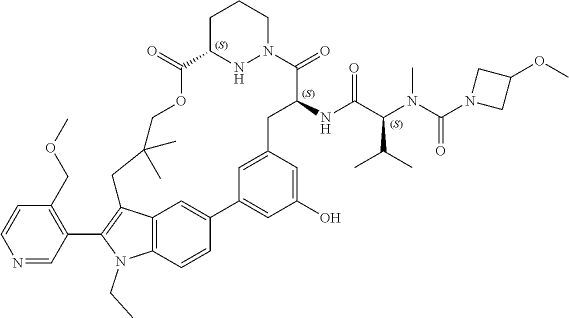 |
| AA31 | 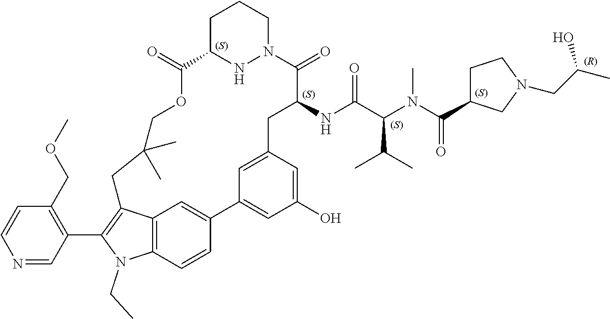 |
| AA32 | 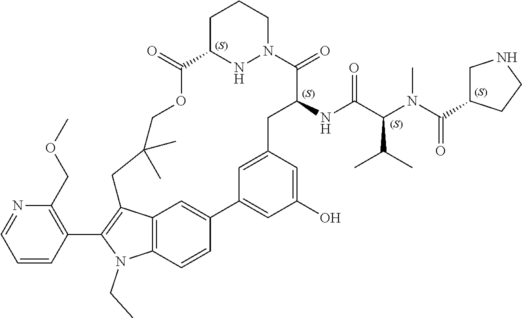 |
| AA33 | 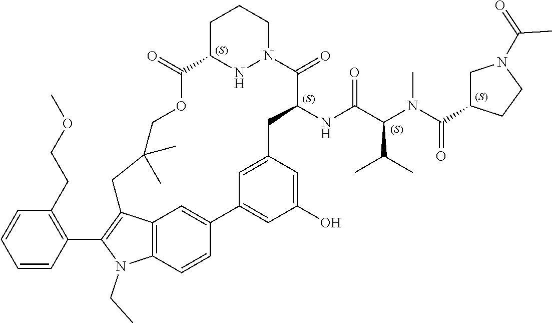 |
| AA34 | 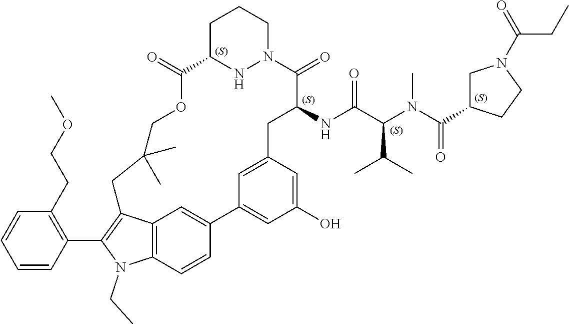 |
| AA35 | 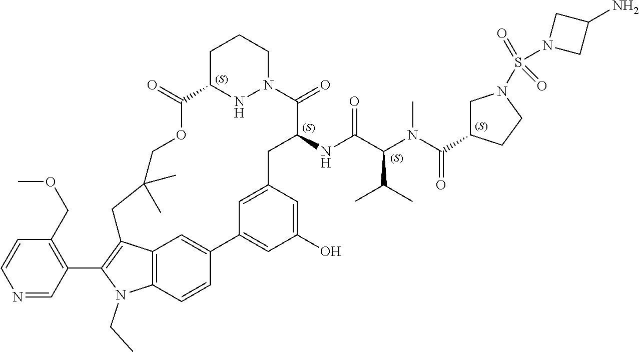 |
| AA36 | 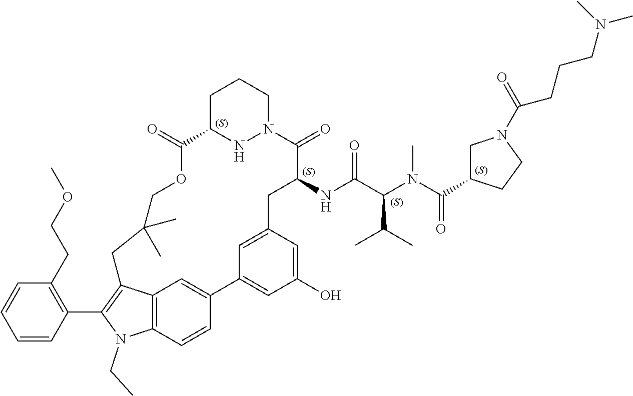 |
| AA37 | 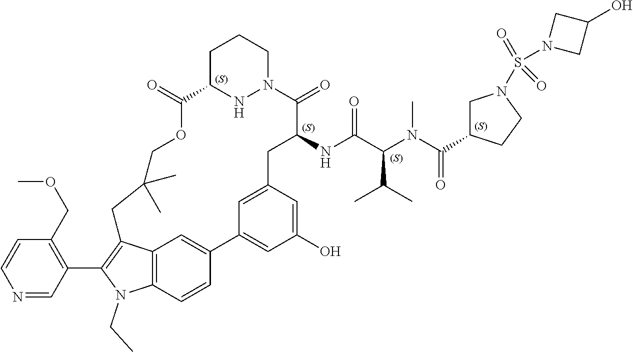 |
| AA38 | 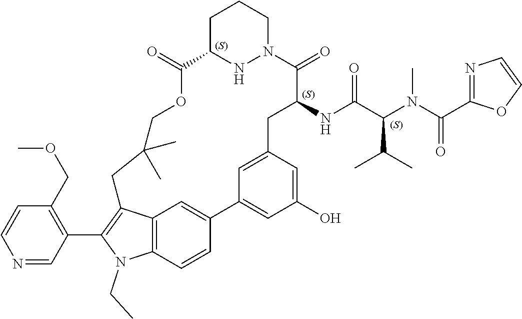 |
| AA39 | 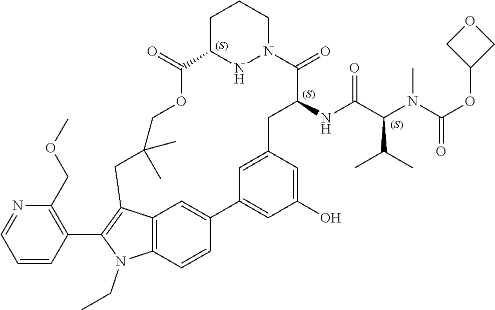 |
| AA40 | 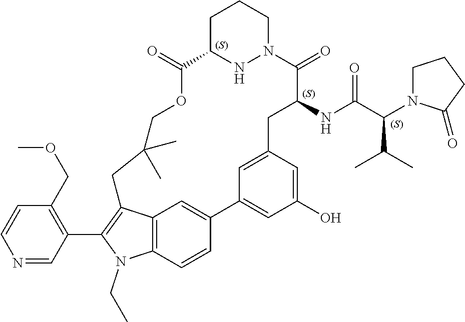 |
| AA41 | 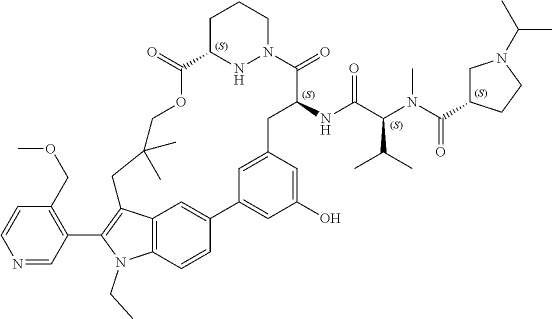 |
| AA42 | 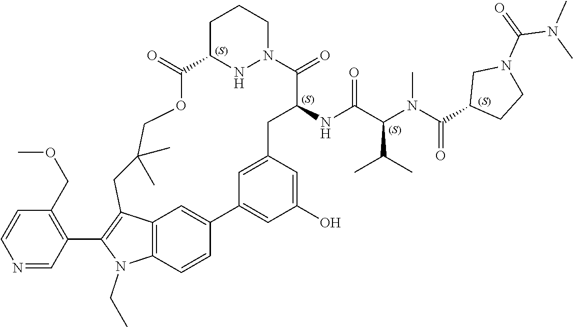 |
| AA43 | 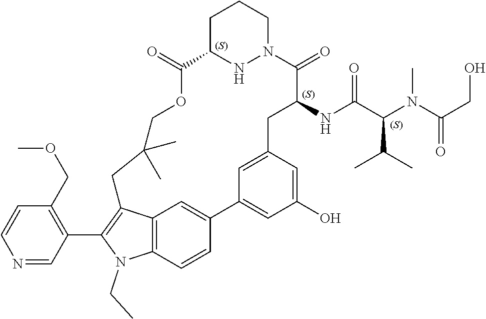 |
| AA44 | 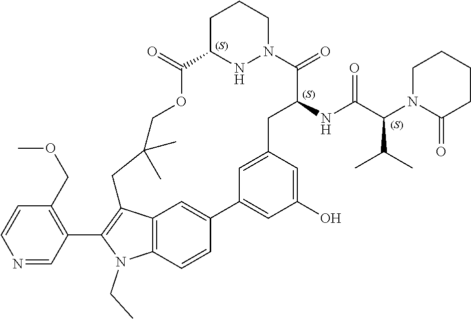 |
| AA45 | 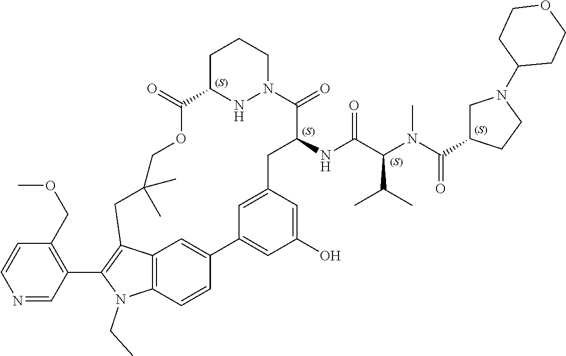 |
| AA46 | 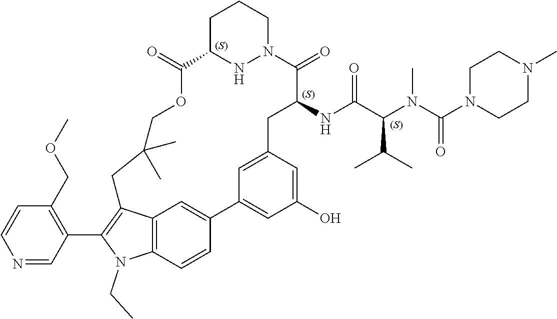 |
| AA47 | 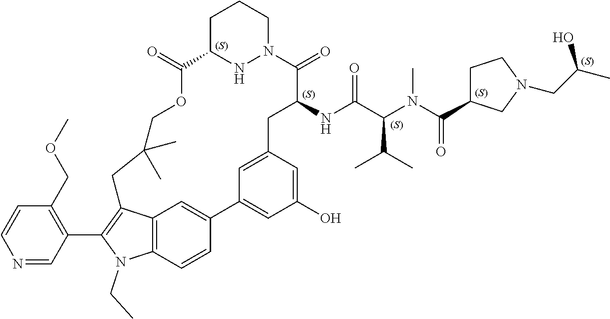 |
| AA48 | 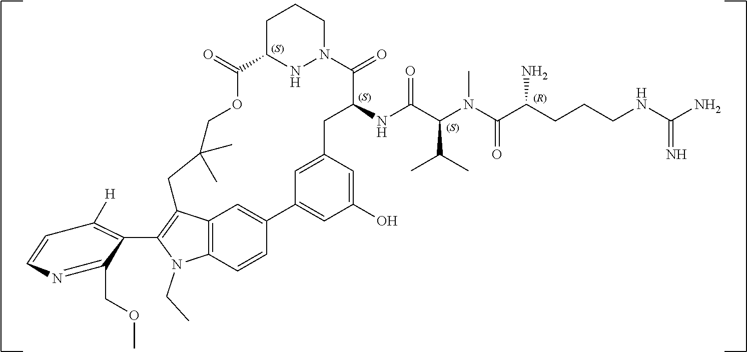 |
| AA49 | 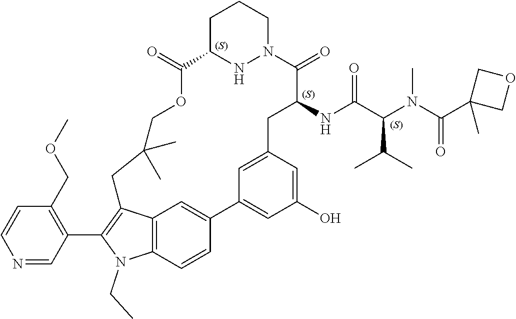 |
| AA50 | 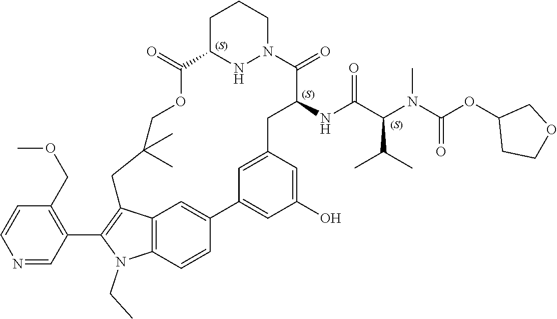 |
| AA51 | 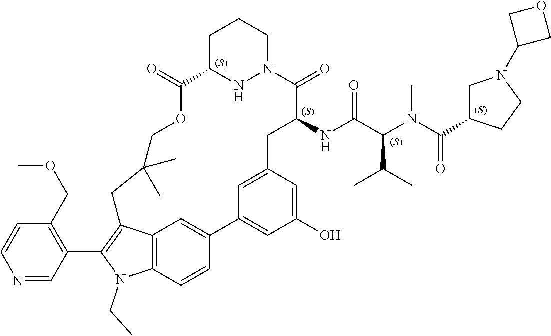 |
| AA52 | 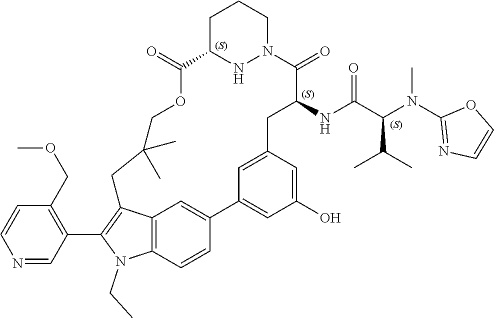 |
| AA53 | 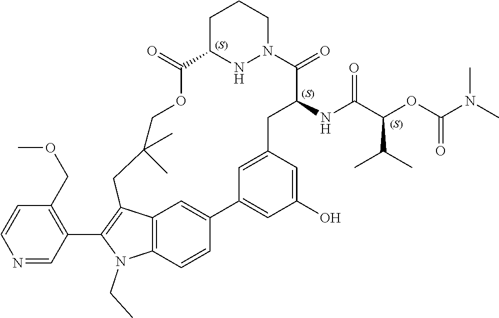 |
| AA54 | 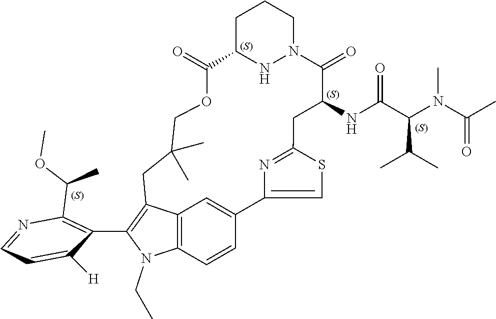 |
| AA55 | 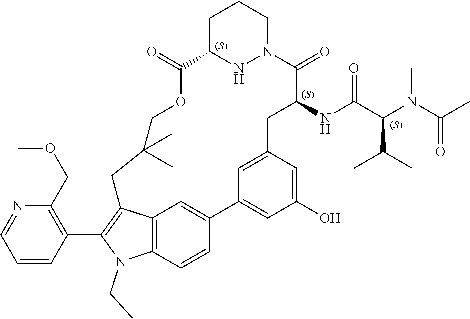 |
| AA56 | 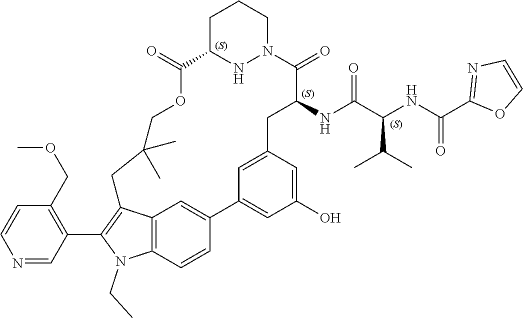 |
| AA57 | 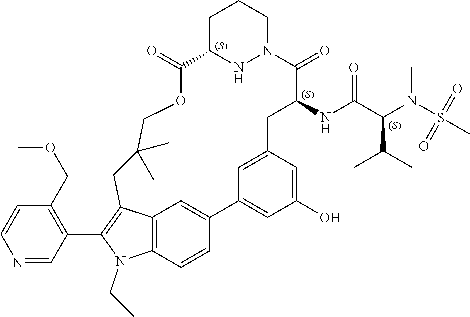 |
| AA58 | 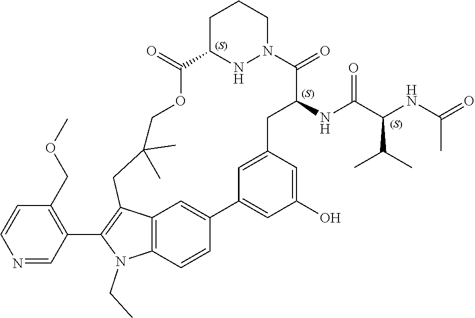 |
| AA59 | 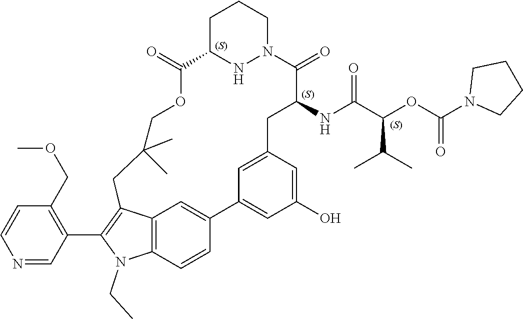 |
| AA60 | 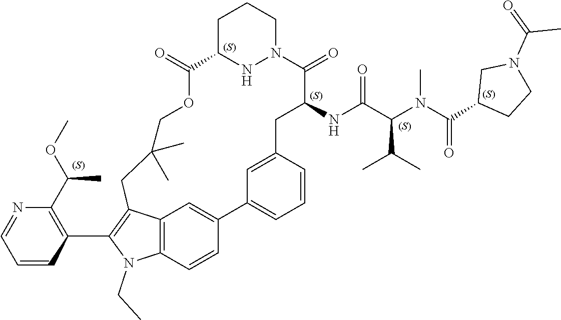 |
| AA61 | 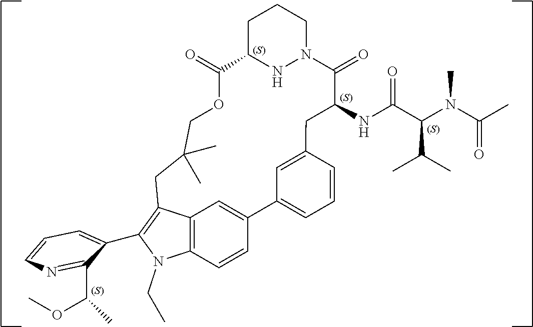 |
| AA62 | 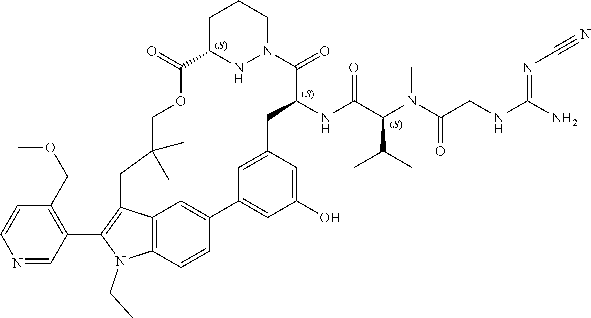 |
| AA63 | 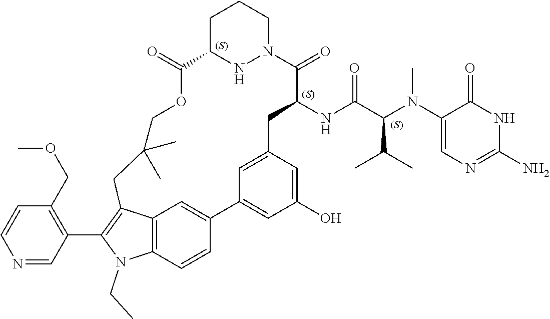 |
| AA64 | 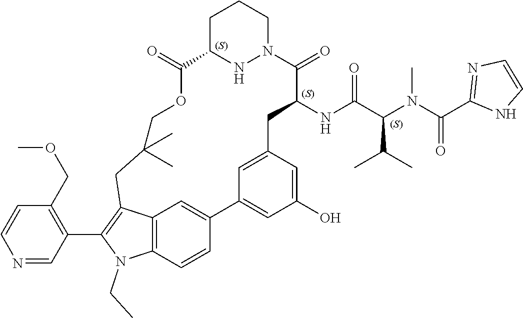 |
| AA65 | 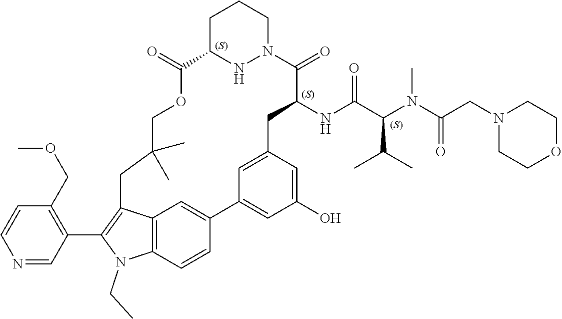 |
| AA66 | 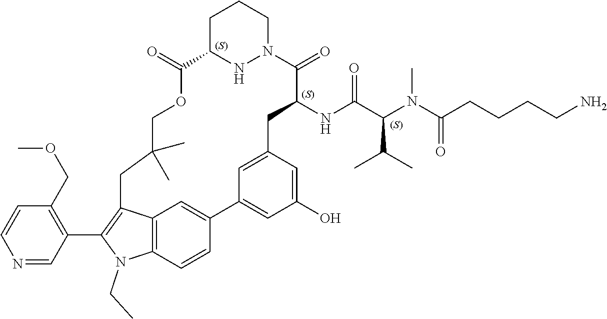 |
| AA67 | 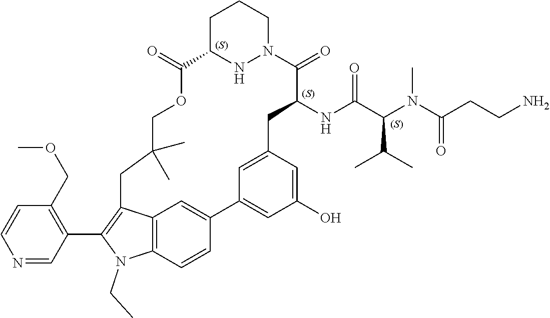 |
| AA68 | 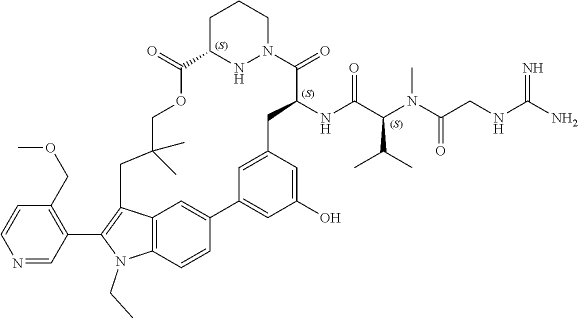 |
| AA69 | 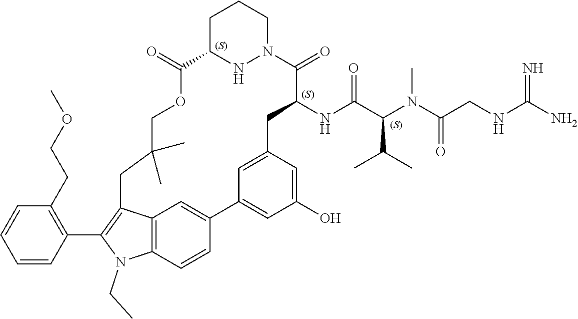 |
| AA70 | 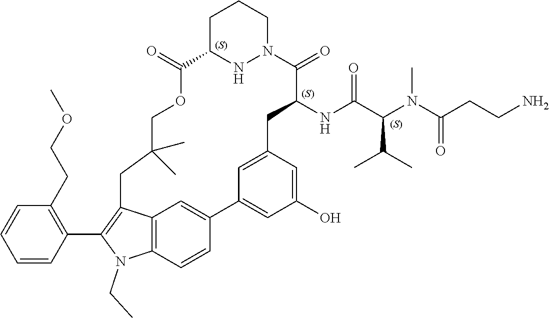 |
| AA71 | 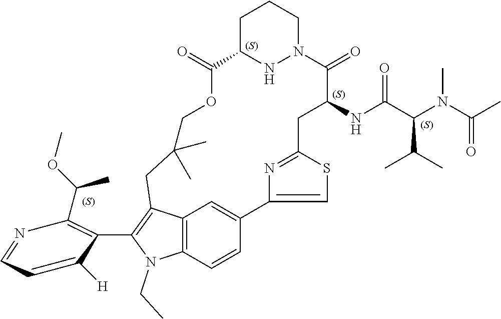 |
| AA72 | 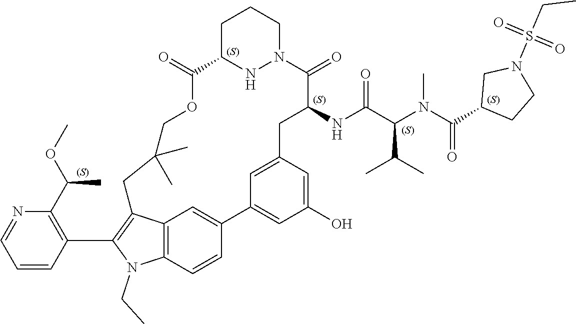 |
| AA73 | 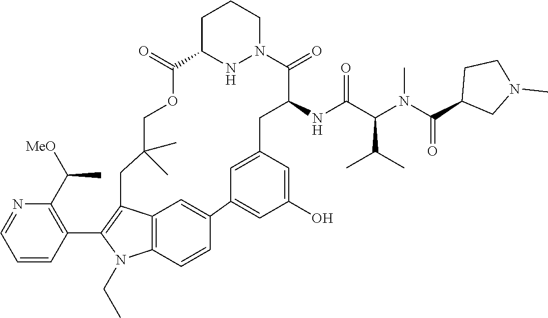 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C)C1)C(=O)N1CCC[C@H](N1)C(=O)OCC(C)(C)C2] |
| AA74 | 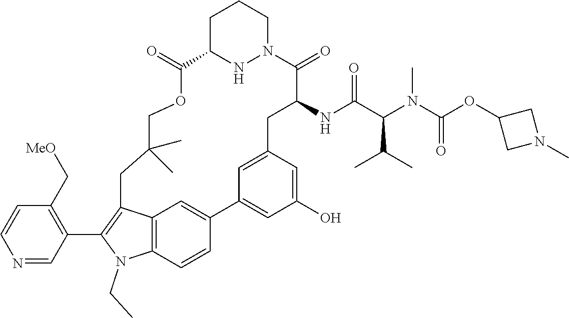 [SMILES: CCn1c(-c2cnccc2COC)c2c3cc(ccc31)-c1cc(O)cc(c1)C[C@H](NC(=O)[C@H](C(C)C)N(C)C(=O)OC1CN(C)C1)C(=O)N1CCC[C@H](N1)C(=O)OCC(C)(C)C2] |
| AA75 | 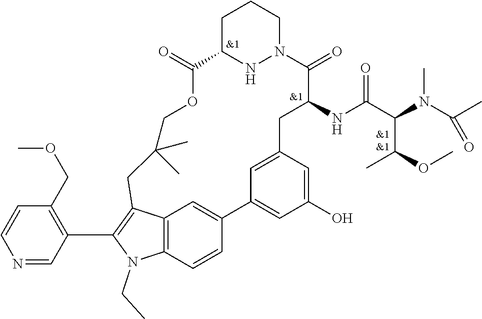 [SMILES: CCn1c(-c2cnccc2COC)c2c3cc(ccc31)-c1cc(O)cc(c1)C[C@H](NC(=O)[C@H]([C@H](C)OC)N(C)C(C)=O)C(=O)N1CCC[C@H](N1)C(=O)OCC(C)(C)C2] |
| AA76 | 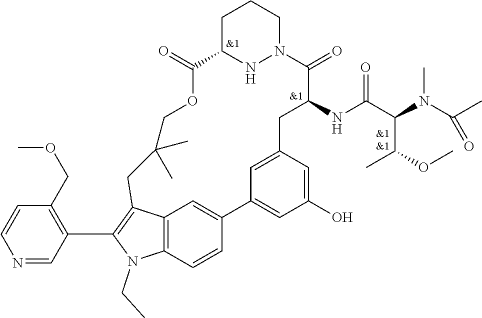 [SMILES: CCn1c(-c2cnccc2COC)c2c3cc(ccc31)-c1cc(O)cc(c1)C[C@H](NC(=O)[C@H]([C@@H](C)OC)N(C)C(C)=O)C(=O)N1CCC[C@H](N1)C(=O)OCC(C)(C)C2] |
| AA77 | 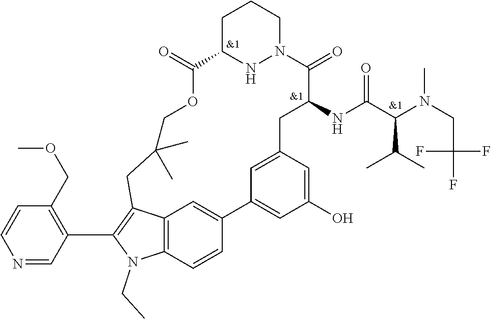 [SMILES: CCn1c(-c2cnccc2COC)c2c3cc(ccc31)-c1cc(O)cc(c1)C[C@H](NC(=O)[C@H](C(C)C)N(C)CC(F)(F)F)C(=O)N1CCC[C@H](N1)C(=O)OCC(C)(C)C2] |
| AA78 | 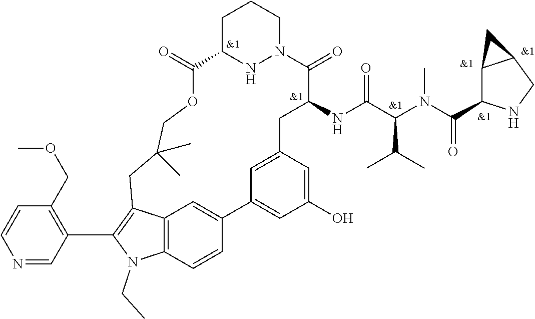 [SMILES: CCn1c(-c2cnccc2COC)c2c3cc(ccc31)-c1cc(O)cc(c1)C[C@H](NC(=O)[C@H](C(C)C)N(C)C(=O)[C@@H]1NC[C@@H]3C[C@@H]31)C(=O)N1CCC[C@H](N1)C(=O)OCC(C)(C)C2] |
| AA79 | 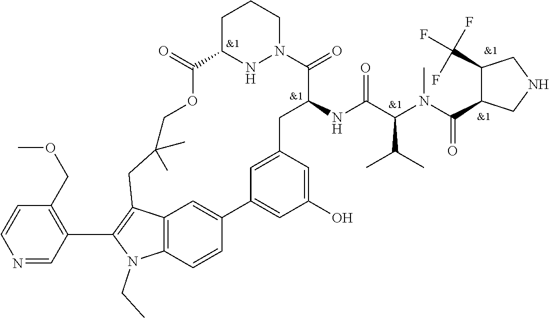 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CNC[C@@H]1C(F)(F)F)C(=O)N1CCC[C@H](N1)C(=O)OCC(C)(C)C2] |
| AA80 | 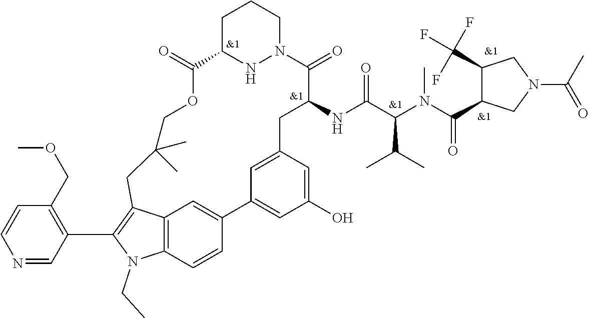 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CN(C(C)=O)C[C@@H]1C(F)(F)F)C(=O)N1CCC[C@H](N1)C(=O)OCC(C)(C)C2] |
| AA81 | 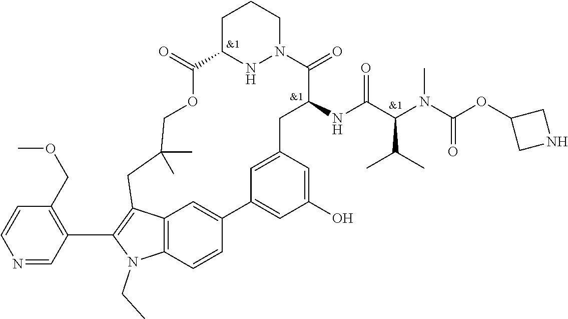 [SMILES: CCn1c(-c2cnccc2COC)c2c3cc(ccc31)-c1cc(O)cc(c1)C[C@H](NC(=O)[C@H](C(C)C)N(C)C(=O)OC1CNC1)C(=O)N1CCC[C@H](N1)C(=O)OCC(C)(C)C2] |
| AA82 | 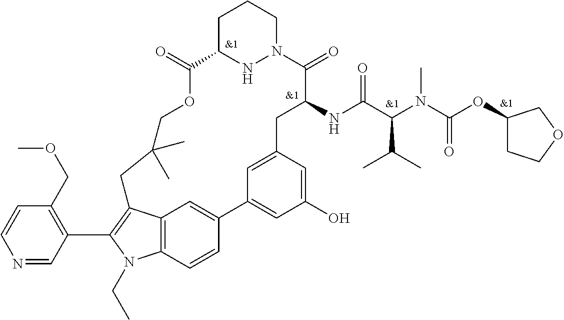 [SMILES: CCn1c(-c2cnccc2COC)c2c3cc(ccc31)-c1cc(O)cc(c1)C[C@H](NC(=O)[C@H](C(C)C)N(C)C(=O)O[C@@H]1CCOC1)C(=O)N1CCC[C@H](N1)C(=O)OCC(C)(C)C2] |
| AA83 | 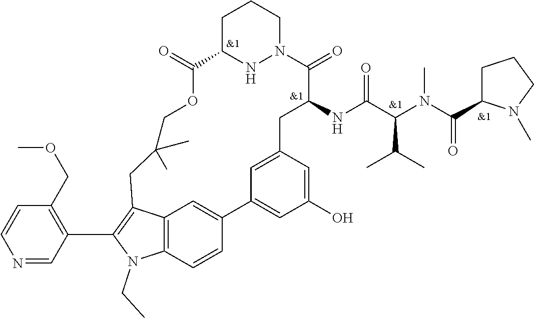 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCCN1C)C(=O)N1CCC[C@H](N1)C(=O)OCC(C)(C)C2] |
| AA84 | 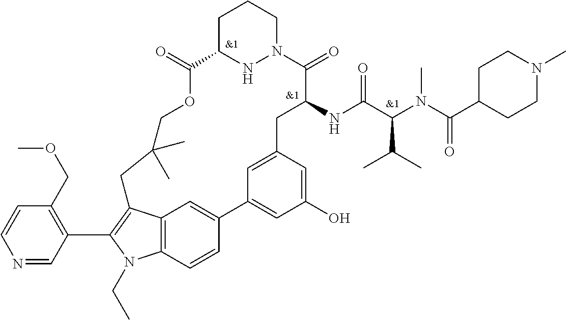 [SMILES: CCn1c(-c2cnccc2COC)c2c3cc(ccc31)-c1cc(O)cc(c1)C[C@H](NC(=O)[C@H](C(C)C)N(C)C(=O)C1CCN(C)CC1)C(=O)N1CCC[C@H](N1)C(=O)OCC(C)(C)C2] |
| AA85 | 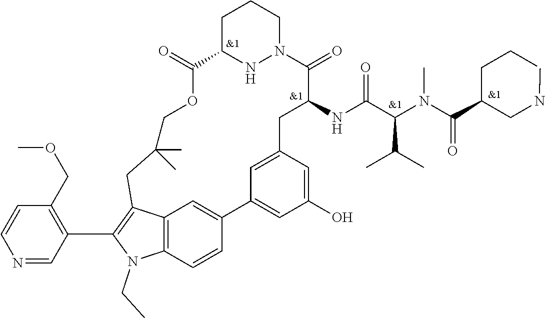 [SMILES: CCC[C@@H](CNC)C(=O)N(C)[C@H](C(=O)N[C@H]1Cc2cc(O)cc(c2)-c2ccc3c(c2)c(c(-c2cnccc2COC)n3CC)CC(C)(C)COC(=O)[C@@H]2CCCN(N2)C1=O)C(C)C] |
| AA86 | 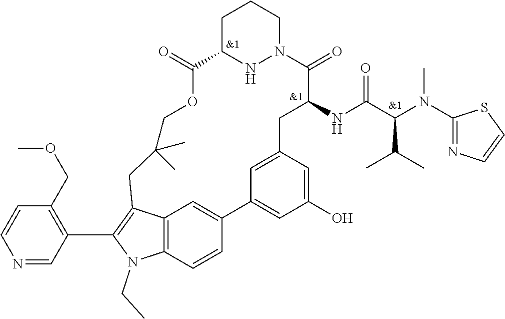 [SMILES: CCn1c(-c2cnccc2COC)c2c3cc(ccc31)-c1cc(O)cc(c1)C[C@H](NC(=O)[C@H](C(C)C)N(C)c1nccs1)C(=O)N1CCC[C@H](N1)C(=O)OCC(C)(C)C2] |
| AA87 | 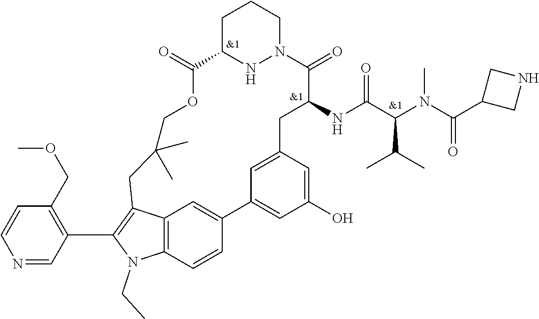 [SMILES: CCn1c(-c2cnccc2COC)c2c3cc(ccc31)-c1cc(O)cc(c1)C[C@H](NC(=O)[C@H](C(C)C)N(C)C(=O)C1CNC1)C(=O)N1CCC[C@H](N1)C(=O)OCC(C)(C)C2] |
| AA88 | 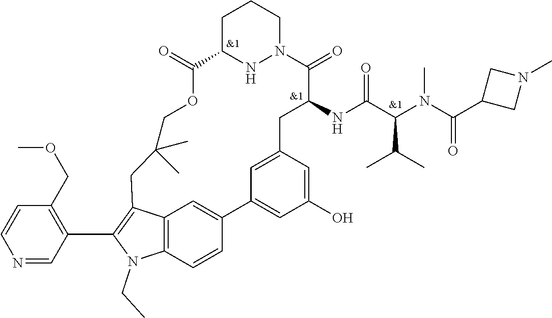 [SMILES: CCn1c(-c2cnccc2COC)c2c3cc(ccc31)-c1cc(O)cc(c1)C[C@H](NC(=O)[C@H](C(C)C)N(C)C(=O)C1CN(C)C1)C(=O)N1CCC[C@H](N1)C(=O)OCC(C)(C)C2] |
| AA89 | 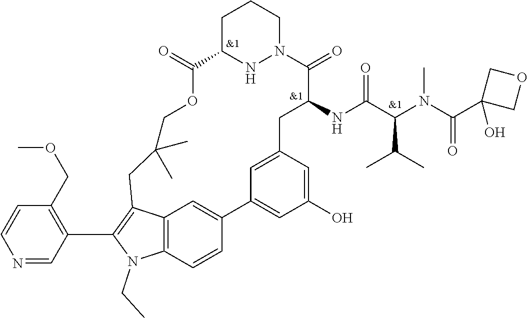 [SMILES: CCn1c(-c2cnccc2COC)c2c3cc(ccc31)-c1cc(O)cc(c1)C[C@H](NC(=O)[C@H](C(C)C)N(C)C(=O)C1(O)COC1)C(=O)N1CCC[C@H](N1)C(=O)OCC(C)(C)C2] |
| AA90 | 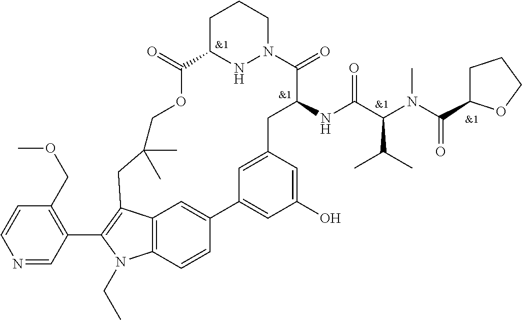 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCCO1)C(=O)N1CCC[C@H](N1)C(=O)OCC(C)(C)C2] |
| AA91 | 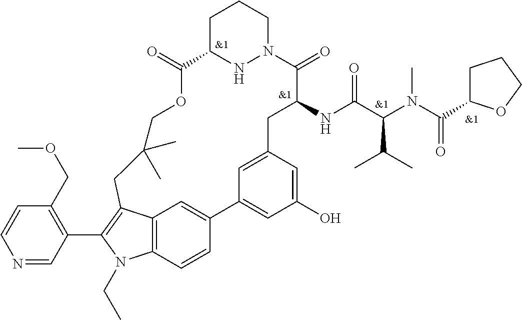 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCO1)C(=O)N1CCC[C@H](N1)C(=O)OCC(C)(C)C2] |
| AA92 | 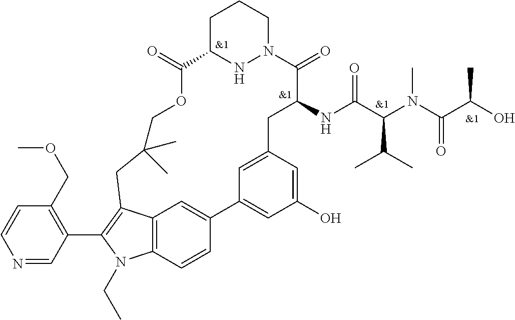 [SMILES: CCn1c(-c2cnccc2COC)c2c3cc(ccc31)-c1cc(O)cc(c1)C[C@H](NC(=O)[C@H](C(C)C)N(C)C(=O)[C@@H](C)O)C(=O)N1CCC[C@H](N1)C(=O)OCC(C)(C)C2] |
| AA93 | 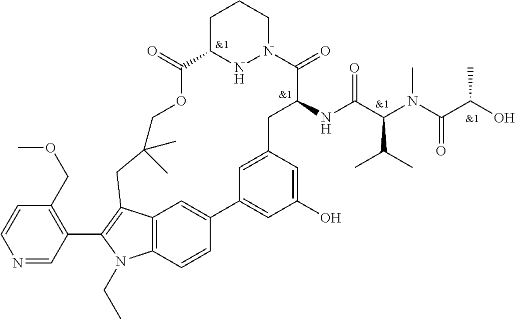 [SMILES: CCn1c(-c2cnccc2COC)c2c3cc(ccc31)-c1cc(O)cc(c1)C[C@H](NC(=O)[C@H](C(C)C)N(C)C(=O)[C@H](C)O)C(=O)N1CCC[C@H](N1)C(=O)OCC(C)(C)C2] |
| AA94 | 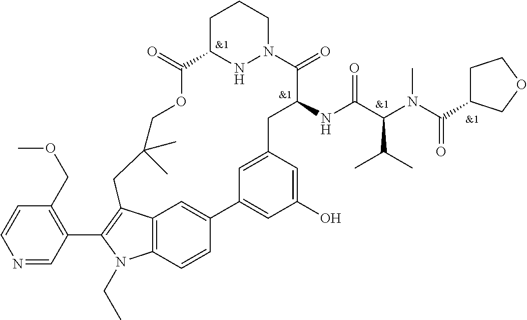 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCOC1)C(=O)N1CCC[C@H](N1)C(=O)OCC(C)(C)C2] |
| AA95 | 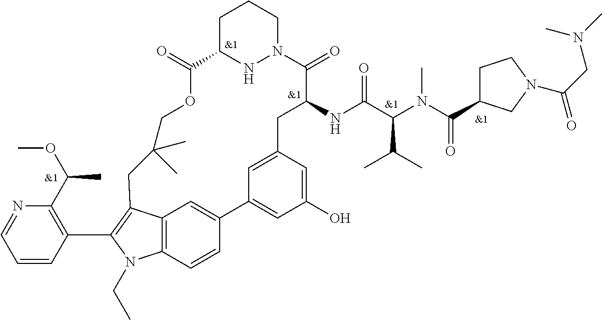 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)CN(C)C)C1)C(=O)N1CCC[C@H](N1)C(=O)OCC(C)(C)C2] |
| AA96 | 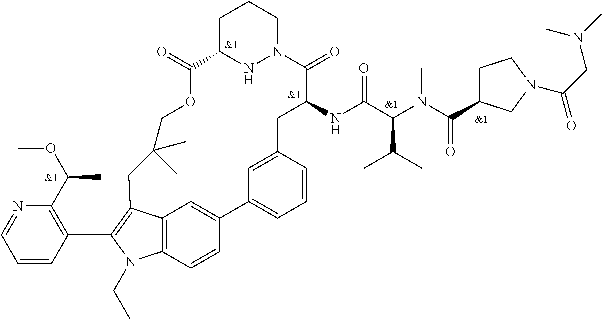 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)CN(C)C)C1)C(=O)N1CCC[C@H](N1)C(=O)OCC(C)(C)C2] |
| AA97 | 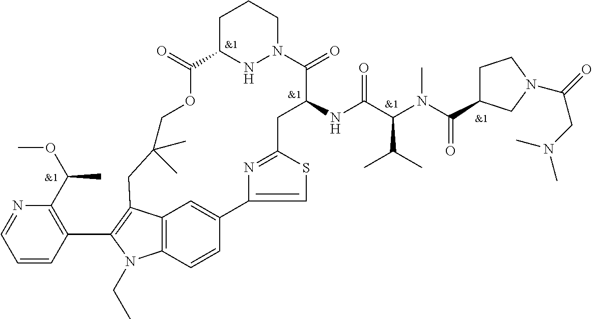 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)CN(C)C)C1)C(=O)N1CCC[C@H](N1)C(=O)OCC(C)(C)C2] |
| AA98 | 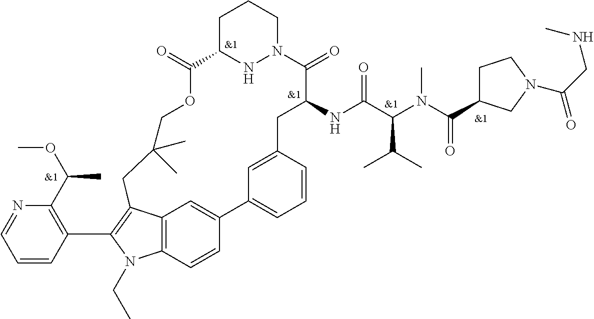 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)CNC)C1)C(=O)N1CCC[C@H](N1)C(=O)OCC(C)(C)C2] |
| AA99 | 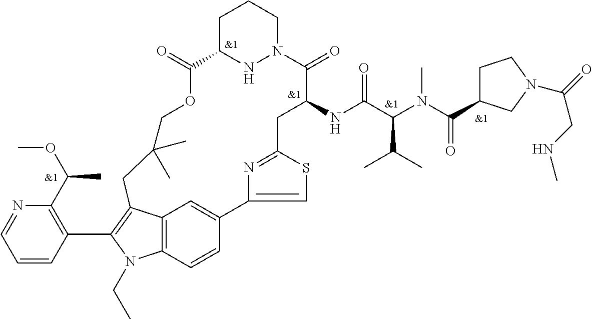 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)CNC)C1)C(=O)N1CCC[C@H](N1)C(=O)OCC(C)(C)C2] |
| AA100 | 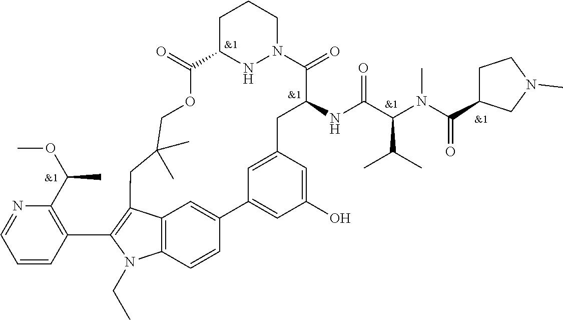 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C)C1)C(=O)N1CCC[C@H](N1)C(=O)OCC(C)(C)C2] |
| AA101 | 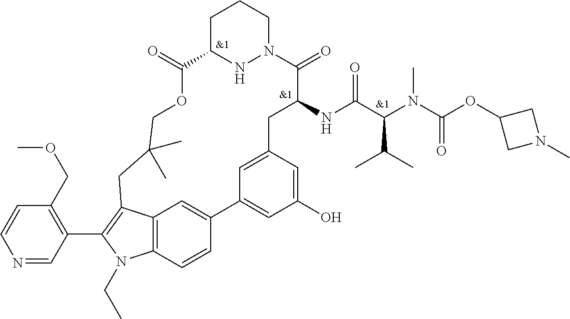 [SMILES: CCn1c(-c2cnccc2COC)c2c3cc(ccc31)-c1cc(O)cc(c1)C[C@H](NC(=O)[C@H](C(C)C)N(C)C(=O)OC1CN(C)C1)C(=O)N1CCC[C@H](N1)C(=O)OCC(C)(C)C2] |
| AA102 | 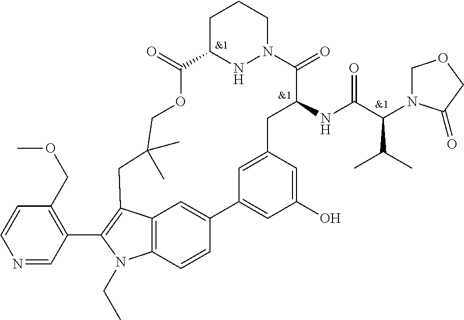 [SMILES: CCn1c(-c2cnccc2COC)c2c3cc(ccc31)-c1cc(O)cc(c1)C[C@H](NC(=O)[C@H](C(C)C)N1COCC1=O)C(=O)N1CCC[C@H](N1)C(=O)OCC(C)(C)C2] |
| AA103 | 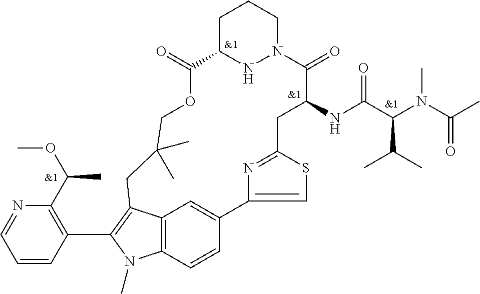 [SMILES: CO[C@@H](C)c1ncccc1-c1c2c3cc(ccc3n1C)-c1csc(n1)C[C@H](NC(=O)[C@H](C(C)C)N(C)C(C)=O)C(=O)N1CCC[C@H](N1)C(=O)OCC(C)(C)C2] |
| AA104 | 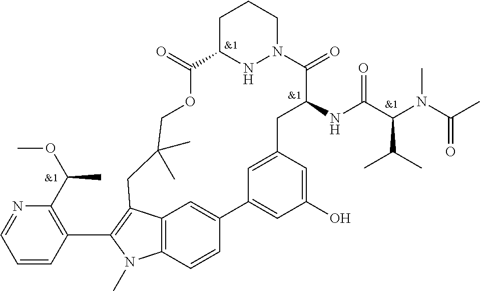 [SMILES: CO[C@@H](C)c1ncccc1-c1c2c3cc(ccc3n1C)-c1cc(O)cc(c1)C[C@H](NC(=O)[C@H](C(C)C)N(C)C(C)=O)C(=O)N1CCC[C@H](N1)C(=O)OCC(C)(C)C2] |
| AA105 | 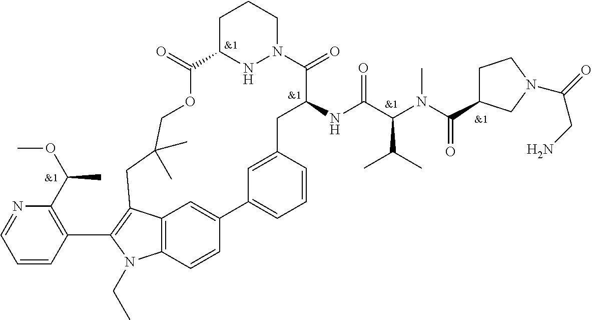 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)CN)C1)C(=O)N1CCC[C@H](N1)C(=O)OCC(C)(C)C2] |
| AA106 | 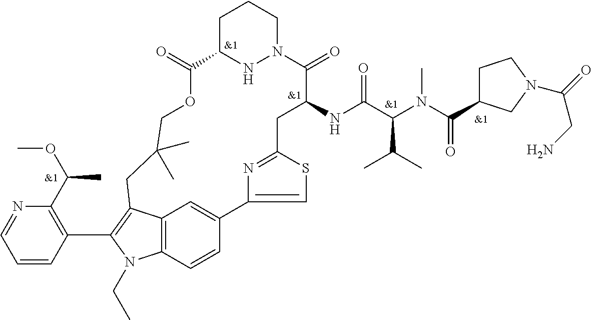 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)CN)C1)C(=O)N1CCC[C@H](N1)C(=O)OCC(C)(C)C2] |
| AA107 | 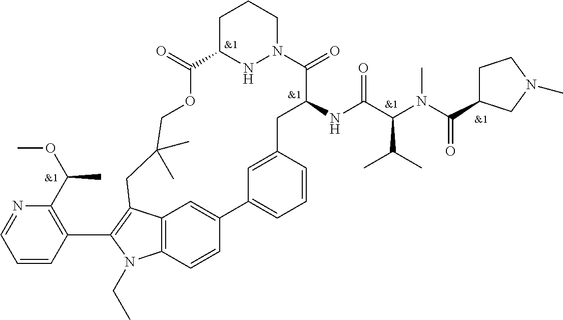 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C)C1)C(=O)N1CCC[C@H](N1)C(=O)OCC(C)(C)C2] |
| AA108 | 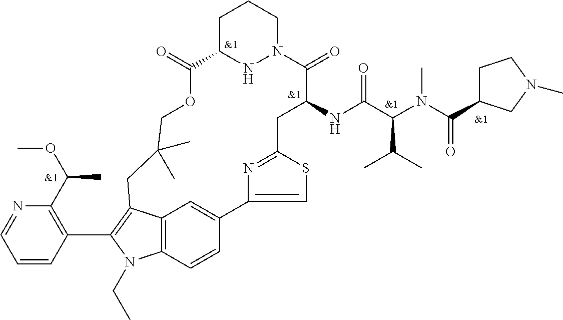 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C)C1)C(=O)N1CCC[C@H](N1)C(=O)OCC(C)(C)C2] |
| AA109 | 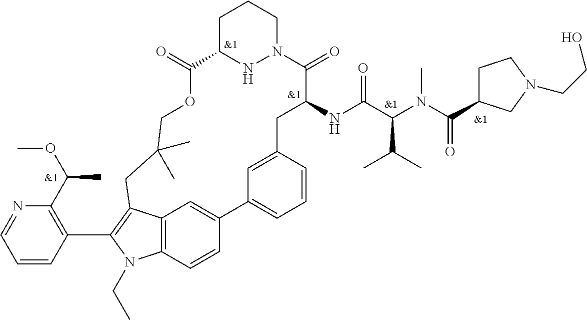 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(CCO)C1)C(=O)N1CCC[C@H](N1)C(=O)OCC(C)(C)C2] |
| AA110 | 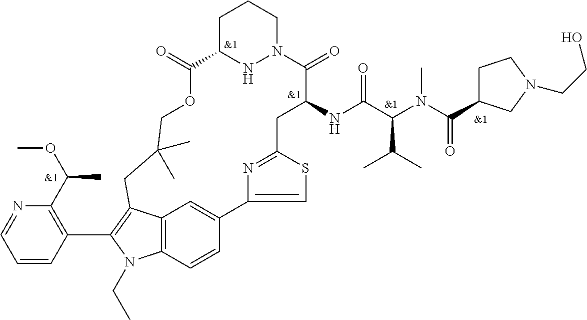 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(CCO)C1)C(=O)N1CCC[C@H](N1)C(=O)OCC(C)(C)C2] |
| AA111 | 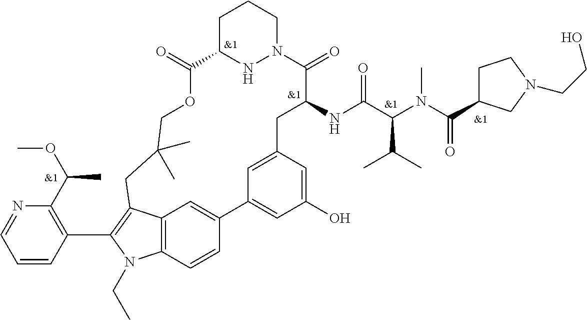 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(CCO)C1)C(=O)N1CCC[C@H](N1)C(=O)OCC(C)(C)C2] |
| AA112 | 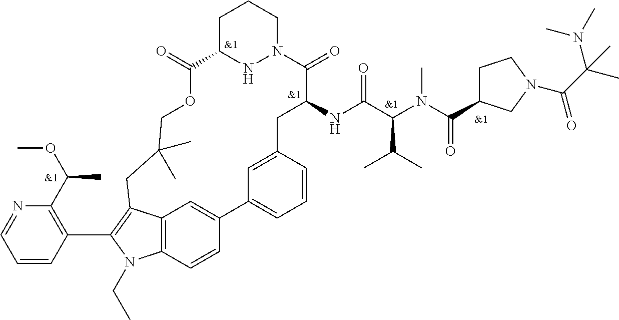 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)C(C)(C)N(C)C)C1)C(=O)N1CCC[C@H](N1)C(=O)OCC(C)(C)C2] |
| AA113 | 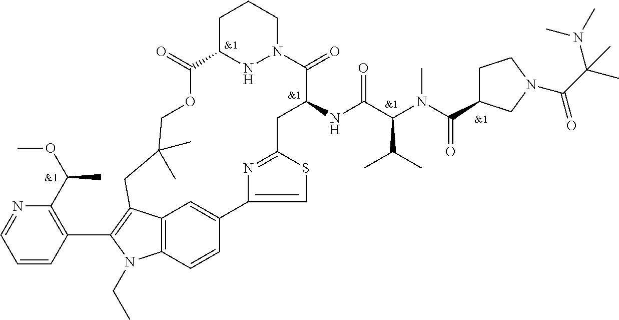 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)C(C)(C)N(C)C)C1)C(=O)N1CCC[C@H](N1)C(=O)OCC(C)(C)C2] |
| AA114 | 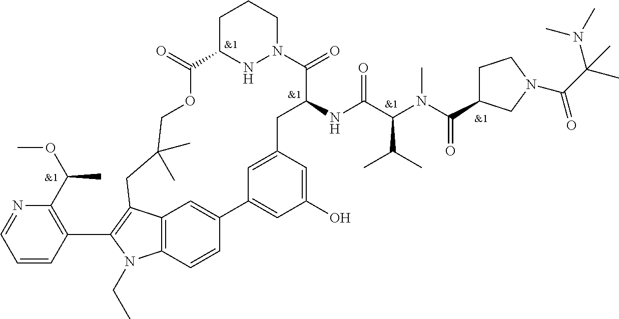 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)C(C)(C)N(C)C)C1)C(=O)N1CCC[C@H](N1)C(=O)OCC(C)(C)C2] |
| AA115 | 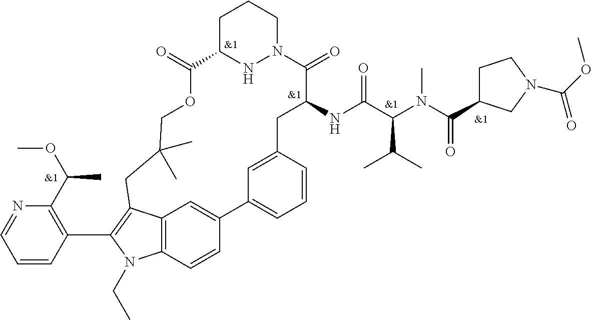 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)OC)C1)C(=O)N1CCC[C@H](N1)C(=O)OCC(C)(C)C2] |
| AA116 | 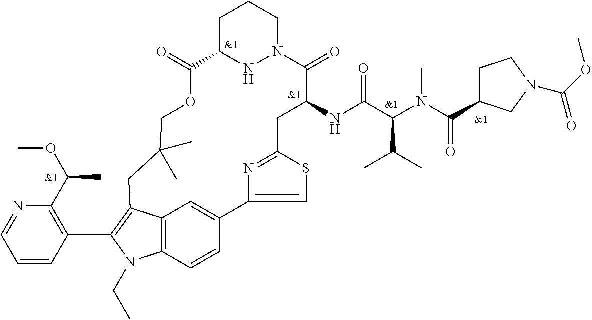 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)OC)C1)C(=O)N1CCC[C@H](N1)C(=O)OCC(C)(C)C2] |
| AA117 | 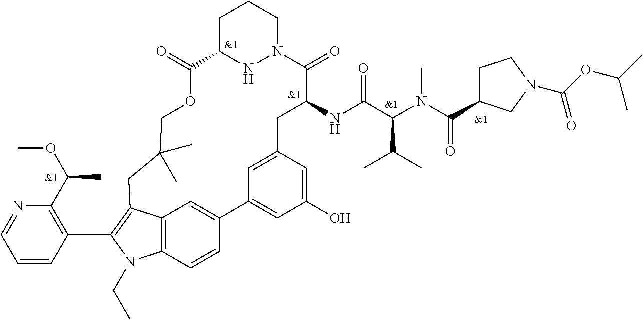 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)OC(C)C)C1)C(=O)N1CCC[C@H](N1)C(=O)OCC(C)(C)C2] |
| AA118 | 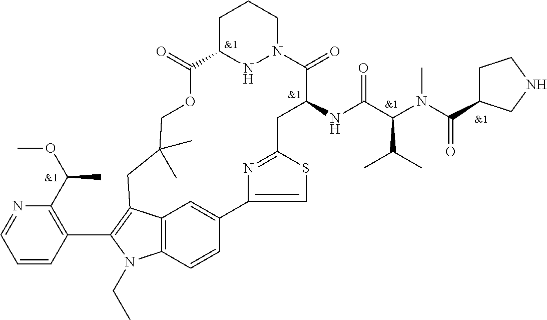 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCNC1)C(=O)N1CCC[C@H](N1)C(=O)OCC(C)(C)C2] |
| AA119 | 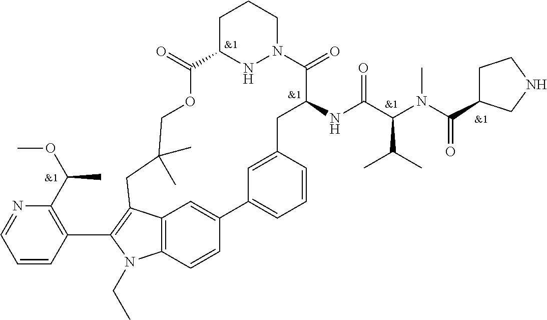 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCNC1)C(=O)N1CCC[C@H](N1)C(=O)OCC(C)(C)C2] |
| AA120 | 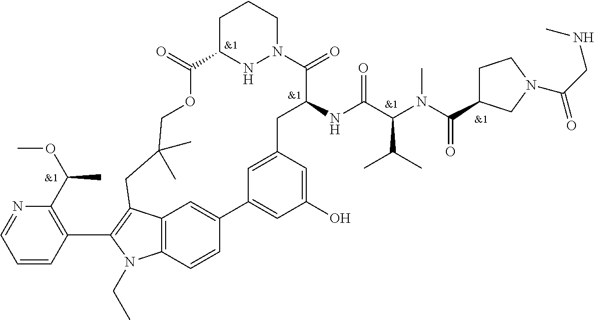 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)CNC)C1)C(=O)N1CCC[C@H](N1)C(=O)OCC(C)(C)C2] |
| AA121 | 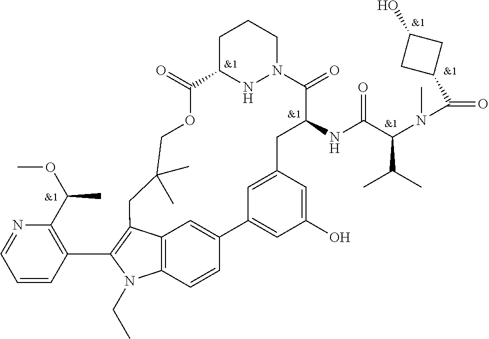 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1C[C@@H](O)C1)C(=O)N1CCC[C@H](N1)C(=O)OCC(C)(C)C2] |
| AA122 | 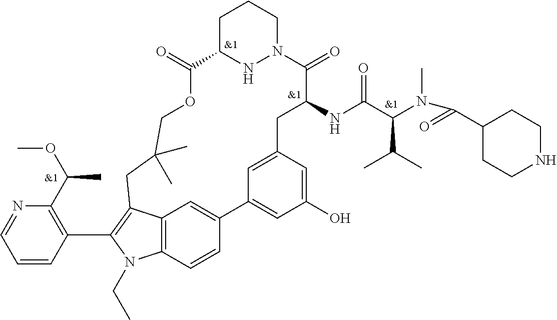 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNCC1)C(=O)N1CCC[C@H](N1)C(=O)OCC(C)(C)C2] |
| AA123 | 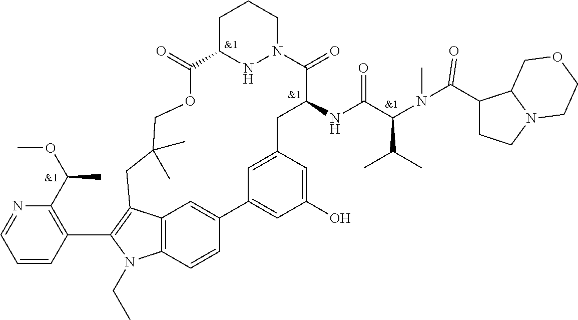 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCN3CCOCC13)C(=O)N1CCC[C@H](N1)C(=O)OCC(C)(C)C2] |
| AA124 | 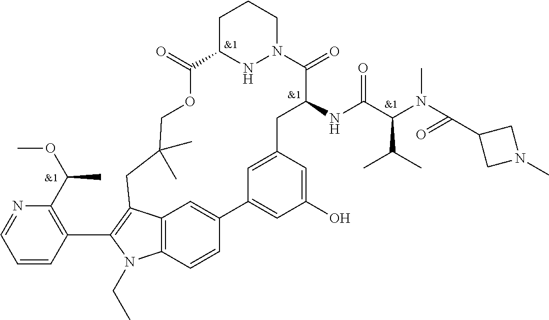 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CN(C)C1)C(=O)N1CCC[C@H](N1)C(=O)OCC(C)(C)C2] |
| AA125 | 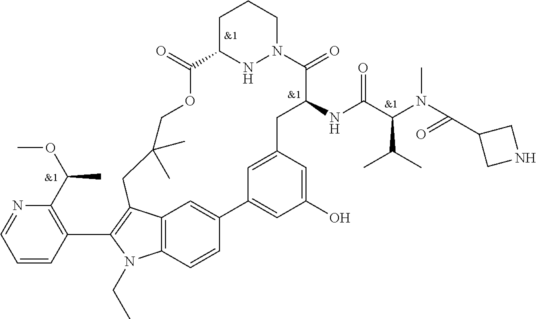 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CNC1)C(=O)N1CCC[C@H](N1)C(=O)OCC(C)(C)C2] |
| AA126 | 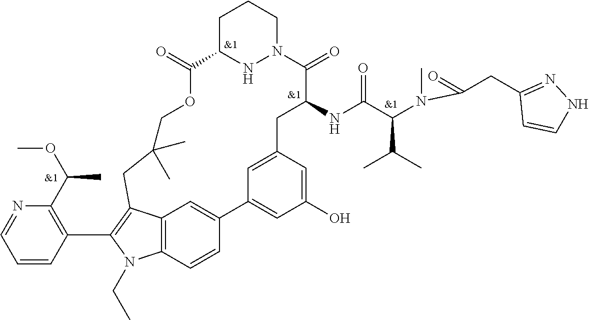 [SMILES: CCn1c(-c2cccnc2[C@H](C)OC)c2c3cc(ccc31)-c1cc(O)cc(c1)C[C@H](NC(=O)[C@H](C(C)C)N(C)C(=O)Cc1cc[nH]n1)C(=O)N1CCC[C@H](N1)C(=O)OCC(C)(C)C2] |
| AA127 | 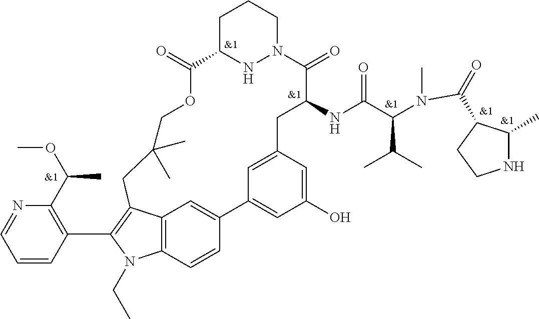 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN[C@H]1C)C(=O)N1CCC[C@H](N1)C(=O)OCC(C)(C)C2] |
| AA128 | 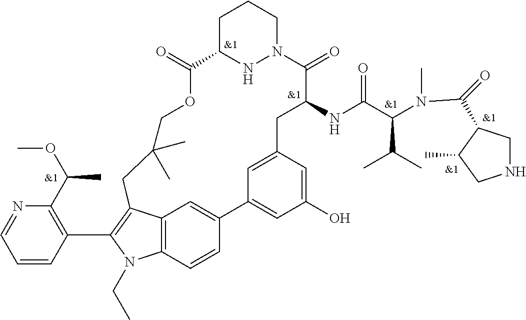 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CNC[C@@H]1C)C(=O)N1CCC[C@H](N1)C(=O)OCC(C)(C)C2] |
| AA129 | 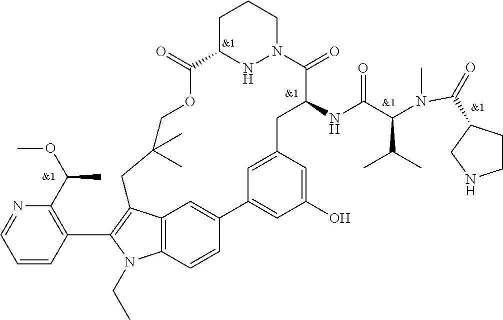 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CCNC1)C(=O)N1CCC[C@H](N1)C(=O)OCC(C)(C)C2] |
| AA130 | 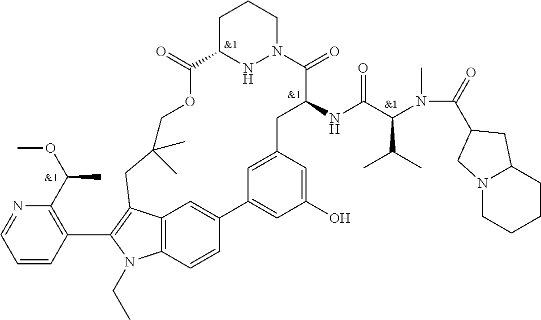 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CC3CCCCN3C1)C(=O)N1CCC[C@H](N1)C(=O)OCC(C)(C)C2] |
| AA131 | 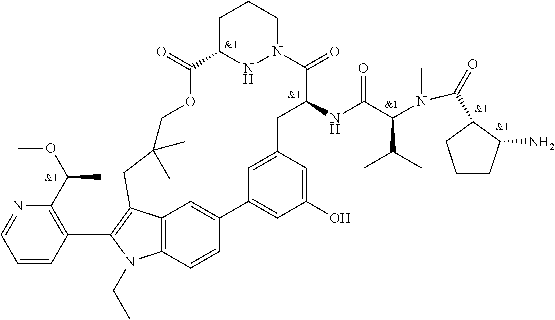 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCC[C@H]1N)C(=O)N1CCC[C@H](N1)C(=O)OCC(C)(C)C2] |
| AA132 | 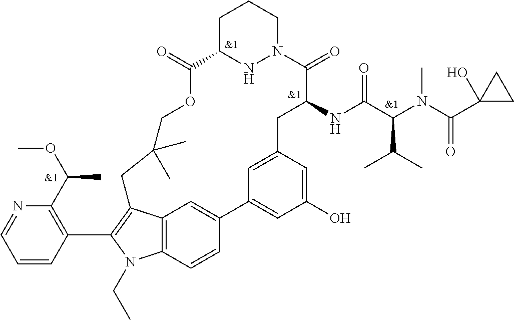 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1(O)CC1)C(=O)N1CCC[C@H](N1)C(=O)OCC(C)(C)C2] |
| AA133 | 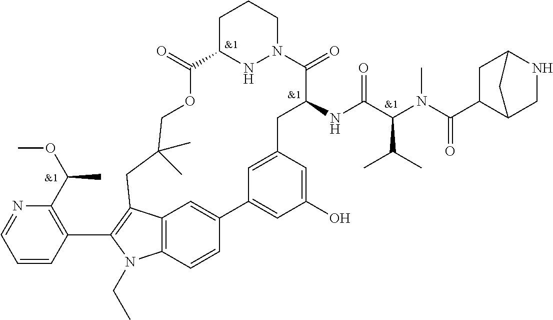 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CC3CC1CN3)C(=O)N1CCC[C@H](N1)C(=O)OCC(C)(C)C2] |
| AA134 | 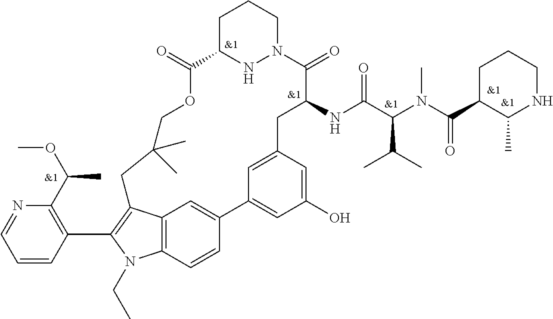 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCCN[C@@H]1C)C(=O)N1CCC[C@H](N1)C(=O)OCC(C)(C)C2] |
| AA135 | 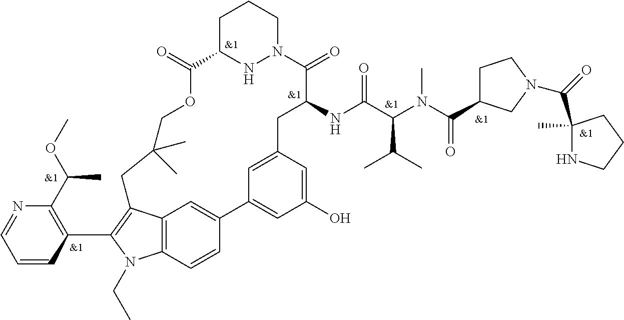 |
| AA136 | 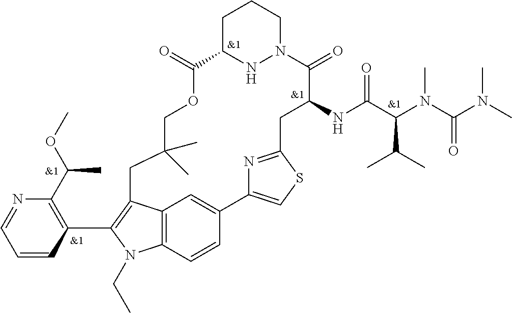 |
| AA137 | 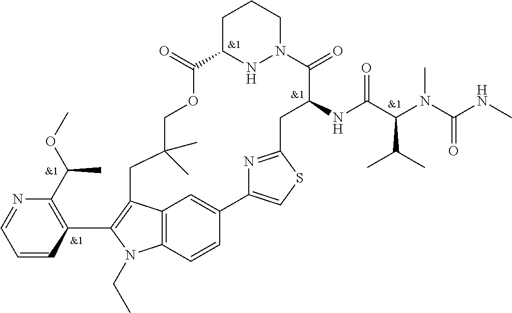 |
| AA138 | 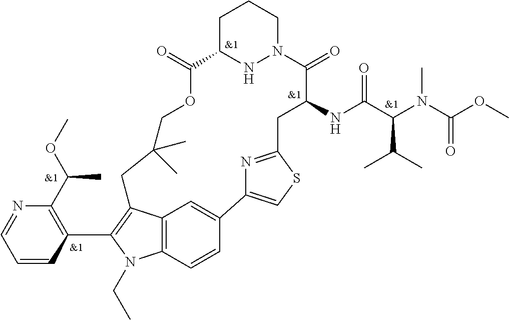 |
| AA139 | 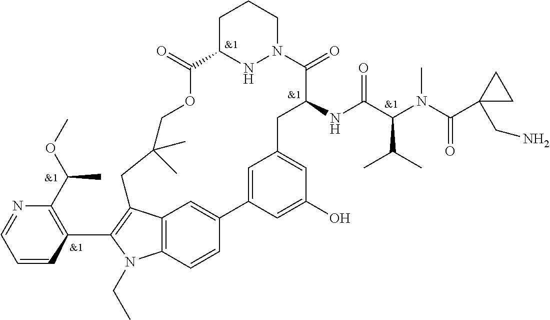 |
| AA140 | 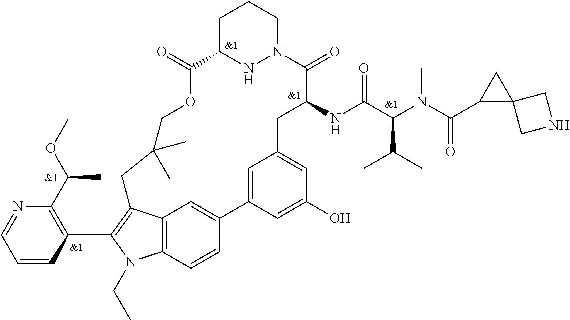 |
| AA141 | 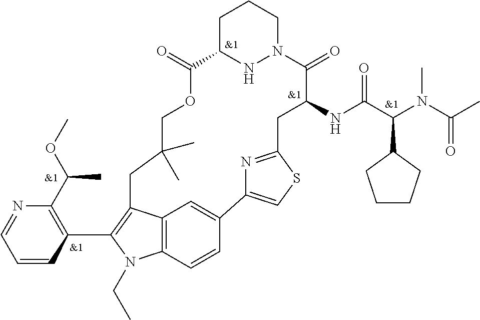 |
| AA142 | 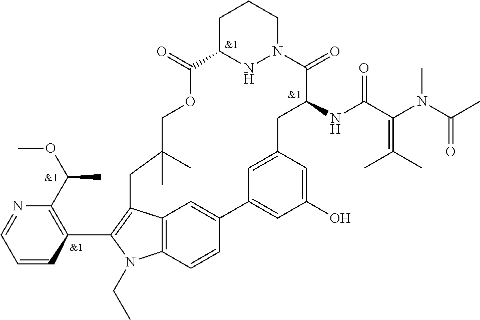 |
| AA143 | 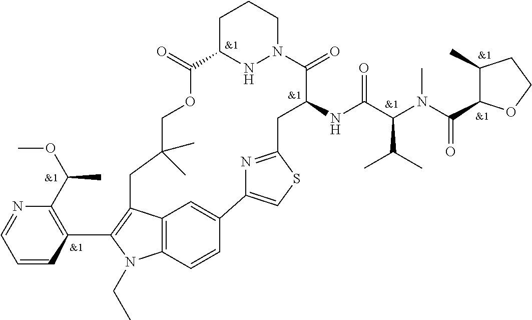 |
| AA144 | 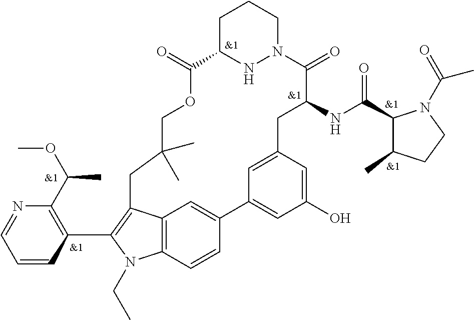 |
| AA145 | 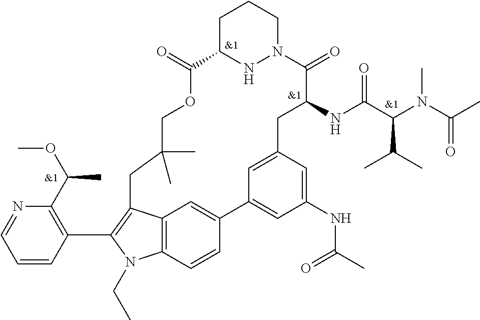 [SMILES: CCn1c(-c2cccnc2[C@H](C)OC)c2c3cc(ccc31)-c1cc(cc(NC(C)=O)c1)C[C@H](NC(=O)[C@H](C(C)C)N(C)C(C)=O)C(=O)N1CCC[C@H](N1)C(=O)OCC(C)(C)C2] |
| AA146 | 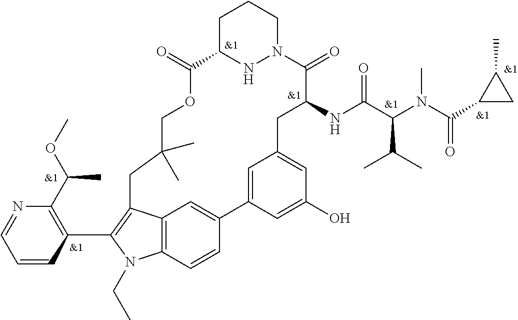 |
| AA147 | 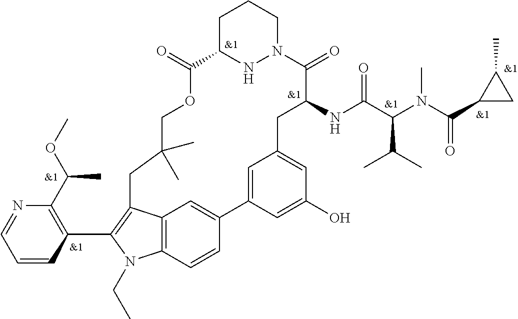 |
| AA148 | 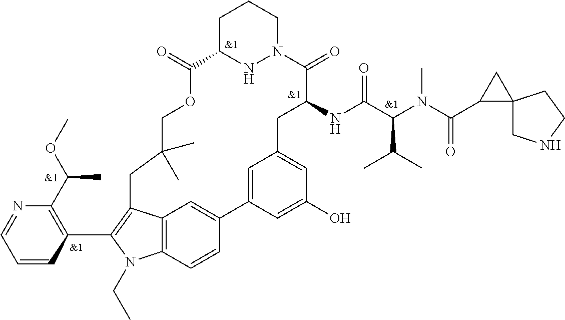 |
| AA149 | 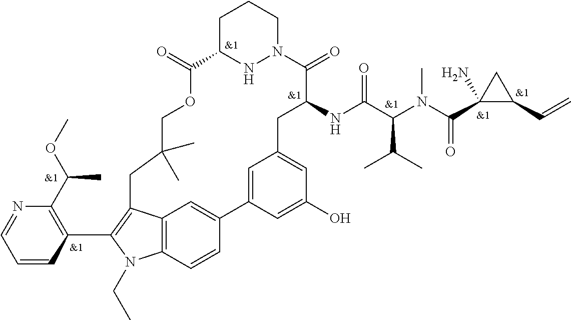 |
| AA150 | 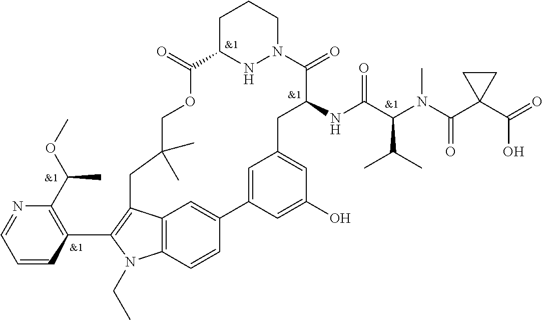 |
| AA151 | 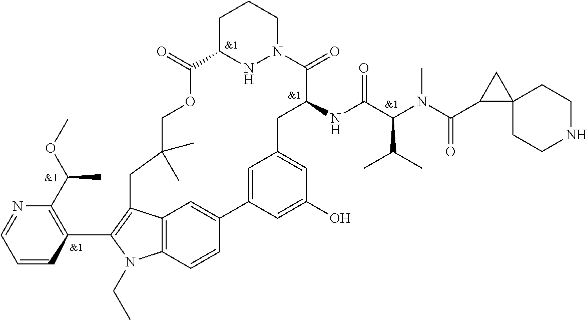 |
| AA152 | 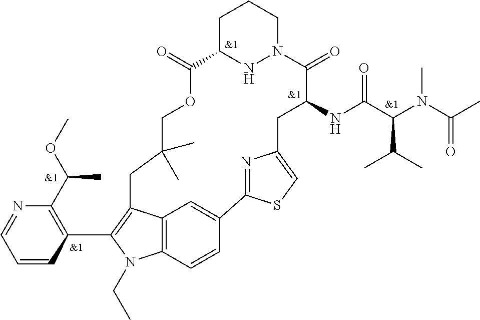 |
| AA153 | 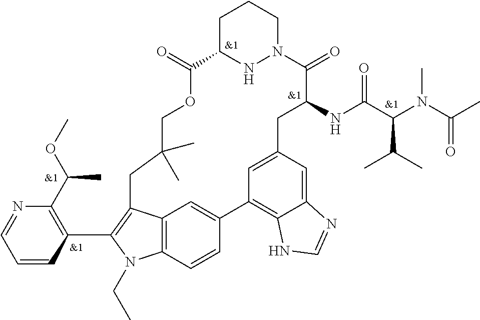 |
| AA154 | 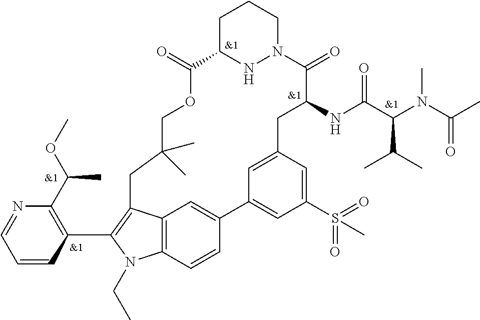 |
| AA155 | 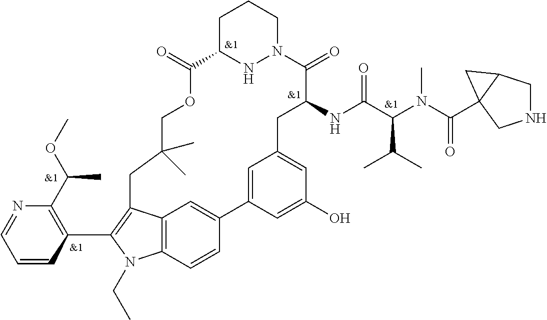 |
| AA156 | 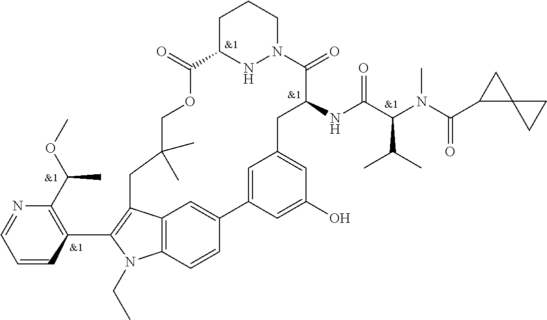 |
| AA157 | 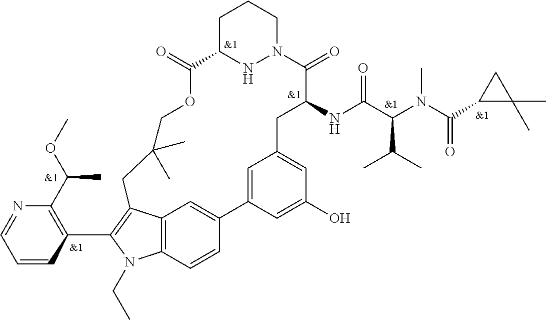 |
| AA158 | 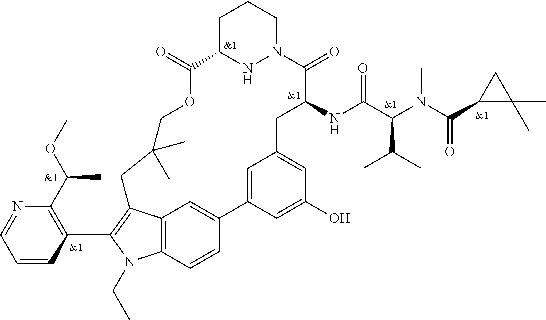 |
| AA159 | 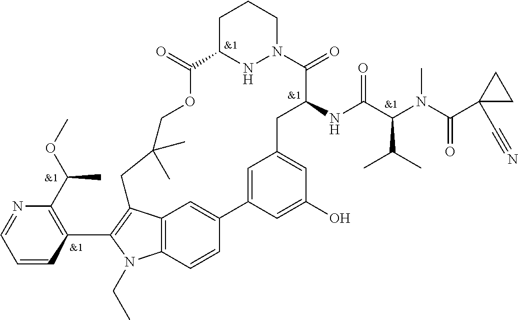 |
| AA160 | 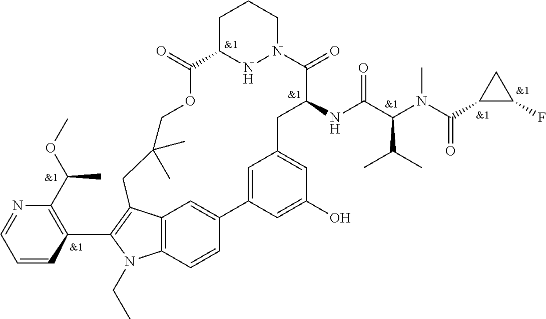 |
| AA161 | 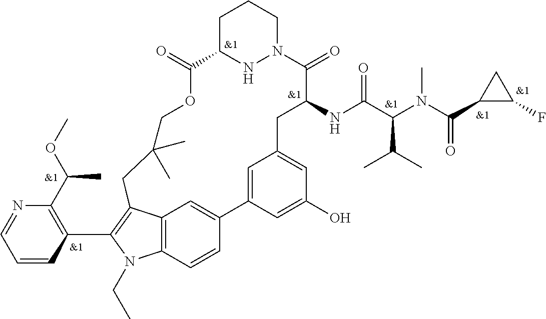 |
| AA162 | 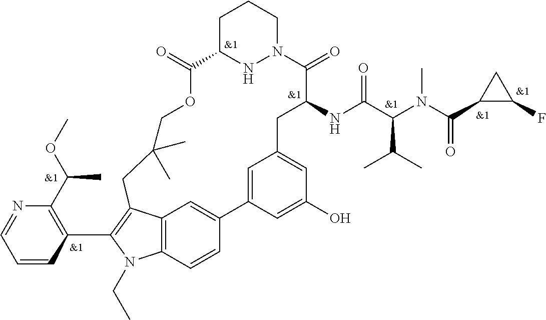 |
| AA163 | 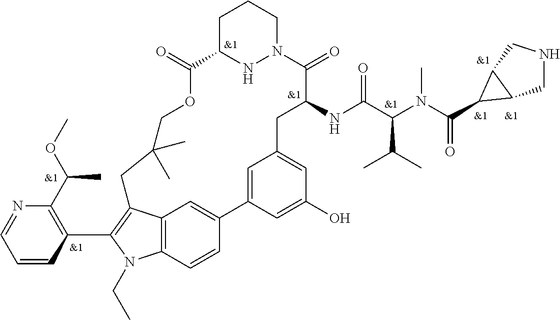 |
| AA164 | 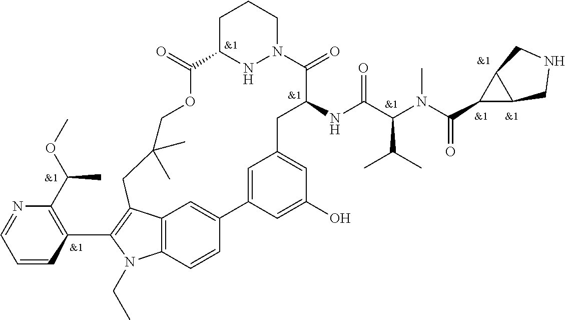 |
| AA165 | 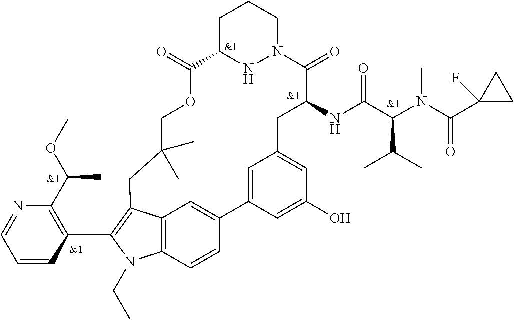 |
| AA166 | 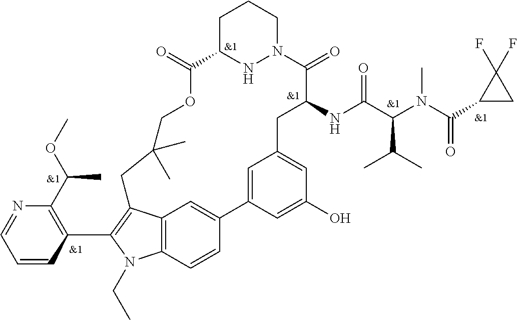 |
| AA167 | 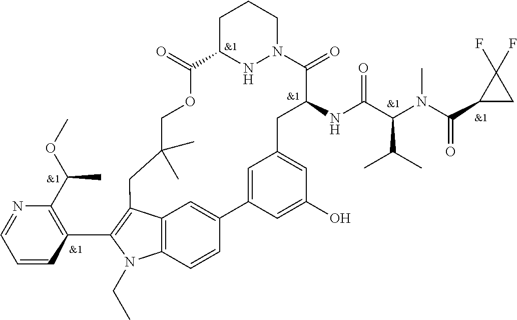 |
| AA168 | 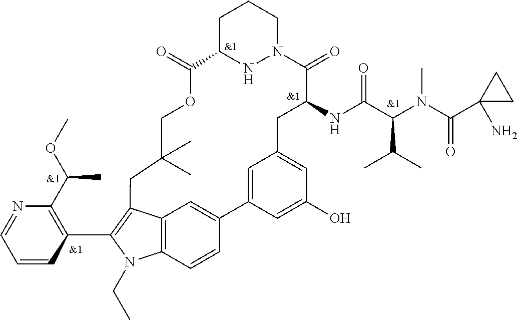 |
| AA169 | 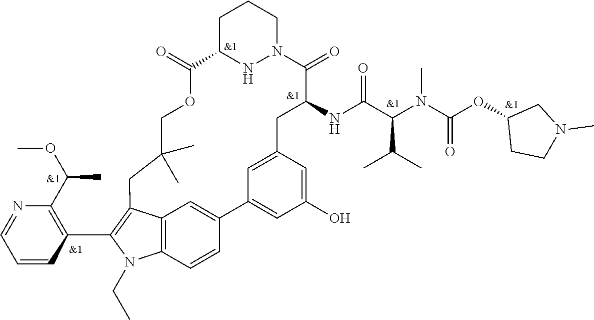 |
| AA170 | 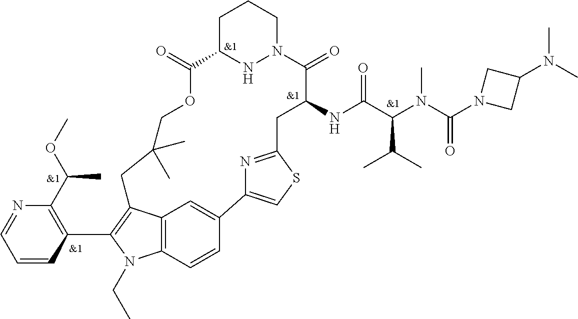 |
| AA171 | 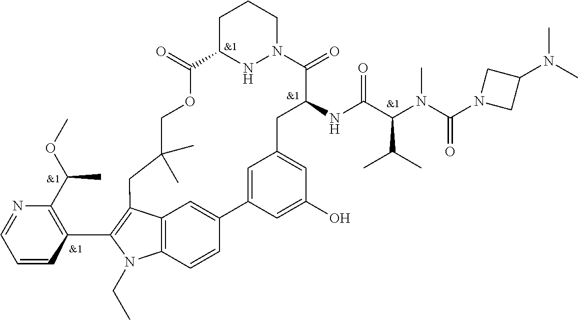 |
| AA172 | 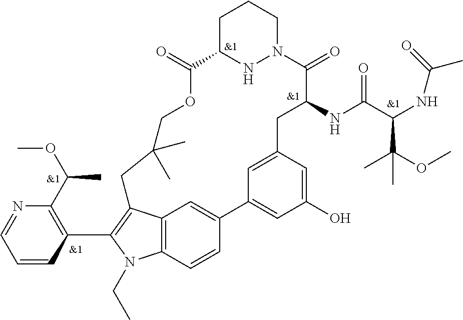 |
| AA173 | 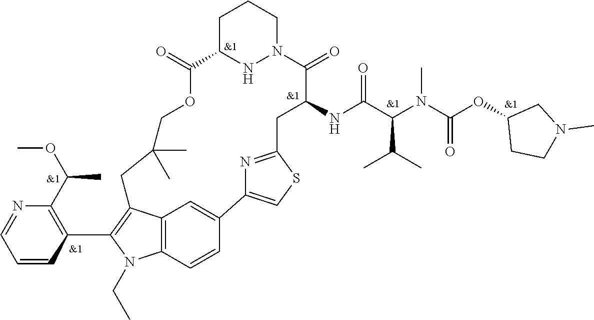 |
| AA174 | 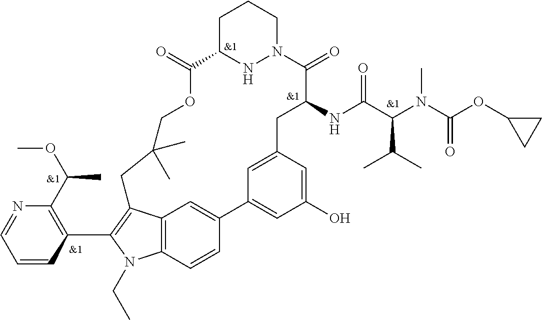 |
| AA175 | 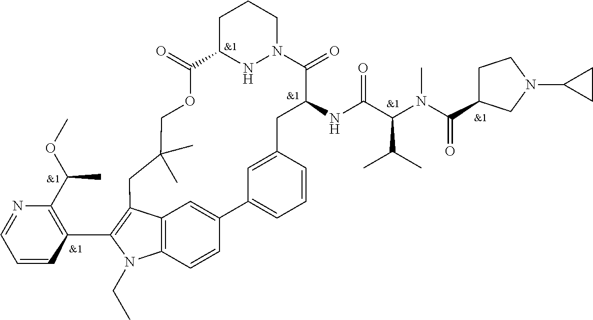 |
| AA176 | 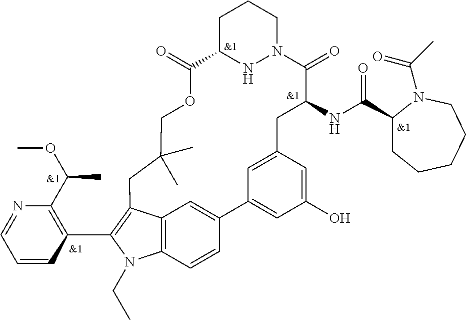 |
| AA177 | 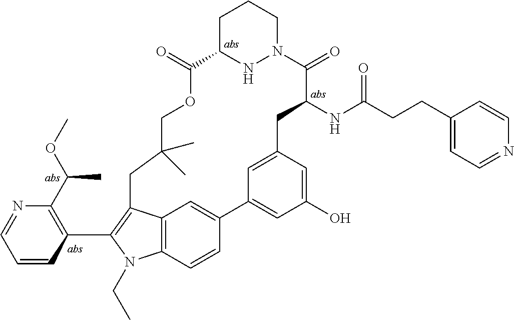 |
| AA178 | 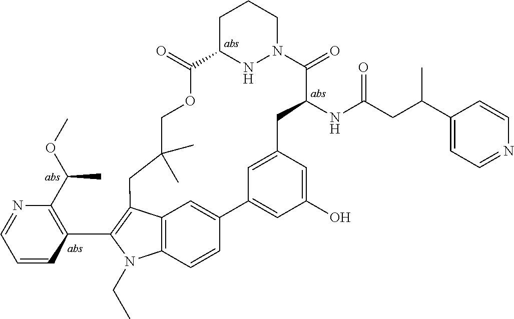 |
| AA179 | 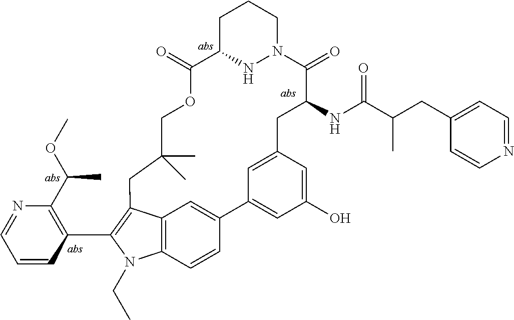 |
| AA180 | 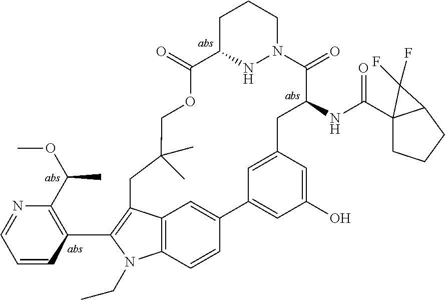 |
| AA181 | 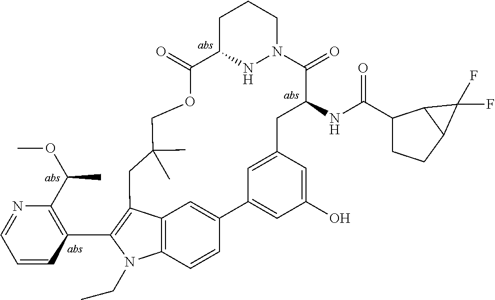 |
| AA182 | 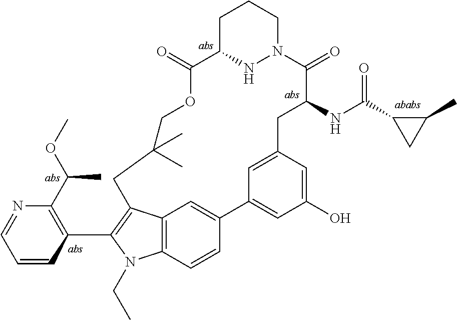 |
| AA183 | 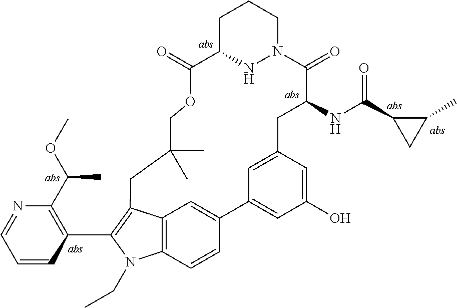 |
| AA184 | 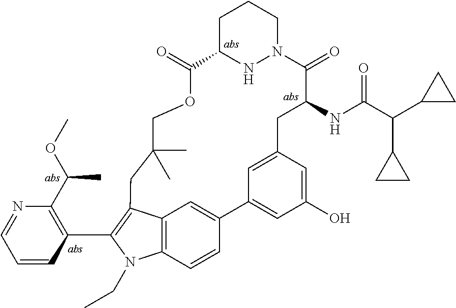 |
| AA185 | 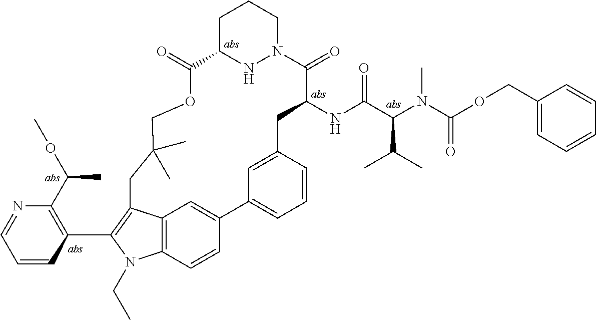 |
| AA186 | 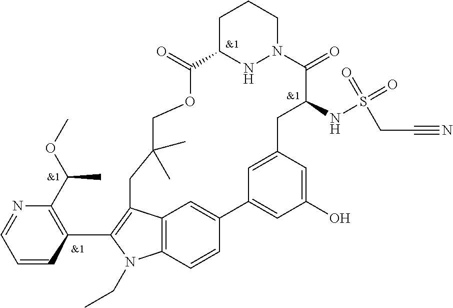 |
| AA187 | 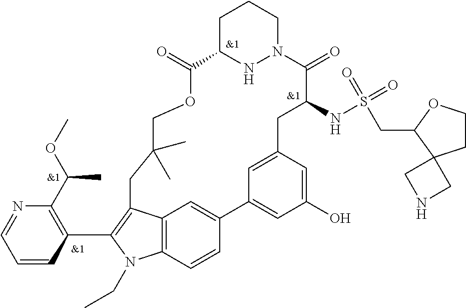 |
| AA188 | 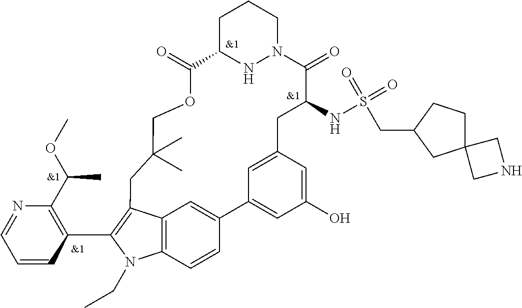 |
| AA189 | 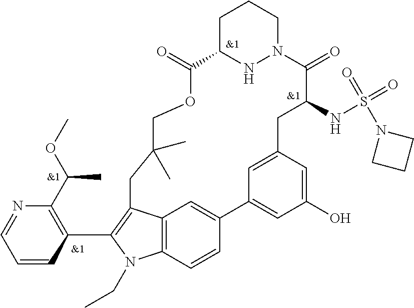 |
| AA190 | 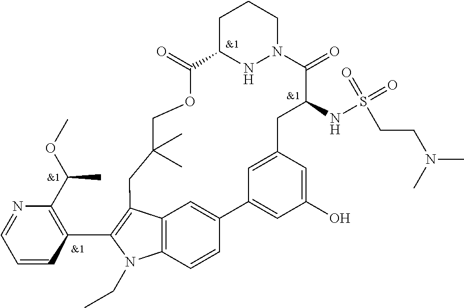 |
| AA191 | 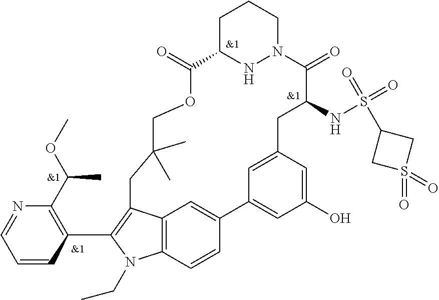 |
| AA192 | 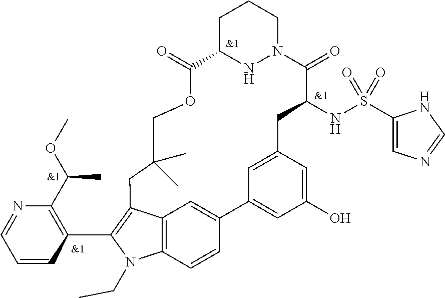 |
| AA193 | 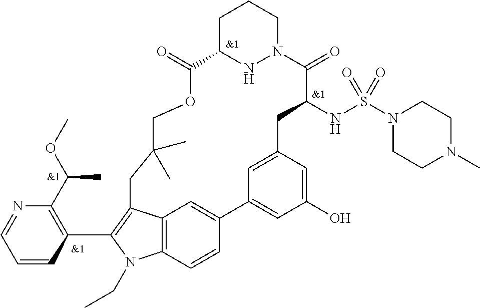 |
| AA194 | 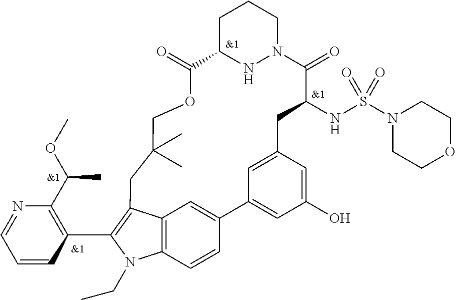 |
| AA195 | 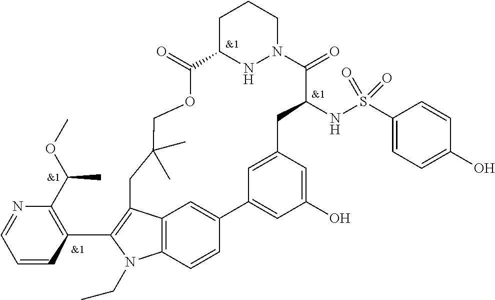 |
| AA196 | 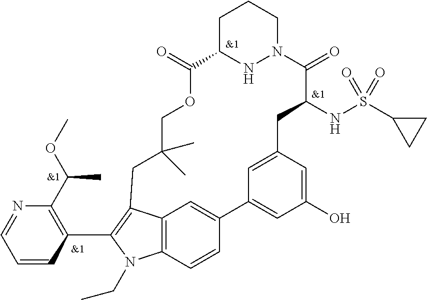 |
| AA197 | 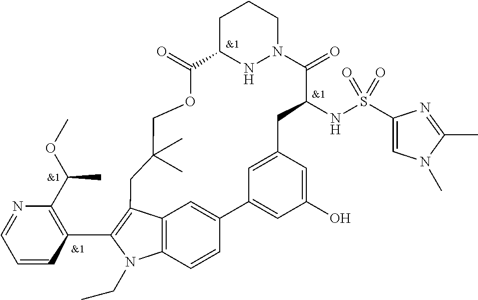 |
| AA198 | 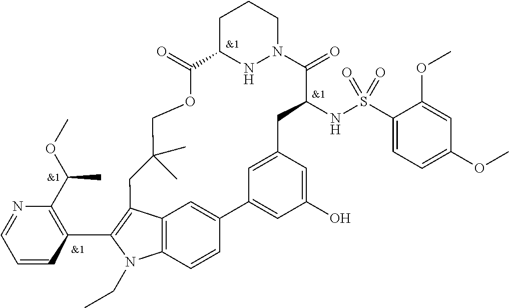 |
| AA199 | 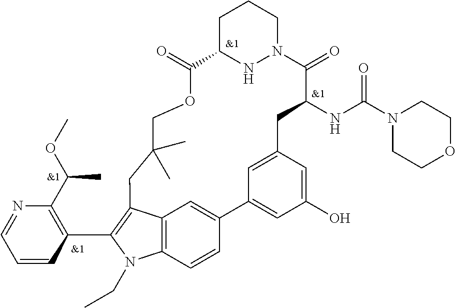 |
| AA200 | 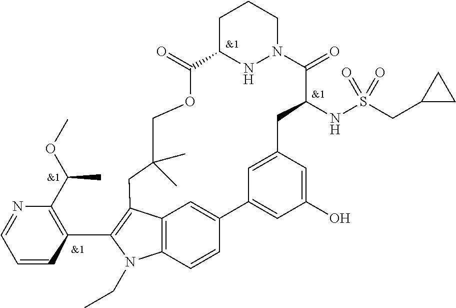 |
| AA201 | 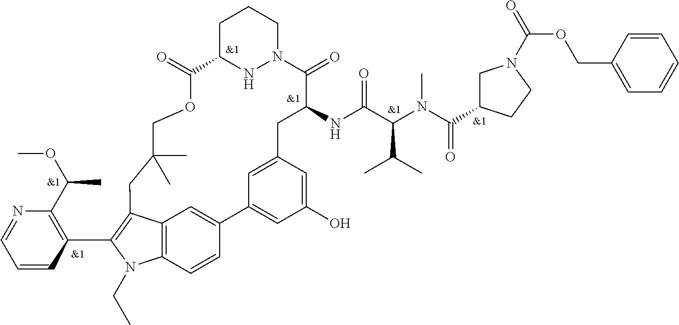 |
| AA202 | 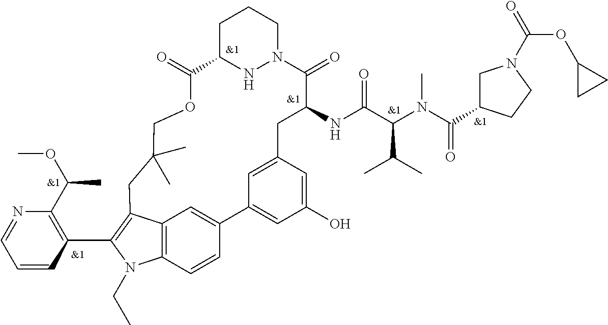 |
| AA203 | 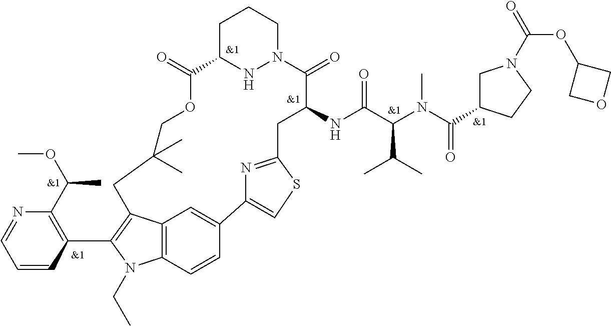 |
| AA204 | 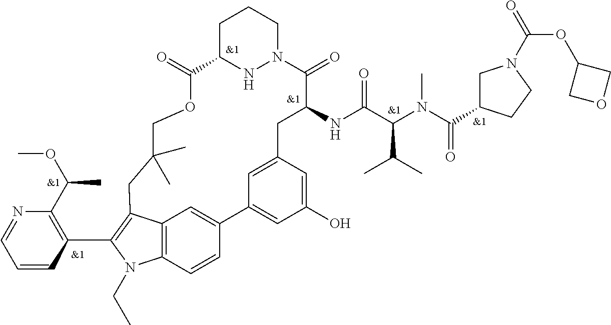 |
| AA205 | 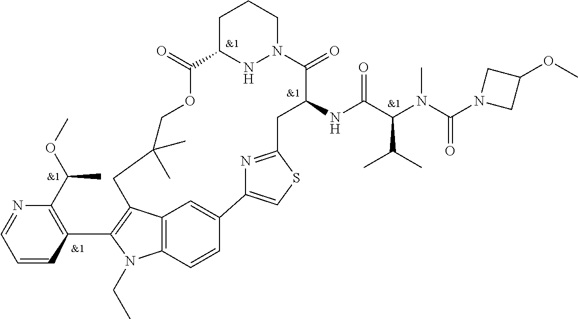 |
| AA206 | 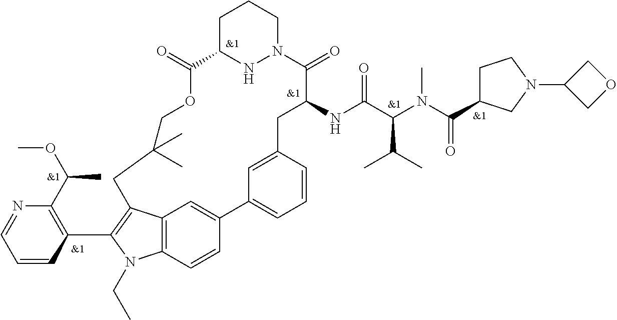 |
| AA207 | 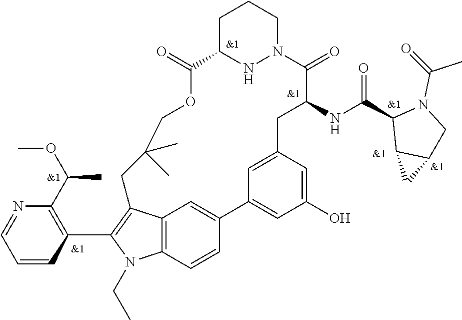 |
| AA208 | 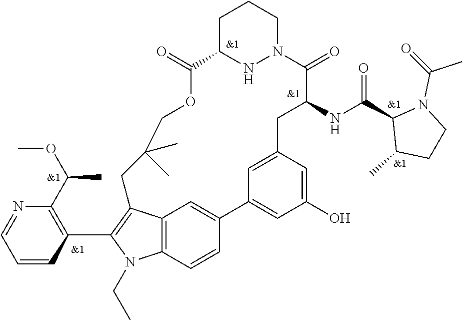 |
| AA209 | 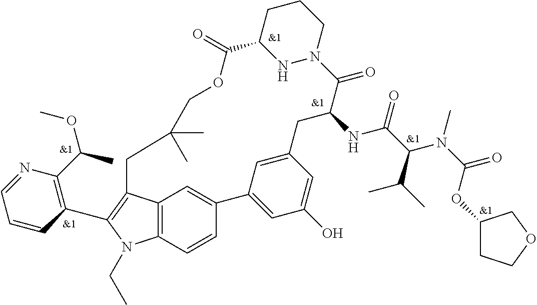 |
| AA210 | 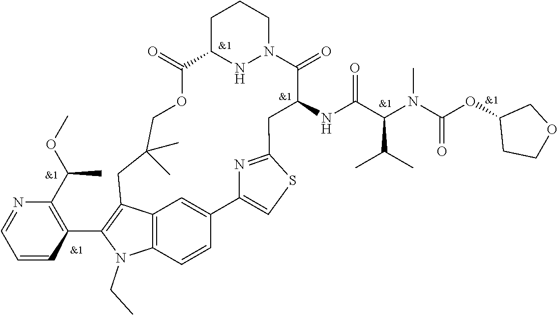 |
| AA211 | 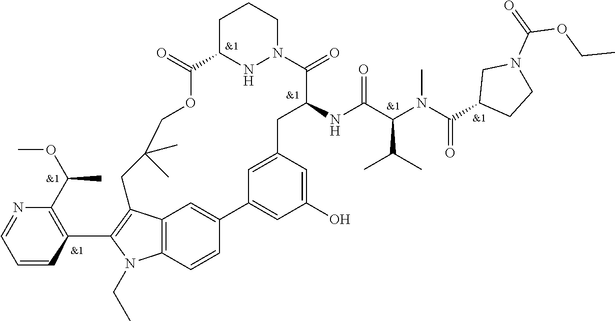 |
| AA212 | 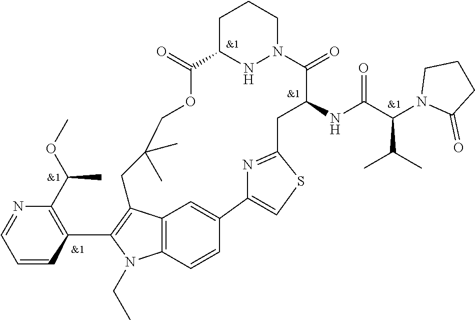 |
| AA213 | 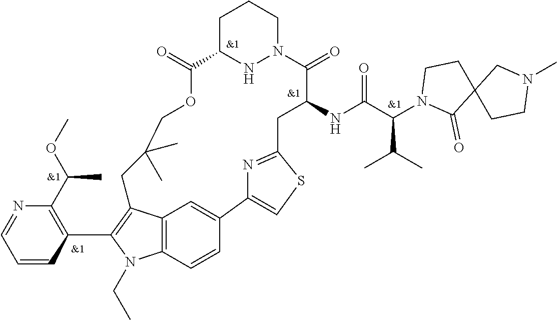 |
| AA214 | 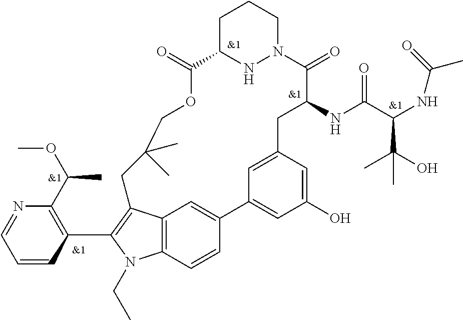 |
| AA215 | 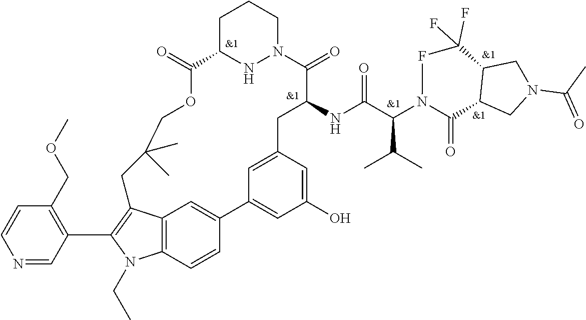 |
| AA216 | 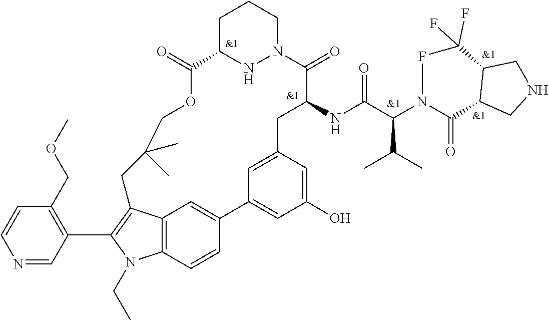 |
| AA217 | 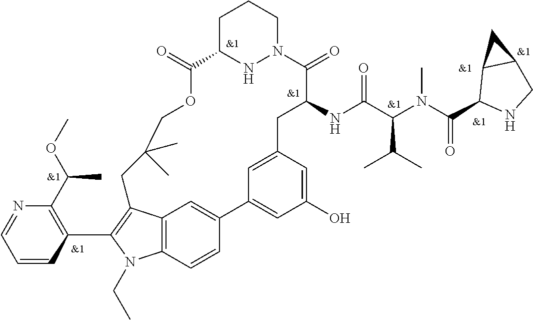 |
| AA218 | 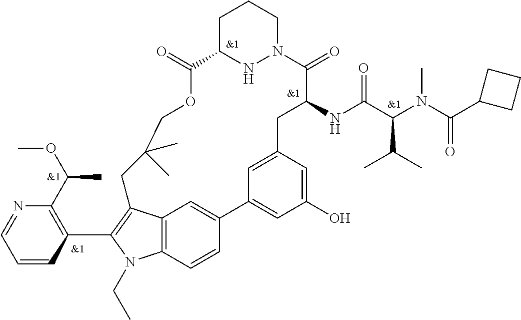 |
| AA219 | 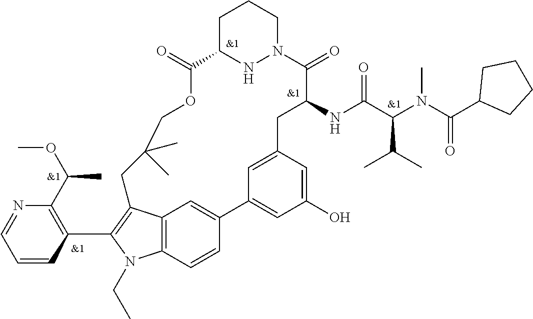 |
| AA220 | 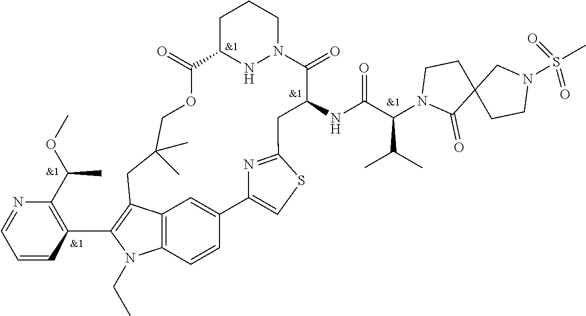 |
| AA221 | 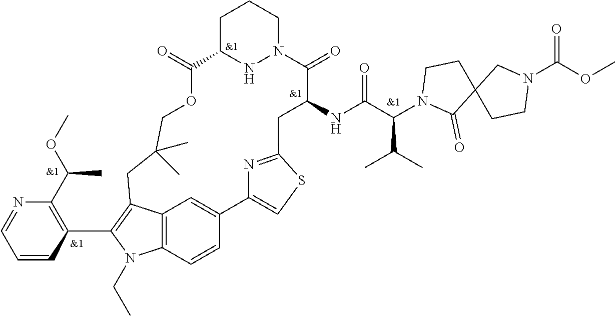 |
| AA222 | 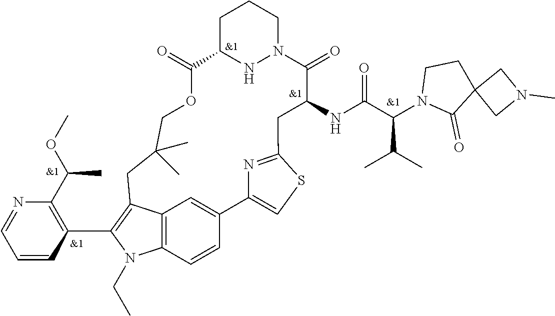 |
| AA223 | 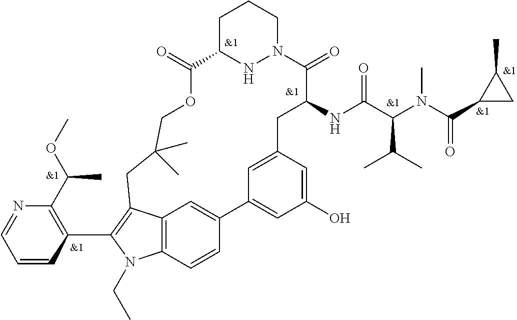 |
| AA224 | 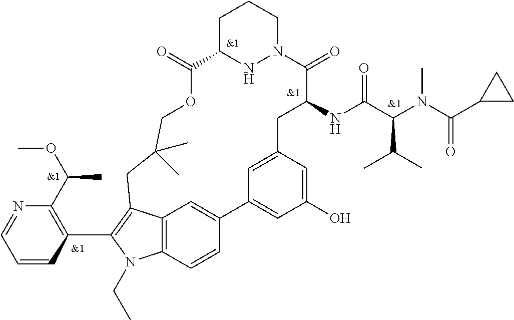 |
| AA225 | 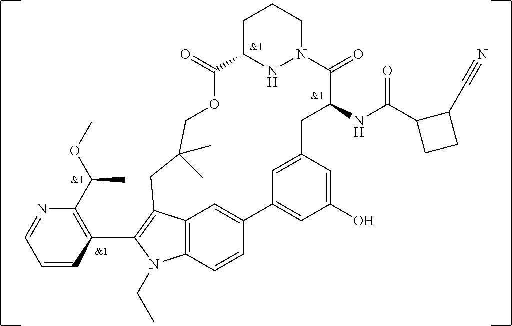 |
| AA226 | 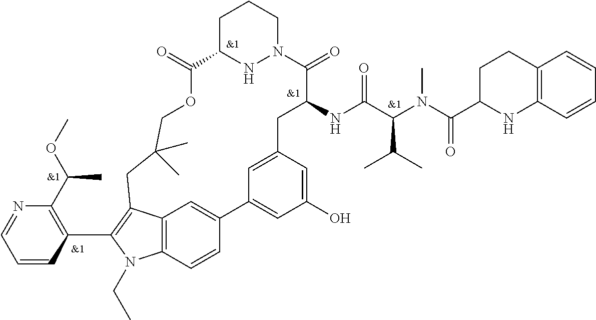 |
| AA227 | 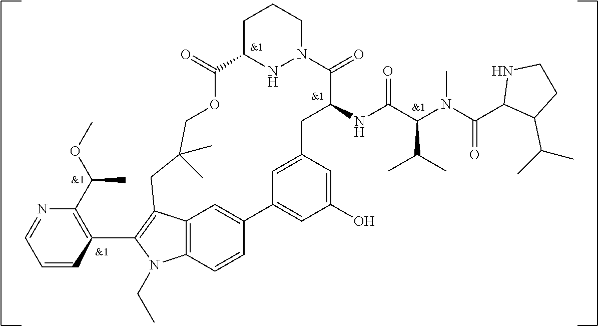 |
| AA228 | 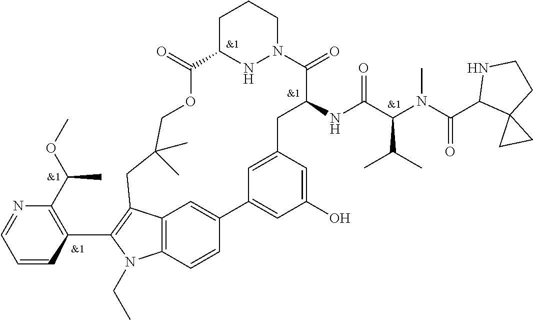 |
| AA229 | 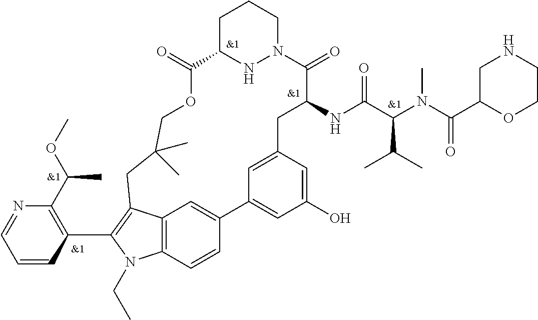 |
| AA230 | 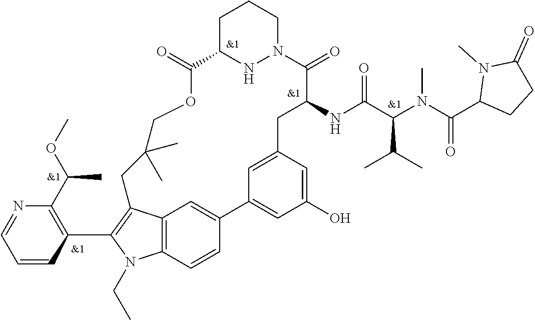 |
| AA231 | 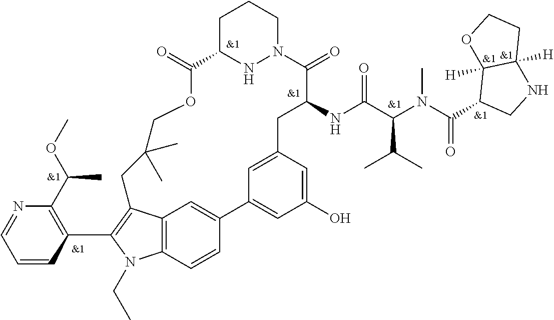 |
| AA232 | 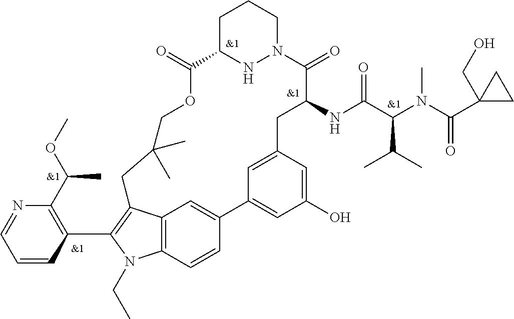 |
| AA233 | 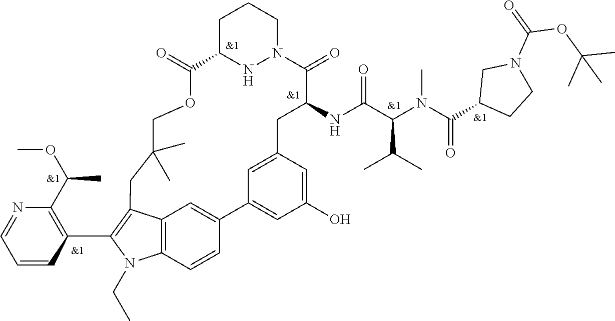 |
| AA234 | 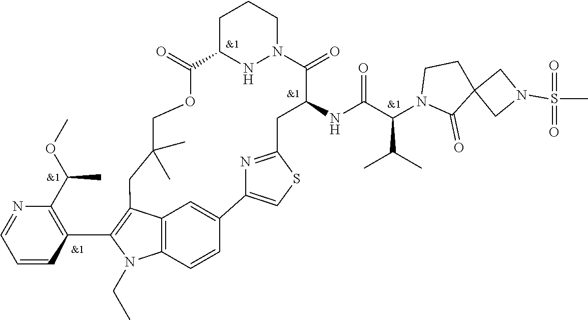 |
| AA235 | 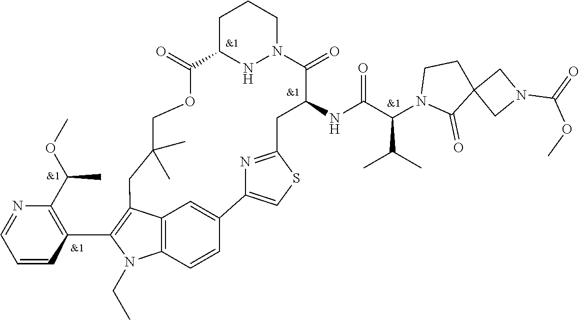 |
| AA236 | 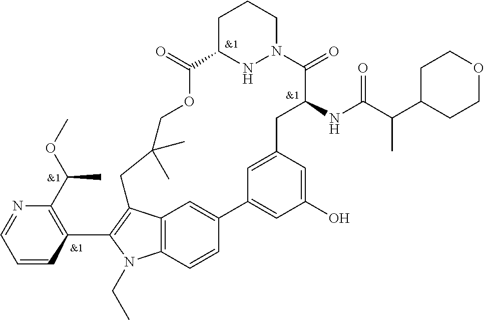 |
| AA237 | 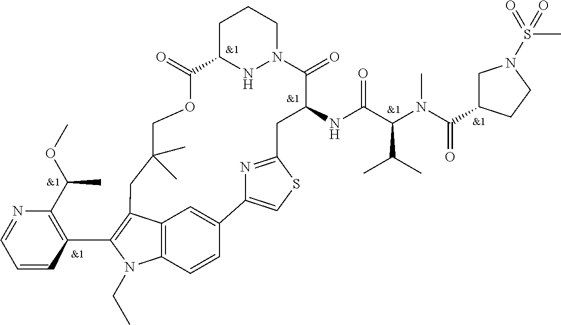 |
| AA238 | 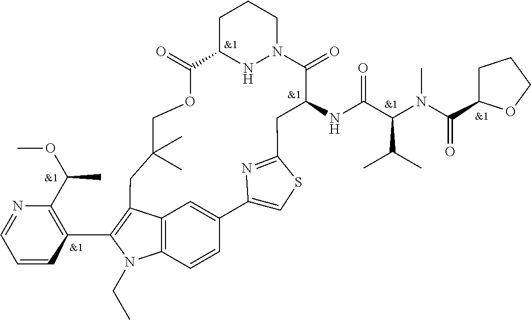 |
| AA239 | 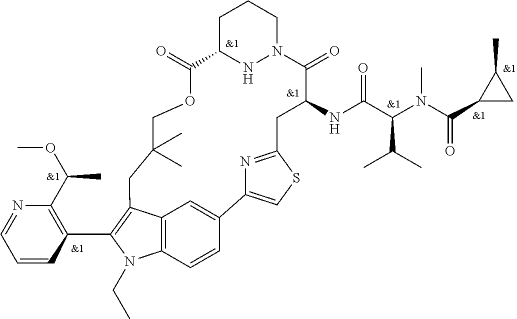 |
| AA240 | 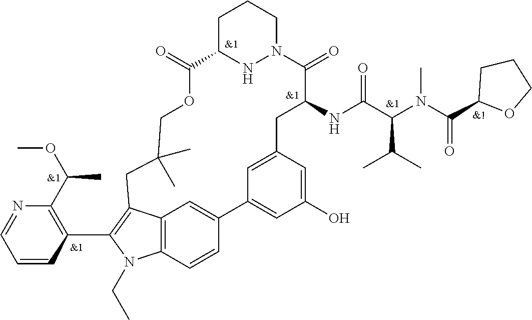 |
| AA241 | 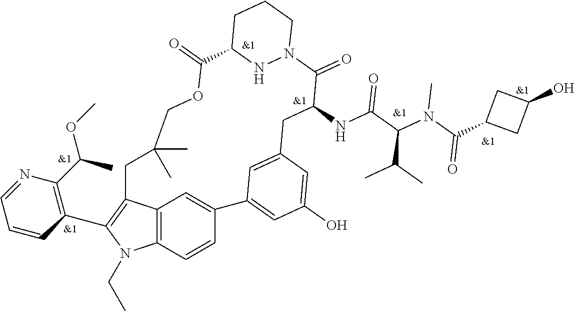 |
| AA242 | 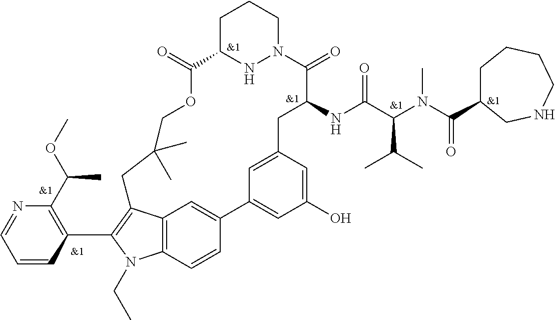 |
| AA243 | 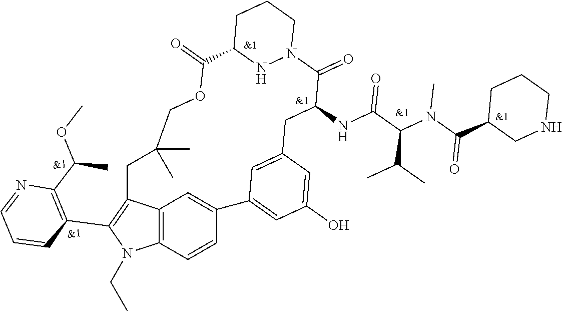 |
| AA244 | 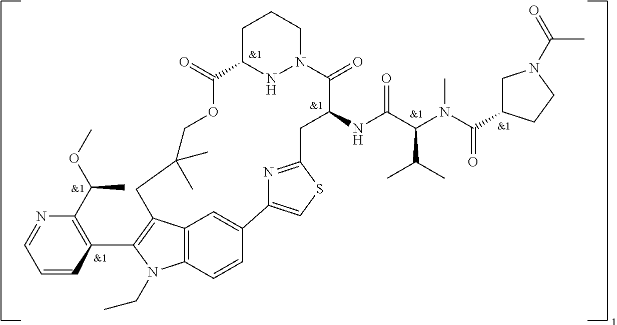 |
| AA245 | 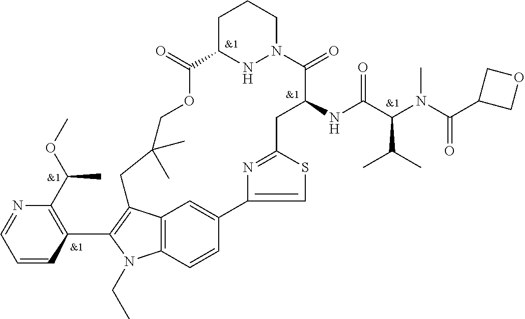 |
| AA246 | 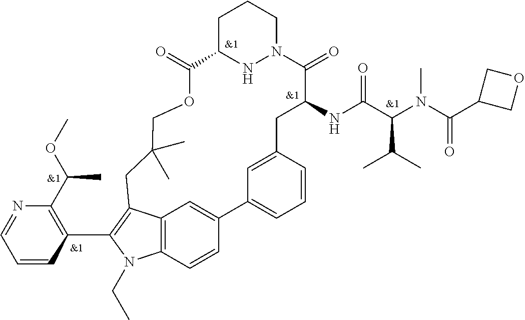 |
| AA247 | 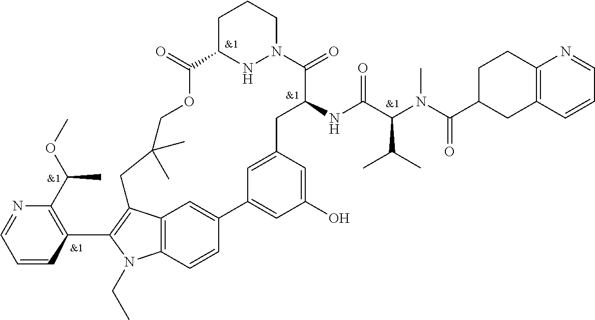 |
| AA248 | 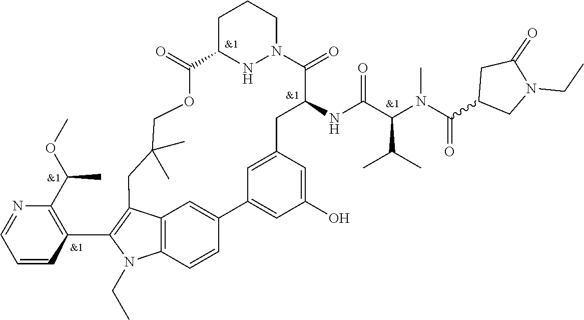 |
| AA249 | 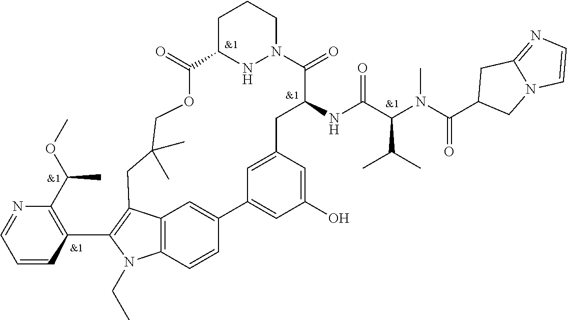 |
| AA250 | 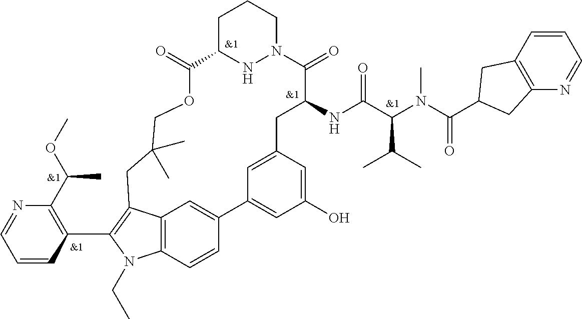 |
| AA251 | 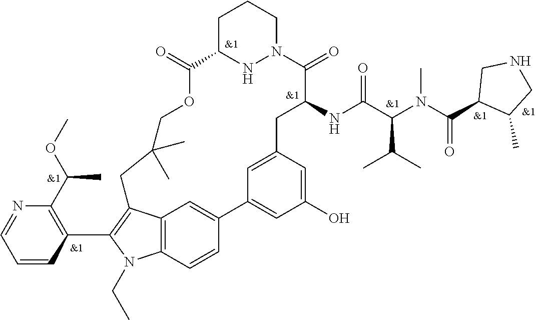 |
| AA252 | 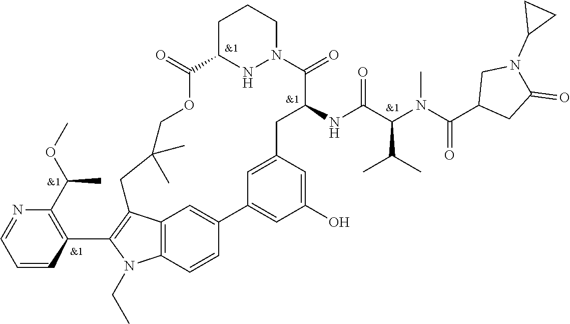 |
| AA253 | 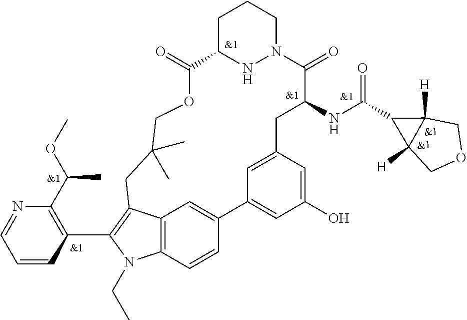 |
| AA254 | 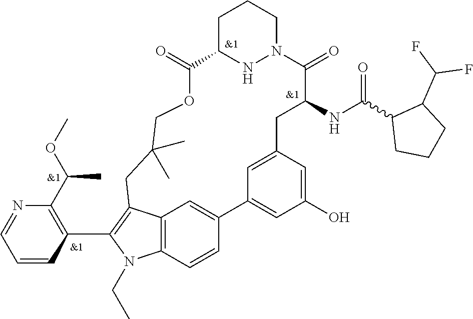 |
| AA255 | 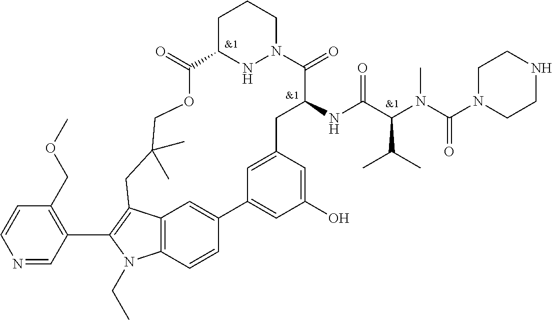 [SMILES: CCn1c(-c2cnccc2COC)c2c3cc(ccc31)-c1cc(O)cc(c1)C[C@H](NC(=O)[C@H](C(C)C)N(C)C(=O)N1CCNCC1)C(=O)N1CCC[C@H](N1)C(=O)OCC(C)(C)C2] |
| AA256 | 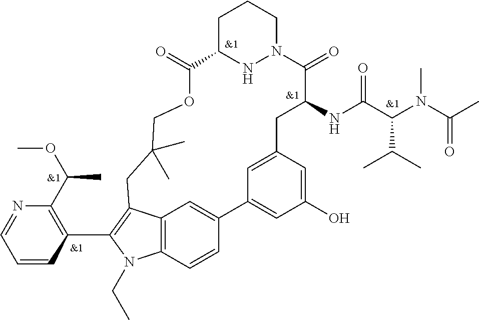 |
| AA257 | 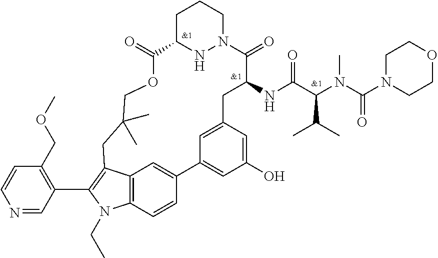 [SMILES: CCn1c(-c2cnccc2COC)c2c3cc(ccc31)-c1cc(O)cc(c1)C[C@H](NC(=O)[C@H](C(C)C)N(C)C(=O)N1CCOCC1)C(=O)N1CCC[C@H](N1)C(=O)OCC(C)(C)C2] |
| AA258 | 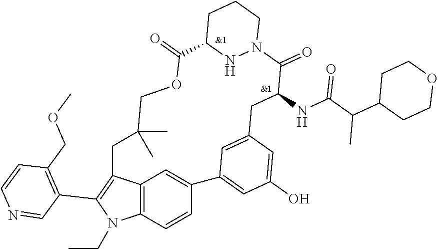 [SMILES: CCn1c(-c2cnccc2COC)c2c3cc(ccc31)-c1cc(O)cc(c1)C[C@H](NC(=O)C(C)C1CCOCC1)C(=O)N1CCC[C@H](N1)C(=O)OCC(C)(C)C2] |
| AA259 | 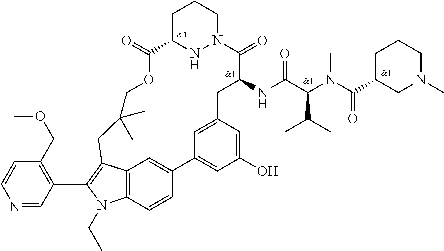 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCN(C)C1)C(=O)N1CCC[C@H](N1)C(=O)OCC(C)(C)C2] |
| AA260 | 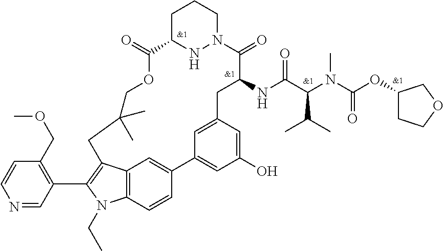 [SMILES: CCn1c(-c2cnccc2COC)c2c3cc(ccc31)-c1cc(O)cc(c1)C[C@H](NC(=O)[C@H](C(C)C)N(C)C(=O)O[C@H]1CCOC1)C(=O)N1CCC[C@H](N1)C(=O)OCC(C)(C)C2] |
| AA261 | 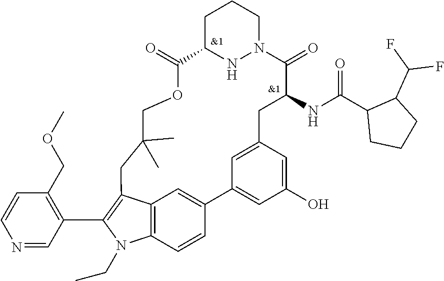 [SMILES: CCn1c(-c2cnccc2COC)c2c3cc(ccc31)-c1cc(O)cc(c1)C[C@H](NC(=O)C1CCCC1C(F)F)C(=O)N1CCC[C@H](N1)C(=O)OCC(C)(C)C2] |
| AA262 | 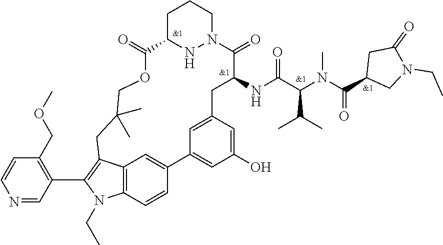 [SMILES: CCN1C[C@@H](C(=O)N(C)[C@H](C(=O)N[C@H]2Cc3cc(O)cc(c3)-c3ccc4c(c3)c(c(-c3cnccc3COC)n4CC)CC(C)(C)COC(=O)[C@@H]3CCCN(N3)C2=O)C(C)C)CC1=O] |
| AA263 | 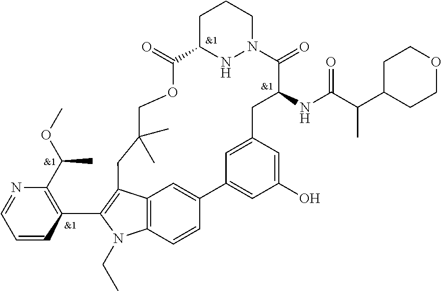 |
| AA264 | 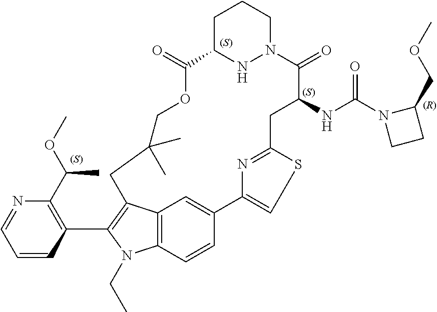 |
| AA265 | 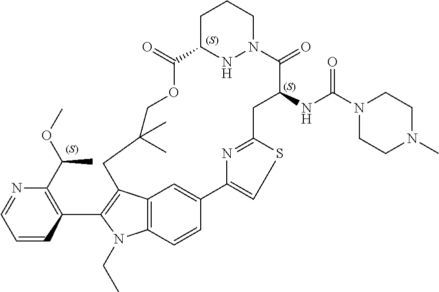 |
| AA266 | 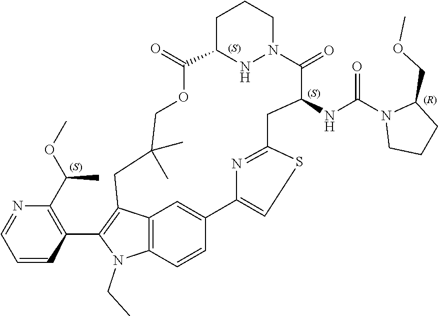 |
| AA267 | 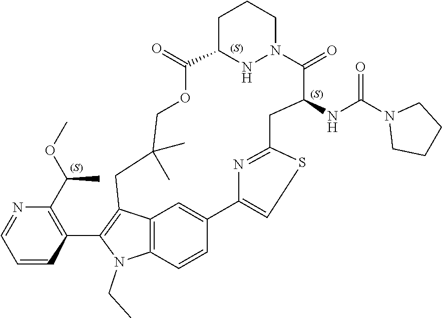 |
| AA268 | 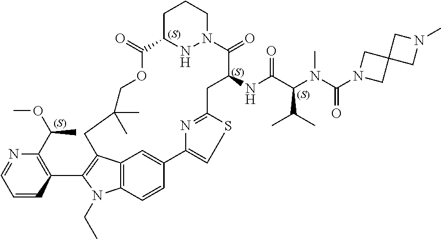 |
| AA270 | 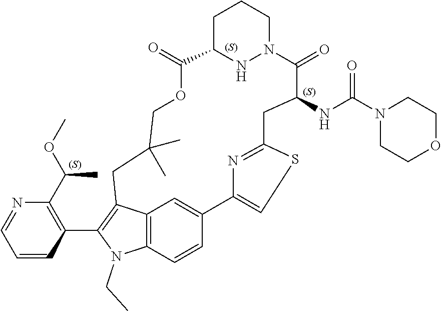 |
| AA271 | 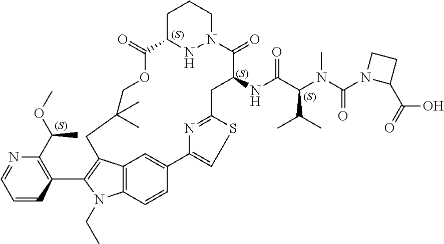 |
| AA272 | 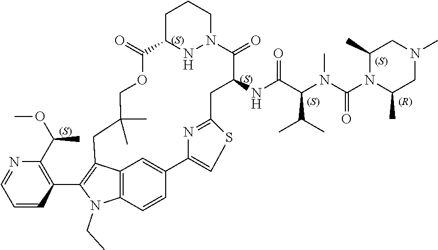 |
| AA273 | 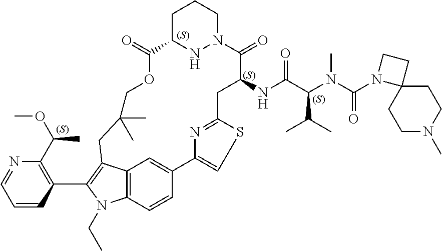 |
| AA274 | 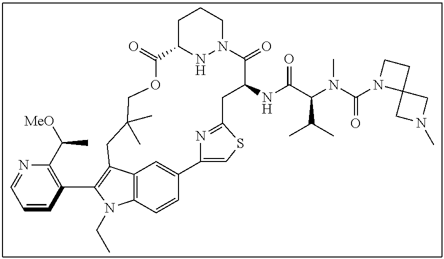 |
| AA275 | 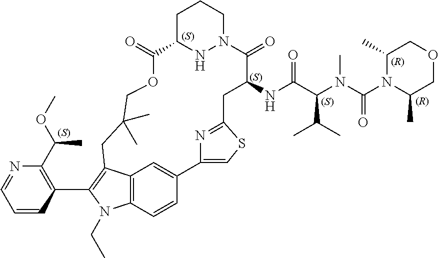 |
| AA276 | 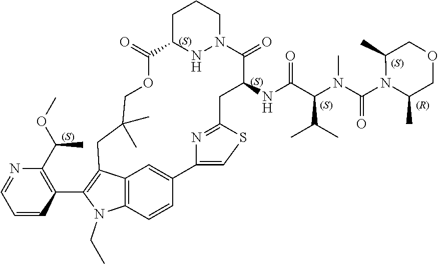 |
| AA277 | 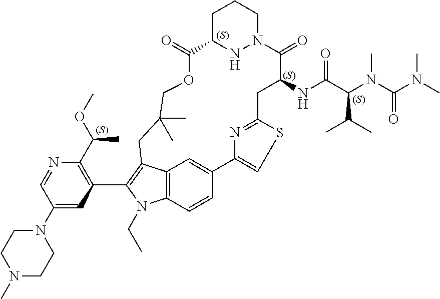 |
| AA278 | 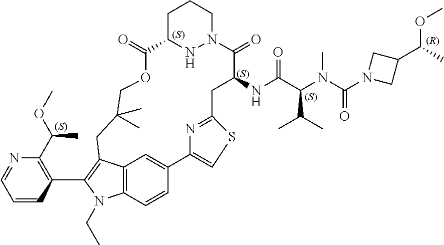 |
| AA279 | 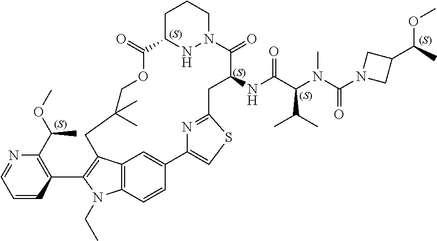 |
| AA280 | 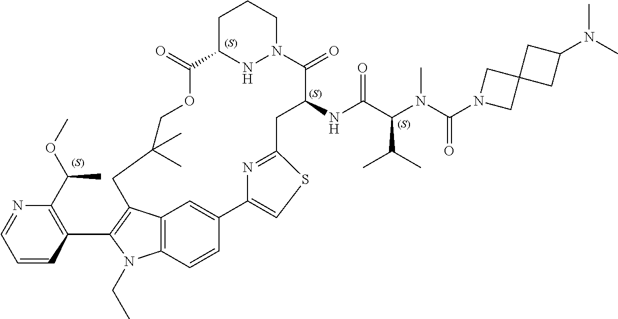 |
| AA281 | 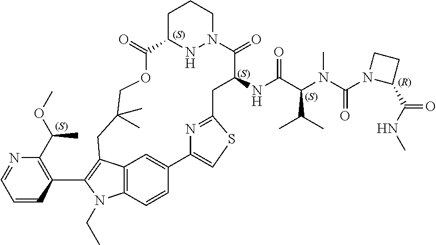 |
| AA282 | 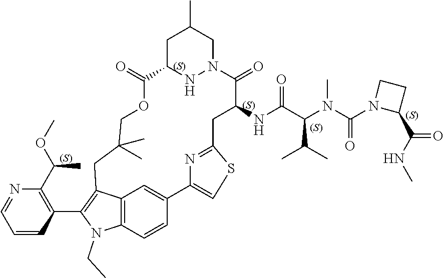 |
| AA283 | 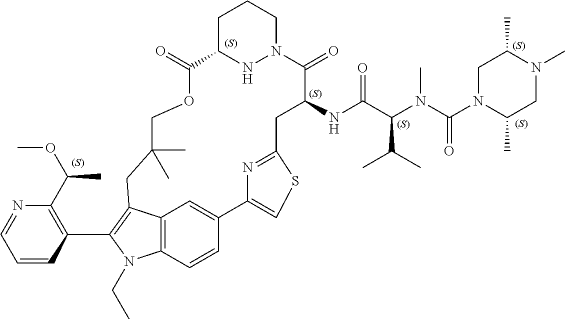 |
| AA284 | 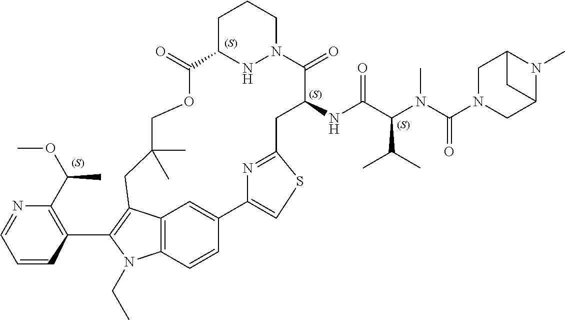 |
| AA285 | 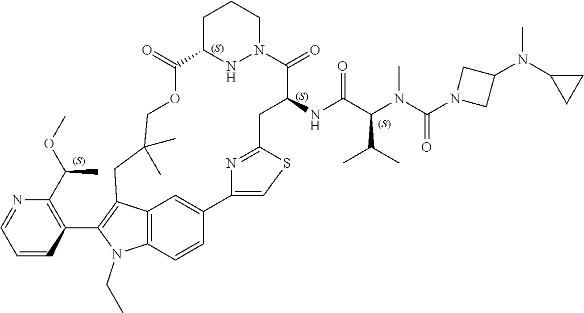 |
| AA286 | 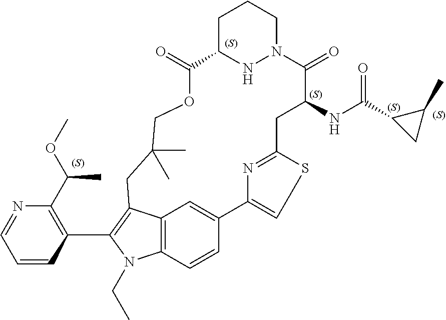 |
| AA287 | 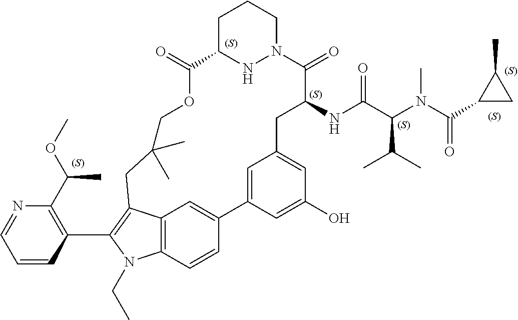 |
| AA288 | 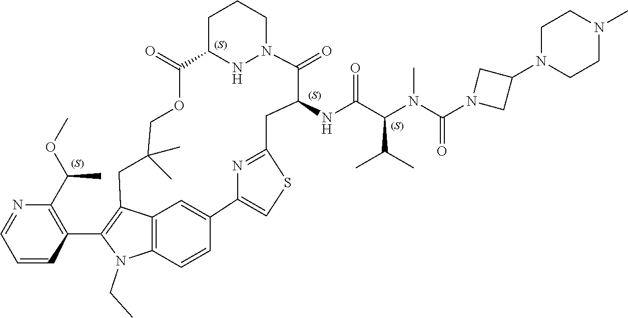 |
| AA289 | 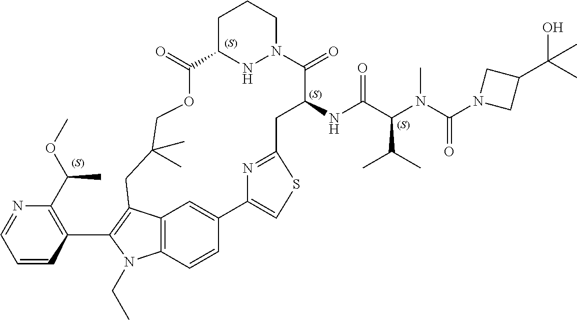 |
| AA290 | 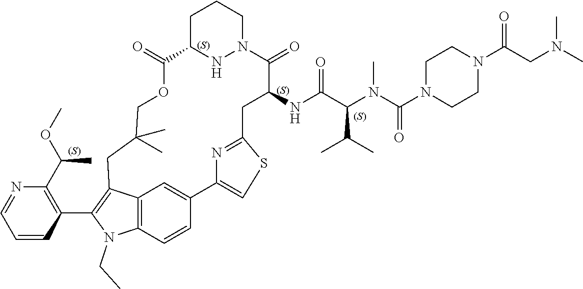 |
| AA291 | 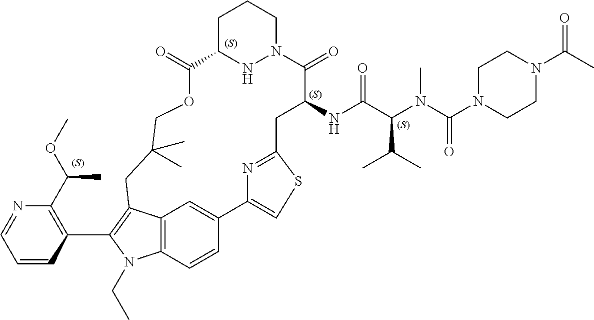 |
| AA292 | 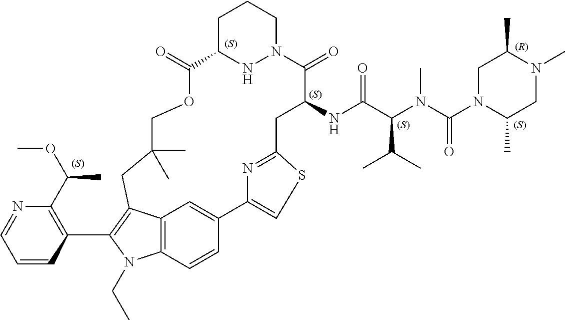 |
| AA293 | 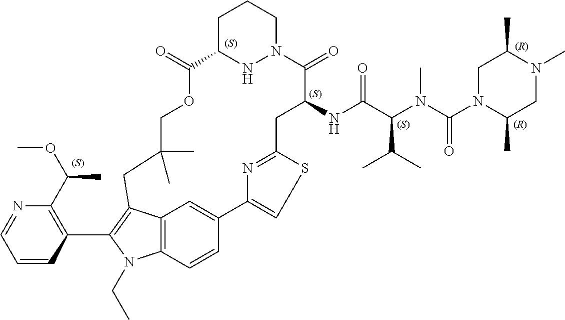 |
| AA294 | 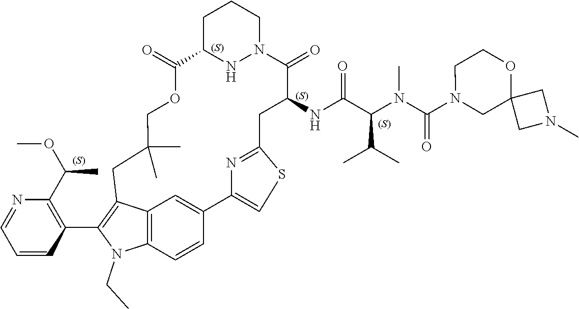 |
| AA295 | 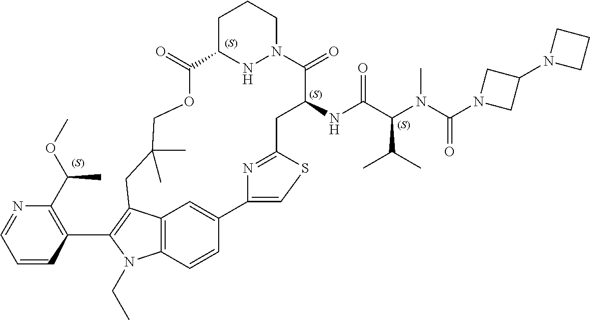 |
| AA296 | 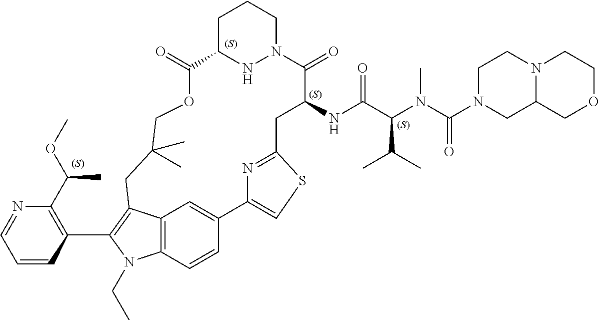 |
| AA297 | 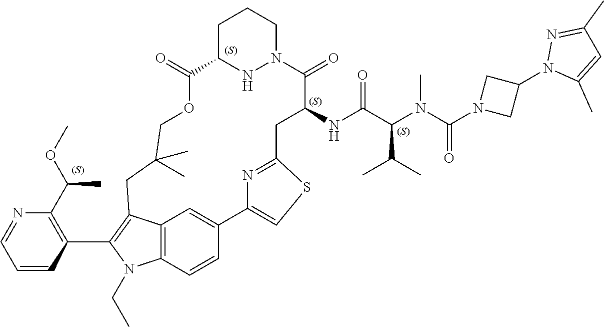 |
| AA298 | 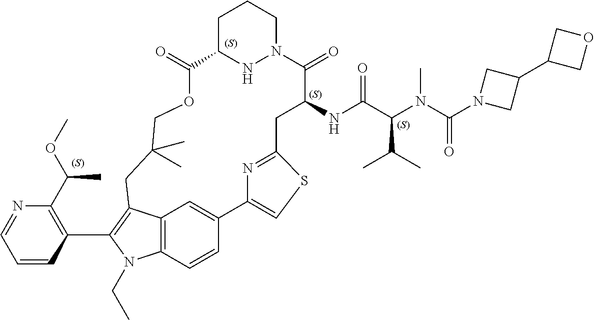 |
| AA299 | 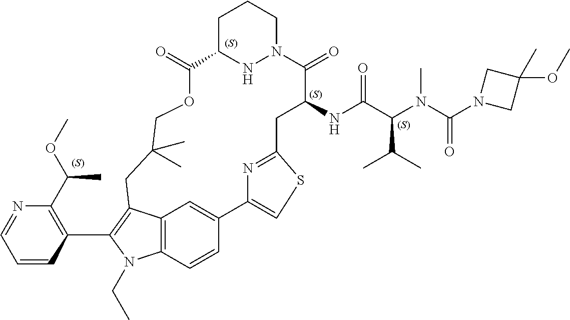 |
| AA300 | 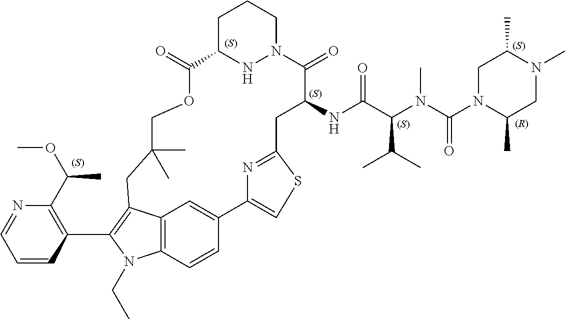 |
| AA301 | 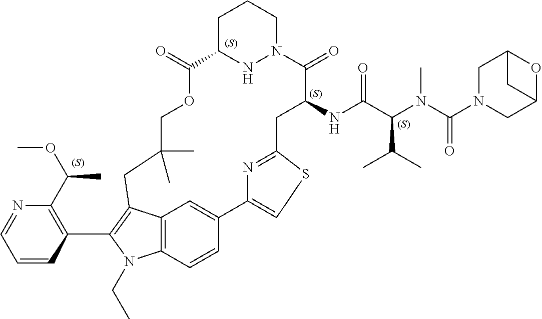 |
| AA302 | 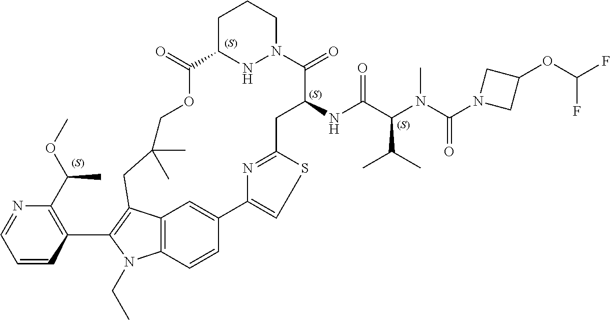 |
| AA303 | 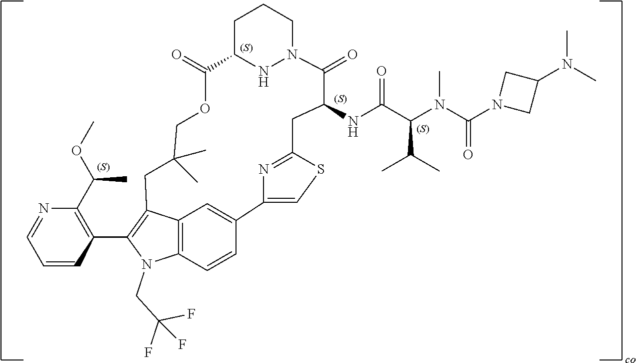 |
| AA304 | 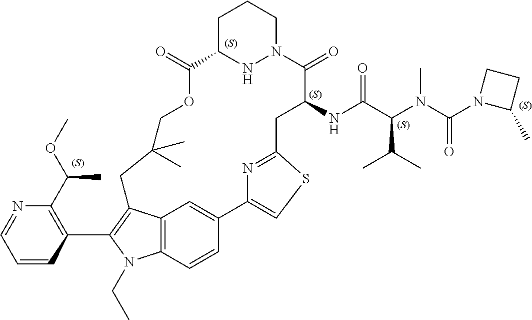 |
| AA305 | 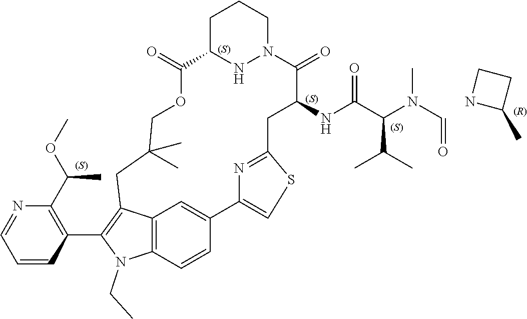 |
| AA306 | 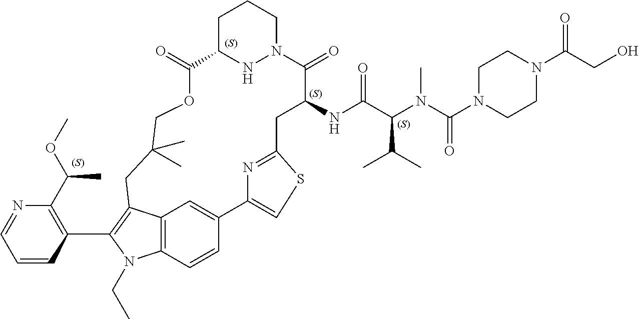 |
| AA307 | 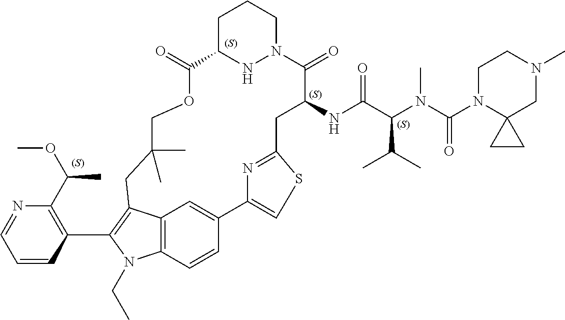 |
| AA308 | 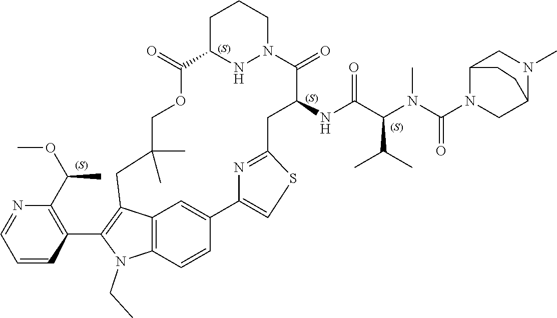 |
| AA309 | 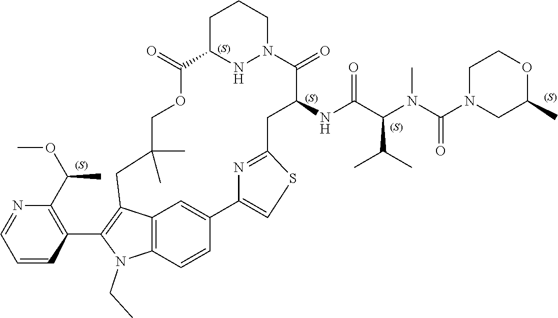 |
| AA310 | 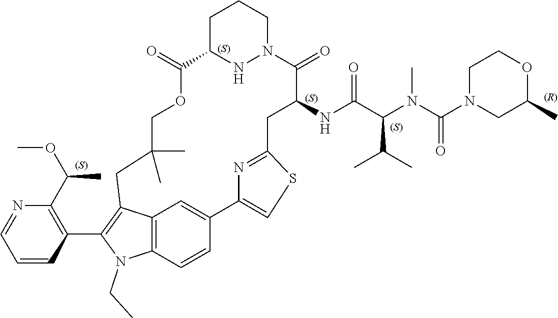 |
| AA311 | 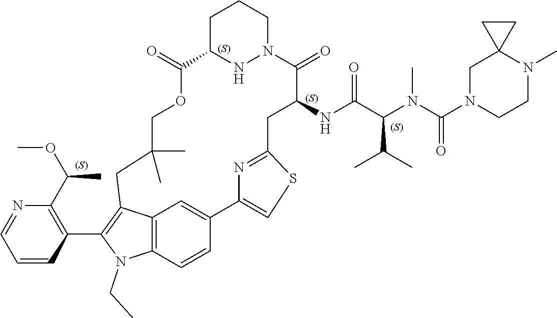 |
| AA312 | 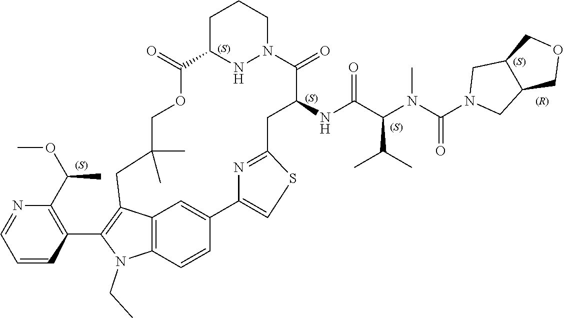 |
| AA313 | 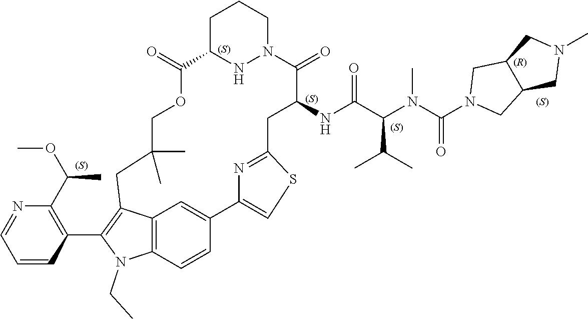 |
| AA314 | 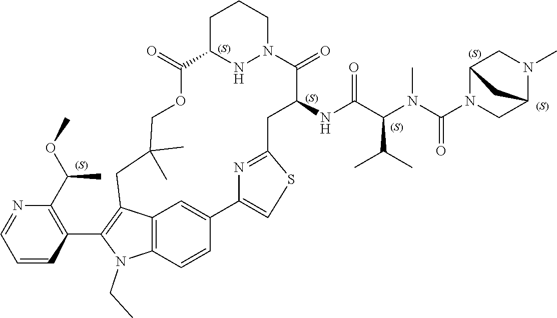 |
| AA315 | 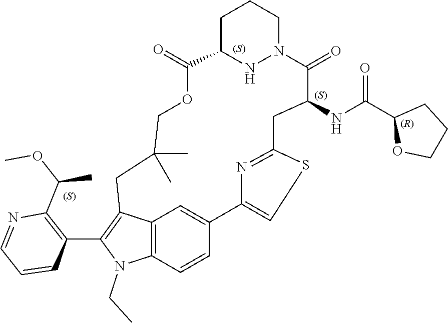 |
| AA316 | 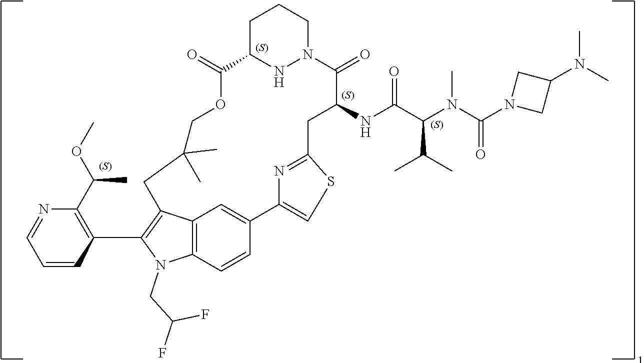 |
| AA317 | 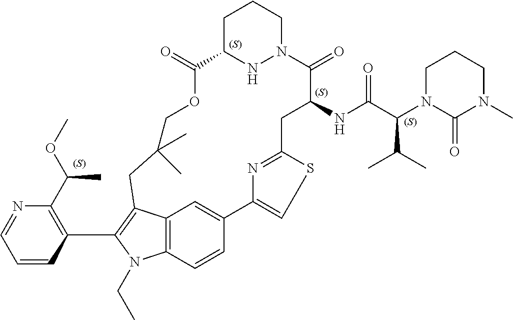 |
| AA318 | 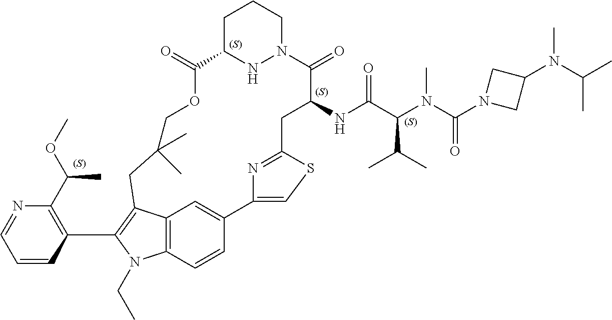 |
| AA319 | 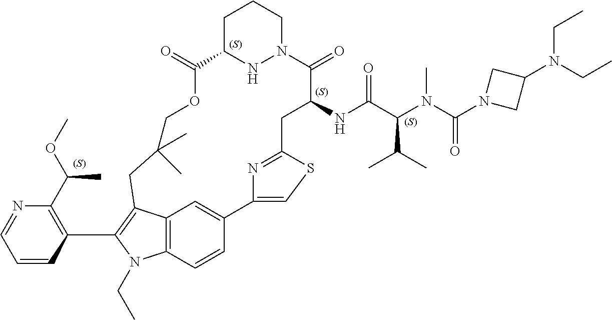 |
| AA320 | 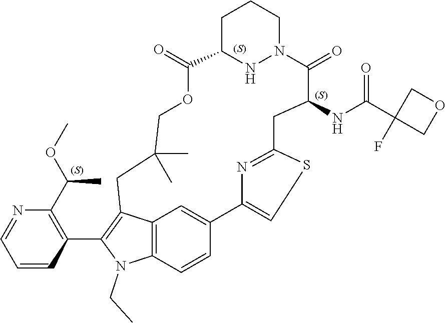 |
| AA321 | 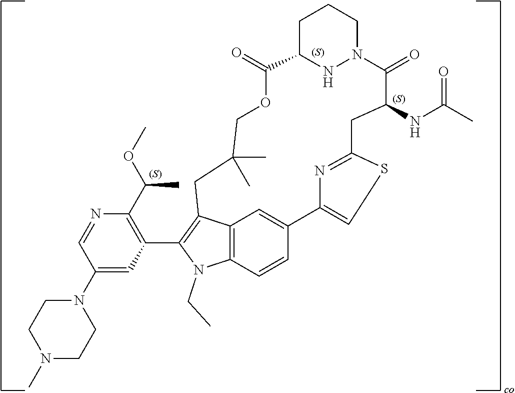 |
| AA322 | 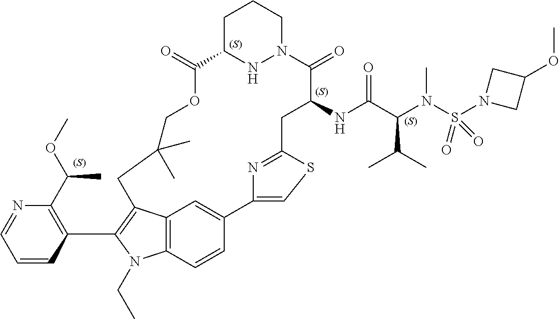 |
| AA323 | 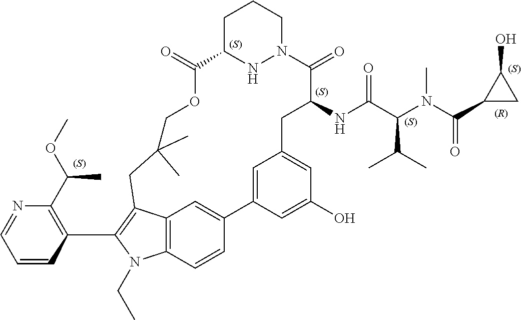 |
| AA324 | 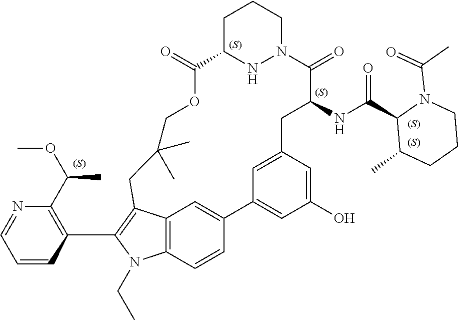 |
| AA325 | 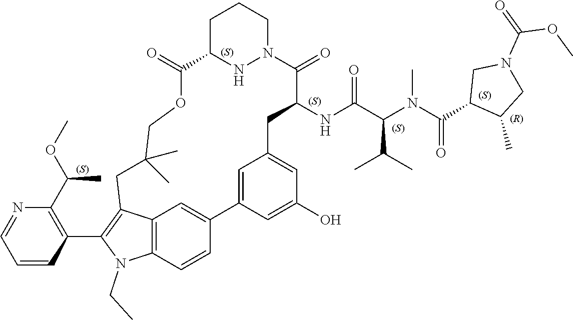 |
| AA326 | 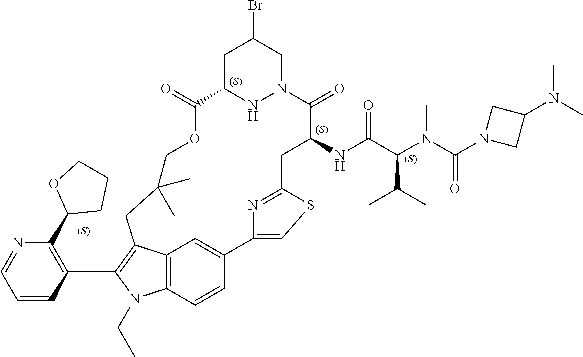 |
| AA327 | 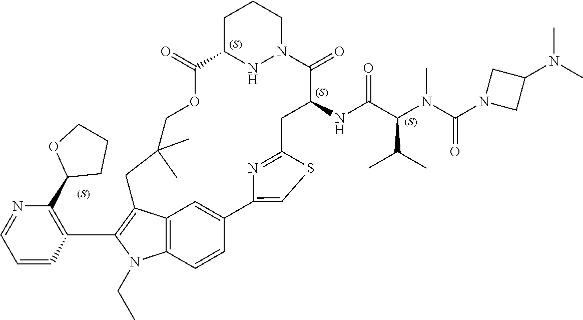 |
| AA328 | 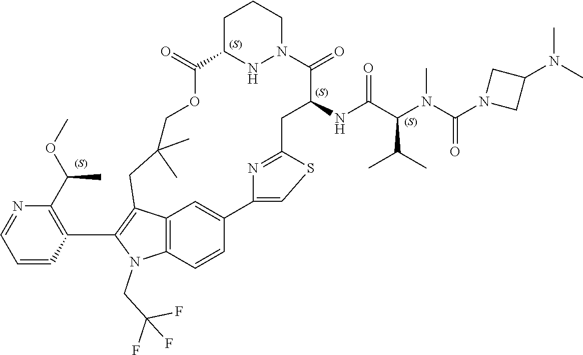 |
| AA329 | 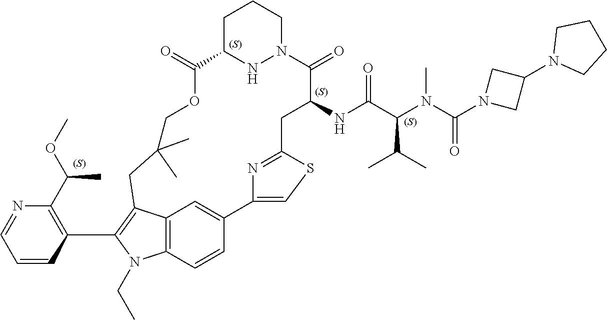 |
| AA330 | 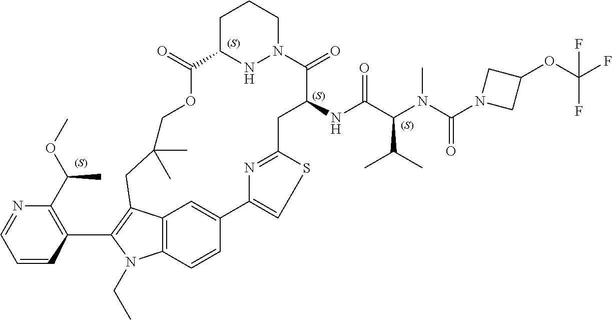 |
| AA331 | 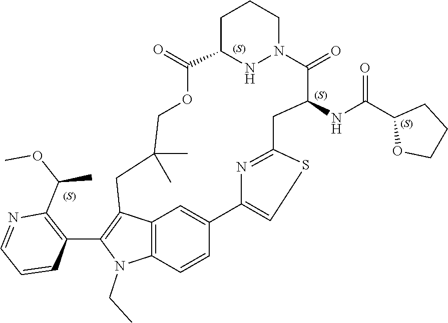 |
| AA332 | 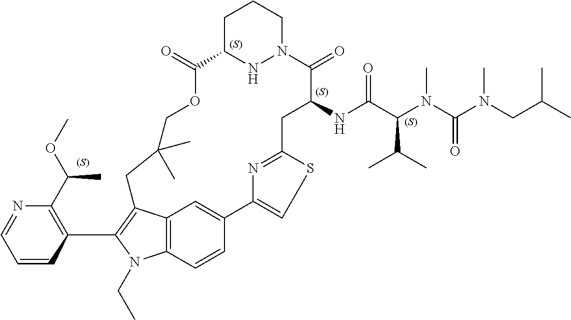 |
| AA333 | 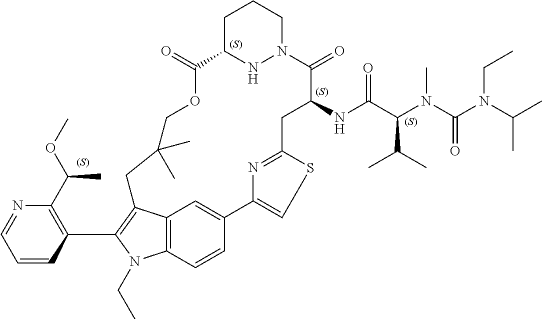 |
| AA334 | 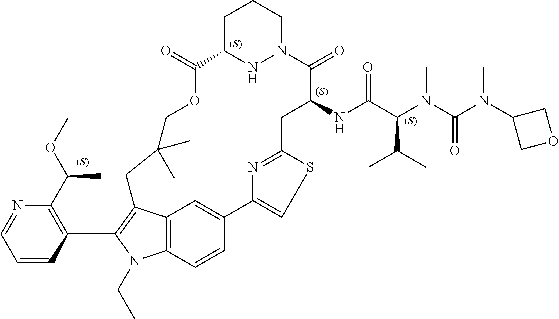 |
| AA335 | 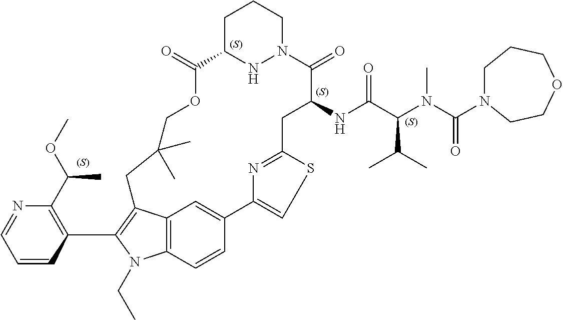 |
| AA336 | 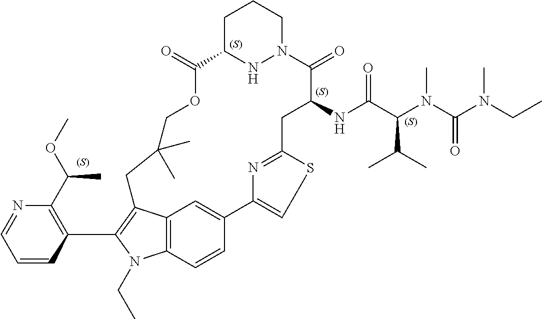 |
| AA337 | 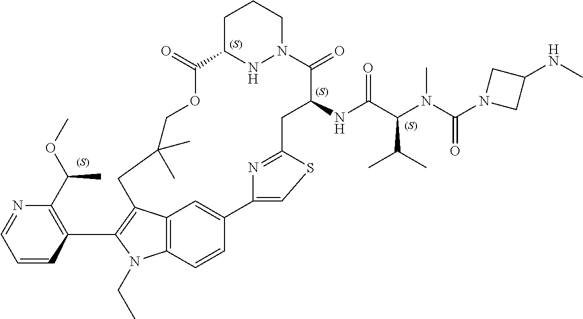 |
| AA338 | 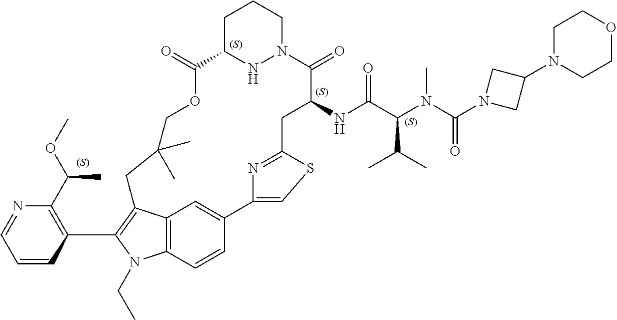 |
| AA339 | 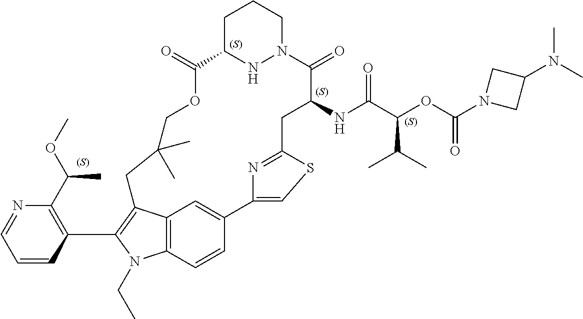 |
| AA340 | 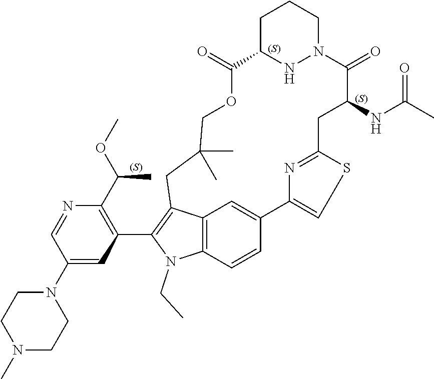 |
| AA341 | 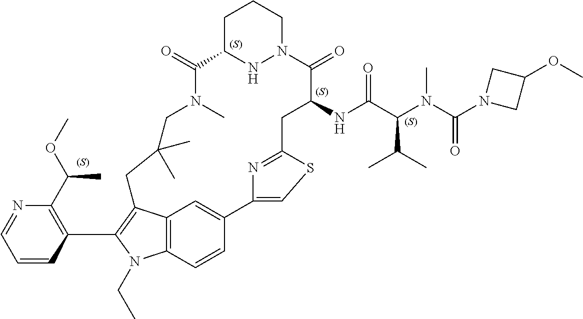 |
| AA342 | 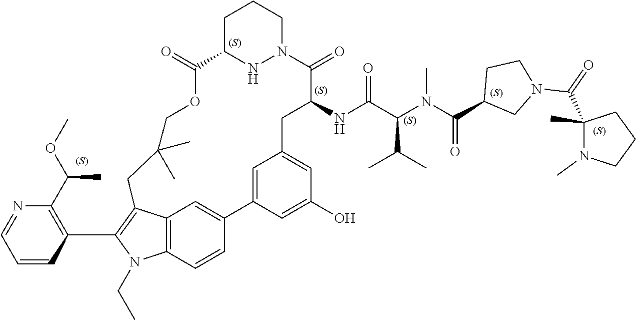 |
| AA343 | 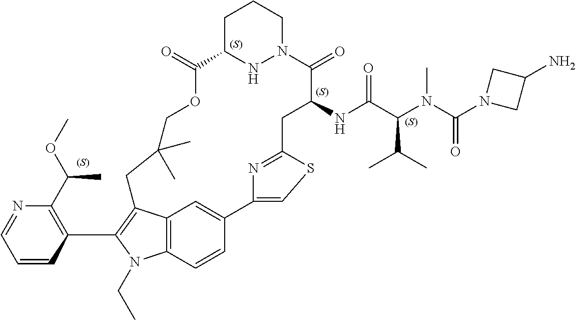 |
| AA344 | 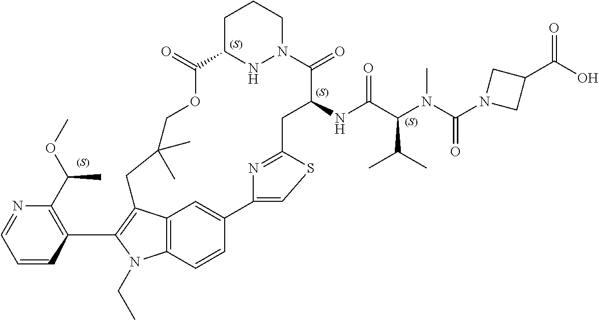 |
| AA345 | 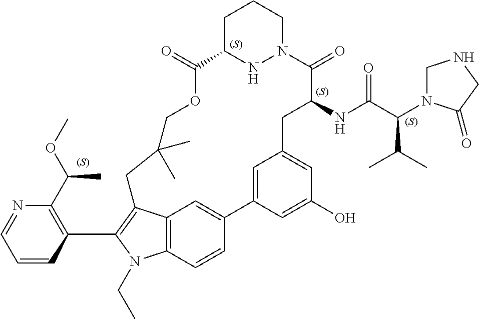 |
| AA346 | 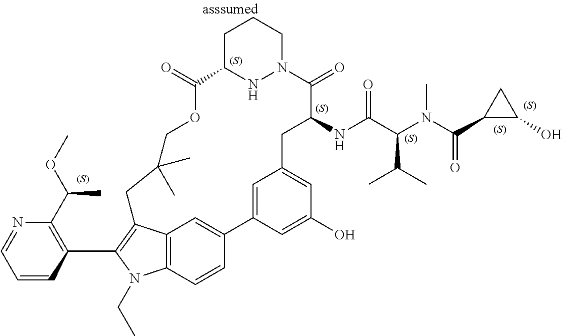 |
| AA347 | 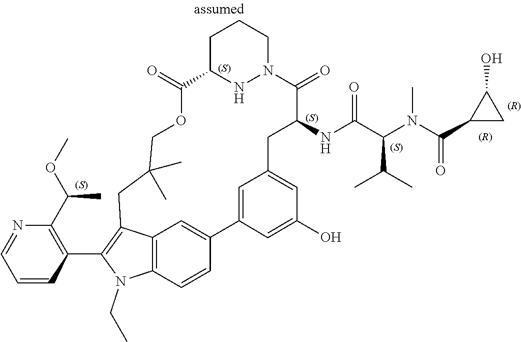 |
| AA348 | 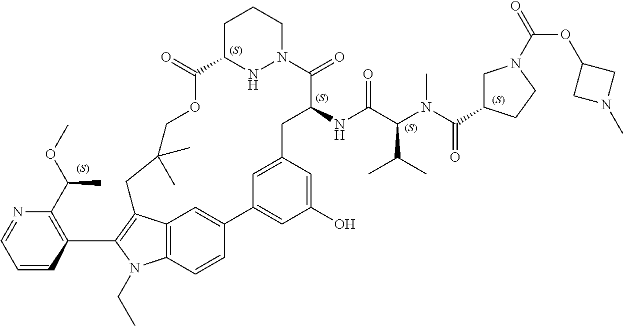 |
| AA349 | 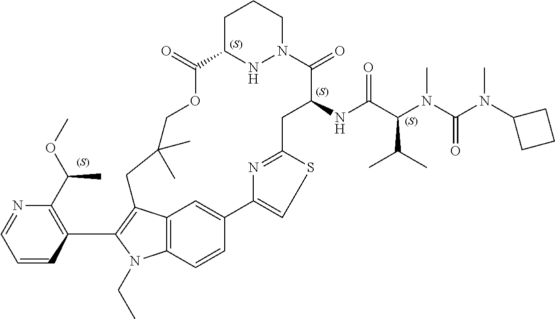 |
| AA350 | 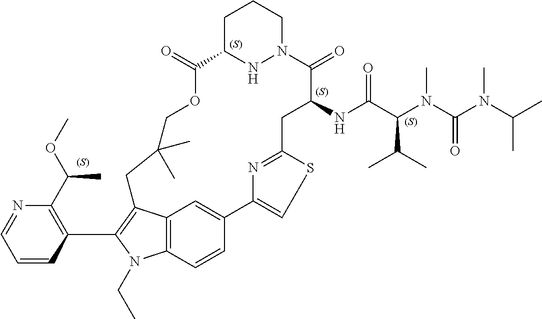 |
| AA351 | 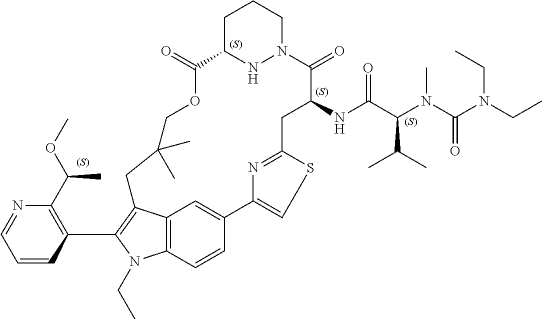 |
| AA352 | 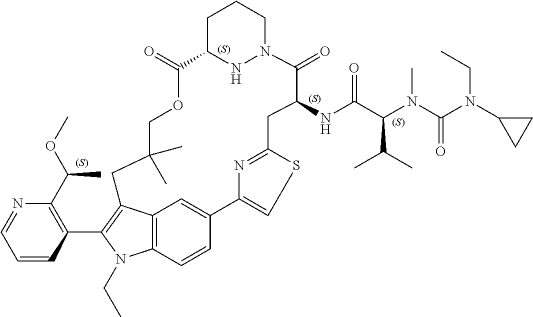 |
| AA353 | 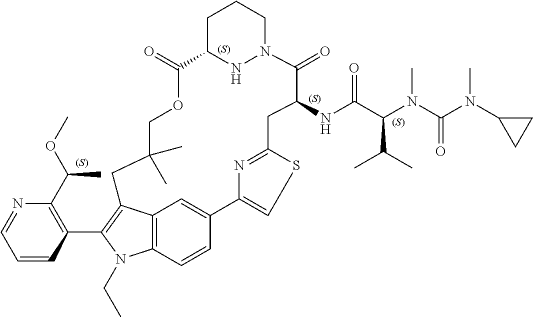 |
| AA354 | 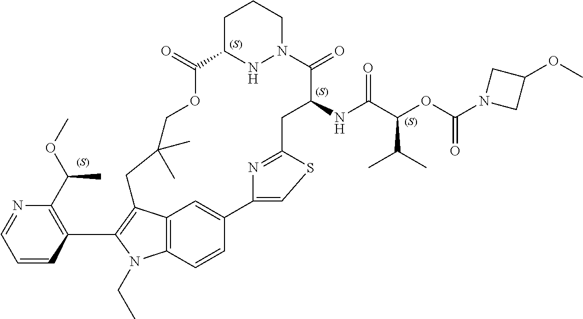 |
| AA355 | 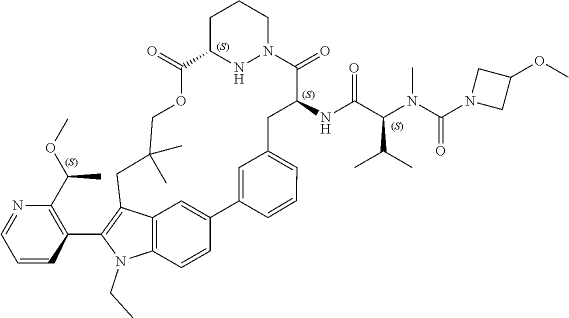 |
| AA356 | 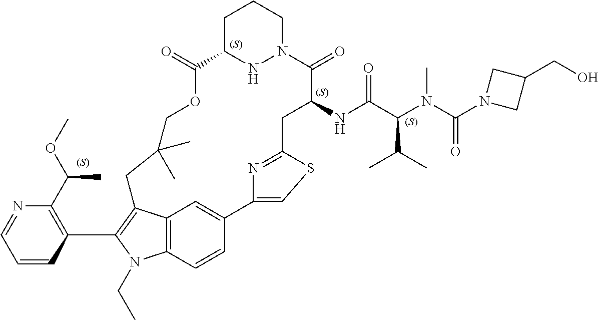 |
| AA357 | 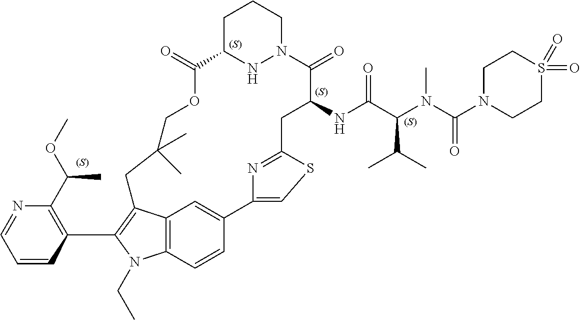 |
| AA358 | 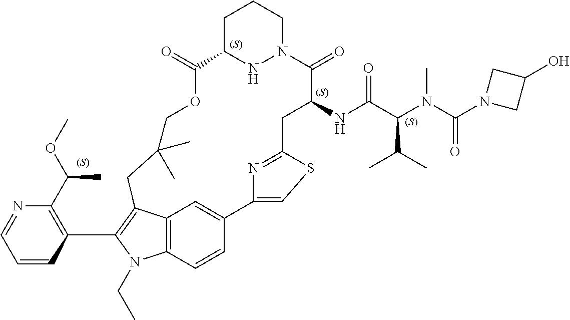 |
| AA359 | 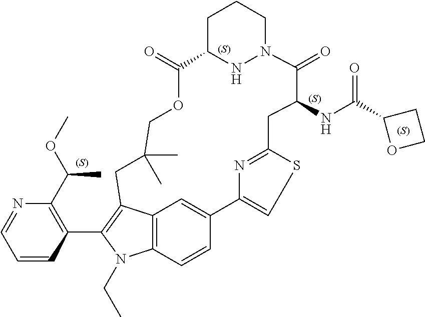 |
| AA360 | 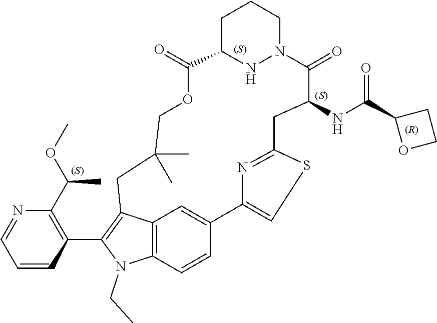 |
| AA361 | 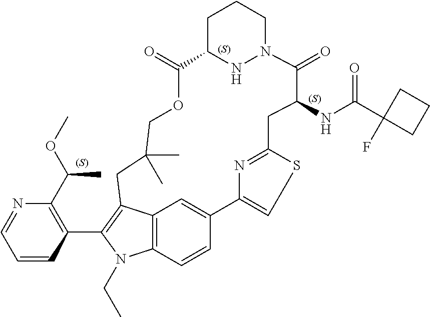 |
| AA362 | 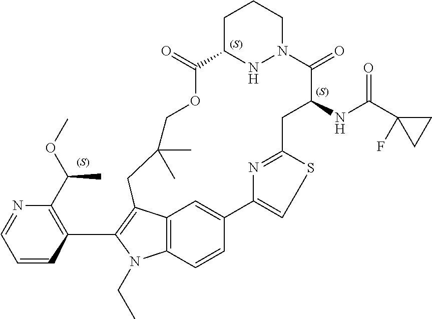 |
| AA363 | 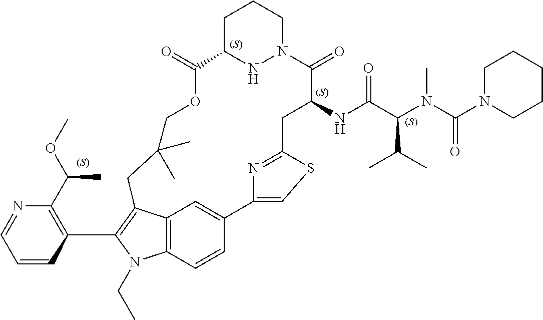 |
| AA364 | 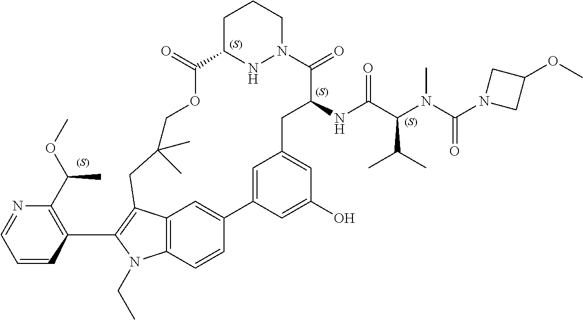 |
| AA365 | 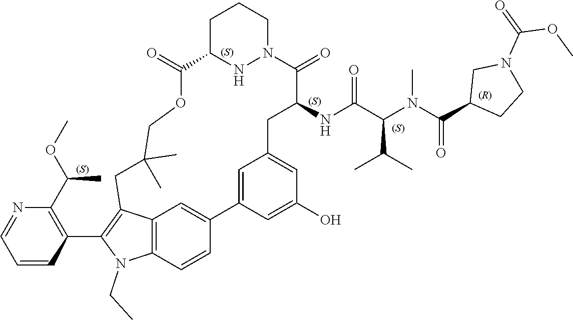 |
| AA366 | 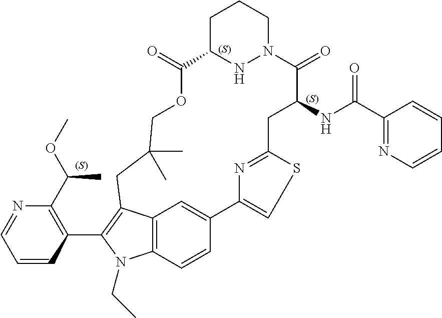 |
| AA367 | 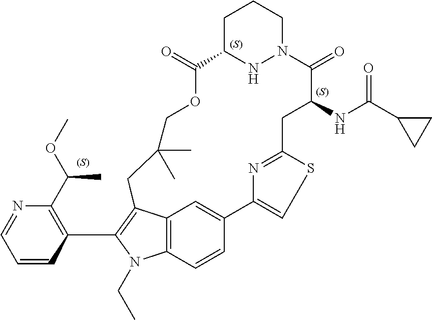 |
| AA368 | 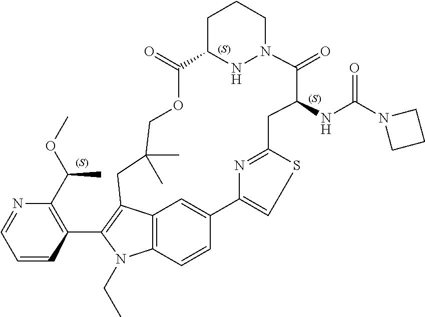 |
| AA369 | 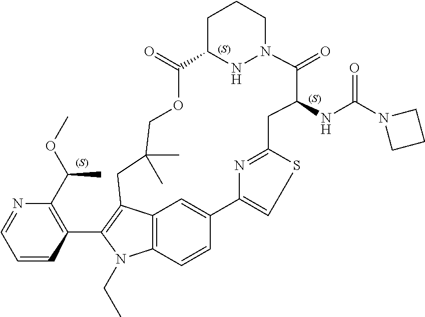 |
| AA370 | 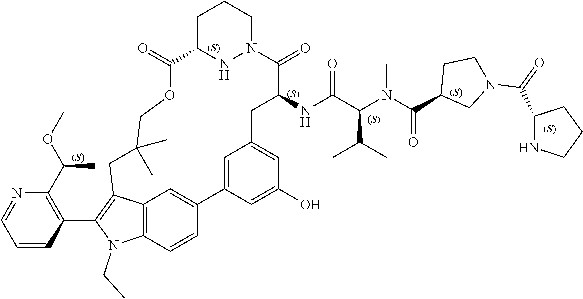 |
| AA371 | 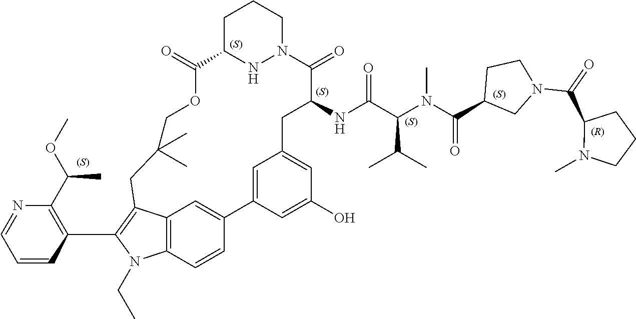 |
| AA372 | 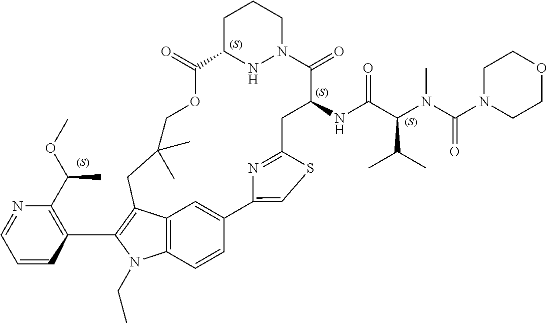 |
| AA373 | 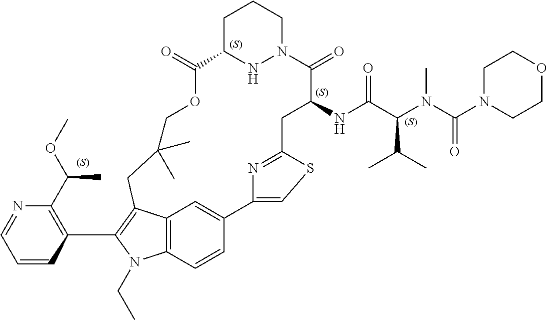 |
| AA374 | 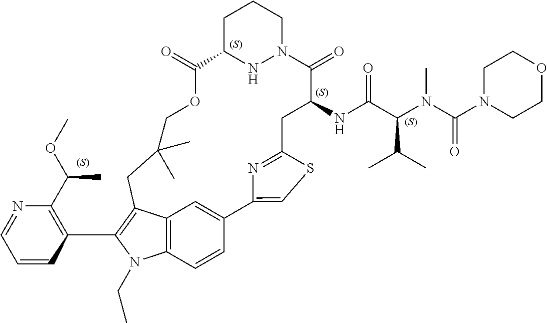 |
| AA375 | 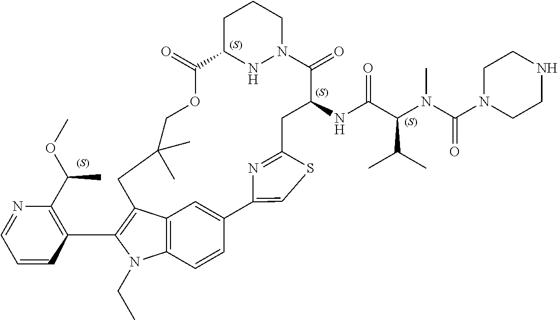 |
| AA376 | 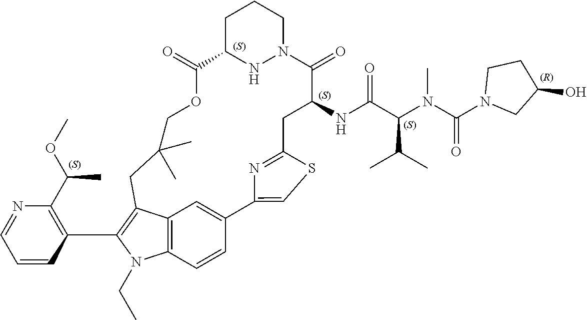 |
| AA377 | 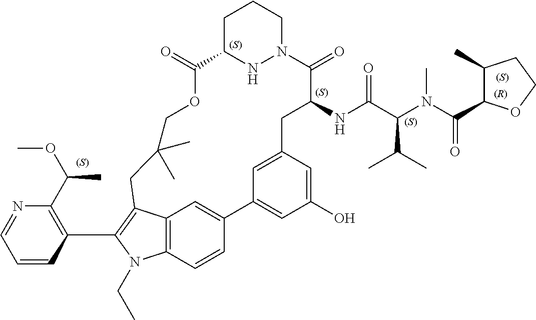 |
| AA378 | 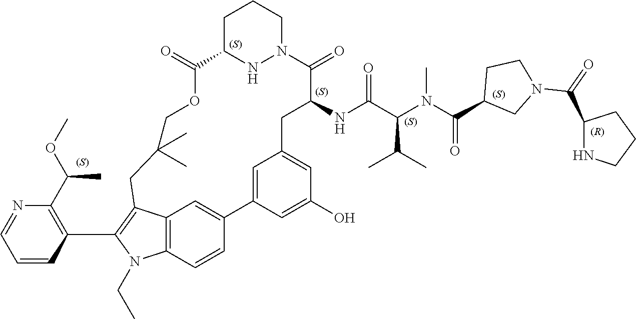 |
| AA379 | 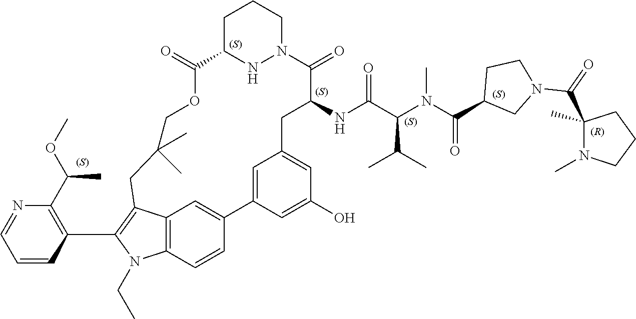 |
| AA380 | 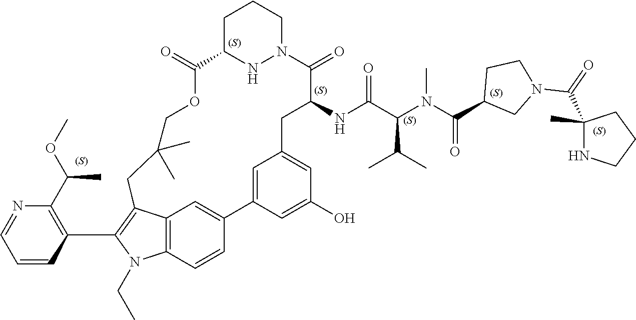 |
| AA381 | 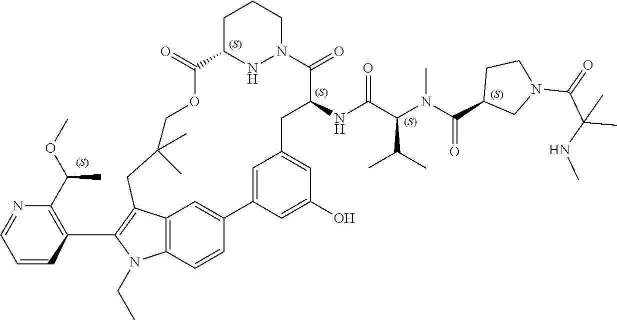 |
| AA382 | 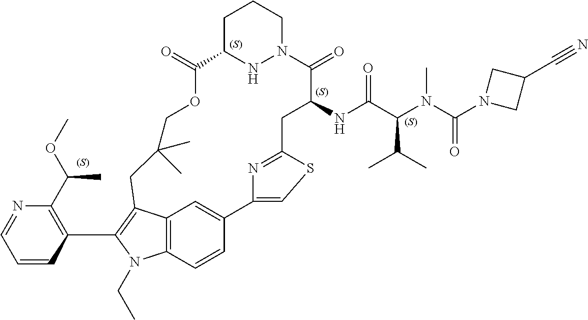 |
| AA383 | 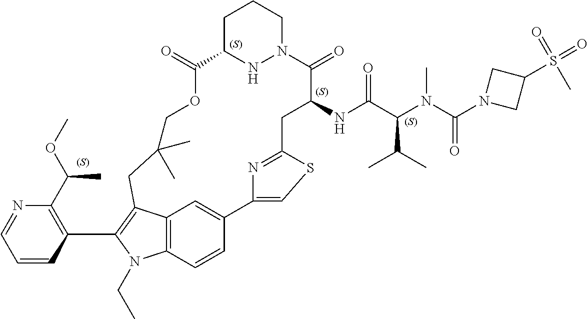 |
| AA384 | 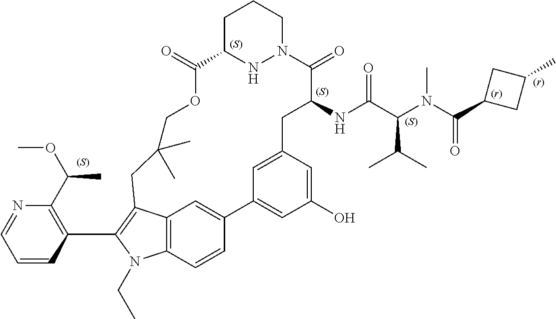 |
| AA385 | 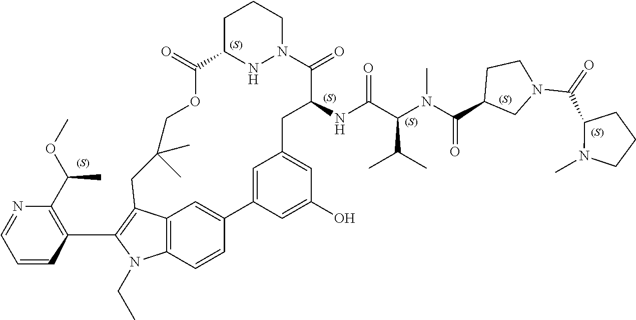 |
| AA386 | 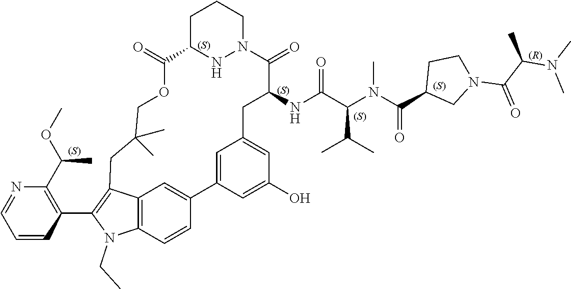 |
| AA387 | 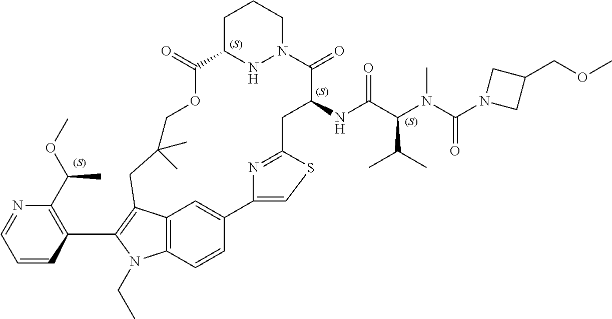 |
| AA388 | 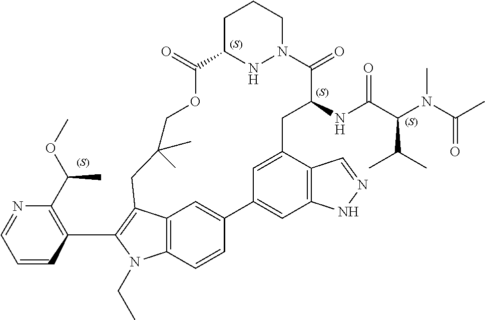 |
| AA389 | 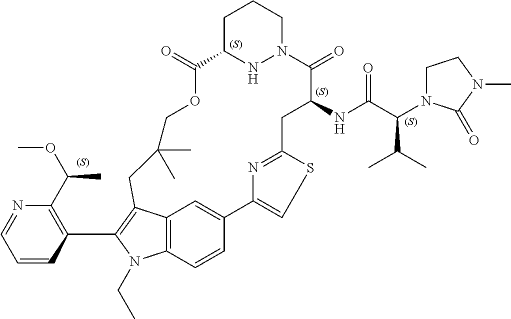 |
| AA391 | 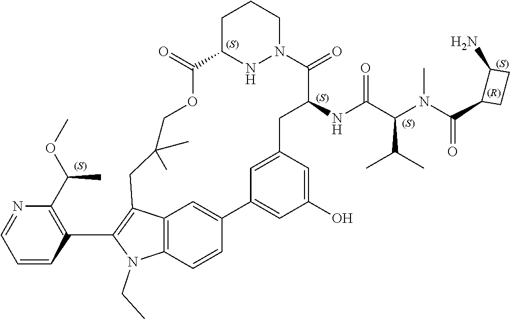 |
| AA392 | 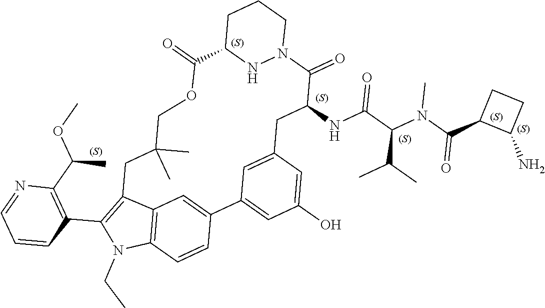 |
| AA393 | 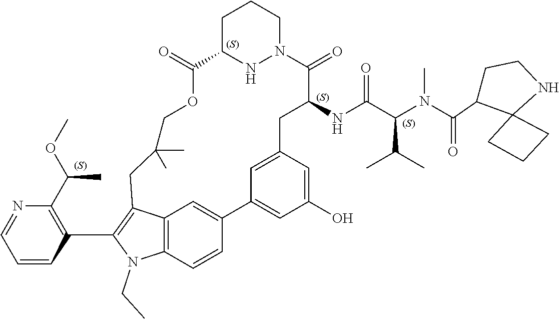 |
| AA394 | 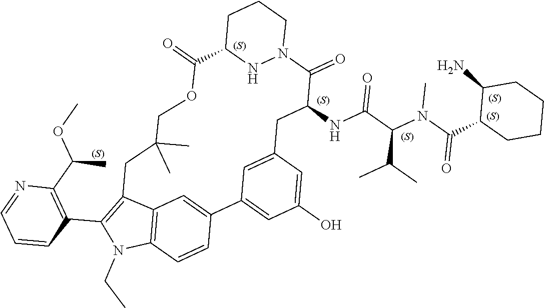 |
| AA395 | 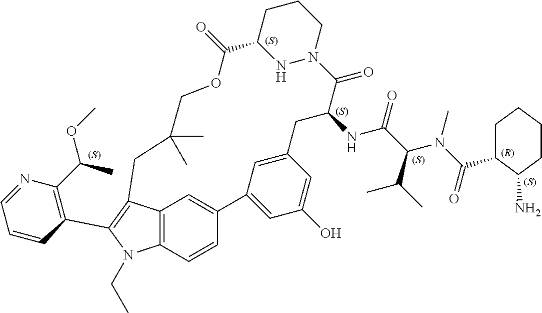 |
| AA396 | 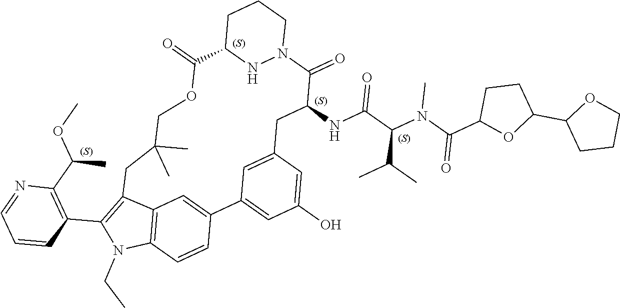 |
| AA397 | 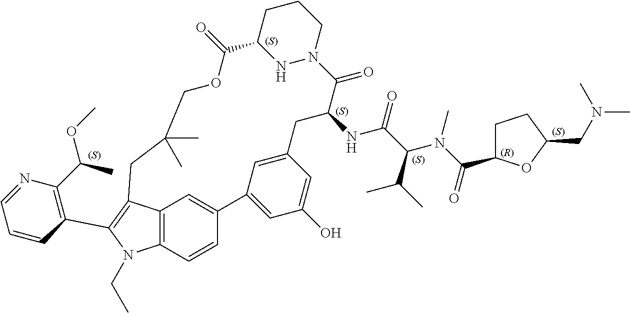 |
| AA398 | 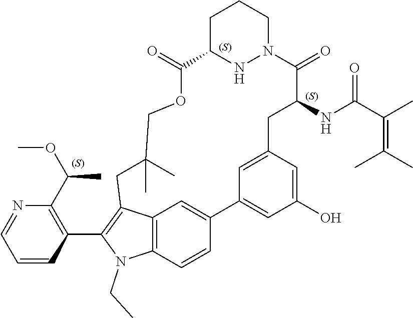 |
| AA399 | 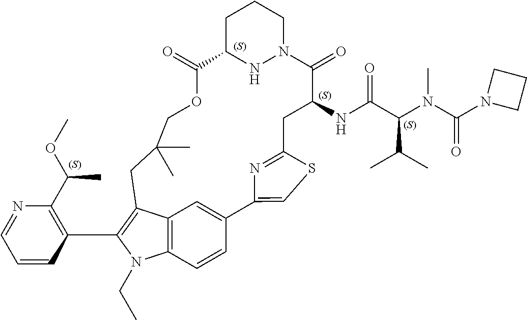 |
| AA400 | 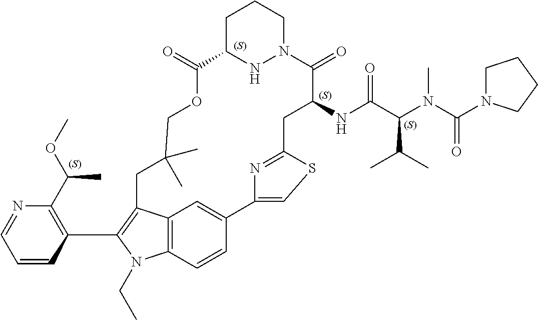 |
| AA401 | 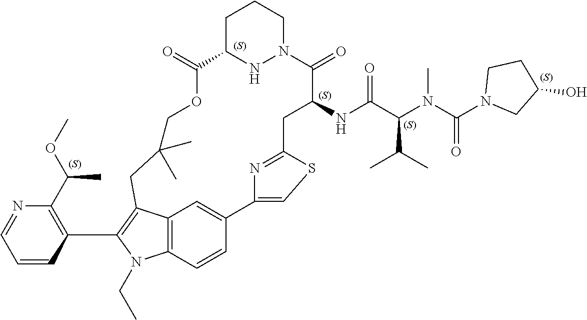 |
| AA402 | 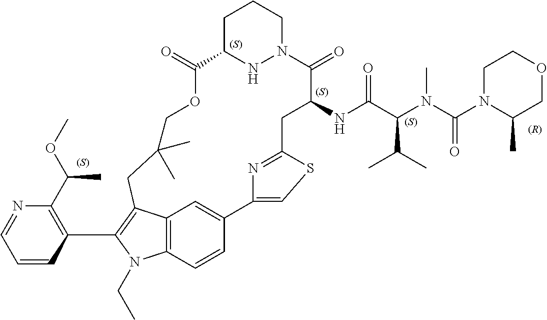 |
| AA403 | 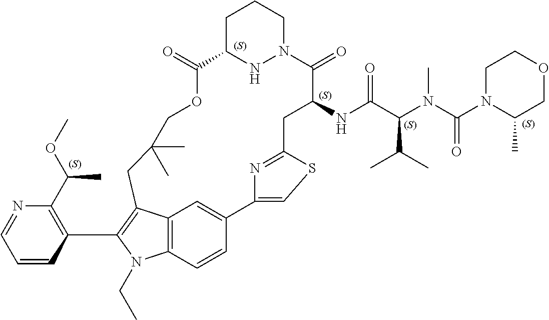 |
| AA404 | 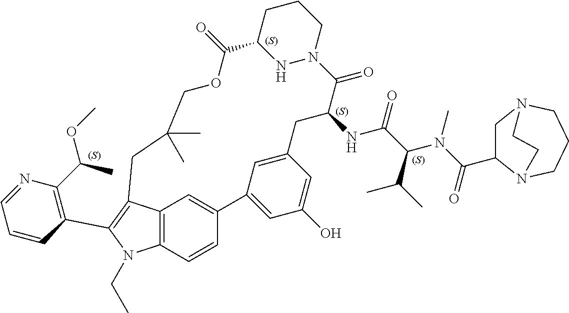 |
| AA405 | 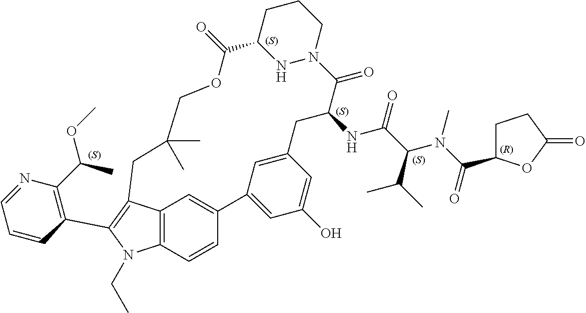 |
| AA406 | 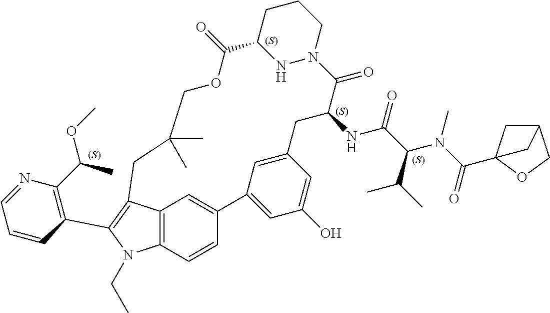 |
| AA407 | 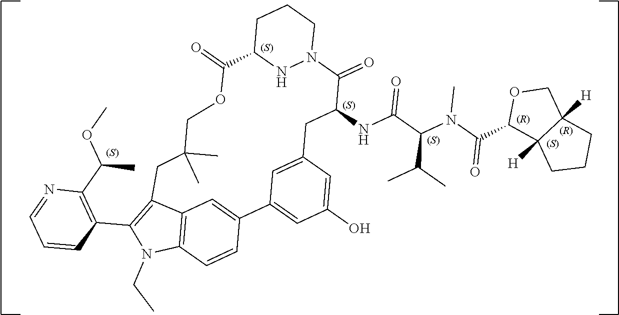 |
| AA408 | 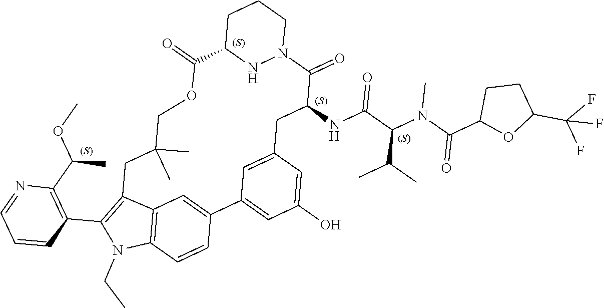 |
| AA409 | 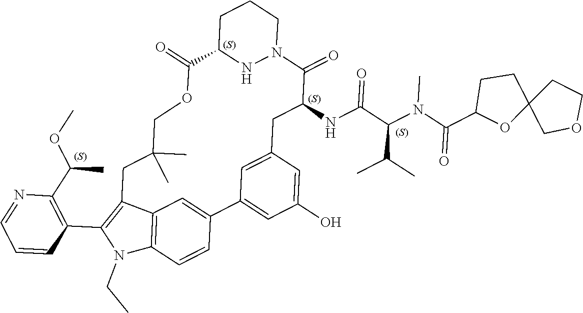 |
| AA410 | 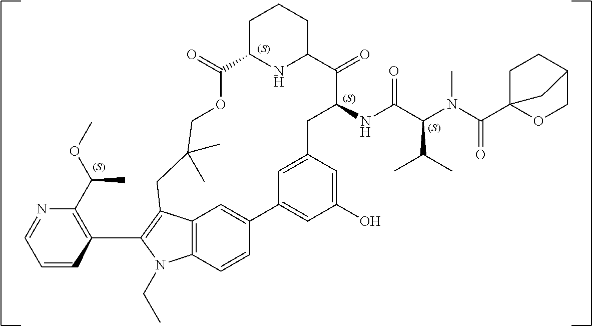 |
| AA411 | 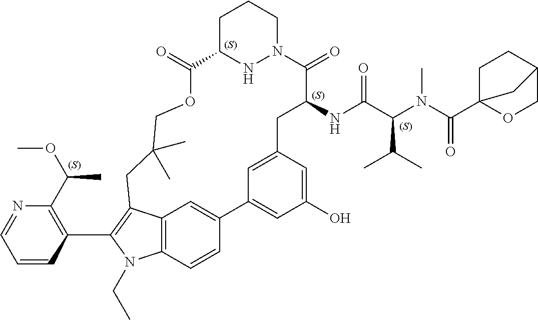 |
| AA412 | 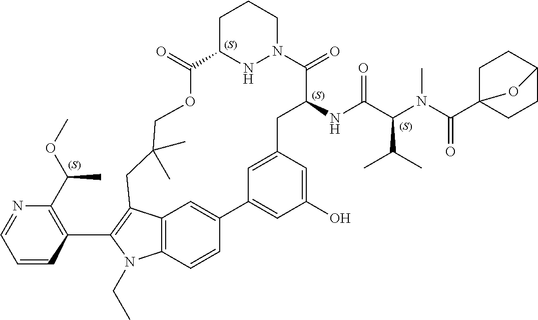 |
| AA413 | 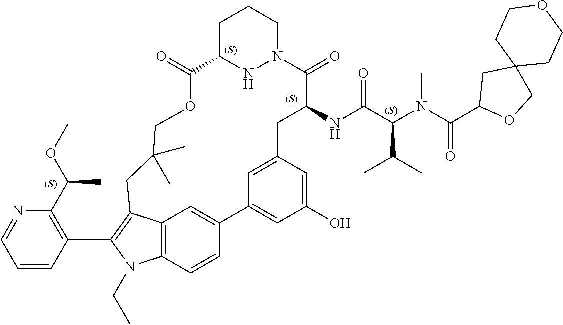 |
| AA414 | 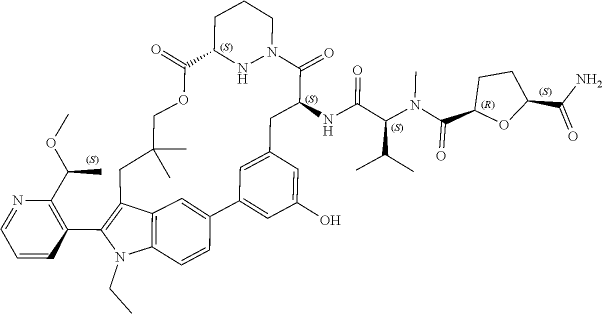 |
| AA415 | 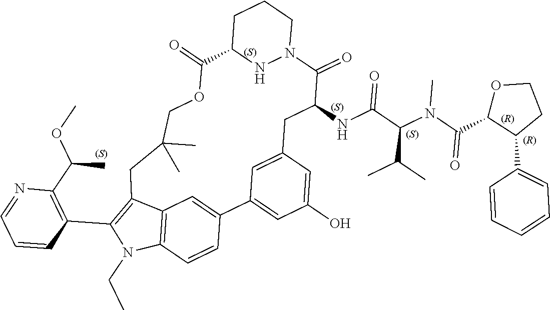 |
| AA416 | 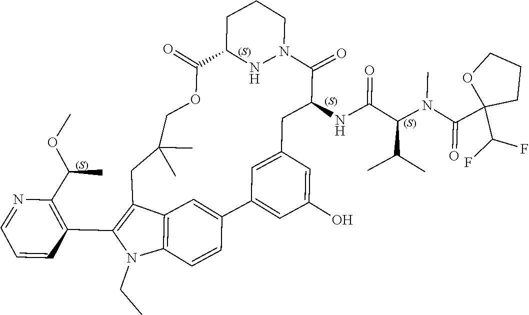 |
| AA417 | 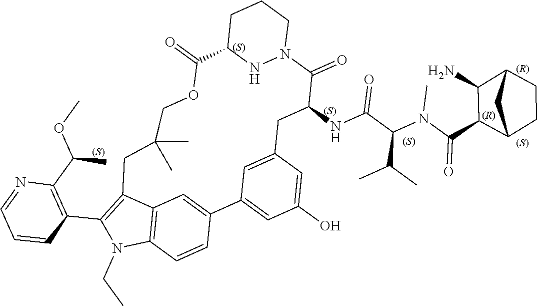 |
| AA418 | 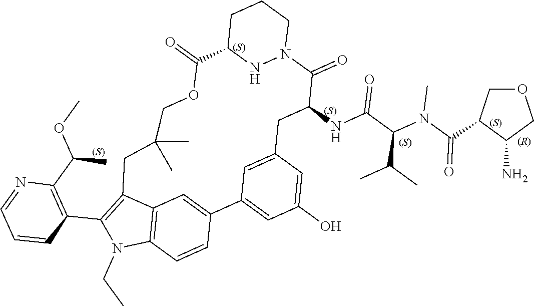 |
| AA419 | 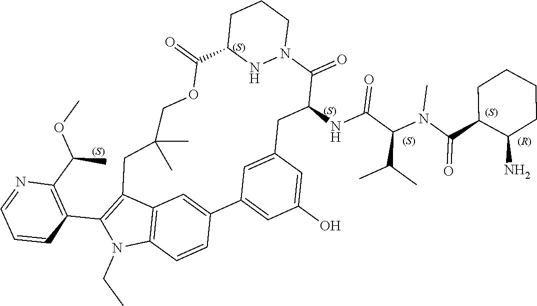 |
| AA420 | 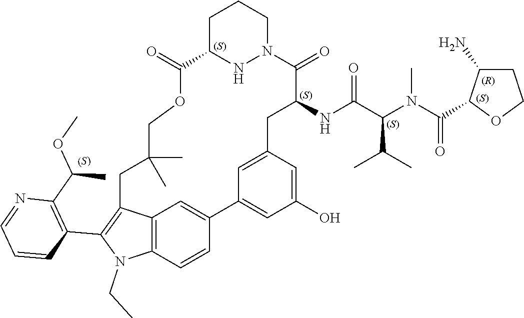 |
| AA421 | 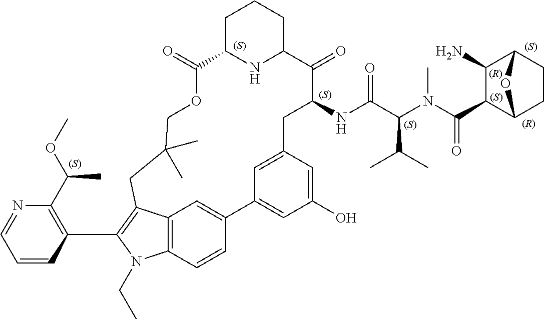 |
| AA422 | 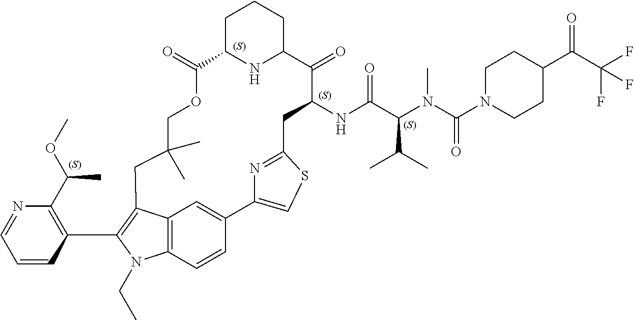 |
| AA423 | 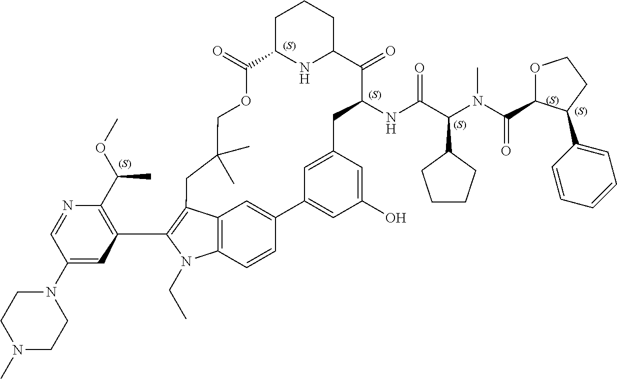 |
| AA424 | 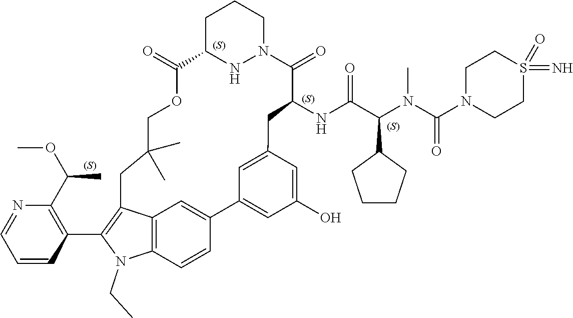 |
| AA425 | 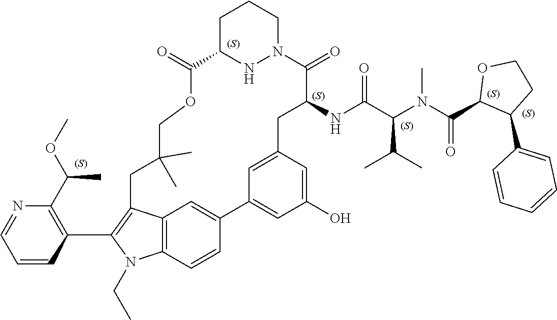 |
| AA426 | 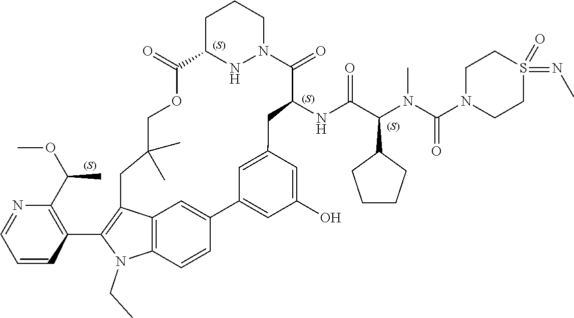 |
| AA427 | 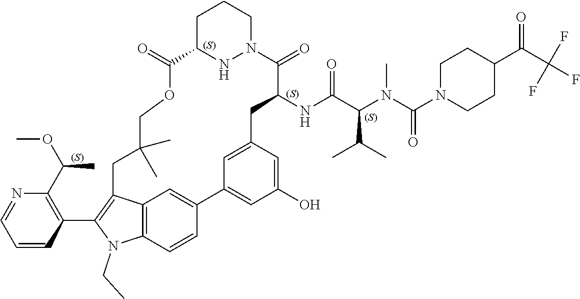 |
| AA428 | 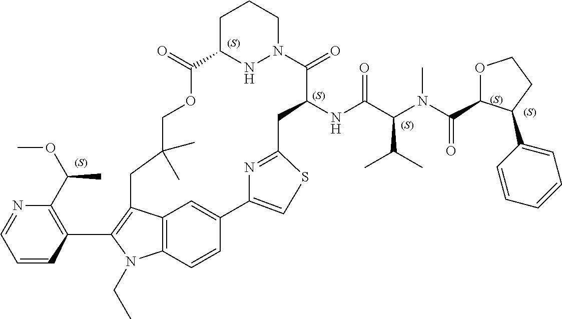 |
| AA429 | 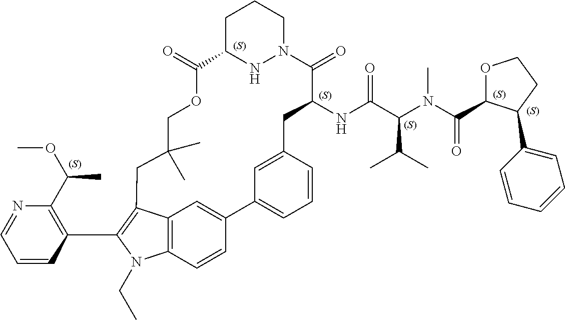 |
| AA430 | 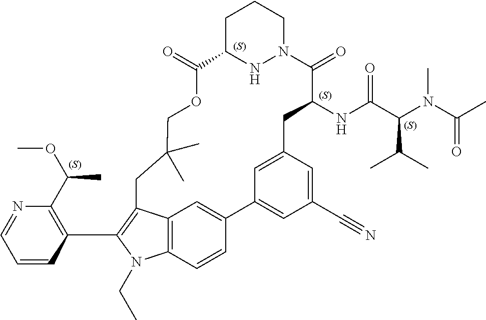 |
| AA431 | 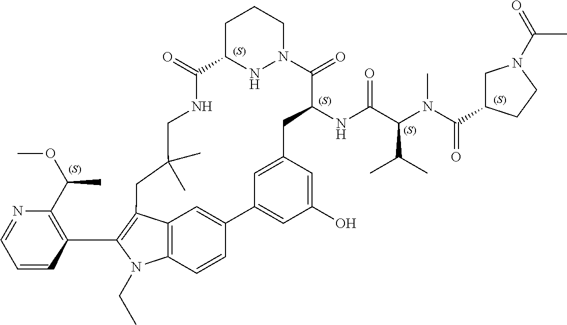 |
| AA432 | 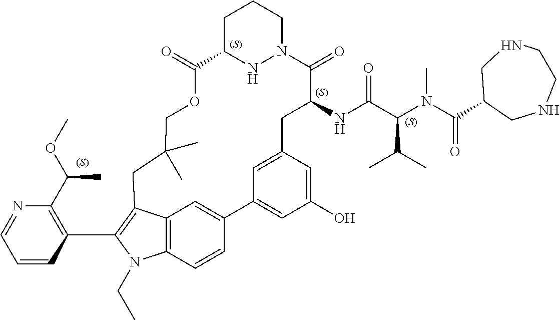 |
| AA433 | 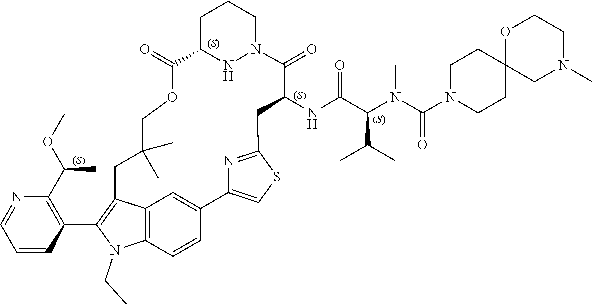 |
| AA434 | 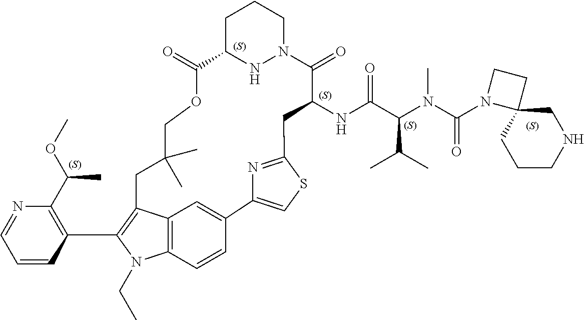 |
| AA435 | 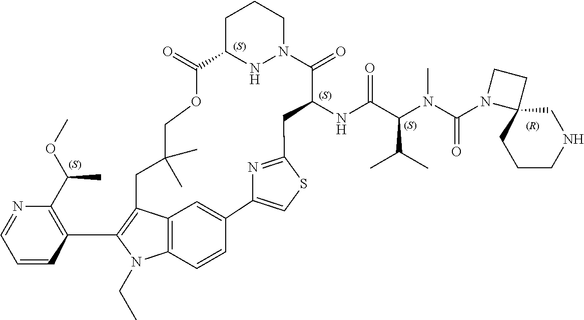 |
| AA436 | 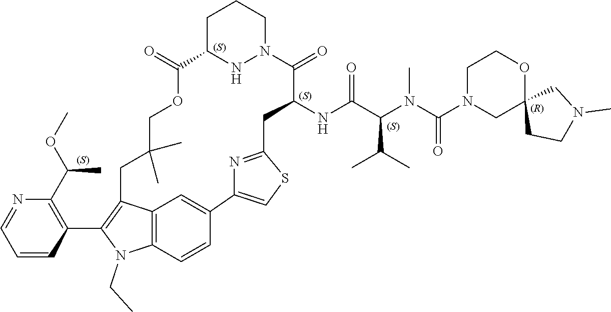 |
| AA437 | 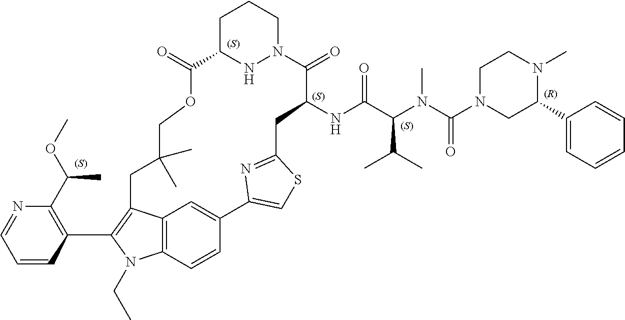 |
| AA438 | 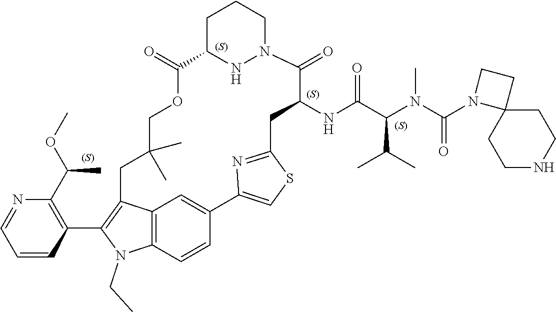 |
| AA439 | 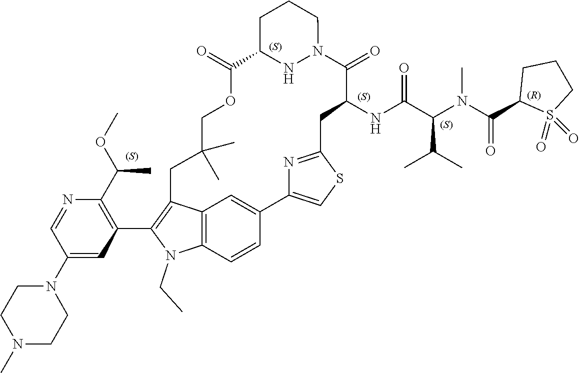 |
| AA440 | 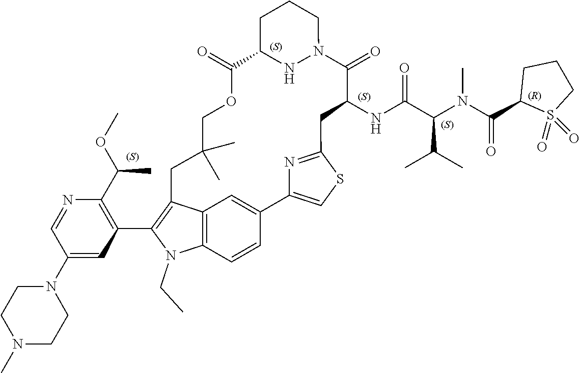 |
| AA441 | 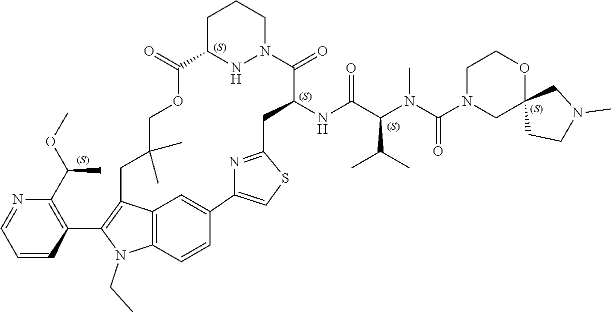 |
| AA442 | 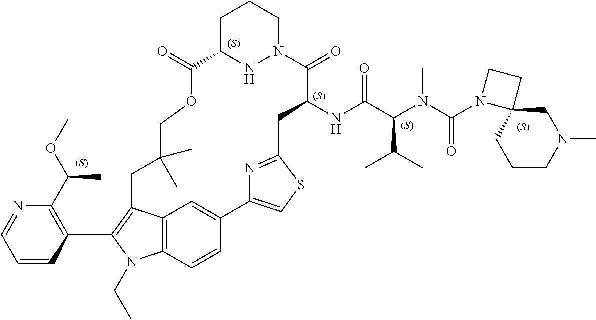 |
| AA443 | 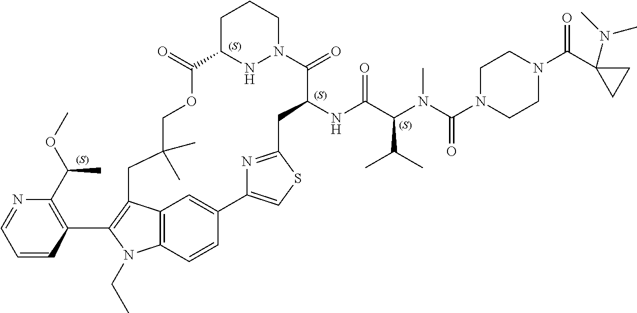 |
| AA444 | 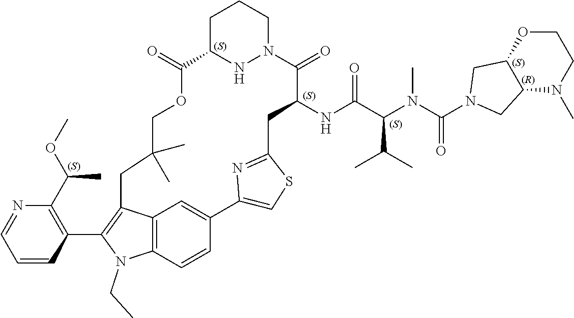 |
| AA445 | 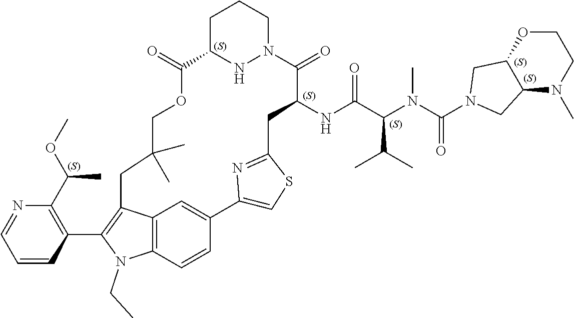 |
| AA446 | 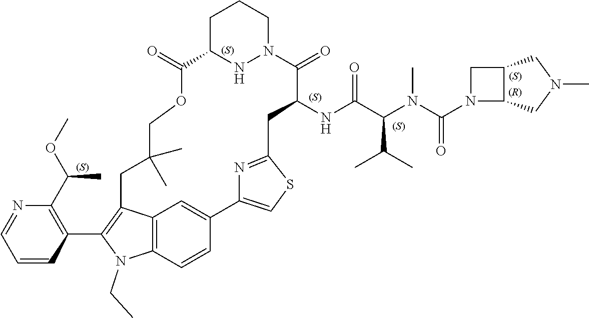 |
| AA447 | 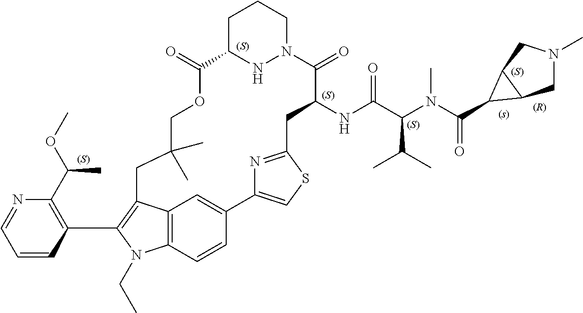 |
| AA448 | 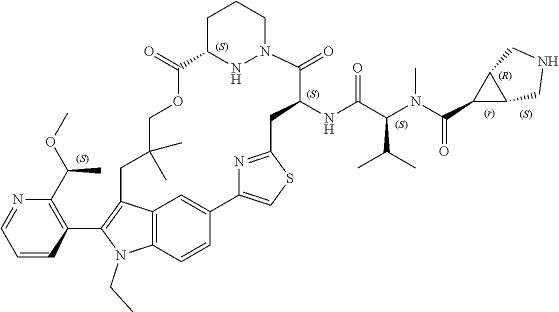 |
| AA449 | 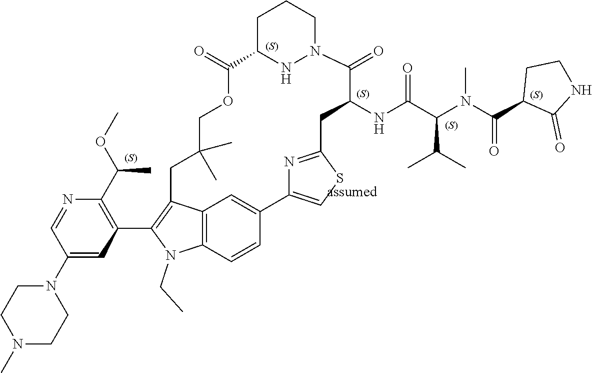 |
| AA450 | 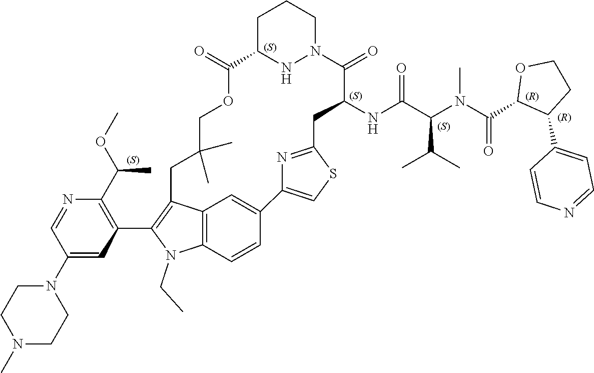 |
| AA451 | 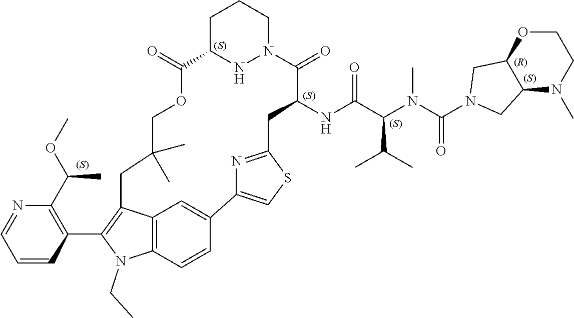 |
| AA452 | 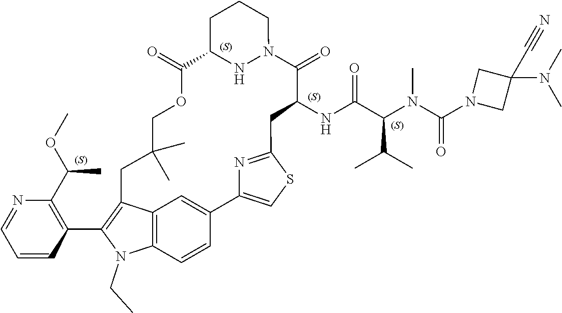 |
| AA453 | 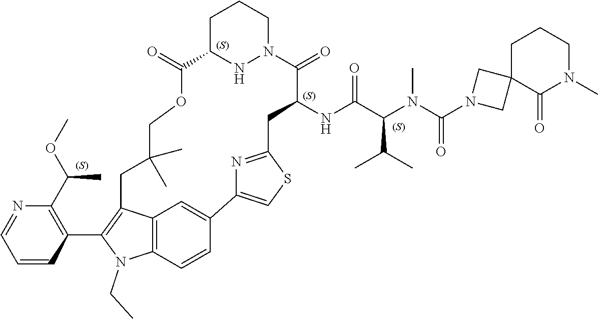 |
| AA454 | 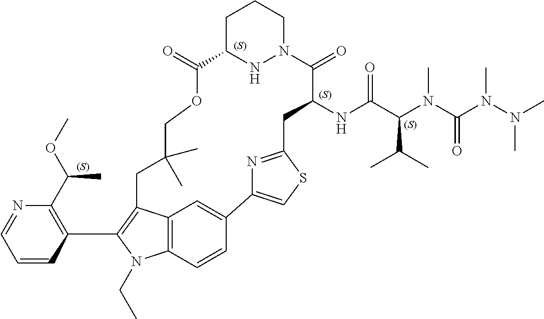 |
| AA455 | 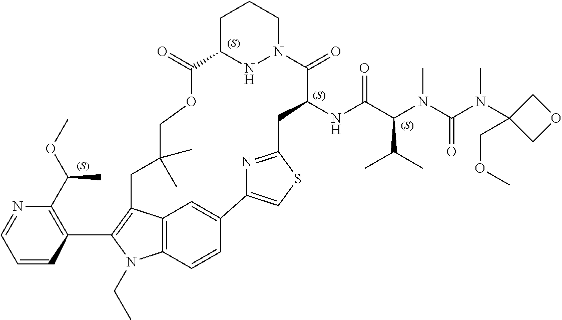 |
| AA456 | 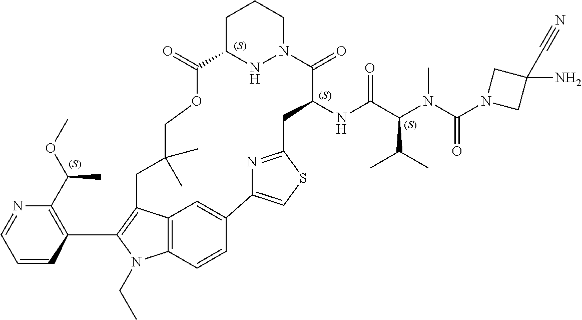 |
| AA457 | 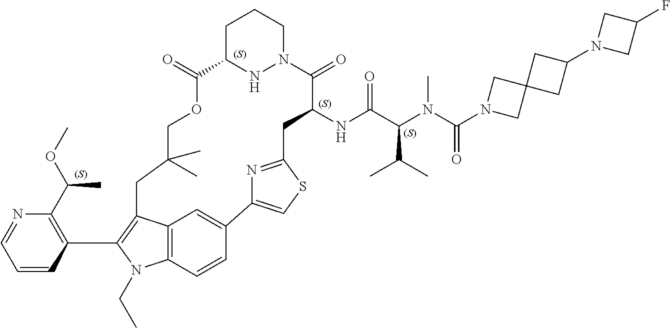 |
| AA458 | 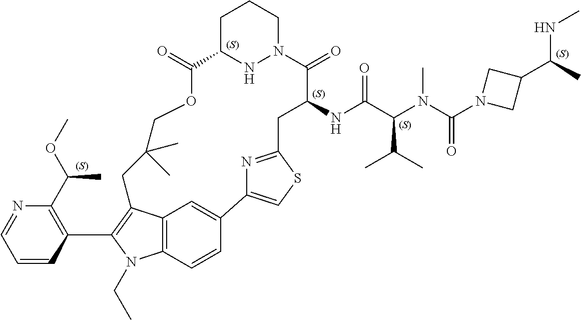 |
| AA459 | 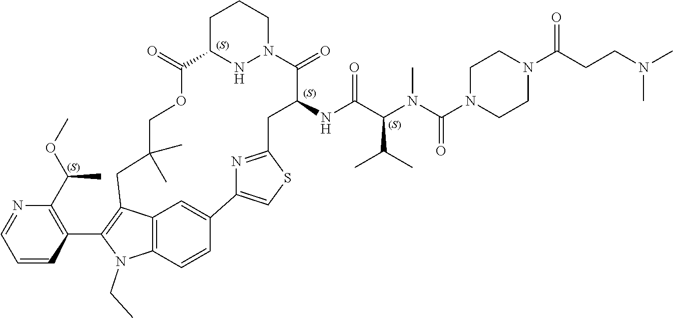 |
| AA460 | 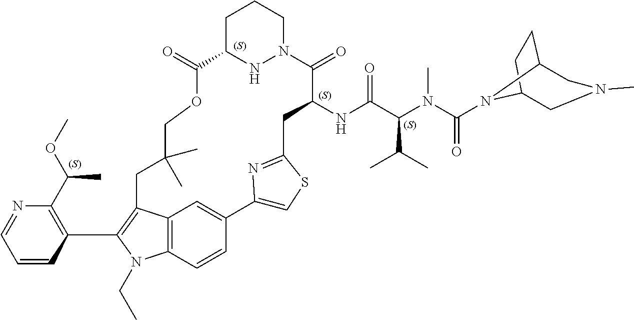 |
| AA461 | 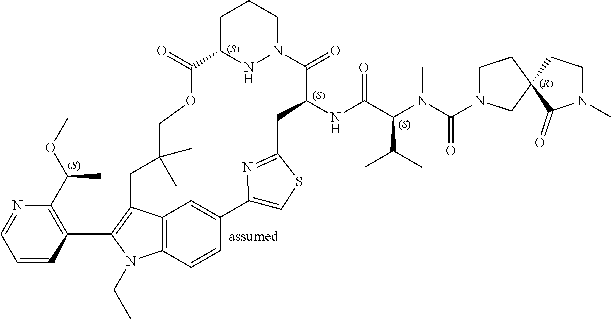 |
| AA462 | 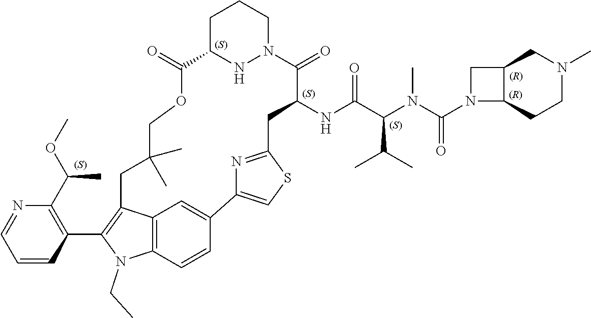 |
| AA463 | 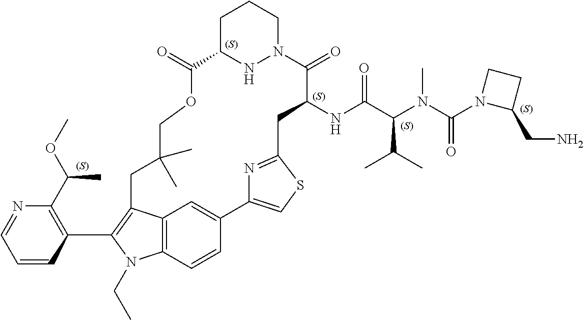 |
| AA464 | 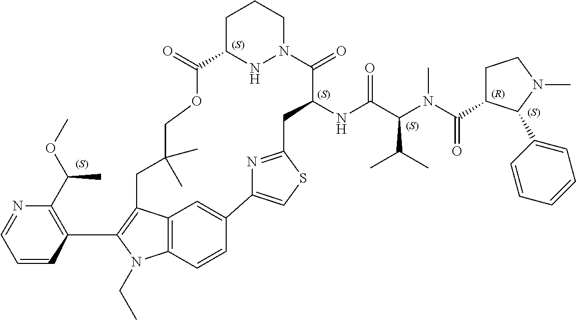 |
| AA465 | 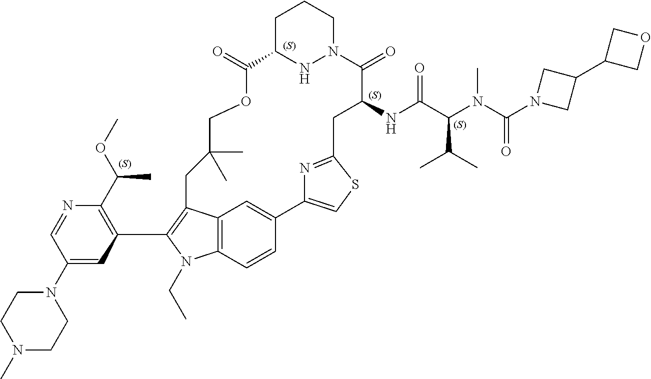 |
| AA466 | 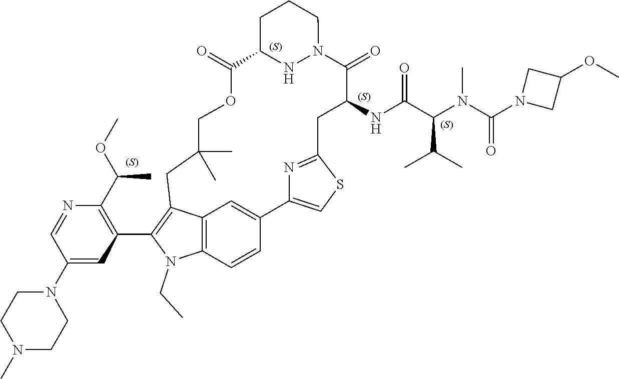 |
| AA467 | 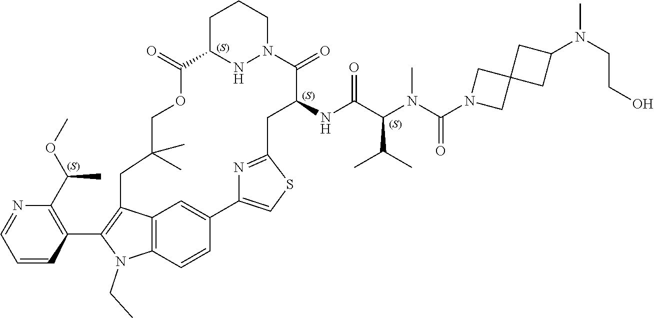 |
| AA468 | 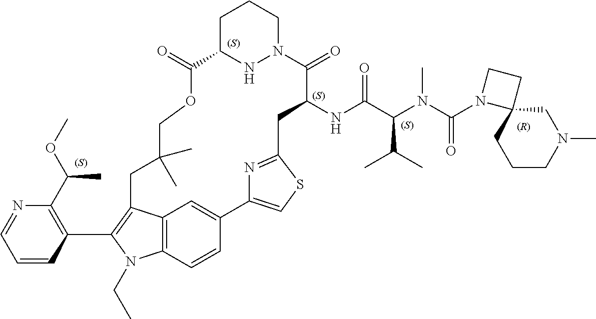 |
| AA469 | 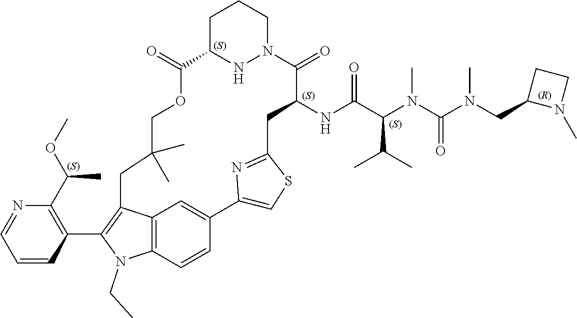 |
| AA470 | 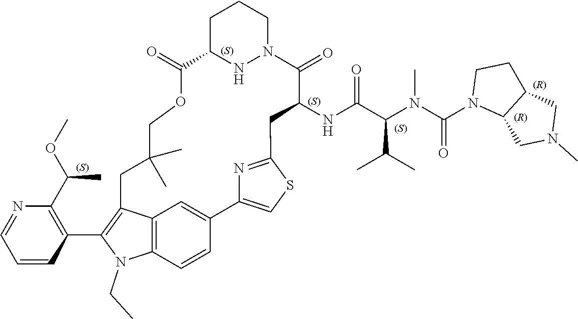 |
| AA471 | 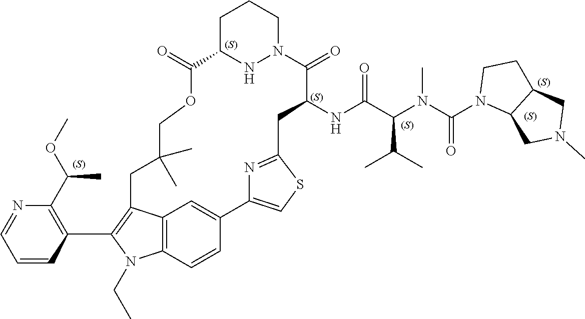 |
| AA472 | 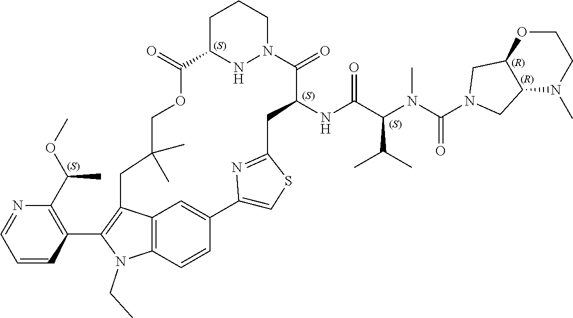 |
| AA473 | 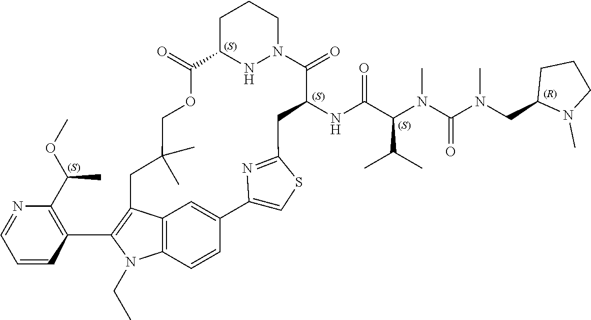 |
| AA474 | 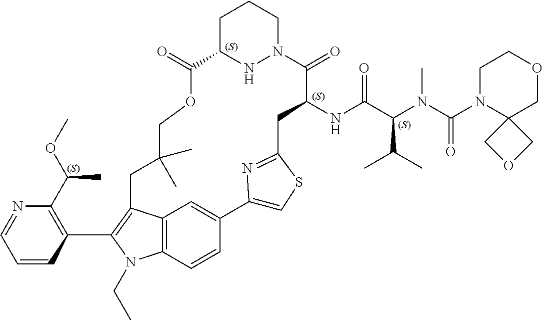 |
| AA475 | 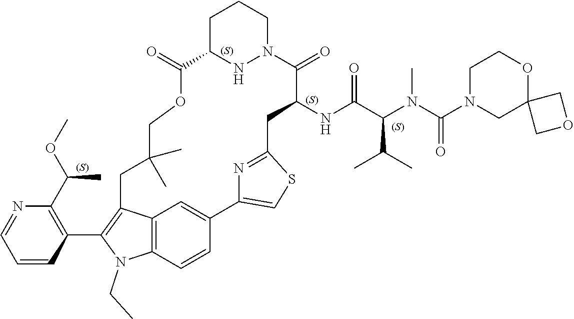 |
| AA476 | 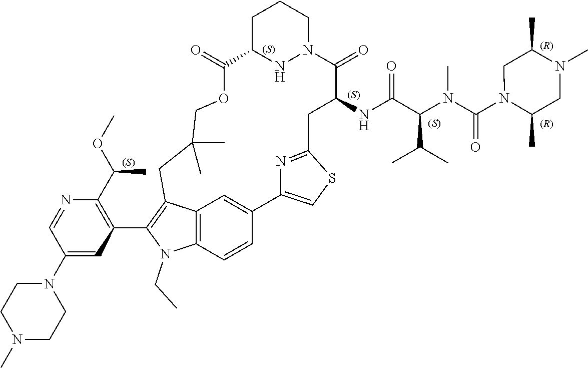 |
| AA477 | 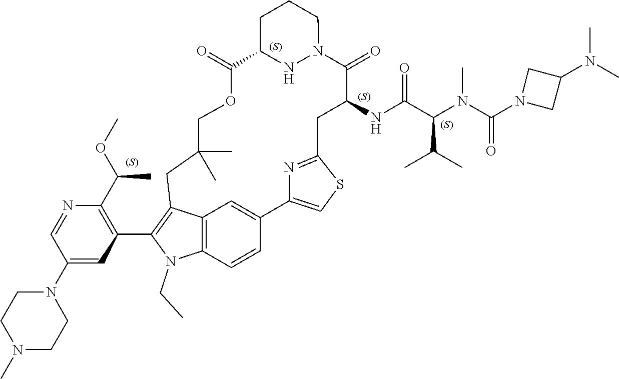 |
| AA478 | 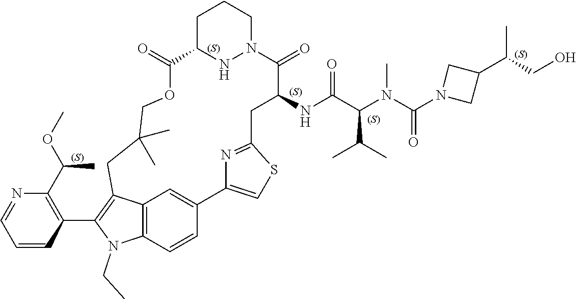 |
| AA479 | 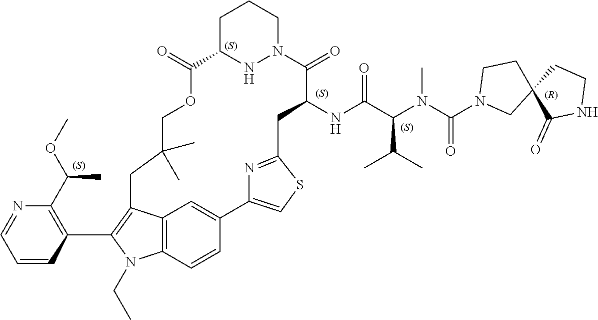 |
| AA480 | 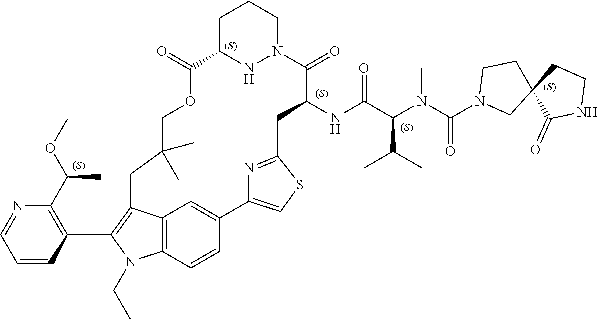 |
| AA481 | 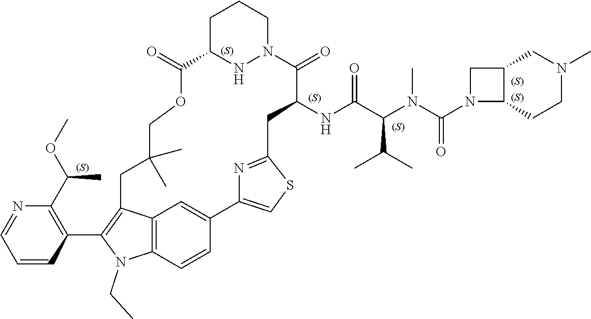 |
| AA482 | 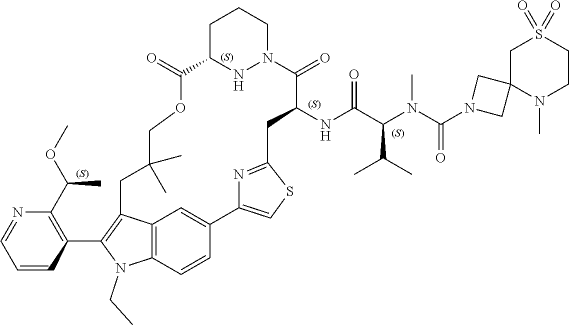 |
| AA483 | 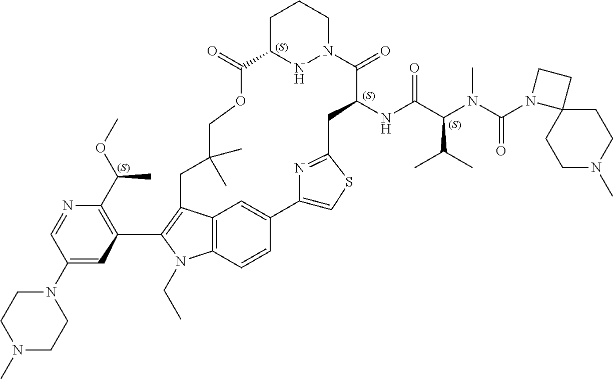 |
| AA484 | 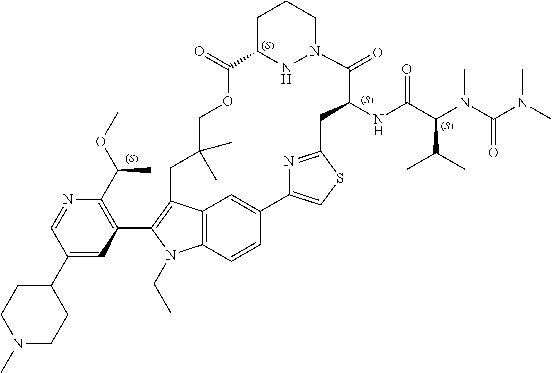 |
| AA485 | 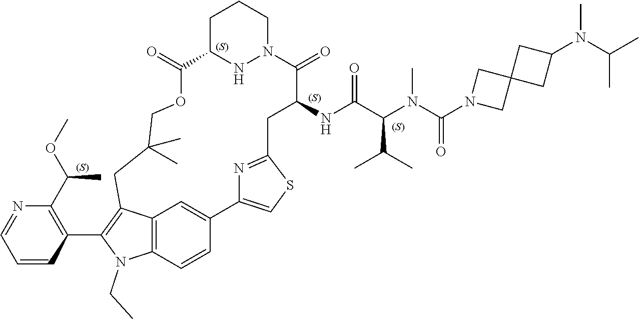 |
| AA486 | 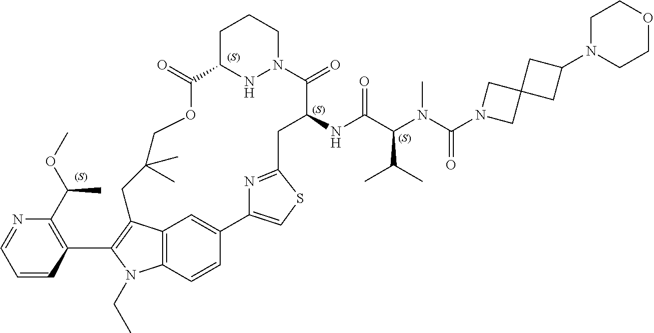 |
| AA487 | 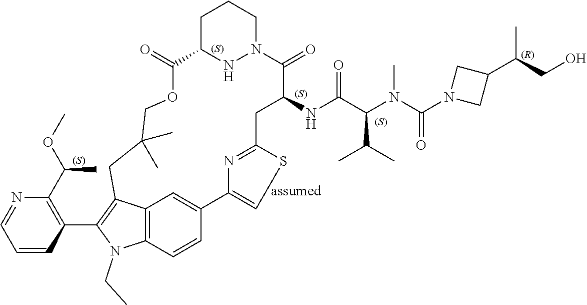 |
| AA488 | 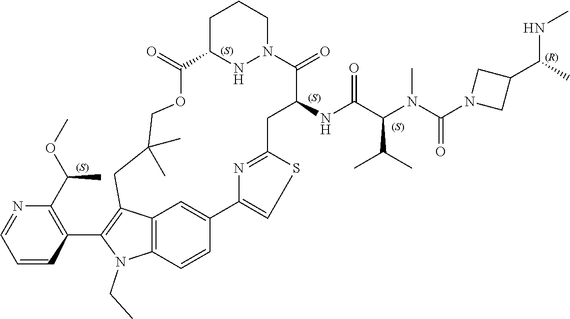 |
| AA489 | 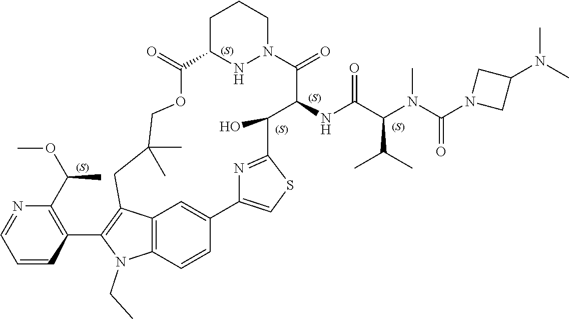 |
| AA490 | 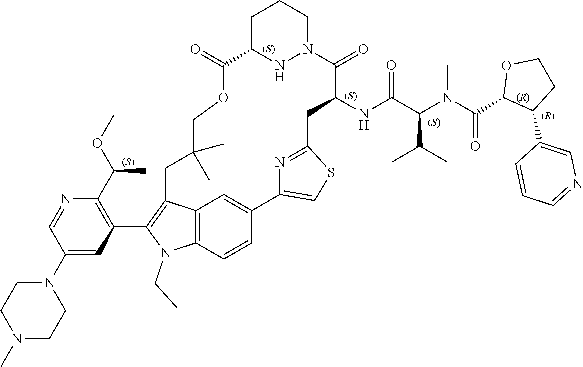 |
| AA491 | 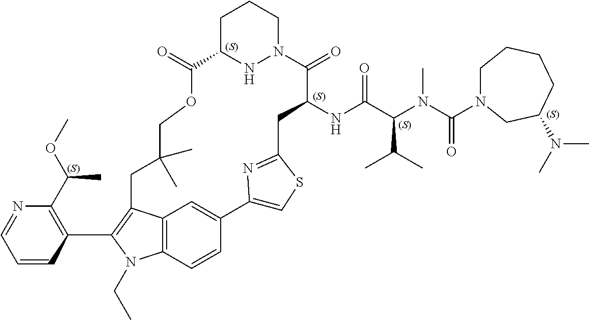 |
| AA492 | 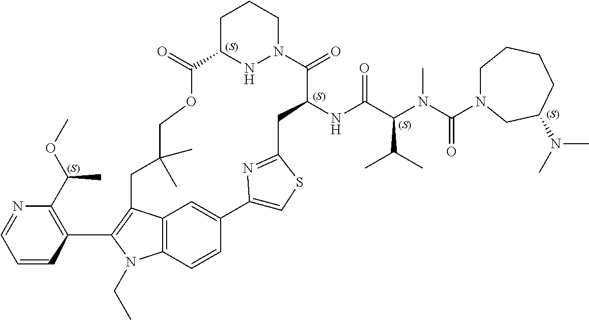 |
| AA493 | 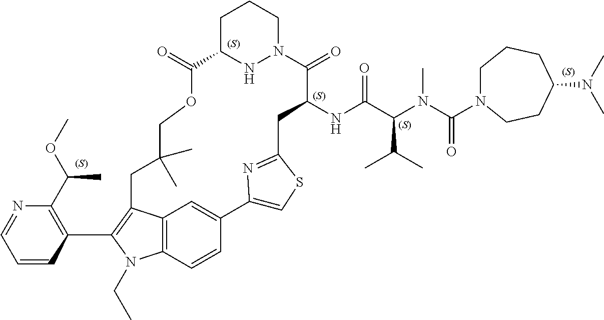 |
| AA494 | 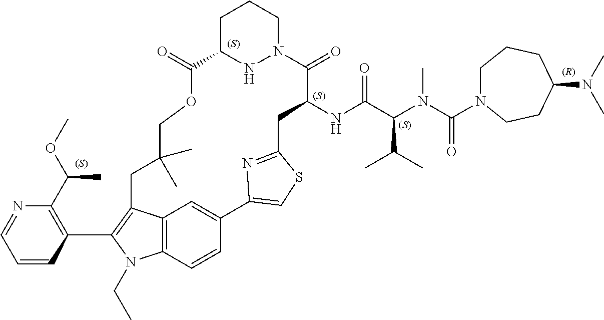 |
| AA495 | 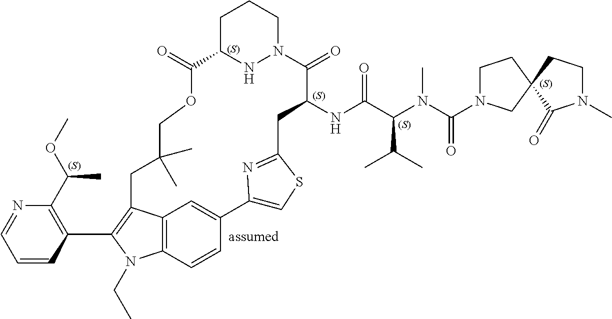 |
| AA496 | 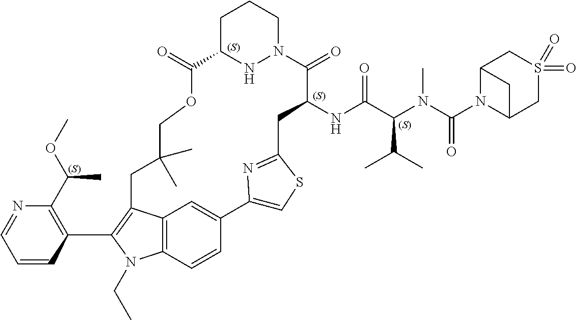 |
| AA497 | 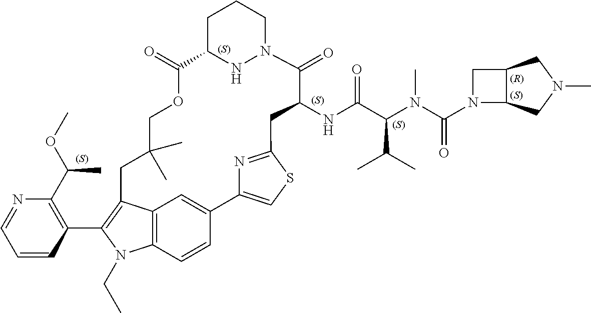 |
| AA498 | 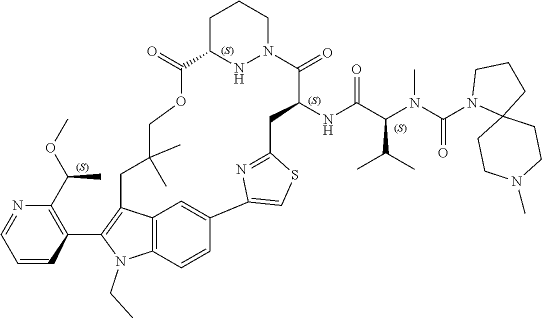 |
| AA499 | 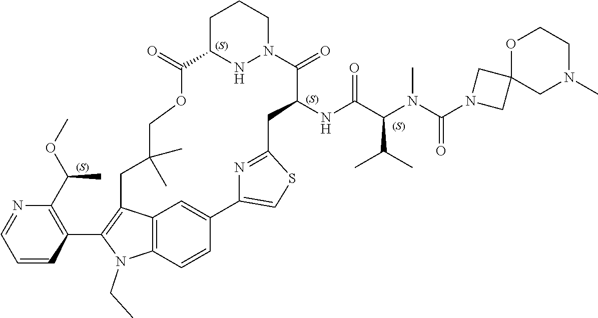 |
| AA500 | 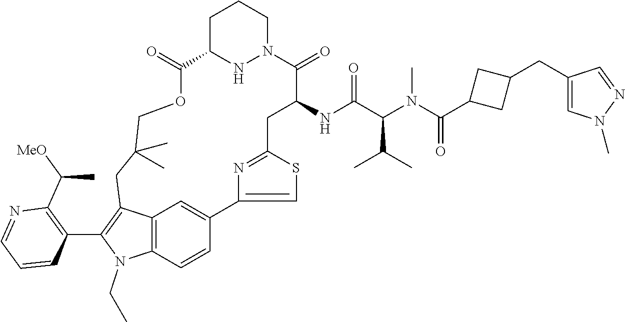 |
| AA501 | 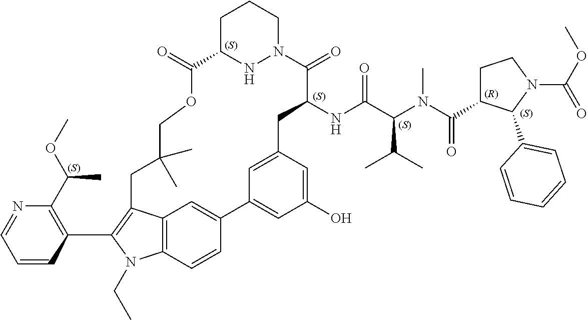 |
| AA502 | 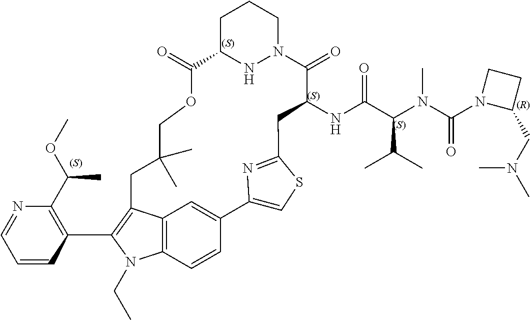 |
| AA503 | 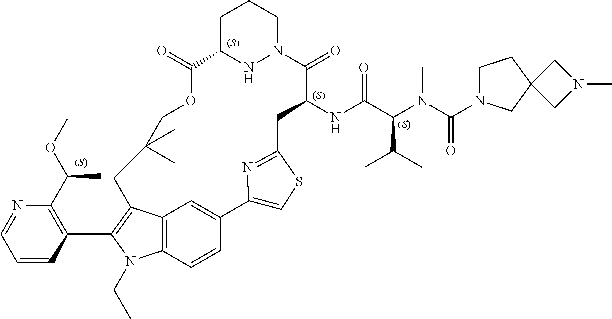 |
| AA504 | 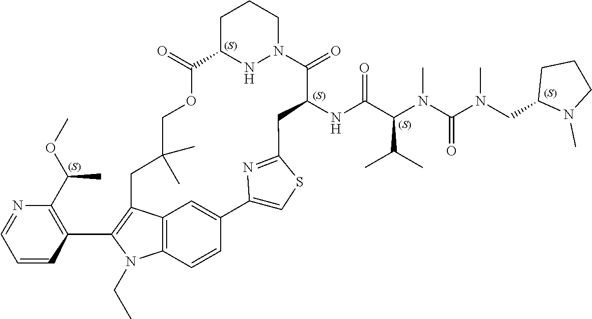 |
| AA505 | 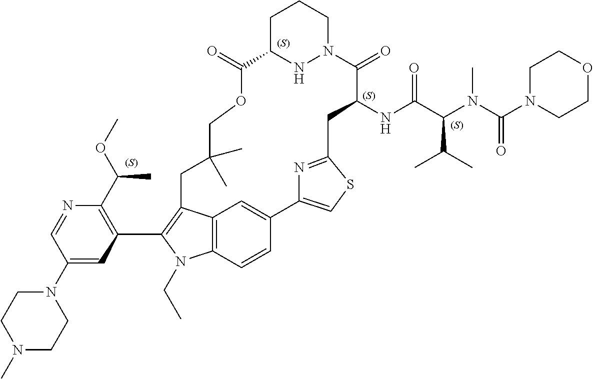 |
| AA506 | 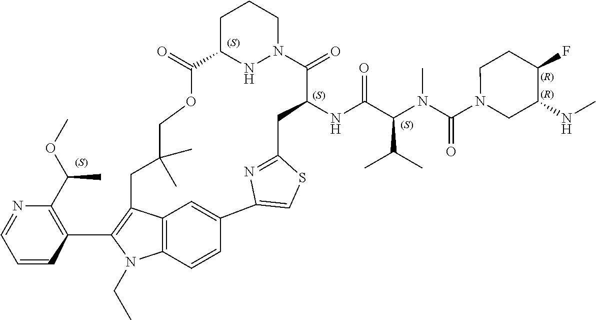 |
| AA507 | 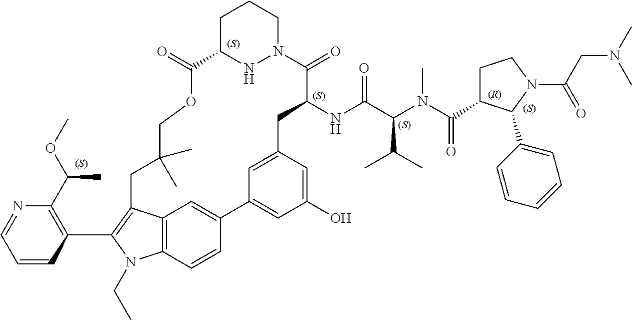 |
| AA508 | 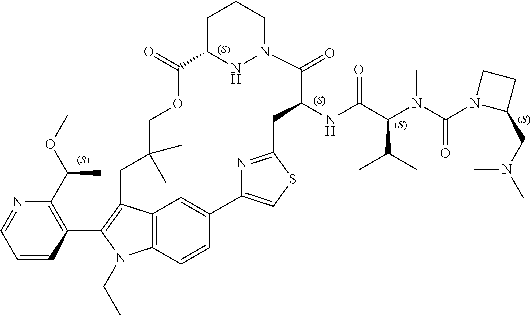 |
| AA509 | 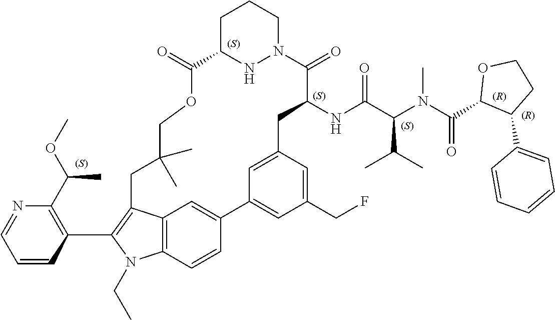 |
| AA510 | 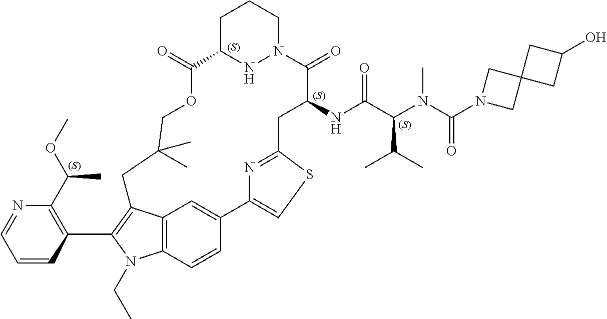 |
| AA511 | 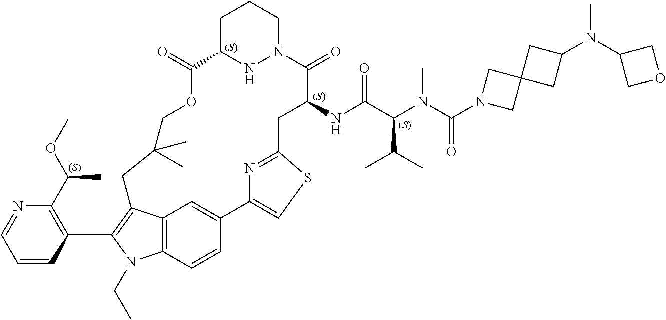 |
| AA512 | 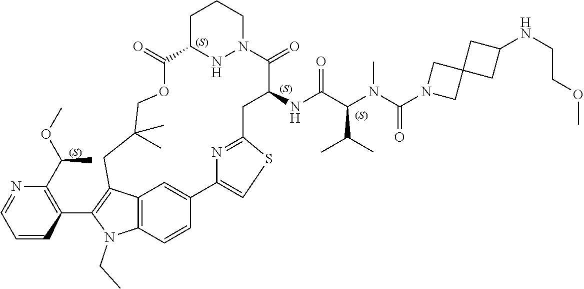 |
| AA513 | 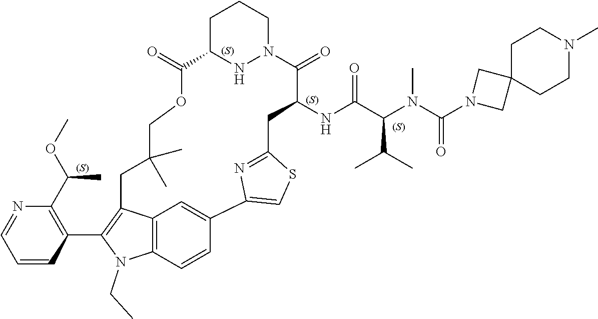 |
| AA514 | 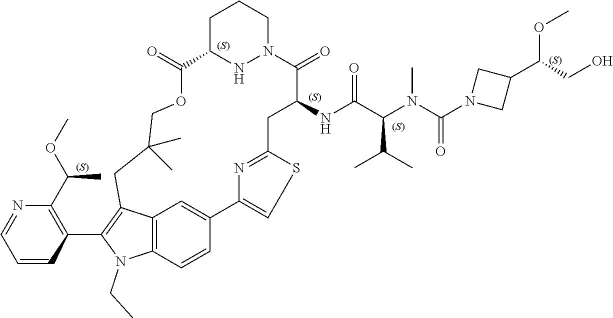 |
| AA515 | 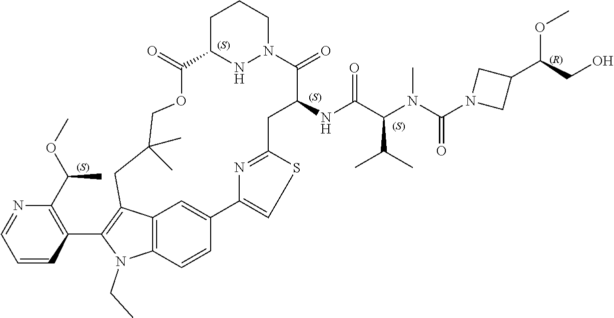 |
| AA516 | 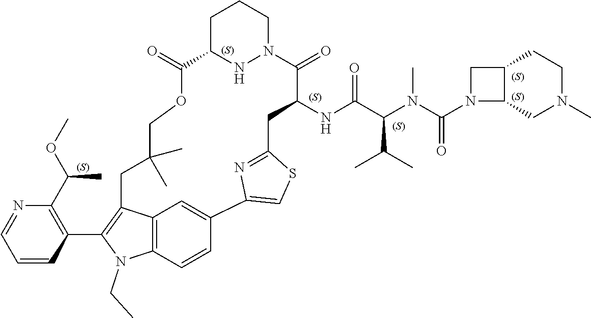 |
| AA517 | 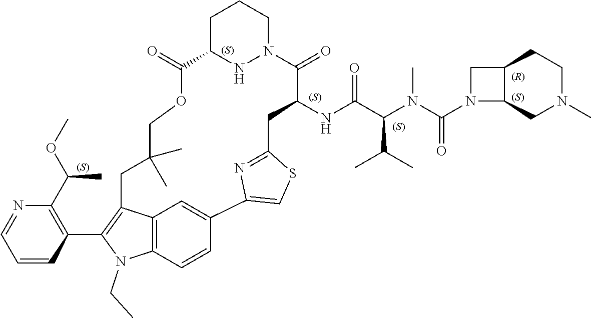 |
| AA518 | 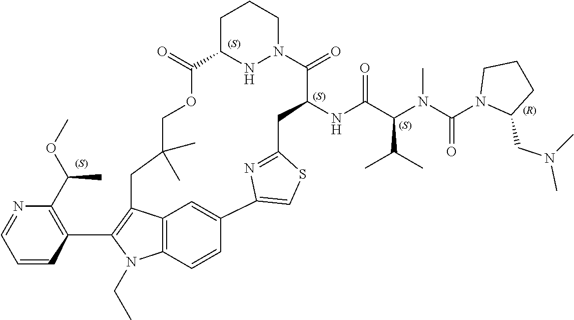 |
| AA519 | 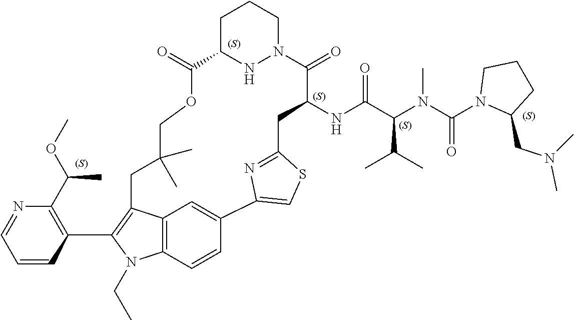 |
| AA520 | 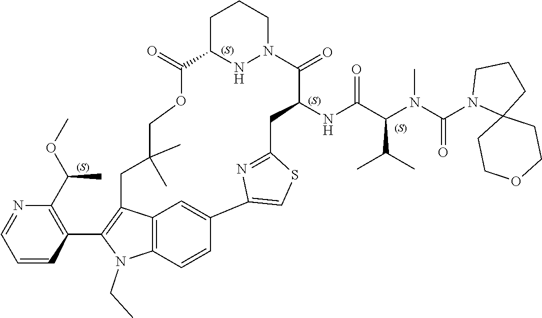 |
| AA521 | 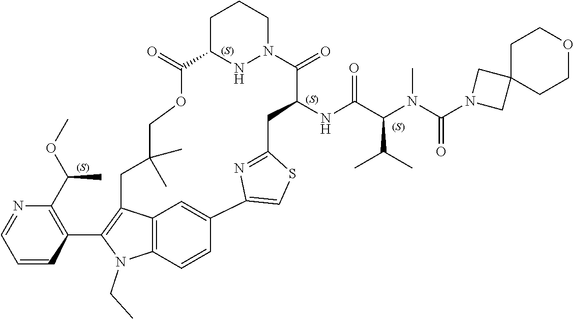 |
| AA522 | 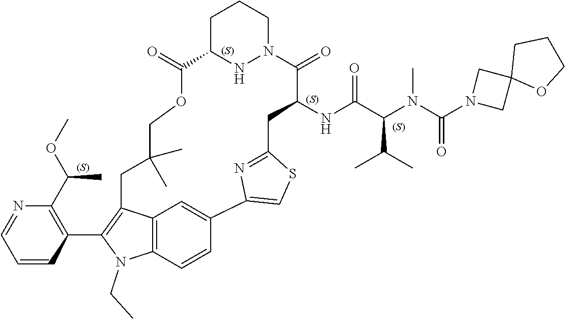 |
| AA523 | 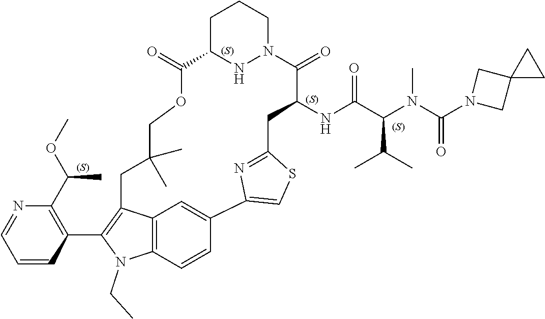 |
| AA524 | 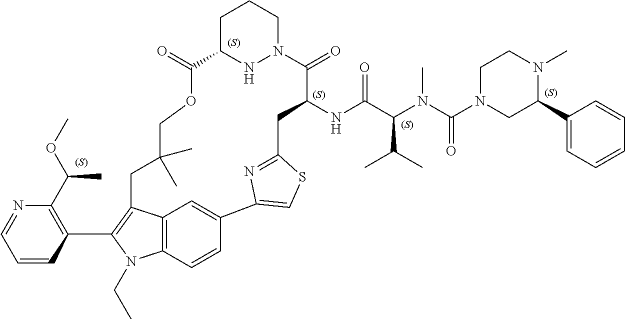 |
| AA525 | 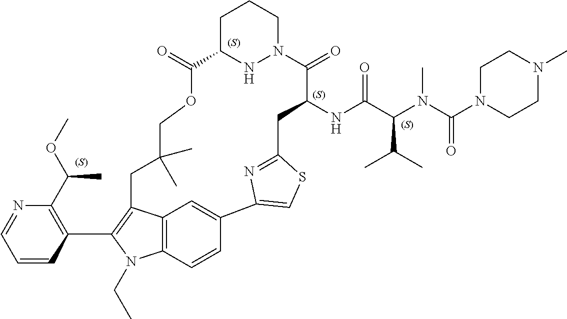 |
| AA526 | 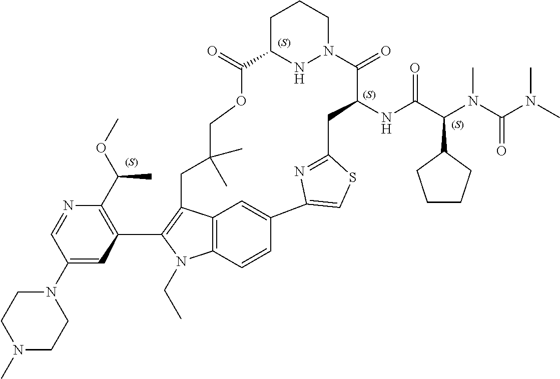 |
| AA527 | 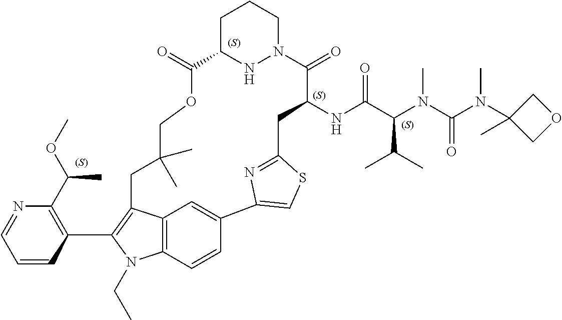 |
| AA528 | 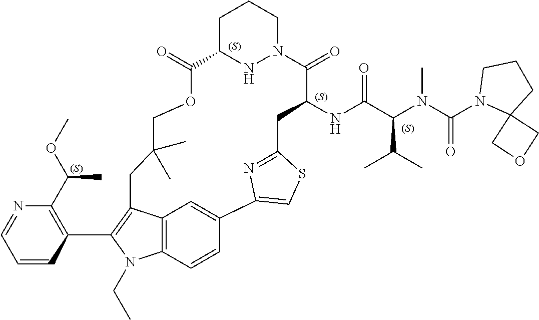 |
| AA529 | 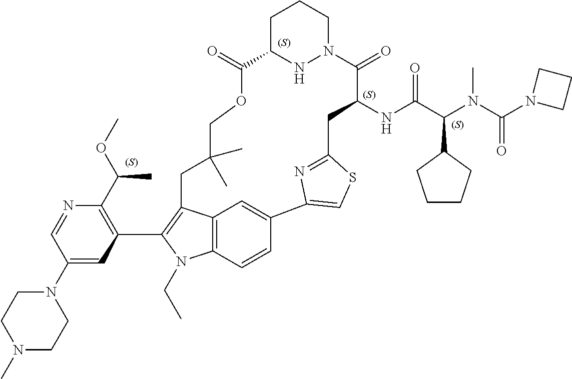 |
| AA530 | 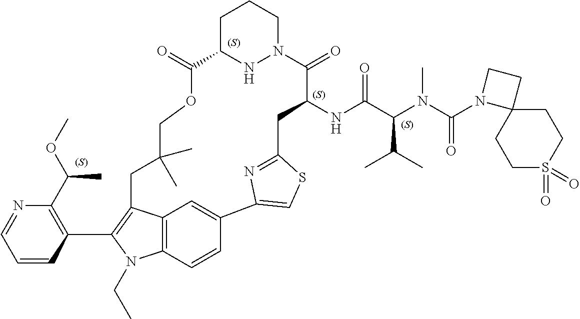 |
| AA531 | 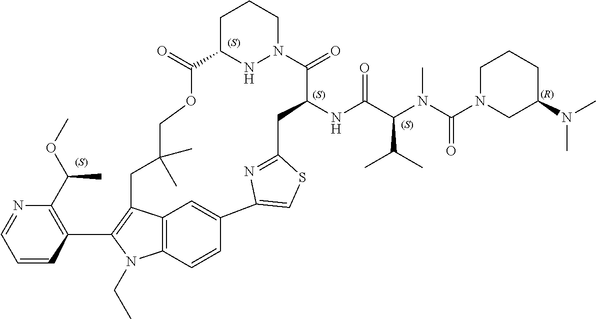 |
| AA532 | 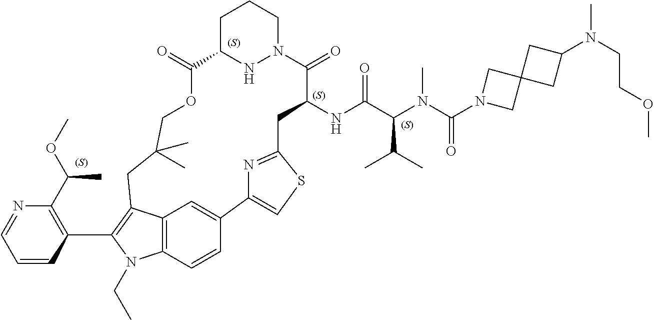 |
| AA533 | 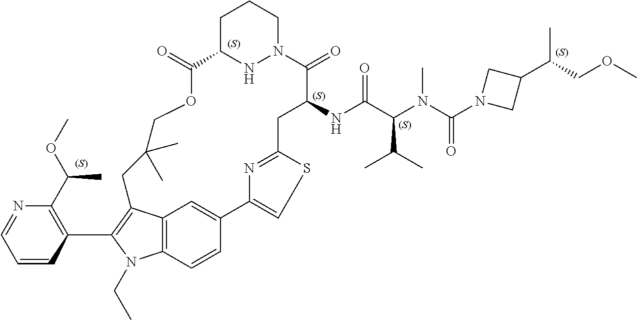 |
| AA534 | 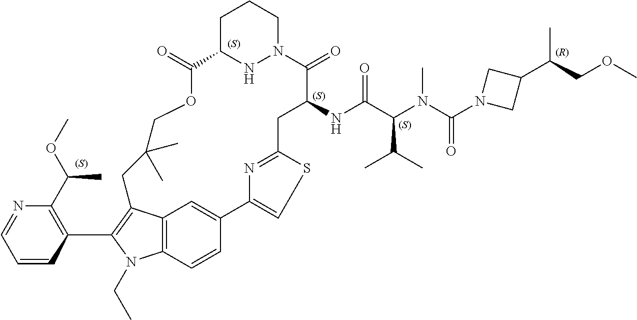 |
| AA535 | 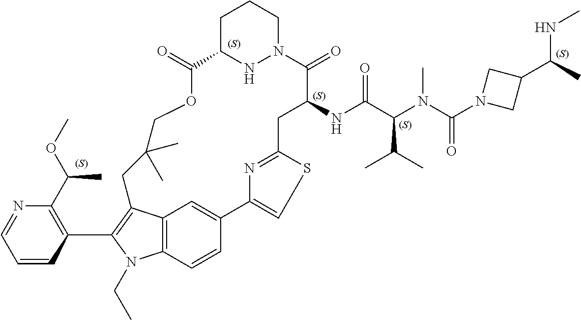 |
| AA536 | 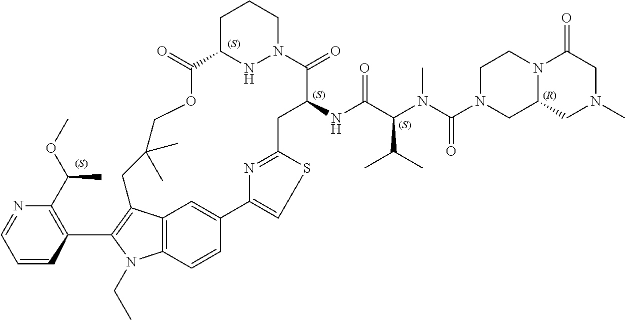 |
| AA537 | 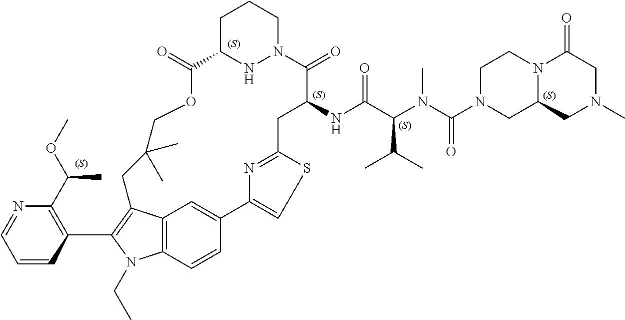 |
| AA538 | 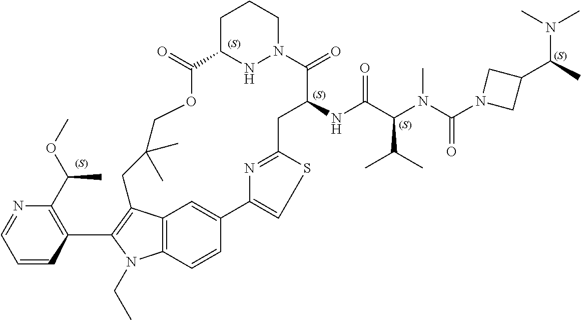 |
| AA539 | 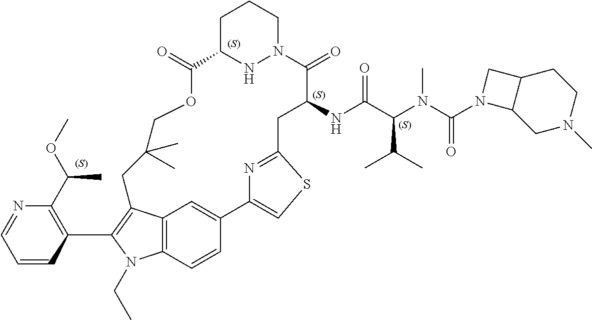 |
| AA540 | 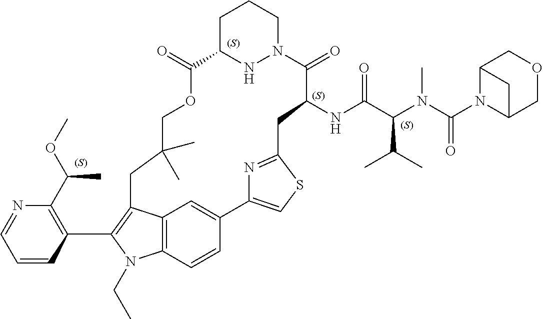 |
| AA541 | 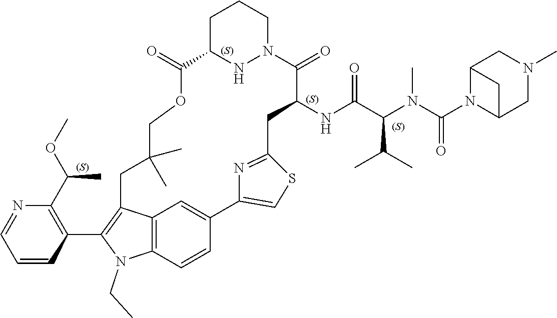 |
| AA542 | 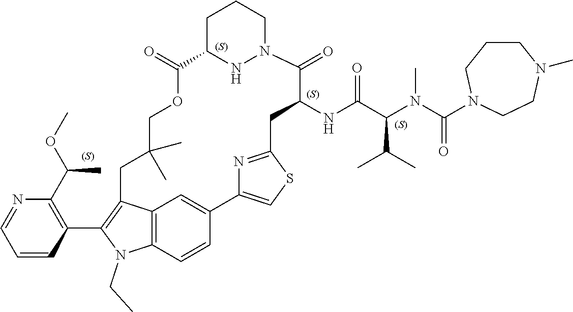 |
| AA543 | 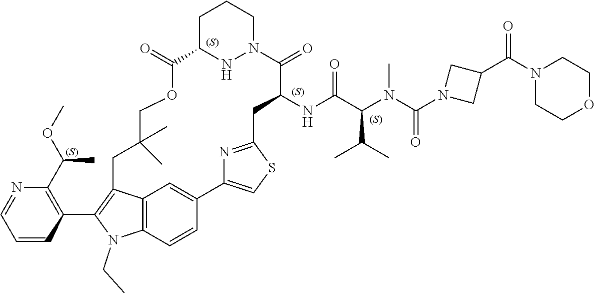 |
| AA544 | 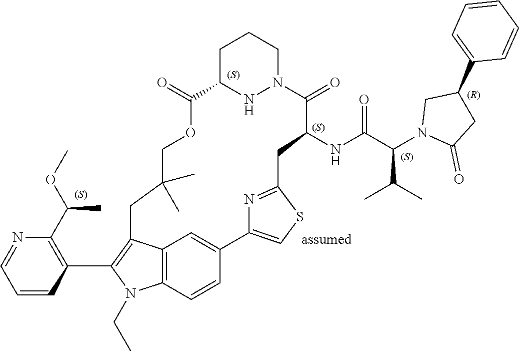 |
| AA545 | 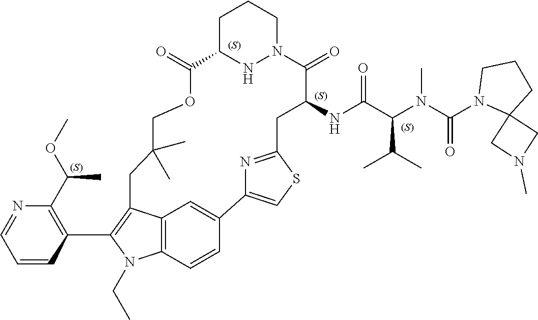 |
| AA546 | 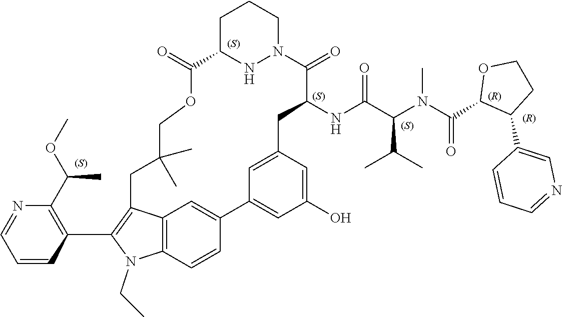 |
| AA547 | 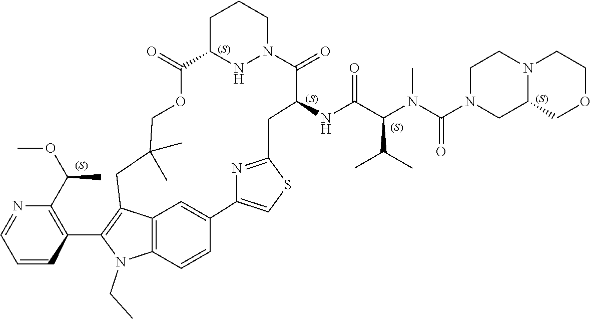 |
| AA548 | 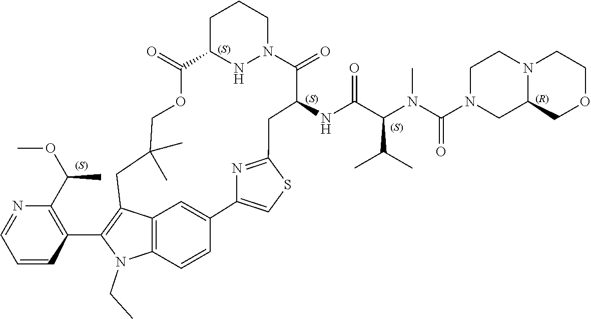 |
| AA549 | 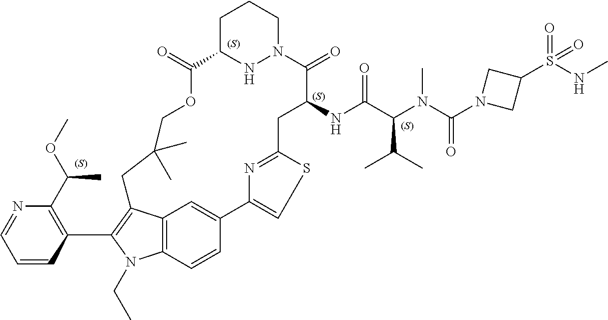 |
| AA550 | 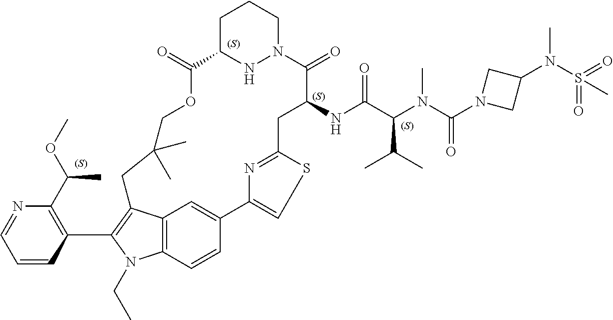 |
| AA551 | 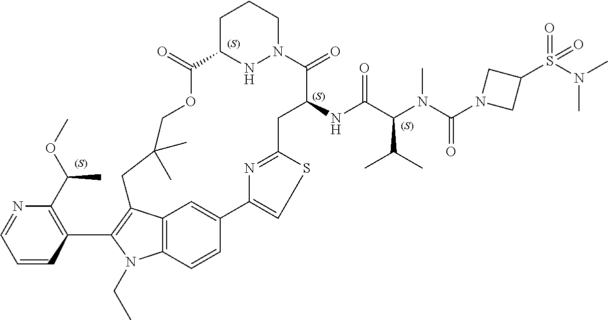 |
| AA552 | 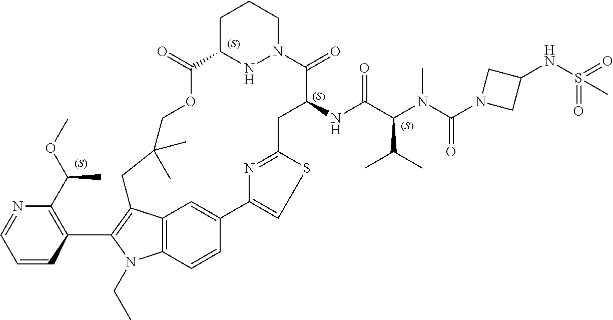 |
| AA553 | 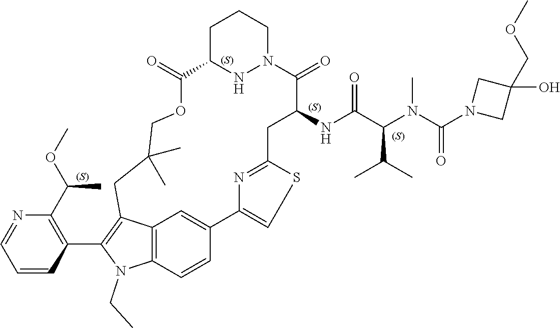 |
| AA554 | 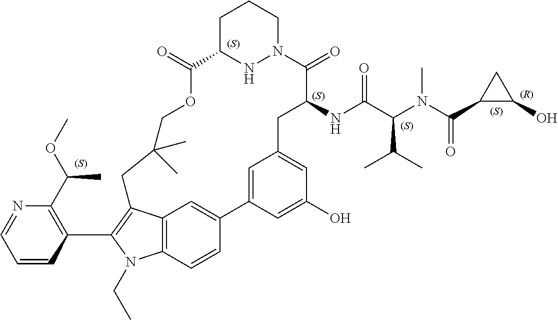 |
| AA555 | 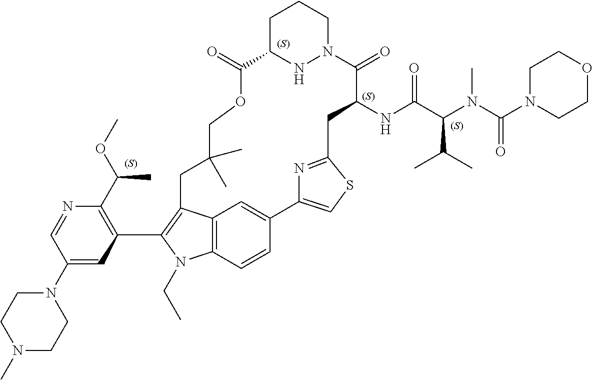 |
| AA556 | 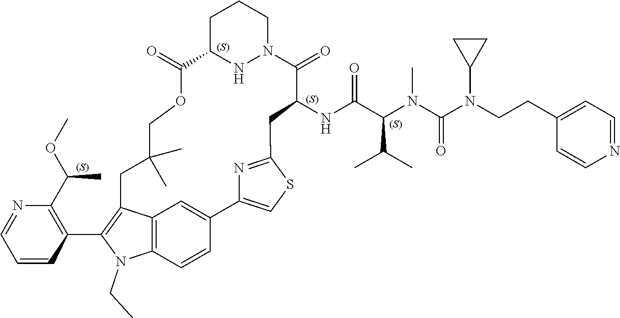 |
| AA557 | 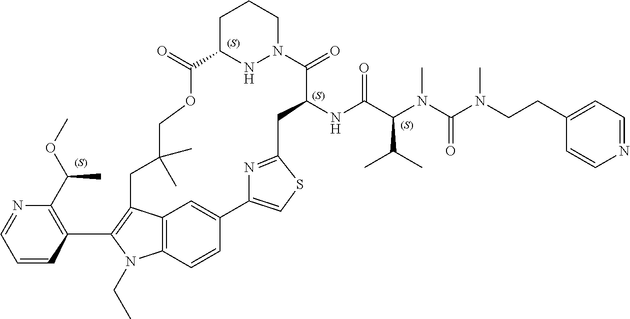 |
| AA558 | 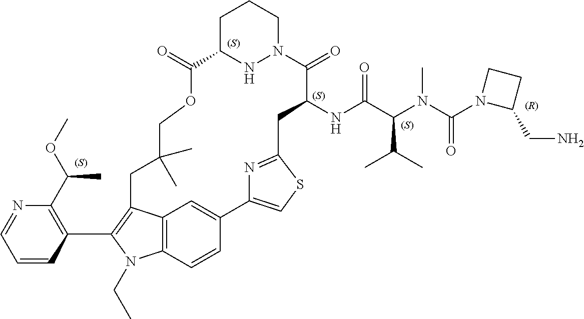 |
| AA559 | 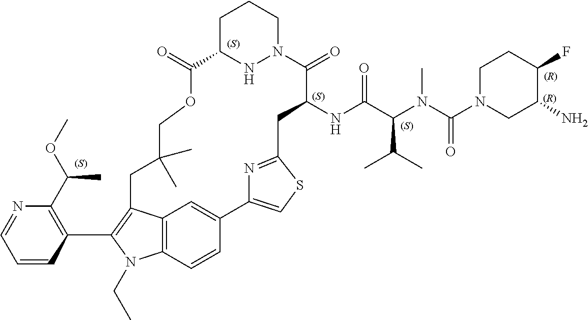 |
| AA560 | 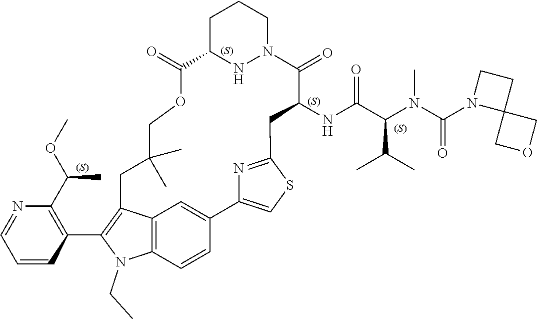 |
| AA561 | 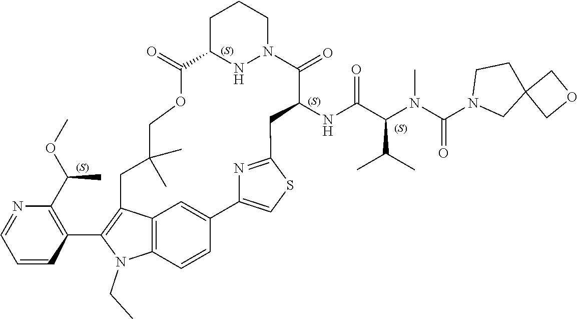 |
| AA562 | 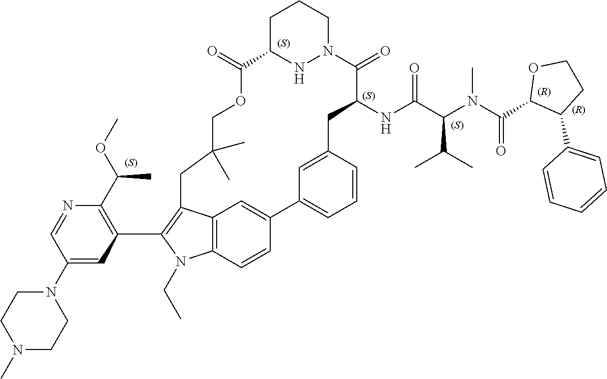 |
| AA563 | 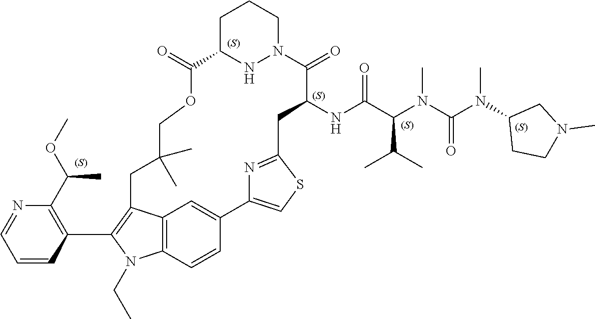 |
| AA564 | 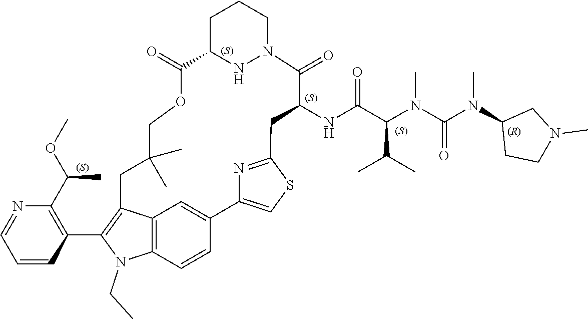 |
| AA565 | 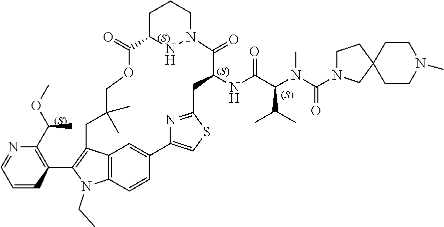 |
| AA566 | 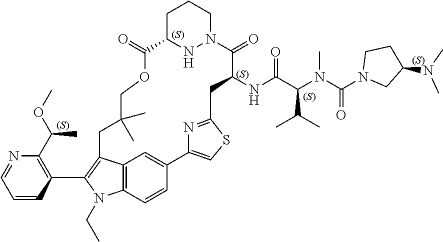 |
| AA567 | 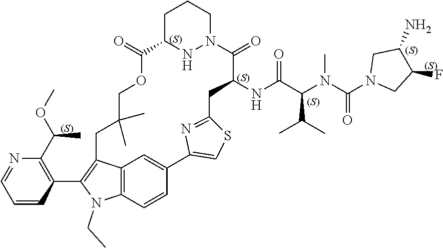 |
| AA568 | 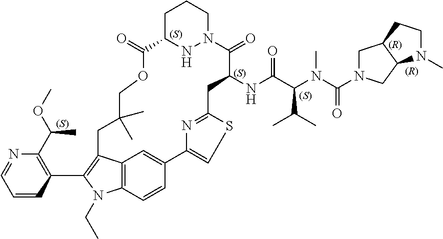 |
| AA569 | 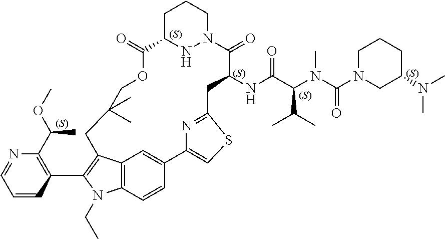 |
| AA570 | 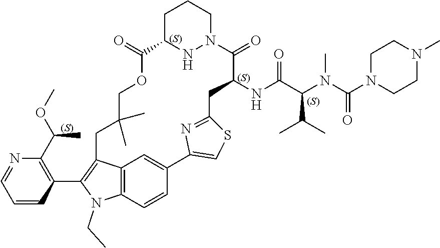 |
| AA571 | 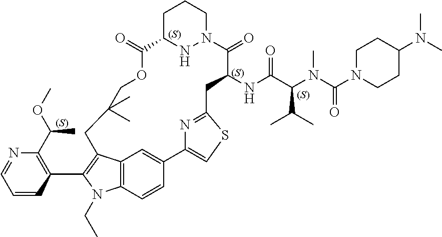 |
| AA572 | 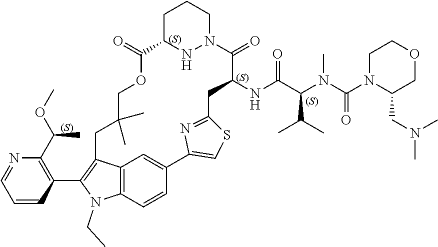 |
| AA573 | 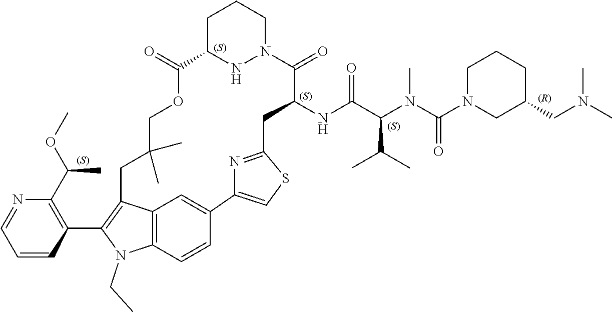 |
| AA574 | 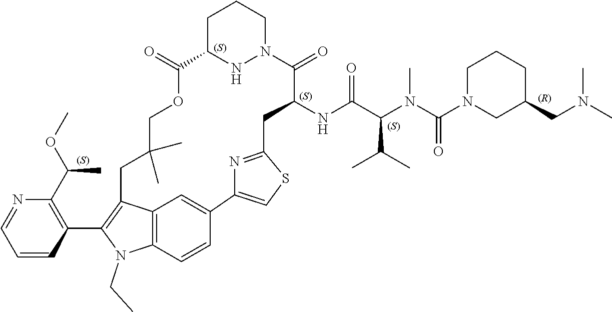 |
| AA575 | 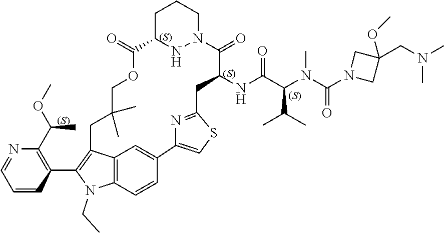 |
| AA576 | 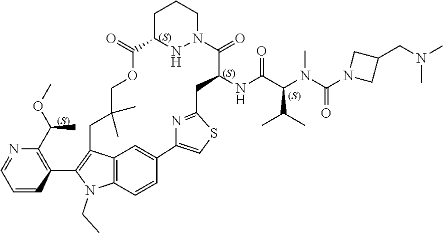 |
| AA577 | 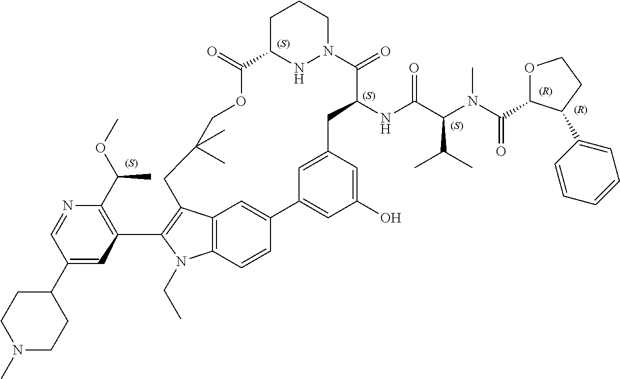 |
| AA578 | 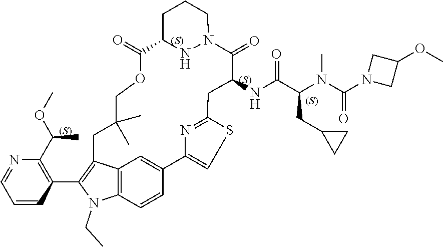 |
| AA579 | 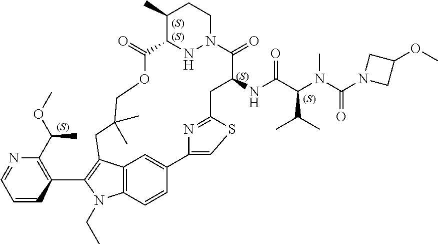 |
| AA580 | 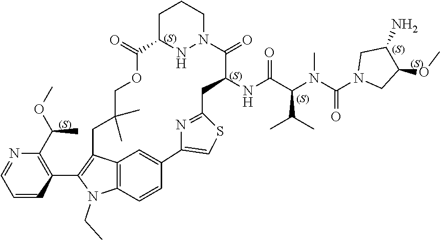 |
| AA581 | 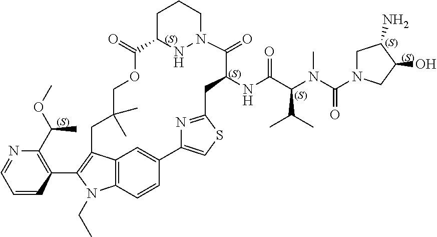 |
| AA582 | 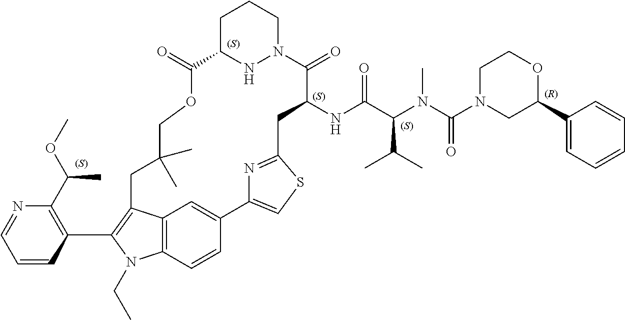 |
| AA583 | 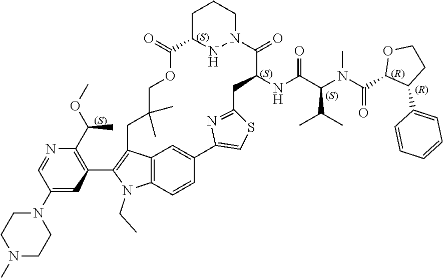 |
| AA584 | 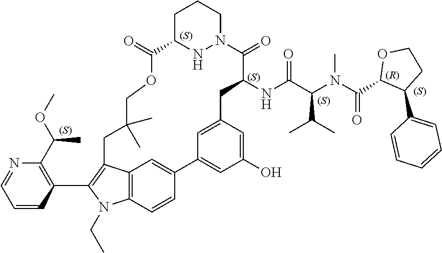 |
| AA585 | 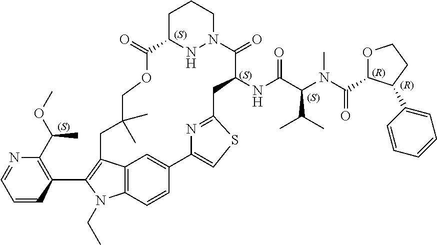 |
| AA586 | 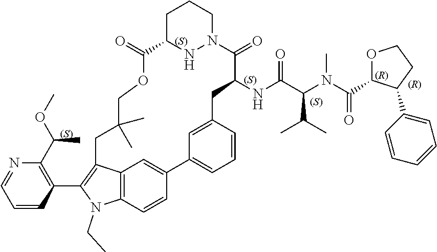 |
| AA587 | 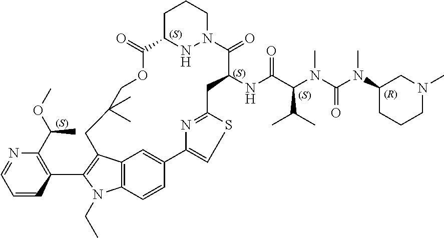 |
| AA588 | 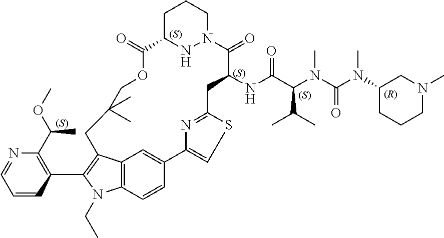 |
| AA589 | 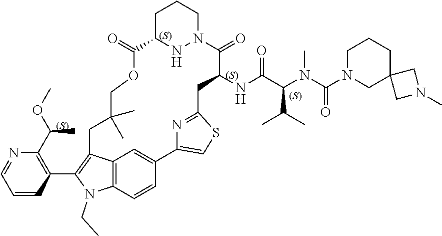 |
| AA590 | 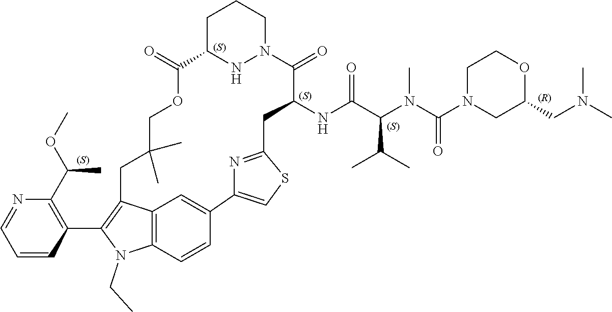 |
| AA591 | 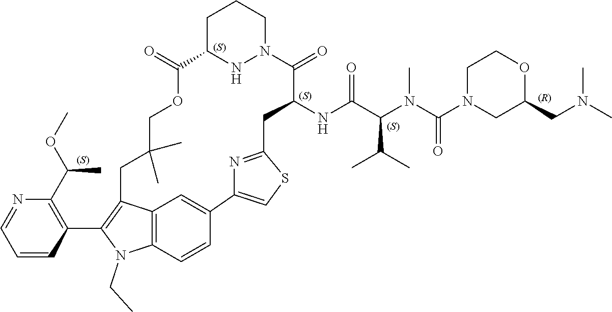 |
| AA592 | 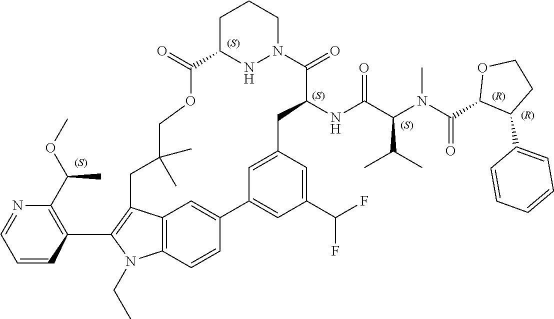 |
| AA593 | 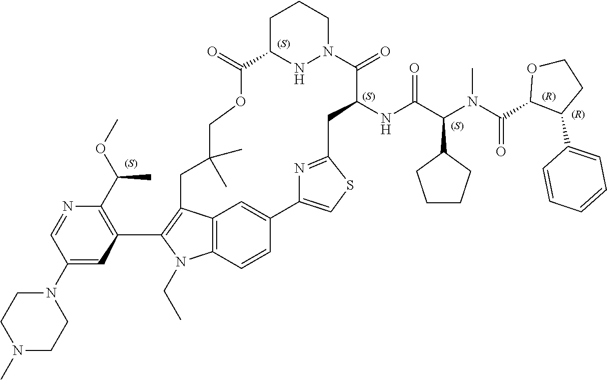 |
| AA594 | 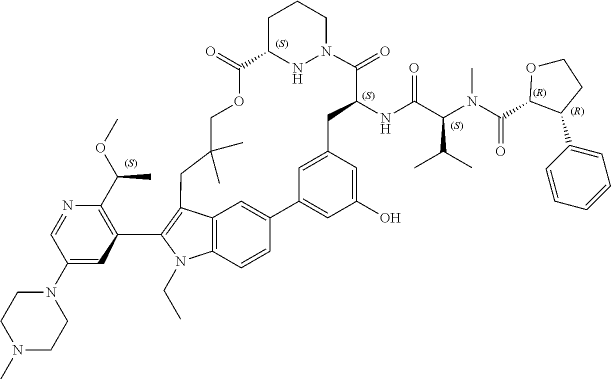 |
| AA595 | 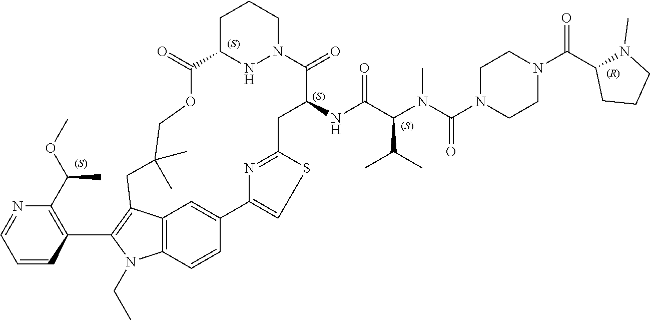 |
| AA596 | 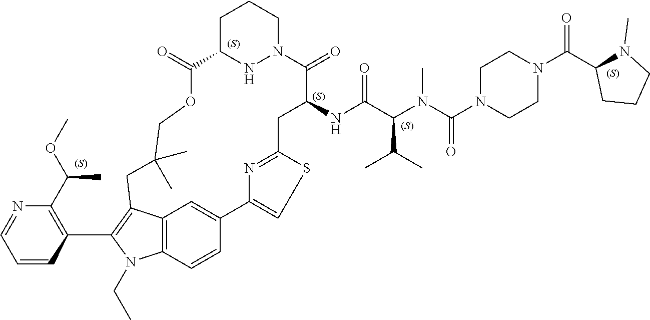 |
| AA597 | 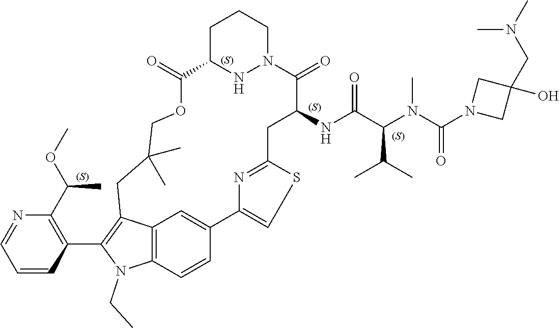 |
| AA598 | 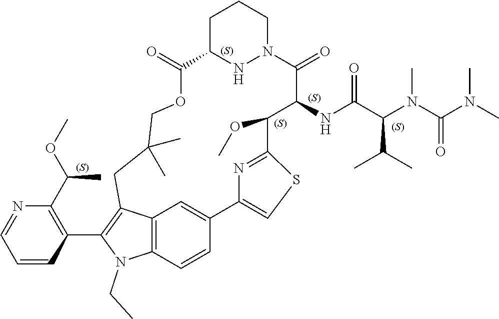 |
| AA599 | 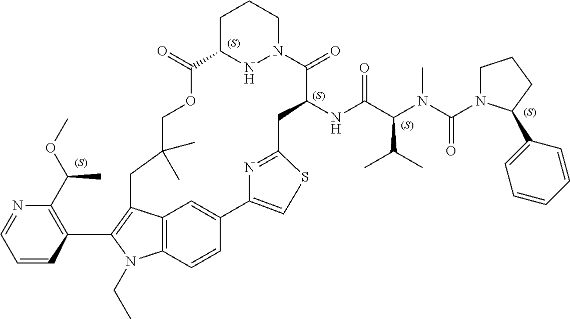 |
| AA600 | 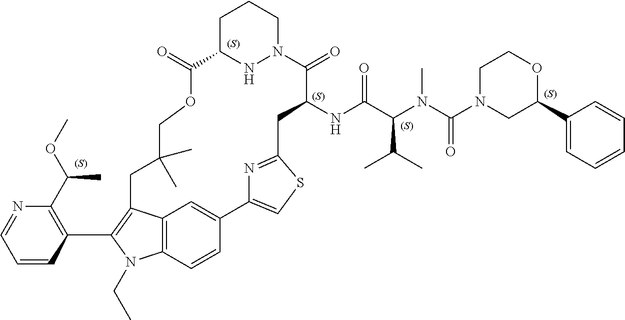 |
| AA601 | 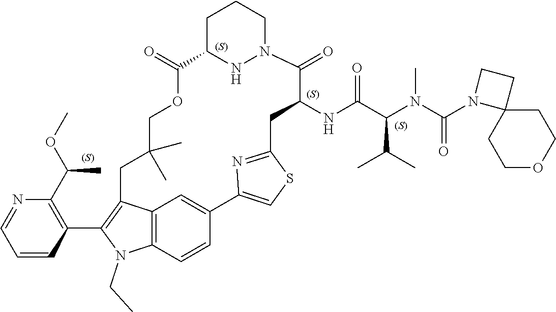 |
| AA602 | 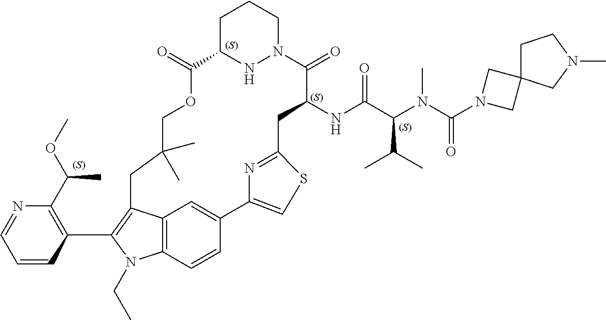 |
| AA603 | 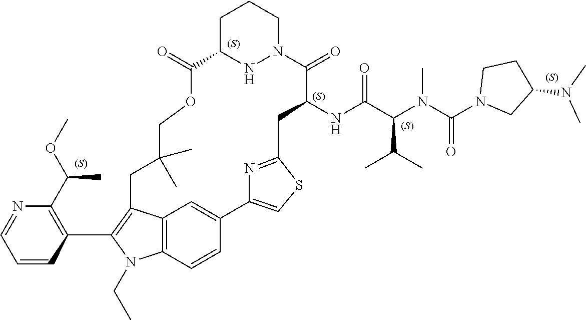 |
| AA604 | 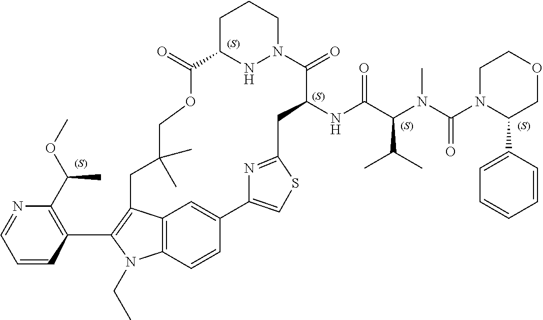 |
| AA605 | 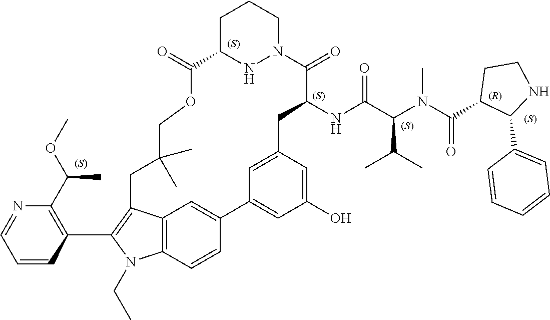 |
| AA606 | 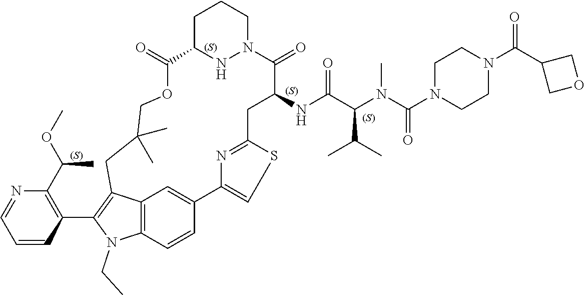 |
| AA607 | 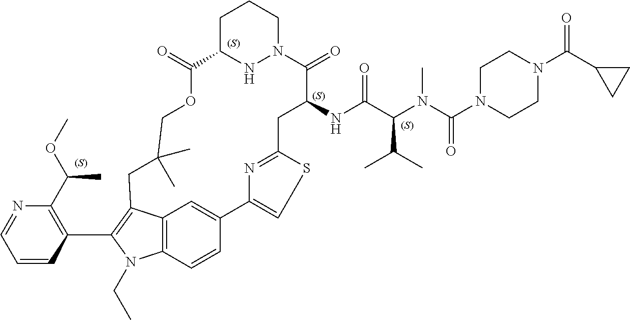 |
| AA608 | 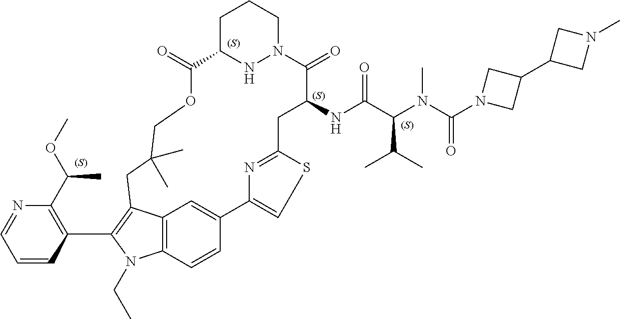 |
| AA609 | 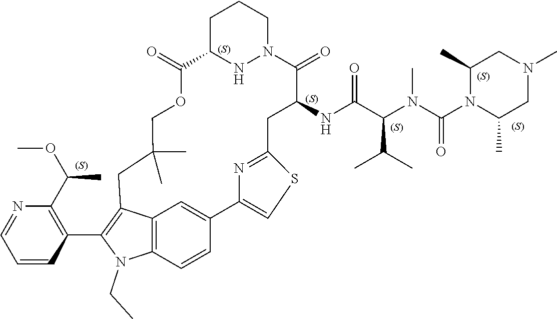 |
| AA610 | 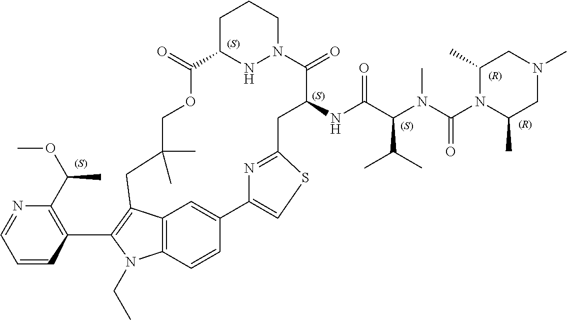 |
| AA611 | 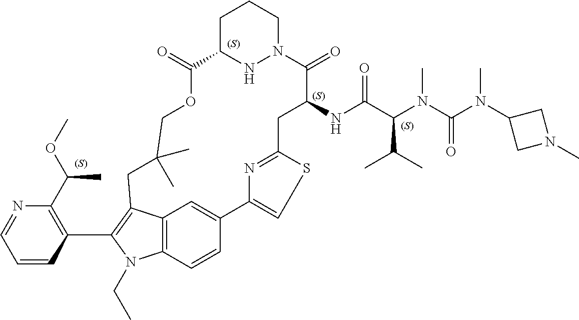 |
| AA612 | 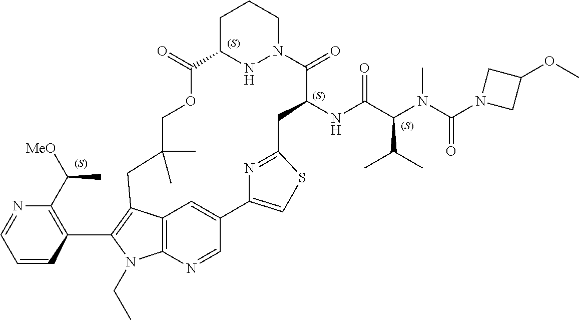 |
| AA613 | 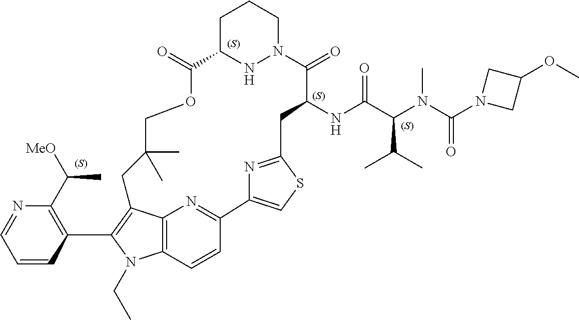 |
| AA614 | 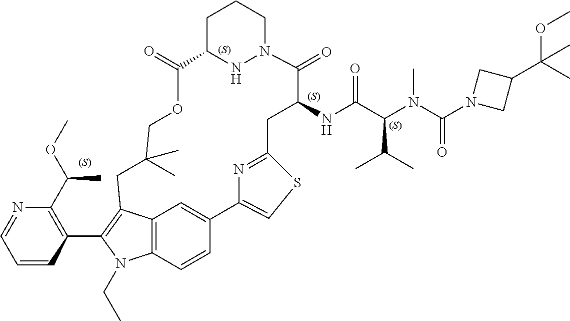 |

Note that some compounds are shown with bonds as flat or wedged. In some instances, the relative stereochemistry of stereoisomers has been determined; in some instances, the absolute stereochemistry has been determined. In some instances, a single Example number corresponds to a mixture of stereoisomers. All stereoisomers of the compounds of the foregoing table are contemplated by the present invention. In particular embodiments, an atropisomer of a compound of the foregoing table is contemplated.
Any compound shown in brackets indicates that the compound is a diastereomer, and the absolute stereochemistry of such diastereomer may not be known.

In some embodiments, a the RAS inhibitor is a RAS(MULTI) inhibitor. In some embodiments, the RAS(MULTI) inhibitor is a compound of Table A2, or a pharmaceutically acceptable salt thereof. In some embodiments, the RAS(MULTI) inhibitor is a compound of Table A2, or a pharmaceutically acceptable salt or atropisomer thereof.

TABLE A2
Certain Compounds of the Present Invention

| Ex # | Structure |
|---|---|
| AB 4 | 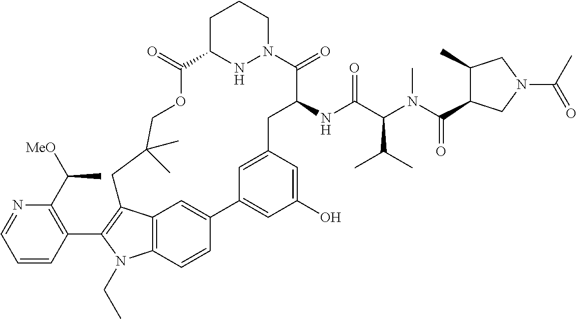 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN(C(C)=O)C[C@@H]1C)C(=O)N1CCC[C@H](N1)C(=O)OCC(C)(C)C2] |
| AB 5 | 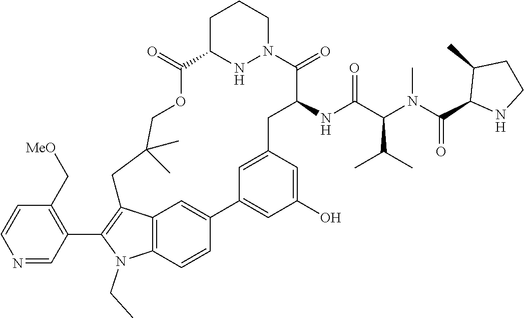 [SMILES: CCn1c(-c2cnccc2COC)c2c3cc(ccc31)-c1cc(O)cc(c1)C[C@H](NC(=O)[C@H](C(C)C)N(C)C(=O)[C@@H]1NCC[C@@H]1C)C(=O)N1CCC[C@H](N1)C(=O)OCC(C)(C)C2] |
| AB 6 | 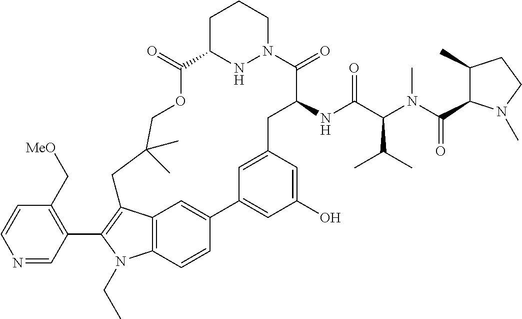 [SMILES: CCn1c(-c2cnccc2COC)c2c3cc(ccc31)-c1cc(O)cc(c1)C[C@H](NC(=O)[C@H](C(C)C)N(C)C(=O)[C@H]1[C@@H](C)CCN1C)C(=O)N1CCC[C@H](N1)C(=O)OCC(C)(C)C2] |
| AB 8 | 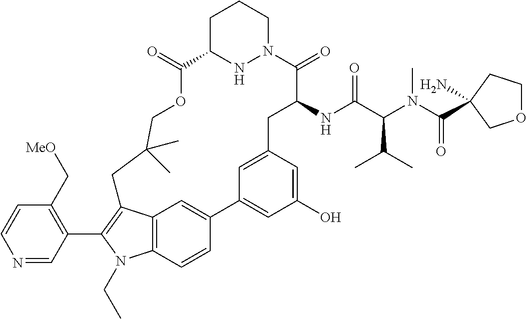 [SMILES: CCn1c(-c2cnccc2COC)c2c3cc(ccc31)-c1cc(O)cc(c1)C[C@H](NC(=O)[C@H](C(C)C)N(C)C(=O)[C@@]1(N)CCOC1)C(=O)N1CCC[C@H](N1)C(=O)OCC(C)(C)C2] |
| AB 9 | 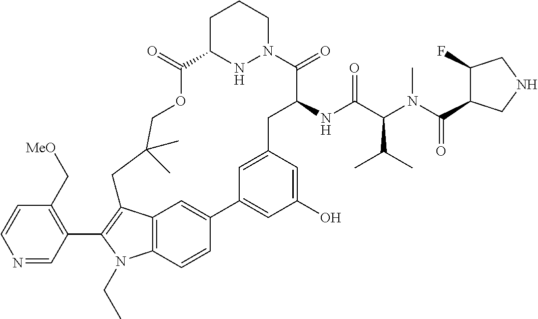 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CNC[C@@H]1F)C(=O)N1CCC[C@H](N1)C(=O)OCC(C)(C)C2] |
| AB 12 | 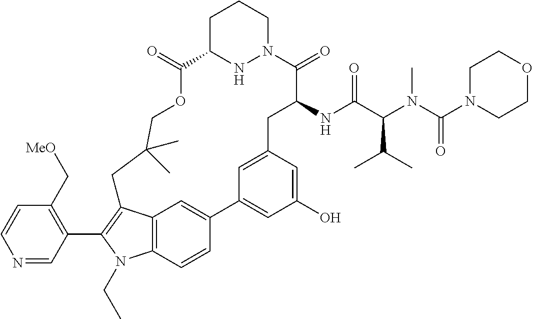 [SMILES: CCn1c(-c2cnccc2COC)c2c3cc(ccc31)-c1cc(O)cc(c1)C[C@H](NC(=O)[C@H](C(C)C)N(C)C(=O)N1CCOCC1)C(=O)N1CCC[C@H](N1)C(=O)OCC(C)(C)C2] |
| AB 13 | 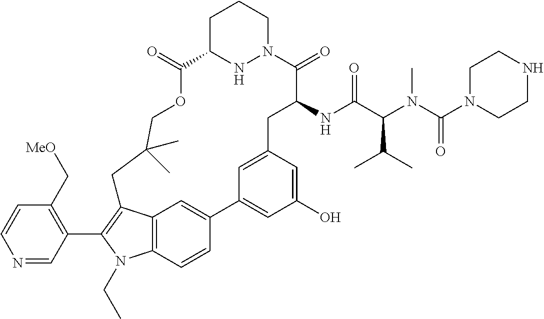 [SMILES: CCn1c(-c2cnccc2COC)c2c3cc(ccc31)-c1cc(O)cc(c1)C[C@H](NC(=O)[C@H](C(C)C)N(C)C(=O)N1CCNCC1)C(=O)N1CCC[C@H](N1)C(=O)OCC(C)(C)C2] |
| AB 19 | 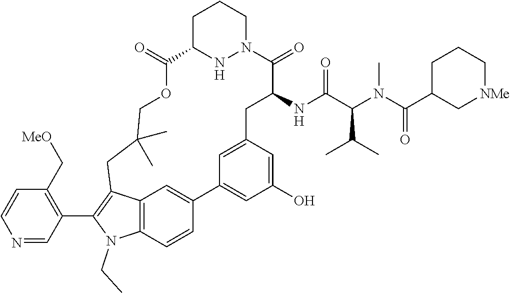 [SMILES: CCn1c(-c2cnccc2COC)c2c3cc(ccc31)-c1cc(O)cc(c1)C[C@H](NC(=O)[C@H](C(C)C)N(C)C(=O)C1CCCN(C)C1)C(=O)N1CCC[C@H](N1)C(=O)OCC(C)(C)C2] |
| AB 44 | 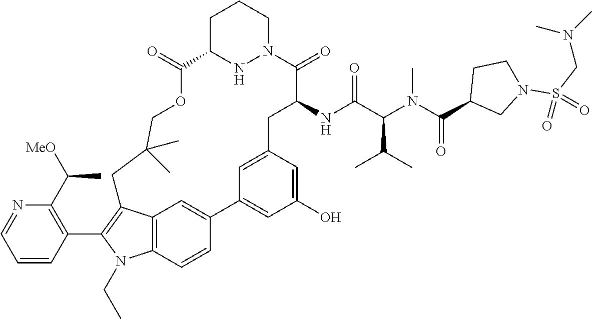 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(S(=O)(=O)CN(C)C)C1)C(=O)N1CCC[C@H](N1)C(=O)OCC(C)(C)C2] |
| AB 47 | 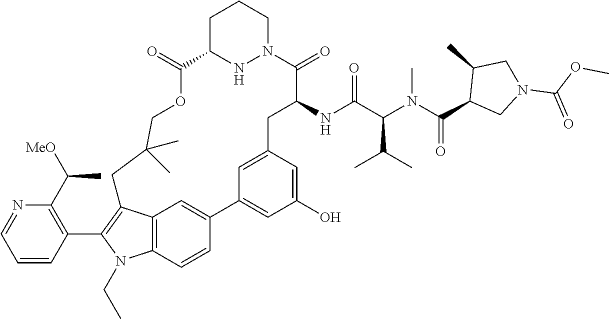 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN(C(=O)OC)C[C@@H]1C)C(=O)N1CCC[C@H](N1)C(=O)OCC(C)(C)C2] |
| AB 57 | 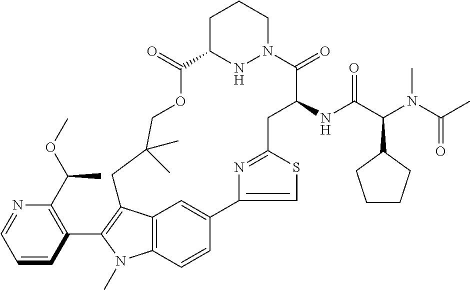 |
| AB 58 | 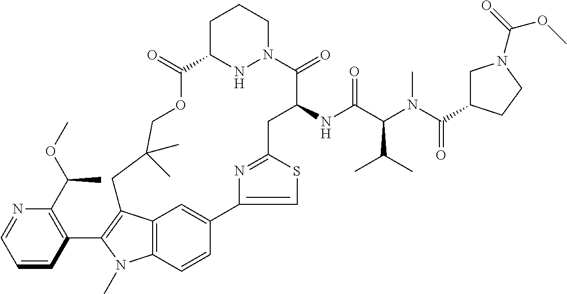 |
| AB 59 | 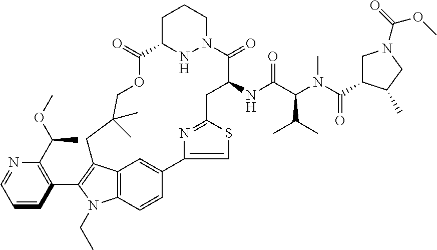 |
| AB 60 | 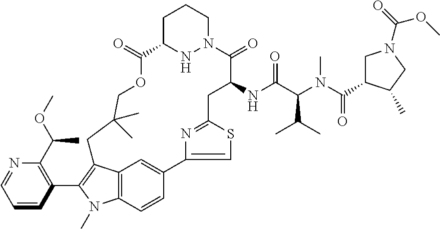 |
| AB 61 | 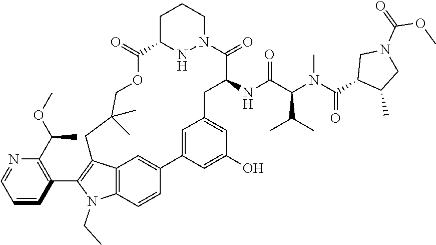 |
| AB 66 | 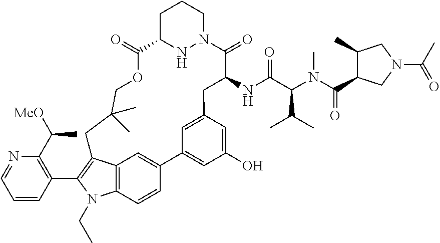 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN(C(C)=O)C[C@@H]1C)C(=O)N1CCC[C@H](N1)C(=O)OCC(C)(C)C2] |
| AB 67 | 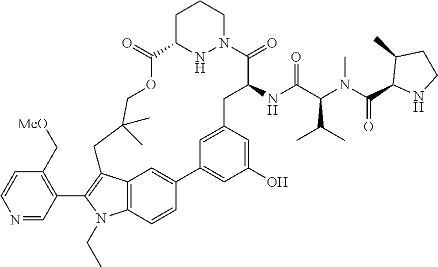 [SMILES: CCn1c(-c2cnccc2COC)c2c3cc(ccc31)-c1cc(O)cc(c1)C[C@H](NC(=O)[C@H](C(C)C)N(C)C(=O)[C@@H]1NCC[C@@H]1C)C(=O)N1CCC[C@H](N1)C(=O)OCC(C)(C)C2] |
| AB 69 | 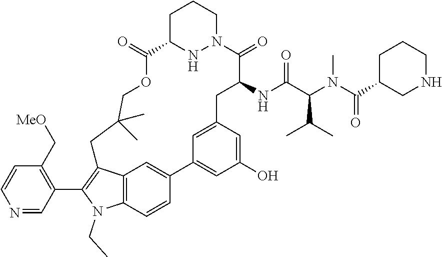 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCNC1)C(=O)N1CCC[C@H](N1)C(=O)OCC(C)(C)C2] |
| AB 71 | 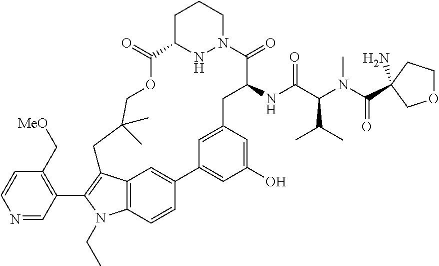 [SMILES: CCn1c(-c2cnccc2COC)c2c3cc(ccc31)-c1cc(O)cc(c1)C[C@H](NC(=O)[C@H](C(C)C)N(C)C(=O)[C@@]1(N)CCOC1)C(=O)N1CCC[C@H](N1)C(=O)OCC(C)(C)C2] |
| AB 73 | 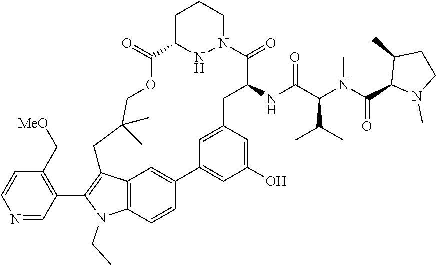 [SMILES: CCn1c(-c2cnccc2COC)c2c3cc(ccc31)-c1cc(O)cc(c1)C[C@H](NC(=O)[C@H](C(C)C)N(C)C(=O)[C@H]1[C@@H](C)CCN1C)C(=O)N1CCC[C@H](N1)C(=O)OCC(C)(C)C2] |
| AB 74 | 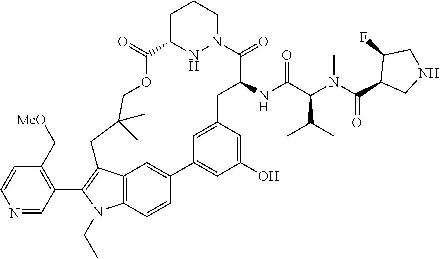 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CNC[C@@H]1F)C(=O)N1CCC[C@H](N1)C(=O)OCC(C)(C)C2] |
| AB 80 | 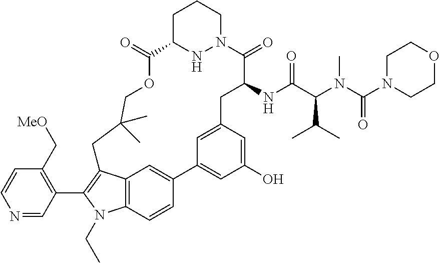 [SMILES: CCn1c(-c2cnccc2COC)c2c3cc(ccc31)-c1cc(O)cc(c1)C[C@H](NC(=O)[C@H](C(C)C)N(C)C(=O)N1CCOCC1)C(=O)N1CCC[C@H](N1)C(=O)OCC(C)(C)C2] |
| AB 81 | 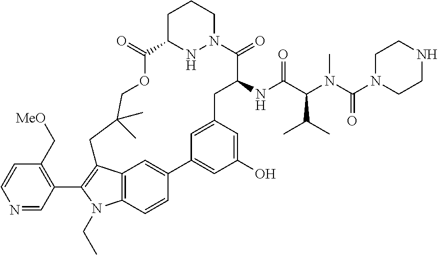 [SMILES: CCn1c(-c2cnccc2COC)c2c3cc(ccc31)-c1cc(O)cc(c1)C[C@H](NC(=O)[C@H](C(C)C)N(C)C(=O)N1CCNCC1)C(=O)N1CCC[C@H](N1)C(=O)OCC(C)(C)C2] |
| AB 94 | 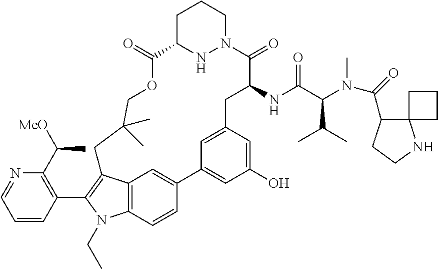 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC13CCC3)C(=O)N1CCC[C@H](N1)C(=O)OCC(C)(C)C2] |
| AB 95 | 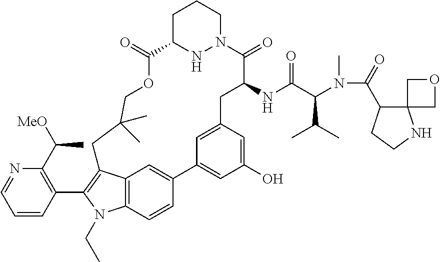 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC13COC3)C(=O)N1CCC[C@H](N1)C(=O)OCC(C)(C)C2] |
| AB 96 | 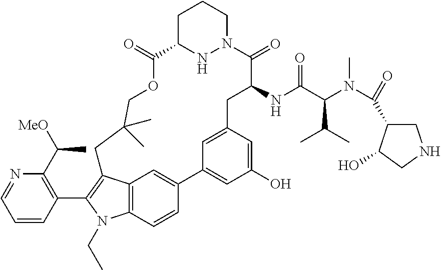 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CNC[C@@H]1O)C(=O)N1CCC[C@H](N1)C(=O)OCC(C)(C)C2] |
| AB 97 | 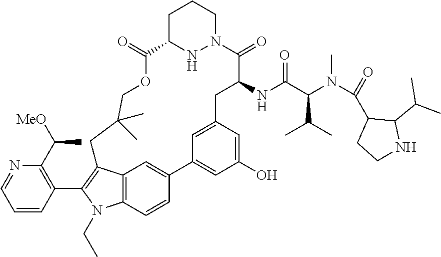 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC1C(C)C)C(=O)N1CCC[C@H](N1)C(=O)OCC(C)(C)C2] |
| AB 99 | 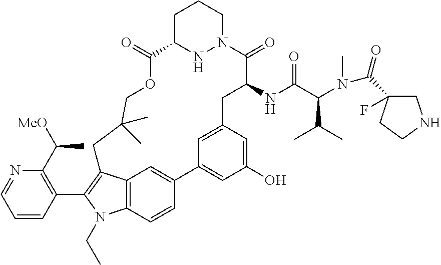 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@]1(F)CCNC1)C(=O)N1CCC[C@H](N1)C(=O)OCC(C)(C)C2] |
| AB 100 | 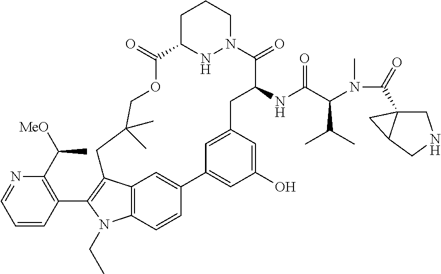 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@]13CNCC1C3)C(=O)N1CCC[C@H](N1)C(=O)OCC(C)(C)C2] |
| AB 104 | 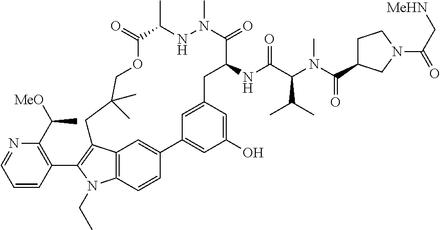 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)CNC)C1)C(=O)N(C)N[C@@H](C)C(=O)OCC(C)(C)C2] |
| AB 106 | 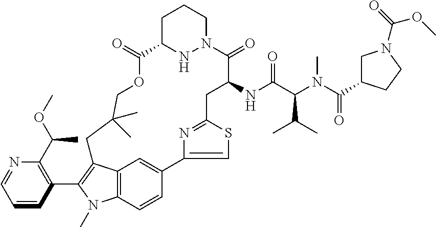 |
| AB 107 | 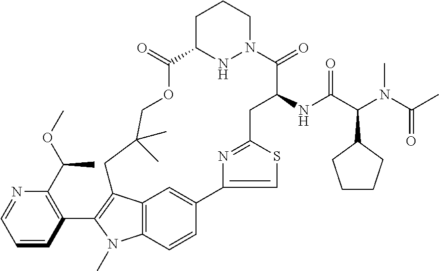 |
| AB 109 | 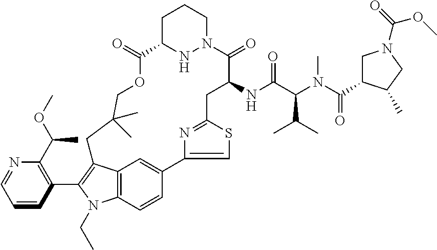 |
| AB 110 | 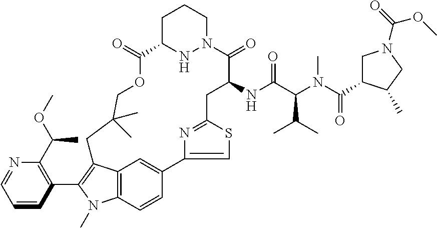 |
| AB 111 | 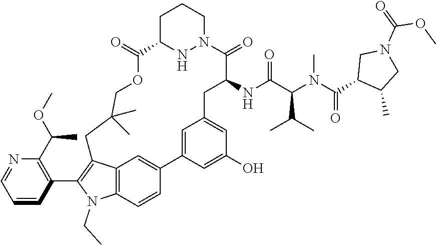 |
| AB 112 | 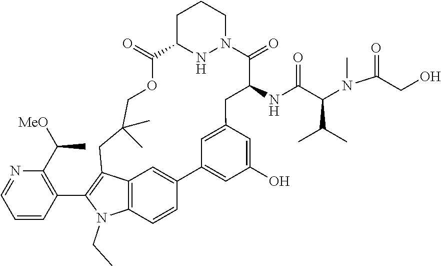 [SMILES: CCn1c(-c2cccnc2[C@H](C)OC)c2c3cc(ccc31)-c1cc(O)cc(c1)C[C@H](NC(=O)[C@H](C(C)C)N(C)C(=O)CO)C(=O)N1CCC[C@H](N1)C(=O)OCC(C)(C)C2] |
| AB 113 | 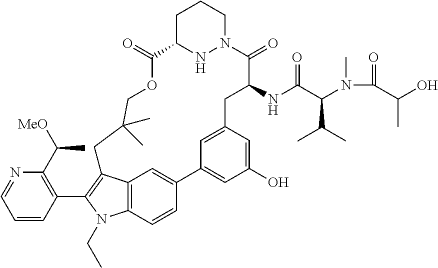 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C(C)O)C(=O)N1CCC[C@H](N1)C(=O)OCC(C)(C)C2] |
| AB 114 | 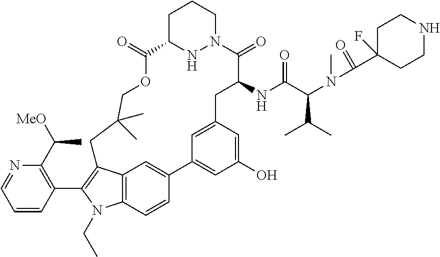 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1(F)CCNCC1)C(=O)N1CCC[C@H](N1)C(=O)OCC(C)(C)C2] |
| AB 117 | 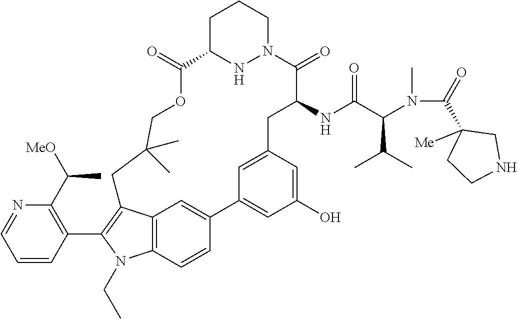 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@]1(C)CCNC1)C(=O)N1CCC[C@H](N1)C(=O)OCC(C)(C)C2] |
| AB 119 | 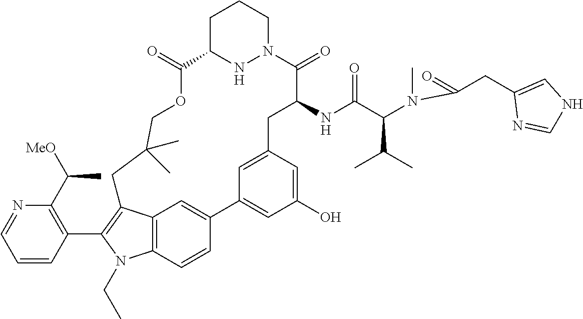 [SMILES: CCn1c(-c2cccnc2[C@H](C)OC)c2c3cc(ccc31)-c1cc(O)cc(c1)C[C@H](NC(=O)[C@H](C(C)C)N(C)C(=O)Cc1c[nH]cn1)C(=O)N1CCC[C@H](N1)C(=O)OCC(C)(C)C2] |
| AB 122 | 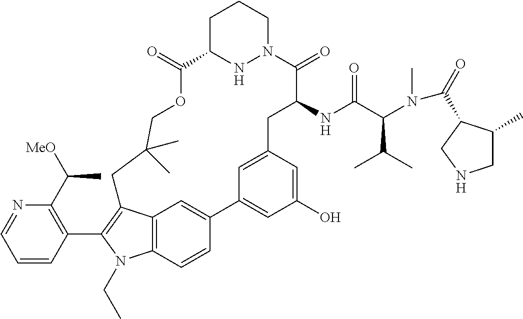 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CNC[C@H]1C)C(=O)N1CCC[C@H](N1)C(=O)OCC(C)(C)C2] |
| AB 123 | 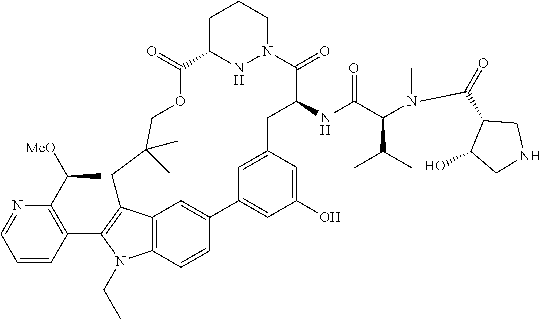 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CNC[C@@H]1O)C(=O)N1CCC[C@H](N1)C(=O)OCC(C)(C)C2] |
| AB 124 | 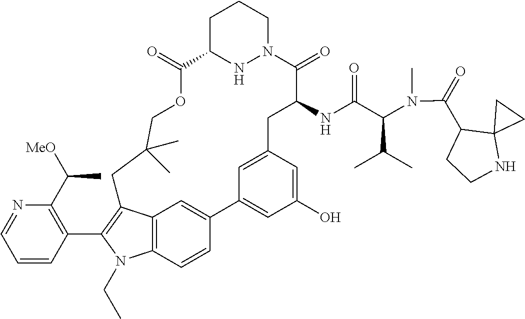 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC13CC3)C(=O)N1CCC[C@H](N1)C(=O)OCC(C)(C)C2] |
| AB 126 | 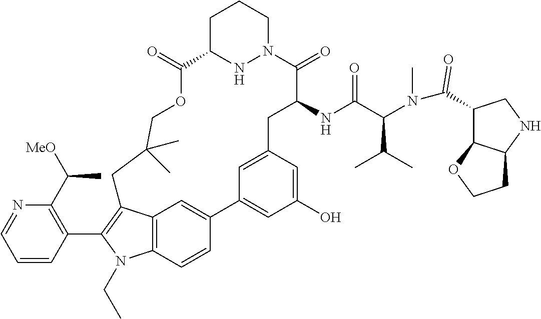 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN[C@H]3CCO[C@H]31)C(=O)N1CCC[C@H](N1)C(=O)OCC(C)(C)C2] |
| AB 128 | 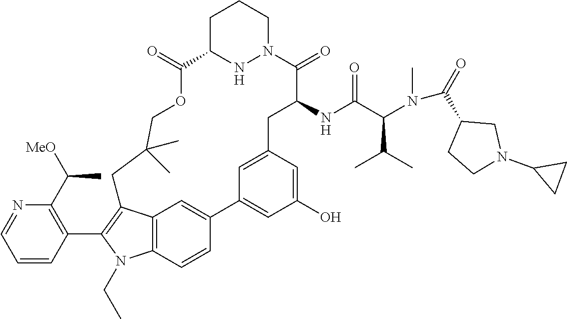 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C3CC3)C1)C(=O)N1CCC[C@H](N1)C(=O)OCC(C)(C)C2] |
| AB 129 | 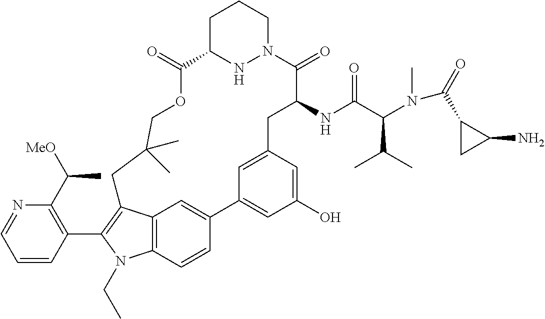 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1C[C@@H]1N)C(=O)N1CCC[C@H](N1)C(=O)OCC(C)(C)C2] |
| AB 130 | 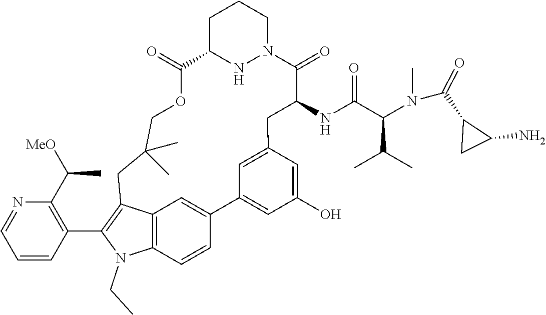 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1C[C@H]1N)C(=O)N1CCC[C@H](N1)C(=O)OCC(C)(C)C2] |
| AB 133 | 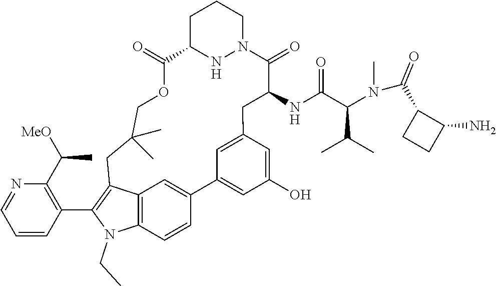 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CC[C@H]1N)C(=O)N1CCC[C@H](N1)C(=O)OCC(C)(C)C2] |
| AB 134 | 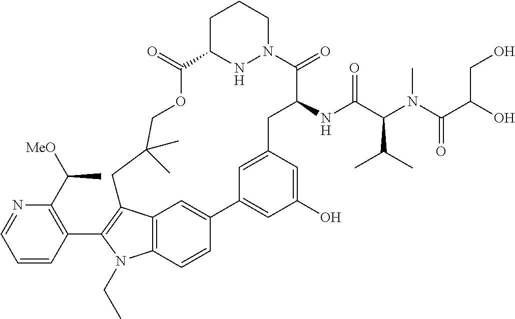 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C(O)CO)C(=O)N1CCC[C@H](N1)C(=O)OCC(C)(C)C2] |
| AB 135 | 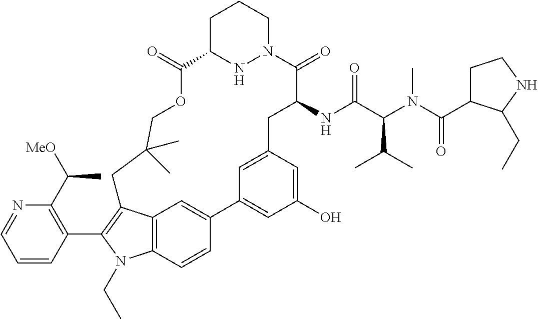 [SMILES: CCC1NCCC1C(=O)N(C)[C@H](C(=O)N[C@H]1Cc2cc(O)cc(c2)-c2ccc3c(c2)c(c(-c2cccnc2[C@H](C)OC)n3CC)CC(C)(C)COC(=O)[C@@H]2CCCN(N2)C1=O)C(C)C] |
| AB 137 | 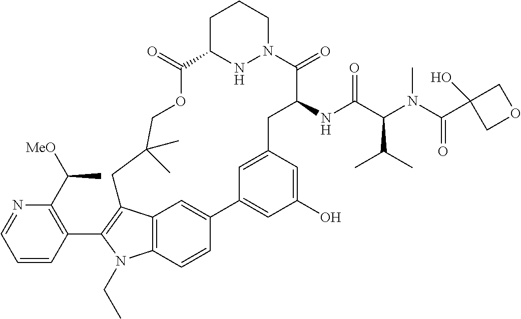 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1(O)COC1)C(=O)N1CCC[C@H](N1)C(=O)OCC(C)(C)C2] |
| AB 138 | 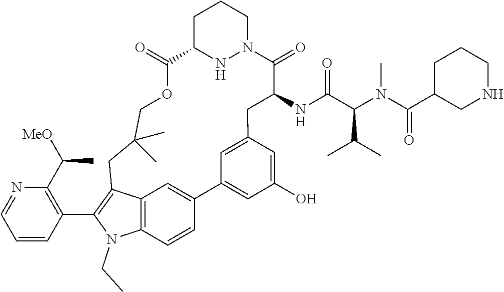 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCCNC1)C(=O)N1CCC[C@H](N1)C(=O)OCC(C)(C)C2] |
| AB 139 | 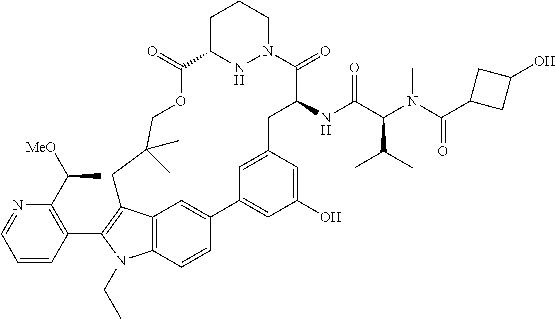 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CC(O)C1)C(=O)N1CCC[C@H](N1)C(=O)OCC(C)(C)C2] |
| AB 141 | 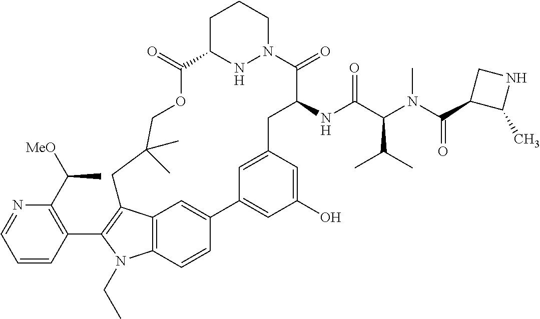 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CN[C@@H]1C)C(=O)N1CCC[C@H](N1)C(=O)OCC(C)(C)C2] |
| AB 143 | 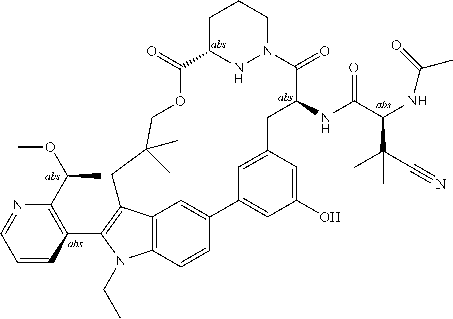 |
| AB 144 | 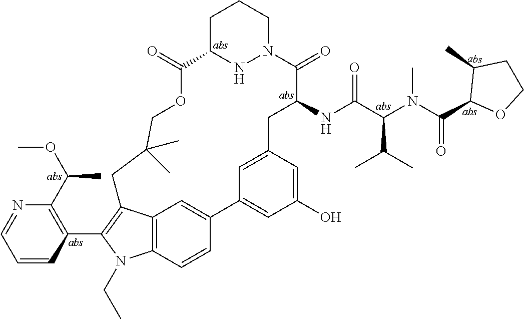 |
| AB 145 | 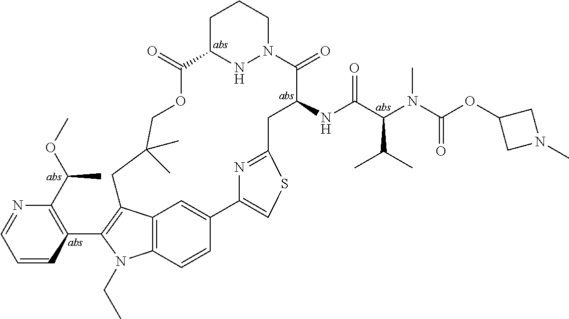 |
| AB 146 | 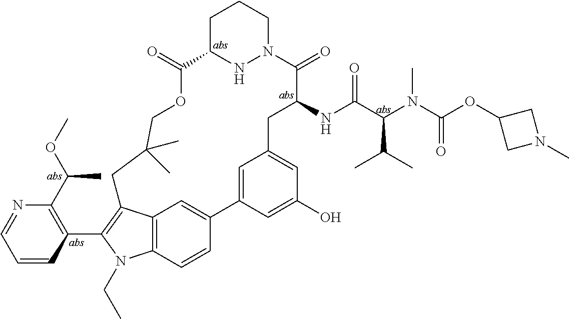 |
| AB 147 | 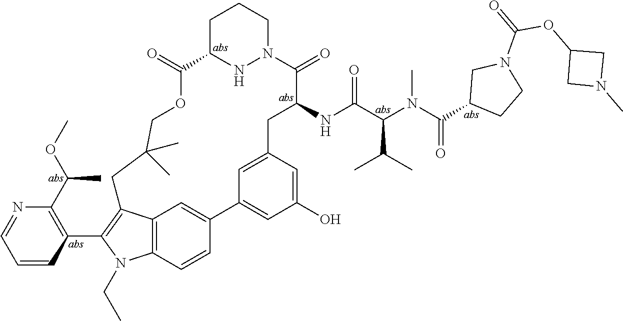 |
| AB 148 | 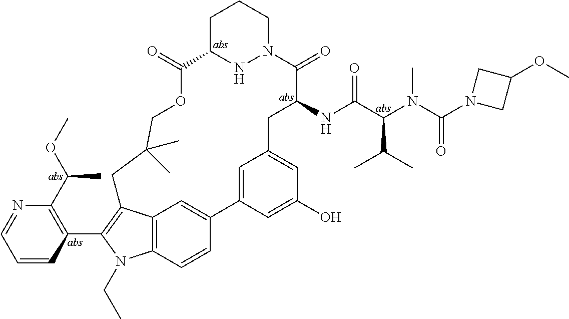 |
| AB 149 | 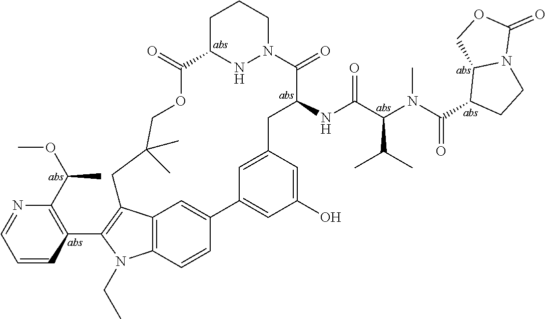 |
| AB 150 | 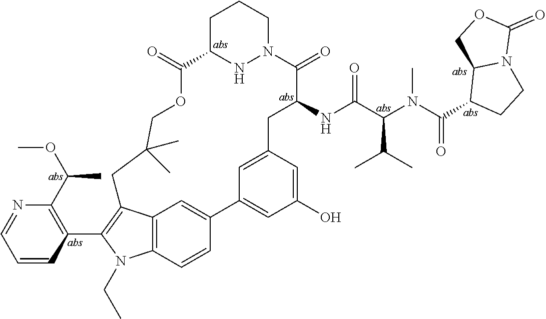 |
| AB 151 | 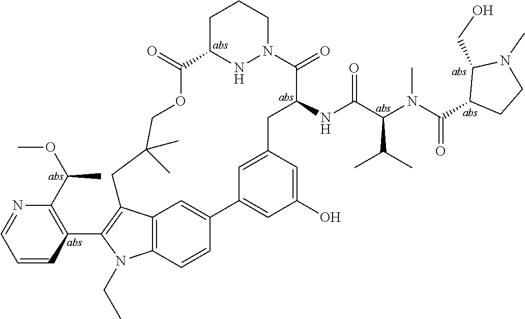 |
| AB 152 | 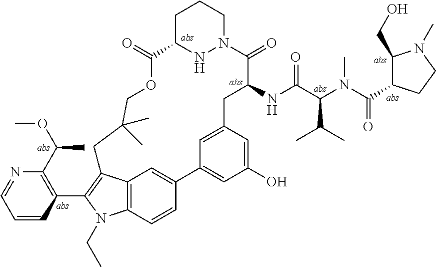 |
| AB 153 | 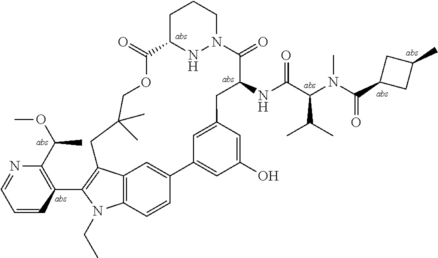 |
| AB 154 | 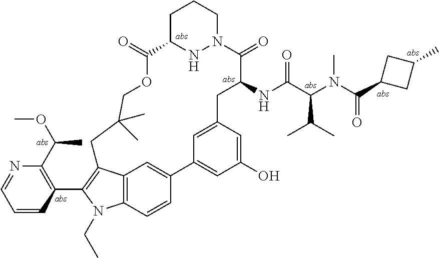 |
| AB 155 | 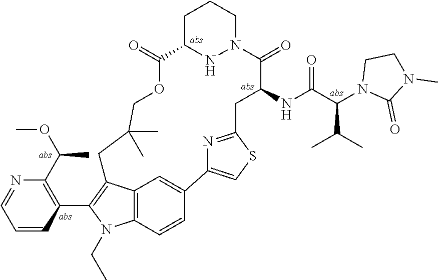 |
| AB 156 | 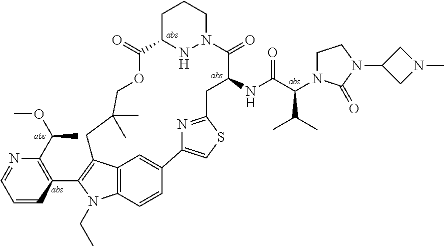 |
| AB 157 | 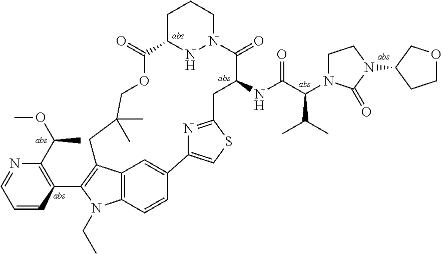 |
| AB 158 | 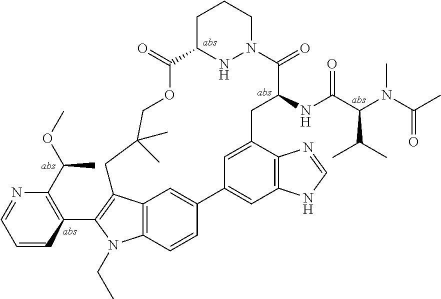 |
| AB 159 | 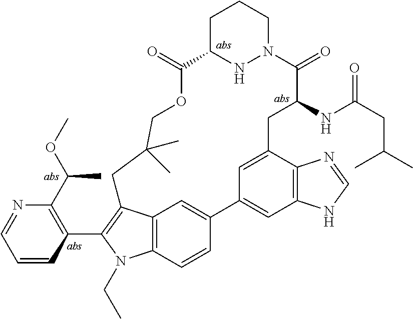 |
| AB 160 | 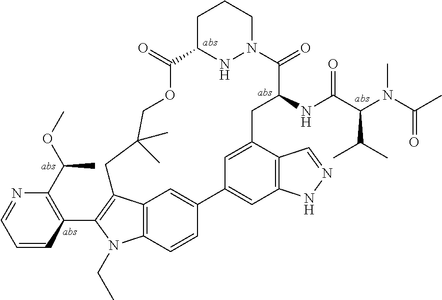 |
| AB 161 | 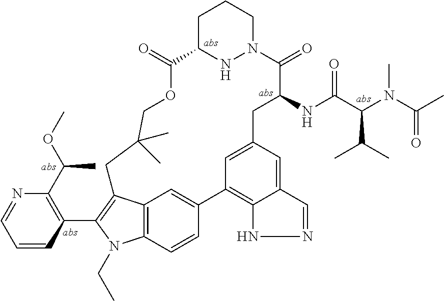 |
| AB 162 | 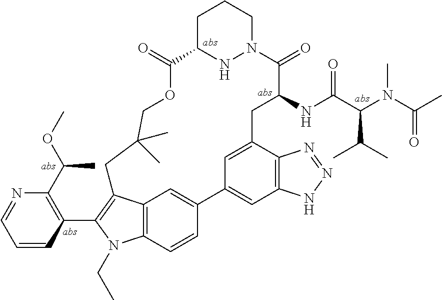 |
| AB 163 | 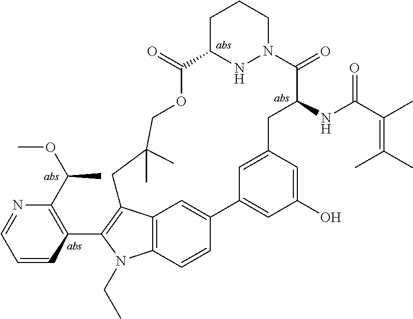 |
| AB 164 | 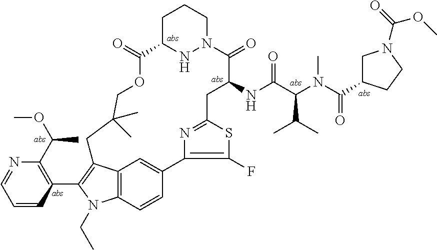 |
| AB 165 | 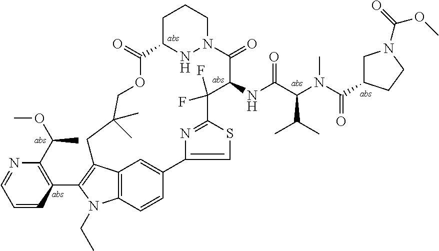 |
| AB 166 | 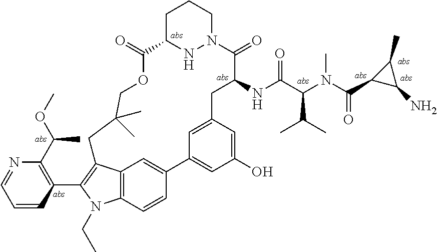 |
| AB 167 | 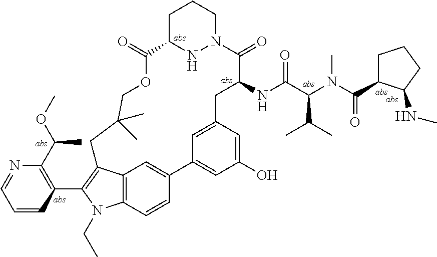 |
| AB 168 | 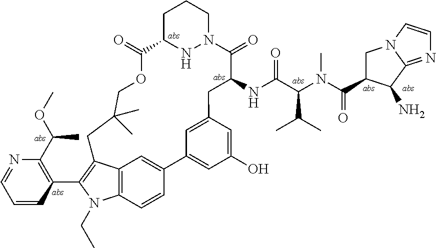 |
| AB 169 | 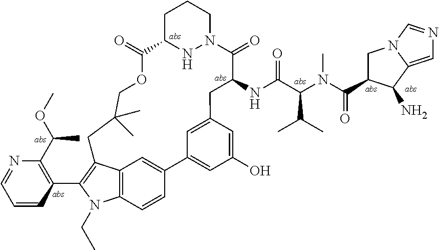 |
| AB 170 | 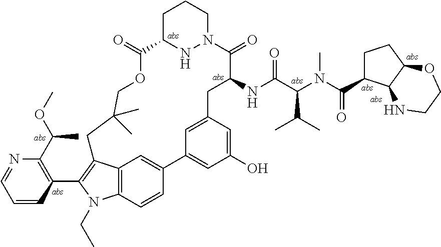 |
| AB 171 | 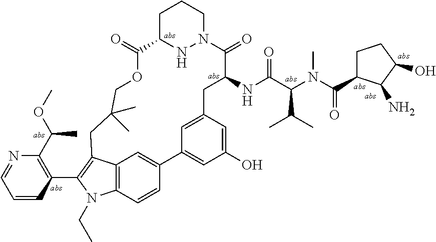 |
| AB 172 | 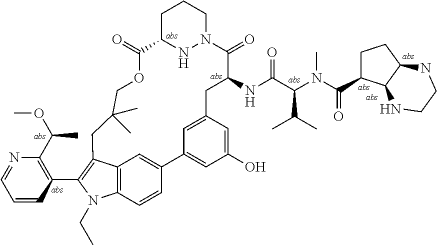 |
| AB 173 | 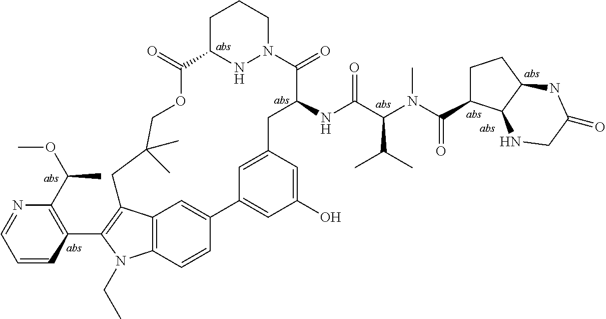 |
| AB 174 | 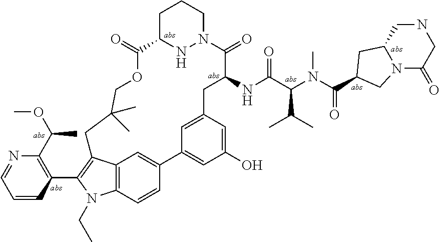 |
| AB 175 | 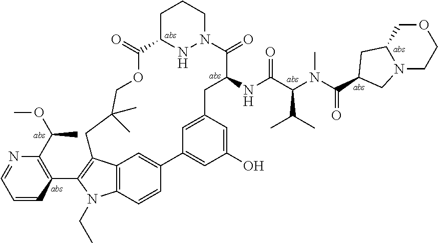 |
| AB 176 | 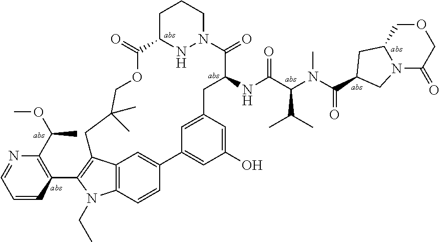 |
| AB 177 | 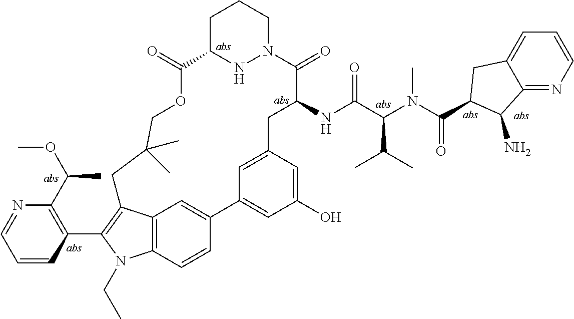 |
| AB 178 | 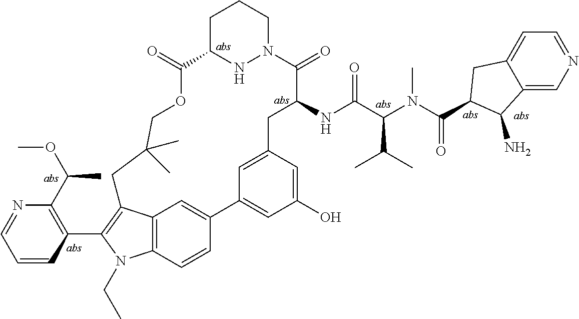 |
| AB 179 | 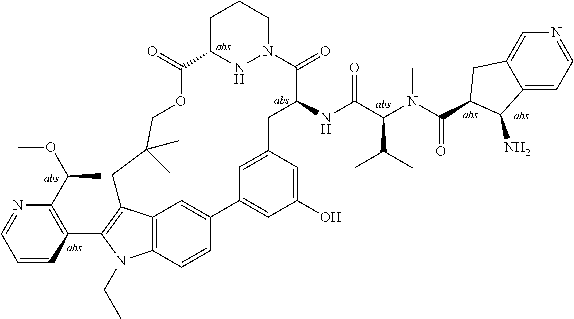 |
| AB 180 | 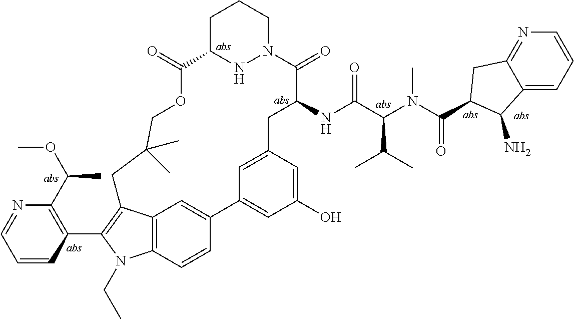 |
| AB 181 | 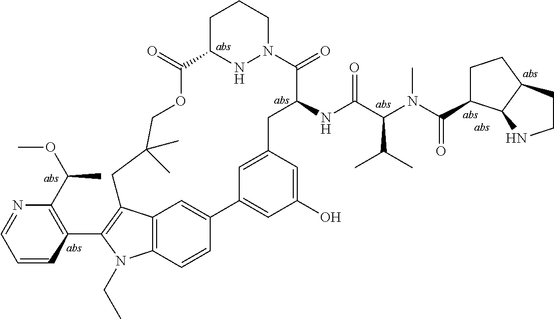 |
| AB 182 | 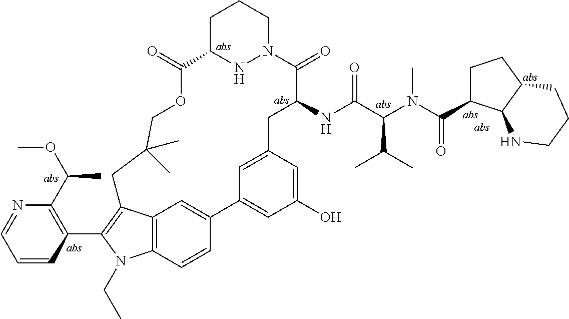 |
| AB 183 | 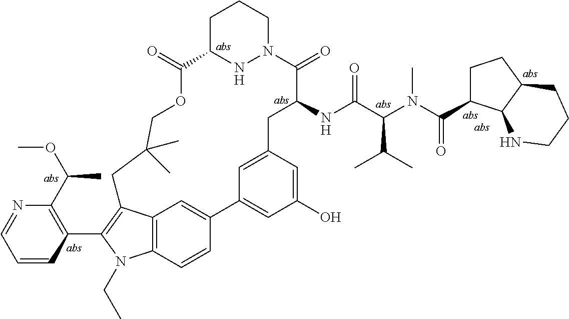 |
| AB 184 | 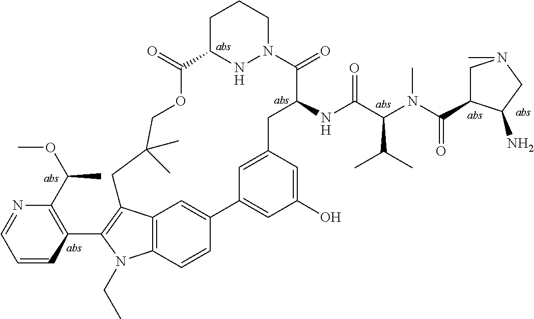 |
| AB 185 | 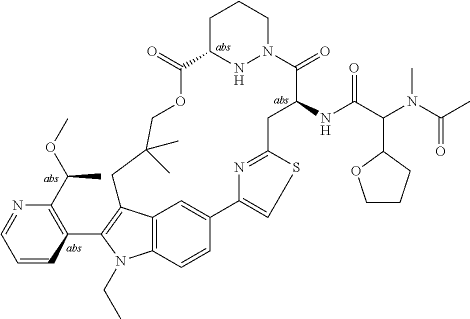 |
| AB 186 | 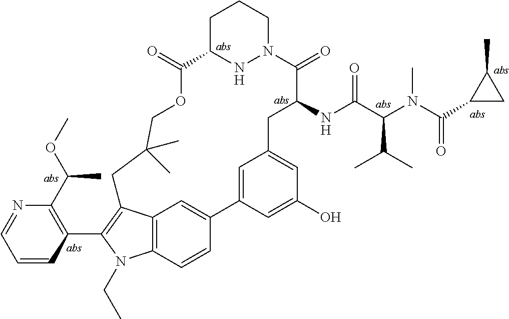 |
| AB 187 | 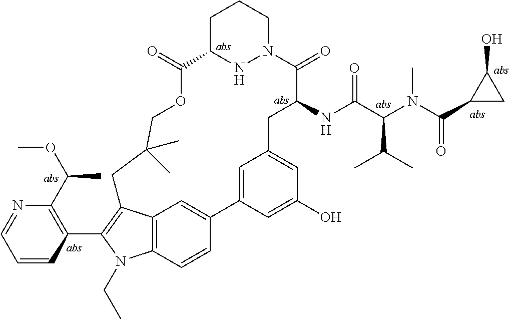 |
| AB 188 | 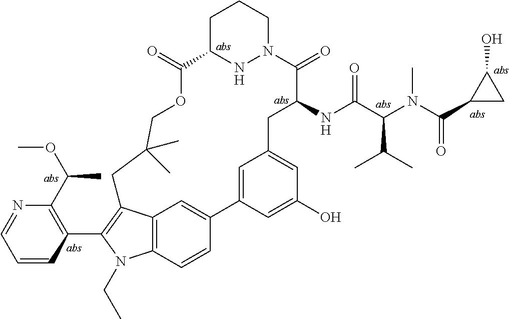 |
| AB 189 | 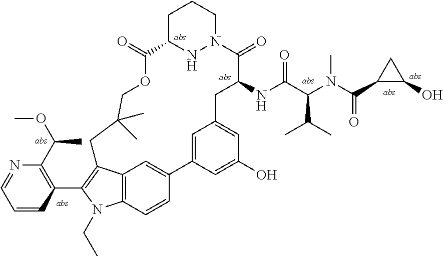 |
| AB 190 | 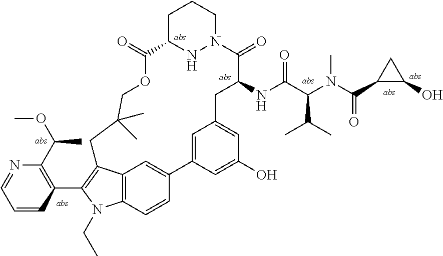 |
| AB 191 | 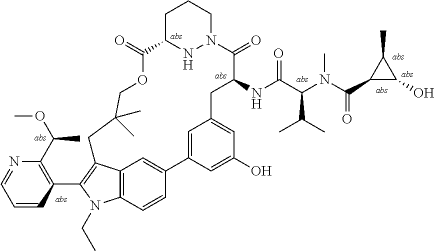 |
| AB 192 | 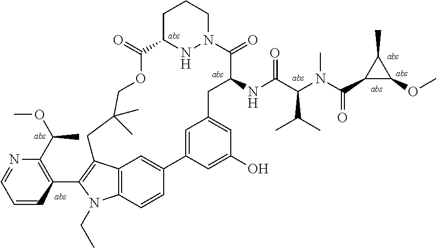 |
| AB 193 | 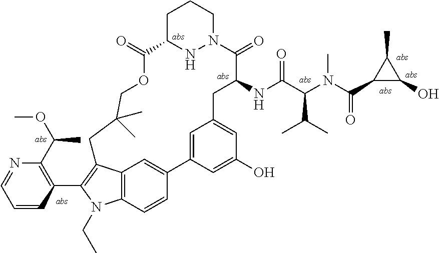 |
| AB 194 | 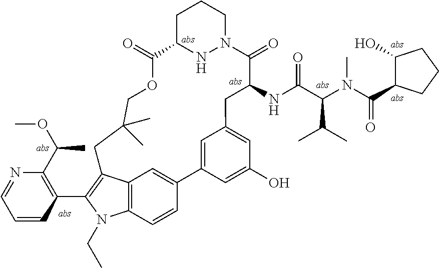 |
| AB 195 | 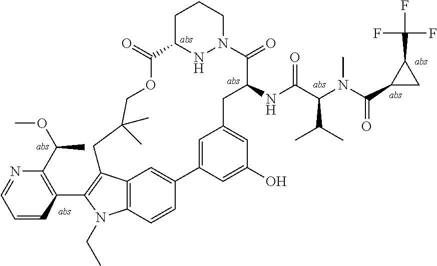 |
| AB 196 | 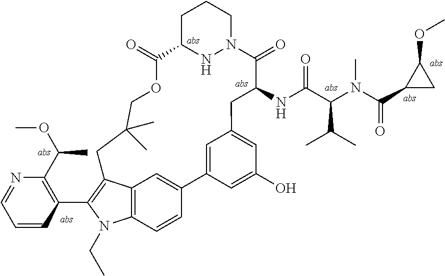 |
| AB 197 | 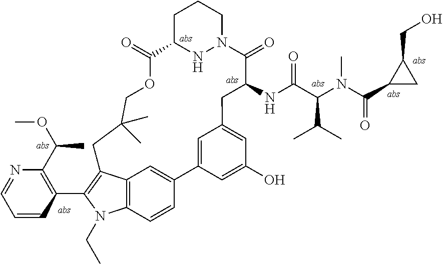 |
| AB 198 | 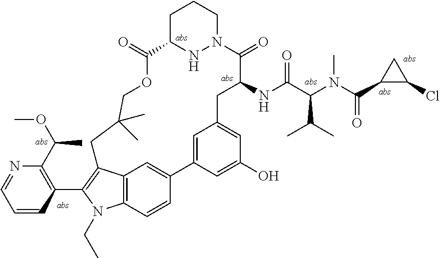 |
| AB 199 | 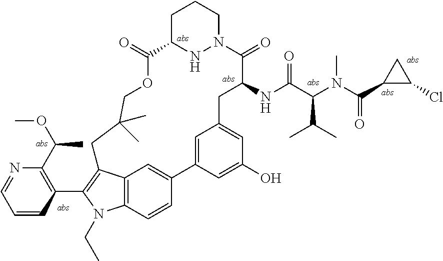 |
| AB 200 | 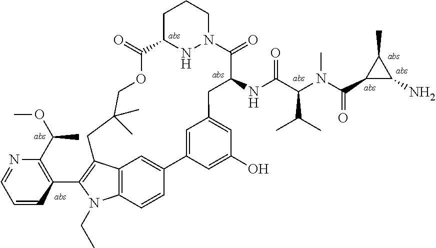 |
| AB 201 | 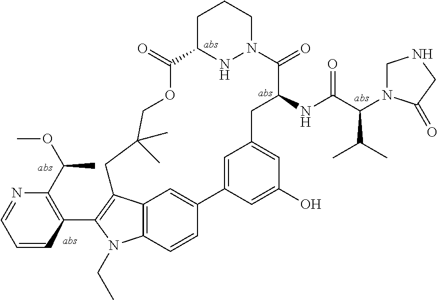 |
| AB 202 | 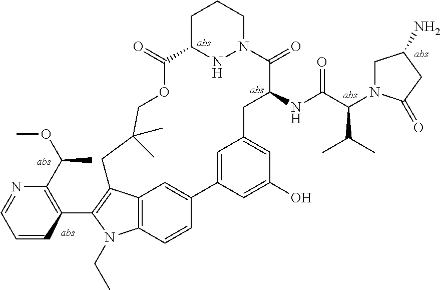 |
| AB 203 | 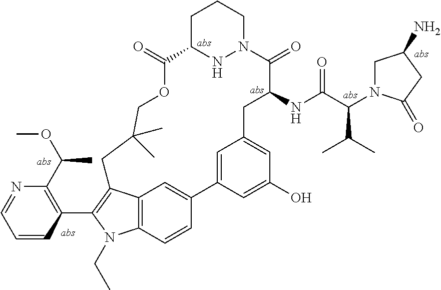 |
| AB 204 | 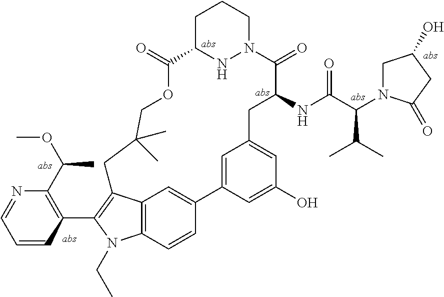 |
| AB 205 | 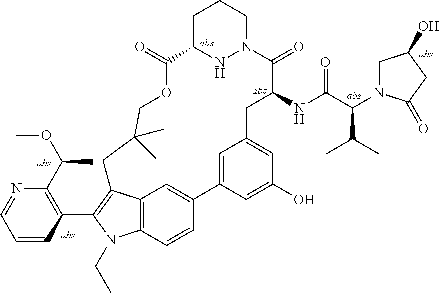 |
| AB 206 | 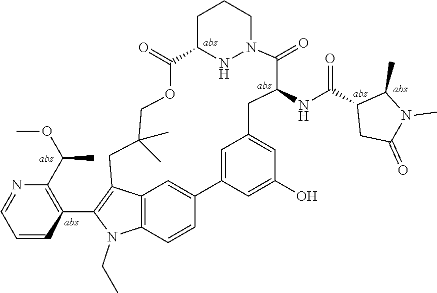 |
| AB 207 | 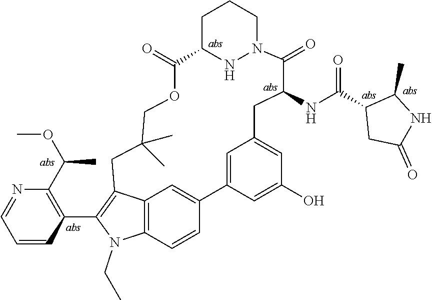 |
| AB 208 | 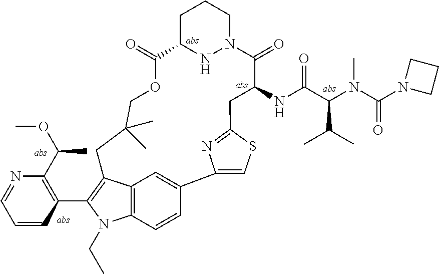 |
| AB 209 | 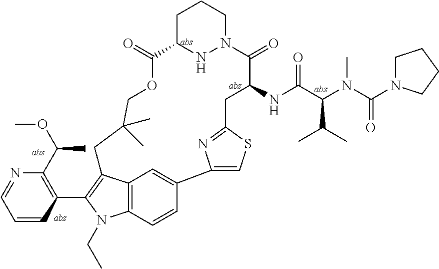 |
| AB 210 | 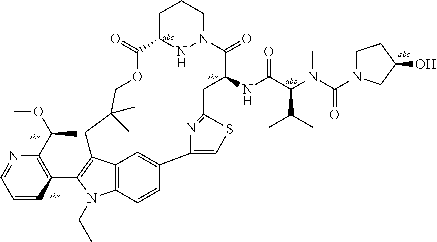 |
| AB 211 | 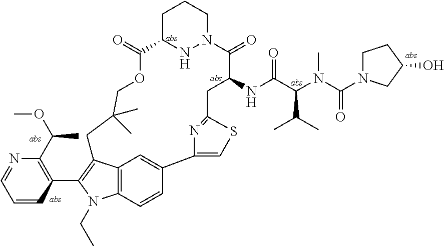 |
| AB 212 | 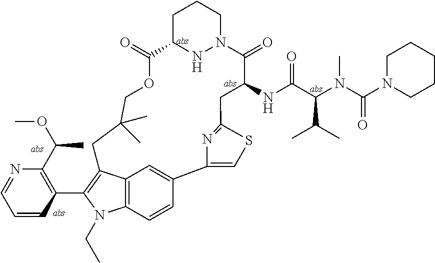 |
| AB 213 | 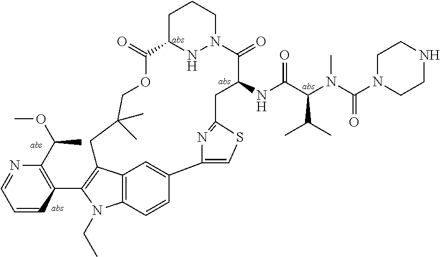 |
| AB 214 | 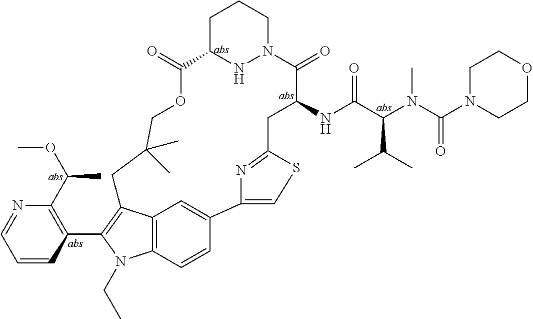 |
| AB 215 | 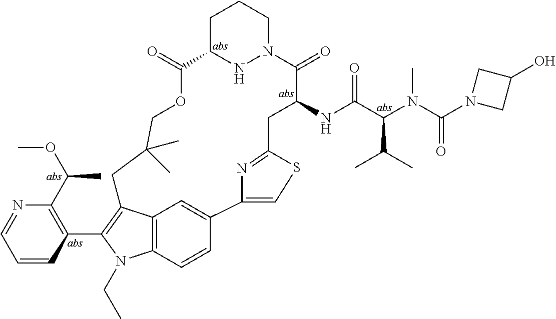 |
| AB 216 | 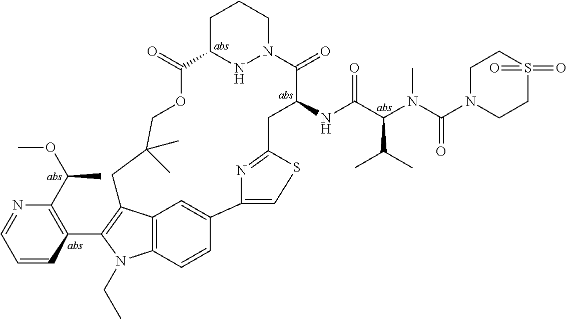 |
| AB 217 | 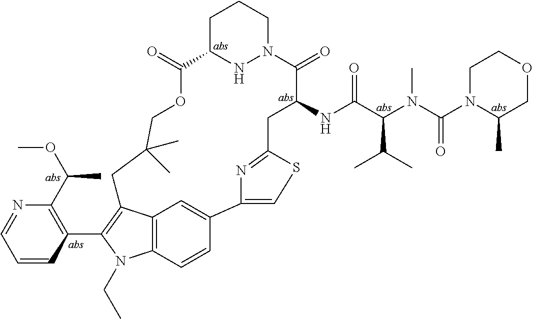 |
| AB 218 | 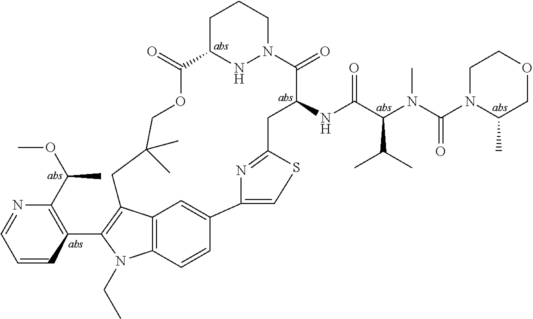 |
| AB 219 | 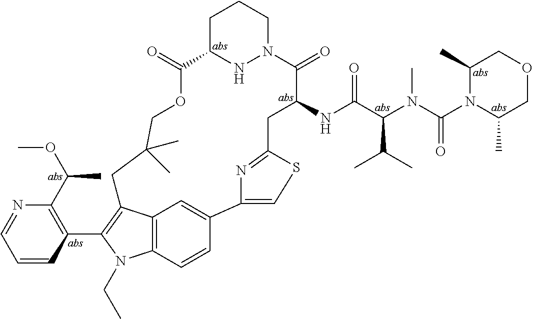 |
| AB 220 | 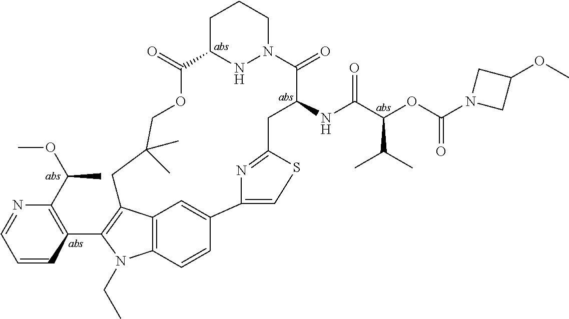 |
| AB 221 | 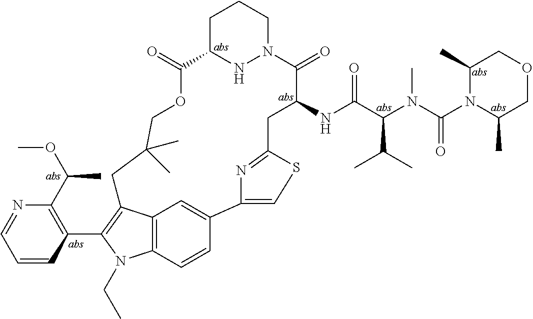 |
| AB 222 | 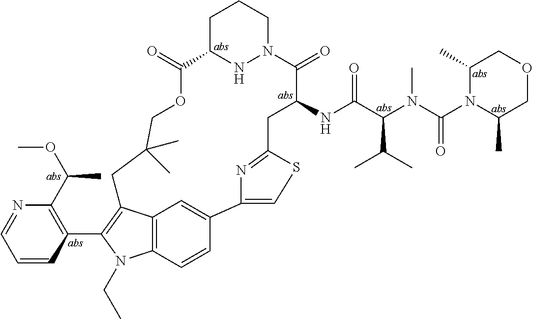 |
| AB 223 | 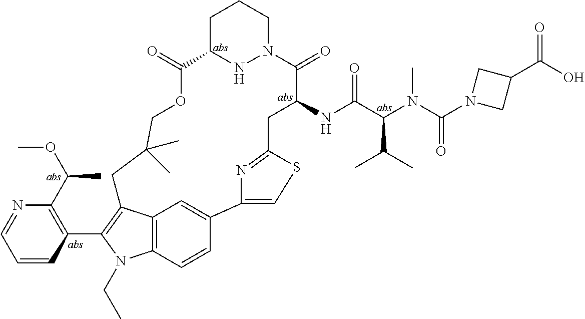 |
| AB 224 | 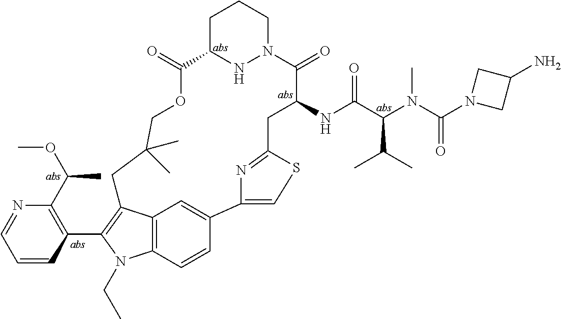 |
| AB 225 | 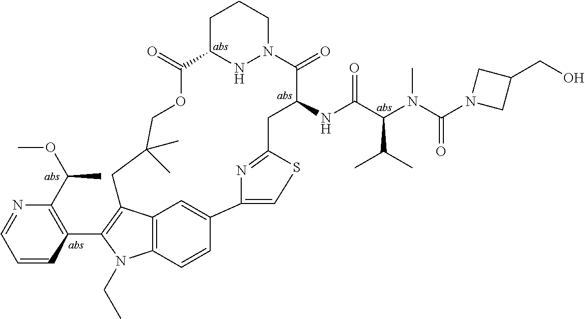 |
| AB 226 | 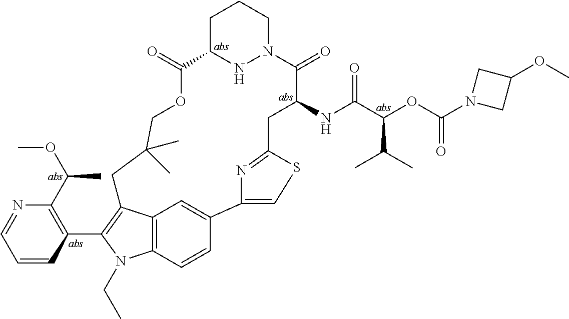 |
| AB 227 | 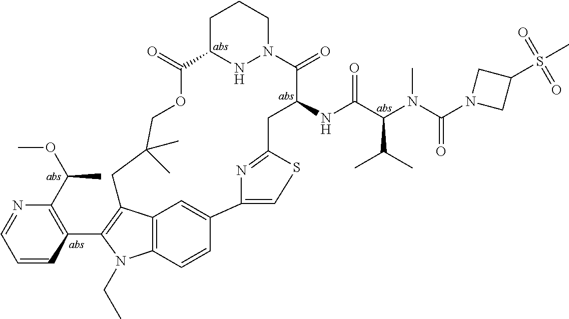 |
| AB 228 | 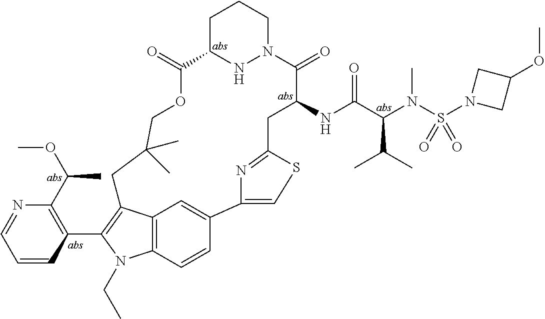 |
| AB 229 | 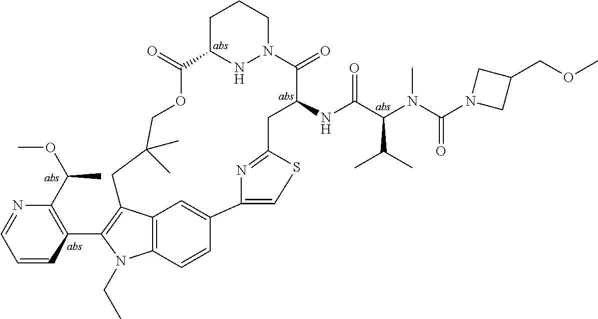 |
| AB 230 | 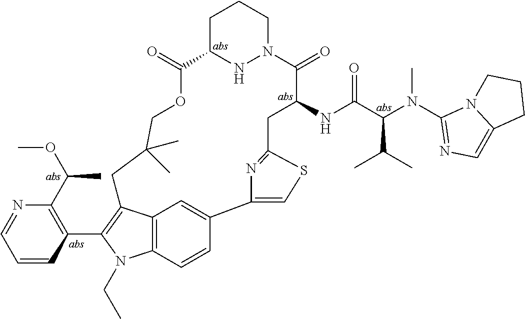 |
| AB 231 | 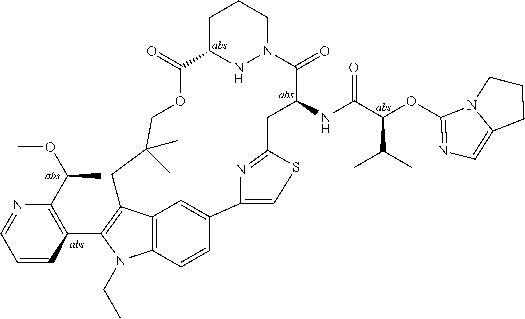 |
| AB 232 | 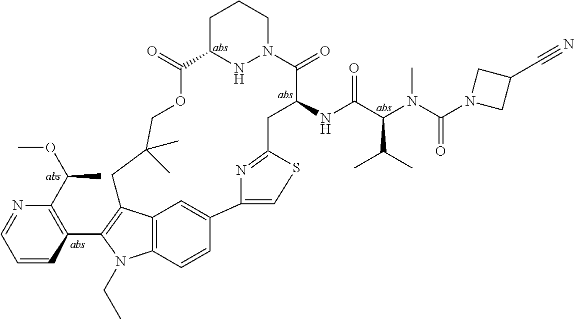 |
| AB 233 | 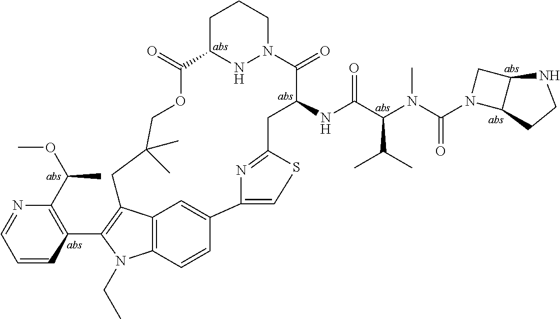 |
| AB 234 | 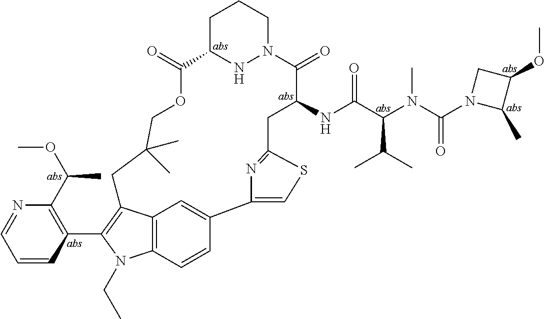 |
| AB 235 | 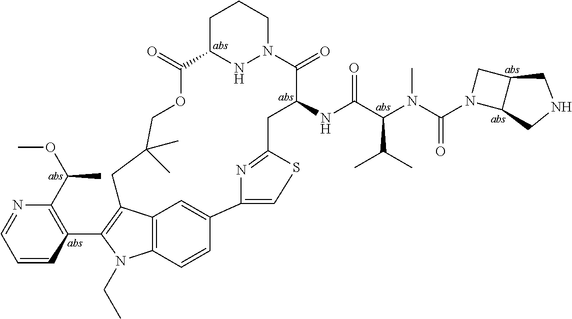 |
| AB 236 | 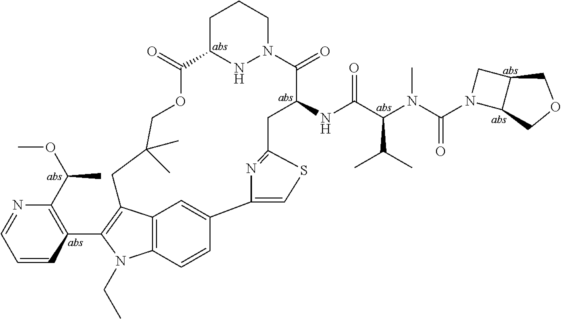 |
| AB 237 | 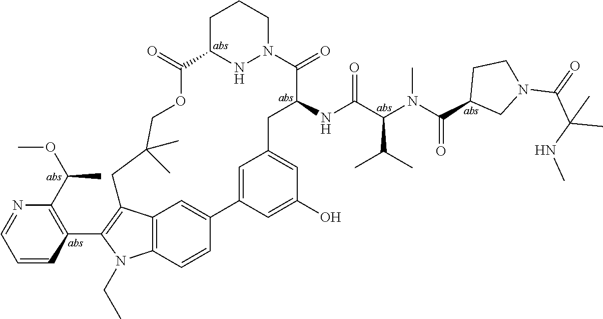 |
| AB 238 | 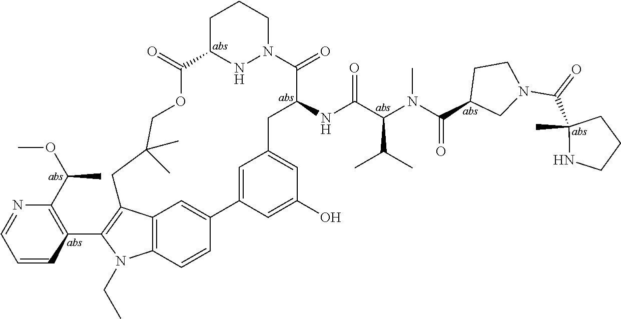 |
| AB 239 | 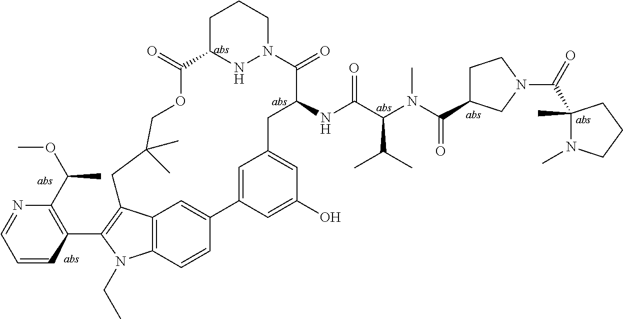 |
| AB 240 | 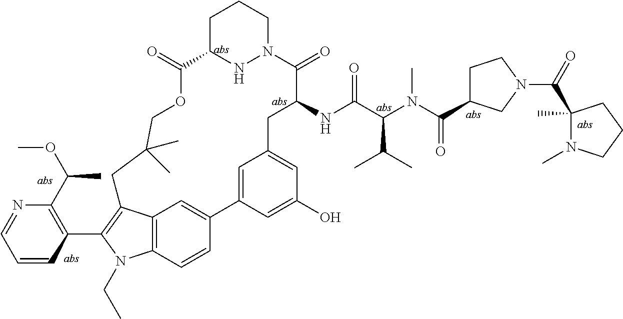 |
| AB 241 | 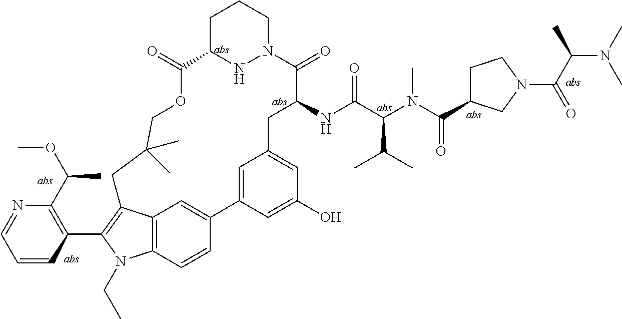 |
| AB 242 | 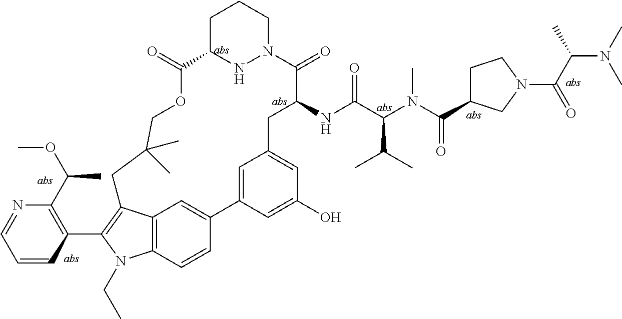 |
| AB 243 | 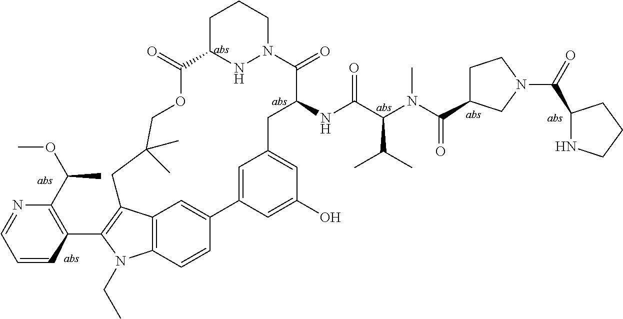 |
| AB 244 | 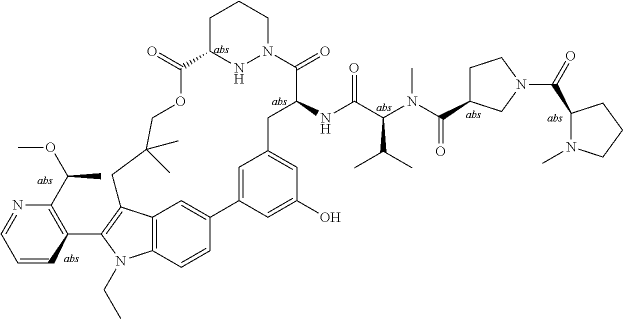 |
| AB 245 | 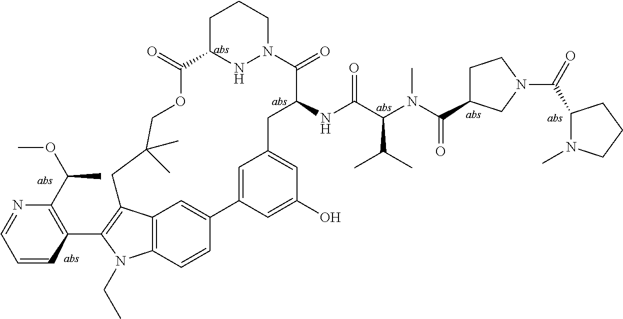 |
| AB 246 | 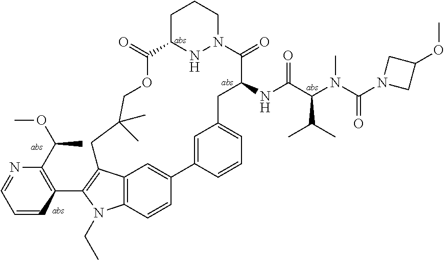 |
| AB 247 | 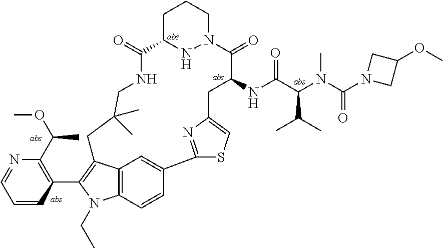 |
| AB 248 | 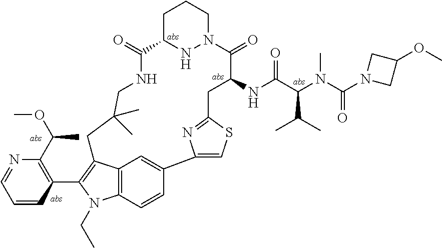 |
| AB 249 | 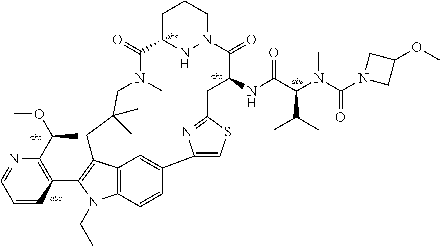 |
| AB 250 | 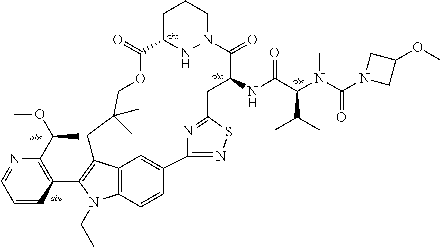 |
| AB 251 | 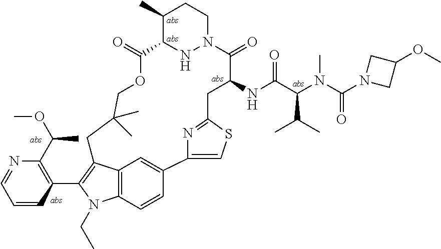 |
| AB 252 | 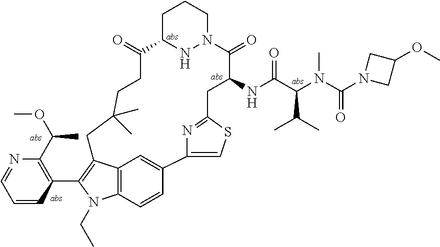 |
| AB 253 | 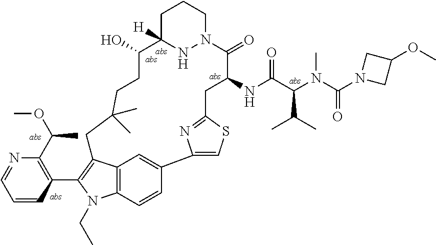 |
| AB 254 | 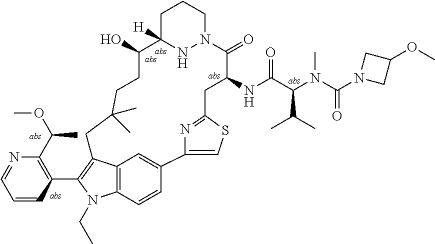 |
| AB 255 | 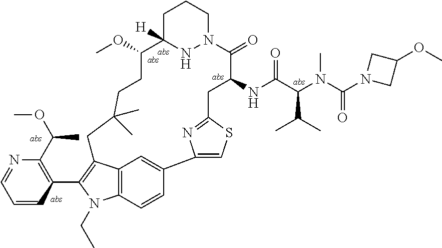 |
| AB 256 | 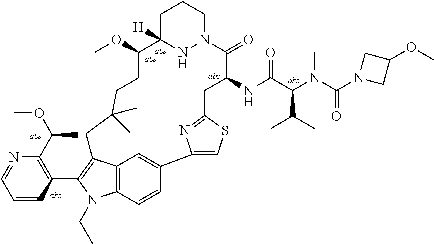 |
| AB 257 | 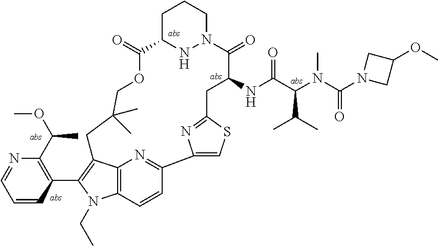 |
| AB 258 | 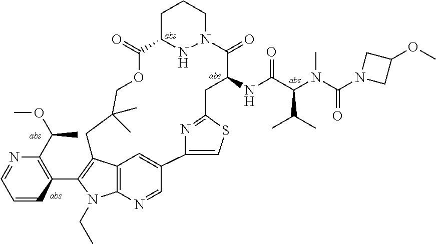 |
| AB 259 | 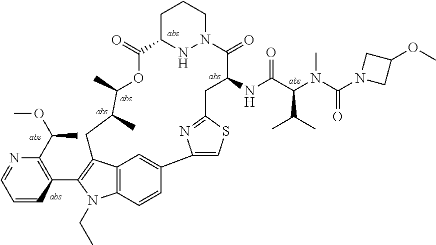 |
| AB 260 | 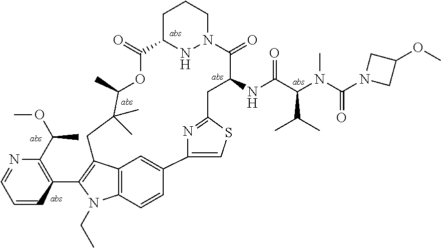 |
| AB 261 | 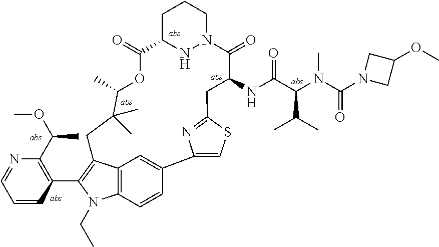 |
| AB 262 | 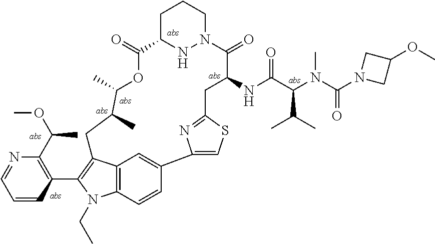 |
| AB 263 | 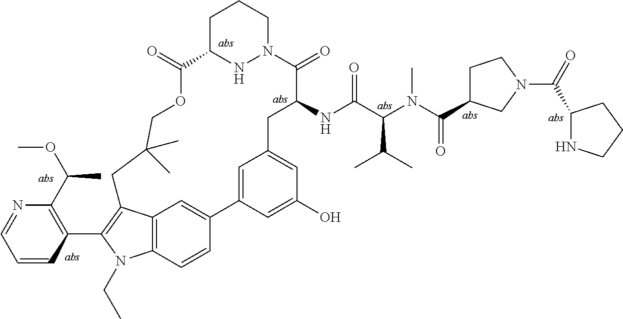 |

Note that some compounds are shown with bonds as flat or wedged. In some instances, the relative
stereochemistry of stereoisomers has been determined; in some instances, the absolute
stereochemistry has been determined. All stereoisomers of the compounds of the foregoing table are
contemplated by the present invention. In particular embodiments, an atropisomer of a compound of
the foregoing table is contemplated.

The compounds described herein may be made from commercially available starting materials or synthesized using known organic, inorganic, or enzymatic processes.

The compounds of the present invention can be prepared in a number of ways well known to those skilled in the art of organic synthesis. By way of example, compounds of table A1 and A2 can be synthesized using the methods described in the Schemes below and in WO 2021/091956, together with synthetic methods known in the art of synthetic organic chemistry, or variations thereon as appreciated by those skilled in the art. These methods include but are not limited to those methods described in the Schemes below or as described in WO 2021/091956, incorporated herein by reference.

Compounds of Table A1 herein were prepared using methods disclosed herein or were prepared using methods disclosed herein combined with the knowledge of one of skill in the art. Compounds of Table A2 may be prepared using methods disclosed herein or may be prepared using methods disclosed herein combined with the knowledge of one of skill in the art.

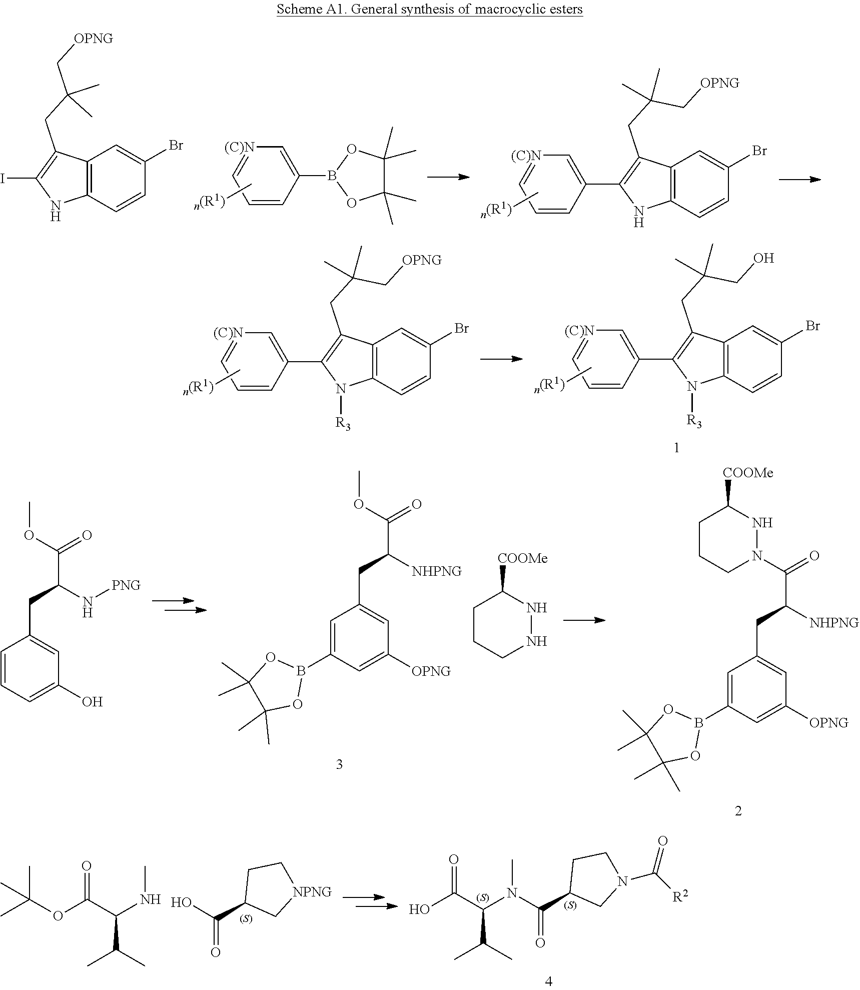

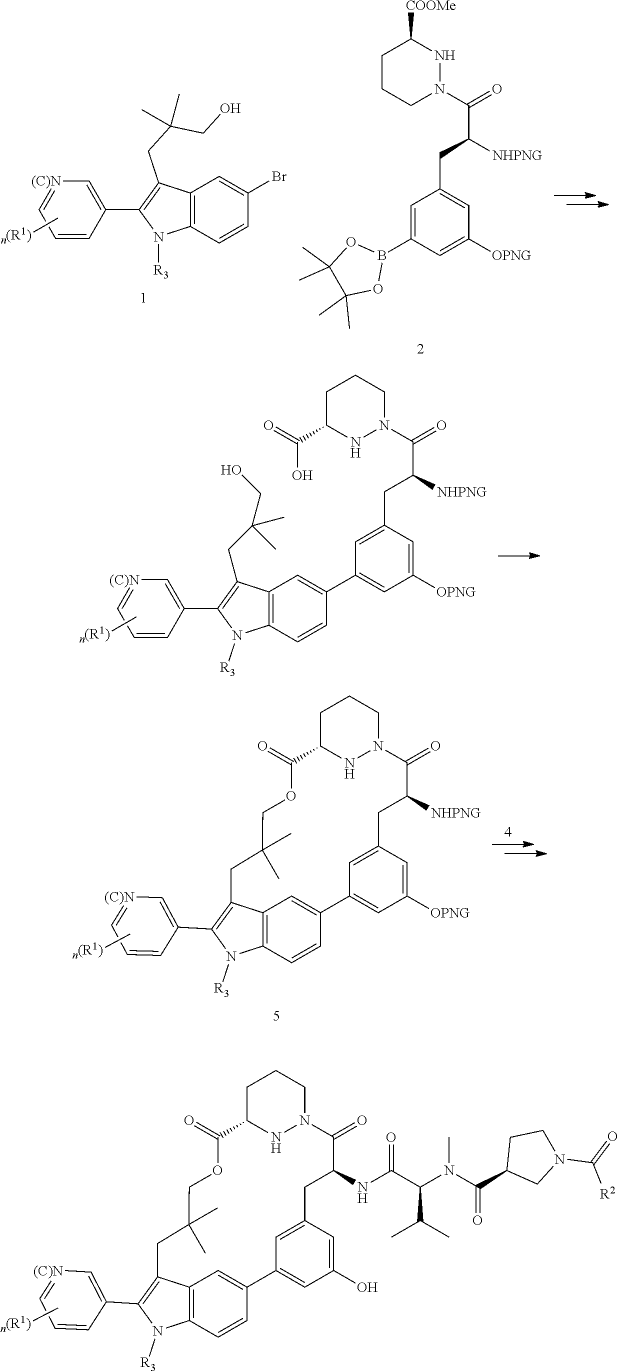

A general synthesis of macrocyclic esters is outlined in Scheme A1. An appropriately substituted Aryl Indole intermediate (1) can be prepared in three steps starting from protected 3-(5-bromo-2-iodo-1H-indol-3-yl)-2,2-dimethylpropan-1-ol and appropriately substituted boronic acid, including Palladium mediated coupling, alkylation, and de-protection reactions. Methyl-amino-hexahydropyridazine-3-carboxylate-boronic ester (2) can be prepared in three steps, including protection, Iridium catalyst mediated borylation, and coupling with methyl (S)-hexahydropyridazine-3-carboxylate.

An appropriately substituted acetylpyrrolidine-3-carbonyl-N-methyl-L-valine (4) can be made by coupling of methyl-L-valinate and protected (S)-pyrrolidine-3-carboxylic acid, followed by deprotection, coupling with an appropriately substituted carboxylic acid, and a hydrolysis step.

The final macrocyclic esters can be made by coupling of methyl-amino-hexahydropyridazine-3-carboxylate-boronic ester (2) and intermediate (1) in the presence of Pd catalyst followed by hydrolysis and macrolactonization steps to result in an appropriately protected macrocyclic intermediate (5). Deprotection and coupling with an appropriately substituted acetylpyrrolidine-3-carbonyl-N-methyl-L-valine (4) results in a macrocyclic product. Additional deprotection or functionalization steps are be required to produce a final compound. For example, a person of skill in the art would be able to install into a macrocyclic ester a desired —B-L-W group of a compound of Formula (AI), where B, L and Ware defined herein, including by using methods exemplified in the Example section herein.

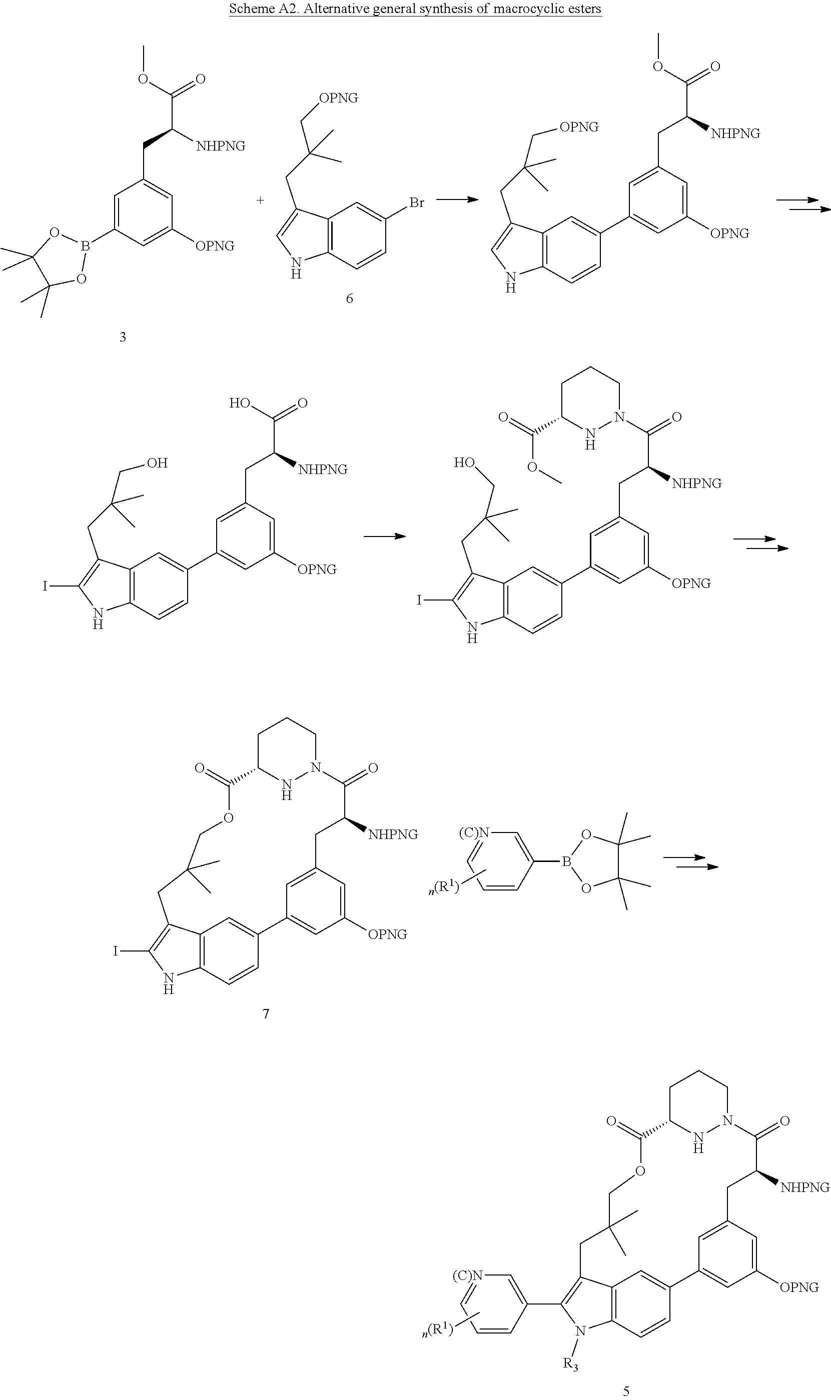

Alternatively, macrocyclic esters can be prepared as described in Scheme 2. An appropriately protected bromo-indolyl (6) can be coupled in the presence of Pd catalyst with boronic ester (3), followed by iodination, deprotection, and ester hydrolysis. Subsequent coupling with methyl (S)-hexahydropyridazine-3-carboxylate, followed by hydrolysis and macrolactonization can result in iodo intermediate (7). Coupling in the presence of Pd catalyst with an appropriately substituted boronic ester and alkylation can yield fully a protected macrocycle (5). Additional deprotection or functionalization steps are required to produce a final compound. For example, a person of skill in the art would be able to install into a macrocyclic ester a desired —B-L-W group of a compound of Formula (AI), where B, L and Ware defined herein, including by using methods exemplified in the Example section herein.

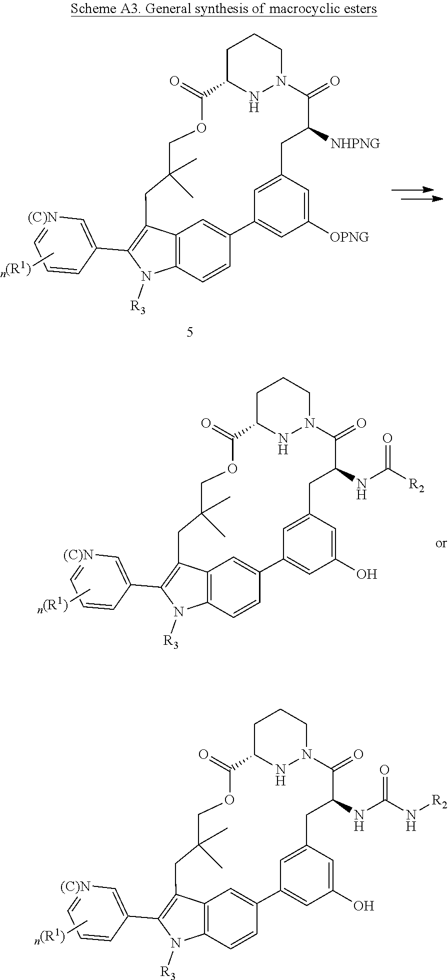

Alternatively, fully a protected macrocycle (5) can be deprotected and coupled with an appropriately substituted coupling partners, and deprotected to results in a macrocyclic product. Additional deprotection or functionalization steps are be required to produce a final compound. For example, a person of skill in the art would be able to install into a macrocyclic ester a desired —B-L-W group of a compound of Formula (AI), where B, L and Ware defined herein, including by using methods exemplified in the Example section herein.

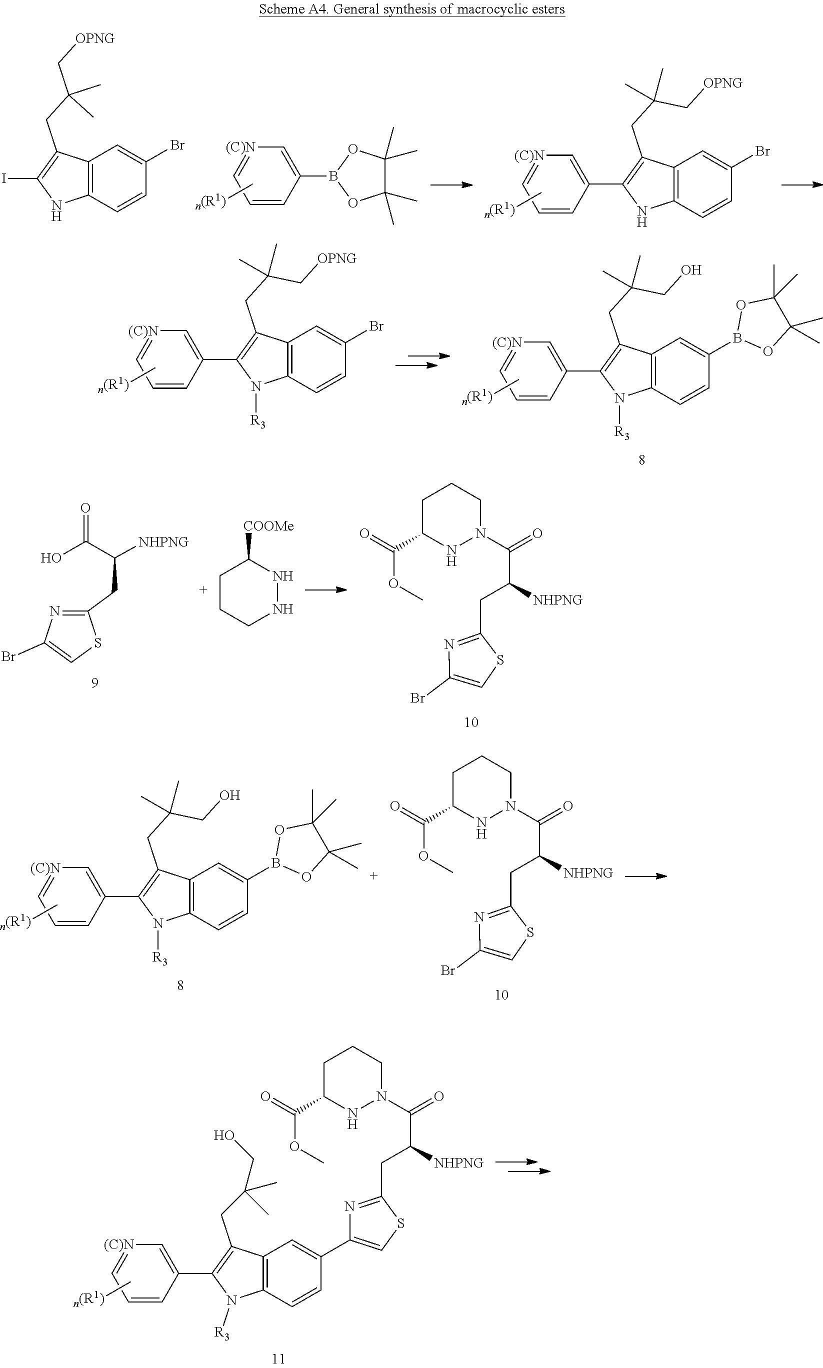

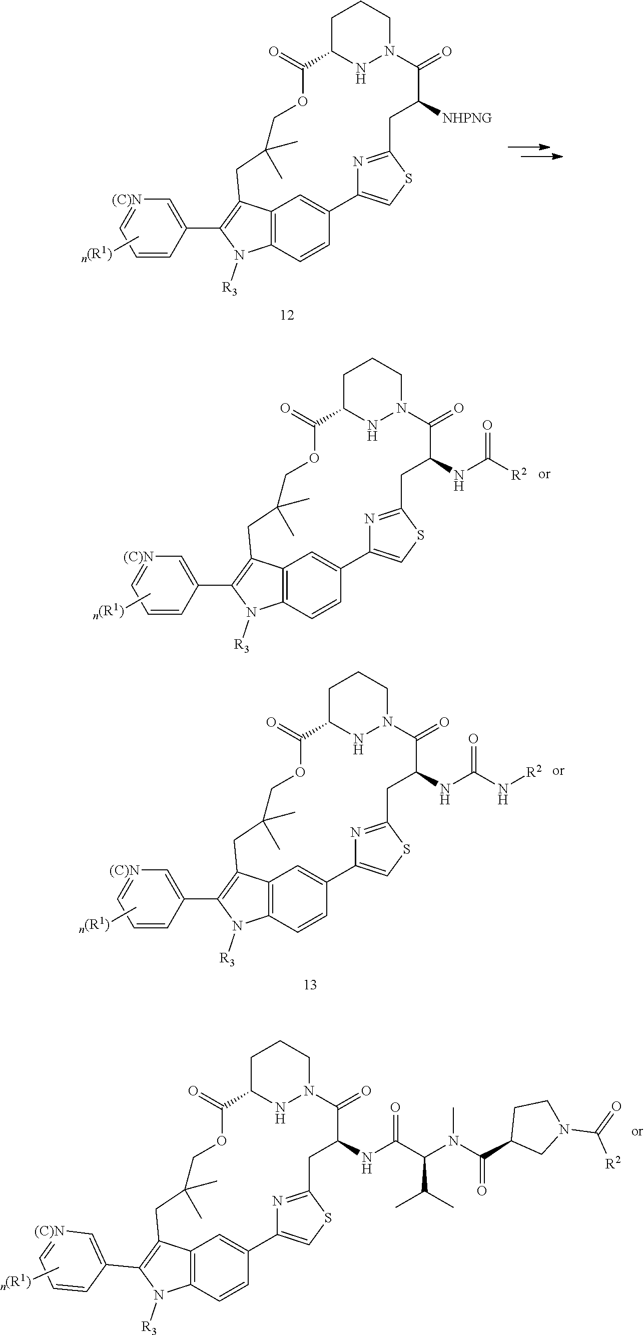

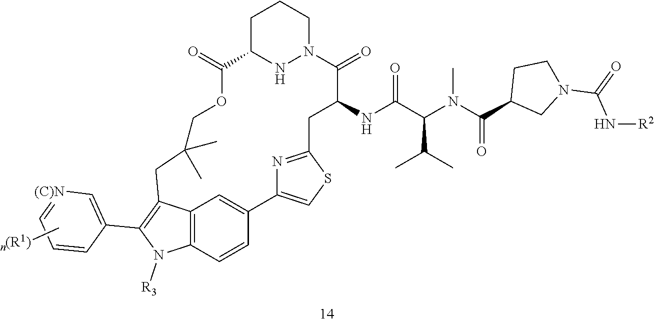

An alternative general synthesis of macrocyclic esters is outlined in Scheme A4. An appropriately substituted indolyl boronic ester (8) can be prepared in four steps starting from protected 3-(5-bromo-2-iodo-1H-indol-3-yl)-2,2-dimethylpropan-1-ol and appropriately substituted boronic acid, including Palladium mediated coupling, alkylation, de-protection, and Palladium mediated borylation reactions.

Methyl-amino-3-(4-bromothiazol-2-yl)propanoyl)hexahydropyridazine-3-carboxylate (10) can be prepared via coupling of (S)-2-amino-3-(4-bromothiazol-2-yl)propanoic acid (9) with methyl (S)-hexahydropyridazine-3-carboxylate.

The final macrocyclic esters can be made by coupling of Methyl-amino-3-(4-bromothiazol-2-yl)propanoyl)hexahydropyridazine-3-carboxylate (10) and an appropriately substituted indolyl boronic ester (8) in the presence of Pd catalyst followed by hydrolysis and macrolactonization steps to result in an appropriately protected macrocyclic intermediate (11). Deprotection and coupling with an appropriately substituted carboxylic acid (or other coupling partner) or intermediate 4 can result in a macrocyclic product. Additional deprotection or functionalization steps could be required to produce a final compound 13 or 14.

In addition, compounds of the disclosure can be synthesized using the methods described in the Examples below, together with synthetic methods known in the art of synthetic organic chemistry, or variations thereon as appreciated by those skilled in the art. These methods include but are not limited to those methods described in the WO 2021/091956. For example, a person of skill in the art would be able to install into a macrocyclic ester a desired —B-L-W group of a compound of Formula (AI), where B, L and Ware defined herein, including by using methods exemplified WO 2021/091956.

In some embodiments of the combination therapies described herein, the first RAS inhibitor is a RAS G12C inhibitor (e.g., a compound of Formula BI or a subformula thereof, or a compound of Table B1 or Table B2). RAS G12C inhibitors of Formula BI are described in WO 2021/091982, which is incorporated herein by reference in its entirety. In some embodiments, the RAS inhibitor is a compound or a pharmaceutically acceptable salt thereof, having the structure of Formula BI:

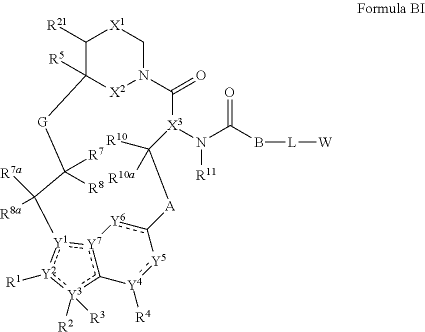

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′} C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl; or
R⁹ and R^{9′}, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R²¹ is hydrogen or C₁-C₃ alkyl (e.g., methyl).

In some embodiments of Formula BI, R⁹ is H, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula BI, R²¹ is hydrogen.

In some embodiments, the RAS inhibitor is a compound, or pharmaceutically acceptable salt thereof, having the structure of Formula BIa:

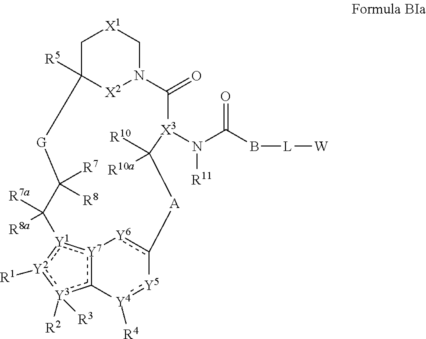

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments, the RAS inhibitor is a compound, or pharmaceutically acceptable salt thereof, of structural Formula BIb:

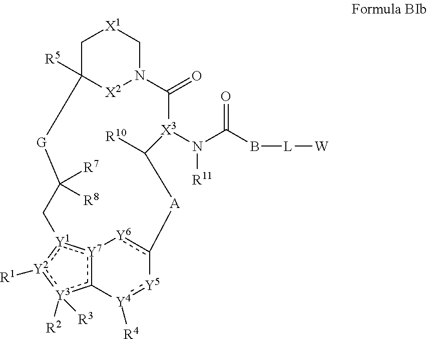

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of Formula BI and subformula thereof, G is optionally substituted C₁-C₄ heteroalkylene.

In some embodiments, the RAS inhibitor is a compound having the structure of Formula BIc, or a pharmaceutically acceptable salt thereof:

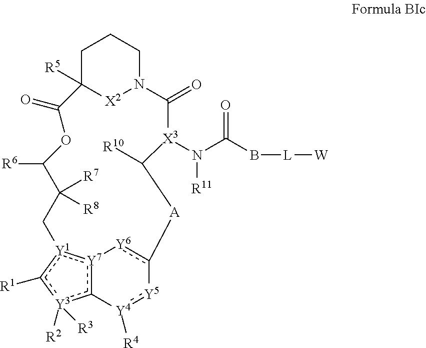

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′} C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of Formula BI and subformula thereof, X² is NH. In some embodiments of Formula BI and subformula thereof, X³ is CH. In some embodiments of Formula BI and subformula thereof, R¹¹ is hydrogen. In some embodiments of Formula BI and subformula thereof, R¹¹ is C₁-C₃ alkyl. In some embodiments of Formula BI and subformula thereof, R¹¹ is methyl.

In some embodiments, the RAS inhibitor has the structure of Formula BId, or a pharmaceutically acceptable salt thereof:

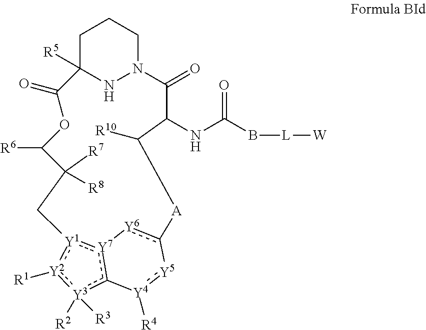

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of Formula BI and subformula thereof, X¹ is optionally substituted C₁-C₂ alkylene. In some embodiments, X¹ is methylene. In some embodiments of Formula BI and subformula thereof, X¹ is methylene substituted with a C₁-C₆ alkyl group or a halogen. In some embodiments, X¹ is —CH(Br)—. In some embodiments, X¹ is —CH(CH₃)—. In some embodiments of Formula BI and subformula thereof, R⁵ is hydrogen. In some embodiments of Formula BI and subformula thereof, R⁵ is C₁-C₄ alkyl optionally substituted with halogen. In some embodiments, R⁵ is methyl. In some embodiments of Formula BI and subformula thereof, Y⁴ is C. In some embodiments of Formula BI and subformula thereof, R⁴ is hydrogen. In some embodiments of Formula BI and subformula thereof, Y⁵ is CH.

In some embodiments of Formula BI and subformula thereof, Y⁶ is CH. In some embodiments of Formula BI and subformula thereof, Y¹ is C. In some embodiments of Formula BI and subformula thereof, Y² is C. In some embodiments of Formula BI and subformula thereof, Y³ is N. In some embodiments of Formula BI and subformula thereof, R³ is absent. In some embodiments of Formula BI and subformula thereof, Y⁷ is C.

In some embodiments, the RAS inhibitor has the structure of Formula BIe, or a pharmaceutically acceptable salt thereof:

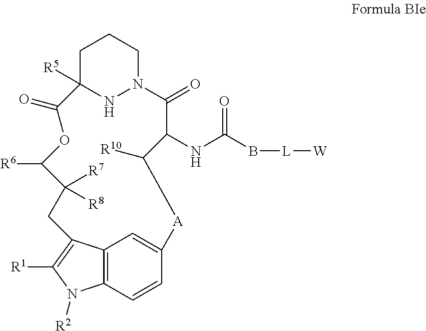

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of Formula BI and subformula thereof, R⁶ is hydrogen. In some embodiments, R² is hydrogen, cyano, optionally substituted C₁-C₆ alkyl, optionally substituted 3 to 6-membered cycloalkyl, or optionally substituted 3 to 6-membered heterocycloalkyl. In some embodiments, R² is optionally substituted C₁-C₆ alkyl. In some embodiments, R₂ is fluoroalkyl. In some embodiments, R² is ethyl. In some embodiments, R₂ is —CH₂CF₃. In some embodiments, R₂ is C₂-C₆ alkynyl. In some embodiments, R₂ is —CHC≡CH. In some embodiments, R₂ is —CH₂C≡CCH₃. In some embodiments, R⁷ is optionally substituted C₁-C₃ alkyl. In some embodiments, R⁷ is C₁-C₃ alkyl. In some embodiments, R⁸ is optionally substituted C₁-C₃ alkyl. In some embodiments, R⁸ is C₁-C₃alkyl.

In some embodiments, the RAS inhibitor has the structure of Formula BIf, or a pharmaceutically acceptable salt thereof:

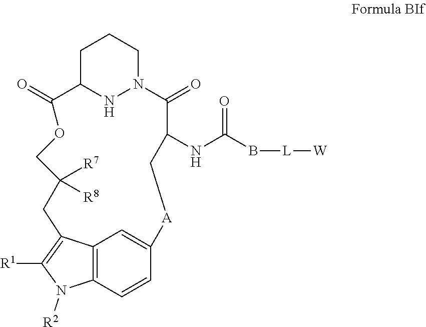

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula BI and subformula thereof, R¹ is optionally substituted 6 to 10-membered aryl, optionally substituted 3 to 6-membered cycloalkenyl, or optionally substituted 5 to 10-membered heteroaryl. In some embodiments, R¹ is optionally substituted 6-membered aryl, optionally substituted 6-membered cycloalkenyl, or optionally substituted 6-membered heteroaryl.

In some embodiments of Formula BI and subformula thereof, R₁ is

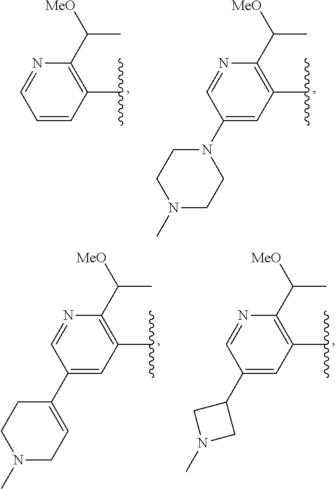 [SMILES: *c1cccnc1C(C)OC ; *c1cc(N2CCN(C)CC2)cnc1C(C)OC ; *c1cc(C2=CCN(C)CC2)cnc1C(C)OC ; *c1cc(C2CN(C)C2)cnc1C(C)OC]

In some embodiments of Formula BI and subformula thereof, R¹² is optionally substituted C₁-C₆ heteroalkyl. In some embodiments, R¹² is

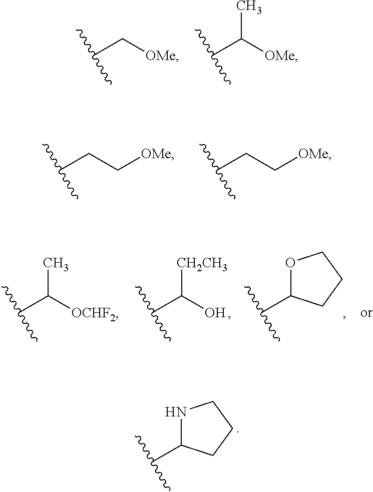

In some embodiments, R¹² is

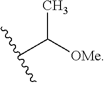 [SMILES: *C(C)OC]

In some embodiments, the RAS inhibitor has the structure of Formula BVI, or a pharmaceutically acceptable salt thereof:

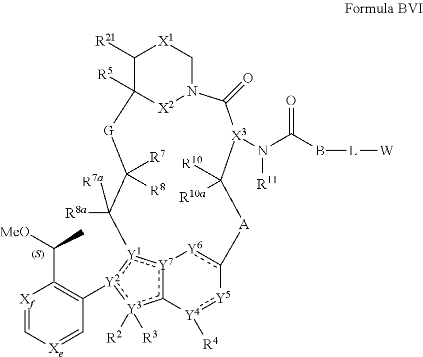

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene (e.g., phenyl or phenol), or optionally substituted 5 to 10-membered heteroarylene; B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; or
R⁹ and L combine with the atoms to which they are attached to form an optionally substituted 3 to 14-membered heterocycloalkyl;
R^{9′} is hydrogen or optionally substituted C₁-C₆ alkyl; or
R⁹ and R^{9′}, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R²¹ is hydrogen or C₁-C₃ alkyl (e.g., methyl); and
X^{e} and X^{f} are, independently, N or CH.

In some embodiments, the RAS inhibitor has the structure of Formula BVIa, or a pharmaceutically acceptable salt thereof:

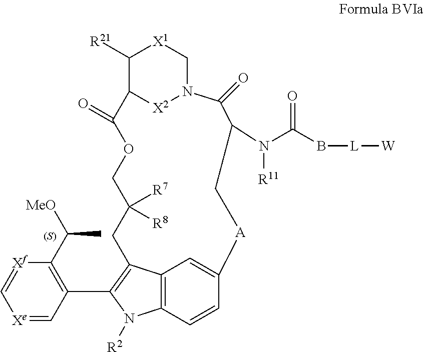

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
X¹ is optionally substituted C₁-C₂ alkylene, NR, O, or S(O)ₙ;
X² is O or NH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
R² is C₁-C₆ alkyl, C₁-C₆ fluoroalkyl, or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} and X^{f} are, independently, N or CH;
R¹¹ is hydrogen or C₁-C₃ alkyl; and
R²¹ is hydrogen or C₁-C₃ alkyl.

In some embodiments of Formula BI and subformula thereof, X^{e} is N and X^{f} is CH. In some embodiments, X^{e} is CH and X^{f} is N.

In some embodiments, the RAS inhibitor has the structure of Formula BVIb, or a pharmaceutically acceptable salt thereof:

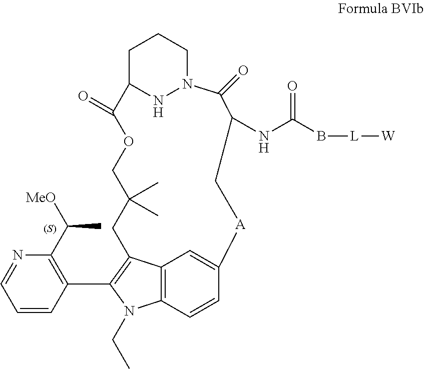

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
L is absent or a linker; and
W is a cross-linking group comprising a vinyl ketone, a vinyl sulfone, an ynone, or an alkynyl sulfone.

In some embodiments of formula BI or subformula thereof, A is optionally substituted 6-membered arylene.

In some embodiments, the RAS inhibitor has the structure of Formula BVIc, or a pharmaceutically acceptable salt thereof:

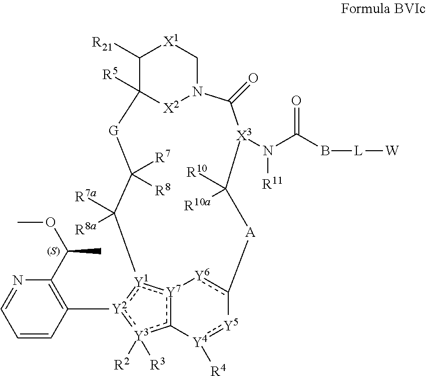

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene (e.g., phenyl or phenol), or optionally substituted 5 to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂; each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′} C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; or
R⁹ and L combine with the atoms to which they are attached to form an optionally substituted 3 to 14-membered heterocycloalkyl;
R^{9′} is hydrogen or optionally substituted C₁-C₆ alkyl; or
R⁹ and R^{9′}, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R²¹ is hydrogen or C₁-C₃ alkyl (e.g., methyl).

In some embodiments of Formula BI and subformula thereof, A has the structure:

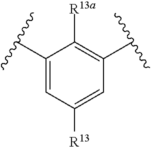

wherein R¹³ is hydrogen, halo, hydroxy, amino, optionally substituted C₁-C₆ alkyl, or optionally substituted C₁-C₆ heteroalkyl; and R^{13a} is hydrogen or halo. In some embodiments, R¹³ is hydrogen. In some embodiments, R¹³ and R^{13a} are each hydrogen. In some embodiments, R¹³ is hydroxy, methyl, fluoro, or difluoromethyl.

In some embodiments of Formula BI and subformula thereof, A is optionally substituted 5 to 6-membered heteroarylene. In some embodiments, A is:

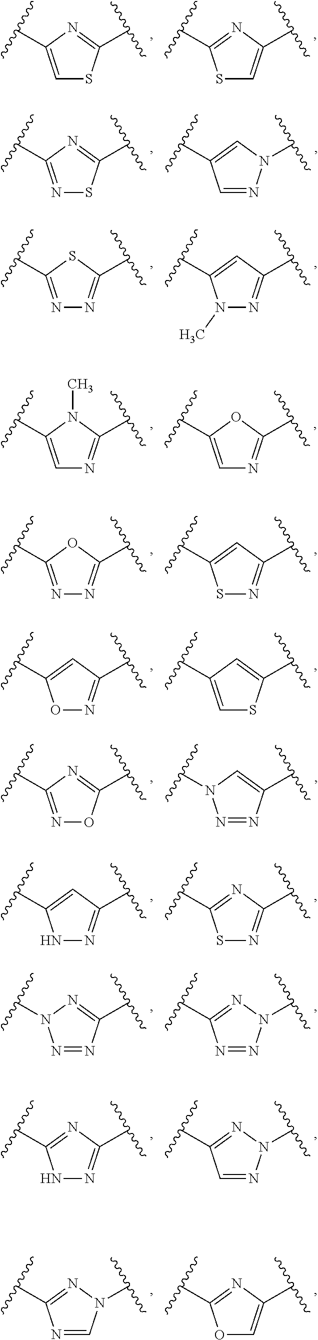

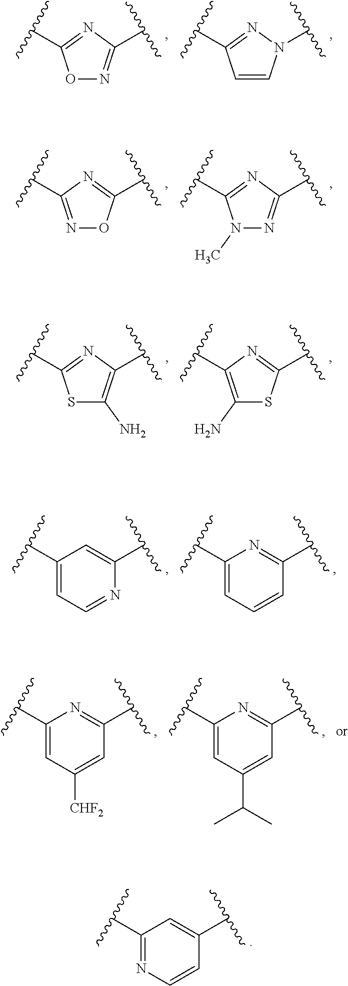

In some embodiments of Formula BI and subformula thereof, A is optionally substituted C₁-C₄ heteroalkylene. In some embodiments, A is:

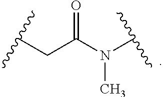 [SMILES: *CC(=O)N(*)C]

In some embodiments of Formula BI and subformula thereof, A is optionally substituted 3 to 6-membered heterocycloalkylene. In some embodiments, A is:

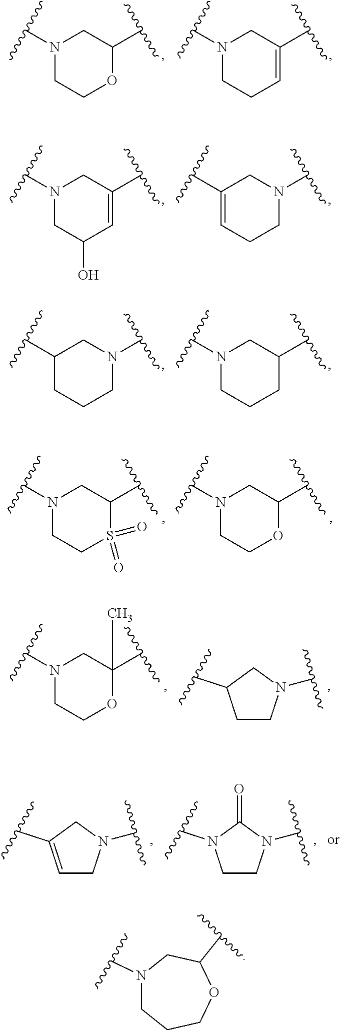

In some embodiments, A is

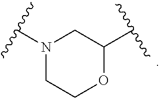 [SMILES: *C1CN(*)CCO1]

In some embodiments of Formula BI and subformula thereof, B is —CHR⁹—. In some embodiments of Formula BI and subformula thereof, R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl. In some embodiments, R⁹ is:

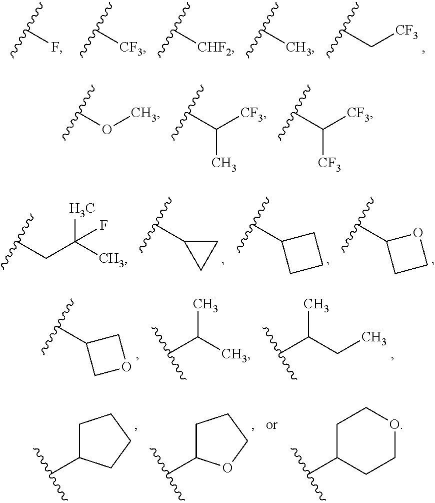

In some embodiments, R⁹ is:

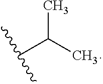 [SMILES: *C(C)C]

In some embodiments, R⁹ is H, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula BI and subformula thereof, B is optionally substituted 6-membered arylene. In some embodiments, B is 6-membered arylene. In some embodiments, B is:

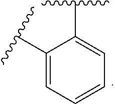 [SMILES: *c1ccccc1*]

In some embodiments of Formula BI and subformula thereof, R⁷ is methyl.

In some embodiments of Formula BI and subformula thereof, R⁸ is methyl.

In some embodiments of Formula BI and subformula thereof, R²¹ is hydrogen.

In some embodiments of Formula BI and subformula thereof, the linker is the structure of Formula BII:

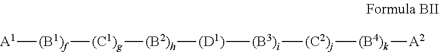

where A¹ is a bond between the linker and B; A² is a bond between Wand the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁₋₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to —(B³)ᵢ—(C²)ⱼ—(B⁴)ₖ-A². In some embodiments, the linker is acyclic. In some embodiments, linker has the structure of Formula BIIa:

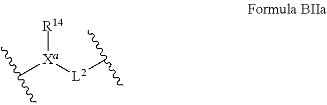

wherein X^{a} is absent or N;
R¹⁴ is absent, hydrogen or optionally substituted C₁-C₆ alkyl; and
L² is absent, —SO₂—, optionally substituted C₁-C₄ alkylene or optionally substituted C₁-C₄ heteroalkylene, wherein at least one of X^{a}, R¹⁴, or L² is present. In some embodiments, the linker has the structure:

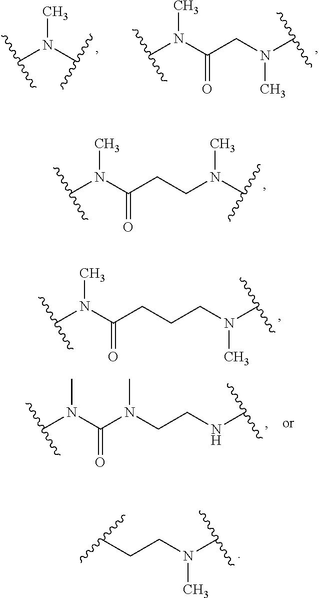

In some embodiments of Formula BI and subformula thereof, the linker is or comprises a cyclic moiety. In some embodiments, the linker has the structure of Formula BIIb:

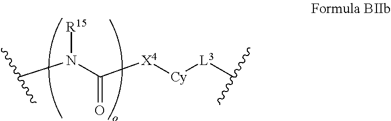

wherein o is 0 or 1;
R¹⁵ is hydrogen or optionally substituted C₁-C₆ alkyl, optionally substituted 3 to 8-membered cycloalkylene, or optionally substituted 3 to 8-membered heterocycloalkylene;
X⁴ is absent, optionally substituted C₁-C₄ alkylene, O, NCH₃, or optionally substituted C₁-C₄ heteroalkylene;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —SO₂—, optionally substituted C₁-C₄ alkylene or optionally substituted C₁-C₄ heteroalkylene.

In some embodiments of Formula BI and subformula thereof, the linker has the structure of Formula BIIb-1:

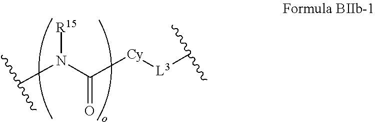

wherein o is 0 or 1;
R¹⁵ is hydrogen or optionally substituted C₁-C₆ alkyl, optionally substituted 3 to 8-membered cycloalkylene, or optionally substituted 3 to 8-membered heterocycloalkylene;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —SO₂—, optionally substituted C₁-C₄ alkylene or optionally substituted C₁-C₄ heteroalkylene.

In some embodiments of Formula BI and subformula thereof, the linker has the structure of Formula BIIc:

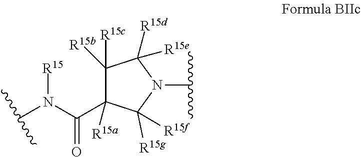

wherein R¹⁵ is hydrogen, optionally substituted C₁-C₆ alkyl, optionally substituted 3 to 8-membered cycloalkylene, or optionally substituted 3 to 8-membered heterocycloalkylene; and
R^{15a}, R^{15b}, R^{15c}, R^{15d}, R^{15e}, R^{15f}, and R^{15g} are, independently, hydrogen, halo, hydroxy, cyano, amino, optionally substituted C₁-C₆ alkyl, optionally substituted C₁-C₆ alkoxy, or, or R^{15b} and R^{15d} combine with the carbons to which they are attached to form an optionally substituted 3 to 8-membered cycloalkylene, or optionally substituted 3 to 8-membered heterocycloalkylene.

In some embodiments of Formula BI and subformula thereof, the linker has the structure:

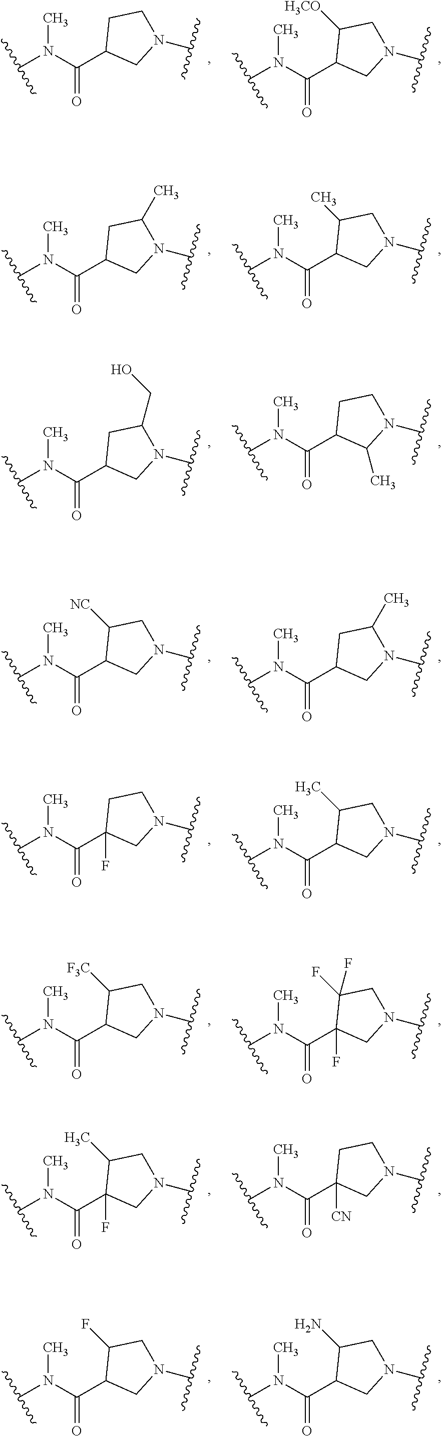

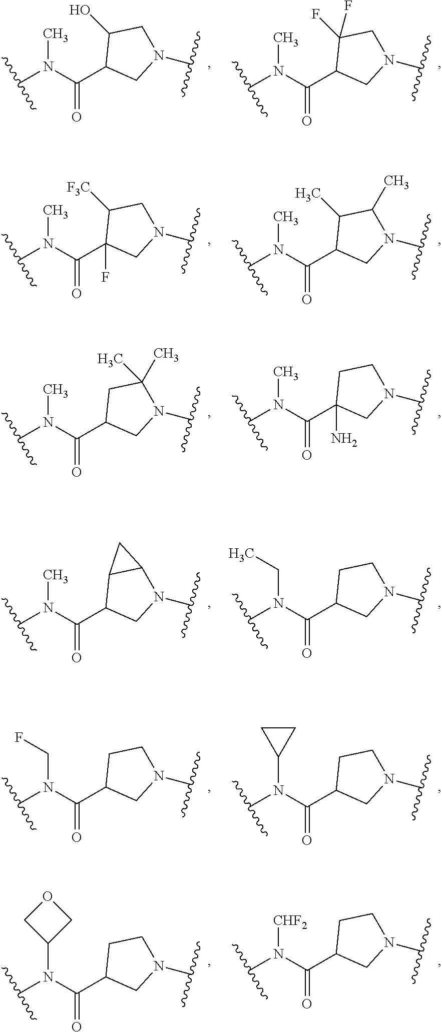

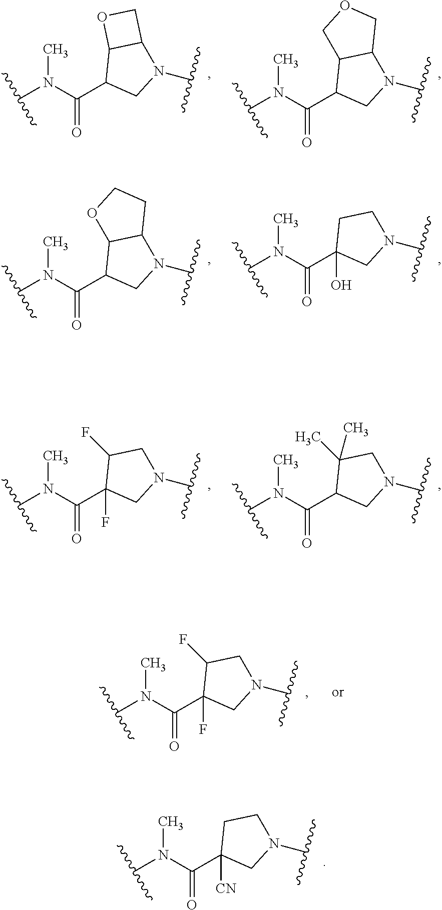

In some embodiments of Formula BI and subformula thereof, the linker has the structure:

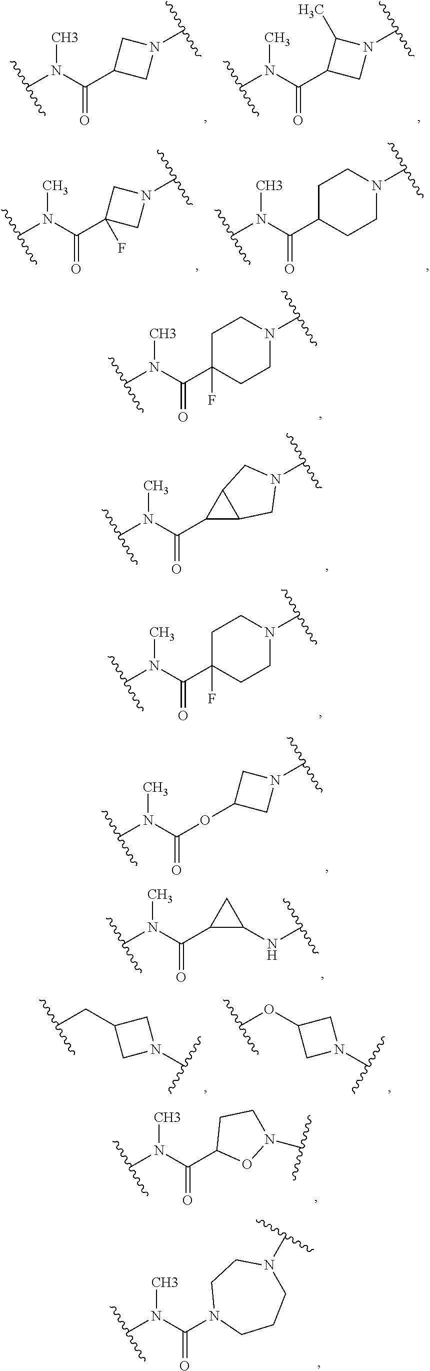

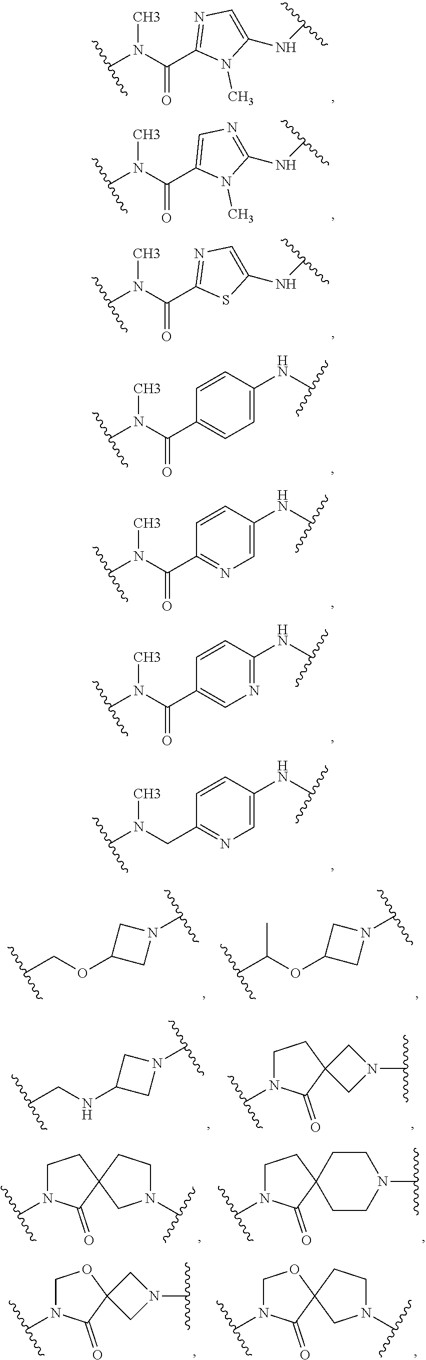

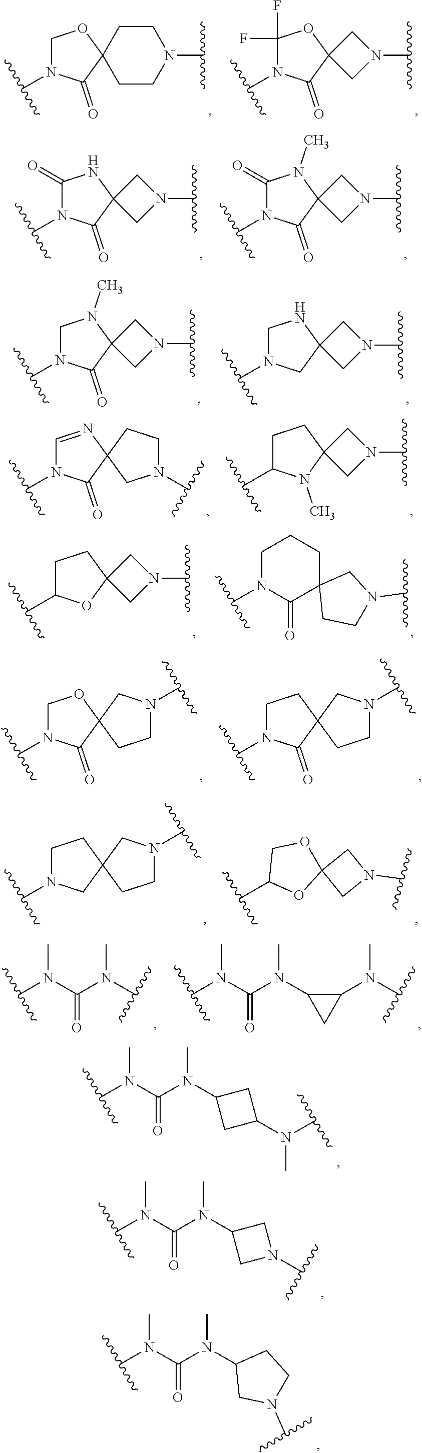

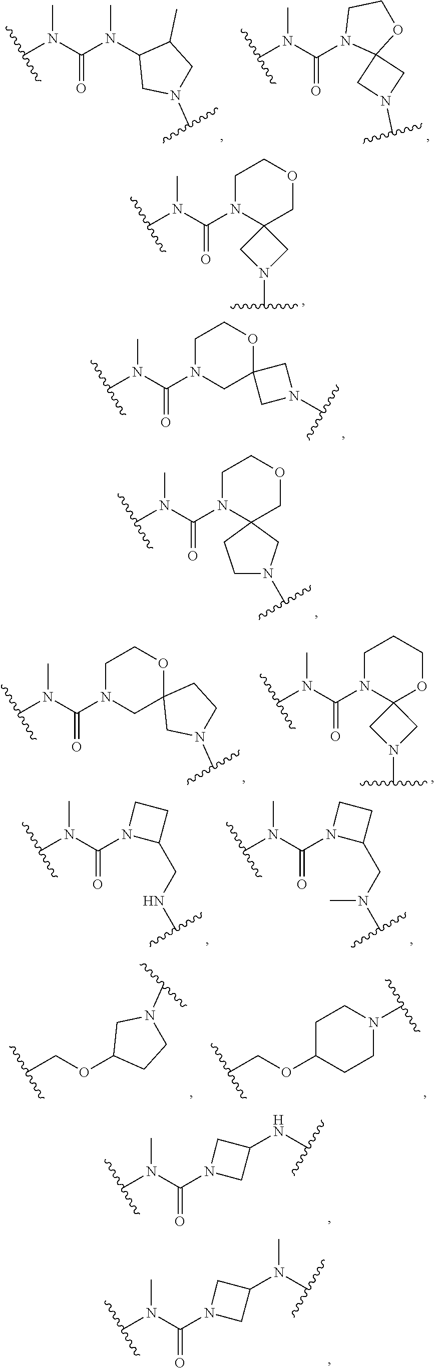

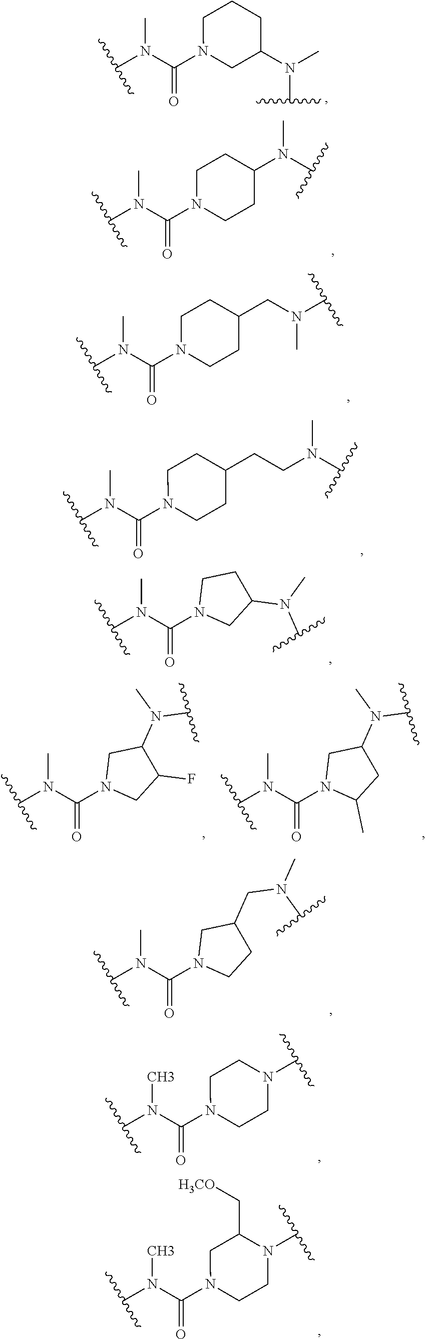

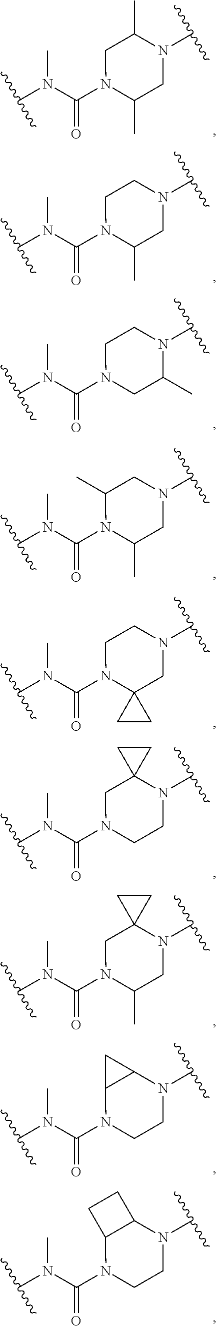

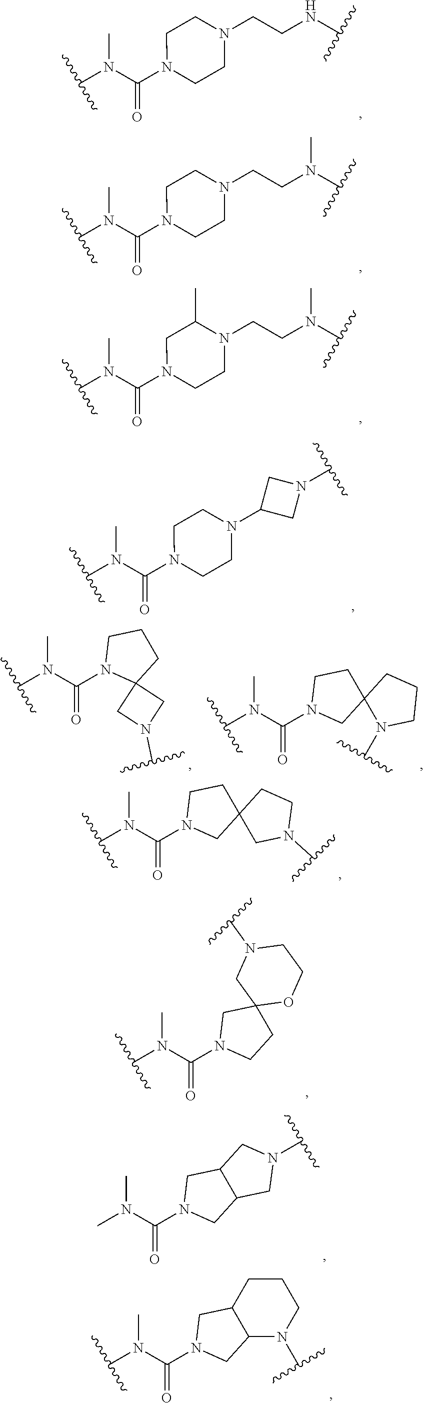

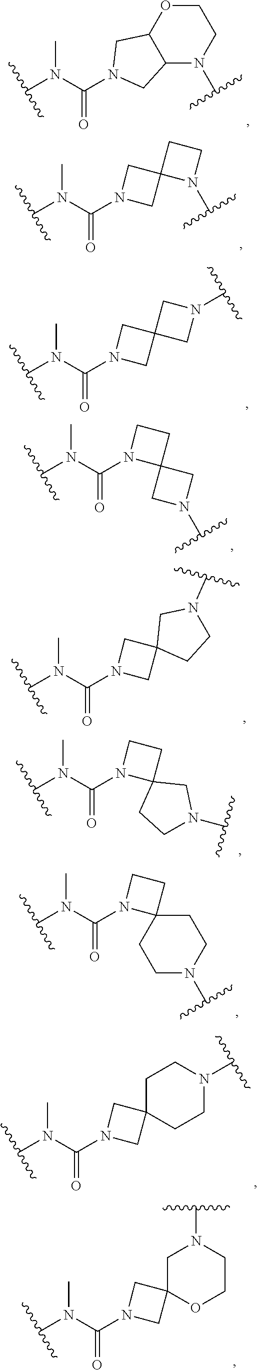

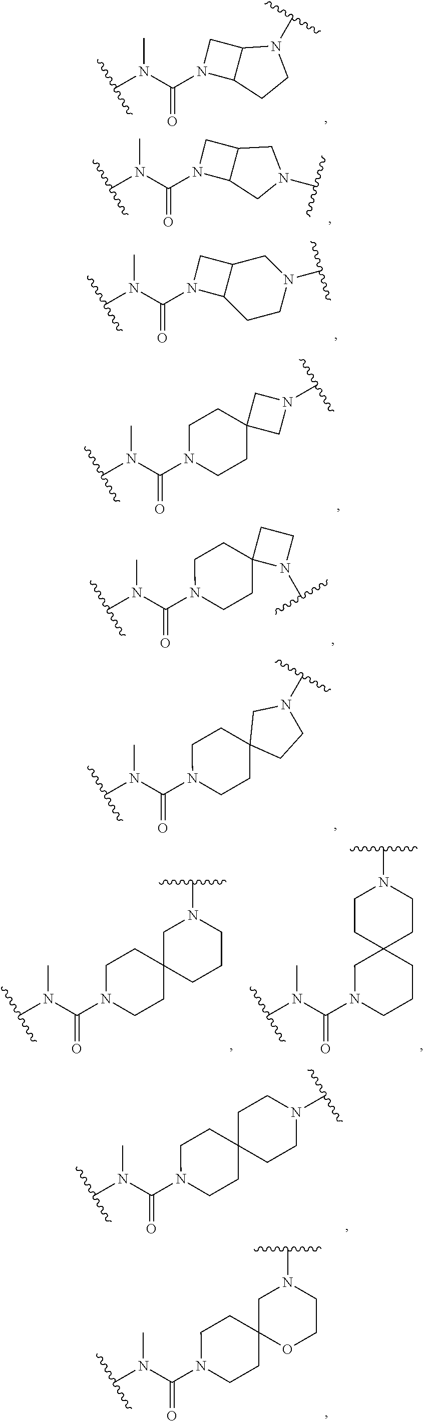

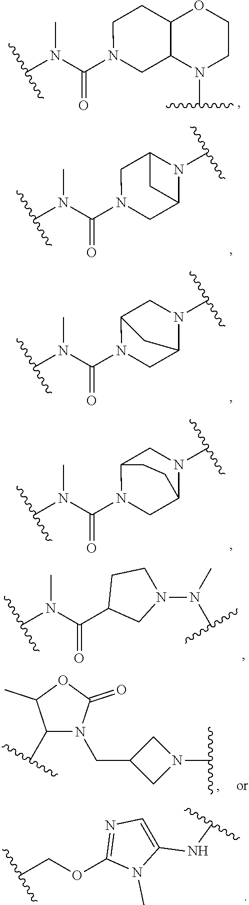

In some embodiments of Formula BI and subformula thereof, the linker has the structure

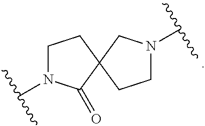 [SMILES: *N1CCC2(CCN(*)C2=O)C1]

In some embodiments of Formula BI and subformula thereof, the linker has the structure

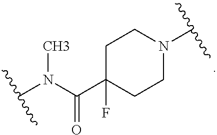 [SMILES: *N1CCC(F)(C(=O)N(*)C)CC1]

In some embodiments of Formula BI and subformula thereof, W is a cross-linking group comprising a vinyl ketone. In some embodiments, W has the structure of Formula BIIIa:

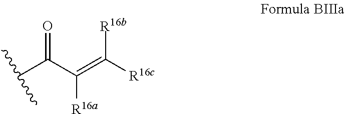

wherein R^{16a}, R^{16b}, and R^{16c} are, independently, hydrogen, —CN, halogen, or —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl. In some embodiments, W is:

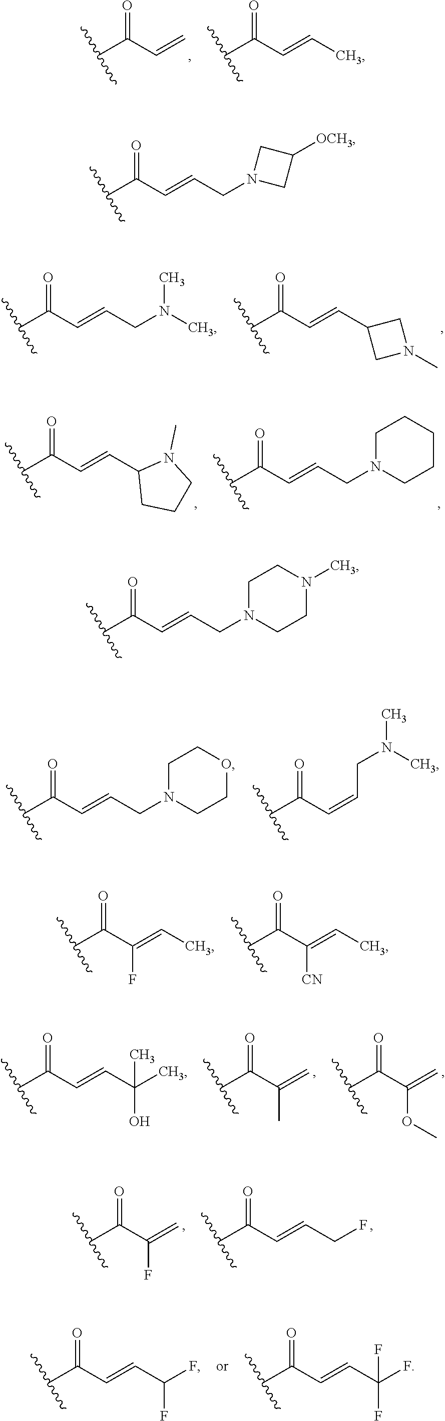

In some embodiments of Formula BI and subformula thereof, W is a cross-linking group comprising an ynone. In some embodiments, W has the structure of Formula BIIIb:

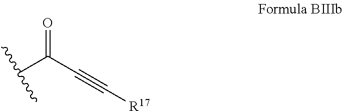

wherein R¹⁷ is hydrogen, —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl, or a 4 to 7-membered saturated heterocycloalkyl. In some embodiments, W is:

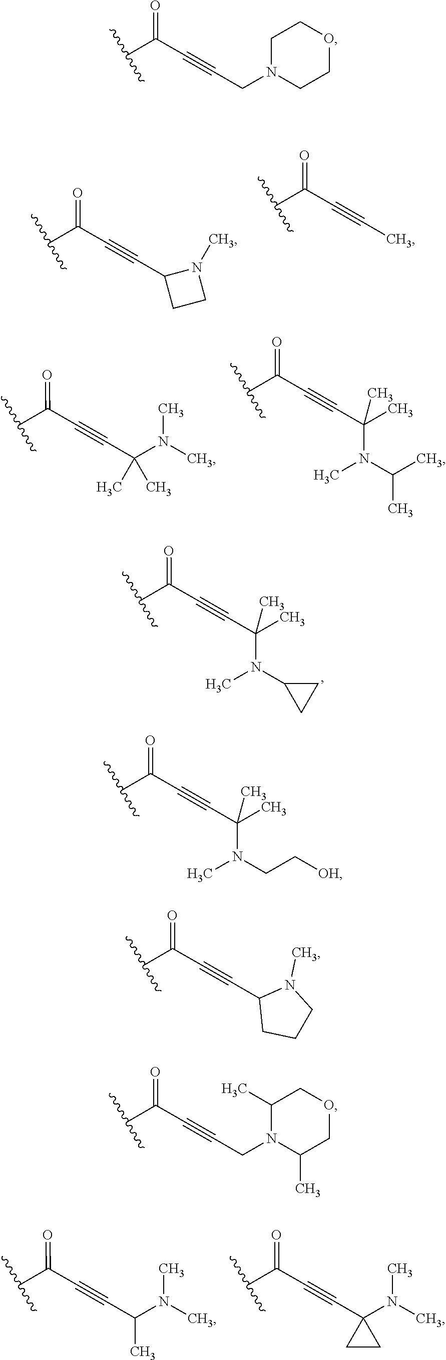

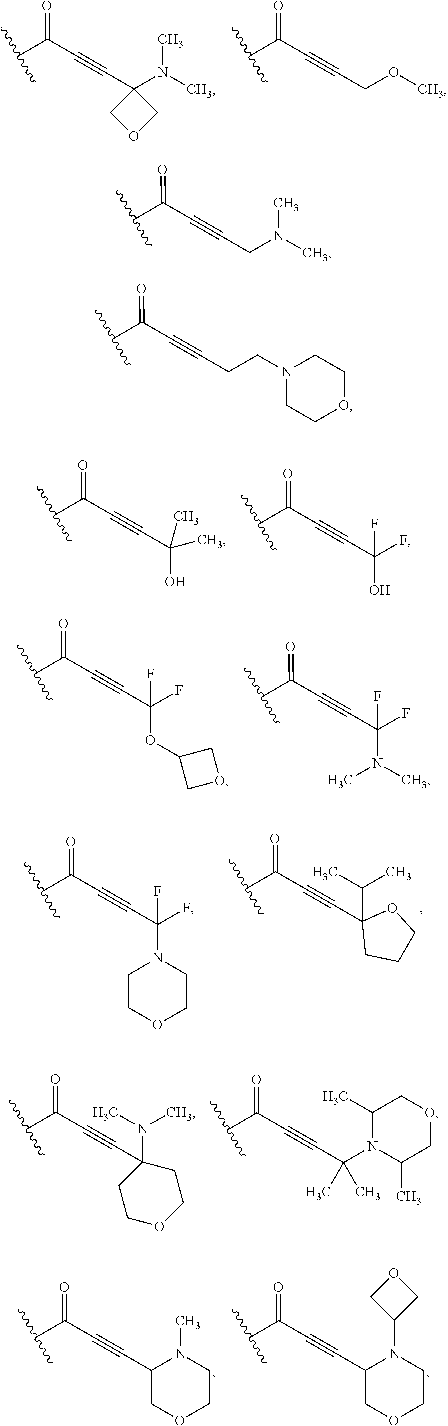

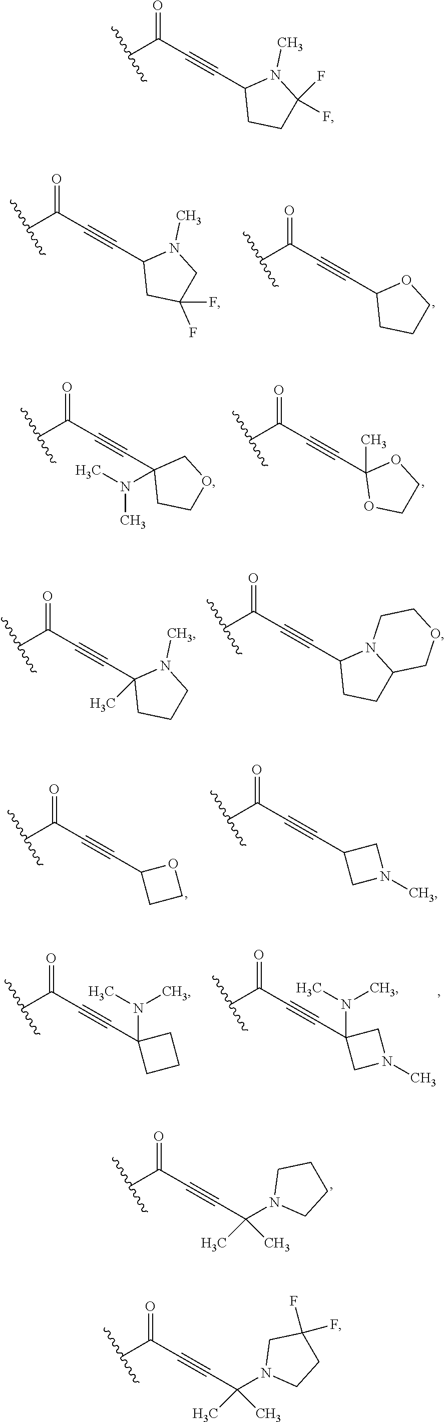

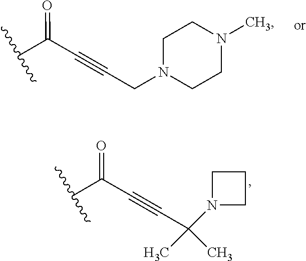 [SMILES: *C(=O)C#CCN1CCN(C)CC1 ; *C(=O)C#CC(C)(C)N1CCC1]

In some embodiments, W is

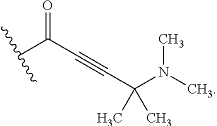 [SMILES: *C(=O)C#CC(C)(C)N(C)C]

In some embodiments of Formula BI and subformula thereof, W is a cross-linking group comprising a vinyl sulfone. In some embodiments, W has the structure of Formula BIIIc:

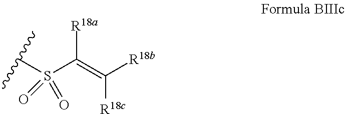

wherein R^{18a}, R^{18b}, and R^{18c} are, independently, hydrogen, —CN, or —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl. In some embodiments, W is:

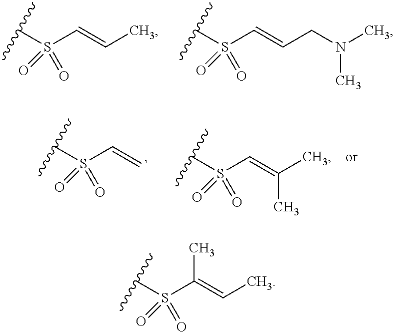

In some embodiments of Formula BI and subformula thereof, W is a cross-linking group comprising an alkynyl sulfone. In some embodiments, W has the structure of Formula BIIId:

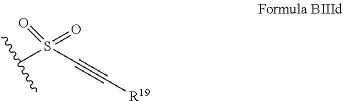

wherein R¹⁹ is hydrogen, —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl, or a 4 to 7-membered saturated heterocycloalkyl. In some embodiments, W is:

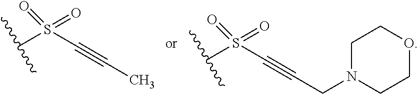

In some embodiments of Formula BI and subformula thereof, W has the structure of Formula BIIIe:

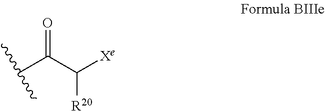

wherein X^{e} is a halogen; and
R²⁰ is hydrogen, —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl. In some embodiments of Formula BI and subformula thereof, W is haloacetyl. In some embodiments of Formula BI and subformula thereof, W is not haloacetyl.

In some embodiments, the RAS inhibitor is a RAS G12C inhibitor. In some embodiments, the RAS G12C inhibitor is selected from Table B1, or a pharmaceutically acceptable salt or stereoisomer thereof. In some embodiments, the RAS G12C inhibitor is selected from Table B1, or a pharmaceutically acceptable salt or atropisomer thereof.

TABLE B1
Certain Compounds of the Present Invention

| Ex# | Structure |
|---|---|
| BA1 | 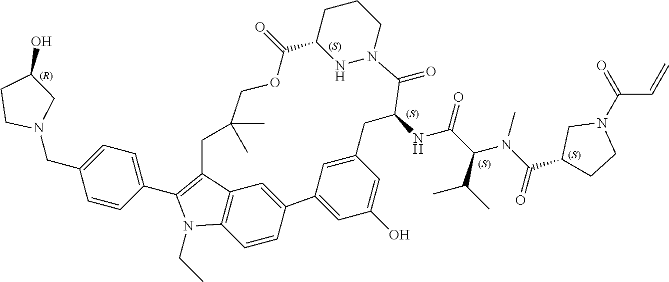 |
| BA2 | 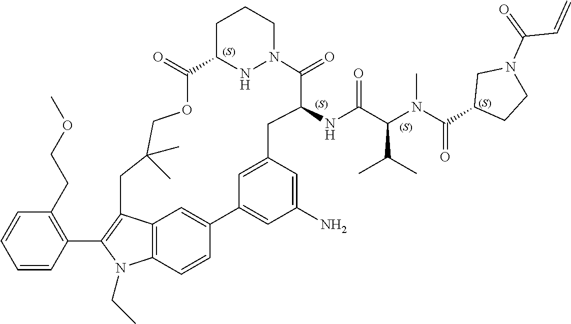 |
| BA3 | 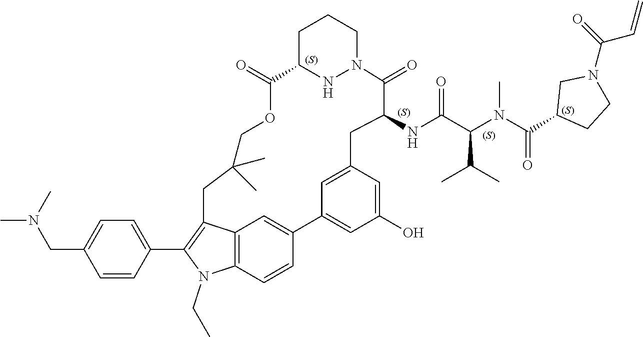 |
| BA4 | 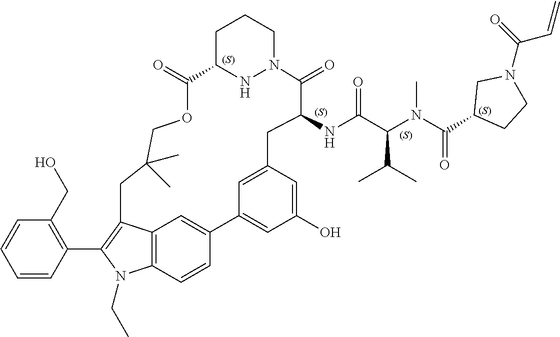 |
| BA5 | 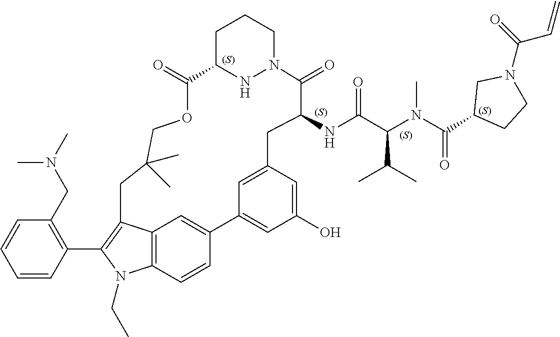 |
| BA6 | 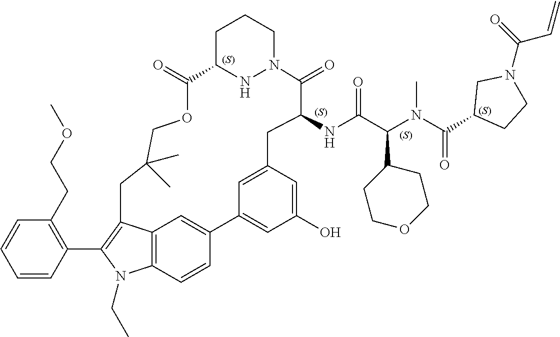 |
| BA7 | 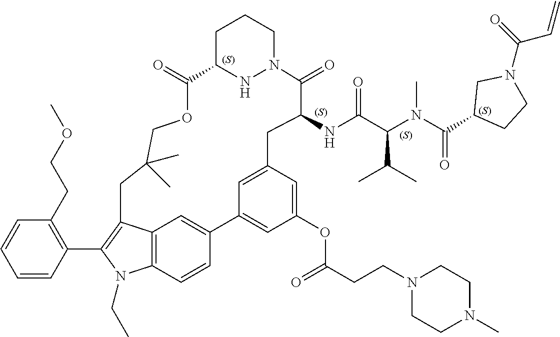 |
| BA8 | 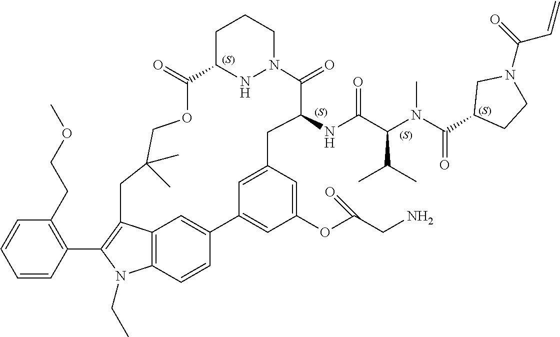 |
| BA9 | 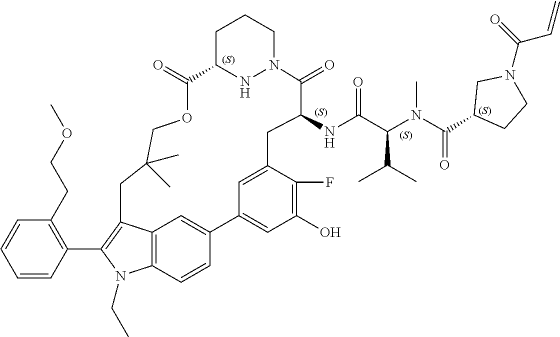 |
| BA10 | 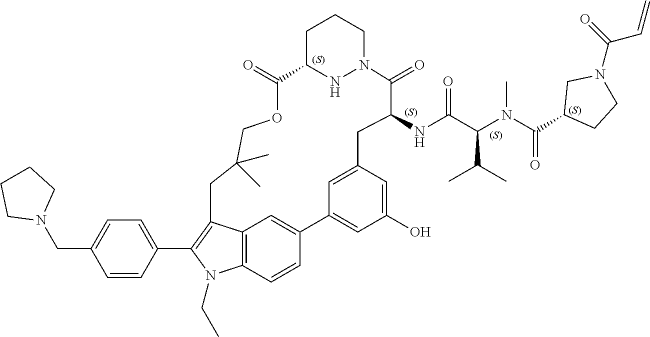 |
| BA11 | 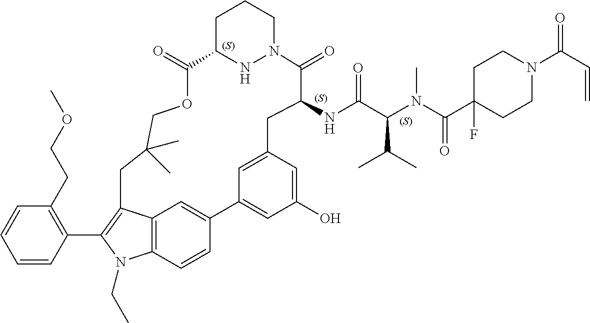 |
| BA12 | 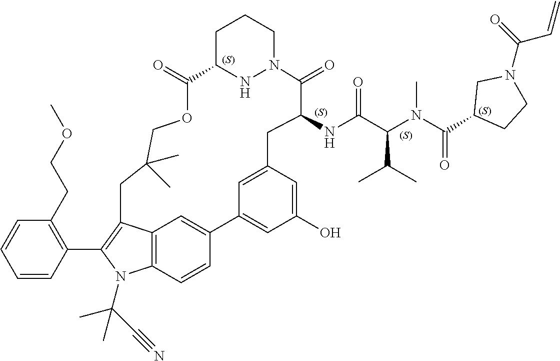 |
| BA13 | 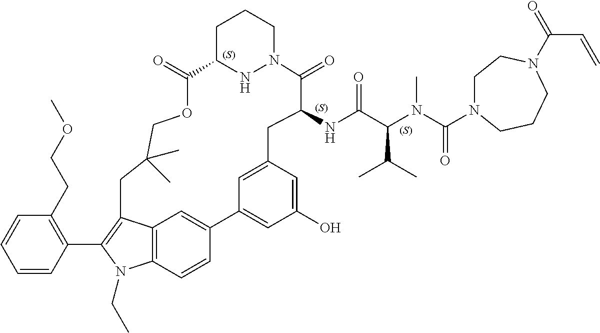 |
| BA14 | 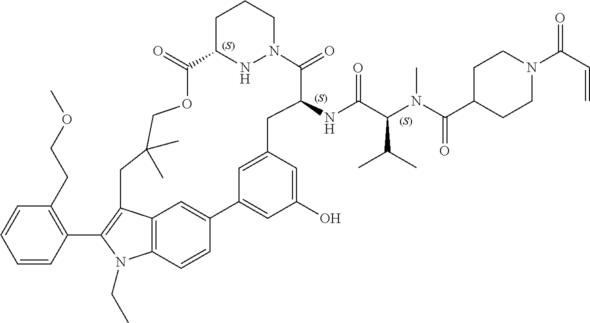 |
| BA15 | 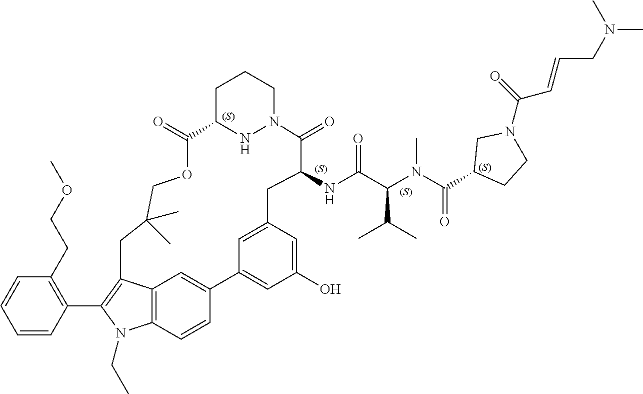 |
| BA16 | 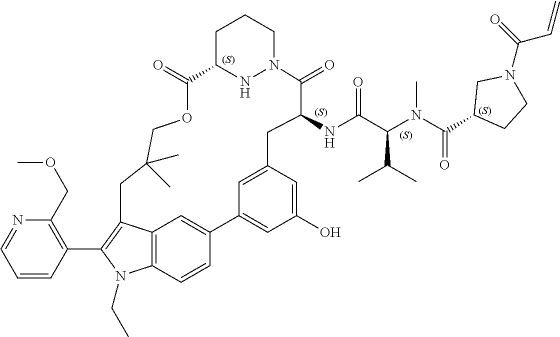 |
| BA17 | 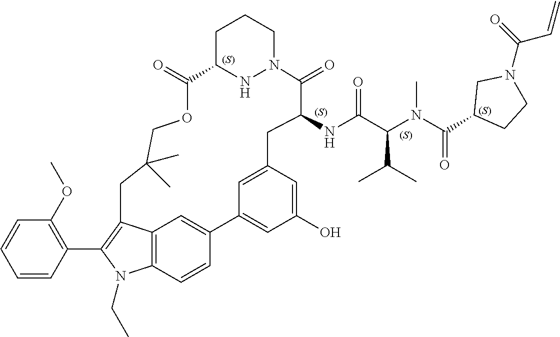 |
| BA18 | 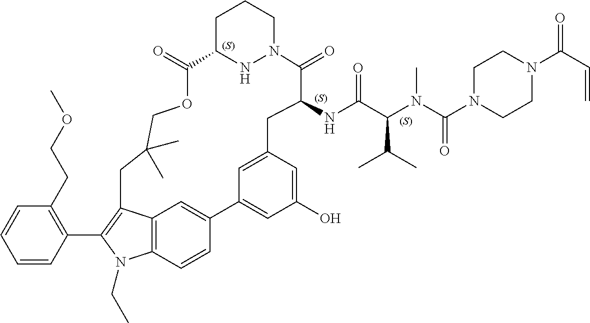 |
| BA19 | 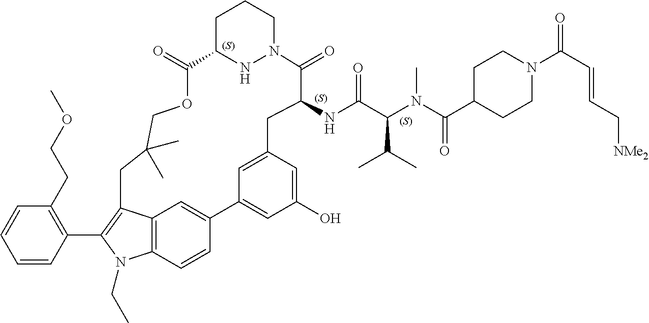 |
| BA20 | 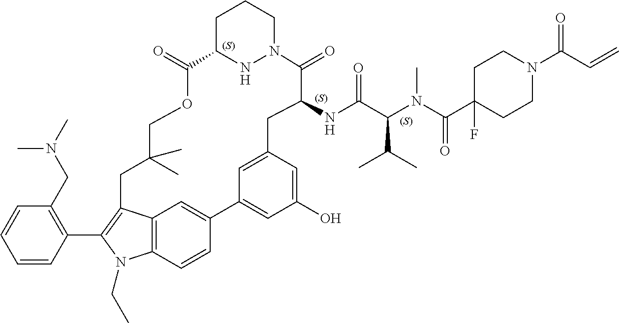 |
| BA21 | 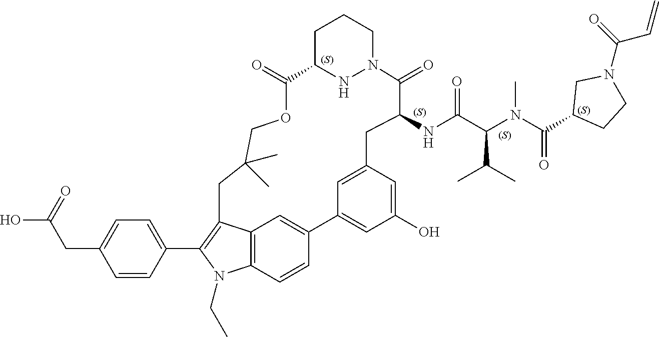 |
| BA22 | 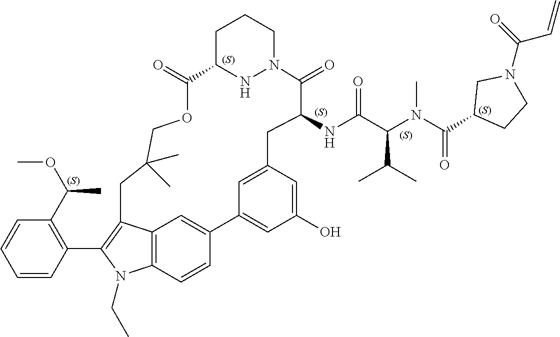 |
| BA23 | 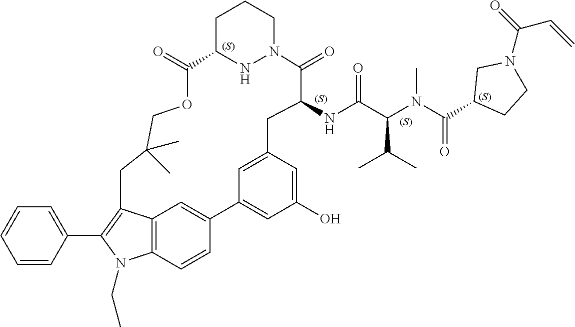 |
| BA24 | 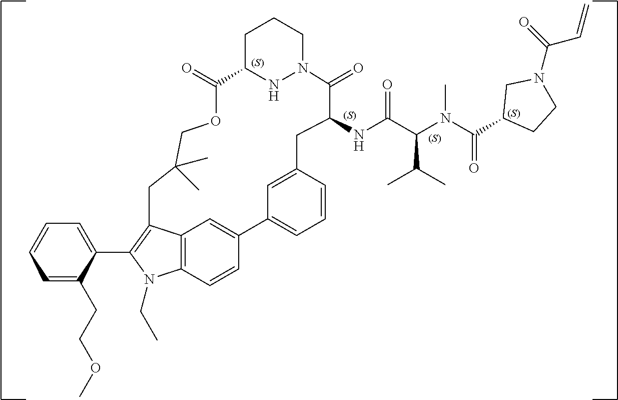 |
| BA25 | 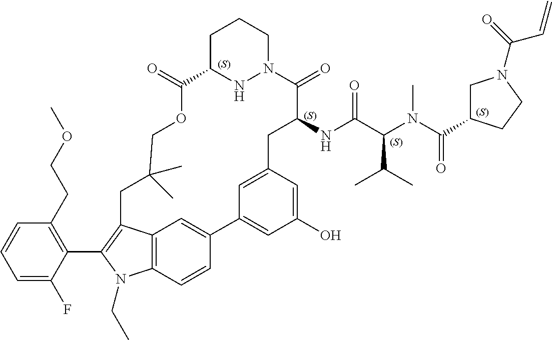 |
| BA26 | 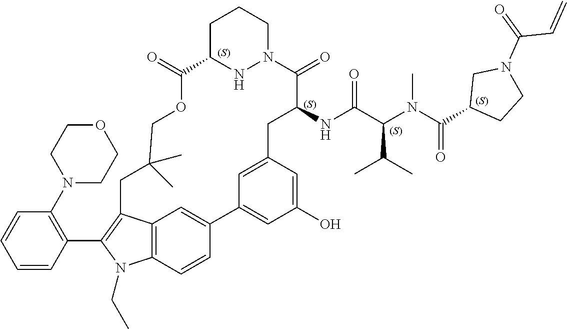 |
| BA27 | 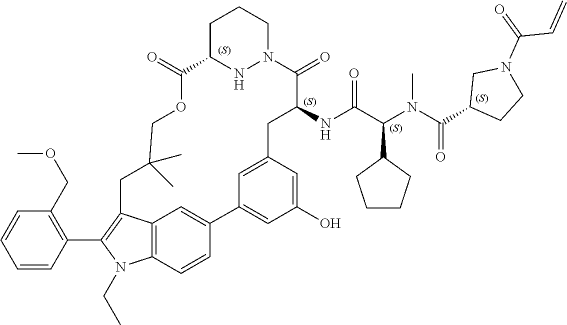 |
| BA28 | 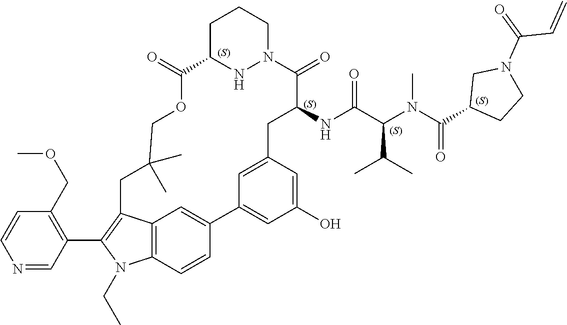 |
| BA29 | 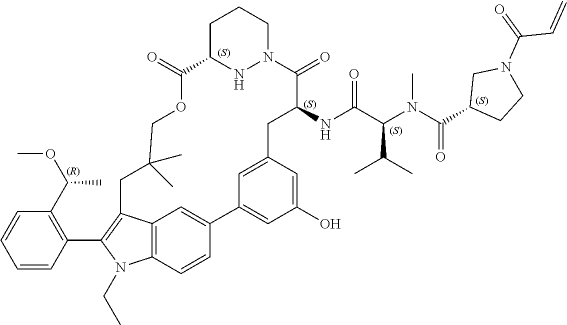 |
| BA30 | 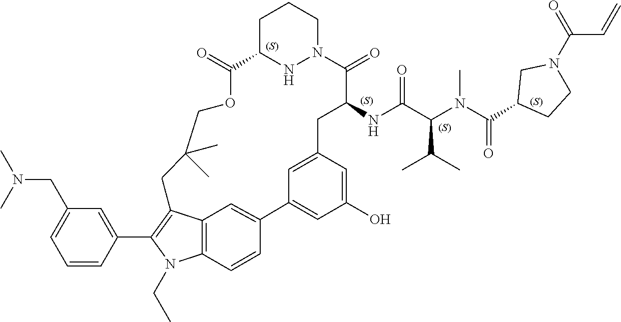 |
| BA31 | 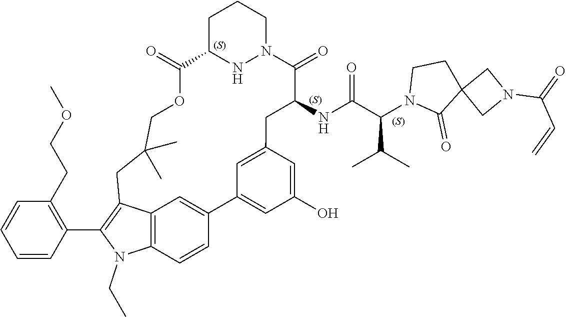 |
| BA32 | 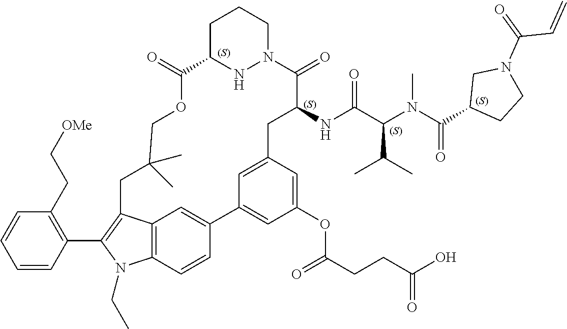 |
| BA33 | 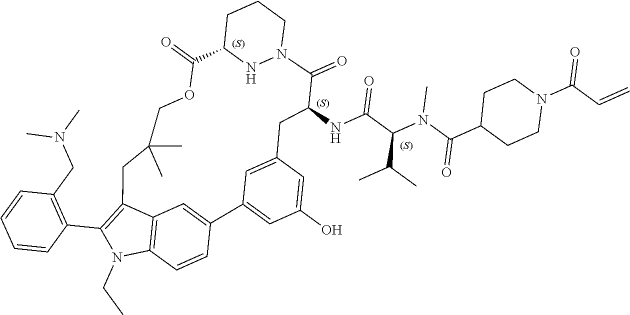 |
| BA34 | 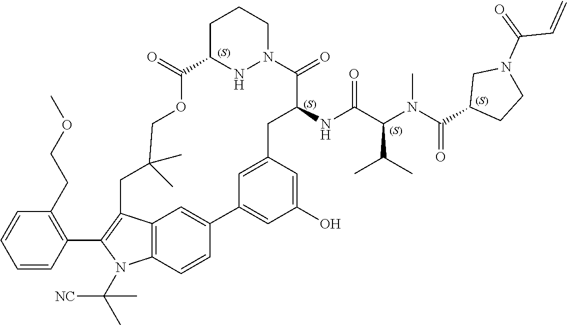 |
| BA35 | 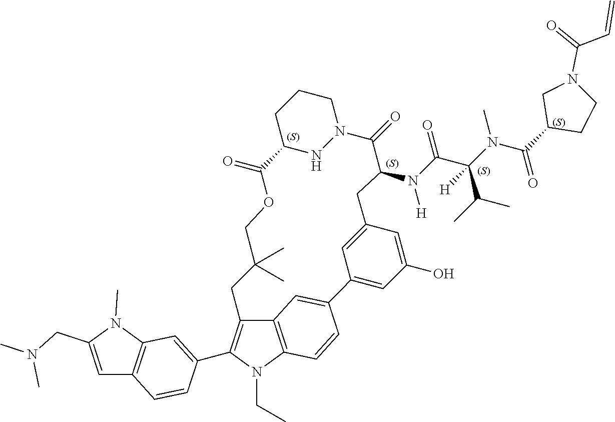 |
| BA36 | 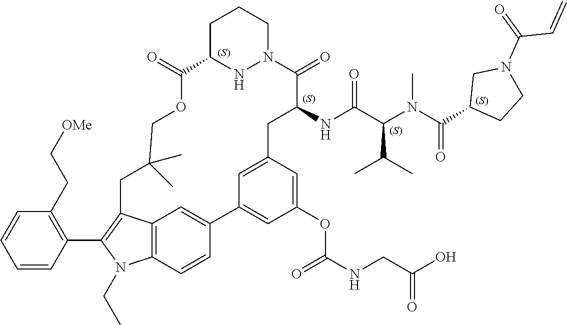 |
| BA37 | 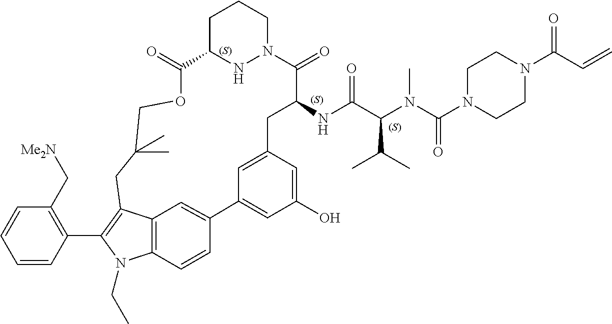 |
| BA38 | 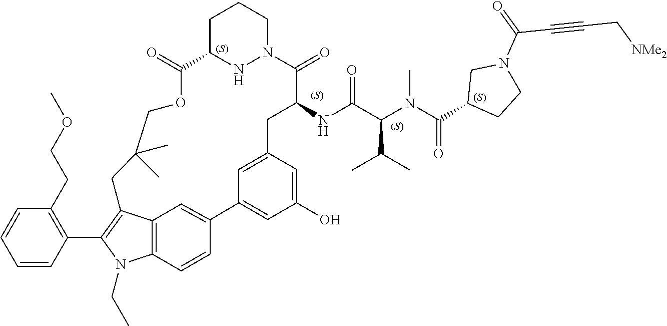 |
| BA39 | 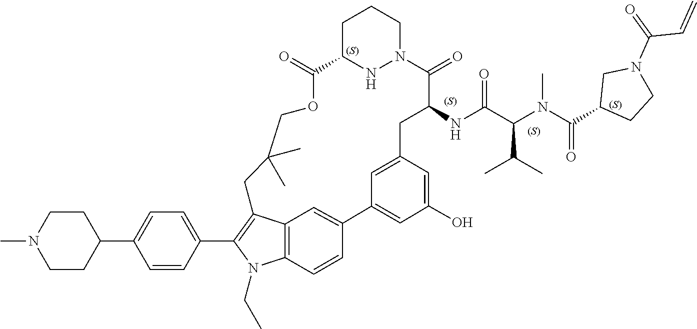 |
| BA40 | 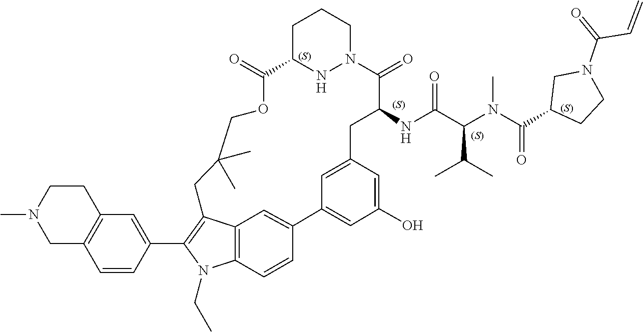 |
| BA41 | 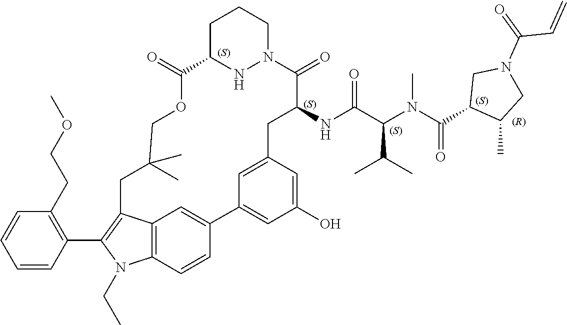 |
| BA42 | 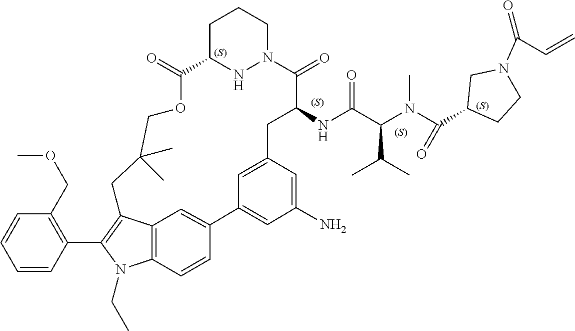 |
| BA43 | 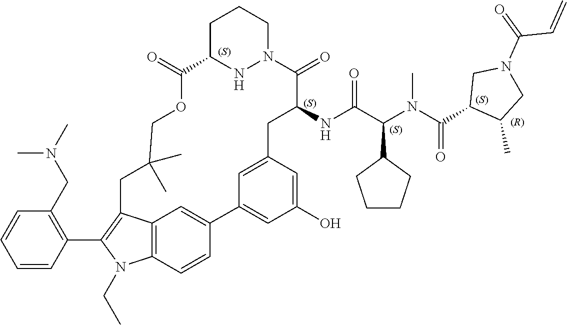 |
| BA44 | 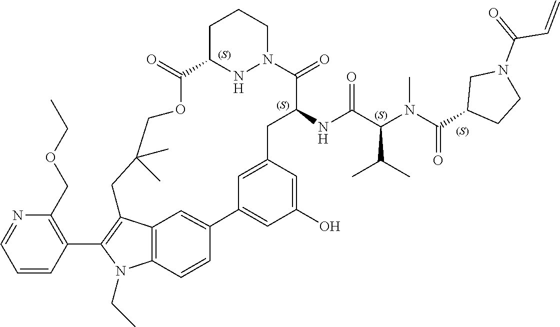 |
| BA45 | 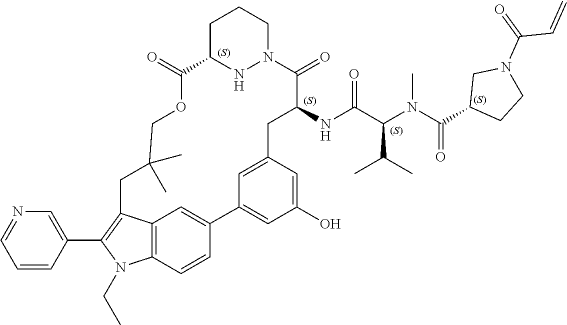 |
| BA46 | 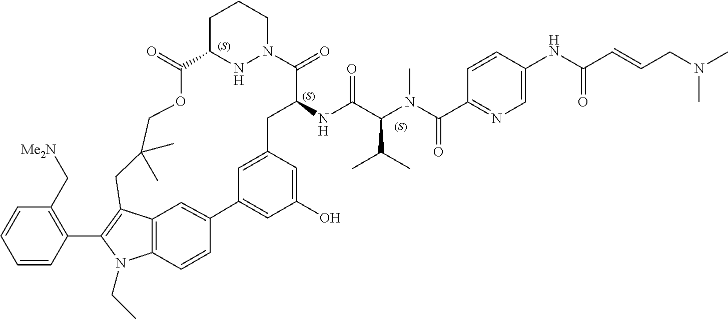 |
| BA47 | 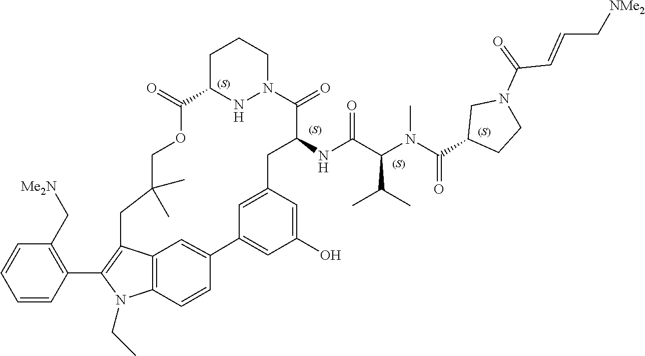 |
| BA48 | 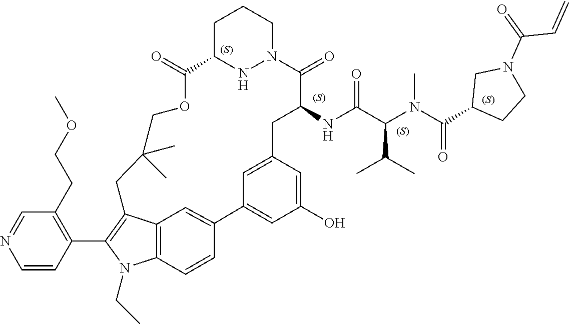 |
| BA49 | 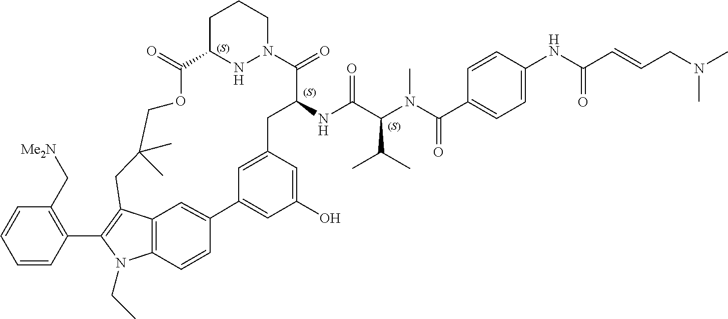 |
| BA50 | 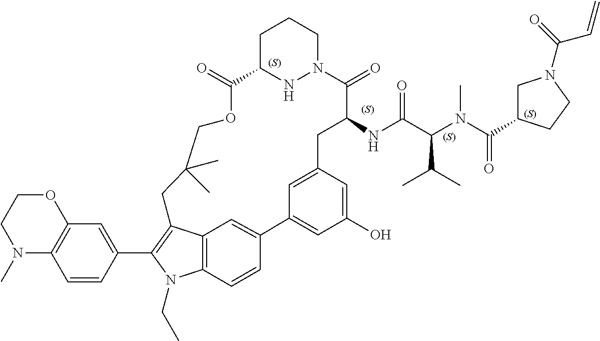 |
| BA51 | 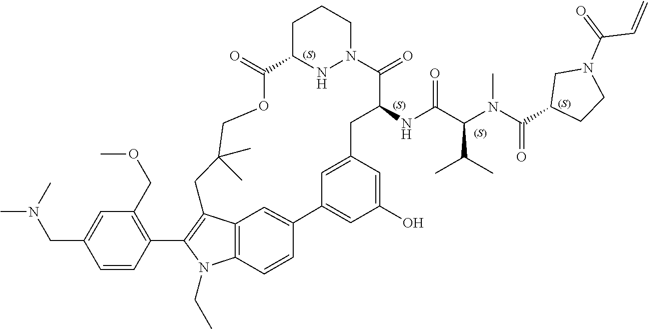 |
| BA52 | 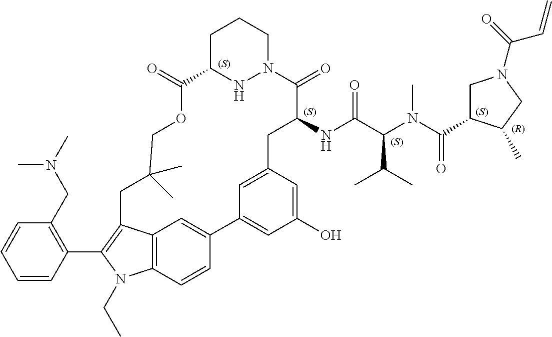 |
| BA53 | 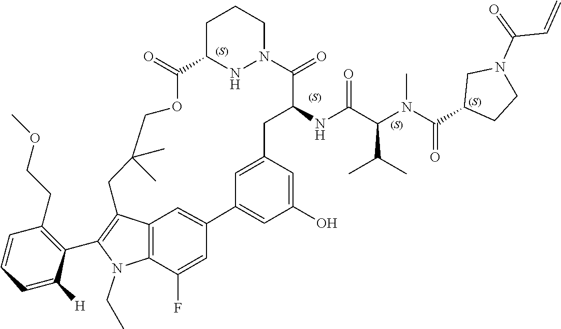 |
| BA54 | 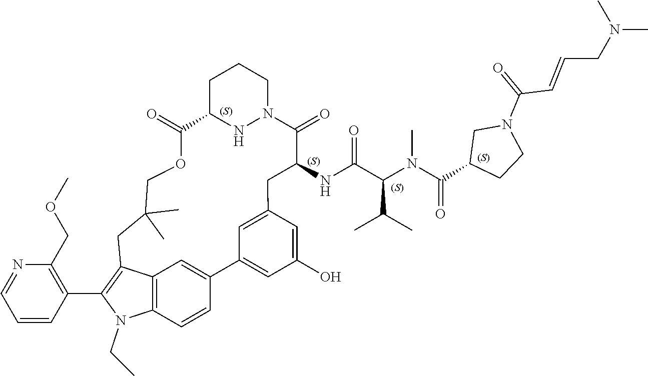 |
| BA55 | 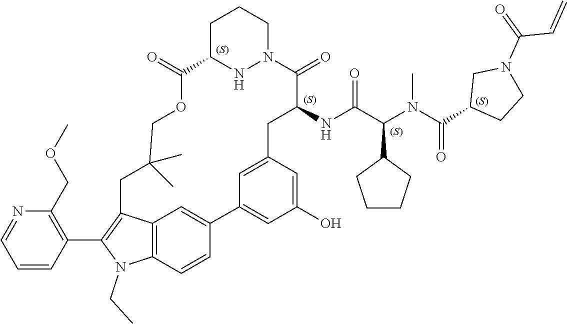 |
| BA56 | 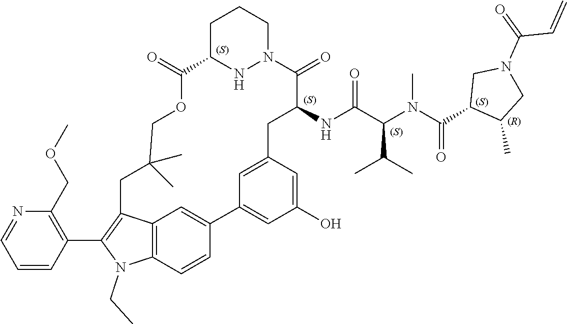 |
| BA57 | 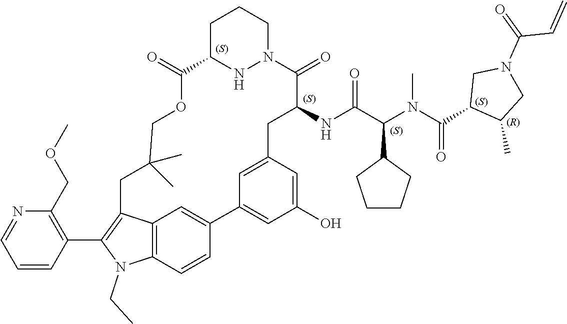 |
| BA58 | 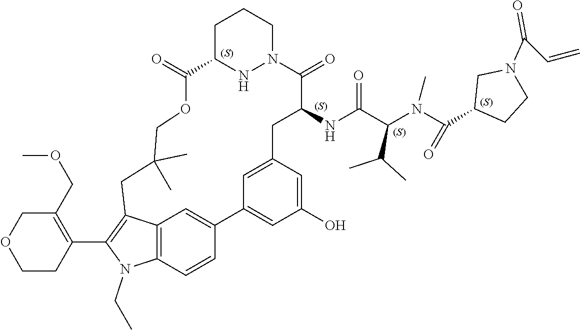 |
| BA59 | 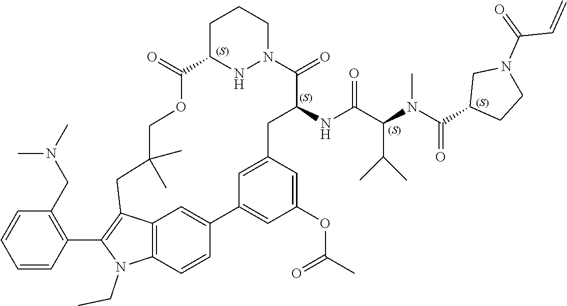 |
| BA60 | 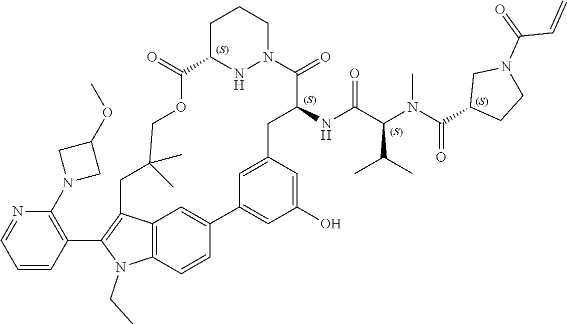 |
| BA61 | 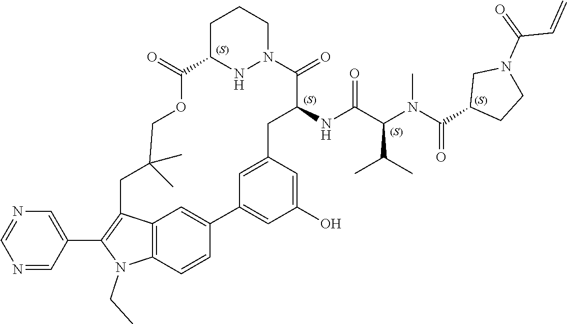 |
| BA62 | 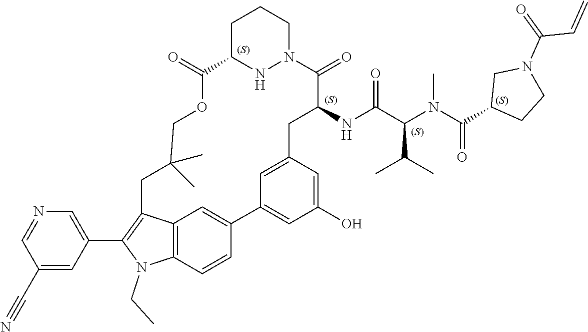 |
| BA63 | 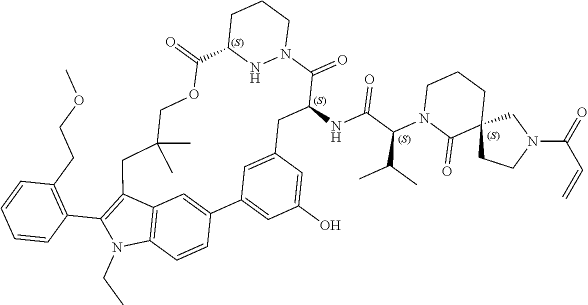 |
| BA64 | 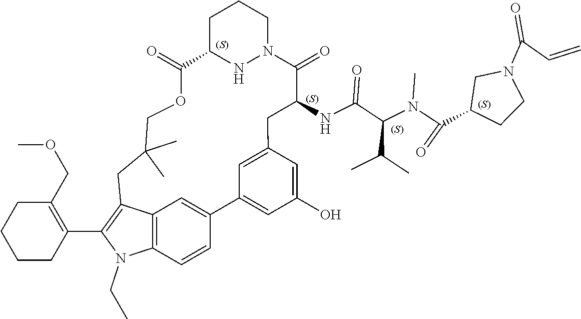 |
| BA65 | 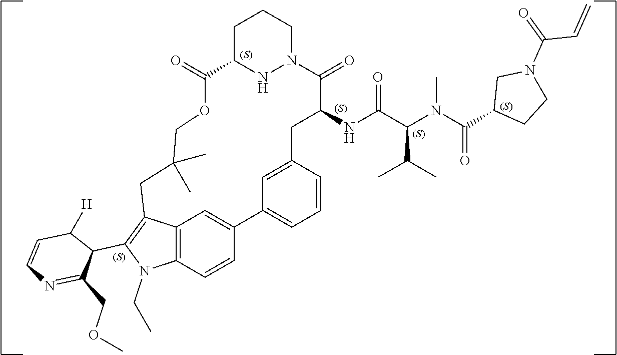 |
| BA66 | 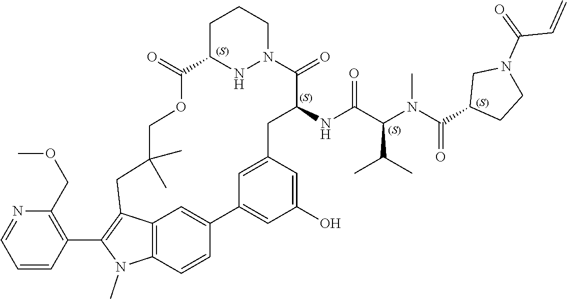 |
| BA67 | 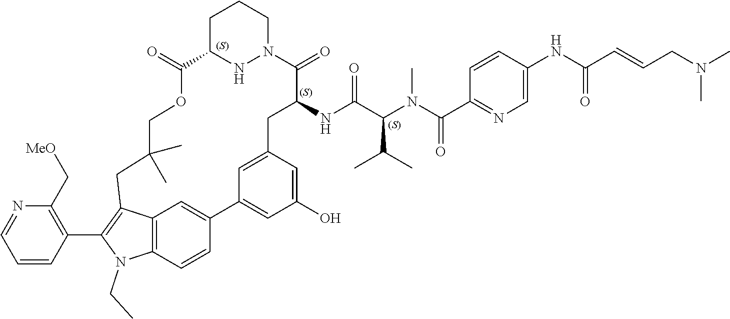 |
| BA68 | 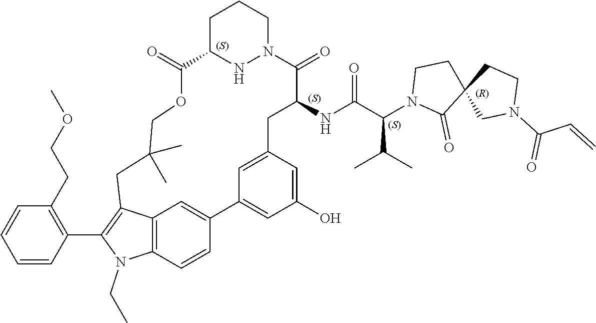 |
| BA69 | 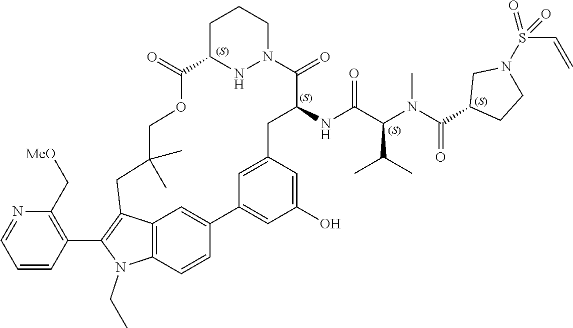 |
| BA70 | 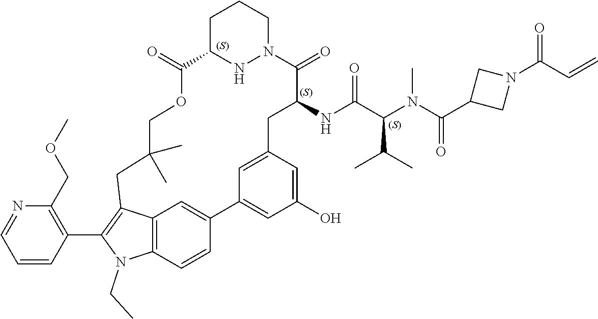 |
| BA71 | 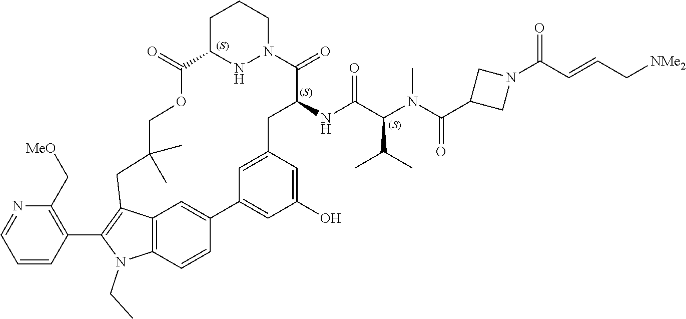 |
| BA72 | 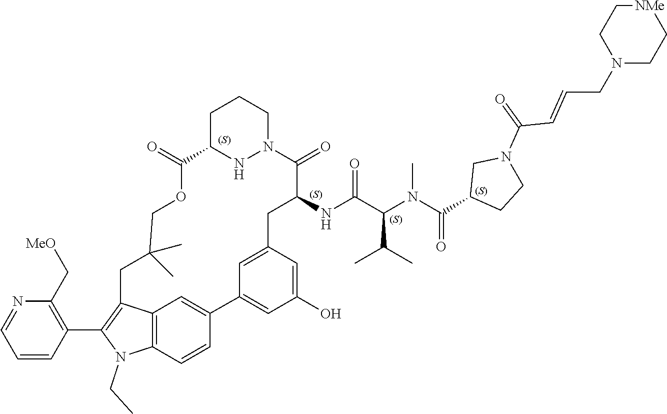 |
| BA73 | 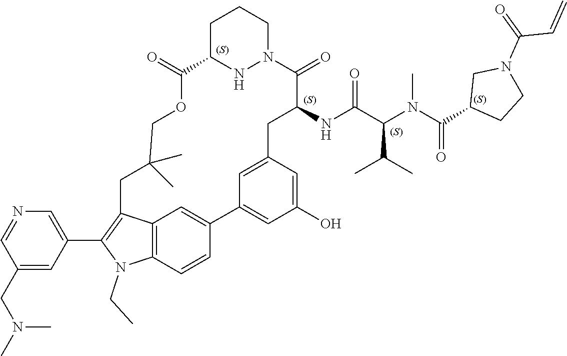 |
| BA74 | 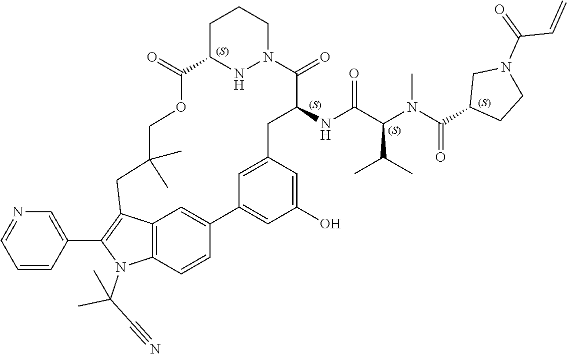 |
| BA75 | 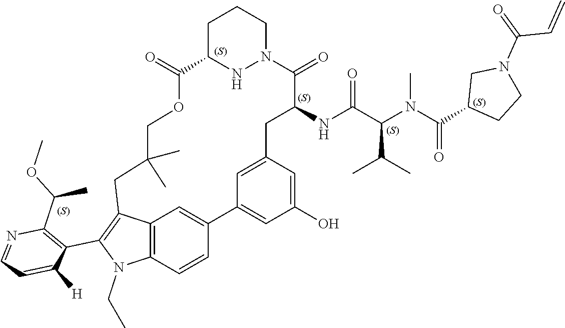 |
| BA76 | 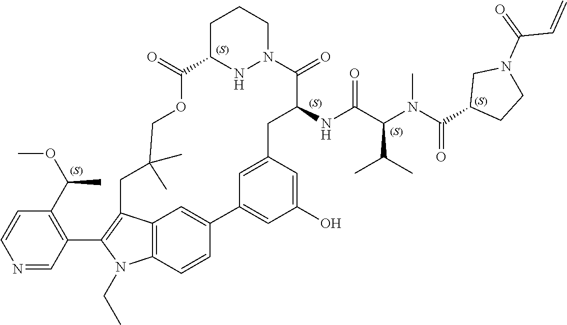 |
| BA77 | 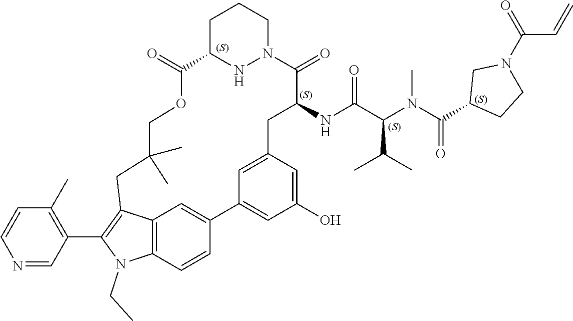 |
| BA78 | 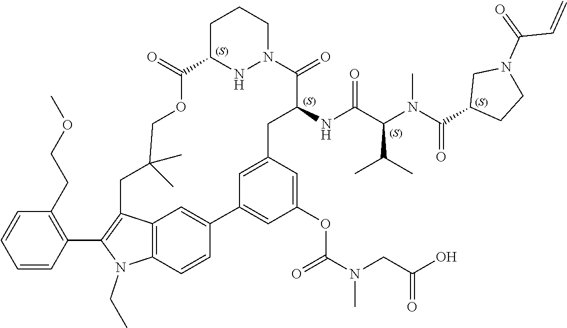 |
| BA79 | 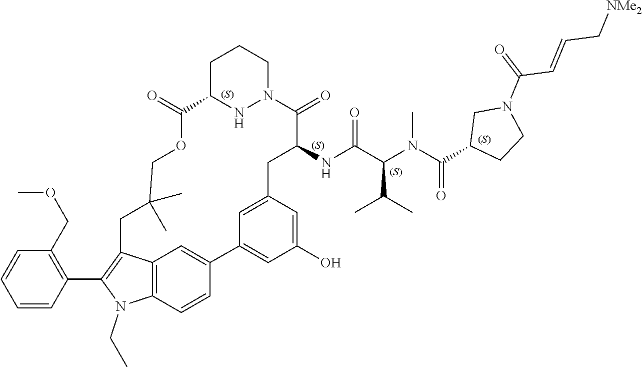 |
| BA80 | 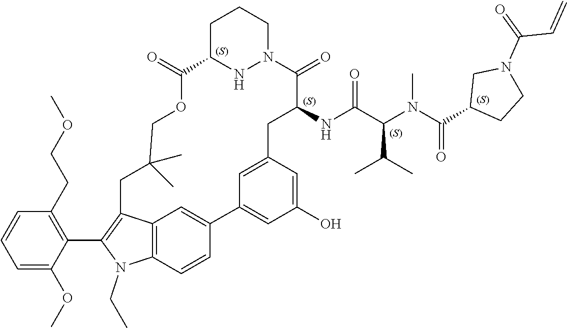 |
| BA81 | 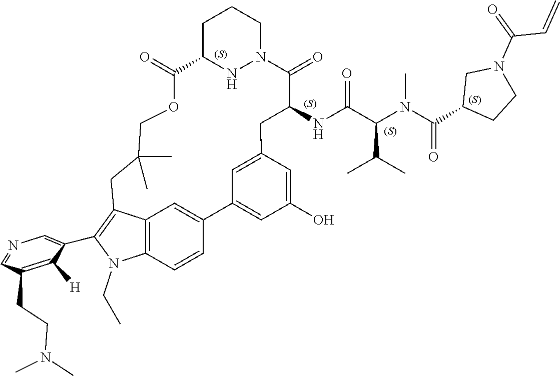 |
| BA82 | 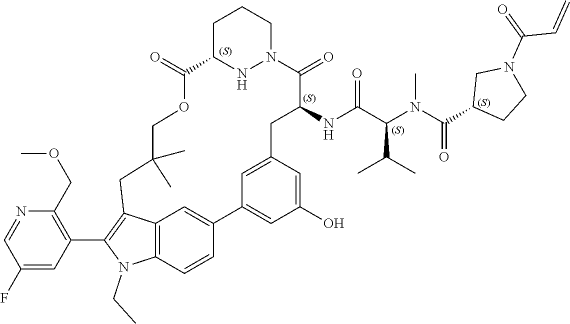 |
| BA83 | 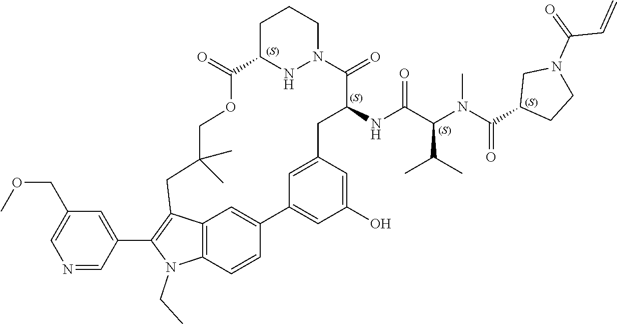 |
| BA84 | 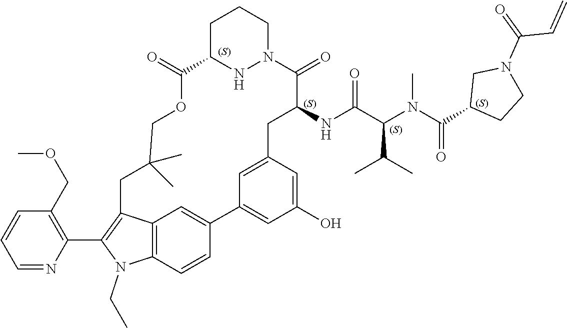 |
| BA85 | 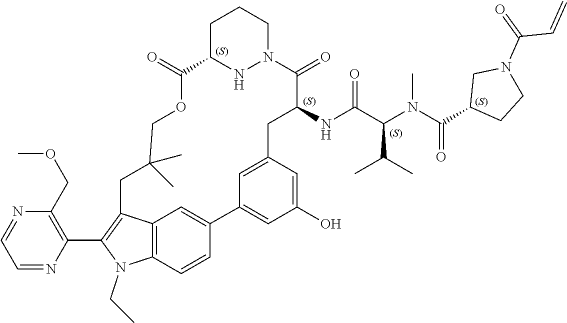 |
| BA86 | 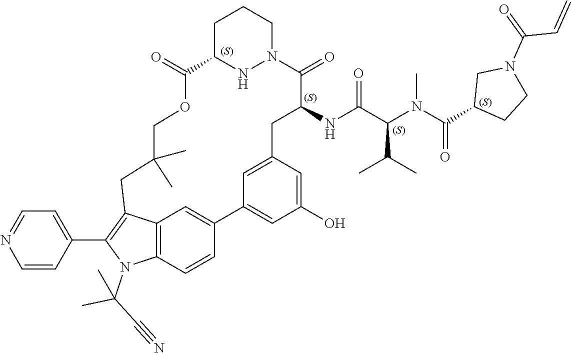 |
| BA87 | 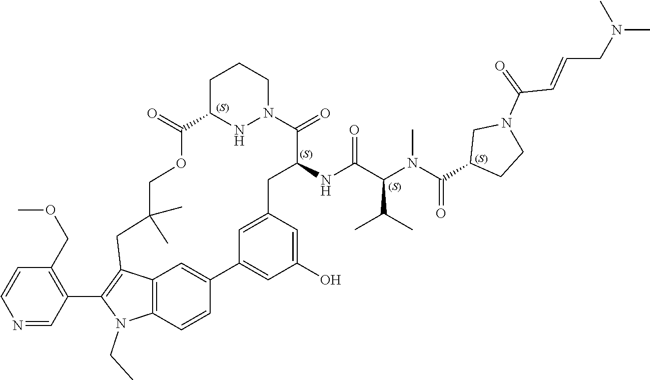 |
| BA88 | 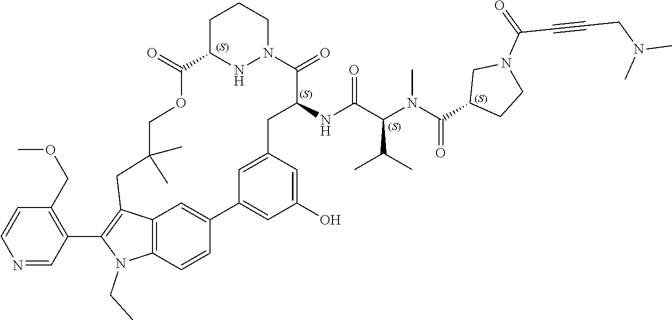 |
| BA89 | 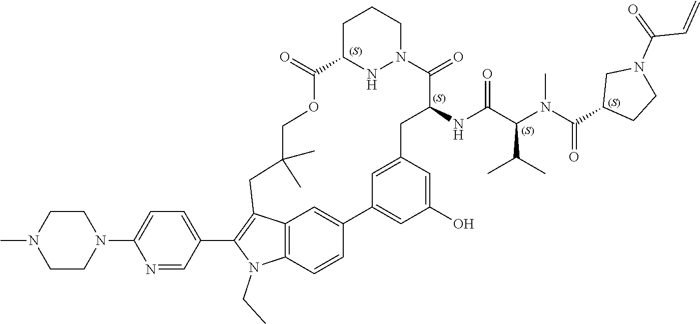 |
| BA90 | 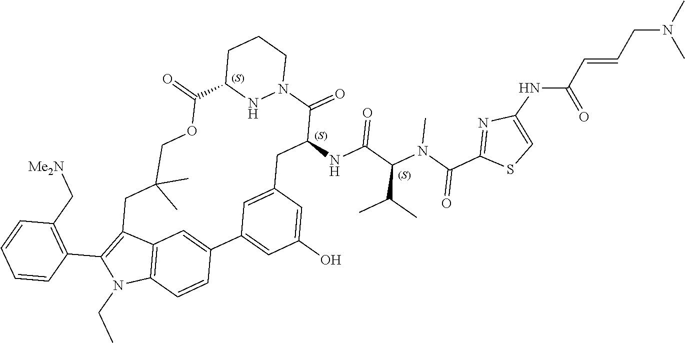 |
| BA91 | 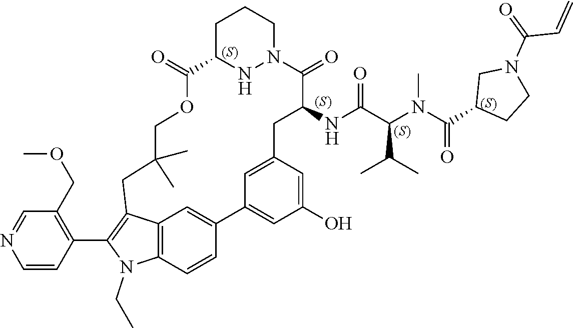 |
| BA92 | 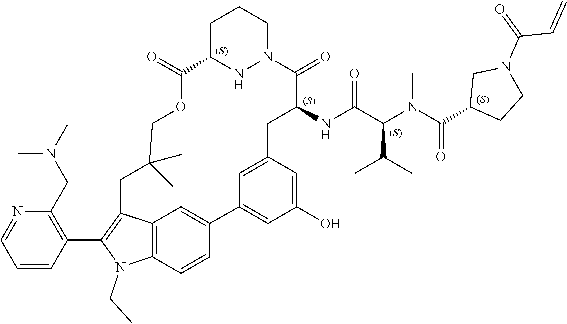 |
| BA93 | 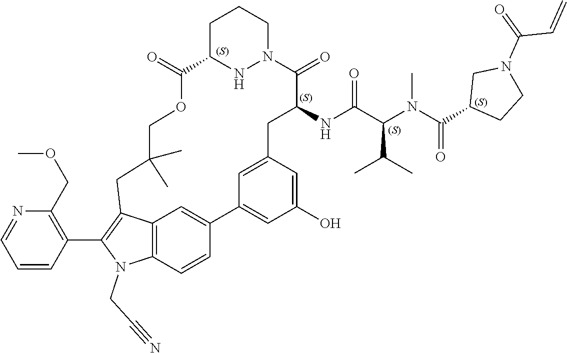 |
| BA94 | 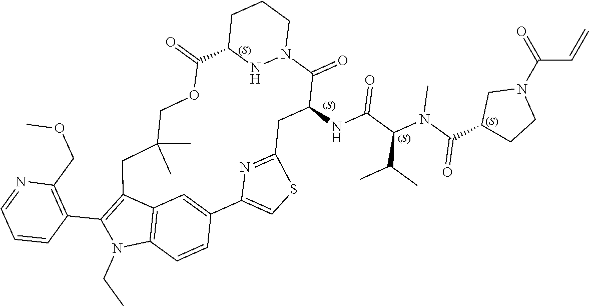 |
| BA95 | 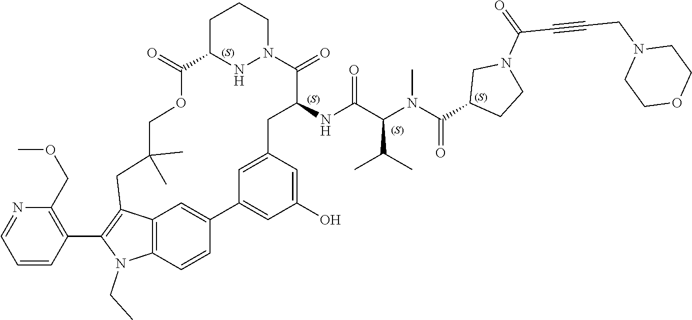 |
| BA96 | 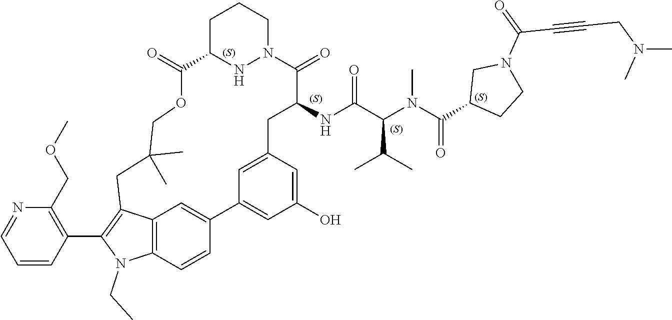 |
| BA97 | 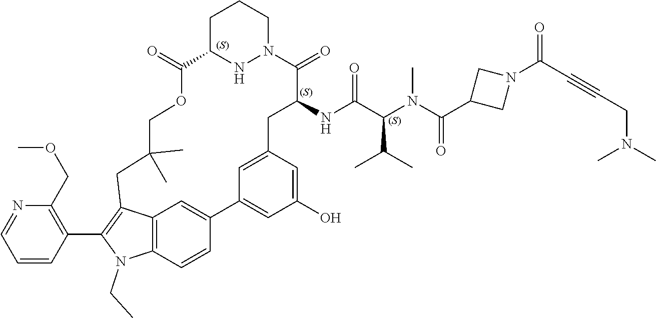 |
| BA98 | 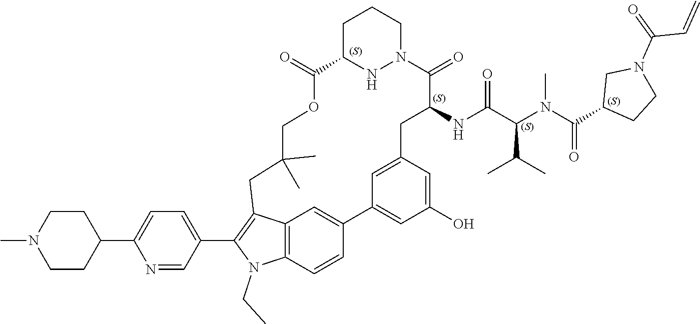 |
| BA99 | 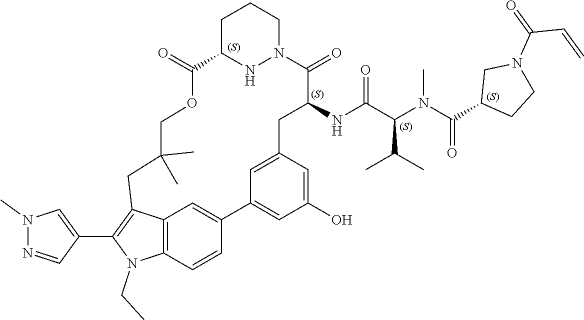 |
| BA100 | 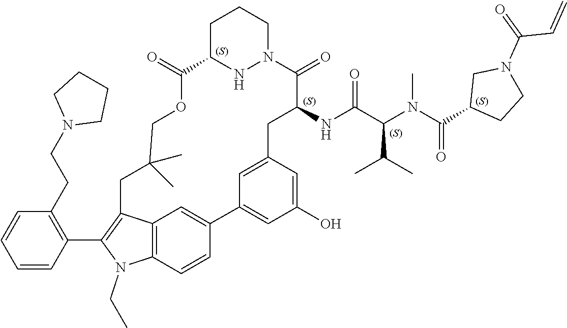 |
| BA101 | 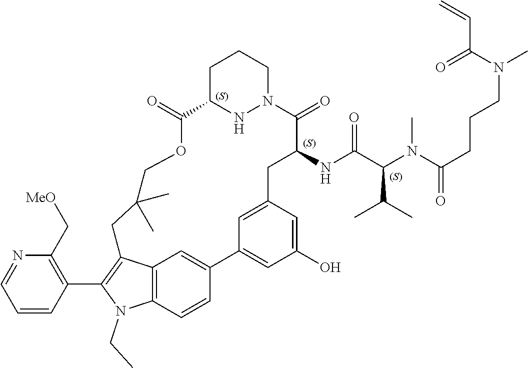 |
| BA102 | 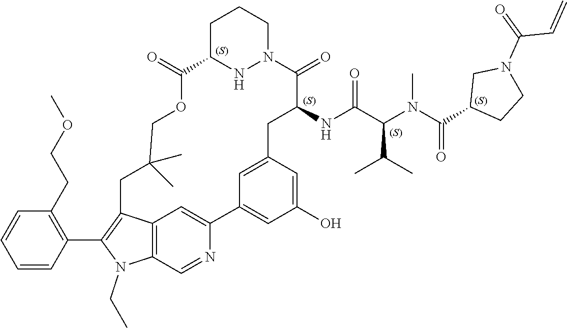 |
| BA103 | 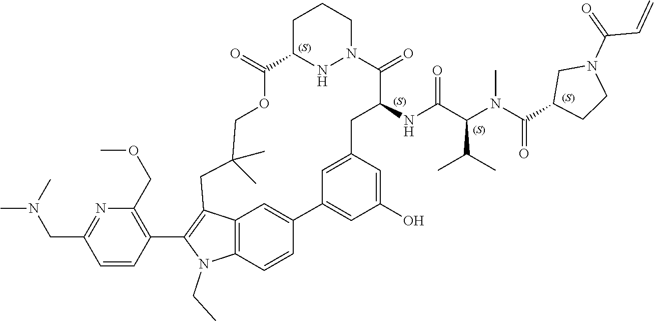 |
| BA104 | 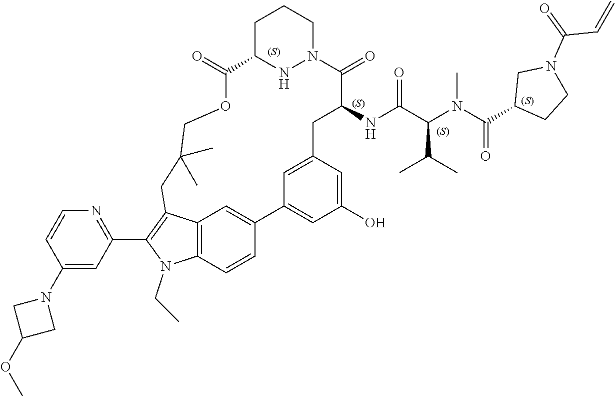 |
| BA105 | 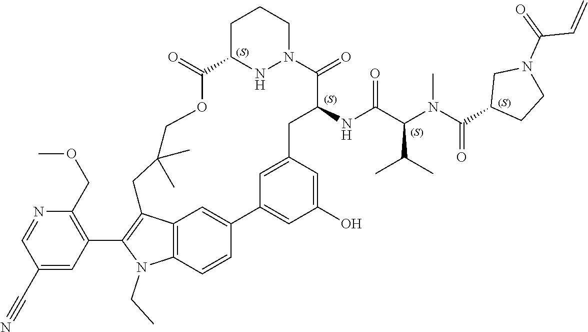 |
| BA106 | 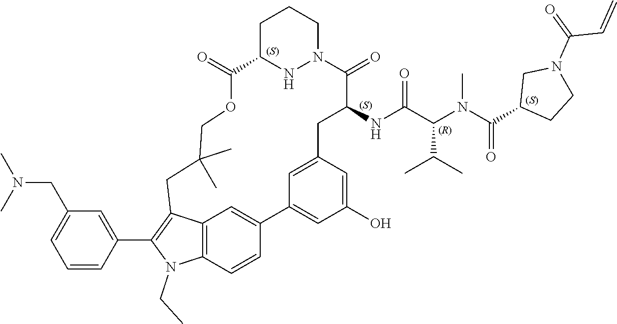 |
| BA107 | 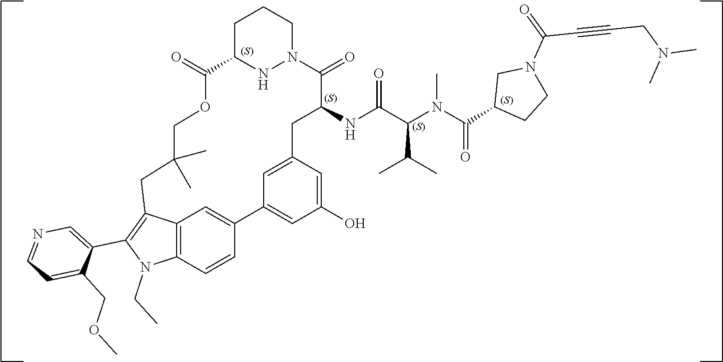 |
| BA108 | 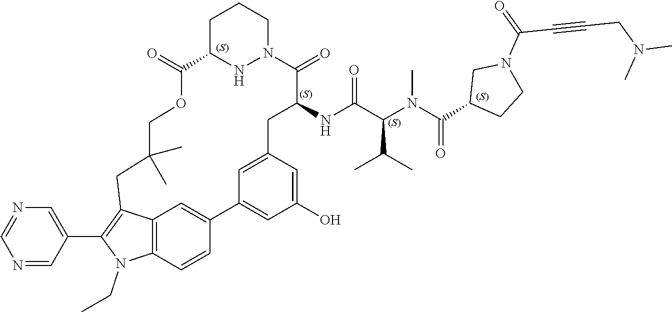 |
| BA109 | 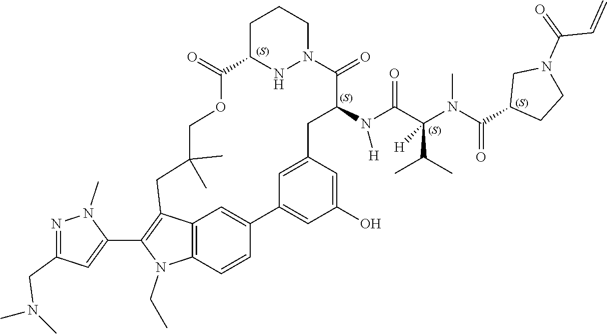 |
| BA110 | 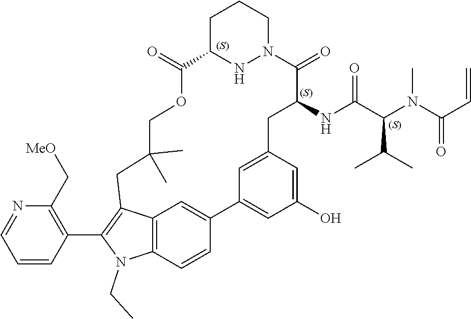 |
| BA111 | 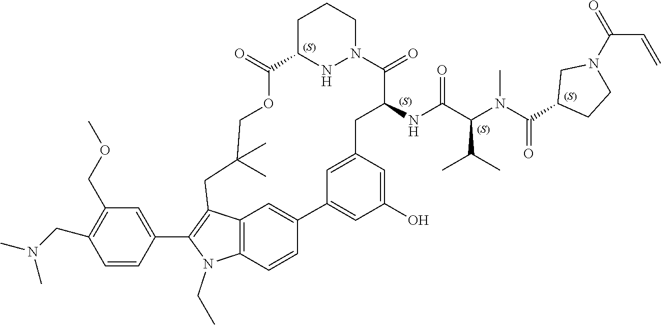 |
| BA112 | 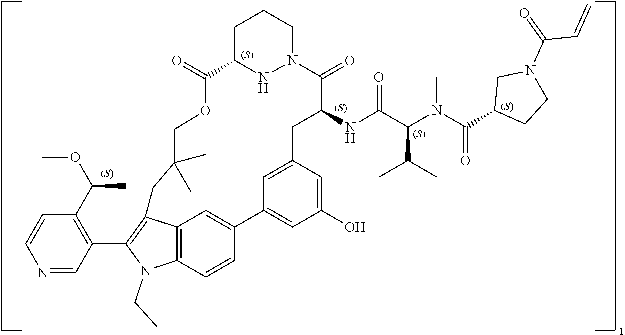 |
| BA113 | 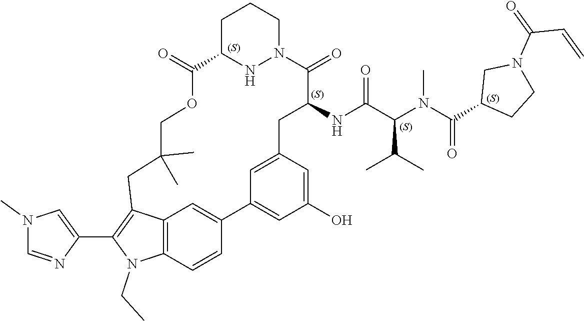 |
| BA114 | 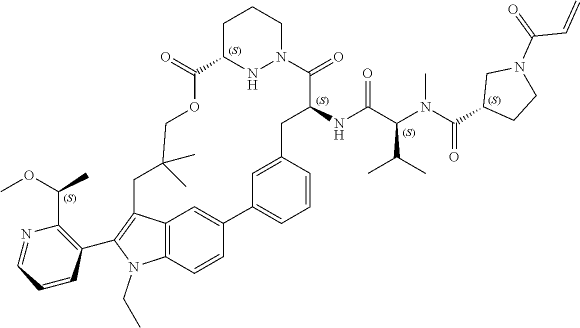 |
| BA115 | 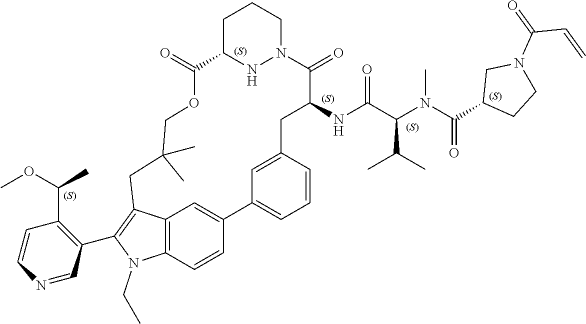 |
| BA116 | 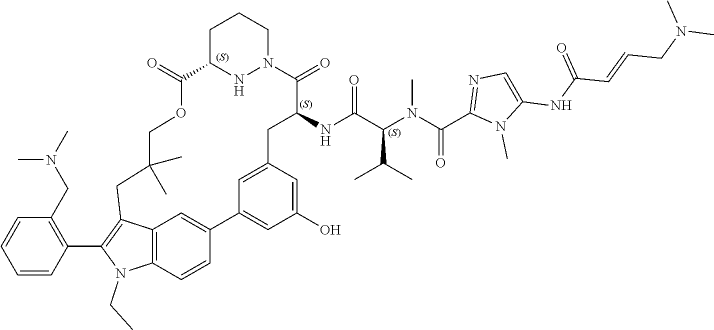 |
| BA117 | 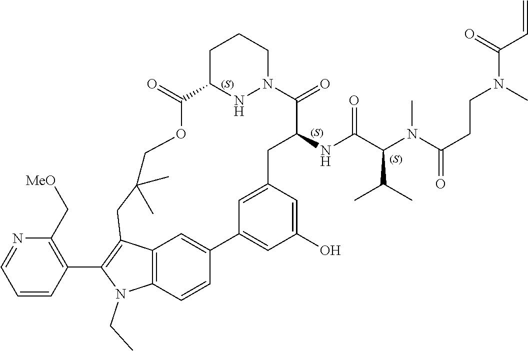 |
| BA118 | 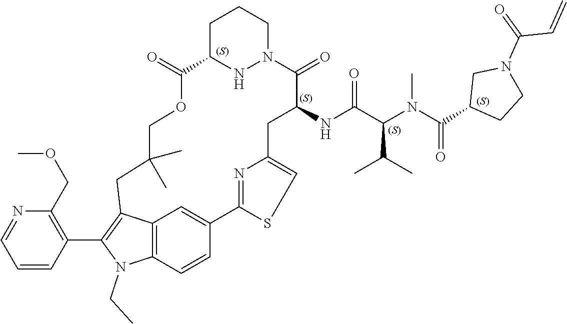 |
| BA119 | 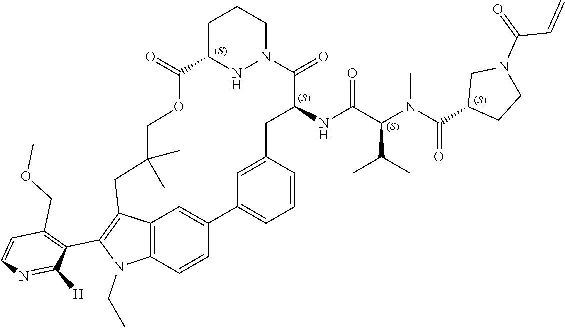 |
| BA120 | 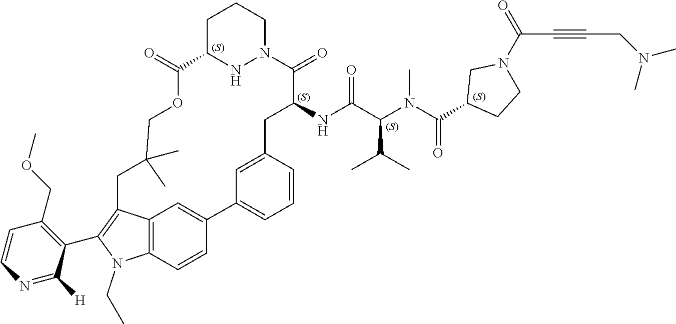 |
| BA121 | 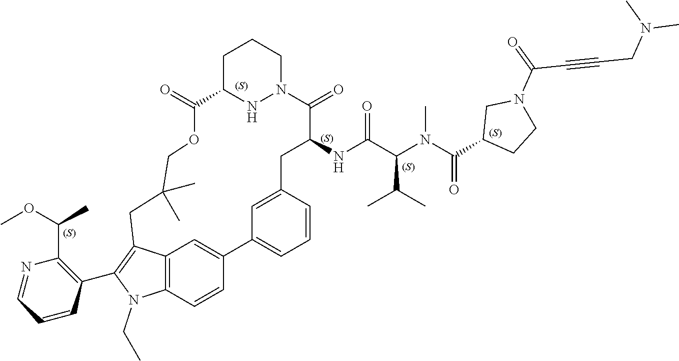 |
| BA122 | 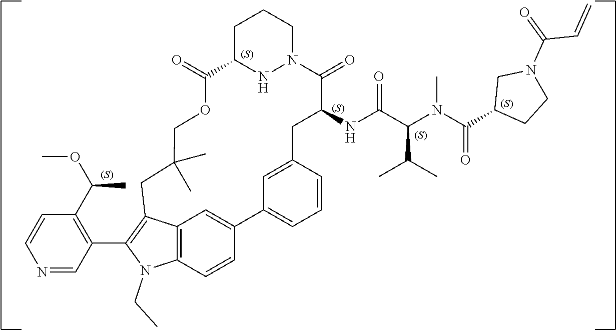 |
| BA123 | 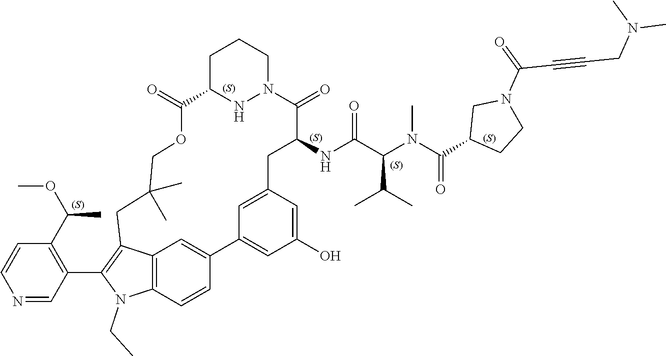 |
| BA124 | 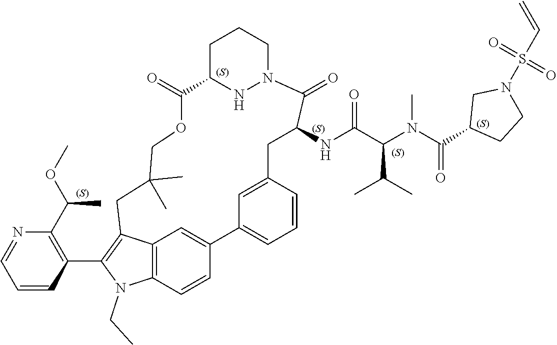 |
| BA125 | 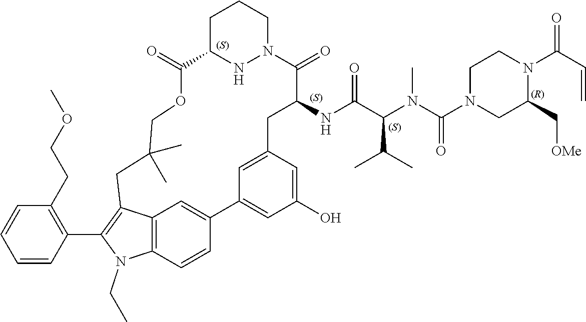 |
| BA126 | 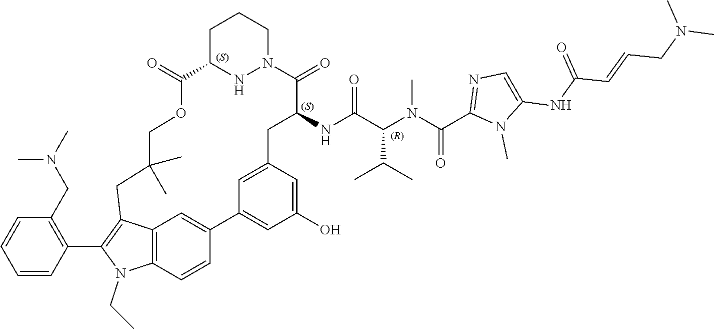 |
| BA127 | 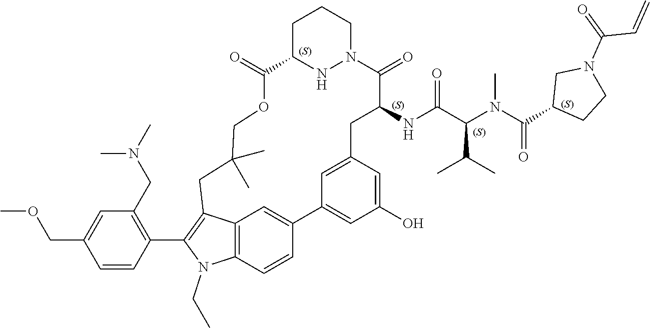 |
| BA128 | 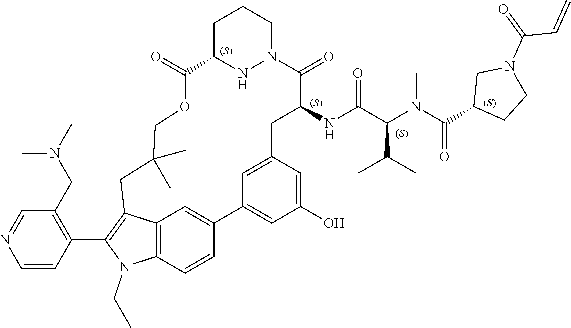 |
| BA129 | 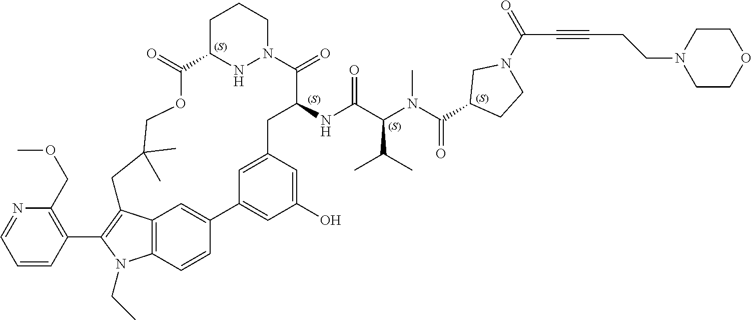 |
| BA130 | 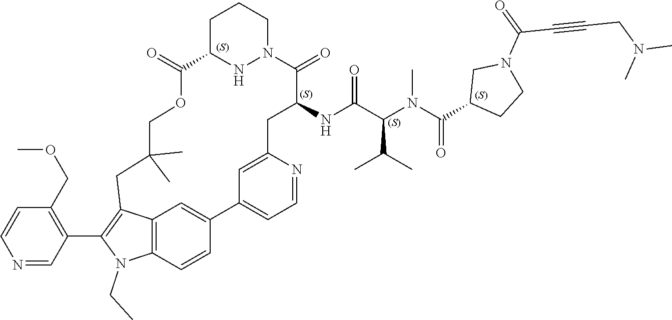 |
| BA131 | 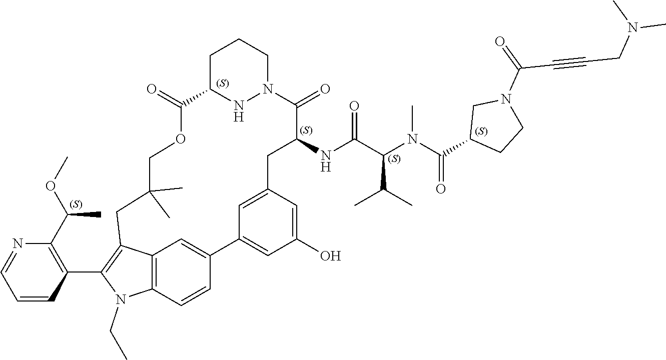 |
| BA132 | 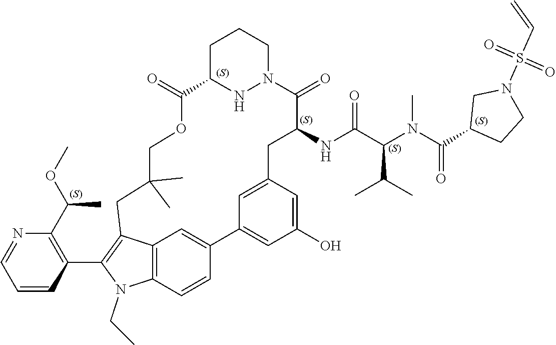 |
| BA133 | 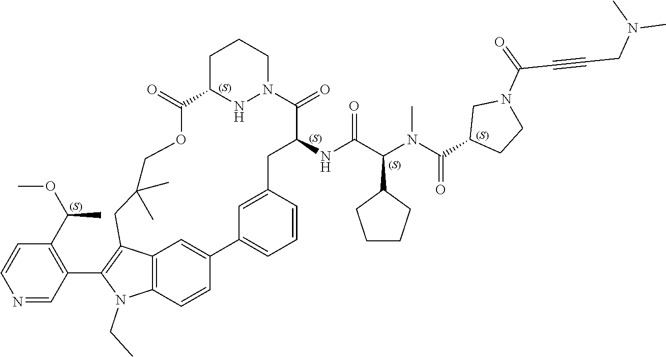 |
| BA134 | 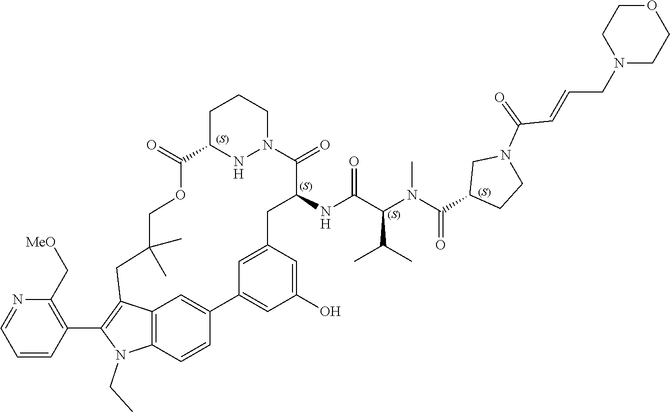 |
| BA135 | 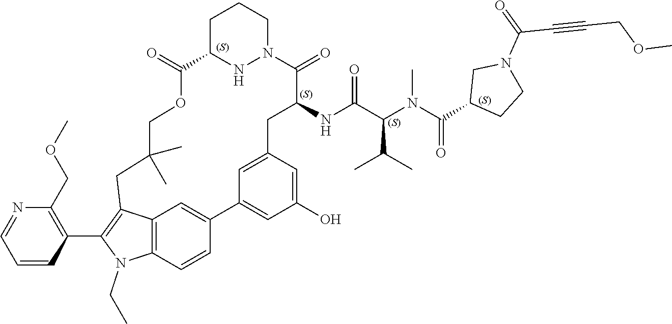 |
| BA136 | 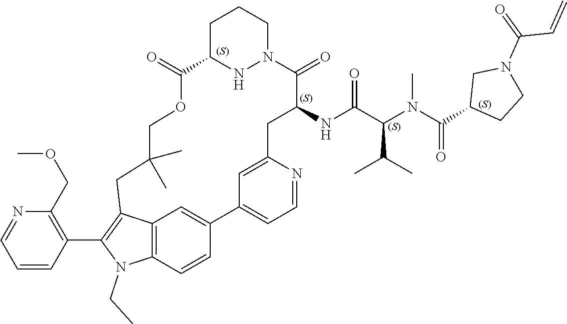 |
| BA137 | 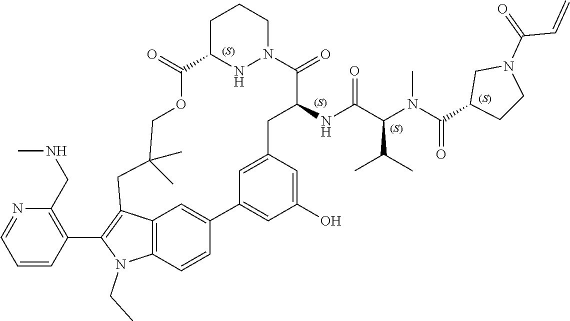 |
| BA138 | 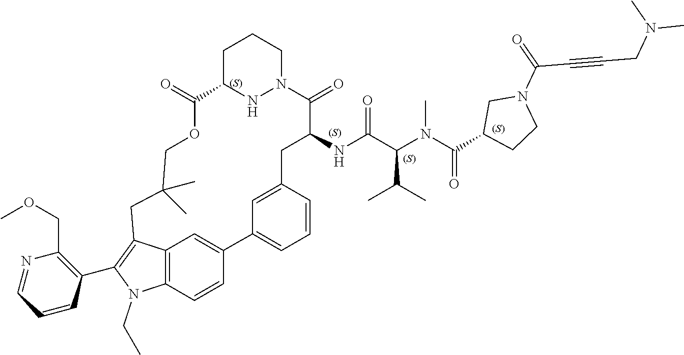 |
| BA139 | 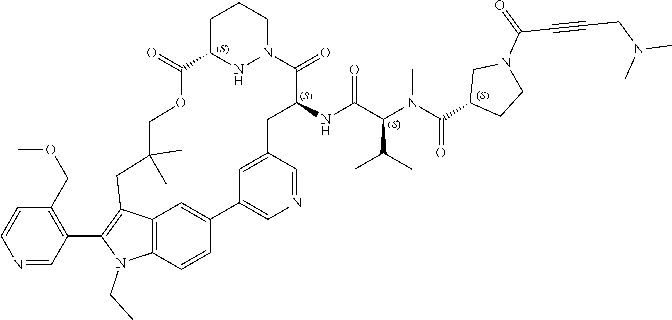 |
| BA140 | 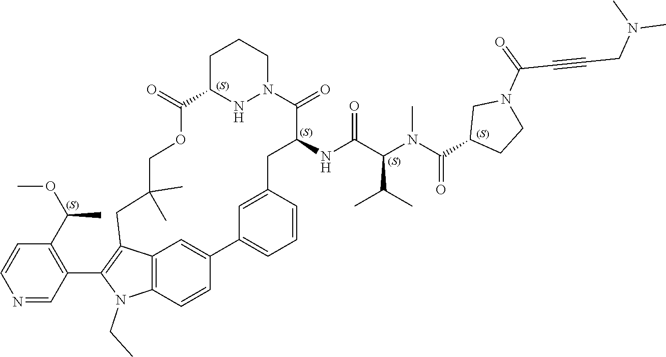 |
| BA141 | 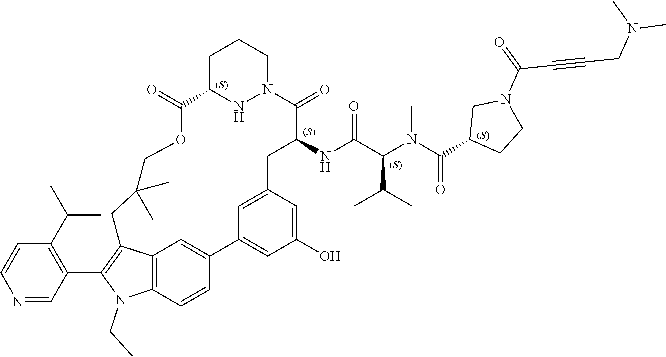 |
| BA142 | 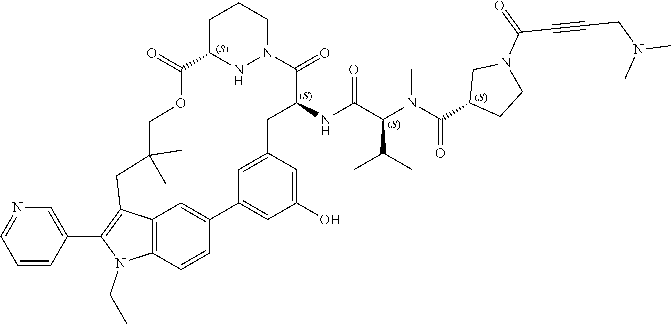 |
| BA143 | 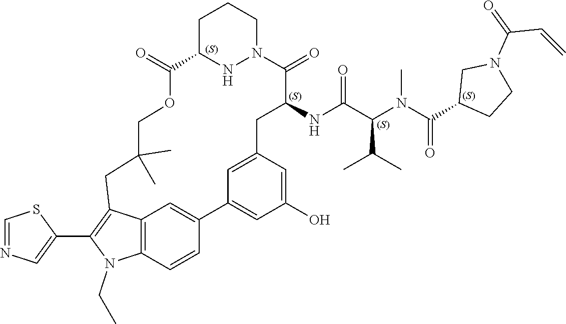 |
| BA144 | 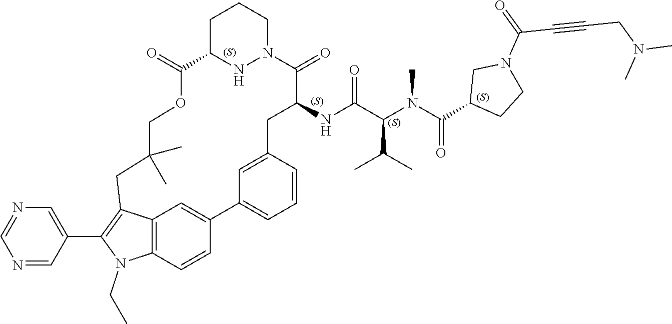 |
| BA145 | 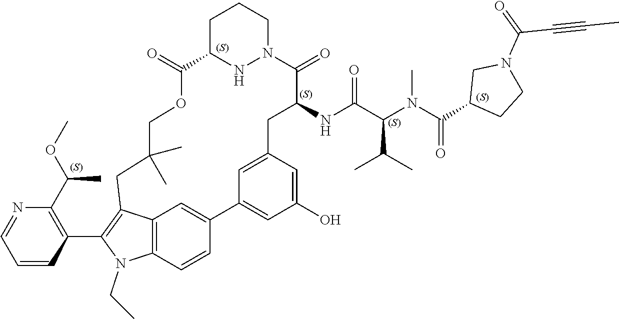 |
| BA146 | 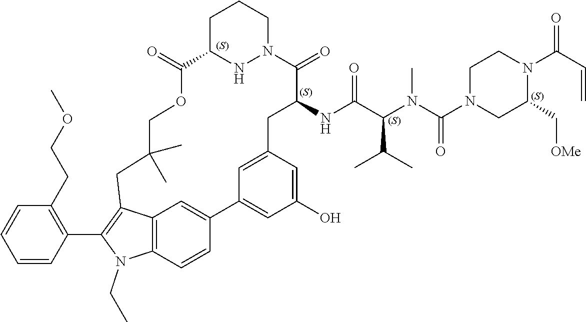 |
| BA147 | 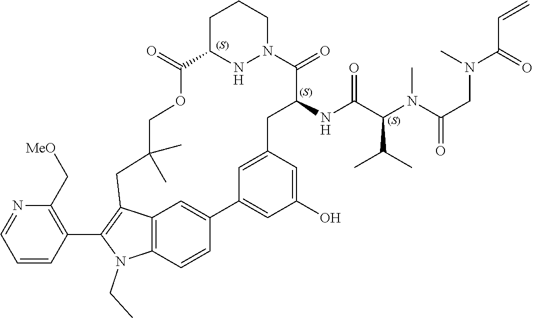 |
| BA148 | 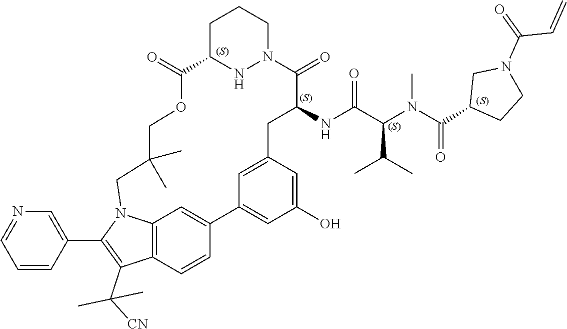 |
| BA149 | 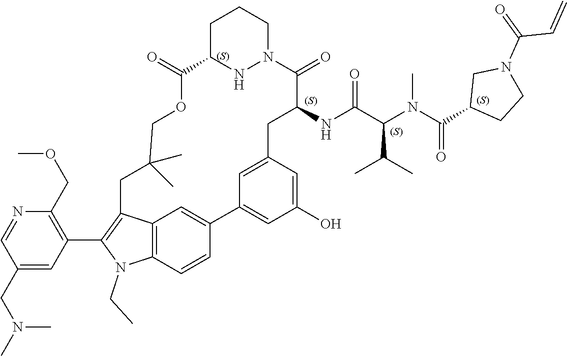 |
| BA150 | 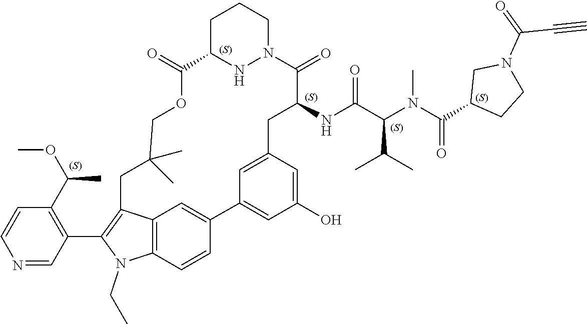 |
| BA151 | 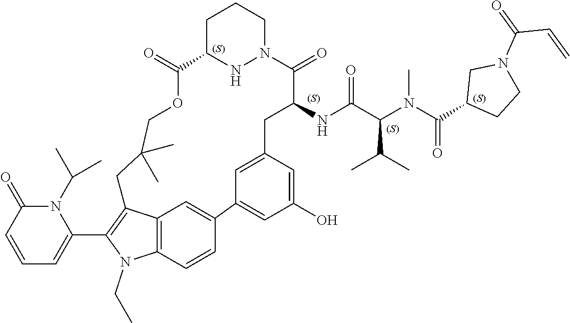 |
| BA152 | 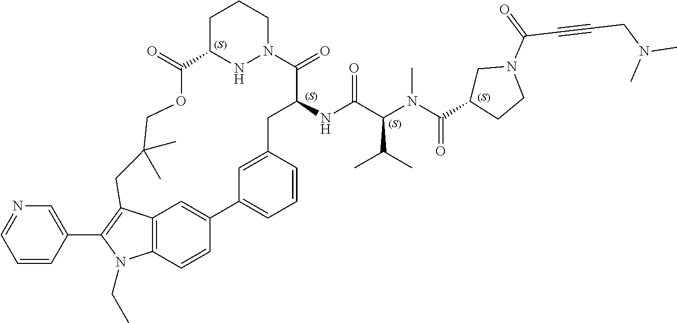 |
| BA153 | 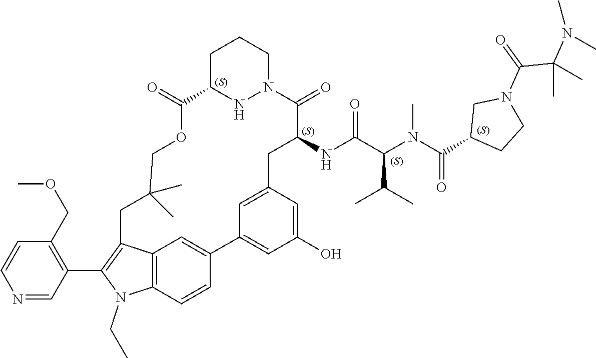 |
| BA154 | 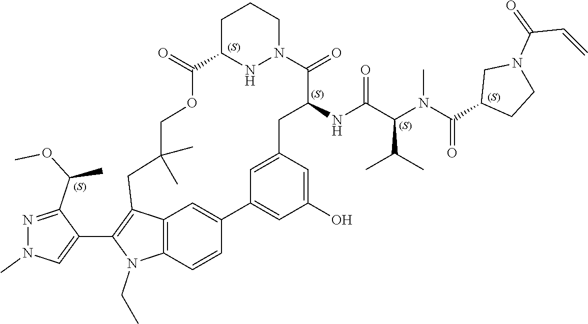 |
| BA155 | 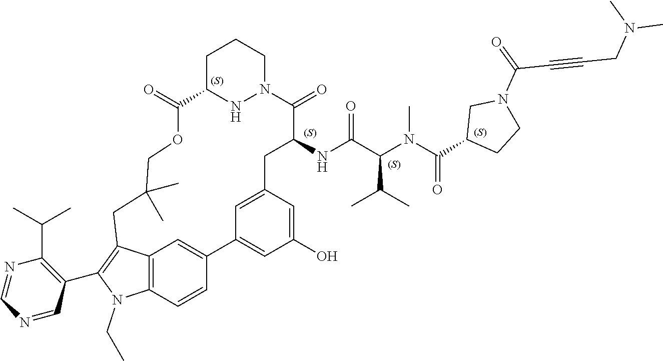 |
| BA156 | 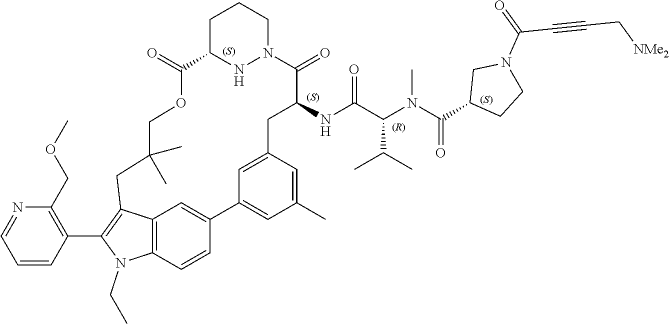 |
| BA157 | 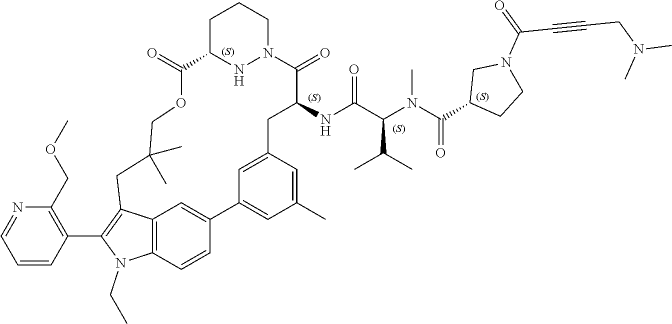 |
| BA158 | 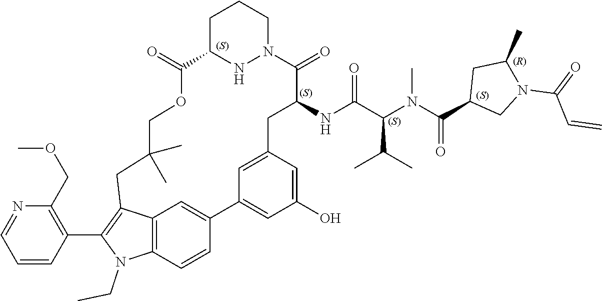 |
| BA159 | 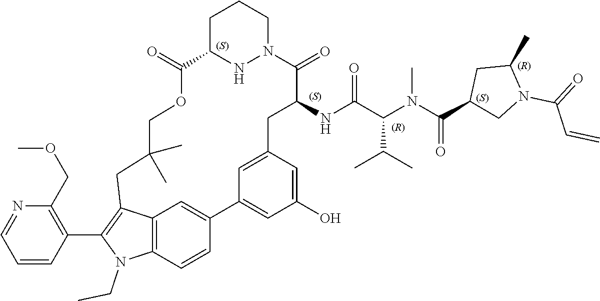 |
| BA160 | 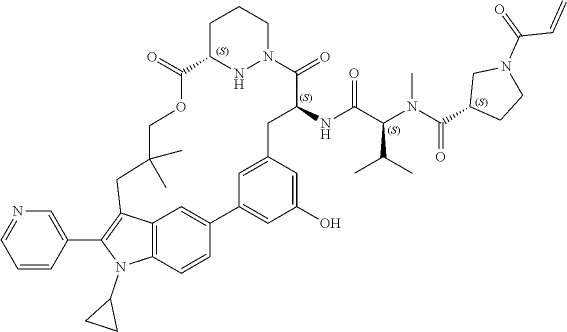 |
| BA161 | 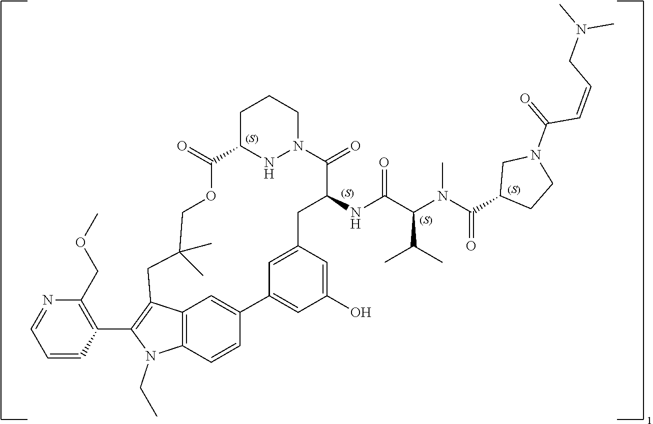 |
| BA162 | 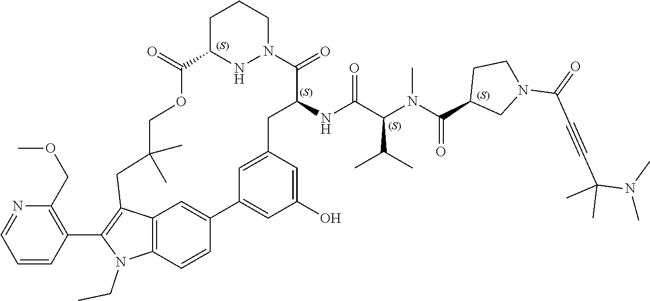 |
| BA163 | 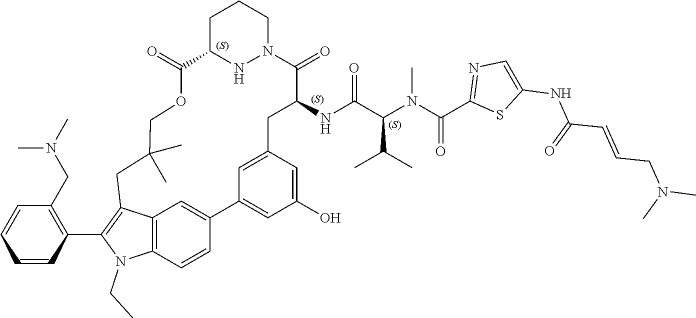 |
| BA164 | 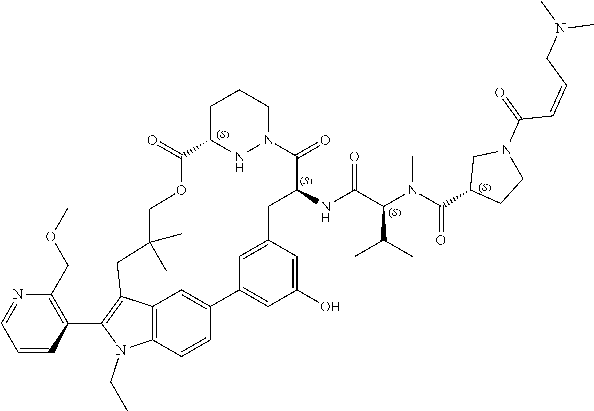 |
| BA165 | 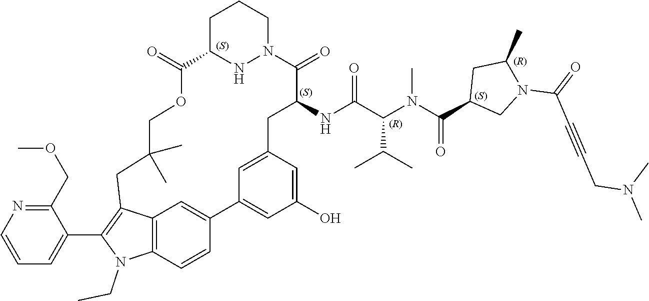 |
| BA166 | 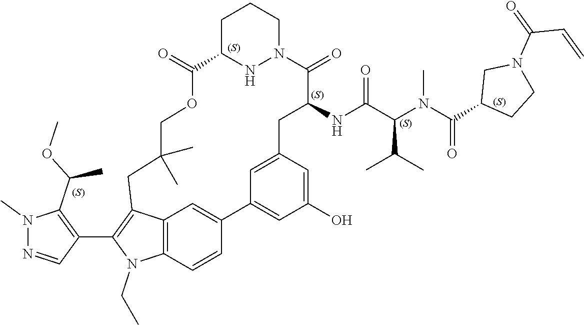 |
| BA167 | 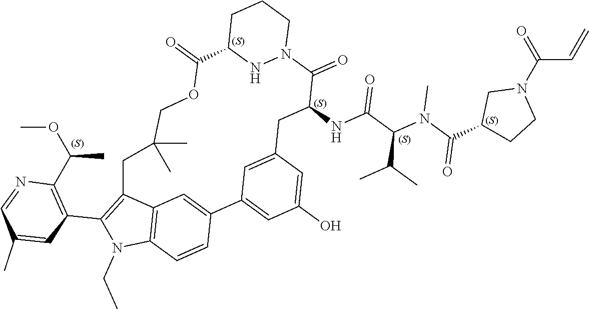 |
| BA168 | 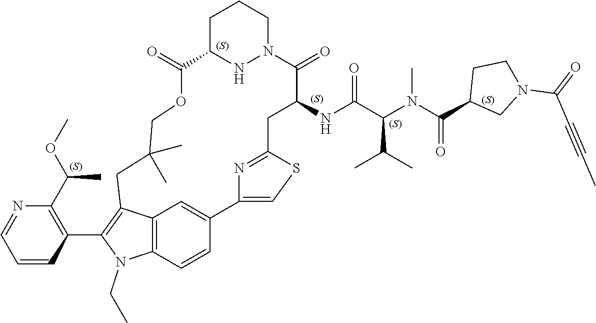 |
| BA169 | 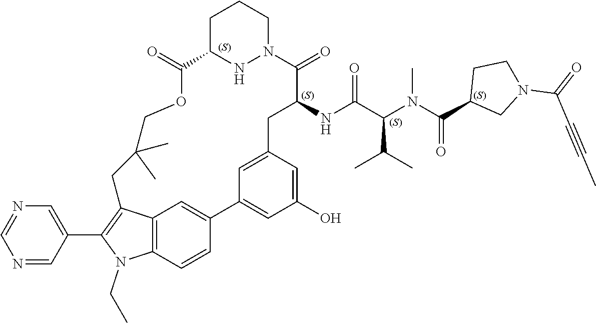 |
| BA170 | 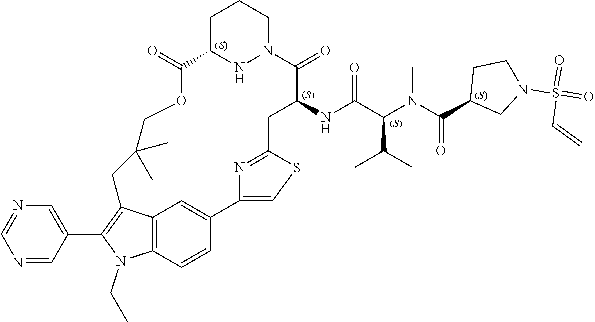 |
| BA171 | 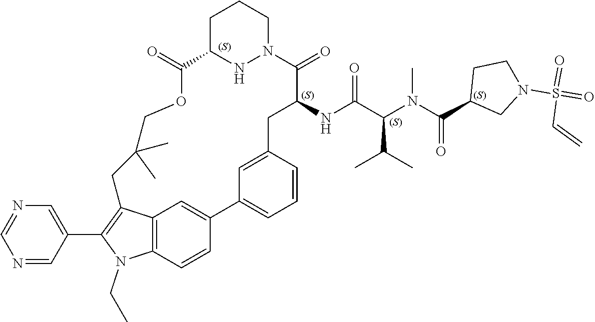 |
| BA172 | 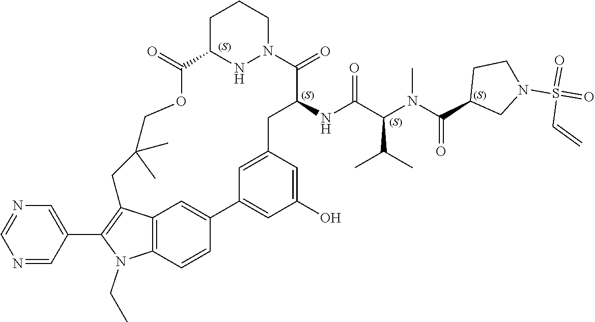 |
| BA173 | 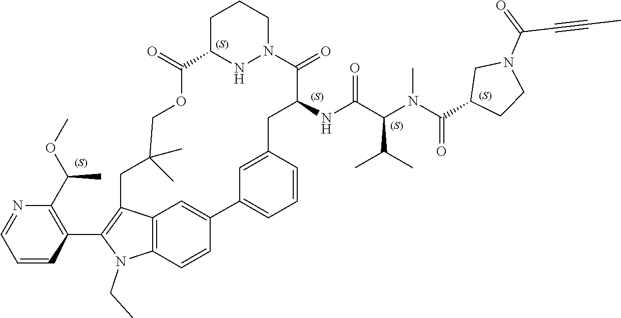 |
| BA174 | 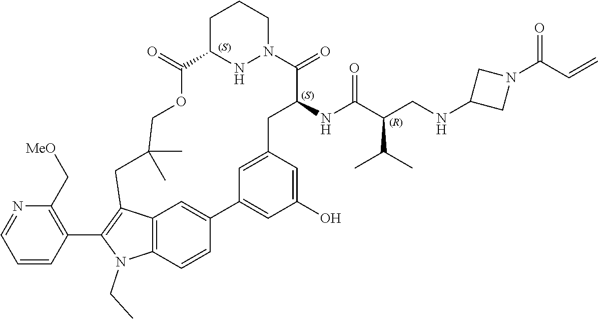 |
| BA175 | 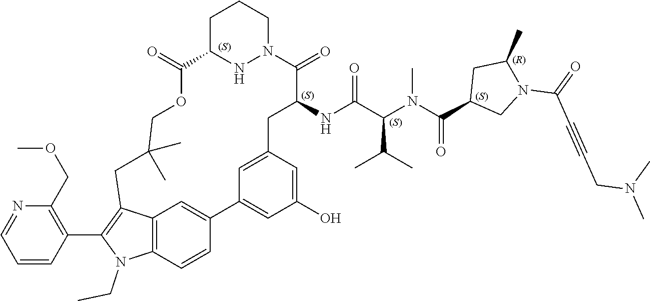 |
| BA176 | 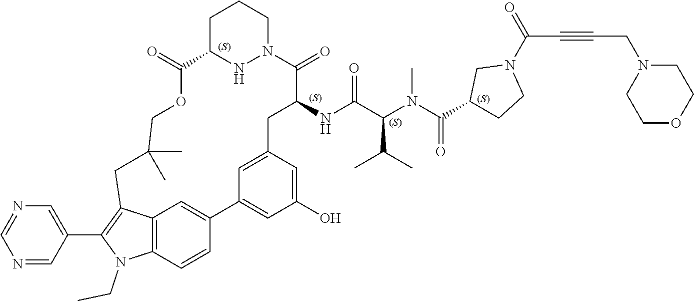 |
| BA177 | 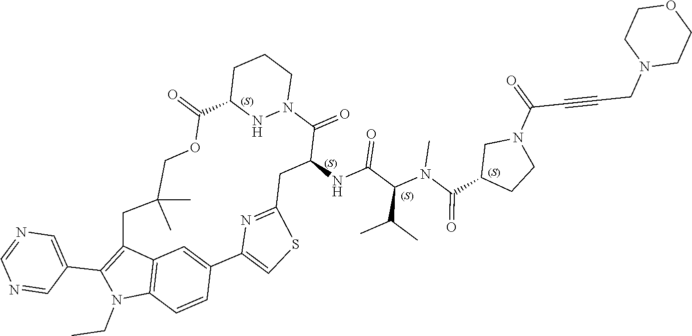 |
| BA178 | 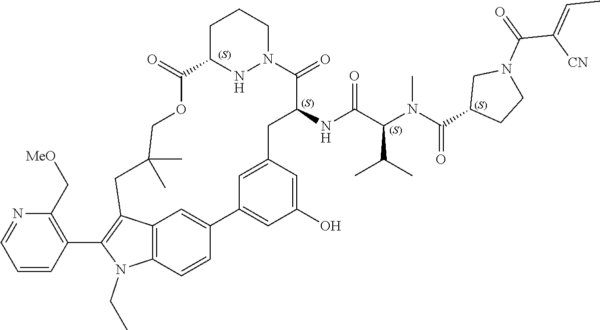 |
| BA179 | 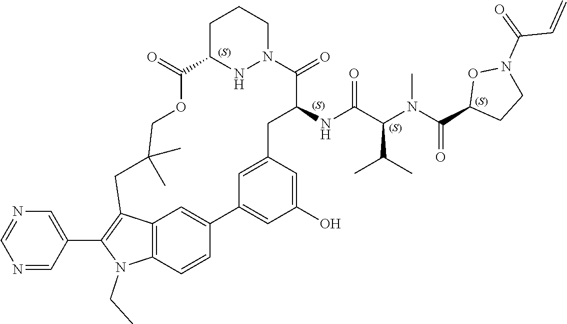 |
| BA180 | 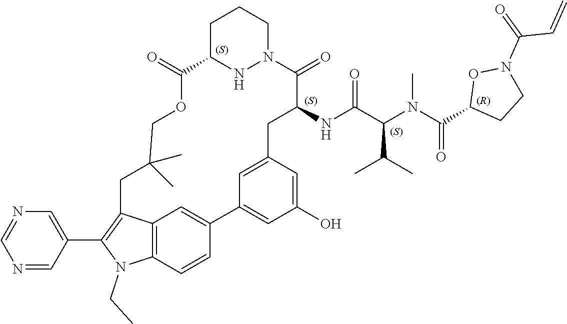 |
| BA181 | 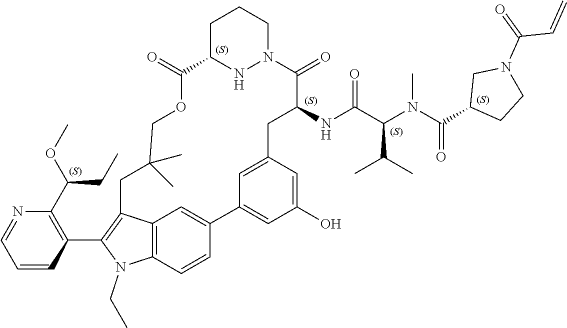 |
| BA182 | 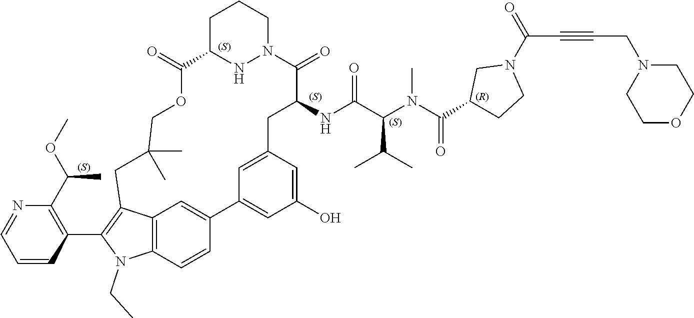 |
| BA183 | 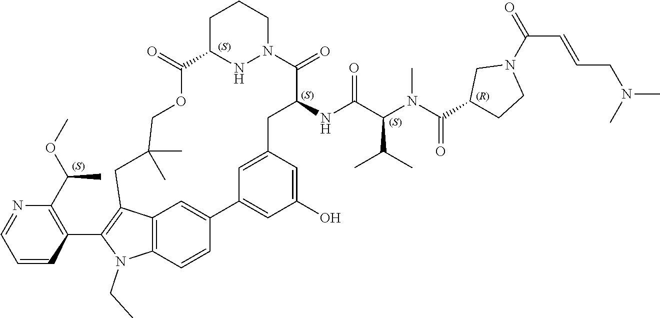 |
| BA184 | 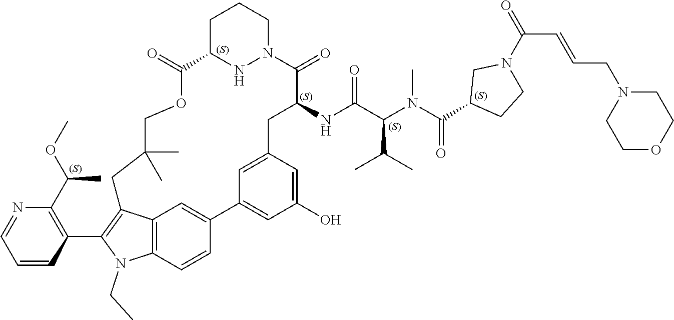 |
| BA185 | 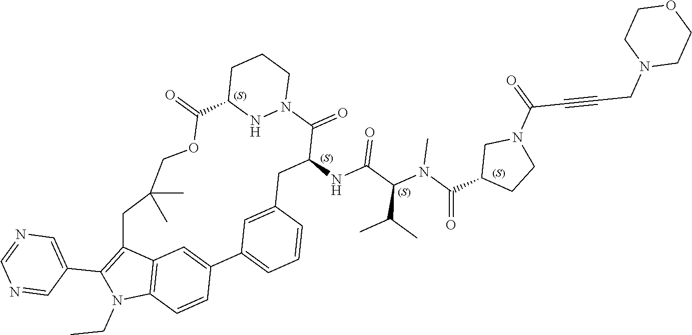 |
| BA186 | 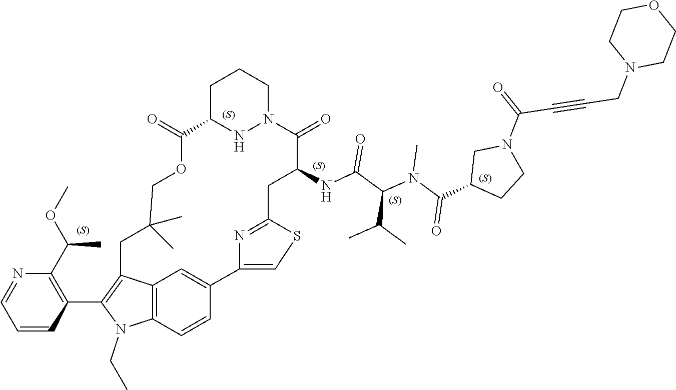 |
| BA187 | 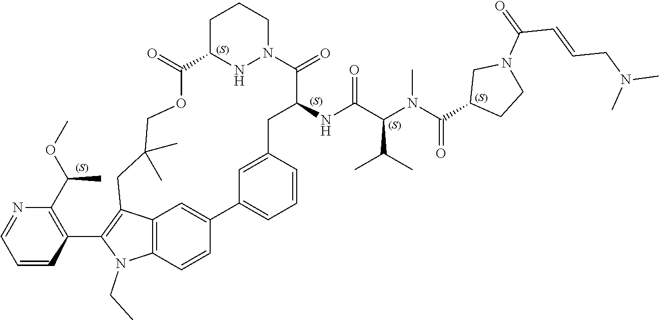 |
| BA188 | 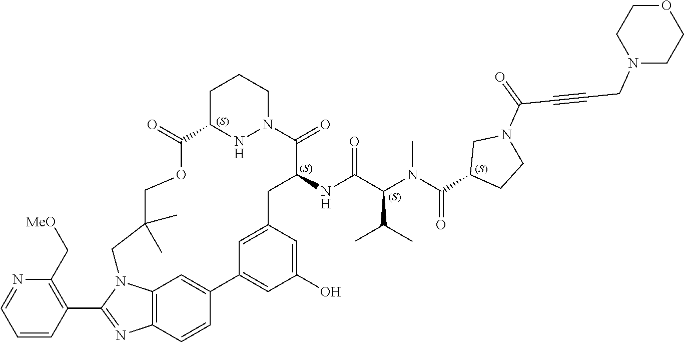 |
| BA189 | 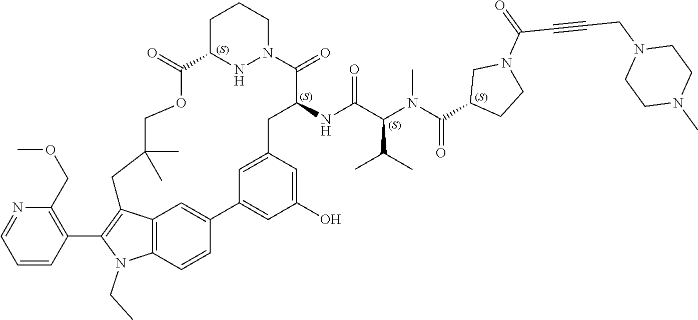 |
| BA190 | 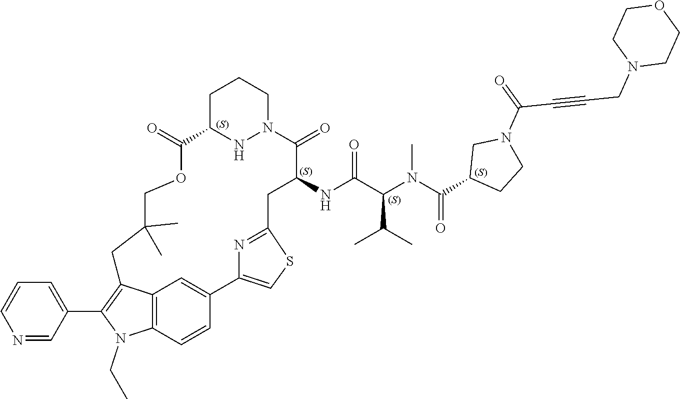 |
| BA191 | 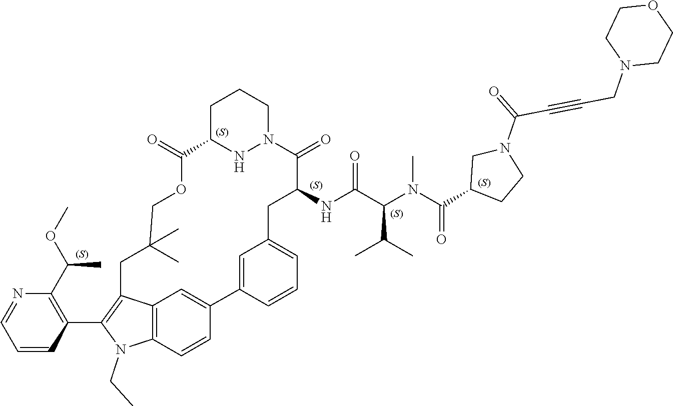 |
| BA192 | 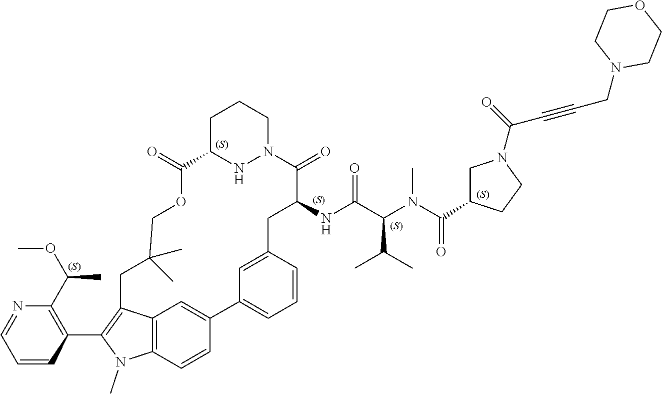 |
| BA193 | 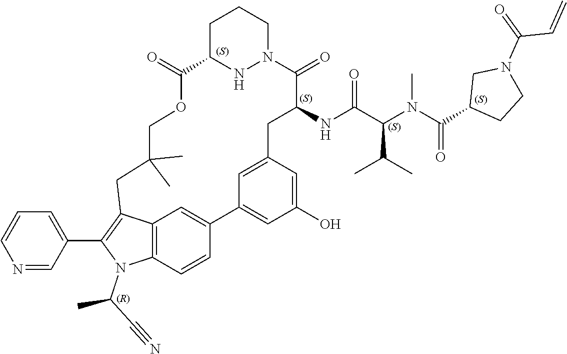 |
| BA194 | 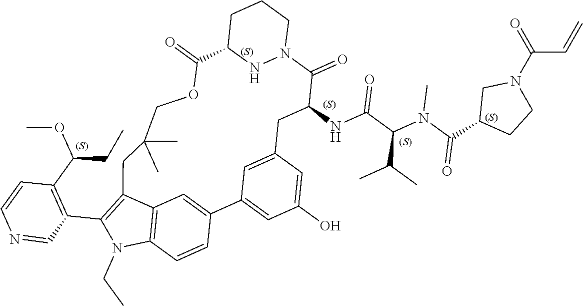 |
| BA195 | 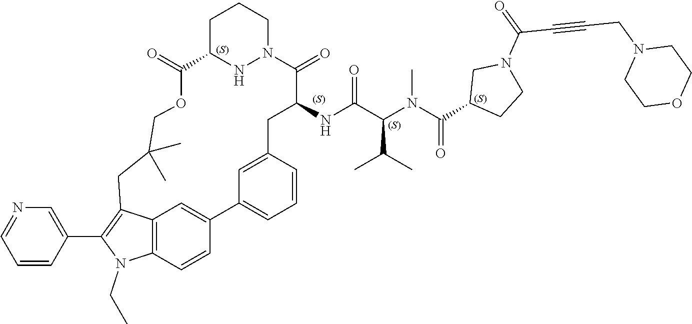 |
| BA196 | 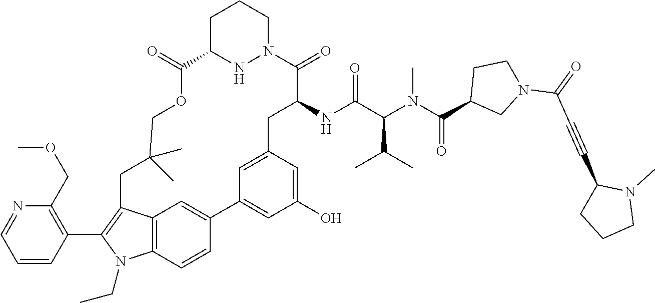 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@@H]3CCCN3C)C1)C(=O)N1CCC[C@H](N1)C(=O)OCC(C)(C)C2] |
| BA197 | 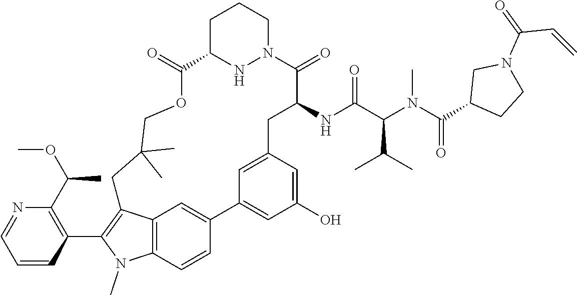 |
| BA198 | 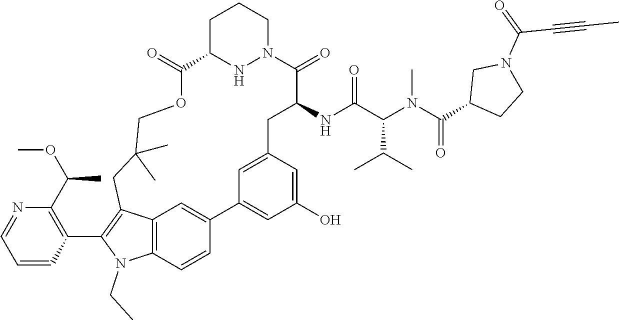 |
| BA199 | 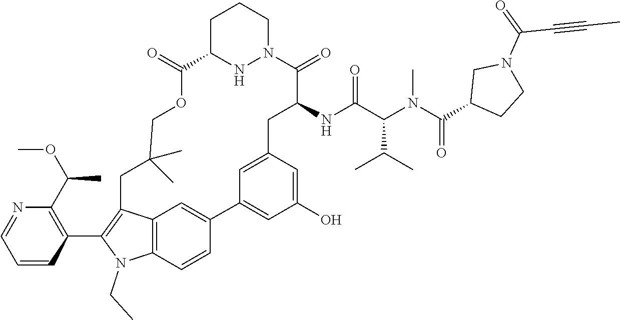 |
| BA200 | 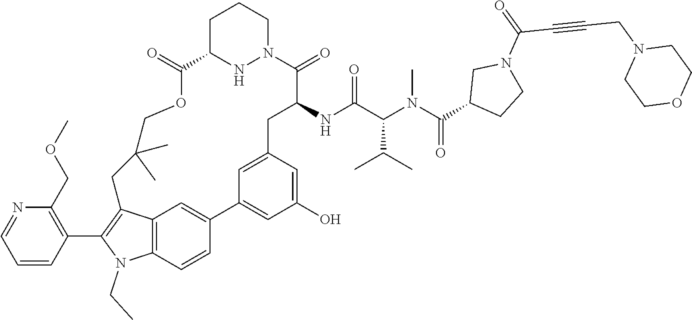 [SMILES: CCn1c(-c2cccnc2COC)c2c3cc(ccc31)-c1cc(O)cc(c1)C[C@H](NC(=O)[C@@H](C(C)C)N(C)C(=O)[C@H]1CCN(C(=O)C#CCN3CCOCC3)C1)C(=O)N1CCC[C@H](N1)C(=O)OCC(C)(C)C2] |
| BA201 | 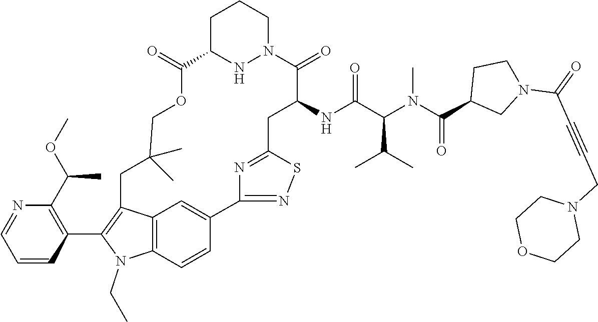 |
| BA202 | 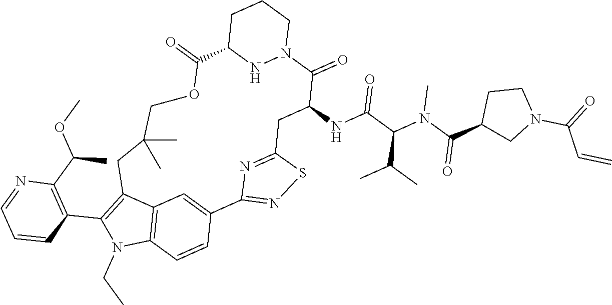 |
| BA203 | 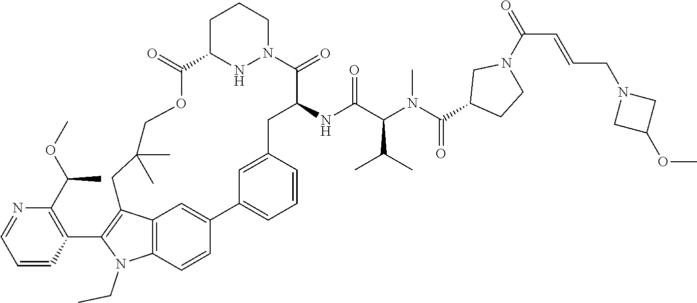 |
| BA204 | 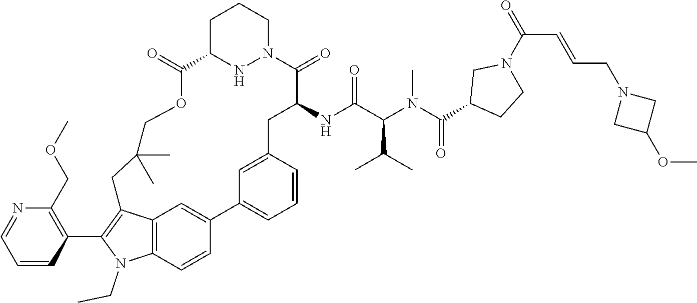 |
| BA205 | 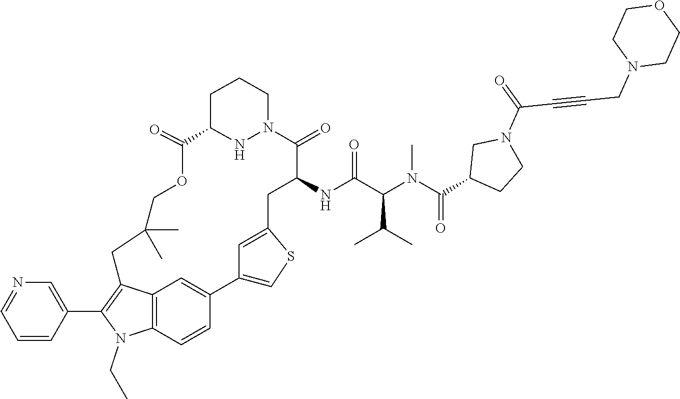 [SMILES: CCn1c(-c2cccnc2)c2c3cc(ccc31)-c1csc(c1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BA206 | 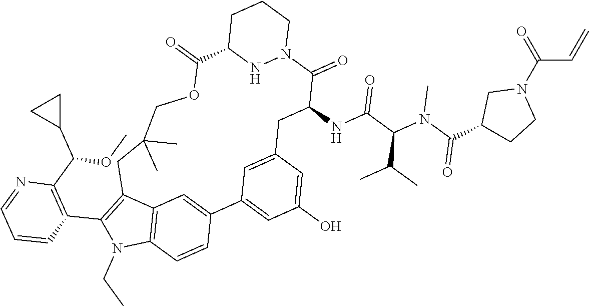 |
| BA207 | 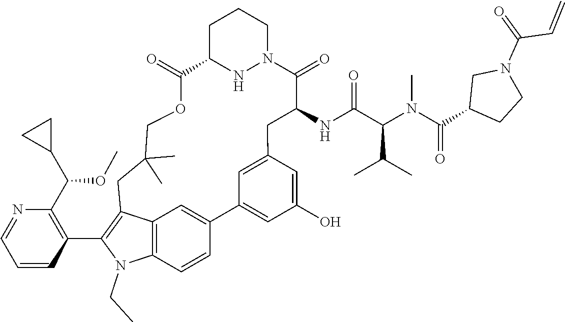 |
| BA208 | 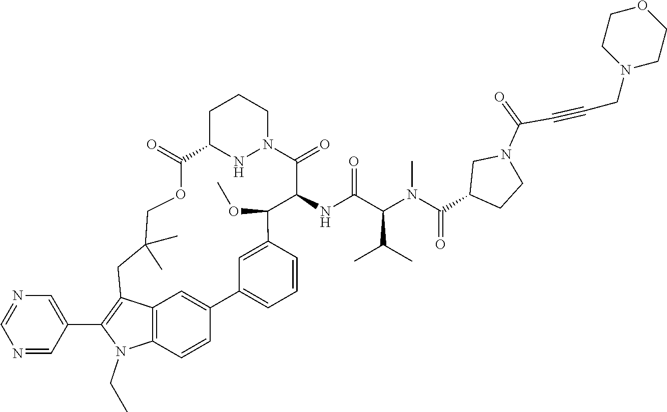 [SMILES: CCn1c(-c2cncnc2)c2c3cc(ccc31)-c1cccc(c1)[C@@H](OC)[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BA209 | 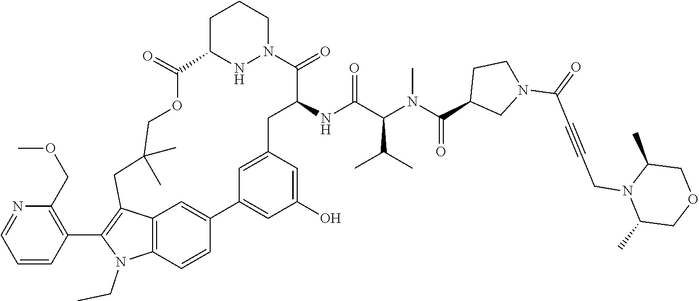 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CCN3[C@@H](C)COC[C@@H]3C)C1)C(=O)N1CCC[C@H](N1)C(=O)OCC(C)(C)C2] |
| BA210 | 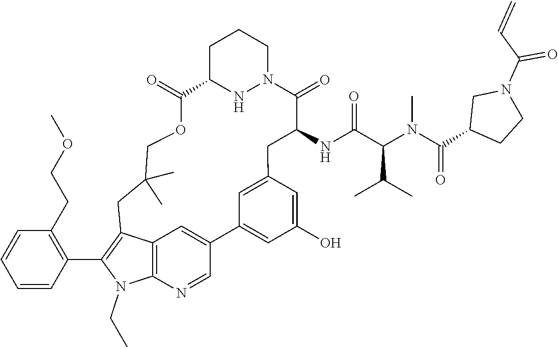 [SMILES: C=CC(=O)N1CC[C@H](C(=O)N(C)[C@H](C(=O)N[C@H]2Cc3cc(O)cc(c3)-c3cnc4c(c3)c(c(-c3ccccc3CCOC)n4CC)CC(C)(C)COC(=O)[C@@H]3CCCN(N3)C2=O)C(C)C)C1] |
| BA211 | 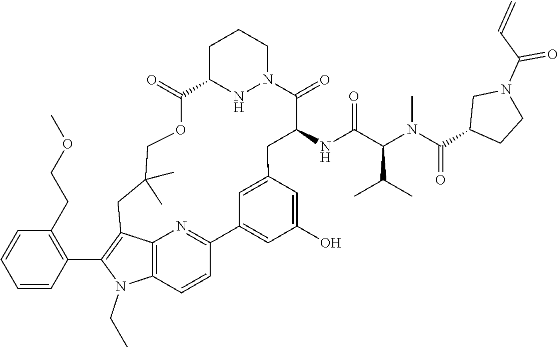 [SMILES: C=CC(=O)N1CC[C@H](C(=O)N(C)[C@H](C(=O)N[C@H]2Cc3cc(O)cc(c3)-c3ccc4c(n3)c(c(-c3ccccc3CCOC)n4CC)CC(C)(C)COC(=O)[C@@H]3CCCN(N3)C2=O)C(C)C)C1] |
| BA212 | 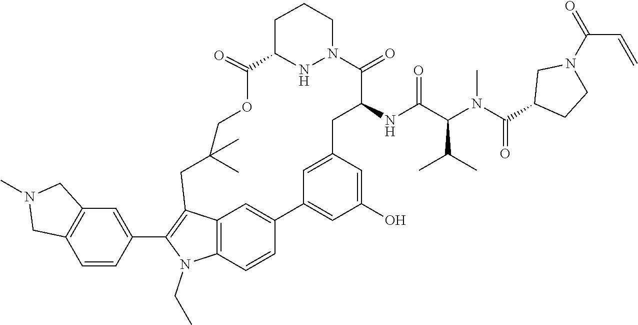 [SMILES: C=CC(=O)N1CC[C@H](C(=O)N(C)[C@H](C(=O)N[C@H]2Cc3cc(O)cc(c3)-c3ccc4c(c3)c(c(-c3ccc5c(c3)CN(C)C5)n4CC)CC(C)(C)COC(=O)[C@@H]3CCCN(N3)C2=O)C(C)C)C1] |
| BA213 | 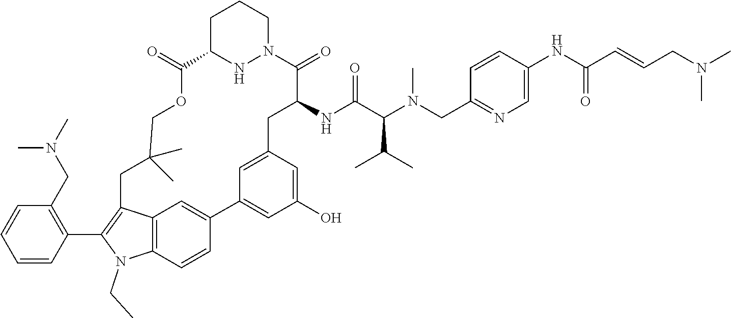 [SMILES: CCn1c(-c2ccccc2CN(C)C)c2c3cc(ccc31)-c1cc(O)cc(c1)C[C@H](NC(=O)[C@H](C(C)C)N(C)Cc1ccc(NC(=O)/C=C/CN(C)C)cn1)C(=O)N1CCC[C@H](N1)C(=O)OCC(C)(C)C2] |
| BA214 | 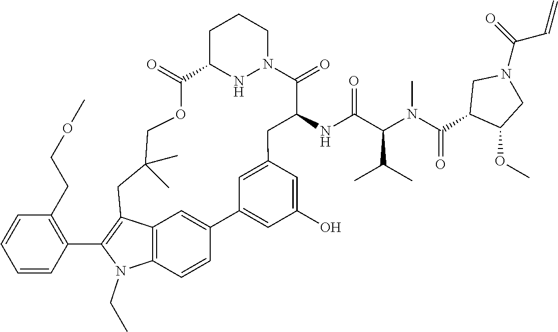 [SMILES: C=CC(=O)N1C[C@H](OC)[C@H](C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@@H]3CCCN(N3)C2=O)C(C)C)C1] |
| BA215 | 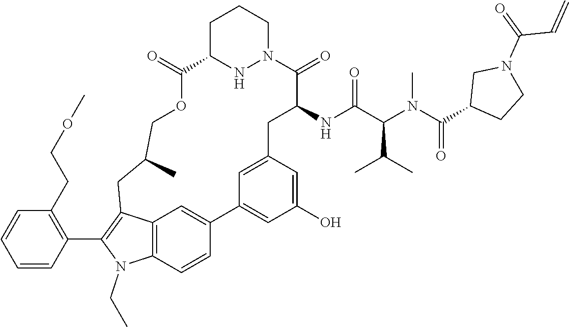 [SMILES: C=CC(=O)N1CC[C@H](C(=O)N(C)[C@H](C(=O)N[C@H]2Cc3cc(O)cc(c3)-c3ccc4c(c3)c(c(-c3ccccc3CCOC)n4CC)C[C@H](C)COC(=O)[C@@H]3CCCN(N3)C2=O)C(C)C)C1] |
| BA216 | 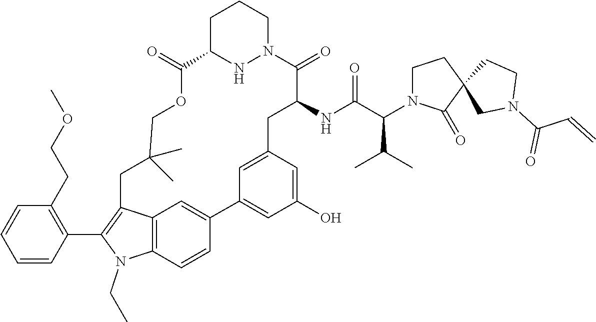 [SMILES: C=CC(=O)N1CC[C@]2(CCN([C@H](C(=O)N[C@H]3Cc4cc(O)cc(c4)-c4ccc5c(c4)c(c(-c4ccccc4CCOC)n5CC)CC(C)(C)COC(=O)[C@@H]4CCCN(N4)C3=O)C(C)C)C2=O)C1] |
| BA217 | 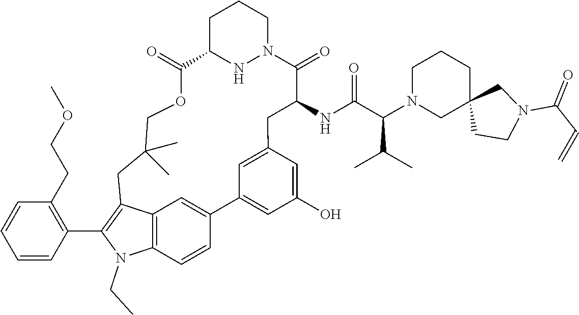 [SMILES: C=CC(=O)N1CC[C@]2(CCCN([C@H](C(=O)N[C@H]3Cc4cc(O)cc(c4)-c4ccc5c(c4)c(c(-c4ccccc4CCOC)n5CC)CC(C)(C)COC(=O)[C@@H]4CCCN(N4)C3=O)C(C)C)C2)C1] |
| BA218 | 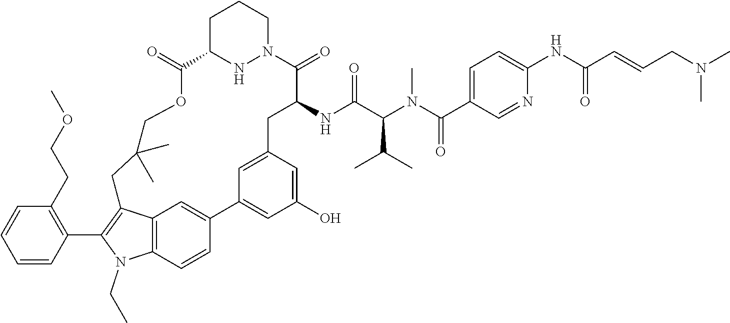 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(NC(=O)/C=C/CN(C)C)nc1)C(=O)N1CCC[C@H](N1)C(=O)OCC(C)(C)C2] |
| BA219 | 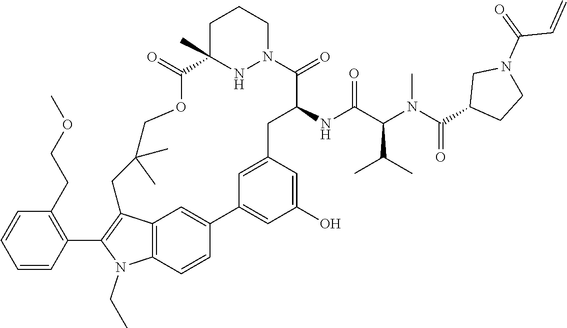 [SMILES: C=CC(=O)N1CC[C@H](C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@]3(C)CCCN(N3)C2=O)C(C)C)C1] |
| BA220 | 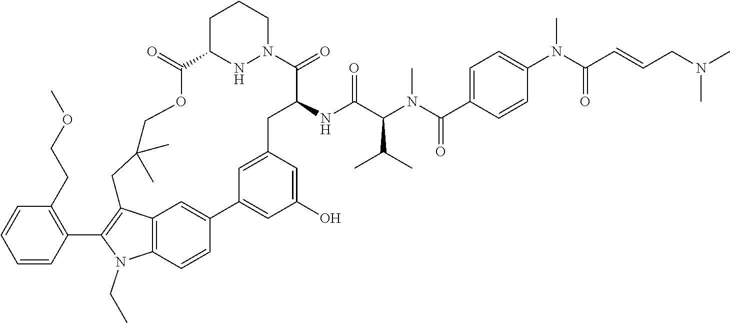 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(N(C)C(=O)/C=C/CN(C)C)cc1)C(=O)N1CCC[C@H](N1)C(=O)OCC(C)(C)C2] |
| BA221 | 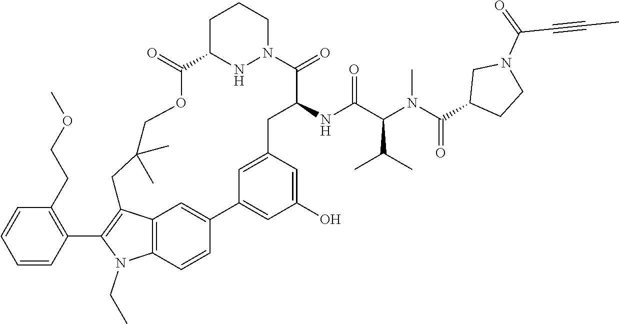 [SMILES: CC#CC(=O)N1CC[C@H](C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@@H]3CCCN(N3)C2=O)C(C)C)C1] |
| BA222 | 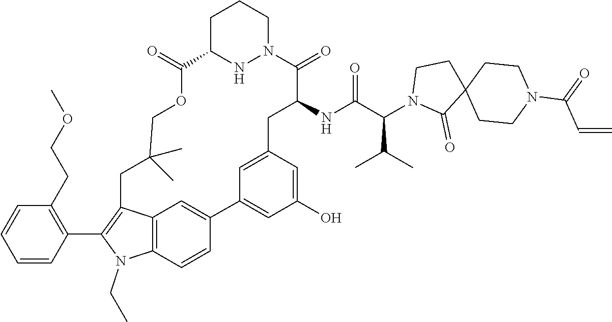 [SMILES: C=CC(=O)N1CCC2(CC1)CCN([C@H](C(=O)N[C@H]1Cc3cc(O)cc(c3)-c3ccc4c(c3)c(c(-c3ccccc3CCOC)n4CC)CC(C)(C)COC(=O)[C@@H]3CCCN(N3)C1=O)C(C)C)C2=O] |
| BA223 | 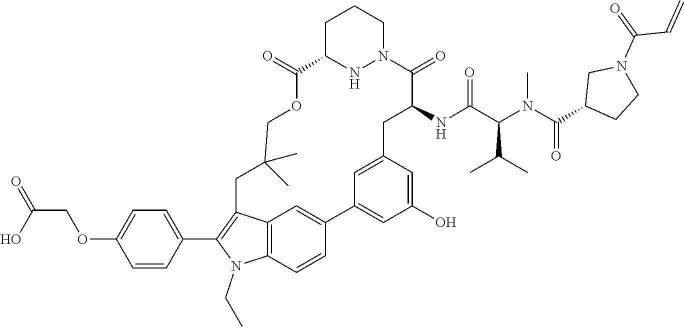 [SMILES: C=CC(=O)N1CC[C@H](C(=O)N(C)[C@H](C(=O)N[C@H]2Cc3cc(O)cc(c3)-c3ccc4c(c3)c(c(-c3ccc(OCC(=O)O)cc3)n4CC)CC(C)(C)COC(=O)[C@@H]3CCCN(N3)C2=O)C(C)C)C1] |
| BA224 | 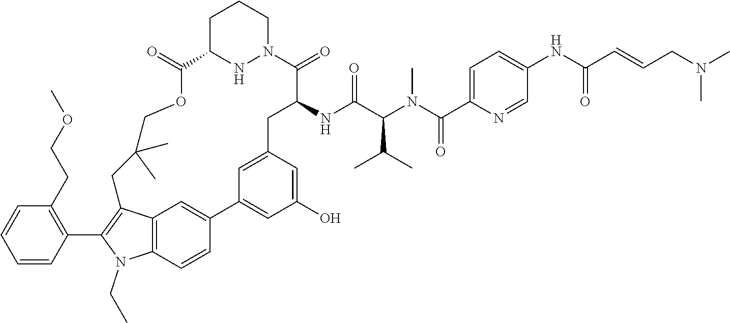 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(NC(=O)/C=C/CN(C)C)cn1)C(=O)N1CCC[C@H](N1)C(=O)OCC(C)(C)C2] |
| BA225 | 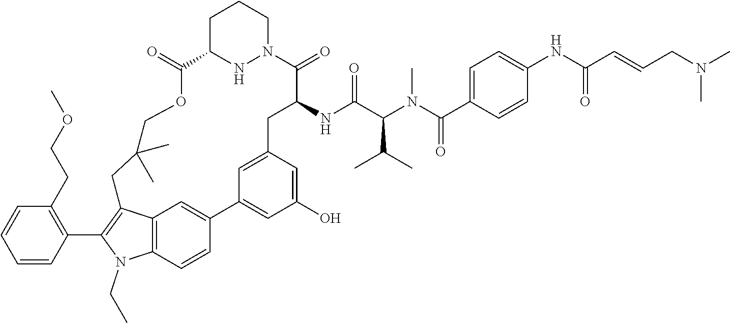 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(NC(=O)/C=C/CN(C)C)cc1)C(=O)N1CCC[C@H](N1)C(=O)OCC(C)(C)C2] |
| BA226 | 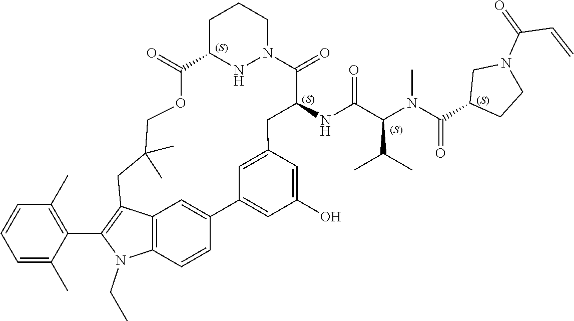 |
| BA227 | 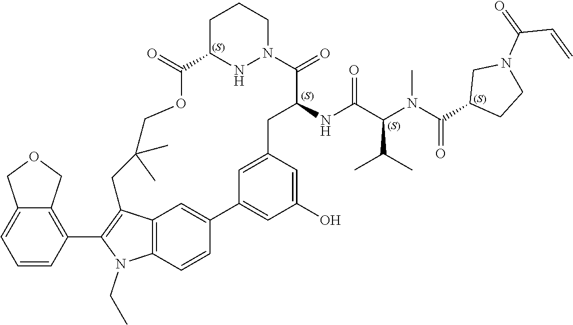 |
| BA228 | 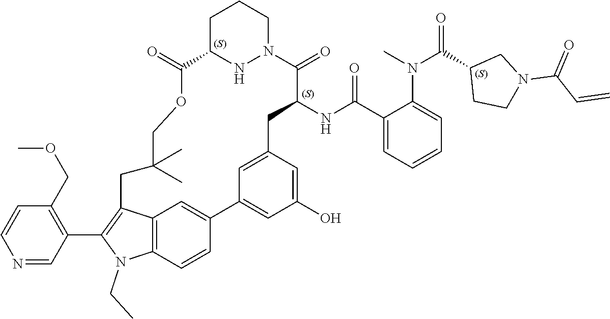 |
| BA229 | 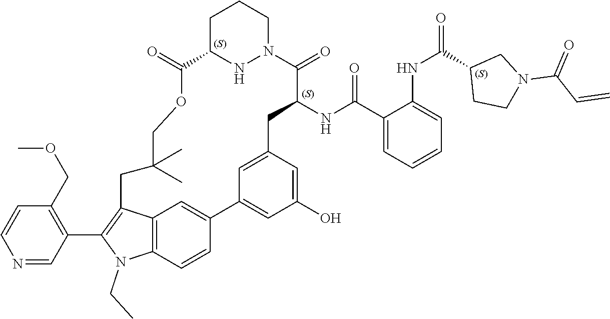 |
| BA230 | 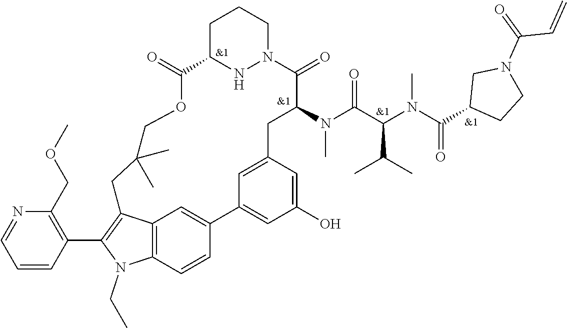 |
| BA231 | 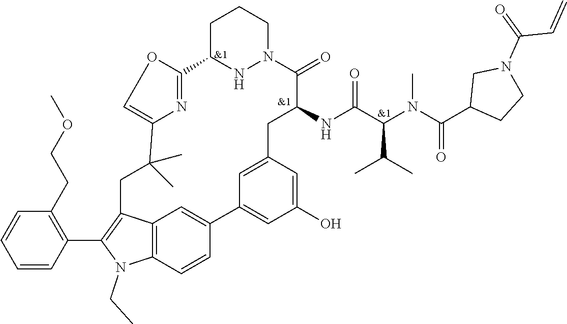 [SMILES: C=CC(=O)N1CCC(C(=O)N(C)[C@H](C(=O)N[C@H]2Cc3cc(O)cc(c3)-c3ccc4c(c3)c(c(-c3ccccc3CCOC)n4CC)CC(C)(C)c3coc(n3)[C@@H]3CCCN(N3)C2=O)C(C)C)C1] |
| BA232 | 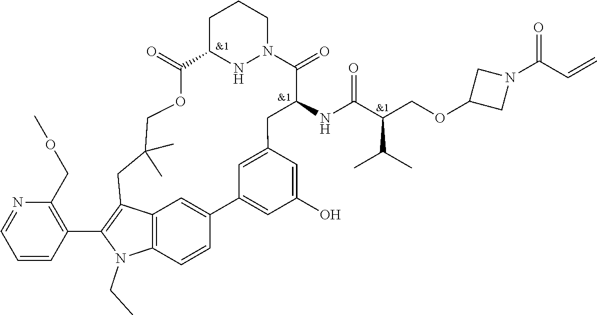 [SMILES: C=CC(=O)N1CC(OC[C@H](C(=O)N[C@H]2Cc3cc(O)cc(c3)-c3ccc4c(c3)c(c(-c3cccnc3COC)n4CC)CC(C)(C)COC(=O)[C@@H]3CCCN(N3)C2=O)C(C)C)C1] |
| BA233 | 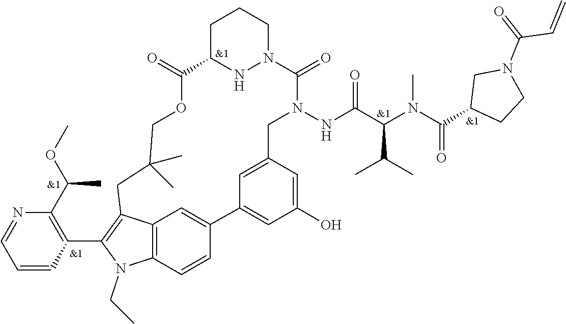 |
| BA234 | 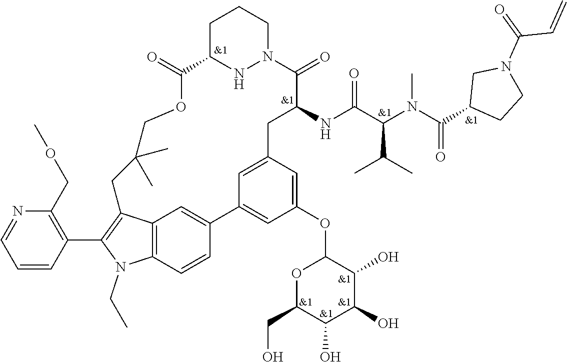 [SMILES: C=CC(=O)N1CC[C@H](C(=O)N(C)[C@H](C(=O)N[C@H]2Cc3cc(OC4O[C@H](CO)[C@@H](O)[C@H](O)[C@H]4O)cc(c3)-c3ccc4c(c3)c(c(-c3cccnc3COC)n4CC)CC(C)(C)COC(=O)[C@@H]3CCCN(N3)C2=O)C(C)C)C1] |
| BA235 | 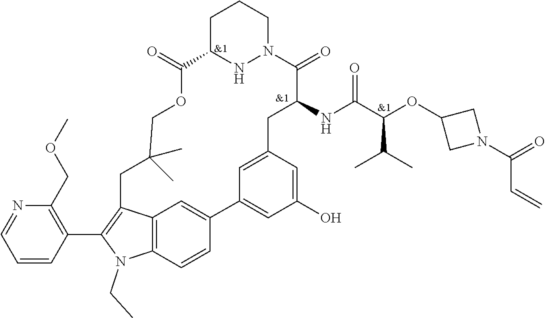 [SMILES: C=CC(=O)N1CC(O[C@H](C(=O)N[C@H]2Cc3cc(O)cc(c3)-c3ccc4c(c3)c(c(-c3cccnc3COC)n4CC)CC(C)(C)COC(=O)[C@@H]3CCCN(N3)C2=O)C(C)C)C1] |
| BA236 | 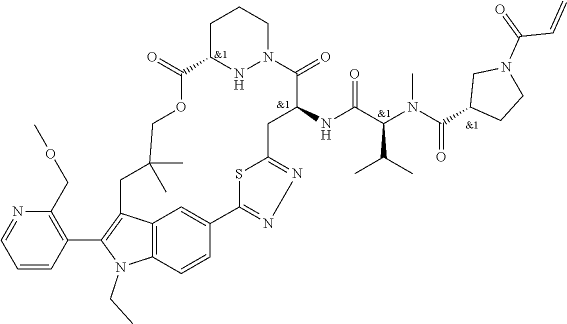 [SMILES: C=CC(=O)N1CC[C@H](C(=O)N(C)[C@H](C(=O)N[C@H]2CC3=NN=C(S3)c3ccc4c(c3)c(c(-c3cccnc3COC)n4CC)CC(C)(C)COC(=O)[C@@H]3CCCN(N3)C2=O)C(C)C)C1] |
| BA237 | 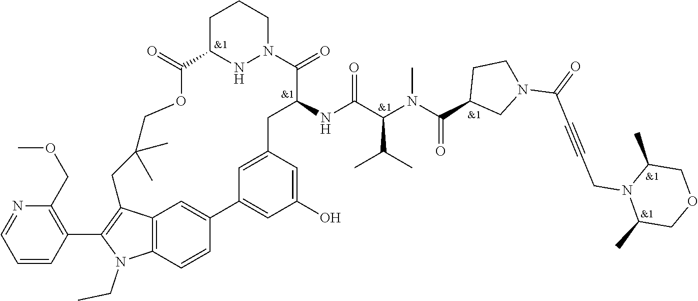 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CCN3[C@H](C)COC[C@@H]3C)C1)C(=O)N1CCC[C@H](N1)C(=O)OCC(C)(C)C2] |
| BA238 | 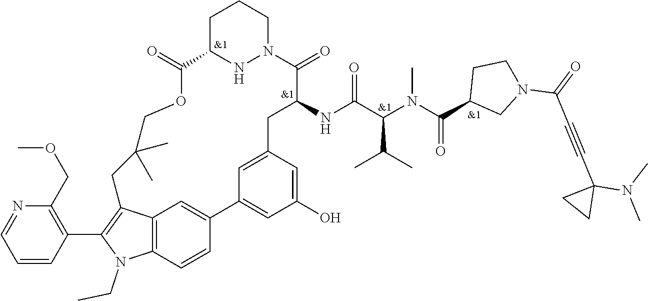 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CC3(N(C)C)CC3)C1)C(=O)N1CCC[C@H](N1)C(=O)OCC(C)(C)C2] |
| BA239 | 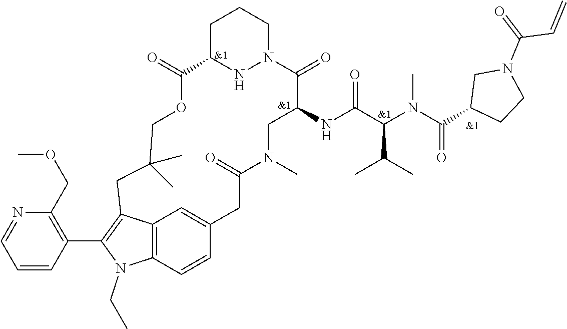 [SMILES: C=CC(=O)N1CC[C@H](C(=O)N(C)[C@H](C(=O)N[C@H]2CN(C)C(=O)Cc3ccc4c(c3)c(c(-c3cccnc3COC)n4CC)CC(C)(C)COC(=O)[C@@H]3CCCN(N3)C2=O)C(C)C)C1] |
| BA240 | 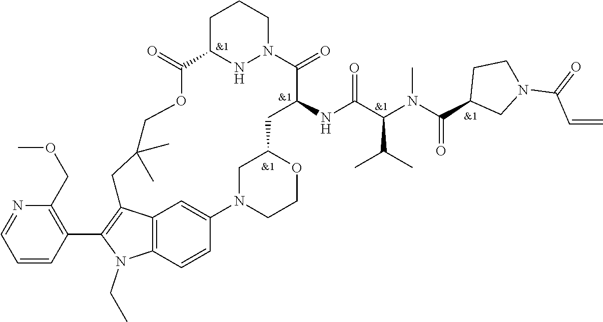 |
| BA241 | 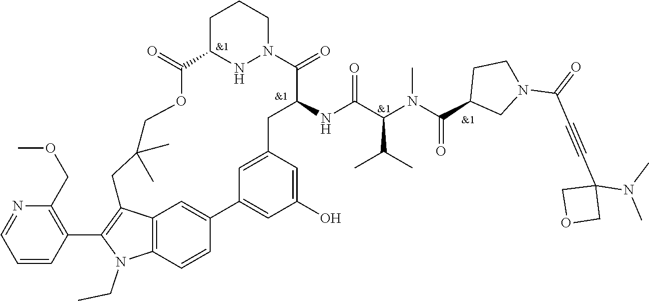 |
| BA242 | 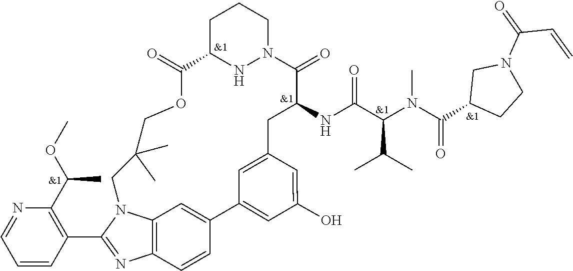 [SMILES: C=CC(=O)N1CC[C@H](C(=O)N(C)[C@H](C(=O)N[C@H]2Cc3cc(O)cc(c3)-c3ccc4nc(-c5cccnc5[C@H](C)OC)n(c4c3)CC(C)(C)COC(=O)[C@@H]3CCCN(N3)C2=O)C(C)C)C1] |
| BA243 | 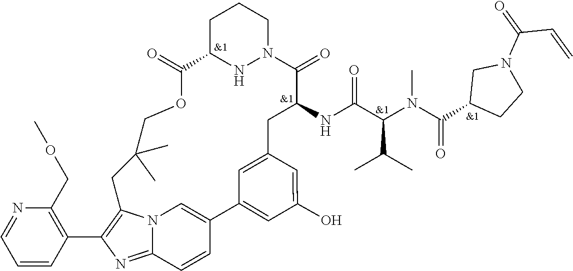 [SMILES: C=CC(=O)N1CC[C@H](C(=O)N(C)[C@H](C(=O)N[C@H]2Cc3cc(O)cc(c3)-c3ccc4nc(-c5cccnc5COC)c(n4c3)CC(C)(C)COC(=O)[C@@H]3CCCN(N3)C2=O)C(C)C)C1] |
| BA244 | 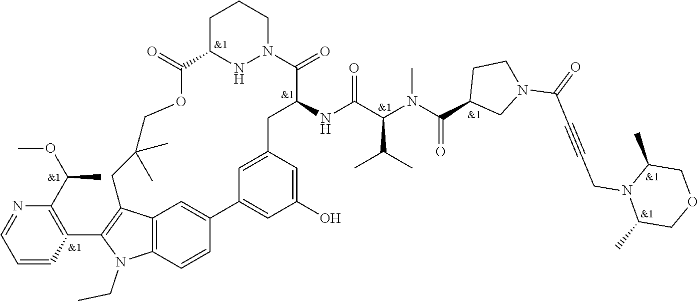 |
| BA245 | 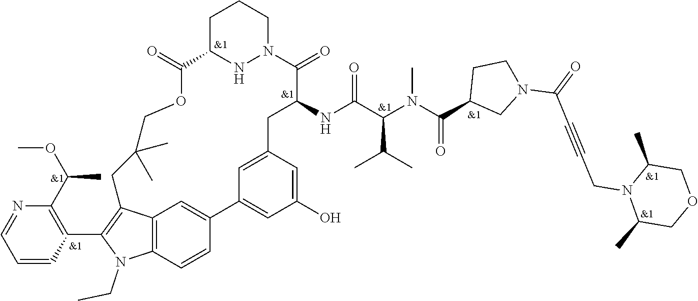 |
| BA246 | 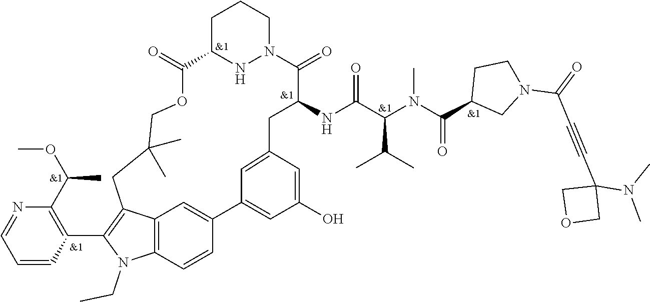 |
| BA247 | 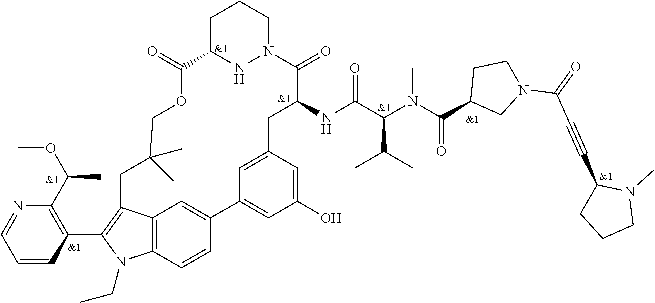 |
| BA248 | 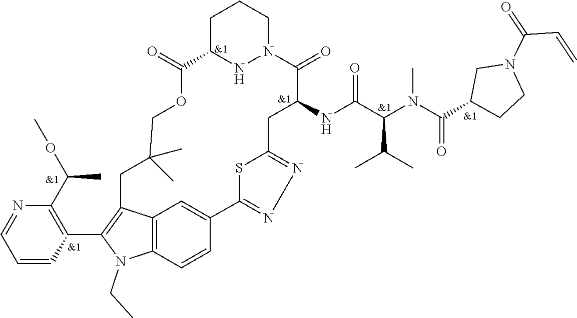 |
| BA249 | 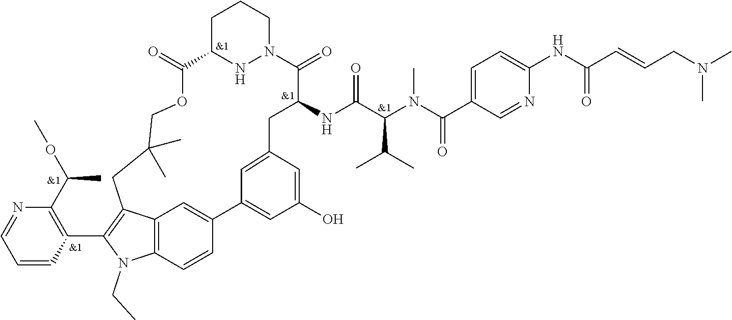 |
| BA250 | 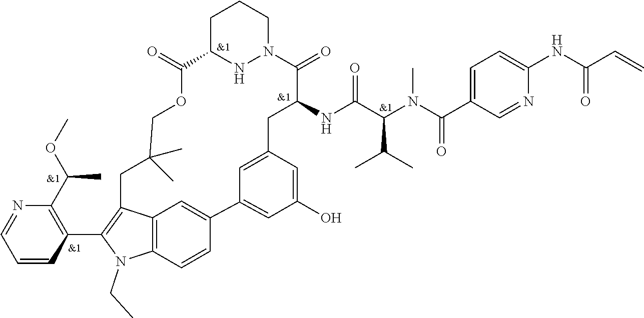 |
| BA251 | 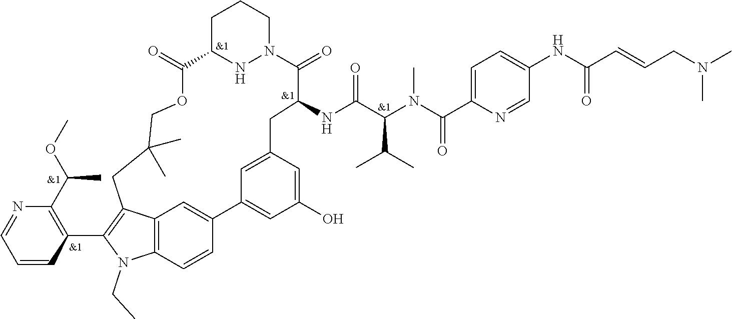 |
| BA252 | 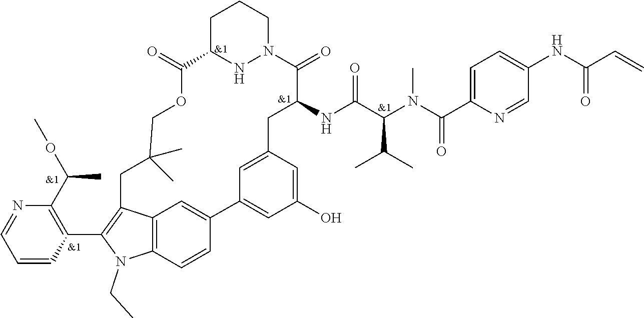 |
| BA253 | 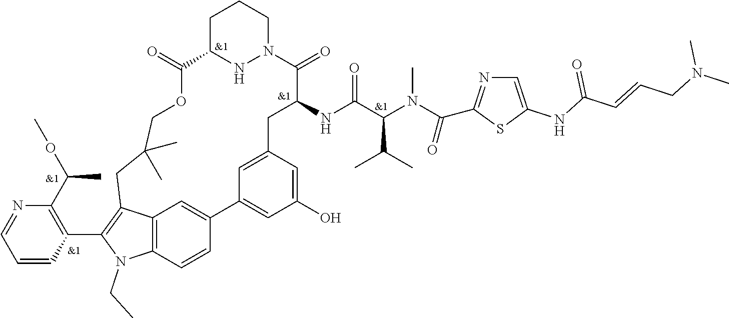 |
| BA254 | 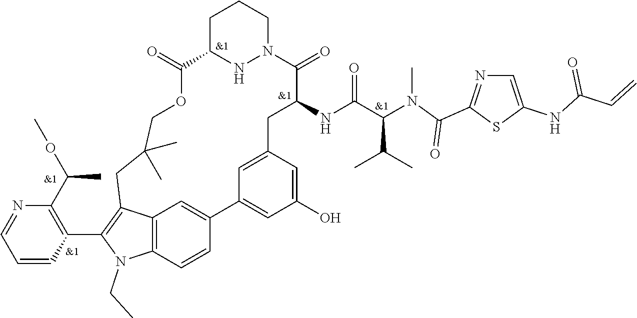 |
| BA255 | 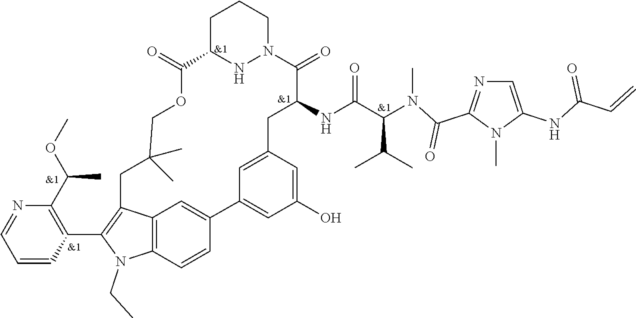 |
| BA256 | 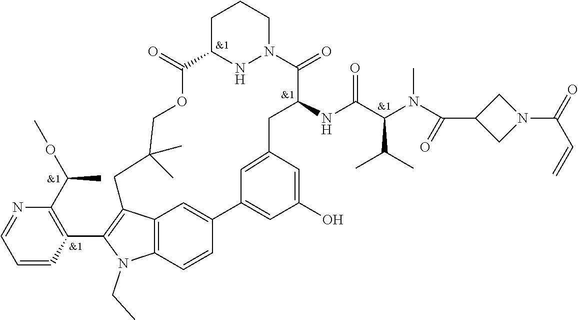 |
| BA257 | 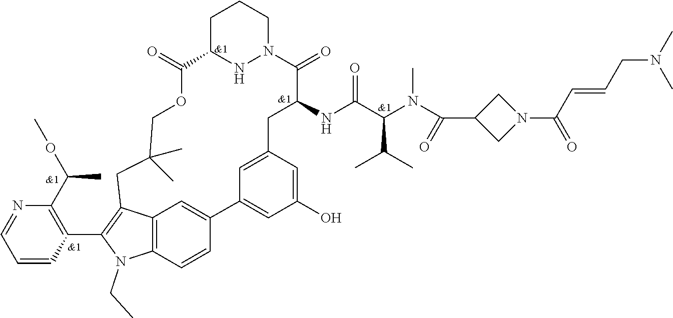 |
| BA258 | 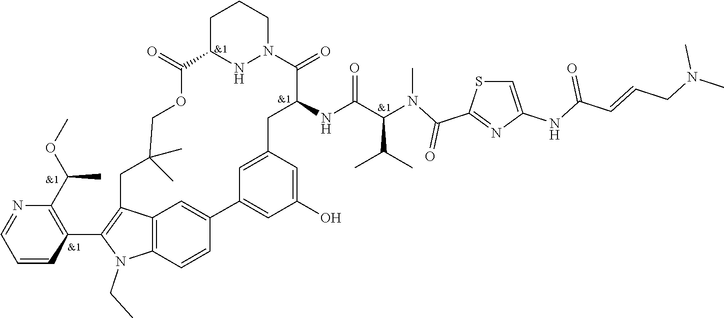 |
| BA259 | 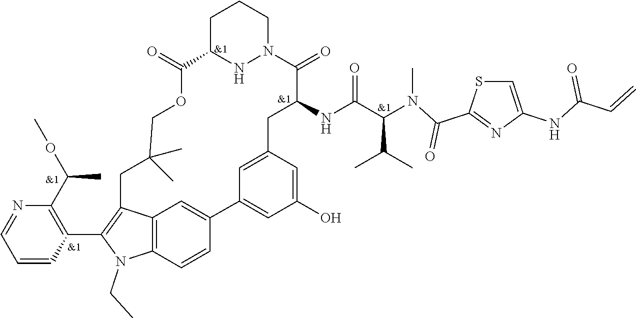 |
| BA260 | 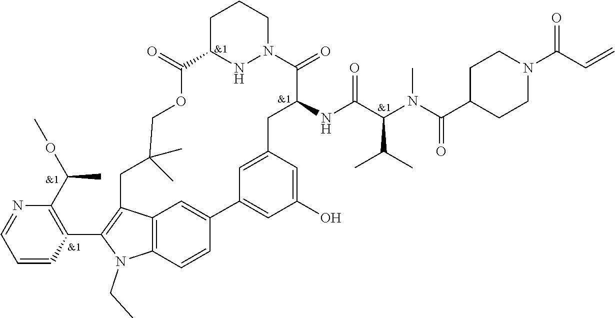 |
| BA261 | 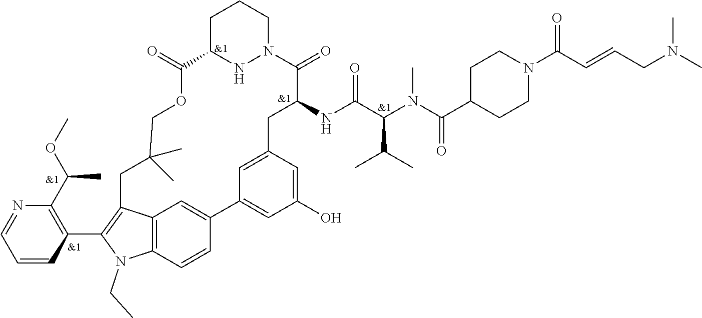 |
| BA262 | 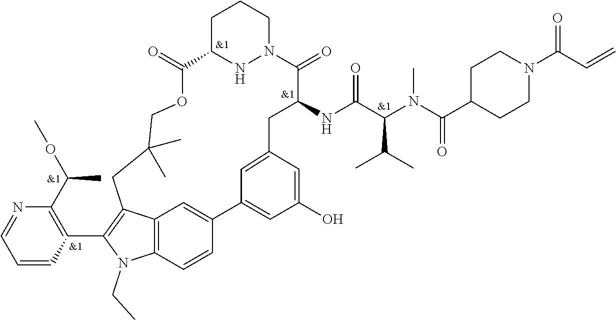 |
| BA263 | 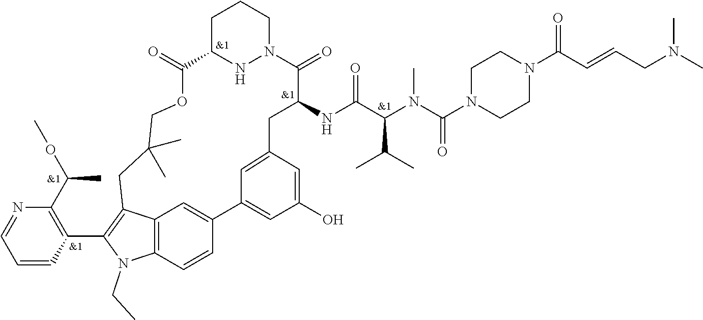 |
| BA264 | 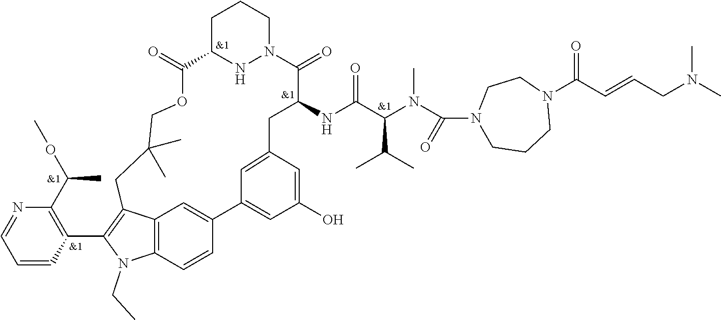 |
| BA265 | 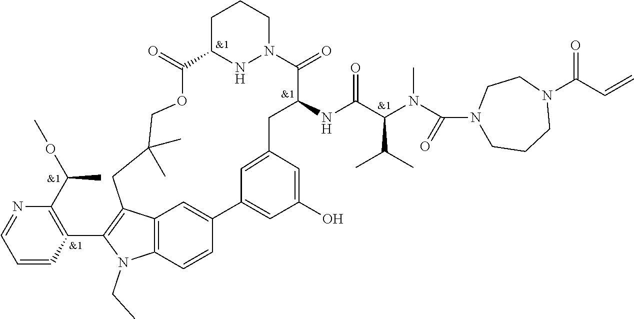 |
| BA266 | 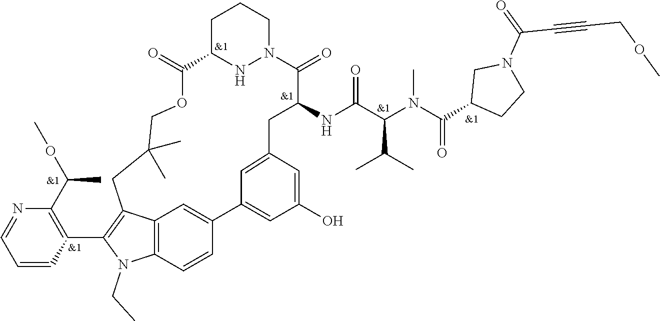 |
| BA267 | 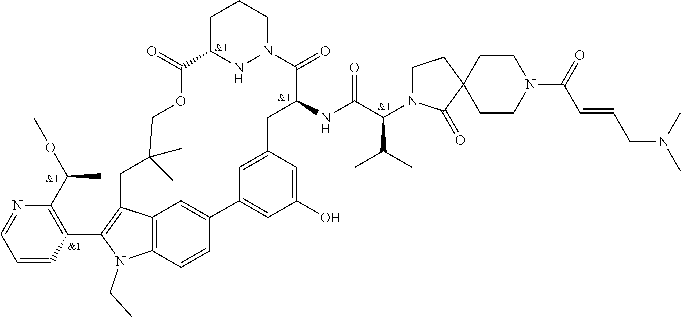 |
| BA268 | 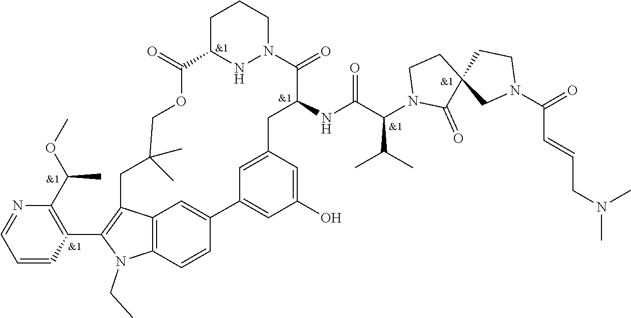 |
| BA269 | 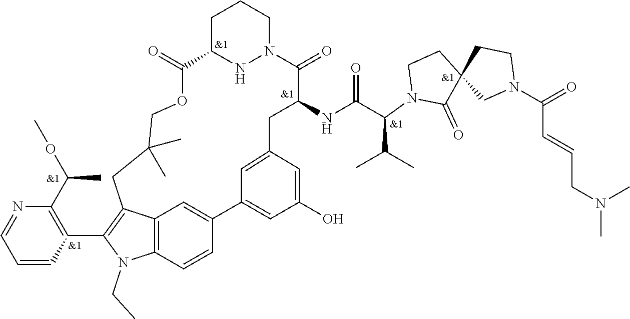 |
| BA270 | 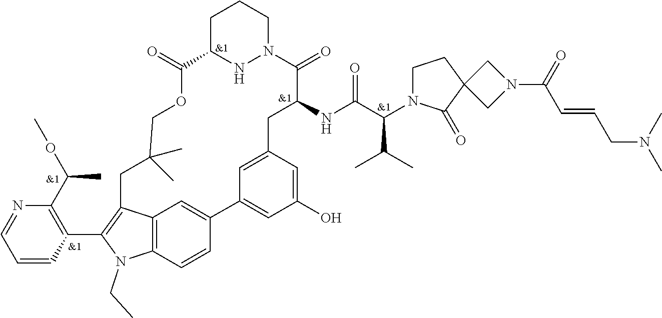 |
| BA271 | 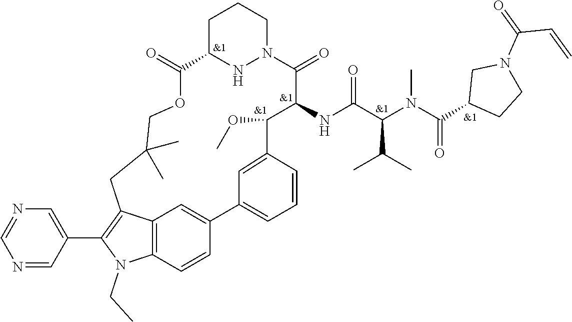 [SMILES: C=CC(=O)N1CC[C@H](C(=O)N(C)[C@H](C(=O)N[C@@H]2C(=O)N3CCC[C@H](N3)C(=O)OCC(C)(C)Cc3c(-c4cncnc4)n(CC)c4ccc(cc34)-c3cccc(c3)[C@@H]2OC)C(C)C)C1] |
| BA272 | 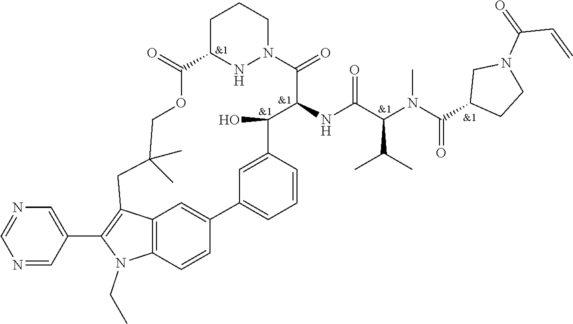 [SMILES: C=CC(=O)N1CC[C@H](C(=O)N(C)[C@H](C(=O)N[C@@H]2C(=O)N3CCC[C@H](N3)C(=O)OCC(C)(C)Cc3c(-c4cncnc4)n(CC)c4ccc(cc34)-c3cccc(c3)[C@H]2O)C(C)C)C1] |
| BA273 | 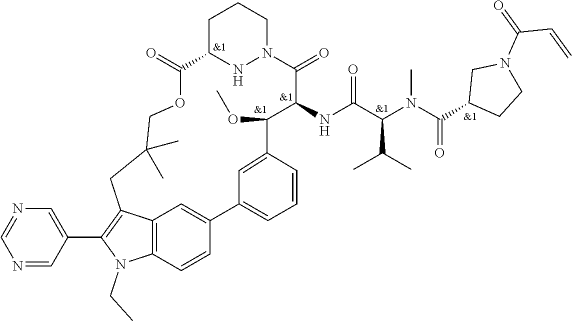 [SMILES: C=CC(=O)N1CC[C@H](C(=O)N(C)[C@H](C(=O)N[C@@H]2C(=O)N3CCC[C@H](N3)C(=O)OCC(C)(C)Cc3c(-c4cncnc4)n(CC)c4ccc(cc34)-c3cccc(c3)[C@H]2OC)C(C)C)C1] |
| BA274 | 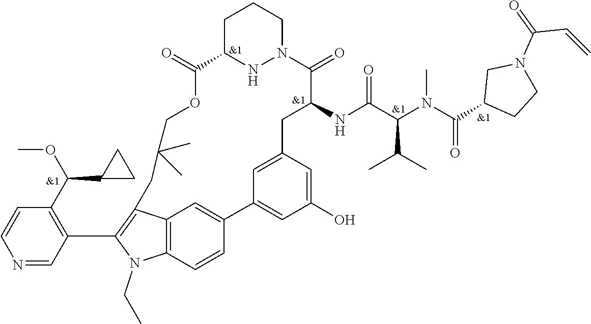 [SMILES: C=CC(=O)N1CC[C@H](C(=O)N(C)[C@H](C(=O)N[C@H]2Cc3cc(O)cc(c3)-c3ccc4c(c3)c(c(-c3cnccc3[C@@H](OC)C3CC3)n4CC)CC(C)(C)COC(=O)[C@@H]3CCCN(N3)C2=O)C(C)C)C1] |
| BA275 | 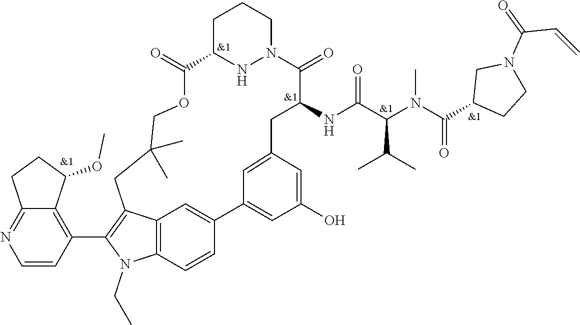 [SMILES: C=CC(=O)N1CC[C@H](C(=O)N(C)[C@H](C(=O)N[C@H]2Cc3cc(O)cc(c3)-c3ccc4c(c3)c(c(-c3ccnc5c3[C@@H](OC)CC5)n4CC)CC(C)(C)COC(=O)[C@@H]3CCCN(N3)C2=O)C(C)C)C1] |
| BA276 | 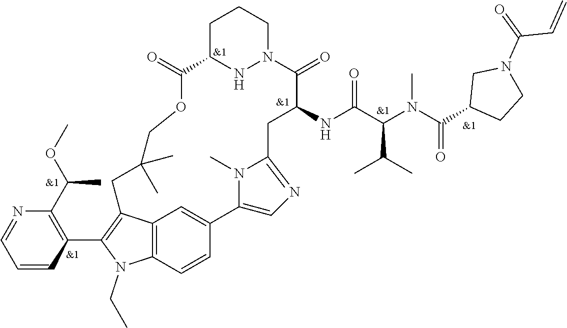 |
| BA277 | 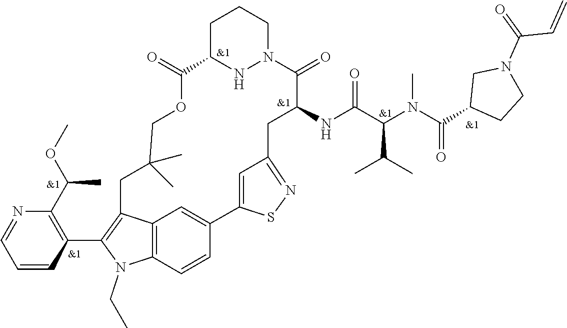 |
| BA278 | 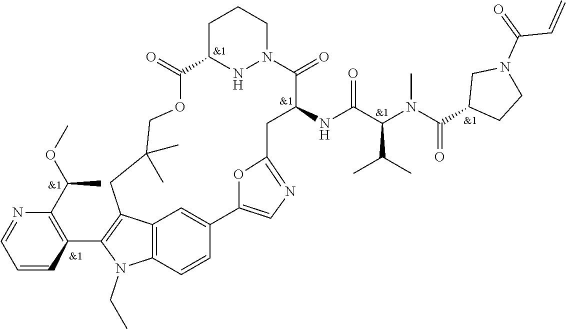 |
| BA279 | 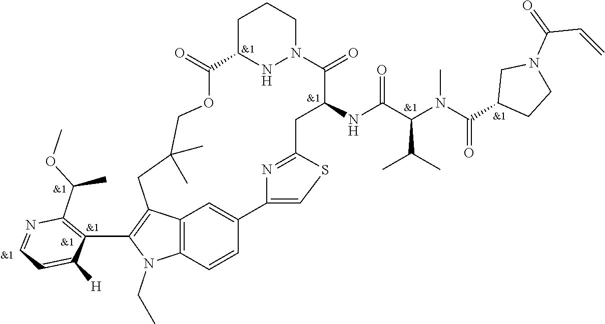 |
| BA280 | 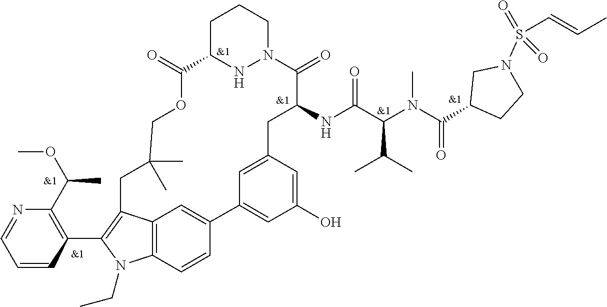 |
| BA281 | 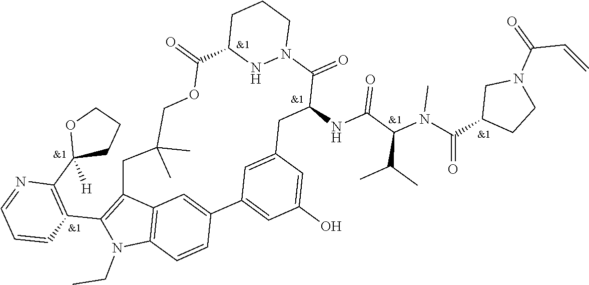 |
| BA282 | 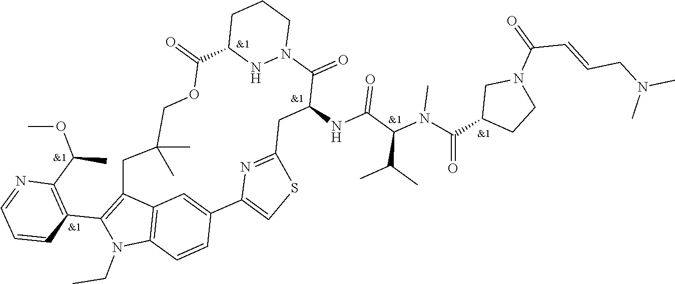 |
| BA283 | 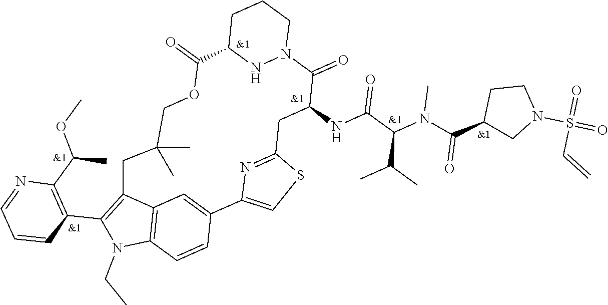 |
| BA284 | 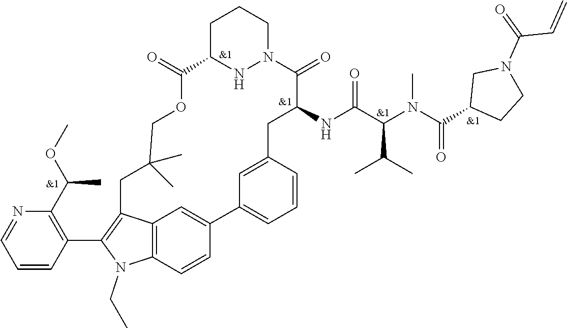 [SMILES: C=CC(=O)N1CC[C@H](C(=O)N(C)[C@H](C(=O)N[C@H]2Cc3cccc(c3)-c3ccc4c(c3)c(c(-c3cccnc3[C@H](C)OC)n4CC)CC(C)(C)COC(=O)[C@@H]3CCCN(N3)C2=O)C(C)C)C1] |
| BA285 | 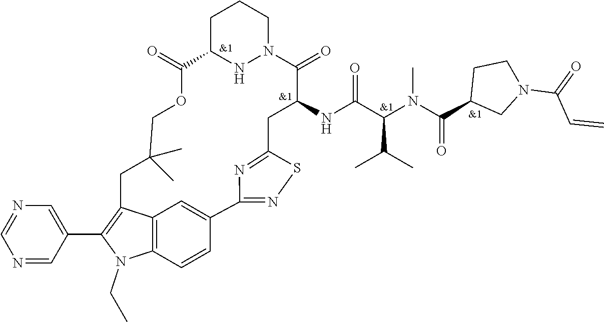 [SMILES: C=CC(=O)N1CC[C@H](C(=O)N(C)[C@H](C(=O)N[C@H]2Cc3nc(ns3)-c3ccc4c(c3)c(c(-c3cncnc3)n4CC)CC(C)(C)COC(=O)[C@@H]3CCCN(N3)C2=O)C(C)C)C1] |
| BA286 | 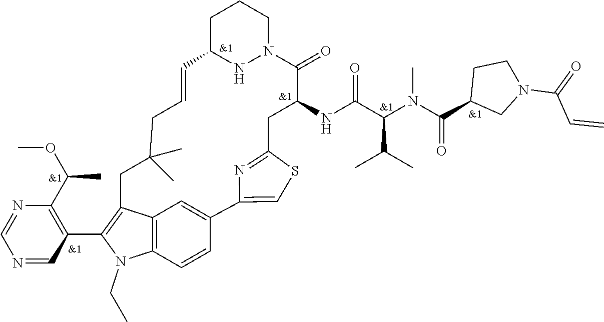 |
| BA287 | 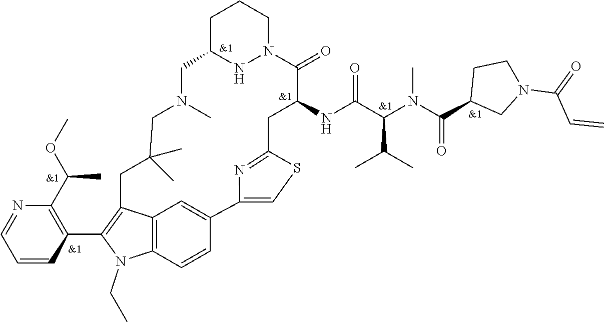 |
| BA288 | 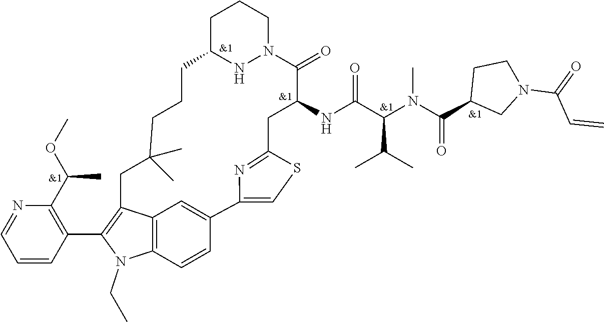 [SMILES: C=CC(=O)N1CC[C@H](C(=O)N(C)[C@H](C(=O)N[C@H]2Cc3nc(cs3)-c3ccc4c(c3)c(c(-c3cccnc3[C@H](C)OC)n4CC)CC(C)(C)CCC[C@@H]3CCCN(N3)C2=O)C(C)C)C1] |
| BA289 | 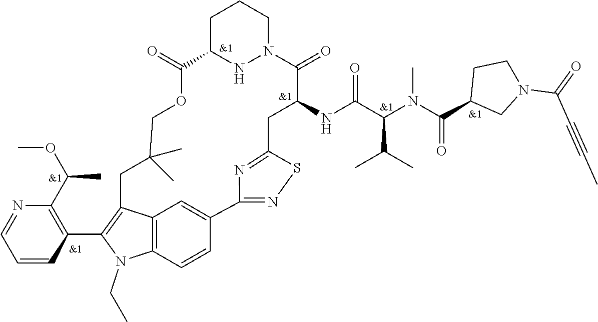 |
| BA290 | 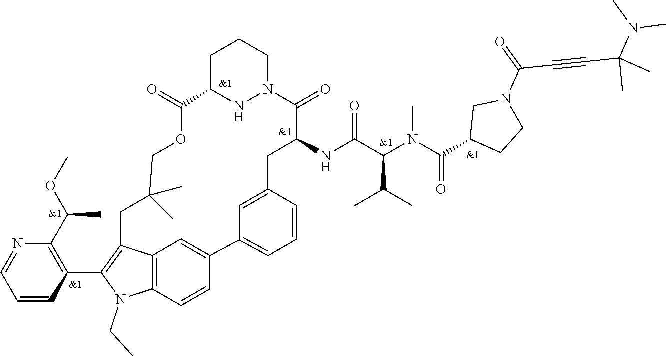 |
| BA291 | 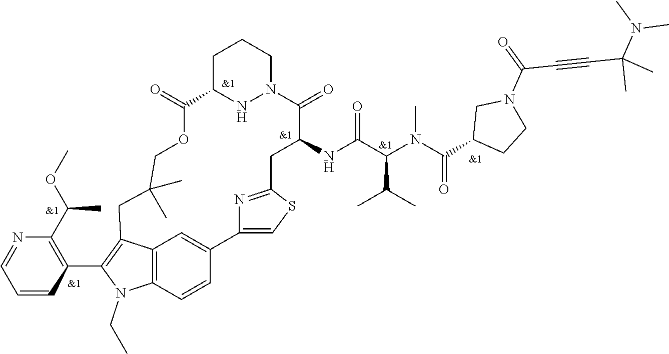 |
| BA292 | 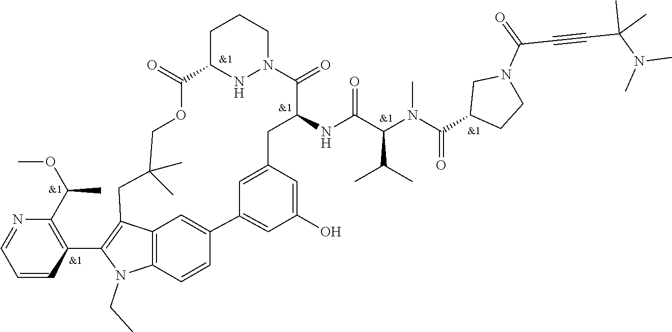 |
| BA293 | 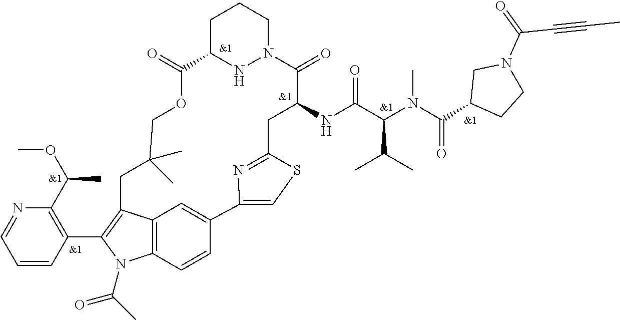 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4C(C)=O)CC(C)(C)COC(=O)[C@@H]3CCCN(N3)C2=O)C(C)C)C1] |
| BA294 | 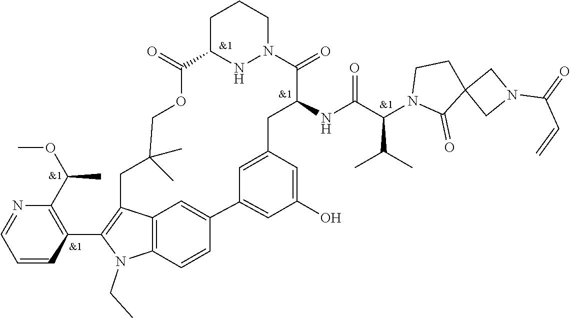 |
| BA295 | 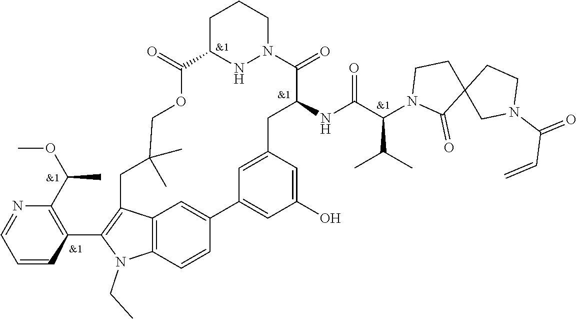 |
| BA296 | 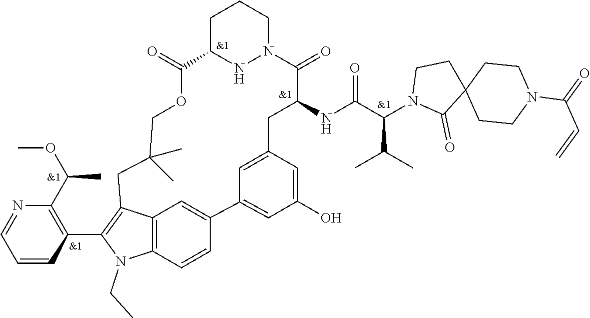 |
| BA297 | 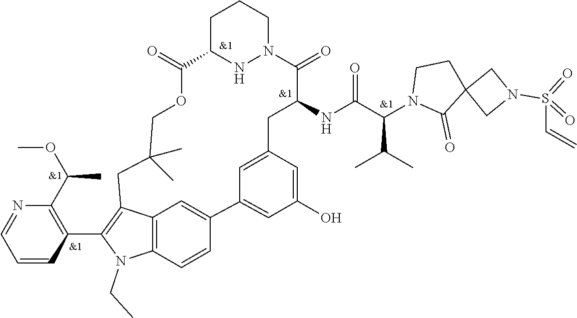 |
| BA298 | 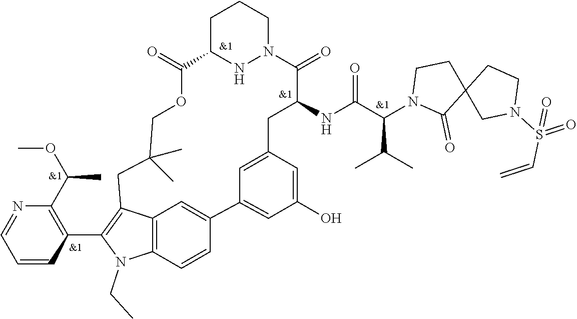 |
| BA299 | 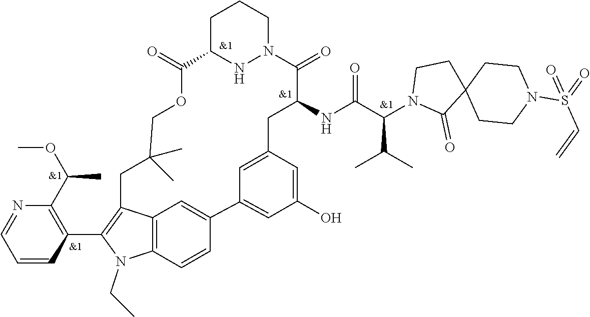 |
| BA300 | 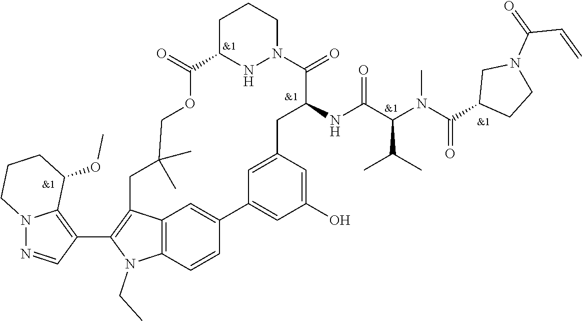 |
| BA301 | 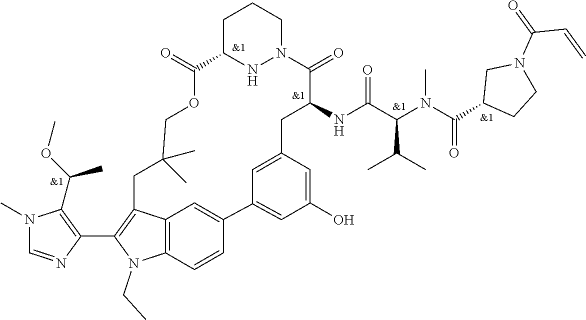 [SMILES: C=CC(=O)N1CC[C@H](C(=O)N(C)[C@H](C(=O)N[C@H]2Cc3cc(O)cc(c3)-c3ccc4c(c3)c(c(-c3ncn(C)c3[C@H](C)OC)n4CC)CC(C)(C)COC(=O)[C@@H]3CCCN(N3)C2=O)C(C)C)C1] |
| BA302 | 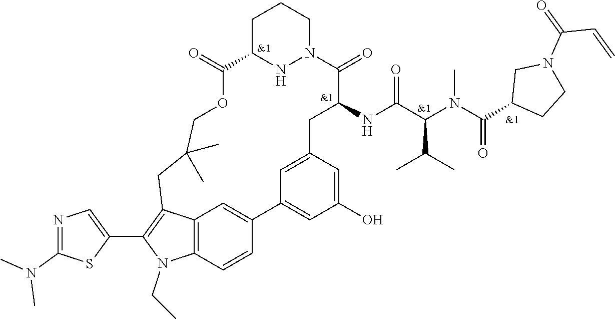 |
| BA303 | 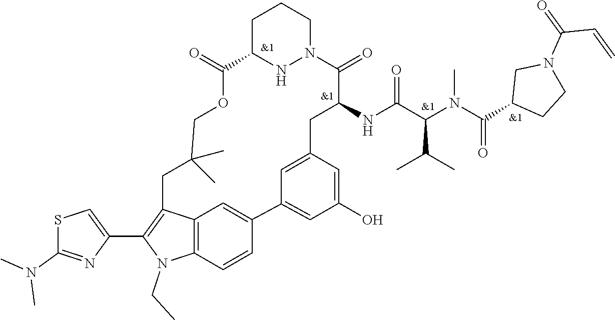 [SMILES: C=CC(=O)N1CC[C@H](C(=O)N(C)[C@H](C(=O)N[C@H]2Cc3cc(O)cc(c3)-c3ccc4c(c3)c(c(-c3csc(N(C)C)n3)n4CC)CC(C)(C)COC(=O)[C@@H]3CCCN(N3)C2=O)C(C)C)C1] |
| BA304 | 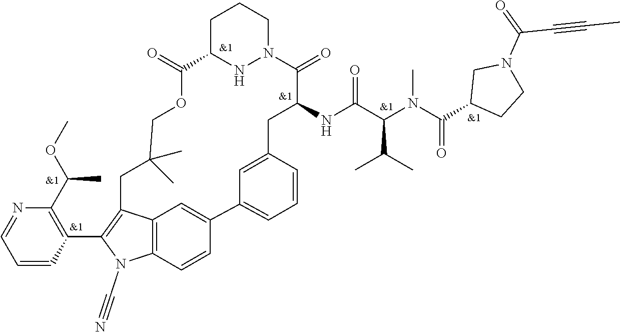 |
| BA305 | 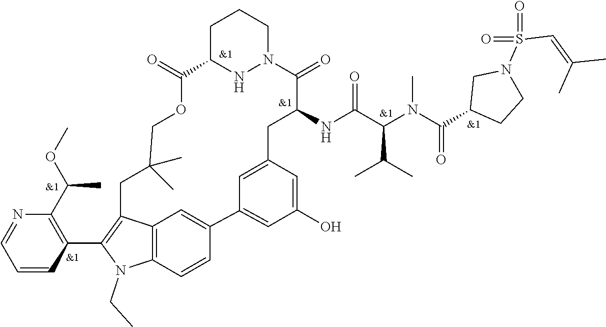 |
| BA306 | 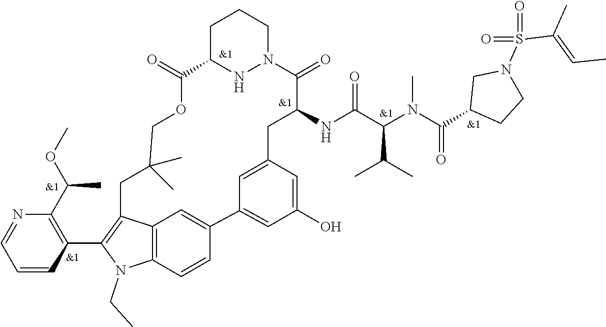 |
| BA307 | 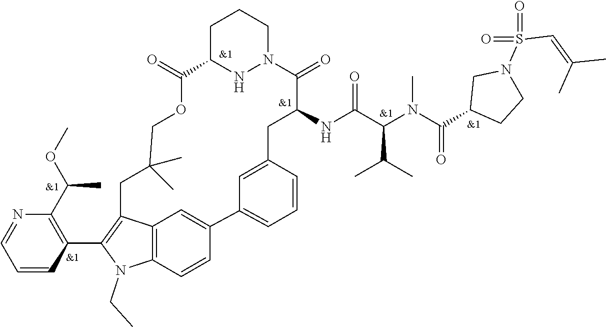 |
| BA308 | 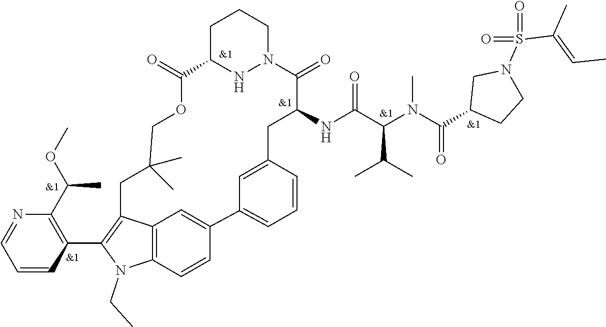 |
| BA309 | 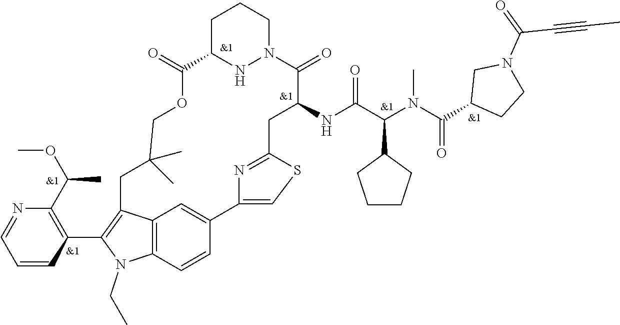 |
| BA310 | 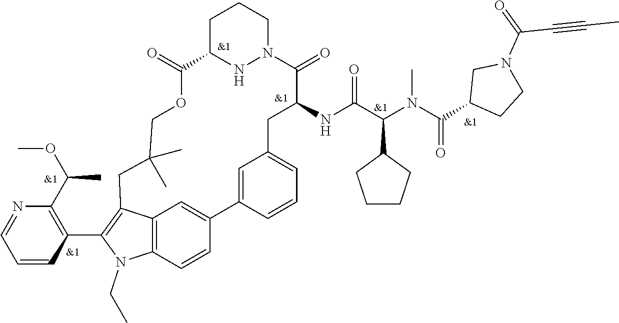 |
| BA311 | 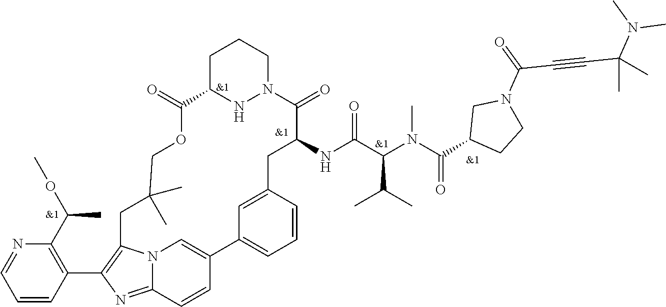 |
| BA312 | 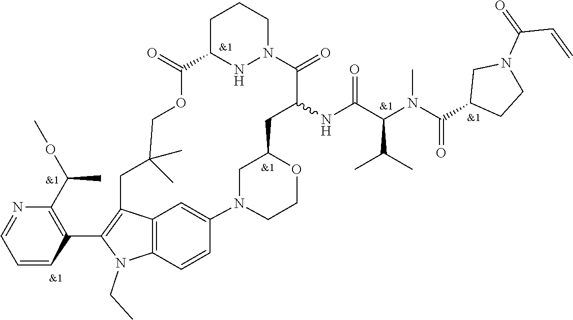 |
| BA313 | 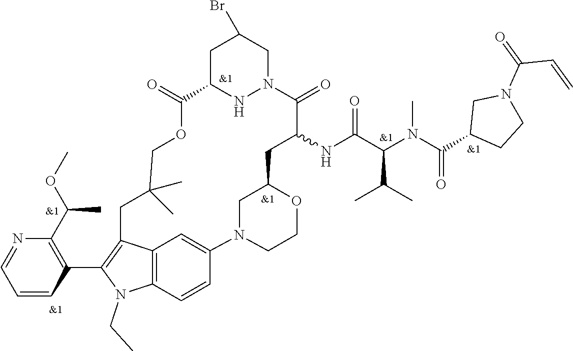 |
| BA314 | 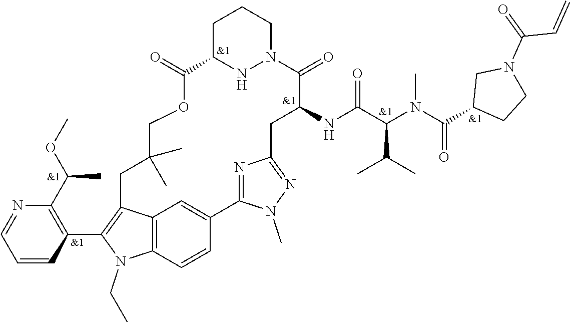 |
| BA316 | 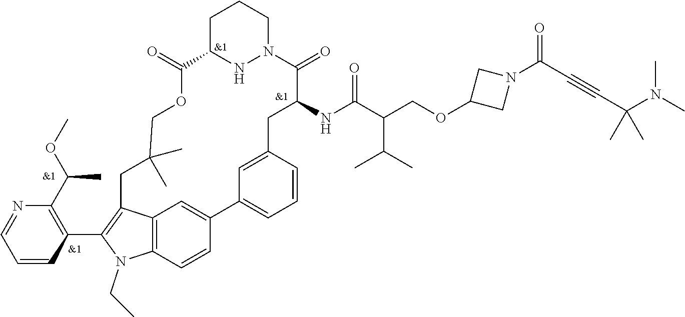 |
| BA317 | 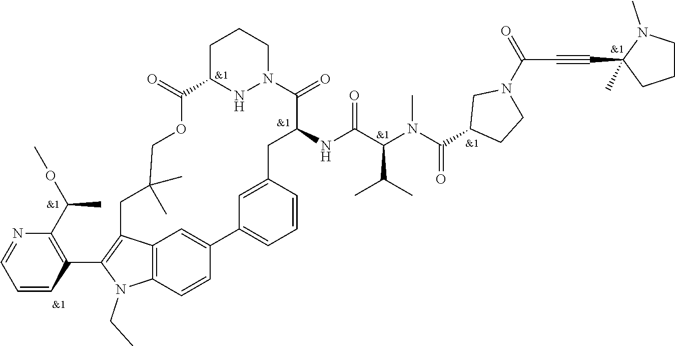 |
| BA318 | 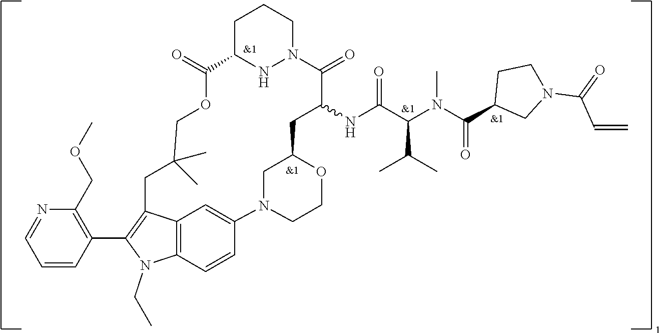 |
| BA319 | 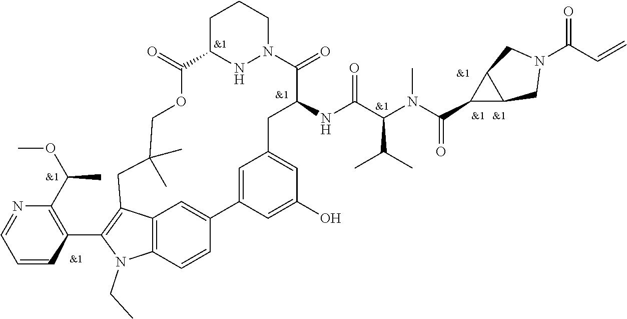 |
| BA320 | 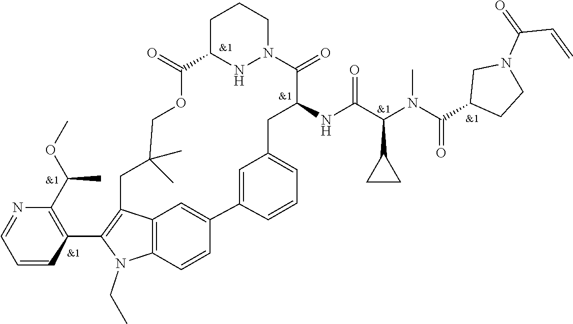 |
| BA321 | 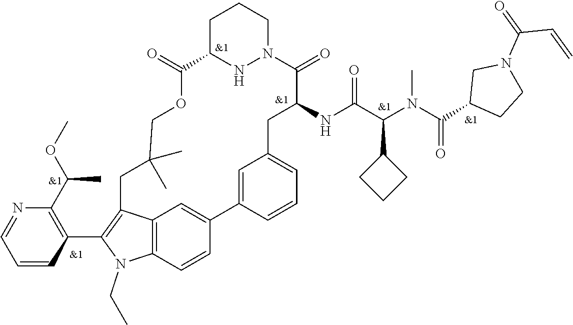 |
| BA322 | 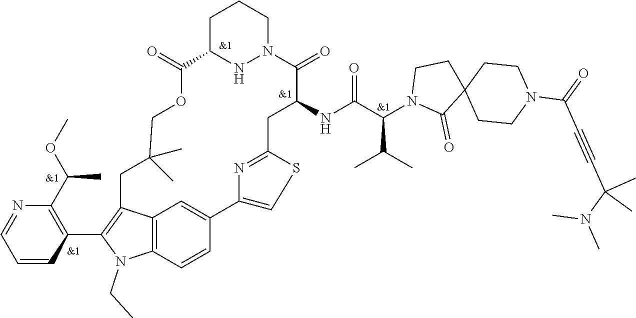 |
| BA323 | 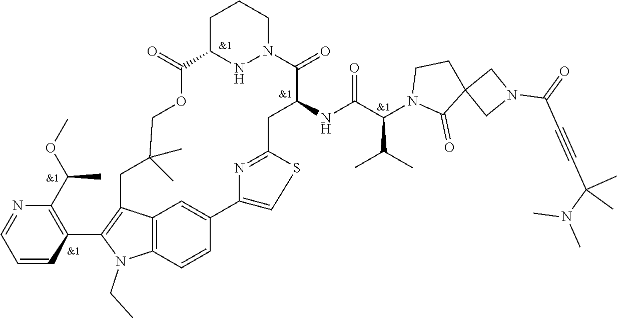 |
| BA324 | 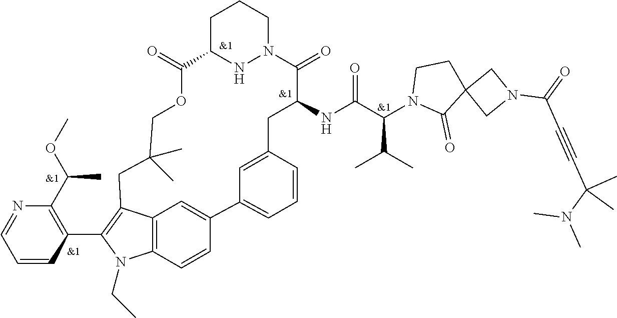 |
| BA325 | 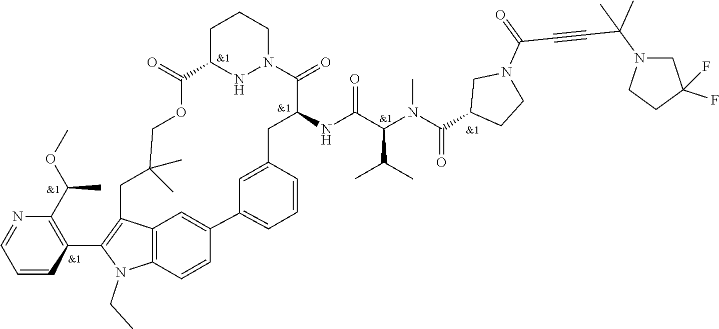 |
| BA326 | 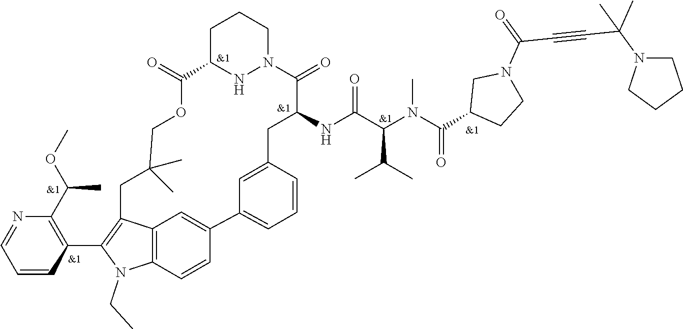 |
| BA327 | 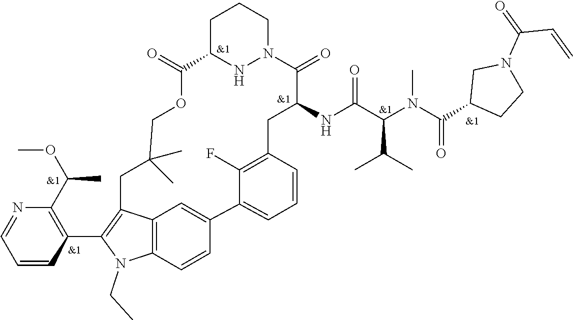 |
| BA328 | 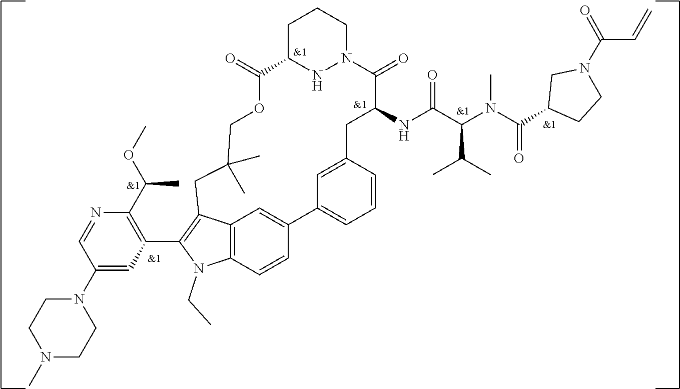 |
| BA329 | 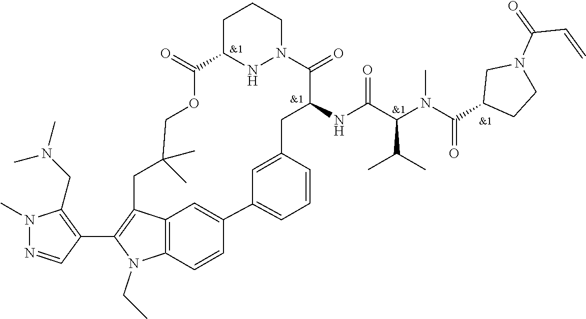 [SMILES: C=CC(=O)N1CC[C@H](C(=O)N(C)[C@H](C(=O)N[C@H]2Cc3cccc(c3)-c3ccc4c(c3)c(c(-c3cnn(C)c3CN(C)C)n4CC)CC(C)(C)COC(=O)[C@@H]3CCCN(N3)C2=O)C(C)C)C1] |
| BA330 | 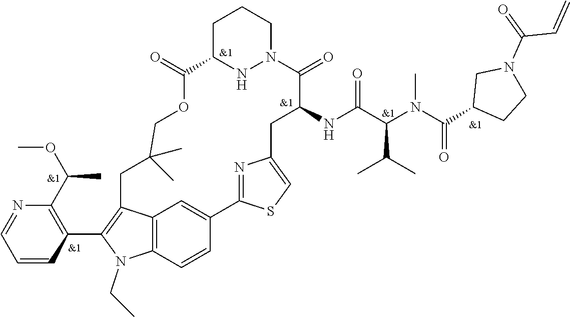 |
| BA331 | 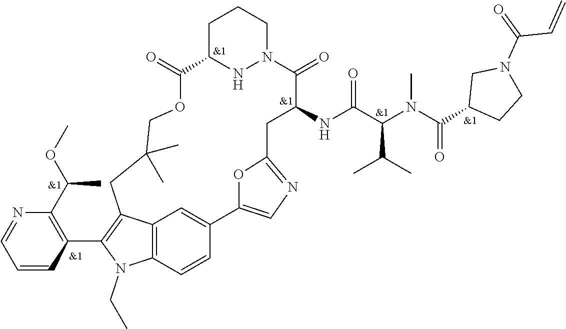 |
| BA332 | 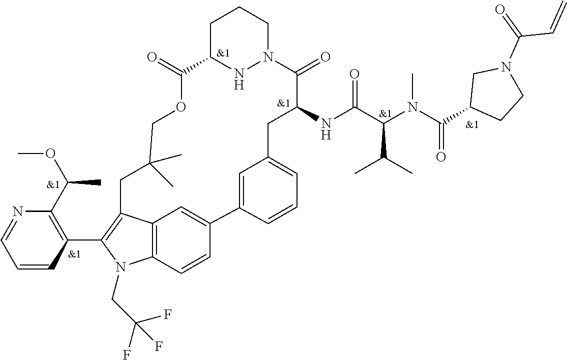 |
| BA333 | 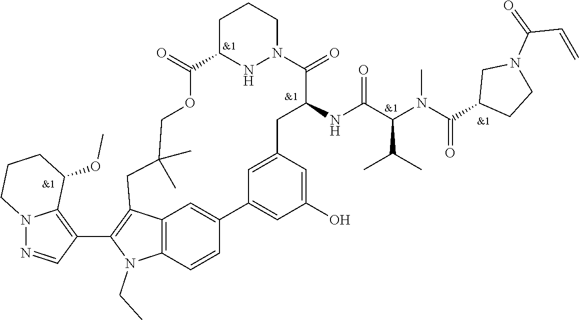 |
| BA334 | 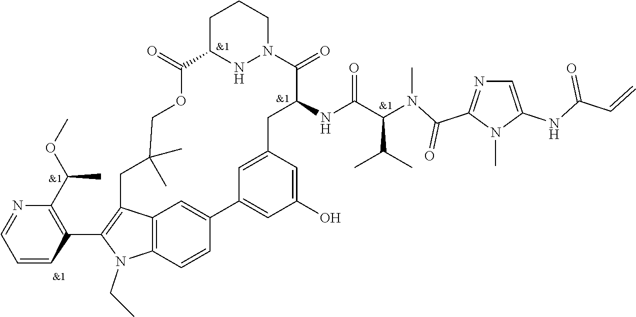 |
| BA335 | 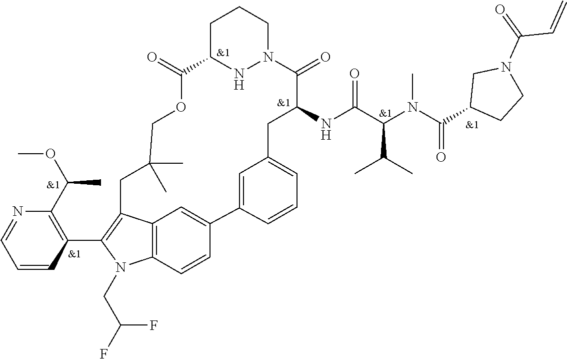 |
| BA336 | 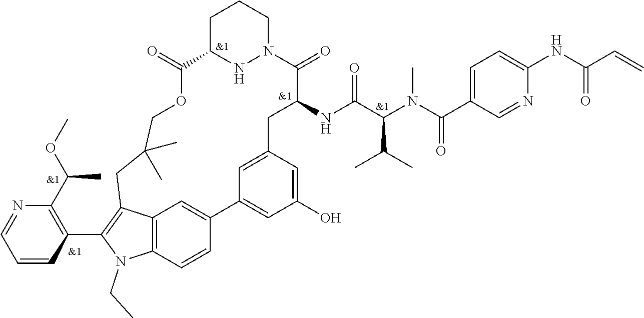 |
| BA337 | 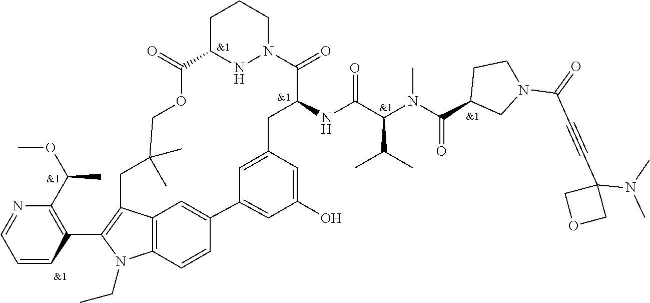 |
| BA338 | 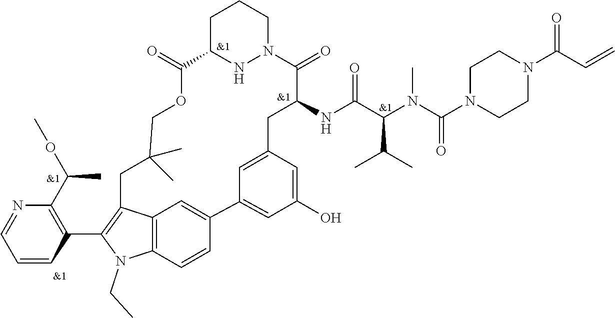 |
| BA339 | 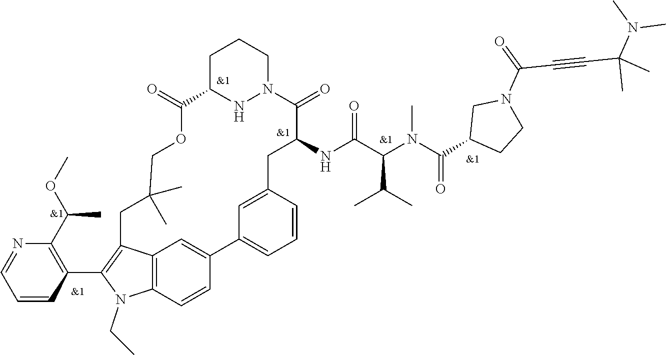 |
| BA340 | 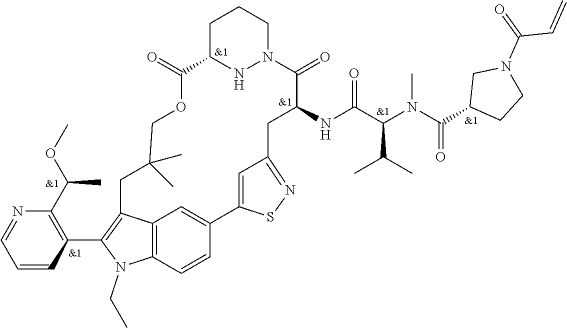 |
| BA341 | 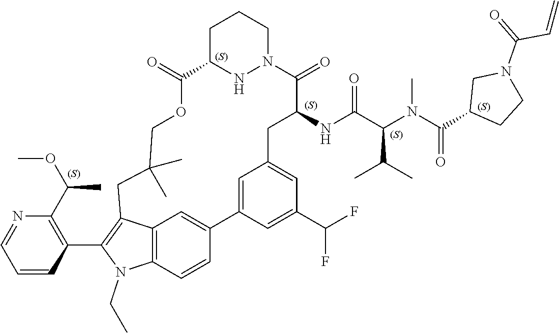 |
| BA342 | 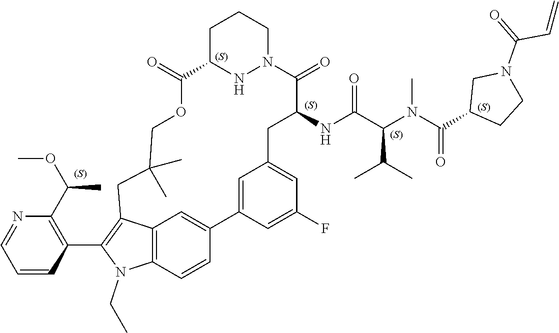 |
| BA343 | 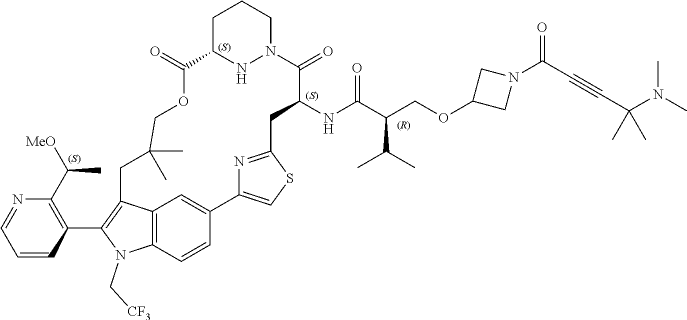 |
| BA344 | 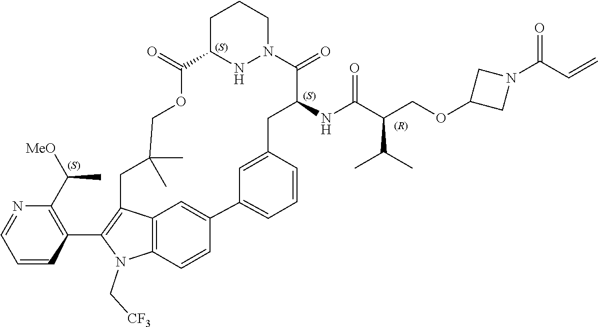 |
| BA345 | 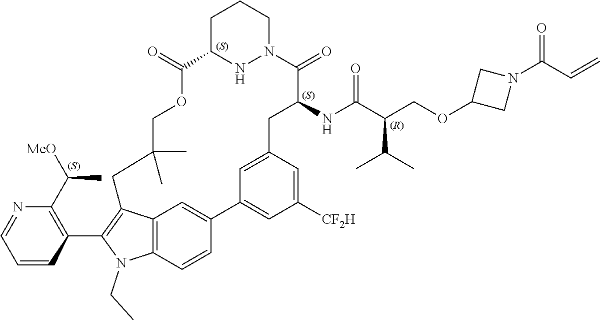 |
| BA346 | 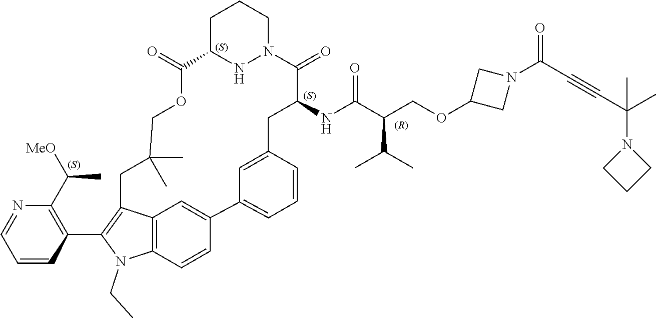 |
| BA347 | 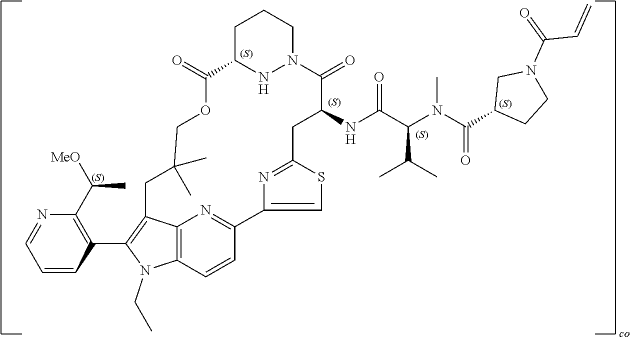 |
| BA348 | 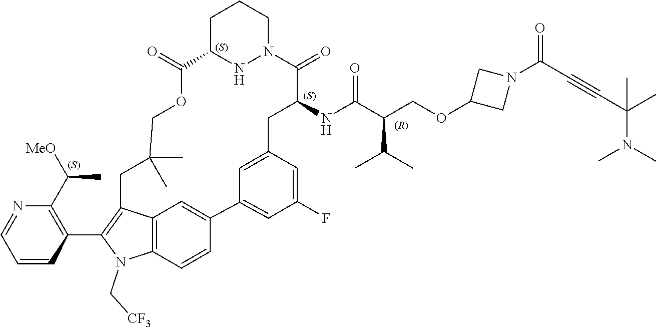 |
| BA349 | 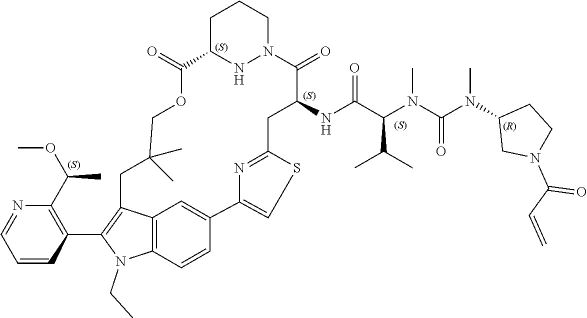 |
| BA350 | 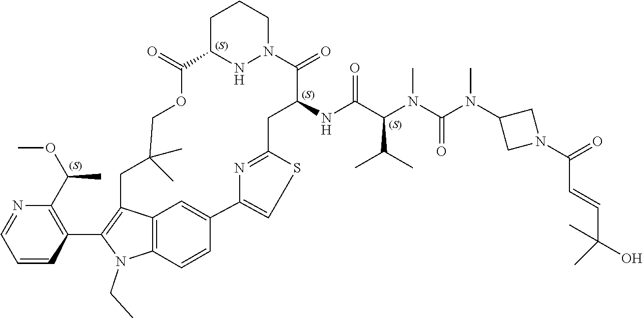 |
| BA351 | 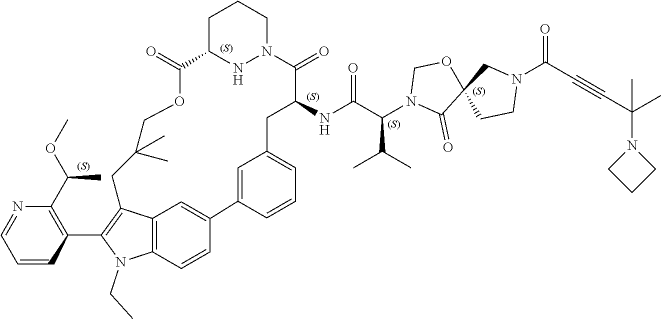 |
| BA352 | 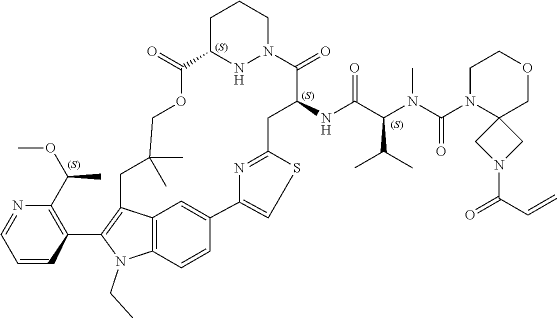 |
| BA353 | 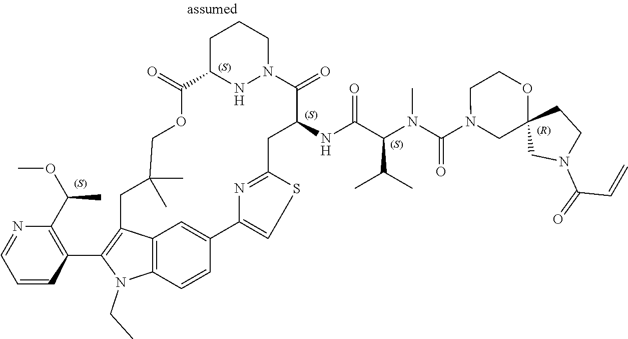 |
| BA354 | 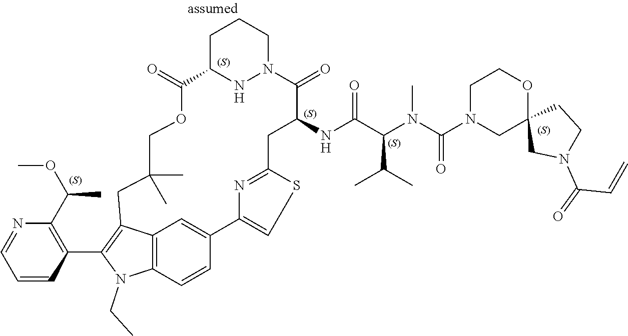 |
| BA355 | 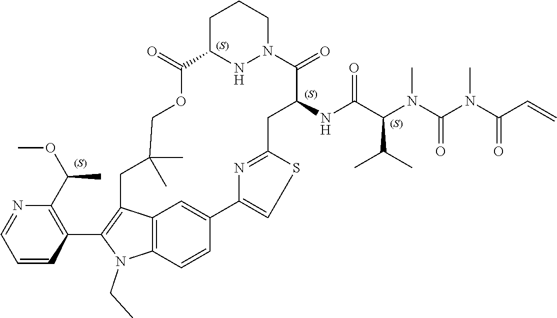 |
| BA356 | 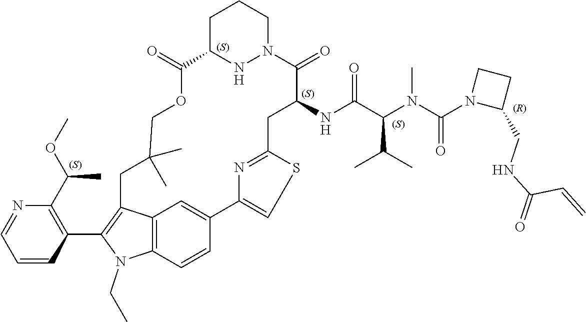 |
| BA357 | 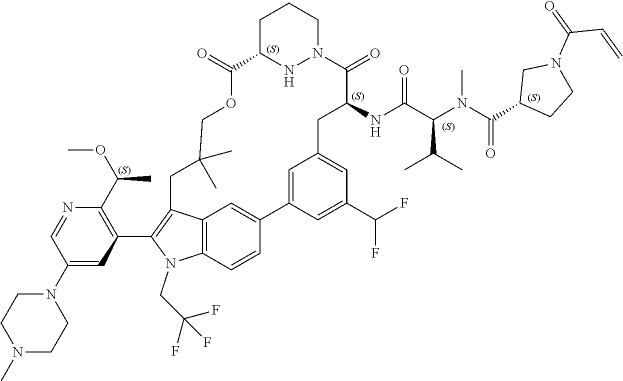 |
| BA358 | 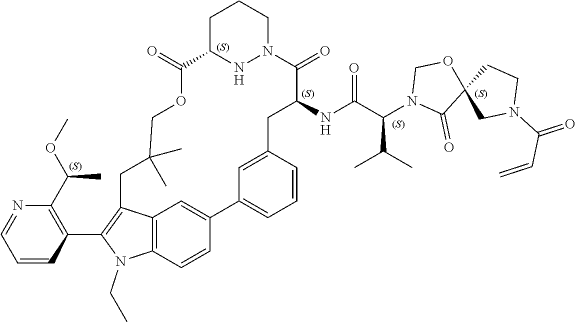 |
| BA359 | 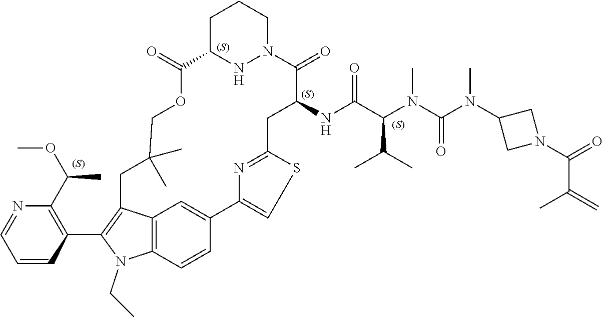 |
| BA360 | 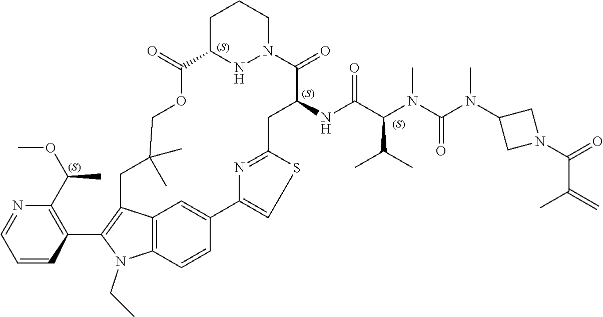 |
| BA361 | 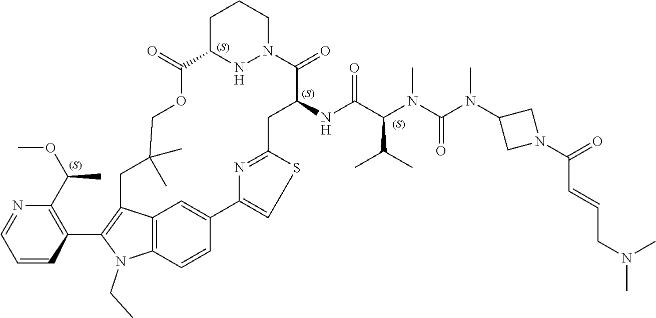 |
| BA362 | 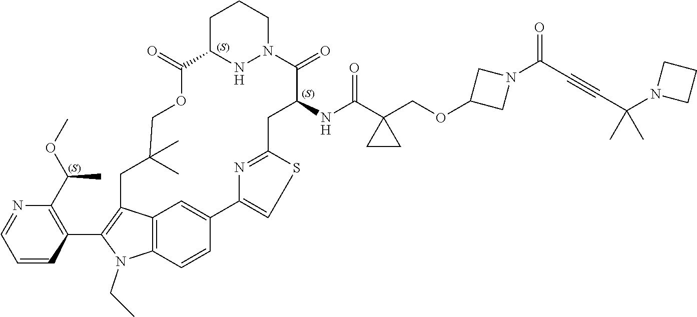 |
| BA363 | 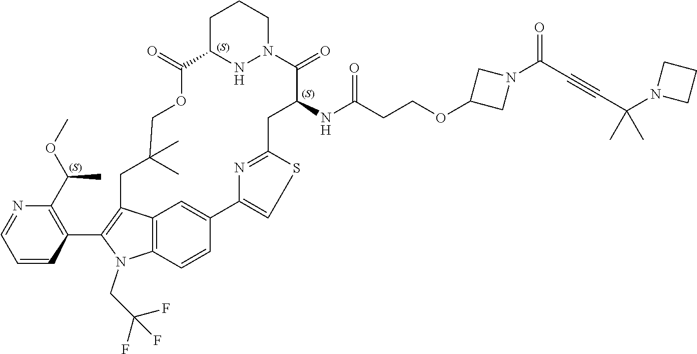 |
| BA364 | 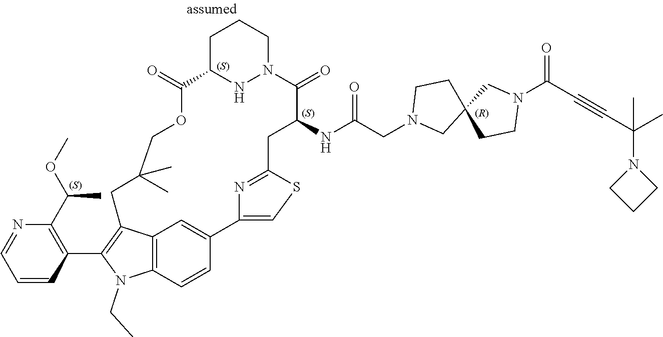 |
| BA365 | 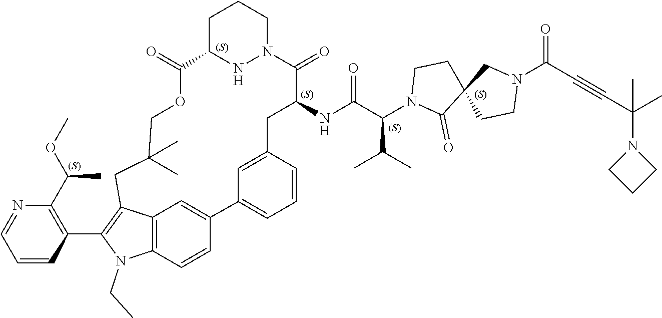 |
| BA366 | 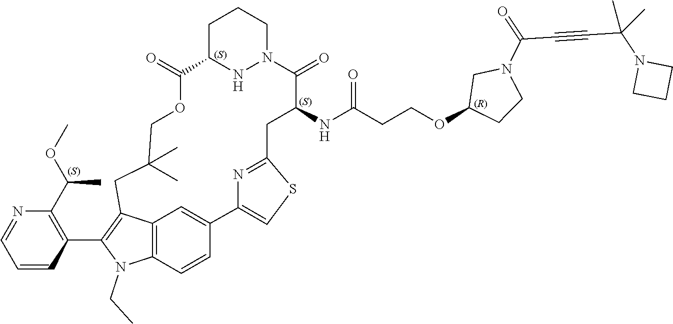 |
| BA367 | 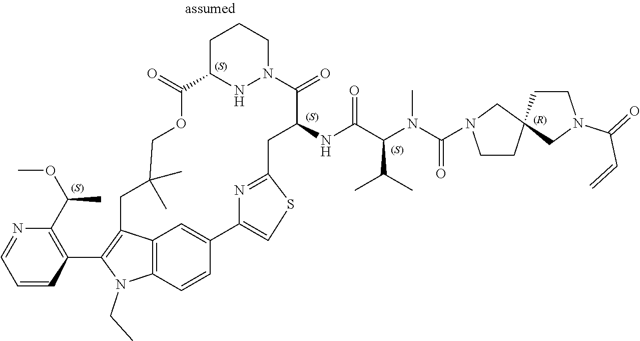 |
| BA368 | 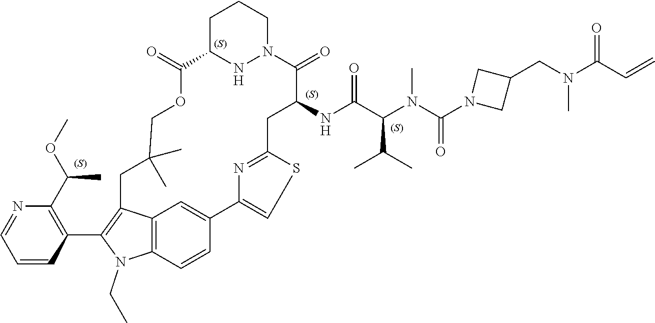 |
| BA369 | 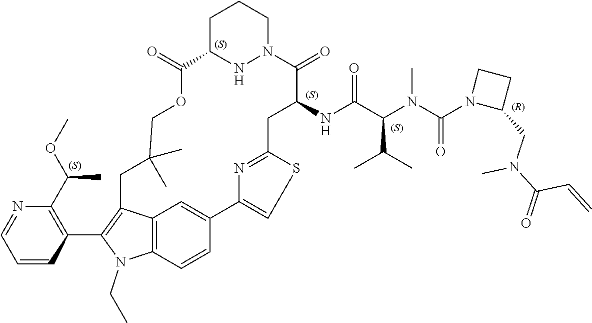 |
| BA370 | 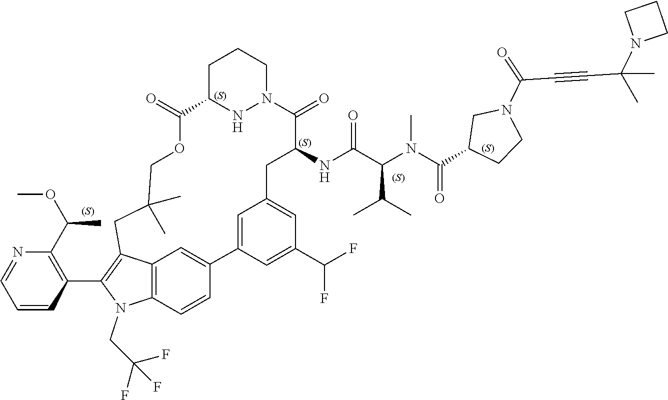 |
| BA371 | 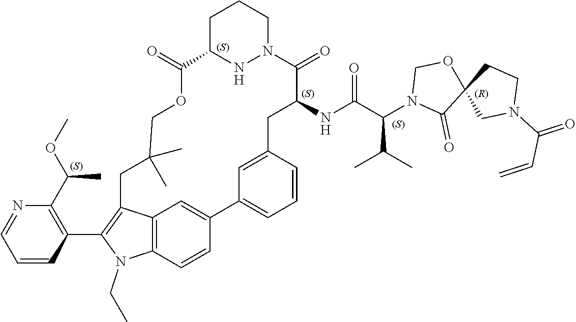 |
| BA372 | 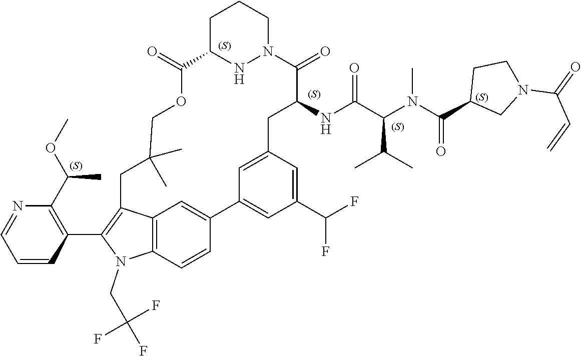 |
| BA373 | 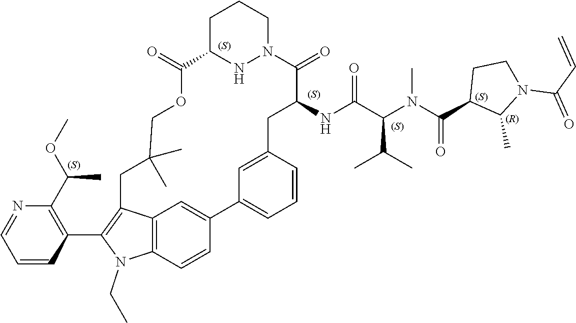 |
| BA374 | 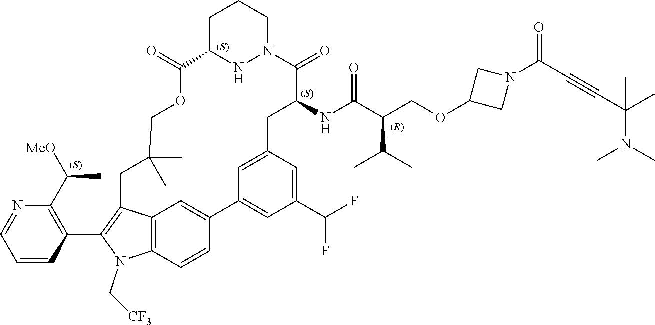 |
| BA375 | 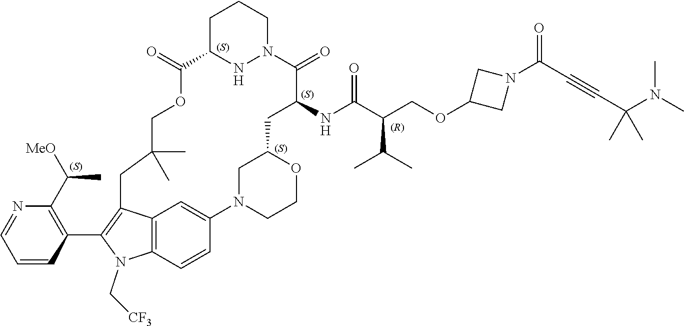 |
| BA376 | 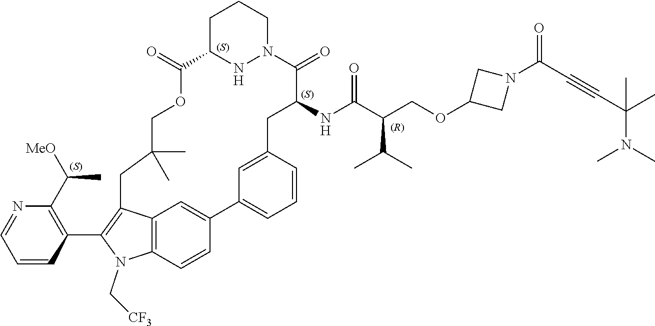 |
| BA377 | 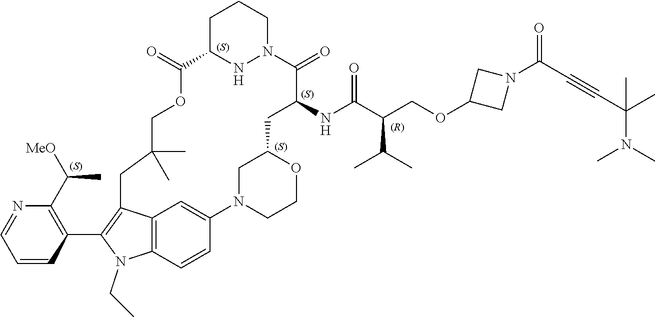 |
| BA378 | 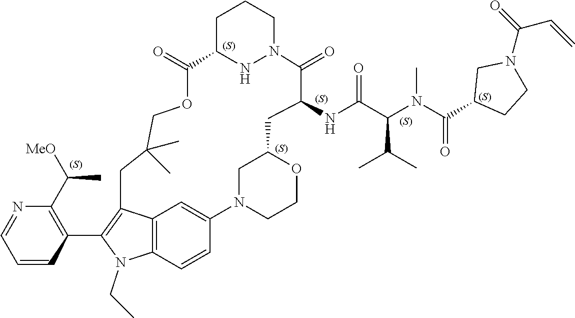 |
| BA379 | 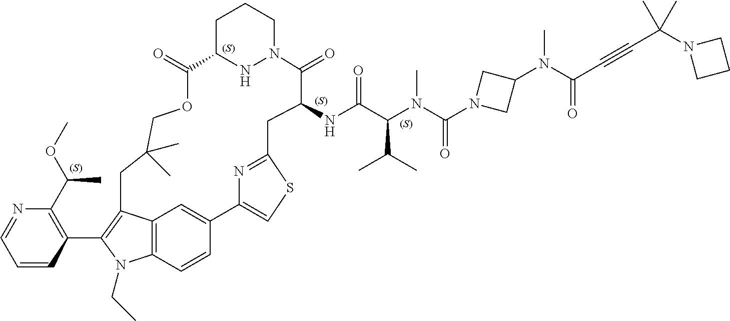 |
| BA380 | 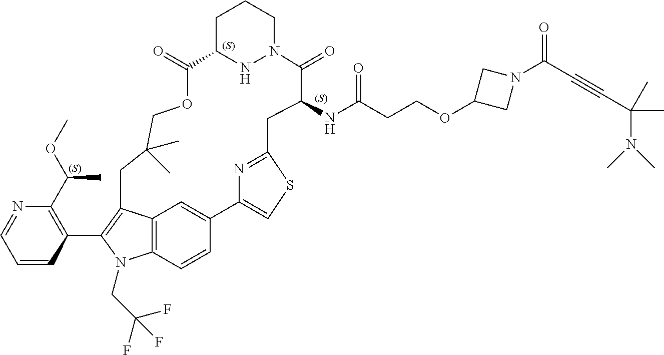 |
| BA381 | 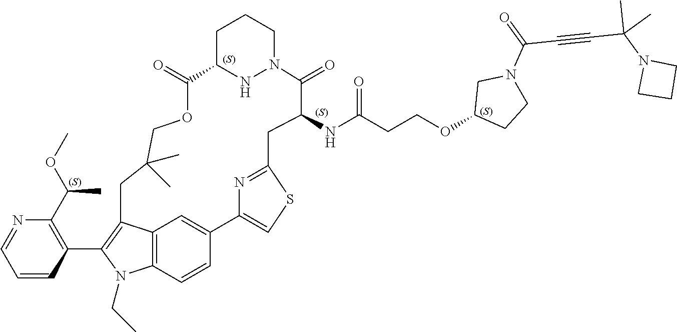 |
| BA382 | 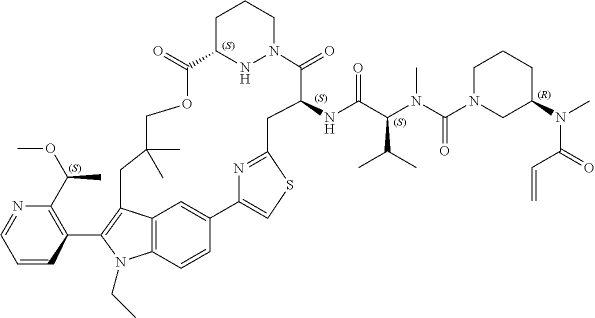 |
| BA383 | 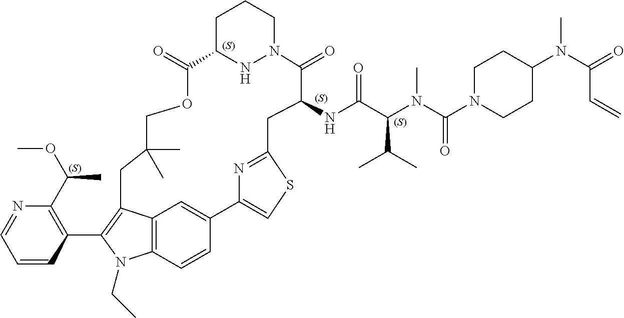 |
| BA384 | 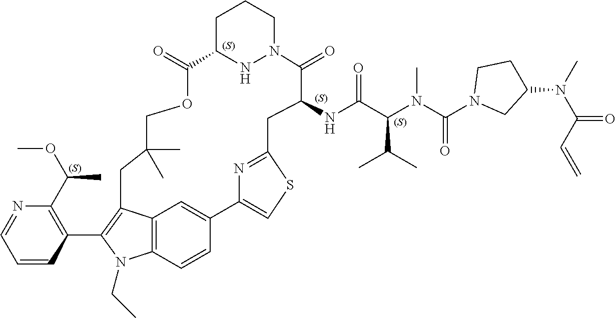 |
| BA385 | 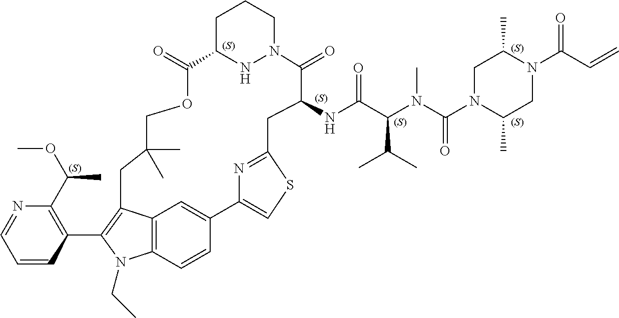 |
| BA386 | 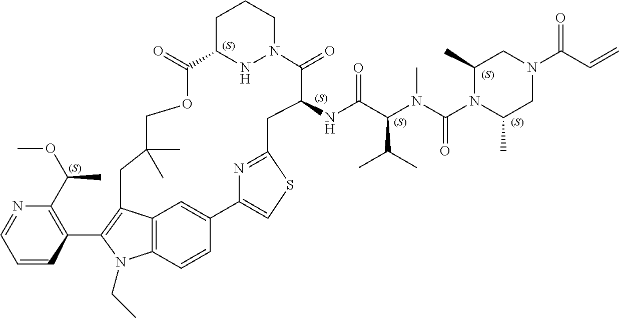 |
| BA387 | 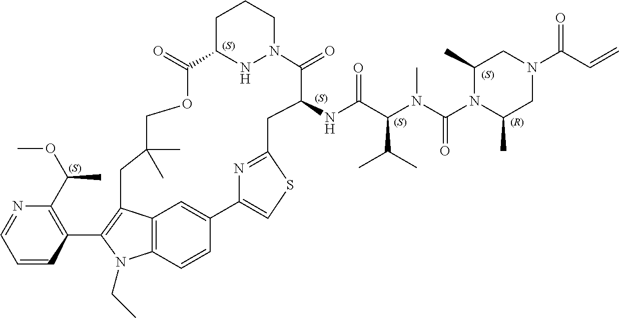 |
| BA388 | 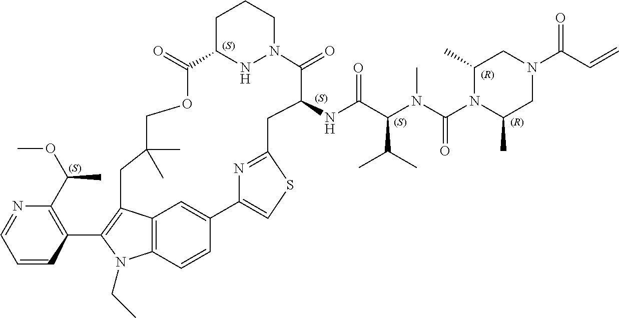 |
| BA389 | 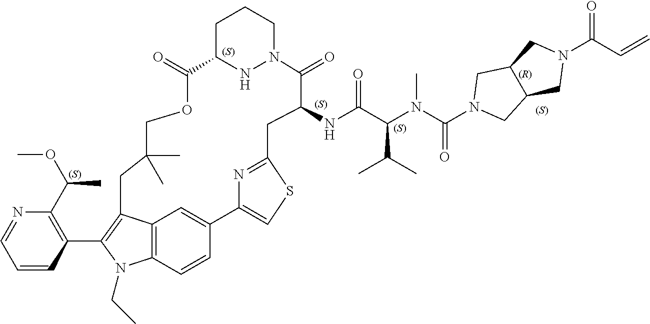 |
| BA390 | 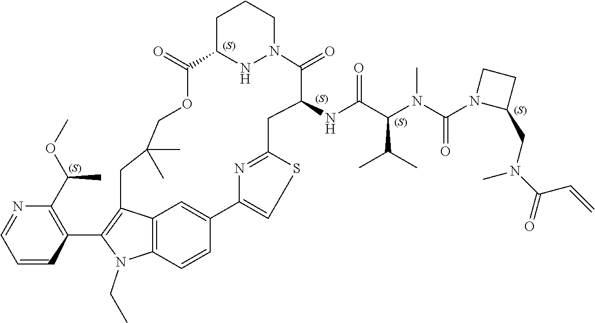 |
| BA391 | 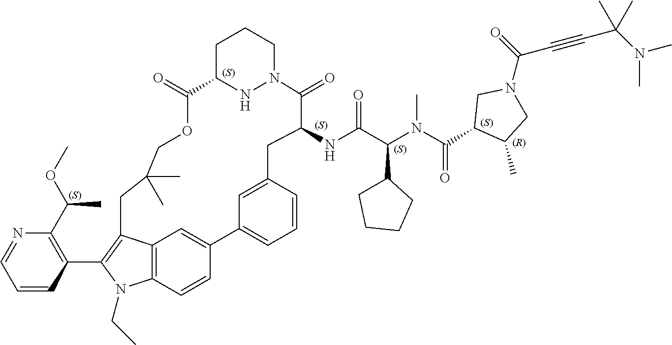 |
| BA392 | 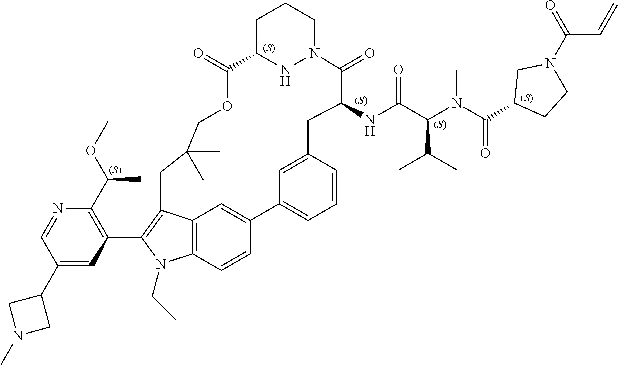 |
| BA393 | 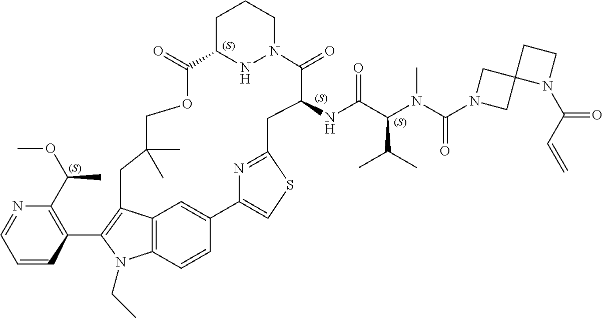 |
| BA394 | 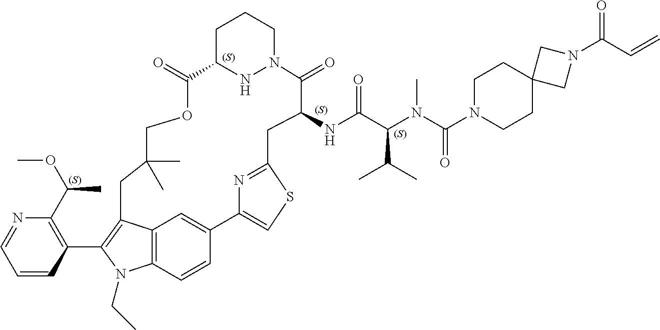 |
| BA395 | 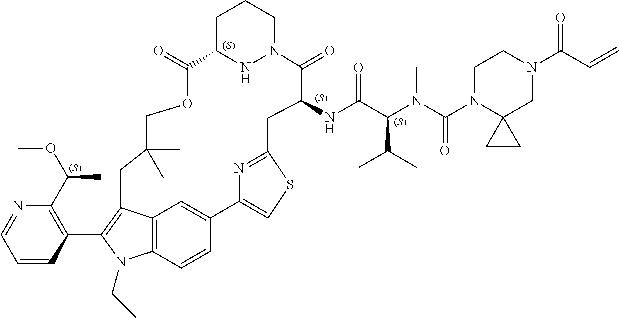 |
| BA396 | 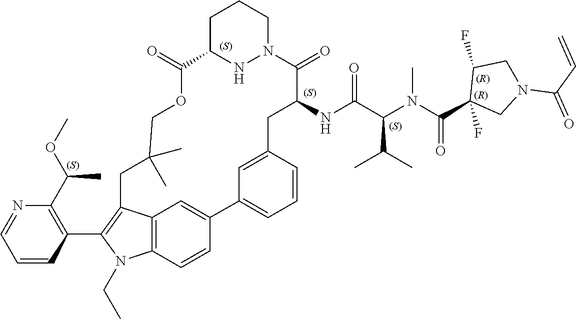 |
| BA397 | 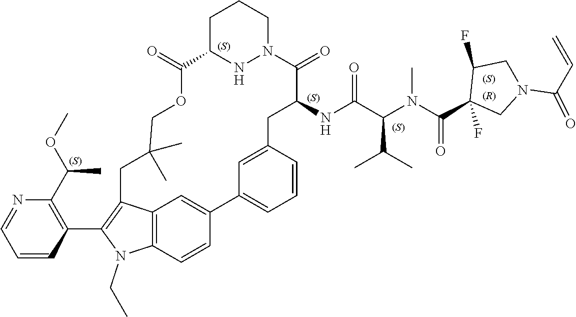 |
| BA398 | 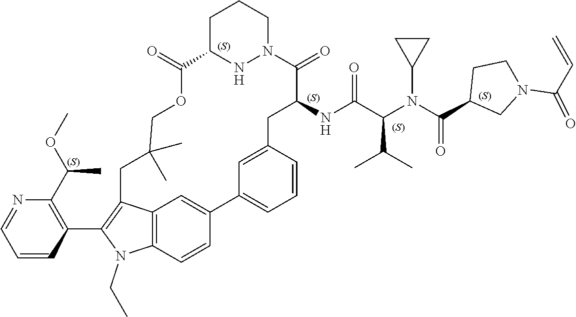 |
| BA399 | 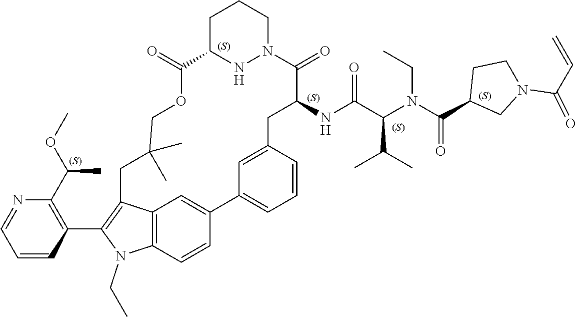 |
| BA400 | 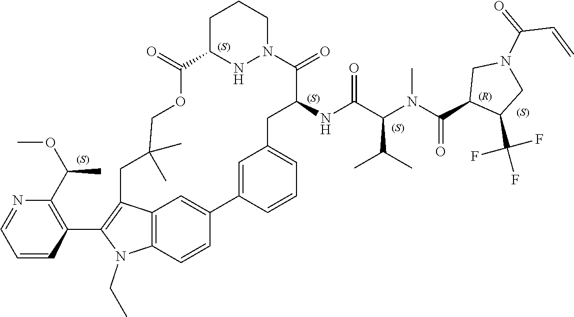 |
| BA401 | 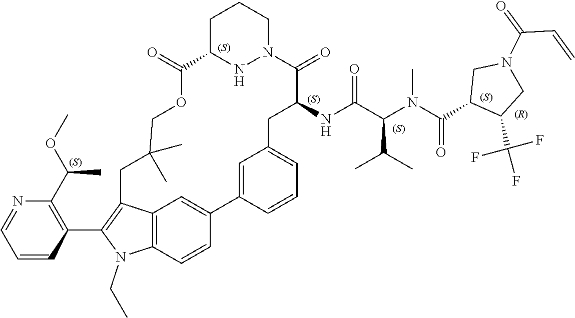 |
| BA402 | 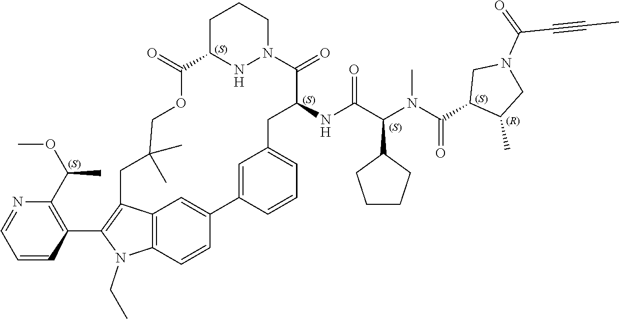 |
| BA403 | 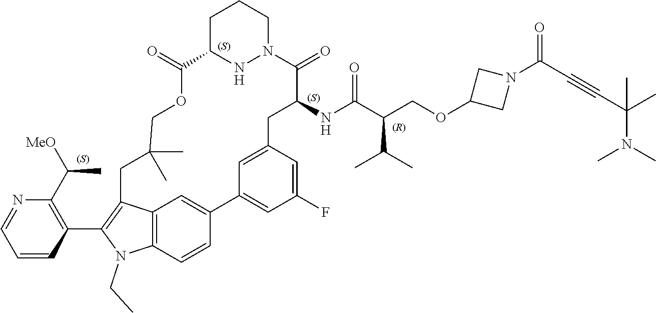 |
| BA404 | 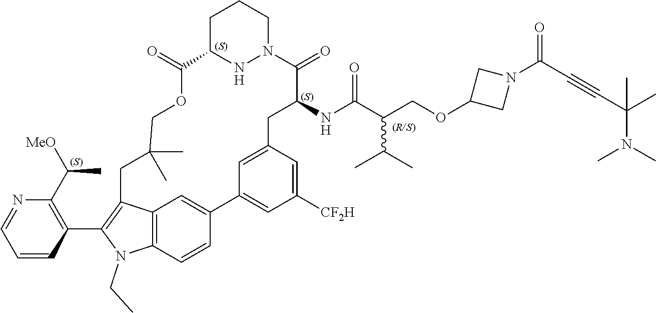 |
| BA405 | 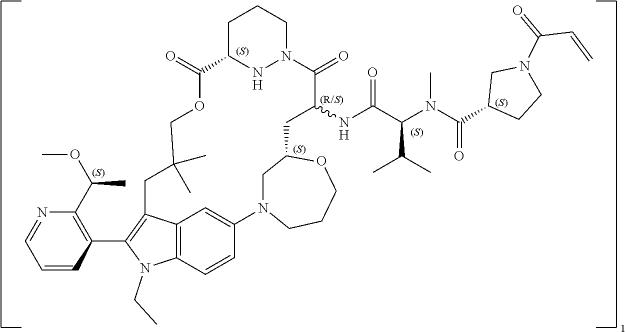 |
| BA406 | 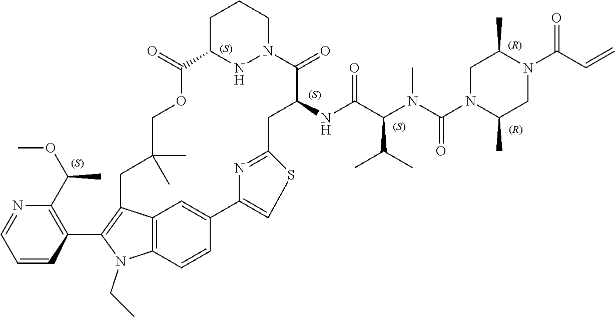 |
| BA407 | 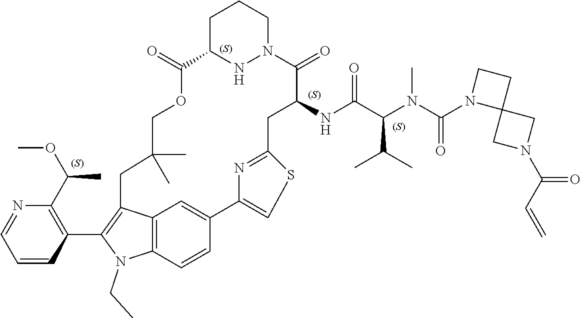 |
| BA408 | 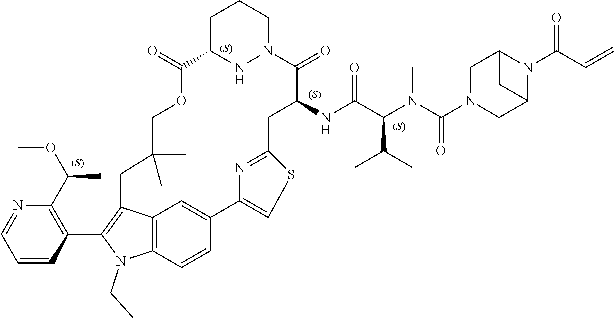 |
| BA409 | 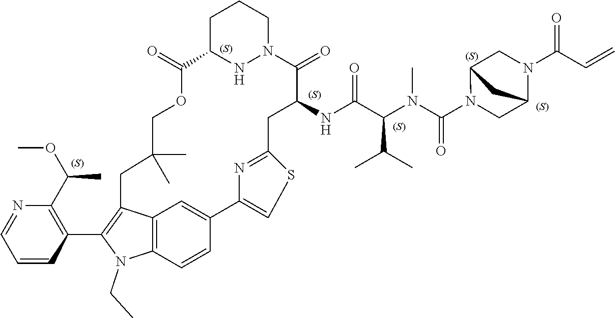 |
| BA410 | 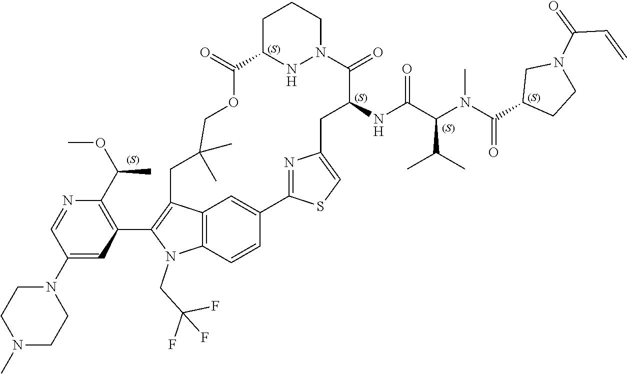 |
| BA411 | 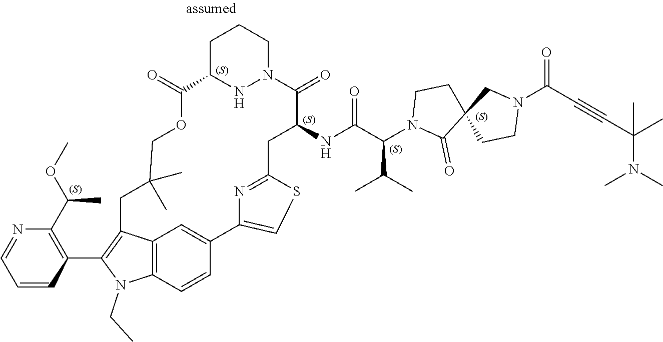 |
| BA412 | 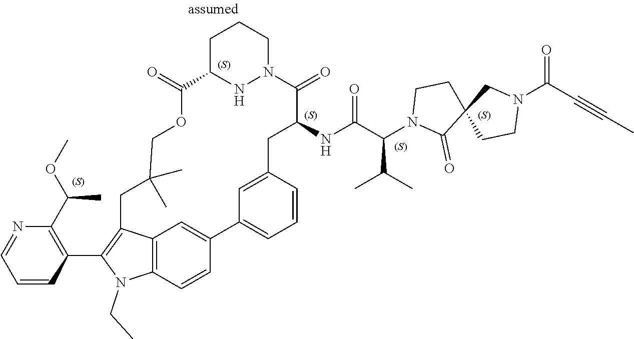 |
| BA413 | 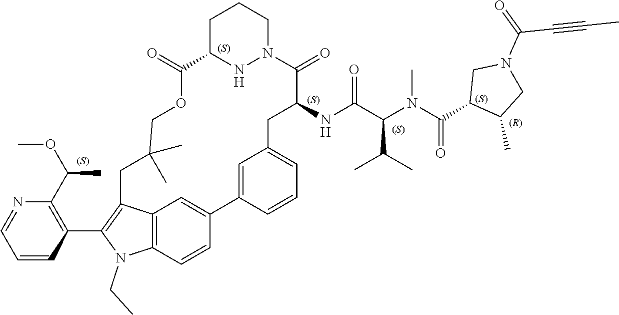 |
| BA414 | 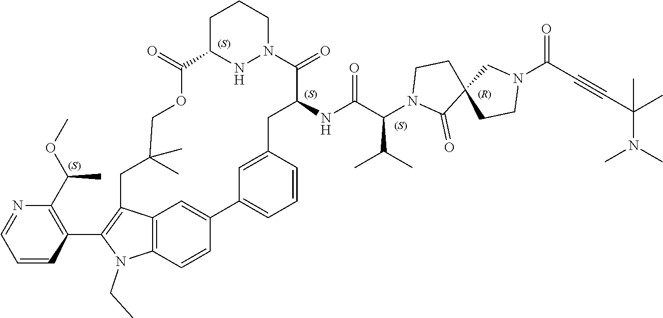 |
| BA415 | 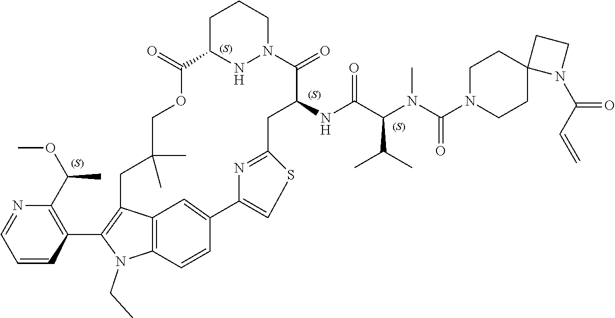 |
| BA416 | 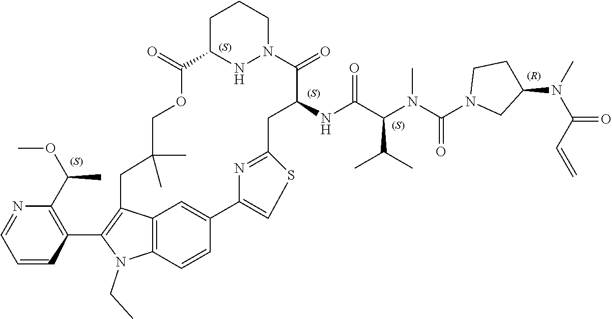 |
| BA417 | 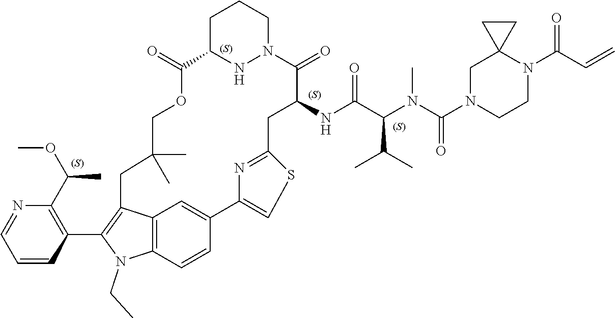 |
| BA418 | 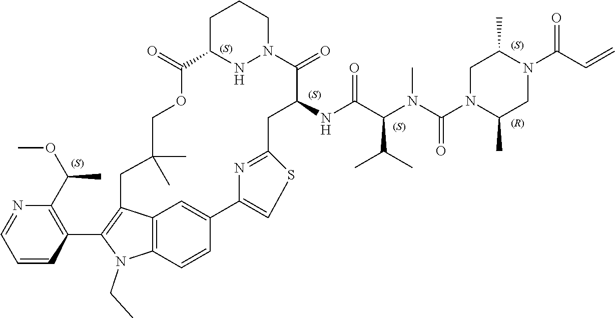 |
| BA419 | 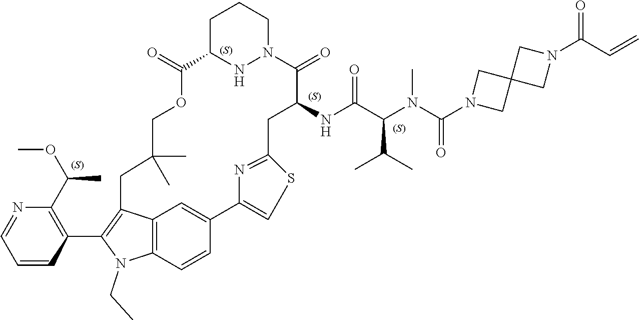 |
| BA420 | 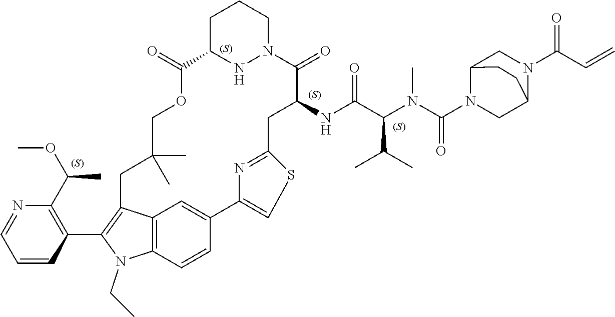 |
| BA421 | 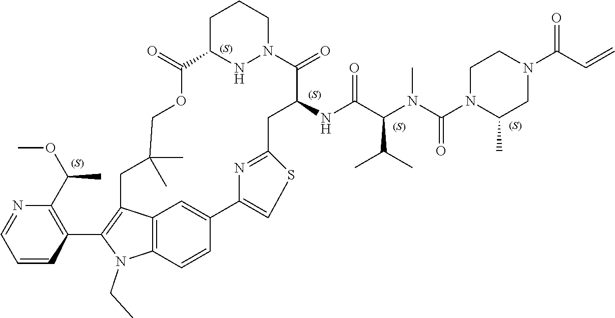 |
| BA422 | 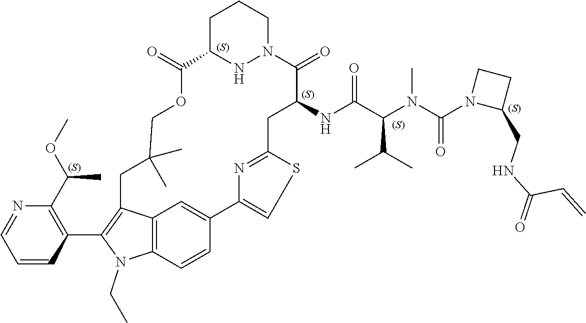 |
| BA423 | 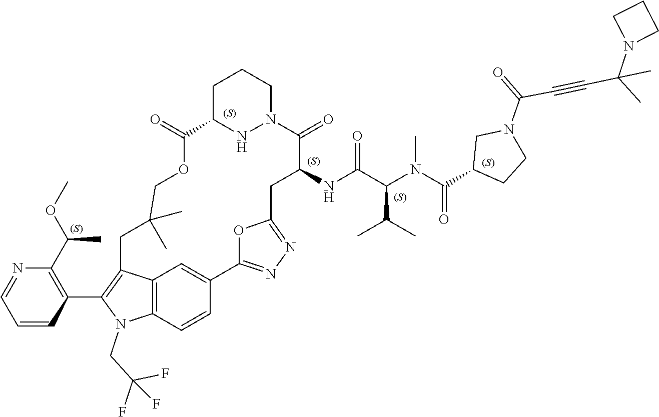 |
| BA424 | 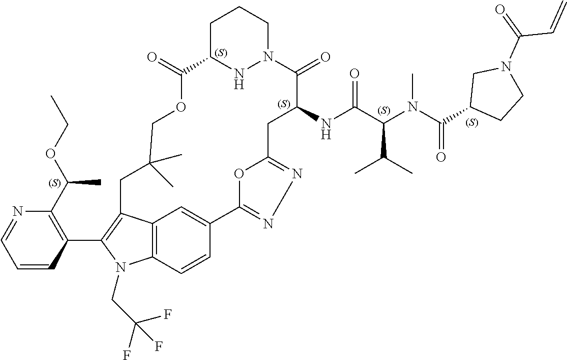 |
| BA425 | 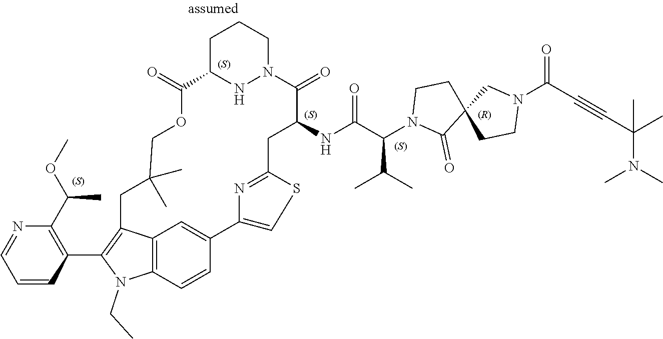 |
| BA426 | 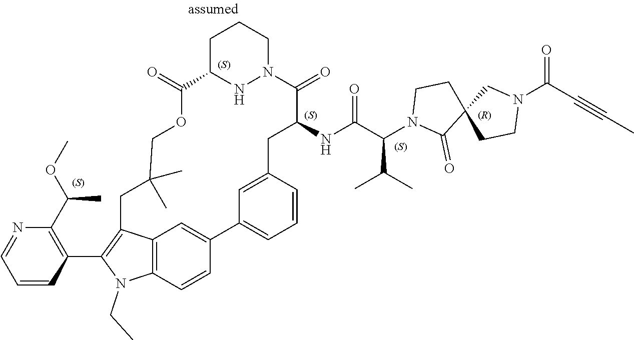 |
| BA427 | 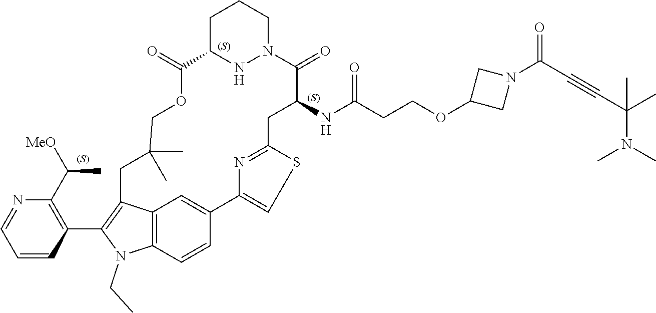 |
| BA428 | 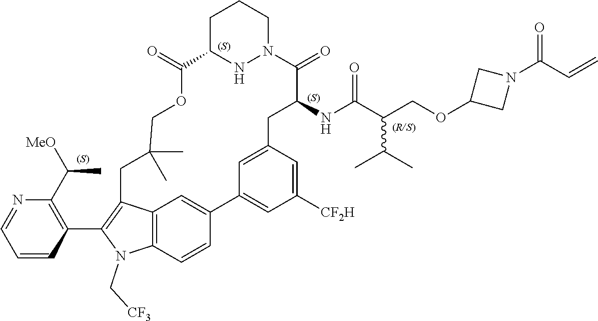 |
| BA429 | 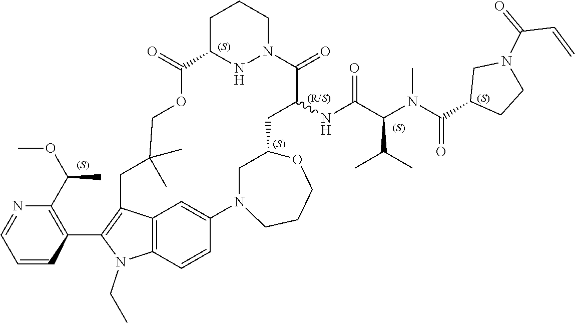 |
| BA430 | 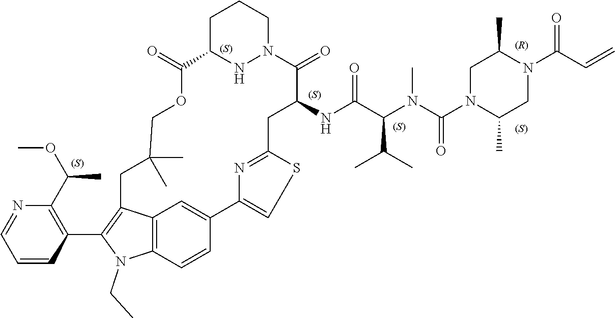 |
| BA431 | 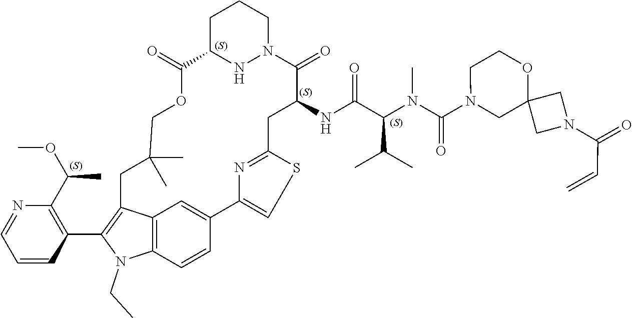 |
| BA432 | 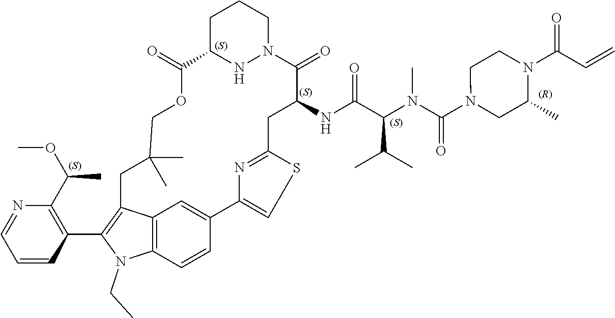 |
| BA433 | 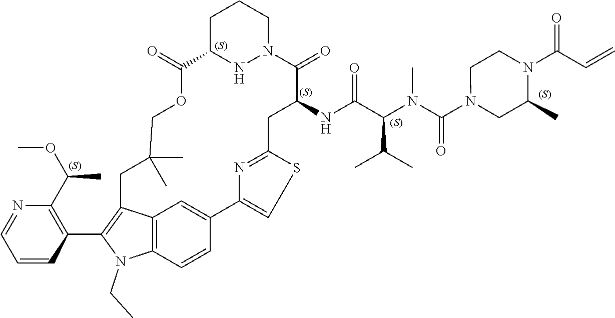 |
| BA334 | 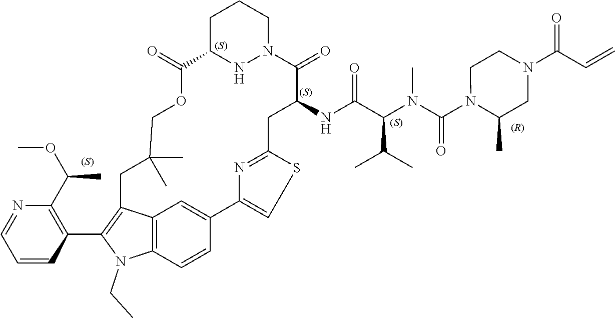 |
| BA435 | 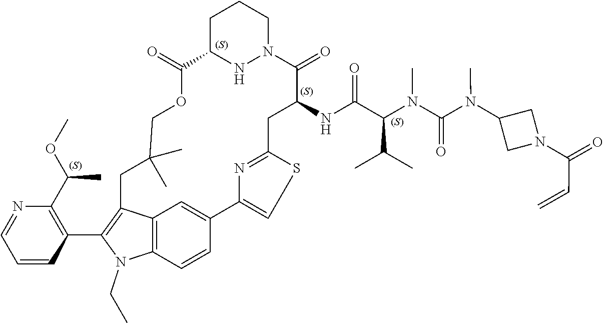 |
| BA436 | 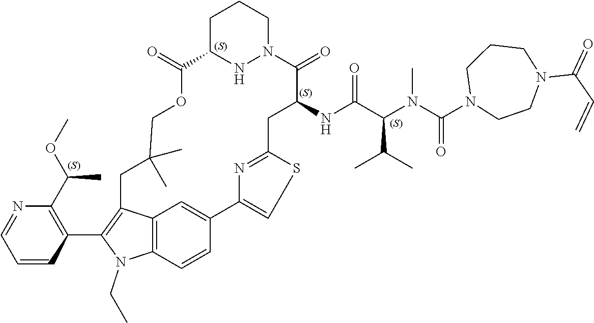 |
| BA437 | 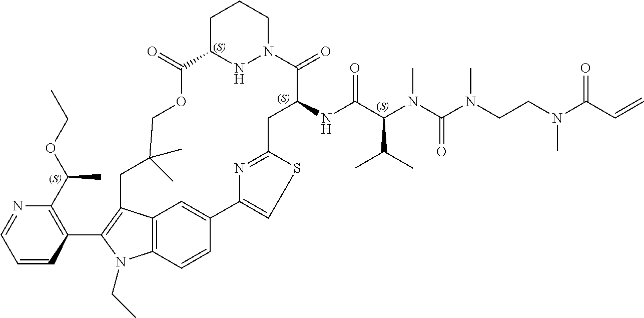 |
| BA438 | 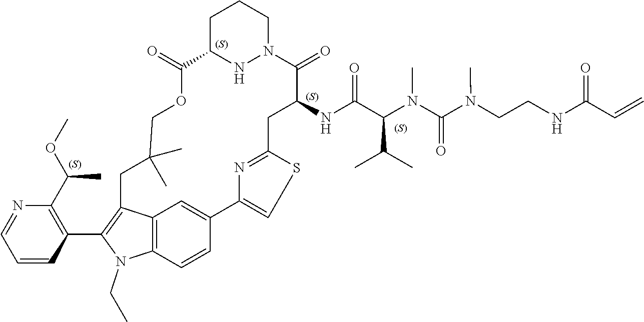 |
| BA439 | 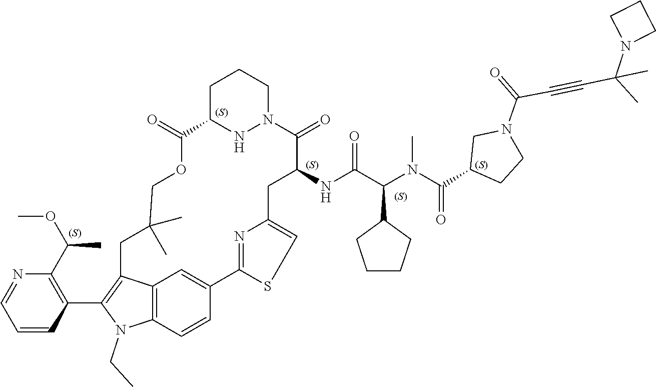 |
| BA440 | 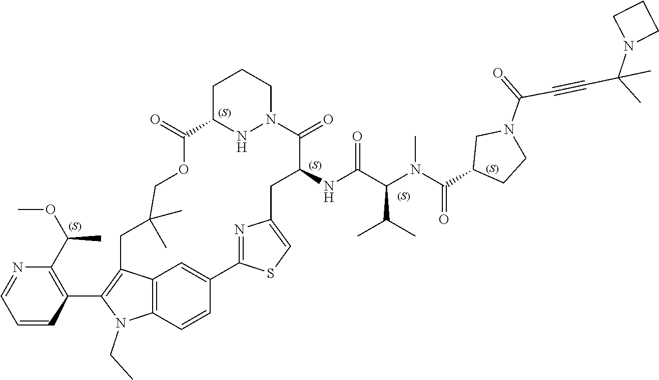 |
| BA441 | 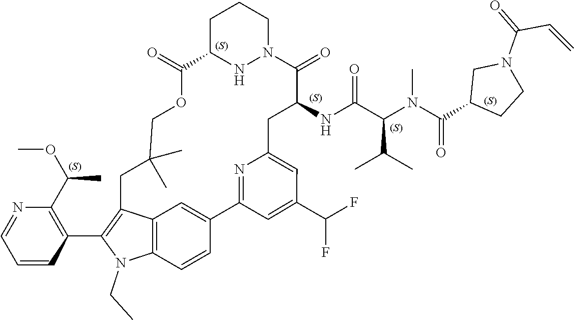 |
| BA442 | 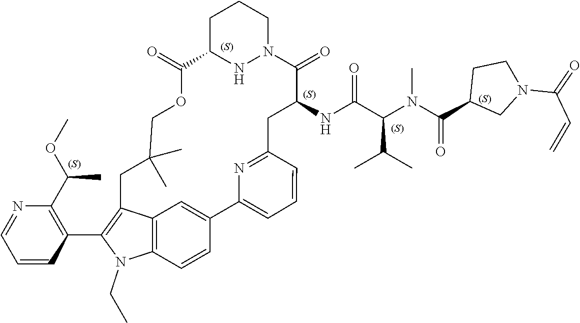 |
| BA443 | 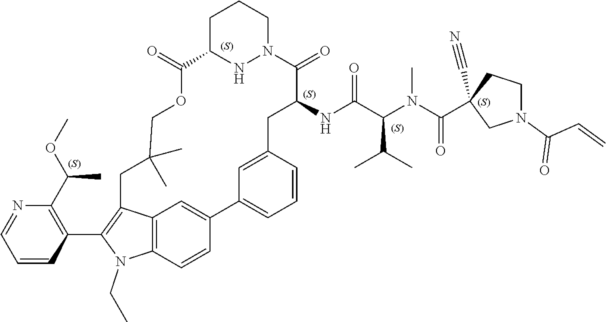 |
| BA444 | 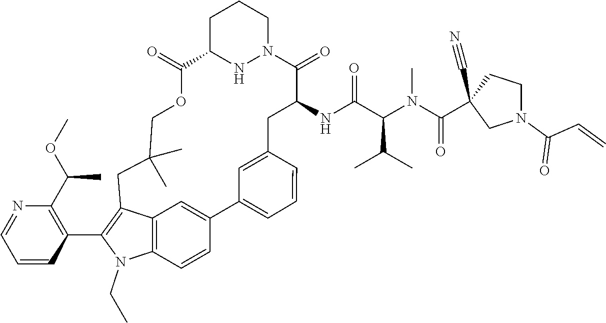 |
| BA445 | 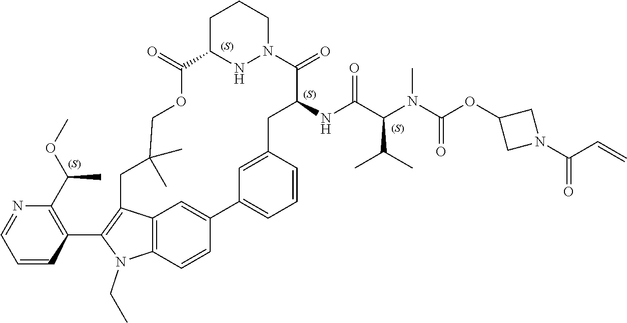 |
| BA446 | 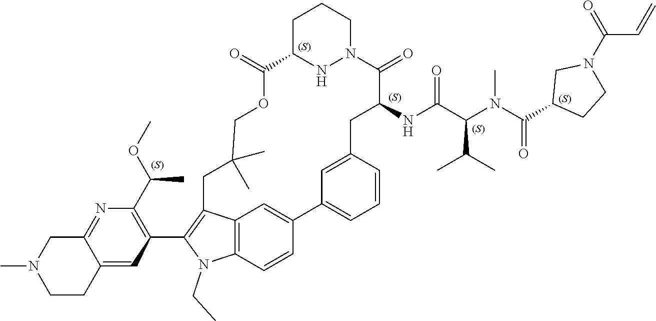 |
| BA447 | 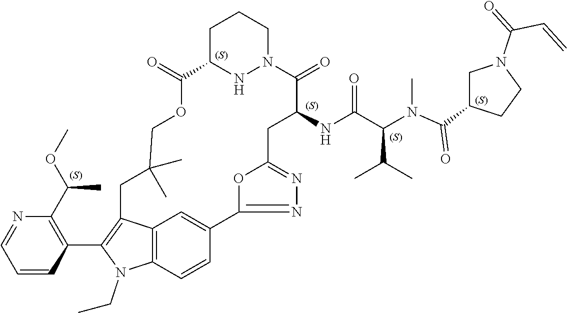 |
| BA448 | 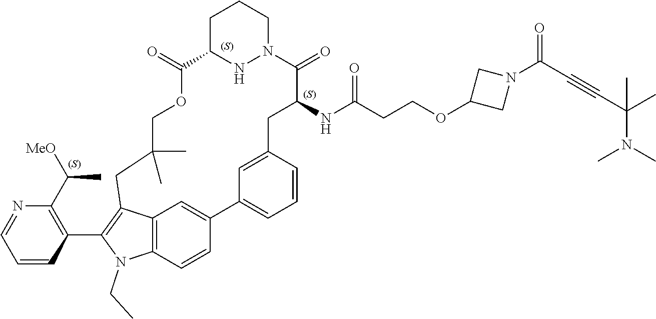 |
| BA449 | 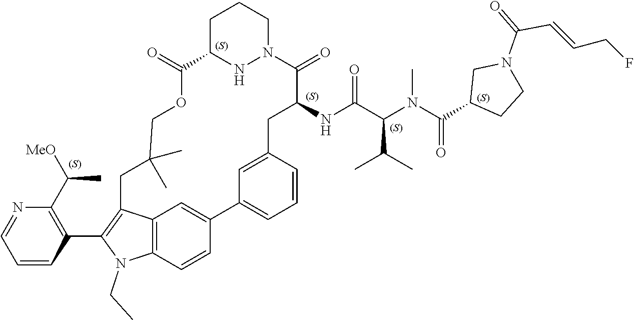 |
| BA450 | 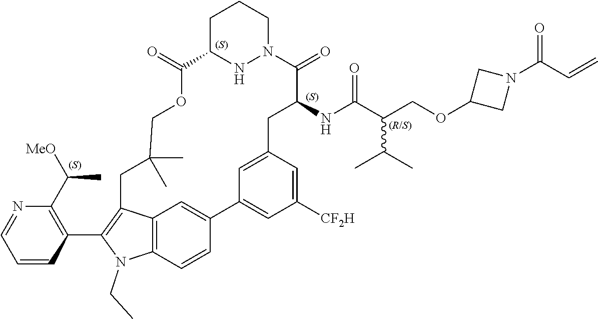 |
| BA451 | 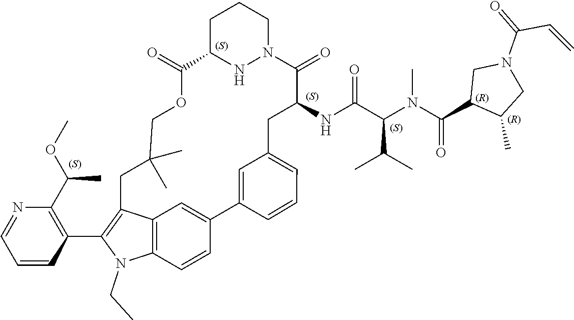 |
| BA452 | 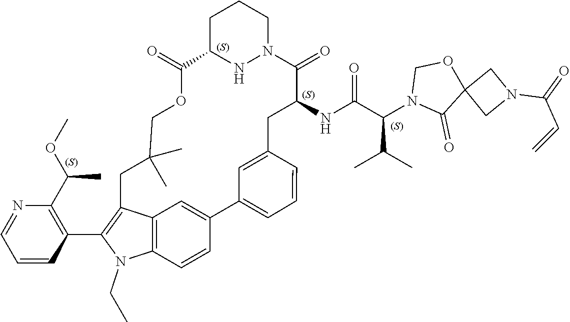 |
| BA453 | 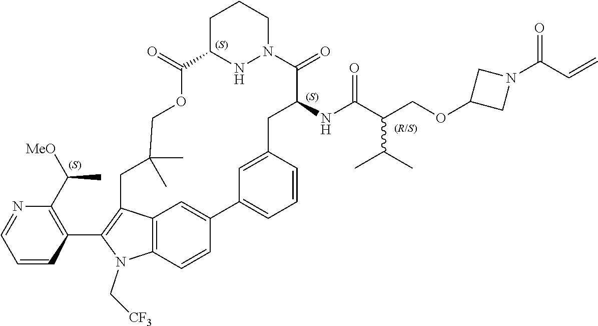 |
| BA454 | 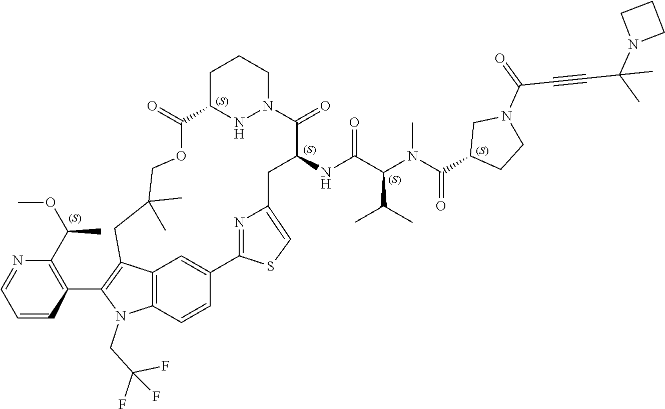 |
| BA455 | 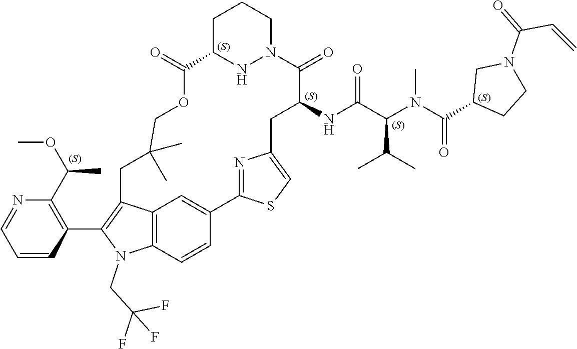 |
| BA456 | 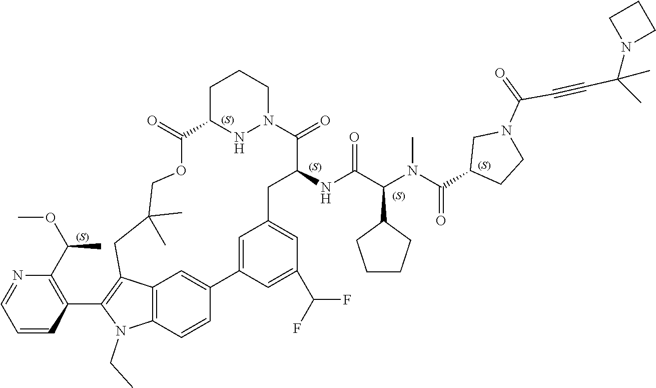 |
| BA457 | 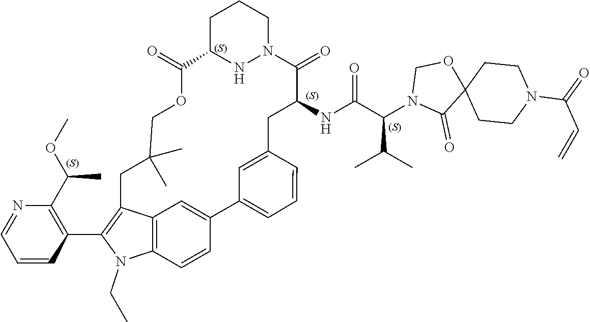 |
| BA458 | 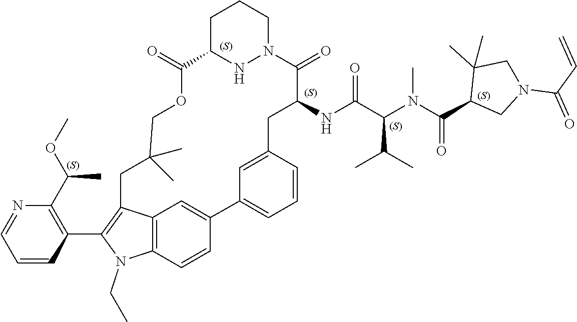 |
| BA459 | 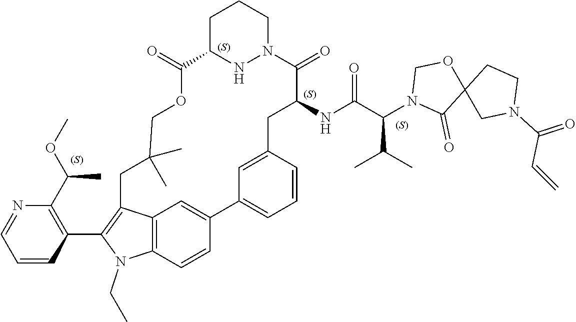 |
| BA460 | 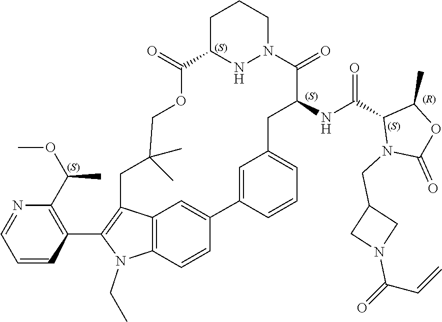 |
| BA461 | 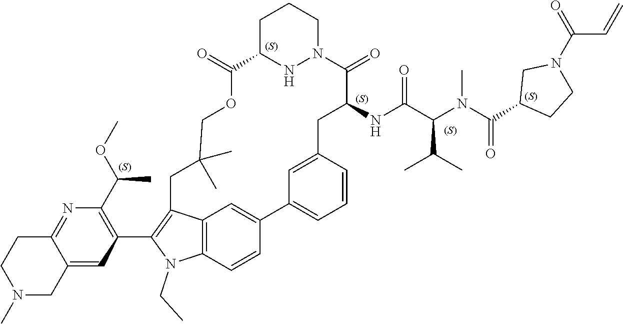 |
| BA462 | 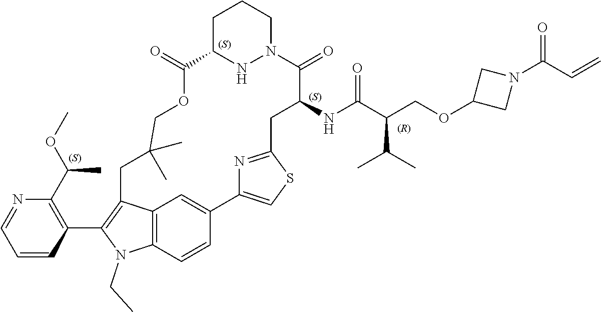 |
| BA463 | 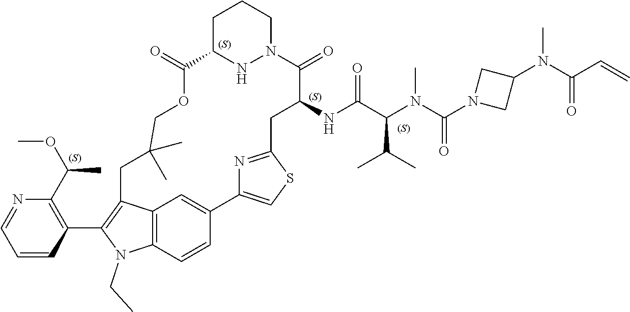 |
| BA464 | 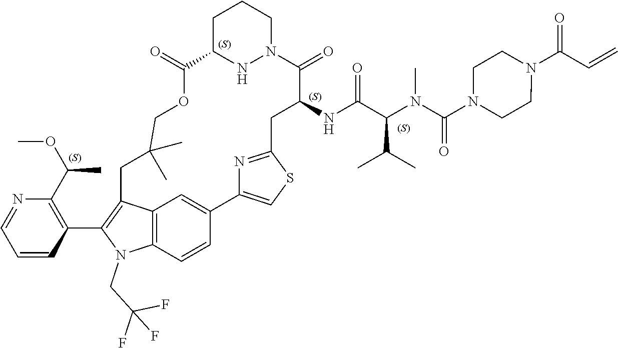 |
| BA465 | 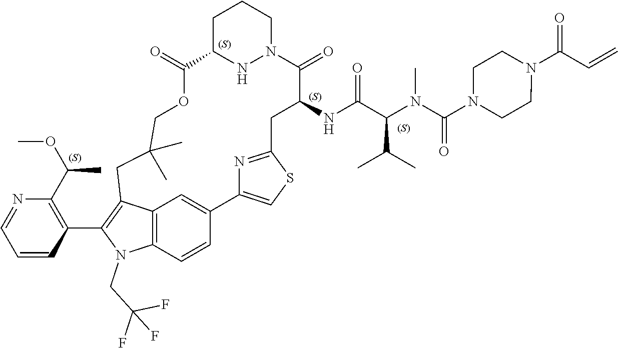 |
| BA466 | 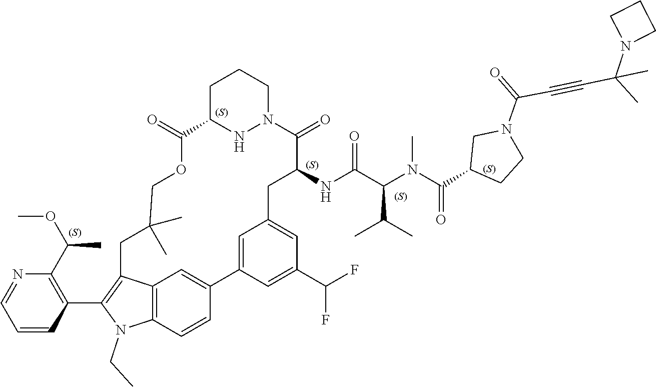 |
| BA467 | 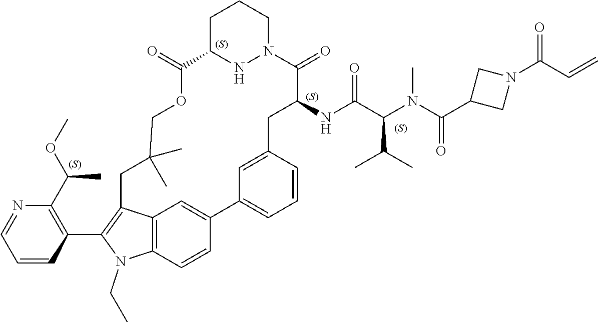 |
| BA468 | 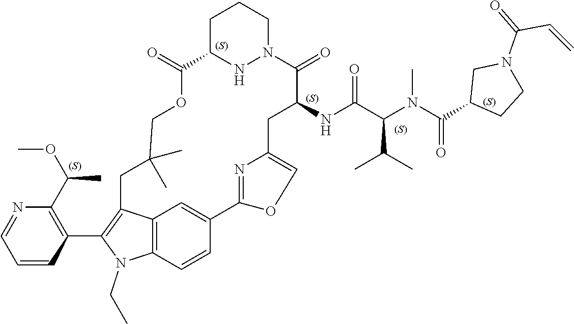 |
| BA469 | 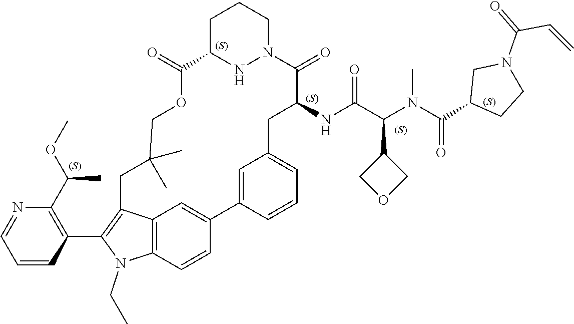 |
| BA470 | 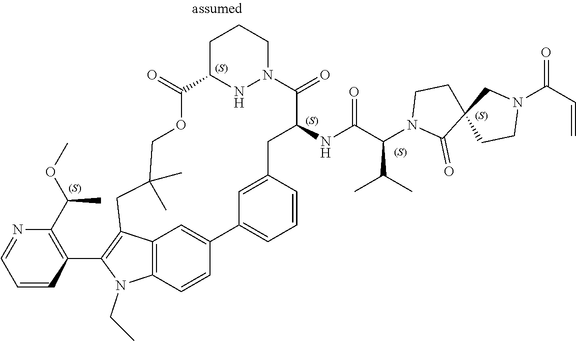 |
| BA471 | 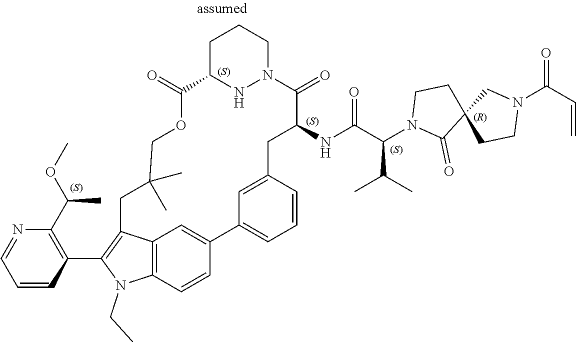 |
| BA472 | 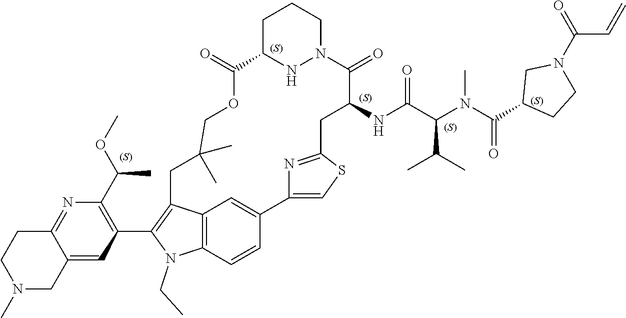 |
| BA473 | 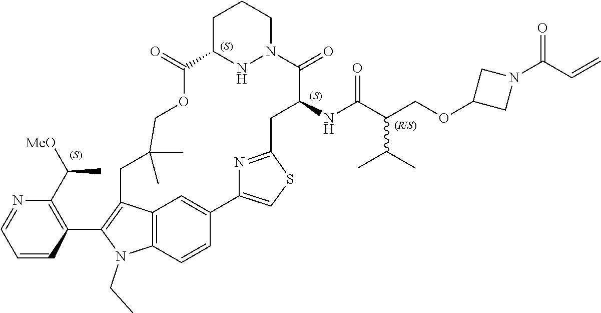 |
| BA474 | 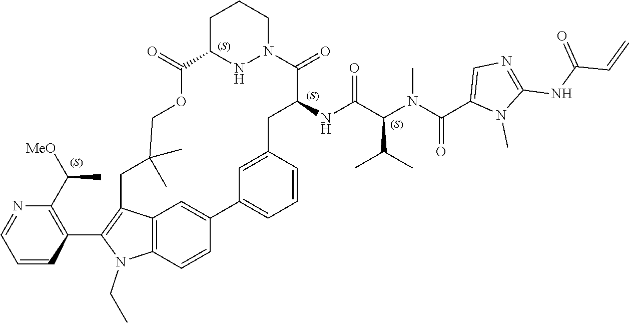 |
| BA475 | 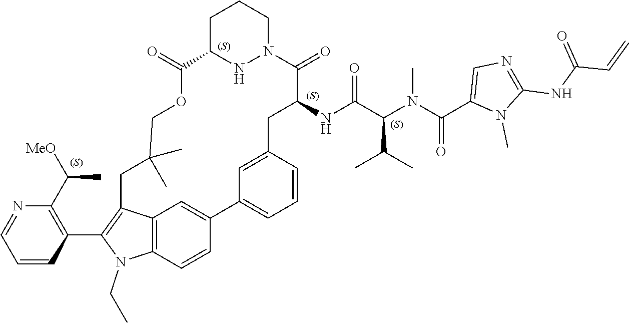 |
| BA476 | 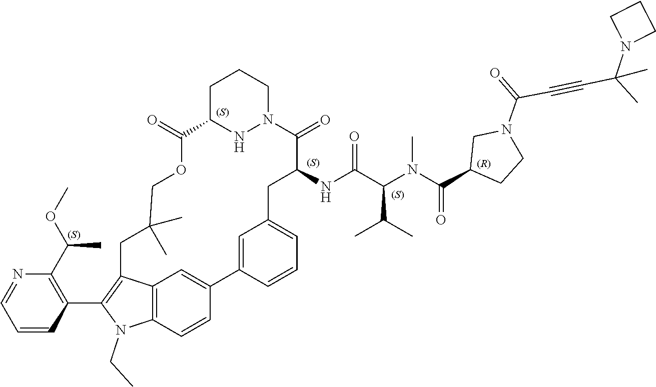 |
| BA477 | 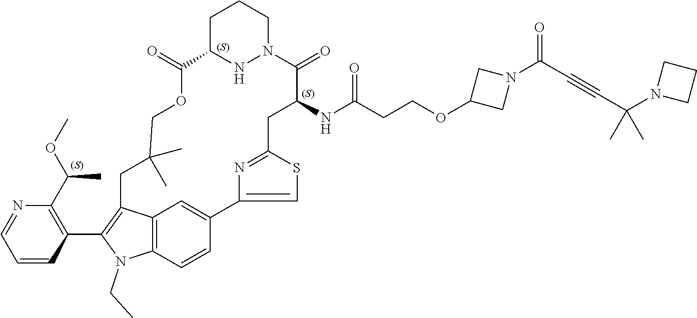 |
| BA478 | 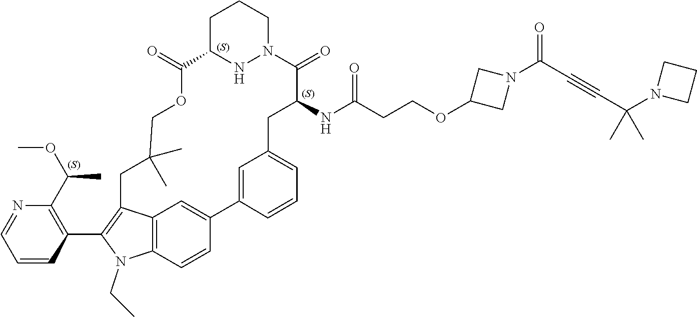 |
| BA479 | 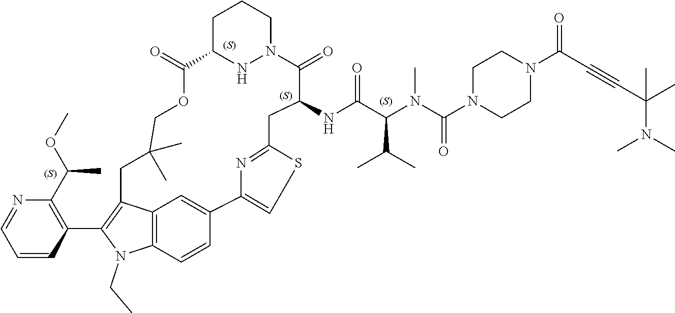 |
| BA480 | 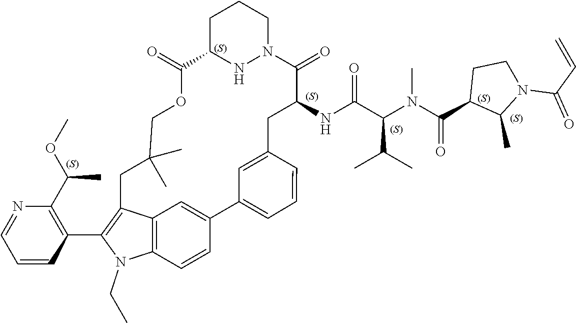 |
| BA481 | 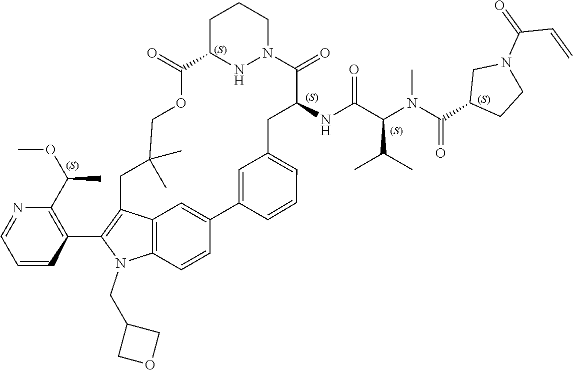 |
| BA482 | 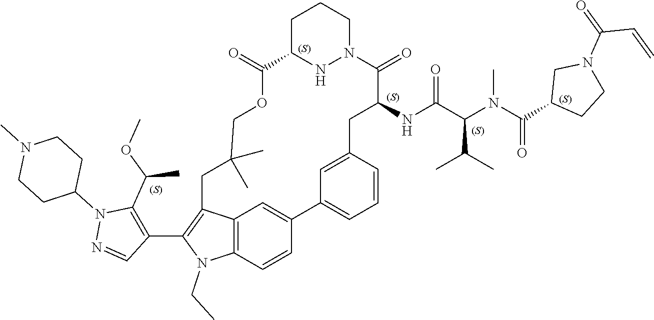 |
| BA483 | 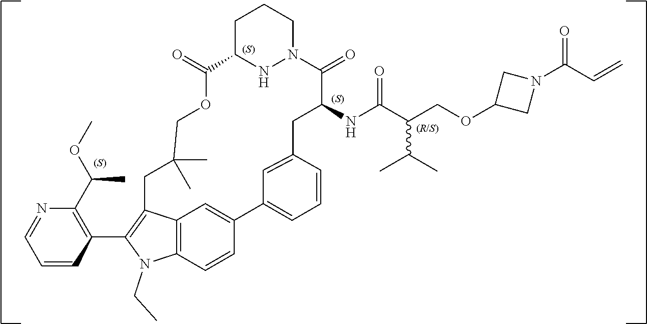 |
| BA484 | 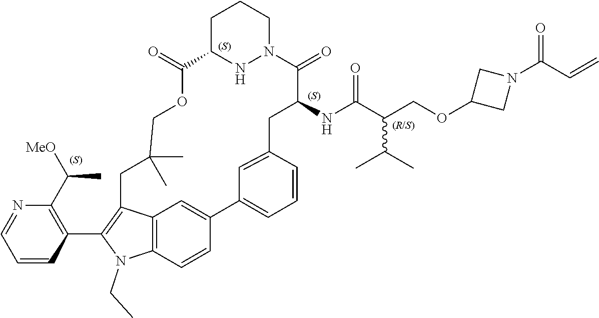 |
| BA485 | 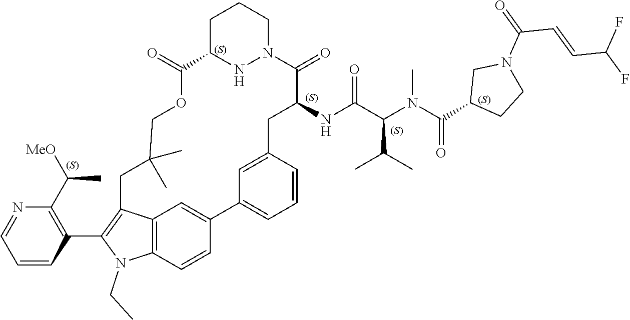 |
| BA486 | 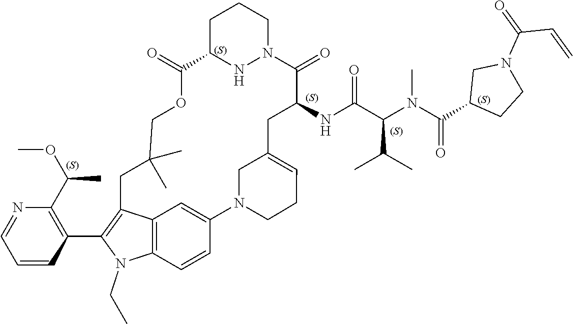 |
| BA487 | 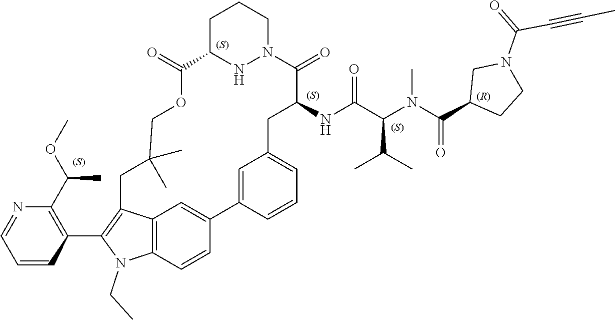 |
| BA488 | 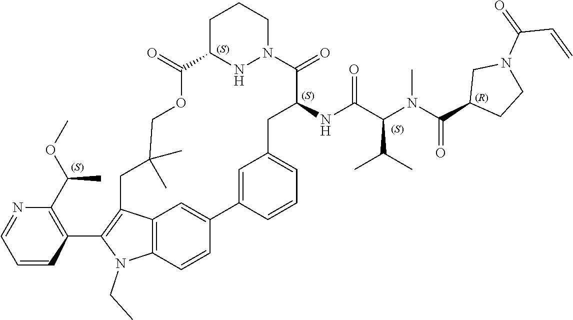 |
| BA489 | 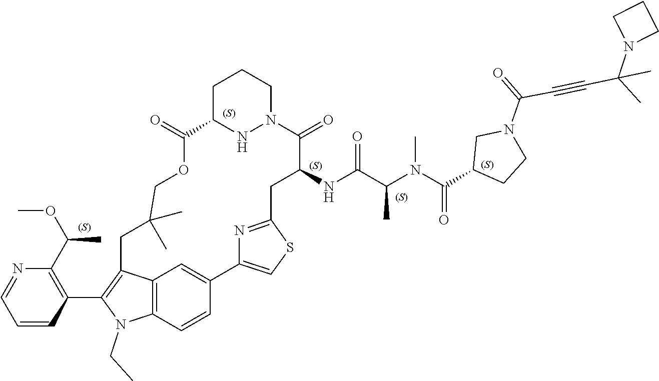 |
| BA490 | 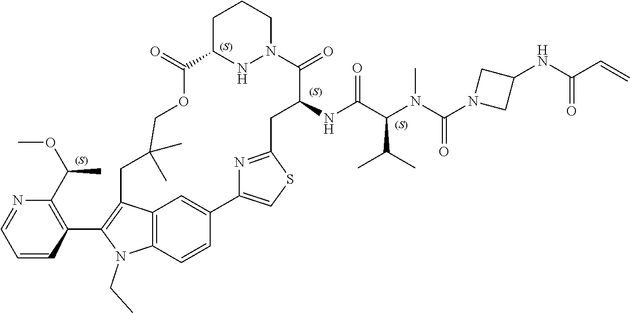 |
| BA491 | 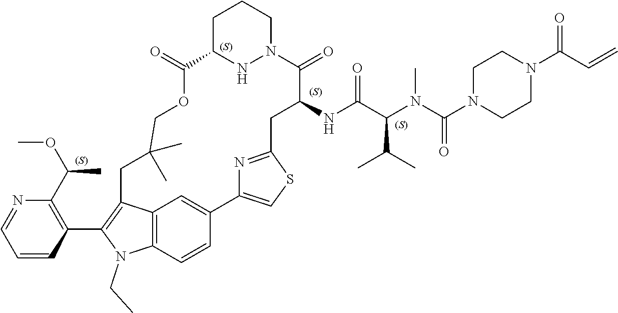 |
| BA492 | 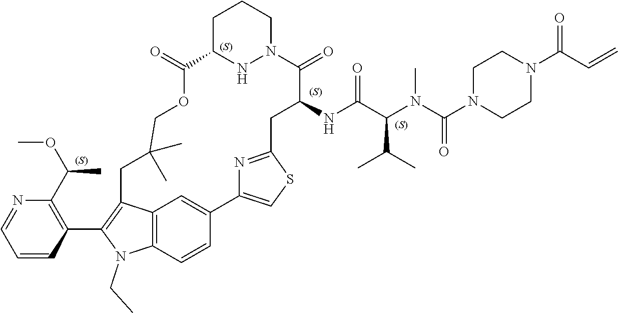 |
| BA493 | 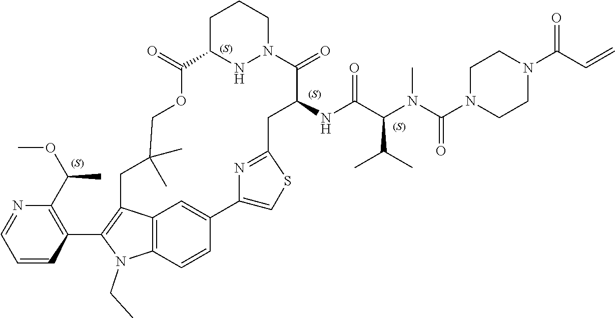 |
| BA494 | 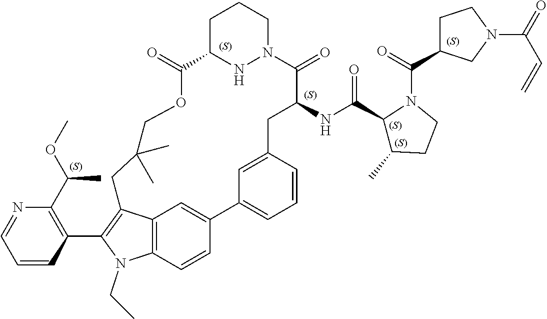 |
| BA495 | 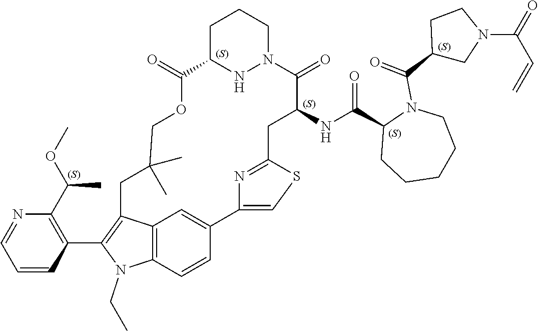 |
| BA496 | 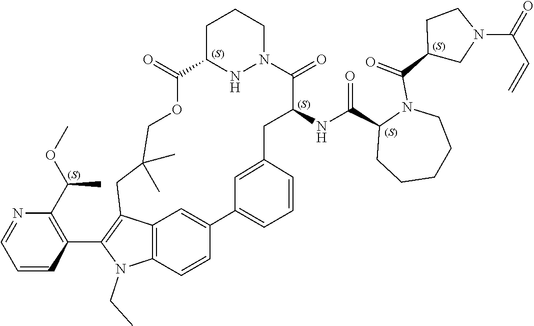 |
| BA497 | 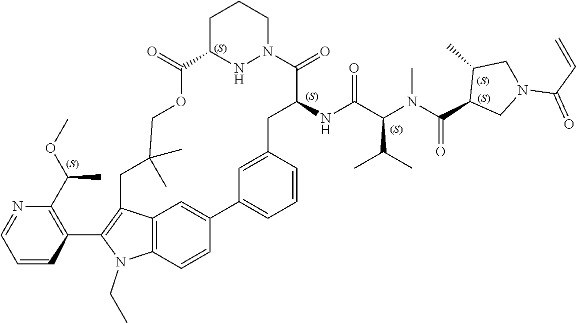 |
| BA498 | 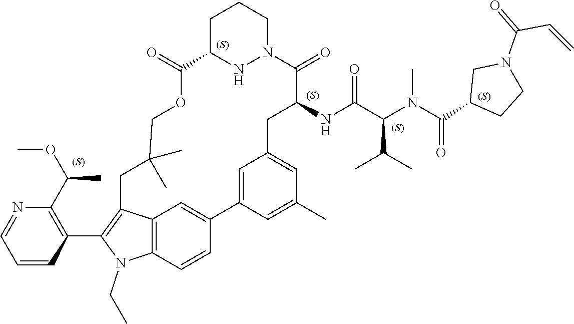 |
| BA499 | 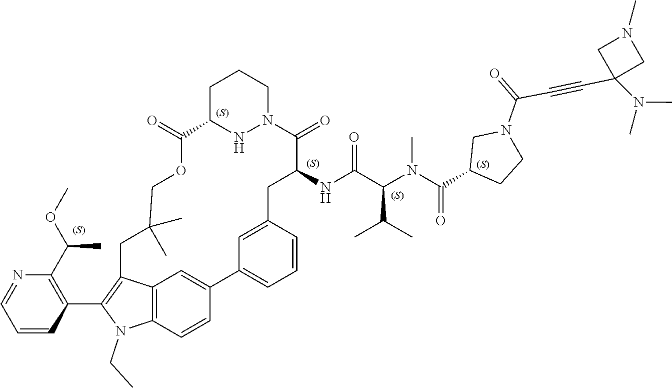 |
| BA500 | 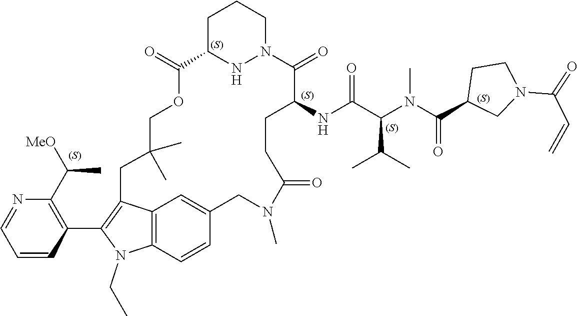 |
| BA501 | 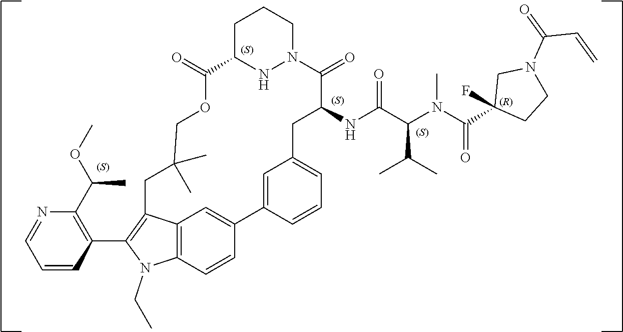 |
| BA502 | 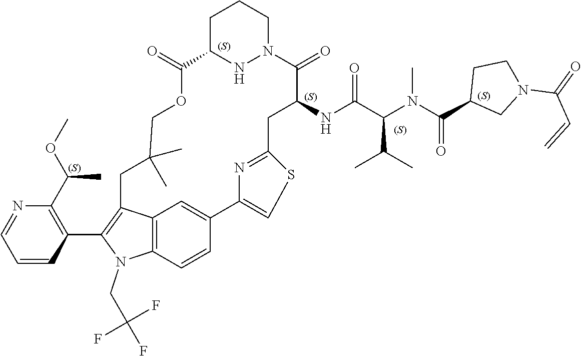 |
| BA503 | 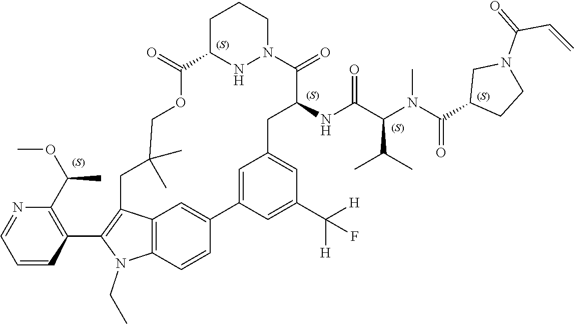 |
| BA504 | 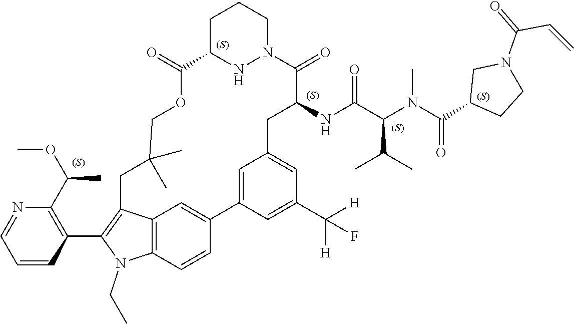 |
| BA505 | 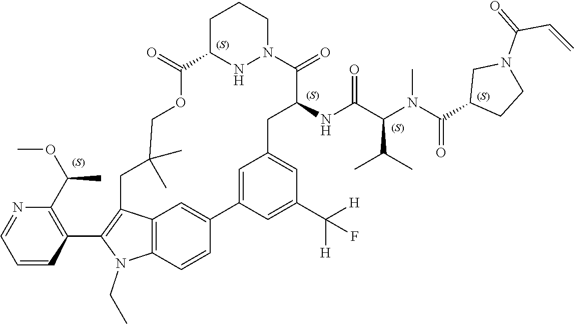 |
| BA506 | 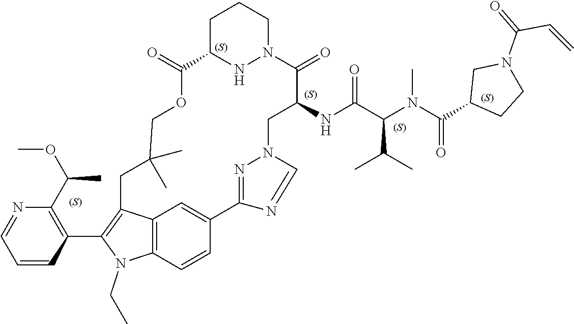 |
| BA507 | 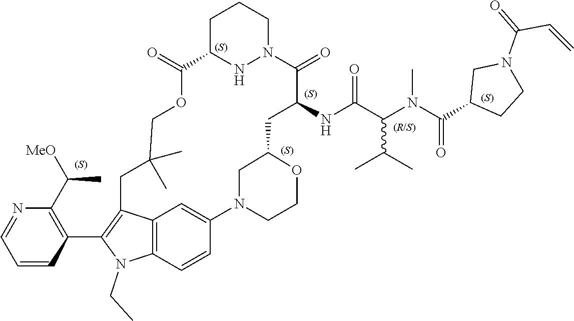 |
| BA508 | 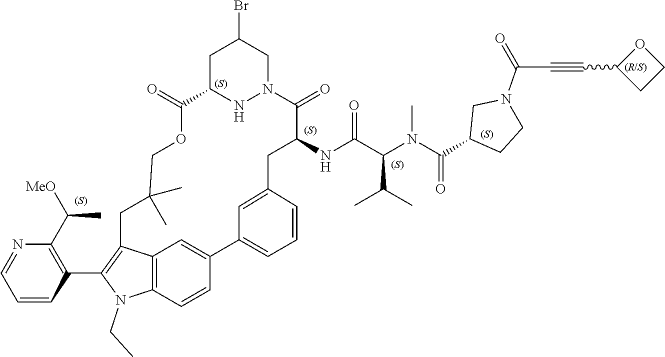 |
| BA509 | 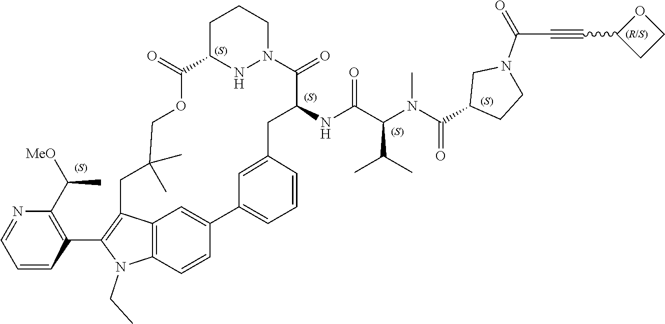 |
| BA510 | 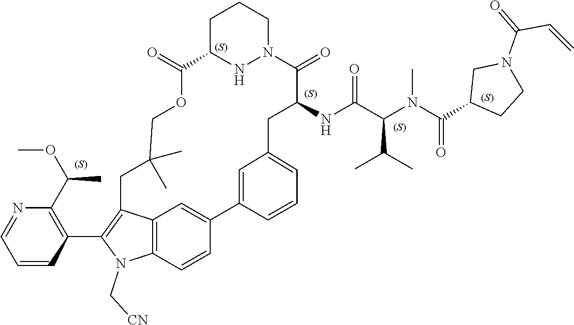 |
| BA511 | 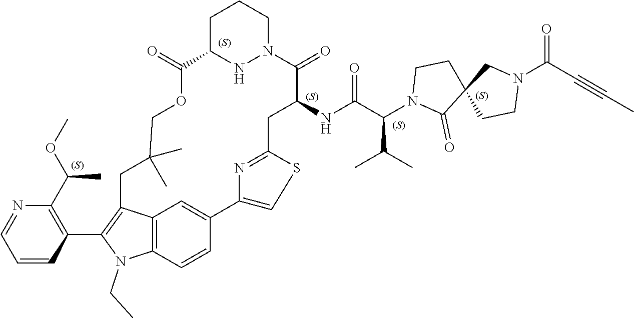 |
| BA512 | 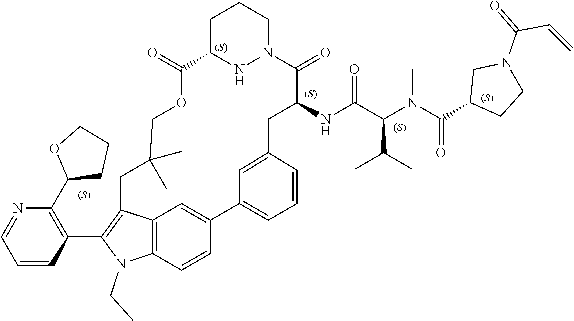 |
| BA513 | 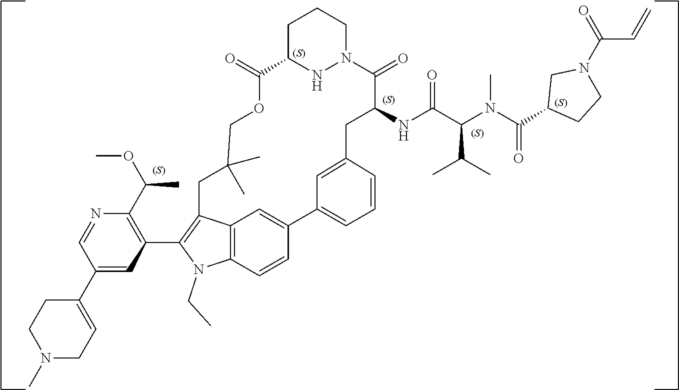 |
| BA514 | 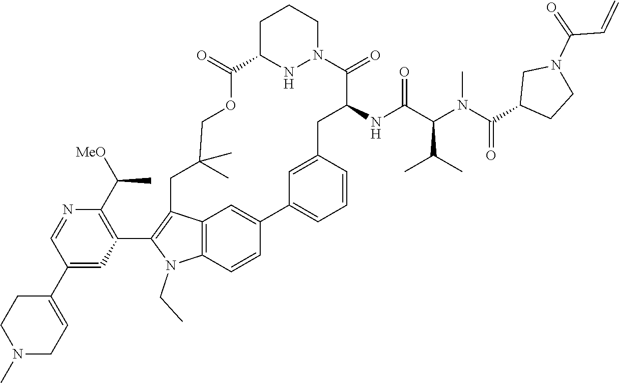 |
| BA515 | 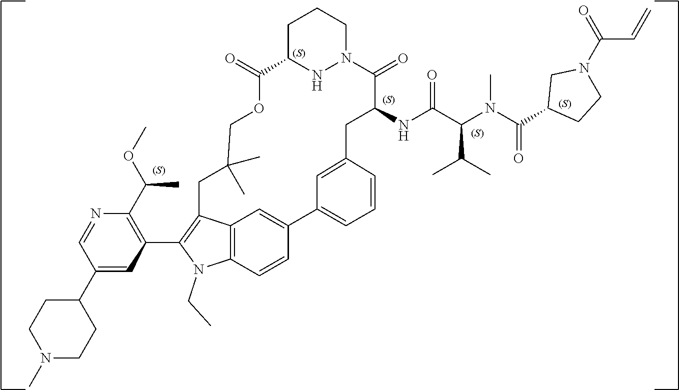 |
| BA516 | 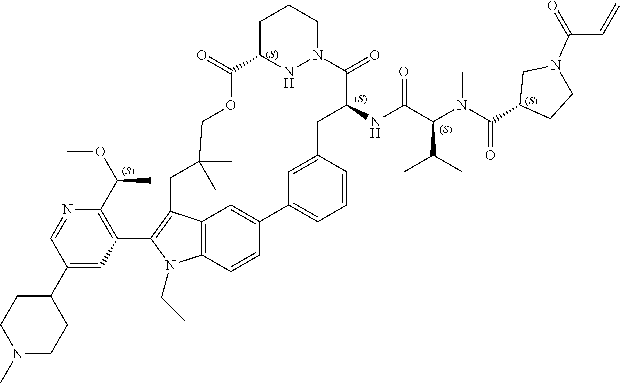 |
| BA517 | 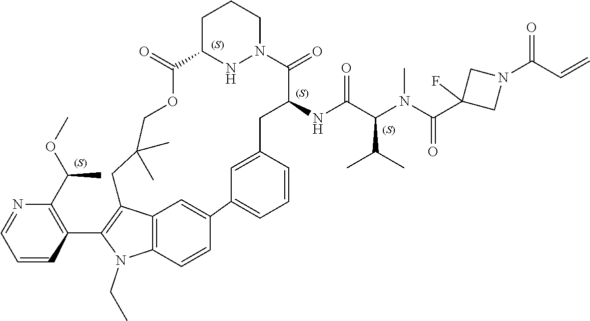 |
| BA518 | 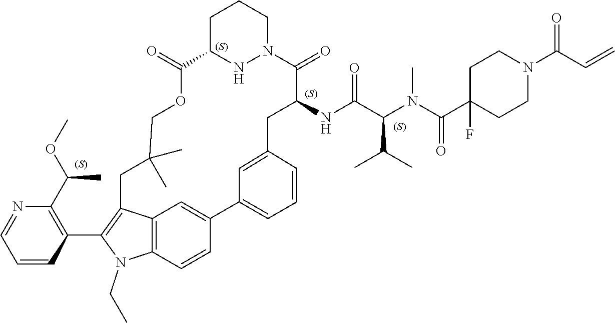 |
| BA519 | 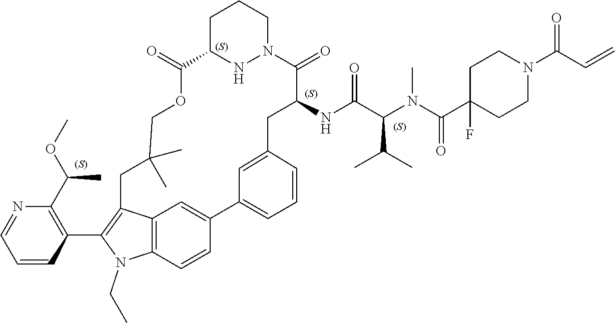 |
| BA520 | 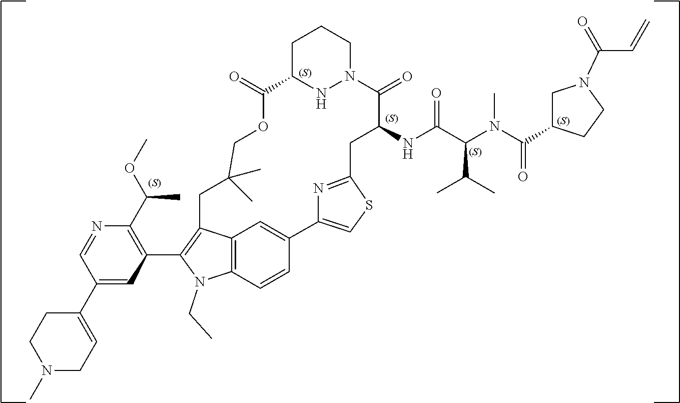 |
| BA521 | 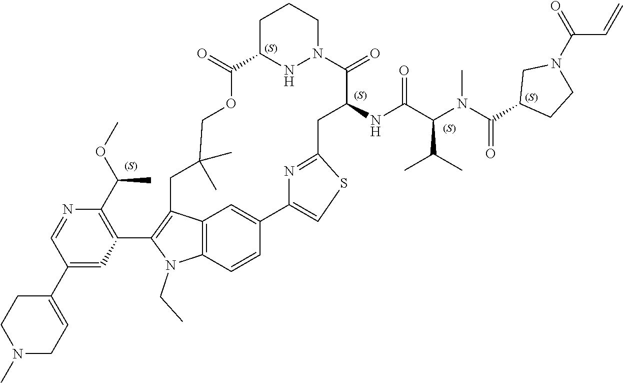 |
| BA522 | 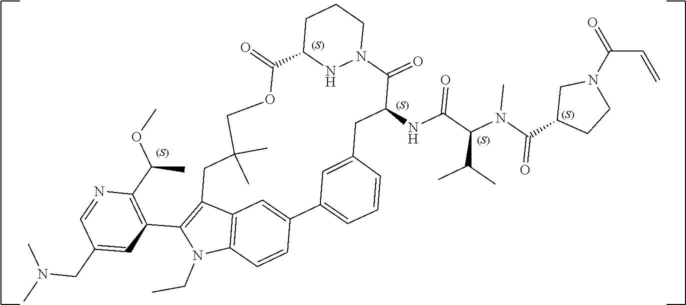 |
| BA523 | 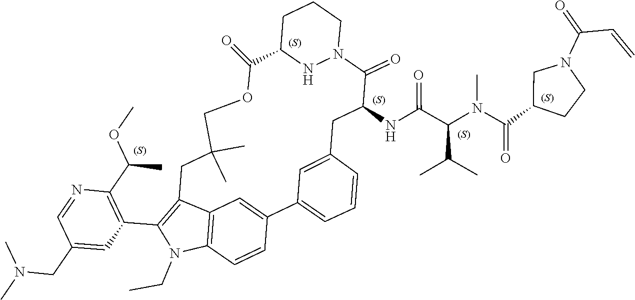 |
| BA524 | 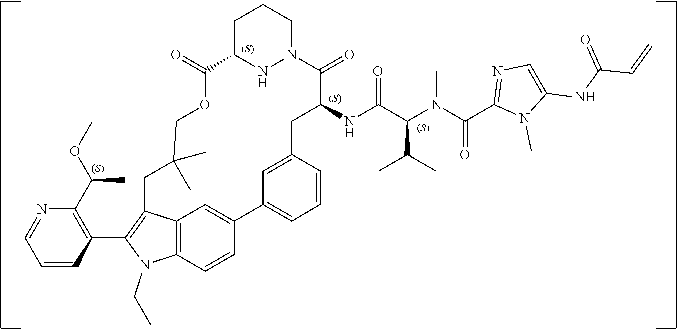 |
| BA525 | 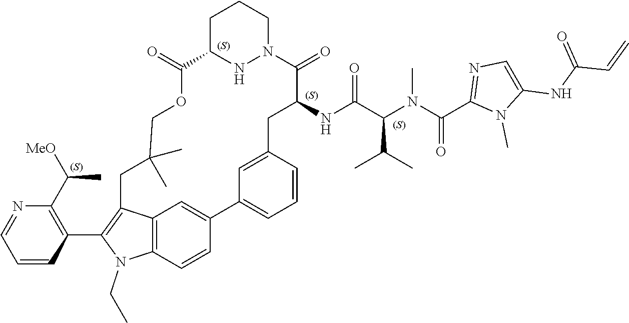 |
| BA526 | 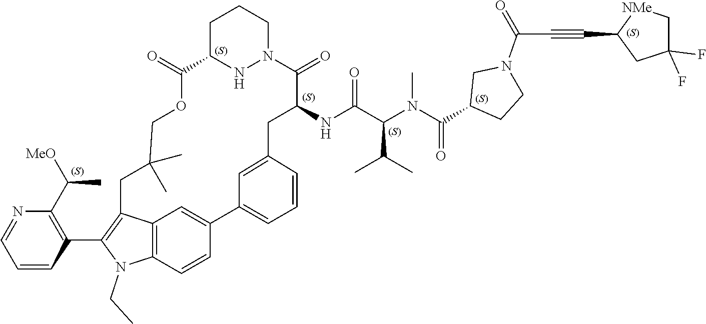 |
| BA527 | 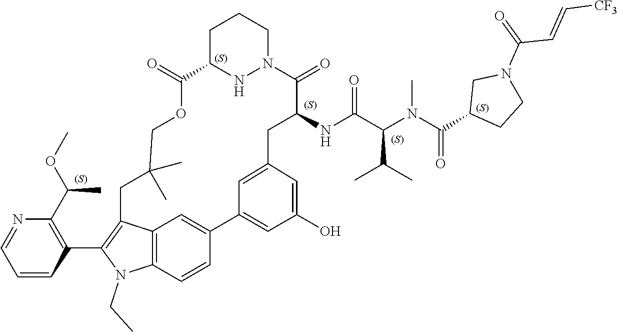 |
| BA528 | 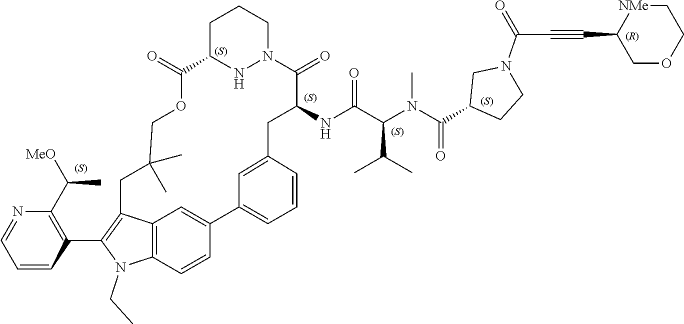 |
| BA529 | 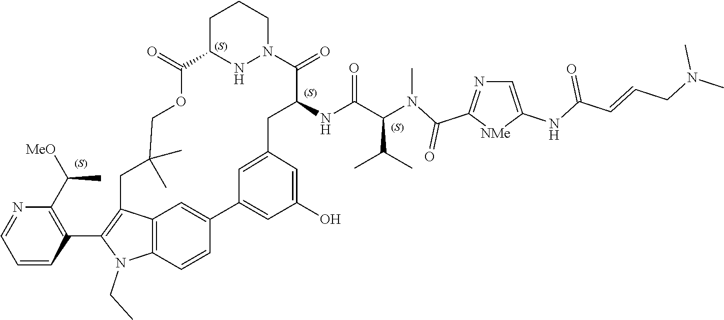 |
| BA530 | 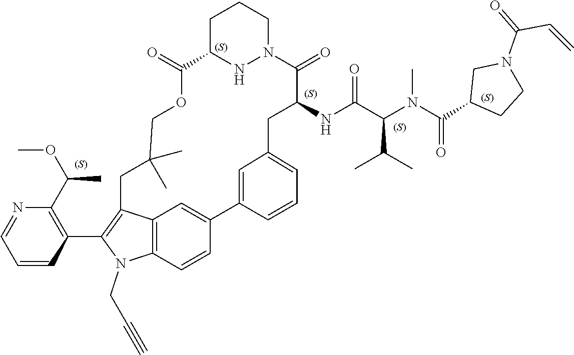 |
| BA531 | 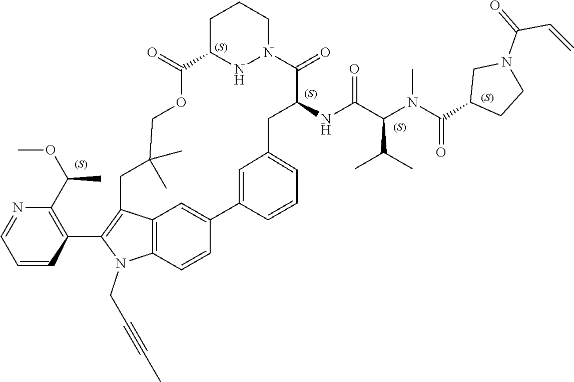 |
| BA532 | 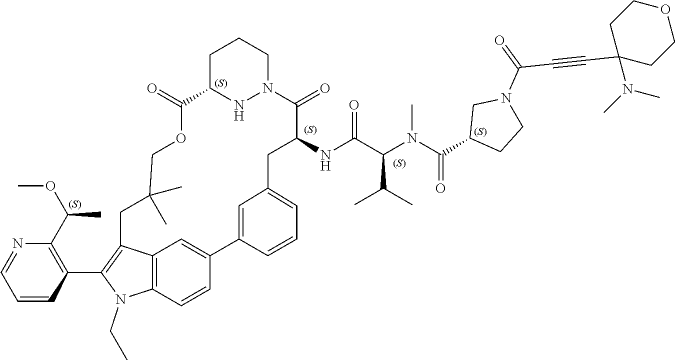 |
| BA533 | 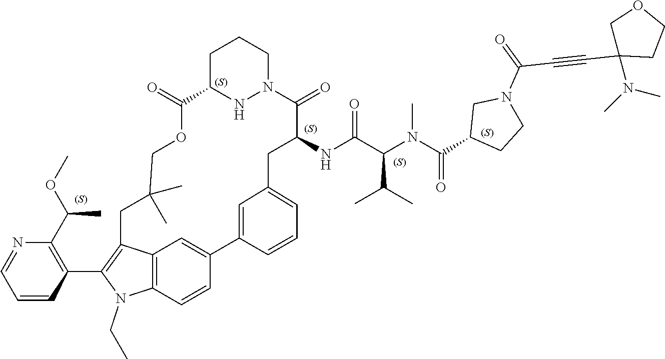 |
| BA534 | 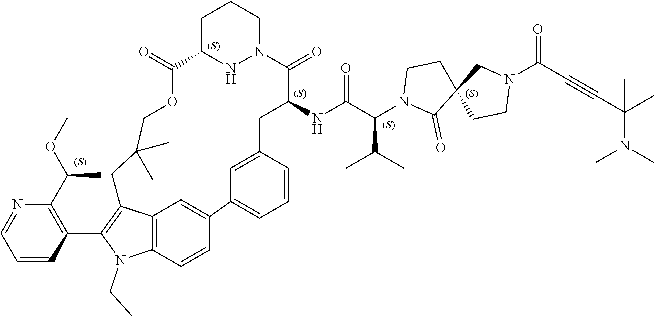 |
| BA535 | 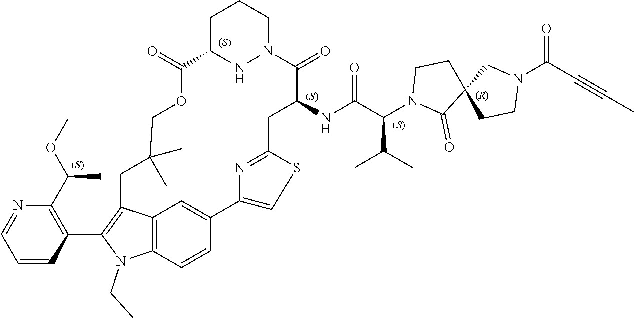 |
| BA536 | 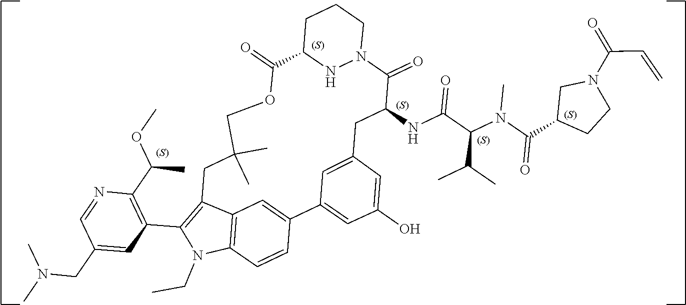 |
| BA537 | 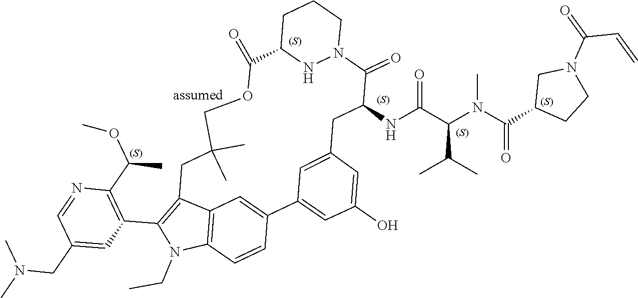 |
| BA538 | 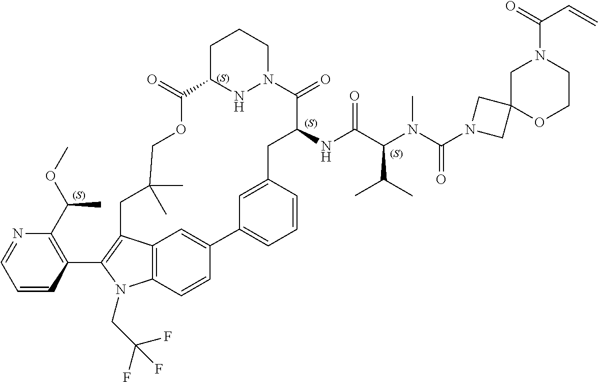 |
| BA539 | 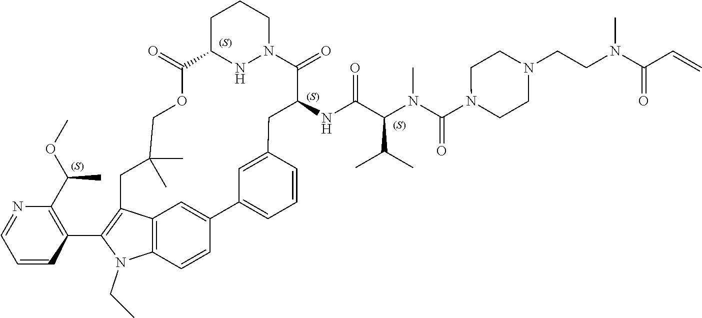 |
| BA540 | 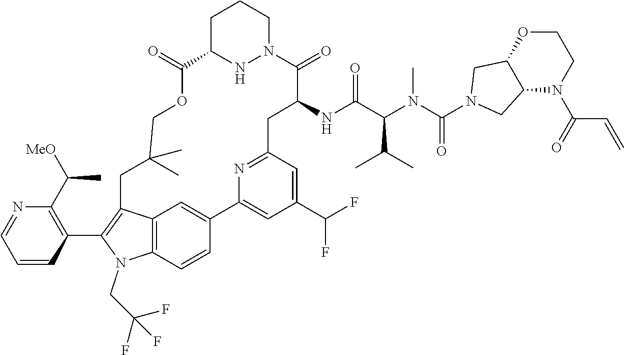 |
| BA541 | 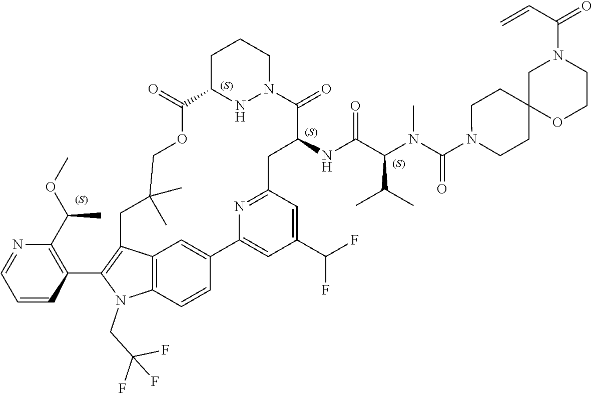 |
| BA542 | 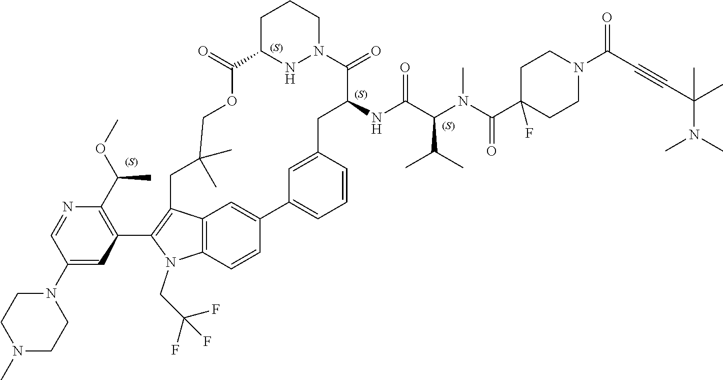 |
| BA543 | 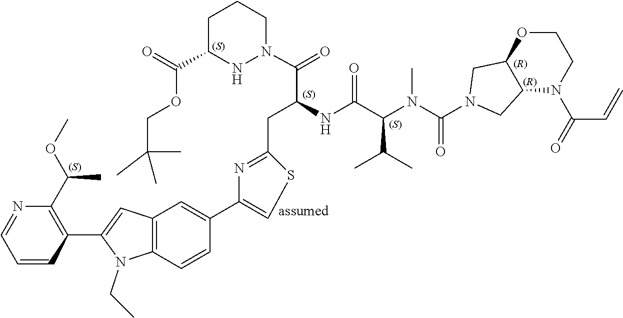 |
| BA544 | 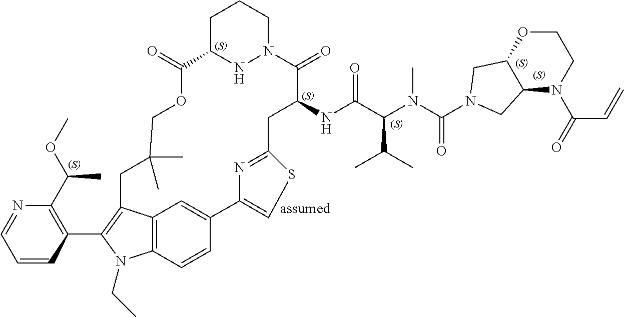 |
| BA545 | 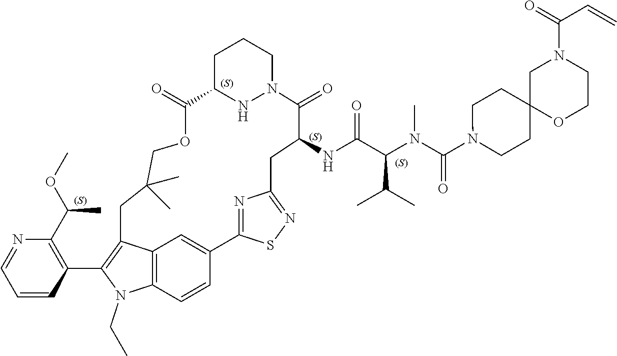 |
| BA546 | 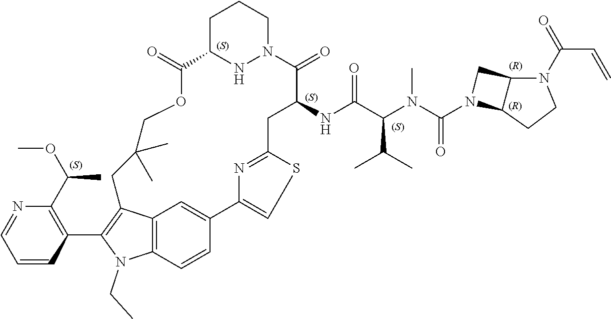 |
| BA547 | 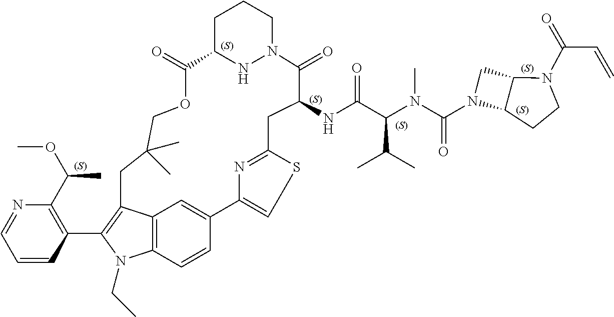 |
| BA548 | 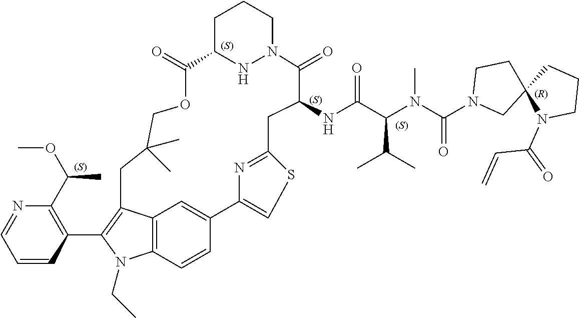 |
| BA549 | 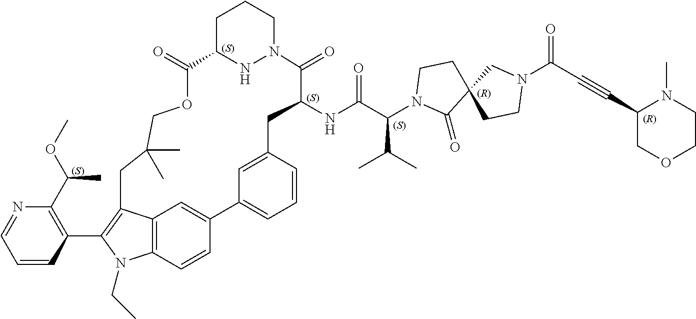 |
| BA550 | 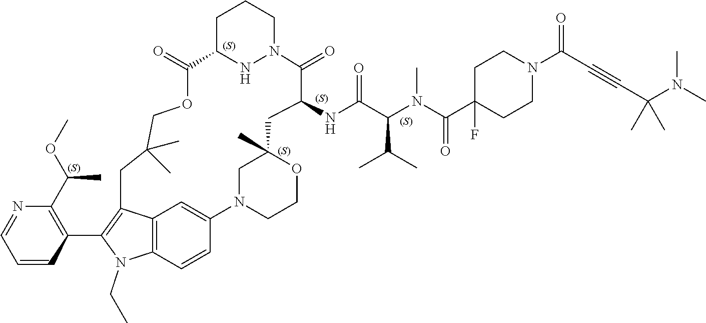 |
| BA551 | 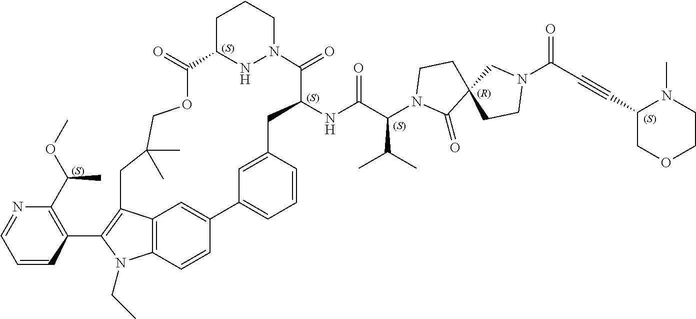 |
| BA552 | 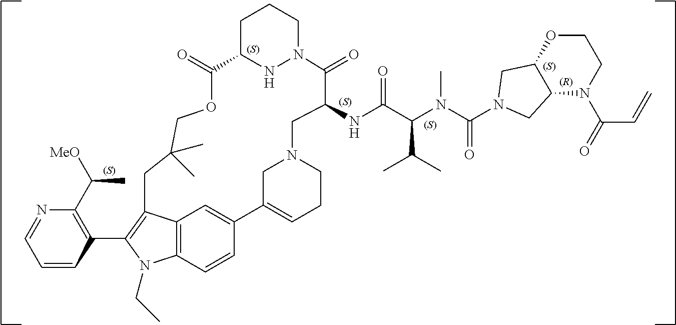 |
| BA553 | 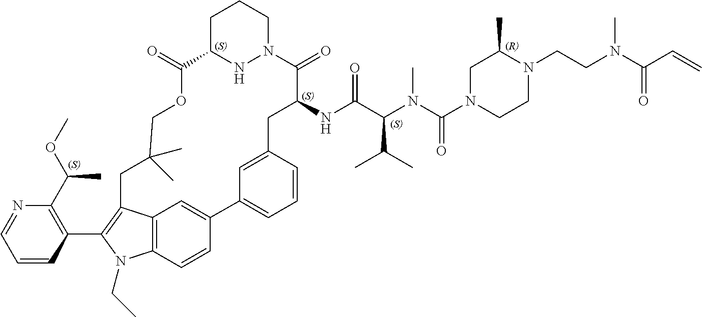 |
| BA554 | 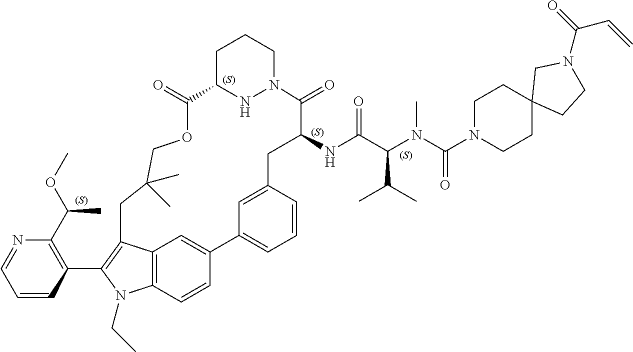 |
| BA555 | 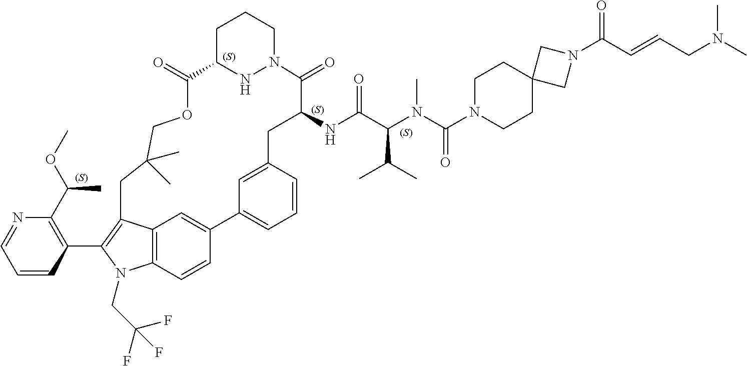 |
| BA556 | 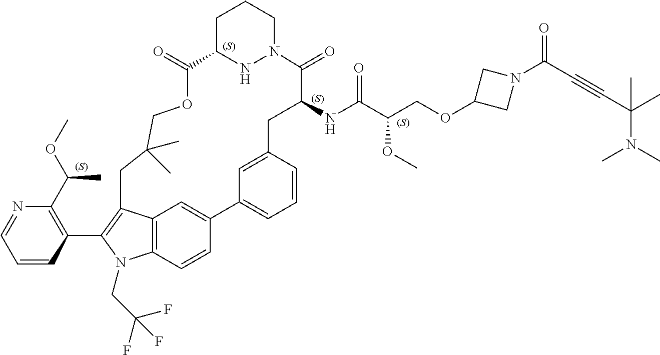 |
| BA557 | 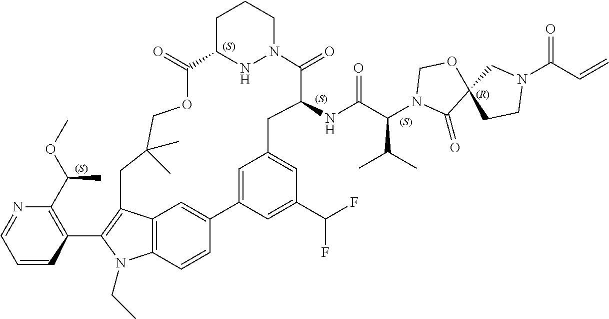 |
| BA558 | 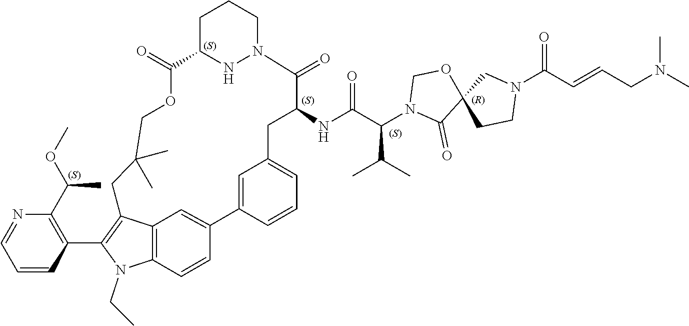 |
| BA559 | 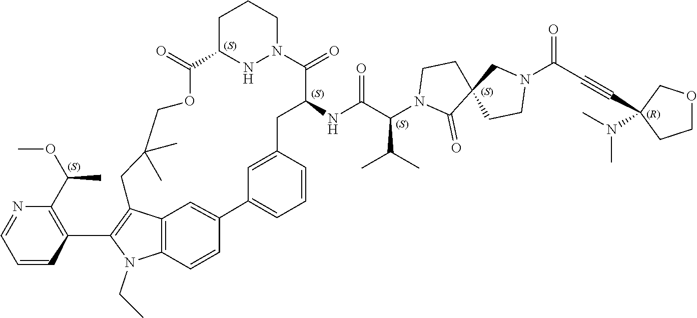 |
| BA560 | 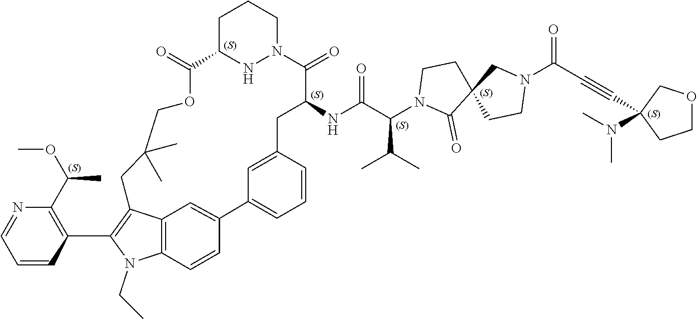 |
| BA561 | 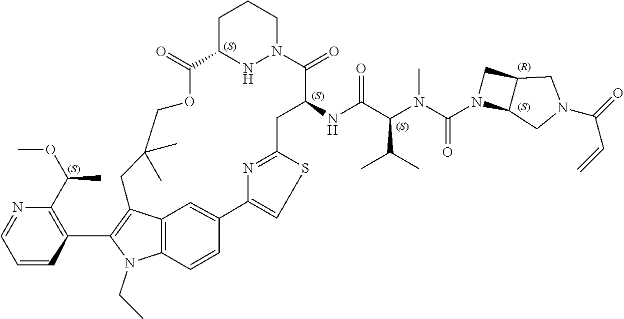 |
| BA562 | 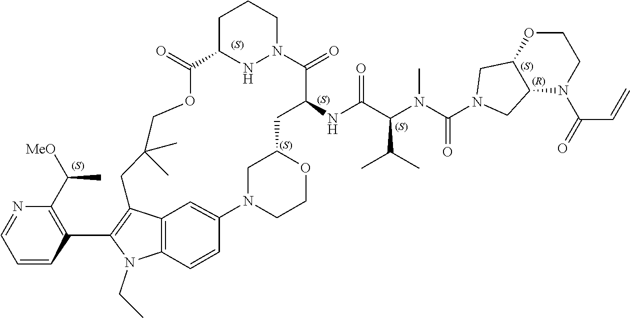 |
| BA563 | 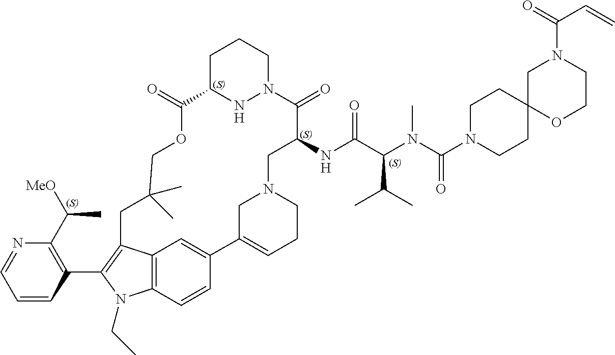 |
| BA564 | 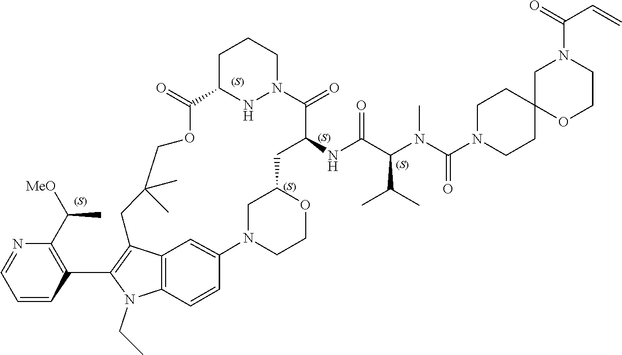 |
| BA565 | 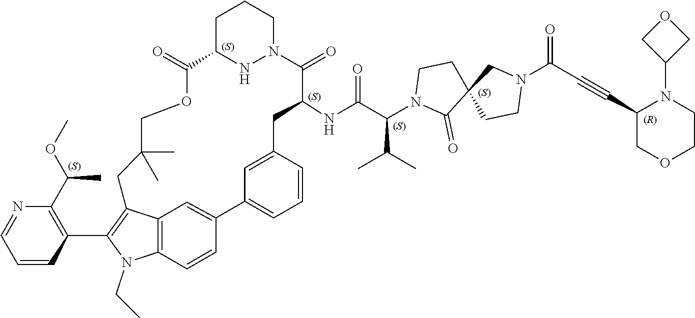 |
| BA566 | 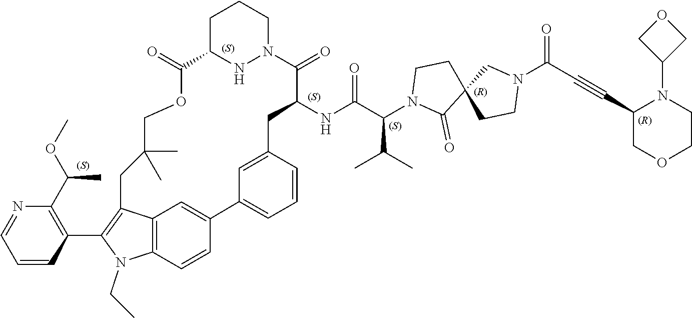 |
| BA567 | 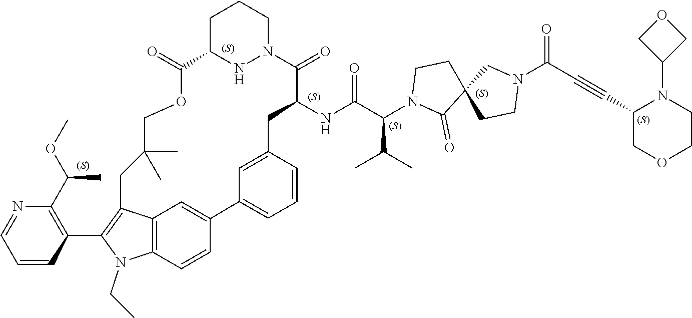 |
| BA568 | 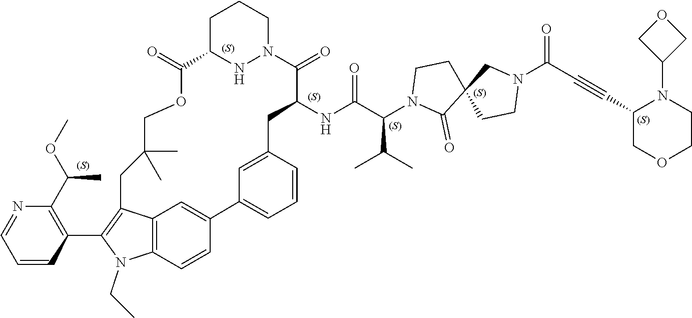 |
| BA569 | 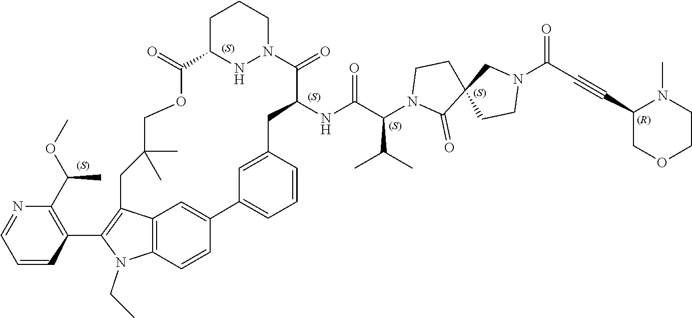 |
| BA570 | 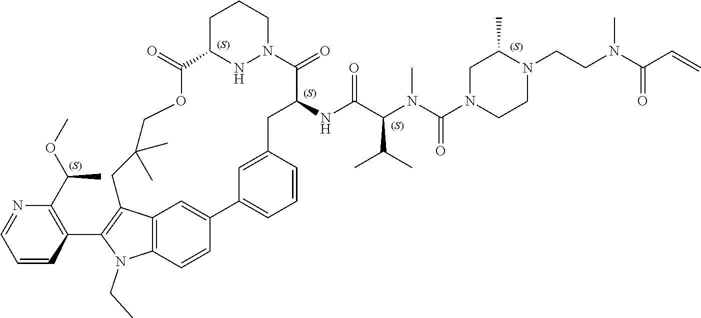 |
| BA571 | 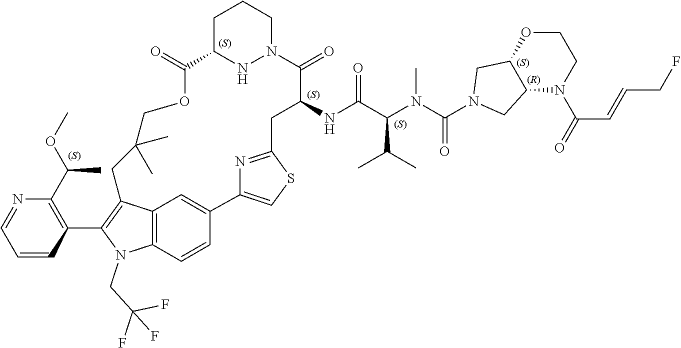 |
| BA572 | 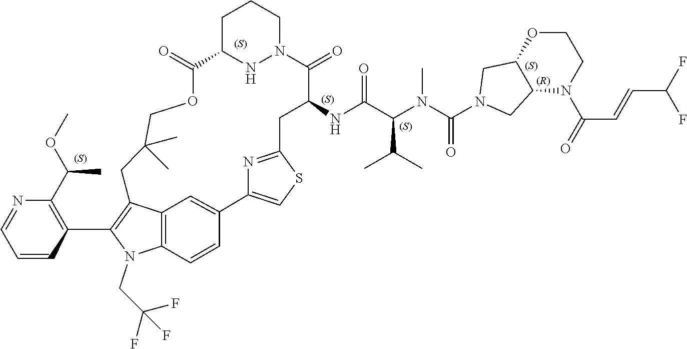 |
| BA573 | 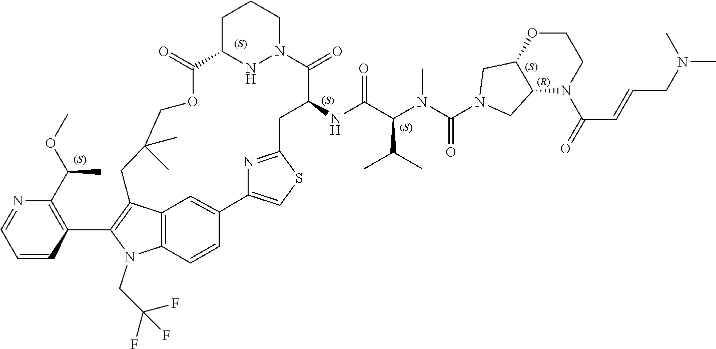 |
| BA574 | 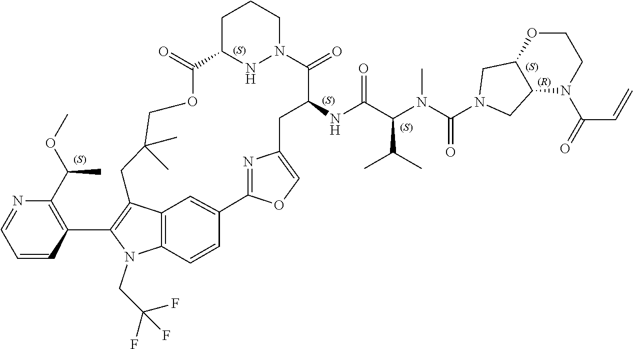 |
| BA575 | 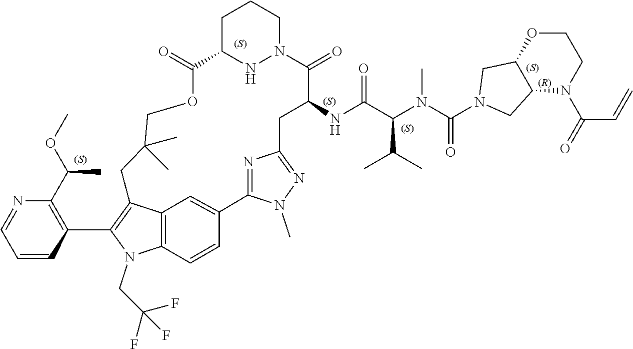 |
| BA576 | 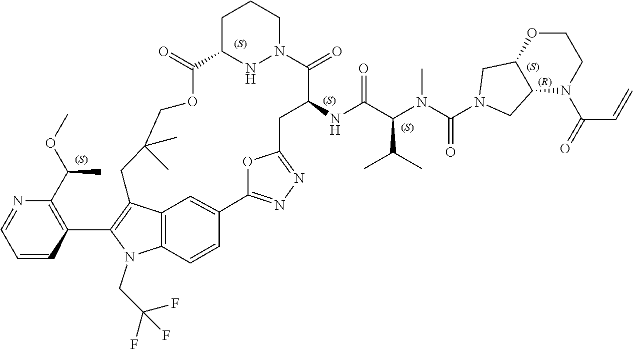 |
| BA577 | 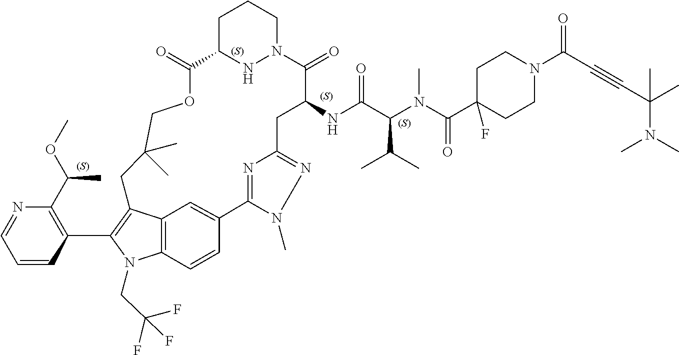 |
| BA578 | 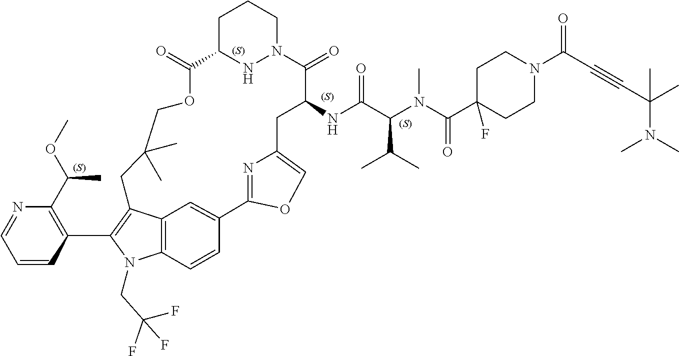 |
| BA579 | 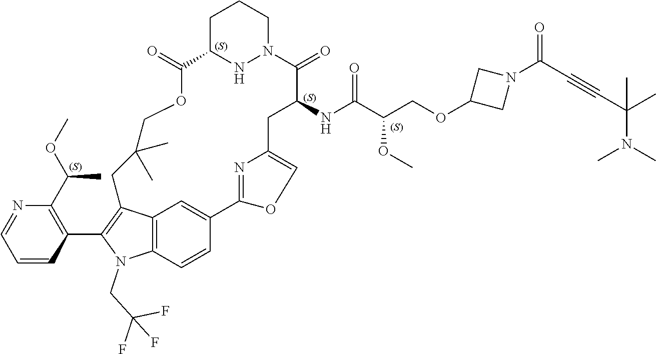 |
| BA580 | 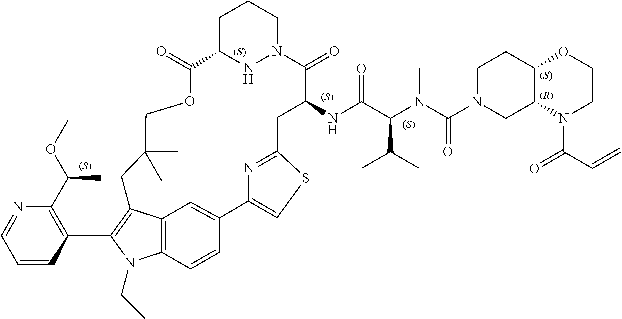 |
| BA581 | 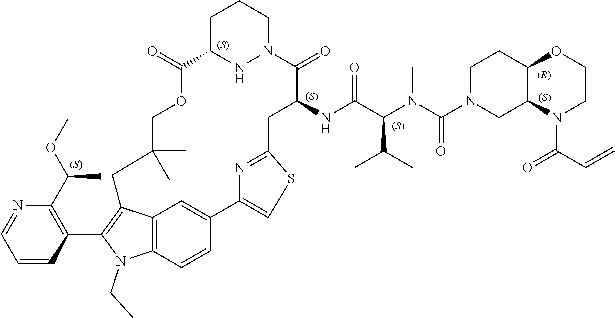 |
| BA582 | 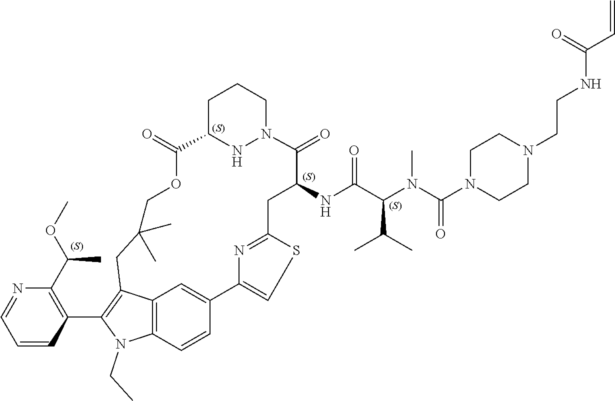 |
| BA583 | 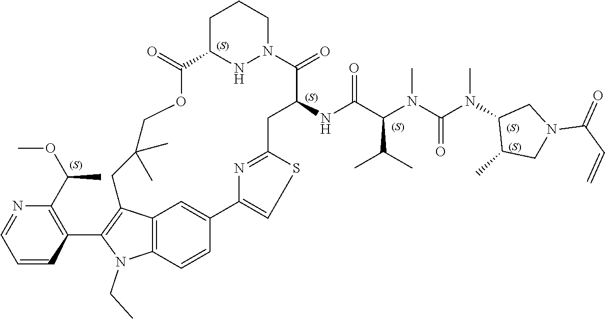 |
| BA584 | 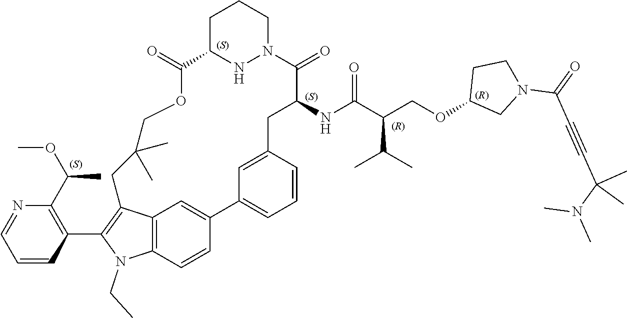 |
| BA585 | 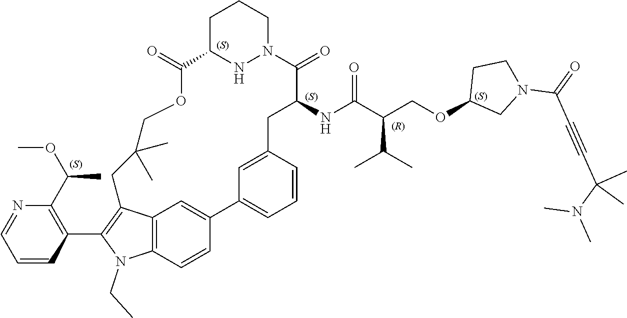 |
| BA586 | 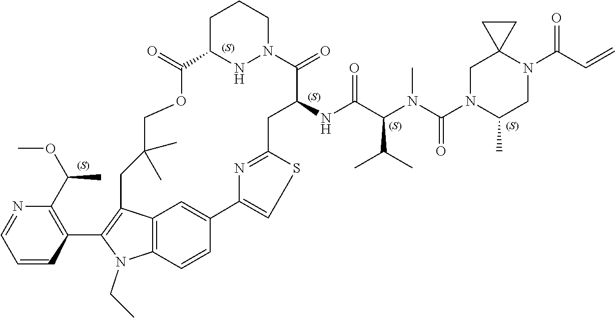 |
| BA587 | 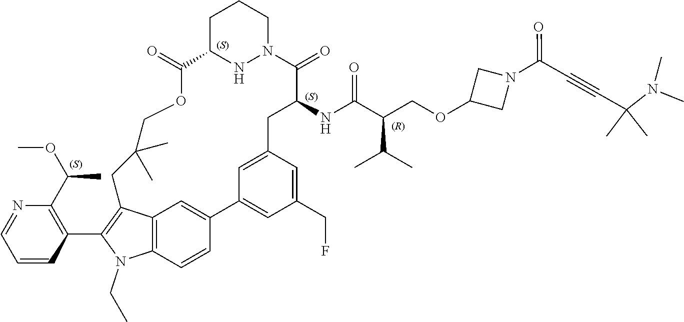 |
| BA588 | 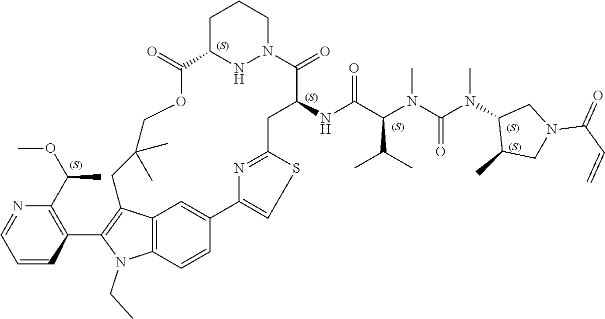 |
| BA589 | 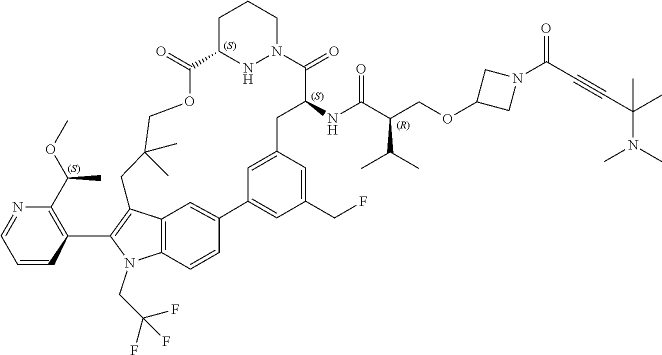 |
| BA590 | 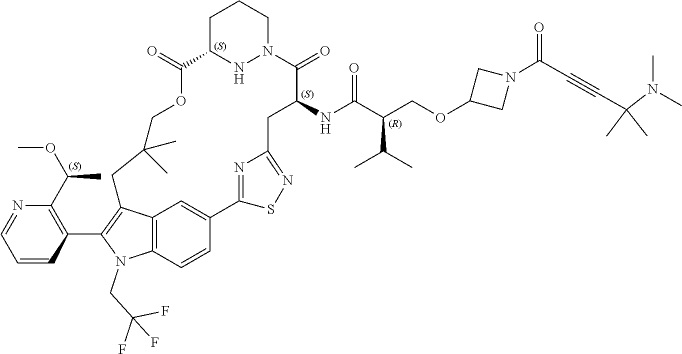 |
| BA591 | 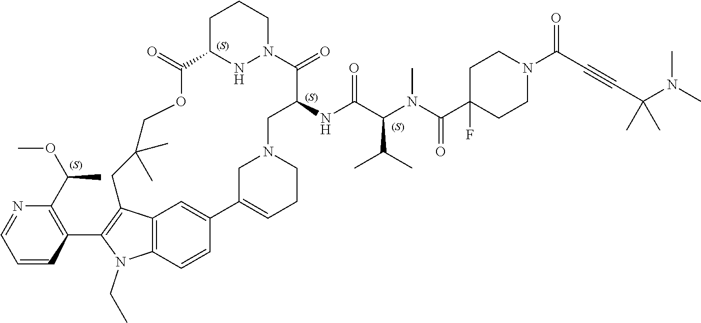 |
| BA592 | 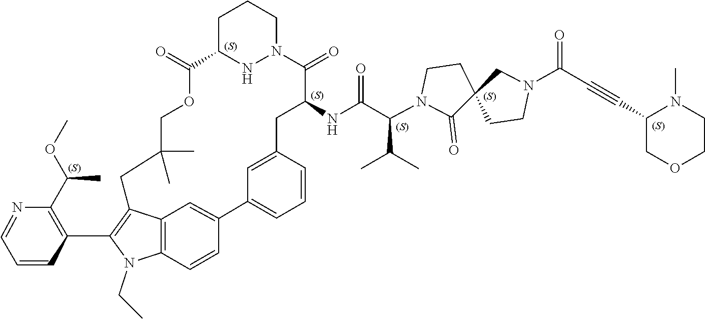 |
| BA593 | 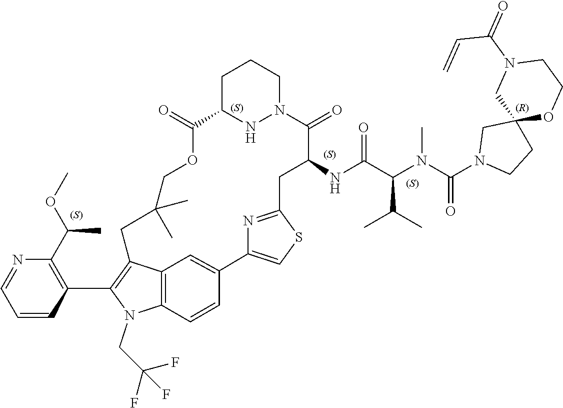 |
| BA594 | 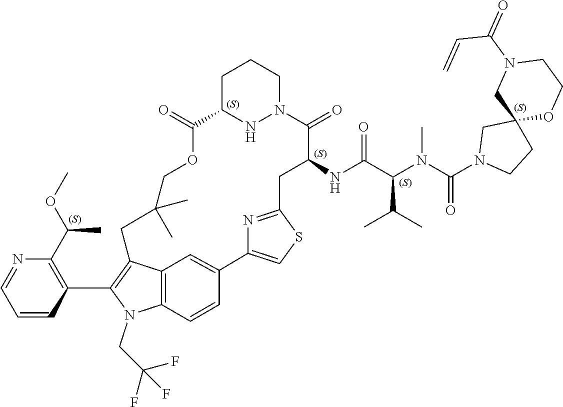 |
| BA595 | 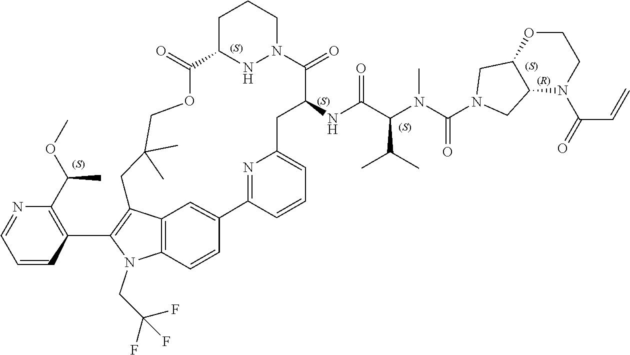 |
| BA596 | 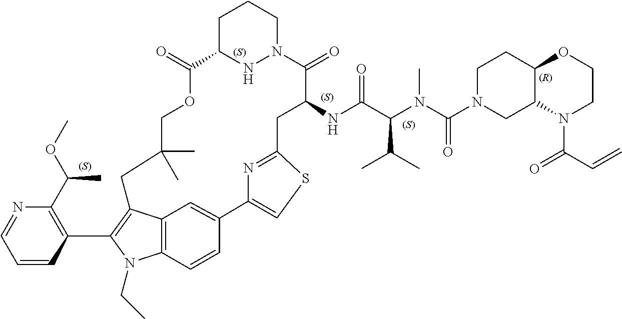 |
| BA597 | 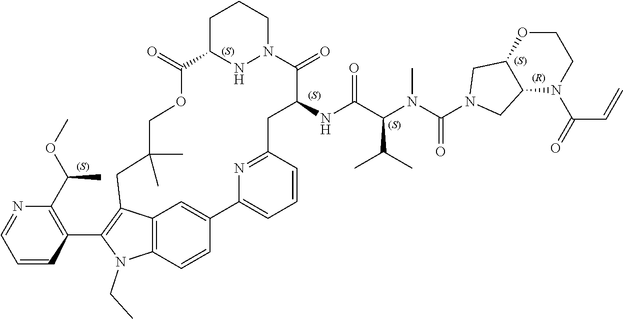 |
| BA598 | 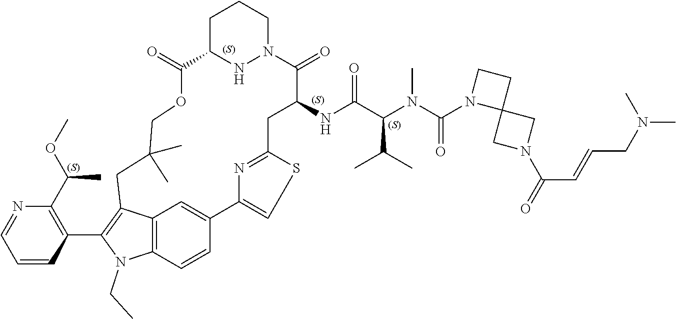 |
| BA599 | 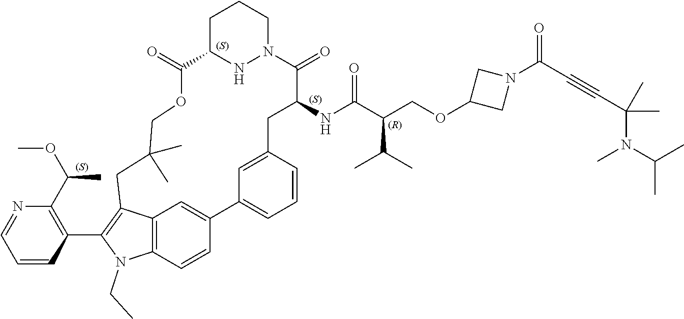 |
| BA600 | 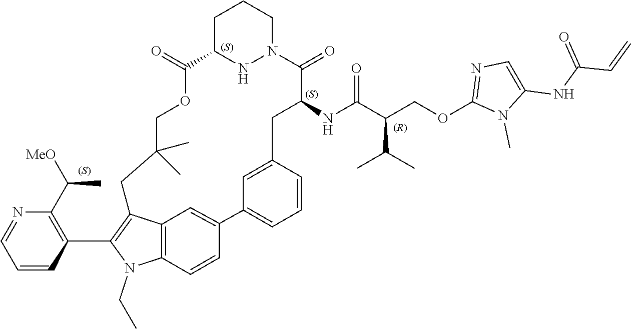 |
| BA601 | 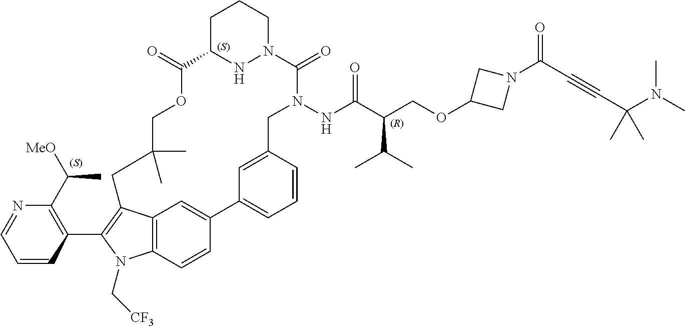 |
| BA602 | 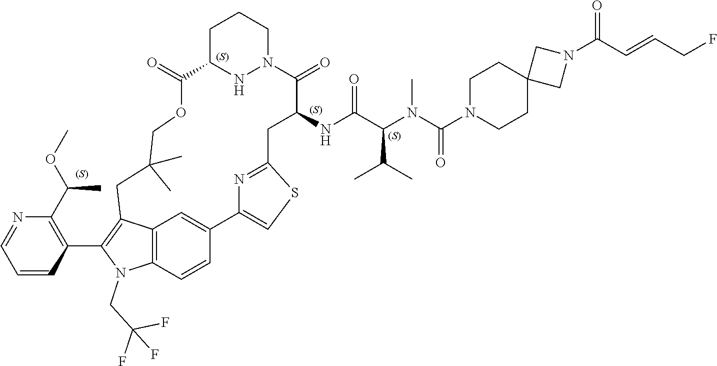 |
| BA603 | 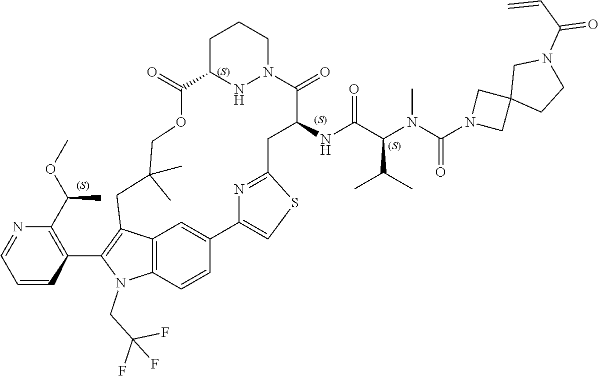 |
| BA604 | 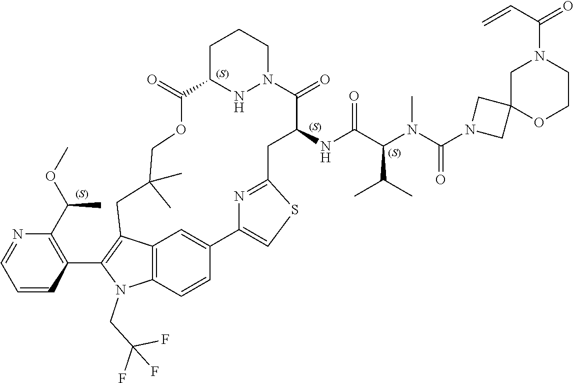 |
| BA605 | 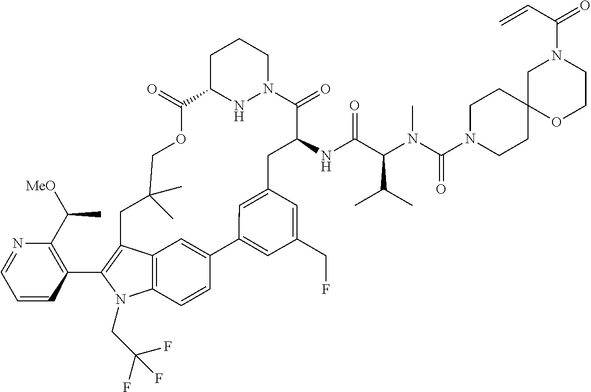 |
| BA606 | 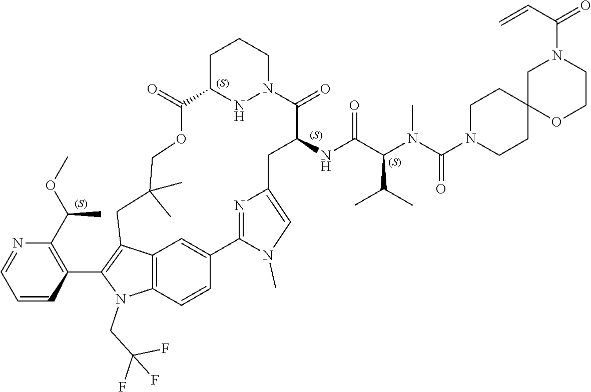 |
| BA607 | 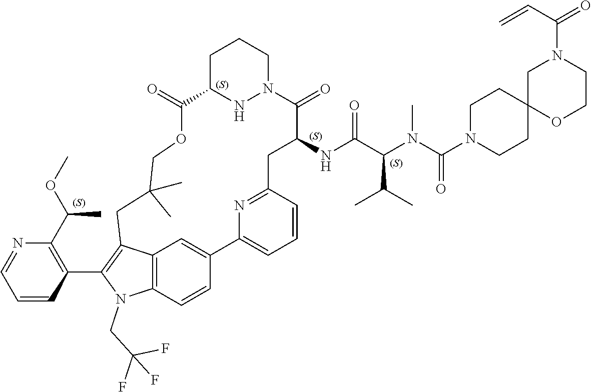 |
| BA608 | 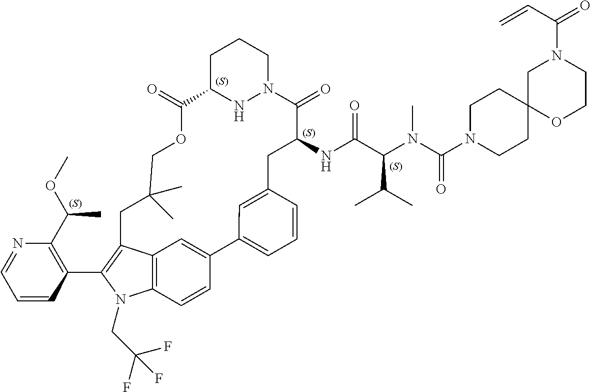 |
| BA609 | 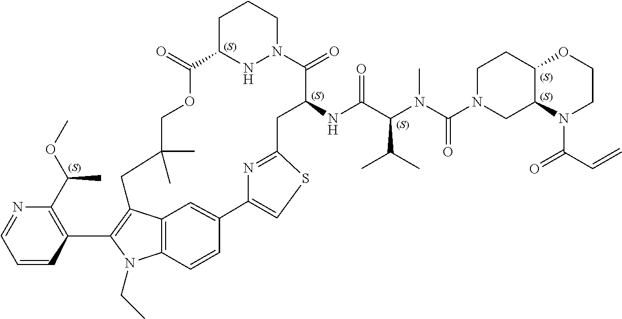 |
| BA610 | 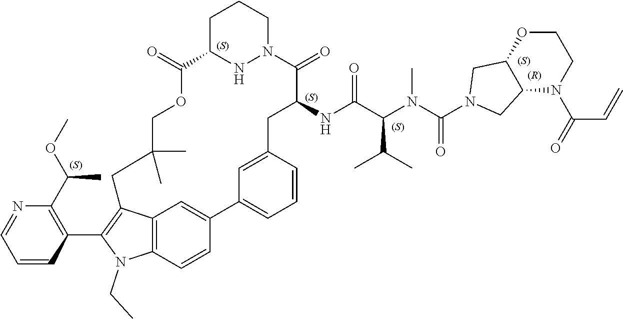 |
| BA611 | 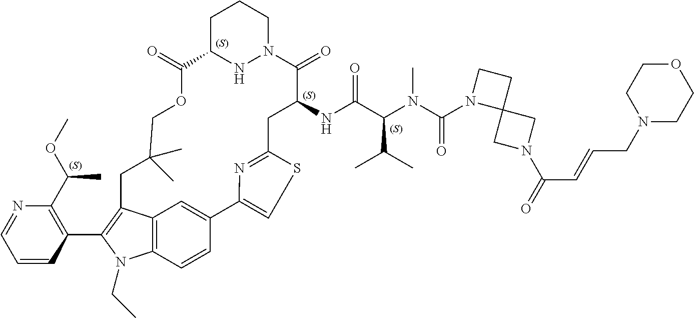 |
| BA612 | 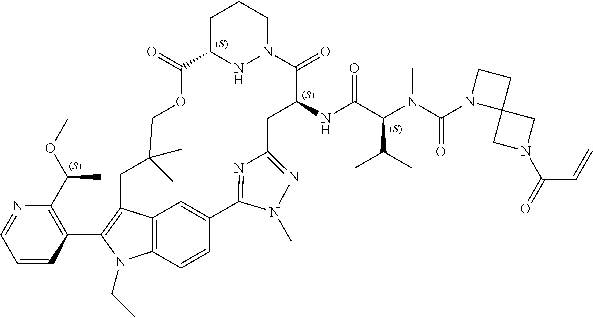 |
| BA613 | 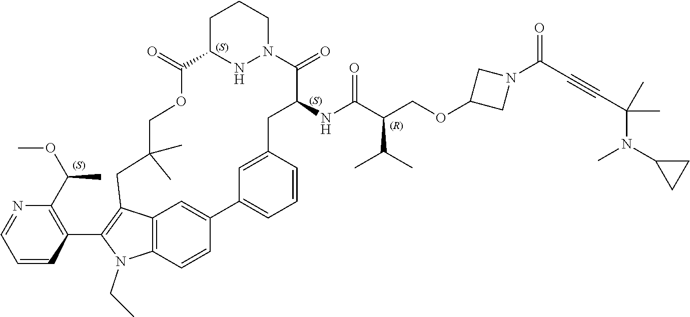 |
| BA614 | 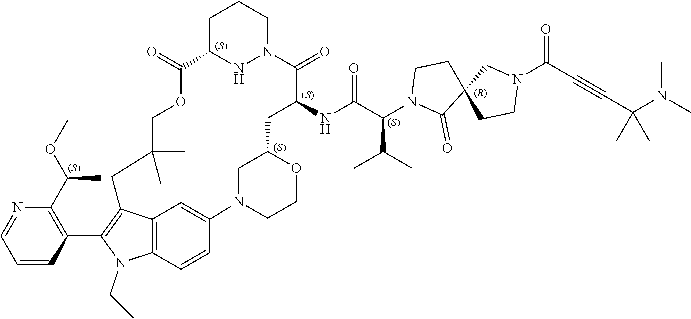 |
| BA615 | 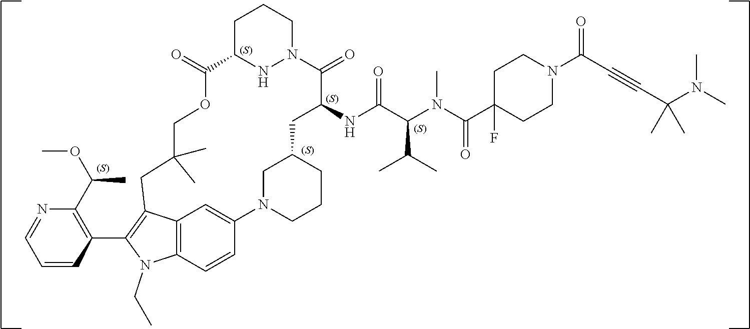 |
| BA616 | 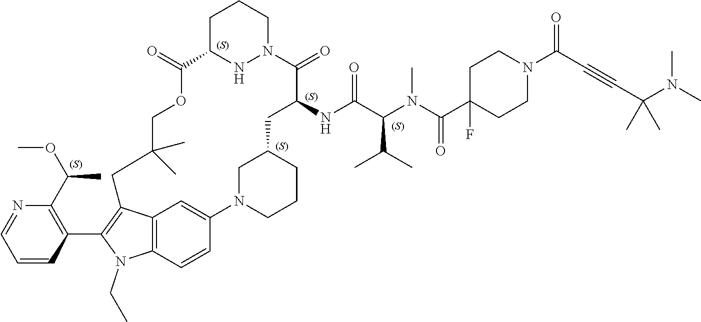 |
| BA617 | 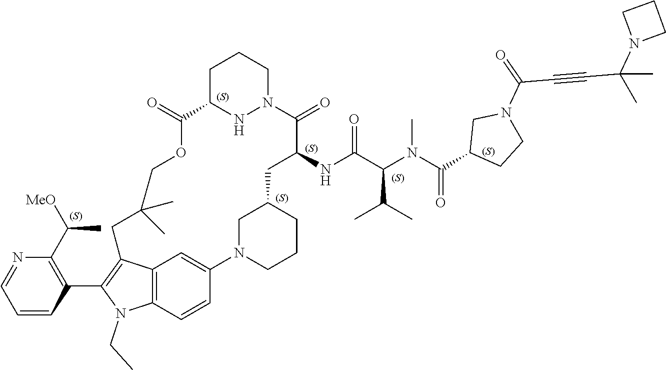 |
| BA618 | 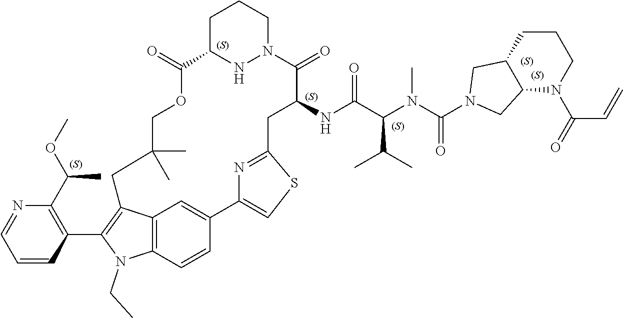 |
| BA619 | 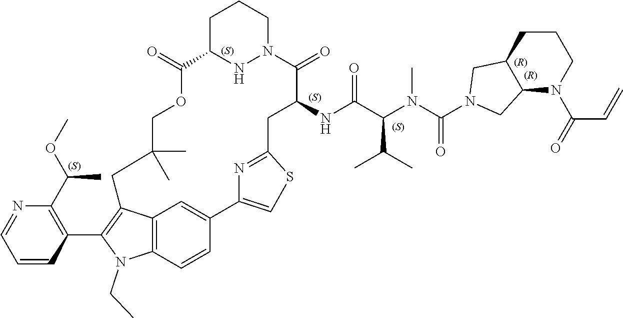 |
| BA620 | 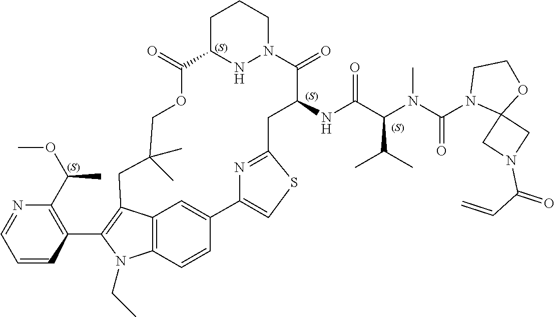 |
| BA621 | 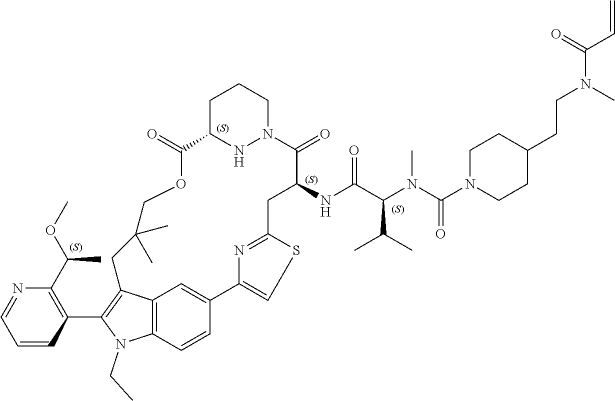 |
| BA622 | 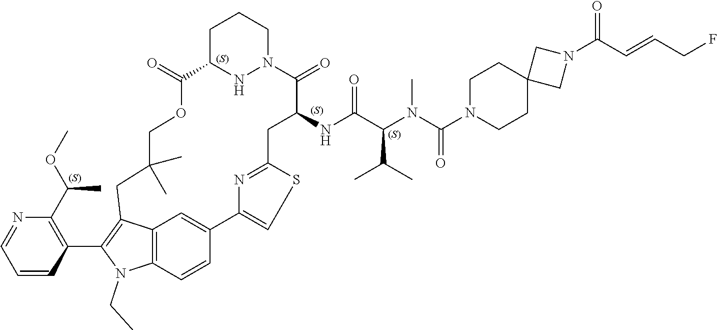 |
| BA623 | 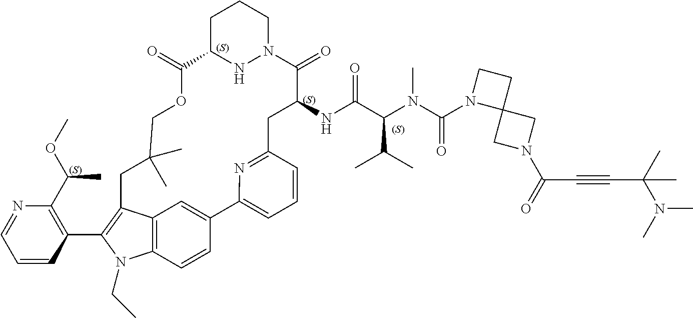 |
| BA624 | 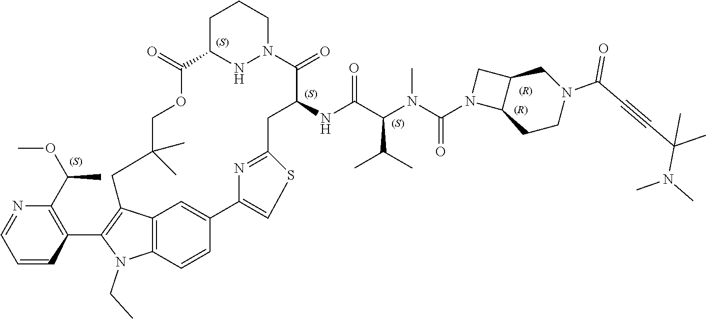 |
| BA625 | 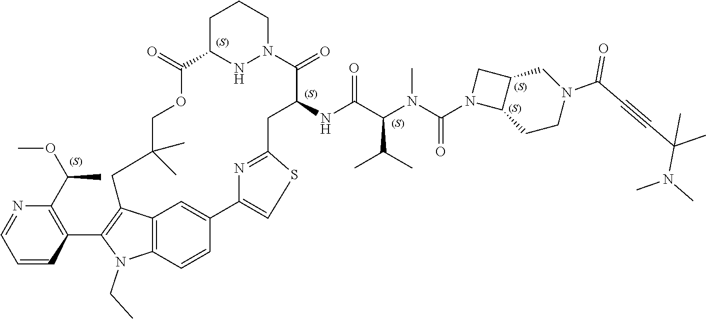 |
| BA626 | 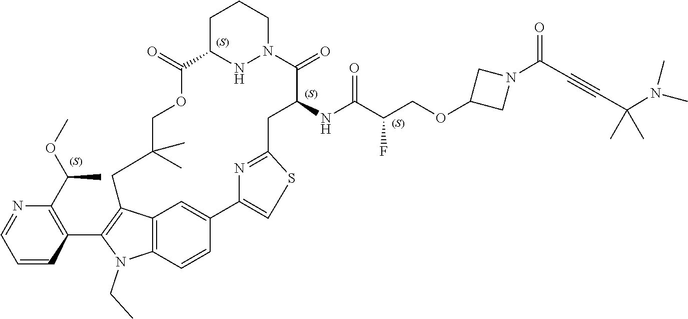 |
| BA627 | 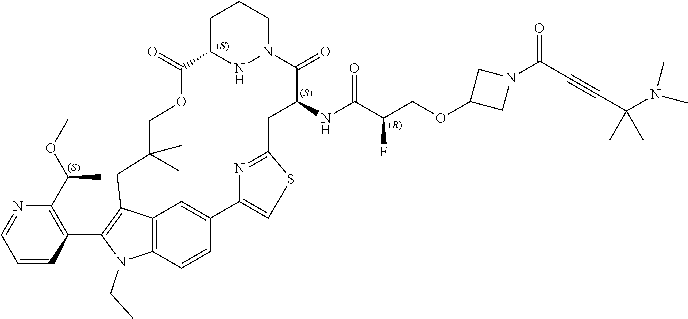 |
| BA628 | 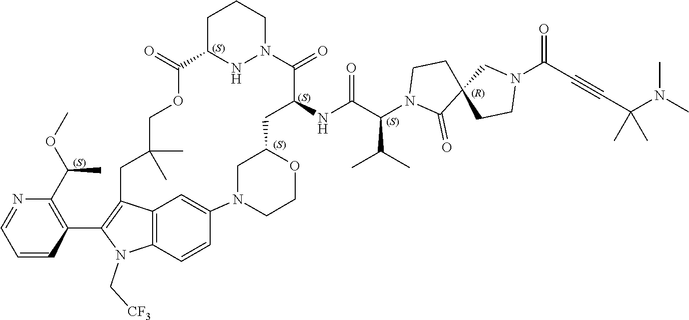 |
| BA629 | 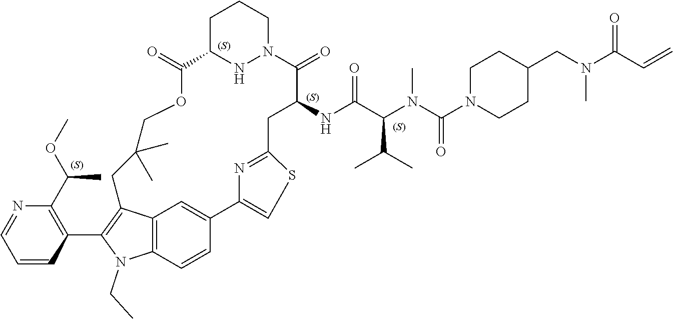 |
| BA630 | 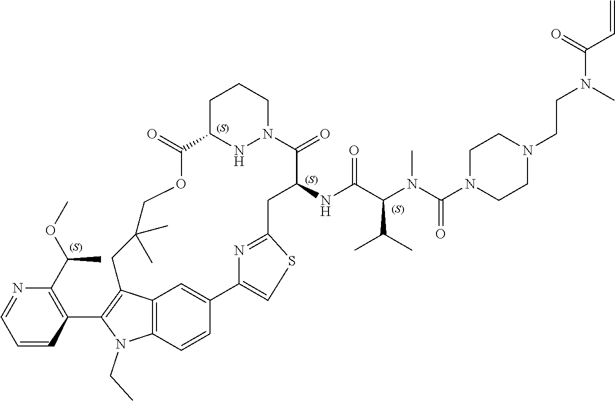 |
| BA631 | 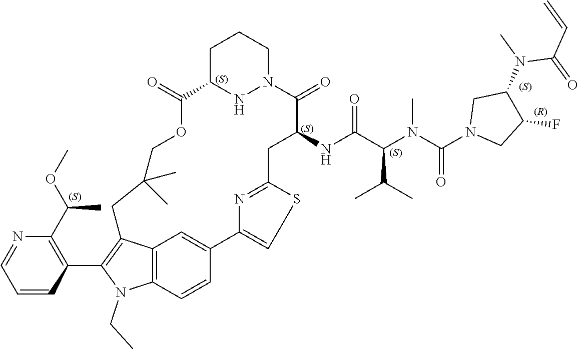 |
| BA632 | 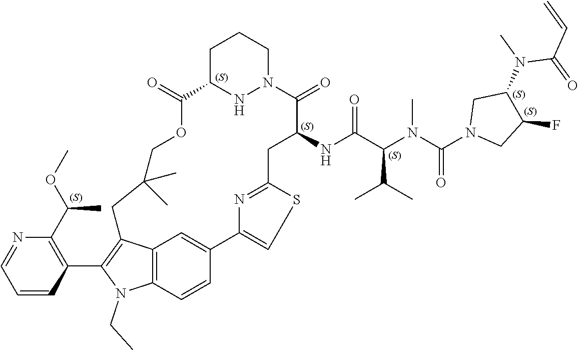 |
| BA633 | 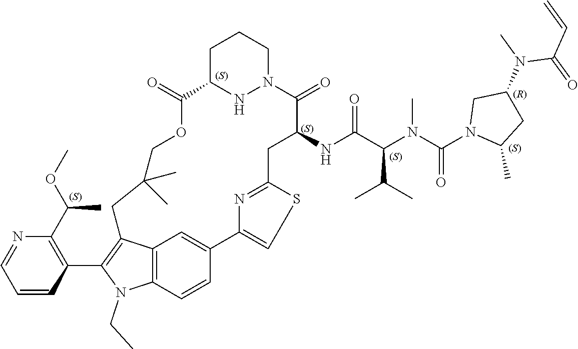 |
| BA634 | 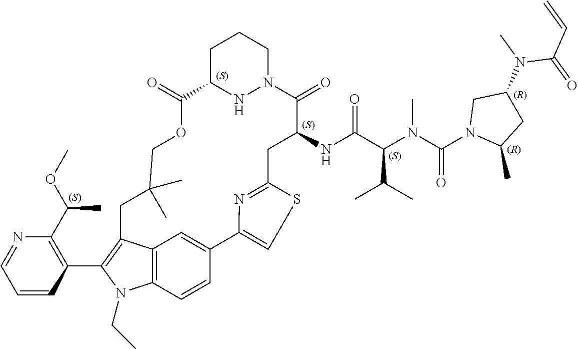 |
| BA635 | 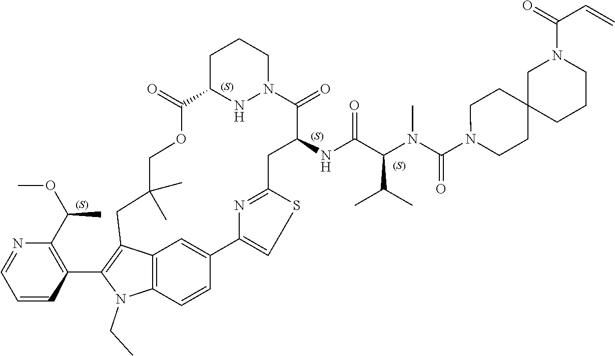 |
| BA636 | 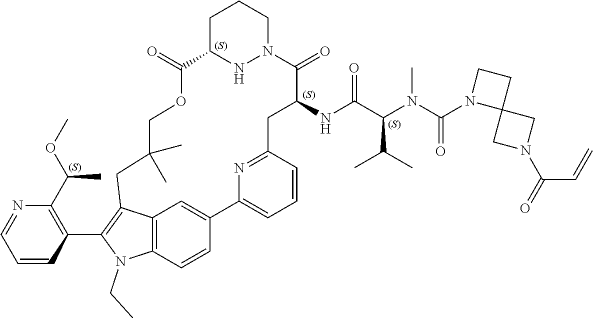 |
| BA637 | 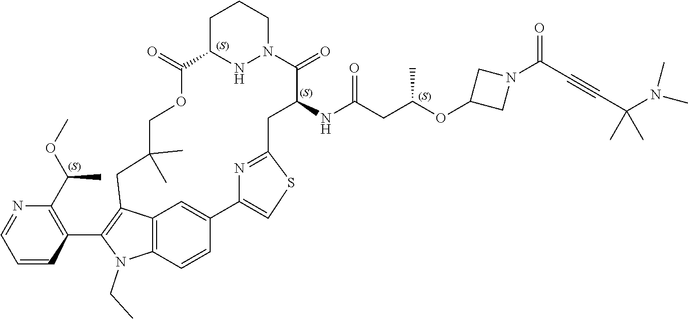 |
| BA638 | 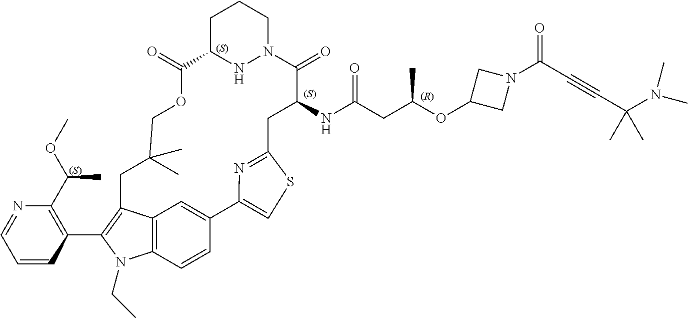 |
| BA639 | 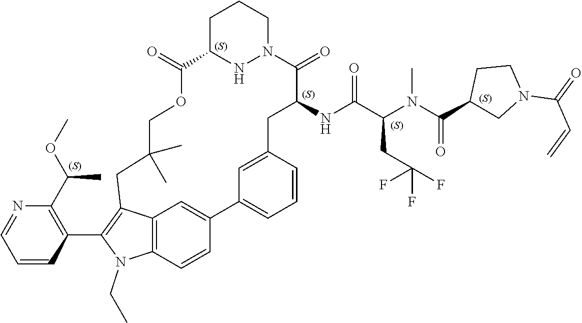 |
| BA640 | 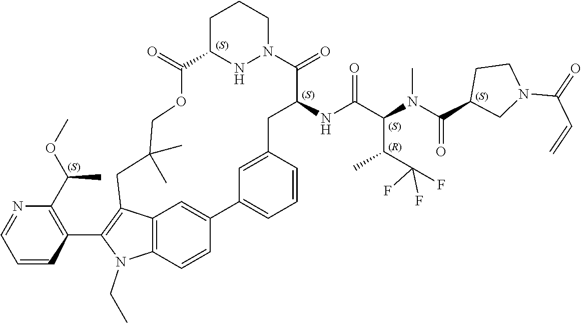 |
| BA641 | 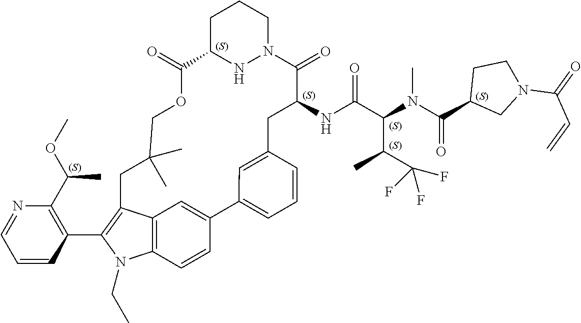 |
| BA642 | 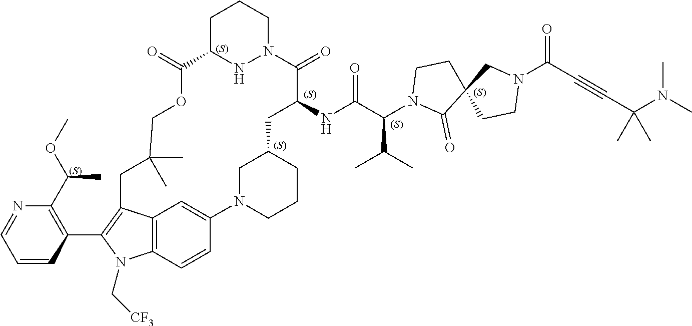 |
| BA643 | 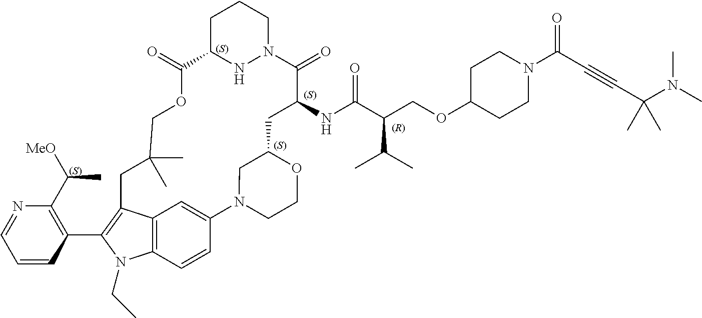 |
| BA644 | 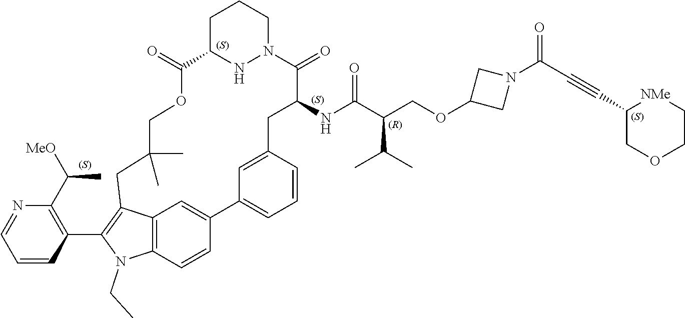 |
| BA645 | 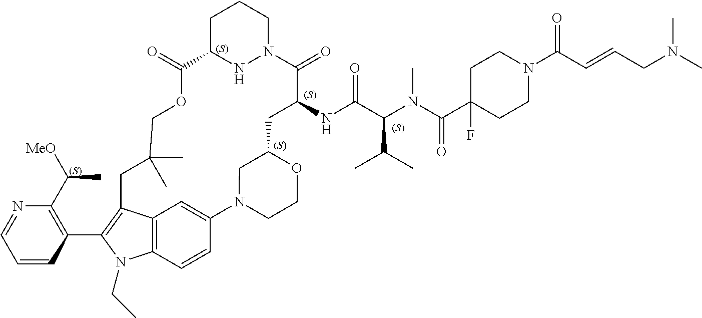 |
| BA646 | 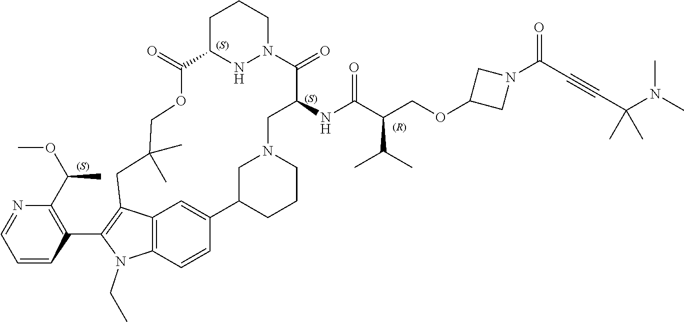 |
| BA647 | 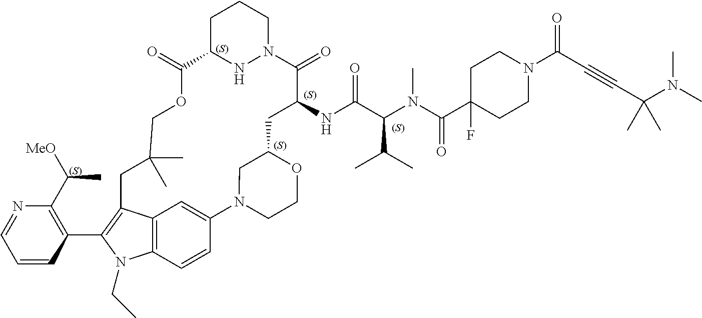 |
| BA648 | 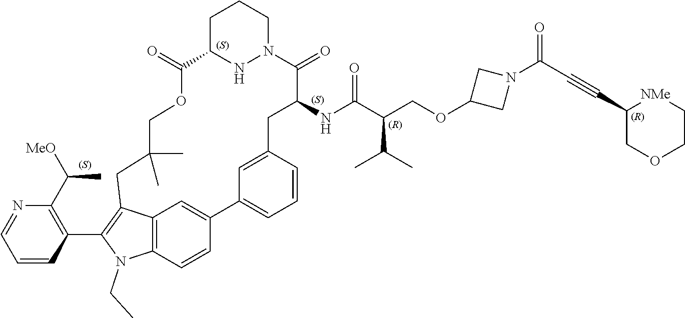 |
| BA649 | 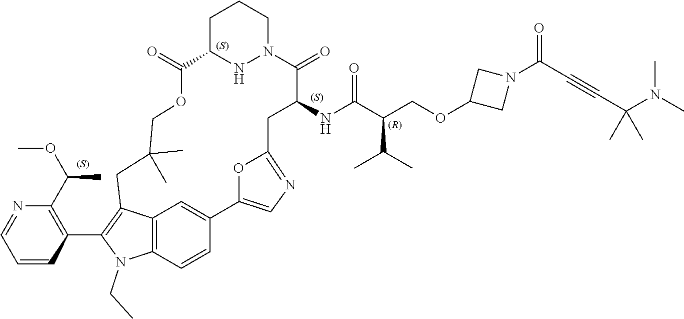 |
| BA650 | 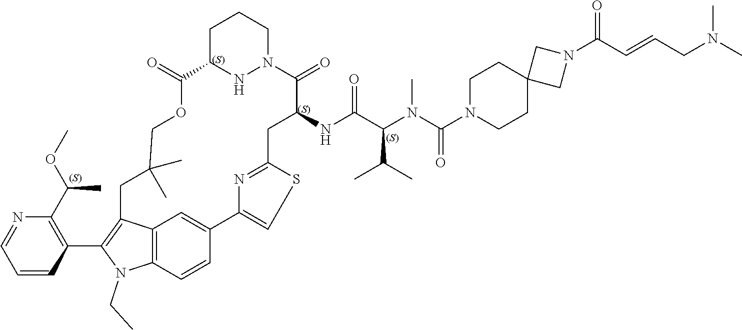 |
| BA651 | 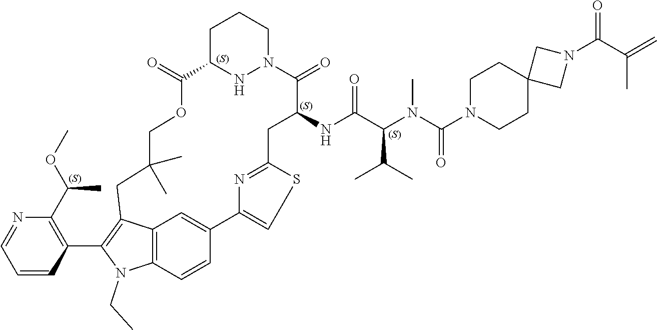 |
| BA652 | 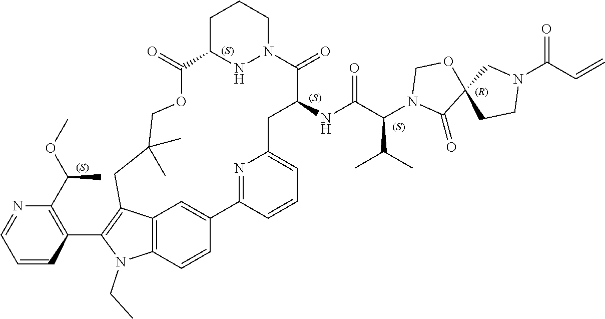 |
| BA653 | 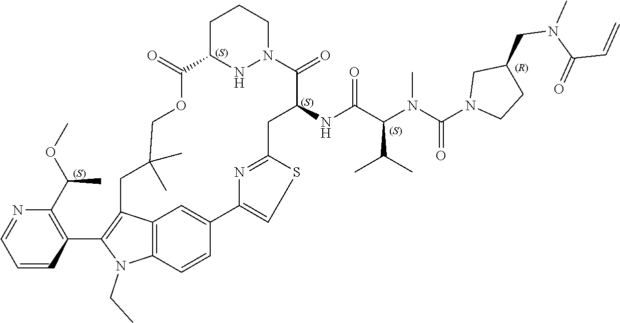 |
| BA654 | 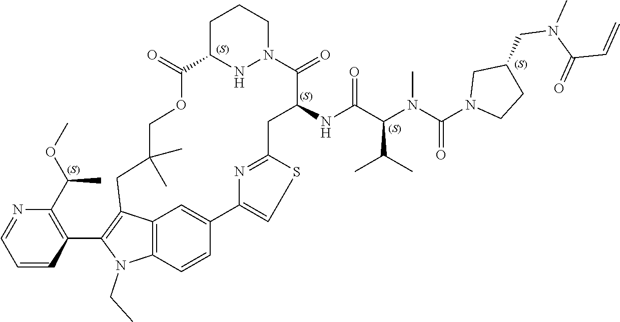 |
| BA655 | 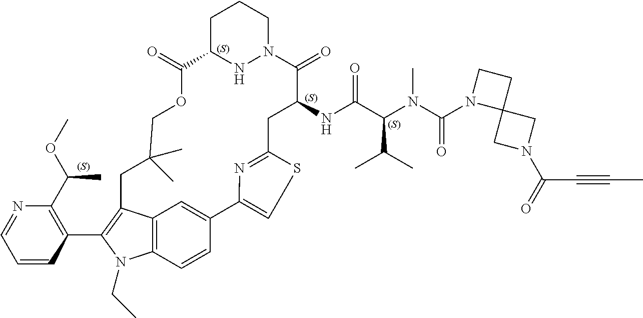 |
| BA656 | 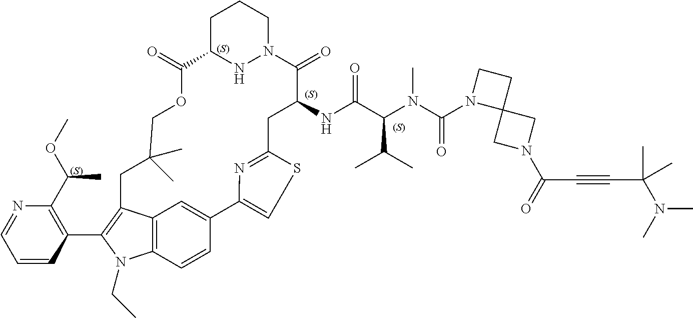 |
| BA657 | 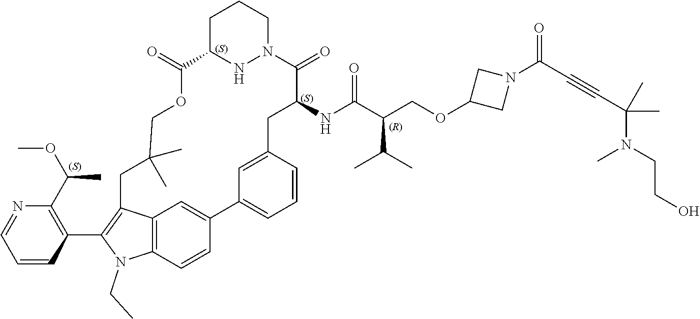 |
| BA658 | 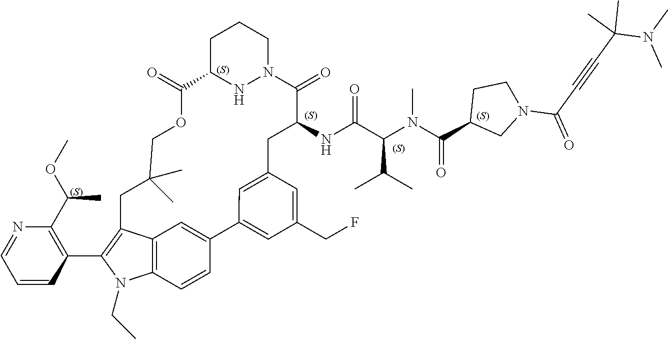 |
| BA659 | 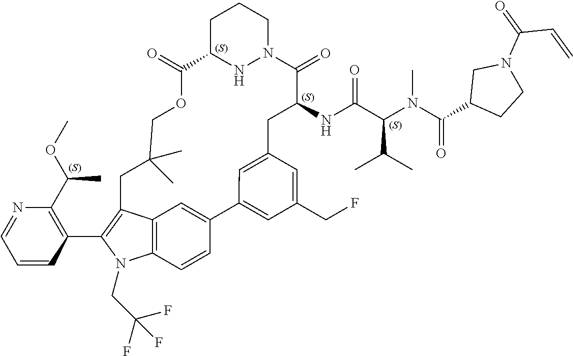 |
| BA660 | 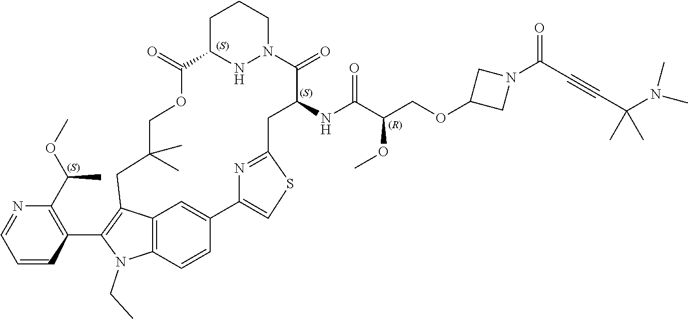 |
| BA661 | 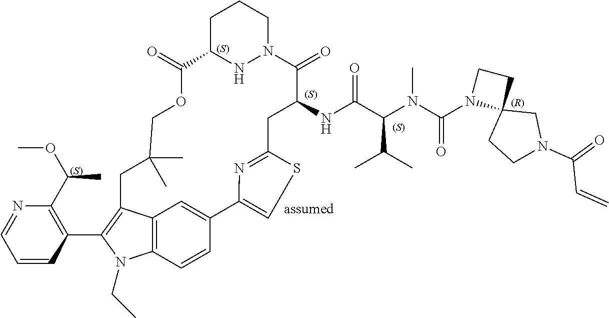 |
| BA662 | 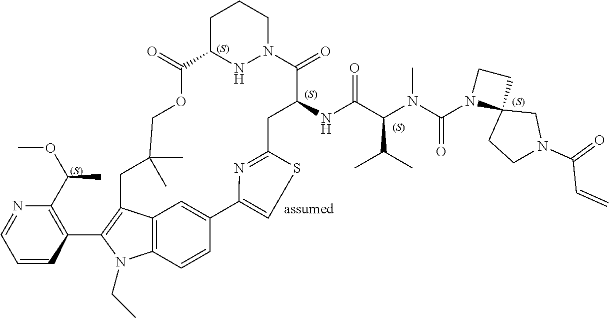 |
| BA663 | 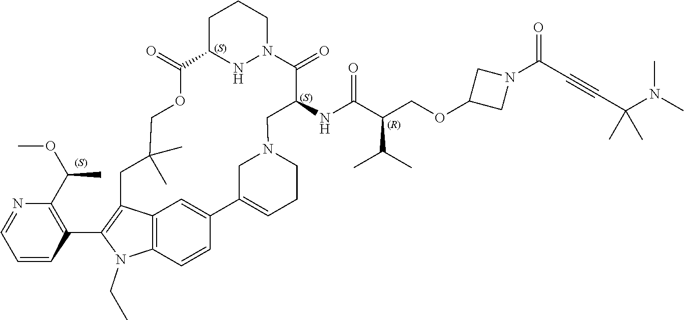 |
| BA664 | 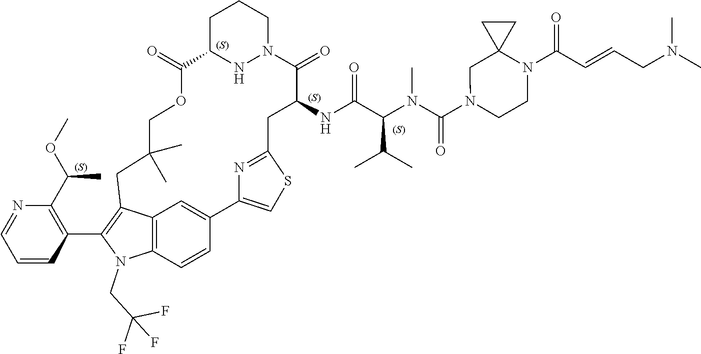 |
| BA665 | 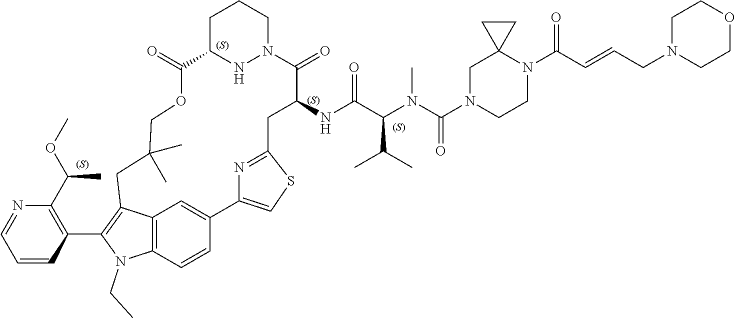 |
| BA666 | 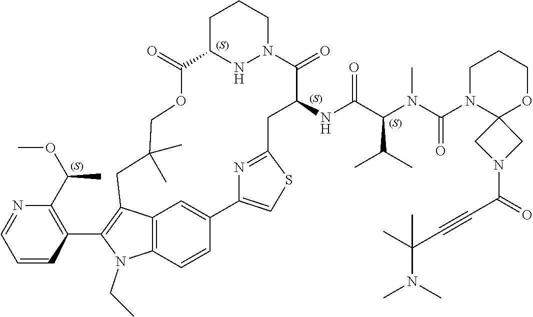 |
| BA667 | 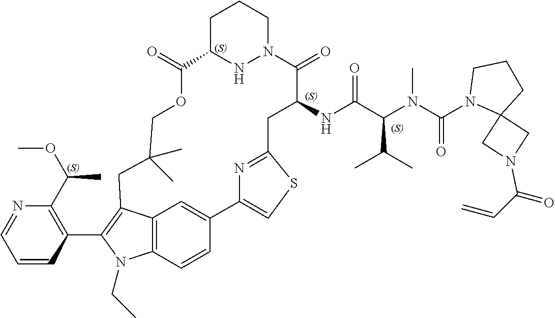 |
| BA668 | 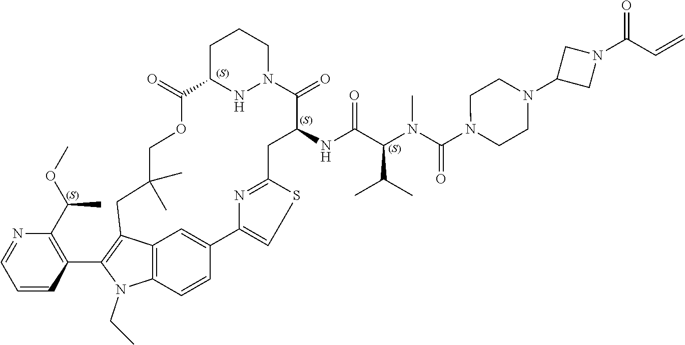 |
| BA669 | 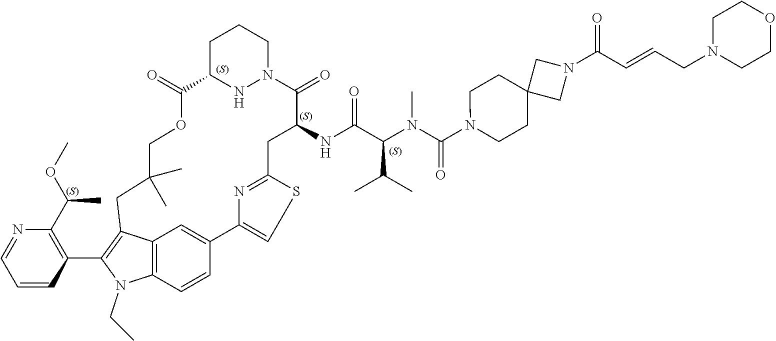 |
| BA670 | 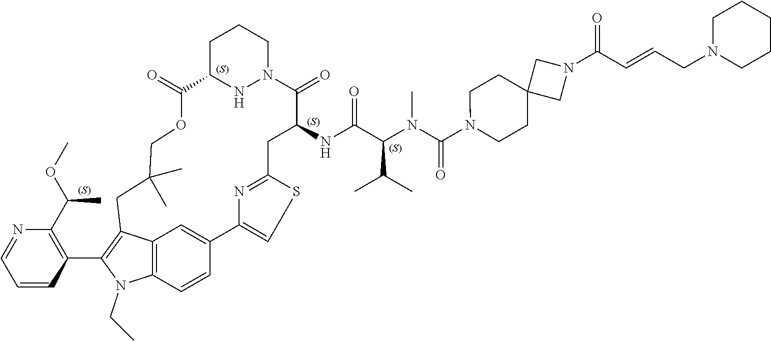 |
| BA671 | 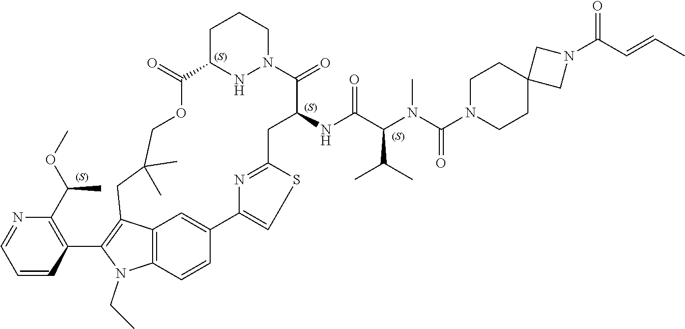 |
| BA672 | 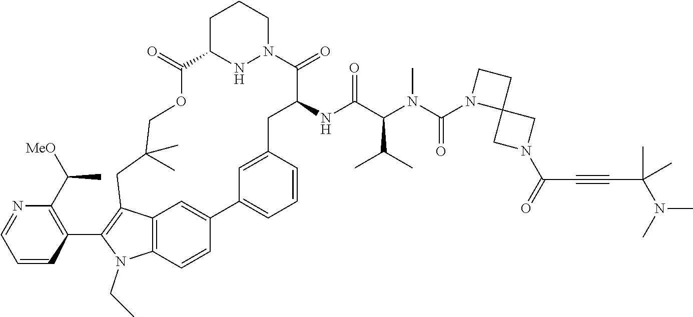 |
| BA673 | 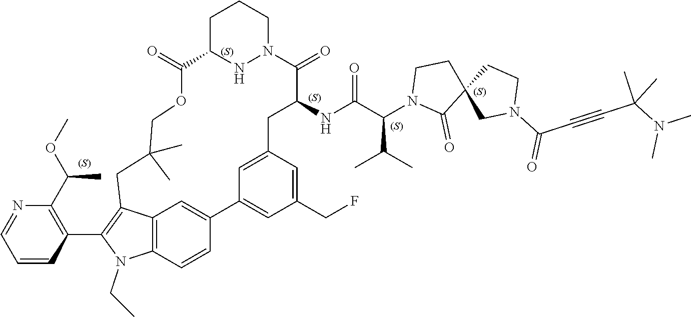 |
| BA674 | 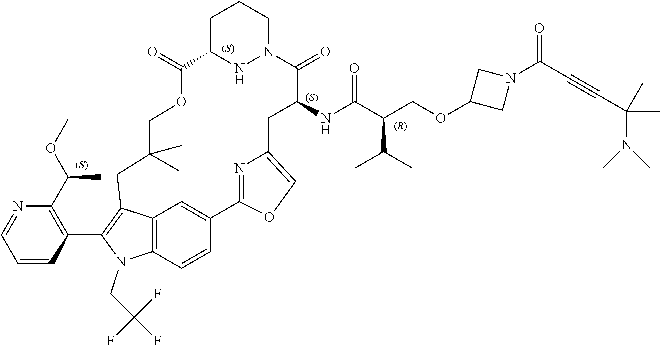 |
| BA675 | 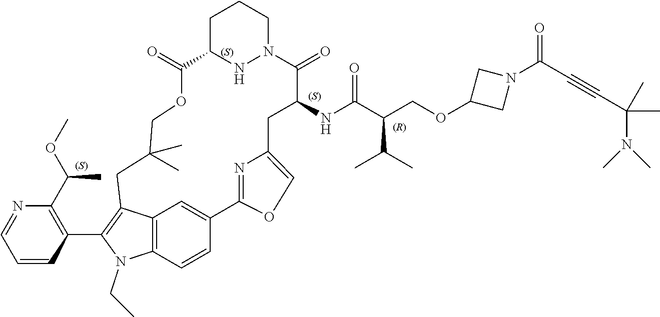 |
| BA676 | 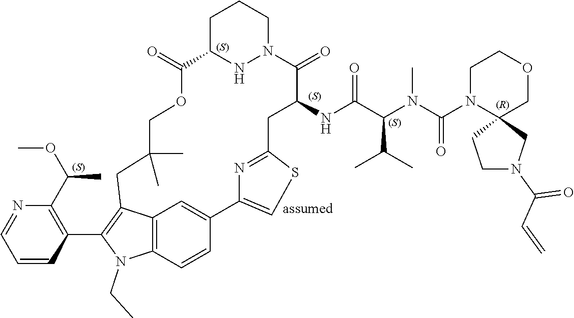 |
| BA677 | 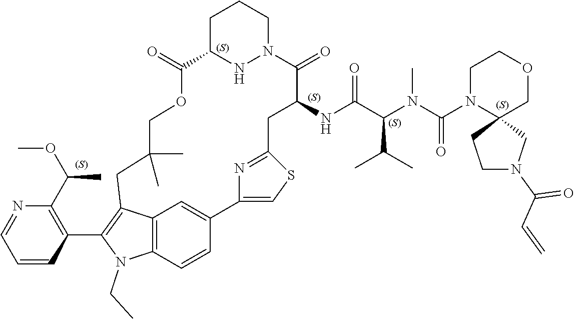 |
| BA678 | 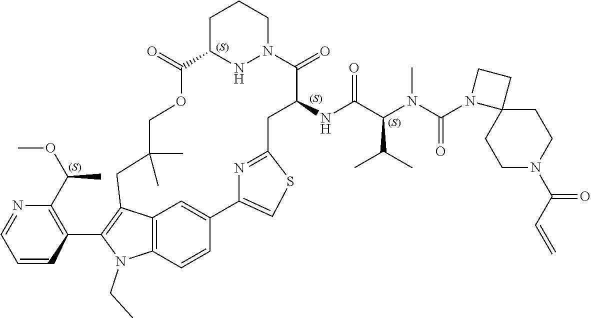 |
| BA679 | 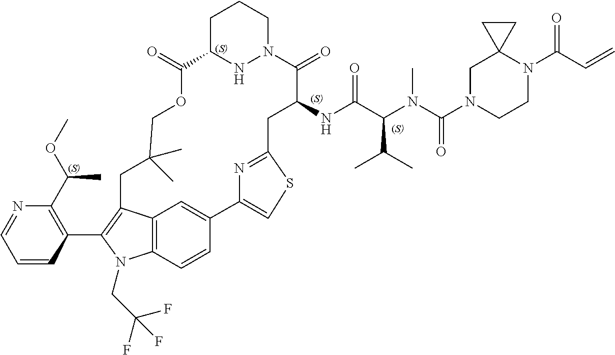 |
| BA680 | 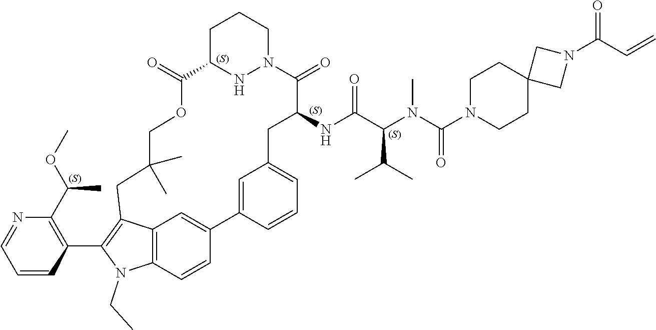 |
| BA681 | 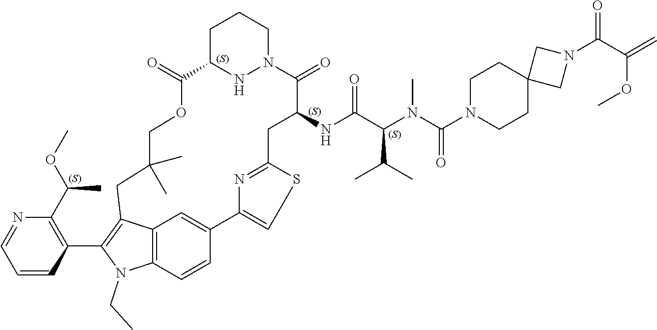 |
| BA682 | 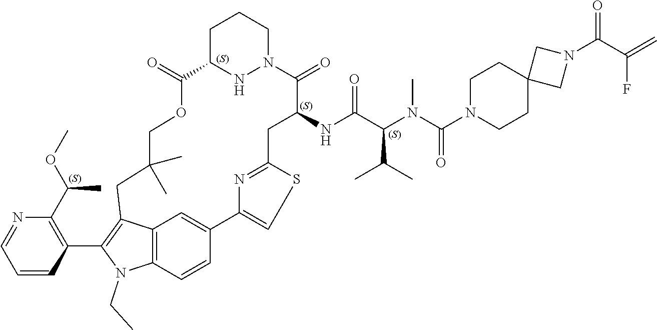 |
| BA683 | 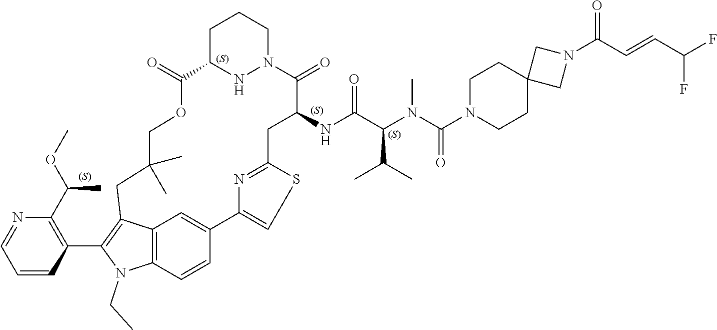 |
| BA684 | 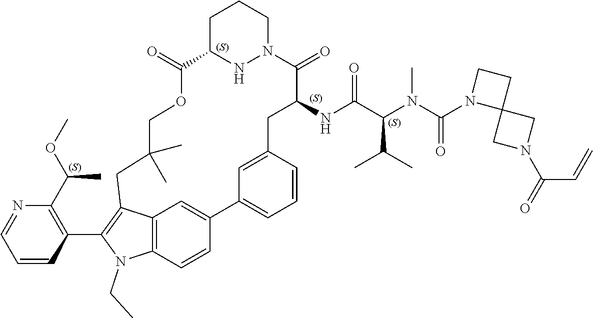 |
| BA685 | 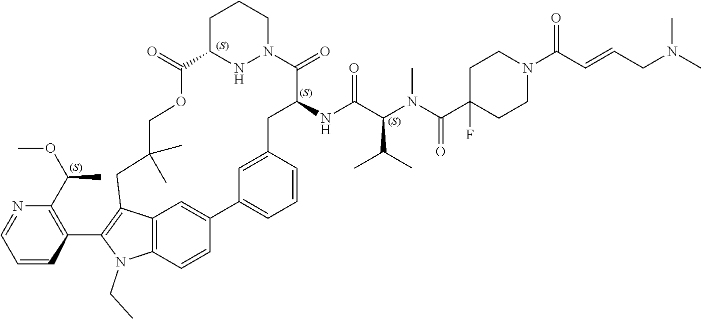 |
| BA686 | 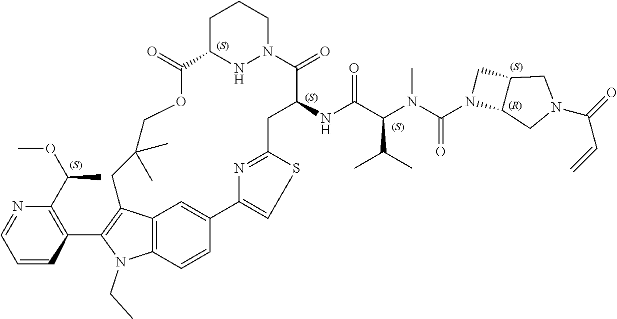 |
| BA687 | 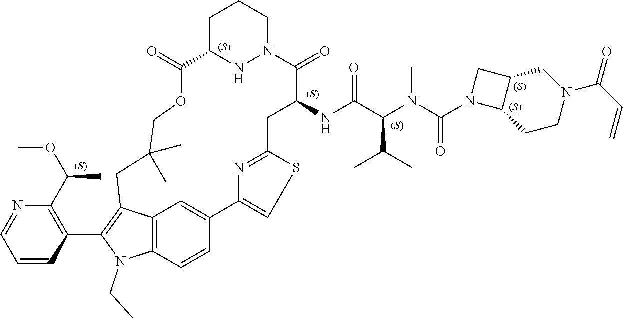 |
| BA688 | 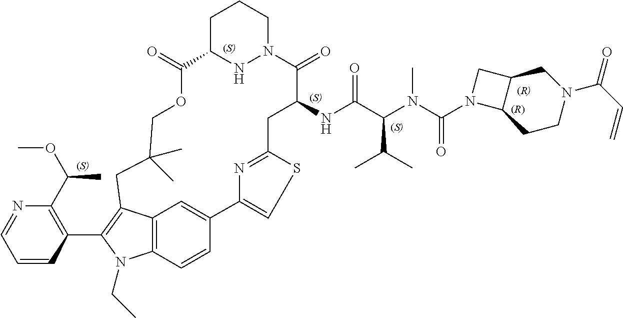 |
| BA689 | 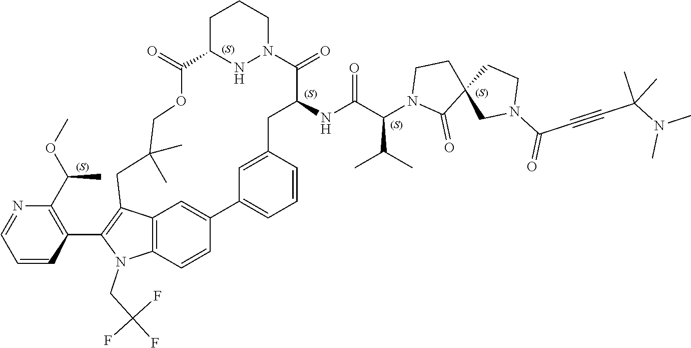 |
| BA690 | 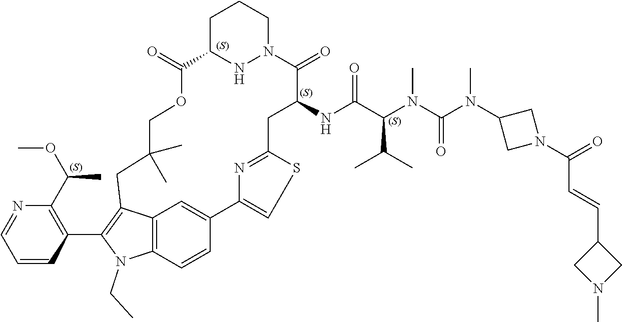 |
| BA691 | 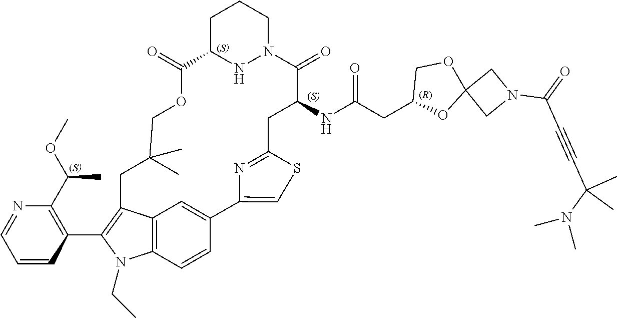 |
| BA692 | 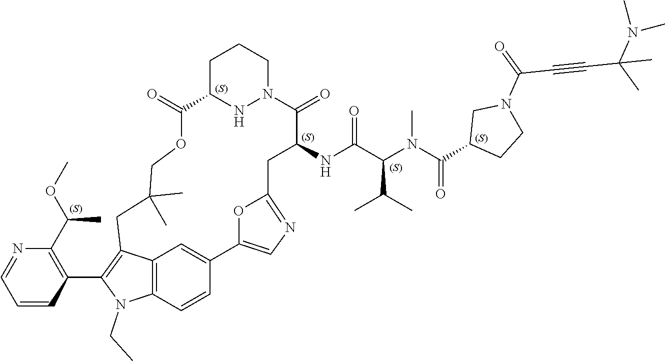 |
| BA693 | 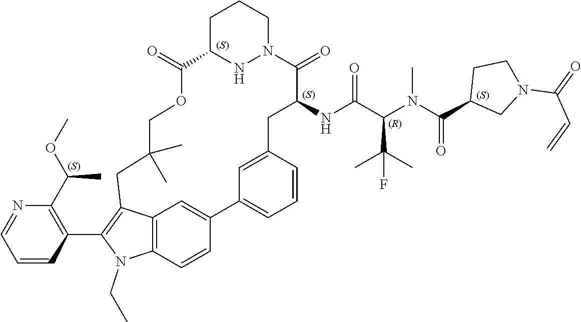 |
| BA694 | 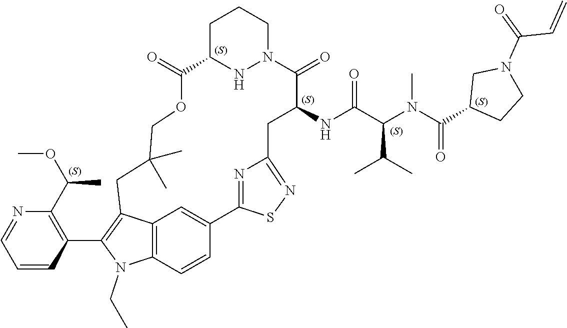 |
| BA695 | 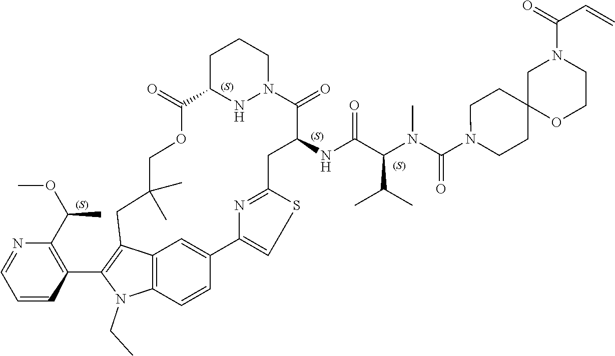 |
| BA696 | 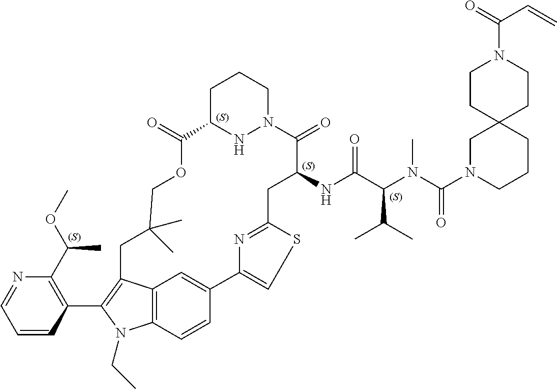 |
| BA697 | 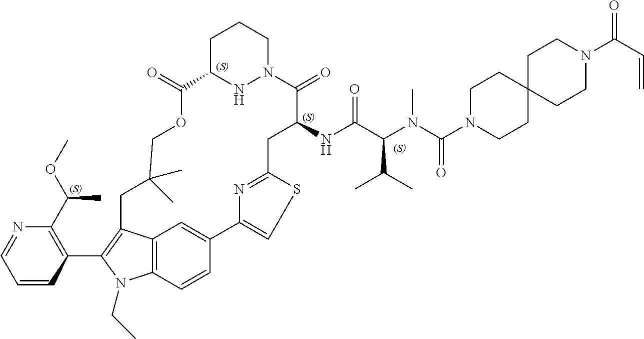 |
| BA698 | 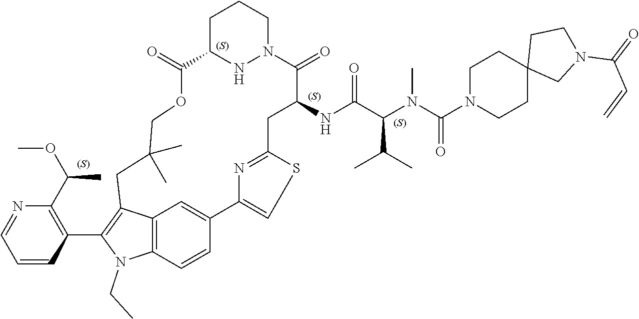 |
| BA699 | 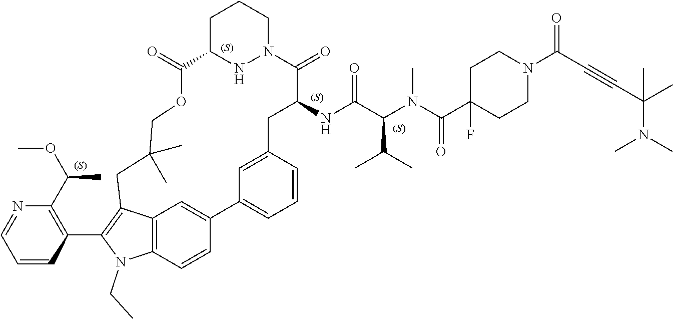 |
| BA700 | 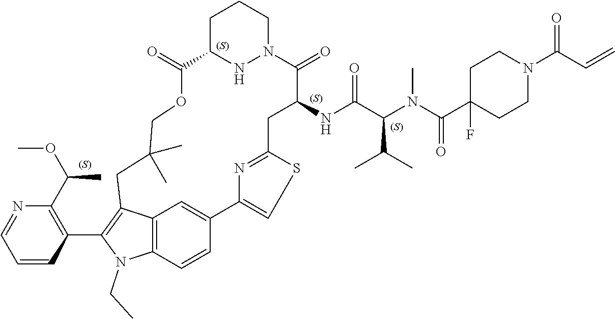 |
| BA701 | 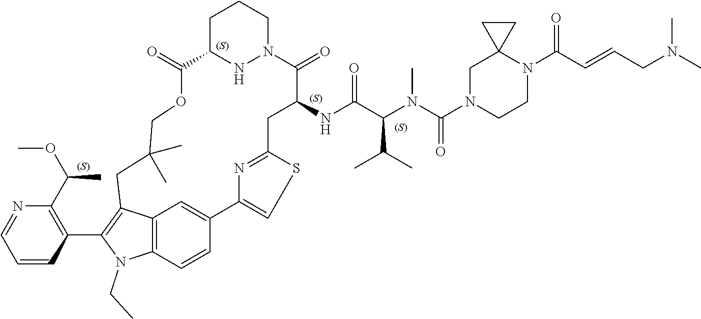 |
| BA702 | 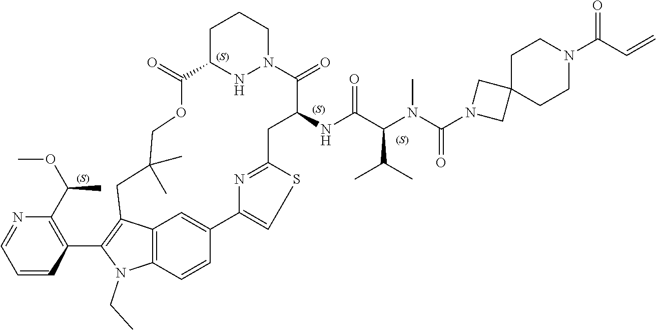 |
| BA703 | 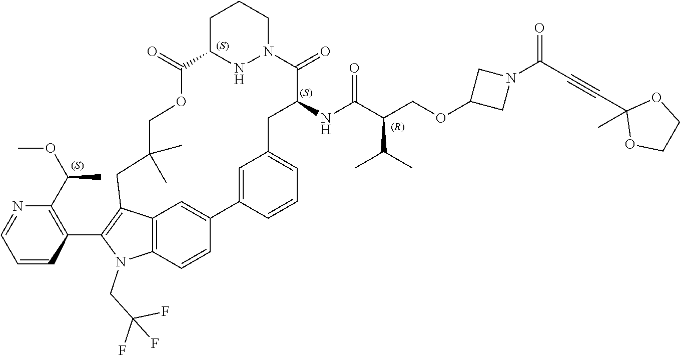 |
| BA704 | 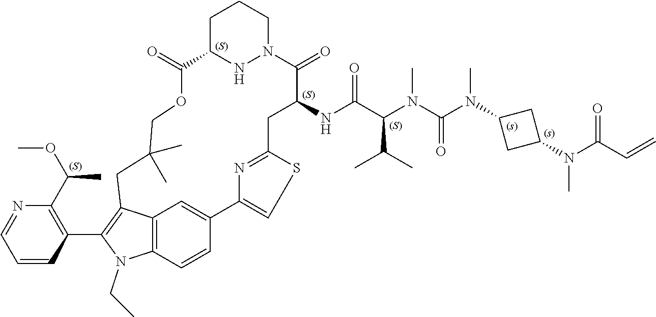 |
| BA705 | 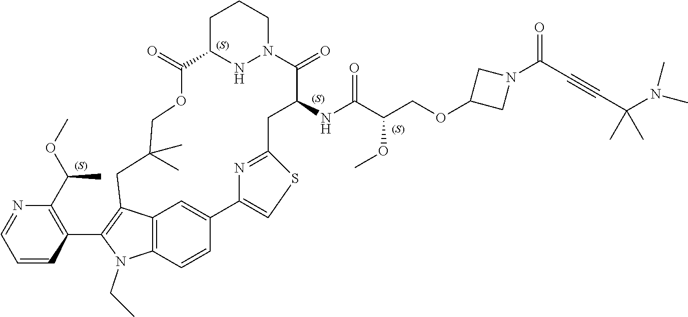 |
| BA706 | 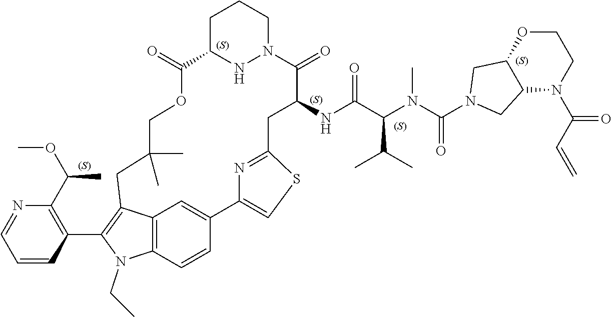 |
| BA707 | 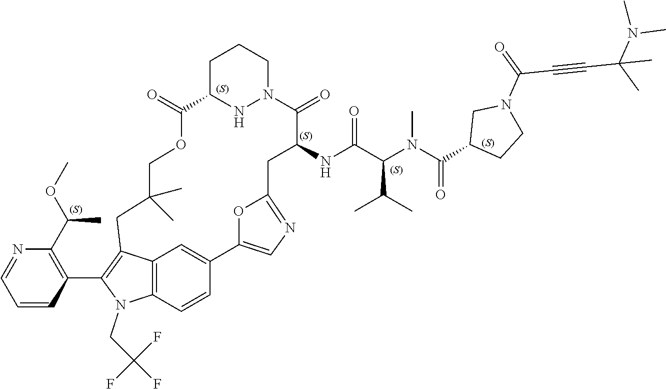 |
| BA708 | 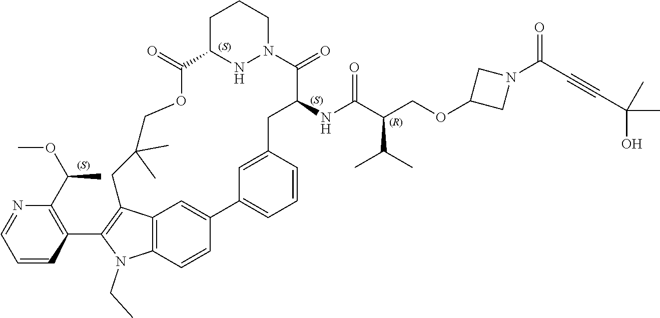 |
| BA709 | 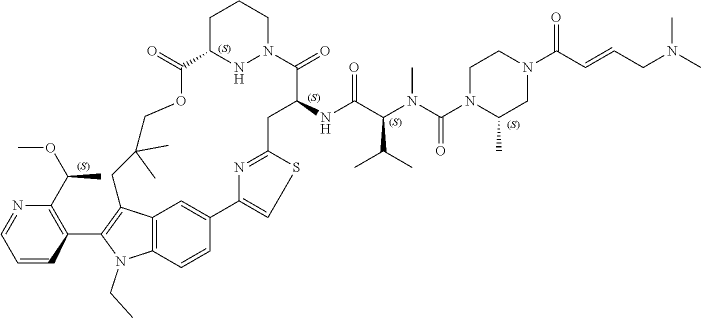 |
| BA710 | 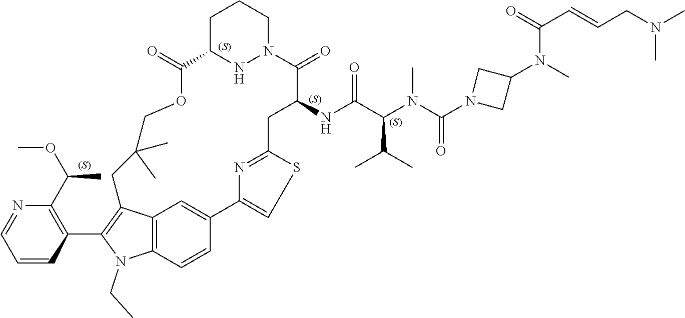 |
| BA711 | 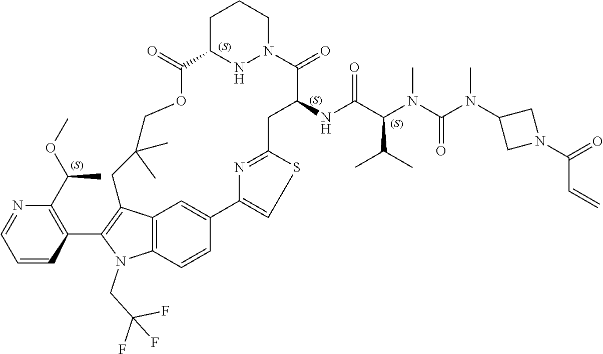 |
| BA712 | 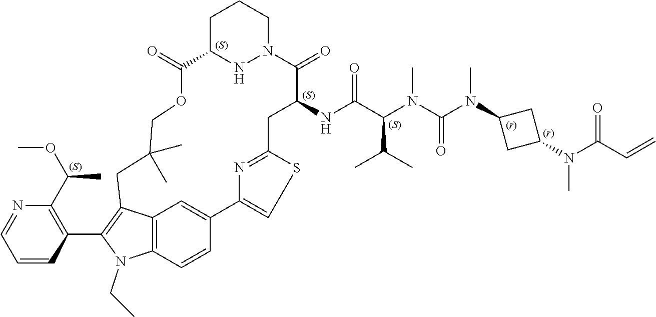 |
| BA713 | 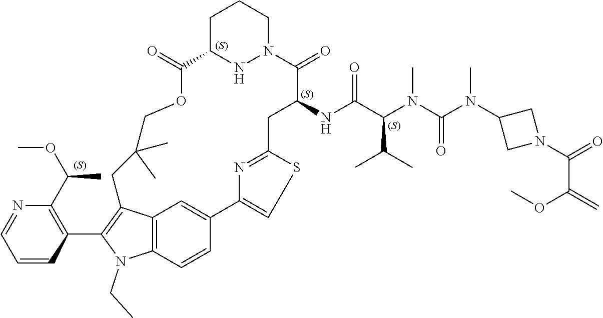 |
| BA714 | 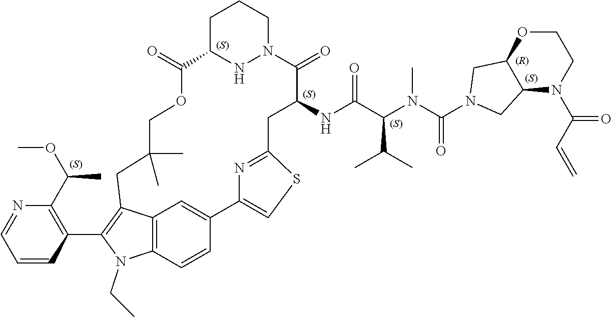 |
| BA715 | 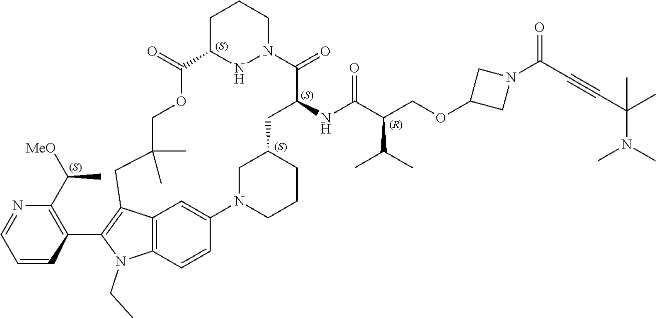 |
| BA716 | 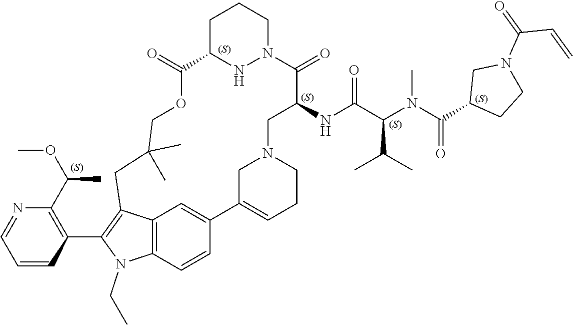 |
| BA717 | 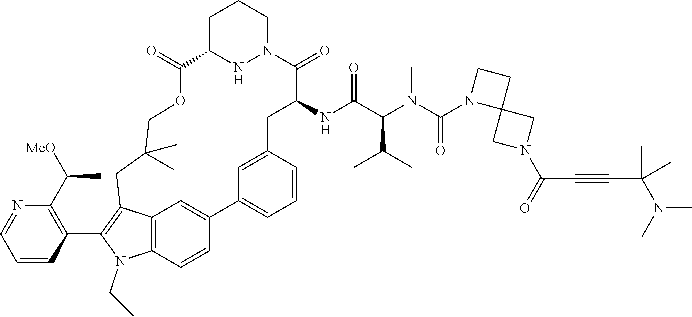 |
| BA718 | 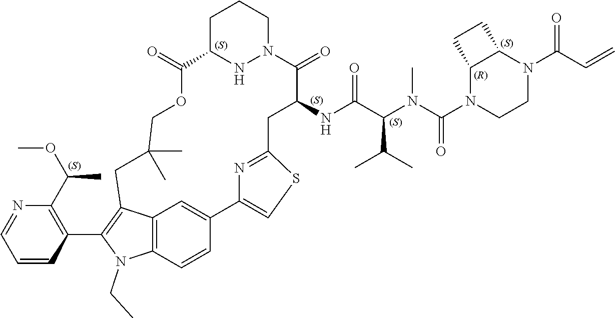 |
| BA719 | 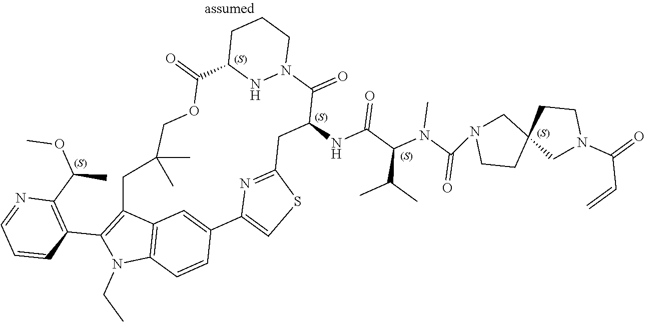 |
| BA720 | 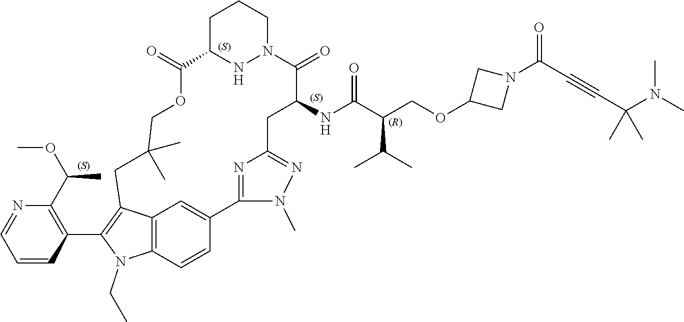 |
| BA721 | 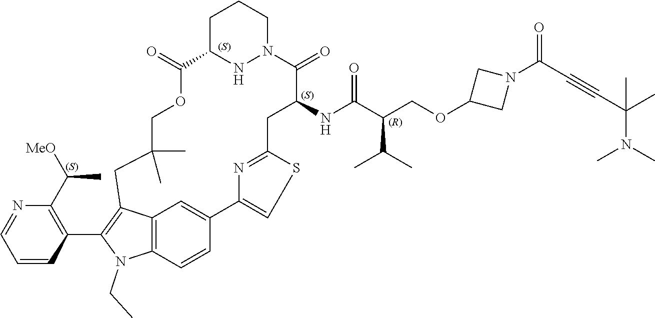 |
| BA722 | 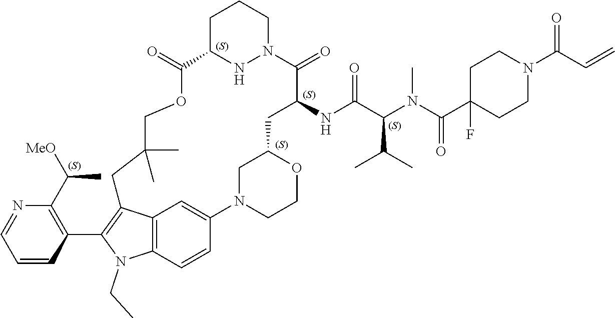 |
| BA723 | 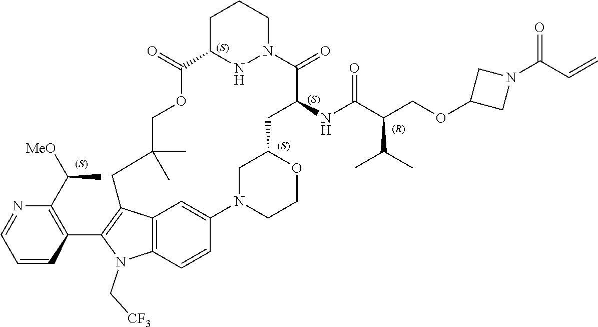 |
| BA724 | 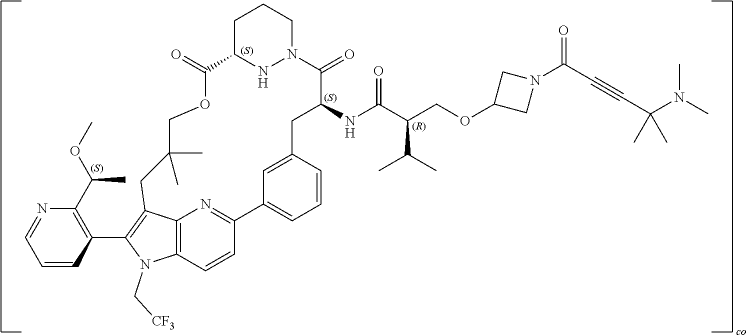 |
| BA725 | 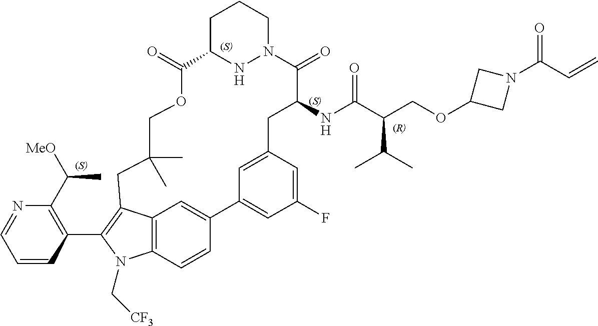 |
| BA726 | 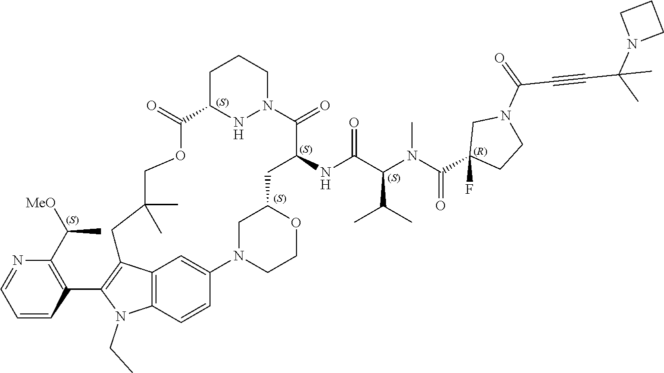 |
| BA727 | 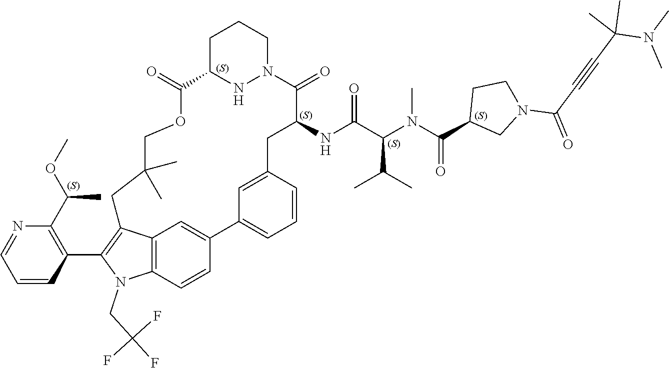 |
| BA728 | 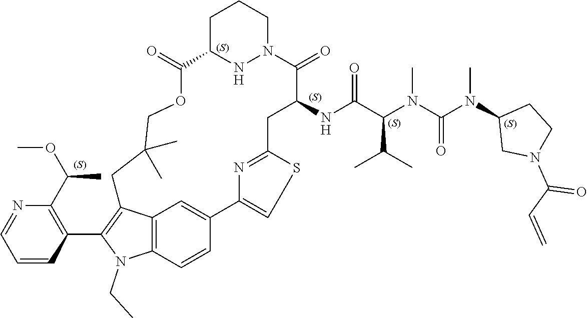 |
| BA729 | 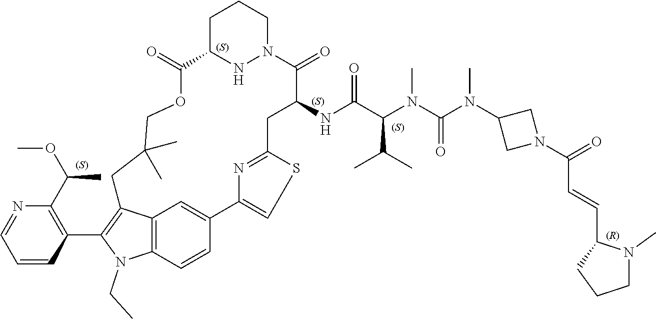 |
| BA730 | 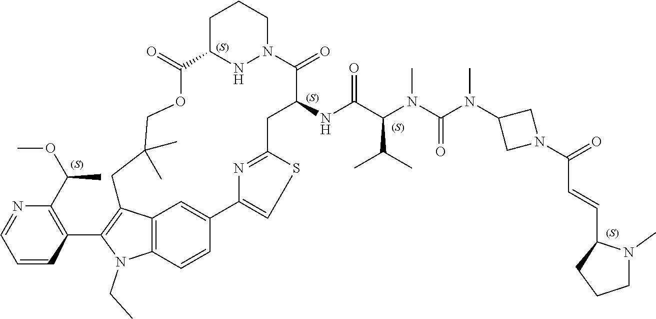 |
| BA731 | 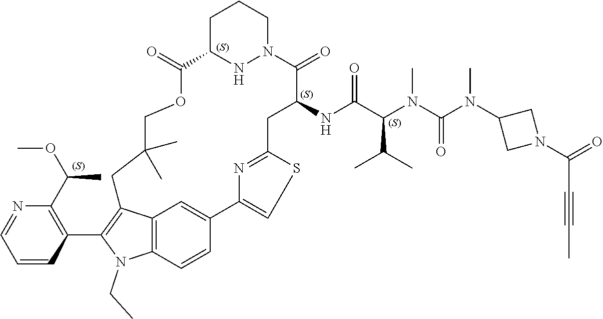 |
| BA732 | 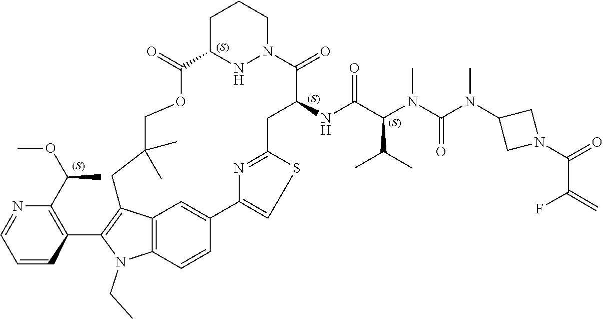 |
| BA733 | 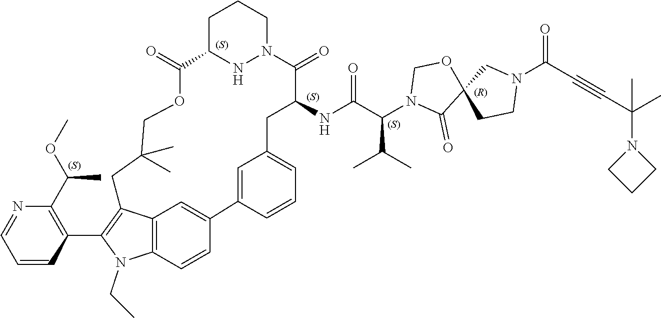 |
| BA734 | 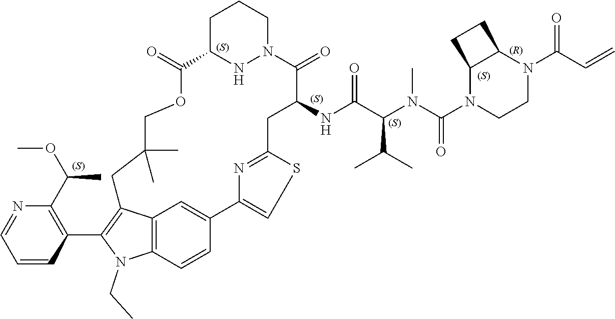 |
| BA735 | 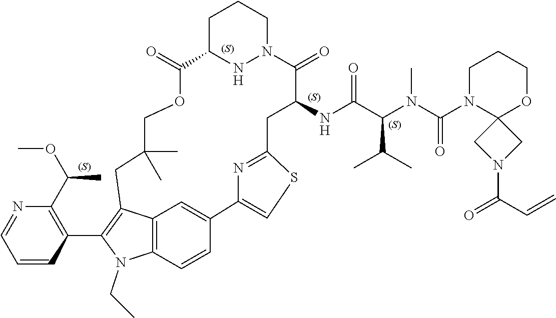 |
| BA736 | 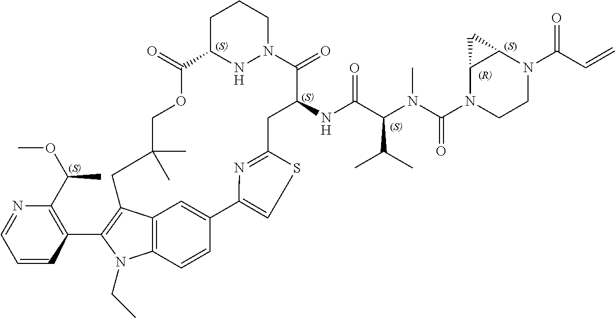 |
| BA737 | 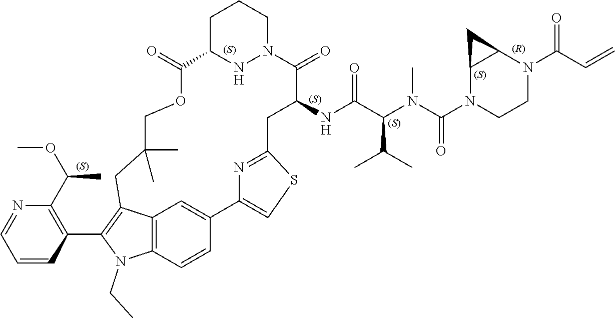 |
| BA738 | 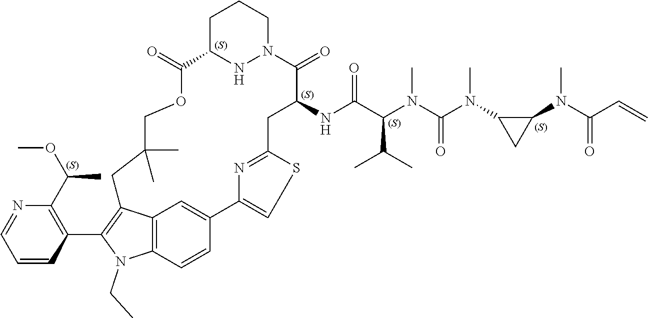 |
| BA739 | 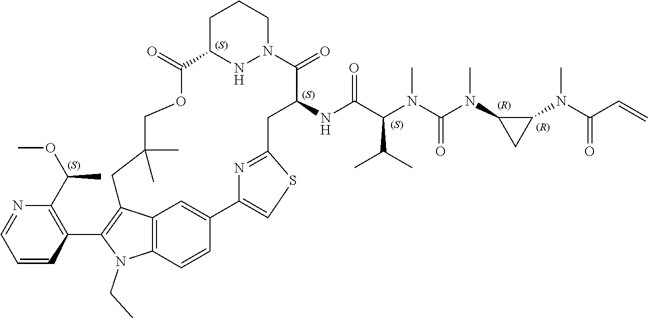 |
| BA740 | 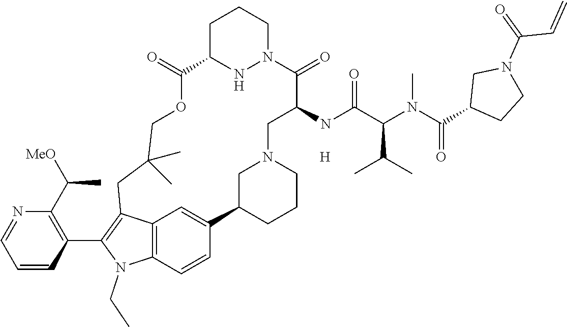 |
| BA741 | 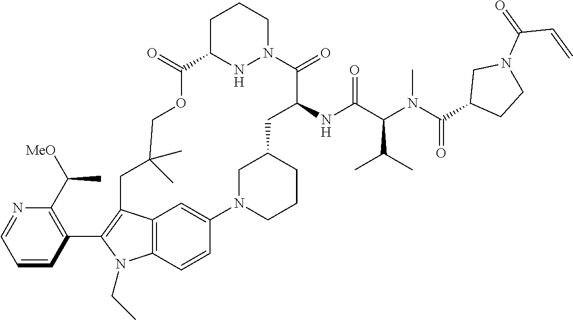 |

Note
that some compounds are shown with bonds as flat or wedged.
In some instances, the relative stereochemistry of stereoisomers has been determined; in some instances, the absolute stereochemistry has been determined.
In some instances, a single Example number corresponds to a mixture of stereoisomers.
All stereoisomers of the compounds of the foregoing table are contemplated by the present invention.
In particular embodiments, an atropisomer of a compound of the foregoing table is contemplated.
Brackets are to be ignored.
*The activity of this stereoisomer may, in fact, be attributable to the presence of a small amount of the stereoisomer with the (S) configuration at the —NC(O)—CH(CH₃)₂—N(CH₃)— position.

In some embodiments, the RAS G12C inhibitor is a compound of Table B2, or a pharmaceutically acceptable salt thereof. In some embodiments, the RAS G12C inhibitor is selected from Table B2, or a pharmaceutically acceptable salt or atropisomer thereof.

TABLE B2
Certain Compounds of the Present Invention

| Ex# | Structure |
|---|---|
| BB 1 | 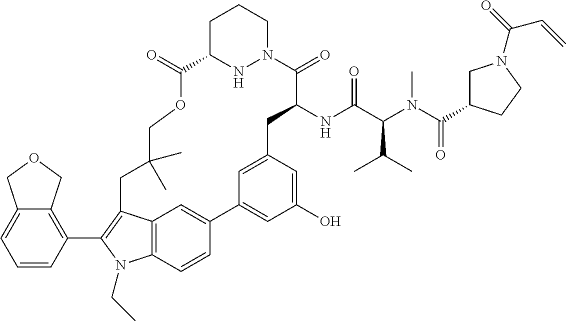 [SMILES: C=CC(=O)N1CC[C@H](C(=O)N(C)[C@H](C(=O)N[C@H]2Cc3cc(O)cc(c3)-c3ccc4c(c3)c(c(-c3cccc5c3COC5)n4CC)CC(C)(C)COC(=O)[C@@H]3CCCN(N3)C2=O)C(C)C)C1] |
| BB 2 | 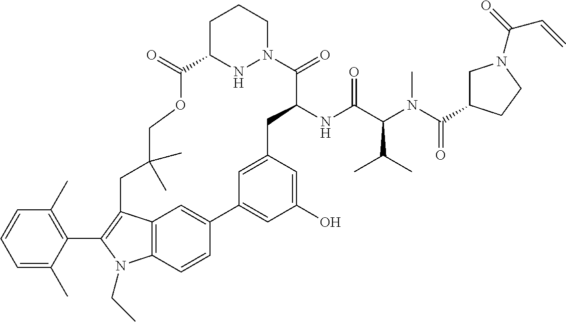 [SMILES: C=CC(=O)N1CC[C@H](C(=O)N(C)[C@H](C(=O)N[C@H]2Cc3cc(O)cc(c3)-c3ccc4c(c3)c(c(-c3c(C)cccc3C)n4CC)CC(C)(C)COC(=O)[C@@H]3CCCN(N3)C2=O)C(C)C)C1] |
| BB 3 | 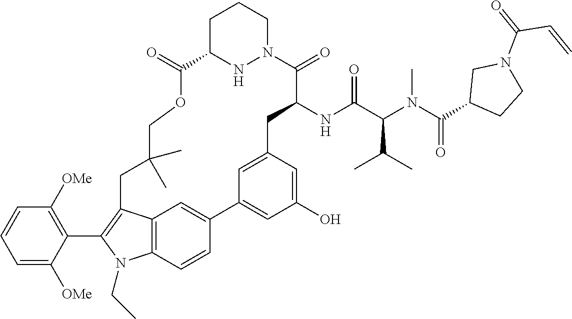 [SMILES: C=CC(=O)N1CC[C@H](C(=O)N(C)[C@H](C(=O)N[C@H]2Cc3cc(O)cc(c3)-c3ccc4c(c3)c(c(-c3c(OC)cccc3OC)n4CC)CC(C)(C)COC(=O)[C@@H]3CCCN(N3)C2=O)C(C)C)C1] |
| BB 4 | 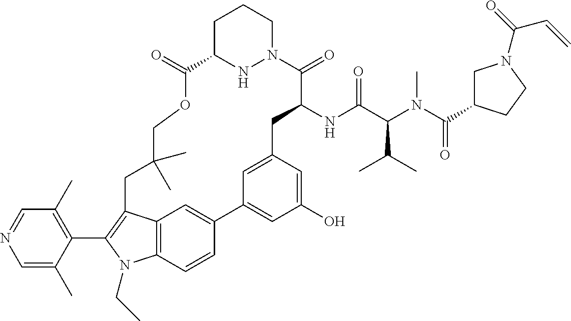 [SMILES: C=CC(=O)N1CC[C@H](C(=O)N(C)[C@H](C(=O)N[C@H]2Cc3cc(O)cc(c3)-c3ccc4c(c3)c(c(-c3c(C)cncc3C)n4CC)CC(C)(C)COC(=O)[C@@H]3CCCN(N3)C2=O)C(C)C)C1] |
| BB 5 | 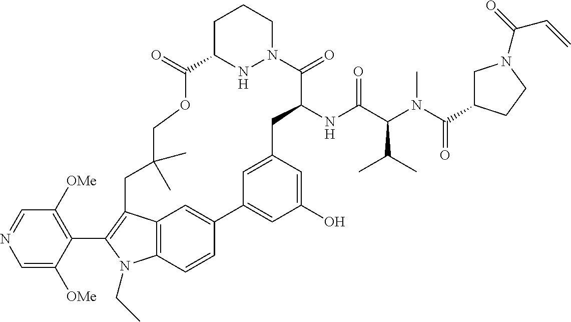 [SMILES: C=CC(=O)N1CC[C@H](C(=O)N(C)[C@H](C(=O)N[C@H]2Cc3cc(O)cc(c3)-c3ccc4c(c3)c(c(-c3c(OC)cncc3OC)n4CC)CC(C)(C)COC(=O)[C@@H]3CCCN(N3)C2=O)C(C)C)C1] |
| BB 6 | 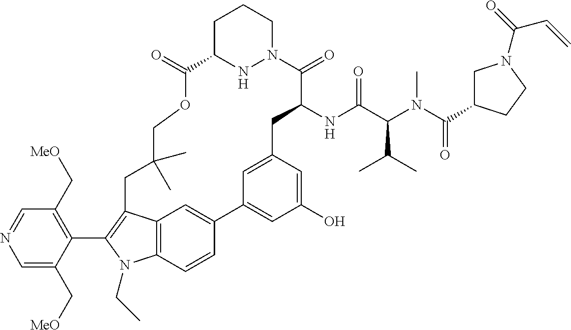 [SMILES: C=CC(=O)N1CC[C@H](C(=O)N(C)[C@H](C(=O)N[C@H]2Cc3cc(O)cc(c3)-c3ccc4c(c3)c(c(-c3c(COC)cncc3COC)n4CC)CC(C)(C)COC(=O)[C@@H]3CCCN(N3)C2=O)C(C)C)C1] |
| BB 7 | 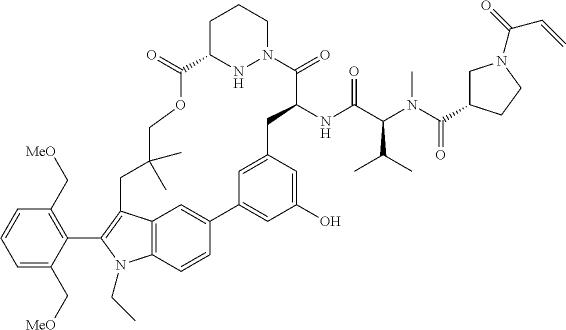 [SMILES: C=CC(=O)N1CC[C@H](C(=O)N(C)[C@H](C(=O)N[C@H]2Cc3cc(O)cc(c3)-c3ccc4c(c3)c(c(-c3c(COC)cccc3COC)n4CC)CC(C)(C)COC(=O)[C@@H]3CCCN(N3)C2=O)C(C)C)C1] |
| BB 11 | 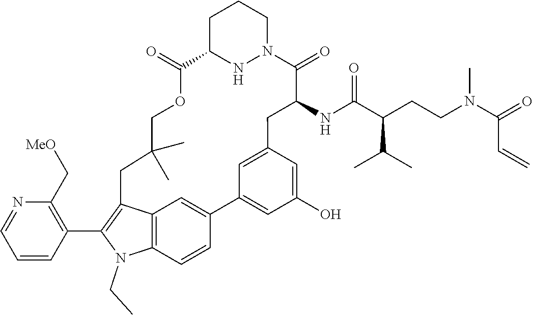 [SMILES: C=CC(=O)N(C)CC[C@H](C(=O)N[C@H]1Cc2cc(O)cc(c2)-c2ccc3c(c2)c(c(-c2cccnc2COC)n3CC)CC(C)(C)COC(=O)[C@@H]2CCCN(N2)C1=O)C(C)C] |
| BB 12 | 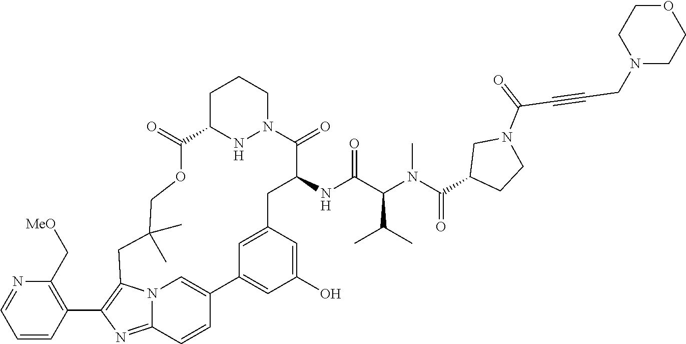 [SMILES: COCc1ncccc1-c1nc2ccc3cn2c1CC(C)(C)COC(=O)[C@@H]1CCCN(N1)C(=O)[C@@H](NC(=O)[C@H](C(C)C)N(C)C(=O)[C@H]1CCN(C(=O)C#CCN2CCOCC2)C1)Cc1cc(O)cc-3c1] |
| BB 13 | 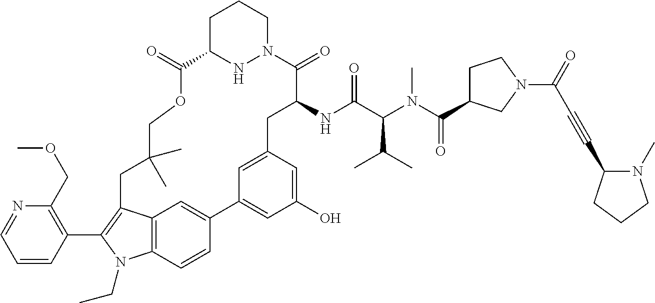 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@@H]3CCCN3C)C1)C(=O)N1CCC[C@H](N1)C(=O)OCC(C)(C)C2] |
| BB 18 | 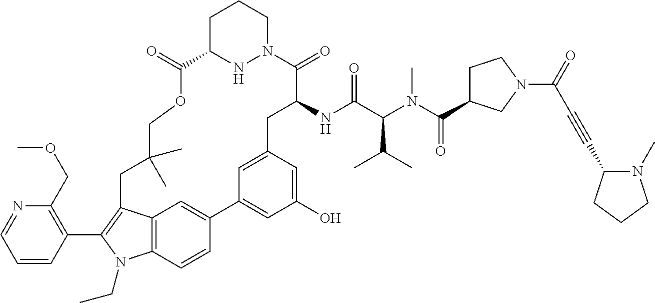 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@H]3CCCN3C)C1)C(=O)N1CCC[C@H](N1)C(=O)OCC(C)(C)C2] |
| BB 21 | 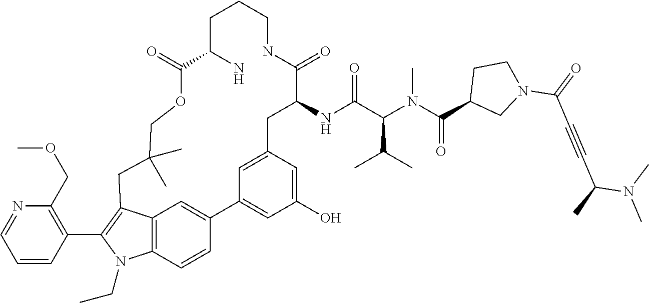 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@H](C)N(C)C)C1)C(=O)NCCC[C@H](N)C(=O)OCC(C)(C)C2] |
| BB 22 | 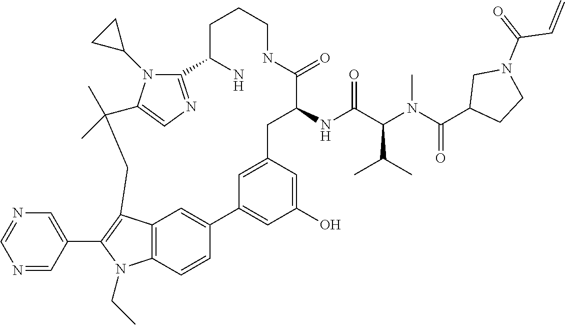 [SMILES: C=CC(=O)N1CCC(C(=O)N(C)[C@H](C(=O)N[C@H]2Cc3cc(O)cc(c3)-c3ccc4c(c3)c(c(-c3cncnc3)n4CC)CC(C)(C)c3cnc(n3C3CC3)[C@@H](N)CCCNC2=O)C(C)C)C1] |
| BB 25 | 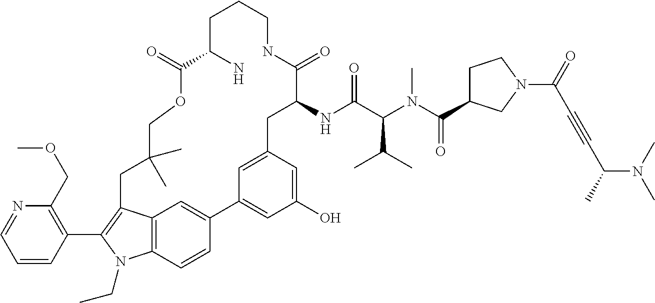 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@@H](C)N(C)C)C1)C(=O)NCCC[C@H](N)C(=O)OCC(C)(C)C2] |
| BB 27 | 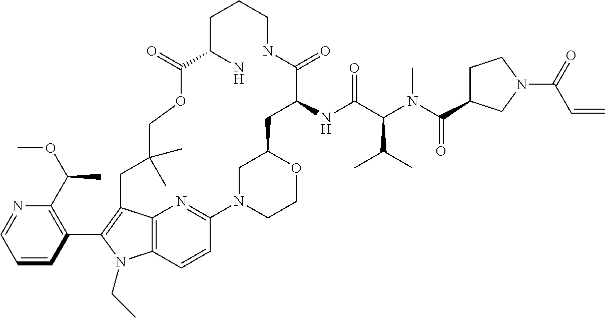 |
| BB 28 | 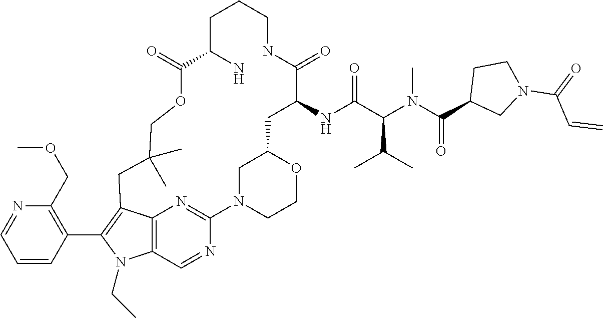 [SMILES: C=CC(=O)N1CC[C@H](C(=O)N(C)[C@H](C(=O)N[C@H]2C[C@H]3CN(CCO3)c3ncc4c(n3)c(c(-c3cccnc3COC)n4CC)CC(C)(C)COC(=O)[C@@H](N)CCCNC2=O)C(C)C)C1] |
| BB 29 | 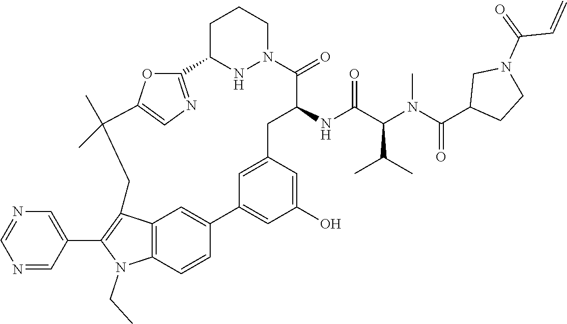 [SMILES: C=CC(=O)N1CCC(C(=O)N(C)[C@H](C(=O)N[C@H]2Cc3cc(O)cc(c3)-c3ccc4c(c3)c(c(-c3cncnc3)n4CC)CC(C)(C)C3=CN=C(O3)[C@@H]3CCCN(N3)C2=O)C(C)C)C1] |
| BB 30 | 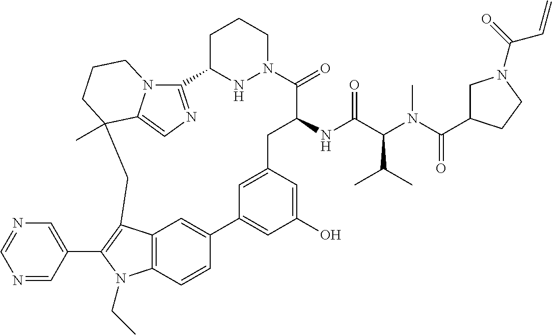 [SMILES: C=CC(=O)N1CCC(C(=O)N(C)[C@H](C(=O)N[C@H]2Cc3cc(O)cc(c3)-c3ccc4c(c3)c(c(-c3cncnc3)n4CC)CC3(C)CCCn4c3cnc4[C@@H]3CCCN(N3)C2=O)C(C)C)C1] |
| BB 32 | 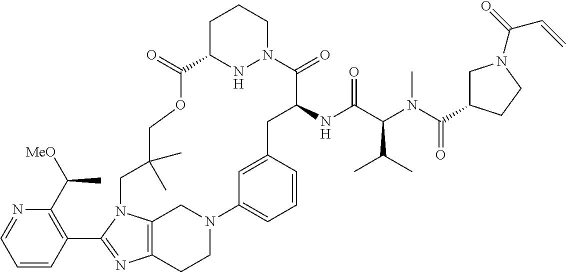 [SMILES: C=CC(=O)N1CC[C@H](C(=O)N(C)[C@H](C(=O)N[C@H]2Cc3cccc(c3)N3CCc4nc(-c5cccnc5[C@H](C)OC)n(c4C3)CC(C)(C)COC(=O)[C@@H]3CCCN(N3)C2=O)C(C)C)C1] |
| BB 34 | 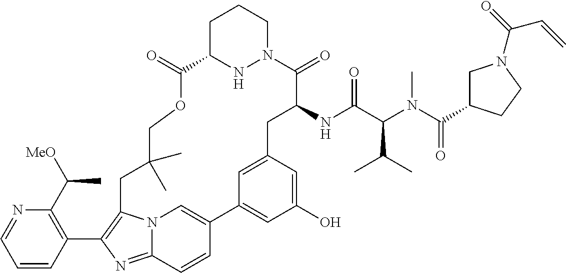 [SMILES: C=CC(=O)N1CC[C@H](C(=O)N(C)[C@H](C(=O)N[C@H]2Cc3cc(O)cc(c3)-c3ccc4nc(-c5cccnc5[C@H](C)OC)c(n4c3)CC(C)(C)COC(=O)[C@@H]3CCCN(N3)C2=O)C(C)C)C1] |
| BB 38 | 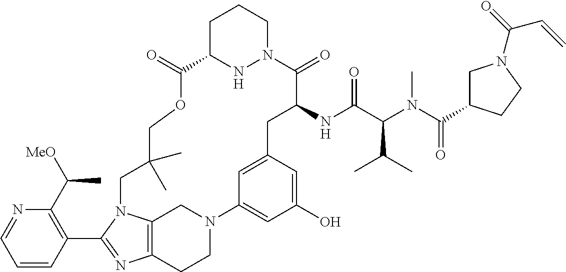 [SMILES: C=CC(=O)N1CC[C@H](C(=O)N(C)[C@H](C(=O)N[C@H]2Cc3cc(O)cc(c3)N3CCc4nc(-c5cccnc5[C@H](C)OC)n(c4C3)CC(C)(C)COC(=O)[C@@H]3CCCN(N3)C2=O)C(C)C)C1] |
| BB 47 | 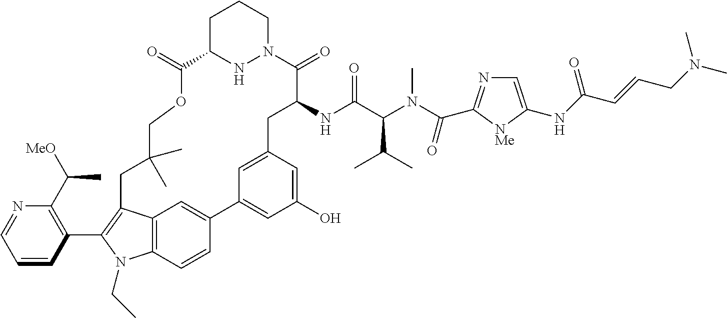 |
| BB 64 | 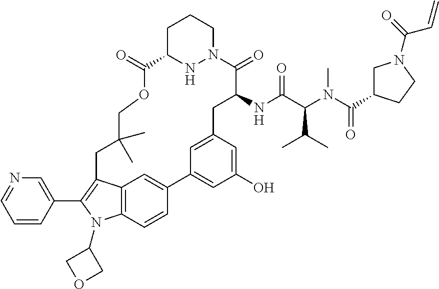 [SMILES: C=CC(=O)N1CC[C@H](C(=O)N(C)[C@H](C(=O)N[C@H]2Cc3cc(O)cc(c3)-c3ccc4c(c3)c(c(-c3cccnc3)n4C3COC3)CC(C)(C)COC(=O)[C@@H]3CCCN(N3)C2=O)C(C)C)C1] |
| BB 65 | 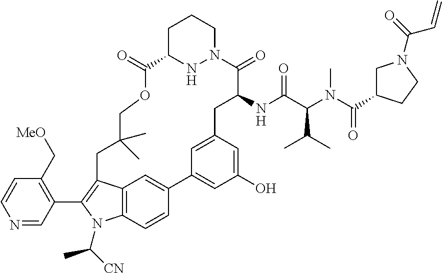 [SMILES: C=CC(=O)N1CC[C@H](C(=O)N(C)[C@H](C(=O)N[C@H]2Cc3cc(O)cc(c3)-c3ccc4c(c3)c(c(-c3cnccc3COC)n4[C@H](C)C#N)CC(C)(C)COC(=O)[C@@H]3CCCN(N3)C2=O)C(C)C)C1] |
| BB 66 | 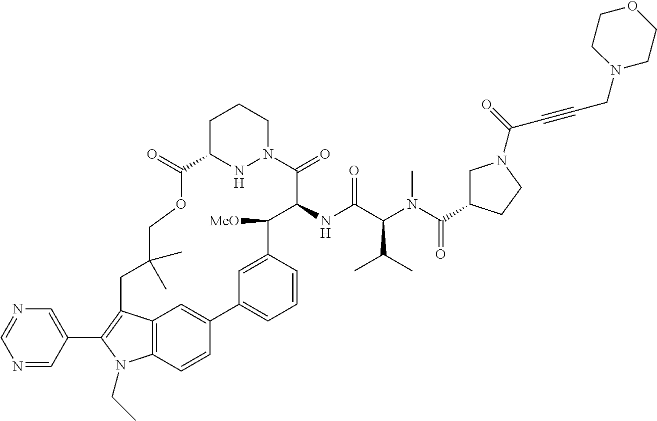 [SMILES: CCn1c(-c2cncnc2)c2c3cc(ccc31)-c1cccc(c1)[C@@H](OC)[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BB 70 | 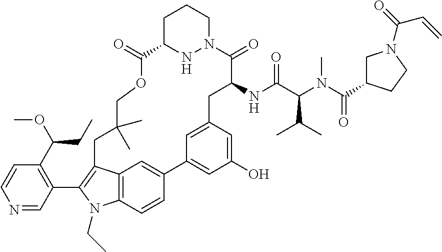 [SMILES: C=CC(=O)N1CC[C@H](C(=O)N(C)[C@H](C(=O)N[C@H]2Cc3cc(O)cc(c3)-c3ccc4c(c3)c(c(-c3cnccc3[C@H](CC)OC)n4CC)CC(C)(C)COC(=O)[C@@H]3CCCN(N3)C2=O)C(C)C)C1] |
| BB 73 | 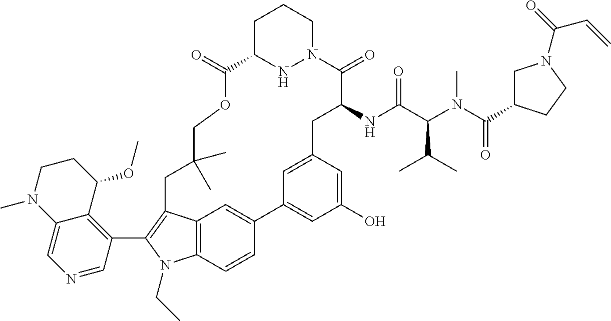 [SMILES: C=CC(=O)N1CC[C@H](C(=O)N(C)[C@H](C(=O)N[C@H]2Cc3cc(O)cc(c3)-c3ccc4c(c3)c(c(-c3cncc5c3[C@@H](OC)CCN5C)n4CC)CC(C)(C)COC(=O)[C@@H]3CCCN(N3)C2=O)C(C)C)C1] |
| BB 74 | 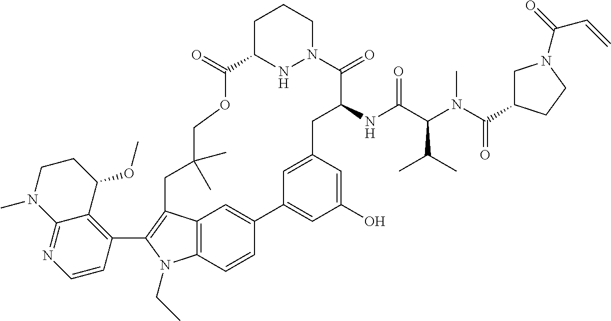 [SMILES: C=CC(=O)N1CC[C@H](C(=O)N(C)[C@H](C(=O)N[C@H]2Cc3cc(O)cc(c3)-c3ccc4c(c3)c(c(-c3ccnc5c3[C@@H](OC)CCN5C)n4CC)CC(C)(C)COC(=O)[C@@H]3CCCN(N3)C2=O)C(C)C)C1] |
| BB 75 | 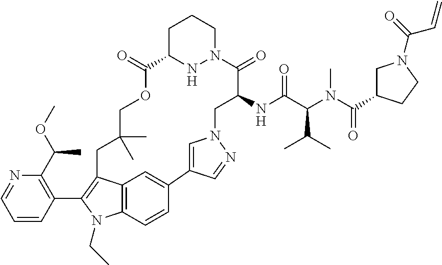 [SMILES: C=CC(=O)N1CC[C@H](C(=O)N(C)[C@H](C(=O)N[C@H]2Cn3cc(cn3)-c3ccc4c(c3)c(c(-c3cccnc3[C@H](C)OC)n4CC)CC(C)(C)COC(=O)[C@@H]3CCCN(N3)C2=O)C(C)C)C1] |
| BB 76 | 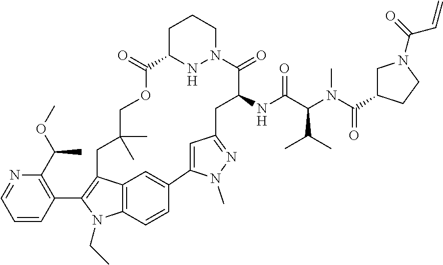 [SMILES: C=CC(=O)N1CC[C@H](C(=O)N(C)[C@H](C(=O)N[C@H]2Cc3cc(n(C)n3)-c3ccc4c(c3)c(c(-c3cccnc3[C@H](C)OC)n4CC)CC(C)(C)COC(=O)[C@@H]3CCCN(N3)C2=O)C(C)C)C1] |
| BB 77 | 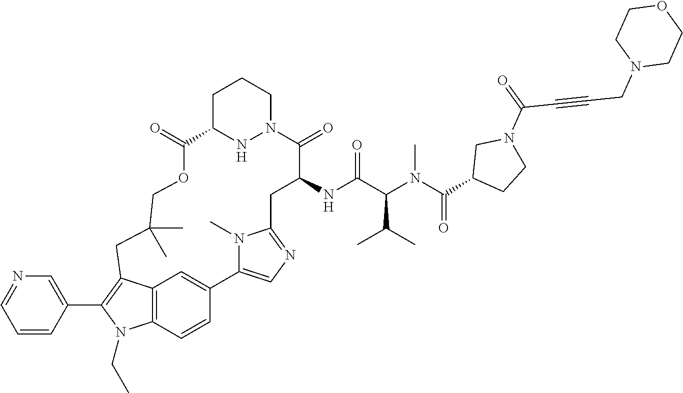 [SMILES: CCn1c(-c2cccnc2)c2c3cc(ccc31)-c1cnc(n1C)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BB 81 | 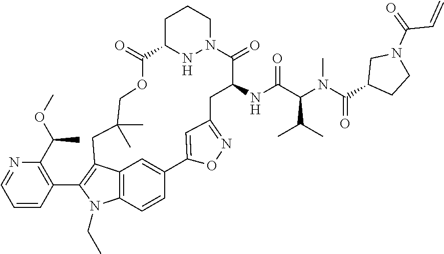 [SMILES: C=CC(=O)N1CC[C@H](C(=O)N(C)[C@H](C(=O)N[C@H]2Cc3cc(on3)-c3ccc4c(c3)c(c(-c3cccnc3[C@H](C)OC)n4CC)CC(C)(C)COC(=O)[C@@H]3CCCN(N3)C2=O)C(C)C)C1] |
| BB 83 | 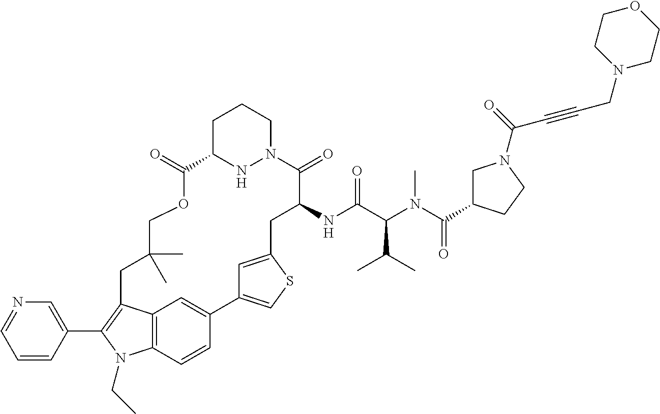 [SMILES: CCn1c(-c2cccnc2)c2c3cc(ccc31)-c1csc(c1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BB 85 | 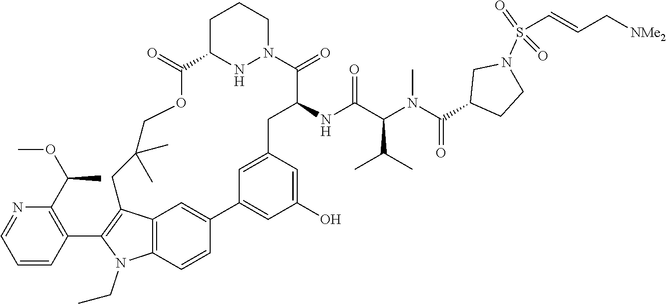 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(S(=O)(=O)/C=C/CN(C)C)C1)C(=O)N1CCC[C@H](N1)C(=O)OCC(C)(C)C2] |
| BB 86 | 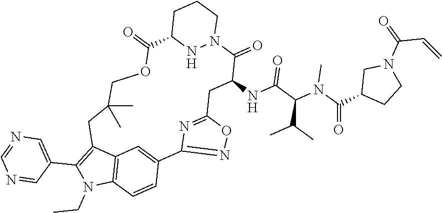 [SMILES: C=CC(=O)N1CC[C@H](C(=O)N(C)[C@H](C(=O)N[C@H]2Cc3nc(no3)-c3ccc4c(c3)c(c(-c3cncnc3)n4CC)CC(C)(C)COC(=O)[C@@H]3CCCN(N3)C2=O)C(C)C)C1] |
| BB 87 | 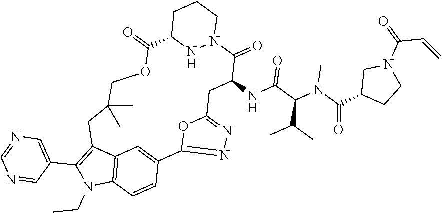 [SMILES: C=CC(=O)N1CC[C@H](C(=O)N(C)[C@H](C(=O)N[C@H]2Cc3nnc(o3)-c3ccc4c(c3)c(c(-c3cncnc3)n4CC)CC(C)(C)COC(=O)[C@@H]3CCCN(N3)C2=O)C(C)C)C1] |
| BB 88 | 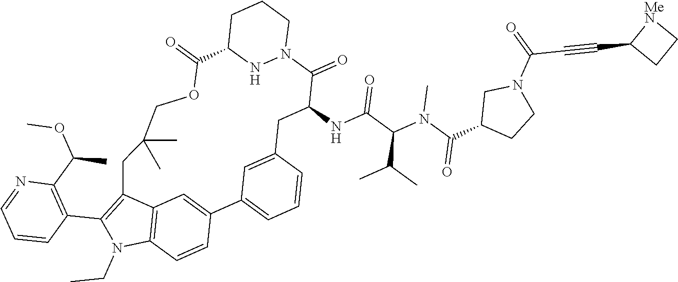 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)C#C[C@@H]3CCN3C)C1)C(=O)N1CCC[C@H](N1)C(=O)OCC(C)(C)C2] |
| BB 89 | 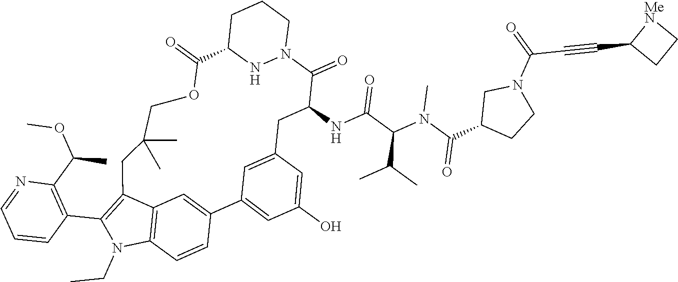 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)C#C[C@@H]3CCN3C)C1)C(=O)N1CCC[C@H](N1)C(=O)OCC(C)(C)C2] |
| BB 90 | 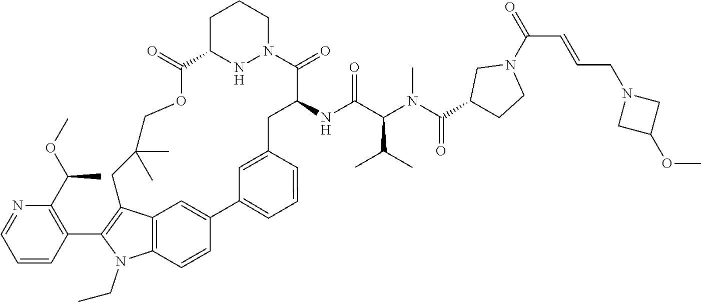 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)/C=C/CN3CC(OC)C3)C1)C(=O)N1CCC[C@H](N1)C(=O)OCC(C)(C)C2] |
| BB 91 | 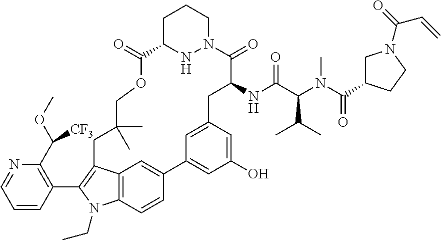 [SMILES: C=CC(=O)N1CC[C@H](C(=O)N(C)[C@H](C(=O)N[C@H]2Cc3cc(O)cc(c3)-c3ccc4c(c3)c(c(-c3cccnc3[C@@H](OC)C(F)(F)F)n4CC)CC(C)(C)COC(=O)[C@@H]3CCCN(N3)C2=O)C(C)C)C1] |
| BB 96 | 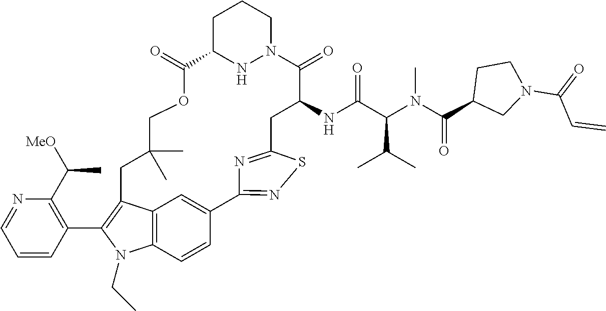 [SMILES: C=CC(=O)N1CC[C@H](C(=O)N(C)[C@H](C(=O)N[C@H]2Cc3nc(ns3)-c3ccc4c(c3)c(c(-c3cccnc3[C@H](C)OC)n4CC)CC(C)(C)COC(=O)[C@@H]3CCCN(N3)C2=O)C(C)C)C1] |
| BB 97 | 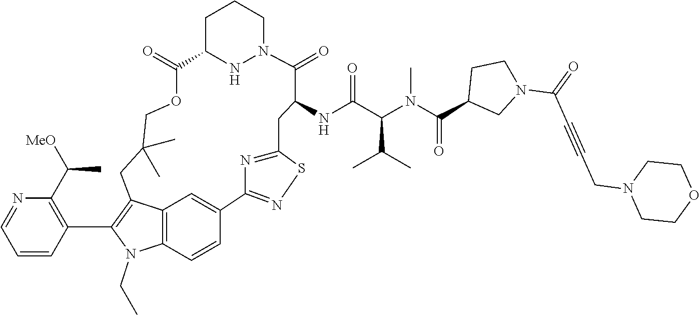 |
| BB 102 | 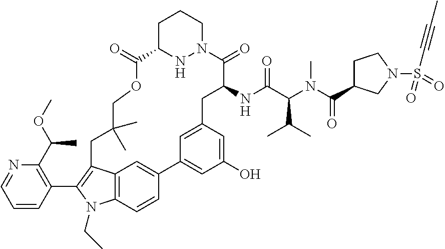 [SMILES: CC#CS(=O)(=O)N1CC[C@H](C(=O)N(C)[C@H](C(=O)N[C@H]2Cc3cc(O)cc(c3)-c3ccc4c(c3)c(c(-c3cccnc3[C@H](C)OC)n4CC)CC(C)(C)COC(=O)[C@@H]3CCCN(N3)C2=O)C(C)C)C1] |
| BB 103 | 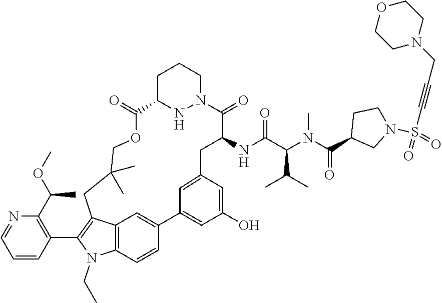 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(S(=O)(=O)C#CCN3CCOCC3)C1)C(=O)N1CCC[C@H](N1)C(=O)OCC(C)(C)C2] |
| BB 104 | 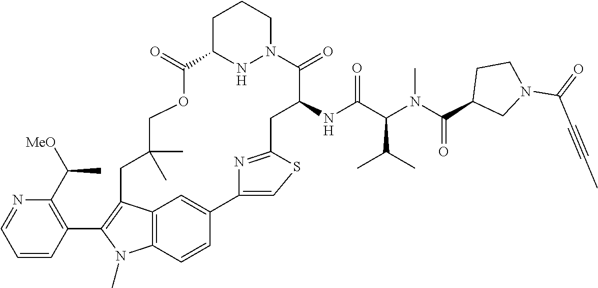 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4C)CC(C)(C)COC(=O)[C@@H]3CCCN(N3)C2=O)C(C)C)C1] |
| BB 106 | 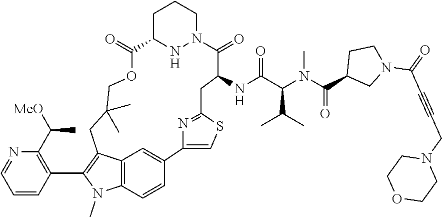 |
| BB 107 | 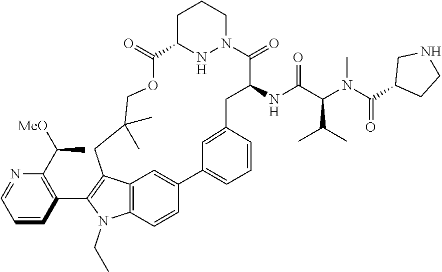 |
| BB 109 | 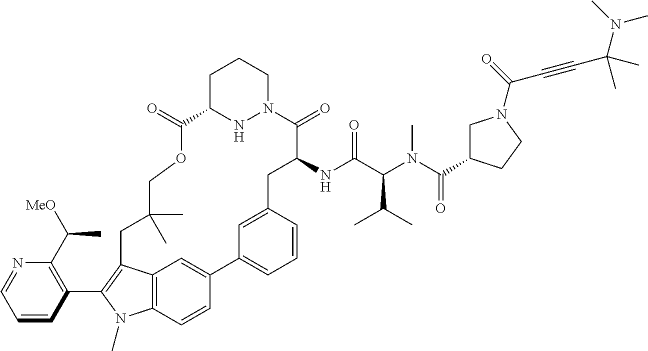 |
| BB 111 | 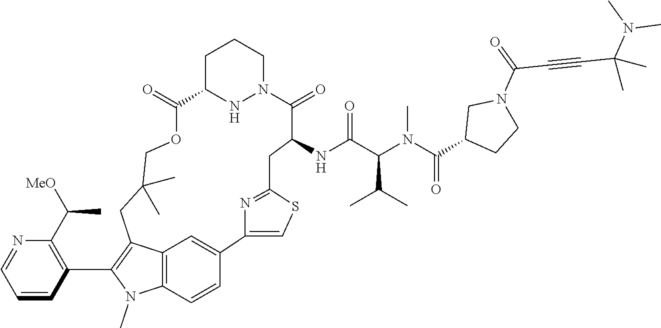 |
| BB 112 | 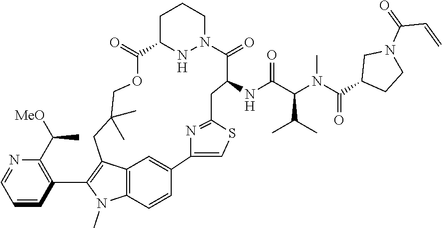 |
| BB 113 | 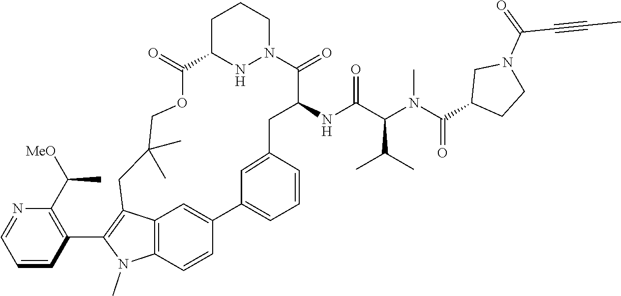 |
| BB 115 | 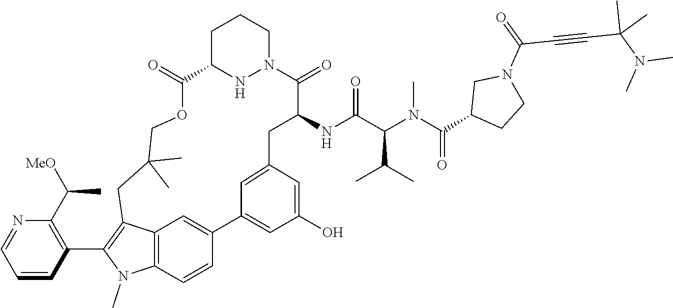 |
| BB 116 | 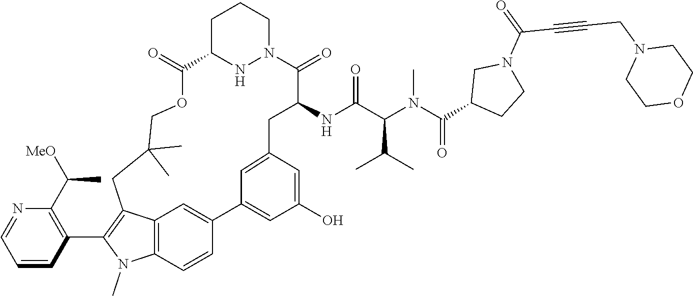 |
| BB 117 | 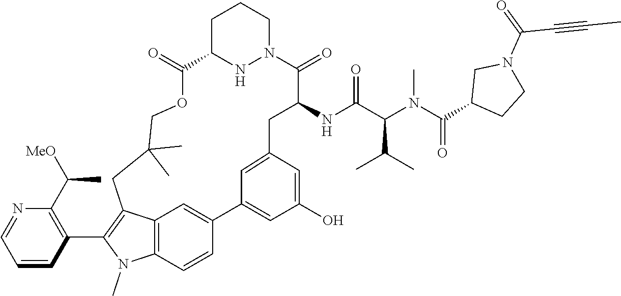 |
| BB 118 | 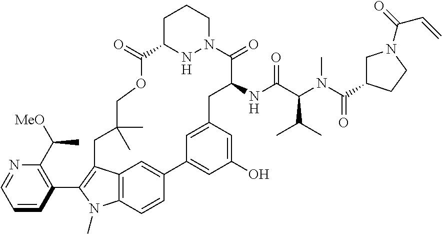 |
| BB 119 | 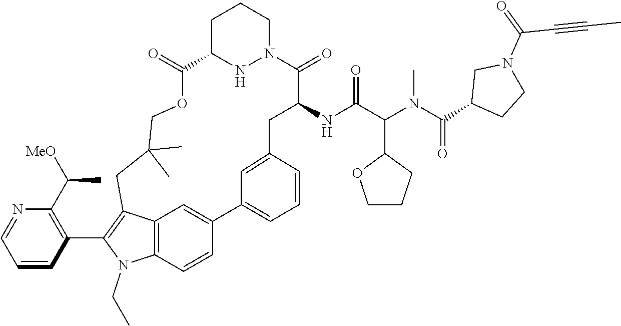 |
| BB 120 | 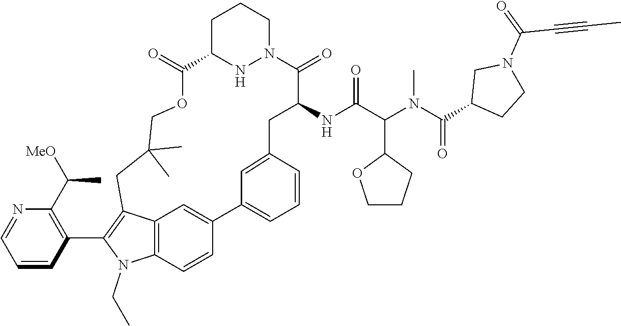 |
| BB 121 | 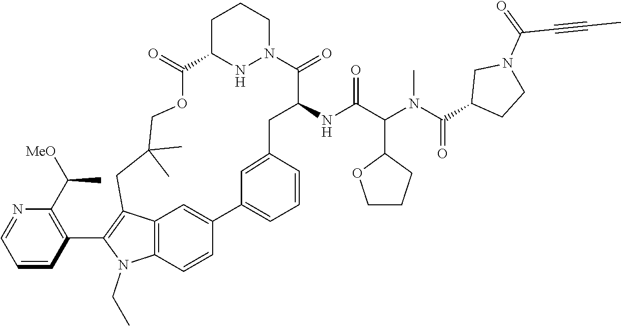 |
| BB 122 | 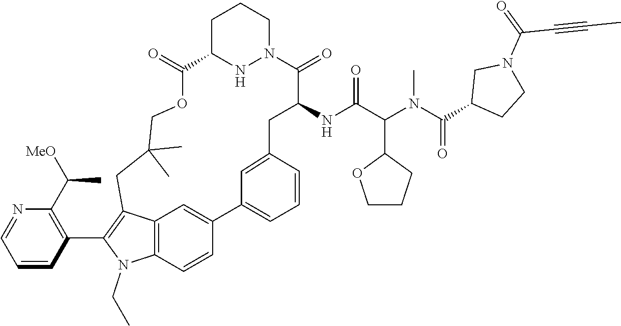 |
| BB 123 | 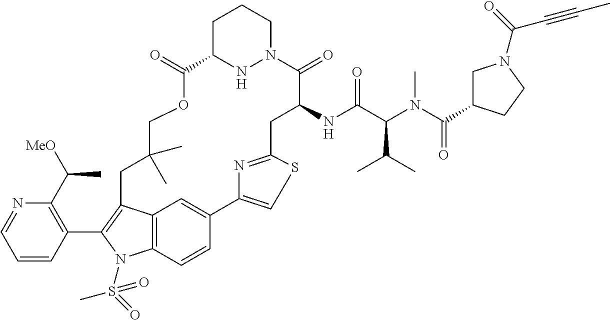 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4S(C)(=O)=O)CC(C)(C)COC(=O)[C@@H]3CCCN(N3)C2=O)C(C)C)C1] |
| BB 124 | 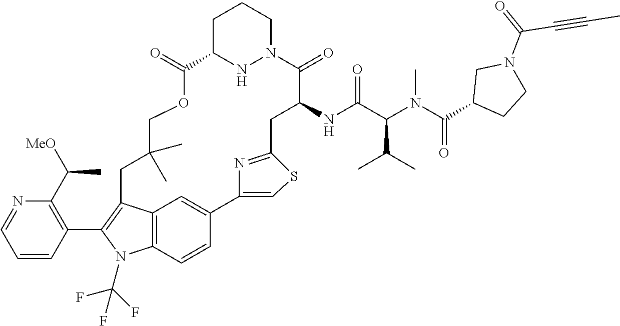 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4C(F)(F)F)CC(C)(C)COC(=O)[C@@H]3CCCN(N3)C2=O)C(C)C)C1] |
| BB 126 | 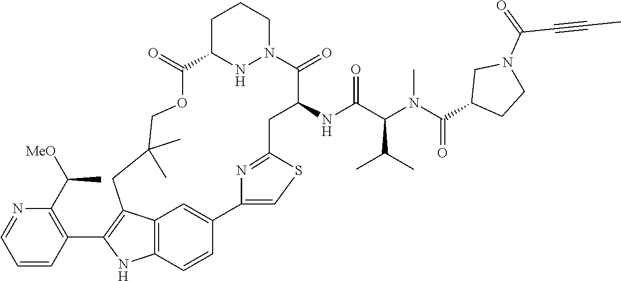 [SMILES: CC#CC(=O)N1CC[C@H](C(=O)N(C)[C@H](C(=O)N[C@H]2Cc3nc(cs3)-c3ccc4[nH]c(-c5cccnc5[C@H](C)OC)c(c4c3)CC(C)(C)COC(=O)[C@@H]3CCCN(N3)C2=O)C(C)C)C1] |
| BB 127 | 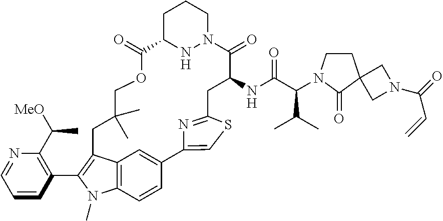 |
| BB 128 | 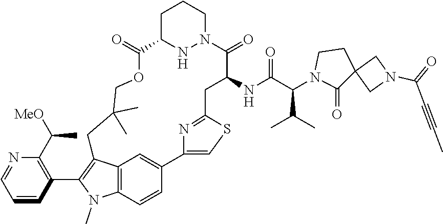 |
| BB 129 | 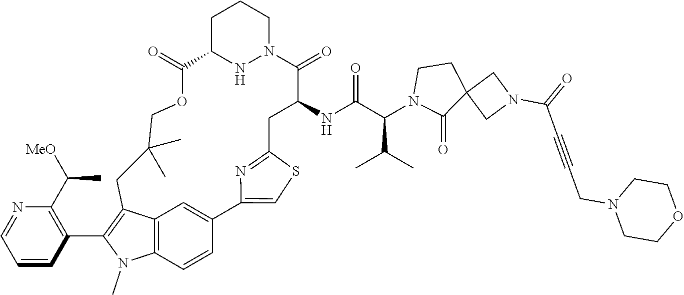 |
| BB 130 | 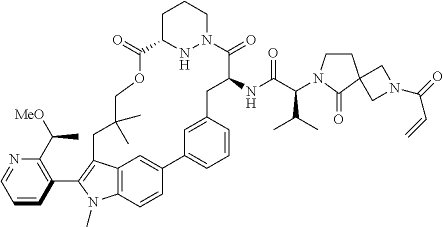 |
| BB 131 | 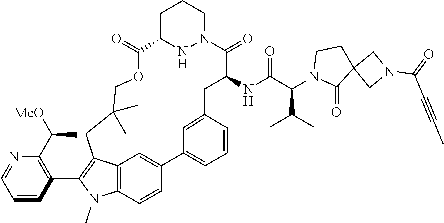 |
| BB 132 | 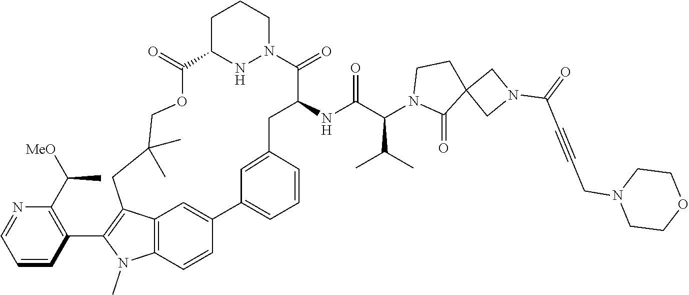 |
| BB 139 | 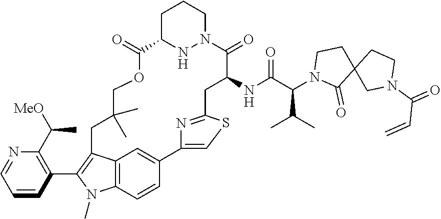 |
| BB 140 | 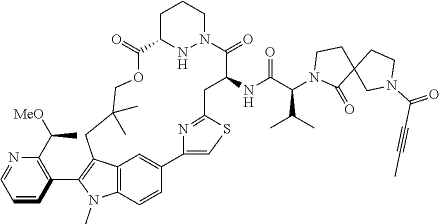 |
| BB 141 | 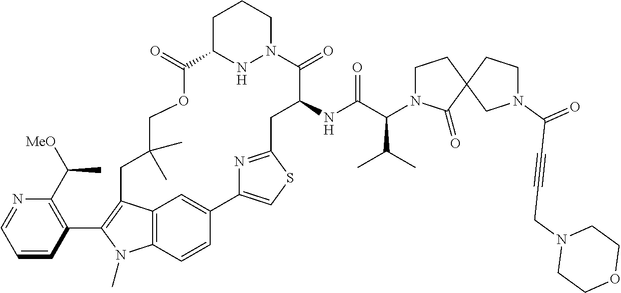 |
| BB 142 | 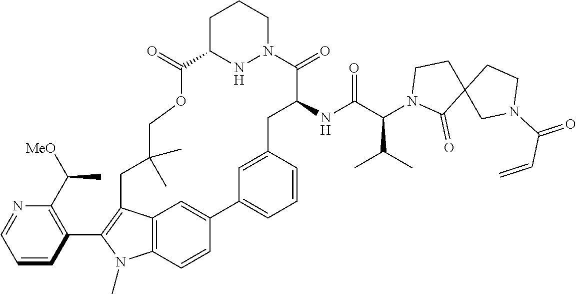 |
| BB 143 | 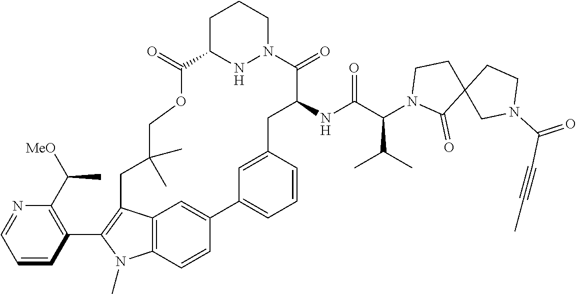 |
| BB 144 | 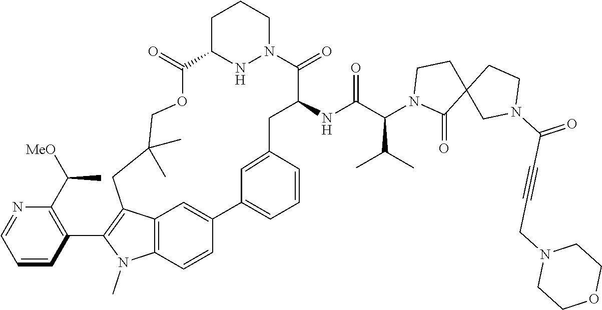 |
| BB 145 | 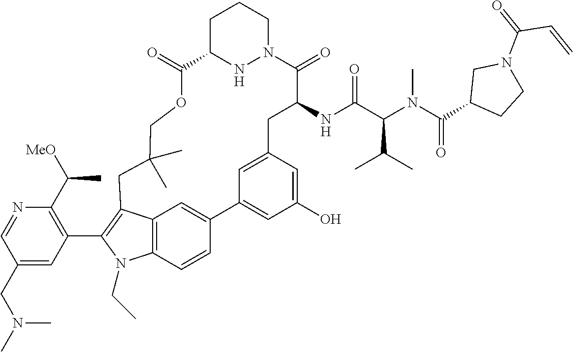 [SMILES: C=CC(=O)N1CC[C@H](C(=O)N(C)[C@H](C(=O)N[C@H]2Cc3cc(O)cc(c3)-c3ccc4c(c3)c(c(-c3cc(CN(C)C)cnc3[C@H](C)OC)n4CC)CC(C)(C)COC(=O)[C@@H]3CCCN(N3)C2=O)C(C)C)C1] |
| BB 146 | 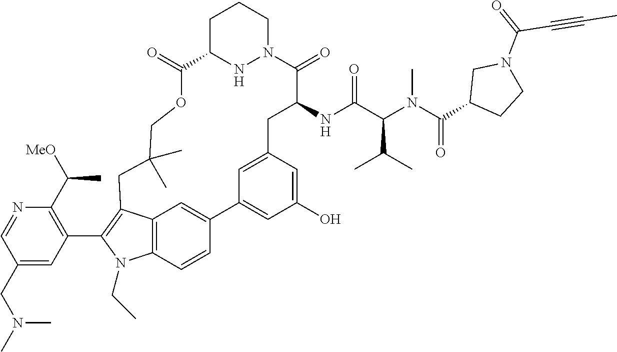 [SMILES: CC#CC(=O)N1CC[C@H](C(=O)N(C)[C@H](C(=O)N[C@H]2Cc3cc(O)cc(c3)-c3ccc4c(c3)c(c(-c3cc(CN(C)C)cnc3[C@H](C)OC)n4CC)CC(C)(C)COC(=O)[C@@H]3CCCN(N3)C2=O)C(C)C)C1] |
| BB 147 | 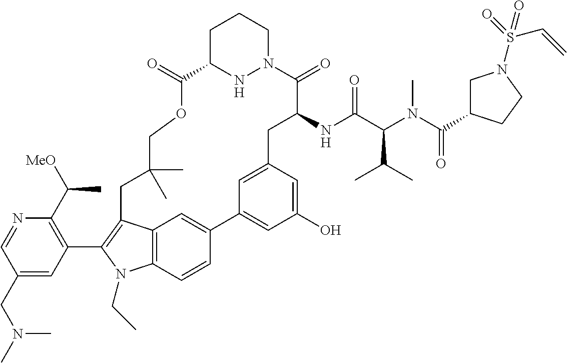 [SMILES: C=CS(=O)(=O)N1CC[C@H](C(=O)N(C)[C@H](C(=O)N[C@H]2Cc3cc(O)cc(c3)-c3ccc4c(c3)c(c(-c3cc(CN(C)C)cnc3[C@H](C)OC)n4CC)CC(C)(C)COC(=O)[C@@H]3CCCN(N3)C2=O)C(C)C)C1] |
| BB 148 | 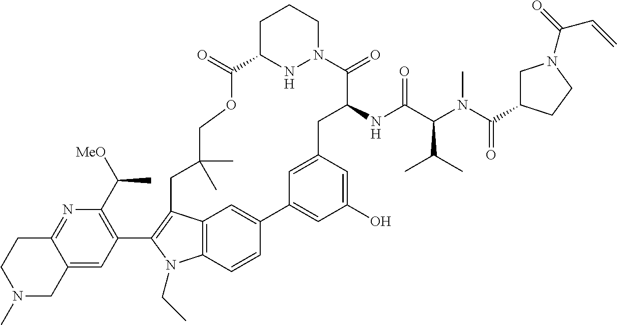 [SMILES: C=CC(=O)N1CC[C@H](C(=O)N(C)[C@H](C(=O)N[C@H]2Cc3cc(O)cc(c3)-c3ccc4c(c3)c(c(-c3cc5c(nc3[C@H](C)OC)CCN(C)C5)n4CC)CC(C)(C)COC(=O)[C@@H]3CCCN(N3)C2=O)C(C)C)C1] |
| BB 149 | 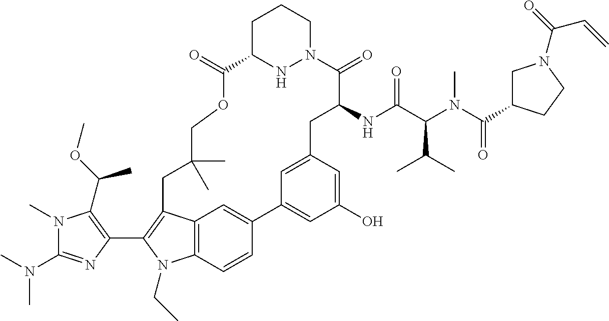 [SMILES: C=CC(=O)N1CC[C@H](C(=O)N(C)[C@H](C(=O)N[C@H]2Cc3cc(O)cc(c3)-c3ccc4c(c3)c(c(-c3nc(N(C)C)n(C)c3[C@H](C)OC)n4CC)CC(C)(C)COC(=O)[C@@H]3CCCN(N3)C2=O)C(C)C)C1] |
| BB 150 | 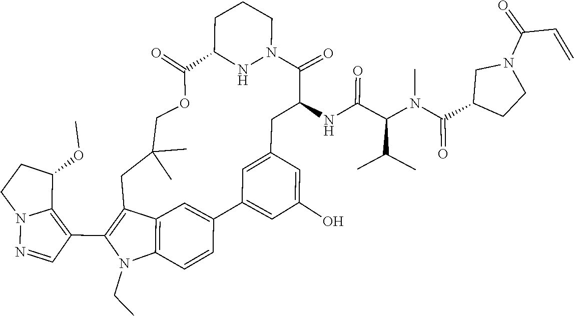 [SMILES: C=CC(=O)N1CC[C@H](C(=O)N(C)[C@H](C(=O)N[C@H]2Cc3cc(O)cc(c3)-c3ccc4c(c3)c(c(-c3cnn5c3[C@@H](OC)CC5)n4CC)CC(C)(C)COC(=O)[C@@H]3CCCN(N3)C2=O)C(C)C)C1] |
| BB 161 | 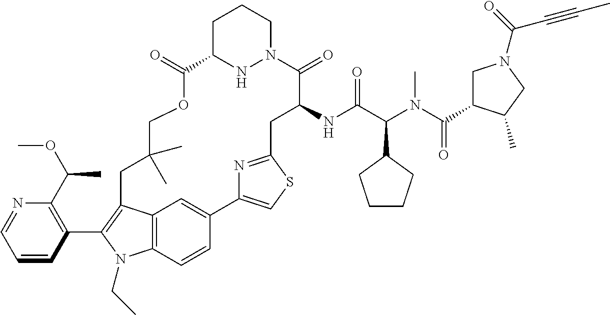 |
| BB 162 | 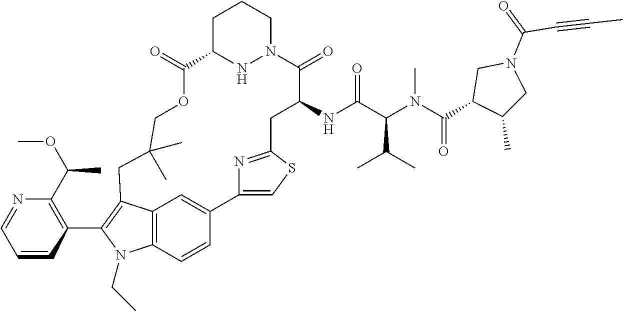 |
| BB 163 | 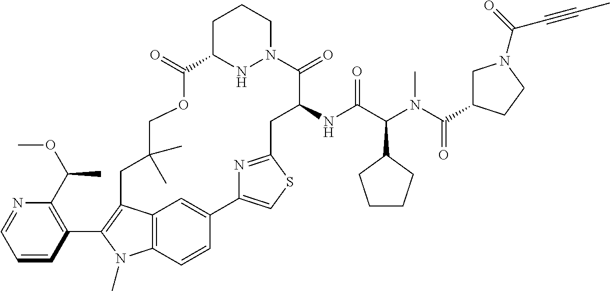 |
| BB 164 | 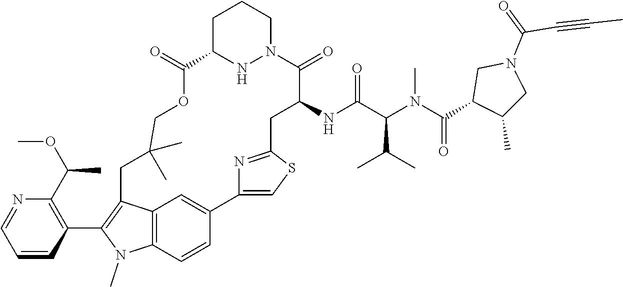 |
| BB 165 | 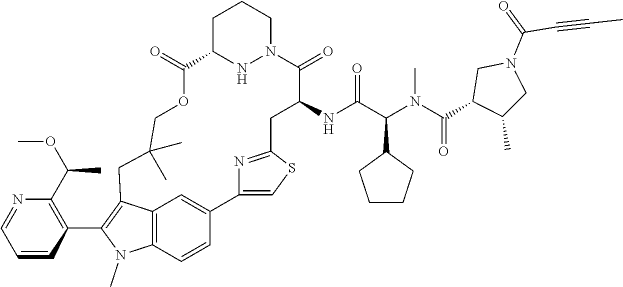 |
| BB 167 | 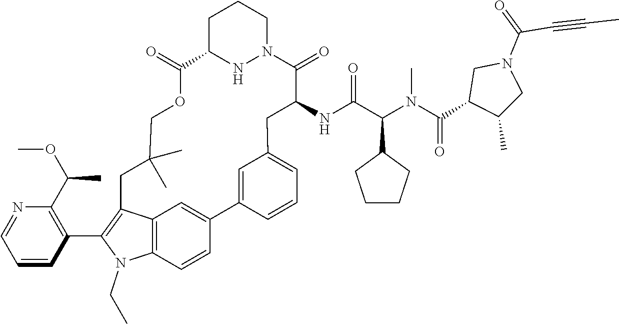 |
| BB 168 | 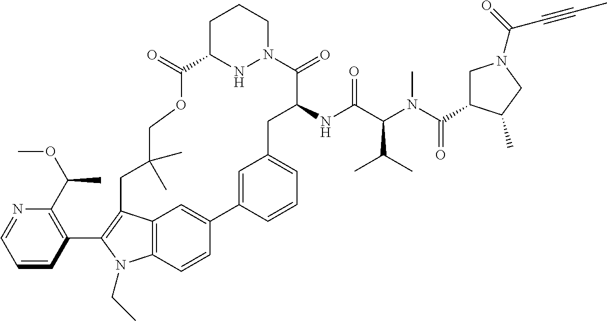 |
| BB 169 | 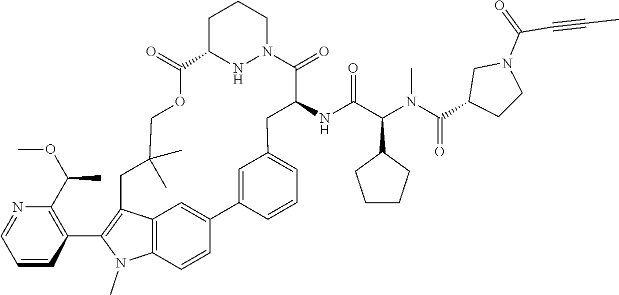 |
| BB 170 | 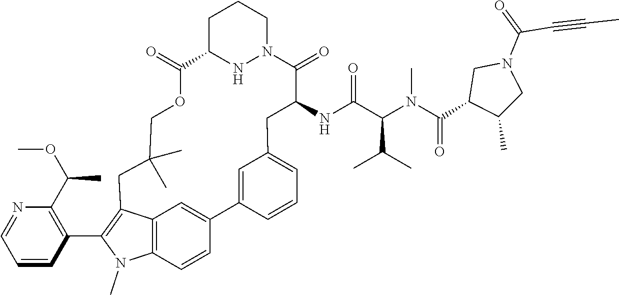 |
| BB 171 | 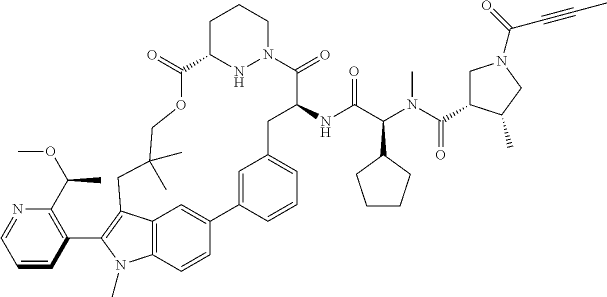 |
| BB 172 | 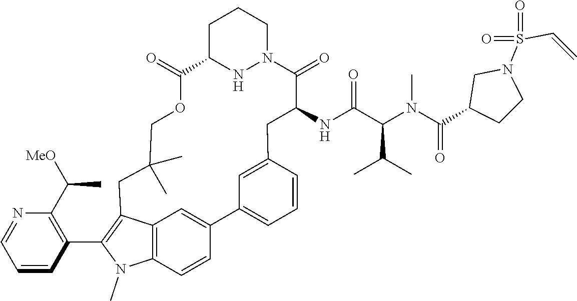 |
| BB 173 | 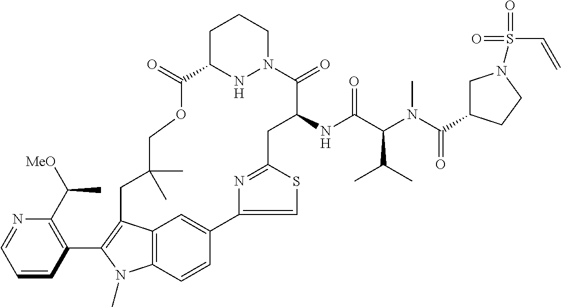 |
| BB 174 | 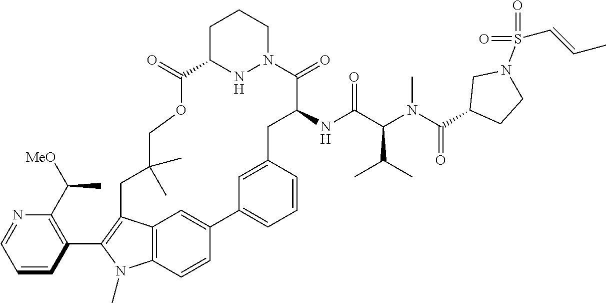 |
| BB 175 | 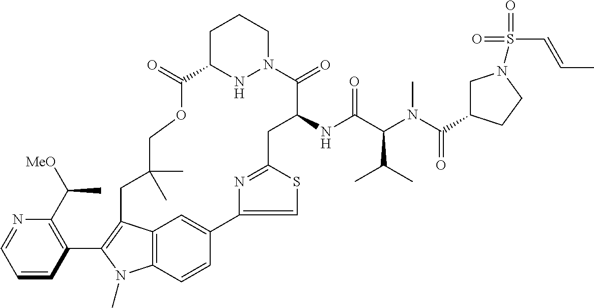 |
| BB 176 | 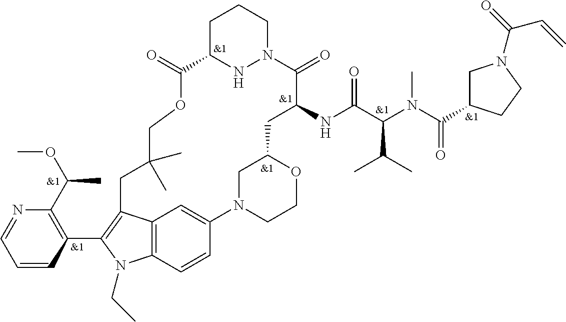 |
| BB 177 | 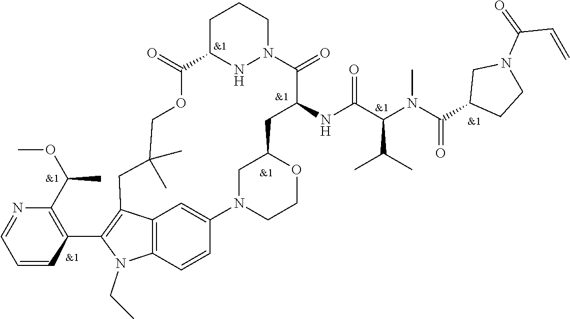 |
| BB 178 | 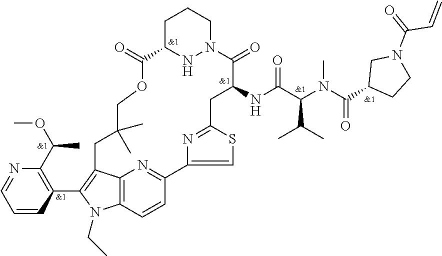 |
| BB 179 | 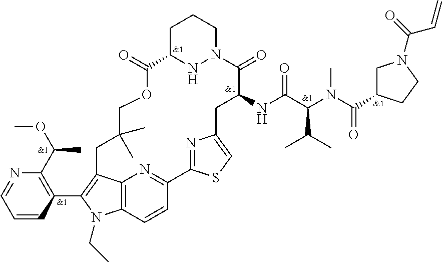 |
| BB 180 | 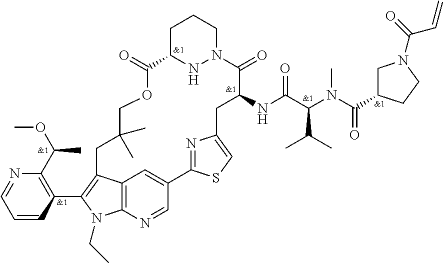 |
| BB 181 | 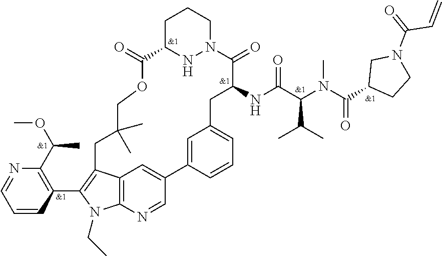 |
| BB 182 | 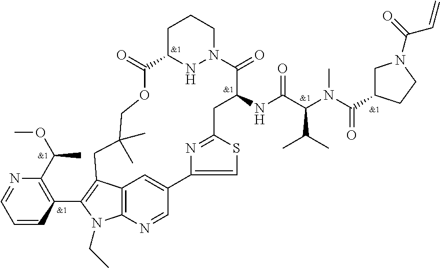 |
| BB 183 | 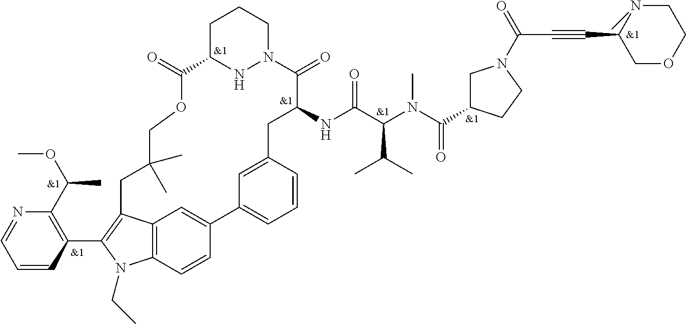 |
| BB 184 | 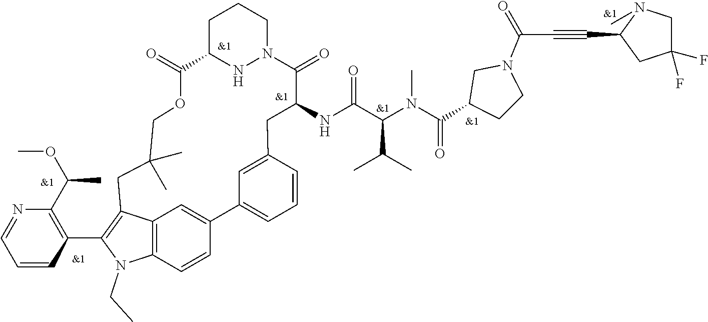 |
| BB 185 | 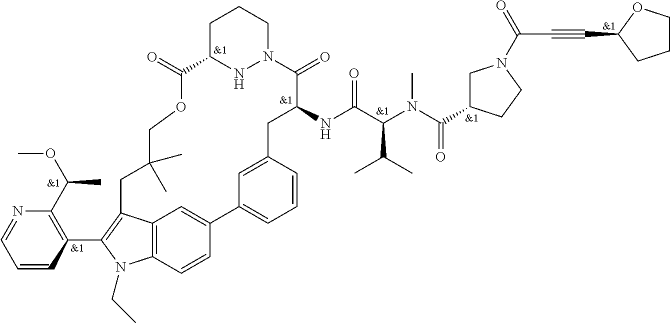 |
| BB 186 | 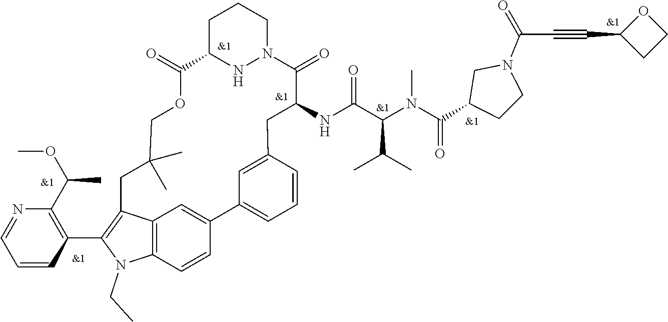 |
| BB 187 | 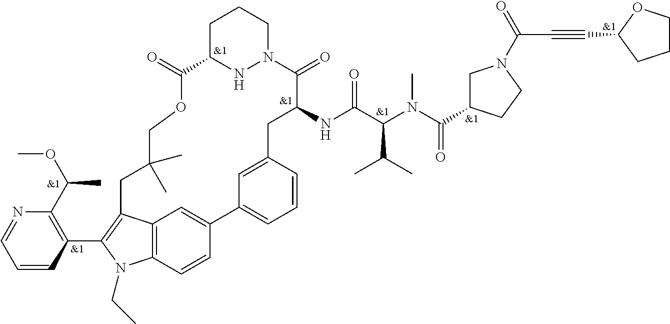 |
| BB 188 | 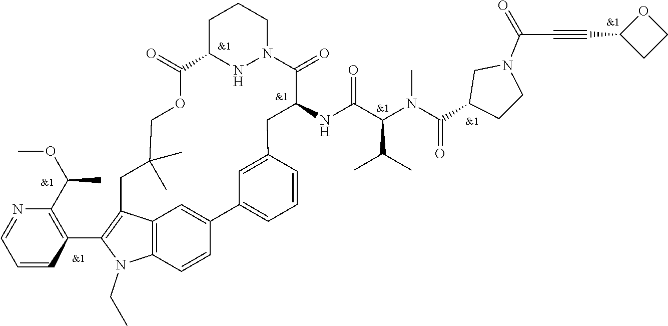 |
| BB 189 | 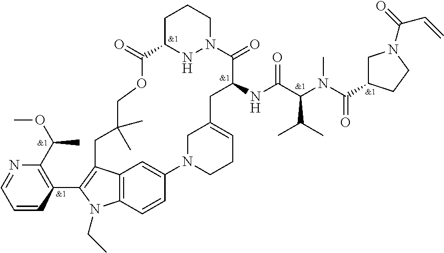 |
| BB 190 | 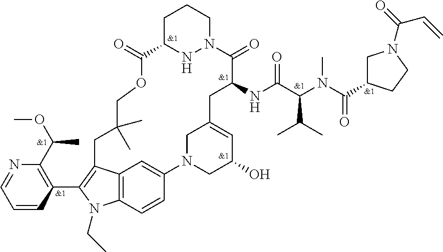 |
| BB 191 | 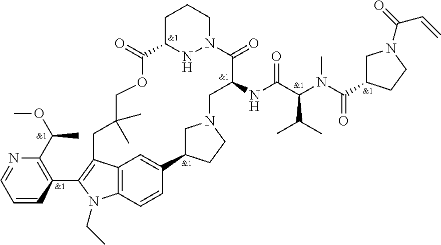 |
| BB 192 | 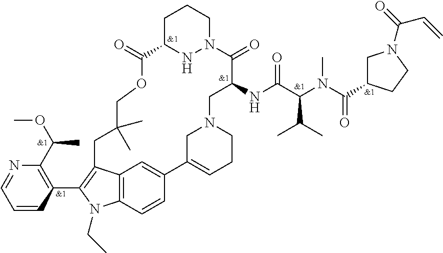 |
| BB 194 | 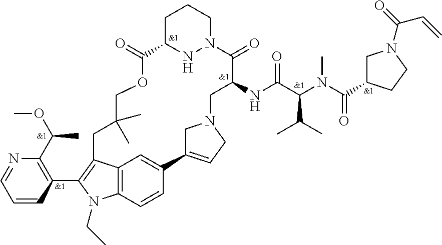 |
| BB 195 | 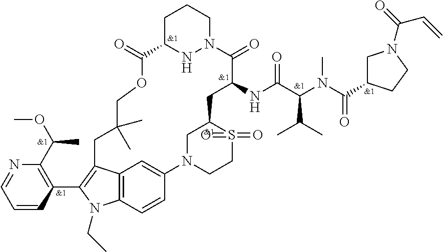 |
| BB 196 | 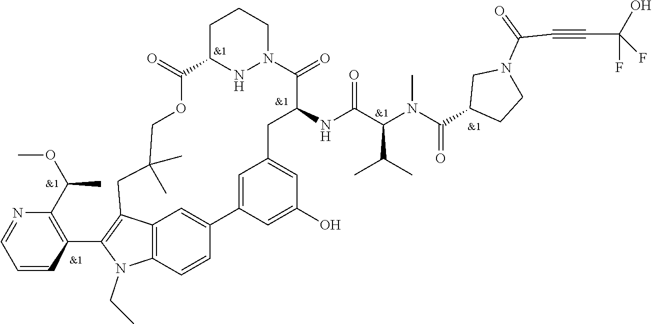 |
| BB 197 | 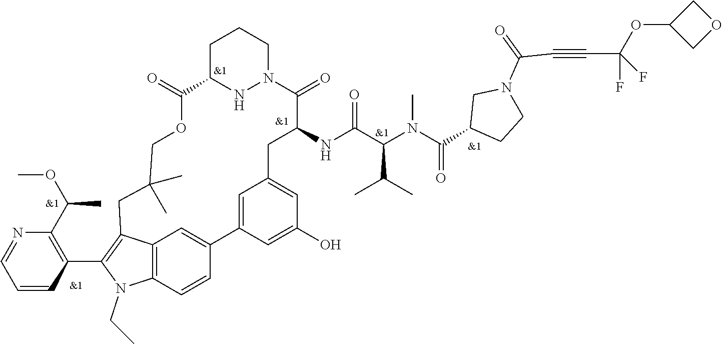 |
| BB 198 | 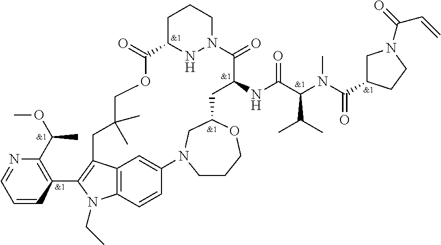 |
| BB 199 | 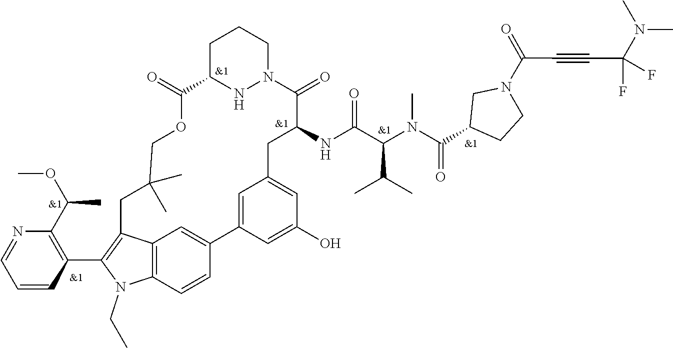 |
| BB 200 | 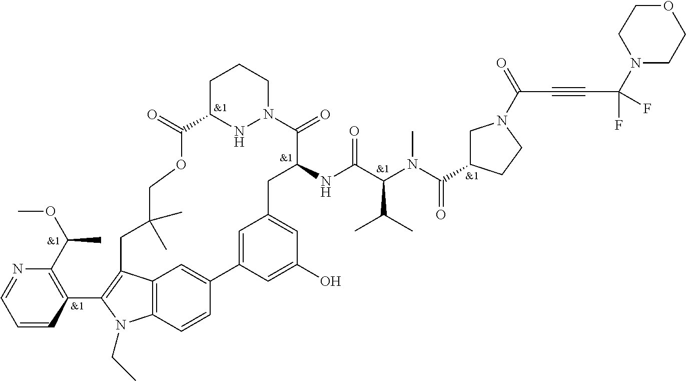 |
| BB 201 | 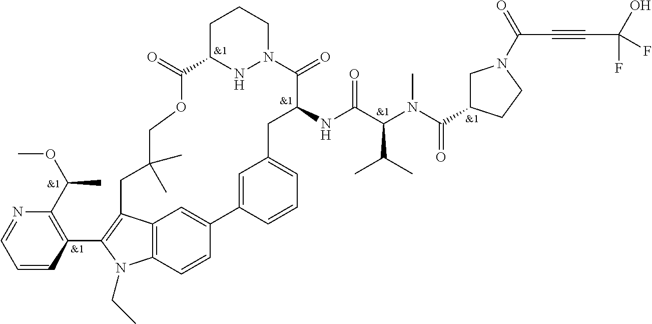 |
| BB 202 | 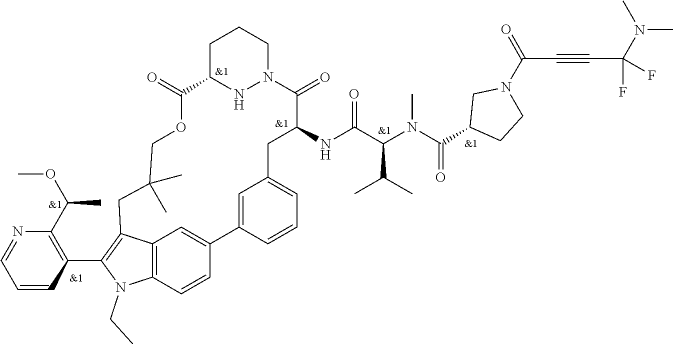 |
| BB 203 | 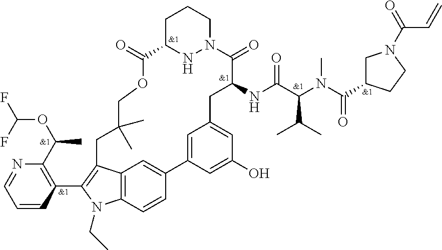 |
| BB 204 | 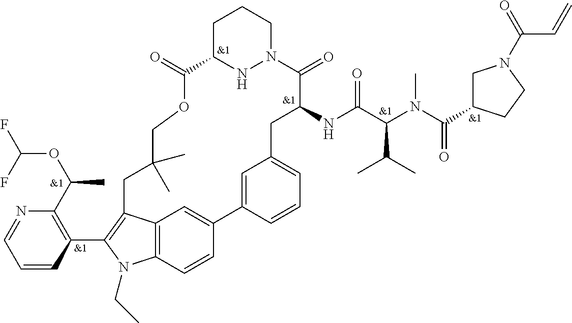 |
| BB 205 | 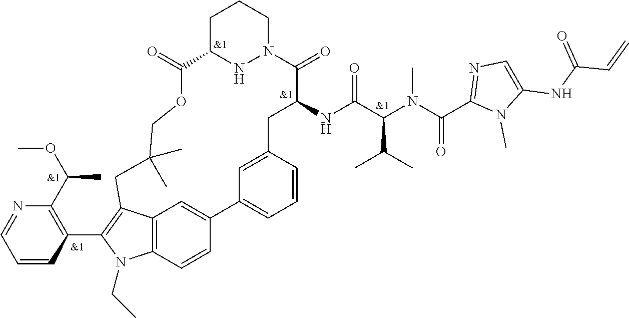 |
| BB 206 | 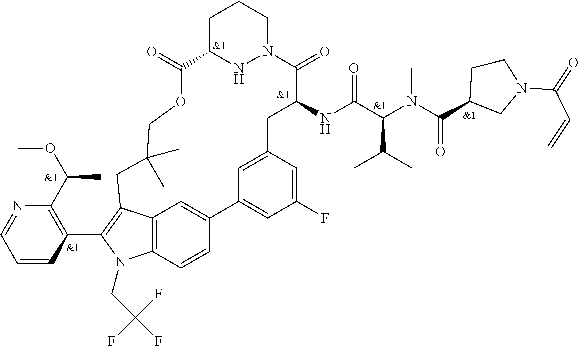 |
| BB 207 | 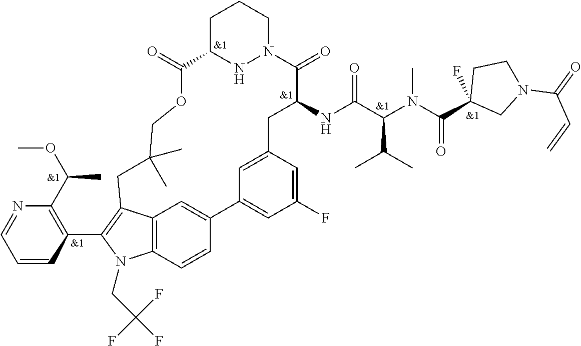 |
| BB 208 | 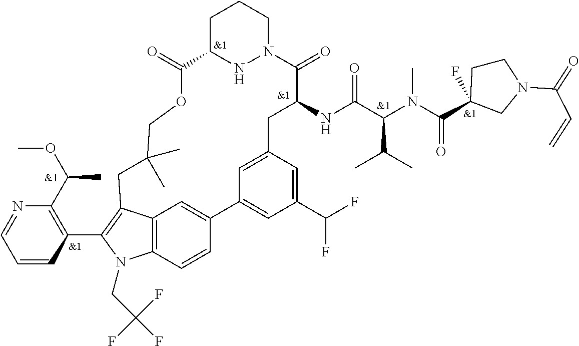 |
| BB 209 | 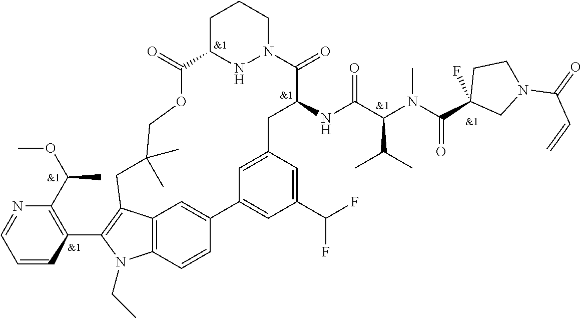 |
| BB 210 | 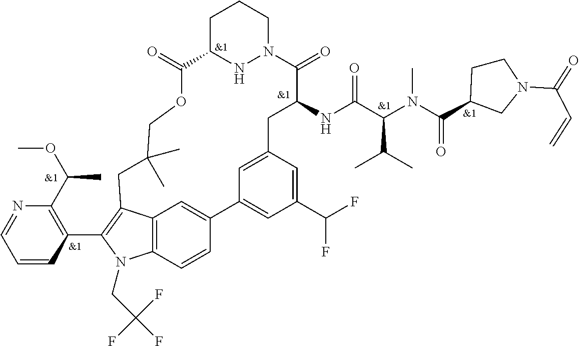 |
| BB 211 | 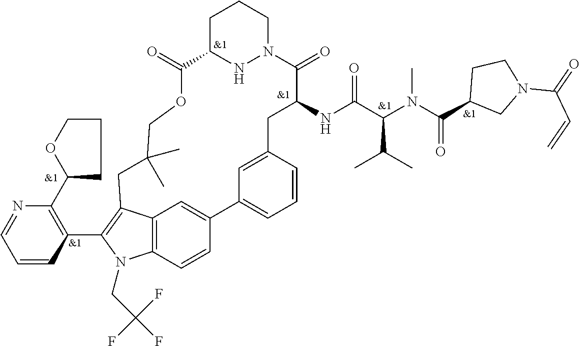 |
| BB 212 | 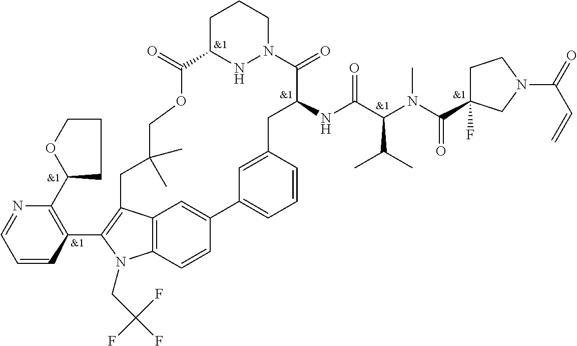 |
| BB 213 | 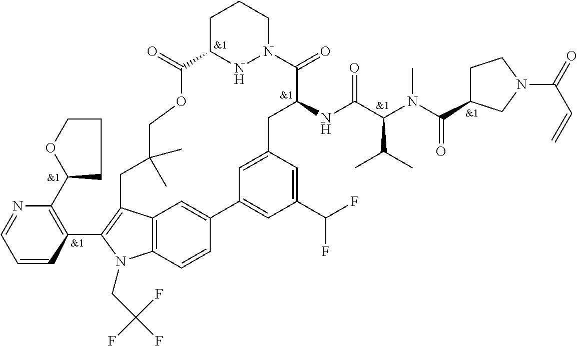 |
| BB 214 | 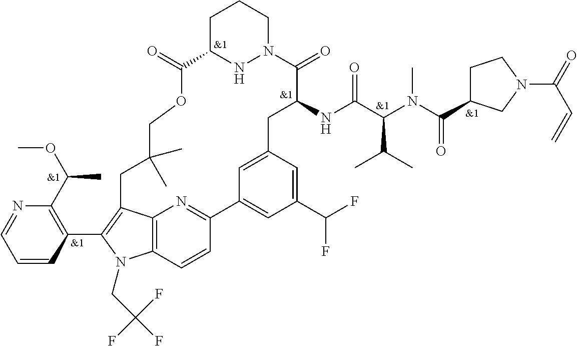 |
| BB 215 | 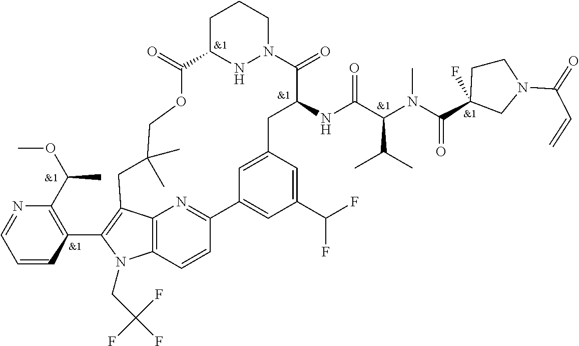 |
| BB 216 | 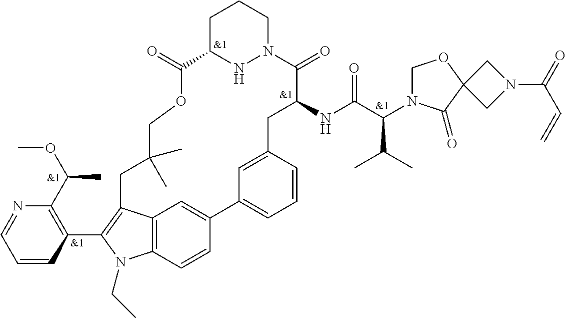 |
| BB 217 | 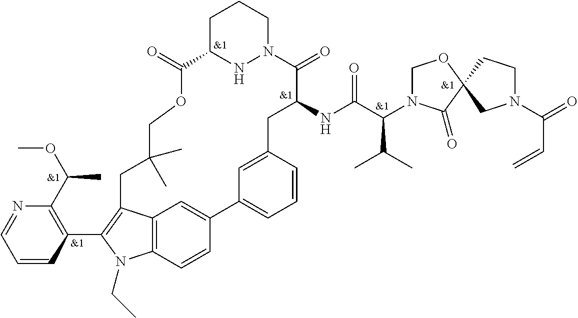 |
| BB 218 | 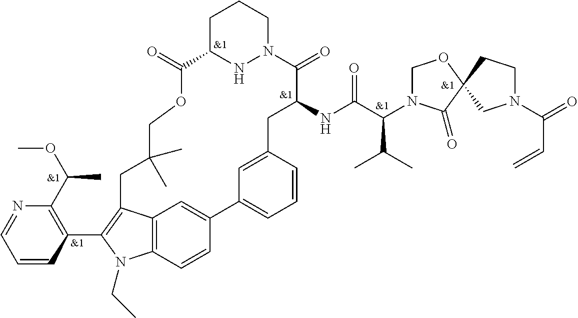 |
| BB 219 | 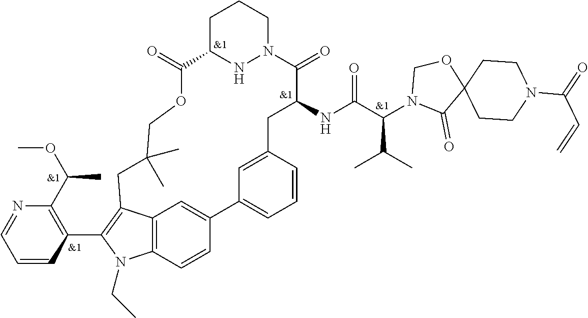 |
| BB 220 | 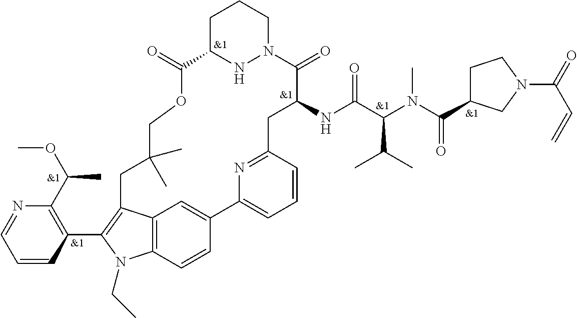 |
| BB 221 | 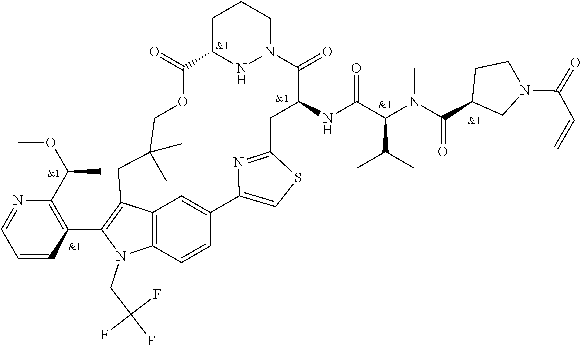 |
| BB 222 | 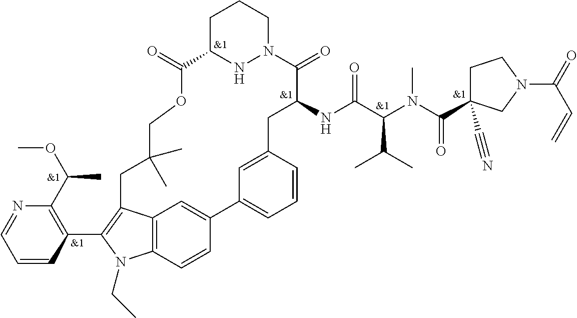 |
| BB 223 | 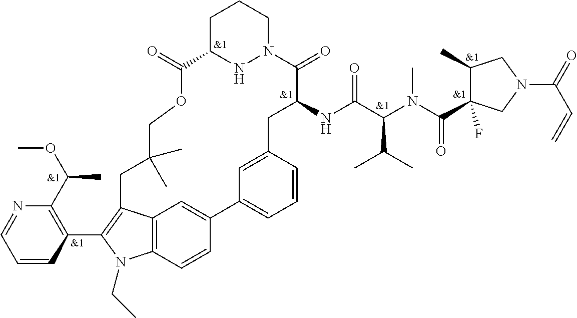 |
| BB 224 | 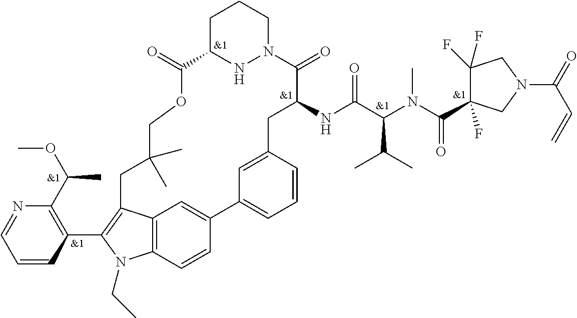 |
| BB 225 | 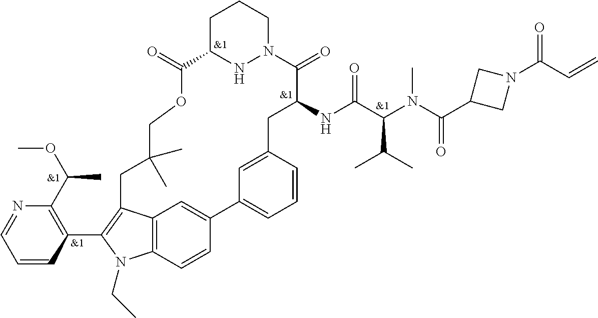 |
| BB 226 | 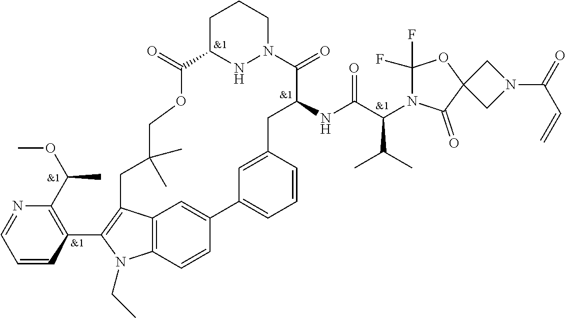 |
| BB 227 | 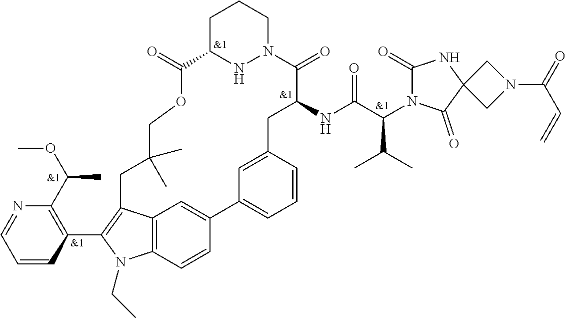 |
| BB 228 | 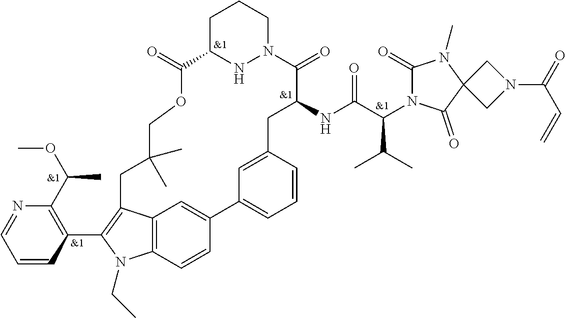 |
| BB 229 | 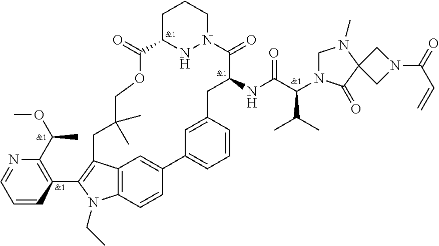 |
| BB 230 | 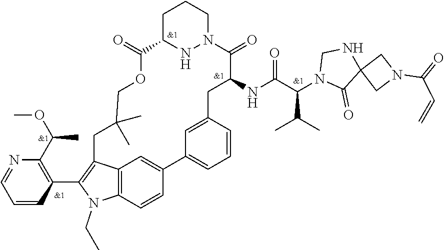 |
| BB 231 | 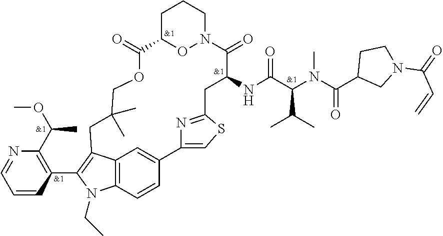 |
| BB 232 | 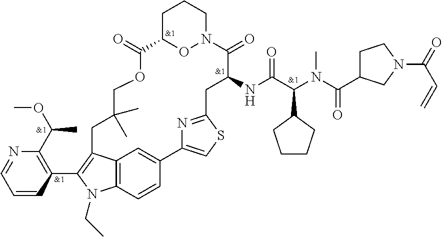 |
| BB 233 | 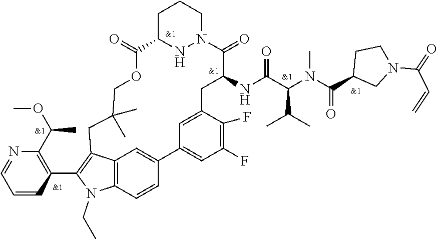 |
| BB 234 | 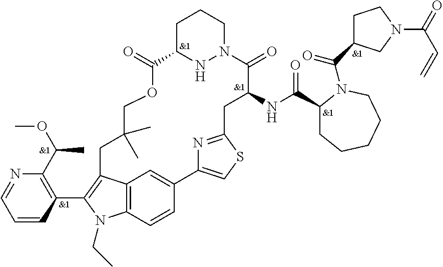 |
| BB 235 | 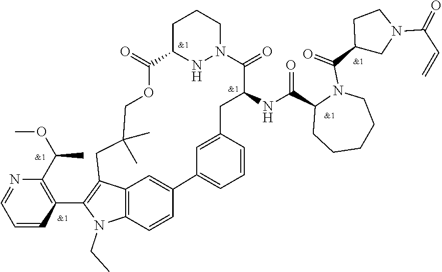 |
| BB 236 | 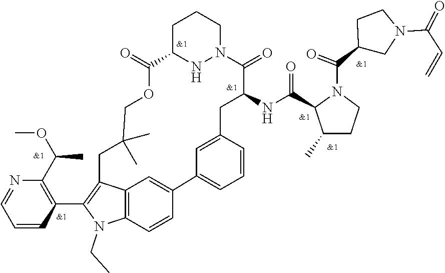 |
| BB 237 | 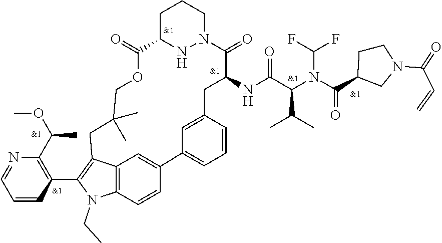 |
| BB 238 | 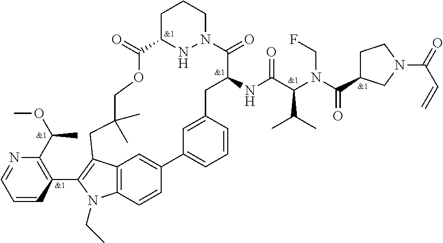 |
| BB 239 | 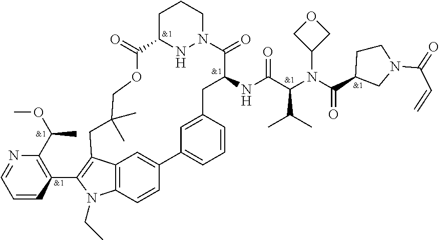 |
| BB 240 | 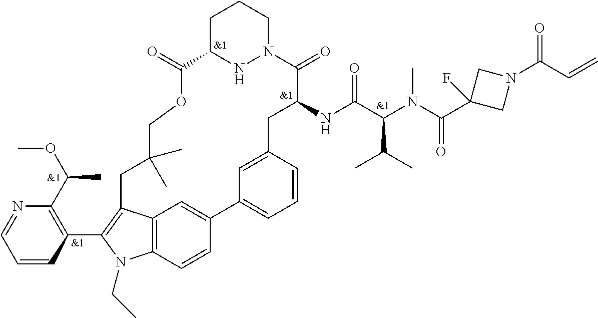 |
| BB 241 | 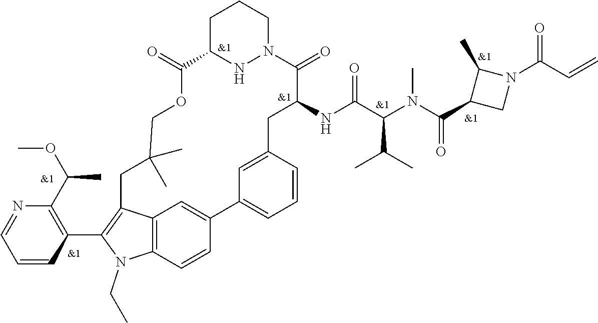 |
| BB 242 | 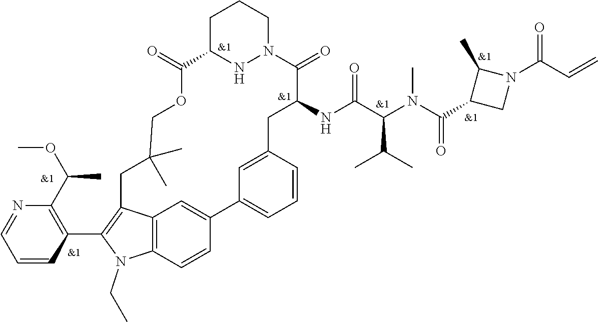 |
| BB 243 | 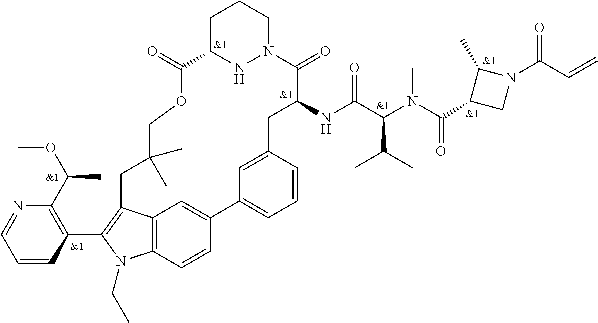 |
| BB 244 | 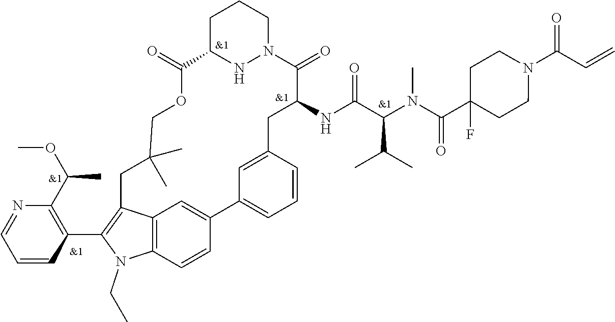 |
| BB 245 | 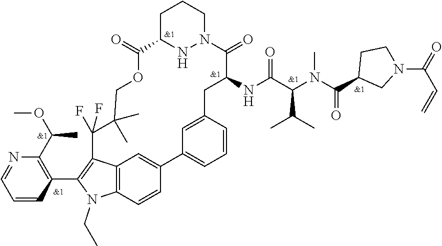 |
| BB 246 | 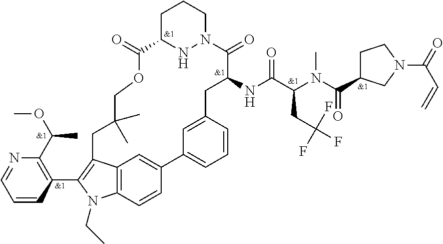 |
| BB 247 | 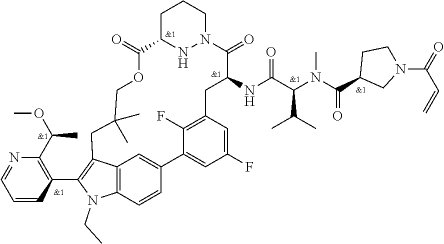 |
| BB 248 | 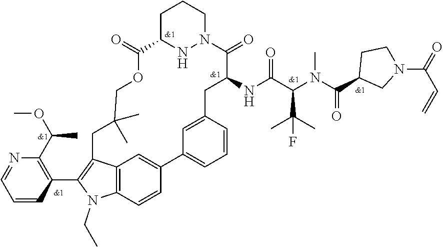 |
| BB 249 | 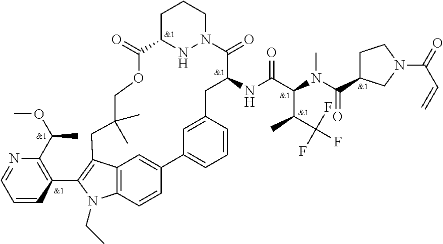 |
| BB 250 | 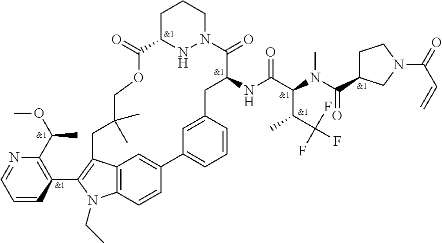 |
| BB 251 | 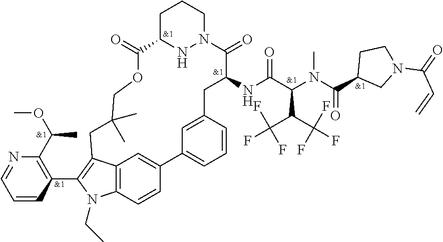 |
| BB 252 | 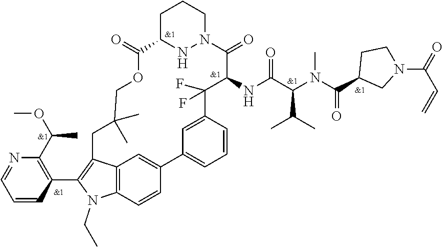 |
| BB 253 | 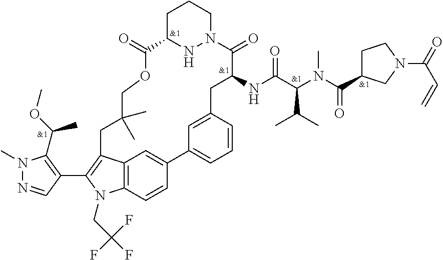 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)CC(C)(C)COC(=O)[C@@H]3CCCN(N3)C2=O)C(C)C)C1] |
| BB 254 | 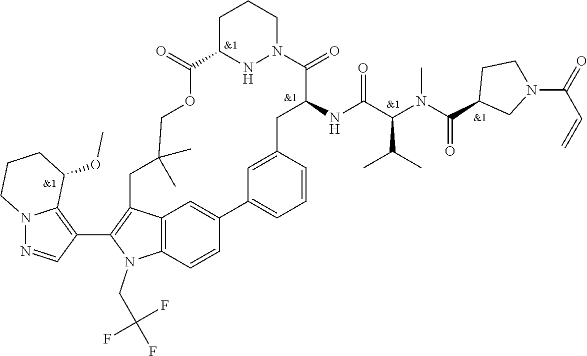 [SMILES: C=CC(=O)N1CC[C@H](C(=O)N(C)[C@H](C(=O)N[C@H]2Cc3cccc(c3)-c3ccc4c(c3)c(c(-c3cnn5c3[C@@H](OC)CCC5)n4CC(F)(F)F)CC(C)(C)COC(=O)[C@@H]3CCCN(N3)C2=O)C(C)C)C1] |
| BB 255 | 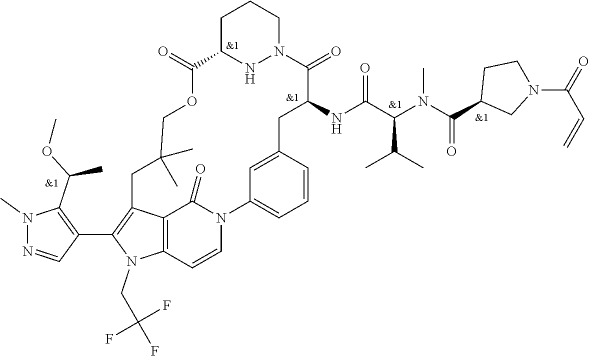 [SMILES: C=CC(=O)N1CC[C@H](C(=O)N(C)[C@H](C(=O)N[C@H]2Cc3cccc(c3)-n3ccc4c(c(c(-c5cnn(C)c5[C@H](C)OC)n4CC(F)(F)F)CC(C)(C)COC(=O)[C@@H]4CCCN(N4)C2=O)c3=O)C(C)C)C1] |
| BB 256 | 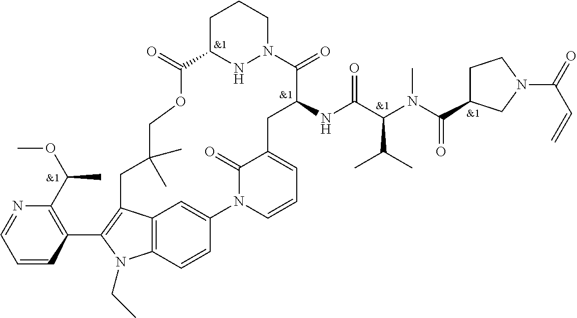 |
| BB 257 | 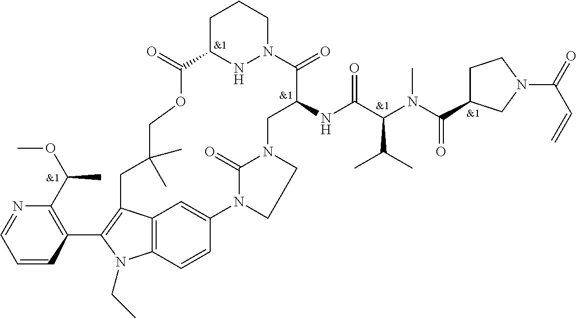 |
| BB 258 | 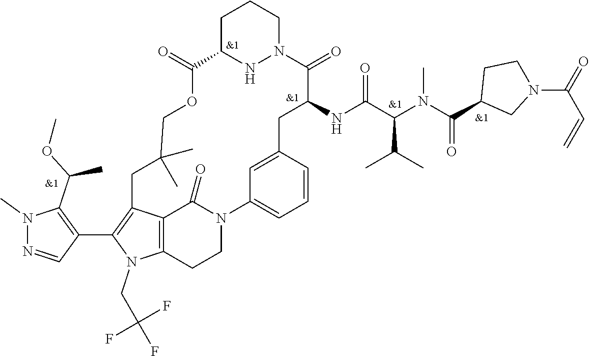 [SMILES: C=CC(=O)N1CC[C@H](C(=O)N(C)[C@H](C(=O)N[C@H]2Cc3cccc(c3)N3CCc4c(c(c(-c5cnn(C)c5[C@H](C)OC)n4CC(F)(F)F)CC(C)(C)COC(=O)[C@@H]4CCCN(N4)C2=O)C3=O)C(C)C)C1] |
| BB 259 | 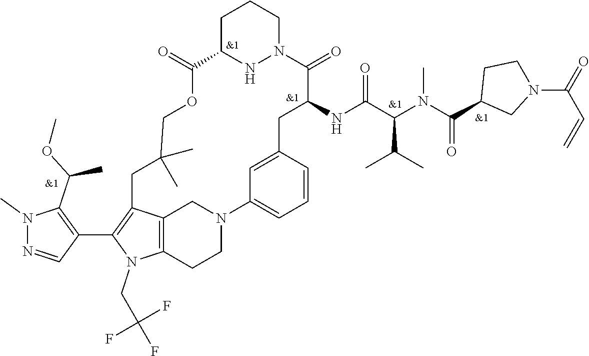 [SMILES: C=CC(=O)N1CC[C@H](C(=O)N(C)[C@H](C(=O)N[C@H]2Cc3cccc(c3)N3CCc4c(c(c(-c5cnn(C)c5[C@H](C)OC)n4CC(F)(F)F)CC(C)(C)COC(=O)[C@@H]4CCCN(N4)C2=O)C3)C(C)C)C1] |
| BB 260 | 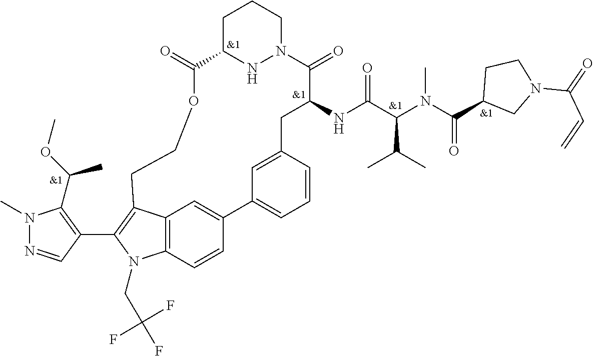 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)CCOC(=O)[C@@H]3CCCN(N3)C2=O)C(C)C)C1] |
| BB 261 | 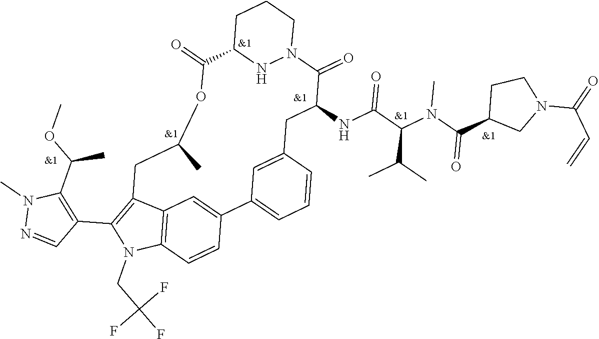 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)C[C@H](C)OC(=O)[C@@H]3CCCN(N3)C2=O)C(C)C)C1] |
| BB 262 | 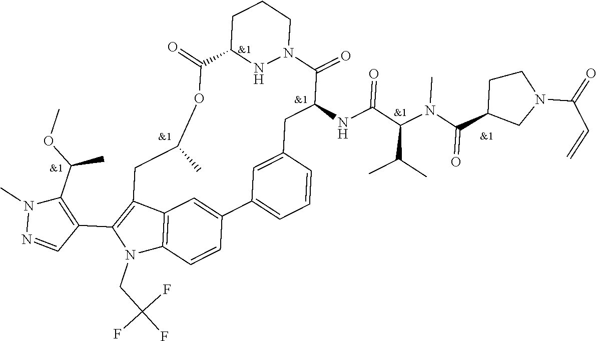 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)C[C@@H](C)OC(=O)[C@@H]3CCCN(N3)C2=O)C(C)C)C1] |
| BB 263 | 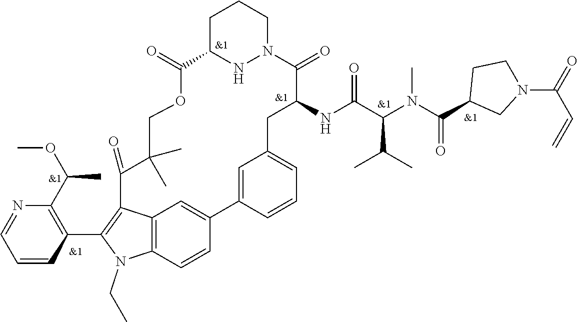 |
| BB 264 | 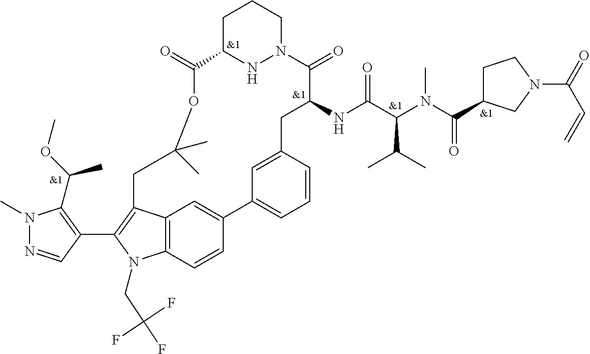 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)CC(C)(C)OC(=O)[C@@H]3CCCN(N3)C2=O)C(C)C)C1] |
| BB 265 | 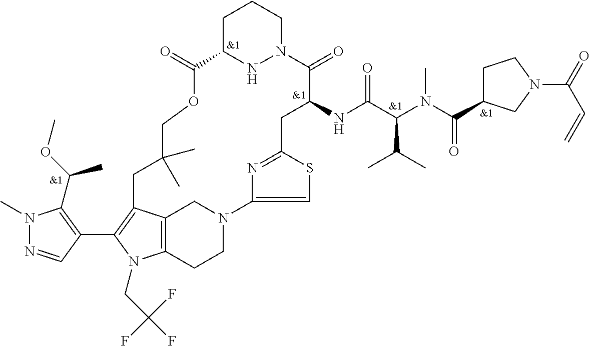 [SMILES: C=CC(=O)N1CC[C@H](C(=O)N(C)[C@H](C(=O)N[C@H]2Cc3nc(cs3)N3CCc4c(c(c(-c5cnn(C)c5[C@H](C)OC)n4CC(F)(F)F)CC(C)(C)COC(=O)[C@@H]4CCCN(N4)C2=O)C3)C(C)C)C1] |
| BB 266 | 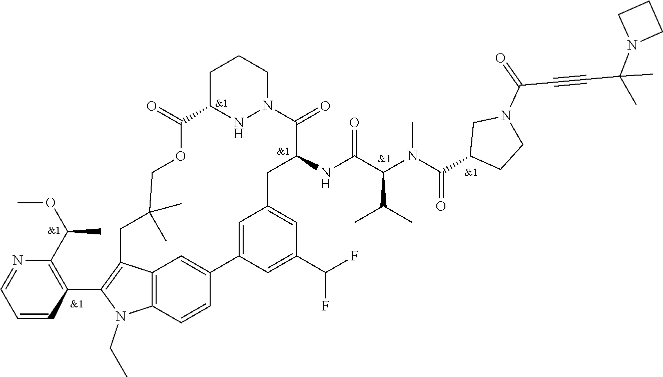 |
| BB 267 | 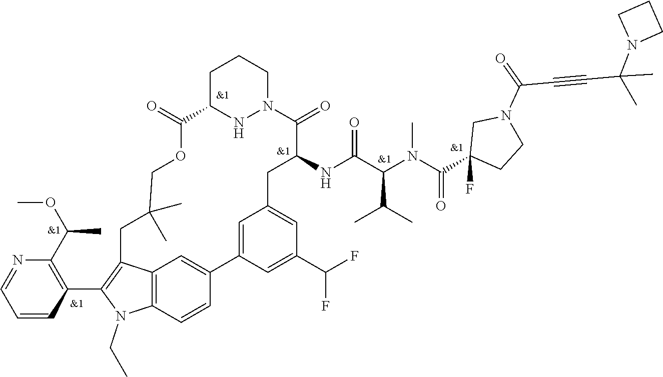 |
| BB 268 | 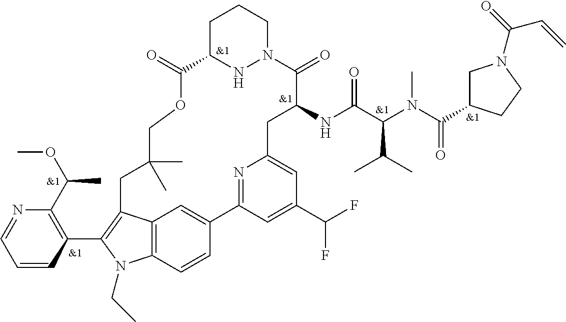 |
| BB 269 | 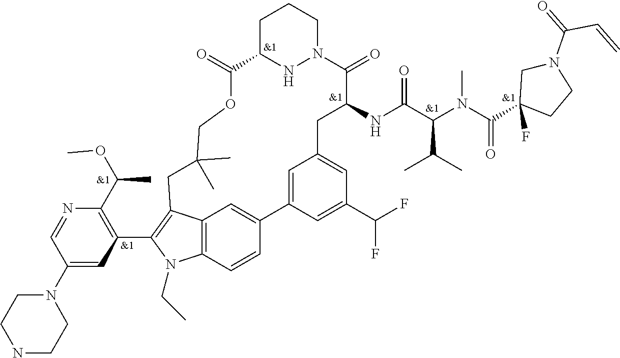 |
| BB 270 | 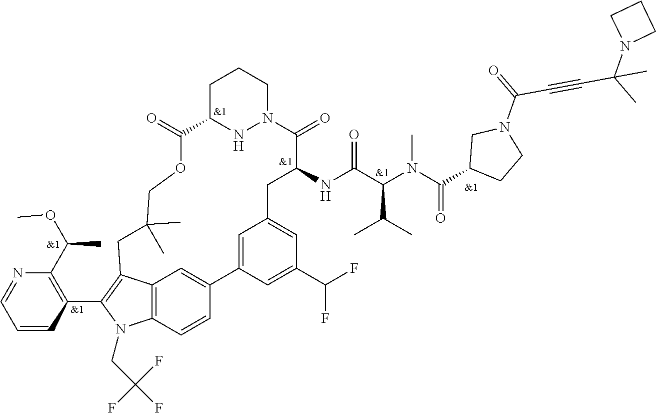 |
| BB 271 | 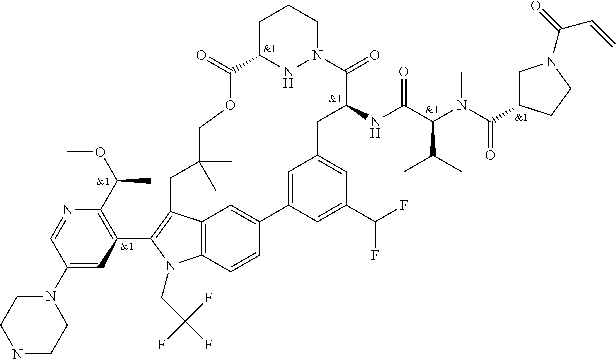 |
| BB 272 | 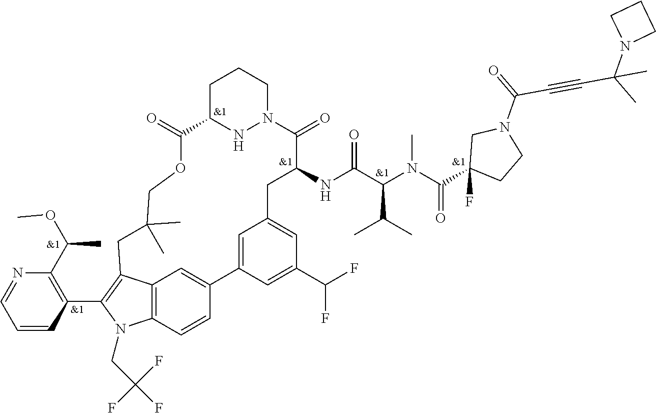 |
| BB 273 | 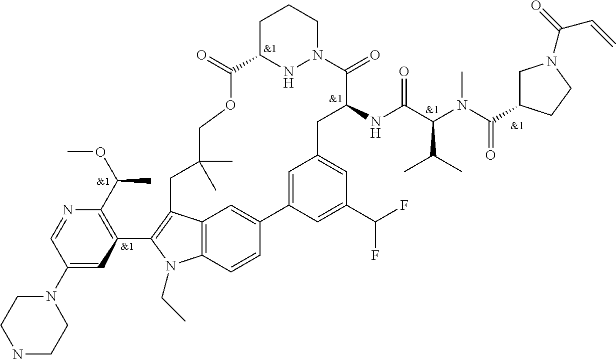 |
| BB 274 | 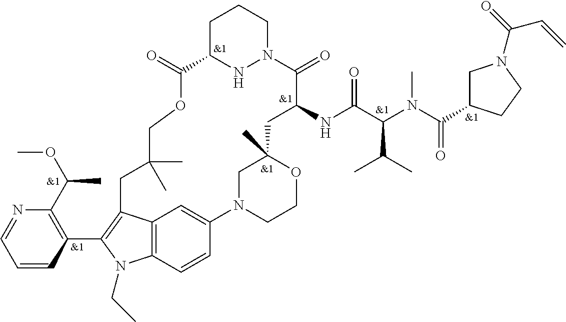 |
| BB 275 | 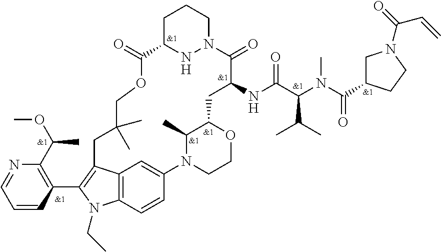 |
| BB 276 | 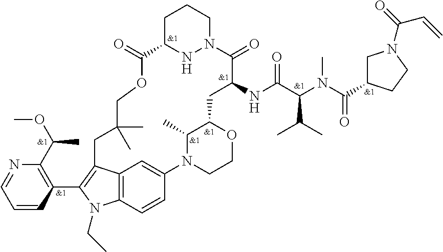 |
| BB 277 | 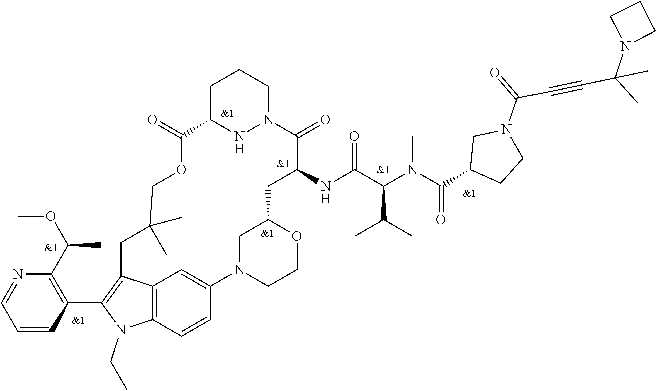 |
| BB 278 | 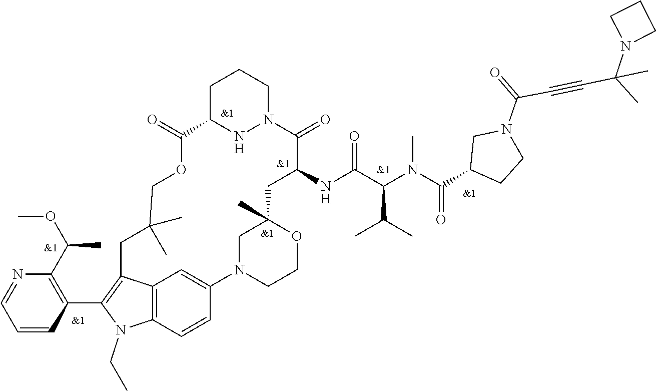 |
| BB 279 | 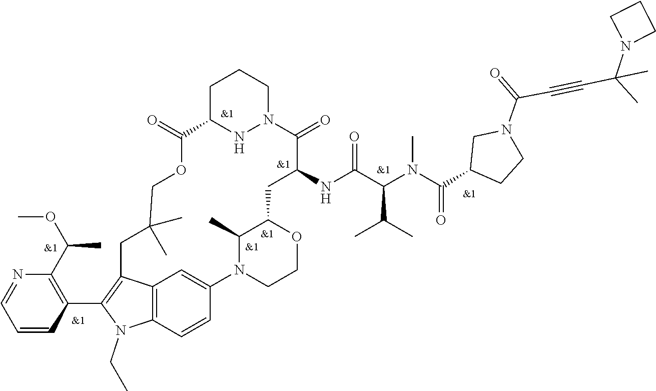 |
| BB 280 | 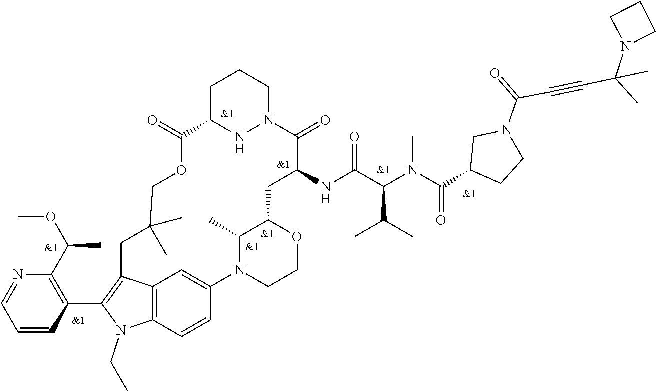 |
| BB 282 | 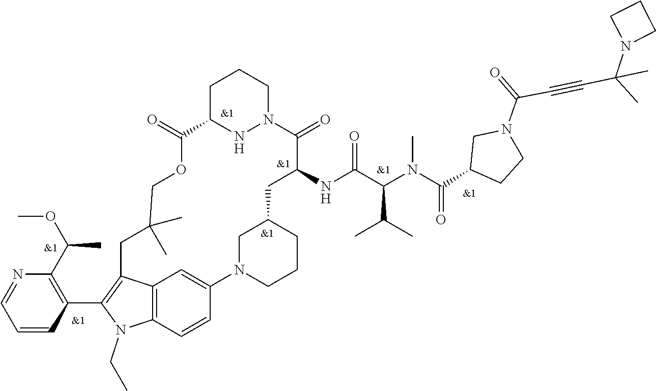 |
| BB 283 | 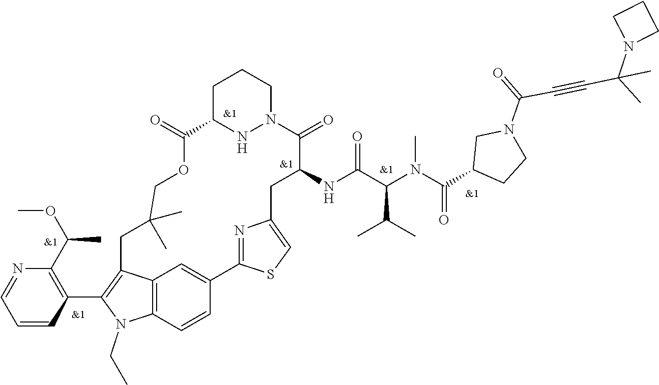 |
| BB 284 | 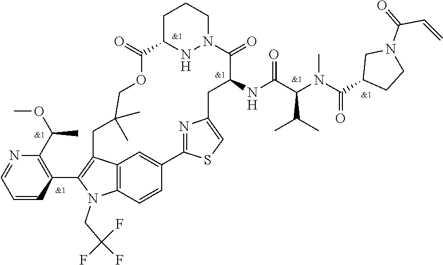 |
| BB 285 | 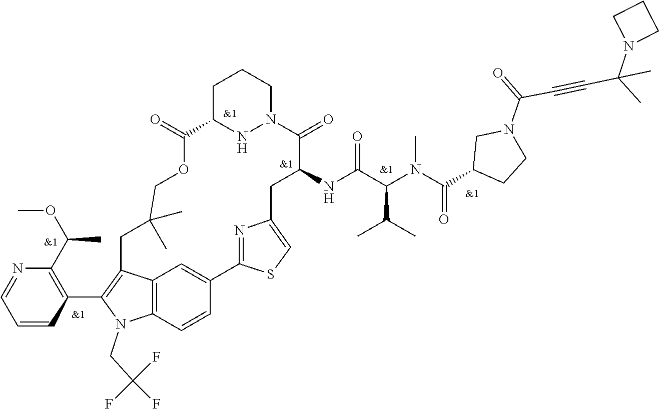 |
| BB 286 | 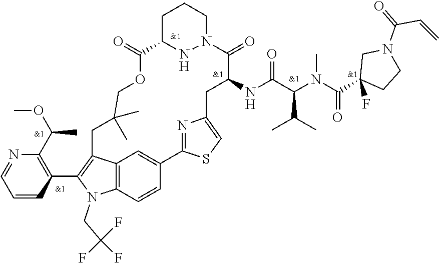 |
| BB 287 | 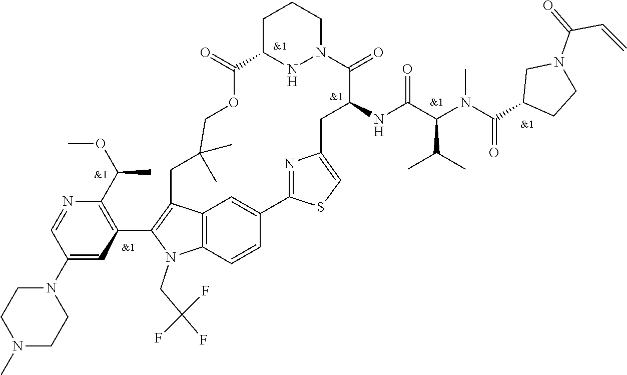 |
| BB 288 | 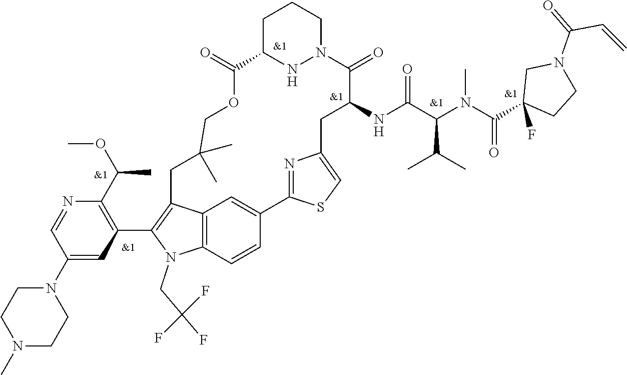 |
| BB 289 | 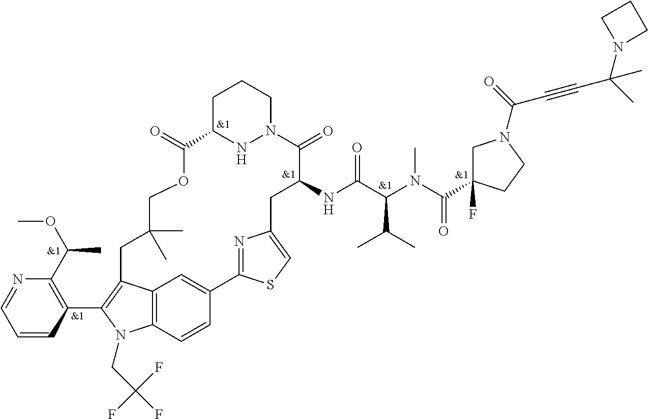 |
| BB 290 | 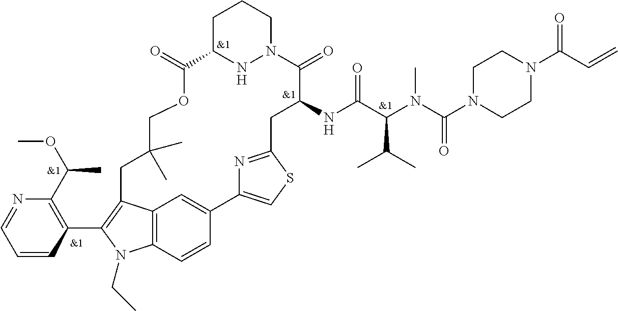 |
| BB 291 | 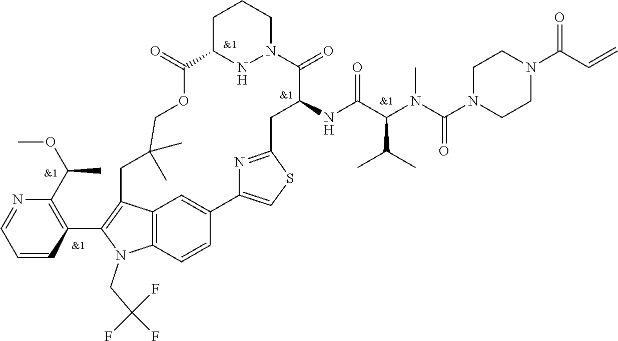 |
| BB 292 | 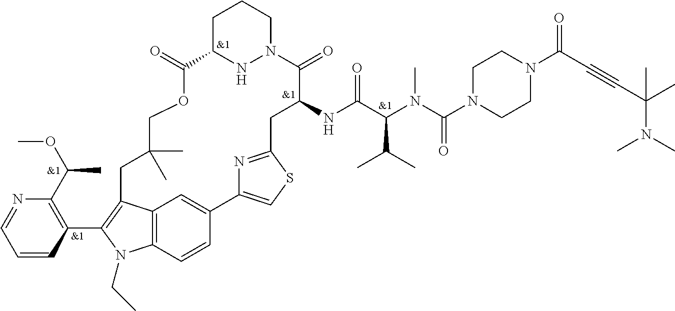 |
| BB 293 | 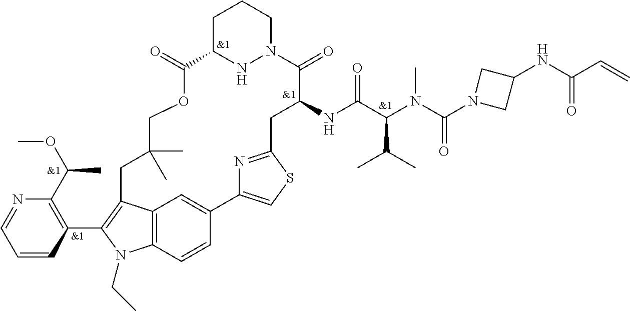 |
| BB 294 | 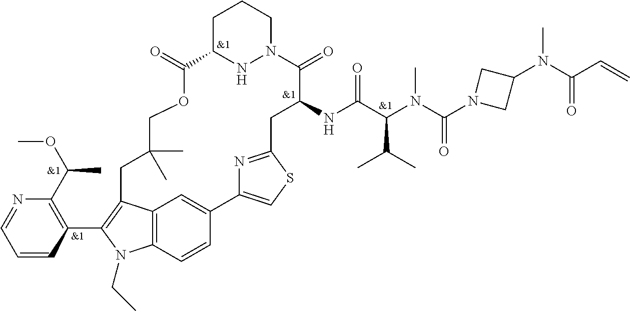 |
| BB 295 | 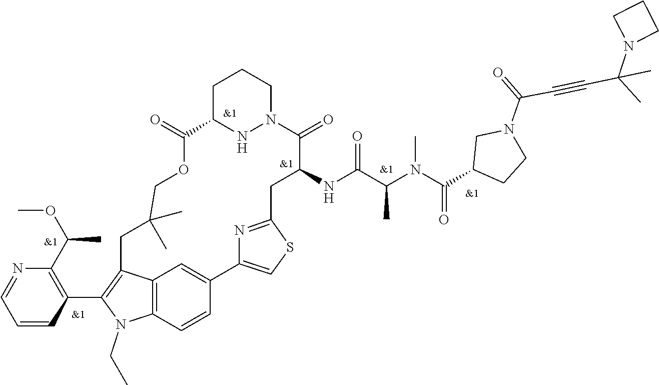 |
| BB 296 | 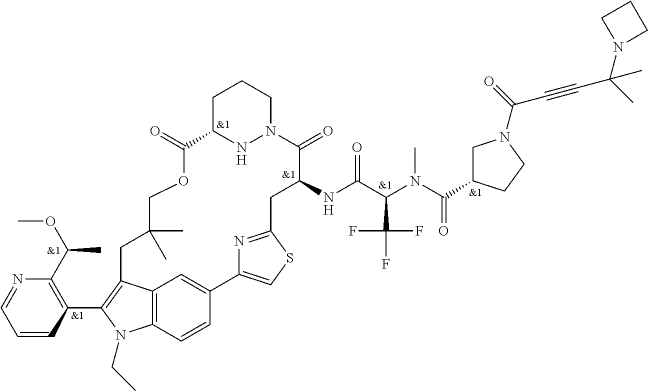 |
| BB 297 | 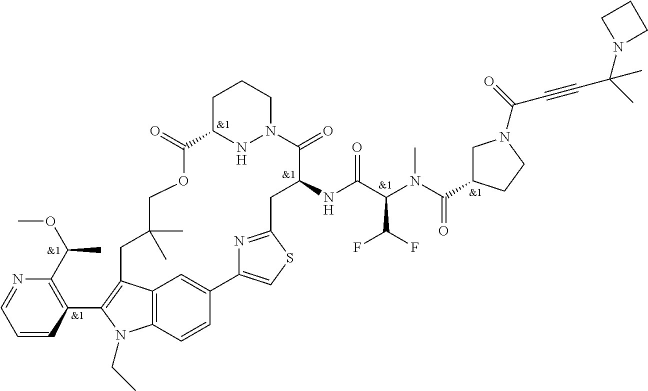 |
| BB 298 | 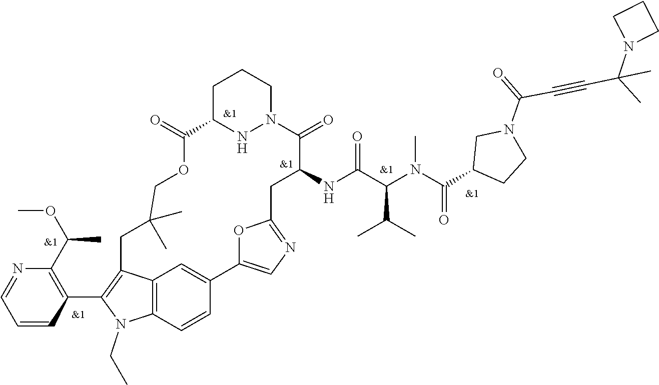 |
| BB 299 | 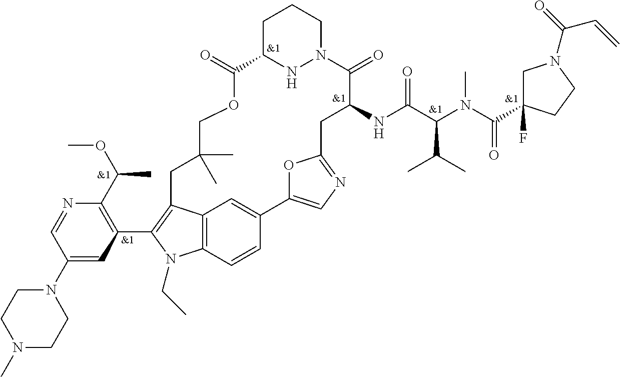 |
| BB 300 | 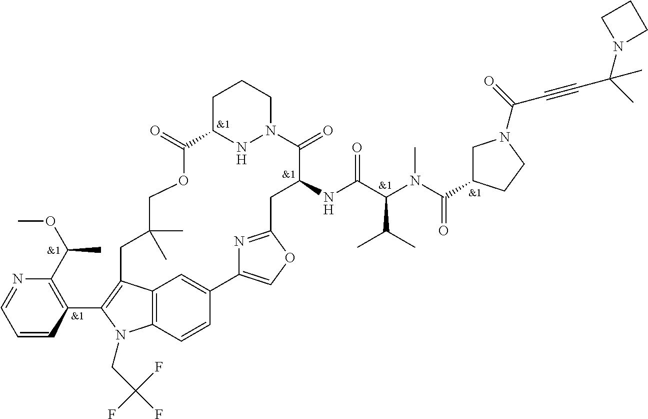 |
| BB 301 | 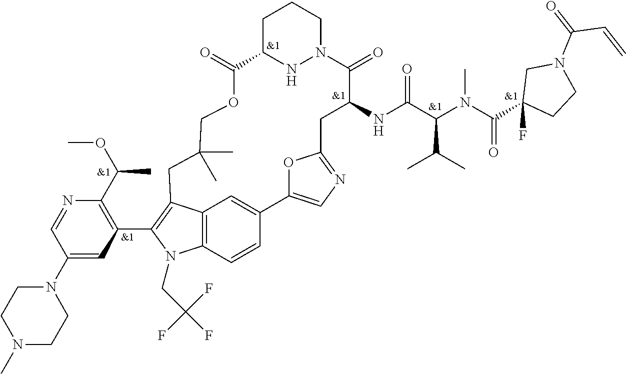 |
| BB 302 | 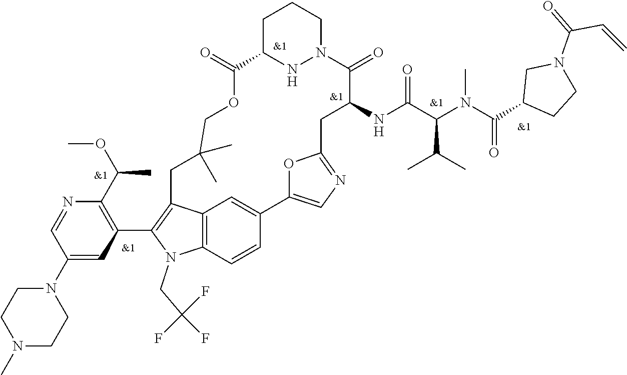 |
| BB 303 | 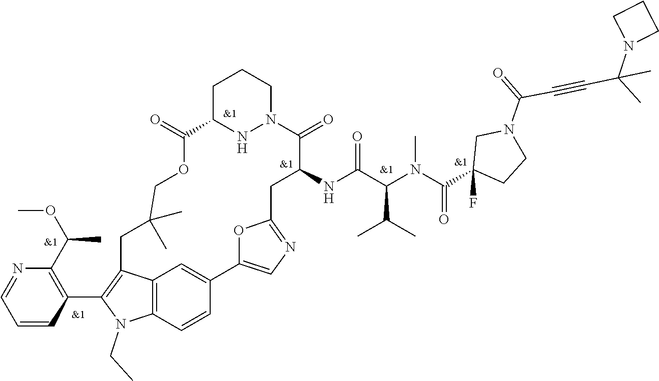 |
| BB 304 | 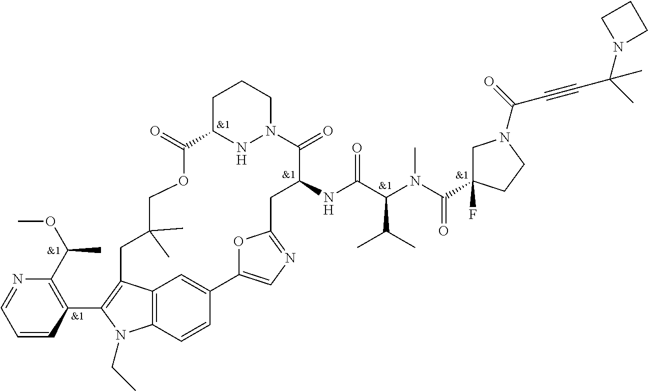 |
| BB 305 | 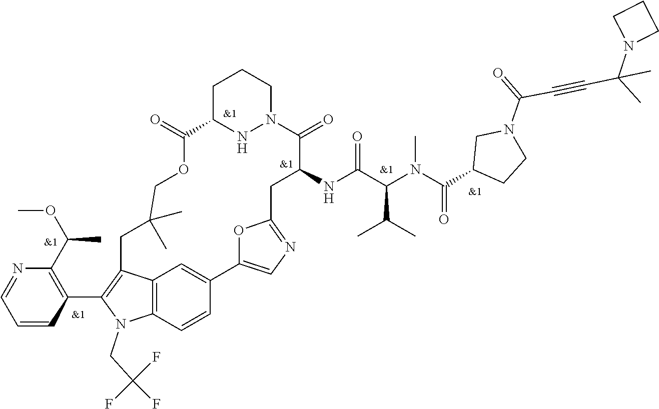 |
| BB 306 | 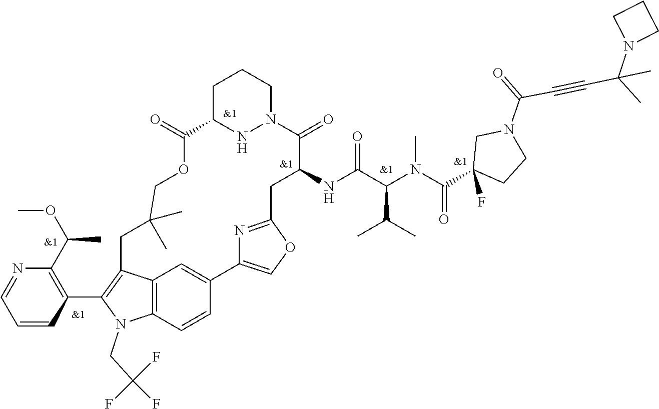 |
| BB 307 | 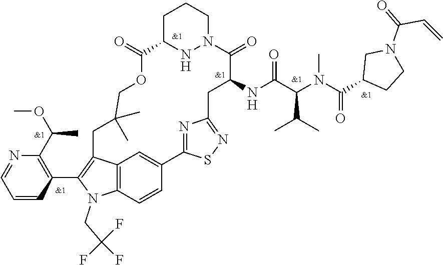 |
| BB 308 | 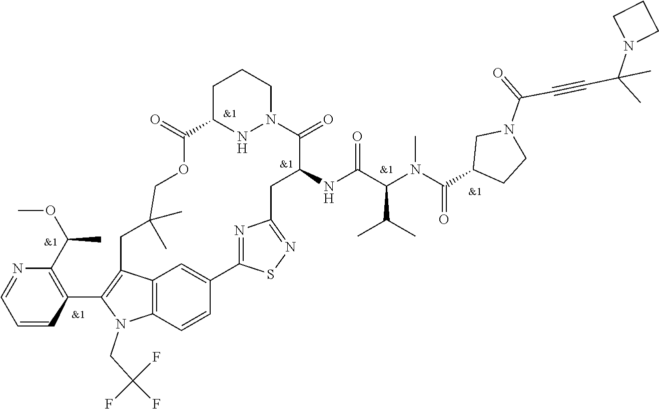 |
| BB 309 | 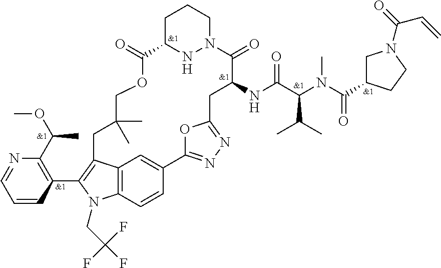 |
| BB 310 | 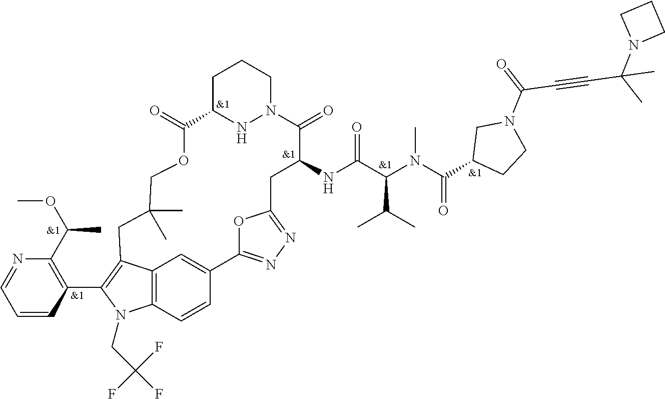 |
| BB 311 | 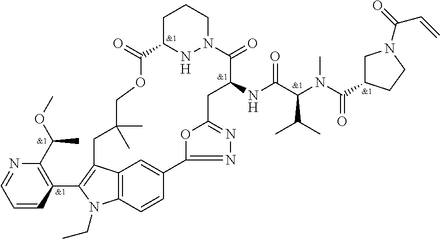 |
| BB 312 | 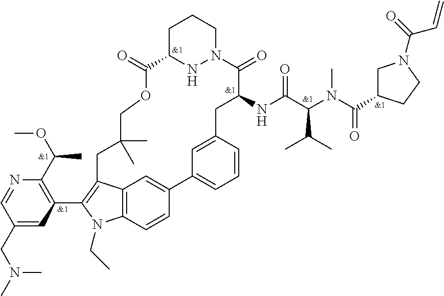 |
| BB 313 | 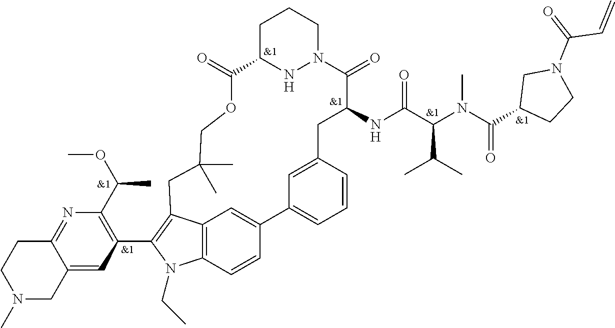 |
| BB 314 | 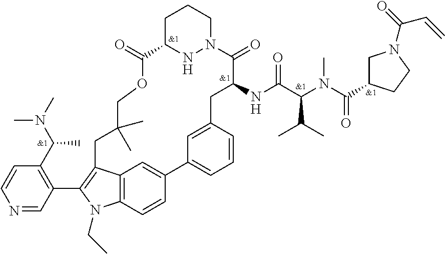 [SMILES: C=CC(=O)N1CC[C@H](C(=O)N(C)[C@H](C(=O)N[C@H]2Cc3cccc(c3)-c3ccc4c(c3)c(c(-c3cnccc3[C@@H](C)N(C)C)n4CC)CC(C)(C)COC(=O)[C@@H]3CCCN(N3)C2=O)C(C)C)C1] |
| BB 315 | 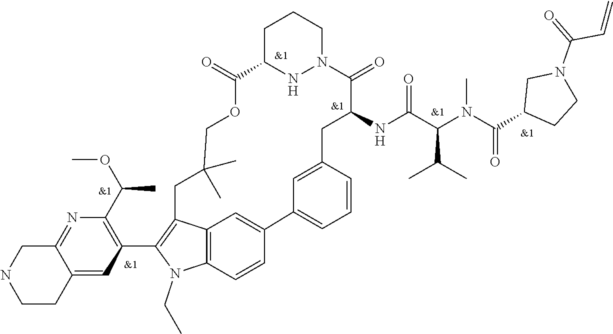 |
| BB 316 | 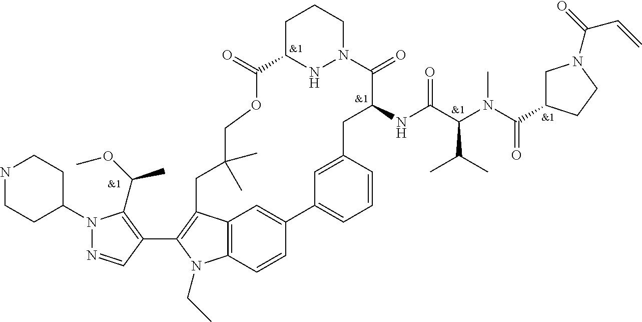 [SMILES: C=CC(=O)N1CC[C@H](C(=O)N(C)[C@H](C(=O)N[C@H]2Cc3cccc(c3)-c3ccc4c(c3)c(c(-c3cnn(C5CCNCC5)c3[C@H](C)OC)n4CC)CC(C)(C)COC(=O)[C@@H]3CCCN(N3)C2=O)C(C)C)C1] |
| BB 317 | 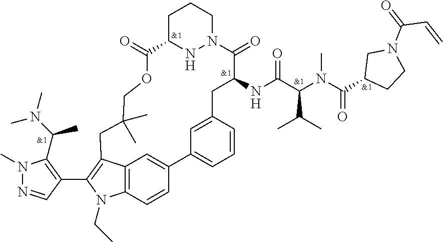 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)N(C)C)n4CC)CC(C)(C)COC(=O)[C@@H]3CCCN(N3)C2=O)C(C)C)C1] |
| BB 318 | 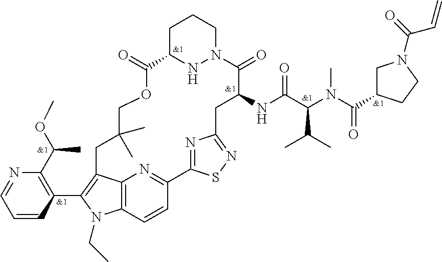 |
| BB 319 | 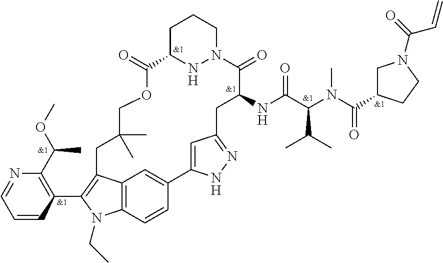 |
| BB 320 | 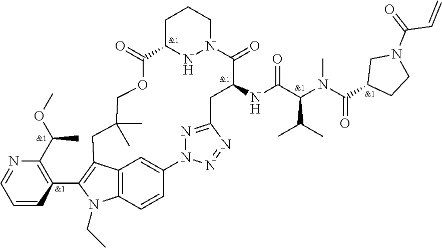 |
| BB 321 | 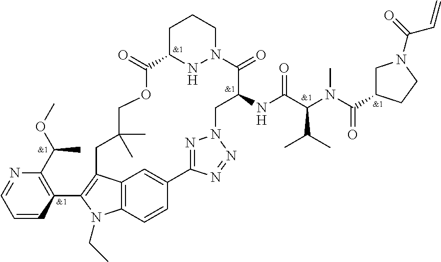 |
| BB 322 | 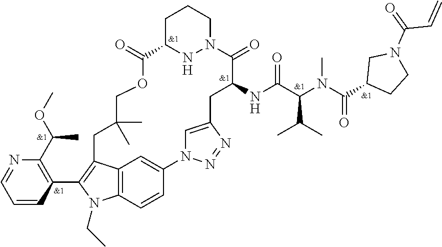 |
| BB 323 | 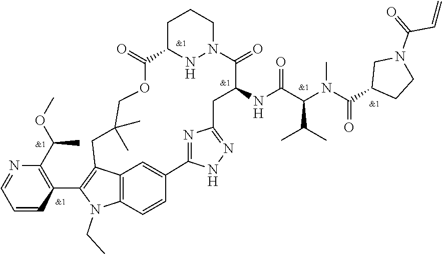 |
| BB 324 | 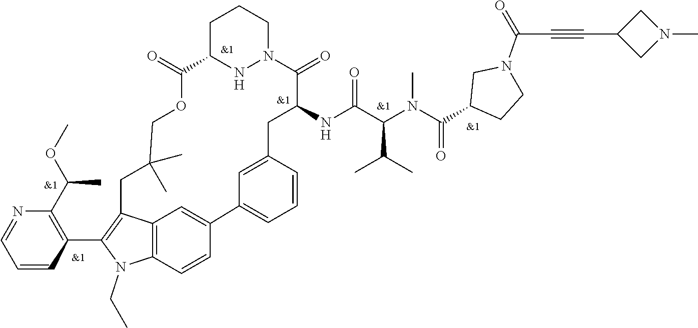 |
| BB 325 | 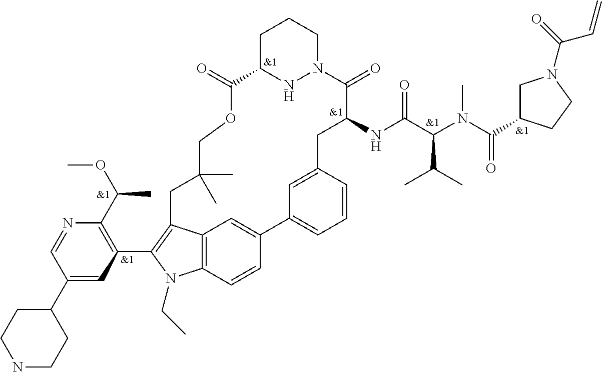 |
| BB 326 | 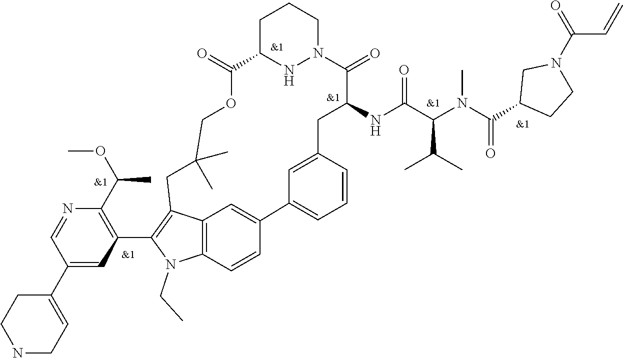 |
| BB 327 | 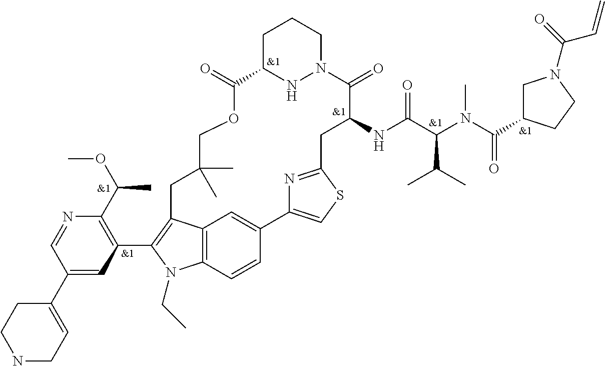 |
| BB 328 | 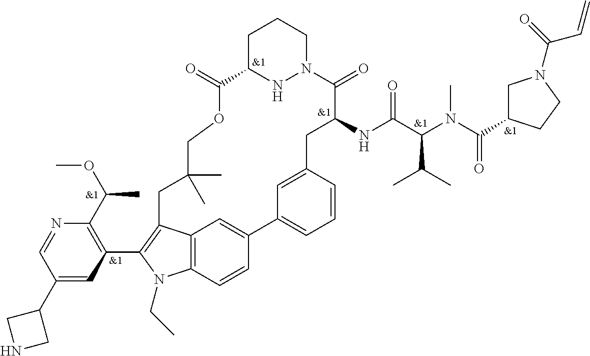 |
| BB 329 | 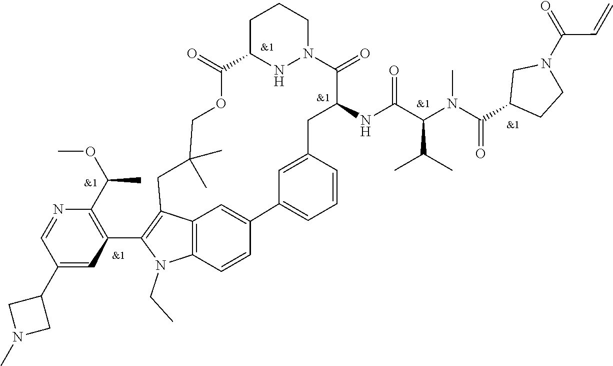 |
| BB 330 | 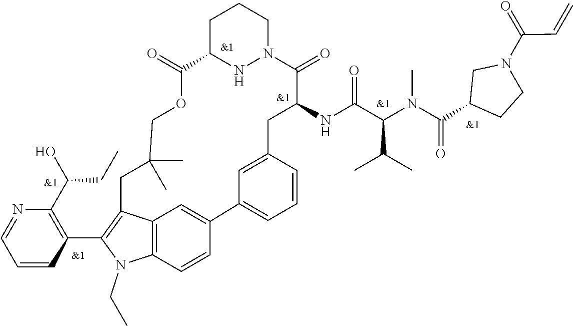 |
| BB 331 | 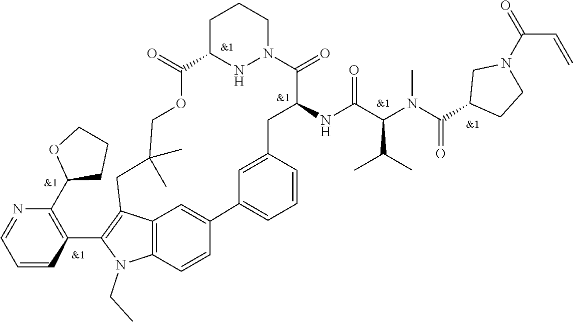 |
| BB 332 | 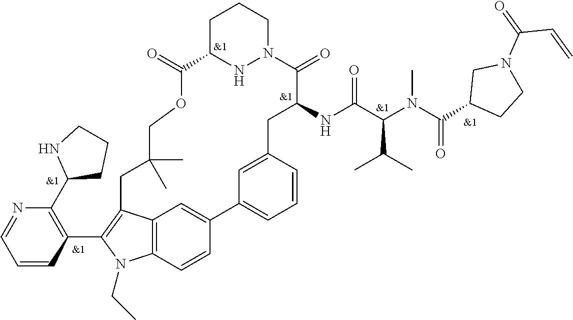 |
| BB 333 | 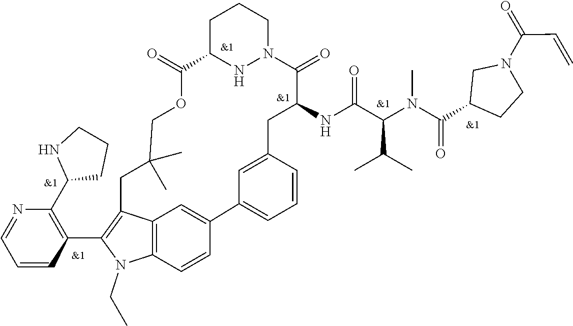 |
| BB 334 | 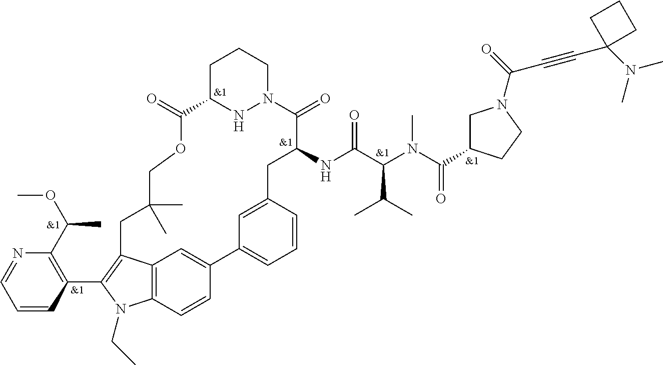 |
| BB 335 | 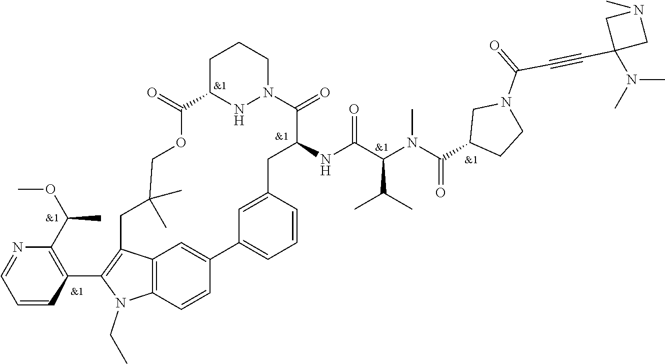 |
| BB 336 | 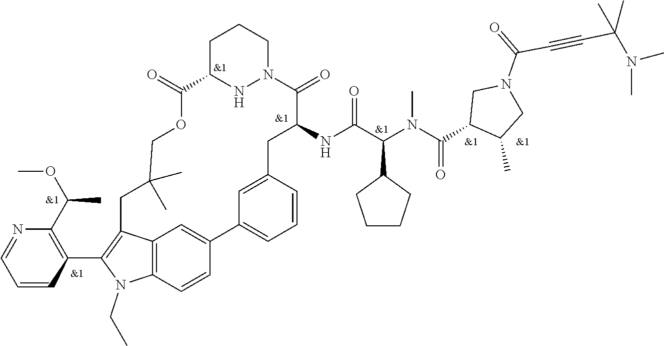 |
| BB 337 | 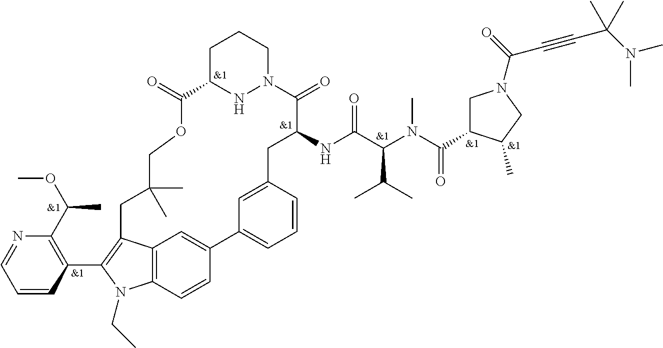 |
| BB 338 | 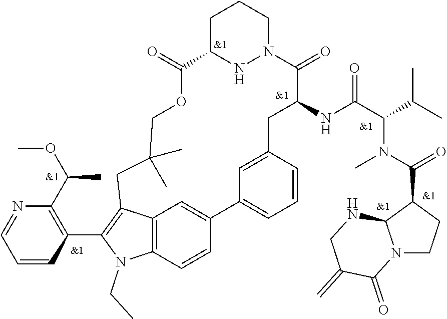 |
| BB 339 | 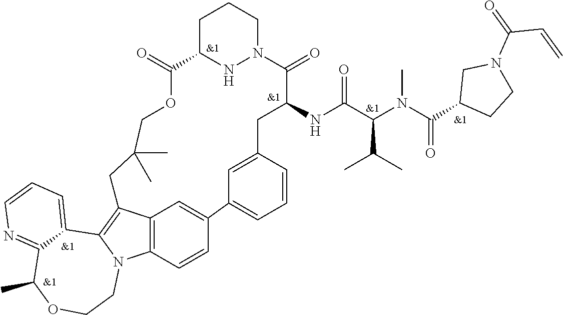 |
| BB 340 | 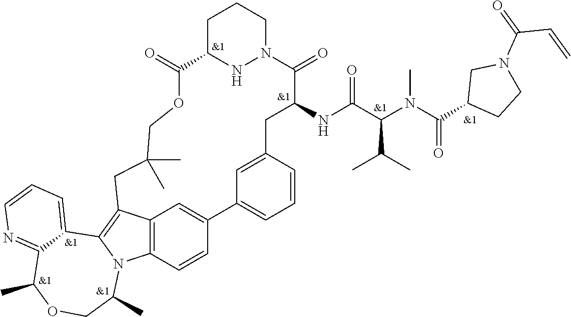 |
| BB 341 | 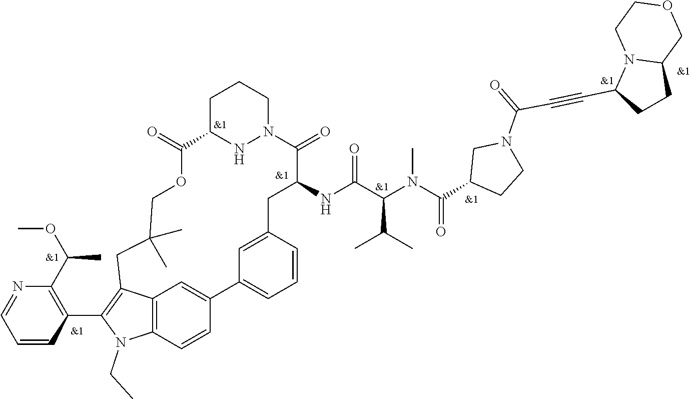 |
| BB 342 | 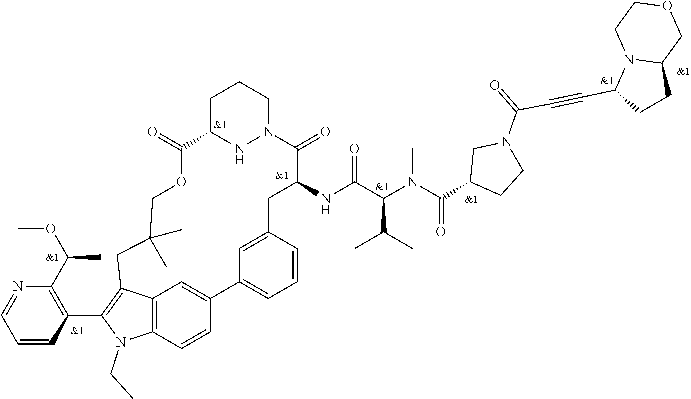 |
| BB 343 | 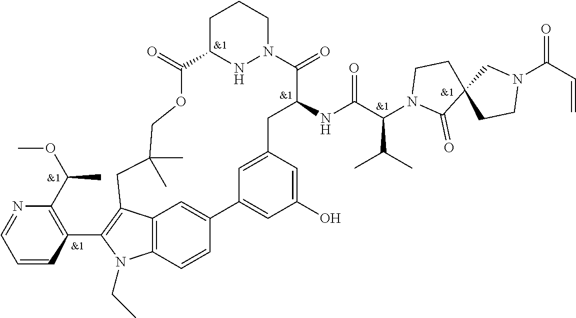 |
| BB 344 | 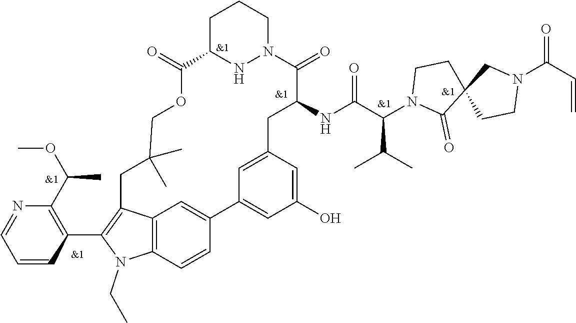 |
| BB 345 | 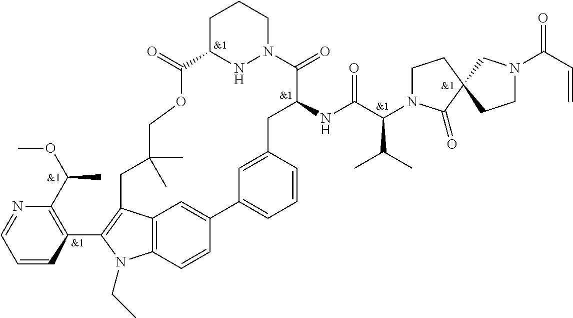 |
| BB 346 | 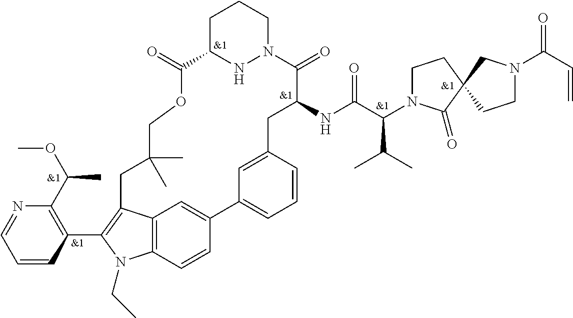 |
| BB 347 | 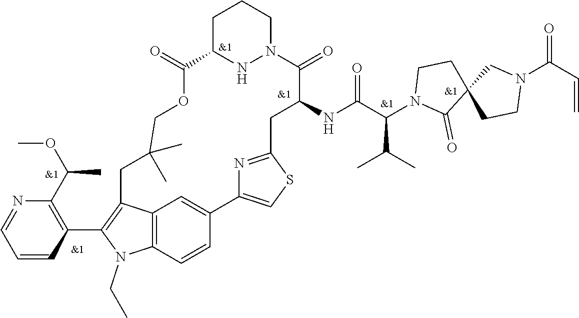 |
| BB 348 | 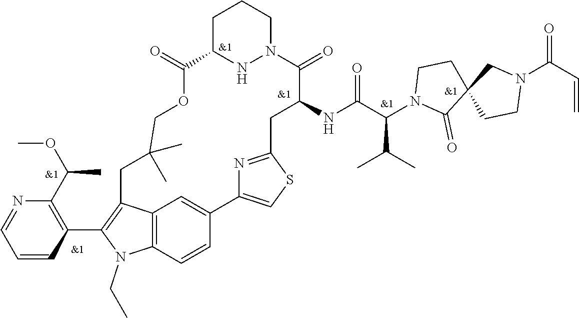 |
| BB 349 | 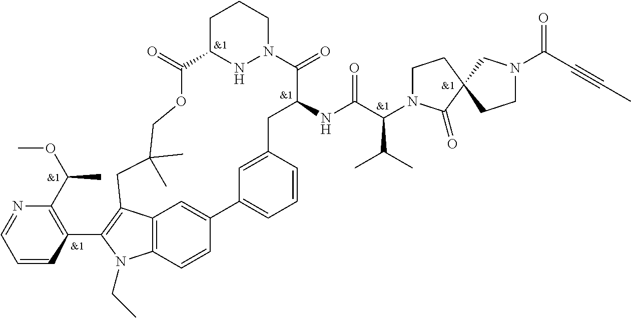 |
| BB 350 | 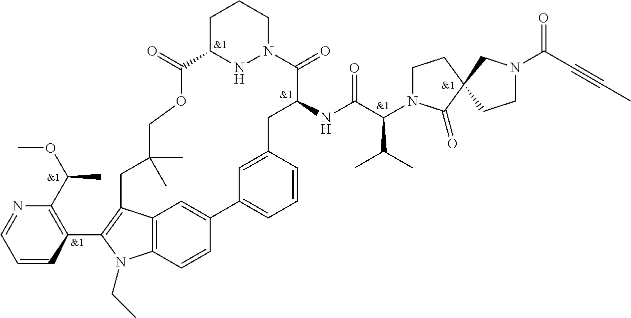 |
| BB 351 | 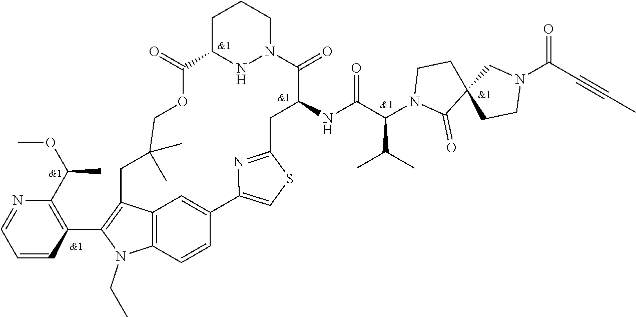 |
| BB 352 | 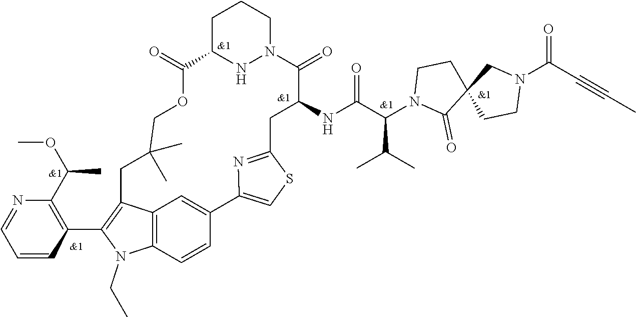 |
| BB 353 | 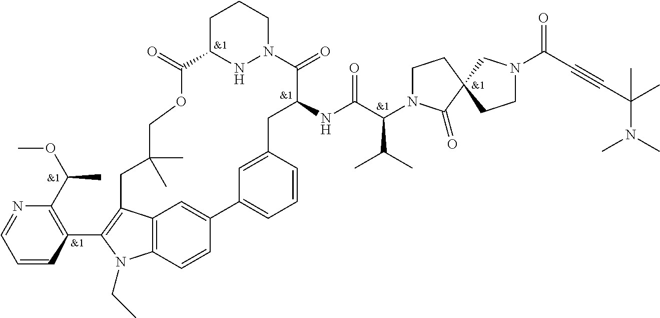 |
| BB 354 | 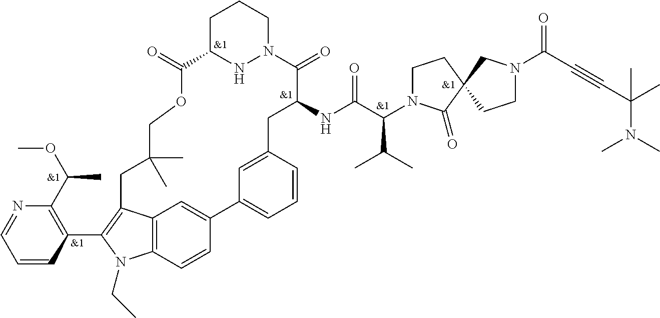 |
| BB 355 | 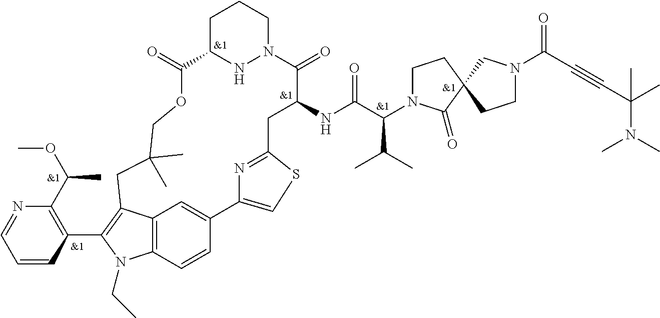 |
| BB 356 | 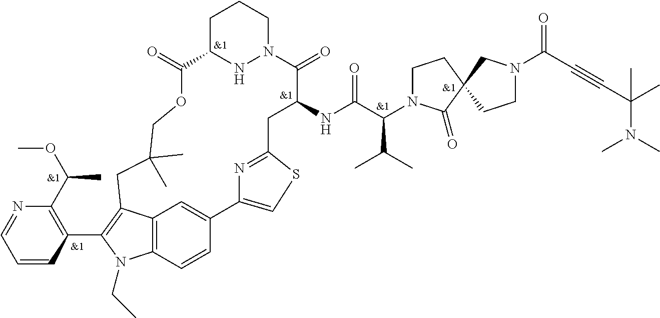 |
| BB 357 | 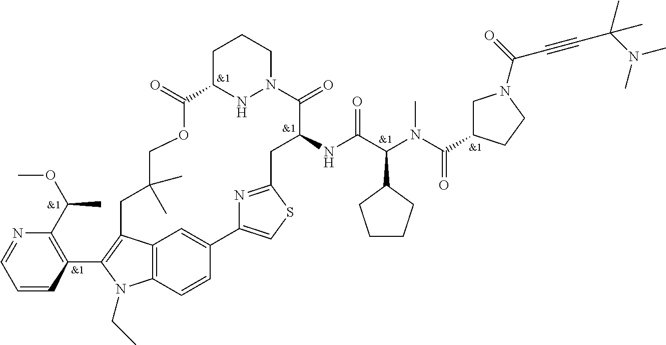 |
| BB 358 | 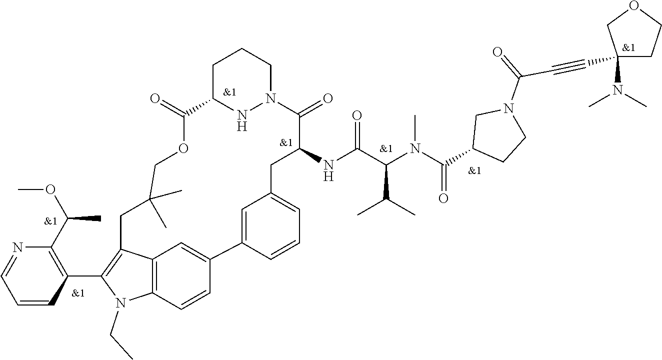 |
| BB 359 | 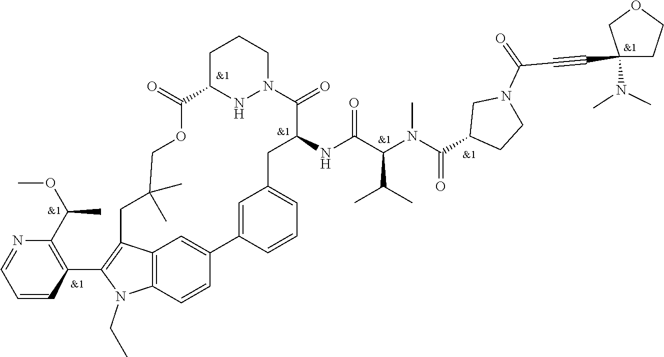 |
| BB 360 | 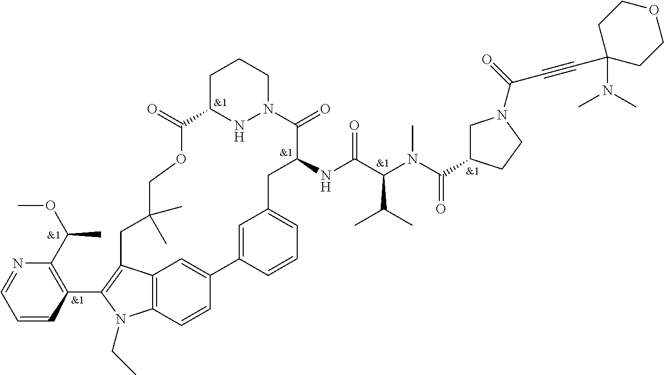 |
| BB 361 | 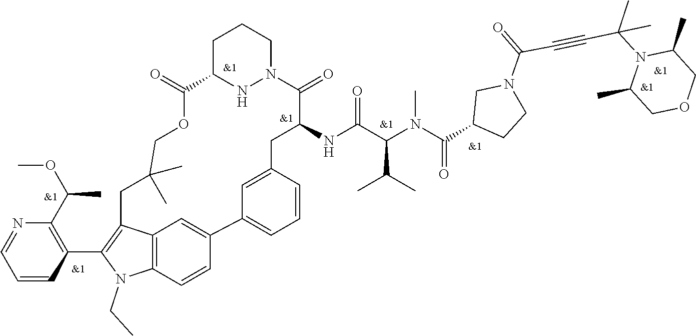 |
| BB 362 | 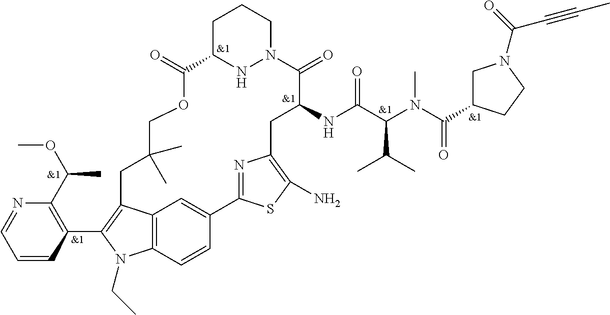 |
| BB 363 | 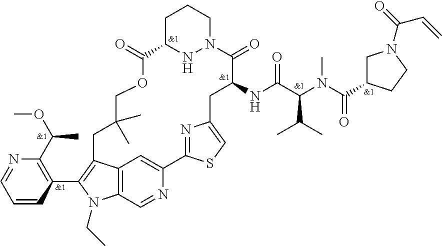 |
| BB 364 | 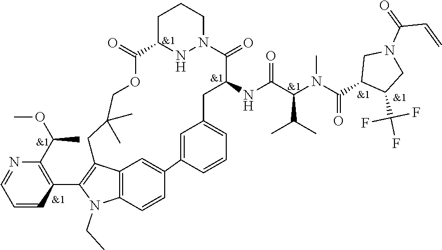 |
| BB 365 | 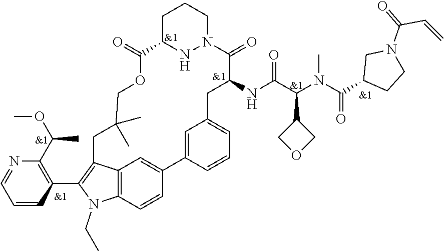 |
| BB 366 | 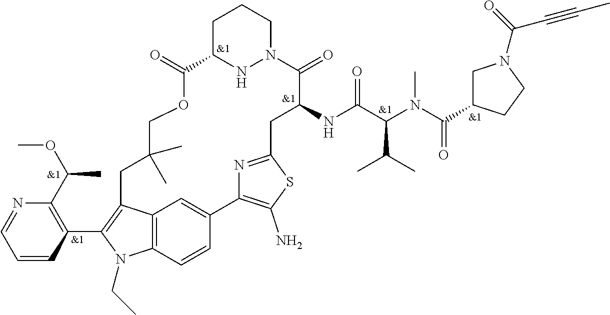 |
| BB 367 | 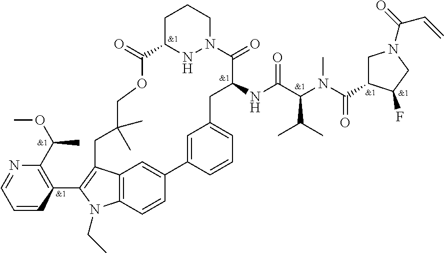 |
| BB 368 | 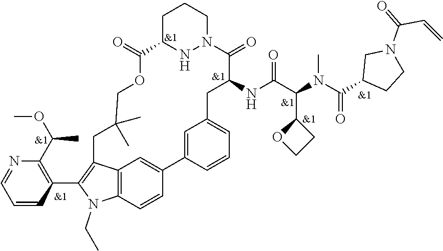 |
| BB 369 | 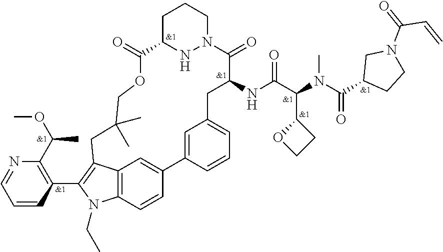 |
| BB 370 | 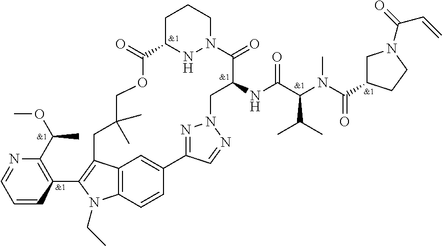 |
| BB 371 | 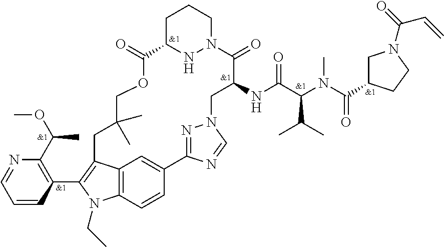 |
| BB 372 | 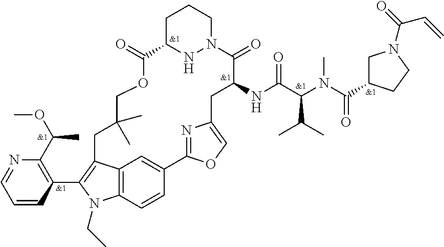 |
| BB 373 | 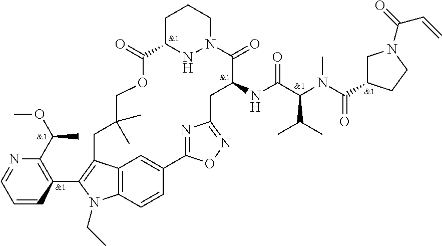 |
| BB 374 | 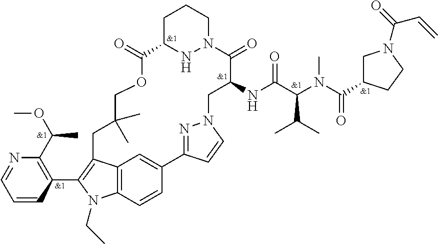 |
| BB 375 | 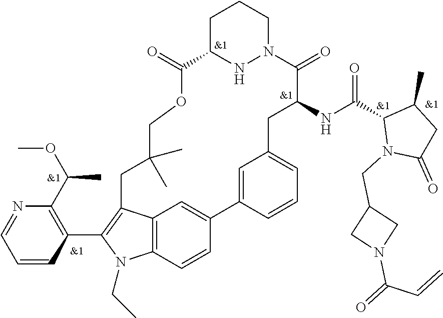 |
| BB 376 | 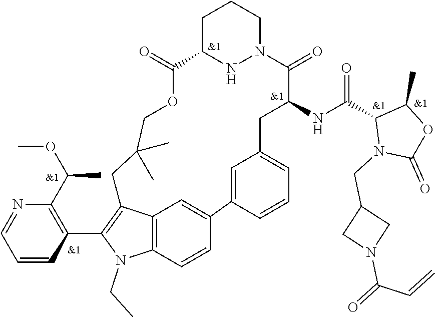 |
| BB 377 | 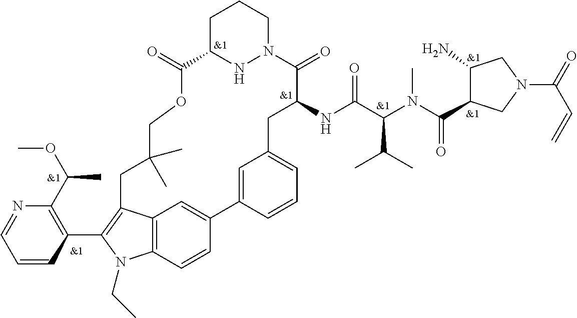 |
| BB 378 | 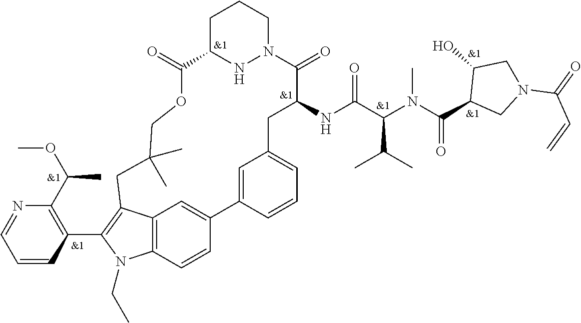 |
| BB 379 | 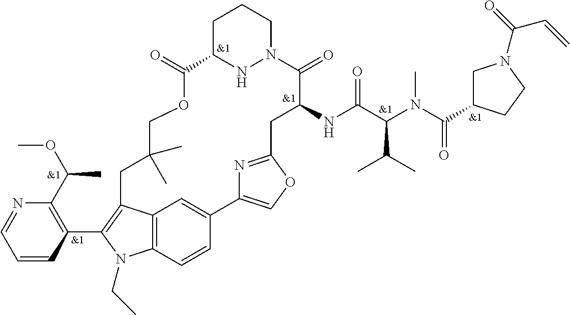 |
| BB 380 | 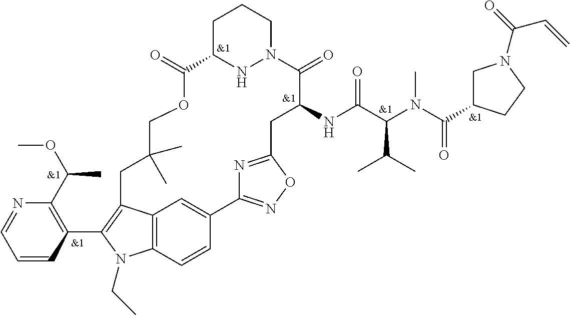 |
| BB 381 | 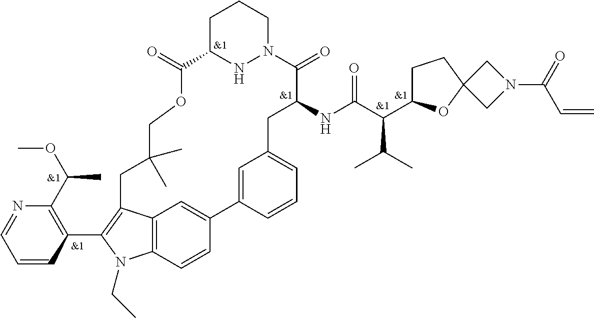 |
| BB 382 | 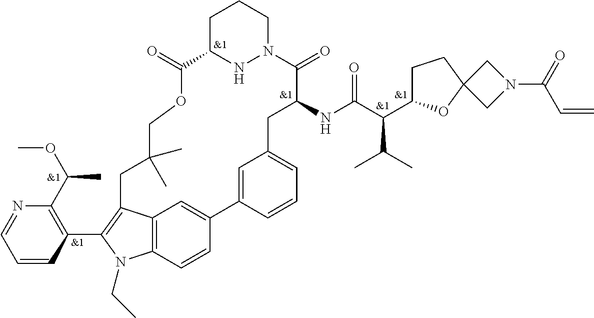 |
| BB 383 | 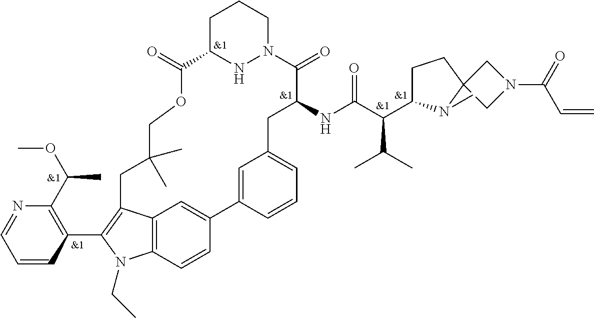 |
| BB 384 | 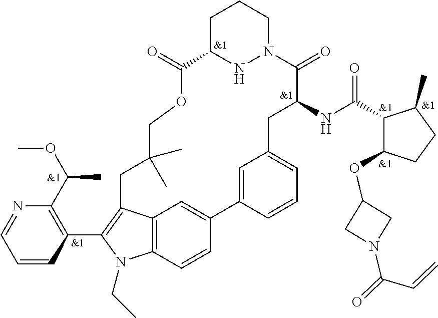 |
| BB 385 | 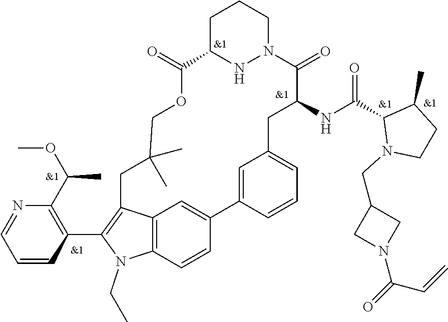 |
| BB 386 | 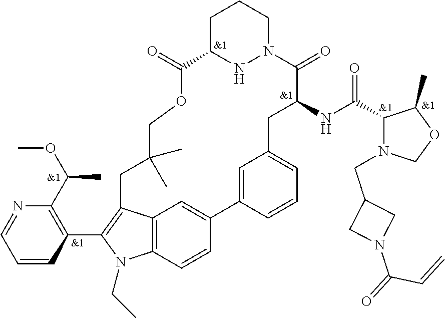 |
| BB 387 | 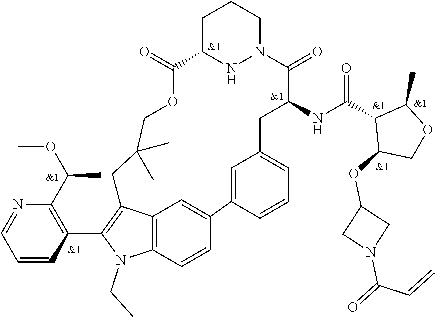 |
| BB 388 | 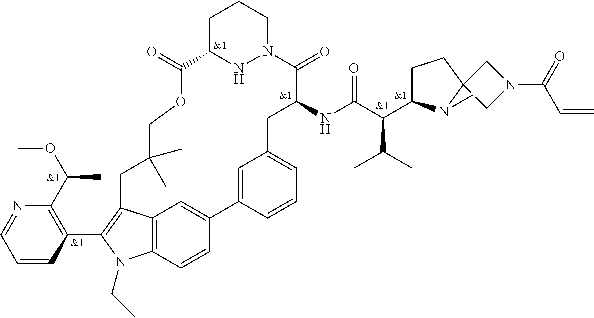 |
| BB 389 | 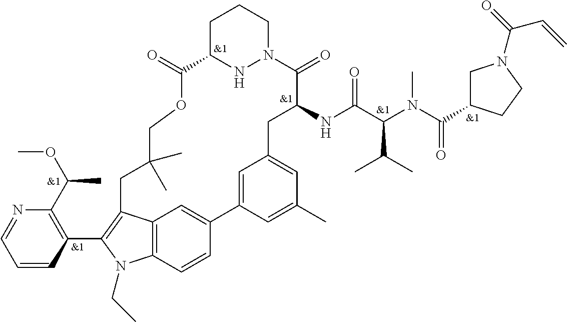 |
| BB 390 | 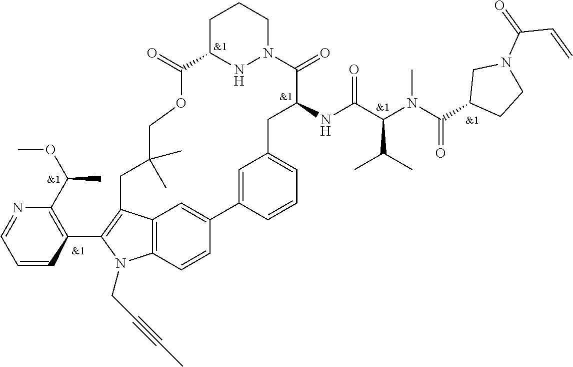 |
| BB 391 | 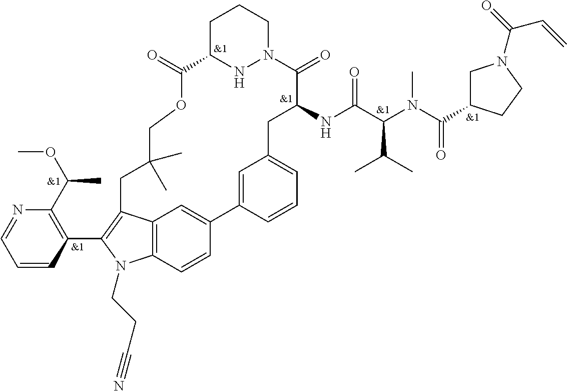 |
| BB 392 | 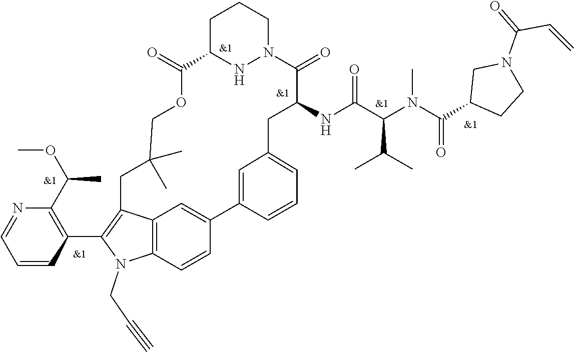 |
| BB 393 | 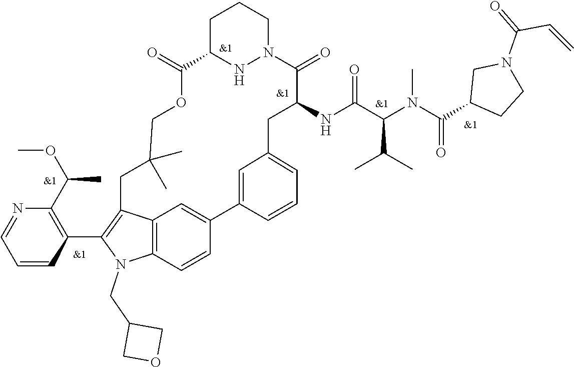 |
| BB 394 | 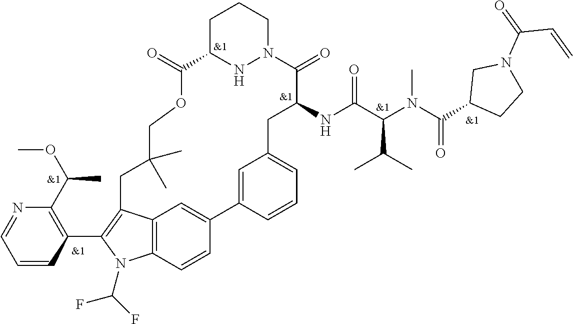 |
| BB 395 | 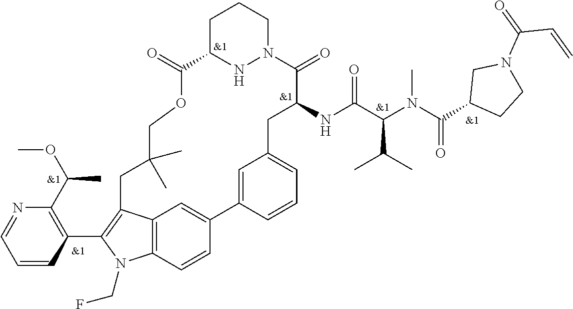 |
| BB 396 | 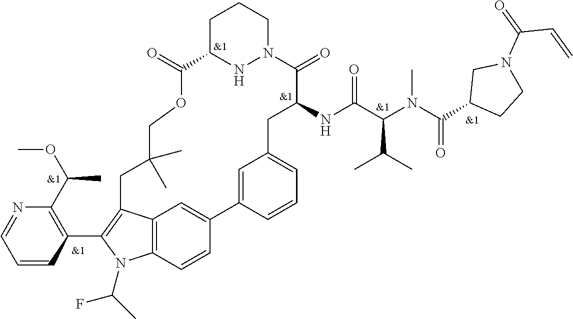 |
| BB 397 | 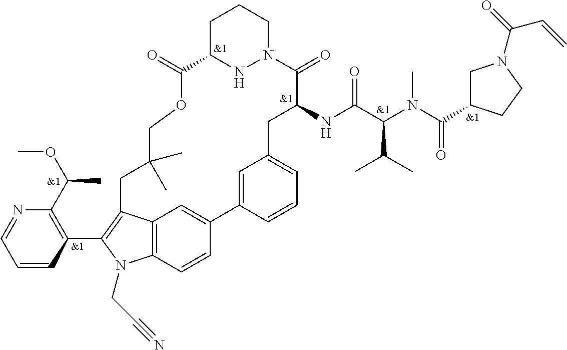 |
| BB 398 | 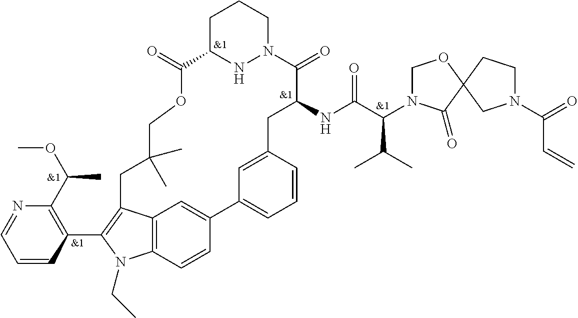 |
| BB 399 | 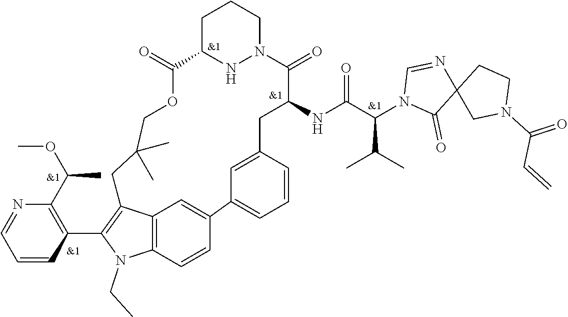 |
| BB 400 | 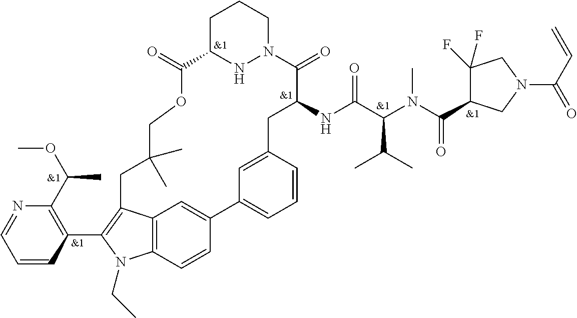 |
| BB 401 | 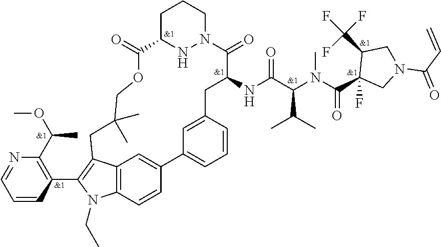 |
| BB 402 | 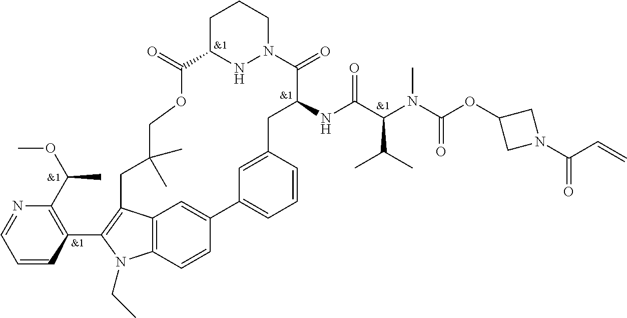 |
| BB 403 | 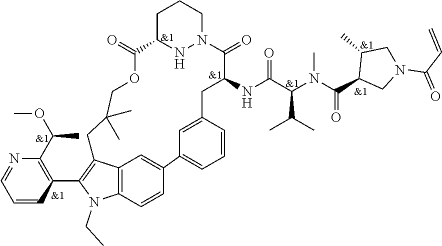 |
| BB 404 | 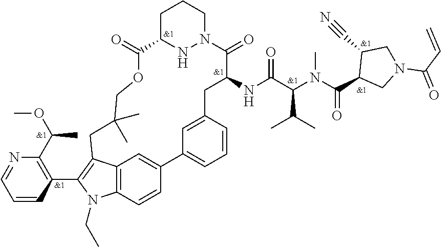 |
| BB 405 | 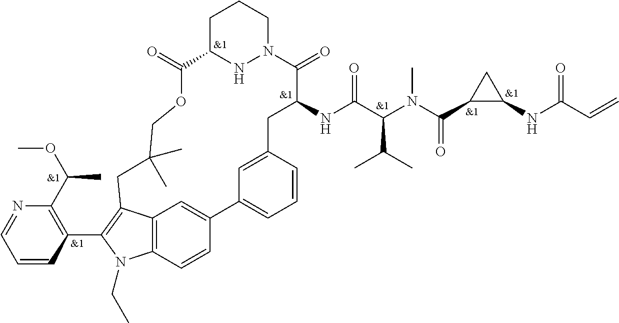 |
| BB 406 | 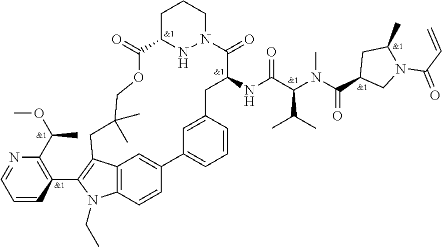 |
| BB 407 | 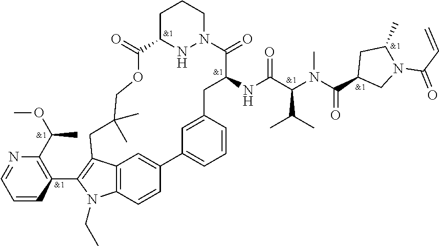 |
| BB 408 | 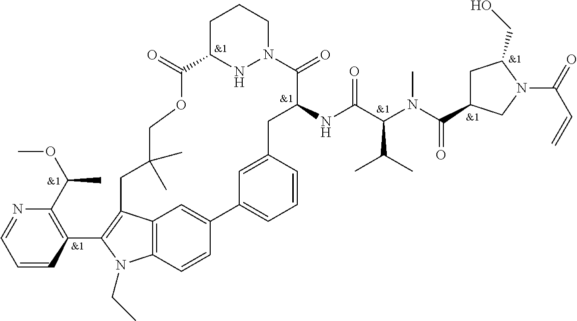 |
| BB 409 | 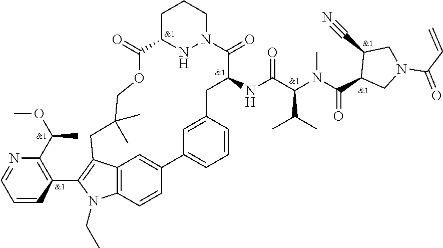 |
| BB 410 | 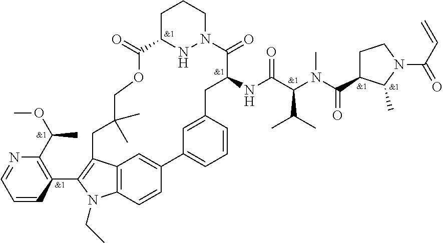 |
| BB 411 | 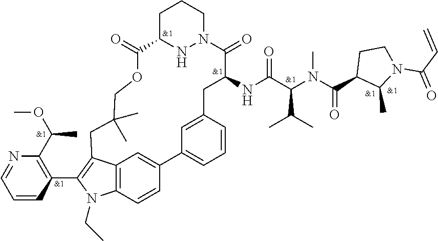 |
| BB 412 | 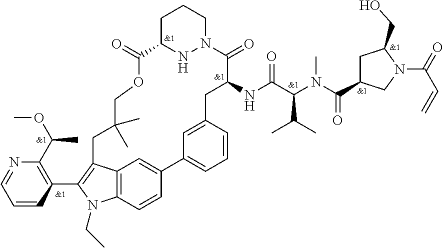 |
| BB 413 | 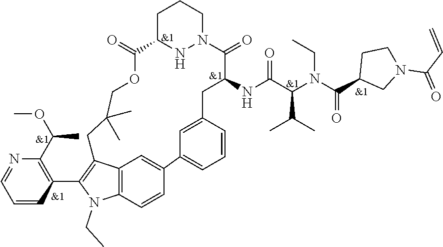 |
| BB 414 | 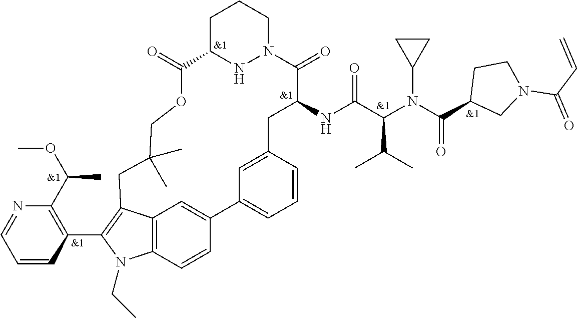 |
| BB 415 | 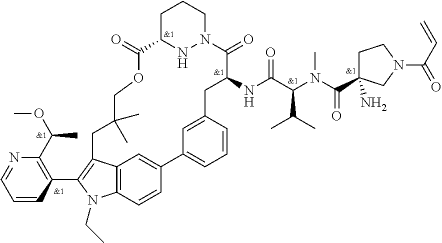 |
| BB 416 | 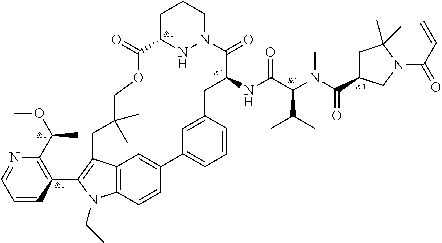 |
| BB 417 | 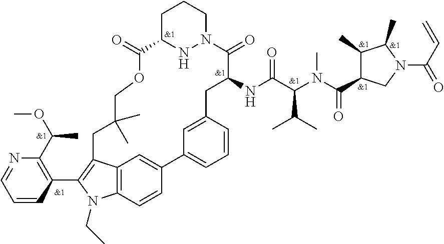 |
| BB 418 | 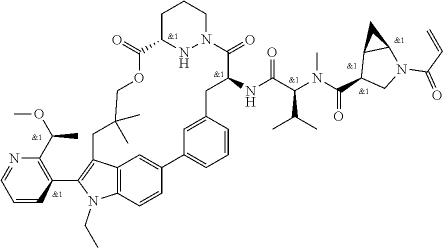 |
| BB 419 | 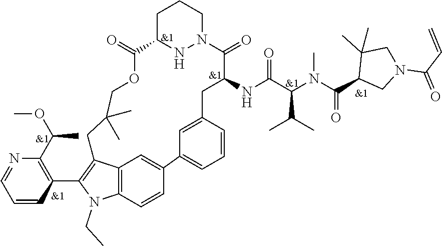 |
| BB 420 | 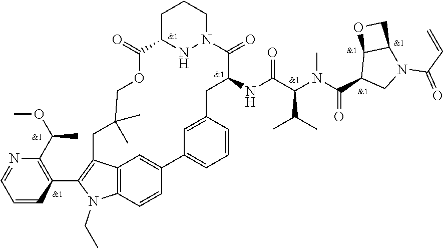 |
| BB 421 | 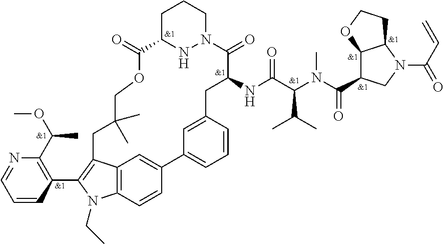 |
| BB 422 | 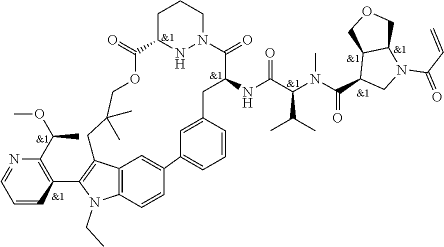 |
| BB 423 | 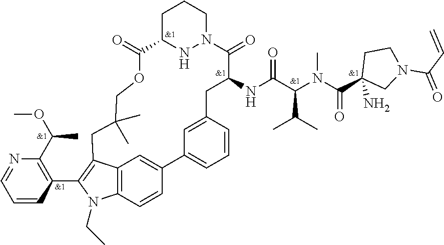 |
| BB 424 | 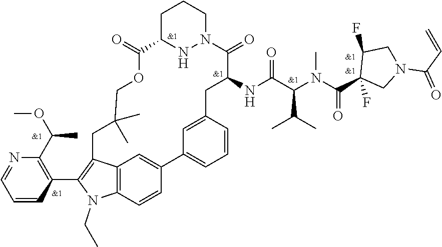 |
| BB 425 | 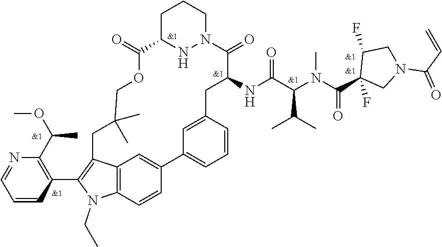 |

Note that some compounds are shown with bonds as flat or wedged. In some instances, the relative stereochemistry of stereoisomers has been determined; in some instances, the absolute stereochemistry has been determined.
All stereoisomers of the compounds of the foregoing table are contemplated by the present invention.
In particular embodiments, an atropisomer of a compound of the foregoing table is contemplated.

In some embodiments, the RAS inhibitor is or acts as a prodrug, such as with respect to administration to a cell or to a subject in need thereof.

Also provided are pharmaceutical compositions comprising a compound of the present invention, or a pharmaceutically acceptable salt thereof, and a pharmaceutically acceptable excipient.

In some embodiments, the RAS G12C inhibitor is provided as a conjugate, or salt thereof, comprising the structure of Formula BIV:

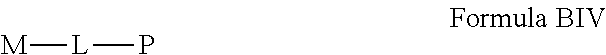

wherein L is a linker;
P is a monovalent organic moiety; and
M has the structure of Formula BVa:

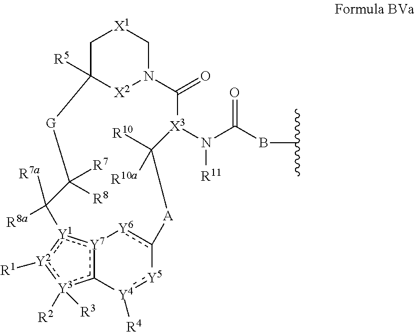

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′} C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is H, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl; or
R⁹ and R^{9′}, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments the conjugate, or salt thereof, comprises the structure of Formula BIV:

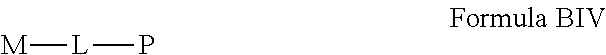

wherein L is a linker;
P is a monovalent organic moiety; and
M has the structure of Formula BVb:

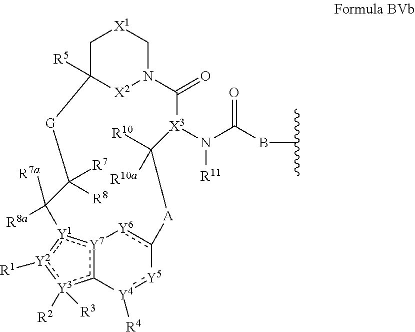

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments, the conjugate has the structure of Formula BIV:

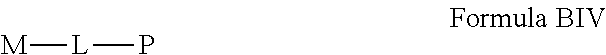

wherein L is a linker;
P is a monovalent organic moiety; and
M has the structure of Formula BVc:

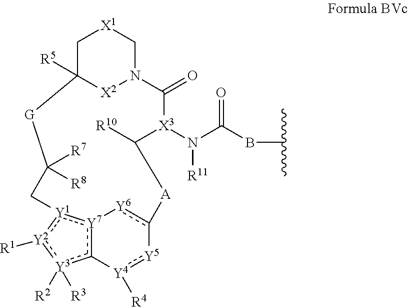

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′} C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments, the RAS G12C inhibitor has the structure of of Formula BIV:

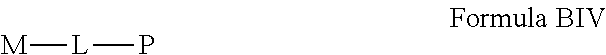

wherein L is a linker;
P is a monovalent organic moiety; and
M has the structure of Formula BVd:

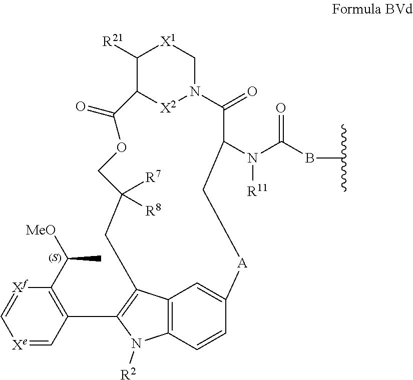

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
X¹ is optionally substituted C₁-C₂ alkylene, NR, O, or S(O)ₙ;
X² is O or NH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
R² is C₁-C₆ alkyl, C₁-C₆ fluoroalkyl, or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} and X^{f} are, independently, N or CH;
R¹¹ is hydrogen or C₁-C₃ alkyl; and
R²¹ is hydrogen or C₁-C₃ alkyl.

In some embodiments of formula BI and subformula thereof, X^{e} is N and X^{f} is CH. In some embodiments, X^{e} is CH and X^{f} is N.

In some embodiments, the RAS G12C inhibitor has the structure of of Formula BIV:

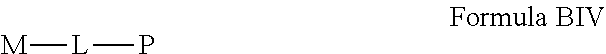

wherein L is a linker;
P is a monovalent organic moiety; and
M has the structure of Formula BVe:

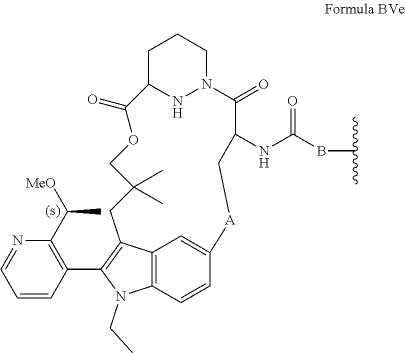

wherein A is optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of a conjugate of Formula BIV, the linker has the structure of Formula BII:

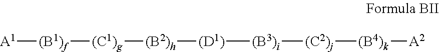

where A¹ is a bond between the linker and B; A² is a bond between P and the linker; B¹, B², B³, and B⁴ each, independently, is selected from optionally substituted C₁-C₂ alkylene, optionally substituted C₁-C₃ heteroalkylene, O, S, and NRN; RN is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)^{f}—(C¹)^{g}—(B²)^{h}— to —(B³)ⁱ—(C²)^{j}—(B⁴)^{k}-A².

In some embodiments of a conjugate of formula BIV, the monovalent organic moiety is a protein, such as a Ras protein. In some embodiments, the Ras protein is K-Ras G12C, K-Ras G13C, H-Ras G12C, H-Ras G13C, N-Ras G12C, or N-Ras G13C. Other Ras proteins are described herein. In some embodiments, the linker is bound to the monovalent organic moiety through a bond to a sulfhydryl group of an amino acid residue of the monovalent organic moiety. In some embodiments, the linker is bound to the monovalent organic moiety through a bond to a carboxyl group of an amino acid residue of the monovalent organic moiety.

The compounds described in Tables B1 and B2 may be made from commercially available starting materials or synthesized using known organic, inorganic, or enzymatic processes. The compounds of the present invention can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below or as described in WO 2021/091982, incorporated herein by reference.

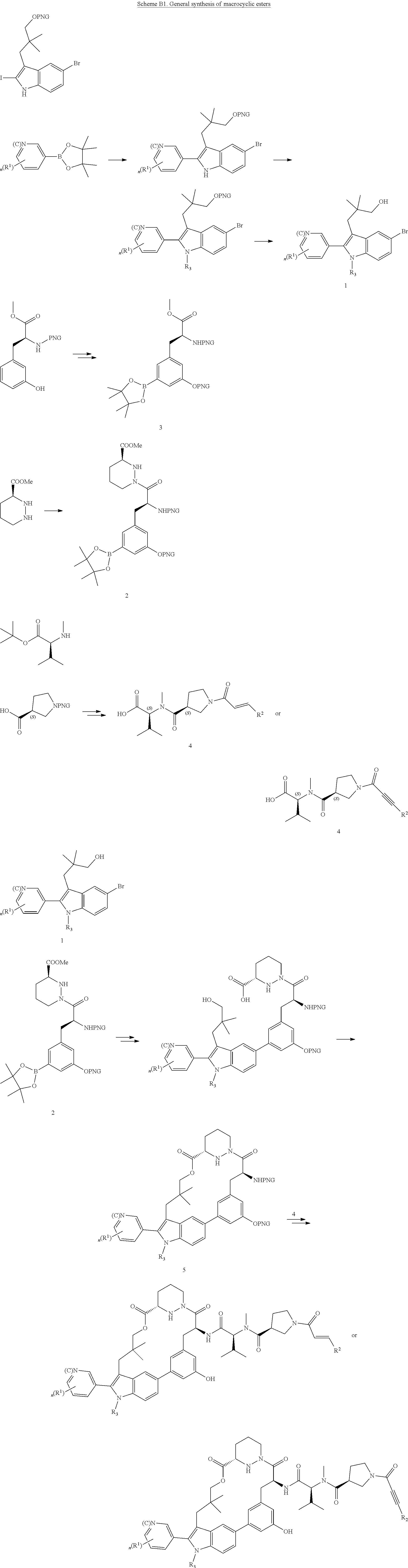

A general synthesis of macrocyclic esters is outlined in Scheme B1. An appropriately substituted aryl-3-(5-bromo-1-ethyl-1H-indol-3-yl)-2,2-dimethylpropan-1-ol (1) can be prepared in three steps starting from protected 3-(5-bromo-2-iodo-1H-indol-3-yl)-2,2-dimethylpropan-1-ol and appropriately substituted boronic acid, including palladium mediated coupling, alkylation, and de-protection reactions.

Methyl-amino-hexahydropyridazine-3-carboxylate-boronic ester (2) can be prepared in three steps, including protection, iridium catalyst mediated borylation, and coupling with methyl methyl (S)-hexahydropyridazine-3-carboxylate.

An appropriately substituted acetylpyrrolidine-3-carbonyl-N-methyl-L-valine (or an alternative aminoacid derivative (4) can be made by coupling of methyl-L-valinate and protected (S)-pyrrolidine-3-carboxylic acid, followed by deprotection, coupling with a carboxylic acid containing an appropriately substituted Michael acceptor, and a hydrolysis step.

The final macrocyclic esters can be made by coupling of methyl-amino-hexahydropyridazine-3-carboxylate-boronic ester (2) and aryl-3-(5-bromo-1-ethyl-1H-indol-3-yl)-2,2-dimethylpropan-1-ol (1) in the presence of a Pd catalyst followed by hydrolysis and macrolactonization steps to result in an appropriately protected macrocyclic intermediate (5). Deprotection and coupling with an appropriately substituted intermediate 4 results in a macrocyclic product. Additional deprotection and/or functionalization steps can be required to produce the final compound.

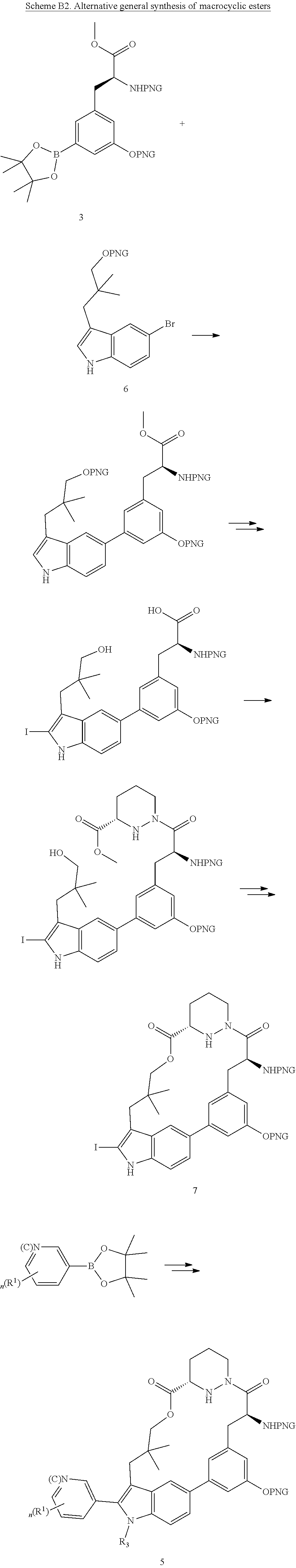

Alternatively, macrocyclic ester can be prepared as described in Scheme B2. An appropriately protected bromo-indolyl (6) coupled in the presence of a Pd catalyst with boronic ester (3), followed by iodination, deprotection, and ester hydrolysis. Subsequent coupling with methyl (S)-hexahydropyridazine-3-carboxylate, followed by hydrolysis and macrolactonization can result in iodo intermediate (7). Coupling in the presence of a Pd catalyst with an appropriately substituted boronic ester and alkylation can yield fully protected macrocycle (5). Additional deprotection or functionalization steps are required to produce the final compound.

In addition, compounds of the disclosure can be synthesized using the methods described in the Examples below or as described in WO 2021/091982, together with synthetic methods known in the art of synthetic organic chemistry, or variations thereon as appreciated by those skilled in the art. These methods include but are not limited to those methods described in the Examples below. For example, a person of skill in the art would be able to install into a macrocyclic ester a desired —B-L-W group of a compound of Formula (BI), where B, L and Ware defined herein, including by using methods exemplified in the Example section herein and in WO 2021/091982.

Compounds of Table B1 herein were prepared using methods disclosed herein or were prepared using methods disclosed herein combined with the knowledge of one of skill in the art. Compounds of Table B2 may be prepared using methods disclosed herein or may be prepared using methods disclosed herein combined with the knowledge of one of skill in the art.

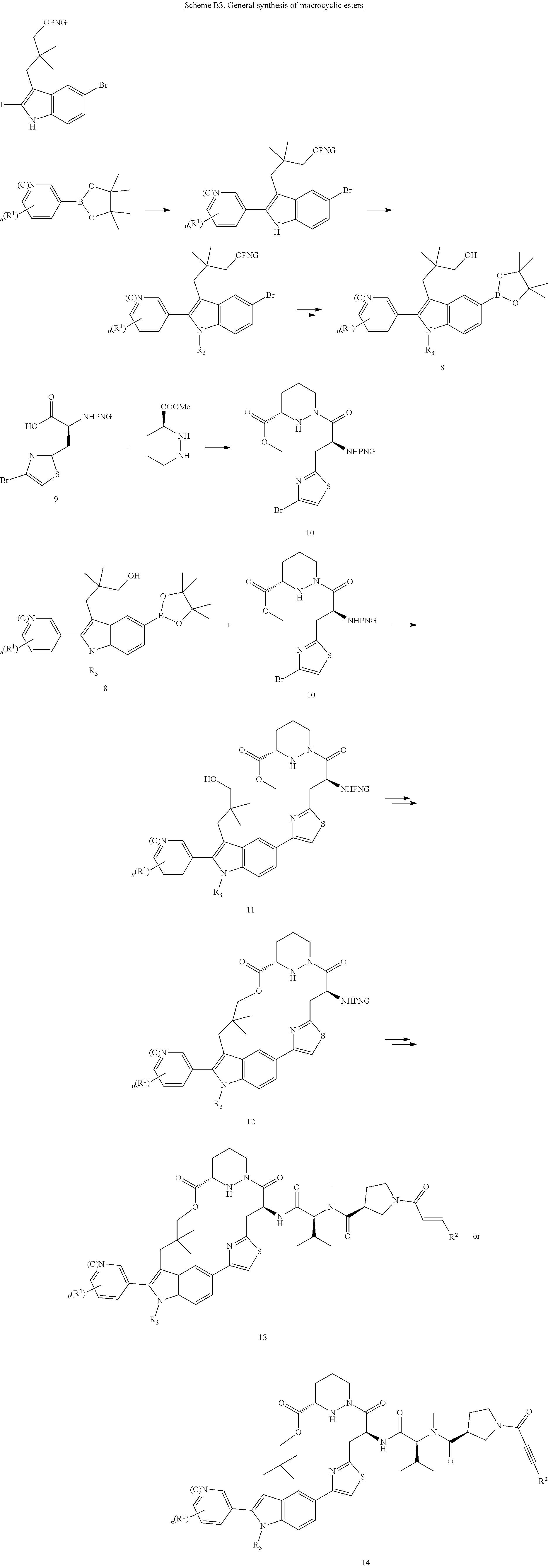

An alternative general synthesis of macrocyclic esters is outlined in Scheme B3. An appropriately substituted indolyl boronic ester (8) can be prepared in four steps starting from protected 3-(5-bromo-2-iodo-1H-indol-3-yl)-2,2-dimethylpropan-1-ol and appropriately substituted boronic acid, including Palladium mediated coupling, alkylation, de-protection, and Palladium mediated borylation reactions.

Methyl-amino-3-(4-bromothiazol-2-yl)propanoyl)hexahydropyridazine-3-carboxylate (10) can be prepared via coupling of (S)-2-amino-3-(4-bromothiazol-2-yl)propanoic acid (9) with methyl (S)-hexahydropyridazine-3-carboxylate.

The final macrocyclic esters can be made by coupling of Methyl-amino-3-(4-bromothiazol-2-yl)propanoyl)hexahydropyridazine-3-carboxylate (10) and an appropriately substituted indolyl boronic ester (8) in the presence of Pd catalyst followed by hydrolysis and macrolactonization steps to result in an appropriately protected macrocyclic intermediate (11). Deprotection and coupling with an appropriately substituted intermediate 4 can result in a macrocyclic product. Additional deprotection or functionalization steps could be required to produce a final compound 13 or 14.

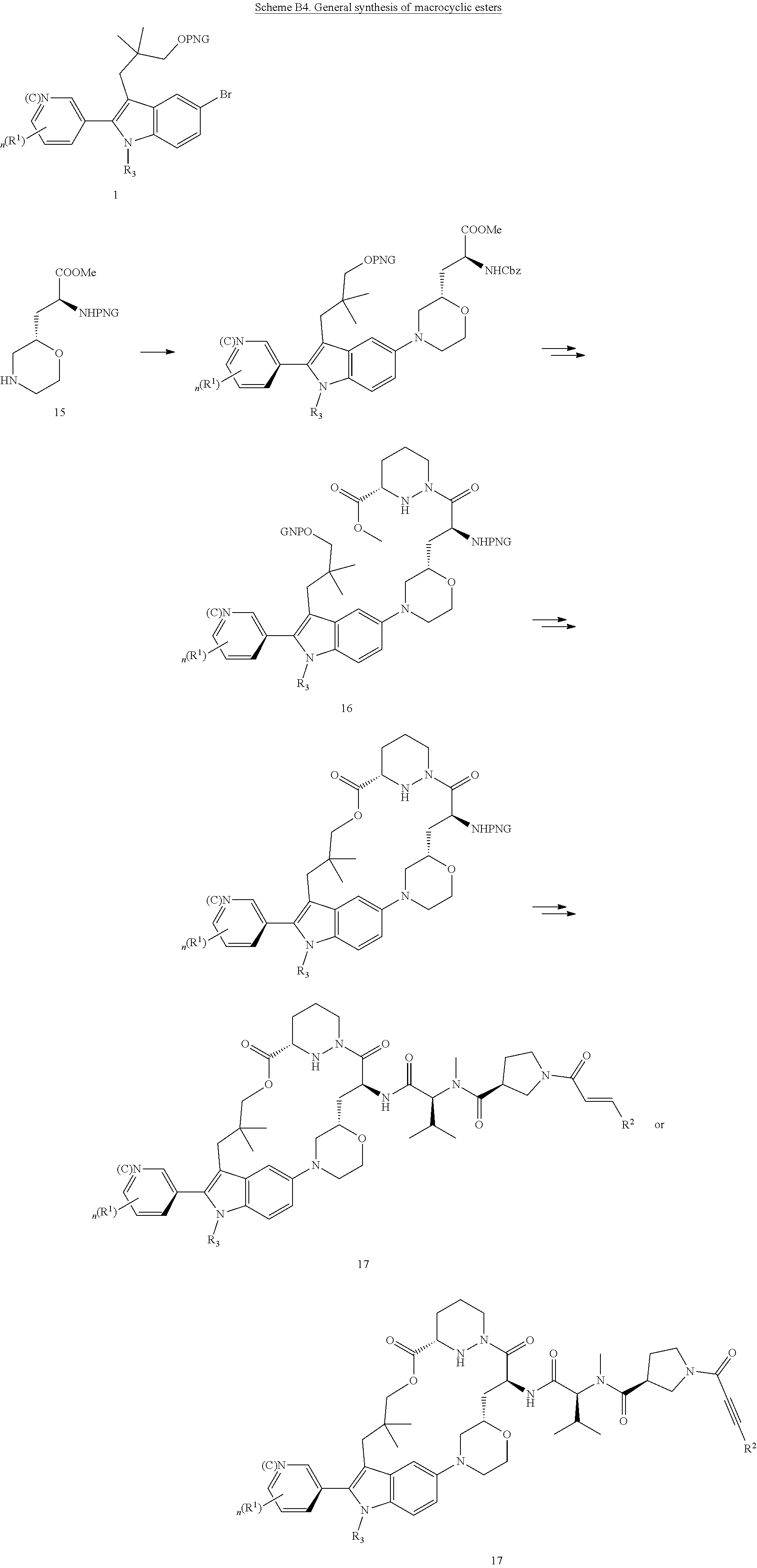

An alternative general synthesis of macrocyclic esters is outlined in Scheme B4. An appropriately substituted morpholine or an alternative heterocyclic intermediate (15) can be coupled with appropriately protected Intermediate 1 via Palladium mediated coupling. Subsequent ester hydrolysis, and coupling with piperazoic ester results in intermediate 16.

The macrocyclic esters can be made by hydrolysis, deprotection and macrocyclization sequence. Subsequent deprotection and coupling with Intermediate 4 (or analogs) result in an appropriately substituted final macrocyclic products. Additional deprotection or functionalization steps could be required to produce a final compound 17.

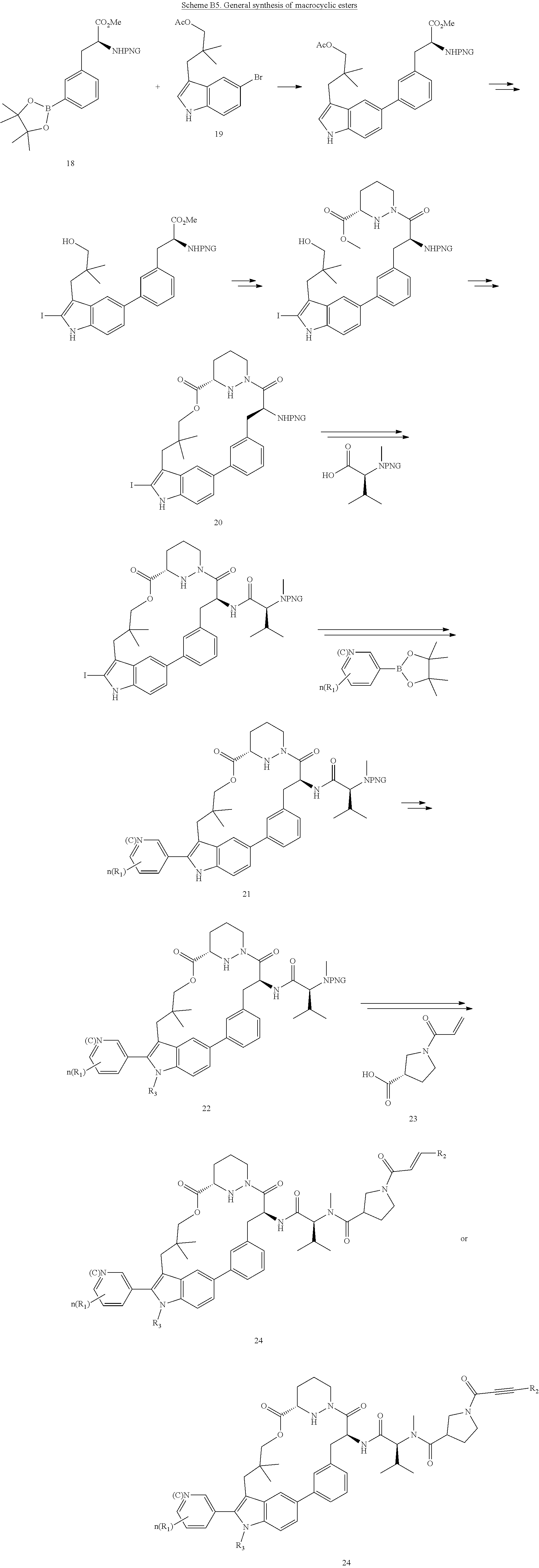

An alternative general synthesis of macrocyclic esters is outlined in Scheme B5. An appropriately substituted macrocycle (20) can be prepared starting from an appropriately protected boronic ester 18 and bromo indolyl intermediate (19), including Palladium mediated coupling, hydrolysis, coupling with piperazoic ester, hydrolysis, de-protection, and macrocyclization steps. Subsequent coupling with an appropriately substituted protected amino acid followed by palladium mediated coupling yields intermediate 21. Additional deprotection and derivatization steps, including alkylation may be required at this point.

The final macrocyclic esters can be made by coupling of intermediate (22) and an appropriately substituted carboxylic acid intermediate (23). Additional deprotection or functionalization steps could be required to produce a final compound (24).

In addition, compounds of the disclosure can be synthesized using the methods described in the Examples below and in WO 2021/091982, together with synthetic methods known in the art of synthetic organic chemistry, or variations thereon as appreciated by those skilled in the art. These methods include but are not limited to those methods described in the Examples below. For example, a person of skill in the art would be able to install into a macrocyclic ester a desired —B-L-W group of a compound of Formula (BI), where B, L and Ware defined herein, including by using methods exemplified in the WO 2021/091982.

In some embodiments, the RAS inhibitor is selective for RAS that includes a G12C amino acid substitution relative to wild-type RAS or other RAS mutants. In some embodiments, the RAS inhibitor is a KRAS inhibitor that is selective for KRAS that includes a G12C amino acid substitution relative to wild-type KRAS or other KRAS mutants. In some embodiments, the RAS inhibitor is an NRAS inhibitor that is selective for NRAS that includes a G12C amino acid substitution relative to wild-type NRAS or other NRAS mutants. In some embodiments, the RAS inhibitor as an HRAS inhibitor that is selective for HRAS that includes a G12C amino acid substitution. In some embodiments, the HRAS inhibitor is selective for HRAS that includes a G12C amino acid substitution relative to wild-type NRAS or other NRAS mutants.

In some embodiments of the combination therapies described herein, the second RAS inhibitor is a RAS(MULTI) inhibitor (e.g., a compound of Formula DIa or a subformula thereof, or a compound of Table D1a, Table D1b, Table 2, or Table 3). RAS(MULTI) inhibitors of Formula DIa are described in WO 2022/060836, the disclosure of which is incorporated herein by reference in its entirety. In some embodiments, the RAS inhibitor is a compound, or a pharmaceutically acceptable salt thereof, having the structure of Formula DIa:

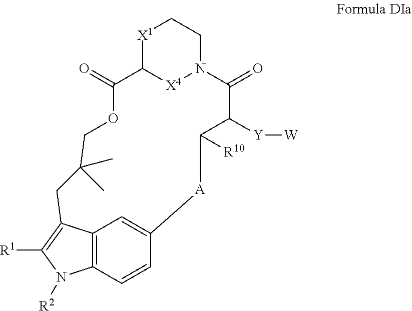

wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
Y is

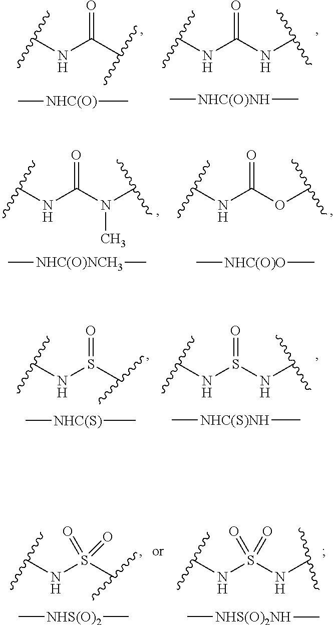

W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X¹ and X⁴ are each, independently, CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and R¹⁰ is hydrogen, hydroxy, optionally substituted C₁-C₃ alkyl, or optionally substituted C₁-C₆ heteroalkyl.

In some embodiments, the RAS inhibitor, or pharmaceutically acceptable salt thereof, has the structure of Formula DIa-2:

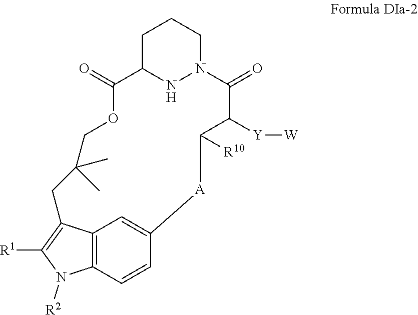

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
Y is

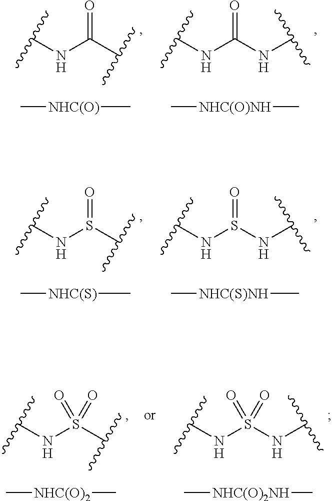

W is hydrogen, C₁-C₄ alkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
R¹⁰ is hydrogen or optionally substituted C₁-C₆ heteroalkyl. In some embodiments, R¹⁰ is hydrogen.

In some embodiments of Formula DIa and subformula thereof, R¹ is optionally substituted 6 to 10-membered aryl or optionally substituted 5 to 10-membered heteroaryl. In some embodiments, R¹ is optionally substituted phenyl or optionally substituted pyridine.

In some embodiments of Formula DIa and subformula thereof, A is optionally substituted thiazole, optionally substituted triazole, optionally substituted morpholino, optionally substituted piperidinyl, optionally substituted pyridine, or optionally substituted phenyl. In some embodiments, A is optionally substituted thiazole, optionally substituted triazole, optionally substituted morpholino, or phenyl. In some embodiments, A is not an optionally substituted phenyl or benzimidazole. In some embodiments, A is not hydroxyphenyl.

In some embodiments of Formula DIa and subformula thereof, Y is —NHC(O)— or —NHC(O)NH—.

In some embodiments, the RAS inhibitor, or a pharmaceutically acceptable salt thereof, has the structure of Formula DIIa:

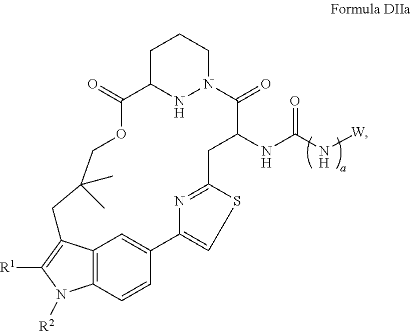

wherein W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
a is 0 or 1.

In some embodiments, the RAS inhibitor, or a pharmaceutically acceptable salt thereof, has the structure of Formula DIIa-1:

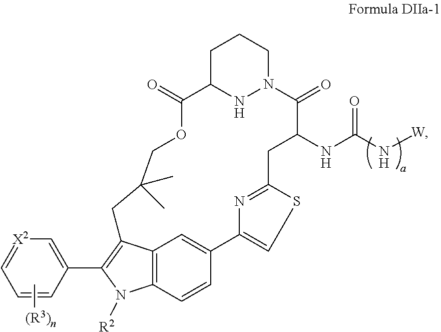

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS inhibitor, or a pharmaceutically acceptable salt thereof, has the structure of Formula DIIa-2:

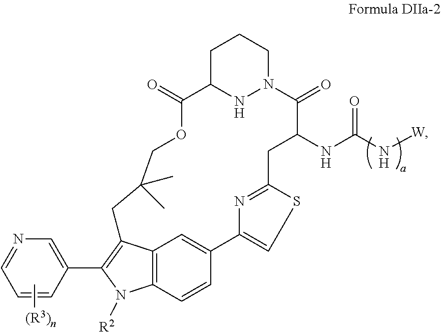

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS inhibitor, or a pharmaceutically acceptable salt thereof, has the structure of Formula DIIa-3:

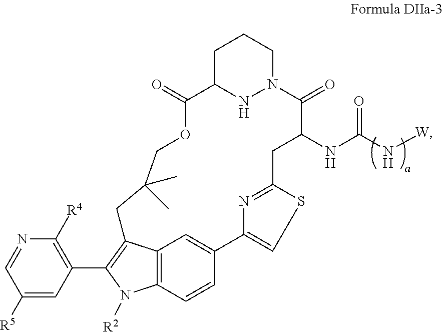

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS inhibitor, or a pharmaceutically acceptable salt thereof, has the structure of Formula DIIa-4:

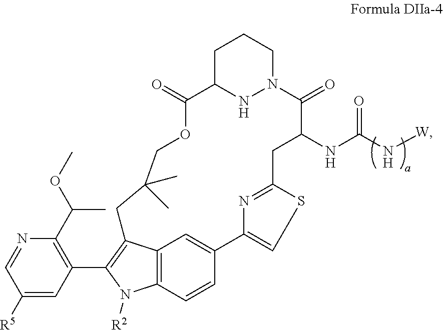

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
R⁵ is halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS inhibitor, or a pharmaceutically acceptable salt thereof, has the structure of Formula DIIa-5:

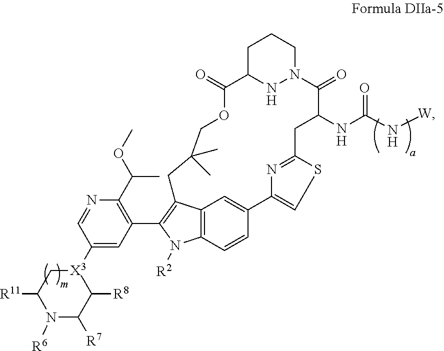

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is H. In some embodiments, X³ is N, m is 1, and R¹¹ is H.

In some embodiments, the RAS inhibitor, or a pharmaceutically acceptable salt thereof, has the structure of Formula DIIa-6:

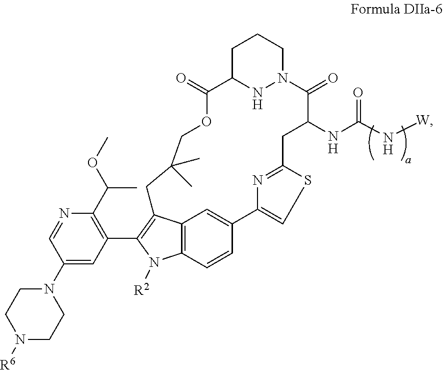

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
R⁶ is hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments, the RAS inhibitor, or a pharmaceutically acceptable salt thereof, has the structure of Formula DIIa-7:

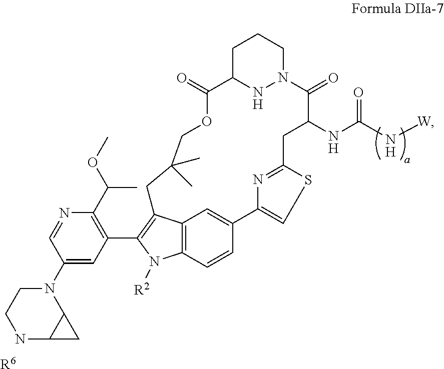

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
R⁶ is hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments (e.g., of any one of Formulae DIIa-6 or DIIa-7), R⁶ is methyl.

In some embodiments, the RAS inhibitor, or a pharmaceutically acceptable salt thereof, has the structure of Formula DIIa-8 or Formula DIIa-9:

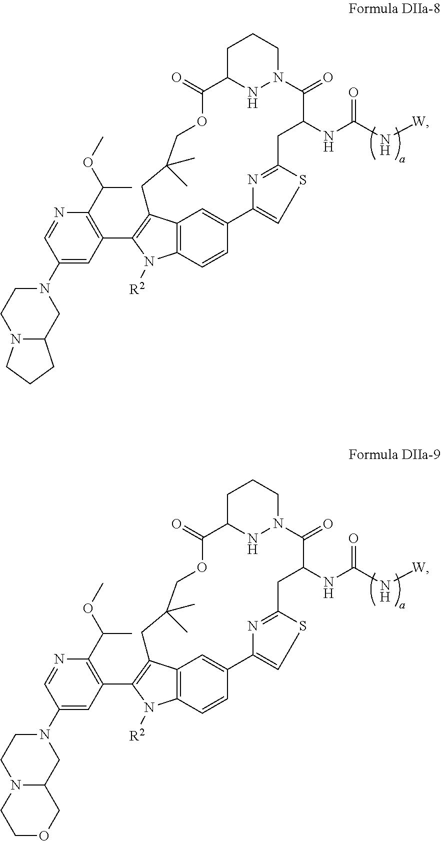

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl.

In some embodiments, the RAS inhibitor, or a pharmaceutically acceptable salt thereof, has the structure of Formula DIIIa:

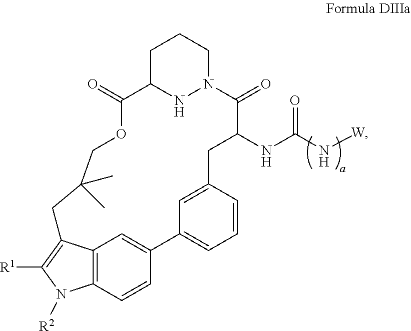

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl.

In some embodiments, the RAS inhibitor, or a pharmaceutically acceptable salt thereof, has the structure of Formula DIIIa-1:

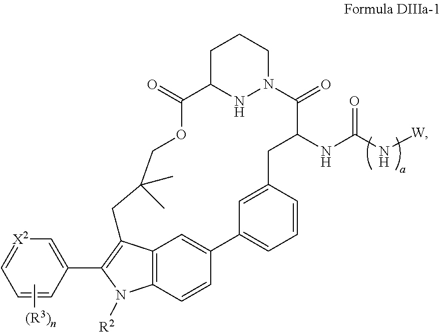

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS inhibitor, or a pharmaceutically acceptable salt thereof, has the structure of Formula DIIIa-2:

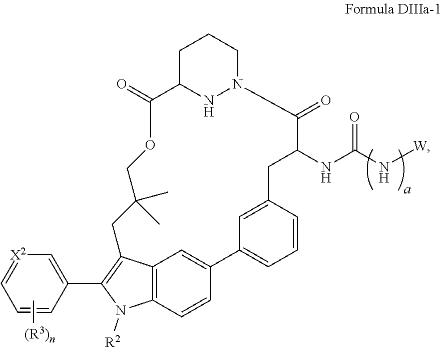

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS inhibitor, or a pharmaceutically acceptable salt thereof, has the structure of Formula DIIIa-3:

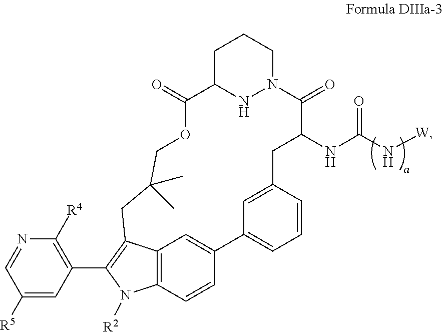

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl;
R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS inhibitor, or a pharmaceutically acceptable salt thereof, has the structure of Formula DIIIa-4:

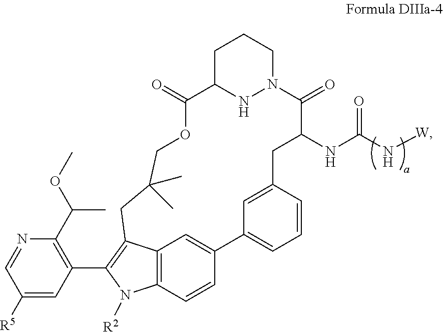

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl; and
R⁵ is halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS inhibitor, or a pharmaceutically acceptable salt thereof, has the structure of Formula DIIIa-5:

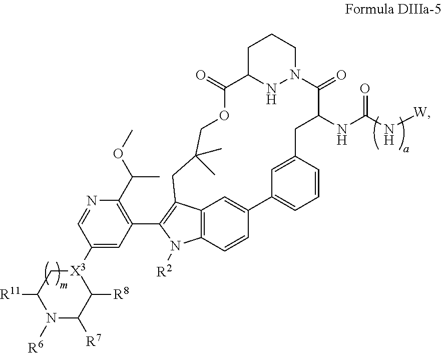

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl;
X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS inhibitor, or a pharmaceutically acceptable salt thereof, has the structure of Formula DIIIa-6:

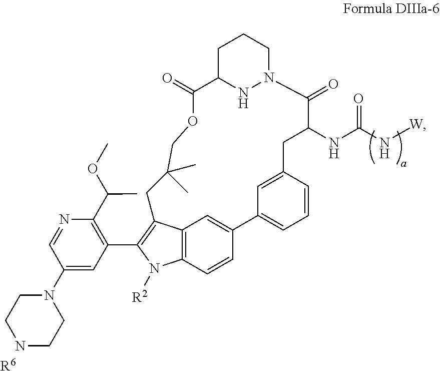

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl; and
R⁶ is hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments, the RAS inhibitor, or a pharmaceutically acceptable salt thereof, has the structure of Formula DIIIa-7:

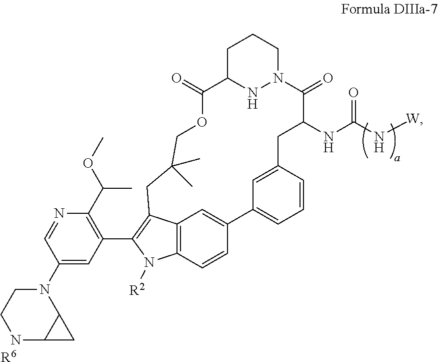

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl; and
R⁶ is hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments (e.g., of any one of Formulae DIIIa-6 or DIIIa-7), R⁶ is methyl.

In some embodiments, the RAS inhibitor, or a pharmaceutically acceptable salt thereof, has the structure of Formula DIIIa-8 or Formula DIIIa-9:

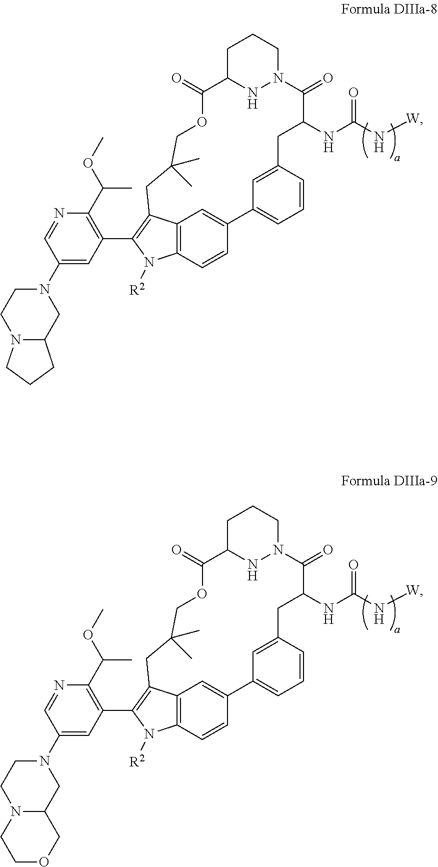

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl.

In some embodiments, the RAS inhibitor, or a pharmaceutically acceptable salt thereof, has the structure of Formula DIVa:

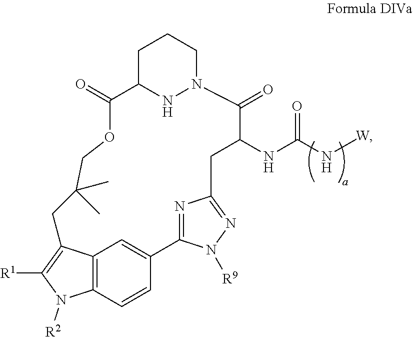

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl; and
a is 0 or 1.

In some embodiments, the RAS inhibitor, or a pharmaceutically acceptable salt thereof, has the structure of Formula DIVa-1:

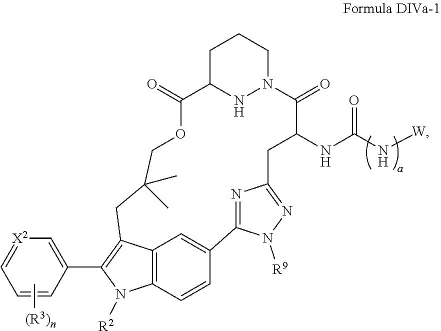

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl;
R⁹ is H or C₁-C₆ alkyl; and
n is an integer from 1 to 4.

In some embodiments, the RAS inhibitor, or a pharmaceutically acceptable salt thereof, has the structure of Formula DIVa-2:

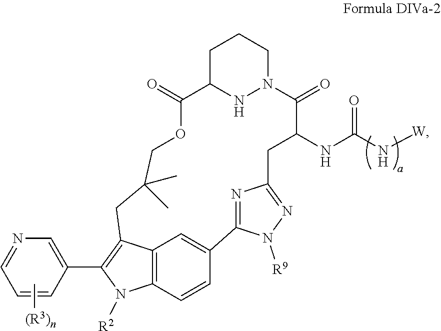

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl;
R⁹ is H or C₁-C₆ alkyl; and
n is an integer from 1 to 4.

In some embodiments, the RAS inhibitor, or a pharmaceutically acceptable salt thereof, has the structure of Formula DIVa-3:

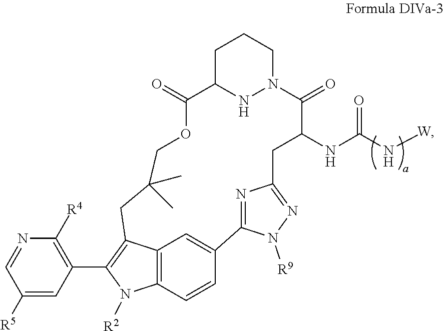

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R⁹ is H or C₁-C₆ alkyl; and
R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS inhibitor, or a pharmaceutically acceptable salt thereof, has the structure of Formula DIVa-4:

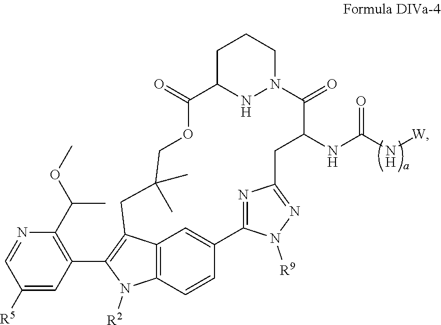

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R⁹ is H or C₁-C₆ alkyl; and
R⁵ is halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS inhibitor, or a pharmaceutically acceptable salt thereof, has the structure of Formula DIVa-5:

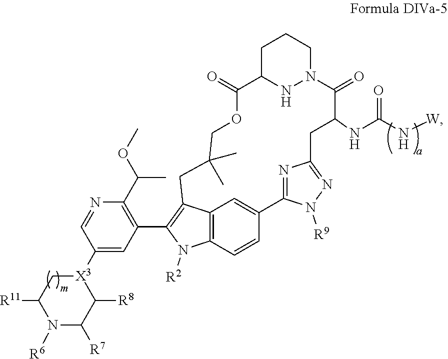

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X³ is N or CH;
m is 1 or 2;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R⁹ is H or C₁-C₆ alkyl;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS inhibitor, or a pharmaceutically acceptable salt thereof, has the structure of Formula DIVa-6:

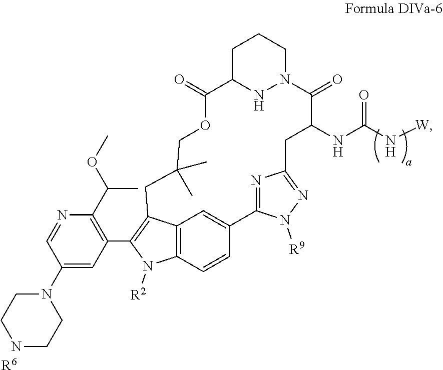

In some embodiments, the RAS inhibitor, or a pharmaceutically acceptable salt thereof, has the structure of Formula DIVa-7:

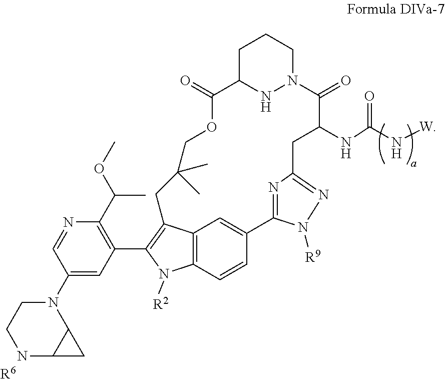

In some embodiments (e.g., of any one of Formulae DIVa-6 or DIVa-7), R⁶ is methyl.

In some embodiments, the RAS inhibitor, or a pharmaceutically acceptable salt thereof, has the structure of Formula DIVa-8 or Formula DIVa-9:

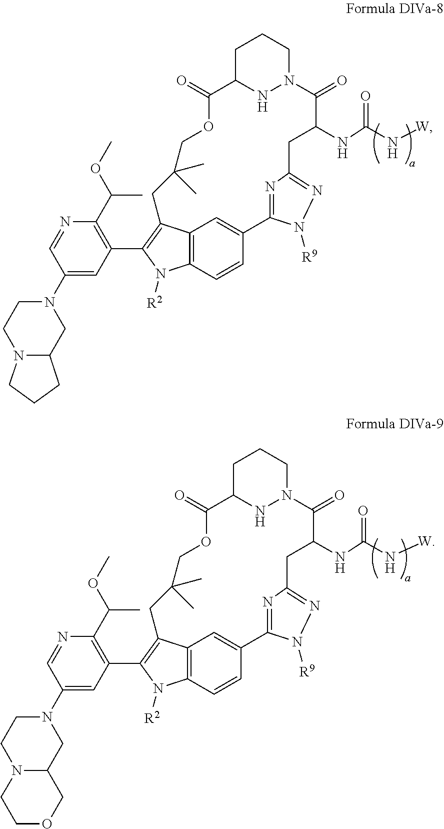

In some embodiments (e.g., of any one of Formulae DIVa, DIVa-1, DIVa-2, DIVa-3, DIVa-4, DIVa-5, DIVa-6, DIVa-7, DIVa-8, or DIVa-9), R⁹ is methyl.

In some embodiments, Y is —NHS(O)₂— or —NHS(O)₂NH—.

In some embodiments, the RAS inhibitor, or a pharmaceutically acceptable salt thereof, has the structure of Formula DVa:

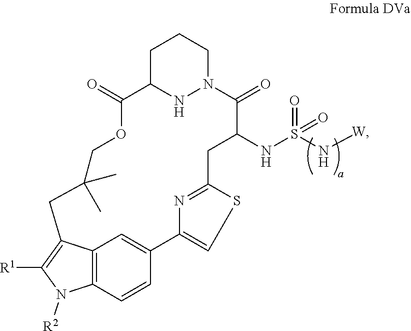

wherein a is 0 or 1.

In some embodiments, the RAS inhibitor, or a pharmaceutically acceptable salt thereof, has the structure of Formula DVa-1:

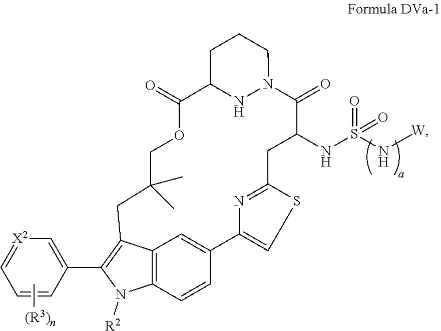

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS inhibitor, or a pharmaceutically acceptable salt thereof, has the structure of Formula DVa-2:

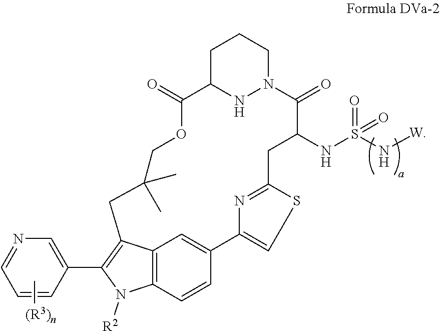

In some embodiments, the RAS inhibitor, or a pharmaceutically acceptable salt thereof, has the structure of Formula DVa-3:

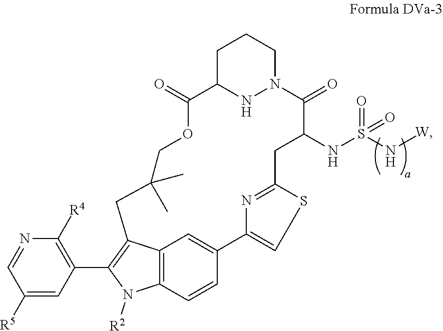

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS inhibitor, or a pharmaceutically acceptable salt thereof, has the structure of Formula DVa-4:

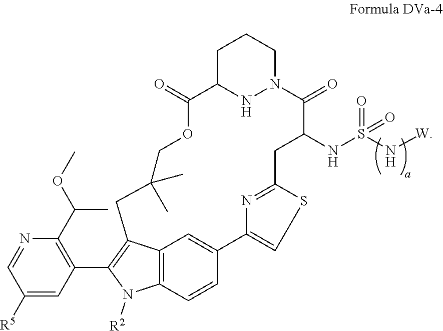

In some embodiments, the RAS inhibitor, or a pharmaceutically acceptable salt thereof, has the structure of Formula DVa-5:

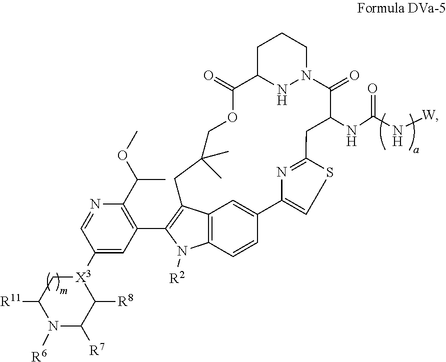

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS inhibitor, or a pharmaceutically acceptable salt thereof, has the structure of Formula DVIa:

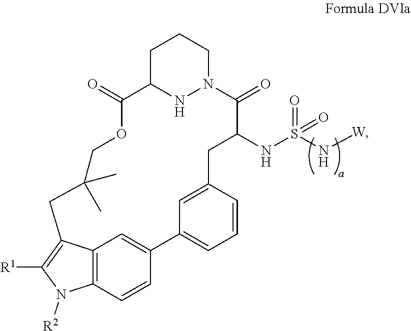

wherein a is 0 or 1.

In some embodiments, the RAS inhibitor, or a pharmaceutically acceptable salt thereof, has the structure of Formula DVIa-1:

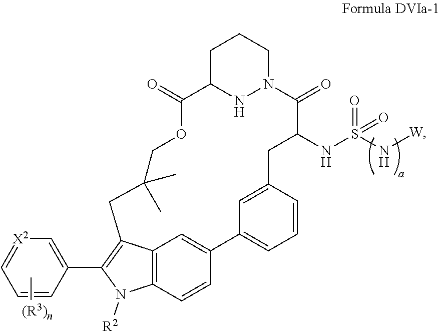

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS inhibitor, or a pharmaceutically acceptable salt thereof, has the structure of Formula DVIa-2:

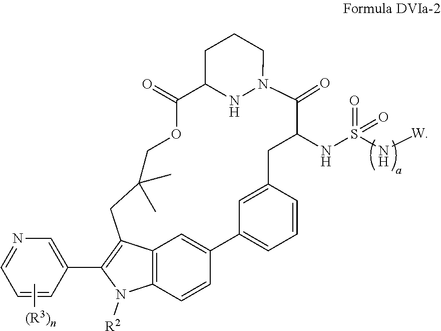

In some embodiments, the RAS inhibitor, or a pharmaceutically acceptable salt thereof, has the structure of Formula DVIa-3:

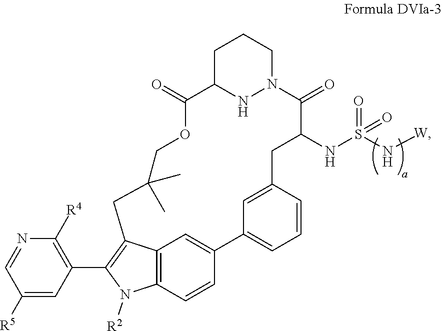

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS inhibitor, or a pharmaceutically acceptable salt thereof, has the structure of Formula DVIa-4:

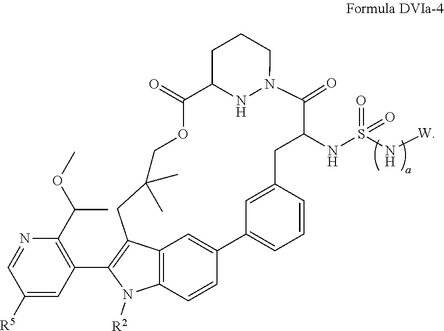

In some embodiments, the RAS inhibitor, or a pharmaceutically acceptable salt thereof, has the structure of Formula DVIa-5:

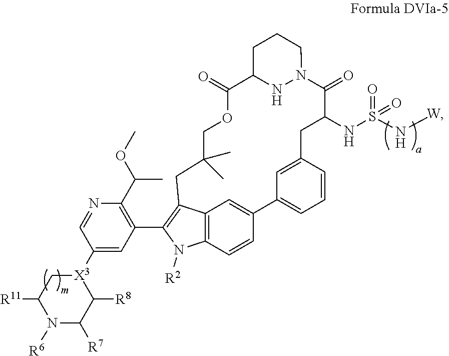

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS inhibitor, or a pharmaceutically acceptable salt thereof, has the structure of Formula DVIIa:

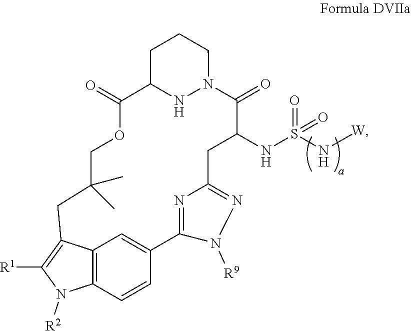

wherein R⁹ is H or C₁-C₆ alkyl; and
a is 0 or 1.

In some embodiments, the RAS inhibitor, or a pharmaceutically acceptable salt thereof, has the structure of Formula DVIIa-1:

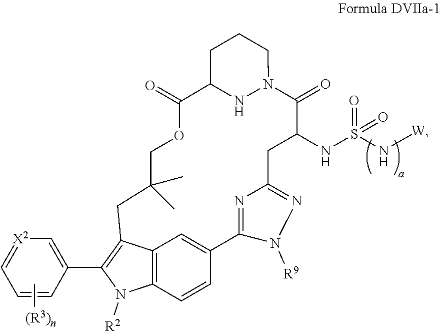

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS inhibitor, or a pharmaceutically acceptable salt thereof, has the structure of Formula DVIIa-2:

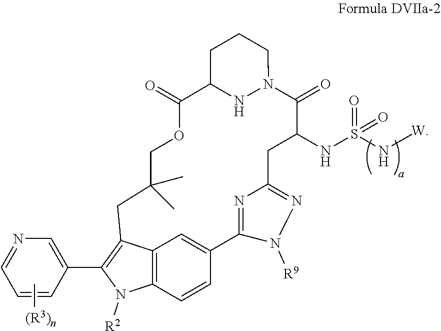

In some embodiments, the RAS inhibitor, or a pharmaceutically acceptable salt thereof, has the structure of Formula DVIIa-3:

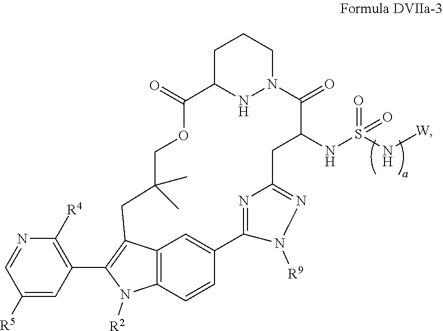

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS inhibitor, or a pharmaceutically acceptable salt thereof, has the structure of Formula DVIIa-4:

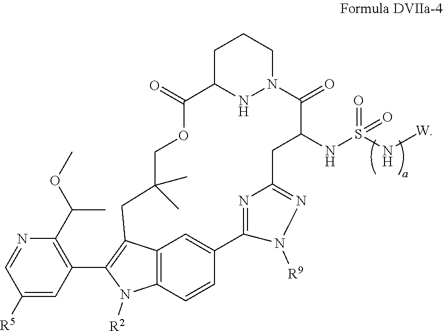

In some embodiments, the RAS inhibitor, or a pharmaceutically acceptable salt thereof, has the structure of Formula DVIIa-5:

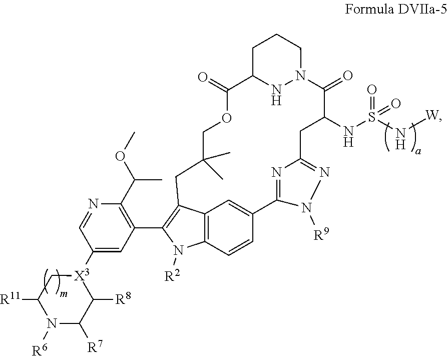

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments (e.g., of any one of Formulae DVIIa, DVIIa-1, DVIIa-2, DVIIa-3, DVIIa-4, or DVIIa-5), R⁹ is methyl.

In some embodiments, Y is —NHS(O)— or —NHS(O)NH—.

In some embodiments, the RAS inhibitor, or a pharmaceutically acceptable salt thereof, has the structure of Formula DVIIIa:

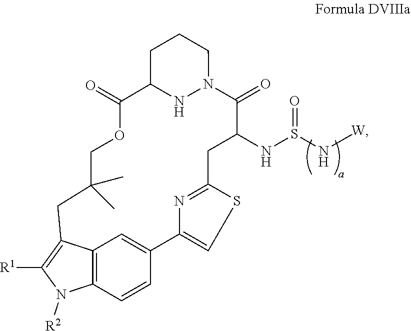

wherein a is 0 or 1.

In some embodiments, the RAS inhibitor, or a pharmaceutically acceptable salt thereof, has the structure of Formula VIIIa-1:

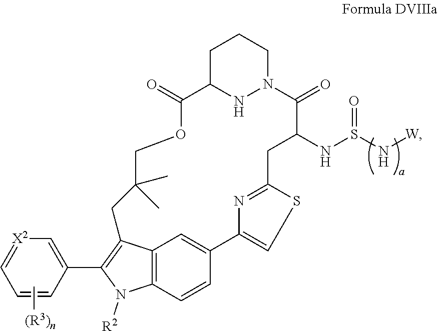

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS inhibitor, or a pharmaceutically acceptable salt thereof, has the structure of Formula DVIIIa-2:

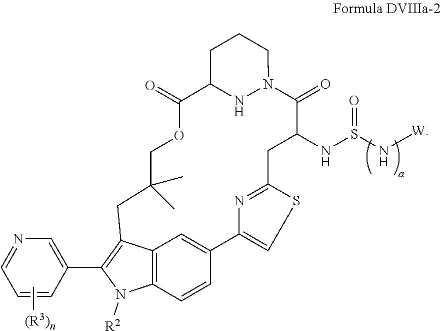

In some embodiments, the RAS inhibitor, or a pharmaceutically acceptable salt thereof, has the structure of Formula DVIIIa-3:

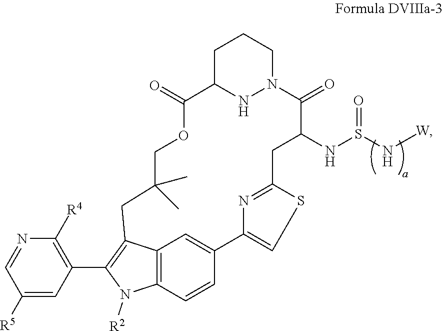

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS inhibitor, or a pharmaceutically acceptable salt thereof, has the structure of Formula DVIIIa-4:

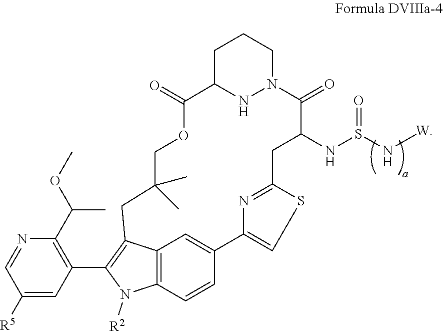

In some embodiments, the RAS inhibitor, or a pharmaceutically acceptable salt thereof, has the structure of Formula DVIIIa-5:

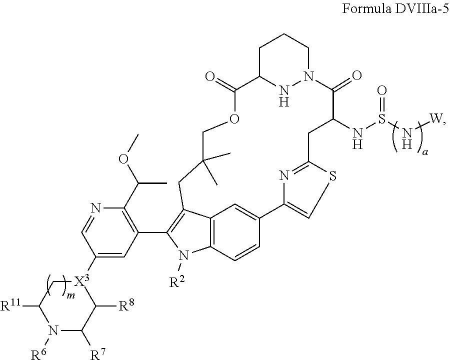

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS inhibitor, or a pharmaceutically acceptable salt thereof, has the structure of Formula DIXa:

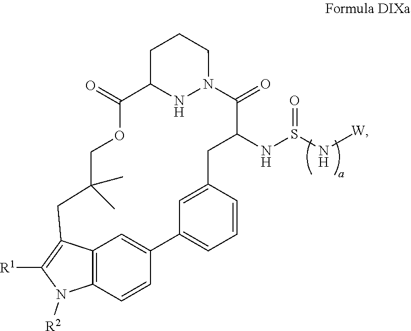

wherein a is 0 or 1.

In some embodiments, the RAS inhibitor, or a pharmaceutically acceptable salt thereof, has the structure of Formula DIXa-1:

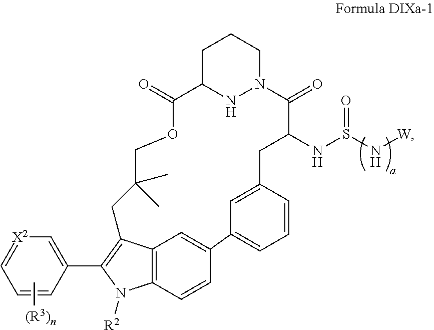

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS inhibitor, or a pharmaceutically acceptable salt thereof, has the structure of Formula DIXa-2:

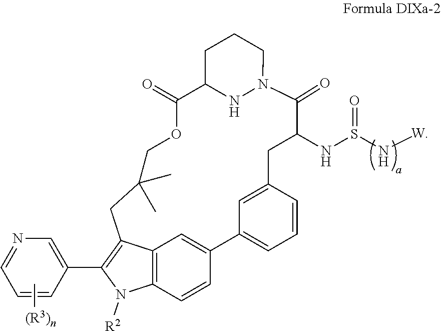

In some embodiments, the RAS inhibitor, or a pharmaceutically acceptable salt thereof, has the structure of Formula DIXa-3:

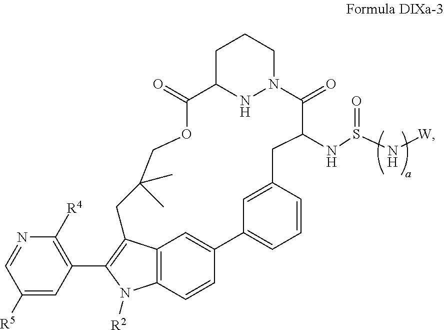

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS inhibitor, or a pharmaceutically acceptable salt thereof, has the structure of Formula DIXa-4:

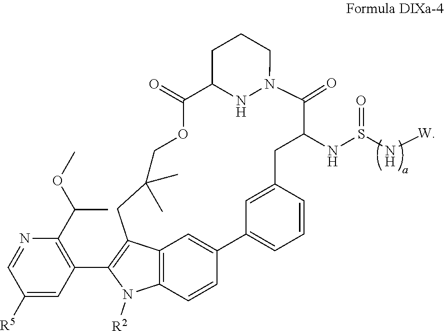

In some embodiments, the RAS inhibitor, or a pharmaceutically acceptable salt thereof, has the structure of Formula DIXa-5:

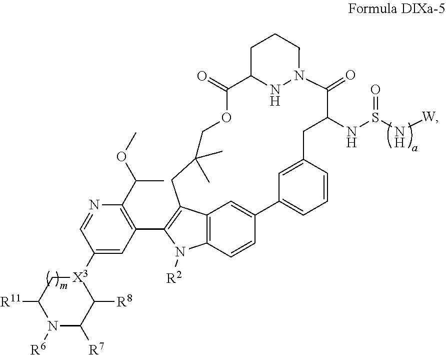

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS inhibitor, or a pharmaceutically acceptable salt thereof, has the structure of Formula DXa:

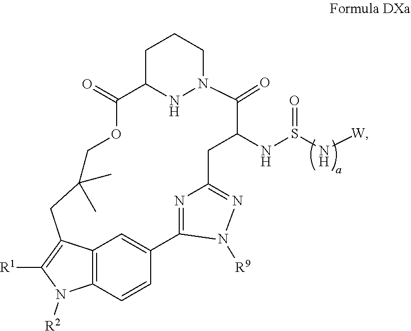

wherein R⁹ is H or C₁-C₆ alkyl; and
a is 0 or 1.

In some embodiments, the RAS inhibitor, or a pharmaceutically acceptable salt thereof, has the structure of Formula DXa-1:

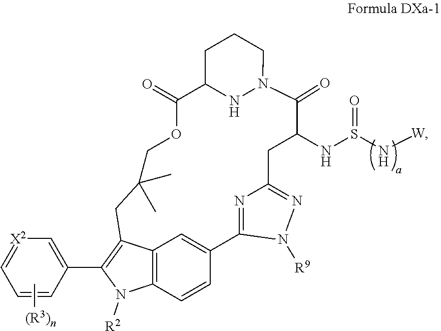

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS inhibitor, or a pharmaceutically acceptable salt thereof, has the structure of Formula DXa-2:

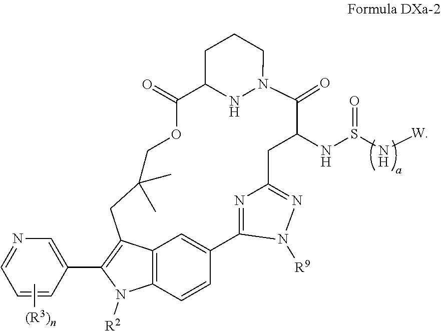

In some embodiments, the RAS inhibitor, or a pharmaceutically acceptable salt thereof, has the structure of Formula DXa-3:

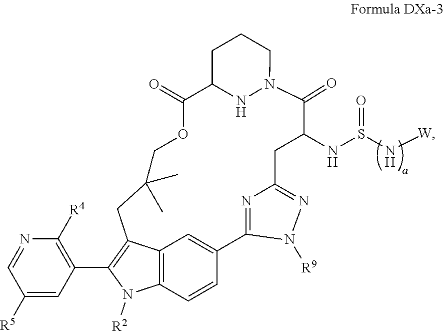

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS inhibitor, or a pharmaceutically acceptable salt thereof, has the structure of Formula DXa-4:

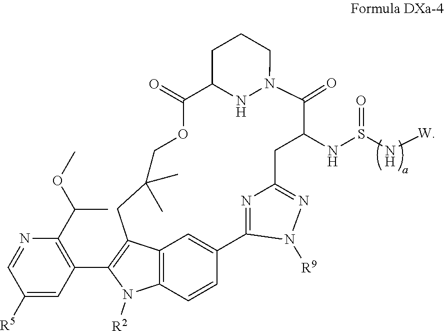

In some embodiments, the RAS inhibitor, or a pharmaceutically acceptable salt thereof, has the structure of Formula DXa-5:

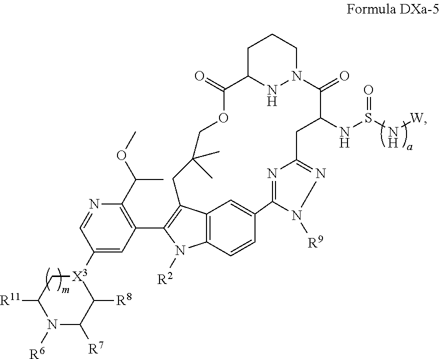

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments (e.g., of any one of Formulae DXa, DXa-1, DXa-2, DXa-3, DXa-4, or DXa-5), R⁹ is methyl.

In some embodiments of formula DIa or subformula thereof, a is 0. In some embodiments of formula DIa or subformula thereof, a is 0.

In some embodiments of formula DIa or subformula thereof, R² is optionally substituted C₁-C₆ alkyl. In some embodiments, R² is selected from —CH₂CH₃ or —CH₂CF₃.

In some embodiments of formula DIa or subformula thereof, W is C₁-C₄ alkyl. In some embodiments, W is:

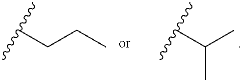

In some embodiments of formula DIa or subformula thereof, W is optionally substituted cyclopropyl, optionally substituted cyclobutyl, optionally substituted cyclopentyl, or optionally substituted cyclohexyl, optionally substituted piperidine, optionally substituted piperazine, optionally substituted pyridine, or optionally substituted phenyl.

In some embodiments of formula DIa or subformula thereof, W is optionally substituted 3 to 10-membered heterocycloalkyl, optionally substituted 3 to 10-membered cycloalkyl, optionally substituted 6 to 10-membered aryl, or optionally substituted 5 to 10-membered heteroaryl.

In some embodiments of formula DIa or subformula thereof, W is optionally substituted 3 to 10-membered heterocycloalkyl. In some embodiments, W is selected from the following, or a stereoisomer thereof:

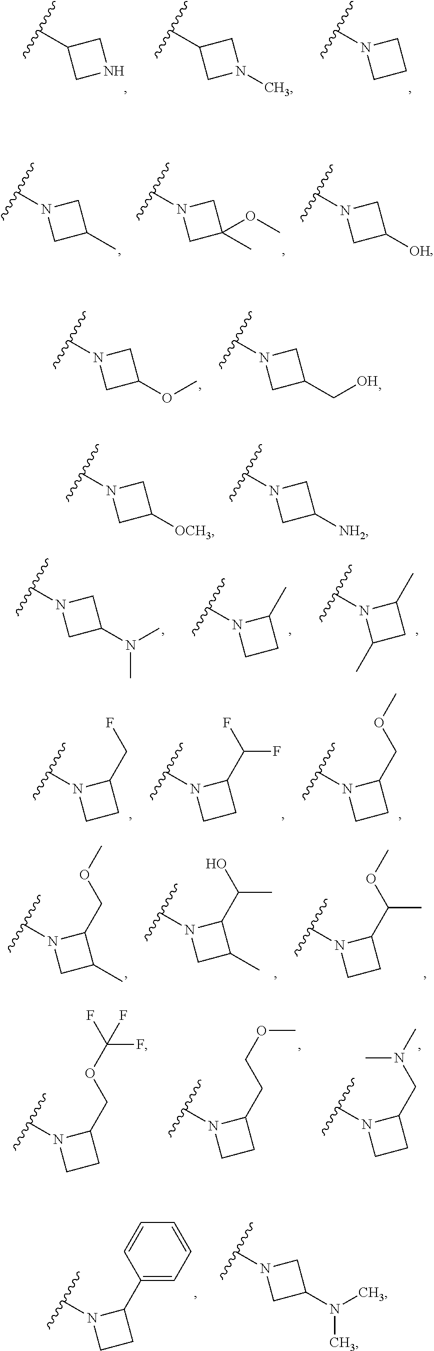

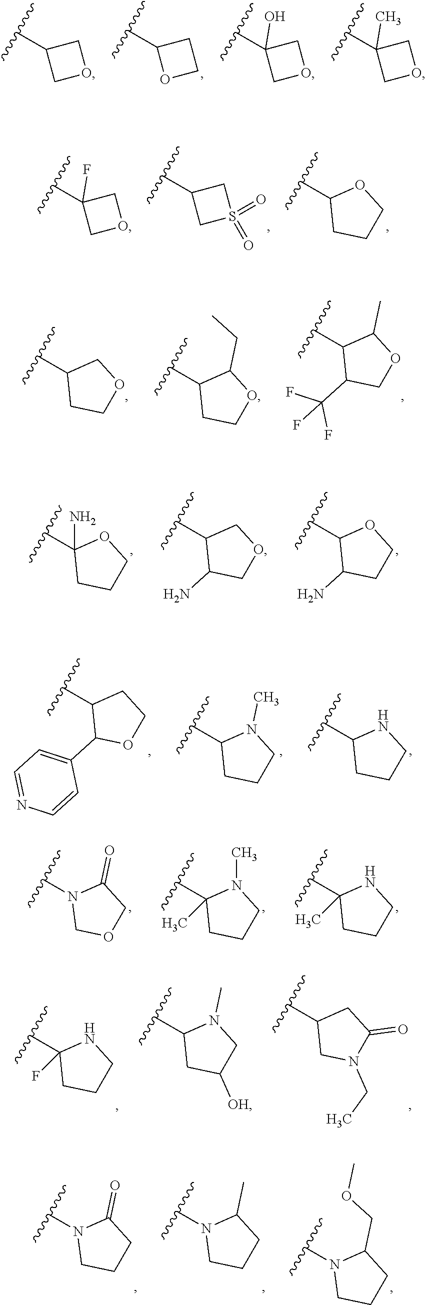

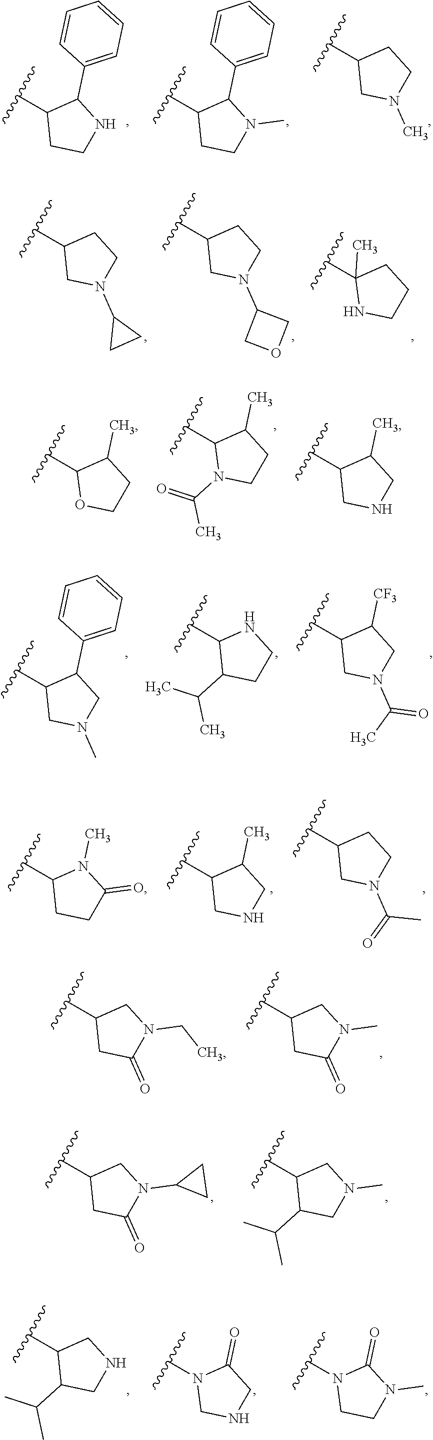

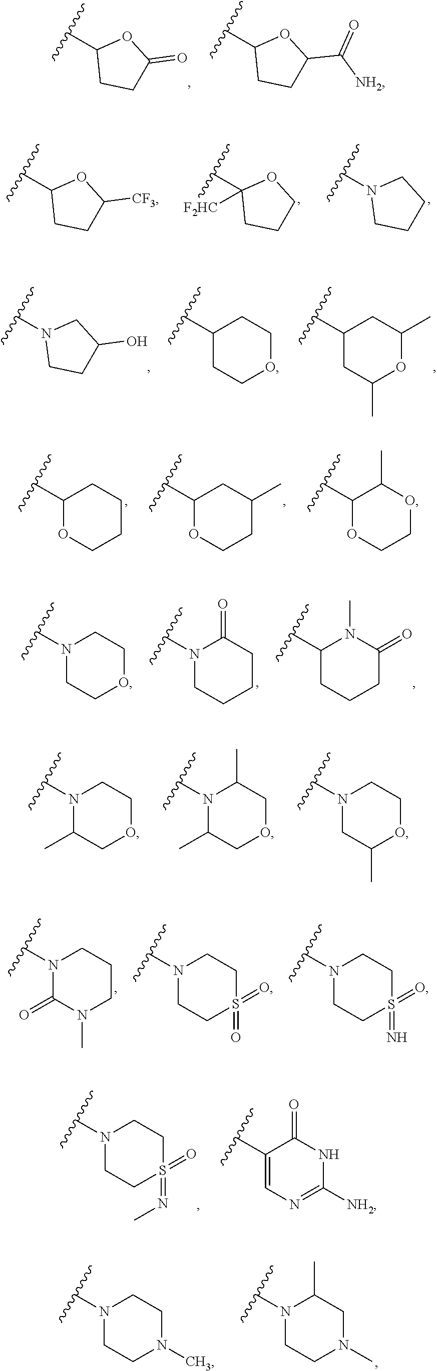

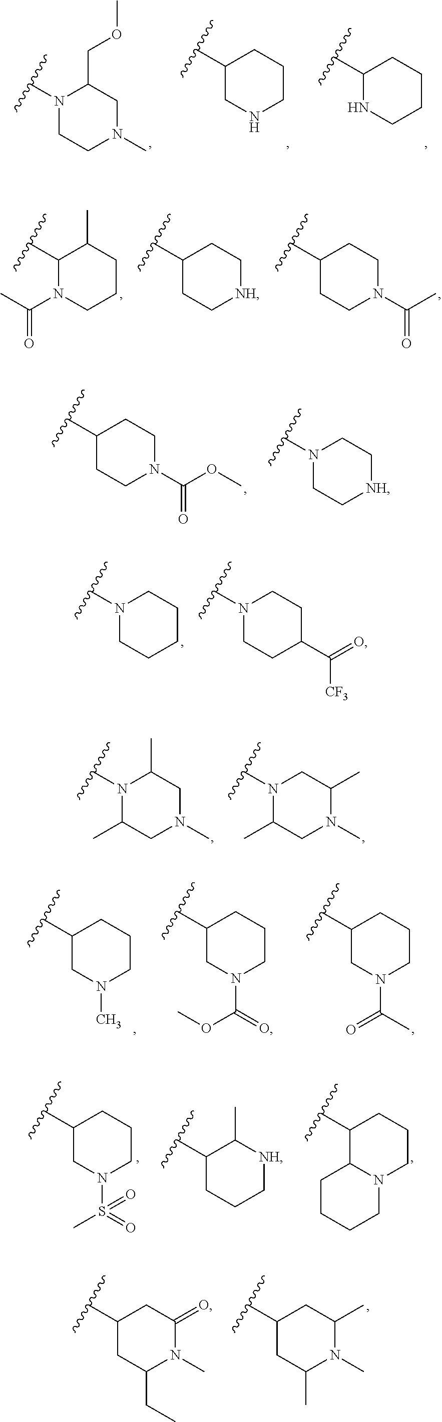

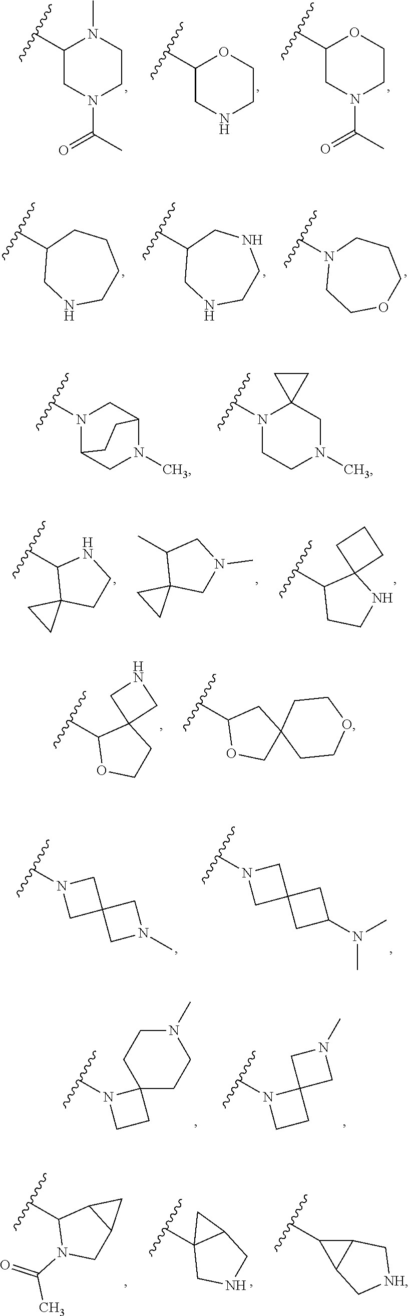

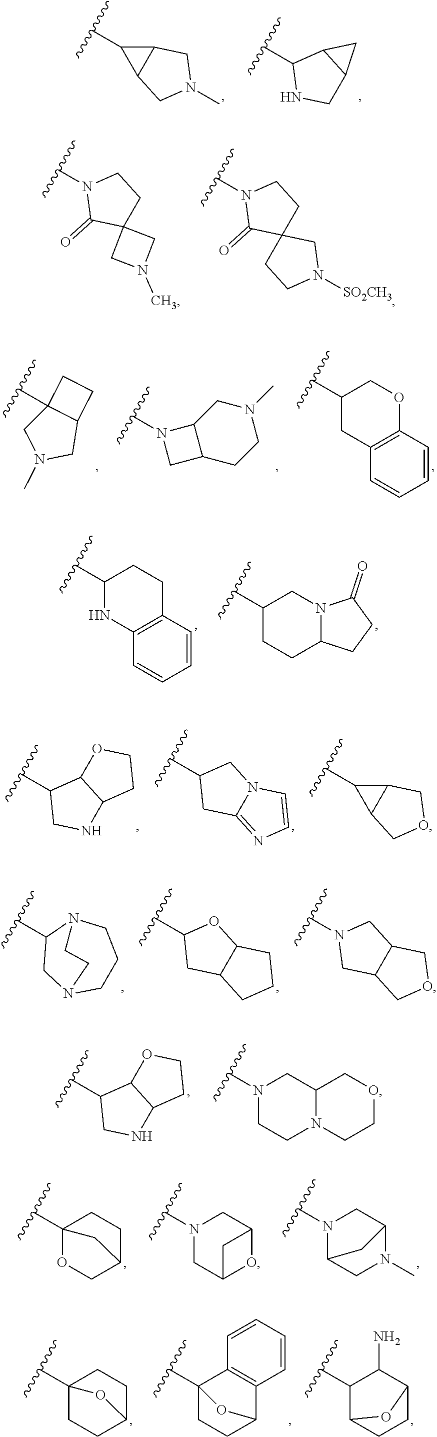

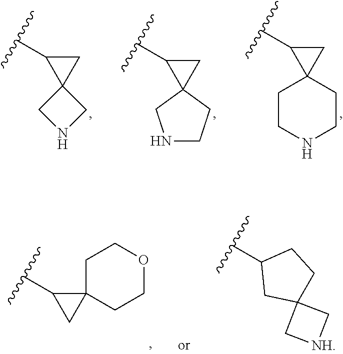 [SMILES: *C1CC12CNC2 ; *C1CC12CCNC2 ; *C1CC12CCNCC2 ; *C1CC12CCOCC2 ; *C1CCC2(CNC2)C1]

In some embodiments, W is selected from the following, or a stereoisomer thereof:

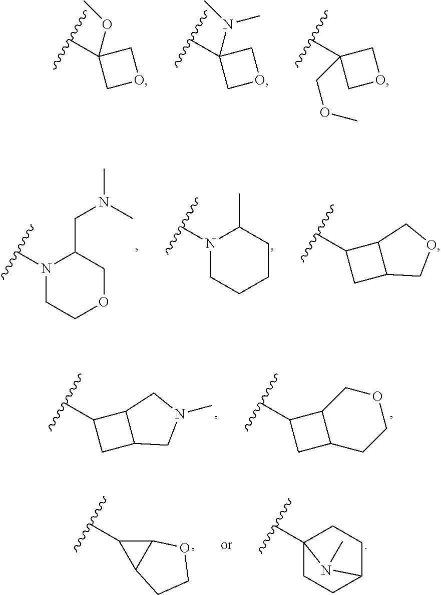

In some embodiments of formula DIa or subformula thereof, W is optionally substituted 3 to 10-membered cycloalkyl. In some embodiments, W is selected from the following, or a stereoisomer thereof:

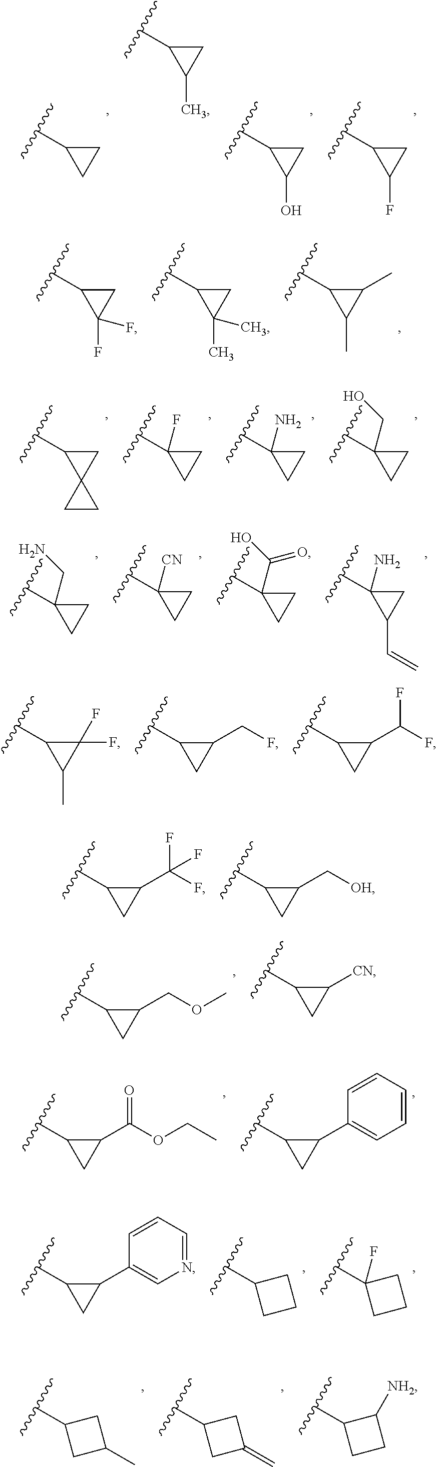

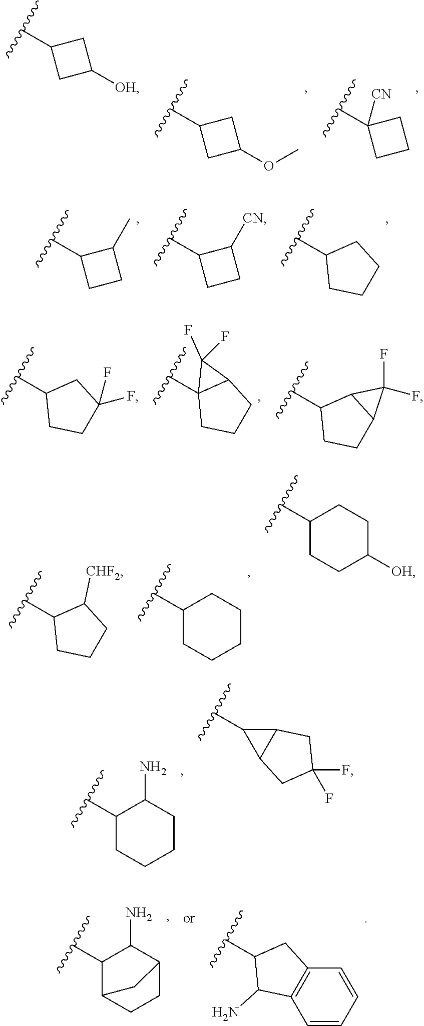

In some embodiments, W is selected from the following, or a stereoisomer thereof:

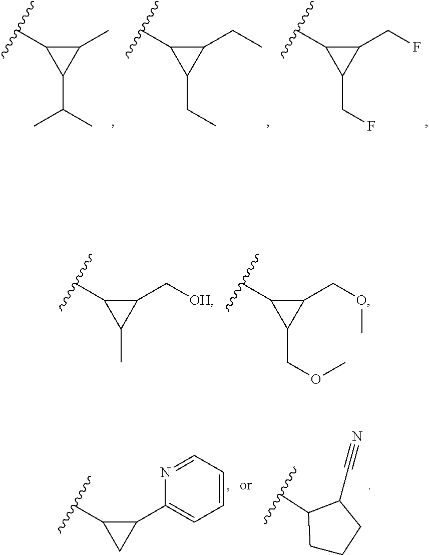

In some embodiments of formula DIa or subformula thereof, W is optionally substituted 5 to 10-membered heteroaryl. In some embodiments, W is selected from the following, or a stereoisomer thereof:

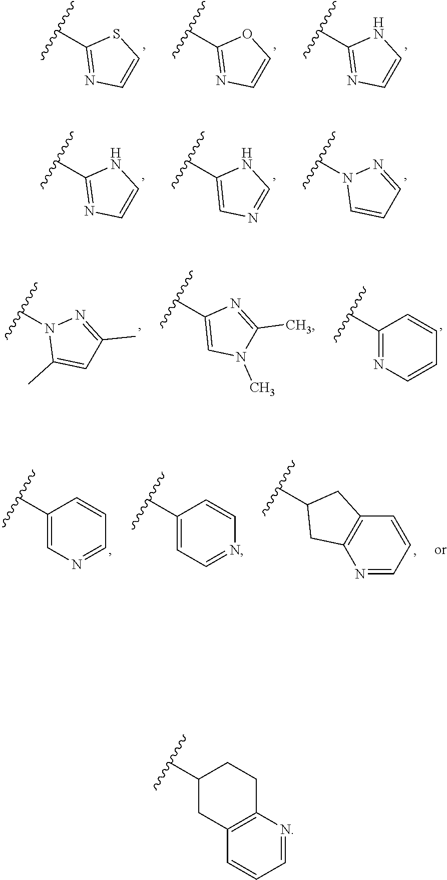

In some embodiments of formula DIa or subformula thereof, W is optionally substituted 6 to 10-membered aryl. In some embodiments, W is optionally substituted phenyl.

In some embodiments of formula DIa or subformula thereof, W is optionally substituted C₁-C₃ heteroalkyl. In some embodiments, W is selected from the following, or a stereoisomer thereof:

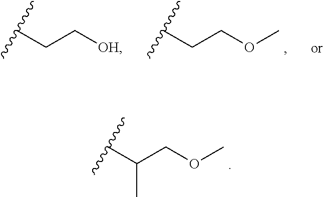

In some embodiments, the RAS inhibitor, or pharmaceutically acceptable salt thereof, has the structure of Formula DIb:

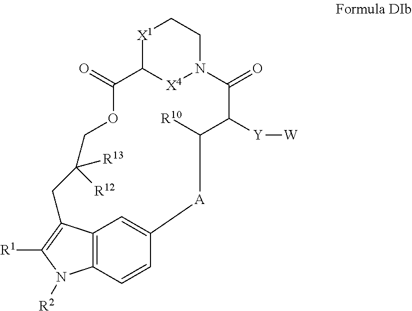

wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
Y is

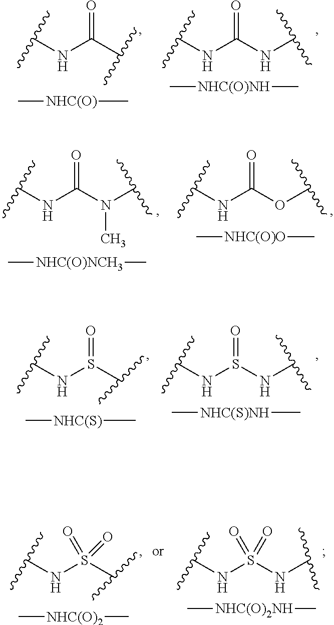

W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl; or W is —R¹⁴C(═O)R¹⁵ where R¹⁴ is 3 to 10-membered cycloalkylene and R¹⁵ is selected from optionally substituted 3 to 10-membered cycloalkyl, optionally substituted 6 to 10-membered aryl, or optionally substituted 5 to 10-membered heteroaryl;
X¹ and X⁴ are each, independently, CH₂, CH(CH₃) or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R¹⁰ is hydrogen, hydroxy, optionally substituted C₁-C₆ alkoxy, optionally substituted C₁-C₃ alkyl, optionally substituted C₁-C₆ heteroalkyl, or optionally substituted 3 to 7-membered heterocycloalkyl; and
R¹² and R¹³ are each, independently, selected from F or CH₃, or R¹² and R¹³ combine with the atoms to which they are attached to make a 3-membered cycloalkyl

In some embodiments, the RAS inhibitor is a RAS(MULTI) inhibitor. In some embodiments, the RAS(MULTI) inhibitor is selected from Table D1a, or a pharmaceutically acceptable salt or stereoisomer thereof. In some embodiments, the RAS(MULTI) inhibitor is selected from Table D1a, or a pharmaceutically acceptable salt or atropisomer thereof.

TABLE D1a
Certain Compounds of the Present Invention

| Ex. | |
|---|---|
| # | Structure |
| DA 1 | 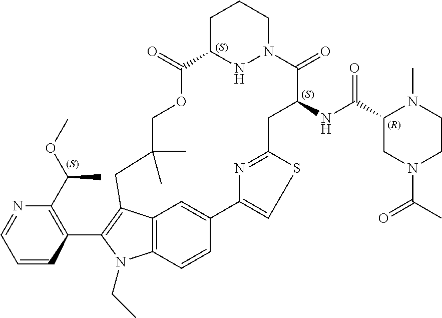 |
| DA 2 | 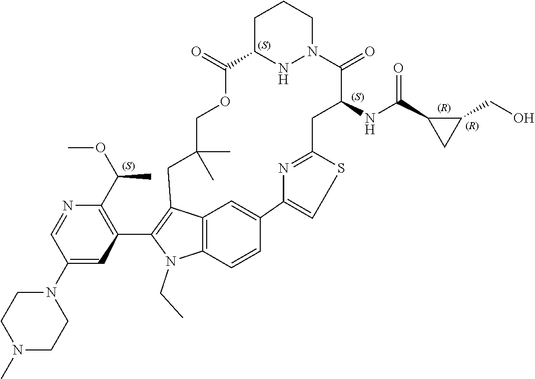 |
| DA 3 | 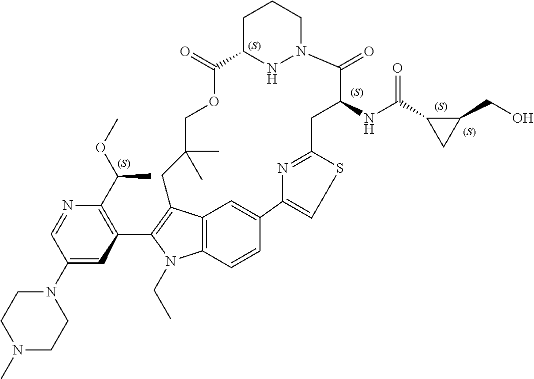 |
| DA 4 | 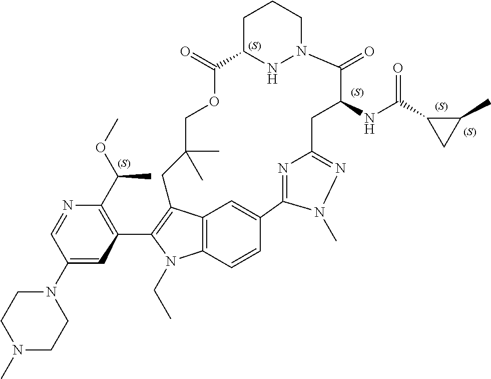 |
| DA 5 | 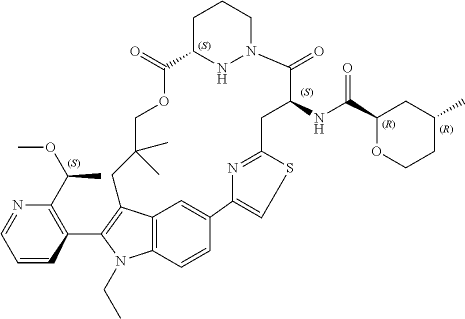 |
| DA 6 | 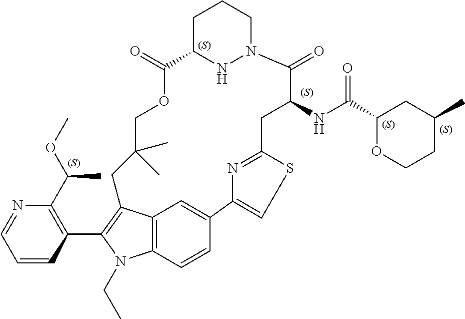 |
| DA 7 | 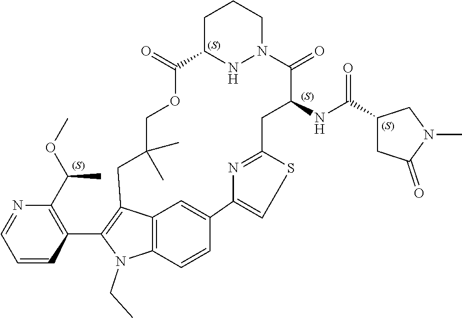 |
| DA 8 | 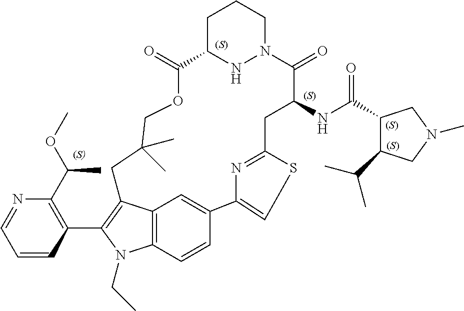 |
| DA 9 | 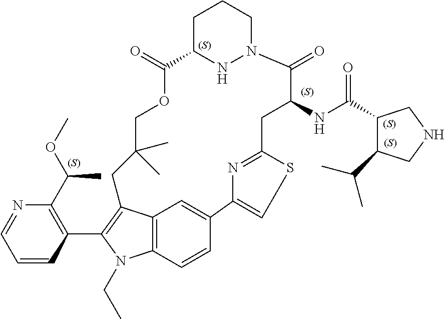 |
| DA 10 | 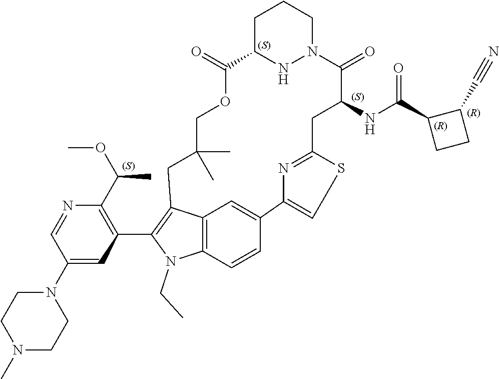 |
| DA 11 | 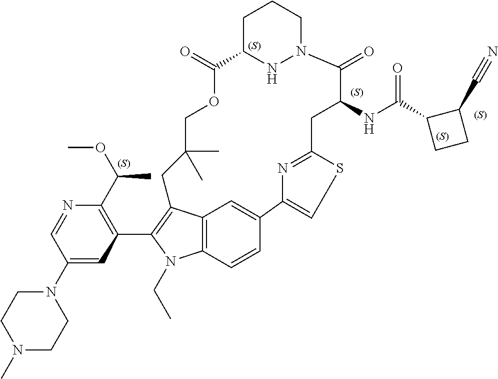 |
| DA 12 | 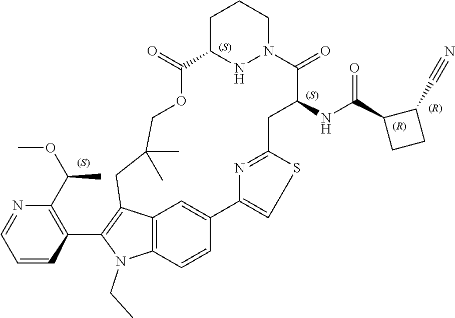 |
| DA 13 | 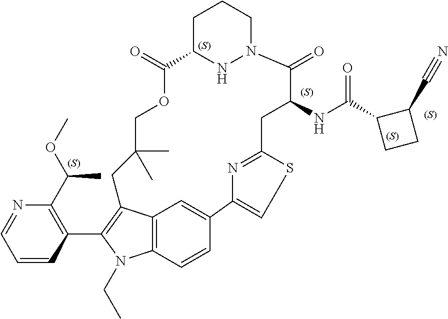 |
| DA 14 | 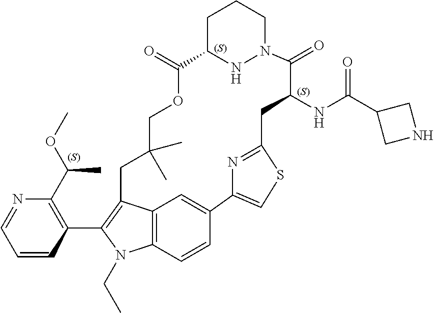 |
| DA 15 | 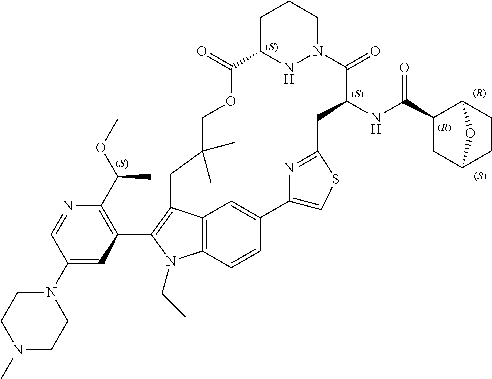 |
| DA 16 | 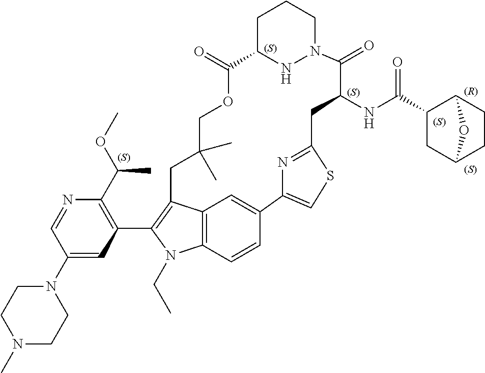 |
| DA 17 | 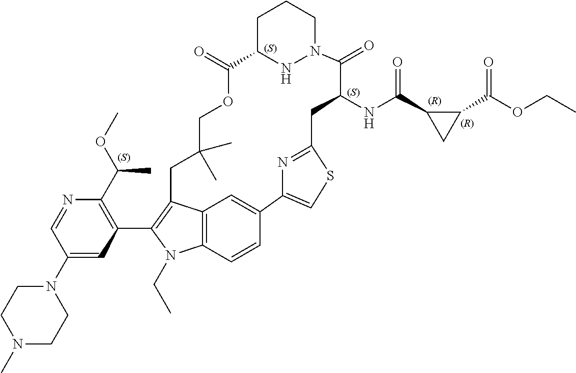 |
| DA 18 | 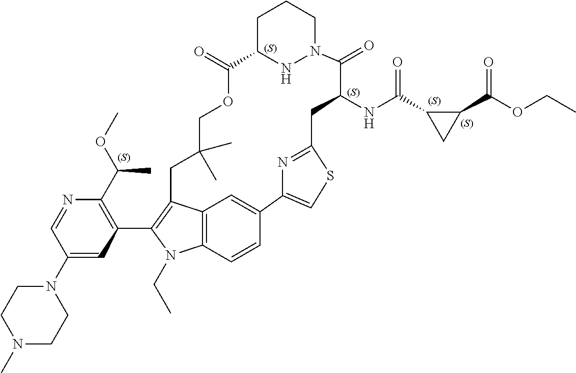 |
| DA 19 | 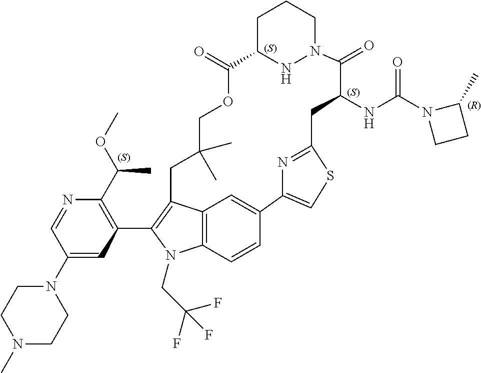 |
| DA 20 | 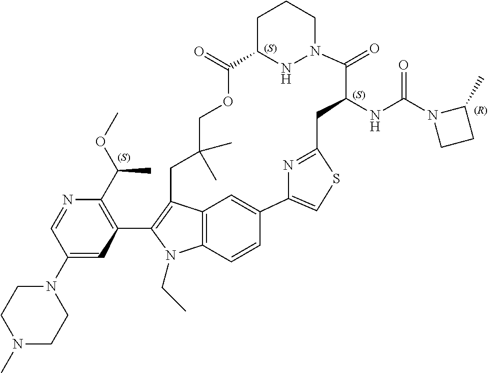 |
| DA 21 | 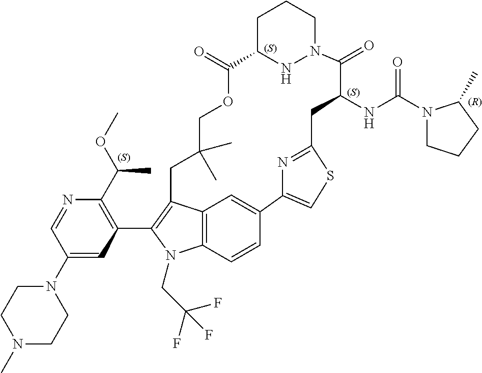 |
| DA 22 | 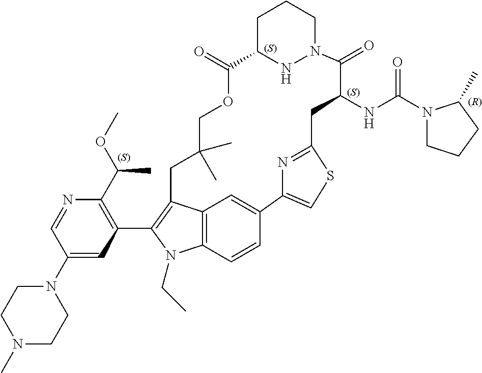 |
| DA 23 | 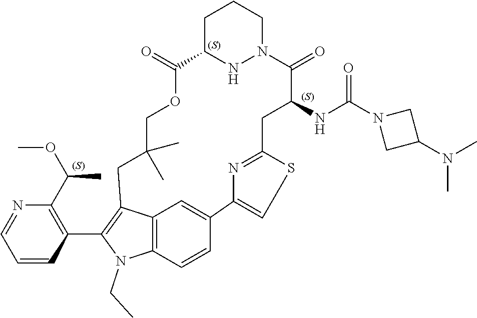 |
| DA 24 | 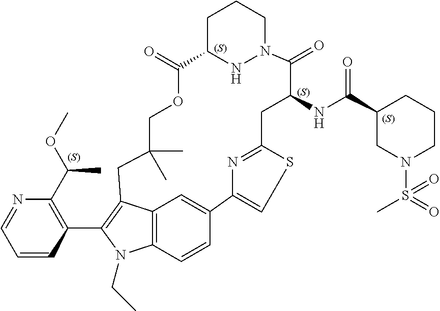 |
| DA 25 | 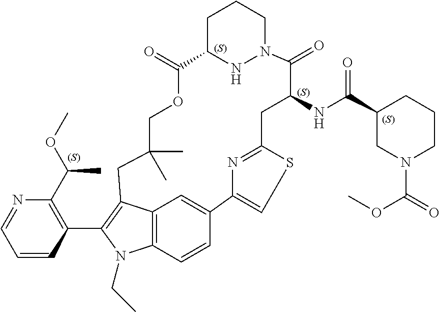 |
| DA 26 | 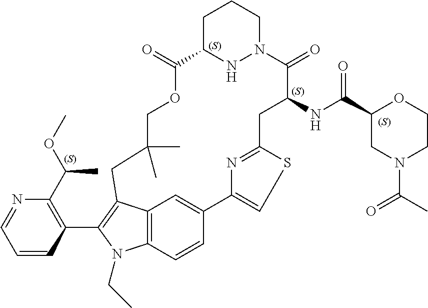 |
| DA 27 | 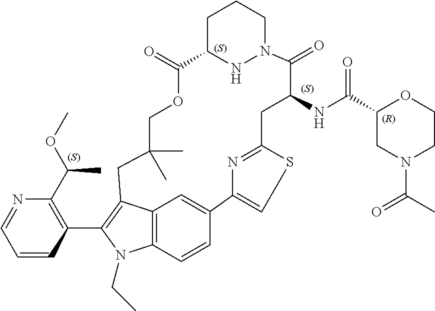 |
| DA 28 | 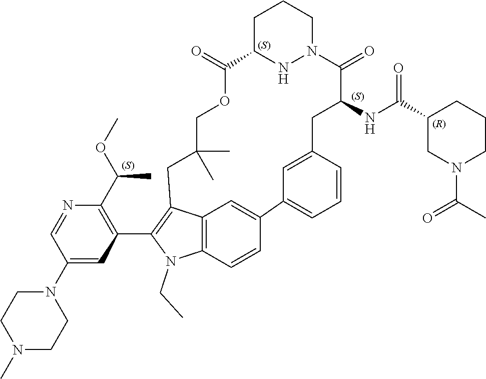 |
| DA 29 | 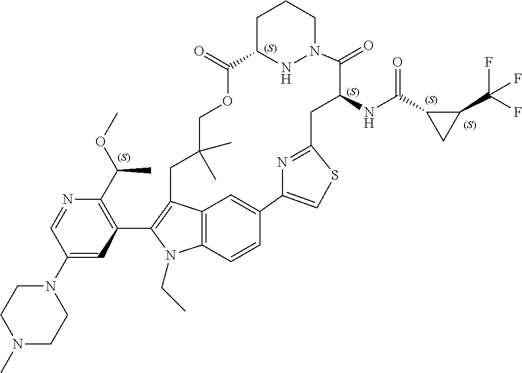 |
| DA 30 | 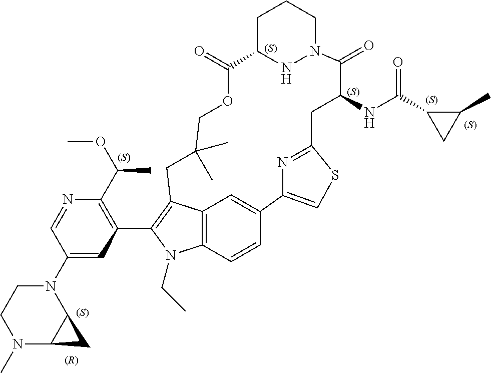 |
| DA 31 | 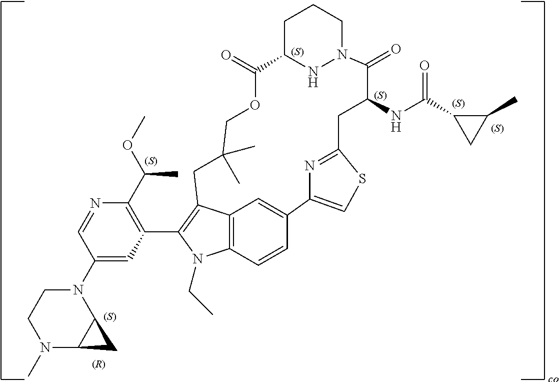 |
| DA 32 | 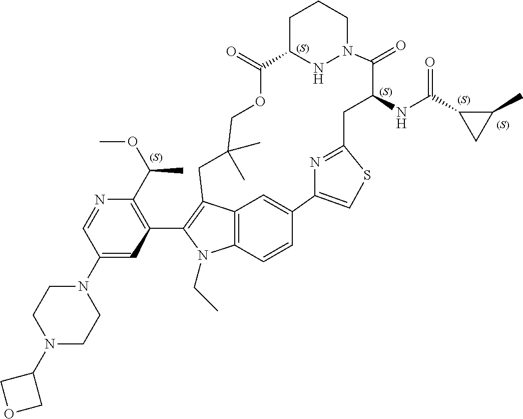 |
| DA 33 | 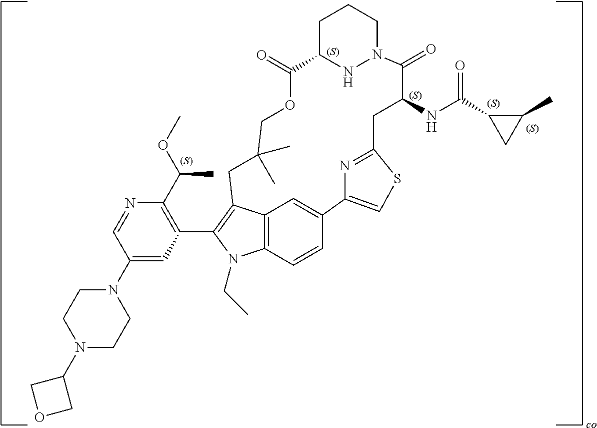 |
| DA 34 | 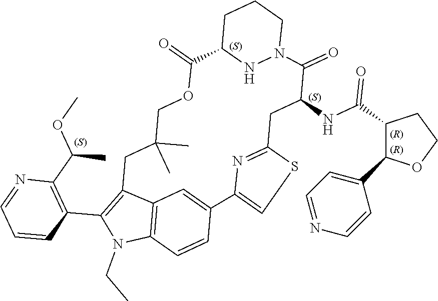 |
| DA 35 | 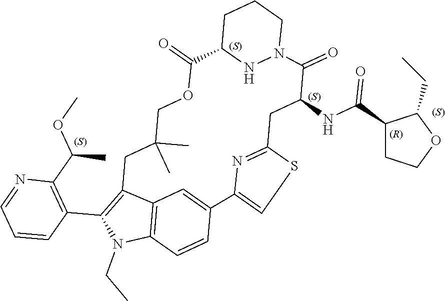 |
| DA 36 | 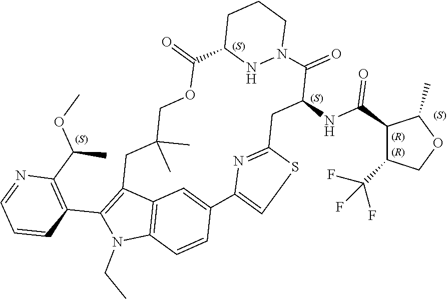 |
| DA 37 | 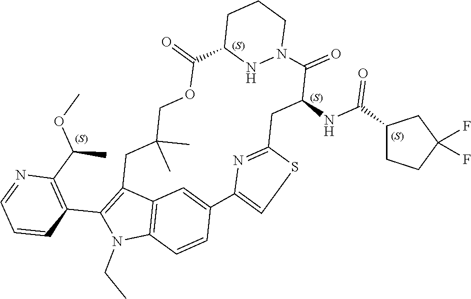 |
| DA 38 | 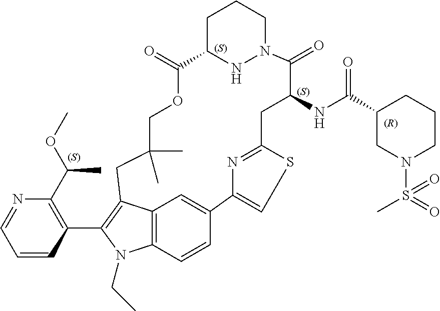 |
| DA 39 | 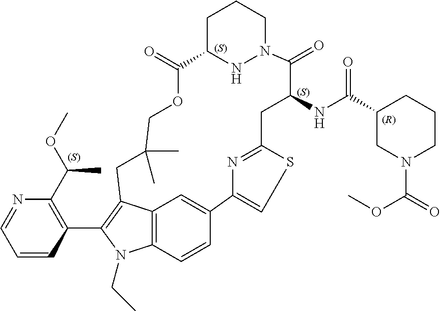 |
| DA 40 | 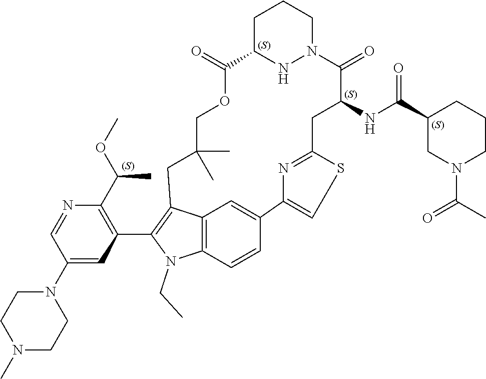 |
| DA 41 | 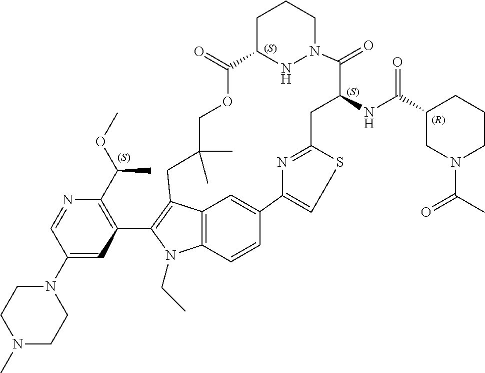 |
| DA 42 | 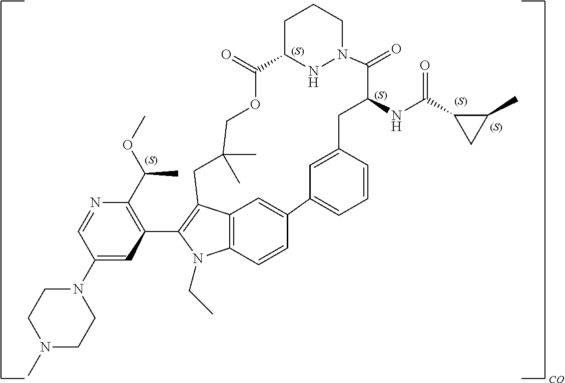 |
| DA 43 | 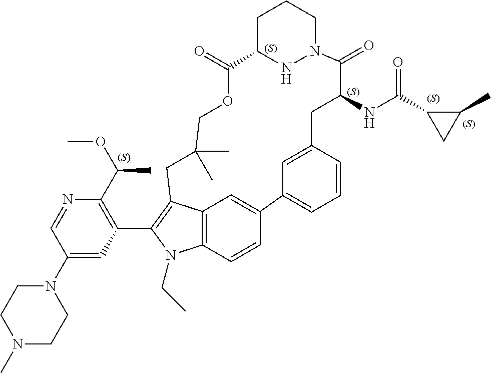 |
| DA 44 | 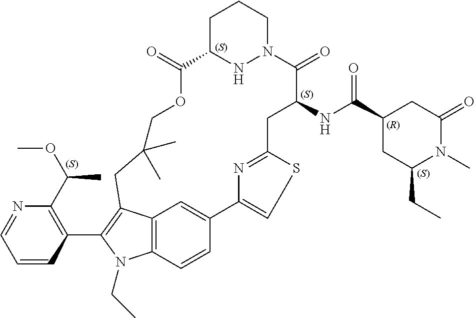 |
| DA 45 | 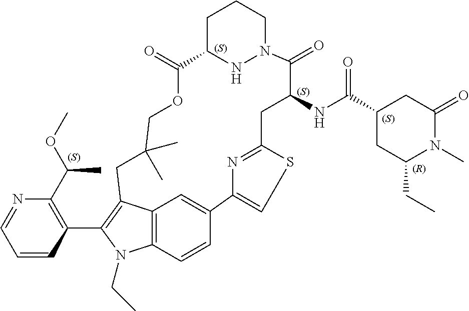 |
| DA 46 | 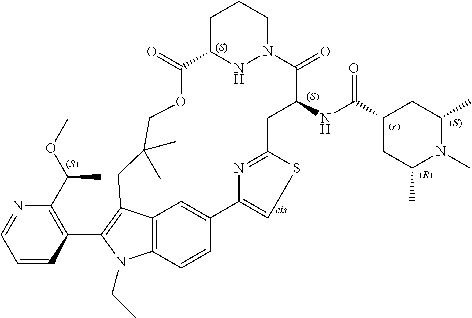 |
| DA 47 | 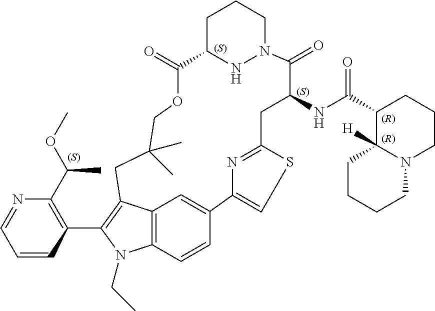 |
| DA 48 | 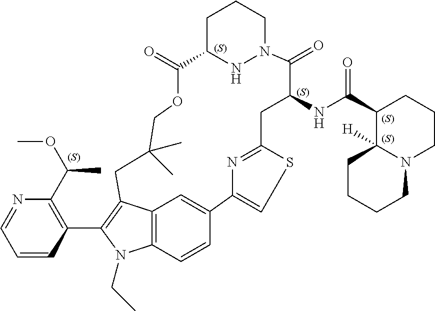 |
| DA 49 | 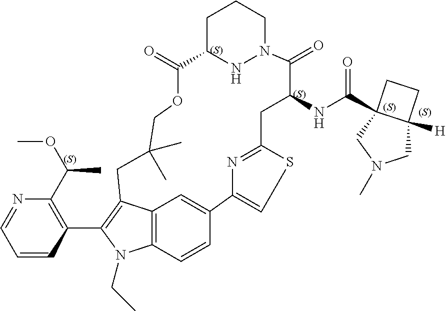 |
| DA 50 | 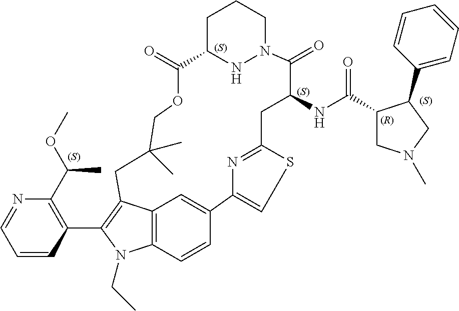 |
| DA 51 | 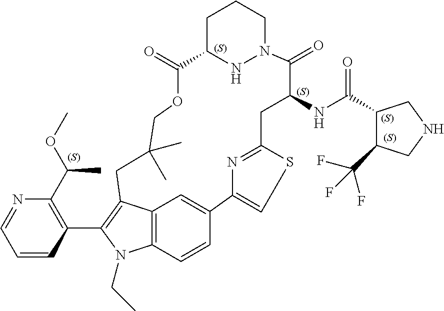 |
| DA 52 | 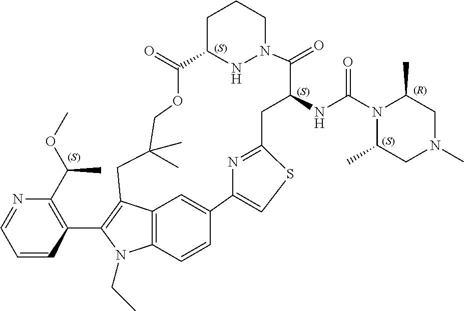 |
| DA 53 | 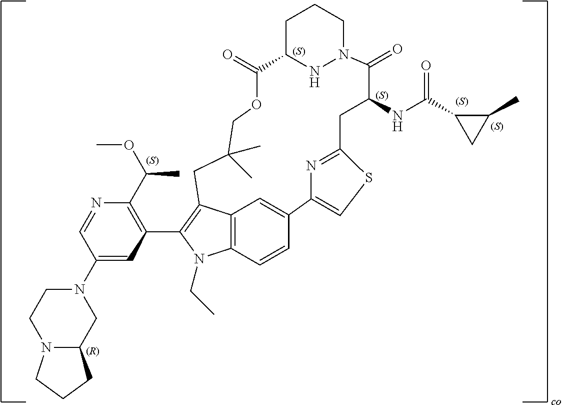 |
| DA 54 | 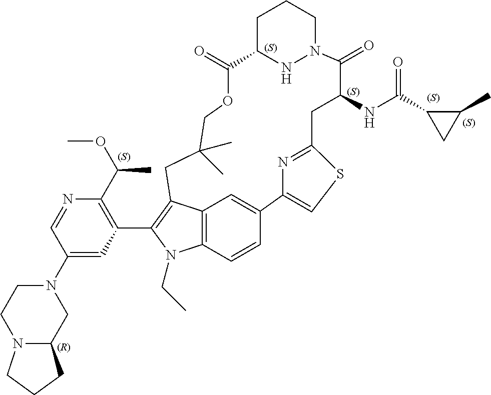 |
| DA 55 | 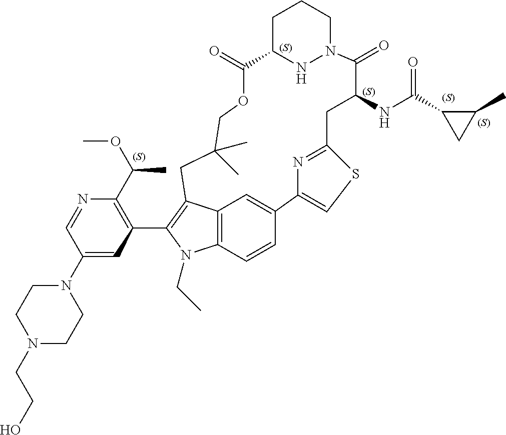 |
| DA 56 | 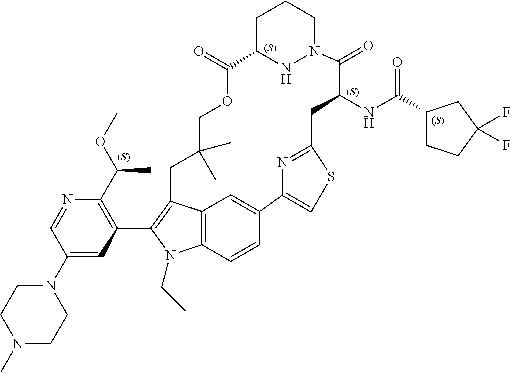 |
| DA 57 | 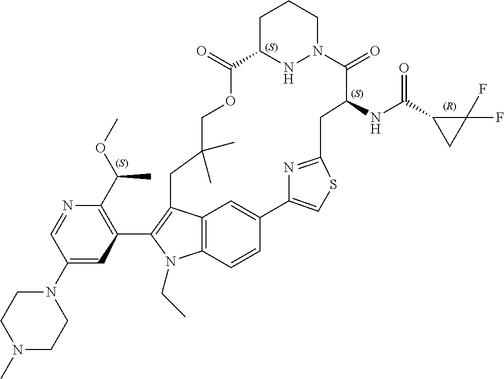 |
| DA 58 | 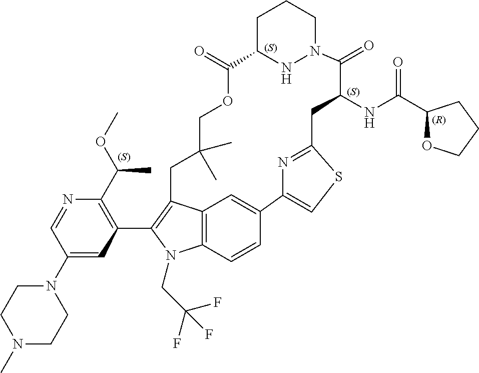 |
| DA 59 | 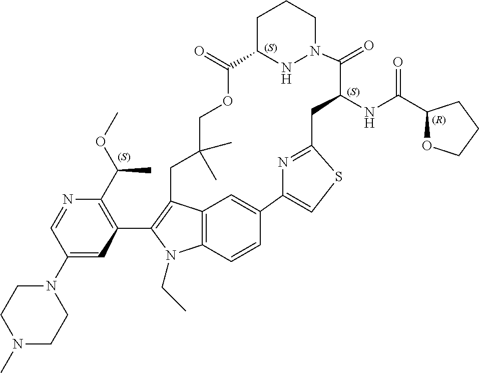 |
| DA 60 | 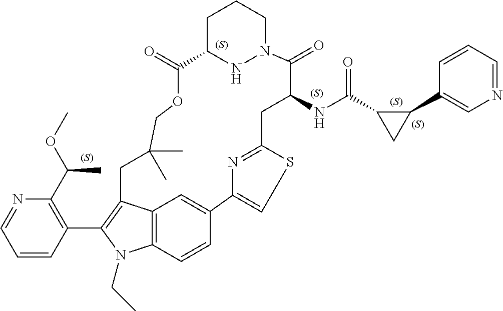 |
| DA 61 | 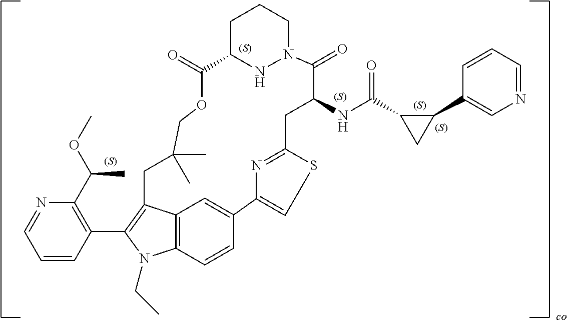 |
| DA 62 | 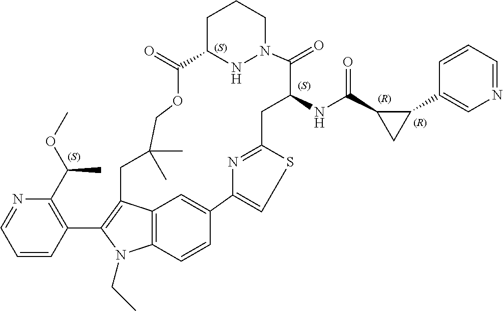 |
| DA 63 | 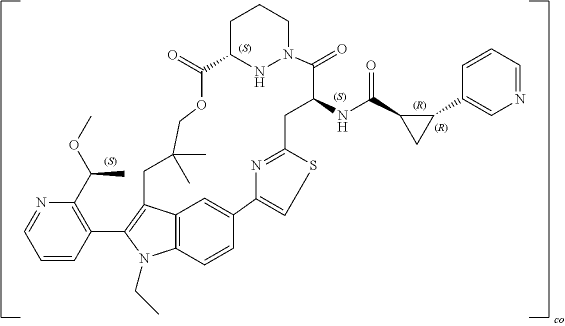 |
| DA 64 | 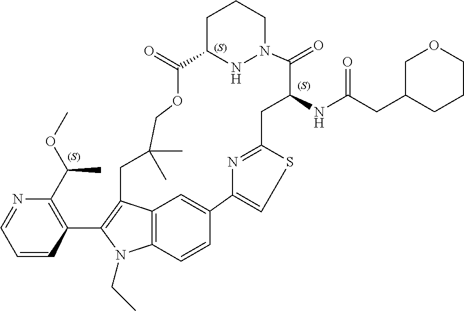 |
| DA 65 | 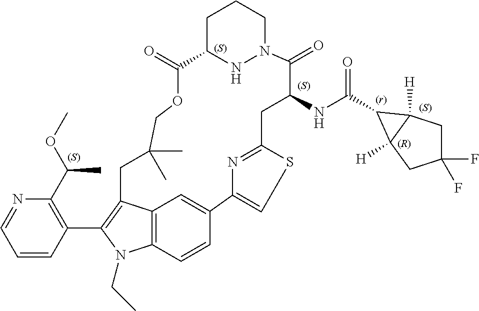 |
| DA 66 | 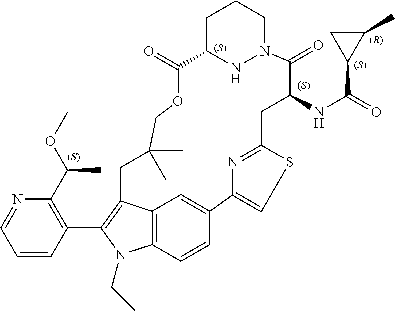 |
| DA 67 | 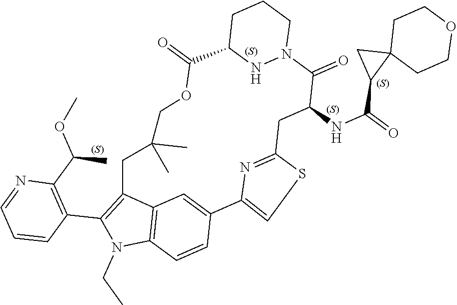 |
| DA 68 | 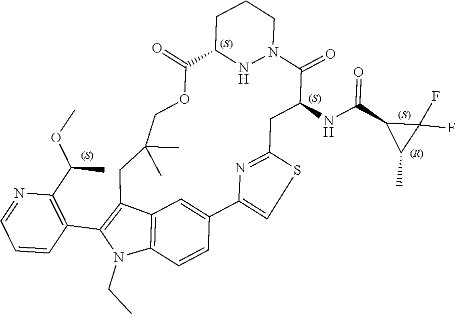 |
| DA 69 | 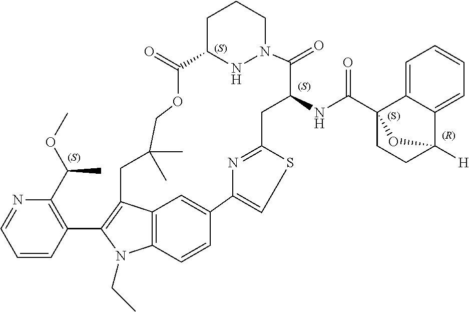 |
| DA 70 | 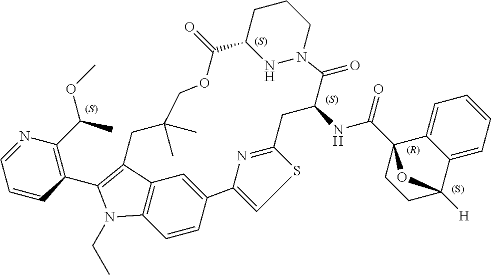 |
| DA 71 | 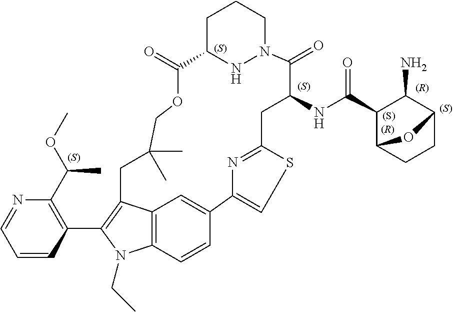 |
| DA 72 | 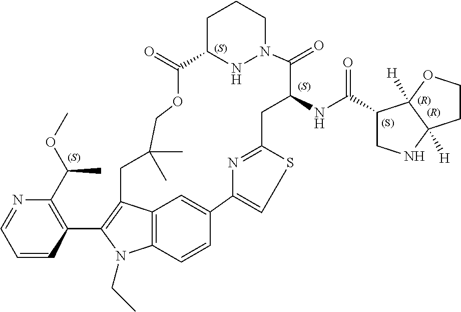 |
| DA 73 | 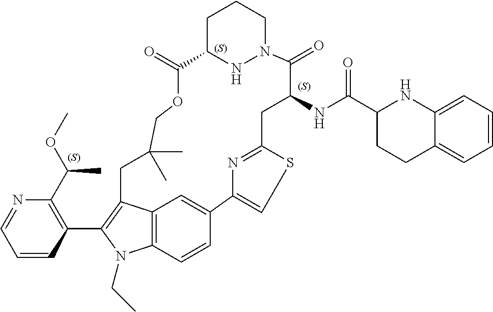 |
| DA 74 | 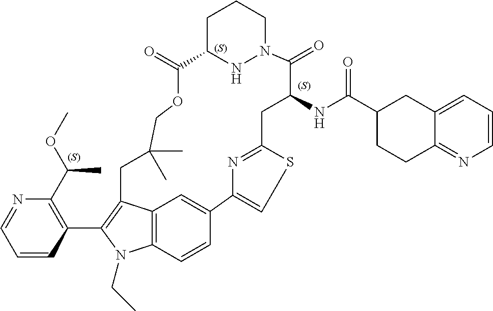 |
| DA 75 | 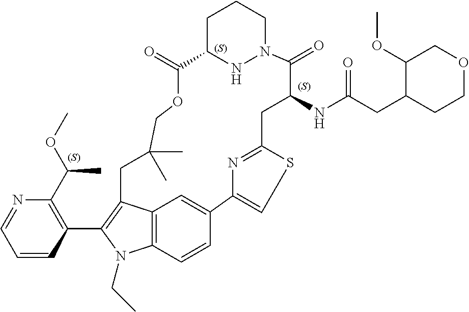 |
| DA 76 | 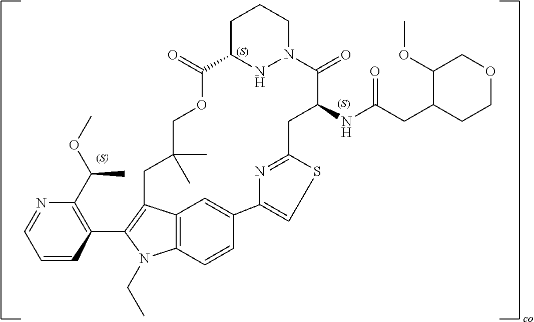 |
| DA 77 | 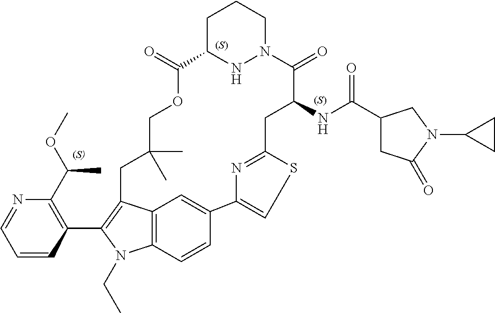 |
| DA 78 | 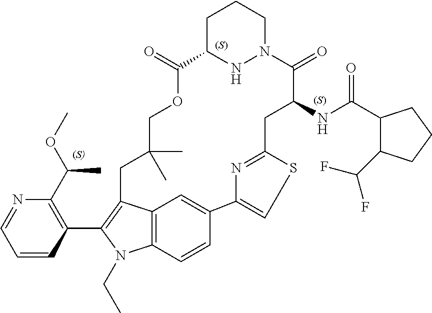 |
| DA 79 | 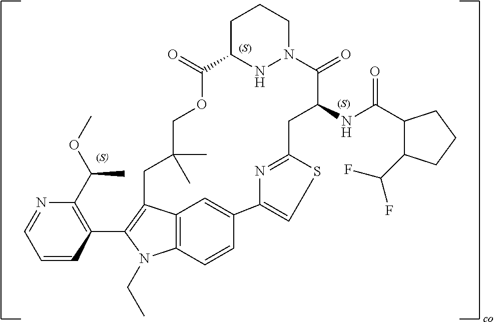 |
| DA 80 | 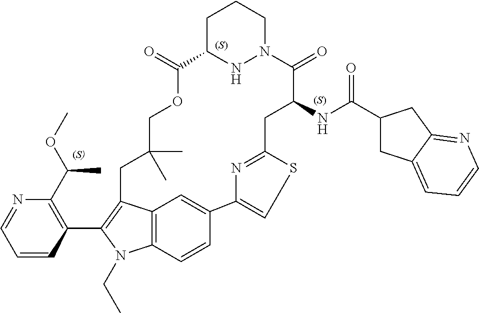 |
| DA 81 | 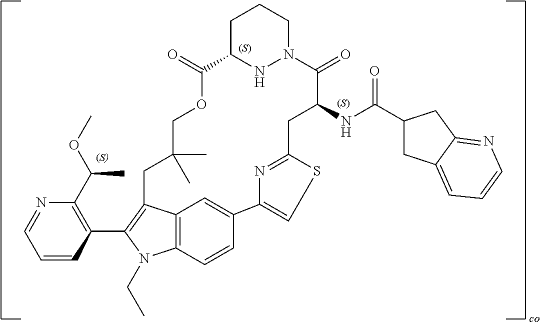 |
| DA 82 | 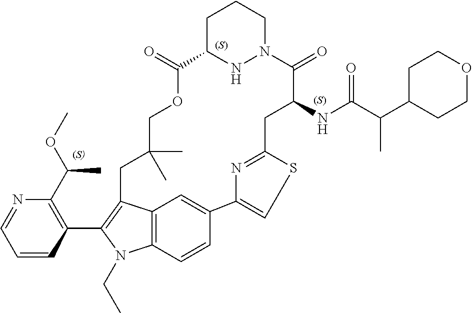 |
| DA 83 | 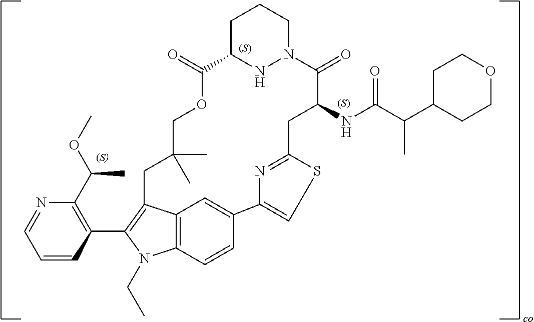 |
| DA 84 | 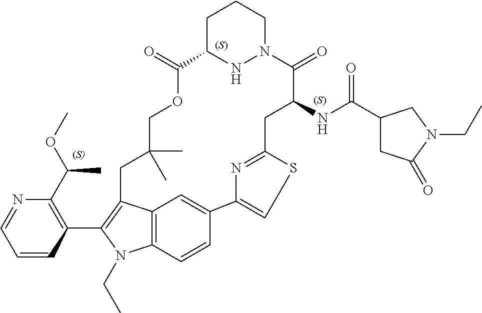 |
| DA 85 | 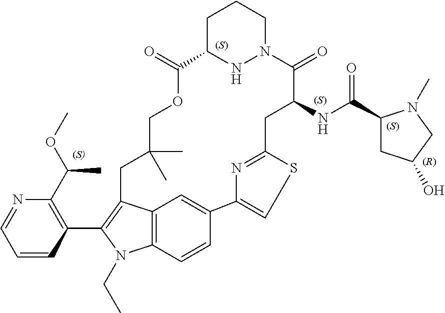 |
| DA 86 | 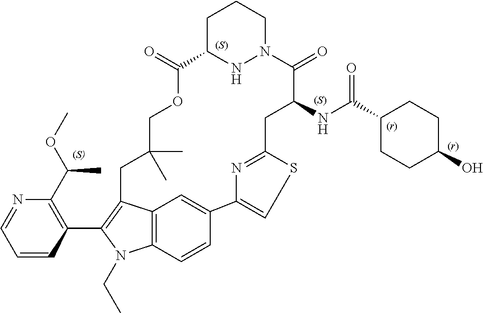 |
| DA 87 | 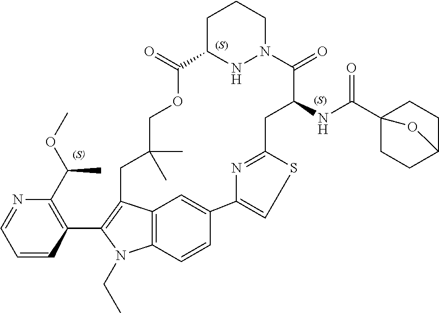 |
| DA 88 | 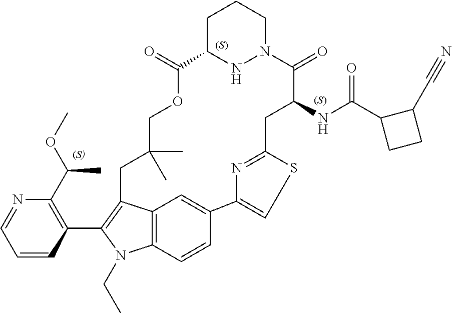 |
| DA 89 | 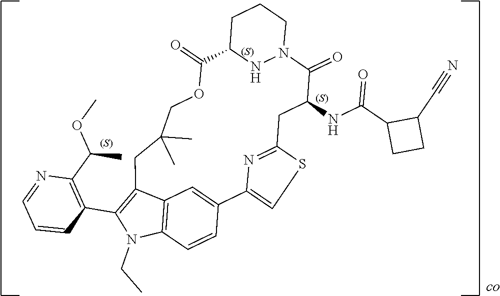 |
| DA 90 | 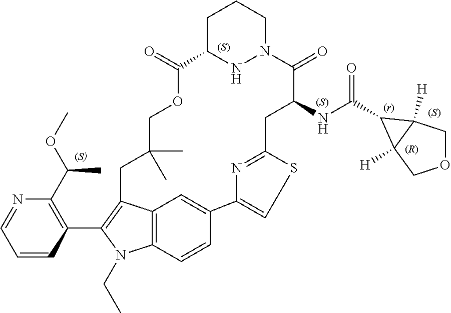 |
| DA 91 | 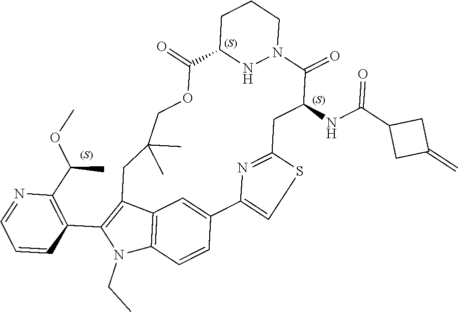 |
| DA 92 | 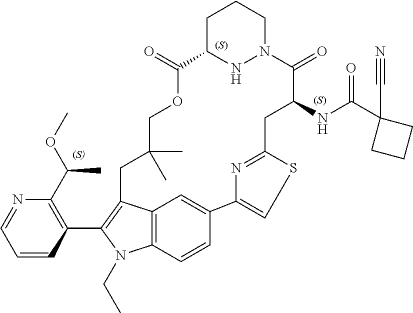 |
| DA 93 | 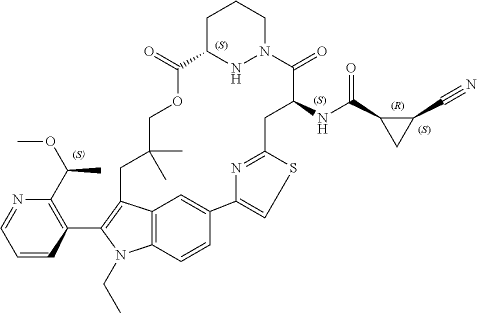 |
| DA 94 | 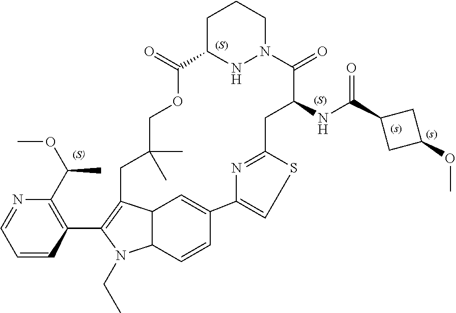 |
| DA 95 | 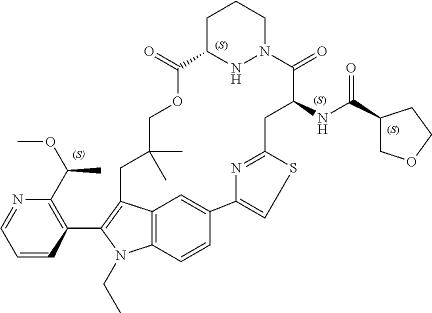 |
| DA 96 | 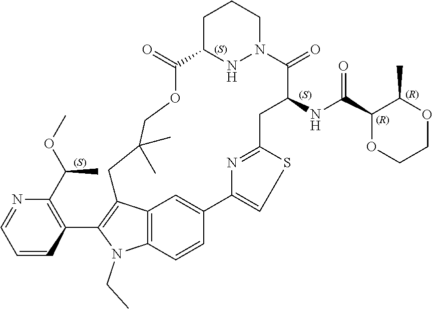 |
| DA 97 | 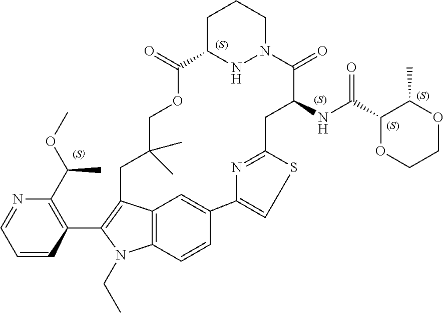 |
| DA 98 | 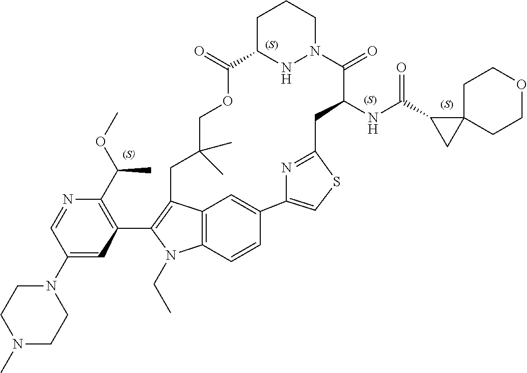 |
| DA 99 | 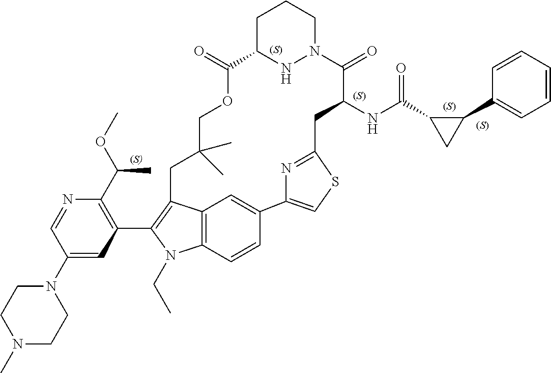 |
| DA 100 | 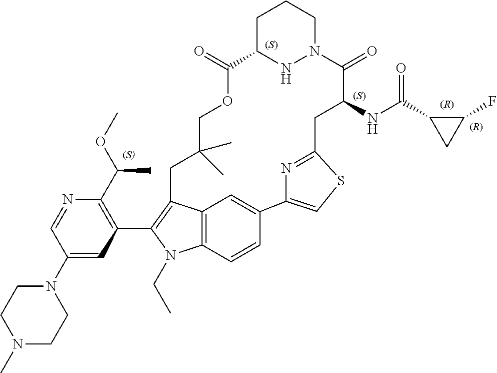 |
| DA 101 | 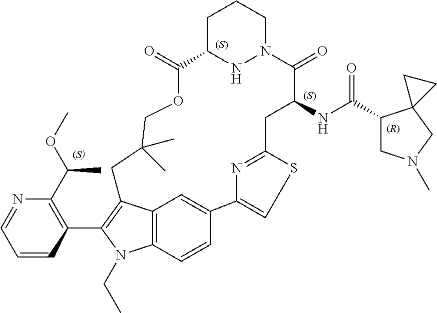 |
| DA 102 | 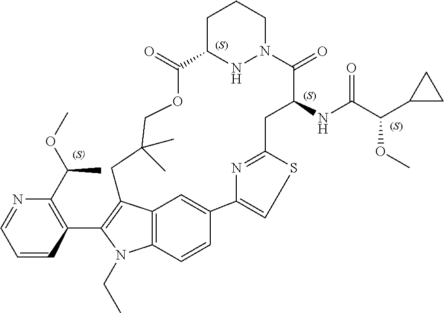 |
| DA 103 | 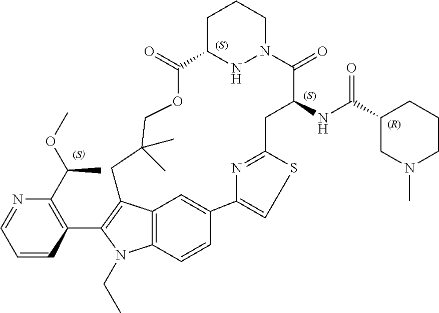 |
| DA 104 | 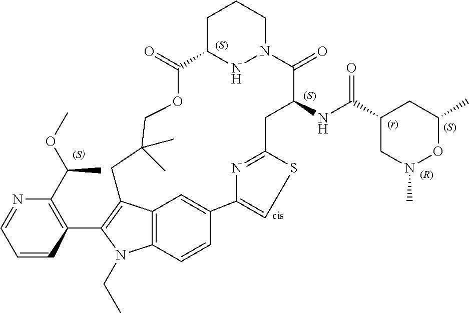 |
| DA 105 | 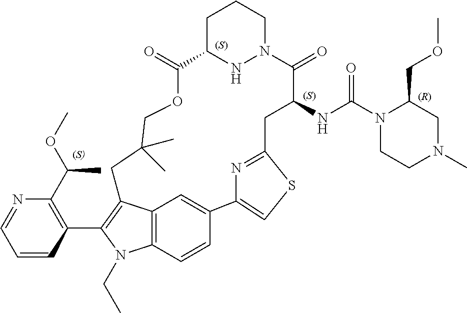 |
| DA 106 | 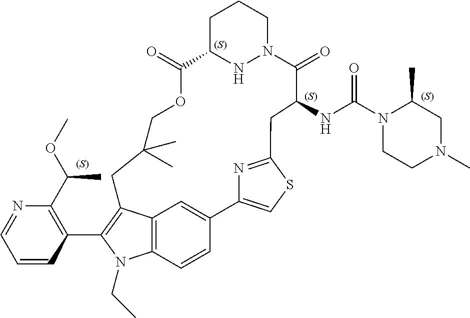 |
| DA 107 | 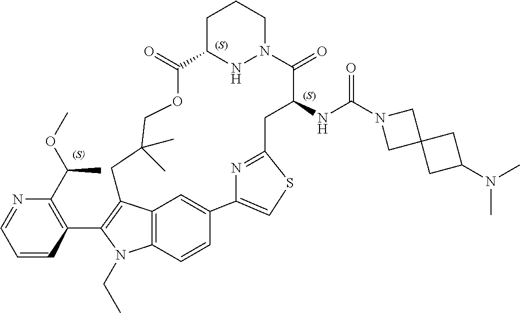 |
| DA 108 | 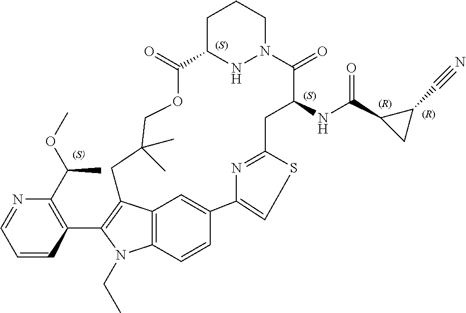 |
| DA 109 | 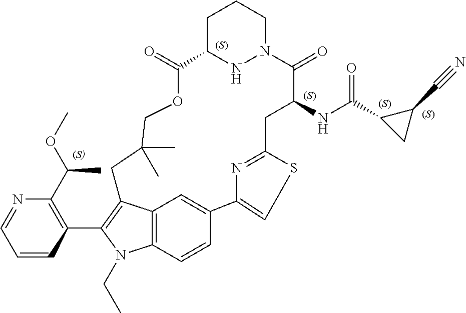 |
| DA 110 | 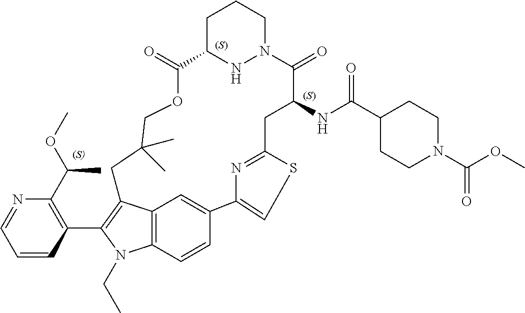 |
| DA 111 | 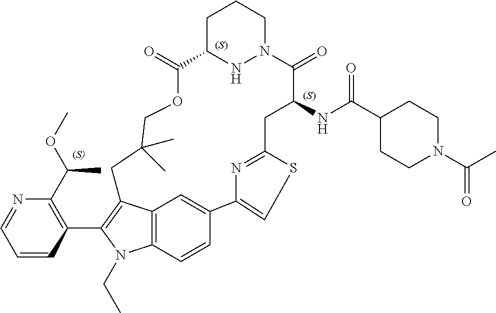 |
| DA 112 | 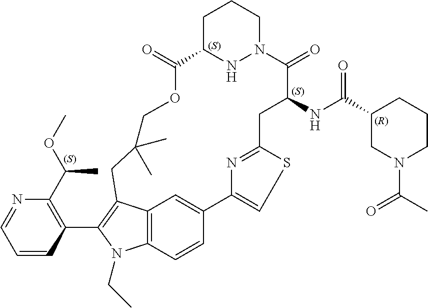 |
| DA 113 | 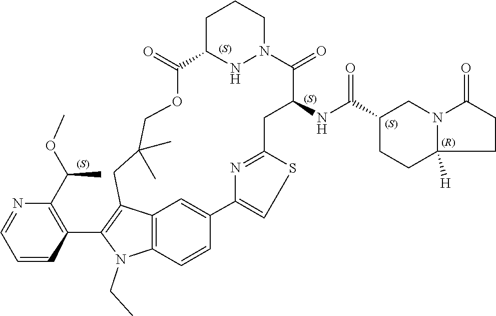 |
| DA 114 | 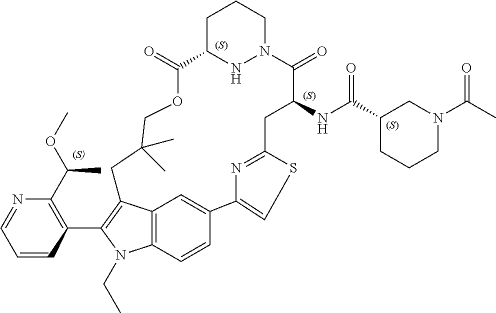 |
| DA 115 | 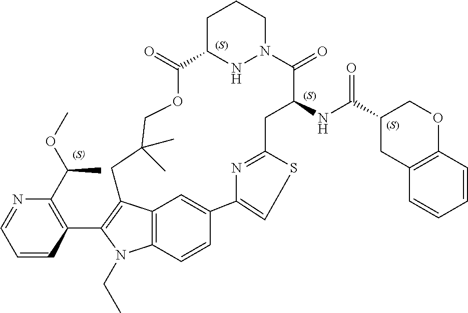 |
| DA 116 | 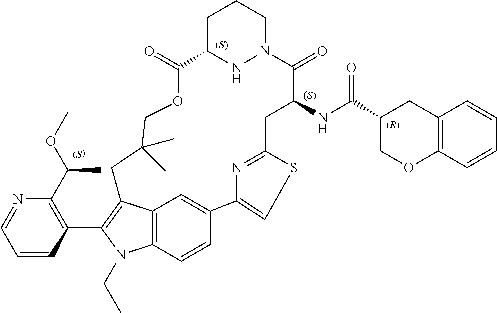 |
| DA 117 | 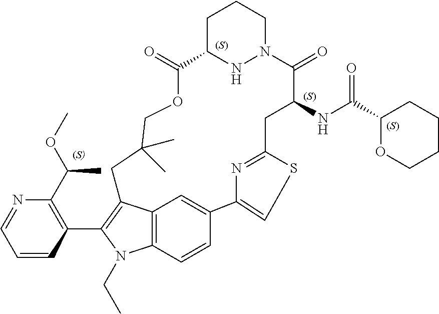 |
| DA 118 | 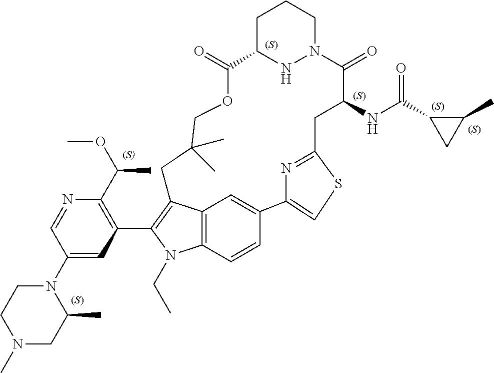 |
| DA 119 | 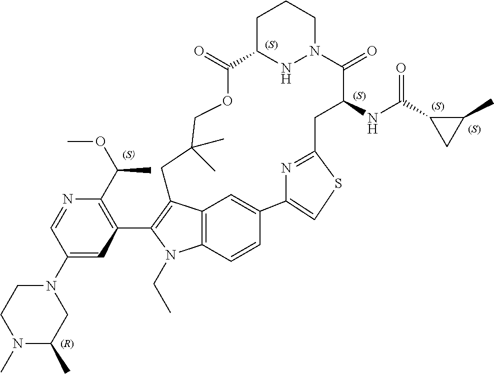 |
| DA 120 | 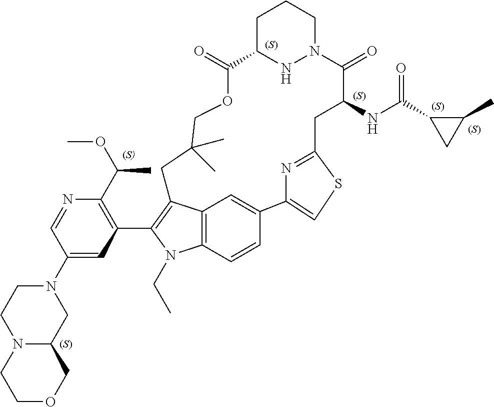 |
| DA 121 | 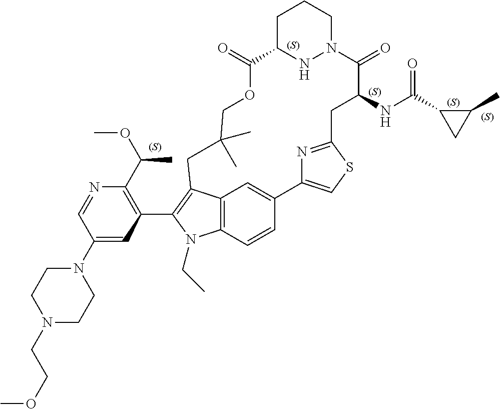 |
| DA 122 | 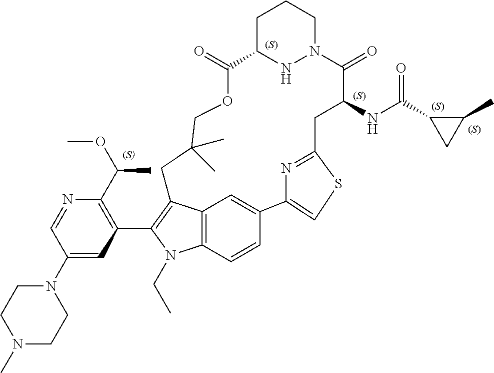 |
| DA 123 | 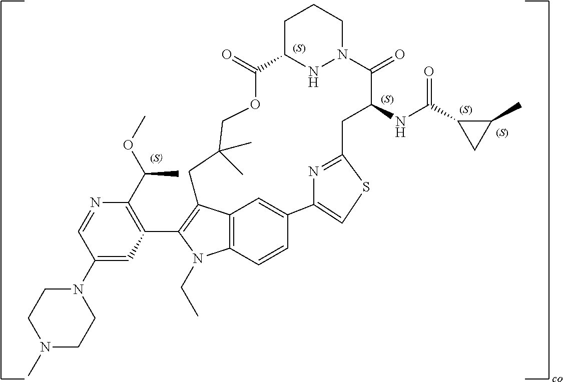 |
| DA 124 | 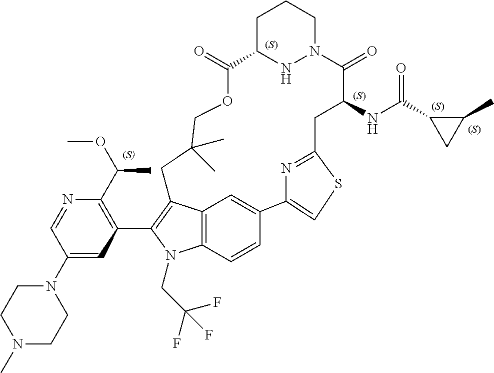 |
| DA 125 | 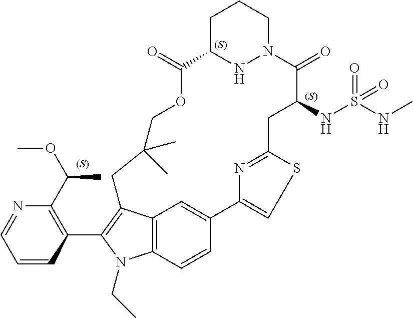 |
| DA 126 | 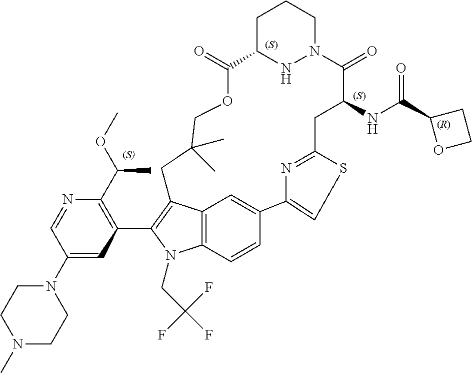 |
| DA 127 | 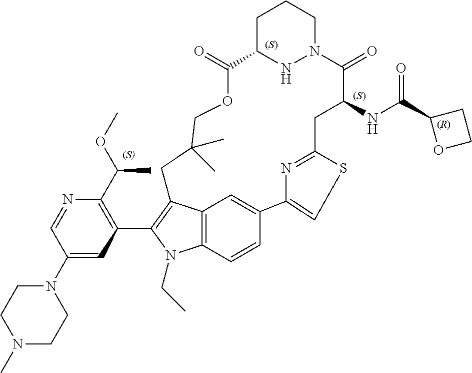 |
| DA 128 | 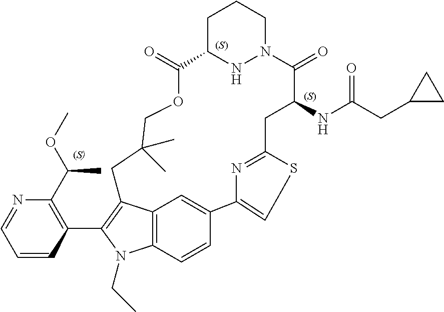 |
| DA 129 | 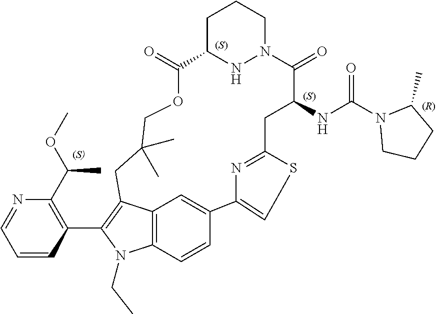 |
| DA 130 | 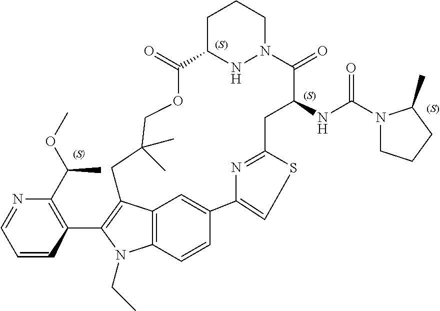 |
| DA 131 | 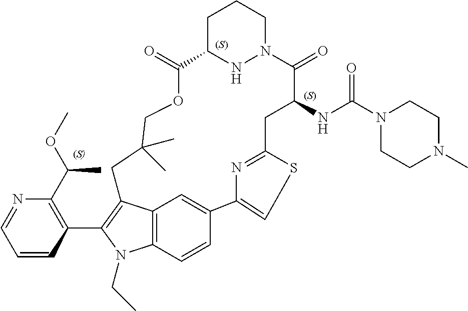 |
| DA 132 | 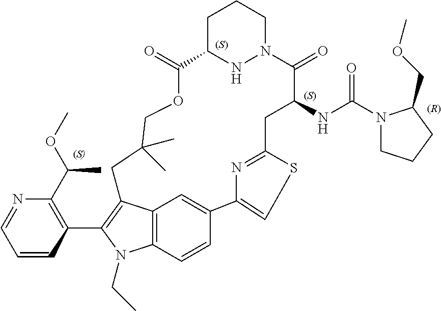 |
| DA 133 | 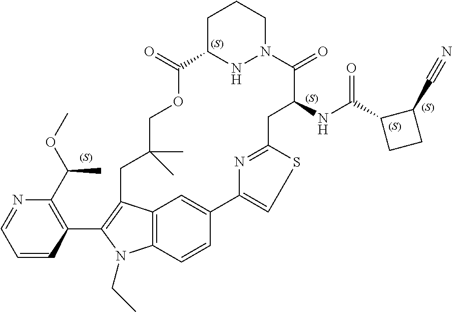 |
| DA 133 | 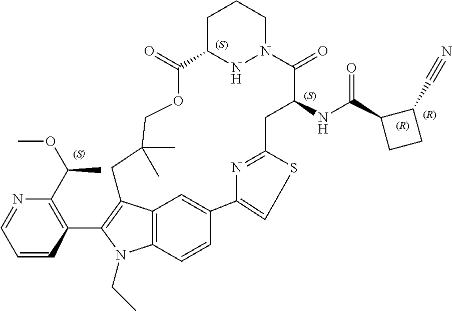 |
| DA 134 | 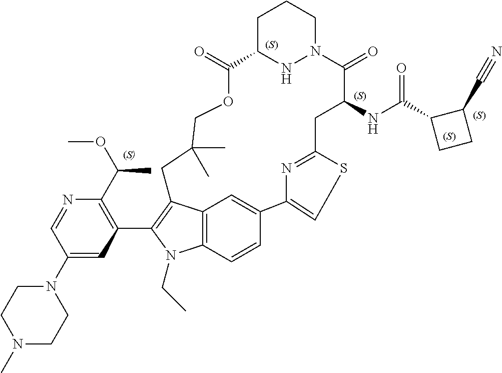 |
| DA 135 | 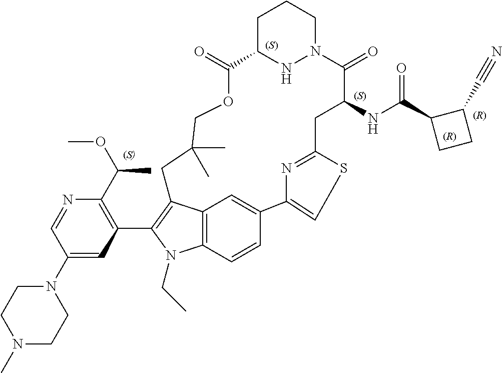 |
| DA 136 | 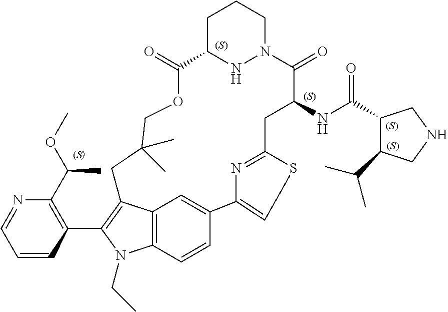 |
| DA 137 | 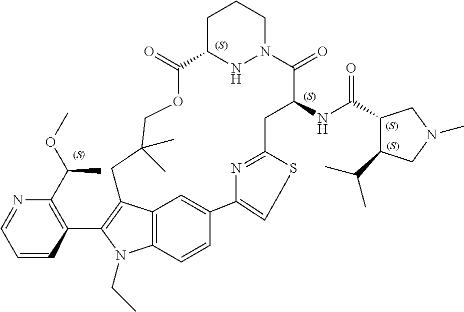 |
| DA 138 | 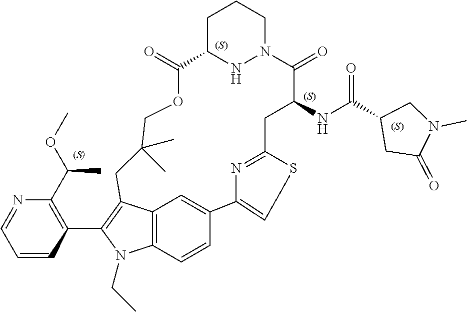 |
| DA 139 | 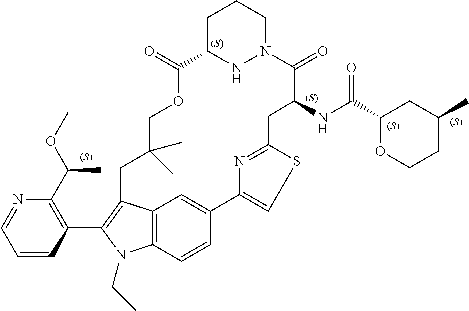 |
| DA 140 | 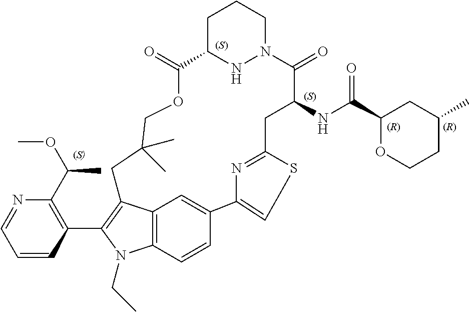 |
| DA 141 | 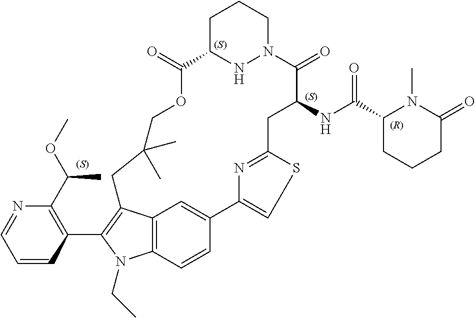 |
| DA 142 | 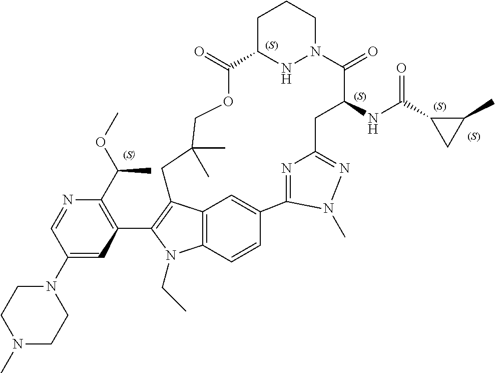 |
| DA 143 | 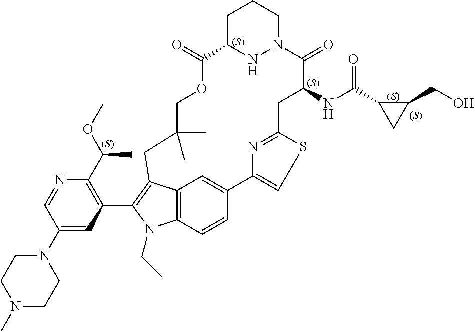 |
| DA 144 | 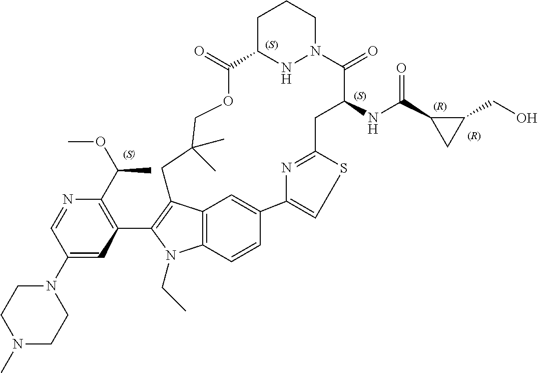 |
| DA 145 | 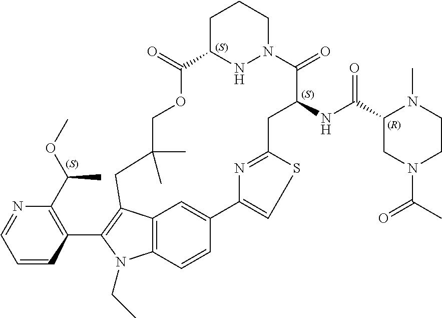 |
| DA 146 | 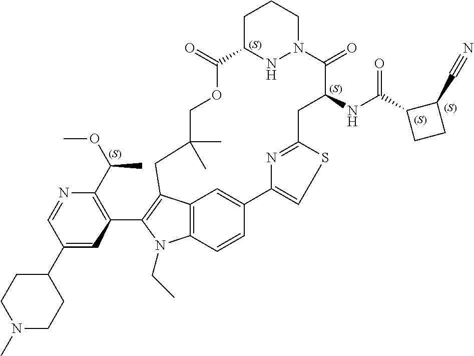 |
| DA 147 | 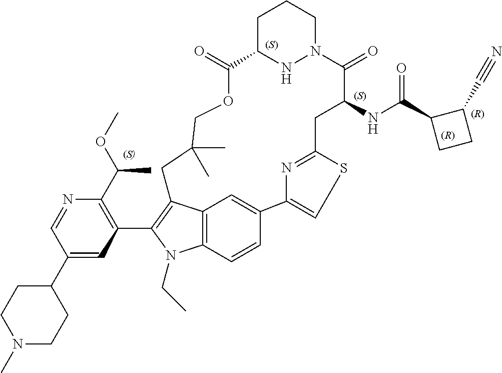 |
| DA 148 | 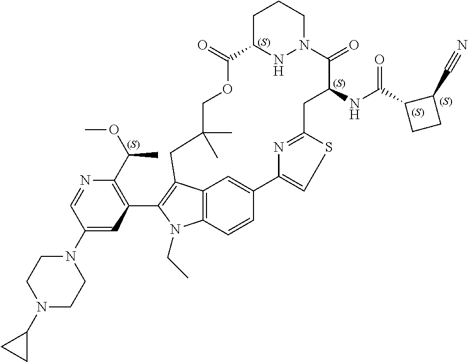 |
| DA 149 | 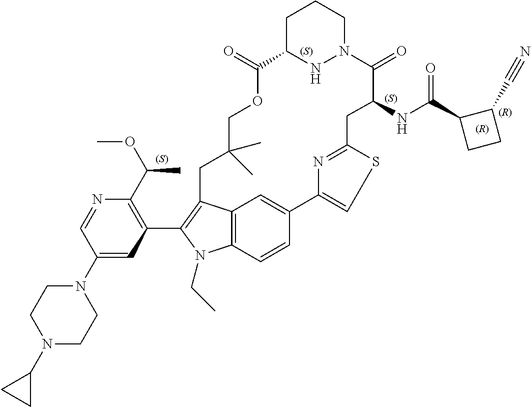 |
| DA 150 | 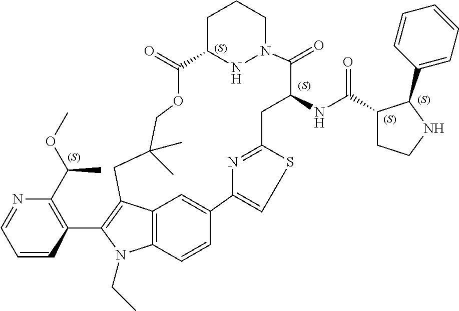 |
| DA 151 | 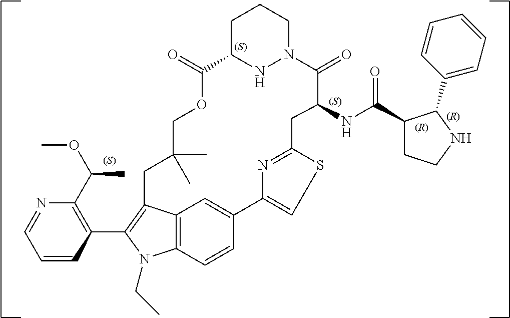 |
| DA 152 | 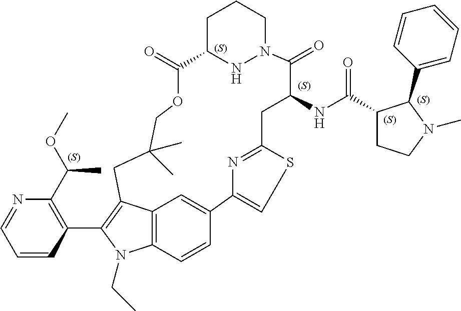 |
| DA 153 | 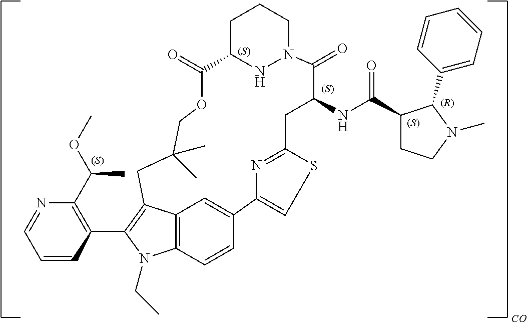 |
| DA 154 | 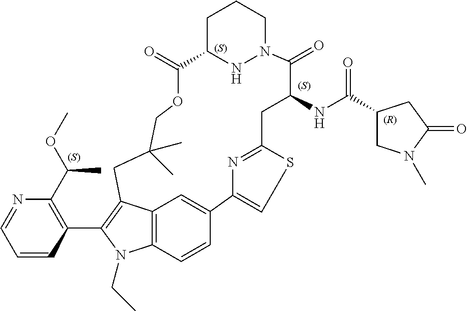 |
| DA 155 | 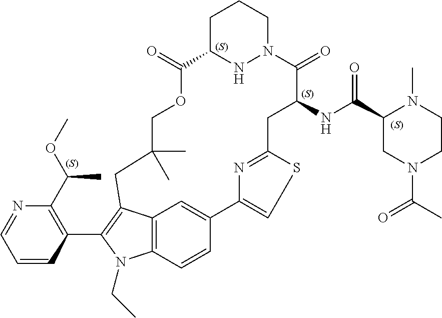 |
| DA 156 | 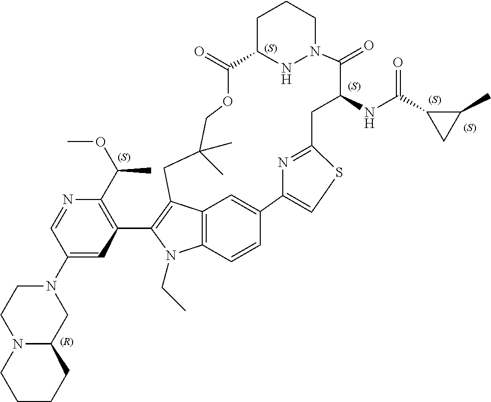 |
| DA 157 | 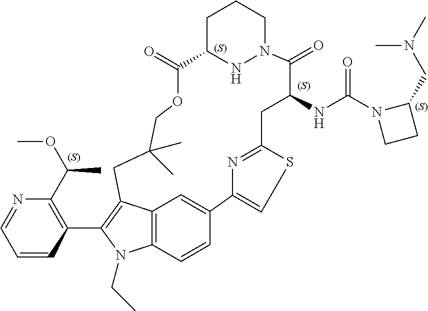 |
| DA 158 | 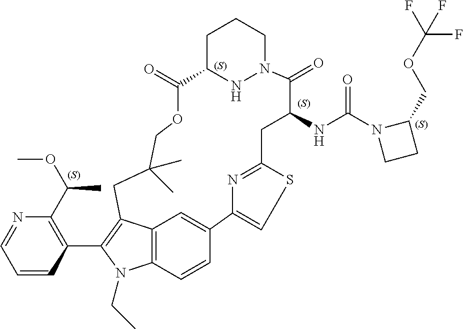 |
| DA 159 | 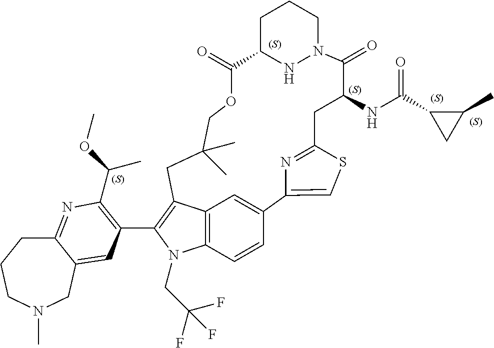 |
| DA 160 | 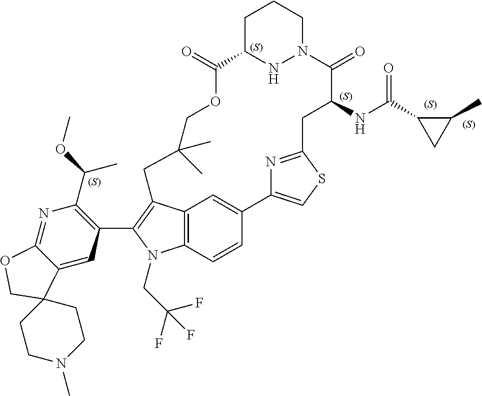 |
| DA 161 | 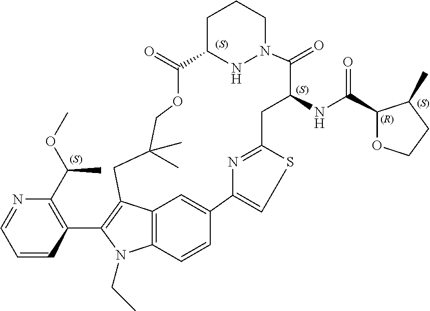 |
| DA 162 | 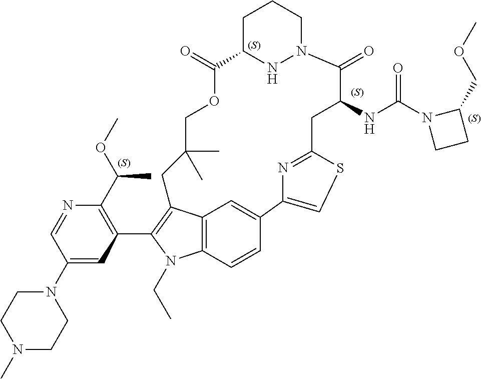 |
| DA 163 | 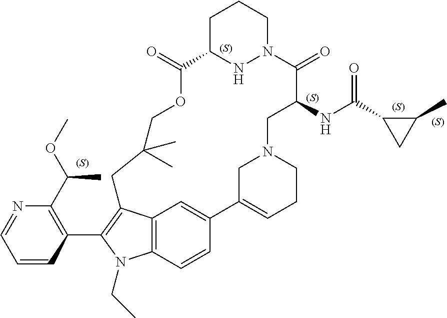 |
| DA 164 | 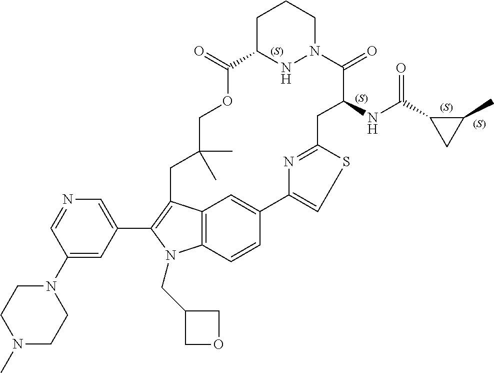 |
| DA 165 | 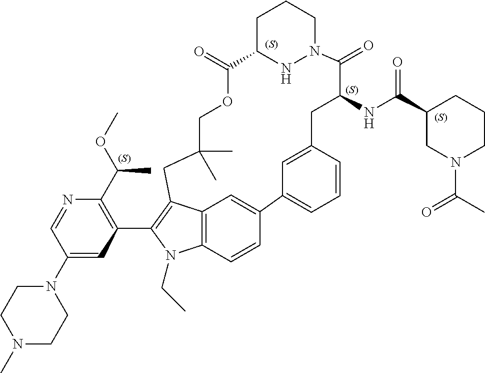 |
| DA 166 | 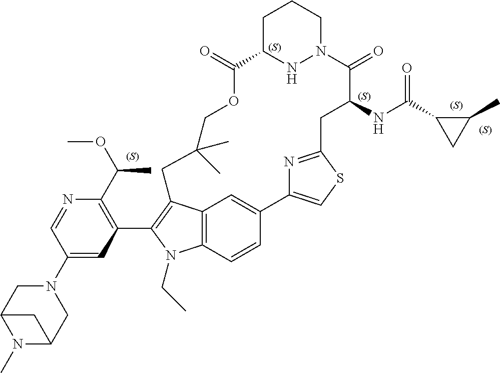 |
| DA 167 | 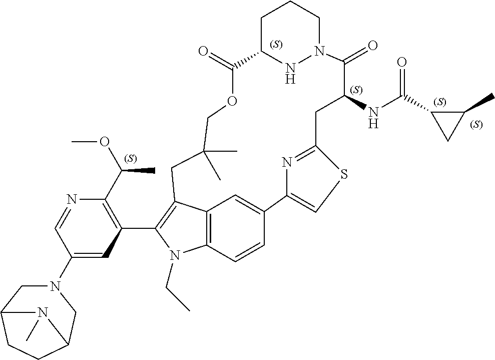 |
| DA 168 | 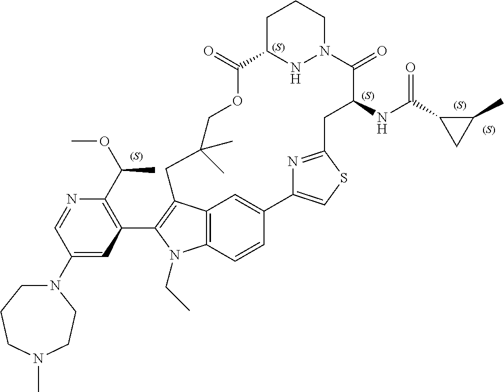 |
| DA 169 | 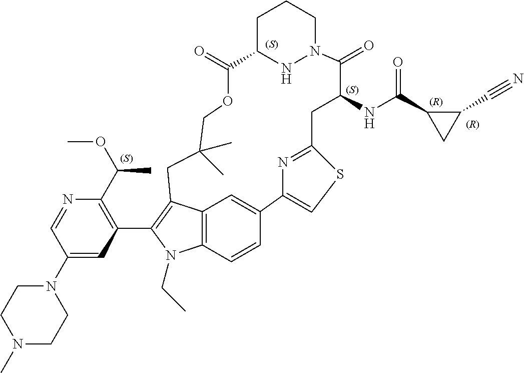 |
| DA 170 | 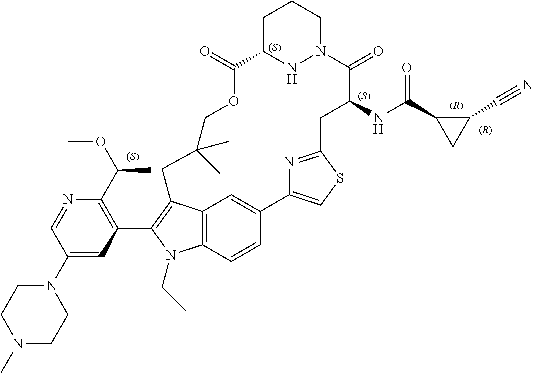 |
| DA 171 | 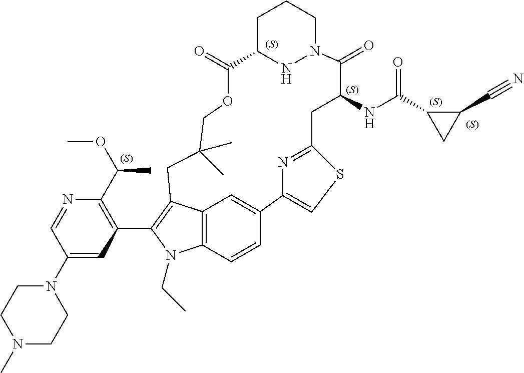 |
| DA 172 | 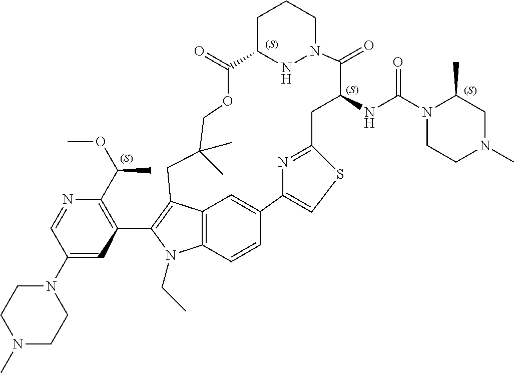 |
| DA 173 | 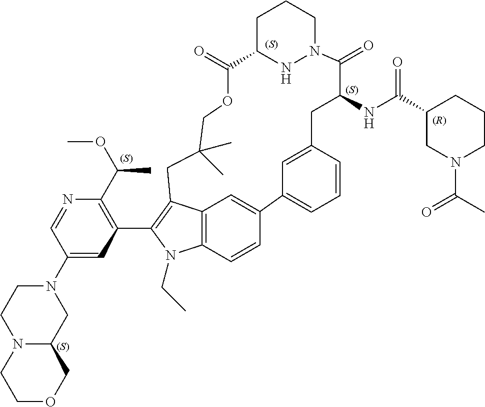 |
| DA 174 | 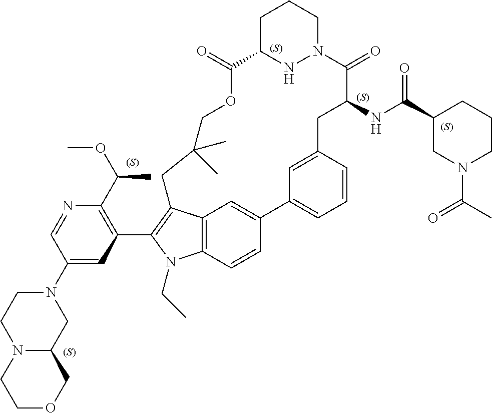 |
| DA 175 | 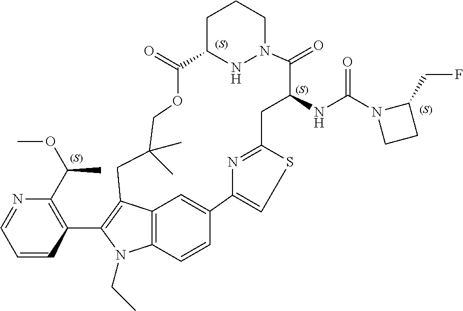 |
| DA 176 | 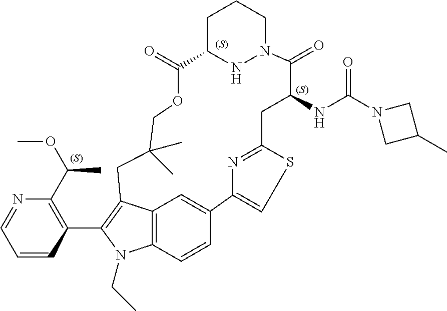 |
| DA 177 | 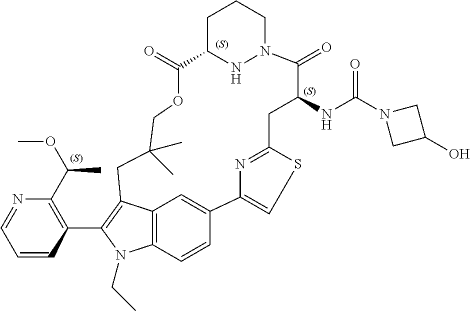 |
| DA 178 | 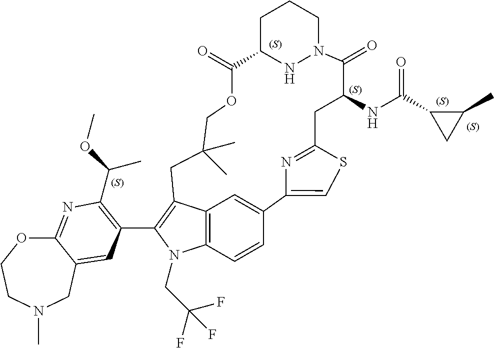 |
| DA 179 | 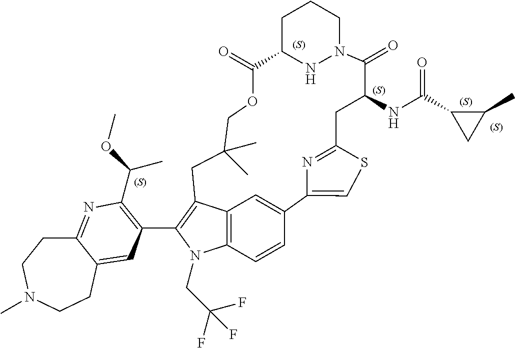 |
| DA 180 | 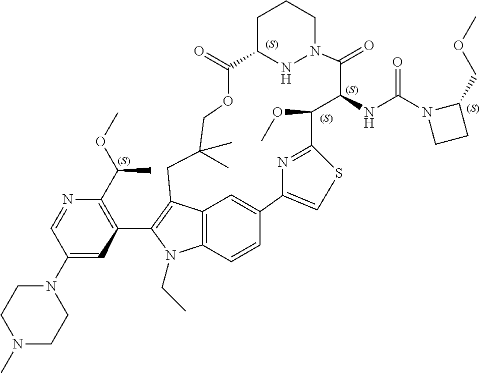 |
| DA 181 | 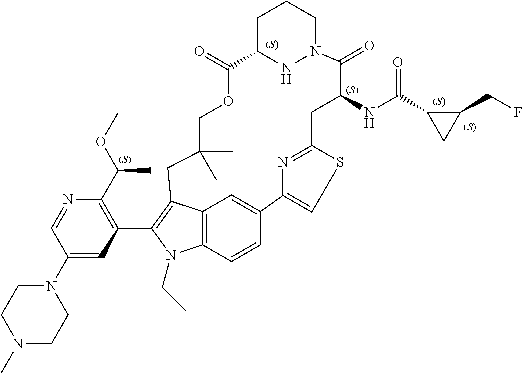 |
| DA 182 | 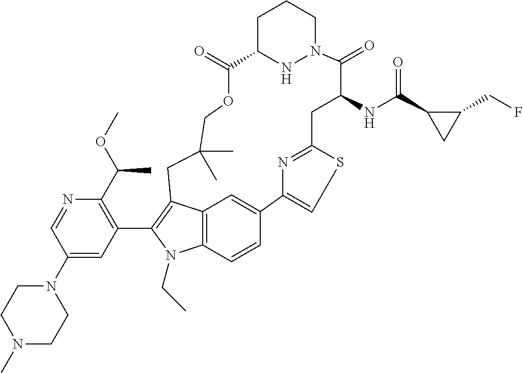 [SMILES: CCn1c(-c2cc(N3CCN(C)CC3)cnc2[C@H](C)OC)c2c3cc(ccc31)-c1csc(n1)C[C@H](NC(=O)[C@@H]1C[C@H]1CF)C(=O)N1CCC[C@H](N1)C(=O)OCC(C)(C)C2] |
| DA 183 | 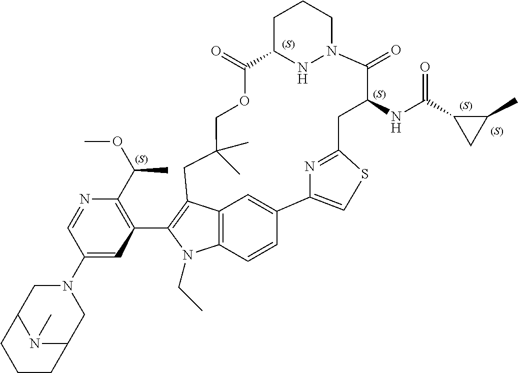 |
| DA 184 | 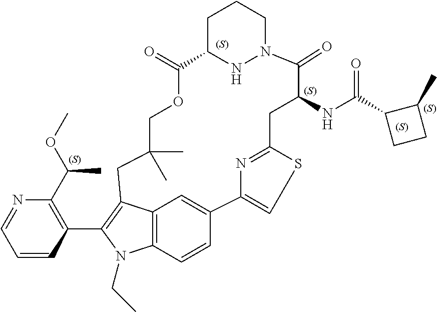 |
| DA 185 | 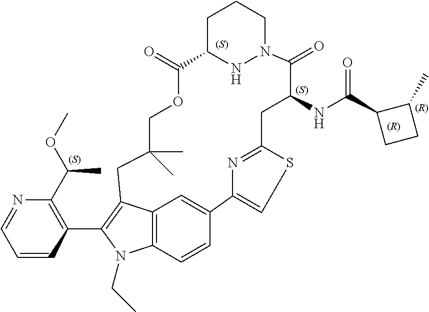 |
| DA 186 | 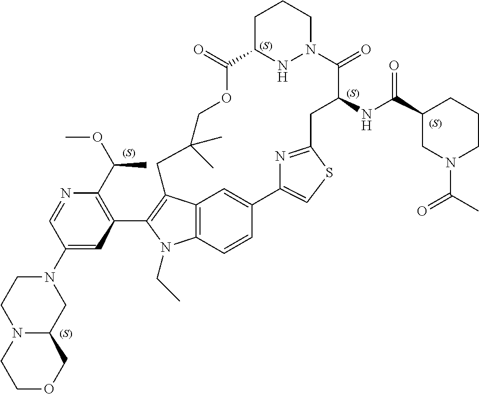 |
| DA 187 | 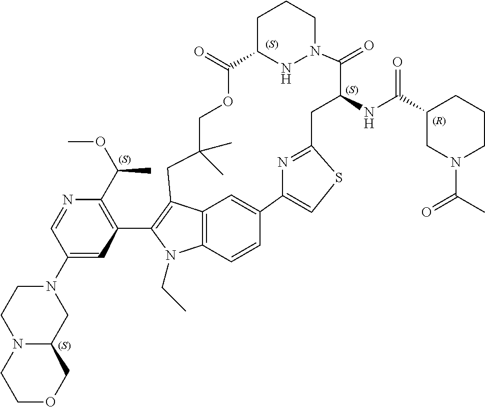 |
| DA 188 | 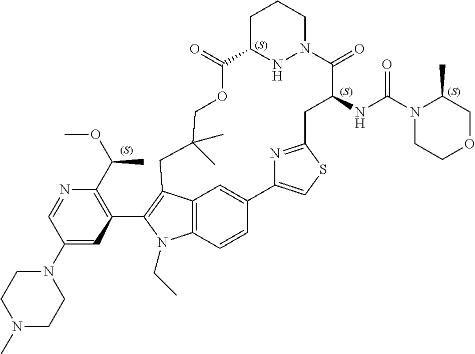 |
| DA 189 | 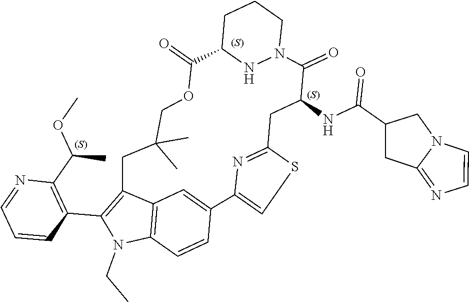 |
| DA 190 | 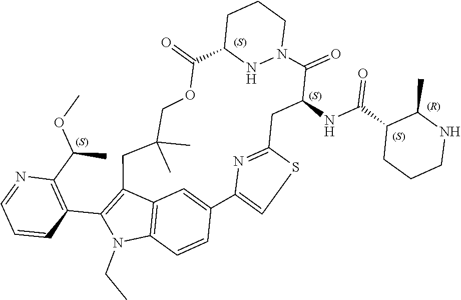 |
| DA 191 | 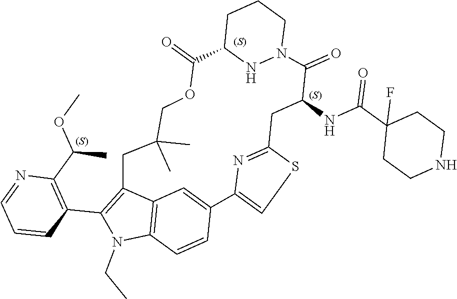 |
| DA 192 | 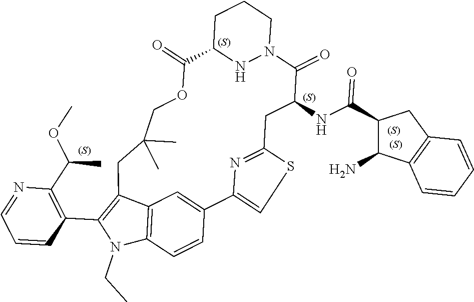 |
| DA 193 | 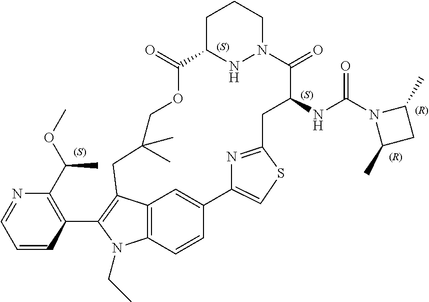 |
| DA 194 | 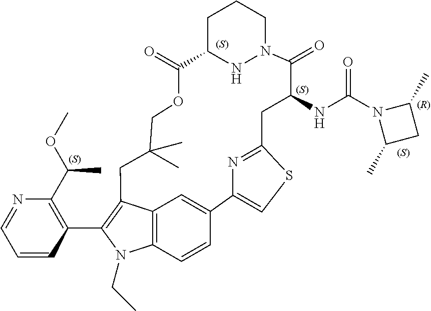 |
| DA 195 | 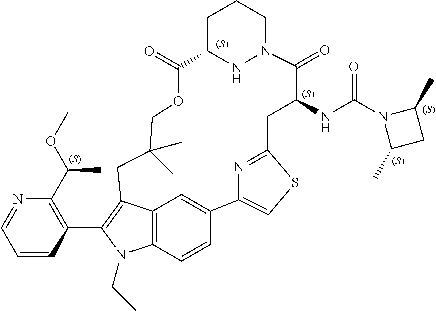 |
| DA 196 | 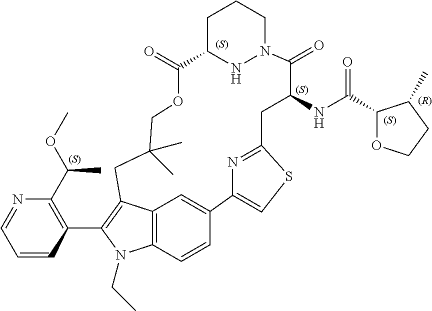 |
| DA 197 | 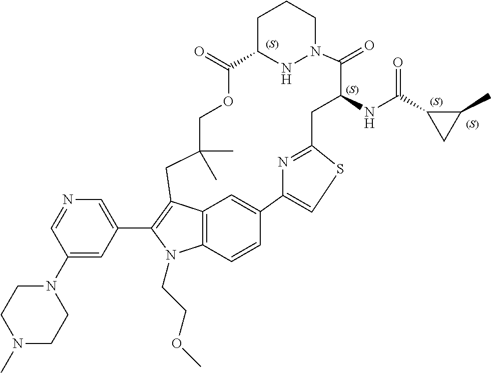 |
| DA 198 | 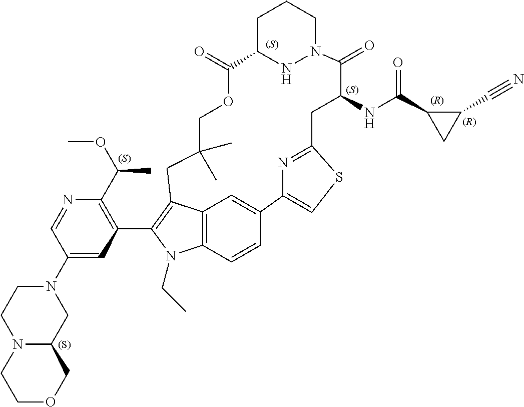 |
| DA 199 | 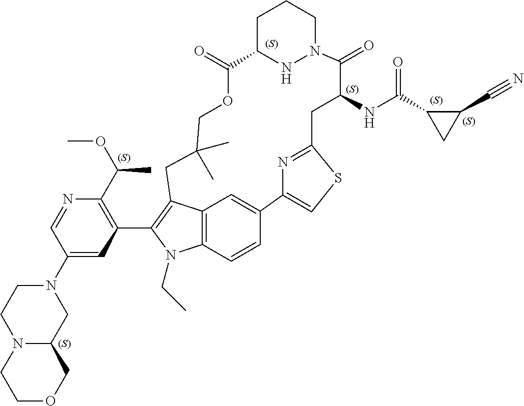 |
| DA 200 | 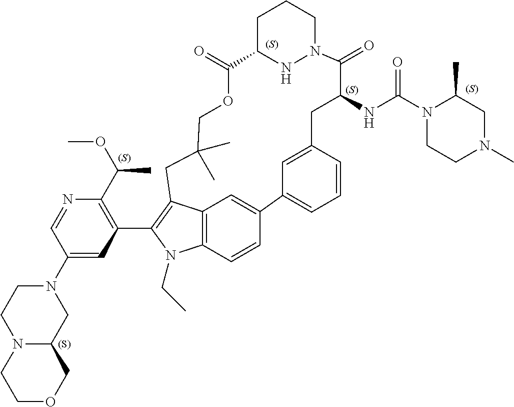 |
| DA 201 | 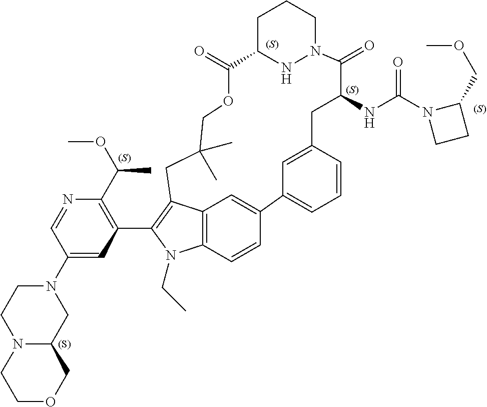 |
| DA 202 | 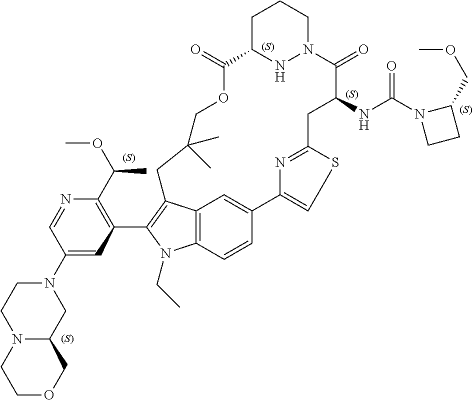 |
| DA 203 | 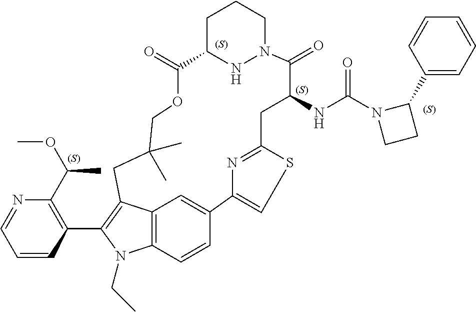 |
| DA 204 | 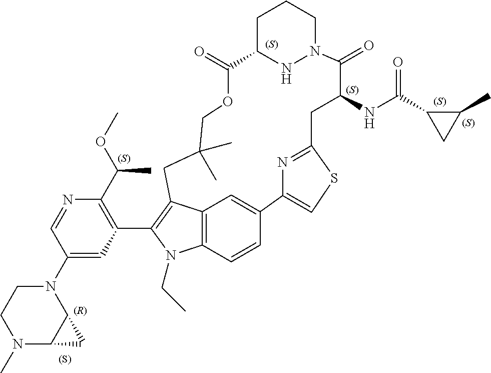 |
| DA 205 | 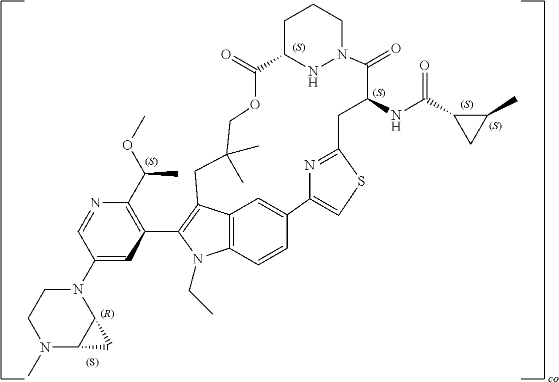 |
| DA 206 | 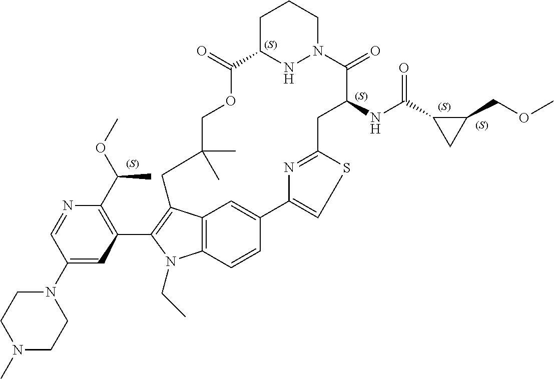 |
| DA 207 | 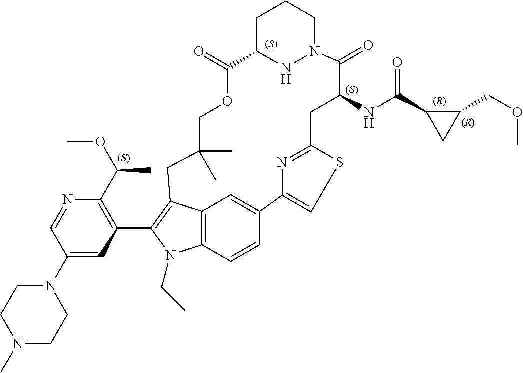 |
| DA 208 | 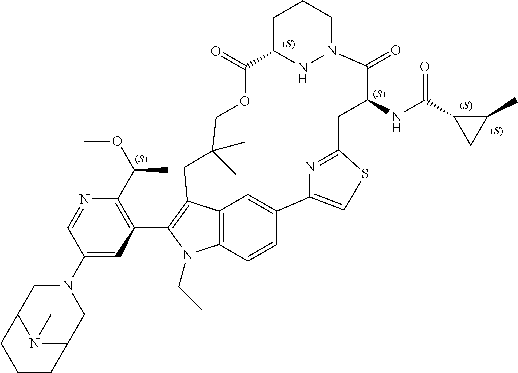 |
| DA 209 | 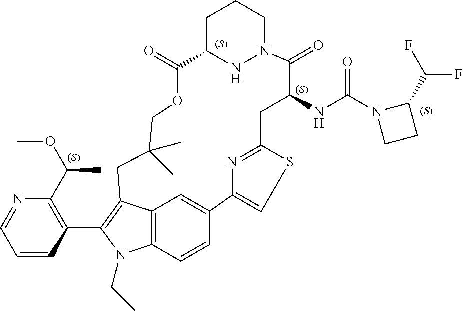 |
| DA 210 | 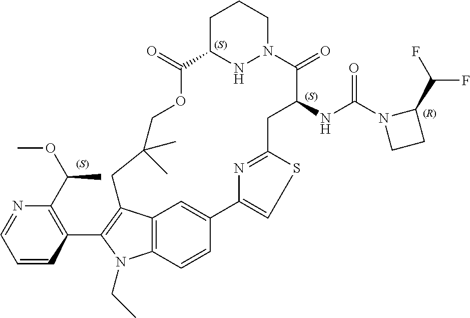 |
| DA 211 | 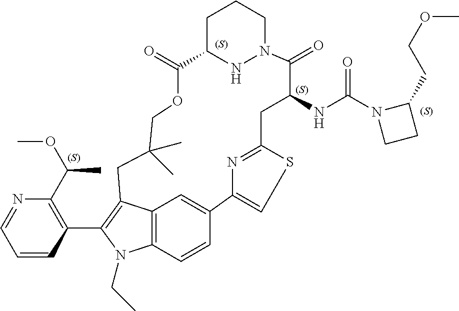 |
| DA 212 | 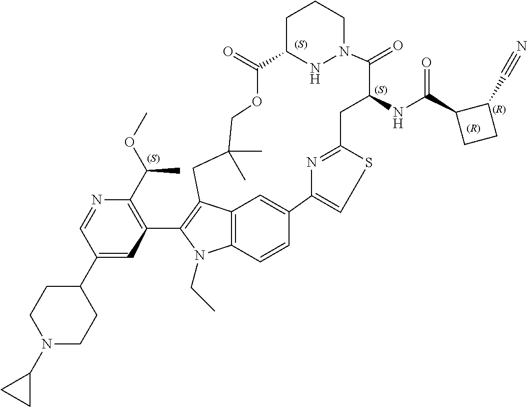 |
| DA 213 | 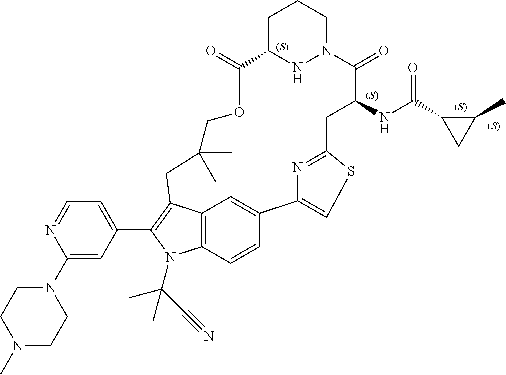 |
| DA 214 | 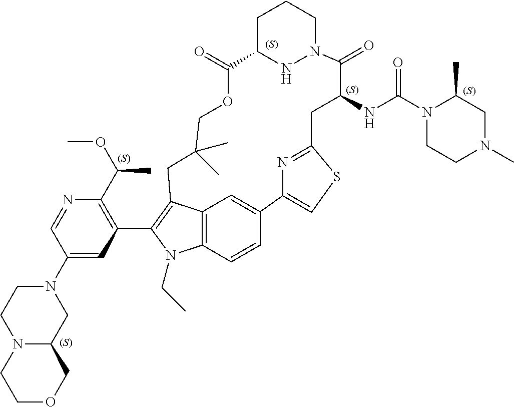 |
| DA 215 | 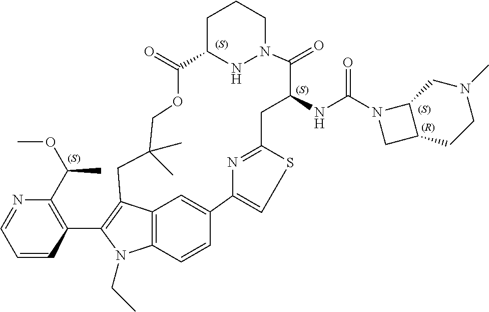 |
| DA 216 | 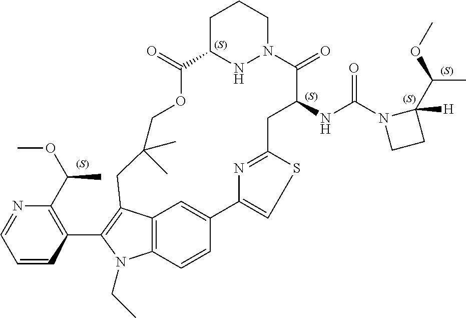 |
| DA 217 | 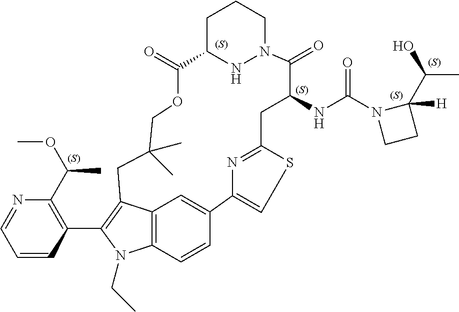 |
| DA 218 | 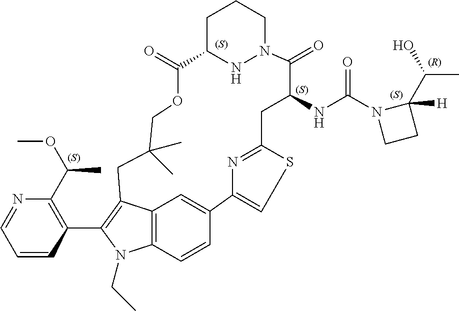 |
| DA 219 | 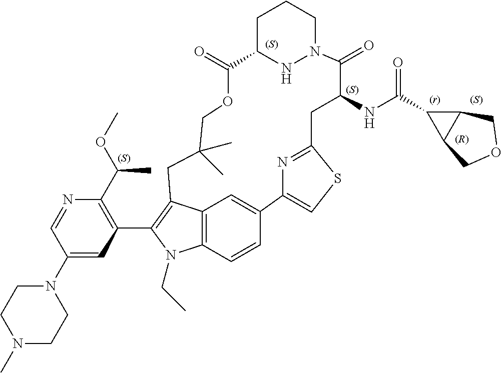 |
| DA 220 | 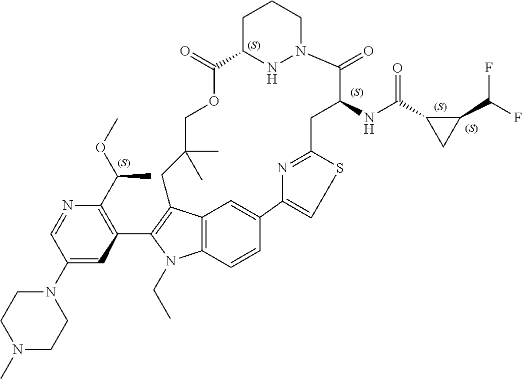 |
| DA 221 | 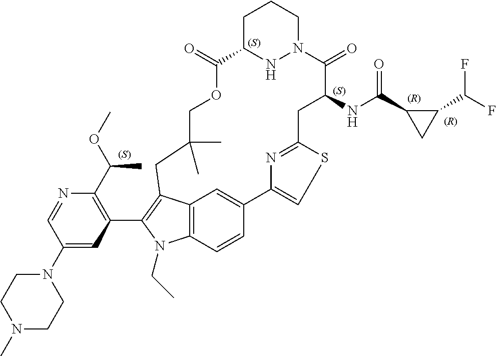 |
| DA 222 | 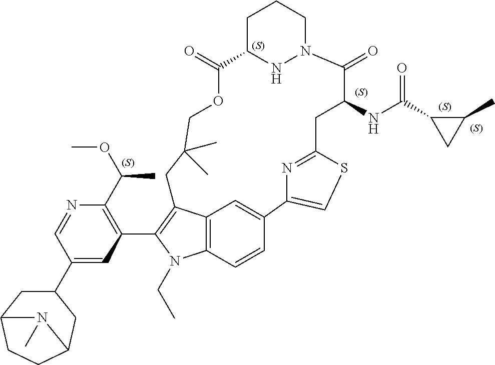 |
| DA 223 | 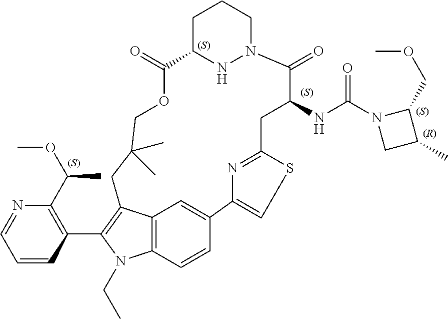 |
| DA 224 | 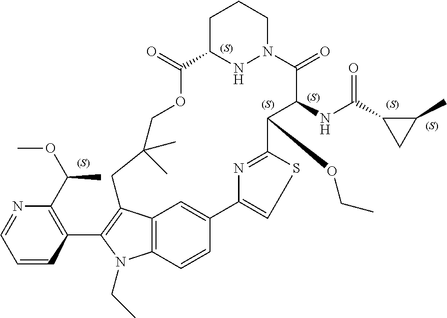 |
| DA 225 | 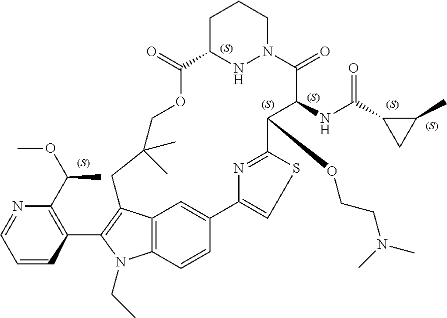 |
| DA 226 | 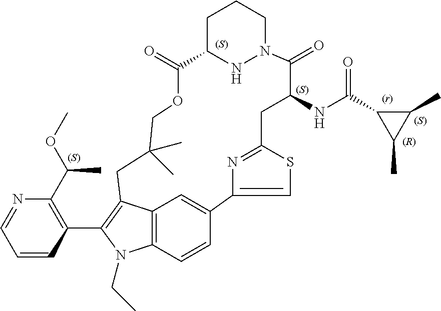 |
| DA 227 | 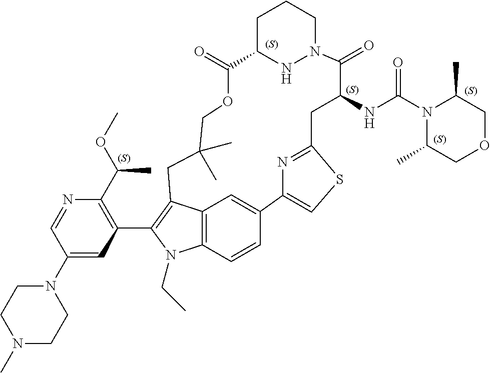 |
| DA 228 | 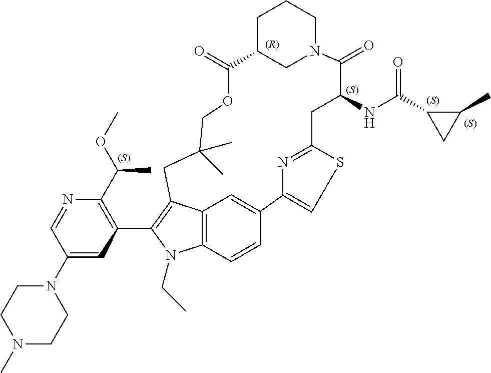 |
| DA 229 | 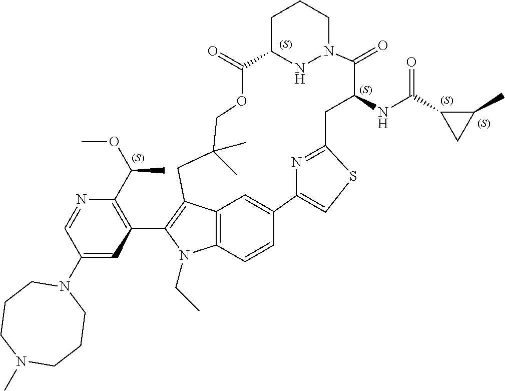 |
| DA 230 | 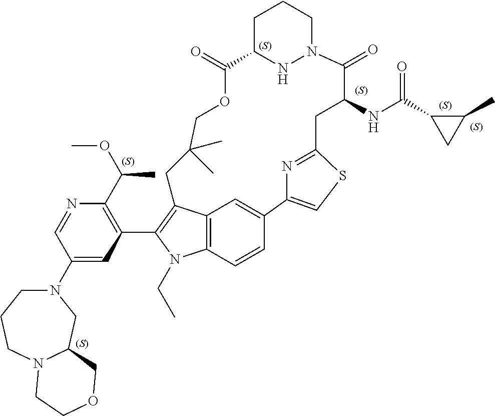 |
| DA 231 | 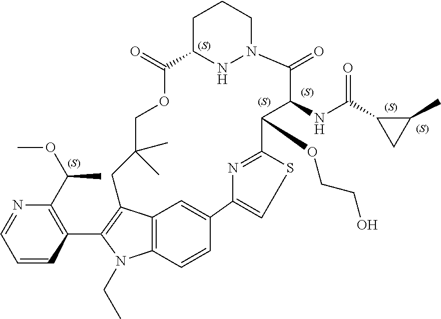 |
| DA 232 | 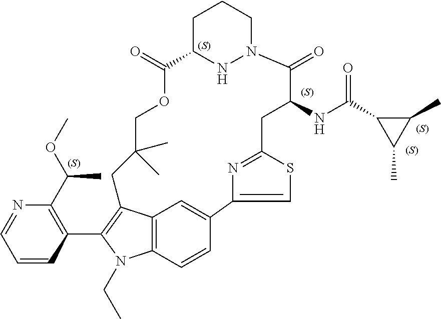 |
| DA 233 | 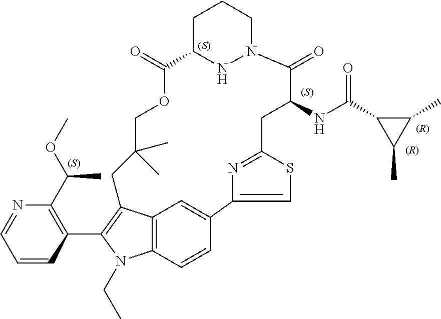 |
| DA 234 | 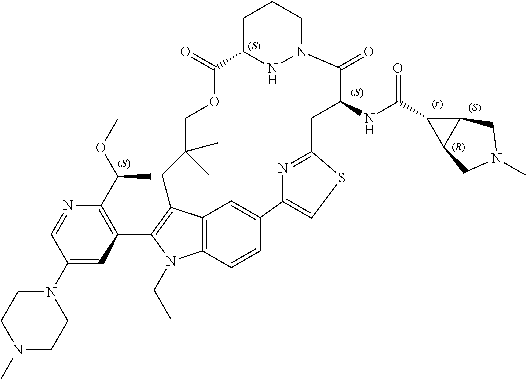 |
| DA 235 | 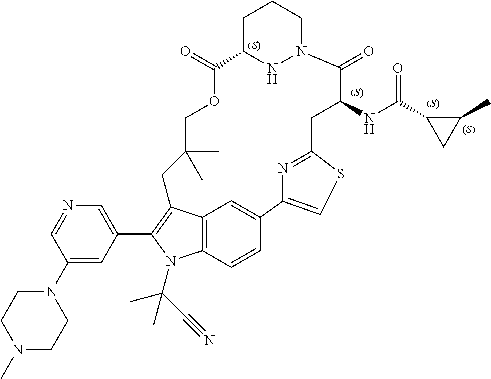 |
| DA 236 | 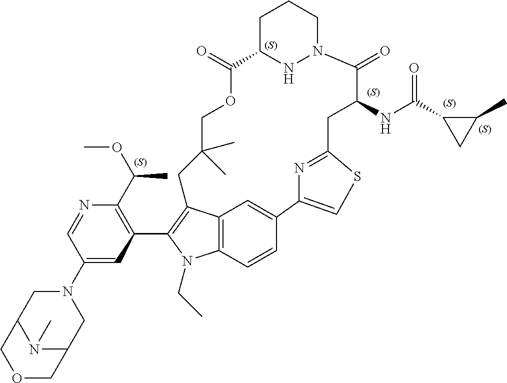 |
| DA 237 | 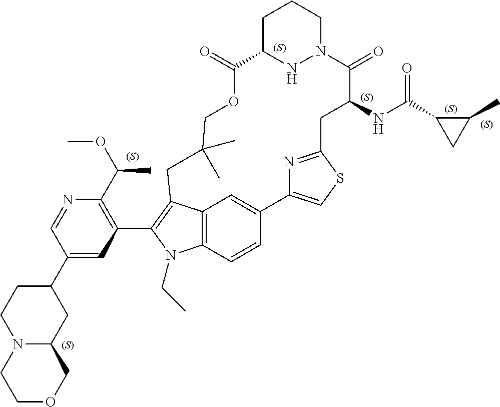 |
| DA 238 | 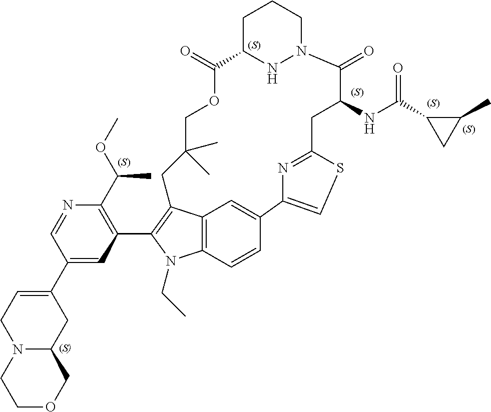 |
| DA 239 | 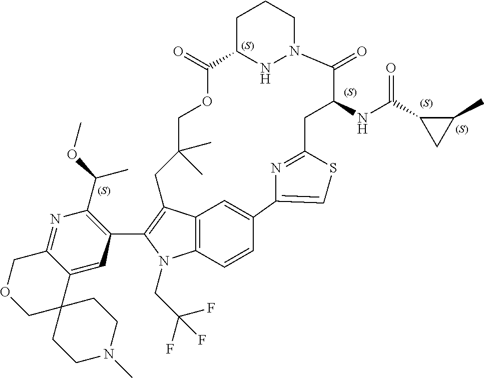 |
| DA 240 | 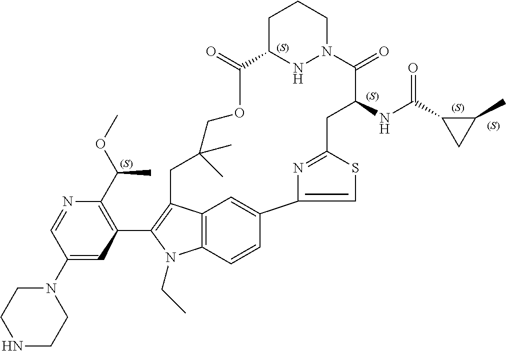 |
| DA 241 | 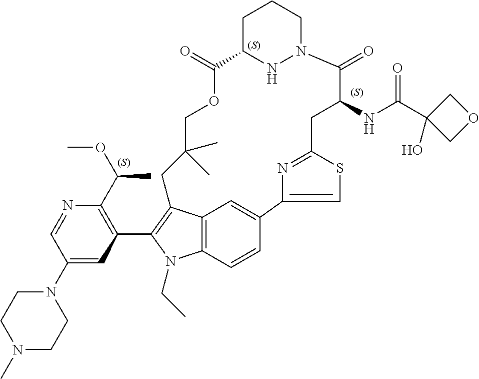 |
| DA 242 | 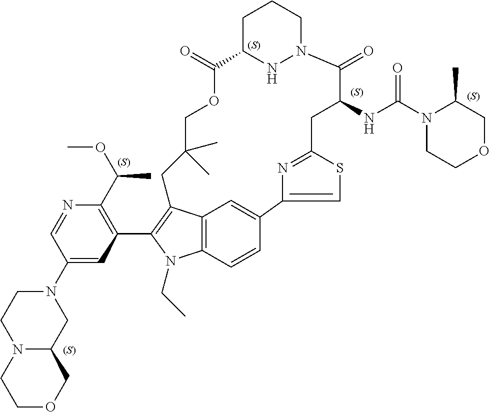 |
| DA 243 | 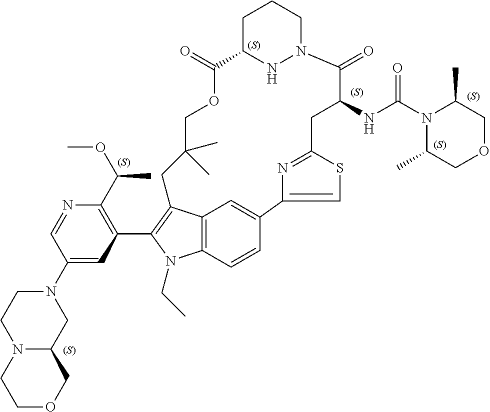 |
| DA 244 | 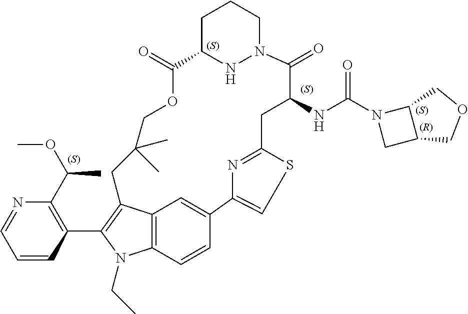 |
| DA 245 | 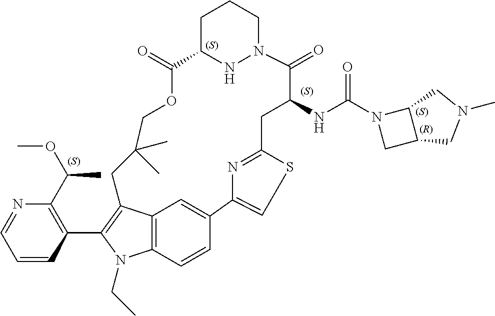 |
| DA 246 | 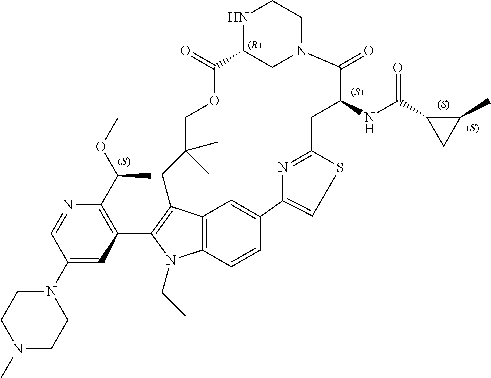 |
| DA 247 | 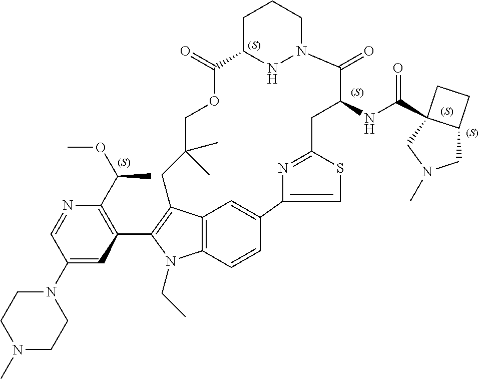 |
| DA 248 | 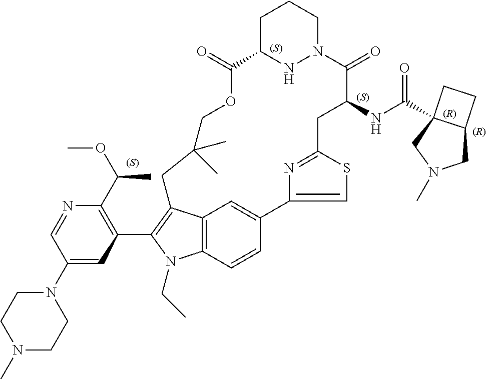 |
| DA 249 | 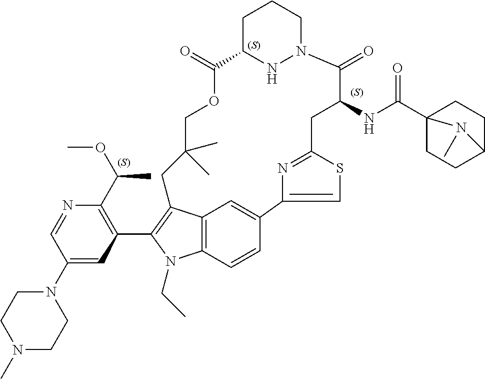 |
| DA 250 | 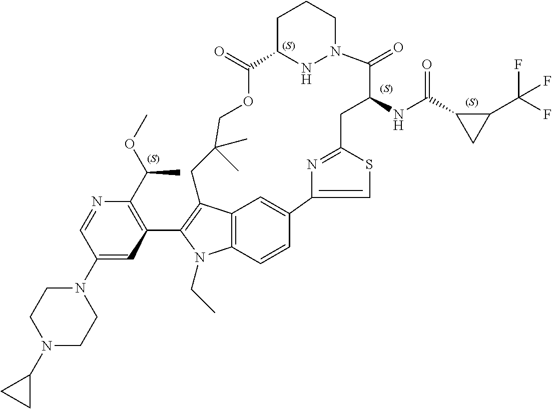 |
| DA 251 | 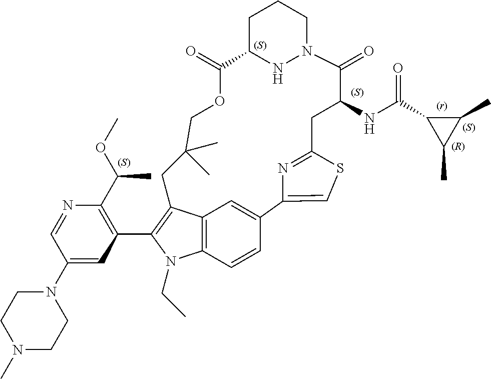 |
| DA 252 | 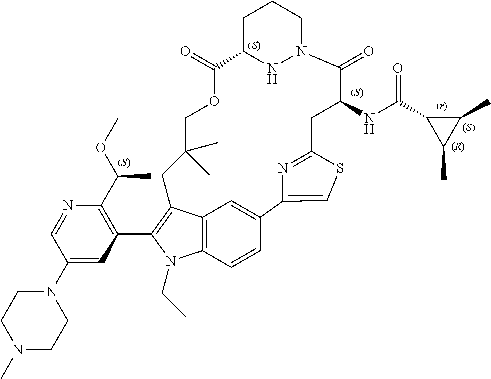 |
| DA 253 | 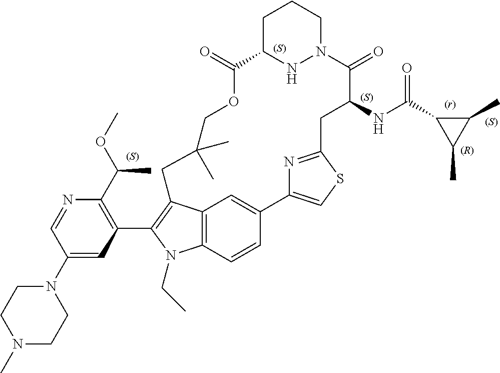 |
| DA 254 | 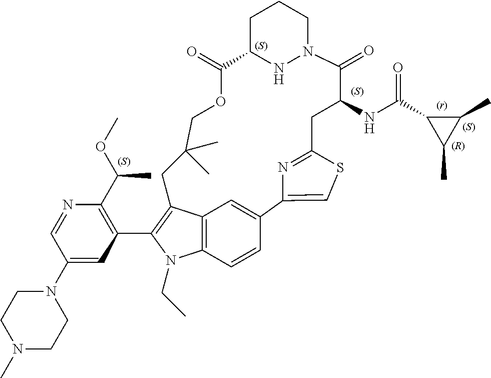 |
| DA 255 | 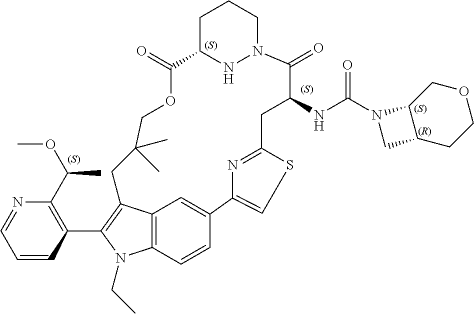 |
| DA 256 | 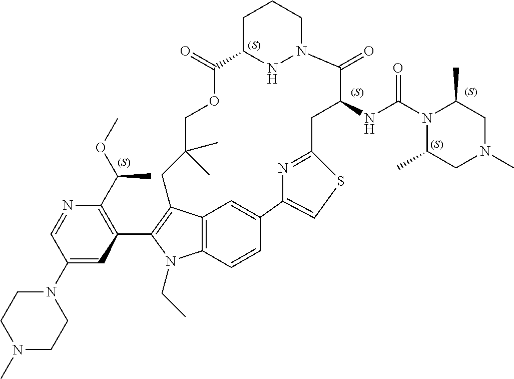 |
| DA 257 | 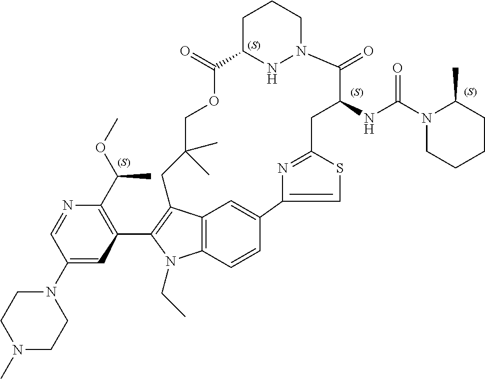 |
| DA 258 | 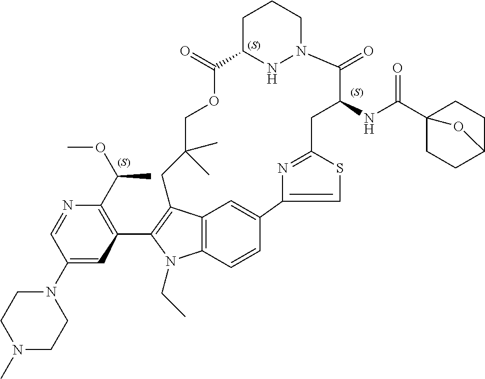 |
| DA 259 | 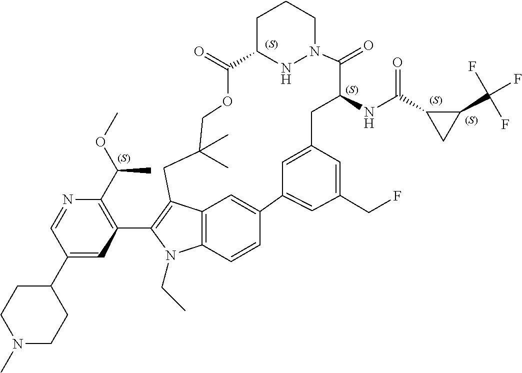 |
| DA 260 | 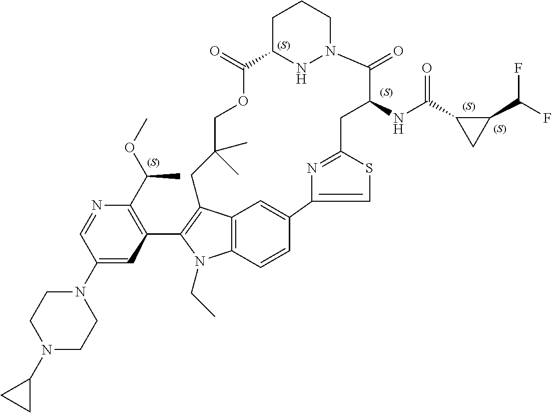 |
| DA 261 | 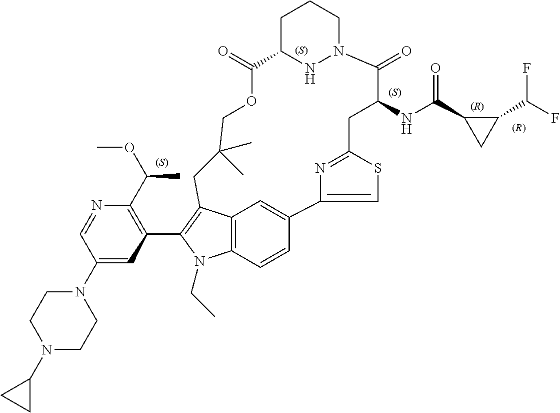 |
| DA 262 | 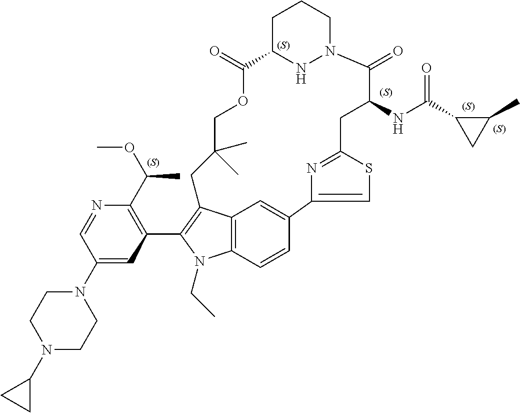 |
| DA 263 | 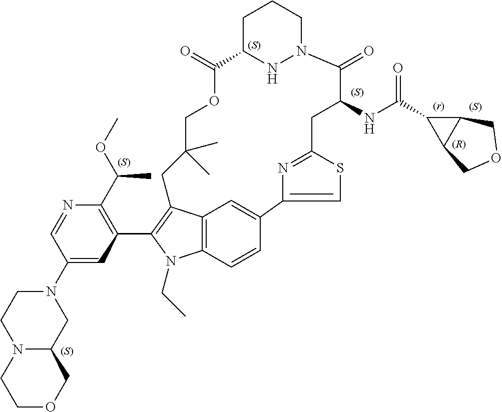 |
| DA 264 | 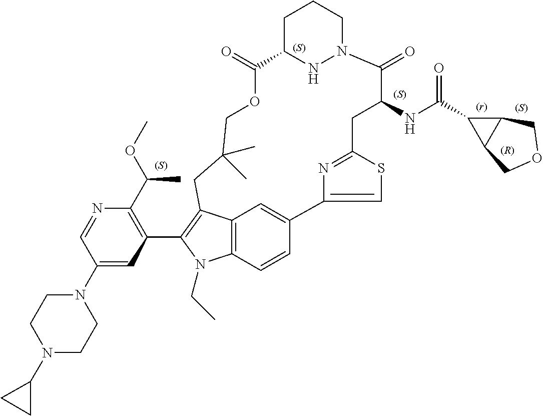 |
| DA 265 | 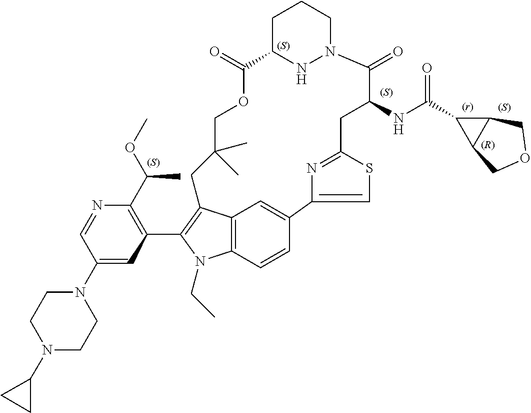 |
| DA 266 | 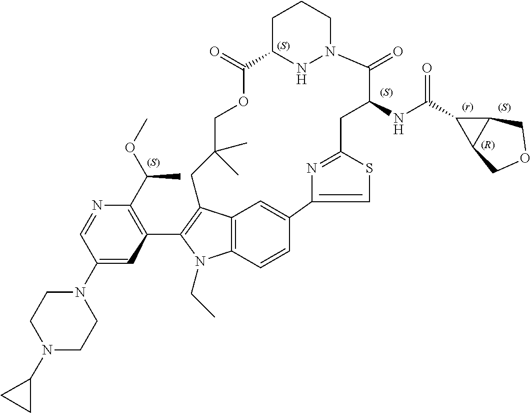 |
| DA 267 | 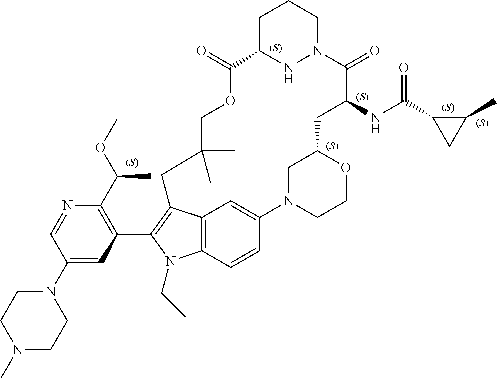 |
| DA 268 | 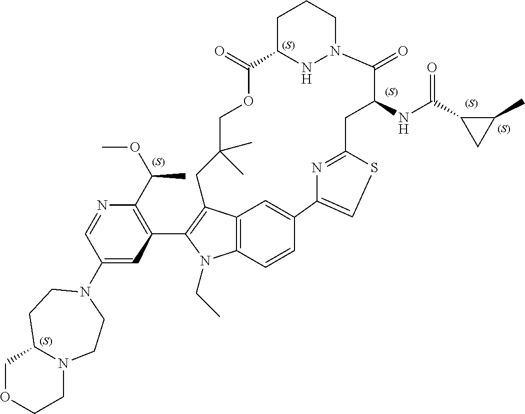 |
| DA 269 | 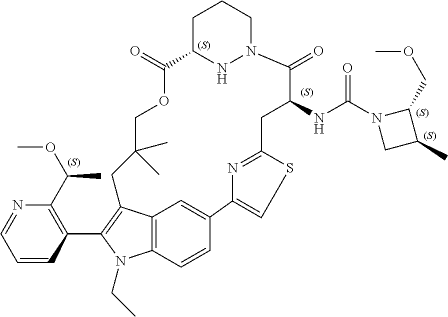 |
| DA 270 | 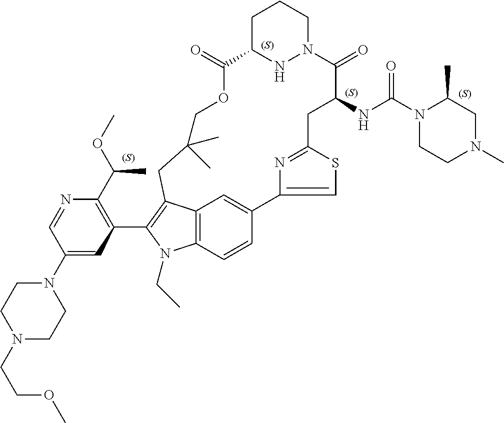 |
| DA 271 | 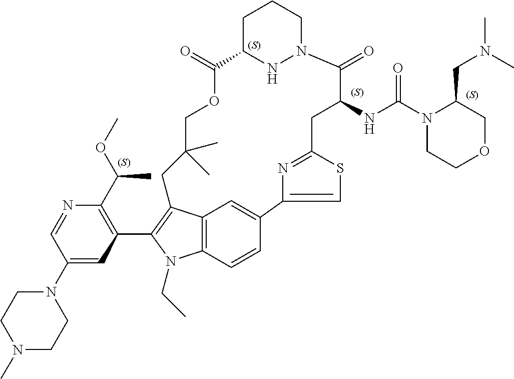 |
| DA 272 | 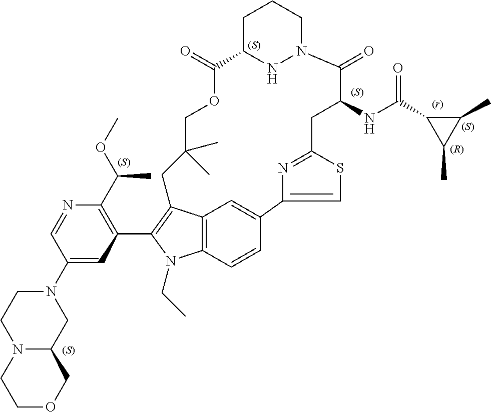 |
| DA 273 | 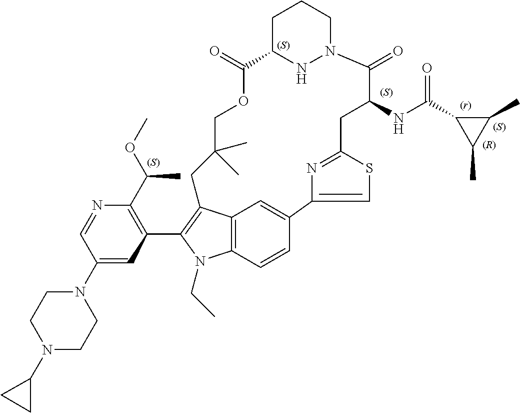 |
| DA 274 | 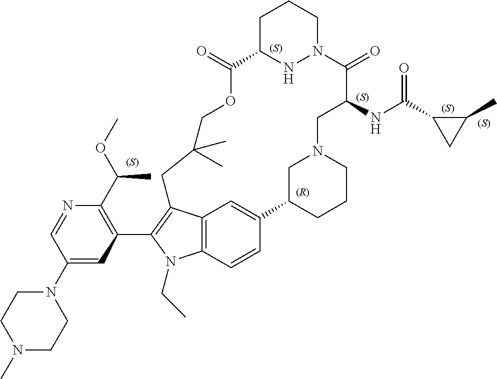 |
| DA 275 | 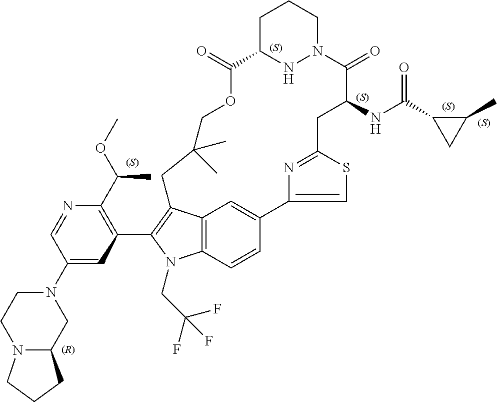 |
| DA 276 | 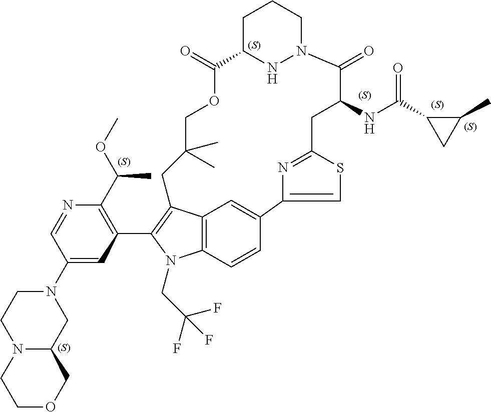 |
| DA 277 | 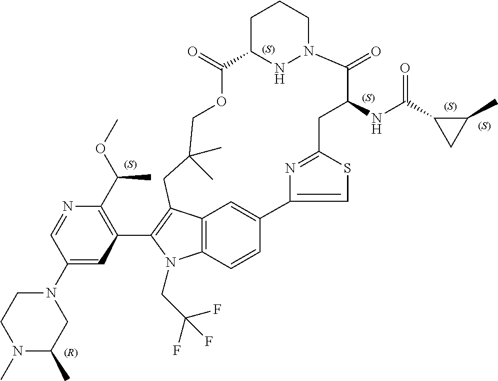 |
| DA 278 | 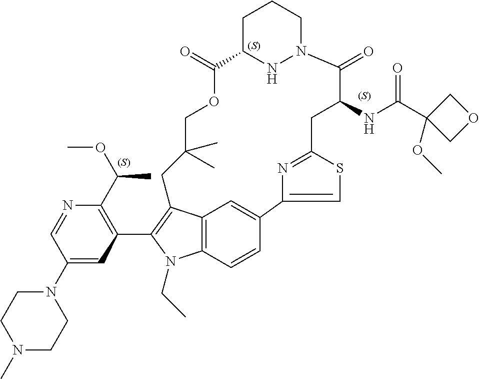 |
| DA 279 | 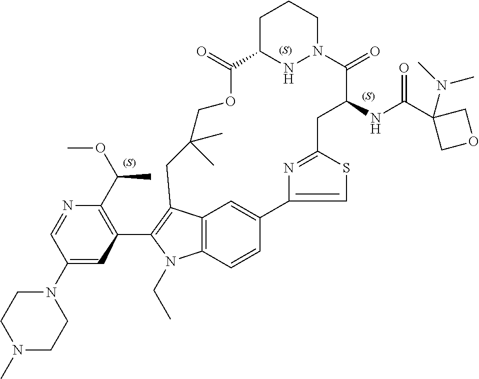 |
| DA 280 | 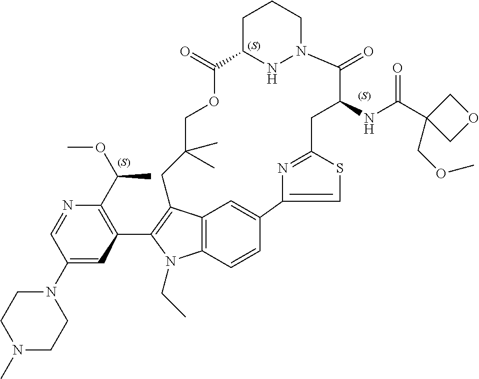 |
| DA 281 | 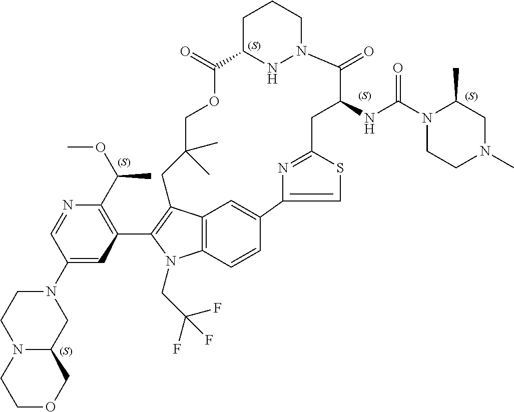 |
| DA 282 | 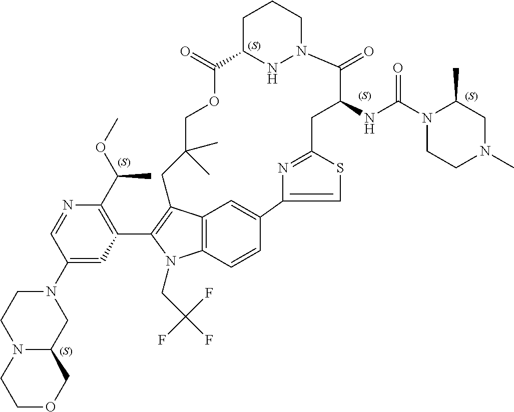 |
| DA 283 | 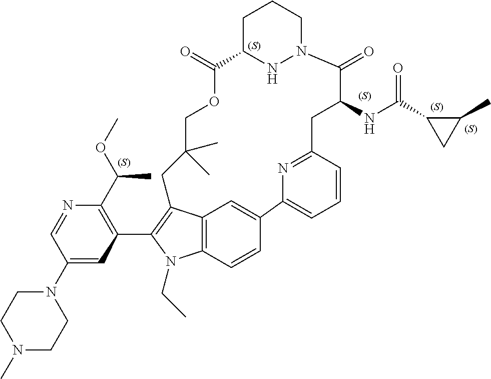 |
| DA 284 | 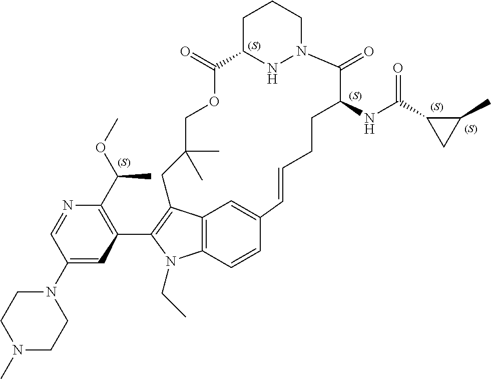 |
| DA 285 | 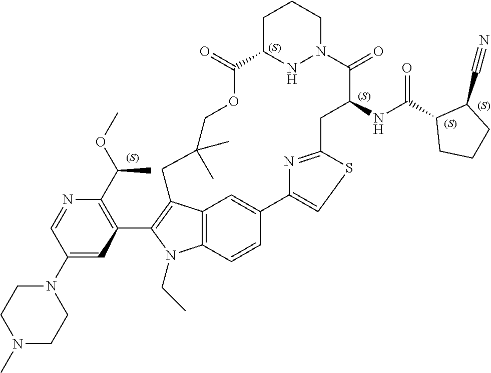 |
| DA 286 | 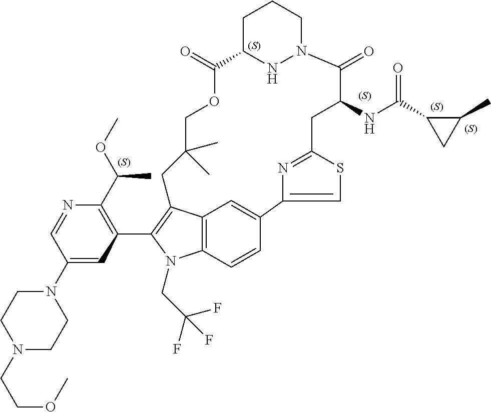 |
| DA 287 | 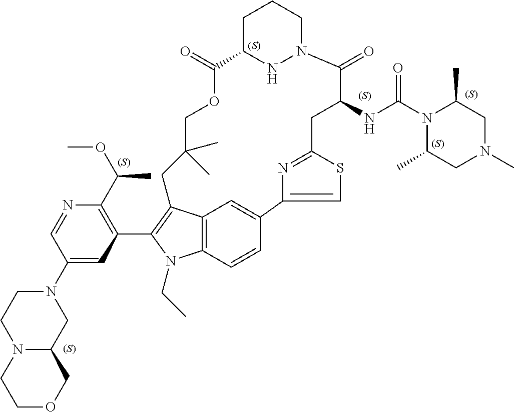 |
| DA 288 | 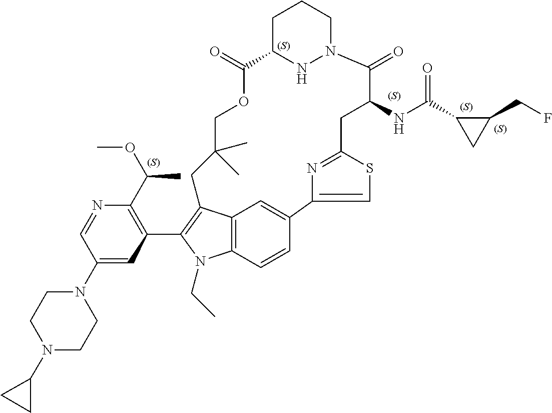 |
| DA 289 | 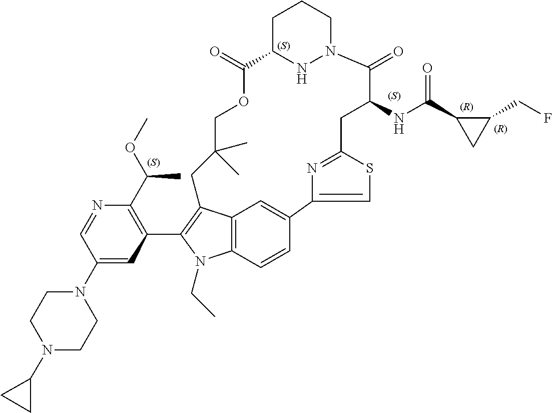 |
| DA 290 | 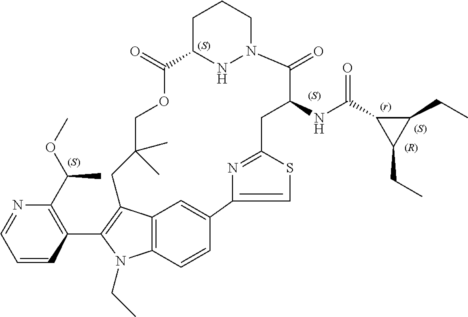 |
| DA 291 | 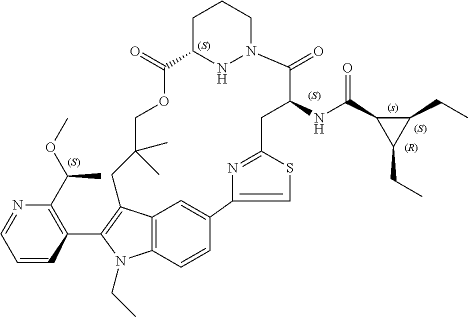 |
| DA 292 | 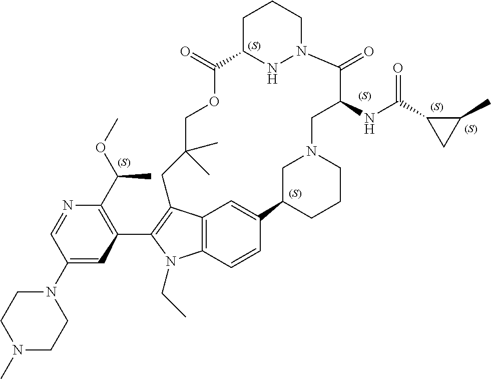 |
| DA 293 | 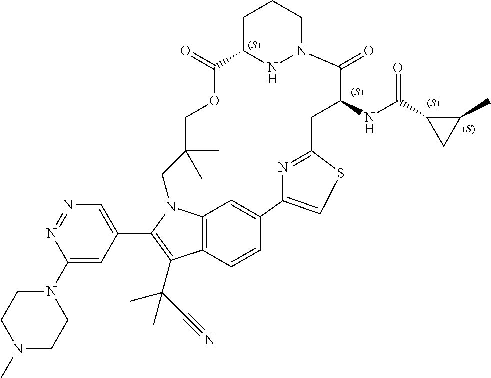 |
| DA 294 | 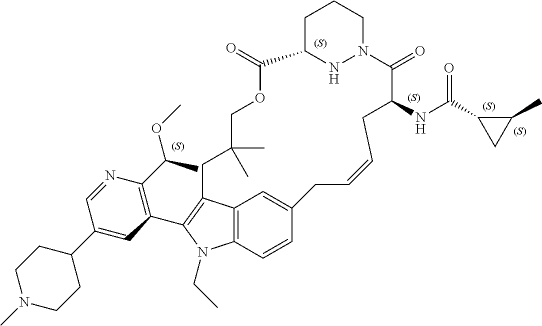 |
| DA 295 | 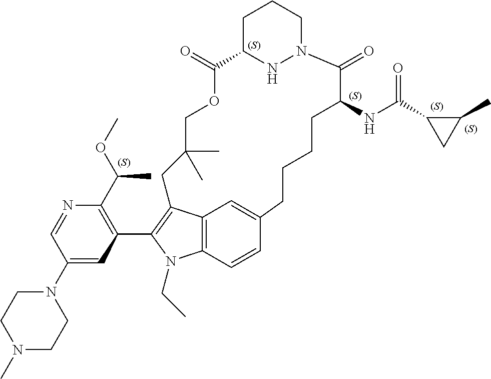 |
| DA 296 | 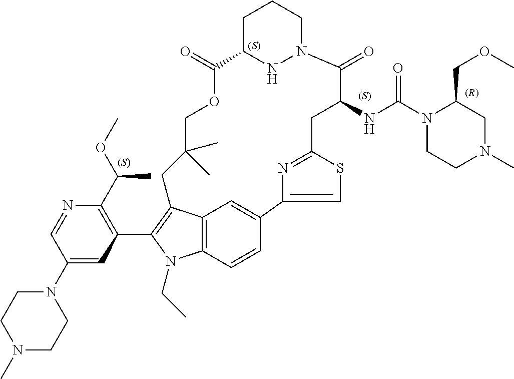 |
| DA 297 | 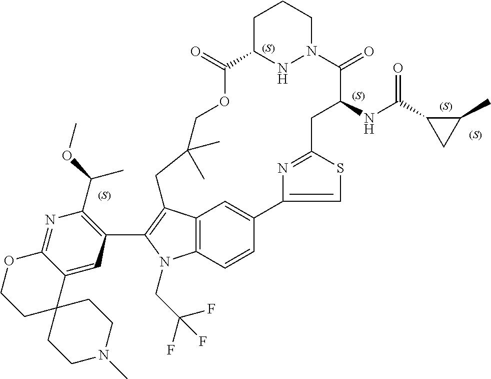 |
| DA 298 | 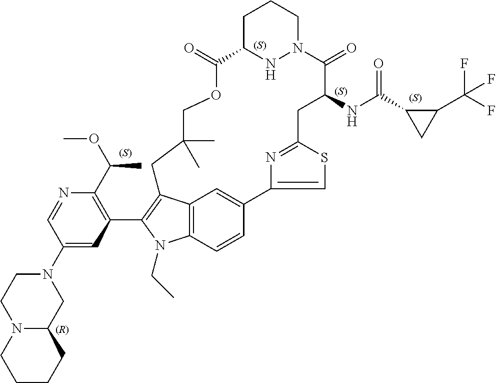 |
| DA 299 | 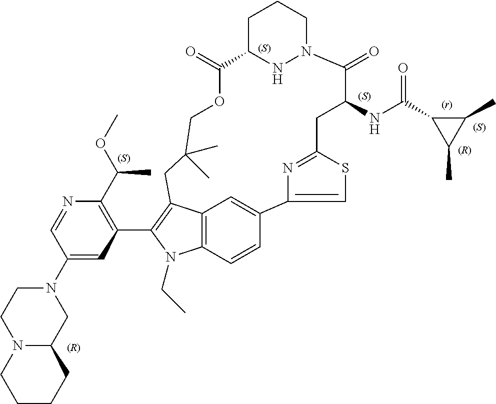 |
| DA 300 | 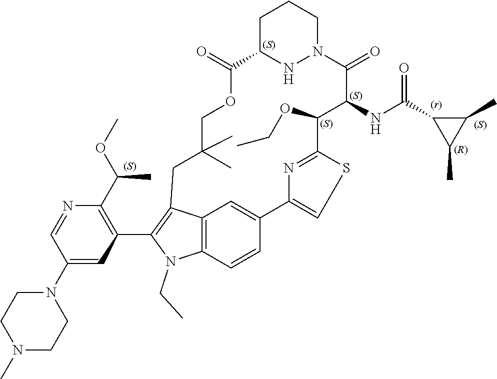 |
| DA 301 | 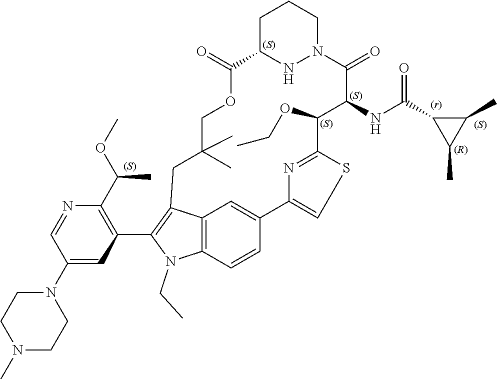 |
| DA 302 | 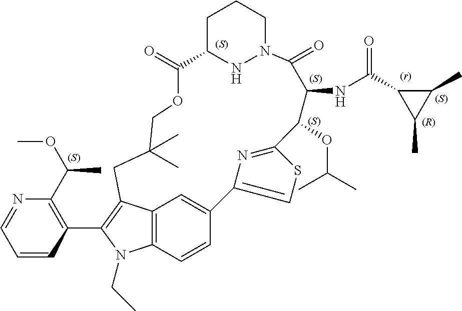 |
| DA 303 | 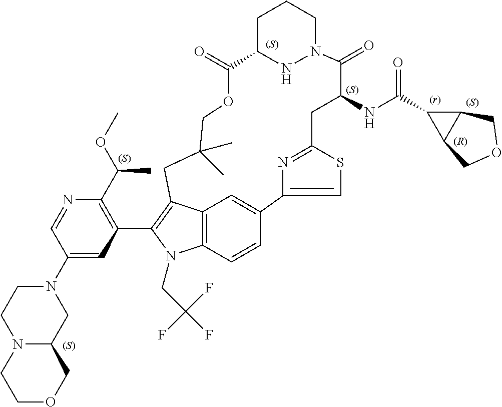 |
| DA 304 | 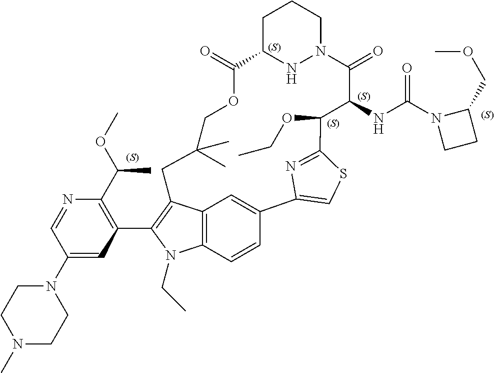 |
| DA 305 | 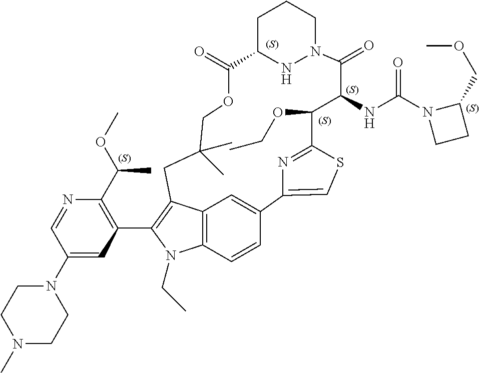 |
| DA 306 | 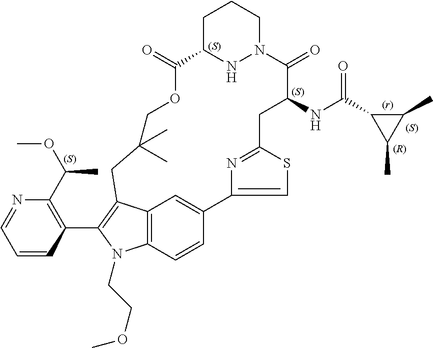 |
| DA 307 | 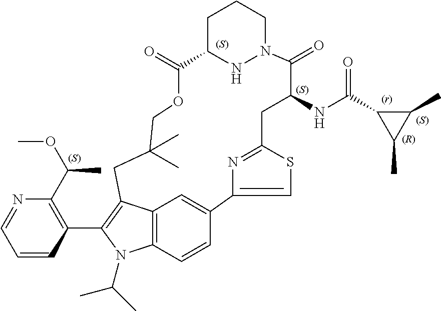 |
| DA 308 | 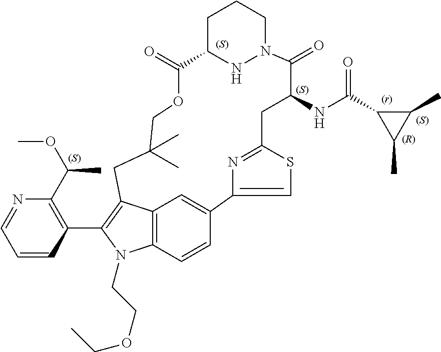 |
| DA 309 | 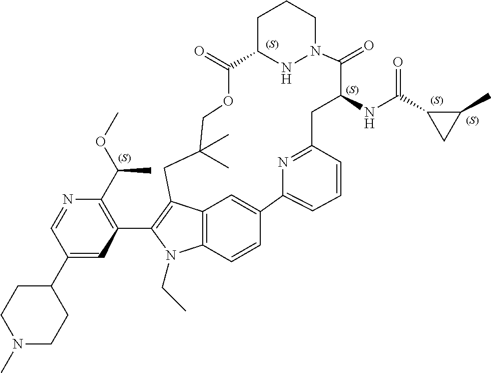 |
| DA 310 | 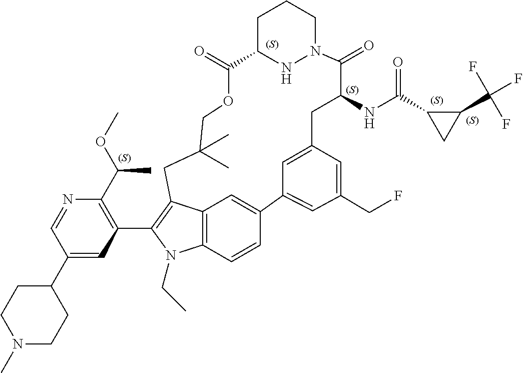 |
| DA 311 | 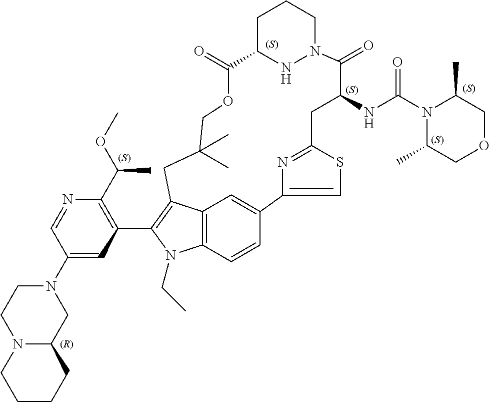 |
| DA 312 | 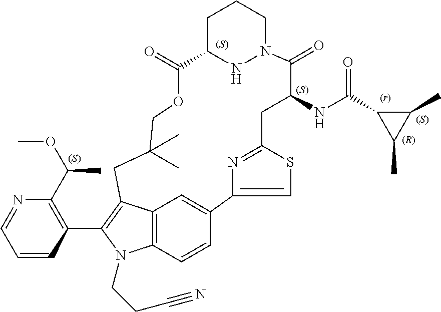 |
| DA 313 | 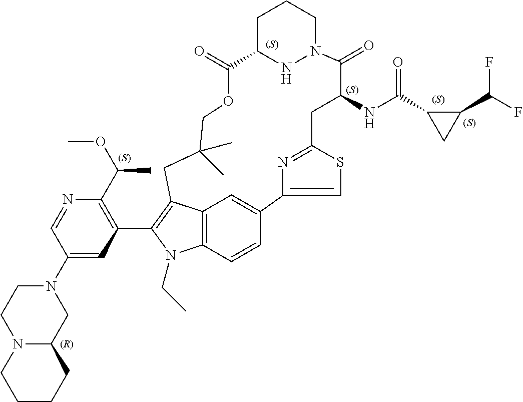 |
| DA 314 | 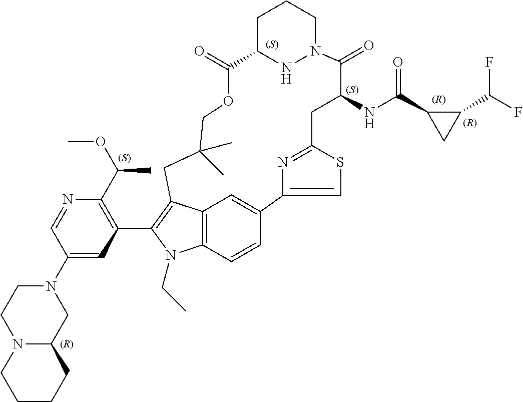 |
| DA 315 | 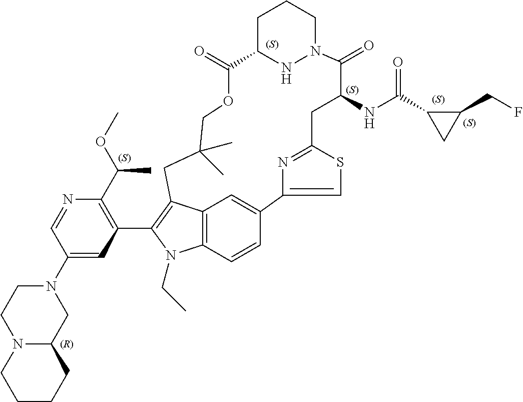 |
| DA 316 | 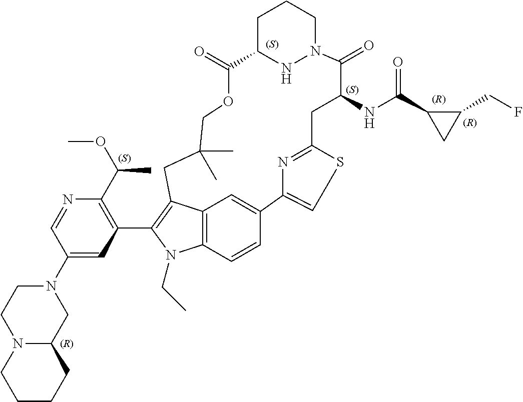 |
| DA 317 | 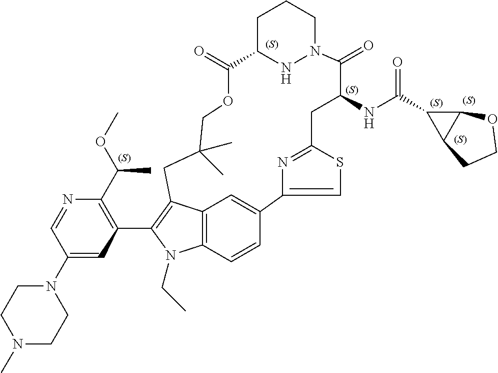 |
| DA 318 | 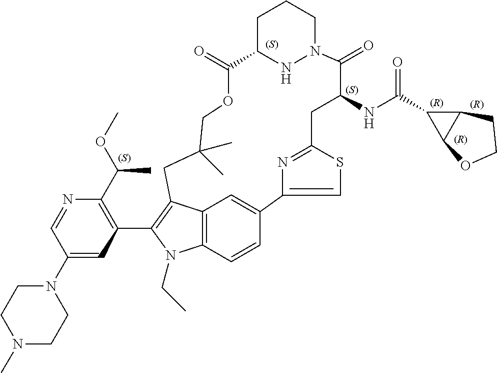 |
| DA 319 | 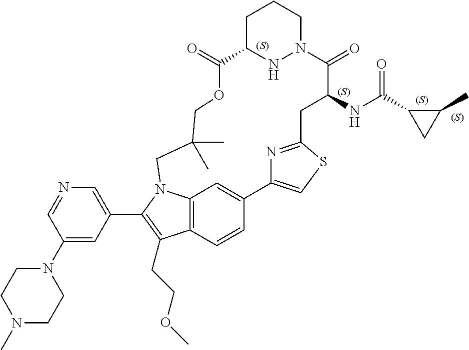 |
| DA 320 | 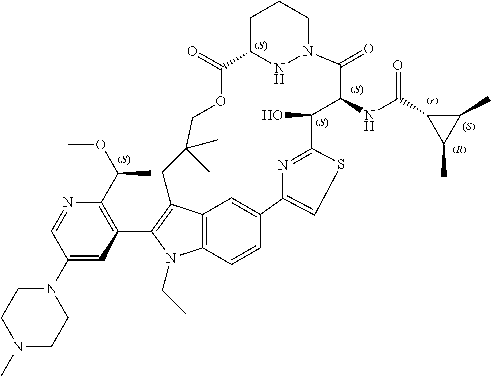 |
| DA 321 | 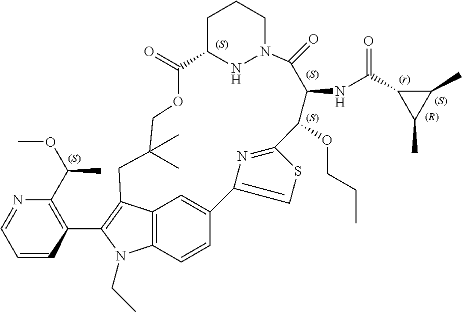 |
| DA 322 | 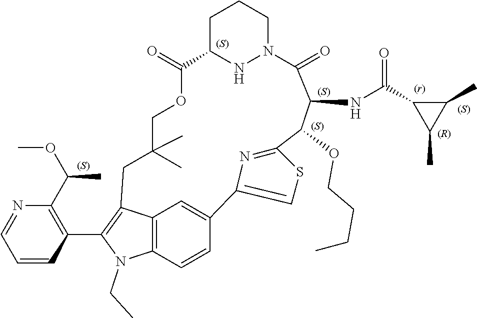 |
| DA 323 | 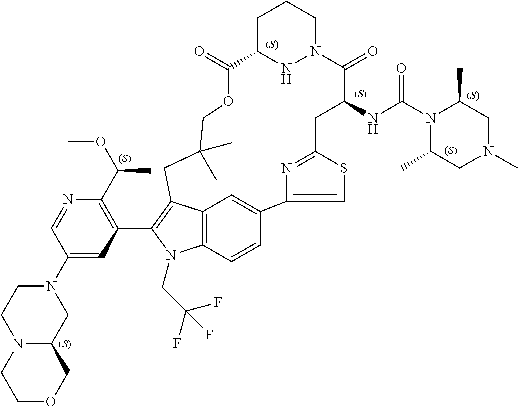 |
| DA 324 | 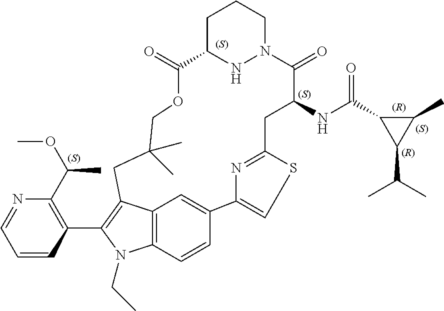 |
| DA 325 | 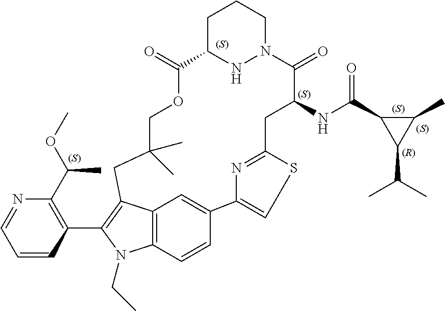 |
| DA 326 | 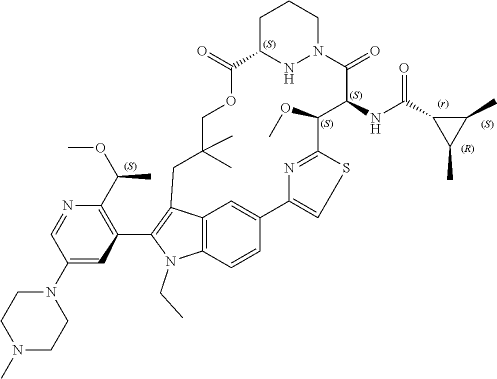 |
| DA 327 | 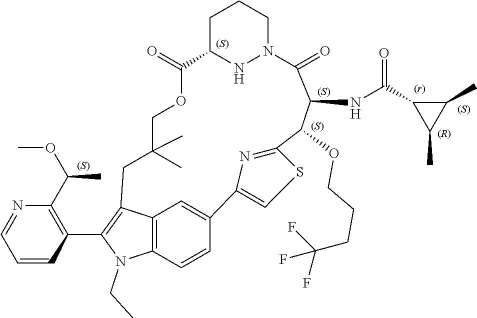 |
| DA 328 | 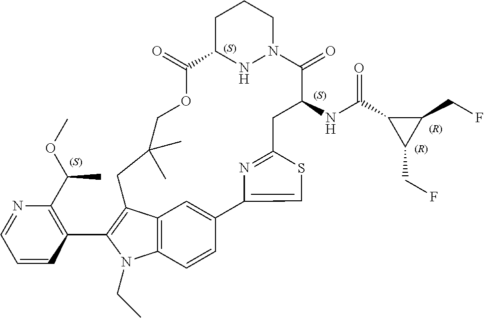 |
| DA 329 | 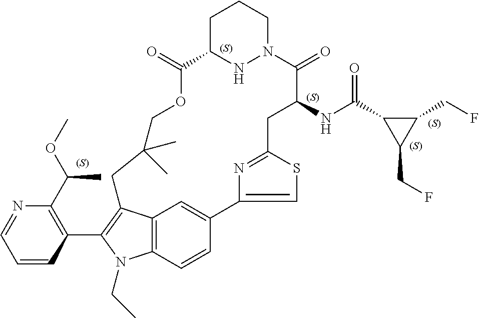 |
| DA 330 | 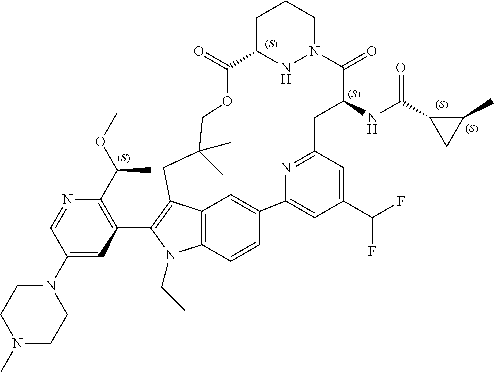 |
| DA 331 | 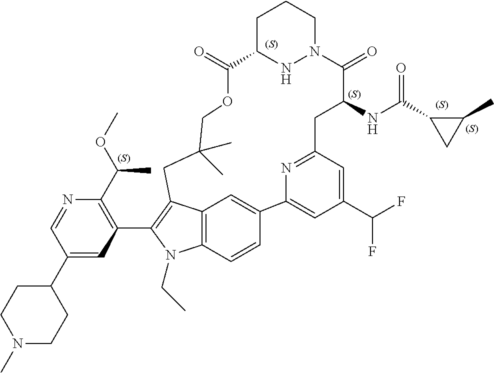 |
| DA 332 | 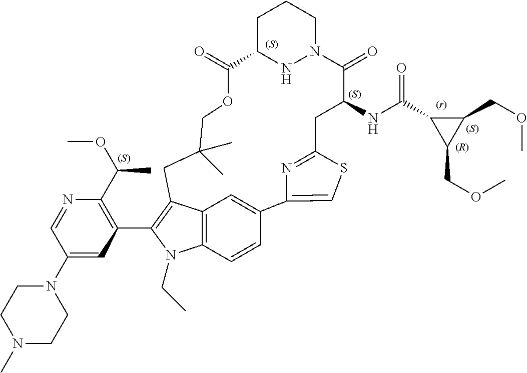 |
| DA 333 | 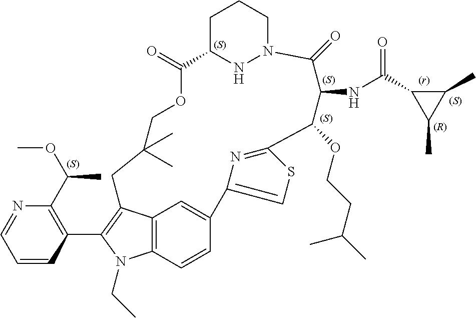 |
| DA 334 | 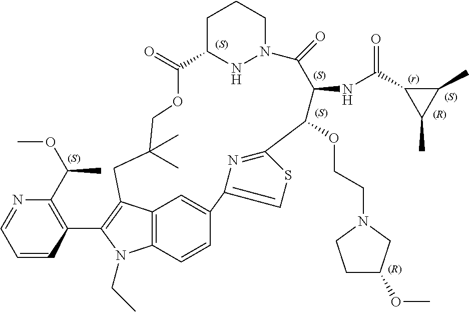 |
| DA 335 | 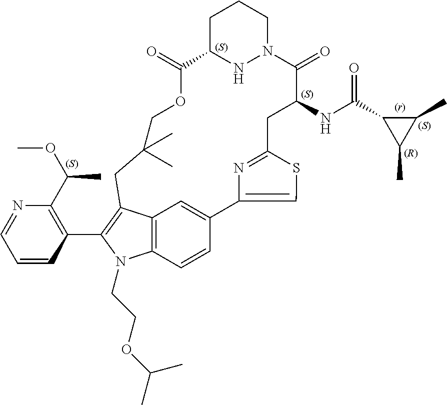 |
| DA 336 | 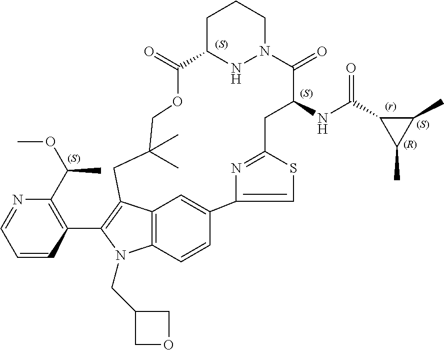 |
| DA 337 | 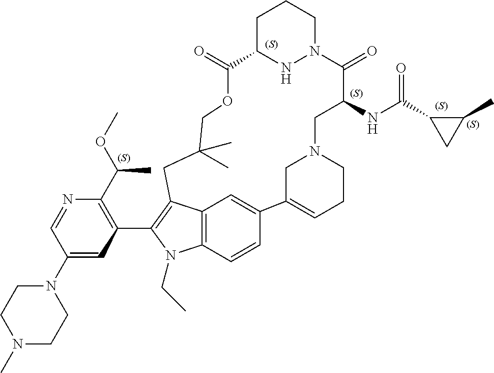 |
| DA 338 | 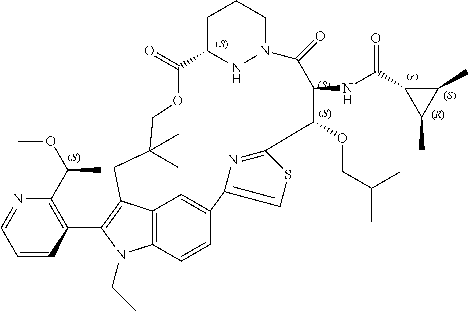 |
| DA 339 | 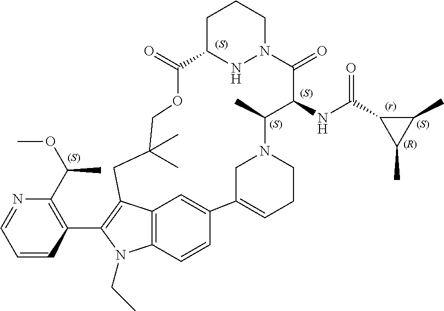 |
| DA 340 | 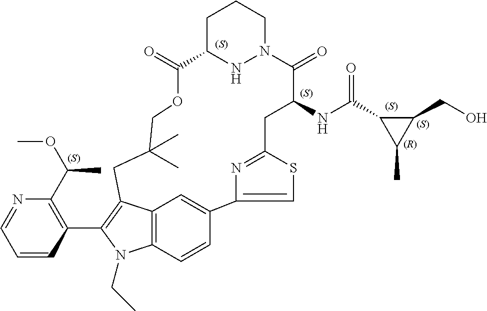 |
| DA 341 | 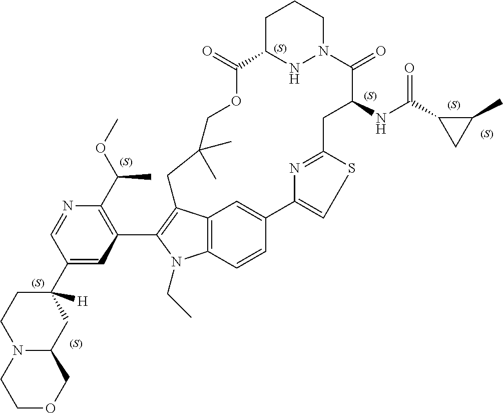 |
| DA 342 | 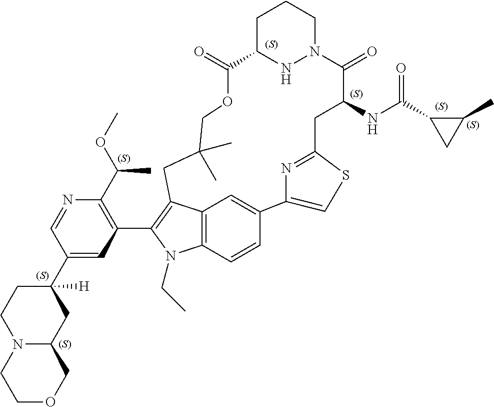 |
| DA 343 | 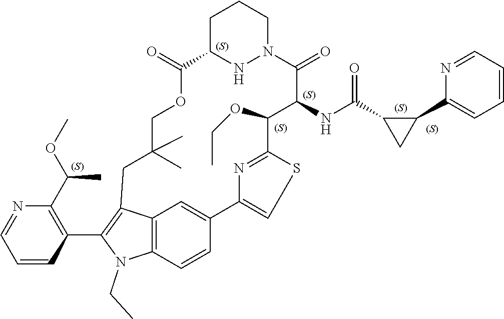 |
| DA 344 | 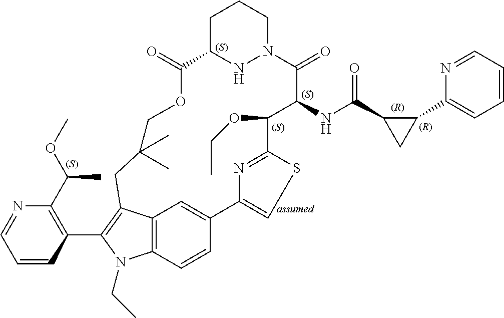 |
| DA 345 | 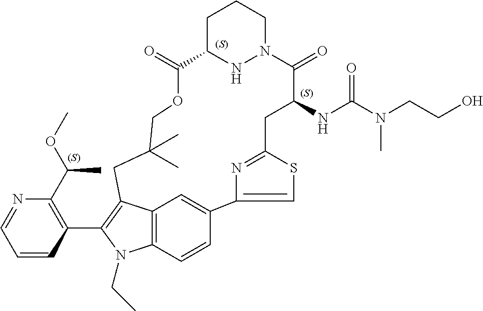 |
| DA 346 | 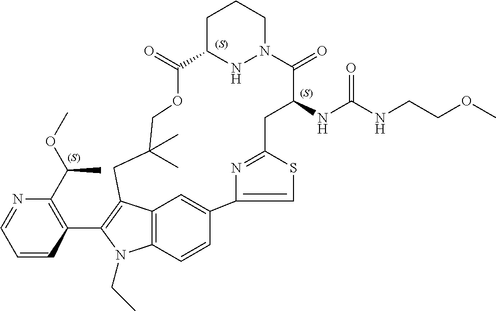 |
| DA 347 | 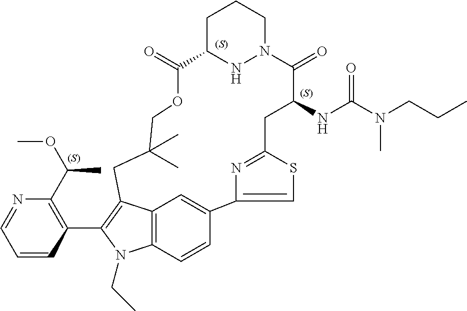 |
| DA 348 | 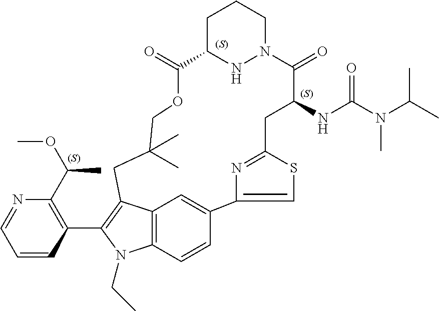 |
| DA 349 | 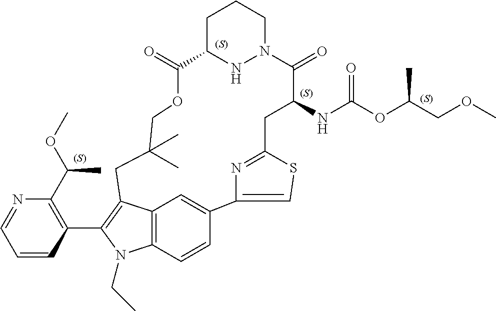 |
| DA 350 | 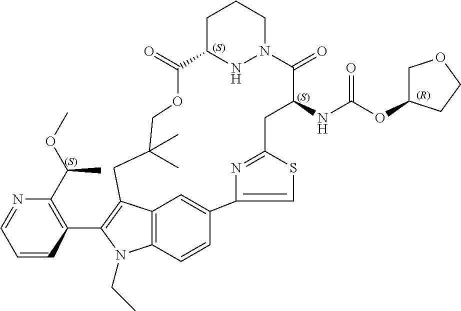 |
| DA 351 | 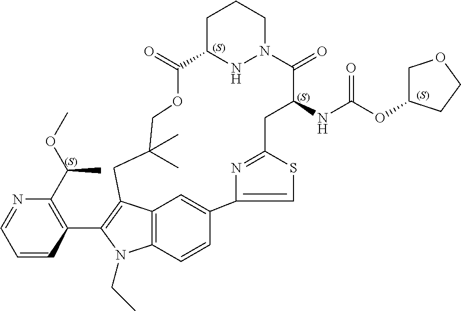 |
| DA 352 | 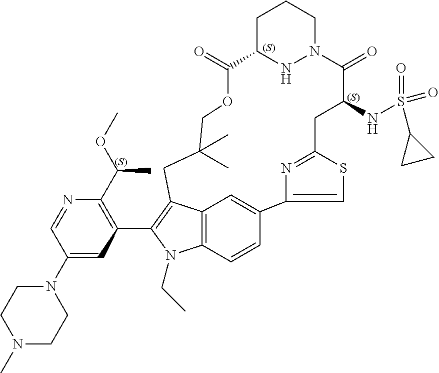 |

Note that some compounds are shown with bonds as flat or wedged.
In some instances, the relative stereochemistry of stereoisomers has been determined; in some instances, the absolute stereochemistry has been determined.
All stereoisomers of the compounds of the foregoing table are contemplated by the present invention.
In particular embodiments, an atropisomer of a compound of the foregoing table is contemplated.
Any compound shown in brackets indicates that the compound is a diastereomer, and the absolute stereochemistry of such diastereomer may not be known.

In some embodiments, the RAS(MULTI) inhibitor is selected from Table D1b, or a pharmaceutically acceptable salt or stereoisomer thereof. In some embodiments, the RAS(MULTI) inhibitor is selected from Table D1b, or a pharmaceutically acceptable salt or atropisomer thereof.

TABLE D1b
Certain Compounds of the Present Invention

| Ex. | Structure |
|---|---|
| DA 354 | 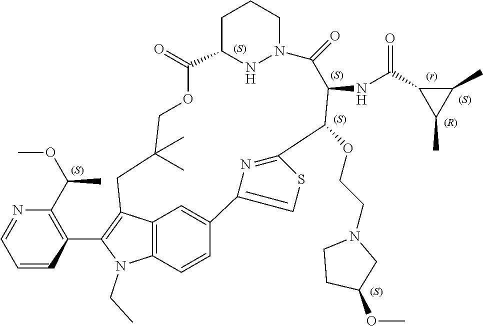 |
| DA 355 | 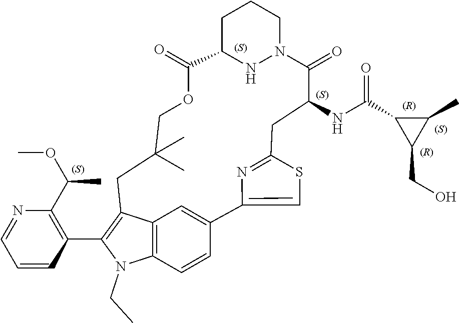 |
| DA 356 | 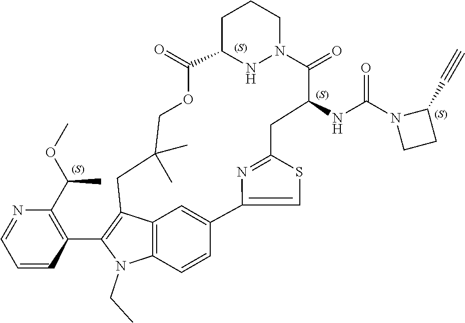 |
| DA 357 | 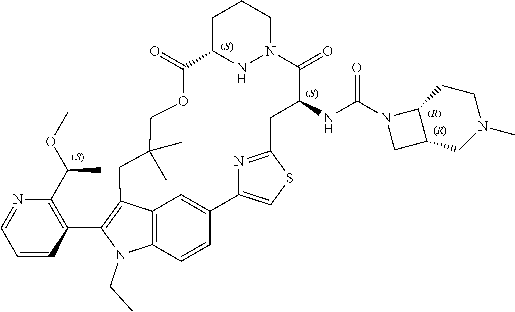 |
| DA 358 | 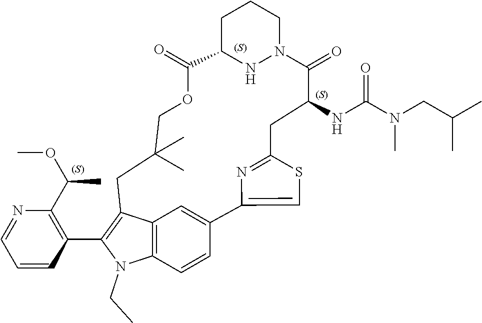 |
| DA 359 | 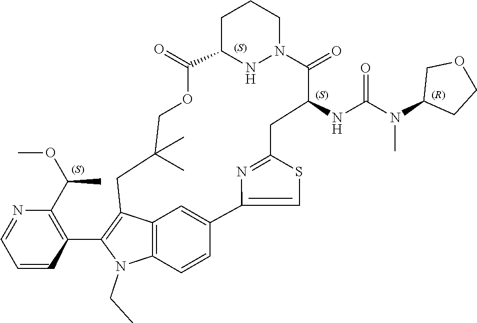 |
| DA 360 | 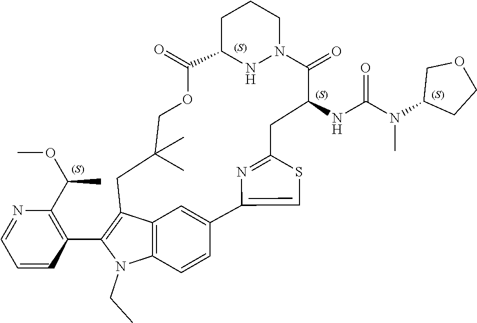 |
| DA 361 | 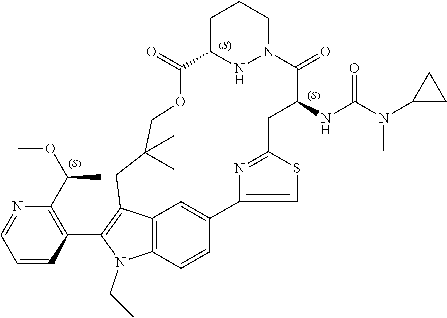 |
| DA 362 | 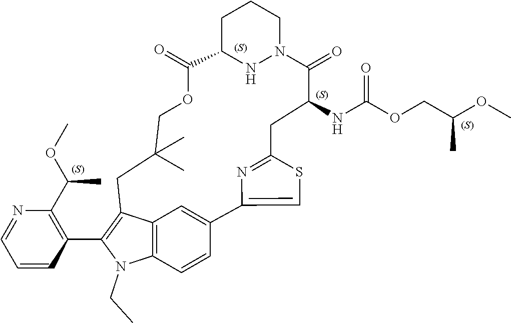 |
| DA 363 | 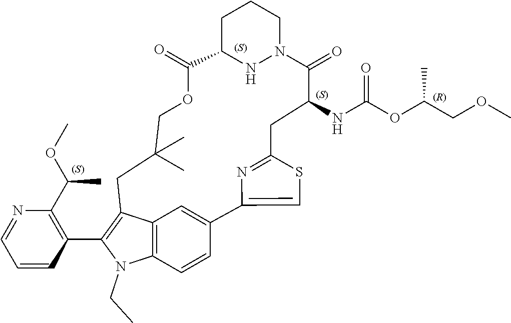 |
| DA 364 | 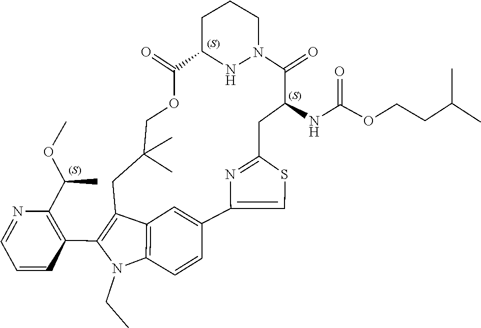 |
| DA 365 | 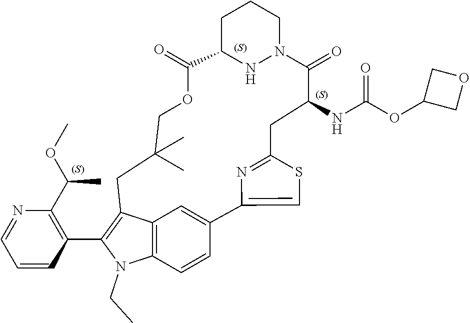 |
| DA 366 | 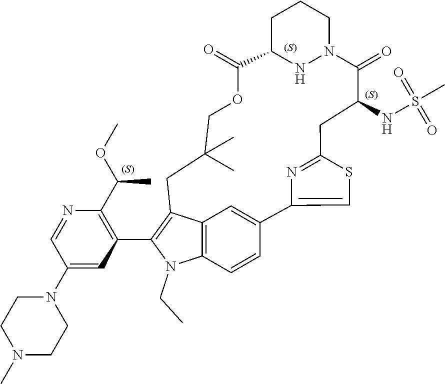 |
| DA 367 | 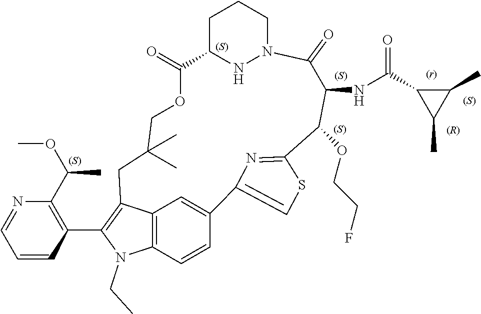 |
| DA 368 | 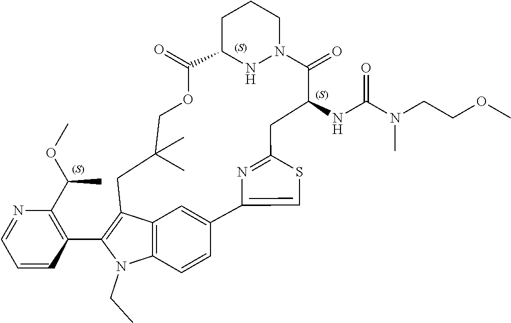 |
| DA 369 | 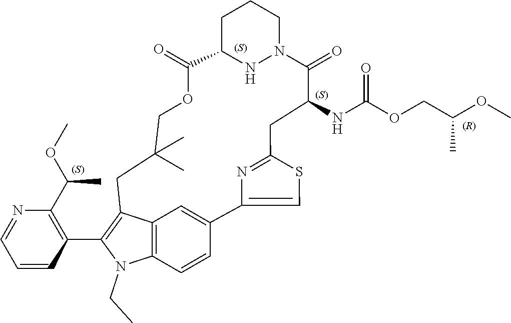 |
| DA 370 | 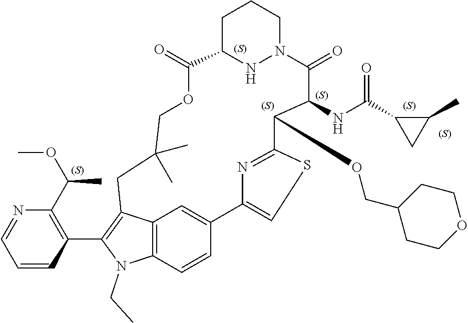 |
| DA 371 | 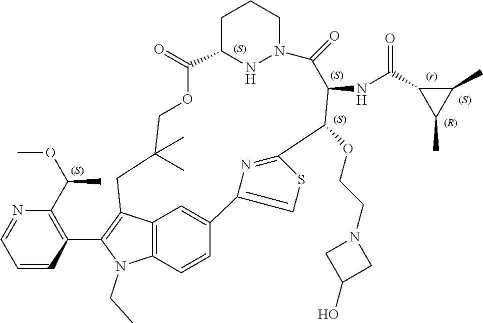 |
| DA 372 | 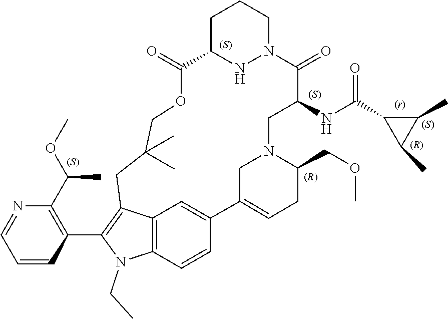 |
| DA 373 | 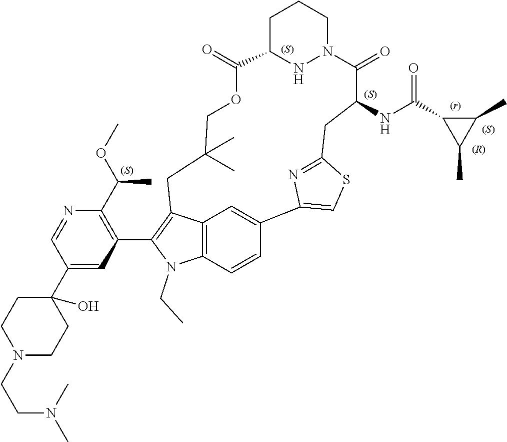 |
| DA 374 | 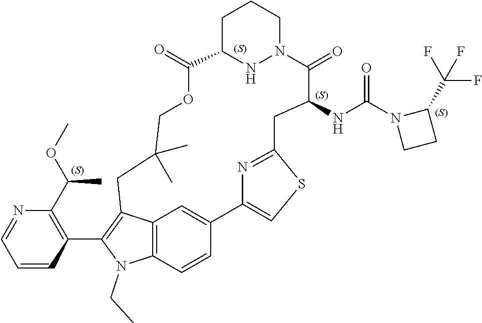 |
| DA 375 | 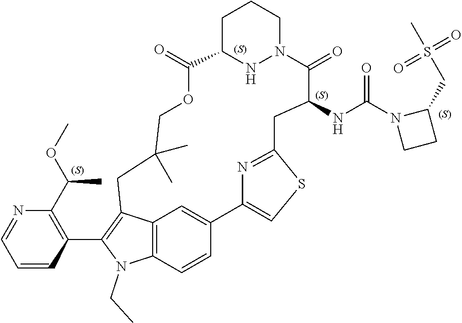 |
| DA 376 | 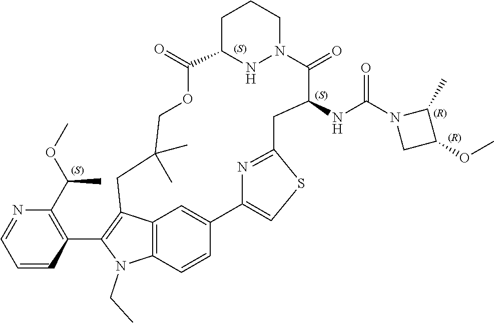 |
| DA 377 | 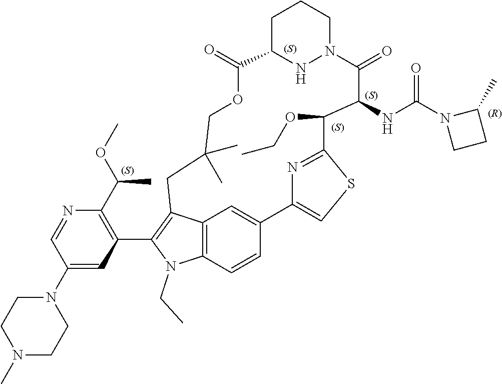 |
| DA 378 | 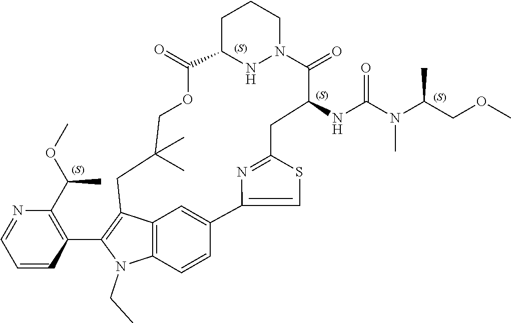 |
| DA 379 | 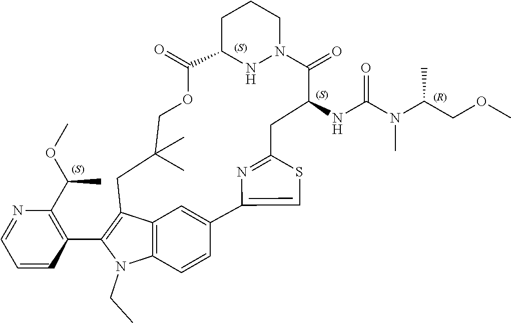 |
| DA 380 | 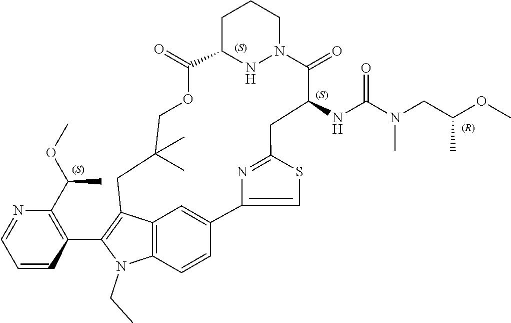 |
| DA 381 | 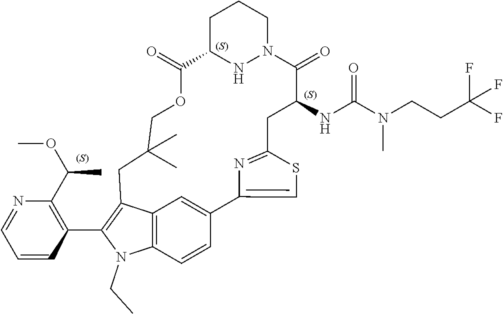 |
| DA 382 | 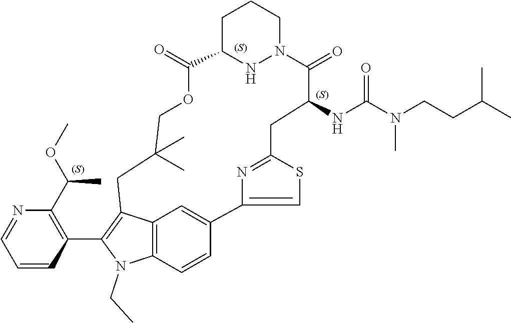 |
| DA 383 | 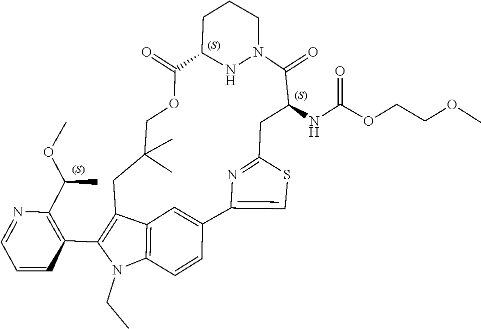 |
| DA 384 | 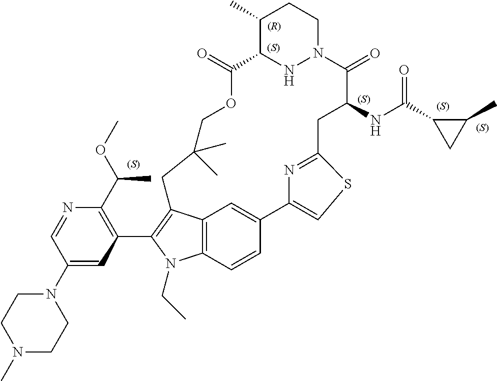 |
| DA 385 | 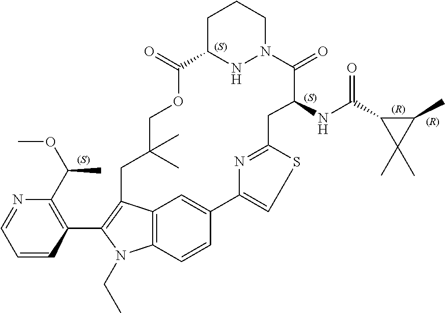 |
| DA 386 | 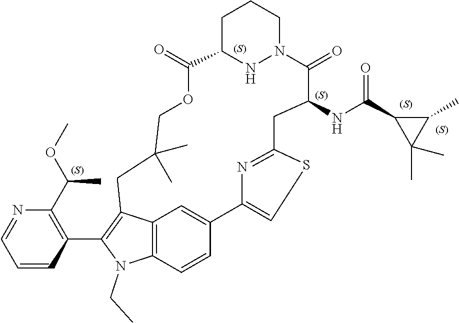 |
| DA 387 | 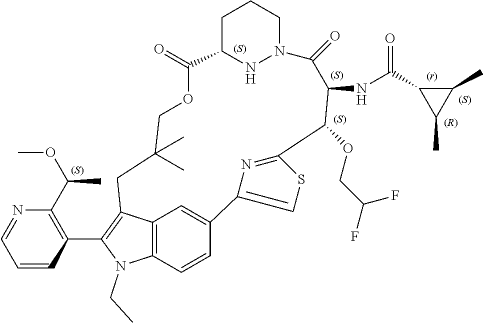 |
| DA 388 | 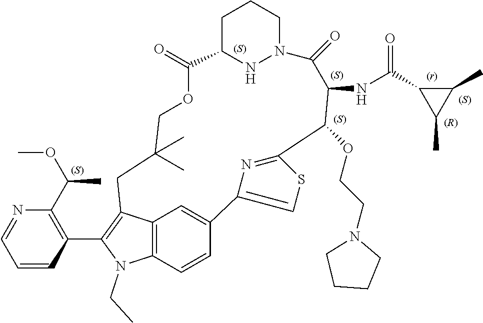 |
| DA 389 | 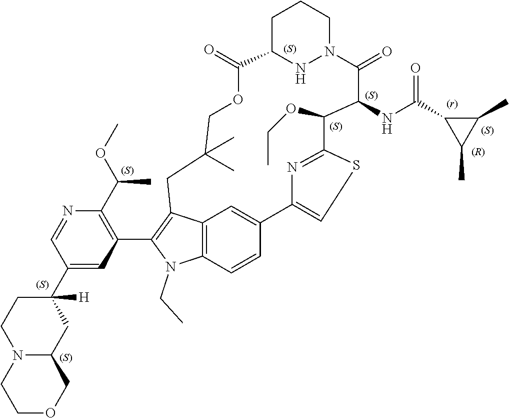 |
| DA 390 | 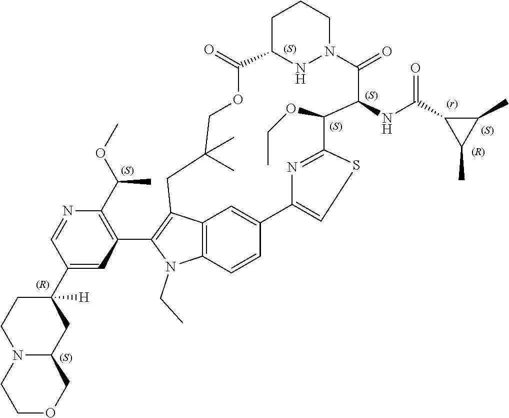 |
| DA 391 | 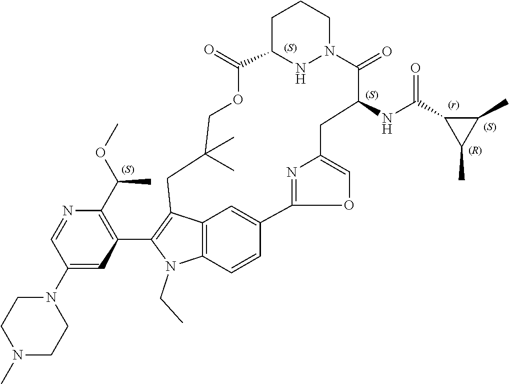 |
| DA 392 | 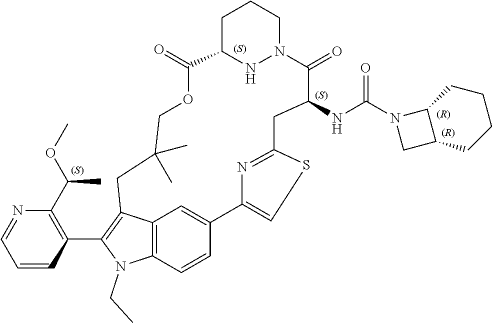 |
| DA 393 | 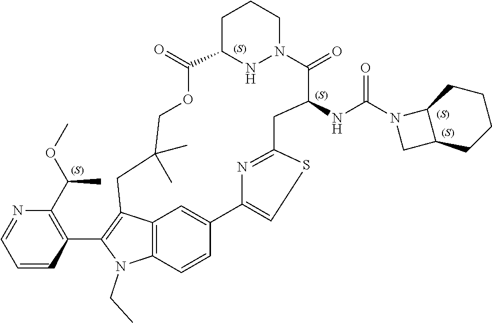 |
| DA 394 | 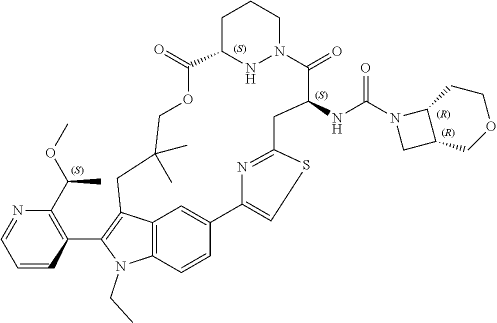 |
| DA 395 | 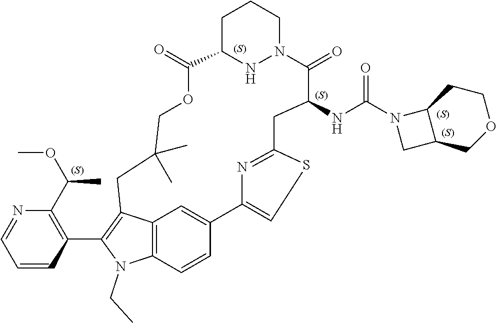 |
| DA 396 | 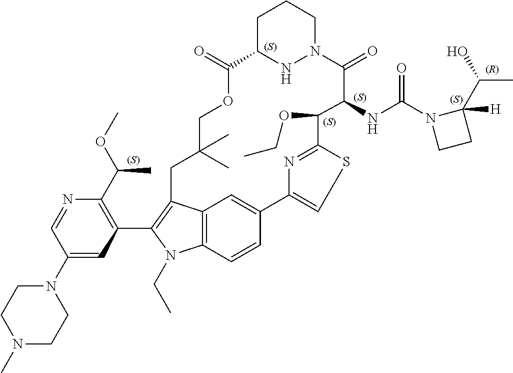 |
| DA 397 | 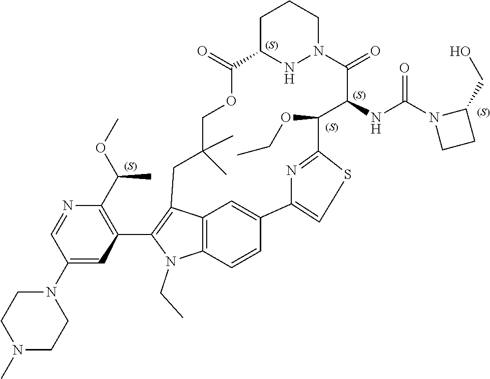 |
| DA 398 | 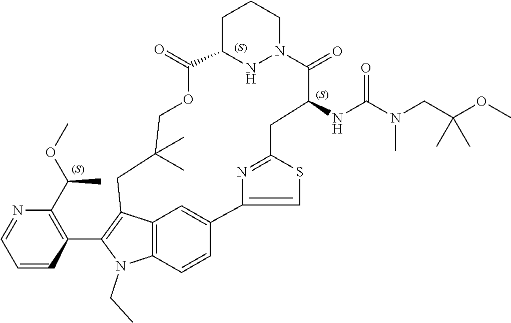 |
| DA 399 | 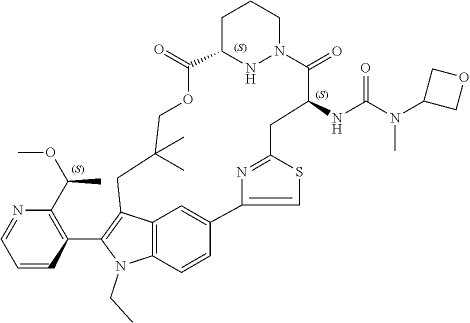 |
| DA 400 | 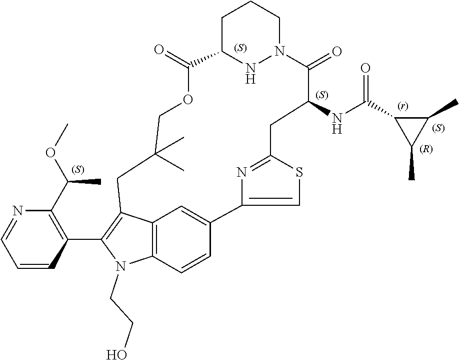 |
| DA 401 | 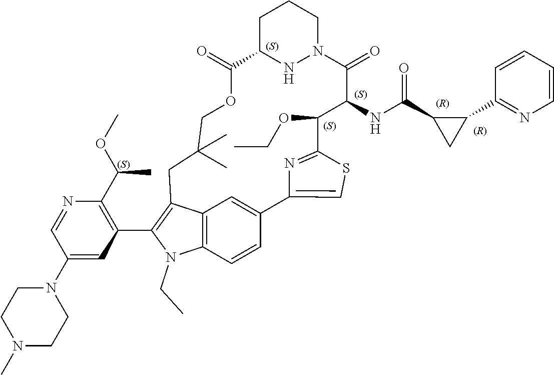 |
| DA 402 | 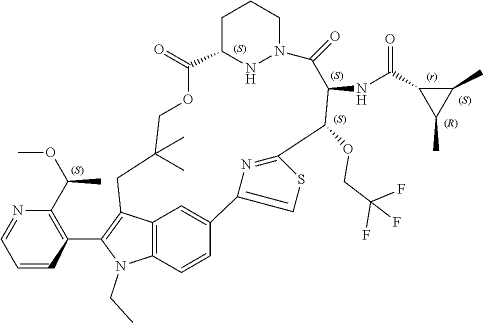 |
| DA 403 | 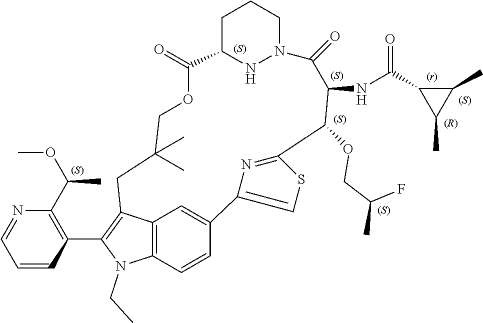 |
| DA 404 | 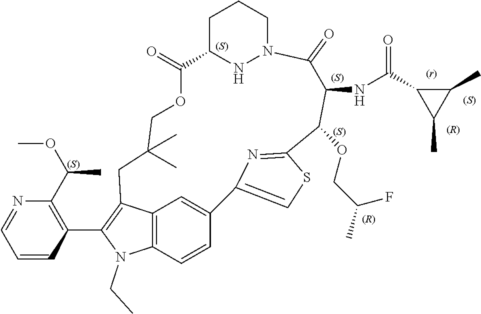 |
| DA 405 | 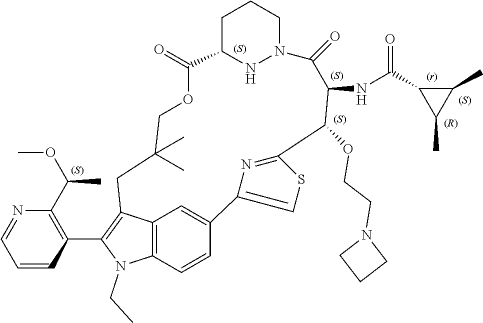 |
| DA 406 | 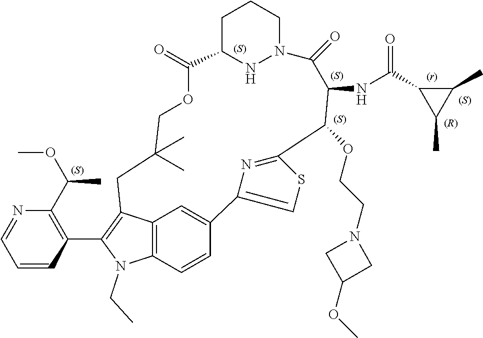 |
| DA 407 | 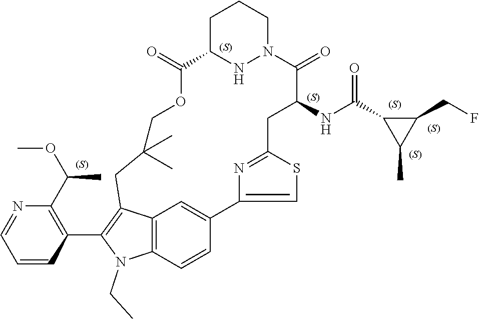 |
| DA 408 | 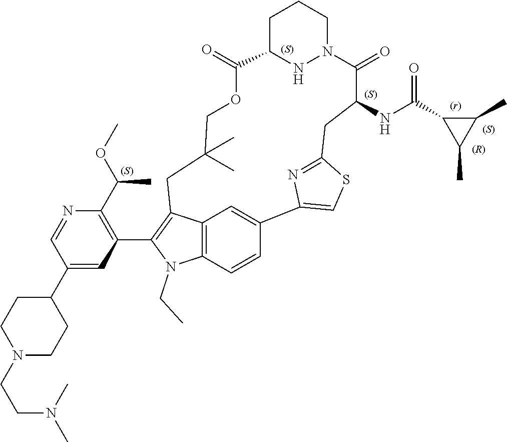 |
| DA 409 | 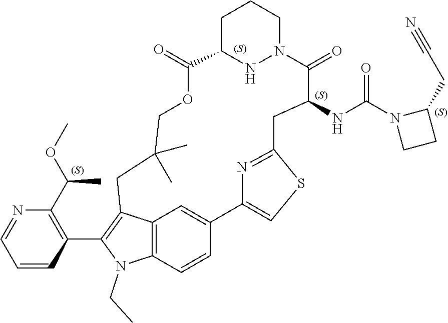 |
| DA 410 | 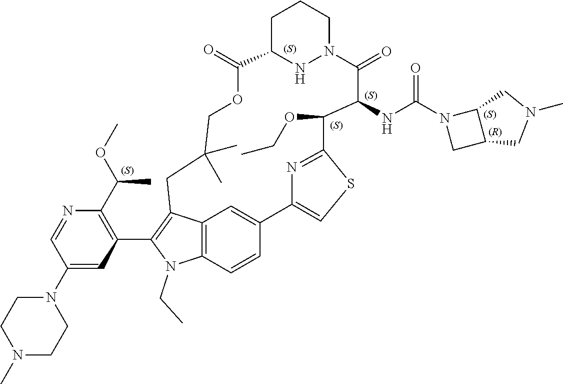 |
| DA 411 | 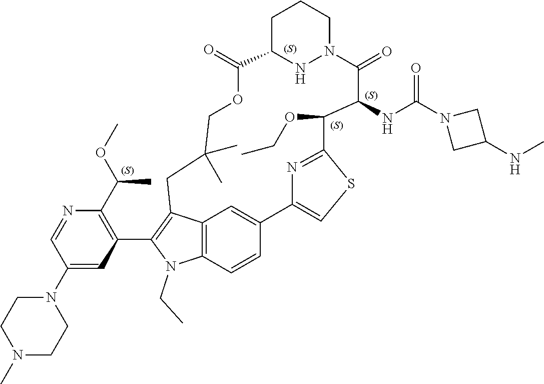 |
| DA 412 | 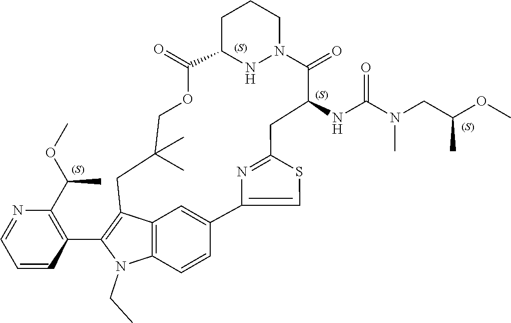 |
| DA 413 | 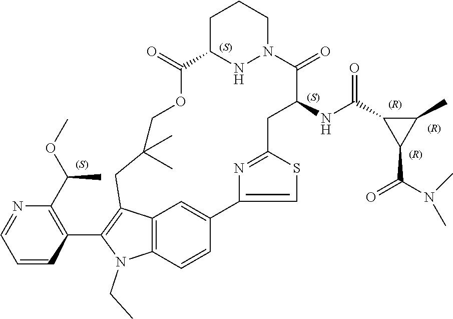 |
| DA 414 | 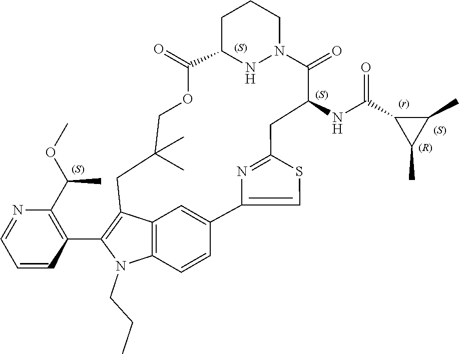 |
| DA 415 | 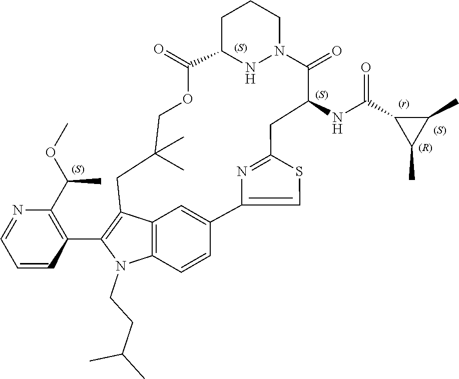 |
| DA 416 | 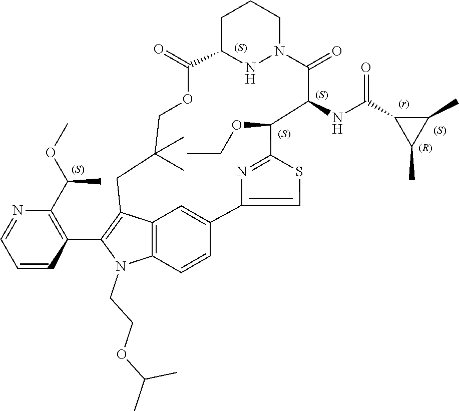 |
| DA 417 | 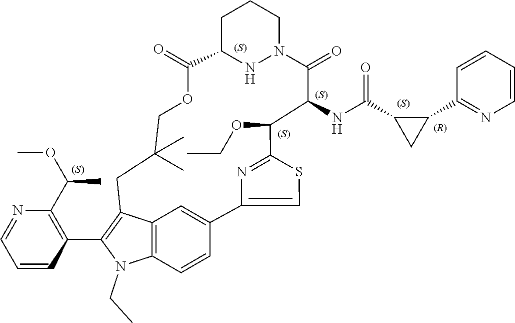 |
| DA 418 | 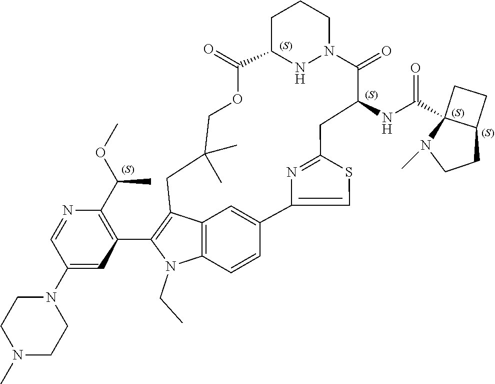 |
| DA 419 | 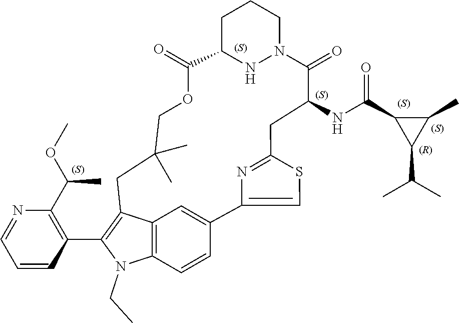 |
| DA 420 | 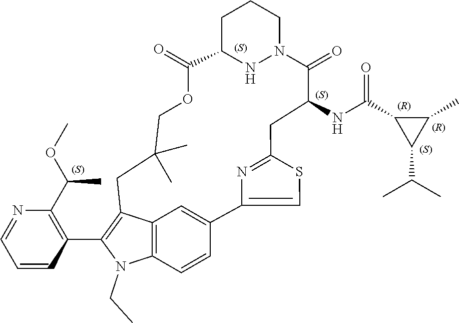 |
| DA 421 | 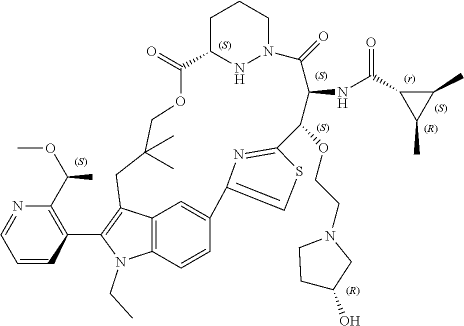 |
| DA 422 | 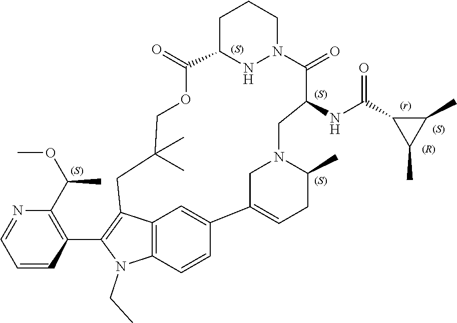 |
| DA 423 | 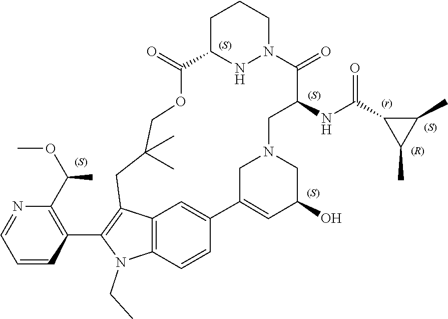 |
| DA 424 | 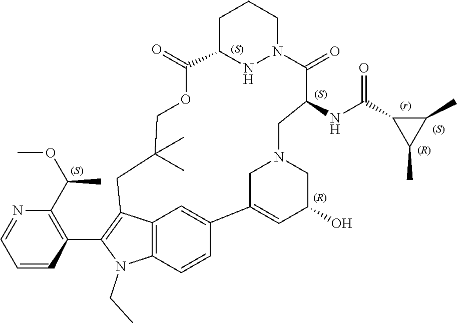 |
| DA 425 | 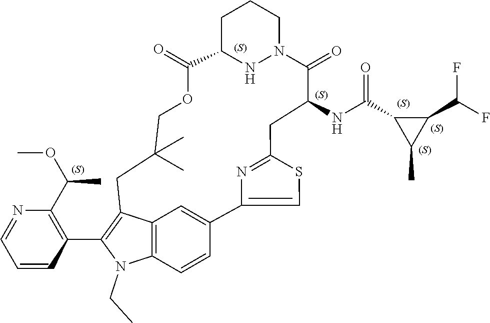 |
| DA 426 | 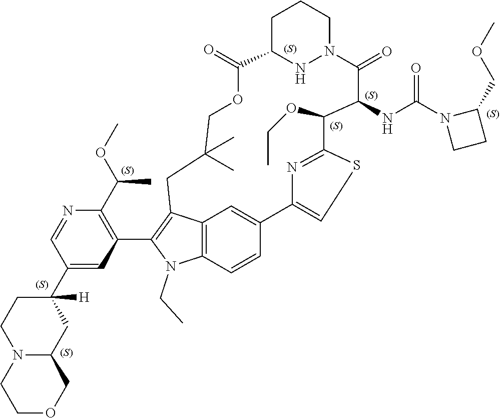 |
| DA 427 | 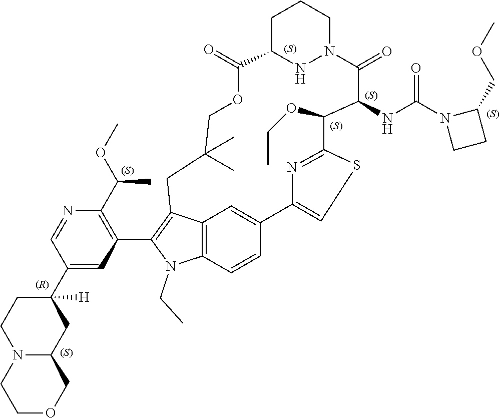 |
| DA 428 | 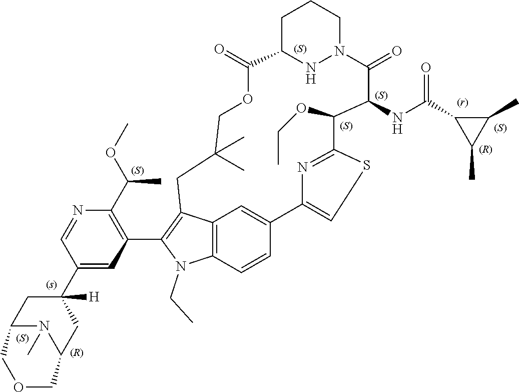 |
| DA 429 | 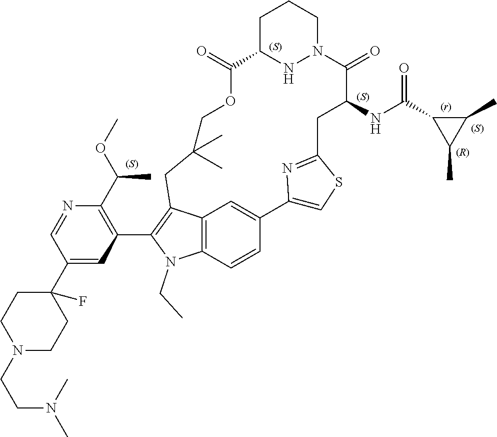 |
| DA 430 | 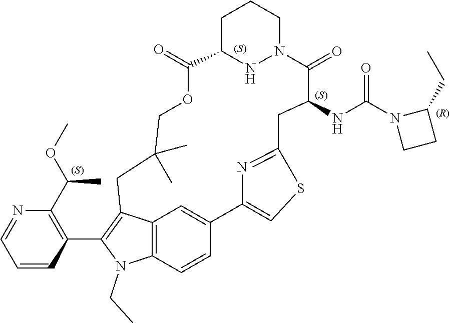 |
| DA 431 | 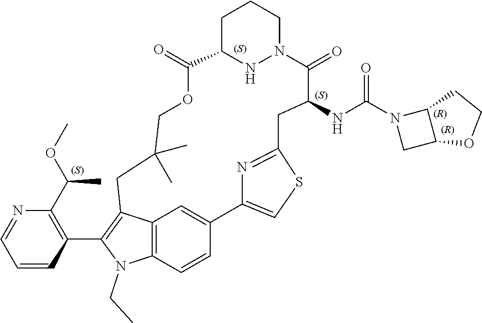 |
| DA 432 | 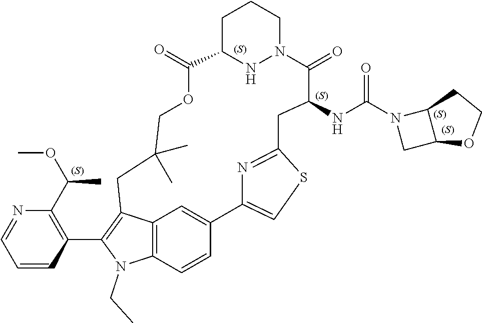 |
| DA 433 | 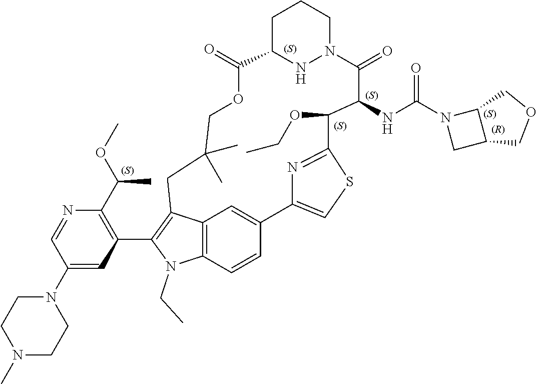 |
| DA 434 | 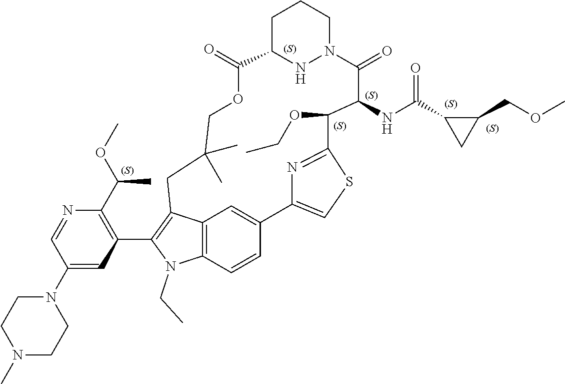 |
| DA 435 | 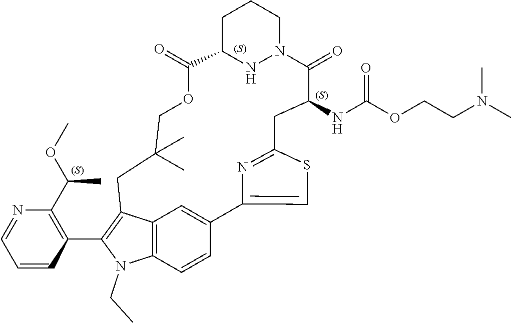 |
| DA 436 | 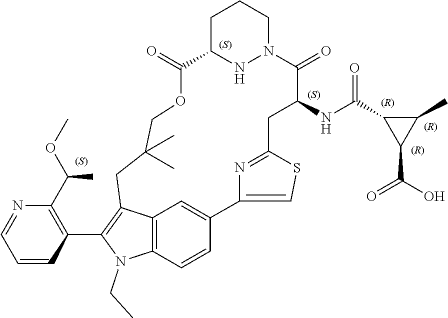 |
| DA 437 | 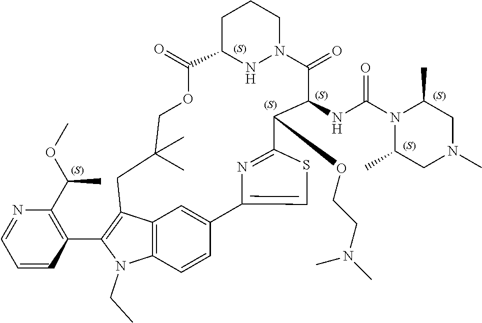 |
| DA 438 | 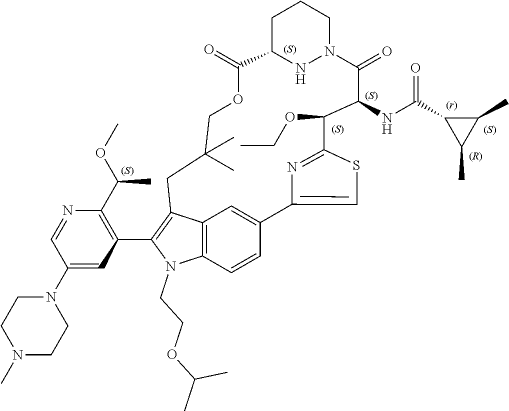 |
| DA 439 | 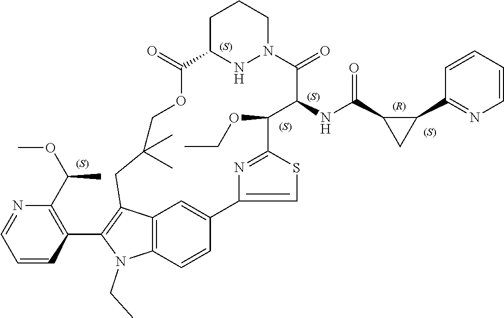 |
| DA 440 | 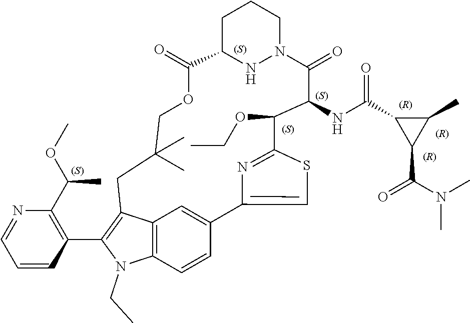 |
| DA 441 | 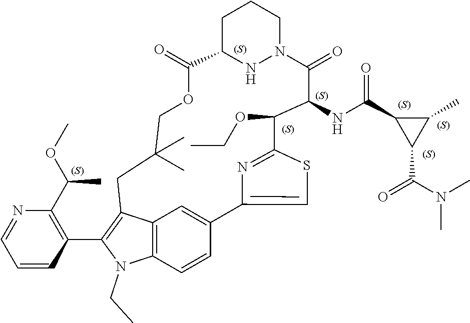 |
| DA 442 | 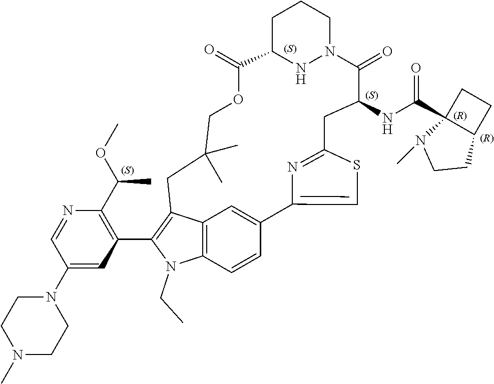 |
| DA 443 | 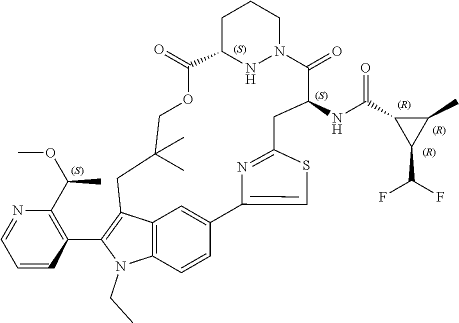 |
| DA 444 | 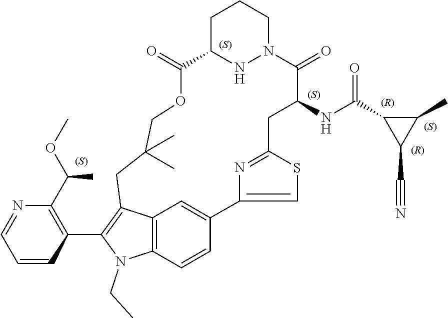 |
| DA 445 | 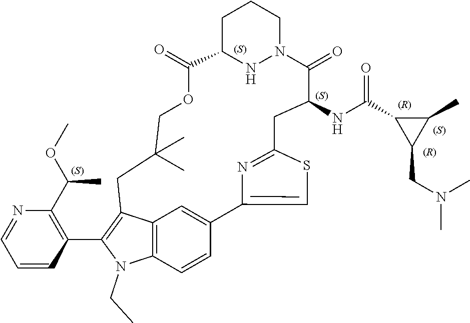 |
| DA 446 | 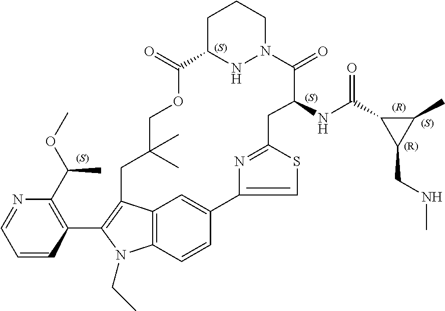 |
| DA 447 | 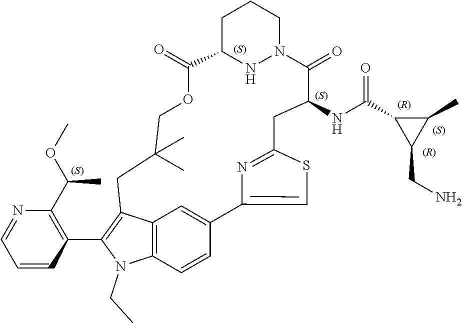 |
| DA 448 | 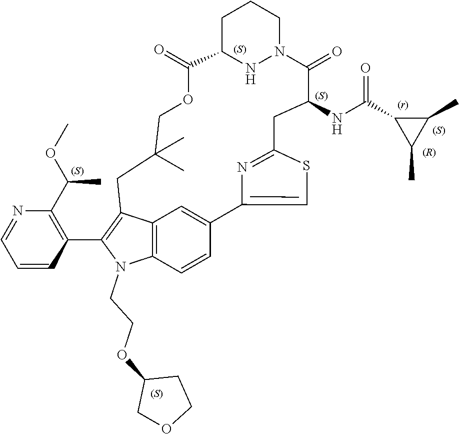 |
| DA 449 | 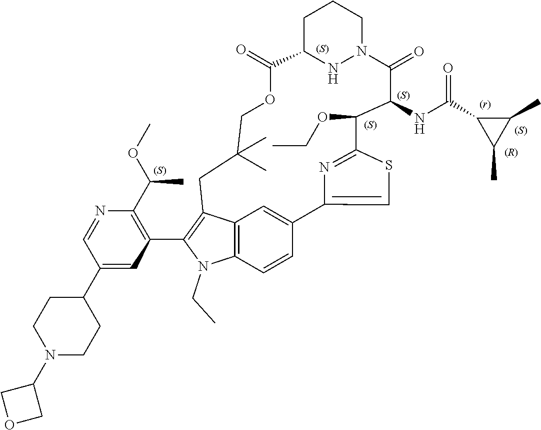 |
| DA 450 | 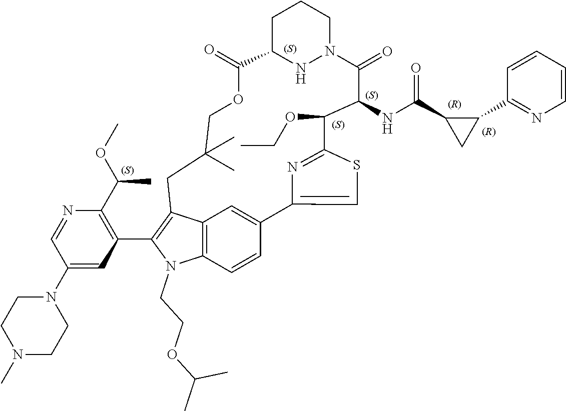 |
| DA 451 | 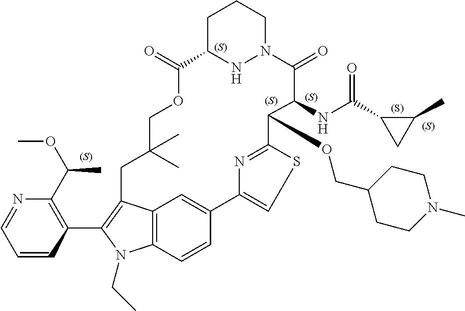 |
| DA 452 | 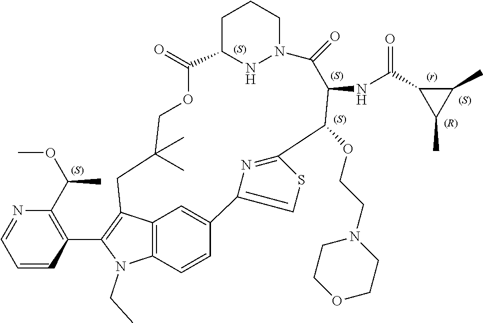 |
| DA 453 | 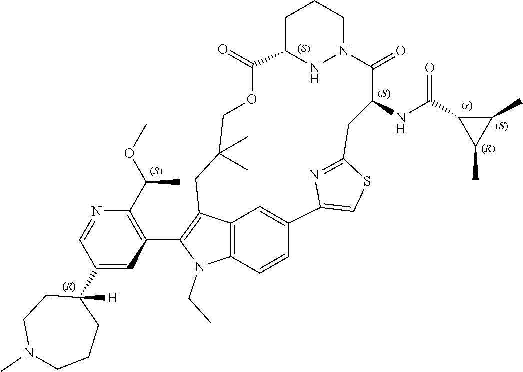 |
| DA 454 | 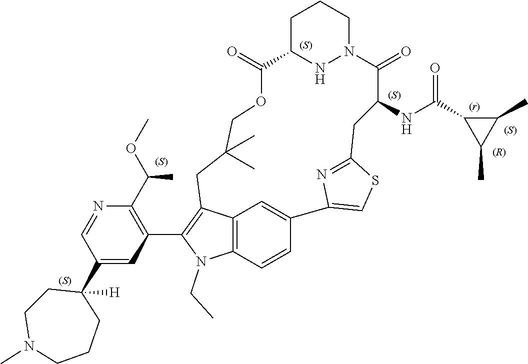 |
| DA 455 | 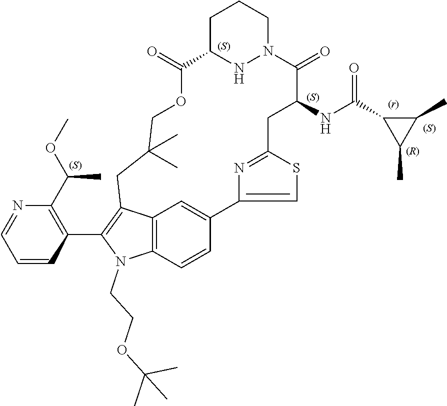 |
| DA 456 | 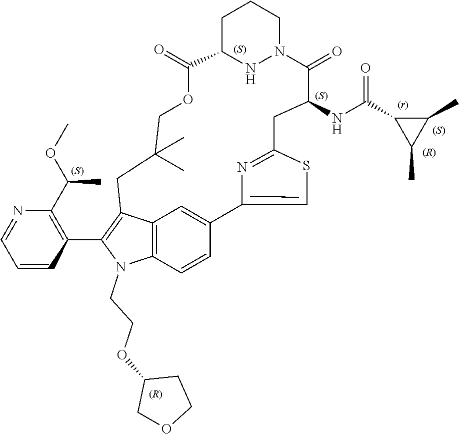 |
| DA 457 | 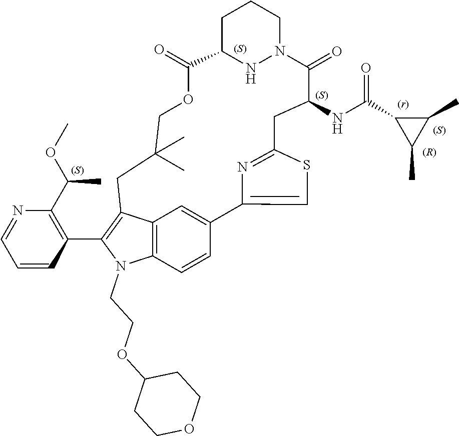 |
| DA 458 | 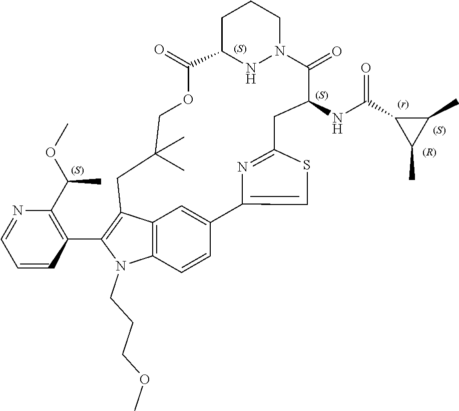 |
| DA 459 | 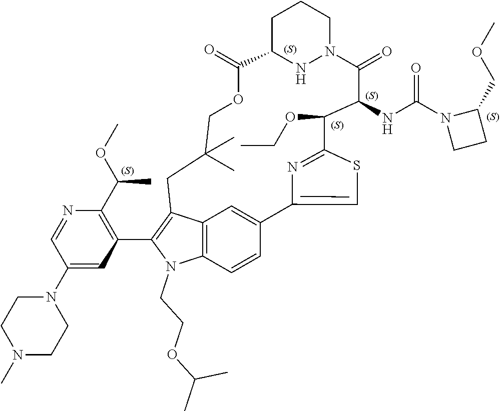 |
| DA 460 | 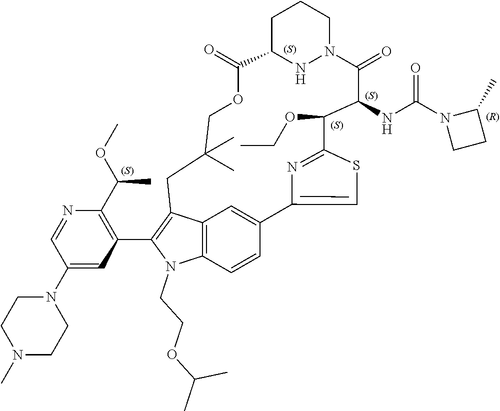 |
| DA 461 | 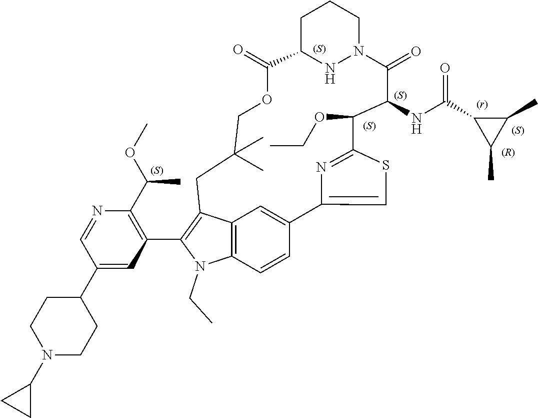 |
| DA 462 | 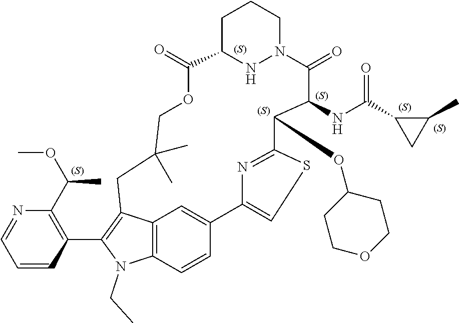 |
| DA 463 | 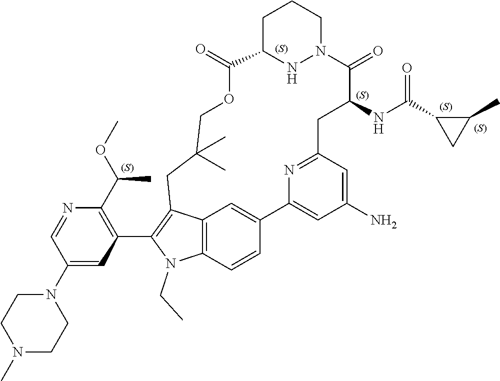 |
| DA 464 | 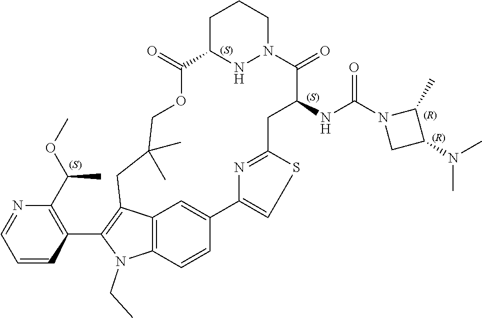 |
| DA 465 | 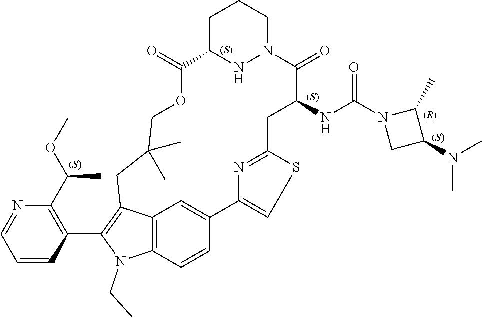 |
| DA 466 | 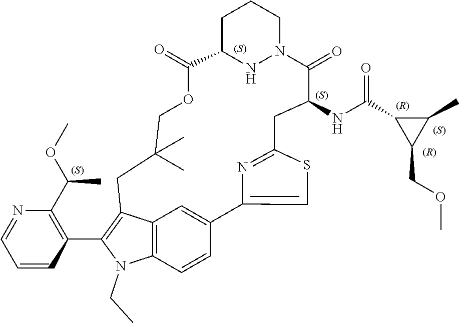 |
| DA 467 | 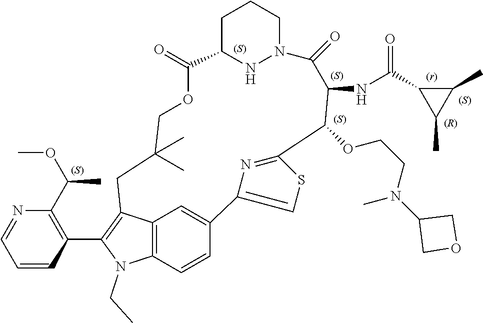 |
| DA 468 | 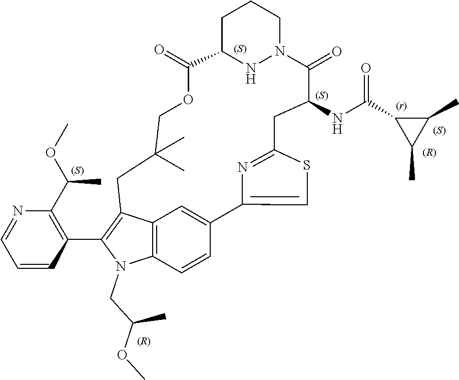 |
| DA 469 | 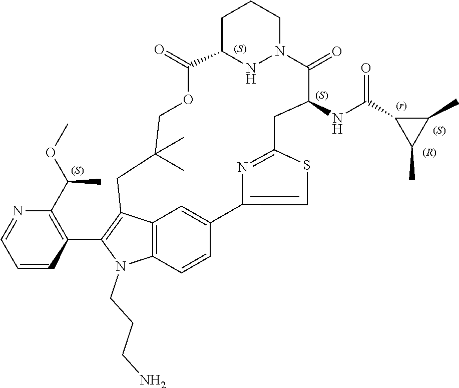 |
| DA 470 | 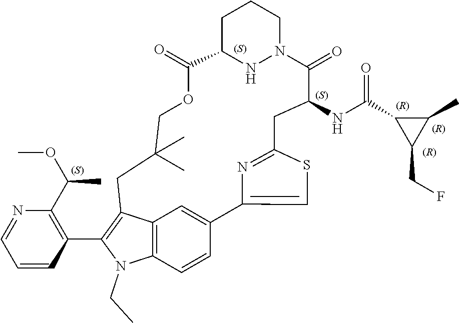 |
| DA 471 | 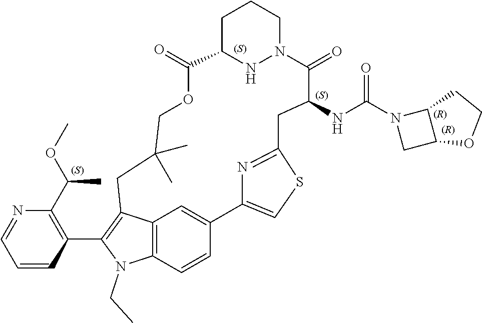 |
| DA 472 | 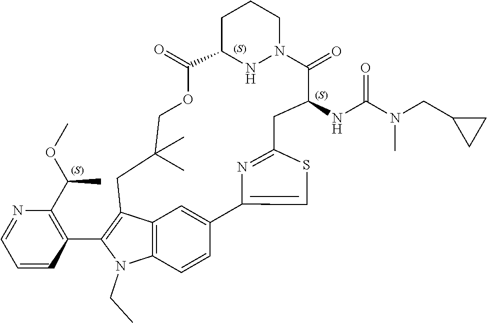 |
| DA 473 | 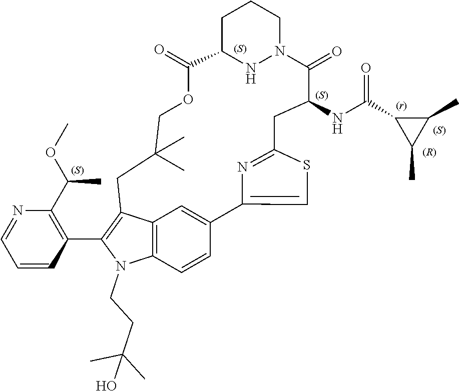 |
| DA 474 | 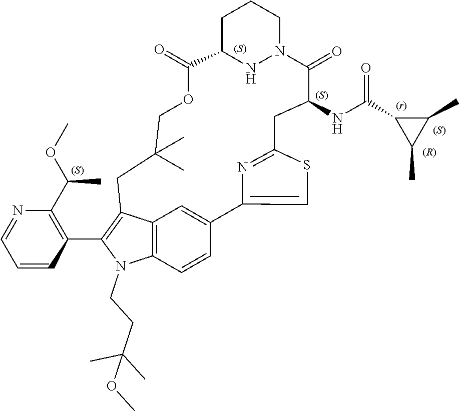 |
| DA 475 | 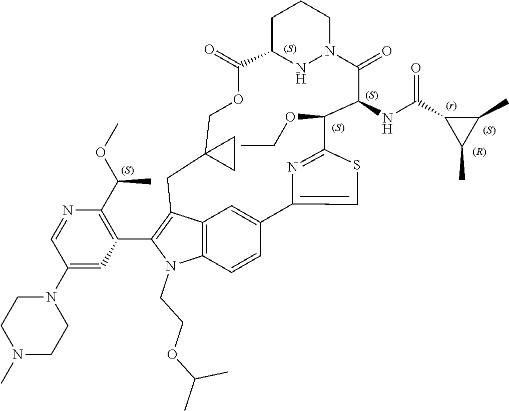 |
| DA 476 | 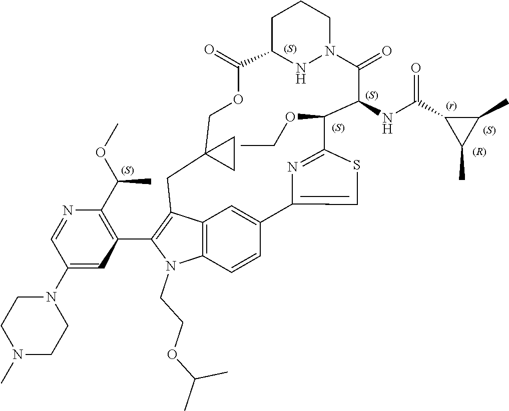 |
| DA 477 | 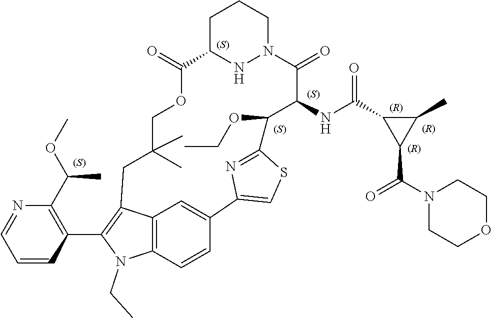 |
| DA 478 | 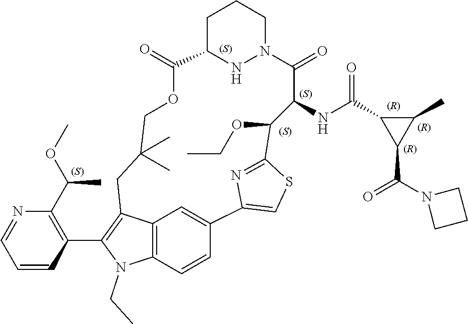 |
| DA 479 | 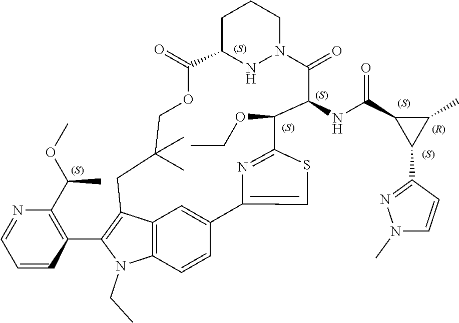 |
| DA 480 | 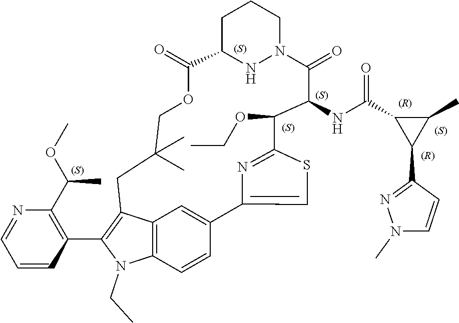 |
| DA 481 | 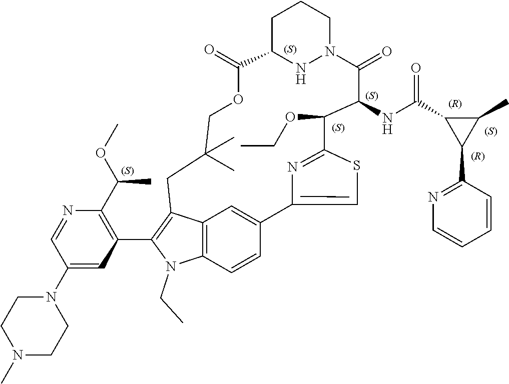 |
| DA 482 | 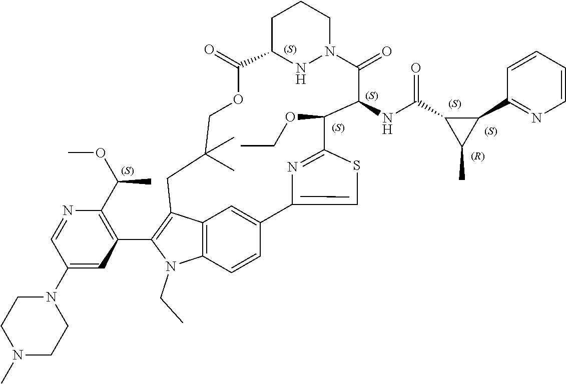 |
| DA 483 | 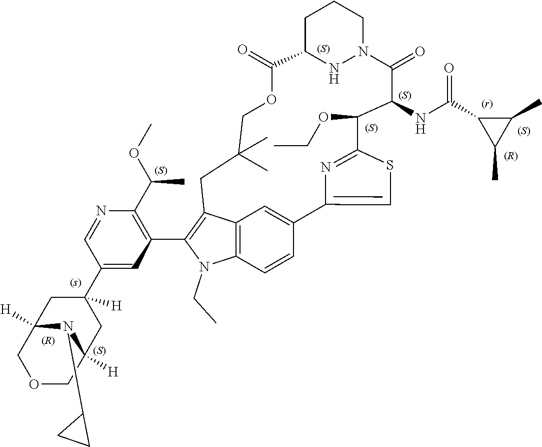 |
| DA 484 | 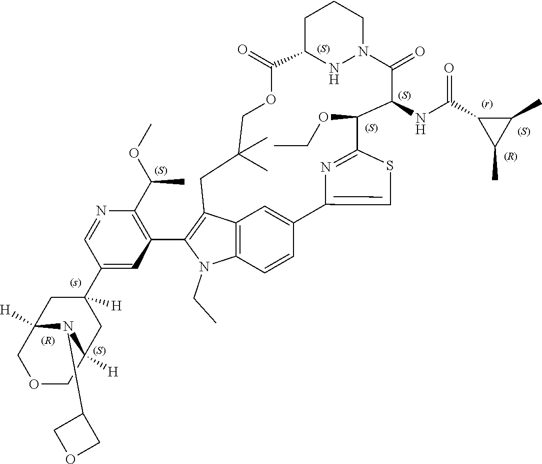 |
| DA 485 | 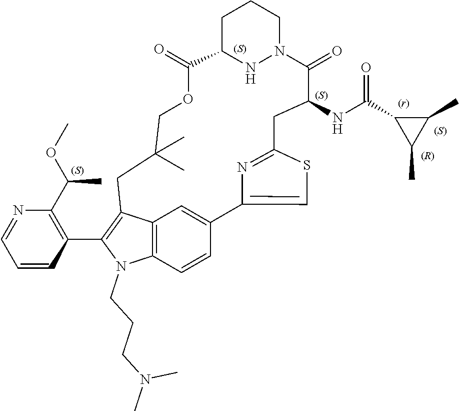 |
| DA 486 | 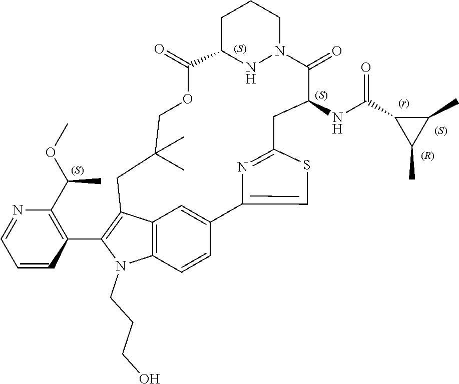 |
| DA 487 | 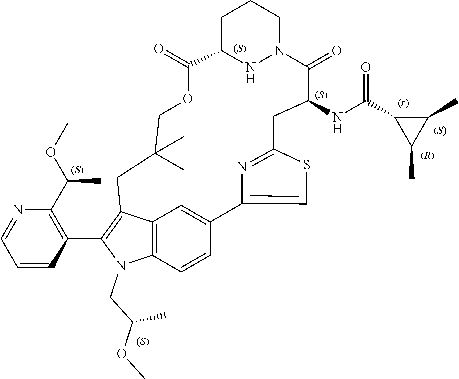 |
| DA 488 | 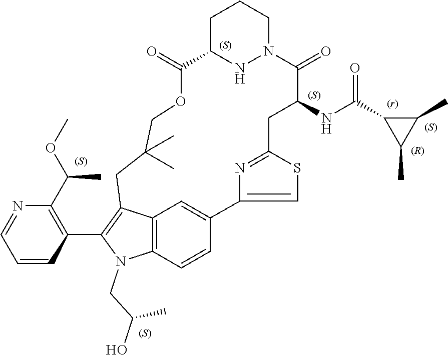 |
| DA 489 | 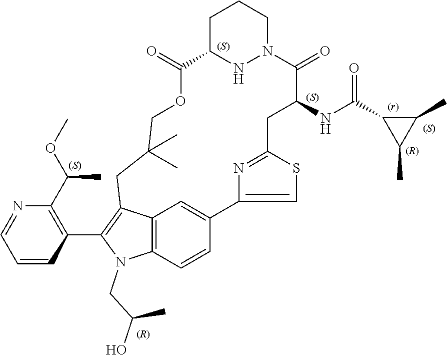 |
| DA 490 | 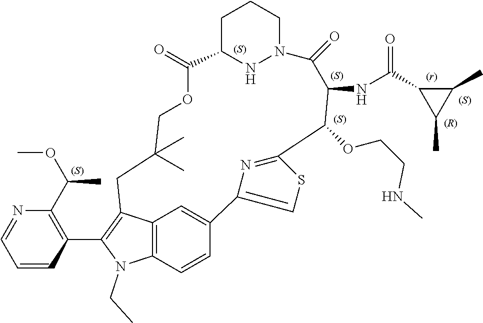 |
| DA 491 | 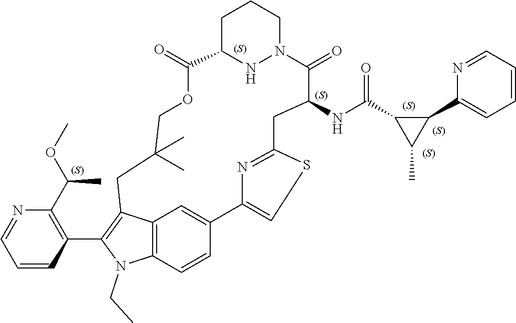 |
| DA 492 | 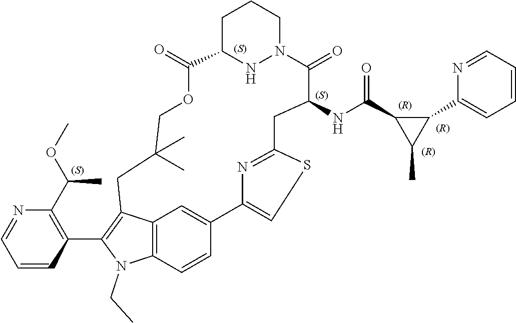 |
| DA 493 | 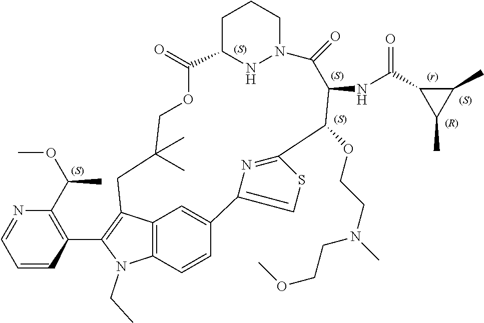 |
| DA 494 | 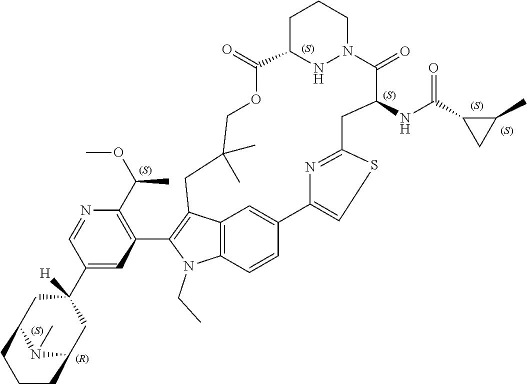 |
| DA 495 | 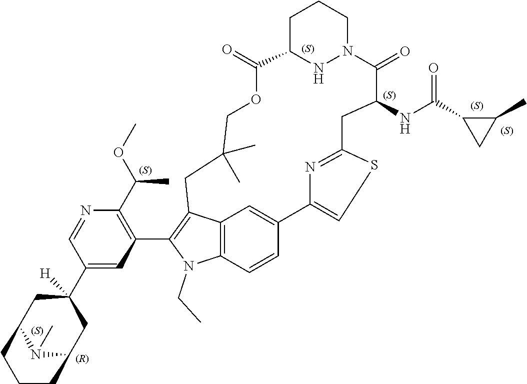 |
| DA 496 | 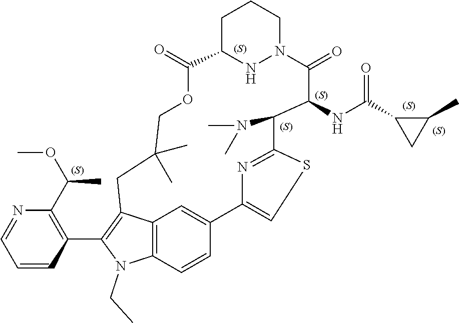 |
| DA 497 | 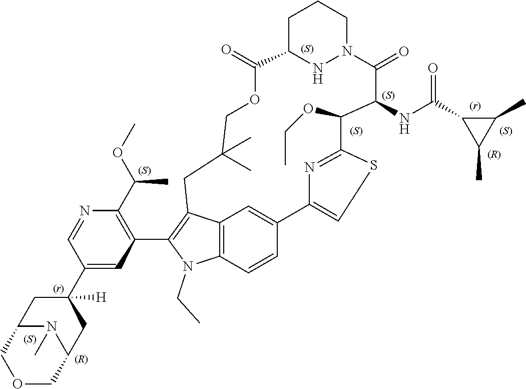 |
| DA 498 | 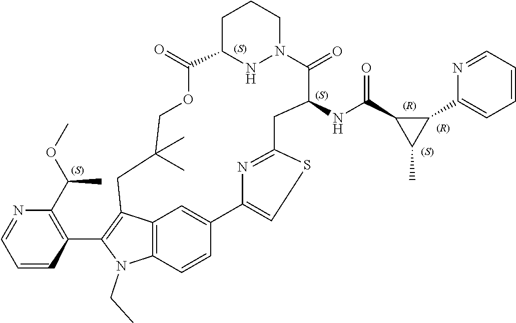 |
| DA 499 | 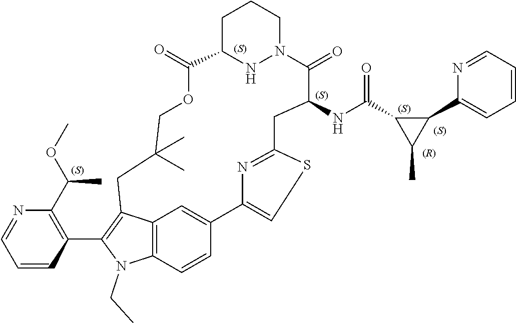 |
| DA 500 | 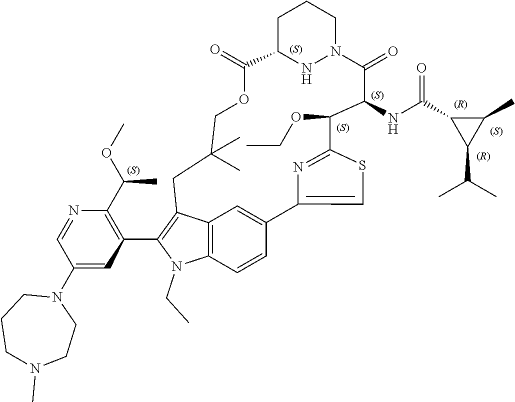 |
| DA 501 | 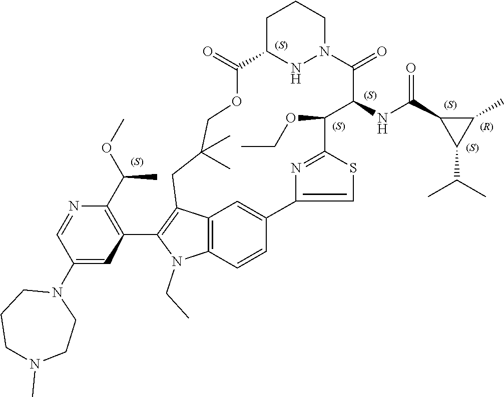 |
| DA 502 | 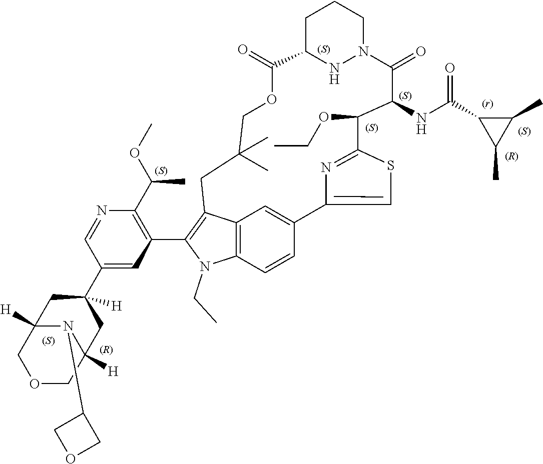 |
| DA 503 | 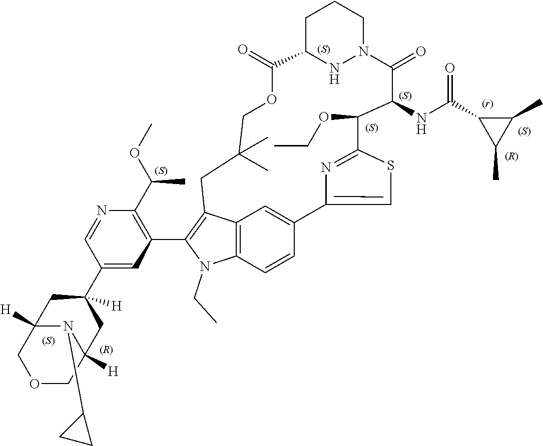 |
| DA 504 | 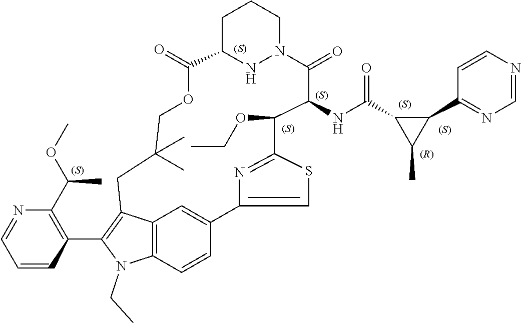 |
| DA 505 | 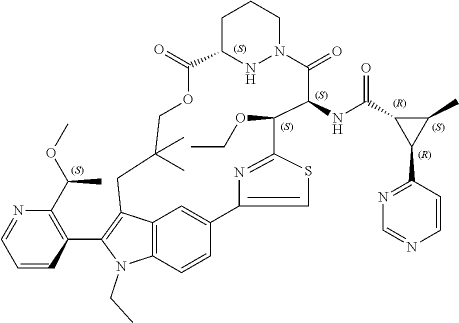 |
| DA 506 | 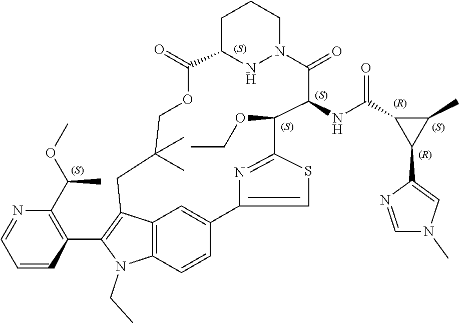 |
| DA 507 | 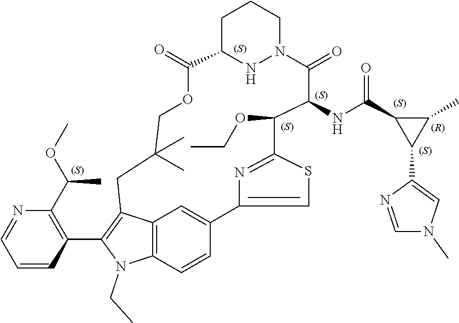 |
| DA 508 | 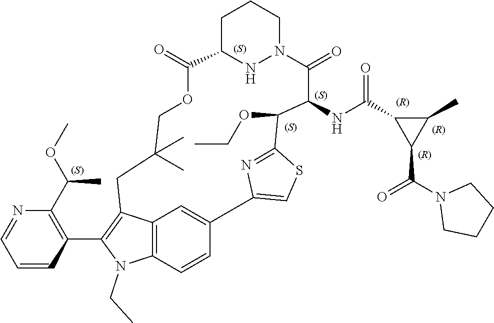 |
| DA 509 | 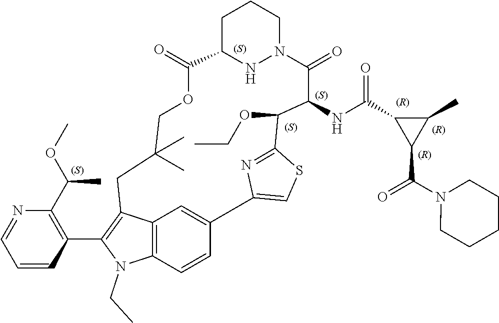 |
| DA 510 | 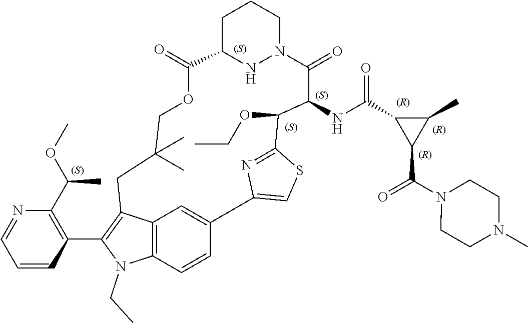 |
| DA 511 | 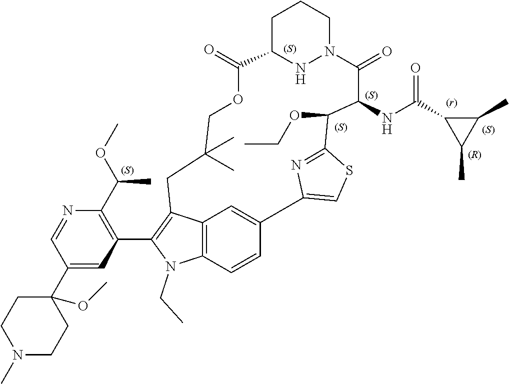 |
| DA 512 | 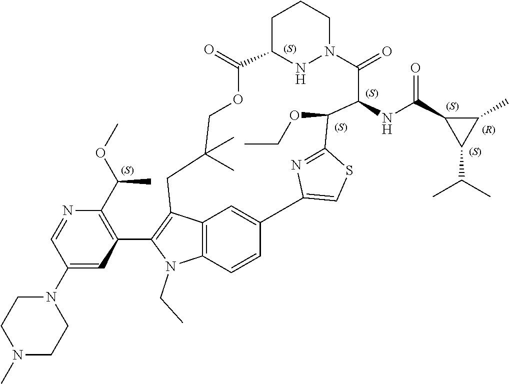 |
| DA 513 | 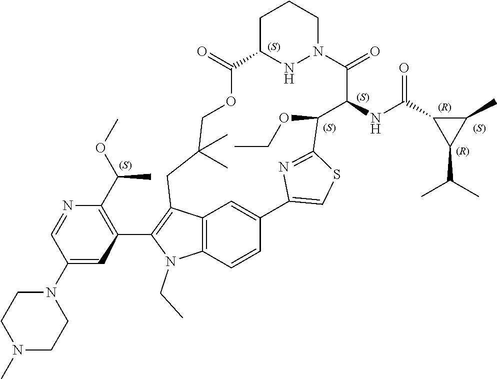 |
| DA 514 | 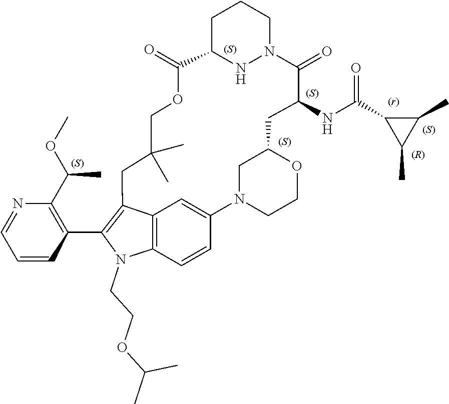 |
| DA 515 | 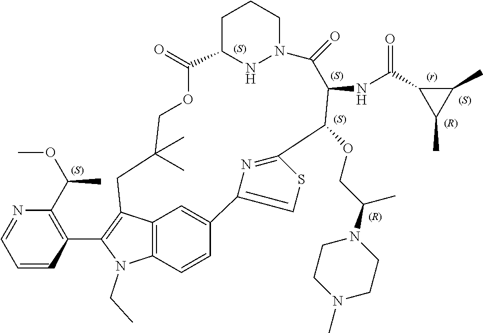 |
| DA 516 | 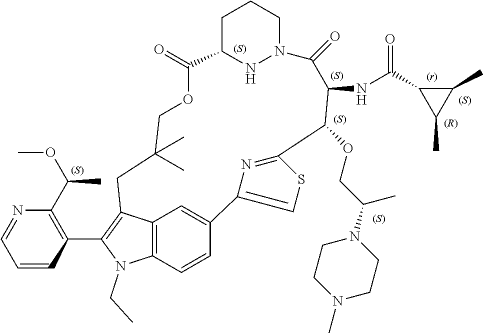 |
| DA 517 | 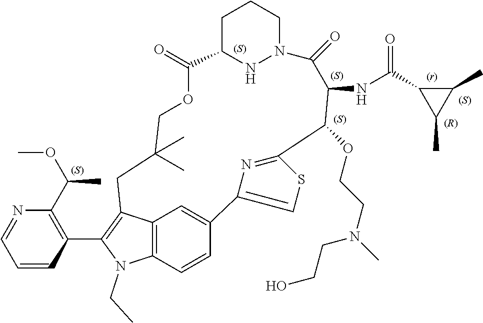 |
| DA 518 | 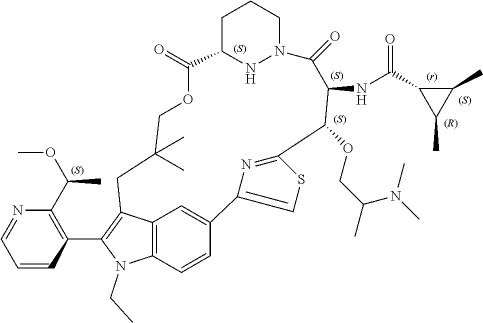 |
| DA 519 | 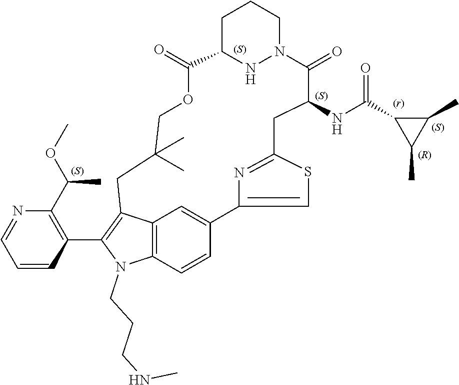 |
| DA 520 | 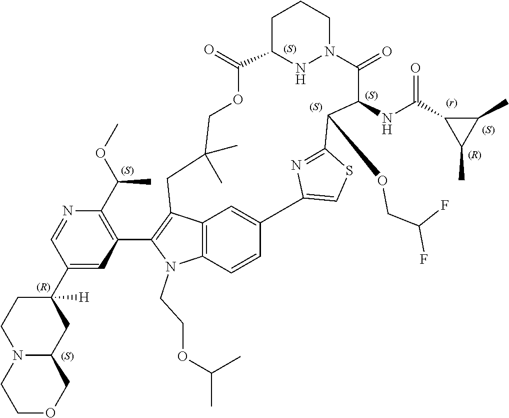 |
| DA 521 | 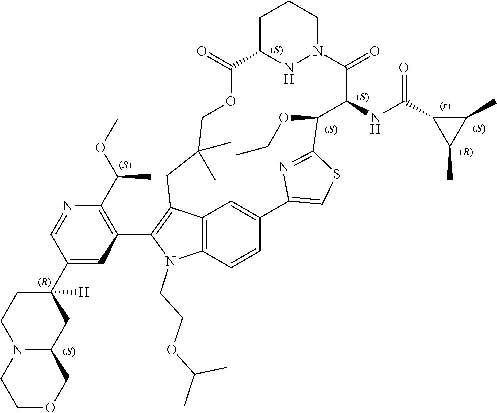 |
| DA 522 | 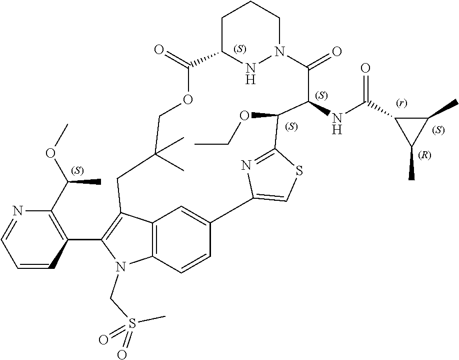 |
| DA 523 | 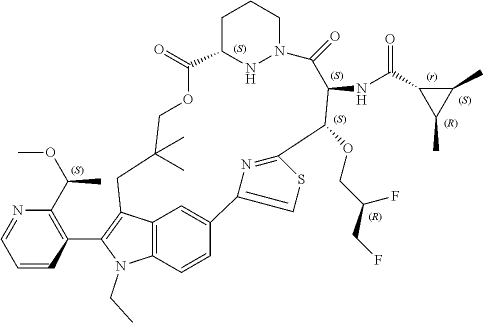 |
| DA 524 | 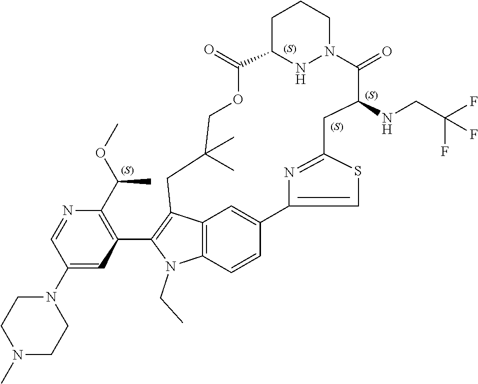 |
| DA 525 | 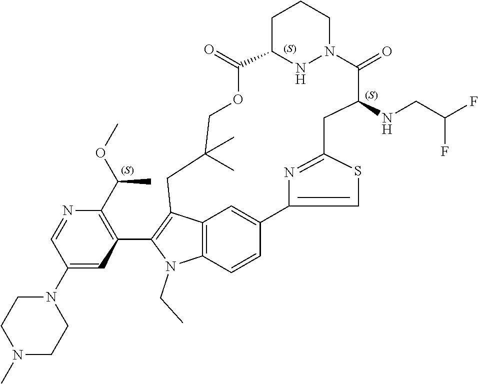 |
| DA 526 | 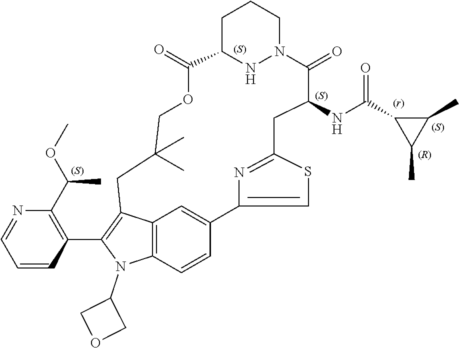 |
| DA 527 | 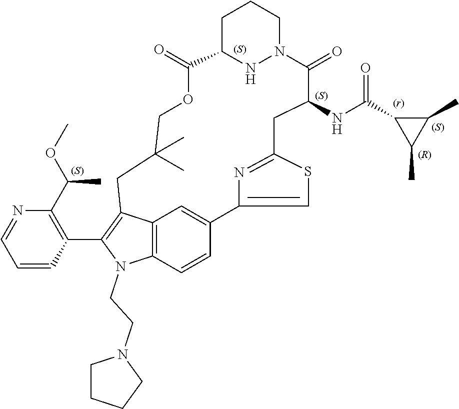 |
| DA 528 | 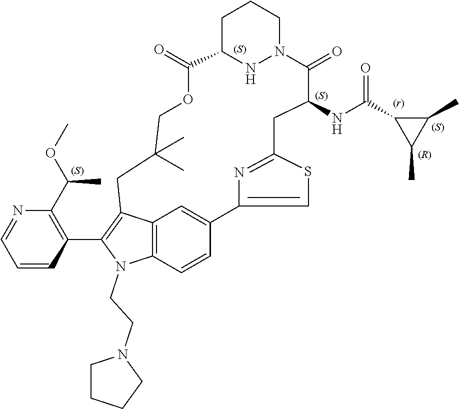 |
| DA 529 | 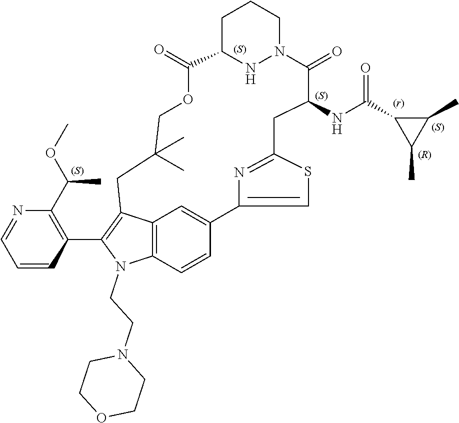 |
| DA 530 | 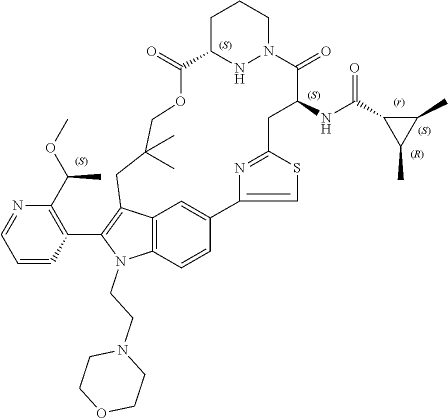 |
| DA 531 | 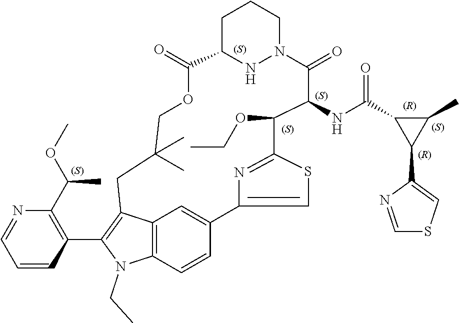 |
| DA 532 | 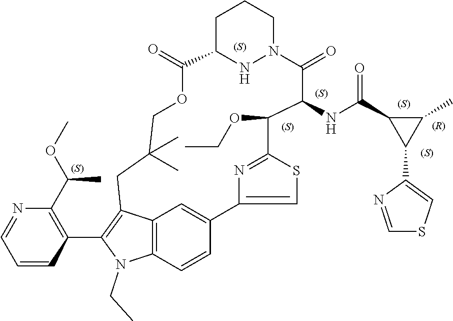 |
| DA 533 | 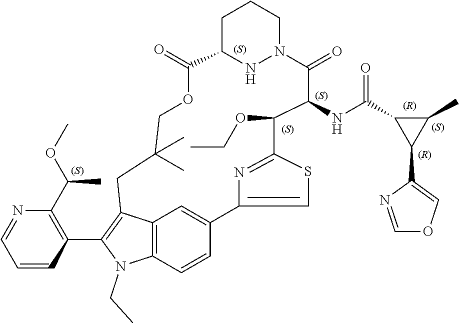 |
| DA 534 | 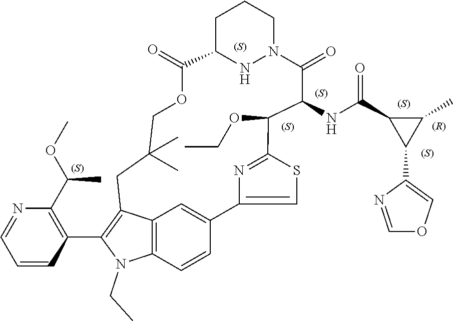 |
| DA 535 | 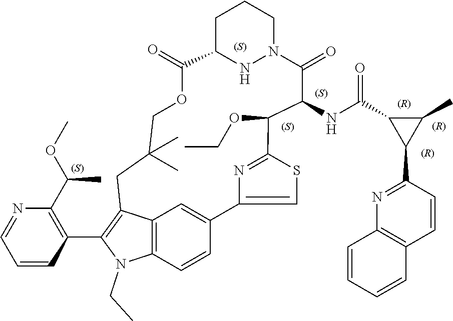 |
| DA 536 | 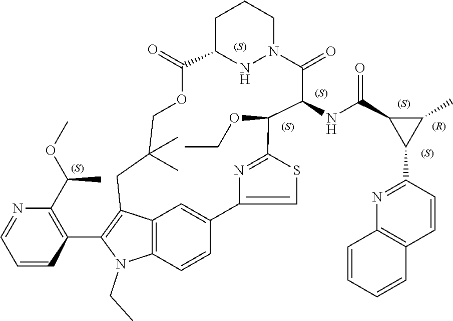 |
| DA 537 | 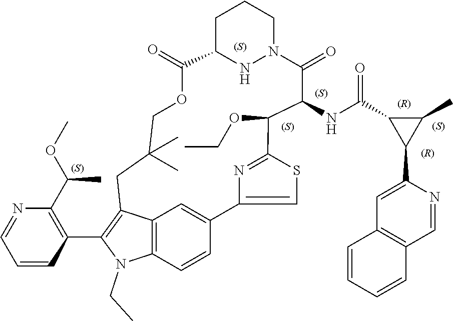 |
| DA 538 | 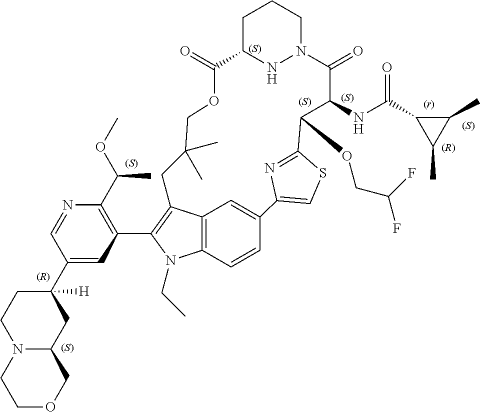 |
| DA 539 | 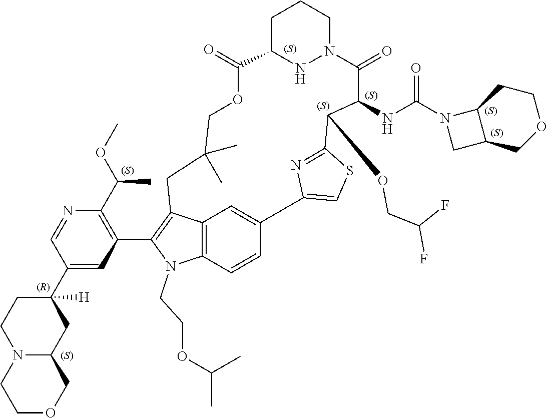 |
| DA 540 | 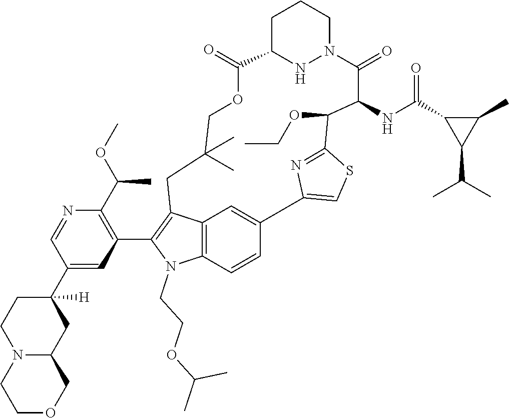 |
| DA 541 | 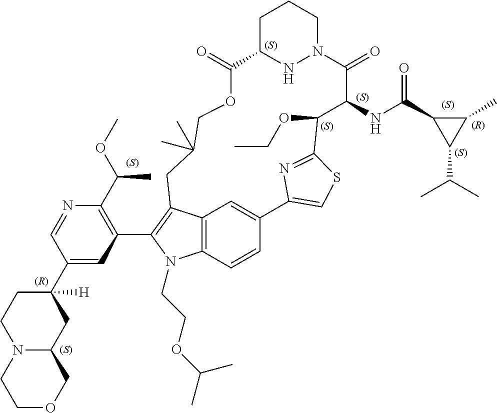 |
| DA 542 | 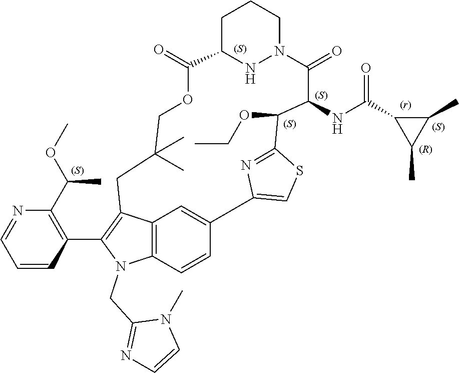 |
| DA 543 | 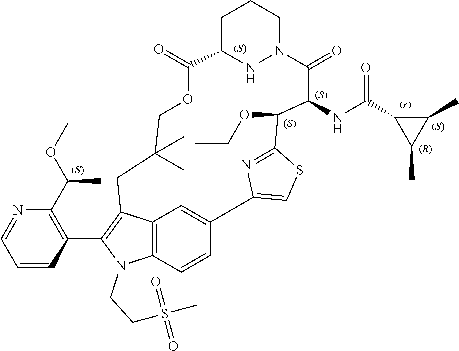 |
| DA 544 | 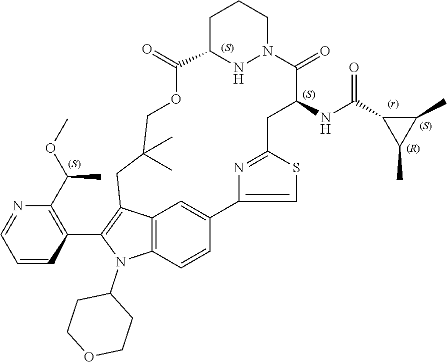 |
| DA 545 | 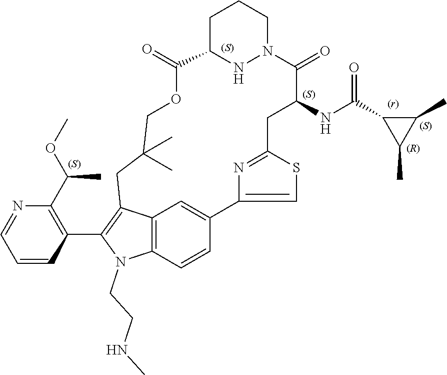 |
| DA 546 | 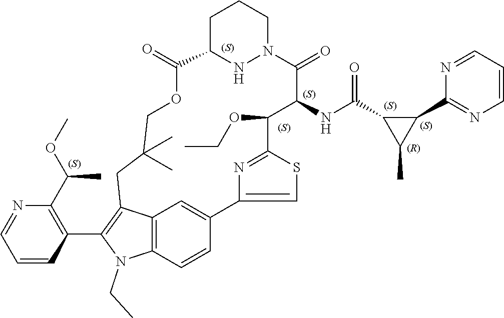 |
| DA 547 | 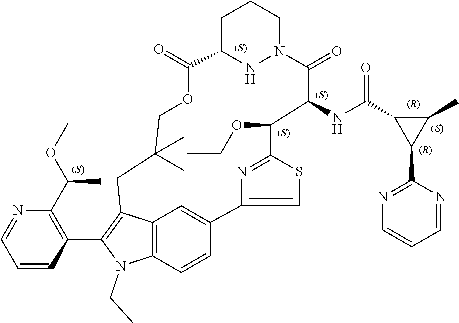 |
| DA 548 | 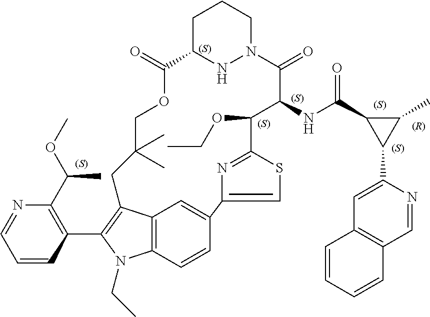 |
| DA 549 | 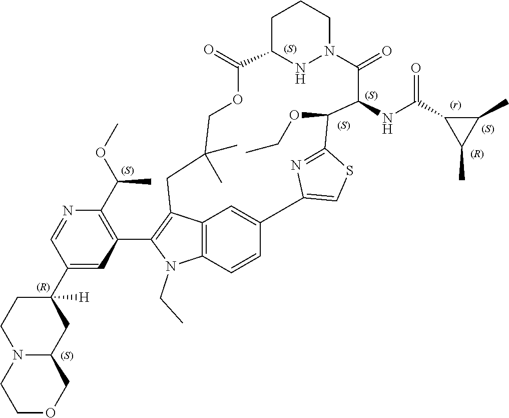 |
| DA 550 | 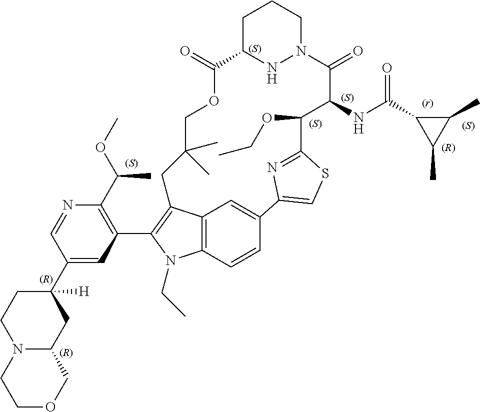 |
| DA 551 | 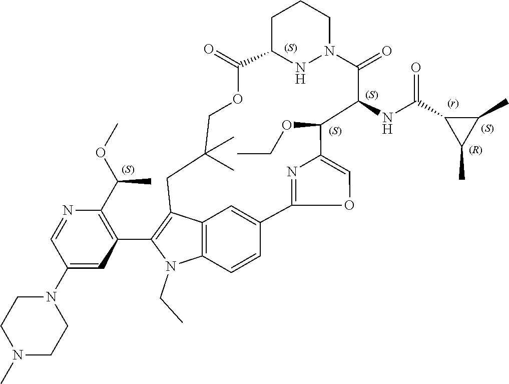 |
| DA 552 | 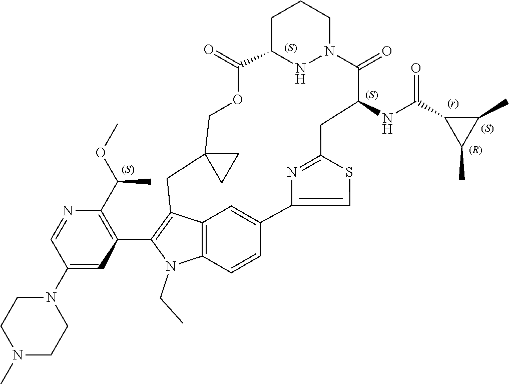 |
| DA 553 | 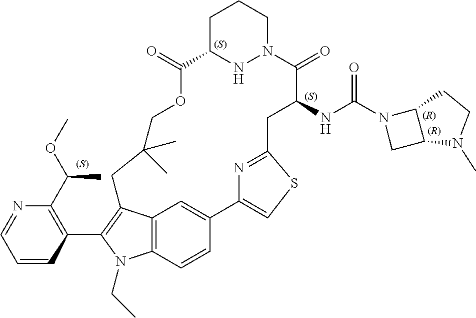 |
| DA 554 | 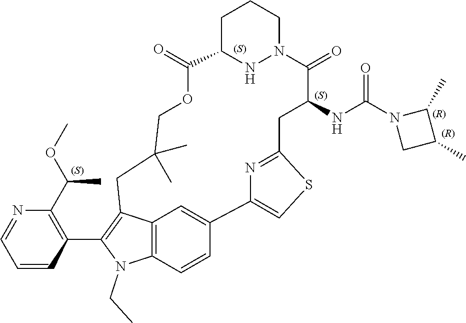 |
| DA 555 | 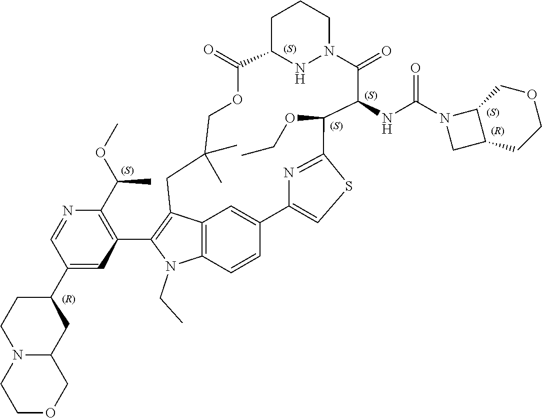 |
| DA 556 | 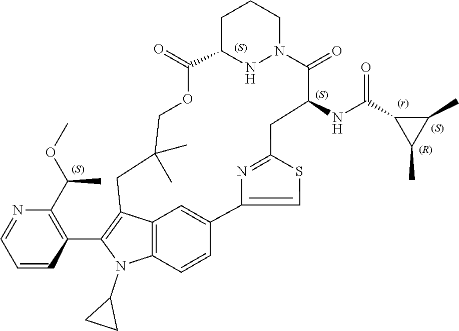 |
| DA 557 | 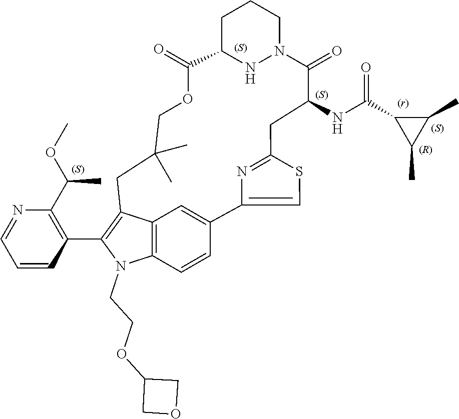 |
| DA 558 | 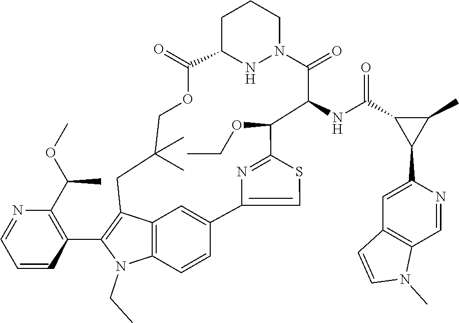 |
| DA 559 | 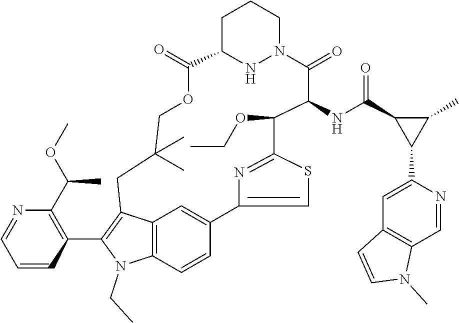 |
| DA 560 | 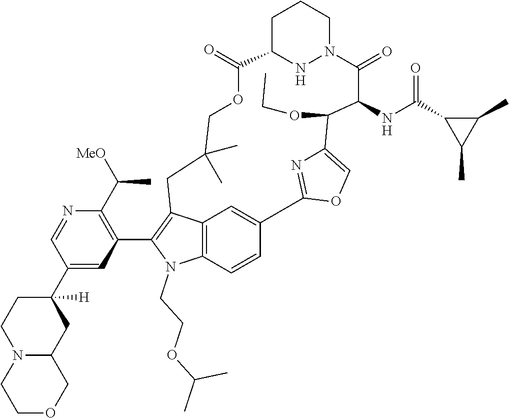 |
| DA 561 | 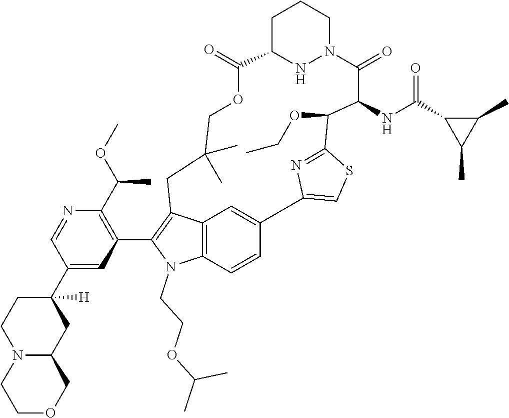 |
| DA 562 | 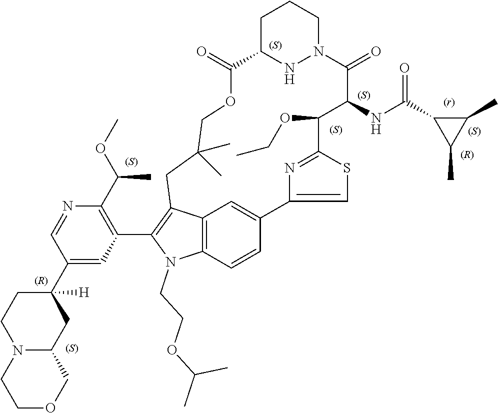 |
| DA 563 | 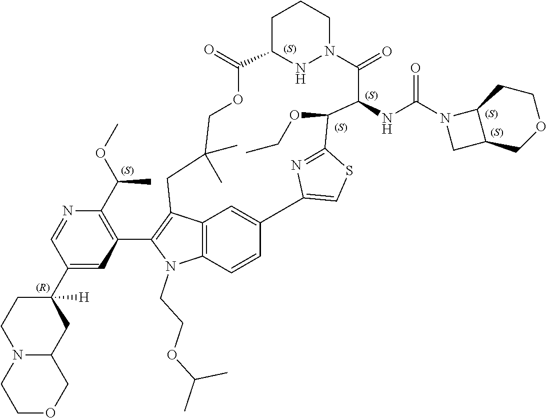 |
| DA 564 | 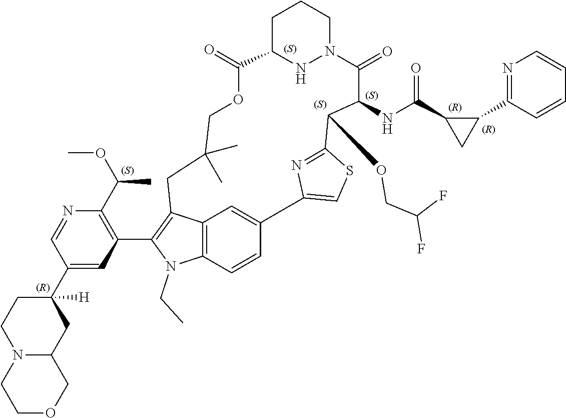 |
| DA 565 | 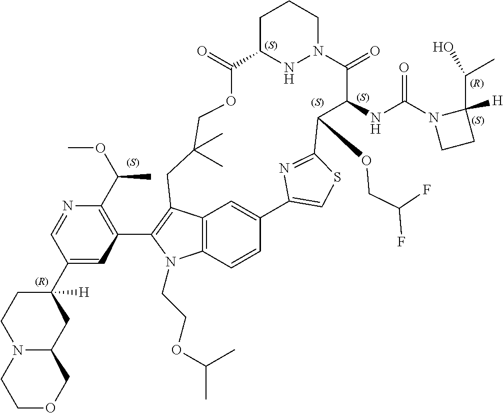 |
| DA 566 | 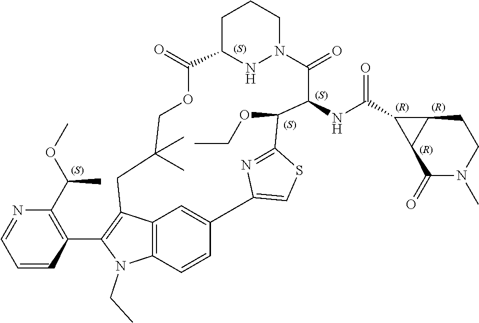 |
| DA 567 | 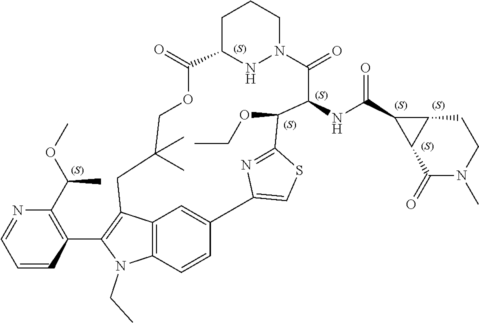 |
| DA 568 | 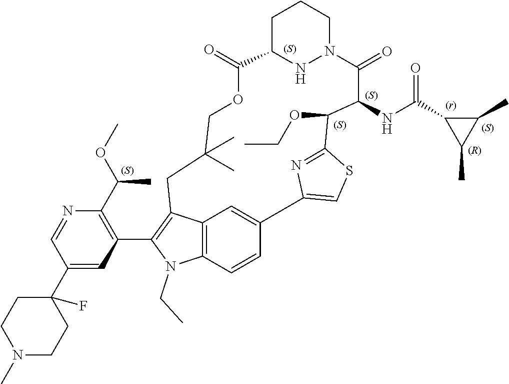 |
| DA 569 | 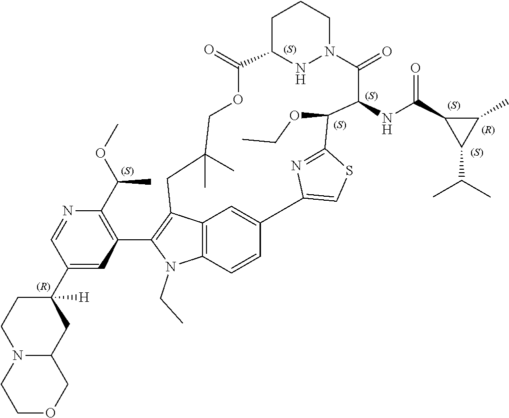 |
| DA 570 | 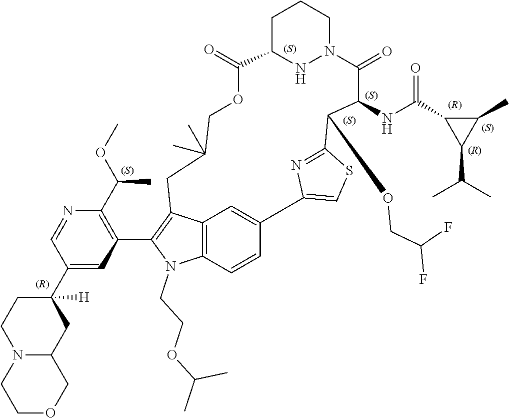 |
| DA 571 | 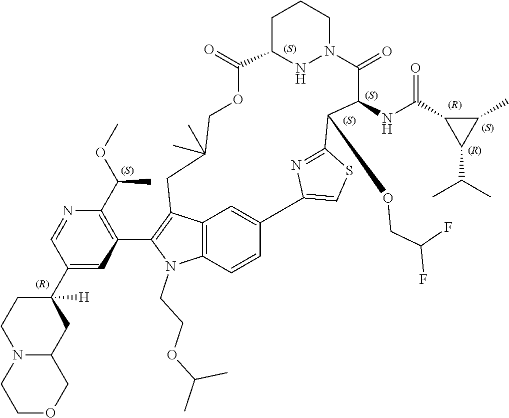 |
| DA 572 | 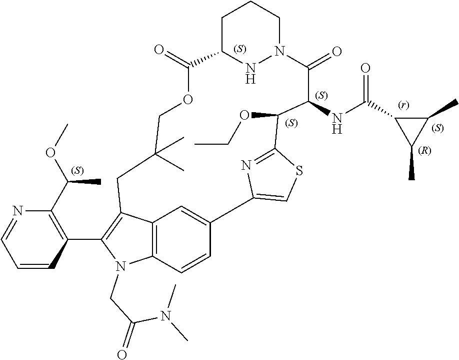 |
| DA 573 | 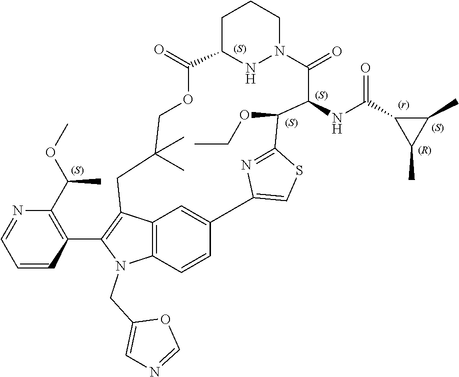 |
| DA 574 | 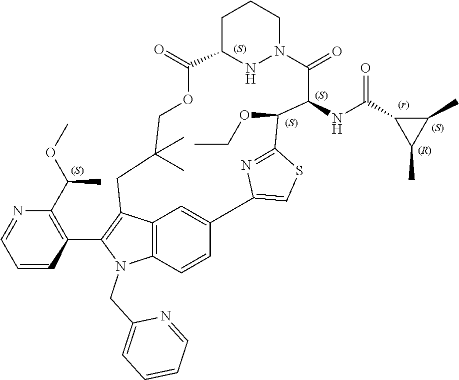 |
| DA 575 | 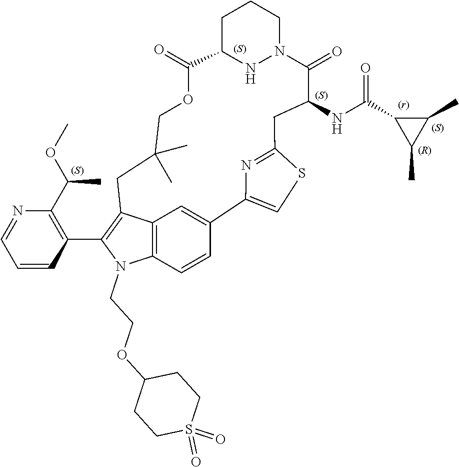 |
| DA 576 | 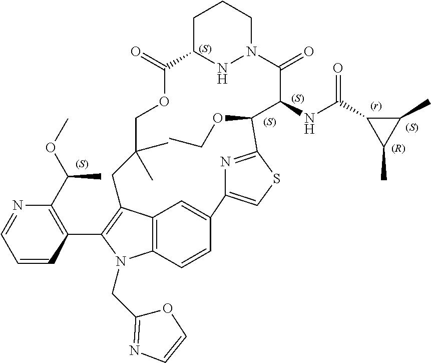 |
| DA 577 | 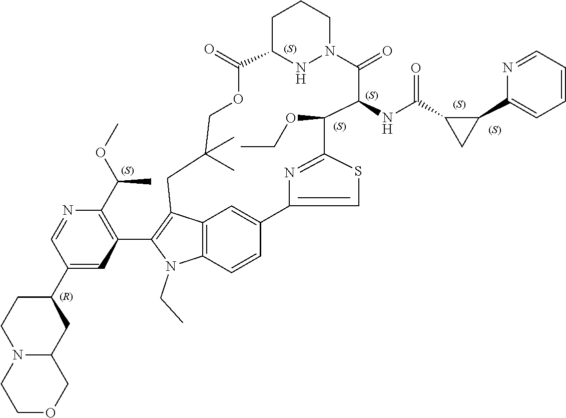 |
| DA 578 | 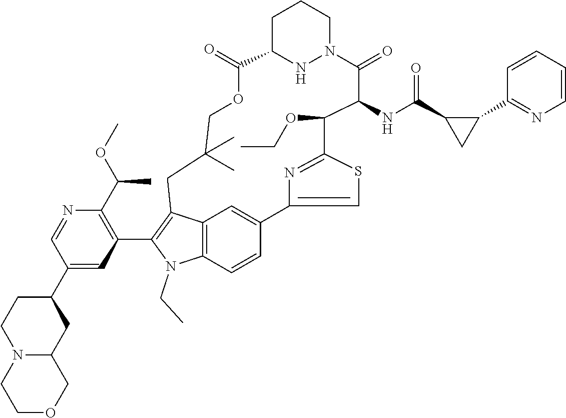 |
| DA 579 | 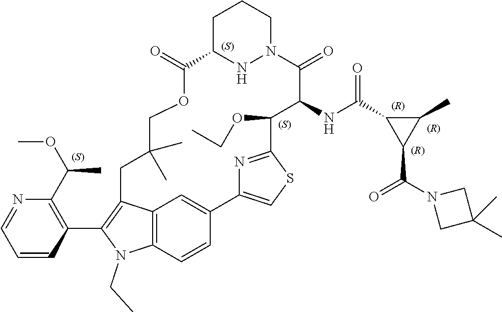 |
| DA 580 | 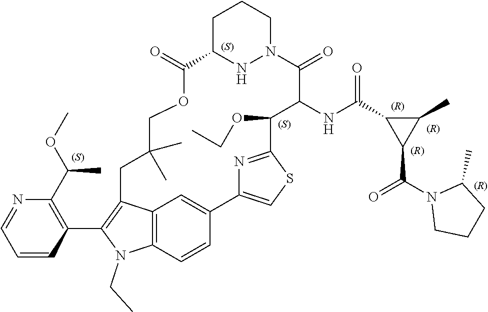 |
| DA 581 | 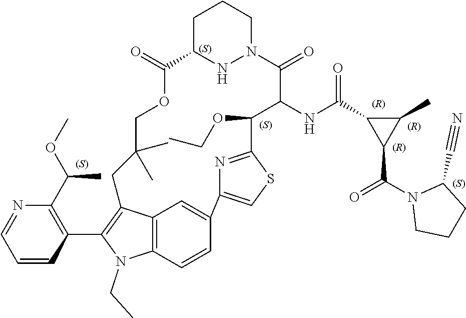 |
| DA 582 | 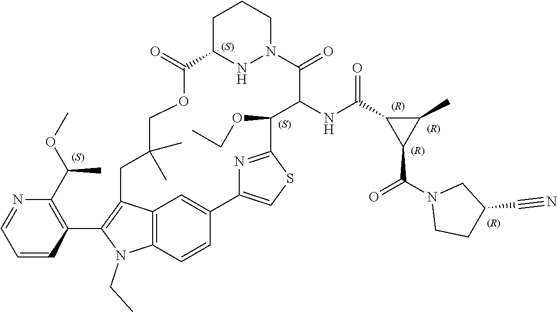 |
| DA 583 | 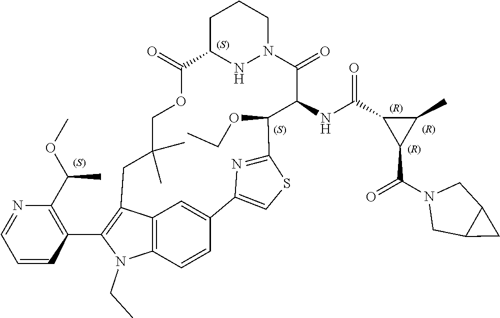 |
| DA 584 | 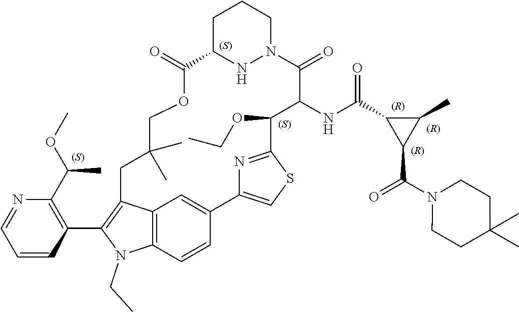 |
| DA 585 | 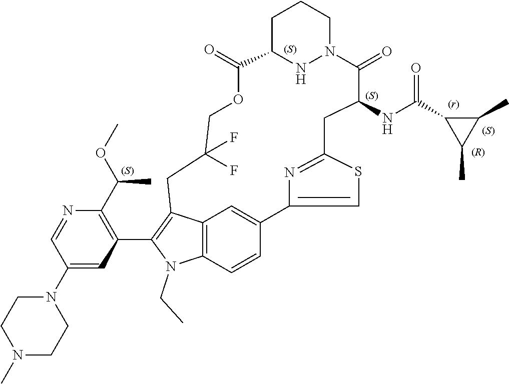 |
| DA 586 | 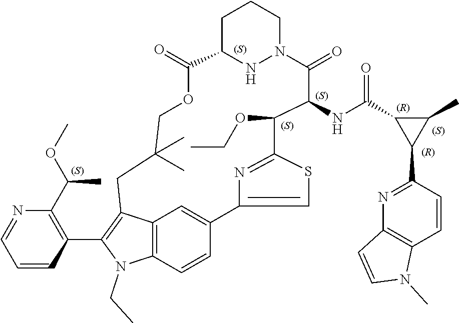 |
| DA 587 | 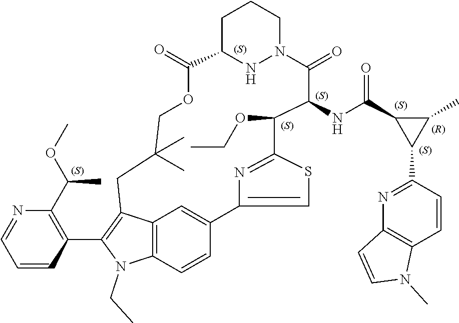 |
| DA 588 | 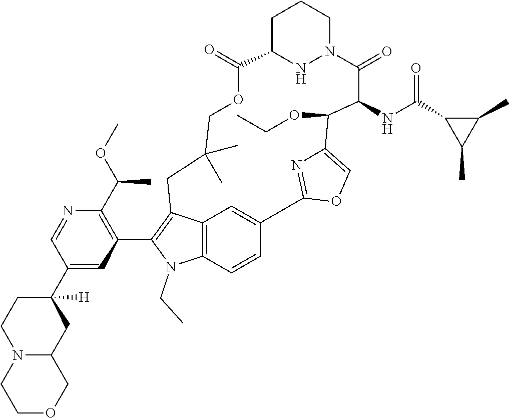 |
| DA 589 | 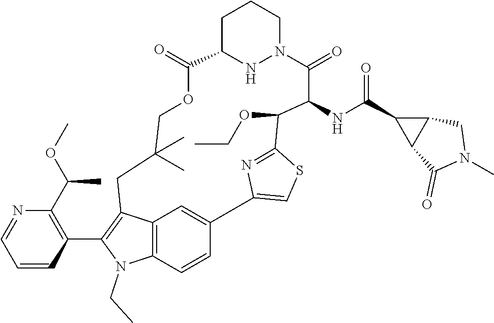 |
| DA 590 | 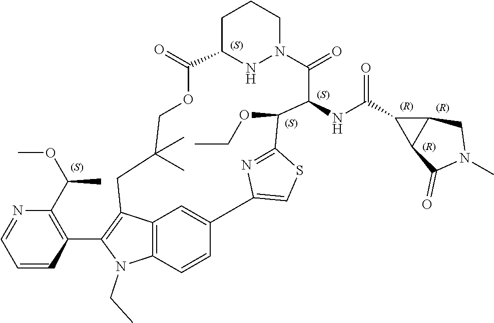 |
| DA 591 | 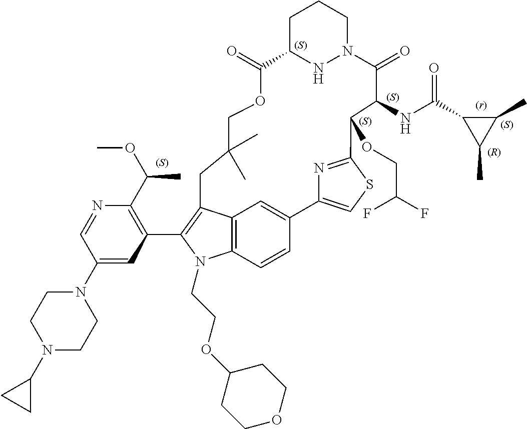 |
| DA 592 | 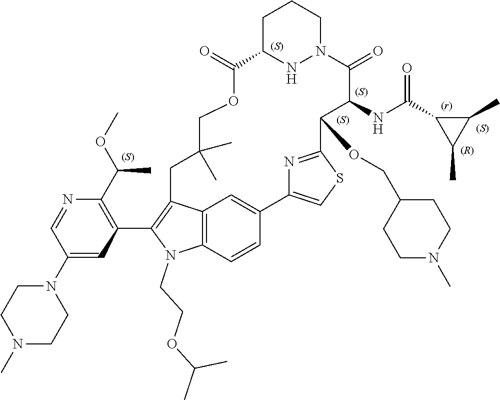 |
| DA 593 | 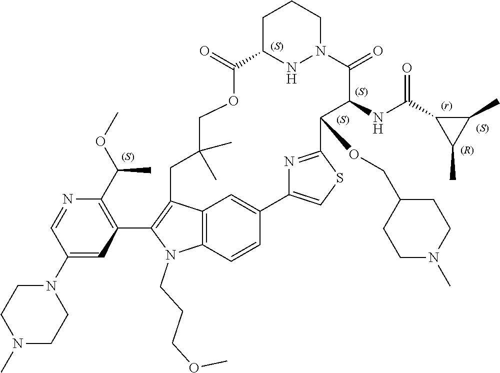 |
| DA 594 | 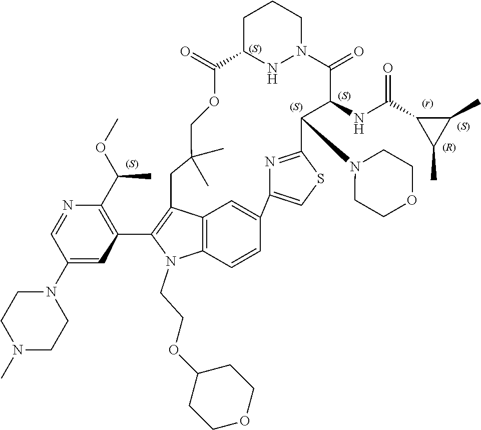 |
| DA 595 | 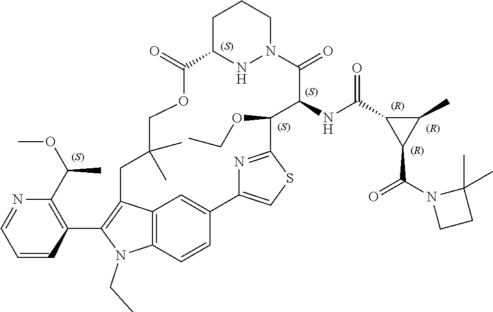 |
| DA 596 | 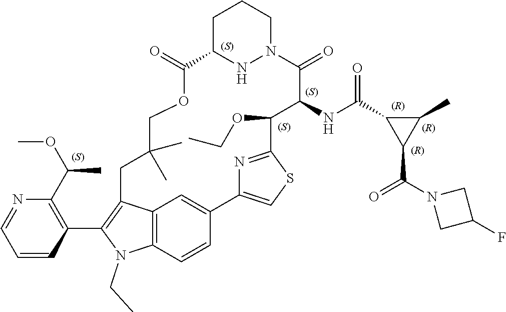 |
| DA 597 | 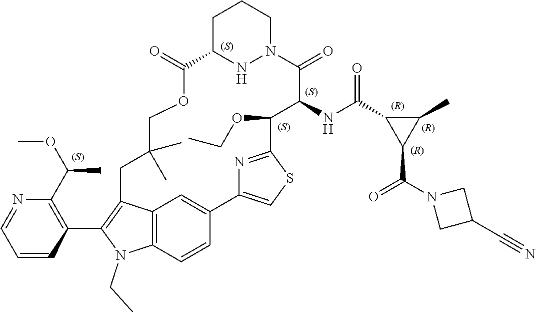 |
| DA 598 | 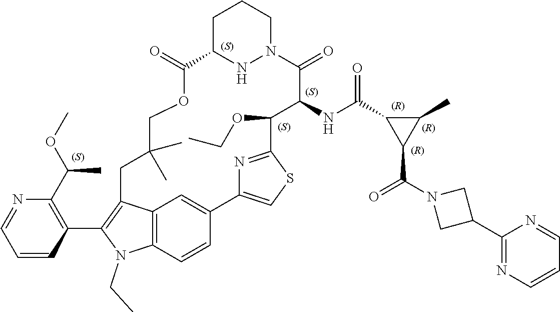 |
| DA 599 | 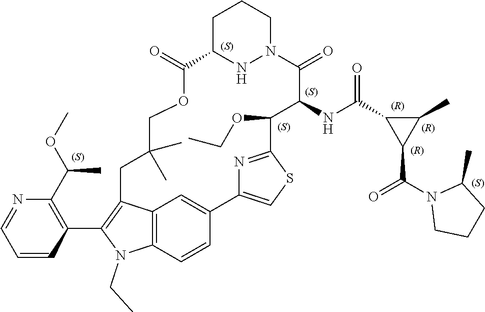 |
| DA 600 | 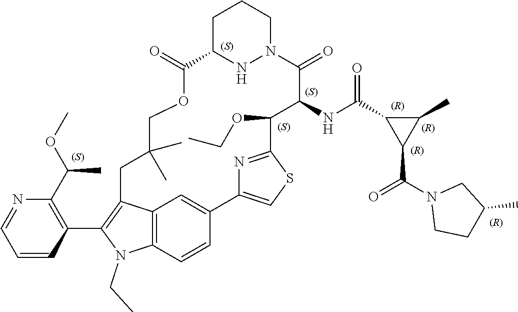 |
| DA 601 | 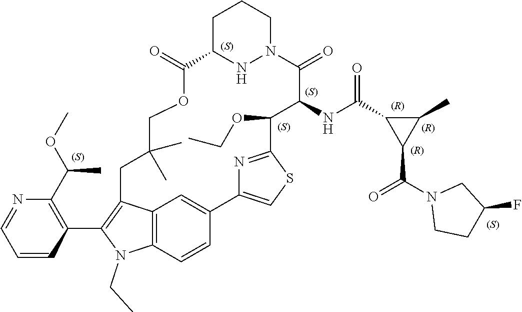 |
| DA 602 | 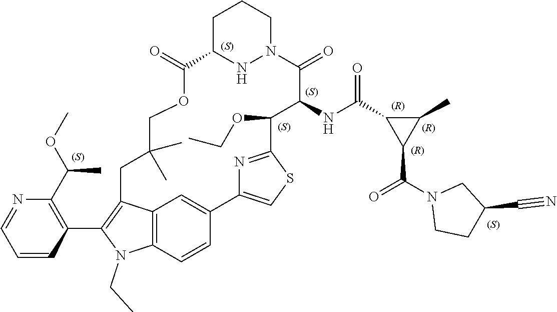 |
| DA 603 | 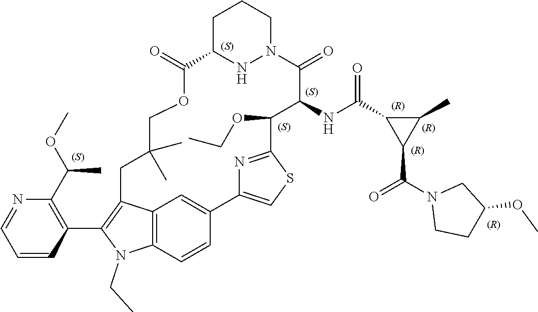 |
| DA 604 | 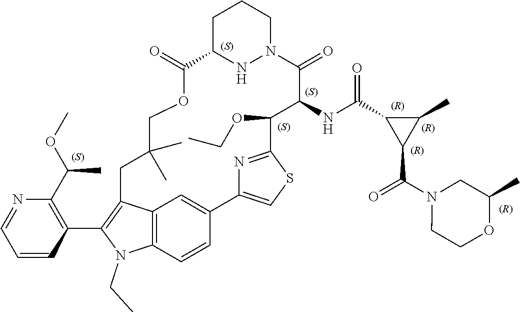 |
| DA 605 | 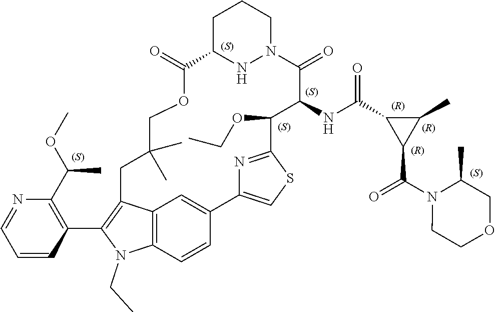 |
| DA 606 | 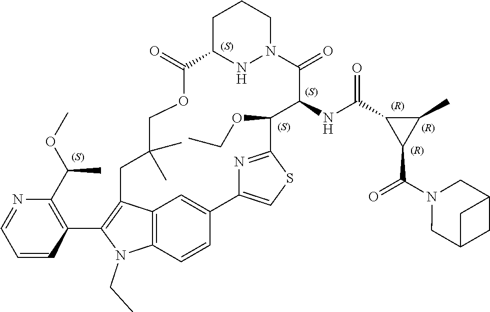 |
| DA 607 | 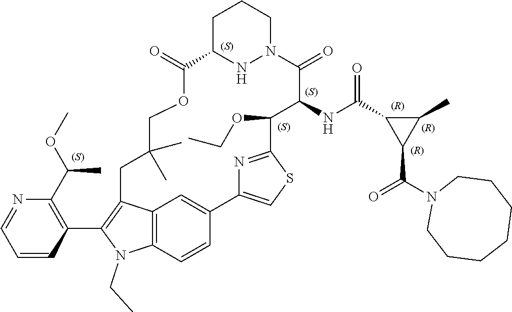 |
| DA 608 | 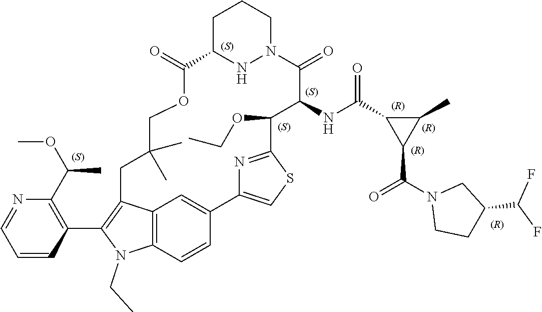 |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated. Any compound shown in brackets indicates that the compound is a diastereomer, and the absolute stereochemistry of such diastereomer may not be known.

In some embodiments, the RAS(MULTI) inhibitor is a compound selected from Table D2, or a pharmaceutically acceptable salt or stereoisomer thereof. In some embodiments, the RAS(MULTI) inhibitor is a compound selected from Table D2, or a pharmaceutically acceptable salt or atropisomer thereof

In some embodiments, the RAS(MULTI) inhibitor is not a compound selected from Table D2. In some embodiments, the RAS(MULTI) inhibitor is not a compound selected from Table D2, or a pharmaceutically acceptable salt or stereoisomer thereof. In some embodiments, the RAS(MULTI) inhibitor is not a compound selected from Table D2, or a pharmaceutically acceptable salt or atropisomer thereof.

TABLE D2
Certain Compounds

| Ex # | Structure |
|---|---|
| DB1 | 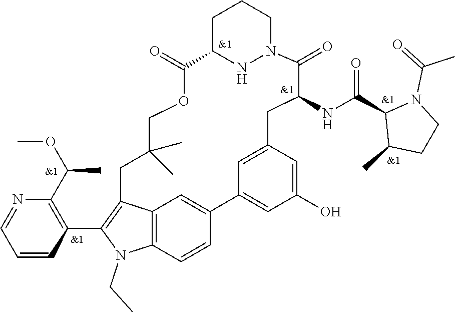 |
| DB2 | 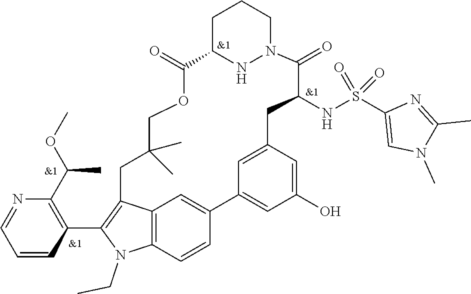 |
| DB3 | 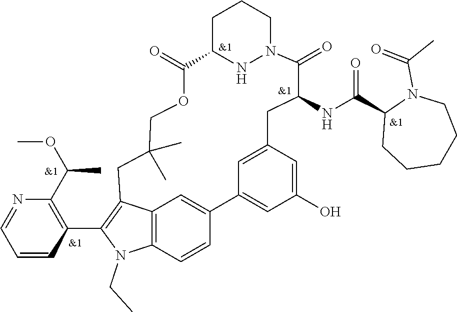 |
| DB4 | 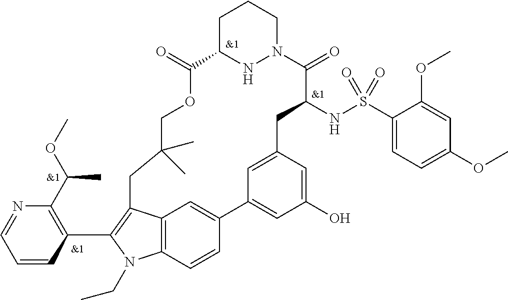 |
| DB5 | 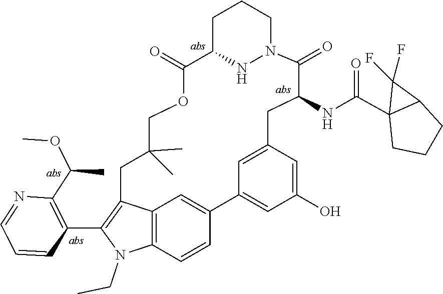 |
| DB6 | 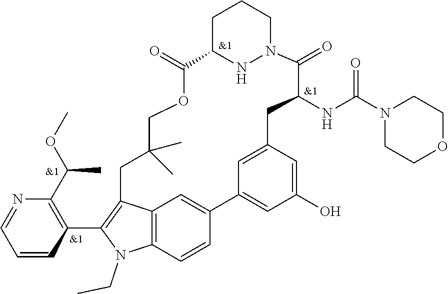 |
| DB7 | 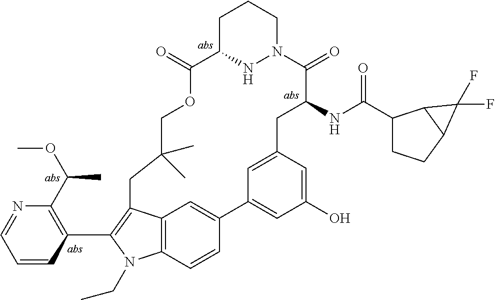 |
| DB8 | 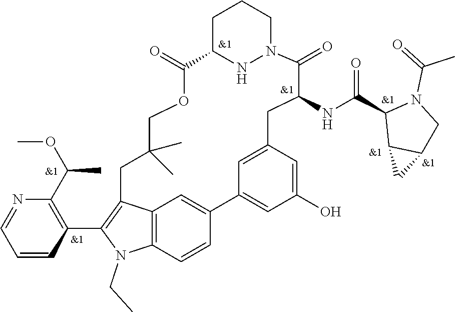 |
| DB9 | 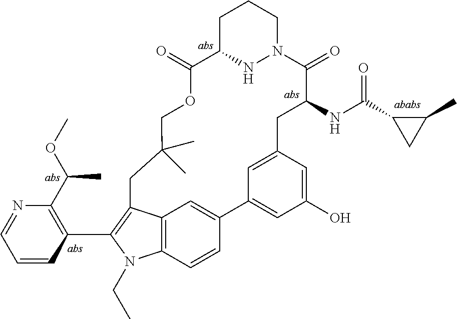 |
| DB10 | 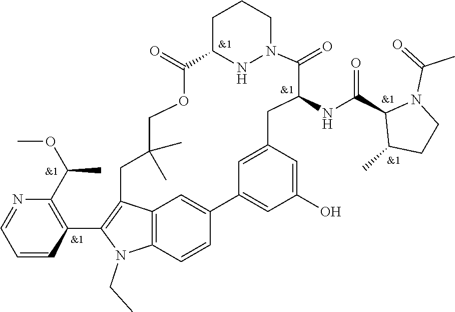 |
| DB11 | 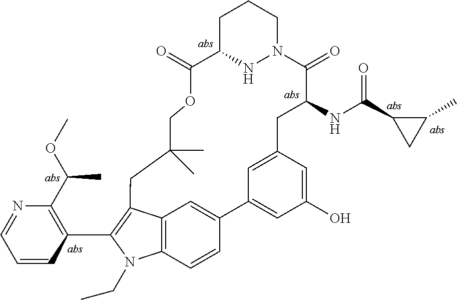 |
| DB12 | 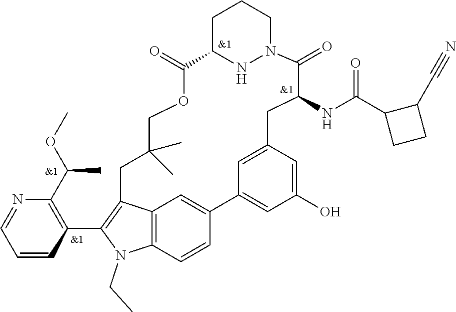 |
| DB13 | 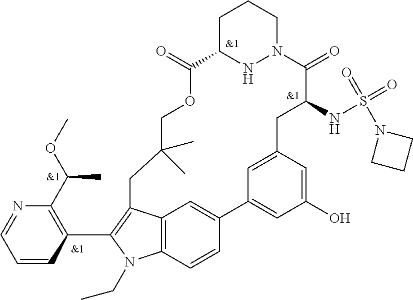 |
| DB14 | 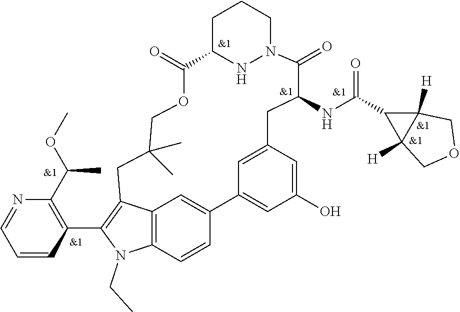 |
| DB15 | 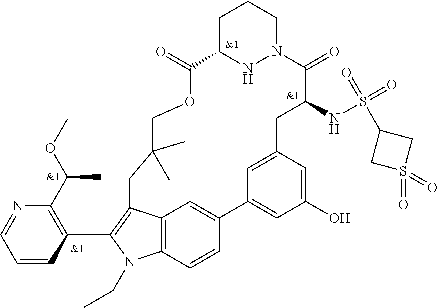 |
| DB16 | 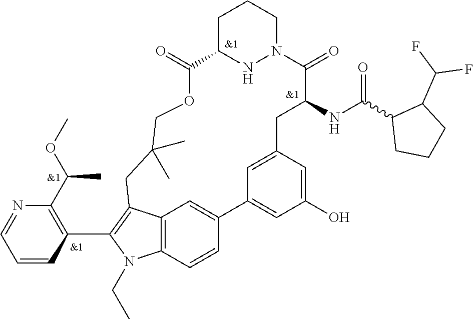 |
| DB17 | 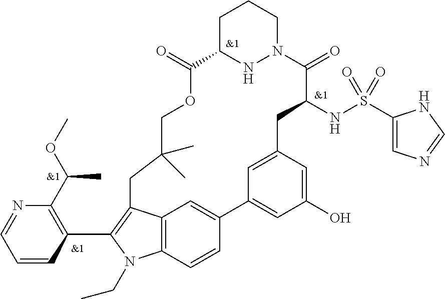 |
| DB18 | 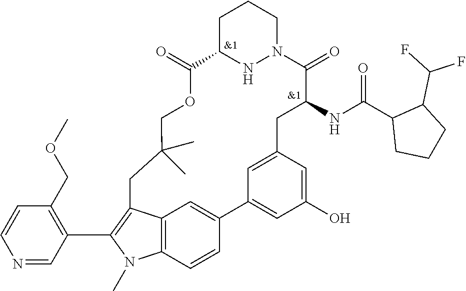 [SMILES: COCc1ccncc1-c1c2c3cc(ccc3n1C)-c1cc(O)cc(c1)C[C@H](NC(=O)C1CCCC1C(F)F)C(=O)N1CCC[C@H](N1)C(=O)OCC(C)(C)C2] |
| DB19 | 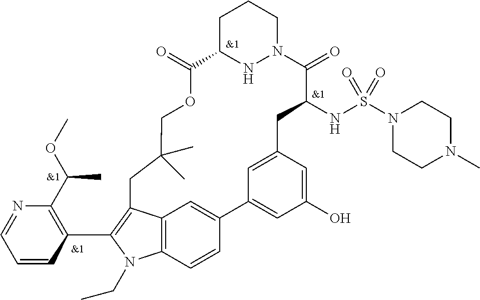 |
| DB20 | 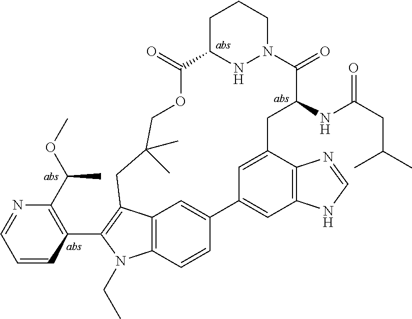 |
| DB21 | 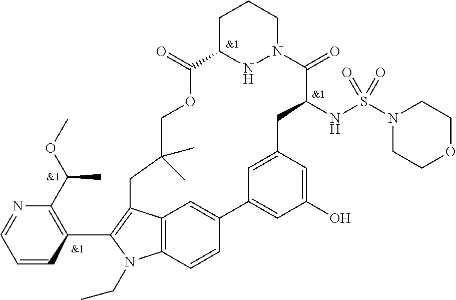 |
| DB22 | 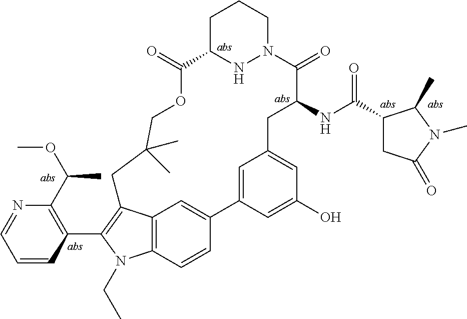 |
| DB23 | 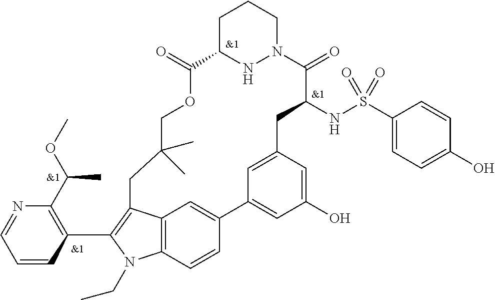 |
| DB24 | 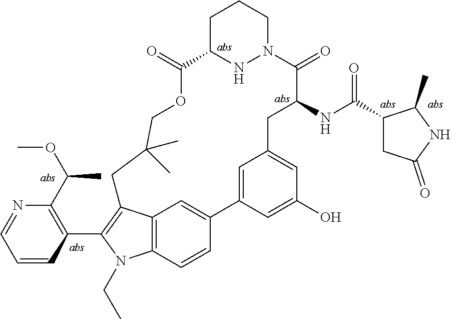 |
| DB25 | 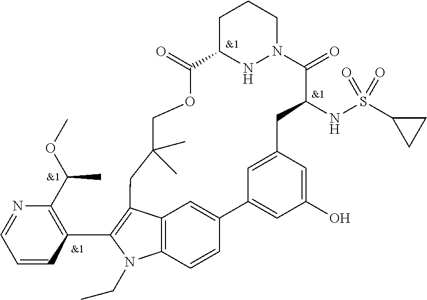 |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated. Any compound shown in brackets indicates that the compound is a diastereomer, and the absolute stereochemistry of such diastereomer may not be known.

In some embodiments, a RAS(MULTI) inhibitor is a compound selected from Table D3 (e.g., DC1-DC20 or DC1-DC21), or a pharmaceutically acceptable salt or stereoisomer thereof. In some embodiments, a compound of the present invention is a compound selected from Table D3 (e.g., DC1-DC20 or DC1-DC21), or a pharmaceutically acceptable salt or atropisomer thereof.

In some embodiments, a compound of the present invention is not a compound selected from Table D3 (e.g., DC1-DC20 or DC1-DC21). In some embodiments, a compound of the present invention is not a compound selected from Table D3 (e.g., DC1-DC20 or DC1-DC21), or a pharmaceutically acceptable salt or stereoisomer thereof. In some embodiments, a compound of the present invention is not a compound selected from Table D3 (e.g., DC1-DC20 or DC1-DC21), or a pharmaceutically acceptable salt or atropisomer thereof.

TABLE D3
Certain Compounds

| Ex. # | Structure |
|---|---|
| DC1 | 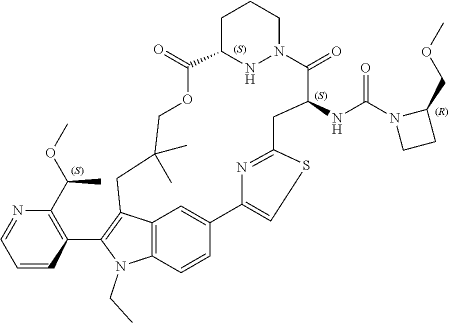 |
| DC2 | 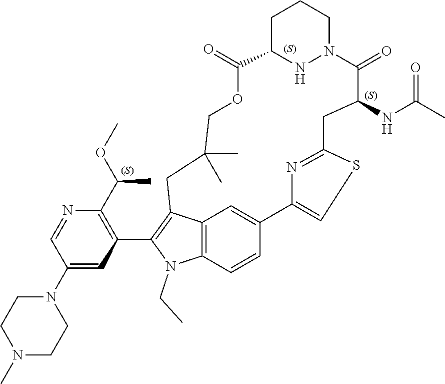 |
| DC3 | 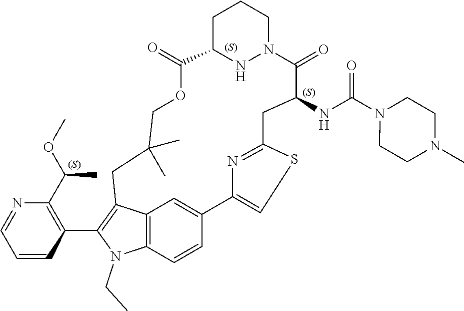 |
| DC4 | 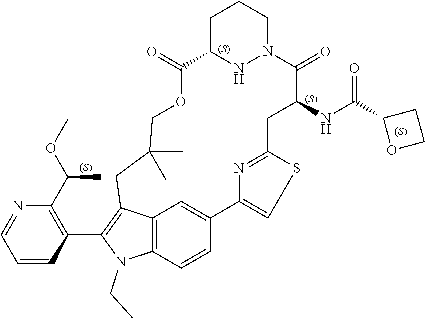 |
| DC5 | 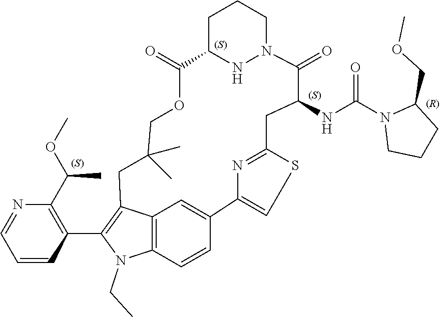 |
| DC6 | 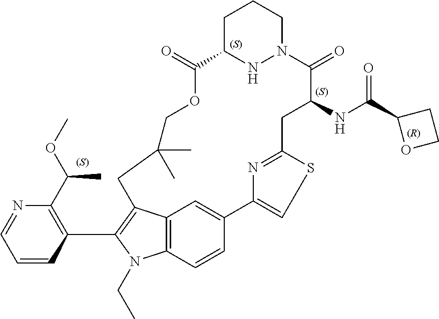 |
| DC7 | 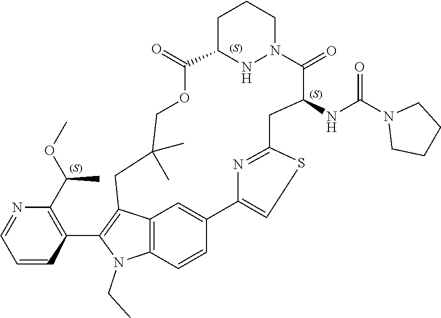 |
| DC8 | 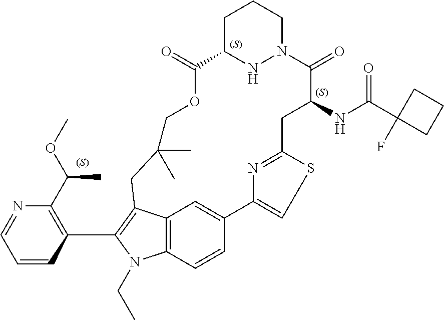 |
| DC9 | 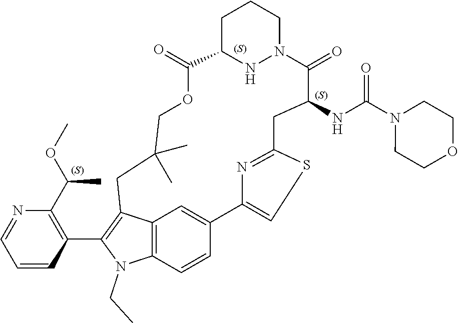 |
| DC10 | 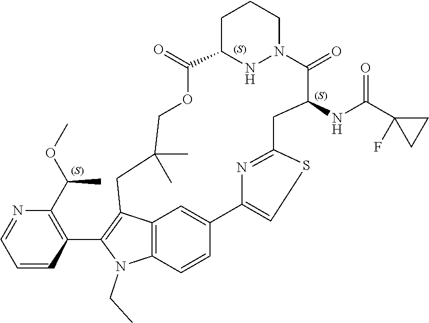 |
| DC11 | 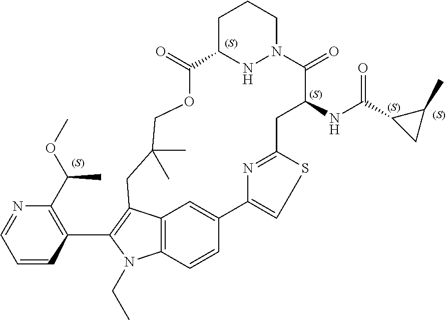 |
| DC12 | 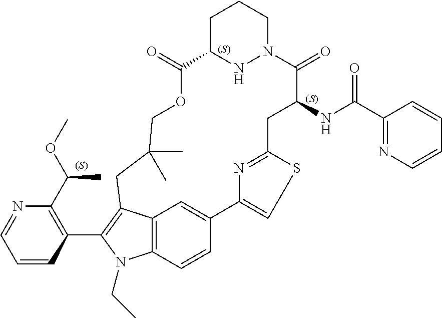 |
| DC13 | 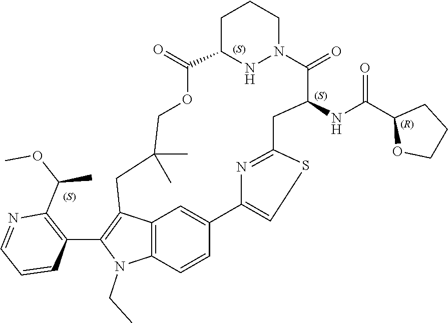 |
| DC14 | 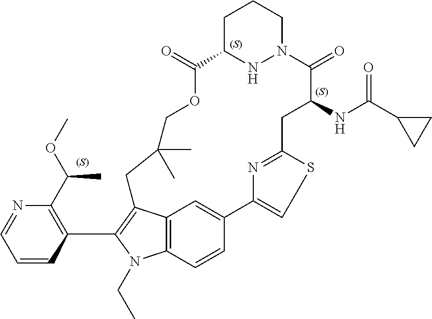 |
| DC15 | 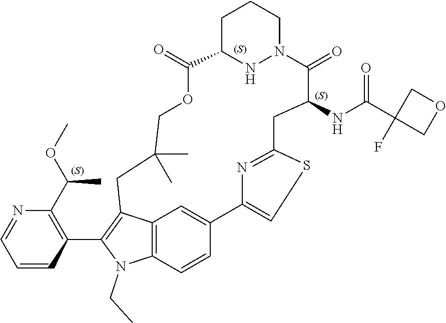 |
| DC16 | 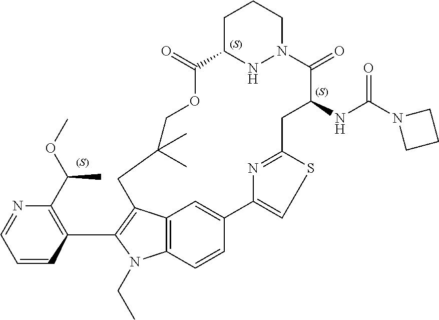 |
| DC17 | 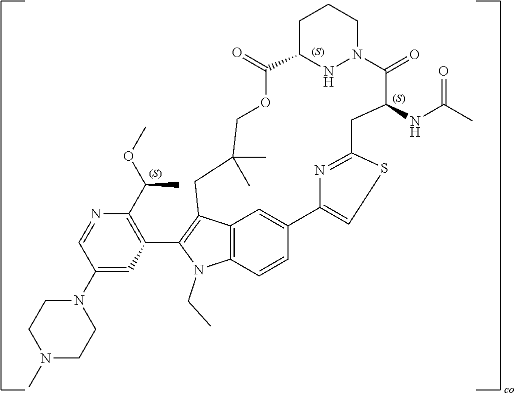 |
| DC18 | 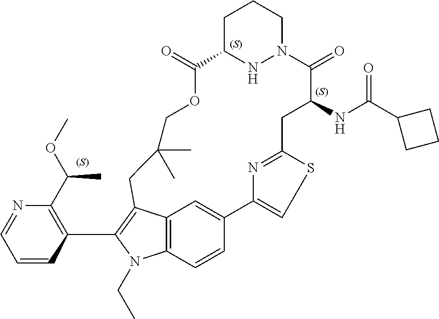 |
| DC19 | 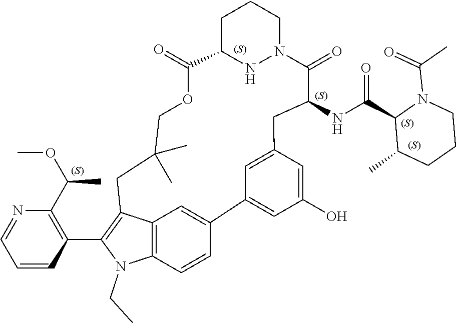 |
| DC20 | 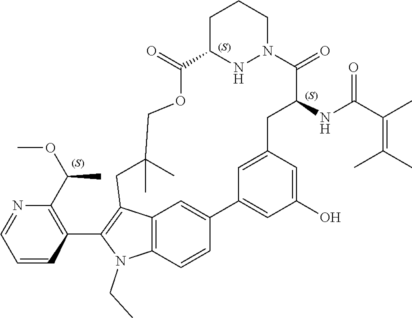 |
| DC21 | 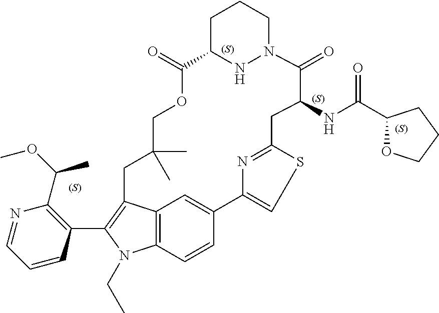 |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated. Any compound shown in brackets indicates that the compound is a diastereomer, and the absolute stereochemistry of such diastereomer may not be known.

The compounds described herein in Tables D1a, D1b, D2, and D3 may be made from commercially available materials or synthesized using known organic, inorganic, or enzymatic processes.

The compounds of the present invention in Tables D1a, D1b, D2, and D3 can be prepared in a number of ways well known to those skilled in the art of organic synthesis. By way example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below and in WO 2022/060836.

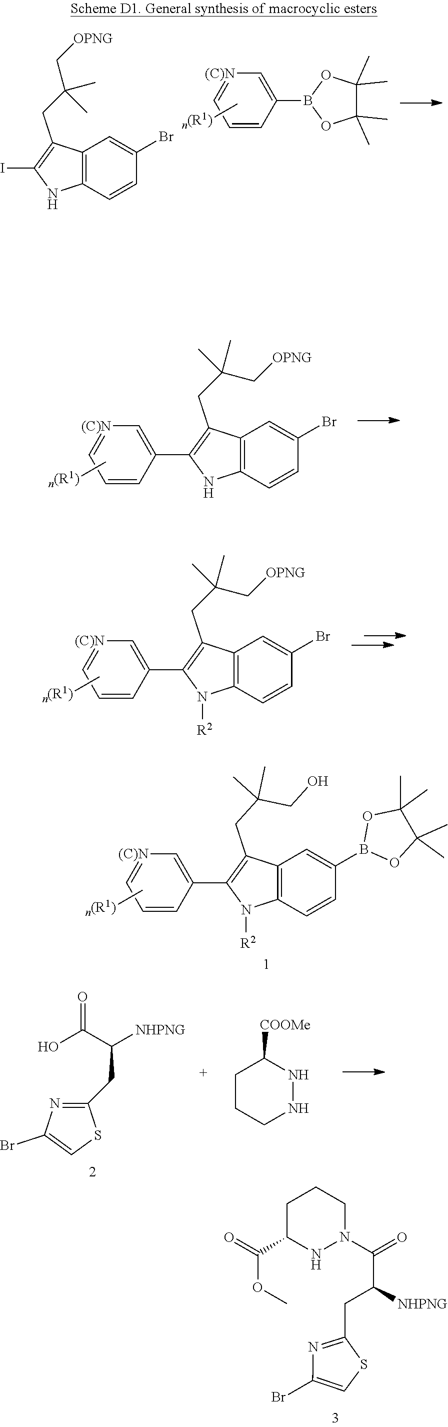

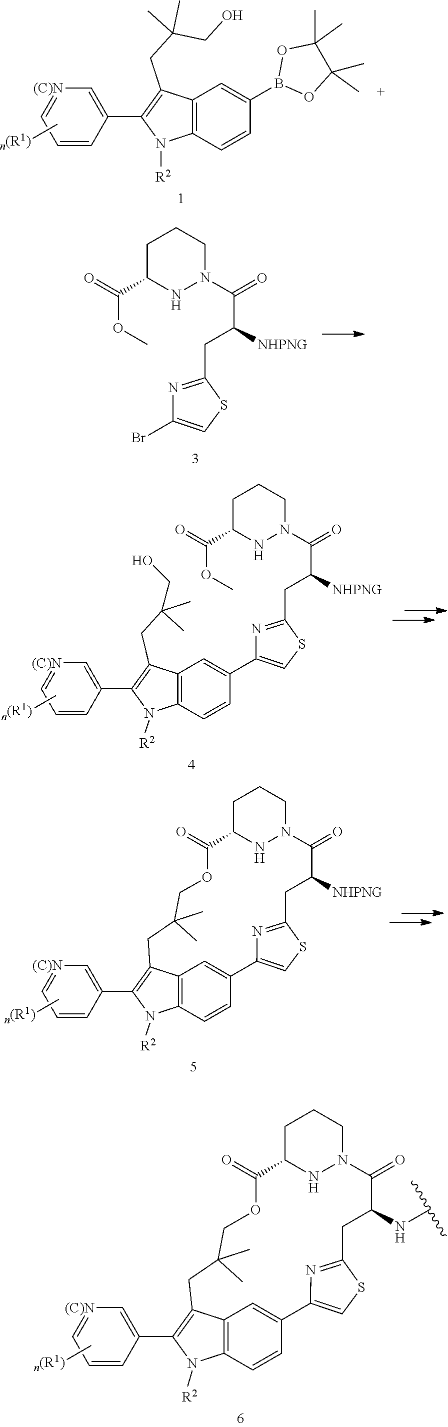

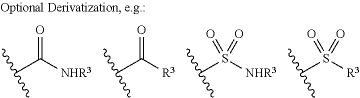

A general synthesis of macrocyclic esters is outlined in Scheme D1. An appropriately substituted indolyl boronic ester (1) can be prepared in four steps starting from protected 3-(5-bromo-2-iodo-1H-indol-3-yl)-2,2-dimethylpropan-1-ol and appropriately substituted boronic acid, including palladium mediated coupling, alkylation, de-protection, and palladium mediated borylation reactions.

Methyl-amino-3-(4-bromothiazol-2-yl)propanoyl)hexahydropyridazine-3-carboxylate (3) can be prepared via coupling of (S)-2-amino-3-(4-bromothiazol-2-yl)propanoic acid (2) with methyl (S)-hexahydropyridazine-3-carboxylate.

The final macrocyclic esters can be made by coupling of methyl-amino-3-(4-bromothiazol-2-yl)propanoyl)hexahydropyridazine-3-carboxylate (3) and an appropriately substituted indolyl boronic ester (1) in the presence of Pd catalyst followed by hydrolysis and macrolactonization steps to result in an appropriately protected macrocyclic intermediate (5). Deprotection and coupling with an appropriately substituted carboxylic acid (or other coupling partner) can result in a macrocyclic product. Additional deprotection or functionalization steps could be required to produce a final compound 6.

Further, with respect to Scheme D1, the thiazole may be replaced with an alternative optionally substituted 5 to 6-membered heteroarylene, or an optionally substituted 3 to 6-membered cycloalkylene, optionally substituted 3 to 6-membered heterocycloalkylene (e.g., morpholino), or optionally substituted 6-membered arylene (e.g., phenyl).

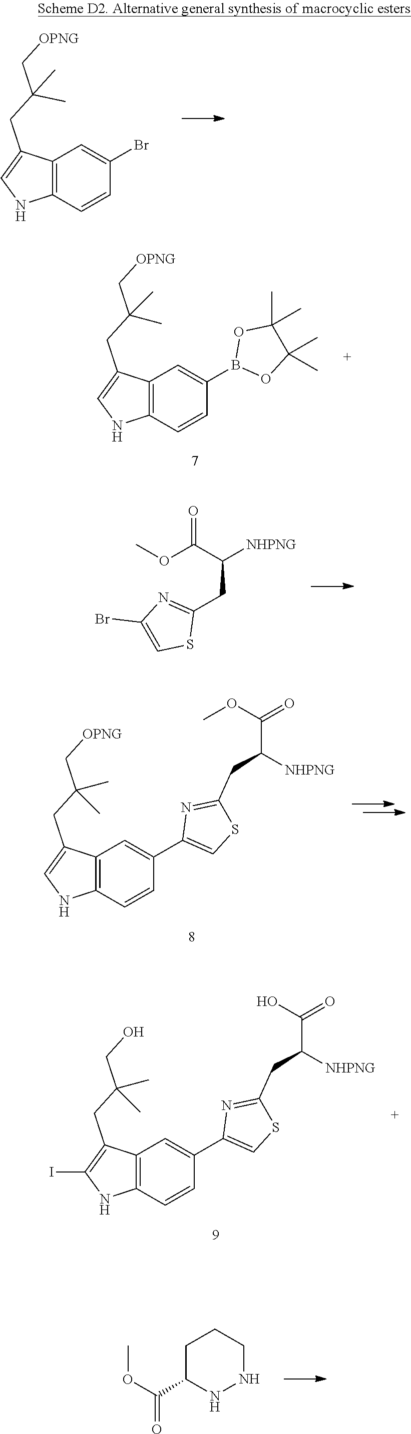

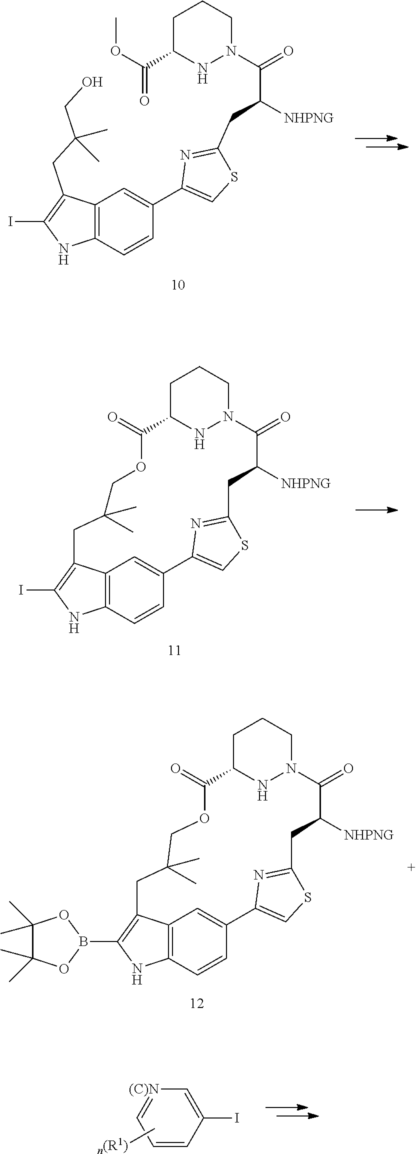

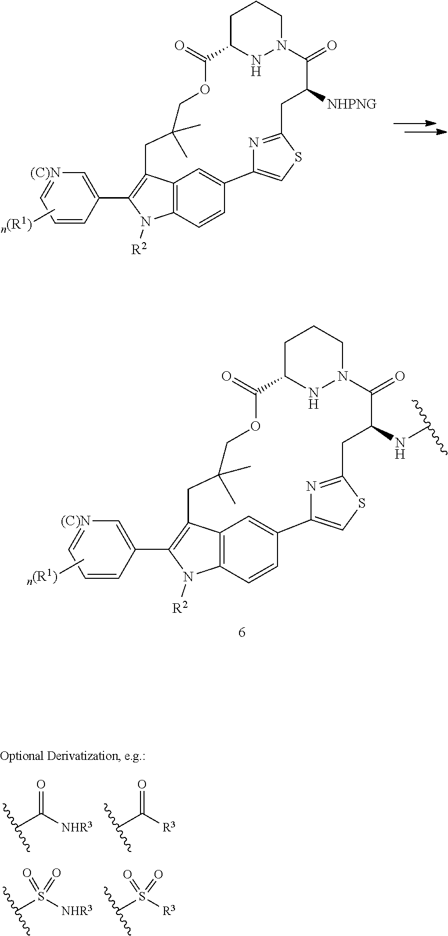

Alternatively, macrocyclic esters can be prepared as described in Scheme D2. An appropriately substituted and protected indolyl boronic ester (7) can be coupled in the presence of Pd catalyst with (S)-2-amino-3-(4-bromothiazol-2-yl)propanoic acid, followed by iodination, deprotection, and ester hydrolysis. Subsequent coupling with methyl (S)-hexahydropyridazine-3-carboxylate, followed by hydrolysis and macrolactonization can result in iodo intermediate (11). Subsequent palladium mediated borylation and coupling in the presence of Pd catalyst with an appropriately substituted iodo aryl or iodo heteroaryl intermediate can yield an appropriately protected macrocyclic intermediate. Alkylation, deprotection and coupling with an appropriately substituted carboxylic acid carboxylic acid (or other coupling partner) results in a macrocyclic product. Additional deprotection or functionalization steps could be required to produce a final compound 6.

Further, with respect to Scheme D2, the thiazole may be replaced with an alternative optionally substituted 5 to 6-membered heteroarylene, or an optionally substituted 3 to 6-membered cycloalkylene, optionally substituted 3 to 6-membered heterocycloalkylene (e.g., morpholino), or optionally substituted 6-membered arylene (e.g., phenyl).

Compounds of Table D1a or Table D1b herein were prepared using methods disclosed in WO 2022/060836 or were prepared using methods described herein combined with the knowledge of one of skill in the art.

### Immune Checkpoint Inhibitors

Compositions and methods described herein may include an immune checkpoint inhibitor (ICI). An immune checkpoint inhibitor may be administered or formulated in combination with a RAS inhibitor described herein. An immune checkpoint inhibitor may be administered or formulated in combination with a RAS inhibitor described herein and a SHP2 inhibitor.

Immune checkpoints refer to a plethora of inhibitory pathways hardwired into the immune system, which, under normal physiological conditions are crucial for maintaining self-tolerance and modulating the duration and amplitude of physiological immune responses in peripheral tissues to minimize collateral tissue damage in response to pathogenic infection. However, the expression of immune checkpoint proteins is often dysregulated by tumors as an important immune resistance and escape mechanism.

Because many of the immune checkpoints are initiated by ligand-receptor interactions, they can be readily blocked by antibodies or modulated by recombinant forms of ligands or receptors. Thus, inhibition of these pathways has been used to activate therapeutic anti-tumor immunity. For example, cytotoxic T-lymphocyte-associated antigen 4 (CTLA-4) antibodies were the first of this class of immunotherapeutics to achieve US Food and Drug Administration (FDA) approval. Preliminary clinical findings with inhibitors of additional immune-checkpoint proteins, such as programmed cell death protein 1 (PD-1), indicate broad and diverse opportunities to enhance anti-tumor immunity with the potential to produce durable clinical responses.

T cell activation through blockade of immune checkpoints has been a major focus of efforts to therapeutically manipulate endogenous anti-tumor immunity, owing to the capacity of T cells for the selective recognition of peptides derived from proteins in all cellular compartments; their capacity to directly recognize and kill antigen-expressing cells (by CD8+ effector T cells; also known as cytotoxic T lymphocytes (CTLs)); and their ability to orchestrate diverse immune responses (by CD4+ helper T cells), which integrate adaptive and innate effector mechanisms. Thus, agonists of co-stimulatory receptors or antagonists of inhibitory signals, both of which result in the amplification of antigen-specific T cell responses, are currently agents of interest in clinical testing.

TABLE 3
Non-limiting list of immune checkpoint targets.

| Target | Biological Function |
|---|---|
| CTLA-4 | Inhibitory Receptor |
| PD-1 | Inhibitory Receptor |
| PD-L1 | Ligand for PD-1 |
| LAG-3 | Inhibitory Receptor |
| B7.1 | Costimulatory Molecule |
| B7-H3 | Inhibitory Ligand |
| B7-H4 | Inhibitory Ligand |
| TIM3 | Inhibitory Receptor |
| VISTA | Inhibitory Receptor |
| CD137 | Costimulatory Molecule |
| OX-40 | Costimulatory Receptor |
| CD40 | Costimulatory Molecule |
| CD27 | Costimulatory Receptor |
| CCR4 | Costimulatory Receptor |
| GITR | Costimulatory Receptor |
| NKG2D | Activating Receptor |
| KIR | Costimulatory Receptor |

CTLA4, cytotoxic T-lymphocyte-associated antigen 4; LAG3, lymphocyte activation gene 3; PD-1, programmed cell death protein 1; PD-L1, PD-1 ligand; TIM3, T cell membrane protein 3; VISTA, V-domain immunoglobulin (Ig)-containing suppressor of T-cell activation; KIR, killer IgG-like receptor.

ICIs approved or in development include, but are not limited to, YERVOY® (ipilimumab), OPDIVO® (nivolumab), KEYTRUDA® (pembrolizumab), tremelimumab, galiximab, MDX-1106, BMS-936558, MEDI4736, MPDL3280A, MEDI6469, BMS-986016, BMS-663513, PF-05082566, IPH2101, KW-0761, CDX-1127, CP-870, CP-893, GSK2831781, MSB0010718C, MK3475, CT-011, AMP-224, MDX-1105, IMP321, and MGA271, as well as numerous other antibodies or fusion proteins directed to the immune checkpoint proteins noted in Table 3. Common immune checkpoint proteins that may be targeted by ICIs include, but are not limited to B7.1, B7-H3, LAG3, CD137, KIR, CCR4, CD27, OX40, GITR, CD40, CTLA4, PD-1, and PD-L1.

In some embodiments, the ICI therapy is selected from one or more of anti-PD-1, anti-PD-L1, anti-CTLA-4, anti-LAG3, anti-B7.1, anti-B7H3, anti-B7H4, anti-TIM3, anti-VISTA, anti-CD137, anti-OX40, anti-CD40, anti-CD27, anti-CCR4, anti-GITR, anti-NKG2D, and anti-KIR. In some embodiments, the ICI therapy is an antibody (e.g., a monoclonal antibody selective for any of the targets in Table 3). In some embodiments the ICI is an anti-PD-1 antibody.

### SHP2 Inhibitors

Compositions and methods described herein may include a SHP2 inhibitor. A SHP2 inhibitor may be administered or formulated in combination with a RAS inhibitor described herein. A SHP2 inhibitor may be administered or formulated in combination with a RAS inhibitor and an immune checkpoint inhibitor.

SHP2 is a non-receptor protein tyrosine phosphatase encoded by the PTPN11 gene that contributes to multiple cellular functions including proliferation, differentiation, cell cycle maintenance and migration. SHP2 has two N-terminal Src homology 2 domains (N—SH2 and C—SH2), a catalytic domain (PTP), and a C-terminal tail. The two SH2 domains control the subcellular localization and functional regulation of SHP2. The molecule exists in an inactive, self-inhibited conformation stabilized by a binding network involving residues from both the N—SH2 and PTP domains. Stimulation by, for example, cytokines or growth factors acting through receptor tyrosine kinases (RTKs) leads to exposure of the catalytic site resulting in enzymatic activation of SHP2.

SHP2 is involved in signaling through the RAS-mitogen-activated protein kinase (MAPK), the JAK-STAT or the phosphoinositol 3-kinase-AKT pathways. Mutations in the PTPN11 gene and subsequently in SHP2 have been identified in several human developmental diseases, such as Noonan Syndrome and Leopard Syndrome, as well as human cancers, such as juvenile myelomonocytic leukemia, neuroblastoma, melanoma, acute myeloid leukemia and cancers of the breast, lung and colon. Some of these mutations destabilize the auto-inhibited conformation of SHP2 and promote autoactivation or enhanced growth factor driven activation of SHP2. SHP2, therefore, represents a highly attractive target for the development of novel therapies for the treatment of various diseases including cancer. A SHP2 inhibitor (e.g., RMC-4550 or SHP099) in combination with a RAS pathway inhibitor (e.g., a MEK inhibitor) has been shown to inhibit the proliferation of multiple cancer cell lines in vitro (e.g., pancreas, lung, ovarian and breast cancer).

Non-limiting examples of such SHP2 inhibitors that are known in the art, include: Chen et al. Mol Pharmacol. 2006, 70, 562; Sarver et al., J. Med. Chem. 2017, 62, 1793; Xie et al., J. Med. Chem. 2017, 60, 113734; and Igbe et al., Oncotarget, 2017, 8, 113734; and PCT applications: WO 2022033430, WO 2022017444, WO 2022007869, WO 2021259077, WO 2021249449, WO 2021249057, WO 2021244659, WO 2021218755, WO 2021281752, WO 2021149817, WO 2021148010, WO 2021147879, WO 2021143823, WO 2021143701, WO 2021143680, WO 2021121397, WO 2021119525, WO 2021115286, WO 2021110796, WO 2021088945, WO 2021073439, WO 2021061706, WO 2021061515, WO 2021043077, WO 2021033153, WO 2021028362, WO 2021033153, WO 2021028362, WO 2021018287, WO 2020259679, WO 2020249079, WO 2020210384, WO 2020201991, WO 2020181283, WO 2020177653, WO 2020165734, WO 2020165733, WO 2020165732, WO 2020156243, WO 2020156242, WO 2020108590, WO 2020104635, WO 2020094104, WO 2020094018, WO 2020081848, WO 2020073949, WO 2020073945, WO 2020072656, WO 2020065453, WO 2020065452, WO 2020063760, WO 2020061103, WO 2020061101, WO 2020033828, WO 2020033286, WO 2020022323, WO 2019233810, WO 2019213318, WO 2019183367, WO 2019183364, WO 2019182960, WO 2019167000, WO 2019165073, WO 2019158019, WO 2019152454, WO 2019051469, WO 2019051084, WO 2018218133, WO 2018172984, WO 2018160731, WO 2018136265, WO 2018136264, WO 2018130928, WO 2018129402, WO 2018081091, WO 2018057884, WO 2018013597, WO 2017216706, WO 2017211303, WO 2017210134, WO 2017156397, WO 2017100279, WO 2017079723, WO 2017078499, WO 2016203406, WO 2016203405, WO 2016203404, WO 2016196591, WO 2016191328, WO 2015107495, WO 2015107494, WO 2015107493, WO 2014176488, WO 2014113584, US 20210085677, U.S. Ser. No. 10/858,359, U.S. Ser. No. 10/934,302, U.S. Ser. No. 10/954,243, U.S. Ser. No. 10/988,466, U.S. Ser. No. 11/001,561, U.S. Ser. No. 11/033,547, U.S. Ser. No. 11/034,705, or U.S. Ser. No. 11/044,675, or a pharmaceutically acceptable salt, solvate, isomer (e.g., stereoisomer), prodrug, or tautomer thereof, each of which is incorporated herein by reference.

In some embodiments, a SHP2 inhibitor binds in the active site. In some embodiments, a SHP2 inhibitor is a mixed-type irreversible inhibitor. In some embodiments, a SHP2 inhibitor binds an allosteric site e.g., a non-covalent allosteric inhibitor. In some embodiments, a SHP2 inhibitor is a covalent SHP2 inhibitor, such as an inhibitor that targets the cysteine residue (C333) that lies outside the phosphatase's active site. In some embodiments a SHP2 inhibitor is a reversible inhibitor. In some embodiments, a SHP2 inhibitor is an irreversible inhibitor. In some embodiments, the SHP2 inhibitor is SHP099. In some embodiments, the SHP2 inhibitor is TNO155, having the structure

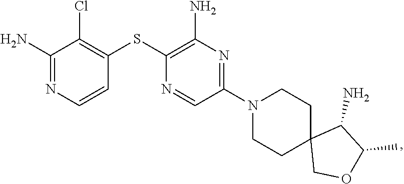 [SMILES: C[C@@H]1OCC2(CCN(c3cnc(Sc4ccnc(N)c4Cl)c(N)n3)CC2)[C@@H]1N]

or a pharmaceutically acceptable salt, solvate, isomer (e.g., stereoisomer), prodrug, or tautomer thereof. In some embodiments, the SHP2 inhibitor is RMC-4550, having the structure

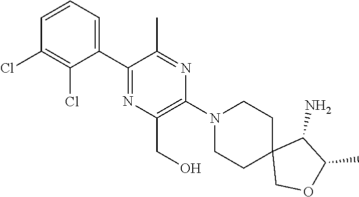 [SMILES: Cc1nc(N2CCC3(CC2)CO[C@@H](C)[C@H]3N)c(CO)nc1-c1cccc(Cl)c1Cl]

or a pharmaceutically acceptable salt, solvate, isomer (e.g., stereoisomer), prodrug, or tautomer thereof.
In some embodiments, the SHP2 inhibitor is RMC-4630, having the structure

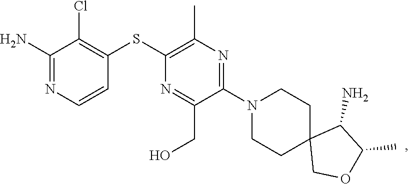 [SMILES: Cc1nc(N2CCC3(CC2)CO[C@@H](C)[C@H]3N)c(CO)nc1Sc1ccnc(N)c1Cl]

or a pharmaceutically acceptable salt, solvate, isomer (e.g., stereoisomer), prodrug, or tautomer thereof. In some embodiments, the SHP2 inhibitor is JAB-3068, having the structure

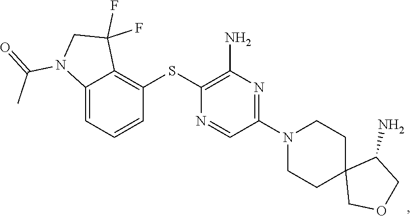 [SMILES: CC(=O)N1CC(F)(F)c2c(Sc3ncc(N4CCC5(CC4)COC[C@H]5N)nc3N)cccc21]

or a pharmaceutically acceptable salt, solvate, isomer (e.g., stereoisomer), prodrug, or tautomer thereof. In some embodiments, the SHP2 inhibitor is JAB-3312. In some embodiments, the SHP2 inhibitor is RLY-1971, having the structure

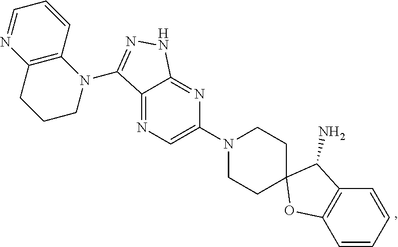 [SMILES: N[C@@H]1c2ccccc2OC12CCN(c1cnc3c(N4CCCc5ncccc54)n[nH]c3n1)CC2]

or a pharmaceutically acceptable salt, solvate, isomer (e.g., stereoisomer), prodrug, or tautomer thereof. In some embodiments, the SHP2 inhibitor is ERAS-601, or a pharmaceutically acceptable salt, solvate, isomer (e.g., stereoisomer), prodrug, or tautomer thereof. In some embodiments, the SHP2 inhibitor is BBP-398, or a pharmaceutically acceptable salt, solvate, isomer (e.g., stereoisomer), prodrug, or tautomer thereof.

### Methods of Use

In some embodiments, the invention discloses a method of treating a subject having cancer by administering to the subject a compound or combination of compounds described herein. In some embodiments, the subject is administered a RAS inhibitor described herein (i.e., a RAS G12C inhibitor). In some embodiments, the subject is administered a first RAS inhibitor (e.g., a RAS G12C inhibitor), a second RAS inhibitor (e.g., a RAS(MULTI) inhibitor), and a SHP2 inhibitor. In some embodiments, the subject is also administered an immune checkpoint inhibitor1. In some embodiments, the cancer is an immune refractory cancer (e.g., immune refractory lung cancer).

RAS inhibitors described herein may sensitize immune refractory cancers to immunotherapy. For example, RAS inhibitors described herein may promote the transformation of immunologically cold tumors to immunologically hot tumors, which in turn enables the treatment of immunologically cold tumors. Additionally, a RAS inhibitor or a combination of compounds described herein can boost the efficacy of other cancer therapies when administered in combination to the subject. It is suggested herein that therapies using a RAS inhibitor or a combination of compounds described herein, can alter the tumor immune infiltrate comprising of T-cells, B-cells, APCs, monocytes, MDSCs, TAMs, neutrophils, other monocyte-derived cells, tumor-associated stroma, cancer stem cells, and mesenchymal stem cells and result in an enhanced anti-tumor therapeutic effect.

### Identification of Tumor Types

In various embodiments, the disclosure provides a method of treating lung cancer in a subject comprising administering to the subject a RAS inhibitor or combination of compounds described herein, wherein the subject has one or more tumors that are characterized as immune refractory, immune evasive, immunologically protected, immunologically “cold,” microsatellite stable, microsatellite instability low, comprising a low immune infiltrate, comprising a low tumor mutational burden, or exhibiting heterogeneity.

In various embodiments, the disclosure provides a method for treating tumors that are characterized as immune evasive, immunologically protected, immunologically “cold,” microsatellite stable, microsatellite instability low, comprising a low immune infiltrate, comprising a low tumor mutational burden or exhibiting heterogeneity in a subject, comprising (i) diagnosing the subject as having an immune refractory, immune evasive tumor, immunologically protected tumor, immunologically “cold” tumor, microsatellite stable tumor, microsatellite instability low tumor, a tumor comprising a low immune infiltrate, a tumor comprising a low tumor mutational burden or a tumor exhibiting heterogeneity, and (ii) administering a RAS inhibitor or combination of compounds described herein to the subject. In various embodiments, the diagnosing comprises assaying biomarkers/characteristics associated with tumors that are characterized as immune refractory, immune evasive, immunologically protected, immunologically “cold,” microsatellite stable, microsatellite instability low, comprising a low immune infiltrate, comprising a low tumor mutational burden or exhibiting heterogeneity. In various embodiments, the method further comprises (iii) determining if the subject's tumor becomes immune responsive, and then (iv) administering an immunotherapy, optionally in combination with the RAS inhibitor or combination of compounds described herein.

Also provided herein is a method for determining if a subject is or is likely to be responsive to treatment with a RAS inhibitor or combination of compounds described herein, and treating the subject accordingly. In various embodiments, a patient diagnosed with cancer undergoes testing to identify the tumor as a cold tumor, e.g., using methods described herein and others described in the art. The disclosure provides a method for treating a subject having a cancer, e.g., an immune refractory cancer, with a RAS inhibitor or combination of compounds described herein, the method comprising obtaining a tumor sample from a subject, conducting assays to determine if the tumor is a cold tumor, and treating the subject with a RAS inhibitor or combination of compounds described herein if the tumor is identified as a cold tumor. Assays to determine whether the tumor is a cold tumor include, but are not limited to tumor mutational burden analysis, microsatellite instability (MSI) testing, the degree of immune cell (e.g., CD4⁺ T-cells, CD8⁺ T-cells, NK1.1⁺ NK cells, APCs, monocytes, and neutrophils) infiltration into the tumor, immune cell phenotype (e.g., PD-1⁺, PD-L1⁺, and PD-L2⁺), immune cell function (e.g., expression of IFN-γ, IL-12, IL-15, and MHCII), and ratio of pro-inflammatory and anti-inflammatory mediators in the tumor microenvironment (TME).

Multiple diagnostic tools designed to characterize tumors at the cellular and molecular level are FDA-approved and commercially available. Examples of approved diagnostics include FOUNDATIONONE® CDX, FOUNDATIONONE® LIQUID, FOUNDATIONON® HEME, BRACAnalysis CDx, therascreen EGFR RGQ PCR kit, cobase EGFR Mutation Test v2, PD-L1 IHC 22C3 pharmDx, Abbott RealTime IDH1, MRDx BCR-ABL test, VENTANA ALK (D5F3) CDx Assay, Abbott RealTime IDH2, Praxis Extended RAS Panel, Oncomine Dx Target Test, LeukoStrat CDx FLT3 Mutation Assay, FoundationFocus CDxBRCA Assay, Vysis CLL FISH Probe Kit, KIT D816V Mutation Detection, PDGFRB FISH, cobas KRAS Mutation Test, therascreen KRAS RGQ PCR Kit, FerriScan, Dako c-KIT pharmDx, INFORM Her-2/neu, PathVysion HER-2 DNA Probe Kit, SPOT-LIGHT HER2 CISH Kit, Bond Oracle HER2 IHC System, HER2 CISH pharmDx Kit, INFORM HER2 DUAL ISH DNA Probe Cocktail, HercepTest, HER2 FISH pharmDx Kit, THXID BRAF Kit, Vysis ALK Break Apart FISH Probe Kit, cobas 4800 BRAF V600 Mutation Test, VENTANA PD-L1 (SP142) Assay, therascreen FGFR RGQ RT-PCR Kit, and therascreen PIK3CA RGQ PCR Kit.

In various embodiments, the subject is screened for eligibility for treatment with one or more immunotherapies described herein. In various embodiments, subjects that are not eligible for treatment with such immunotherapies may be first treated with a RAS inhibitor or combination of compounds described herein according to the methods described herein. Non-limiting examples of immunotherapies include Pembrolizumab (KEYTRUDA®, Merck Sharp & Dohme Corp), Nivolumab (OPDIVO®, Bristol-Myers Squibb), Atezolizumab (TECENTRIQ®), Avelumab (BAVENCIO®), and Durvalumab (IMFINZI®). Eligibility criteria for these immunotherapies are known in the art. For example, without limitation, pembrolizumab (KEYTRUDA®), nivolumab (OPDIVO®), and atezolizumab (TECENTRIQ®) have eligibility criteria based on PD-L1 expression levels. PD-L1 expression criteria and methods of measuring the same may be found at keytrudahcp.com/biomarker-testing/pd-11-expression-testing/(pembrolizumab; KEYTRUDA®), or the FDA-approved prescribing information for pembrolizumab (KEYTRUDA®, as revised January 2020), atezolizumab (e.g., TECENTRIQ®, as revised May 2020), and nivolumab (e.g., OPDIVO®, as revised on June 2020). Each of these publications are herein incorporated by reference in its entirety for all purposes. As described herein, treating such patients with a RAS inhibitor or combination of compounds described herein may promote the transformation of the tumor that is not eligible for treatment with an immunotherapy to an immunogenic tumor, which in turn will enable such tumors to be treated with an immunotherapy. In various embodiments, the tumors of subjects that are not eligible for an immunotherapy can be monitored throughout the course of treatment with a RAS inhibitor or combination of compounds described herein in order to determine when the tumor becomes eligible for treatment with an immunotherapy. Once the tumor is eligible for treatment with an immunotherapy, the subject may be administered an immunotherapy, alone or in combination with a RAS inhibitor or a SHP2 inhibitor, or a combination thereof.

In various embodiments, the disclosure provides a method of treating lung cancer in a subject comprising administering to the subject a RAS inhibitor or combination of compounds described herein, wherein the subject has one or more tumors with a low immune infiltrate. In various embodiments, the administering to a subject with one or more lung tumors with a low immune infiltrate alters the tumor immune infiltrate. In various embodiments, the tumor immune infiltrate comprises antigen-presenting cells, myeloid cells, and lymphoid cells. In various embodiments, antigen-presenting cells in the tumor immune infiltrate comprise macrophages or dendritic cells. In various embodiments, myeloid cells in the tumor immune infiltrate comprise monocytes, neutrophils, myeloid-derived suppressor cells (MDSCs), and tumor-associated macrophages (TAMs). In various embodiments, the TAMs in the tumor immune infiltrate comprise M1 macrophages, M2 macrophages, and MARCO⁺ macrophages. In various embodiments, lymphoid cells in the tumor immune infiltrate comprise T-cells, B-cells, NK T-cells, and NK cells.

Qualitative and quantitative methods have been described for the characterization of the tumor immune infiltrate, including but not limited, microscopic analyses, histological assays, cytological assays, flow cytometry, polymerase chain reaction (PCR), quantitative polymerase chain reaction (qPCR), RNA sequencing (RNA-seq), single-cell RNA sequencing (scRNA-seq), next-generation sequencing, whole-exome sequencing, epigenetic sequencing, ATAC-seq, microarray analysis, and mass cytometry or CyTOF. Biomarkers can be used, alone or in combination, for the evaluation of immune cells and include cell surface markers and secreted proteins. Exemplary biomarkers for the characterization of the tumor immune infiltrate include, but are not limited to, CD45, CD3, CD4, CD8, CD25, CD44, CD134, CD252, CD137, CD79, CD39, FOXP3, PD-1, LAG-3, TIM-1, IFN-γ, Granzyme, Perforin, CD11b, CD11c, Ly6C, Ly6G, CD14, CD16, CD80, MARCO, CD68, CD115, CD206, CD163, CD103c, F4/80, PD-L1, PD-L2, Arginase, iNOS, ROS, TNF-α, TGF-β, MHC-I, MHC-II, NK1.1, NKG2D, CD244, Ki67, CD19, CD20, CCR2, CXCR3, CCR4, CCR5, CCR6, CCR7, CCR10, CCL2, CCL5, Cx3CR1, CCL10, ICOS, CD40, CD40L, IL1α, IL1β, IL2, IL4, IL5, IL6, IL8, IL12, IL15, IL17, IL21, IL22, TCRγ/δ, TCRα/β, STAT3, ROR1c, and RORγt.

Cancer stem cells (CSCs) have been described as a subset of cells found within solid and hematologic tumors that are tumorigenic, and capable of self-renewal, differentiation. Several reports have described the importance of CSCs in the pathogenesis of a variety of tumors, tumor relapse after therapy, and development of therapeutic resistance. A number of cell surface markers can be used to distinguish CSCs within solid and hematologic tumors. CSC markers include, but are not limited to, CD19, CD20, CD24, CD34, CD38, CD44, CD90, CD133, Aldehyde dehydrogenase 1, CEACAM-6/CD66c, BMI-1, Connexin 43/GJA1, DLL4, EpCAM/TROP1, GLI-1, GLI-2, Integrins, PON1, PTEN, ALCAM/CD166, DPPIV/CD26, Lgr5, Musashi-1, A20, ABCG2, CD15, Fractalkine, HIF-2α, L1CAM, c-MAF, Nestin, Podoplanin, SOX2, CD96, CD117, FLT3, AFP, CD13, CD90, NF2/Merlin, ABCB5, NGFR, Syndecan-1, Endoglin, STRO-1, and PON1.

In various embodiments, the disclosure provides a method of treating cancer in a subject comprising administering to the subject a RAS inhibitor or combination of compounds described herein, wherein the subject has one or more immune refractory tumors. In various embodiments, the subject has one or more immunologically protected tumors. In various embodiments, the subject has one or more microsatellite stable tumors. In various embodiments, the subject has one or more microsatellite low tumors. In various embodiments, the subject has one or more tumors with moderate microsatellite instability. In various embodiments, the subject has one or more tumors with a low tumor mutational burden. In various embodiments, the subject has one or more tumors with a moderate tumor mutational burden. In various embodiments, the subject has one or more tumors resistant to therapy. In various embodiments, the subject has one or more immunologically heterogeneous tumors. In various embodiments, the subject has genetically heterogeneous tumors. In various embodiments, the subject has one or more refractory tumors. In one or more embodiments, the subject has a tumor that develops resistance during the course of treatment.

In various embodiments, the tumor characteristic is determined from one or more biological samples from a subject suffering from cancer. In various embodiments, the tumor characteristic is determined by comparing one or more biological samples from a subject suffering from cancer to one or more biological samples from one or more healthy subjects. In various embodiments, the tumor characteristic is determined from one or more biological samples selected from the group consisting of blood, cerebrospinal fluid, urine, stool, buccal swab, nasal swab, lavage, tissue biopsy, bone marrow biopsy, and tumor biopsy. In various embodiments, the tumor characteristic is determined from the analysis of cells, proteins, or nucleic acids in one or more biological samples from a subject suffering from cancer. In various embodiments, the tumor characteristic is determined by comparing the analysis of cells, proteins, or nucleic acids in one or more biological samples from a subject suffering from cancer to the analysis of one or more biological samples from one or more healthy subjects. In various embodiments, the tumor characteristic is determined by comparing the analysis of cells, proteins, or nucleic acids in one or more biological samples from a subject suffering cancer to the analysis of one or more biological samples from one or more subjects suffering from cancer and responsive to treatment. In various embodiments, the cells are selected from the group consisting of leukocytes, epithelial cells, mesenchymal cells, mesenchymal stem cells, stromal cells, endothelial cells, fibroblasts, cancer-associated fibroblasts (CAFs), pericytes, adipocytes, cancer stem cells, circulating tumor cells (CTCs), hematopoietic stem cells, and hematopoietic progenitor cells. In various embodiments, the proteins are selected from the group consisting of cytokines, chemokines, growth factors, signal transduction proteins, enzymes, proteases, and nucleases. In various embodiments, the nucleic acids are selected from the group consisting of DNA, ssDNA, circulating tumor DNA (ctDNA), RNA, mRNA, dsRNA, siRNA, miRNA, and lncRNA. In various embodiments, the nucleic acid analysis is performed by PCR, RT-PCR, qRT-PCR, next-generation sequencing (NGS), RNA-seq, ATAC-seq, exome sequencing, Southern Blot, microarray analysis, or single-cell sequencing.

In various embodiments, the tumor characteristic of a subject suffering from cancer is determined from the analysis of one or more blood samples collected from the subject. In various embodiments, the tumor characteristic of a subject suffering from cancer is determined from the analysis of cells, proteins, or nucleic acids in one or more blood samples collected from the subject. In various embodiments, the tumor characteristic of a subject suffering from cancer is determined by comparing the analysis of cells, proteins, or nucleic acids in one or more blood samples from the subject suffering from cancer to the analysis of one or more blood samples from one or more healthy subjects. In various embodiments, the cells analyzed in one or more blood samples are leukocytes, epithelial cells, mesenchymal cells, mesenchymal stem cells, stromal cells, endothelial cells, fibroblasts, cancer associated fibroblasts (CAFs), pericytes, adipocytes, cancer stem cells, circulating tumor cells (CTCs), hematopoietic stem cells, and hematopoietic progenitor cells. In various embodiments, the leukocytes are myeloid cells and lymphoid cells. In various embodiments, myeloid cells are monocytes, macrophages, neutrophils, granulocytes, dendritic cells, mast cells, eosinophils, and basophils. In various embodiments, the lymphoid cells are T cells, B cells, NK cells, NK-T cells, or iNK cells.

In various embodiments, the analysis of cells from one or more blood samples collected from a subject suffering from cancer demonstrates increased levels of immune suppressive cells compared to the analysis of cells from one or more healthy subjects or one or more subjects suffering from cancer and responsive to treatment. In various embodiments, the immune suppressive cells are myeloid derived suppressor cells (MDSCs), tumor-associated macrophages (TAMs), neutrophils, T_{reg} cells, and B_{reg} cells. In various embodiments, MDSCs are monocytic MDSCs (M-MDSCs) and polymorphonuclear MDSCs (PMN-MDSCs). In various embodiments, the TAMs are M2 TAMs. In various embodiments, the immune suppressive cells are CAFs. In various embodiments, the levels of immune suppressive cells in one or more blood samples of a subject having one or more tumors characterized as immune refractory, immunologically protected, or immunologically “cold” are increased by about 5-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% compared to one or more blood samples collected from one or more healthy subjects or one or more subjects suffering from cancer and responsive to treatment. In various embodiments, the levels of immune suppressive cells in one or more blood samples of a subject having one or more tumors characterized as immune refractory, immunologically protected, or immunologically “cold” are increased by about 2-100 fold (e.g., about 2, 5, 10, 15, 20, 25, 30, 35, 40, 45, 50, 55, 60, 65, 70, 75, 80, 85, 90, 95, or 100 fold, inclusive of all values and ranges between these values) compared to one or more blood samples collected from one or more healthy subjects or one or more subjects suffering from cancer and responsive to treatment. In various embodiments, immune suppressive cells are identified by the assay of cell-surface proteins expression. In various embodiments, the analysis of cells from one or more blood samples collected from a subject having one or more tumors characterized as immune refractory, immunologically protected, or immunologically “cold” demonstrates reduced levels or absence of activated pro-inflammatory immune cells (e.g., reduced relative to a healthy subject or a subject suffering from cancer and responsive to treatment by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values).

In various embodiments, the analysis of cells from one or more blood samples collected from a subject having one or more tumors characterized as immune refractory, immunologically protected, or immunologically “cold” demonstrates reduced levels or absence of activated pro-inflammatory immune cells (e.g., reduced relative to a healthy subject or a subject suffering from cancer and responsive to treatment by about 2, 5, 10, 15, 20, 25, 30, 35, 40, 45, 50, 55, 60, 65, 70, 75, 80, 85, 90, 95, or 100 fold inclusive of all values and ranges between these values). In various embodiments, the activated pro-inflammatory cells are dendritic cells (DCs), macrophages, M1 macrophages, T-cells, B-cells, NK cells, NK-T cells, and iNK cells. In various embodiments, the frequency of pro-inflammatory immune cells is ≤10% (e.g., about 9%, about 8%, about 7%, about 6%, about 5%, about 4%, about 3%, about 2%, or about 1%) of all leukocytes analyzed from one or more blood samples collected from the subject. In various embodiments, activated pro-inflammatory immune cells are identified by the assay of cell-surface protein expression.

In various embodiments, the analysis of cells in one or more blood samples of a subject suffering from cancer is performed by the assay of cell-surface proteins. In various embodiments, the cell-surface proteins are selected from the group consisting of receptor tyrosine kinase (RTK), CD1c, CD2, CD3, CD4, CD5, CD8, CD9, CD10, CD11b, CD11c, CD14, CD15, CD16, CD18, CD19, CD20, CD21, CD22, CD23, CD24, TACI, CD25, CD27, CD28, CD30, CD30L, CD31, CD32, CD32b, CD34, CD33, CD38, CD39, CD40, CD40-L, CD41b, CD42a, CD42b, CD43, CD44, CD48, CD47, CD45RA, CD45RO, CD48, CD52, CD55, CD56, CD58, CD61, CD66b, CD70, CD72, CD79, CD68, CD84, CD86, CD93, CD94, CD95, CRACC, BLAME, BCMA, CD103, CD107, CD112, CD120a, CD120b, CD123, CD125, CD134, CD135, CD140a, CD141, CD154, CD155, CD160, CD163, CD172a, XCR1, CD203c, CD204, CD206, CD207 CD226, CD244, CD267, CD268, CD269, CD355, CD358, NKG2A, NKG2B, NKG2C, NKG2D, NKG2E, NKG2F, NKG2H, KIR2DL1, KIR2DL2, KIR2DL3, KIR2DL5A, KIR2DL5B, KIR3DL1, KIR3DL2, KIR3DL3, KIR3DL4, KIR2DS1, KIR2DS2, KIR2DS3, KIR2DS4, KIR2DS5, DAP12, KIR3DS, NKp44, NKp46, TCR, BCR, Integrins, FcβεRI, MHC-I, MHC-II, IL-1R, IL-2Rα, IL-2Rβ, IL-2Rγ, IL-3Rα, CSF2RB, IL-4R, IL-5Rα, IL-6Rα, gp130, IL-7Rα, IL-9R, IL-12Rβ1, IL-12Rβ2, IL-13Rα1, IL-13Rα2, IL-15Rα, IL-21R, IL-23R, IL-27Rα, IL-31Rα, OSMR, CSF-1R, cell-surface IL-15, IL-10Rα, IL-10Rβ, IL-20Rα, IL-20Rβ, IL-22Rα1, IL-22Rα2, IL-22Rβ, IL-28RA, PD-1, PD-1H, BTLA, CTLA-4, PD-L1, PD-L2, 2B4, B7-1, B7-2, B7-H1, B7-H4, B7-DC, DR3, LIGHT, LAIR, LTα1β2, LTβR, TIM-1, TIM-3, TIM-4, TIGIT, LAG-3, ICOS, ICOS-L, SLAM, SLAMF2, OX-40, OX-40L, GITR, GITRL, TL1A, HVEM, 41-BB, 41BB-L, TL-1A, TRAF1, TRAF2, TRAF3, TRAF5, BAFF, BAFF-R, APRIL, TRAIL, RANK, AITR, TRAMP, CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6, CXCR7, CLECL9a, DC-SIGN, IGSF4A, SIGLEC, EGFR, PDGFR, VEGFR, FAP, α-SMA, Vimentin, Laminin, FAS, FAS-L, Fc, ICAM-1, ICAM-2, ICAM-3, ICAM-4, ICAM-5, PECAM-1, MICA, MICB, UL16, ULBP1, ULBP2, ILBP3, ULBP4, ULBP5, ULBP6, MULT1, RAE1α, β, γ, δ, and ε, A₁R, A₂AR, A_{2B}R, and A₃R, H60a, H60b, and H60c. In various embodiments, Integrins are selected from the group consisting of α1, α2, αIIb, α3, α4, α5, α6, α7, α8, α9, α10, α11, αD, αE, αL, αM, αV, αX, β1, β2, β3, β4, β5, β6, β7, β8, or combinations thereof. In various embodiments, TCR is selected from the group consisting of α, β, γ, δ, ε, and ζ TCR. Several methods have been described in the literature for assaying of cell-surface protein expression, including Flow Cytometry and Mass Cytometry (CyTOF). The presence or abundance of one or more of these cell-surface proteins indicates that the patient is amendable to treatment with the methods disclosed herein.

In various embodiments, the analysis of cells from one or more blood samples collected from a subject having one or more tumors characterized as immune refractory, immunologically protected, or immunologically “cold” demonstrates a high neutrophil to lymphocyte ratio (NLR). In various embodiments, the analysis of cells from one or more blood samples collected from a subject having one or more tumors characterized as immune refractory, immunologically protected, or immunologically “cold” demonstrates an NLR≥2. In various embodiments, the analysis of cells from one or more blood samples collected from a subject having one or more tumors characterized as immune refractory, immunologically protected, or immunologically “cold” demonstrates NLR of between 2 and 10 (e.g., NLR of 2, 3, 4, 5, 6, 7, 8, 9, and 10, inclusive of all values and ranges between these values). In various embodiments, NLR is used to determine the prognosis for a subject suffering from cancer and having one or more tumors characterized as immune refractory, immunologically protected, or immunologically “cold.” In various embodiments, NLR≥2 determines a poor prognosis.

In various embodiments, the cells analyzed from one or more blood samples collected from a subject suffering from cancer are circulating tumor cells (CTCs). In various embodiments, the assay of one or more blood samples collected from a subject suffering from cancer demonstrates increased frequency of CTCs compared to the analysis of one or more blood samples collected from one or more healthy subjects or one or more subjects suffering from cancer and responsive to treatment. In various embodiments, the frequency of circulating tumor cells in one or more blood samples of a subject having one or more tumors characterized as immune refractory, immunologically protected, or immunologically “cold” is ≥3 or ≤5 CTCs per 7.5 ml blood.

In various embodiments, the tumor characteristic of a subject suffering from cancer is determined from the analysis of proteins in one or more blood samples of the subject. In various embodiments, the tumor characteristic of a subject suffering from cancer is determined by comparing the analysis of proteins in one or more blood samples from the subject suffering from cancer to the analysis of one or more blood samples from one or more healthy subjects or one or more subjects suffering from cancer and responsive to treatment. In various embodiments, the protein is an intracellular protein or a secreted protein. In various embodiments, the protein is selected from the group consisting of cytokines, chemokines, growth factors, enzymes, proteases, and nucleases. In various embodiments, cytokines and chemokines are selected from the group consisting of IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-20, IL-21, IL-22, IL-23, IL-24, IL-25, IL-26, IL-27, IL-28, IL-29, IL-30, IL-31, IL-32, IL-33, IL-35, IL-36, CCL1, CCL2, CCL3, CCL4, CCL5, CCL6, CCL7, CCL8, CCL9, CCL10, CCL11, CCL12, CCL14, CCL15, CCL16, CCL17, CCL18, CCL19, CCL20, CCL21, CCL22, CCL23, CCL24, CCL25, CCL26, CCL27, CCL28, CXCL1, CXCL2 (MCP-1), CXCL3 (MIP-1α), CXCL4 (MIP-1β), CXCL5 (RANTES), CXCL6, CXCL7, CXCL8, CXCL9, CXCL10, CXCL11, CXCL12, CXCL13, CXCL14, CXCL15, CXCL16, CXCL17, IFN-α, IFN-β, IFN-γ, Granzyme-B, Perforin, TNF-α, TGF-β1, TGF-β2, and TGF-β3. In various embodiments, the growth factors are selected from the group consisting of EGF, FGF, NGF, PDGF, VEGF, IGF, GMCSF, GCSF, TGF, Erythropoietin, TPO, BMP, HGF, GDF, Neurotrophins, MSF, SGF, GDF, G-CSF, and GM-CSF. In various embodiments, the protein is a protease is selected from the group consisting of aspartic protease, a cysteine protease, a metalloprotease, a serine protease, or a threonine protease. In some embodiments, the protein is a protease is selected from the group consisting of ADAM1, ADAM2, ADAM7, ADAM8, ADAM9, ADAM10, ADAM11, ADAM12, ADAM15, ADAM17, ADAM18, ADAM19, ADAM20, ADAM21, ADAM22, ADAM23, ADAM28, ADAM29, ADAM30, ADAM33, MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27 and MMP28. In various embodiments, the protein is an enzyme selected from the group consisting of arginase, asparaginase, kynurinase, indoleamine 2,3 dioxygenase (IDO1 and IDO2), tryptophan 2,3 dioxygenase (TDO), and IL411. In various embodiments, the protein is associated with apoptosis. In various embodiments, proteins associated with apoptosis are selected from the group consisting of P53, Caspase 1, Caspase 2, Caspase 3, Caspase 4, Caspase 5, Caspase 6, Caspase 7, Caspase 8, Caspase 9, Caspase 10, Caspase 11, Caspase 12, Caspase 13, Caspase 14, BCL-2, BCL-XL, MCL-1, CED-9, A1, BFL1, BAX, BAK, DIVA, BCL-XS, BIK, BIM, BAD, BID, and EGL-1. Several methods have been described in the literature for assaying proteins from blood samples, including western blot, and ELISA.

In various embodiments, the analysis of proteins from one or more blood samples of a subject having one or more tumors that are characterized as immune refractory, immunologically protected, or immunologically “cold” demonstrates increased levels of tumor promoting, anti-inflammatory, or immune suppressive proteins. In various embodiments, the tumor promoting, anti-inflammatory, or immune suppressive proteins are cell-surface proteins, intracellular proteins, or secreted proteins. In various embodiments, the tumor promoting, anti-inflammatory, or immune suppressive proteins are selected from the group consisting of CD39, CD79, MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, MMP28, CXCL12, GM-CSF, G-CSF, TGF-β1, TGF-β2, TGF-β, arginase, asparaginase, kyneurinase, indoleamine 2,3 dioxygenase (IDO1 and IDO2), tryptophan 2,3 dioxygenase (TDO), myeloperoxidase (MPO), neutrophil elastase (NE), and IL411. In various embodiments, the levels of tumor promoting, anti-inflammatory, or immune suppressive proteins in one or more blood samples of a subject having one or more tumors that are characterized as immune refractory, immunologically protected, or immunologically “cold” are increased by 5-100% (e.g., increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more blood samples collected from one or more healthy subjects or one or more subjects suffering from cancer and responsive to treatment. In various embodiments, the levels of tumor promoting, anti-inflammatory, or immune suppressive proteins in one or more blood samples of a subject having one or more tumors that are characterized as immune refractory, immunologically protected, or immunologically “cold” are increased by 2-100 fold (e.g., about 2, 5, 10, 15, 20, 25, 30, 35, 40, 45, 50, 55, 60, 65, 70, 75, 80, 85, 90, 95, or 100 fold, inclusive of all values and ranges between these values) compared to one or more blood samples collected from one or more healthy subjects or one or more subjects suffering from cancer and responsive to treatment. In various embodiments, the analysis of proteins from one or more blood samples of a subject having one or more tumors that are characterized as immune refractory, immunologically protected, or immunologically “cold” demonstrate reduced levels, low levels, or absence of tumor inhibiting, anti-tumor, or pro-inflammatory proteins. In various embodiments, tumor inhibiting, anti-tumor, or pro-inflammatory proteins are selected from the group consisting of IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-20, IL-21, IL-22, IL-23, IL-24, IL-25, IL-26, IL-27, IL-28, IL-29, IL-30, IL-31, IL-32, IL-33, IL-35, IL-36, cell-surface IL-15, CXCL2 (MCP-1), CXCL3 (MIP-1α), CXCL4 (MIP-1β), CXCL5 (RANTES), IFN-α, IFN-β, IFN-γ, Granzyme-B, Perforin, and TNF-α. In various embodiments, the levels of tumor inhibiting, anti-tumor, or pro-inflammatory proteins in one or more blood samples of a subject having one or more tumors that are characterized as immune refractory, immunologically protected, or immunologically “cold” are decreased by 5-100% (e.g., reduced relative to a healthy subject or a subject suffering from cancer and responsive to treatment by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more blood samples collected from one or more healthy subjects or one or more subjects suffering from cancer and responsive to treatment. In various embodiments, the levels of tumor inhibiting, anti-tumor, or pro-inflammatory proteins in one or more blood samples of a subject having one or more tumors that are characterized as immune refractory, immunologically protected, or immunologically “cold” are decreased by 2-100 fold (e.g., about 2, 5, 10, 15, 20, 25, 30, 35, 40, 45, 50, 55, 60, 65, 70, 75, 80, 85, 90, 95, or 100 fold, inclusive of all values and ranges between these values) compared to one or more blood samples collected from one or more healthy subjects or subjects suffering from cancer and responsive to treatment. Several methods have been described in the literature for assaying proteins from blood samples, including western blot, and ELISA.

In various embodiments, the analysis of one or more blood samples of a subject having one or more tumors that are characterized as immune refractory, immunologically protected, or immunologically “cold” demonstrates increased levels of neutrophil extracellular traps (NETs). In various embodiments, the analysis of one or more blood samples of a subject having one or more tumors that are characterized as immune refractory, immunologically protected, or immunologically “cold” demonstrates increased levels of neutrophil extracellular traps (NETs) compared to the analysis of one or more blood samples from one or more healthy subjects or one or more subjects suffering from cancer and responsive to treatment. In various embodiments, the levels of NETs in one or more blood samples of a subject having one or more tumors that are characterized as immune refractory, immunologically protected, or immunologically “cold” are increased by 5-100% (e.g., increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more blood samples collected from one or more healthy subjects or one or more subjects suffering from cancer and responsive to treatment. In various embodiments, the levels of NETs in one or more blood samples of a subject having one or more tumors that are characterized as immune refractory, immunologically protected, or immunologically “cold” are increased by 2-100 fold (e.g., about 2, 5, 10, 15, 20, 25, 30, 35, 40, 45, 50, 55, 60, 65, 70, 75, 80, 85, 90, 95, or 100 fold, inclusive of all values and ranges between these values) compared to one or more blood samples collected from one or more healthy subjects or one or more subjects suffering from cancer and responsive to treatment. Several methods have been described in the literature for assaying NETs from blood samples, including western blot, ELISA, and flow cytometry.

In various embodiments, the tumor characteristic of a subject suffering from cancer is determined from the analysis of nucleic acids in one or more blood samples of the subject. In various embodiments, the tumor characteristic of a subject suffering from cancer is determined by comparing the analysis of nucleic acids in one or more blood samples from the subject suffering from cancer to the analysis of one or more blood samples from one or more healthy subjects or one or more subjects suffering from cancer and responsive to treatment. In various embodiments, the nucleic acid is selected from the group comprising DNA, ssDNA, circulating tumor DNA (ctDNA), RNA, mRNA, dsRNA, siRNA, miRNA, and lncRNA. In various embodiments, the analysis of ctDNA from one or more blood samples of a subject having one or more tumors that are characterized as immune refractory, immunologically protected, or immunologically “cold” demonstrates low levels or absence of one or more tumor mutations, tumor antigens, or neoantigens. In various embodiments, the analysis of ctDNA from one or more blood samples of a subject suffering from cancer demonstrates a low or no tumor mutation burden. In various embodiments, the analysis of ctDNA from one or more blood samples of a subject having one or more tumors that are characterized as immune refractory, immunologically protected, or immunologically “cold” demonstrates a tumor mutation burden of between 5 and 0.001 somatic mutations per mega base pairs (e.g., about 5, about 4, about 3, about 2, about 1, about 0.9, about 0.8, about 0.7, about 0.6, about 0.5, about 0.4, about 0.3, about 0.2, about 0.1, about 0.09, about 0.08, about 0.07, about 0.06, about 0.05, about 0.04, about 0.03, about 0.02, about 0.01, about 0.009, about 0.008, about 0.007, about 0.006, about 0.005, about 0.004, about 0.003, about 0.002, or 0.001, inclusive of all values and ranges between these values). In various embodiments, the nucleic acid analysis is performed by PCR, RT-PCR, qRT-PCR, next-generation sequencing (NGS), RNA-seq, ATAC-seq, exome sequencing, Southern Blot, microarray analysis, or single-cell sequencing.

In various embodiments, the tumor characteristic of a subject is determined from the gene expression analysis from nucleic acids in one or more blood samples of a subject suffering from cancer. In various embodiments, gene expression analysis from nucleic acids in one or more blood samples of a subject having one or more tumors that are characterized as immune refractory, immunologically protected, or immunologically “cold” demonstrates increased expression of tumor promoting, tumor permissive, or immune suppressive genes compared to the analysis of one or more blood samples from one or more healthy subjects or one or more subjects suffering from cancer and responsive to treatment. In various embodiments, the expression of tumor promoting, tumor permissive, or immune suppressive genes in one or more blood samples of a subject having one or more tumors that are characterized as immune refractory, immunologically protected, or immunologically “cold” is increased by 5-100% (e.g., increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% compared to one or more blood samples collected from one or more healthy subjects or one or more subjects suffering from cancer and responsive to treatment. In various embodiments, the expression of tumor promoting, tumor permissive, or immune suppressive genes in one or more blood samples of a subject having one or more tumors that are characterized as immune refractory, immunologically protected, or immunologically “cold” is increased by 2-100 fold (e.g., about 2, 5, 10, 15, 20, 25, 30, 35, 40, 45, 50, 55, 60, 65, 70, 75, 80, 85, 90, 95, or 100 fold, inclusive of all values and ranges between these values) compared to one or more blood samples collected from one or more healthy subjects or one or more subjects suffering from cancer and responsive to treatment. In various embodiments, the analysis of nucleic acids in one or more blood samples of a subject having one or more tumors that are characterized as immune refractory, immunologically protected, or immunologically “cold” demonstrates decreased expression of tumor inhibiting, anti-tumor, or pro-inflammatory genes compared to the analysis of one or more blood samples from one or more healthy subjects or one or more subjects suffering from cancer and responsive to treatment. In various embodiments, the analysis of nucleic acids in one or more blood samples of a subject having one or more tumors that are characterized as immune refractory, immunologically protected, or immunologically “cold” demonstrates low or no expression of tumor inhibiting, anti-tumor, or anti-inflammatory genes compared to the analysis of one or more blood samples from one or more healthy subjects or one or more subjects suffering from cancer and responsive to treatment. In various embodiments, the expression of tumor inhibiting, anti-tumor, or pro-inflammatory genes in one or more blood samples of a subject having one or more tumors that are characterized as immune refractory, immunologically protected, or immunologically “cold” is decreased by 5-100% (e.g., decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% compared to a healthy subject or a subject suffering from cancer who is responsive to therapy. In various embodiments, the expression of tumor inhibiting, anti-tumor, or pro-inflammatory genes in one or more blood samples of a subject having one or more tumors that are characterized as immune refractory, immunologically protected, or immunologically “cold” is reduced by 2-100 fold (e.g., reduced relative to a healthy subject or a subject suffering from cancer and responsive to therapy by about 2, 5, 10, 15, 20, 25, 30, 35, 40, 45, 50, 55, 60, 65, 70, 75, 80, 85, 90, 95, or 100 fold, inclusive of all values and ranges between these values). In various embodiments, the gene expression analysis is performed by PCR, RT-PCR, qRT-PCR, next-generation sequencing (NGS), RNA-seq, ATAC-seq, exome sequencing, Southern Blot, microarray analysis, or single-cell sequencing.

In various embodiments, the tumor characteristic of a subject suffering from cancer is determined from the analysis of one or more tumor samples collected from the subject. In various embodiments, the tumor sample is a biopsy. In various embodiments, the tumor characteristic of a subject suffering from cancer is determined from the analysis of cells, proteins, or nucleic acids in one or more tumor samples collected from the subject. In various embodiments, the tumor characteristic of a subject suffering from cancer is determined by comparing the analysis of cells, proteins, or nucleic acids in one or more tumor samples from the subject suffering from cancer to the analysis of tissue samples from one or more healthy subjects or one or more subjects suffering from cancer and responsive to treatment. In various embodiments, the cells analyzed in one or more tumor samples are leukocytes, epithelial cells, mesenchymal cells, mesenchymal stem cells, stromal cells, endothelial cells, fibroblasts, pericytes, adipocytes, and cancer stem cells. In various embodiments, the leukocytes are myeloid cells and lymphoid cells. In various embodiments, myeloid cells are monocytes, macrophages, neutrophils, granulocytes, dendritic cells, mast cells, eosinophils, and basophils. In various embodiments, the lymphoid cells are T cells, B cells, NK cells, NK-T cells, or iNK cells.

In various embodiments, the analysis of cells from one or more tumor samples collected from a subject having one or more tumors that are characterized as immune refractory, immunologically protected, or immunologically “cold” demonstrates the presence of immune suppressive cells. In various embodiments, the analysis of one or more tumor samples collected from a subject having one or more tumors that are characterized as immune refractory, immunologically protected, or immunologically “cold” demonstrates the presence of immune suppressive cells in the tumor core. In various embodiments, the analysis of cells from one or more tumor samples collected from a subject having one or more tumors that are characterized as immune refractory, immunologically protected, or immunologically “cold” demonstrates increased levels of immune suppressive cells. In various embodiments, the analysis of one or more tumor samples demonstrates increased levels of immune suppressive cells in the tumor core. In various embodiments, the immune suppressive cells are myeloid derived suppressor cells (MDSCs), tumor-associated macrophages (TAMs), neutrophils, T_{reg} cells, and B_{reg} cells. In various embodiments, MDSCs are monocytic MDSCs (M-MDSCs) and polymorphonuclear MDSCs (PMN-MDSCs). In various embodiments, the TAMs are M2 TAMs. In various embodiments, the immune suppressive cells are CAFs. In various embodiments, the levels of immune suppressive cells in one or more tumor samples of a subject having one or more tumors that are characterized as immune refractory, immunologically protected, or immunologically “cold” are increased by 5-100% (e.g., increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% compared to tissue samples of one or more healthy subjects or one or more subjects suffering from cancer and responsive to treatment. In various embodiments, the levels of immune suppressive cells in one or more tumor samples of a subject having one or more tumors that are characterized as immune refractory, immunologically protected, or immunologically “cold” are increased by 2-100-fold (e.g., increased relative to a healthy subject or a subject suffering from cancer and responsive to treatment by about 2, 5, 10, 15, 20, 25, 30, 35, 40, 45, 50, 55, 60, 65, 70, 75, 80, 85, 90, 95, or 100 fold, inclusive of all values and ranges between these values) compared to one or more tissue samples or one or more healthy subjects or one or more subjects suffering from cancer and responsive to treatment.

In various embodiments, the analysis of cells from one or more tumor samples collected from a subject having one or more tumors that are characterized as immune refractory, immunologically protected, or immunologically “cold” demonstrates the absence of leukocytes. In various embodiments, the analysis of cells from one or more tumor samples collected from a subject having one or more tumors that are characterized as immune refractory, immunologically protected, or immunologically “cold” demonstrates the reduced or low levels of leukocytes. In various embodiments, the frequency of leukocytes is ≤50%, ≤40%, ≤30%, ≤20%, ≤10%, or ≤5%, inclusive of all values and ranges between these values, of all cells analyzed.

In various embodiments, the analysis of cells from one or more tumor samples collected from a subject having one or more tumors that are characterized as immune refractory, immunologically protected, or immunologically “cold” demonstrates the absence of activated pro-inflammatory immune cells. In various embodiments, the analysis of cells from one or more tumor samples collected from a subject having one or more tumors that are characterized as immune refractory, immunologically protected, or immunologically “cold” demonstrates the absence of activated pro-inflammatory immune cells from the tumor core. In various embodiments, the analysis of cells from one or more tumor samples collected from a subject having one or more tumors that are characterized as immune refractory, immunologically protected, or immunologically “cold” demonstrates low or reduced levels of activated pro-inflammatory immune cells. In various embodiments, the analysis of cells from one or more tumor samples collected from a subject having one or more tumors that are characterized as immune refractory, immunologically protected, or immunologically “cold” demonstrates low or reduced levels of activated pro-inflammatory immune cells in the tumor core. In various embodiments, the activated pro-inflammatory cells are dendritic cells (DCs), macrophages, M1 macrophages, T-cells, B-cells, NK cells, NK-T cells, and iNK cells. In various embodiments, the frequency of pro-inflammatory immune cells is ≤50%, ≤40%, ≤30%, ≤20%, ≤10%, or ≤5%, inclusive of all values and ranges between these values, of all cells analyzed.

In various embodiments, the tumor characteristic of a subject suffering from cancer is determined from the analysis of the location of immune cells in one or more tumor samples of the subject. In various embodiments, immune cells in one or more tumor samples of a subject having one or more immune refractory, immunologically protected, or immunologically “cold” are located in the tumor periphery. In various embodiments, immune cells in one or more tumor samples of a subject having one or more immune refractory, immunologically protected, or immunologically “cold” are absent from the tumor core. In various embodiments, immune cells in one or more tumor samples of a subject having one or more immune refractory, immunologically protected, or immunologically “cold” are reduced in the tumor core. In various embodiments, immune cells in the tumor core are reduced by 5-100% (e.g., relative to a healthy subject or a subject suffering from cancer and responsive to treatment by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% compared to one or more samples from one or more healthy subjects or one or more subjects suffering from cancer and responsive to treatment.

In various embodiments, the tumor characteristic of a subject suffering from cancer is determined from the analysis of the location of stromal cells in one or more tumor samples of the subject. In various embodiments, the stromal cells are CAFs, pericytes, adipocytes, and endothelial cells. In various embodiments, CAFs in one or more tumor samples of a subject having one or more immune refractory, immunologically protected, or immunologically “cold” tumors are increased in the tumor periphery. In various embodiments, CAFs in one or more tumor samples of a subject having one or more immune refractory, immunologically protected, or immunologically “cold” tumors are increased in the tumor core. In various embodiments, the frequency of CAFs in the tumor periphery is increased by 5-100% (e.g., increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more tissue sample from one or more healthy subjects or subjects suffering from cancer and responsive to treatment. In various embodiments, the frequency of CAFs in the tumor periphery is increased by 2-100 fold (e.g., increased by about 2, 5, 10, 15, 20, 25, 30, 35, 40, 45, 50, 55, 60, 65, 70, 75, 80, 85, 90, 95 fold, inclusive of all values and ranges between these values) compared to one or more tissue samples from one or more healthy subjects or one or more subjects suffering from cancer and responsive to treatment. In various embodiments, the frequency of CAFs in the tumor core is increased by 5-100% (e.g., increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more tissue samples of one or more healthy subjects or one or more subjects suffering from cancer and responsive to treatment. In various embodiments, the frequency of CAFs in the tumor core is increased by 2-100 fold (e.g., increased relative to a healthy subject or a subject suffering from cancer and responsive to treatment by about 2, 5, 10, 15, 20, 25, 30, 35, 40, 45, 50, 55, 60, 65, 70, 75, 80, 85, 90, 95 fold, inclusive of all values and ranges between these values) compared to one or more healthy tissue samples.

In various embodiments, the analysis of cells in one or more tumor samples of a subject suffering from cancer is performed by the assay of cell-surface proteins. In various embodiments, the cell-surface proteins are selected from the group consisting of receptor tyrosine kinase (RTK), CD1c, CD2, CD3, CD4, CD5, CD8, CD9, CD10, CD11b, CD11c, CD14, CD15, CD16, CD18, CD19, CD20, CD21, CD22, CD23, CD24, TACI, CD25, CD27, CD28, CD30, CD30L, CD31, CD32, CD32b, CD34, CD33, CD38, CD39, CD40, CD40-L, CD41b, CD42a, CD42b, CD43, CD44, CD45, CD47, CD45RA, CD45RO, CD48, CD52, CD55, CD56, CD58, CD61, CD66b, CD70, CD72, CD79, CD68, CD84, CD86, CD93, CD94, CD95, CRACC, BLAME, BCMA, CD103, CD107, CD112, CD120a, CD120b, CD123, CD125, CD134, CD135, CD140a, CD141, CD154, CD155, CD160, CD163, CD172a, XCR1, CD203c, CD204, CD206, CD207 CD226, CD244, CD267, CD268, CD269, CD355, CD358, NKG2A, NKG2B, NKG2C, NKG2D, NKG2E, NKG2F, NKG2H, KIR2DL1, KIR2DL2, KIR2DL3, KIR2DL5A, KIR2DL5B, KIR3DL1, KIR3DL2, KIR3DL3, KIR3DL4, KIR2DS1, KIR2DS2, KIR2DS3, KIR2DS4, KIR2DS5, DAP12, KIR3DS, NKp44, NKp46, TCR, BCR, Integrins, FcβεRI, MHC-1, MHC-II, IL-1R, IL-2Rα, IL-2Rβ, IL-2Rγ, IL-3Rα, CSF2RB, IL-4R, IL-SRα, IL-6Rα, gp130, IL-7Rα, IL-9R, IL-12Rβ1, IL-12Rβ2, IL-13Rα1, IL-13Rα2, IL-15Rα, IL-21R, IL-23R, IL-27Rα, IL-31Rα, OSMR, CSF-1R, cell-surface IL-15, IL-10Rα, IL-10Rβ, IL-20Rα, IL-20Rβ, IL-22Rα1, IL-22Rα2, IL-22Rβ, IL-28RA, PD-1, PD-1H, BTLA, CTLA-4, PD-L1, PD-L2, 2B4, B7-1, B7-2, B7-H1, B7-H4, B7-DC, DR3, LIGHT, LAIR, LTα1β2, LTβR, TIM-1, TIM-3, TIM-4, TIGIT, LAG-3, ICOS, ICOS-L, SLAM, SLAMF2, OX-40, OX-40L, GITR, GITRL, TL1A, HVEM, 41-BB, 41BB-L, TL-1A, TRAF1, TRAF2, TRAF3, TRAF5, BAFF, BAFF-R, APRIL, TRAIL, RANK, AITR, TRAMP, CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6, CXCR7, CLECL9a, DC-SIGN, IGSF4A, SIGLEC, EGFR, PDGFR, VEGFR, FAP, α-SMA, Vimentin, Laminin, FAS, FAS-L, F_{c}, ICAM-1, ICAM-2, ICAM-3, ICAM-4, ICAM-5, PECAM-1, MICA, MICB, UL16, ULBP1, ULBP2, ILBP3, ULBP4, ULBP5, ULBP6, MULT1, RAE1α, β, γ, δ, and ε, A₁R, A_{2A}R, A_{2B}R, and A₃R, H60a, H60b, and H60c. In various embodiments, Integrins are selected from the group consisting of α1, α2, αIIb, α3, α4, α5, α6, α7, α8, α9, α10, α11, αD, αE, αL, αM, αV, αX, β1, β2, β3, β4, β5, β6, β7, β8, or combinations thereof. In various embodiments, TCR is selected from the group consisting of α, β, γ, δ, ε, and ζ TCR. Several methods have been described in the literature for assaying of cell-surface protein expression from tumor samples, including immunohistochemistry, immunofluorescence, western blot, flow cytometry, and Mass Cytometry (CyTOF).

Tumor core is generally described as the densely packed, central, bulk-forming and differentiated region of the tumor. In contrast, the tumor periphery is generally described as the invasive edge of the tumor that interacts with the surrounding stroma and parenchyma.

In various embodiments, the tumor characteristic of a subject suffering from cancer is determined from the analysis of proteins in one or more tumor samples of the subject. In various embodiments, the tumor characteristic of a subject suffering from cancer is determined by comparing the analysis of proteins in one or more tumor samples from the subject suffering from cancer to the analysis of one or more tissues from one or more healthy subjects or one or more subjects suffering from cancer and responsive to treatment. In various embodiments, the protein is intracellular or extracellular. In various embodiments, the protein is selected from the group consisting of cytokines, chemokines, growth factors, enzymes, proteases, and nucleases. In various embodiments, cytokines and chemokines are selected from the group consisting of IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-20, IL-21, IL-22, IL-23, IL- 24, IL-25, IL-26, IL-27, IL-28, IL-29, IL-30, IL-31, IL-32, IL-33, IL-35, IL-36, CCL1, CCL2, CCL3, CCL4, CCL5, CCL6, CCL7, CCL8, CCL9, CCL10, CCL11, CCL12, CCL14, CCL15, CCL16, CCL17, CCL18, CCL19, CCL20, CCL21, CCL22, CCL23, CCL24, CCL25, CCL26, CCL27, CCL28, CXCL1, CXCL2 (MCP-1), CXCL3 (MIP-1α), CXCL4 (MIP-1β), CXCL5 (RANTES), CXCL6, CXCL7, CXCL8, CXCL9, CXCL10, CXCL11, CXCL12, CXCL13, CXCL14, CXCL15, CXCL16, CXCL17, IFN-α, IFN-β, IFN-γ, Granzyme-B, Perforin, TNF-α, TGF-β1, TGF-β2, and TGF-β3. In various embodiments, the growth factors are selected from the group consisting of EGF, FGF, NGF, PDGF, VEGF, IGF, GMCSF, GCSF, TGF, Erythropoietin, TPO, BMP, HGF, GDF, Neurotrophins, MSF, SGF, GDF, G-CSF, and GM-CSF. In various embodiments, the protein is a protease is selected from the group consisting of aspartic protease, a cysteine protease, a metalloprotease, a serine protease, or a threonine protease. In some embodiments, the protein is a protease is selected from the group consisting of ADAM1, ADAM2, ADAM7, ADAM8, ADAM9, ADAM10, ADAM11, ADAM12, ADAM15, ADAM17, ADAM18, ADAM19, ADAM20, ADAM21, ADAM22, ADAM23, ADAM28, ADAM29, ADAM30, ADAM33, MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, and MMP28. In various embodiments, the protein is an enzyme selected from the group consisting of arginase, asparaginase, kynurinase, indoleamine 2,3 dioxygenase (IDO1 and IDO2), tryptophan 2,3 dioxygenase (TDO), and IL411. In various embodiments, the protein is associated with apoptosis. In various embodiments, proteins associated with apoptosis are selected from the group consisting of P53, Caspase 1, Caspase 2, Caspase 3, Caspase 4, Caspase 5, Caspase 6, Caspase 7, Caspase 8, Caspase 9, Caspase 10, Caspase 11, Caspase 12, Caspase 13, Caspase 14, BCL-2, BCL-XL, MCL-1, CED-9, A1, BFL1, BAX, BAK, DIVA, BCL-XS, BIK, BIM, BAD, BID, and EGL-1. Several methods have been described in the literature for assaying proteins from tumor samples, including immunohistochemistry, immunofluorescence, western blot, and ELISA.

In various embodiments, the analysis of proteins from one or more tumor samples of a subject having one or more tumors characterized as immune refractory, immunologically protected, or immunologically “cold” demonstrates increased levels of proteins associated with tumor progression, anti-inflammatory activity, or immune suppression. In various embodiments, proteins associated with tumor progression, anti-inflammatory activity, or immune suppression are cell-surface proteins, intracellular proteins, or secreted proteins. In various embodiments, proteins associated with tumor progression, anti-inflammatory activity, or immune suppression are selected from the group consisting of CD39, CD47, CD79, CD140a, CD163, CD206, FOXP3, FAP, PD-1, PD-L1, PD-L2, CSF-1R, A₁R, A_{2A}R, A_{2B}R, A3R, TIM-1, TIM-3, TIM-4, TIGIT, CSFR, SIGLEC, MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, MMP28, CXCL12, GM-CSF, G-CSF, FAP, TGF-β1, TGF-β2, TGF-β3, arginase, asparaginase, kyneurinase, indoleamine 2,3 dioxygenase (IDO1 and IDO2), tryptophan 2,3 dioxygenase (TDO), myeloperoxidase (MPO), neutrophil elastase (NE), and IL411. In various embodiments, the levels of proteins associated with tumor progression, anti-inflammatory activity, or immune suppression in one or more tumor samples of a subject having one or more tumors characterized as immune refractory, immunologically protected, or immunologically “cold” are increased by 5-100% (e.g., increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more tissue samples from one or more healthy subjects or one or more subjects suffering from cancer and responsive to treatment. In various embodiments, the levels of proteins associated with tumor progression, anti-inflammatory activity, or immune suppression in one or more tumor samples of a subject having one or more tumors characterized as immune refractory, immunologically protected, or immunologically “cold” are increased by 2-100 fold (e.g., increased relative to a healthy subject or a subject suffering from cancer and responsive to treatment by about 2, 5, 10, 15, 20, 25, 30, 35, 40, 45, 50, 55, 60, 65, 70, 75, 80, 85, 90, 95 fold, inclusive of all values and ranges between these values) compared to one or more tissue samples from one or more healthy subjects or subjects suffering from cancer and responsive to treatment.

In various embodiments, the analysis of proteins from one or more tumor samples of a subject having one or more tumors characterized as immune refractory, immunologically protected, or immunologically “cold” demonstrate reduced levels, low levels, or absence of proteins associated with tumor growth inhibition, anti-tumor activity, or pro-inflammatory activity. In various embodiments, proteins associated with tumor growth inhibition, anti-tumor activity, or pro-inflammatory activity are selected from the group consisting of CD44, CD56, CD103c, CD69, KG2A, NKG2B, NKG2C, NKG2D, NKG2E, NKG2F, NKG2H, ICOS, ICOS-L, SLAM, SLAMF2, OX-40, OX-40L, GITR, GITRL, TL-1A, HVEM, 41-BB, 41BB-L, TRAF1, TRAF2, TRAF3, TRAF5, BAFF, BAFF-R, APRIL, TRAIL, RANK, AITR, TRAMP, IL-1α, IL-1β, IL-2, IL-3, IL-4, IL-5, IL-6, IL-7, IL-8, IL-9, IL-10, IL-11, IL-12, IL-13, IL- 14, IL-15, IL-16, IL-17, IL-18, IL-20, IL-21, IL-22, IL-23, IL-24, IL-25, IL-26, IL-27, IL-28, IL-29, IL-30, IL-31, IL-32, IL-33, IL-35, IL-36, CXCL2 (MCP-1), CXCL3 (MIP-1α), CXCL4 (MIP-1β), CXCL5 (RANTES), IFN-α, IFN-β, IFN-γ, Granzyme-B, Perforin, and TNF-α. In various embodiments, the levels of proteins associated with tumor growth inhibition, anti-tumor activity, or pro-inflammatory activity are reduced by 5-100% (e.g., reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more samples collected from one or more healthy tissues or one or more tumor samples collected from a subject suffering from cancer and responsive to treatment. Several methods have been described for assaying proteins from tumor samples, including immunohistochemistry, immunofluorescence, western blot, intracellular flow cytometry, and ELISA.

In various embodiments, the tumor characteristic of a subject suffering from cancer is determined from the Tumor Proportion Score (TPS) for PD-L1 expression in one or more tumor samples from the subject. In various embodiments, the TPS of a subject having one or more tumors that are characterized as immune refractory, immunologically protected, or immunologically “cold” of between 1 and 50 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, inclusive of all ranges between these values). In various embodiments, the TPS of a subject having one or more tumors that are characterized as immune refractory, immunologically protected, or immunologically “cold” is ≤1. TPS for PD-L1 expression is defined as the percentage of viable tumor cells demonstrating partial or complete membrane staining by immunohistochemical analysis.

In various embodiments, the tumor characteristic of a subject suffering from cancer is determined from the Combined Positivity Score (CPS) for PD-L1 expression in one or more tumor samples from the subject. In various embodiments, the CPS of a subject having one or more tumors that are characterized as immune refractory, immunologically protected, or immunologically “cold” is ≤10 (e.g., 1, 2, 3, 4, 5, 6, 7, 8, 9, or 10, inclusive of all ranges between these values). In various embodiments, the CPS is ≤1. CPS for PD-L1 expression is determined from the immunohistochemical determination of the number of viable tumor cells, lymphocytes, and macrophages positive for PD-L1 as a percentage of all viable tumor cells.

In various embodiments, the tumor characteristic of a subject suffering from cancer is determined from microsatellite instability testing of one or more tumor samples from the subject. In various embodiments, the tumor characteristic of a subject suffering from cancer is determined by comparing microsatellite instability testing of one or more tumor samples to microsatellite stability testing from one or more healthy tissues of the subject. In various embodiments, the microsatellite instability testing is the assay of microsatellite markers. In various embodiments, the microsatellite instability testing is the assay of mismatch repair markers. In various embodiments, the microsatellite markers are selected from the group consisting of BAT25, BAT26, D2S123, D5S346, and D17S250. In various embodiments, the mismatch repair markers are selected from the group consisting of MLH1, MSH2, MLH6, and PMS2. In various embodiments, the subject has one or more immune refractory, immunologically protected, or immunologically “cold” tumors that are determined to be microsatellite instability low. In various embodiments, the subject has one or more immune refractory, immunologically protected, or immunologically “cold” tumors that are determined to be microsatellite stable. In various embodiments, the subject has one or more immune refractory, immunologically protected, or immunologically “cold” tumors that are mismatch repair proficient.

In various embodiments, the analysis of one or more tumor samples of a subject having one or more tumors characterized as immune refractory, immunologically protected, or immunologically “cold” demonstrates increased levels of neutrophil extracellular traps (NETs). In various embodiments, the analysis of one or more tumor samples of a subject having one or more tumors characterized as immune refractory, immunologically protected, or immunologically “cold” demonstrates increased levels of neutrophil extracellular traps (NETs) compared to the analysis of one or more tumor samples from one or more healthy subjects. In various embodiments, the levels of NETs in one or more tumor samples of a subject suffering from cancer are increased by 5-100% (e.g., increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more tissue samples from one or more healthy subjects or subject suffering from cancer and responsive to treatment. In various embodiments, the levels of NETs in one or more tumor samples of a subject suffering from cancer are increased by 2-100 fold (e.g., increased relative to a healthy subject or a subject suffering from cancer and responsive to treatment by about 2, 5, 10, 15, 20, 25, 30, 35, 40, 45, 50, 55, 60, 65, 70, 75, 80, 85, 90, 95 fold, inclusive of all values and ranges between these values) compared to one or more tissue samples from one or more healthy subjects or subjects suffering from cancer and responsive to treatment. Several methods have been described in the literature for assaying NETs, including western blot, ELISA, and flow cytometry.

In various embodiments, the tumor characteristic of a subject suffering from cancer is determined from the analysis of nucleic acids in one or more tumor samples of the subject. In various embodiments, the tumor characteristic of a subject suffering from cancer is determined by comparing the analysis of nucleic acids in one or more tumor samples from the subject suffering from cancer to the analysis of one or more tissue samples from one or more healthy subjects or subjects suffering from cancer and responsive to treatment. In various embodiments, the nucleic acid is selected from the group comprising DNA, ssDNA, RNA, mRNA, dsRNA, siRNA, miRNA, and lncRNA. In various embodiments, the nucleic acid analysis is performed by PCR, RT-PCR, qRT-PCR, next-generation sequencing (NGS), RNA-seq, ATAC-seq, exome sequencing, Southern Blot, microarray analysis, or single-cell sequencing.

In various embodiments, the analysis of nucleic acids from one or more tumor samples of a subject suffering from cancer is used to determine the tumor mutation burden. In various embodiments, the analysis of nucleic acids from one or more tumor samples of a subject having one or more tumors characterized as immune refractory, immunologically protected, or immunologically “cold” demonstrates a low tumor mutation burden. In various embodiments, the analysis of nucleic acids from one or more tumor samples a subject having one or more tumors characterized as immune refractory, immunologically protected, or immunologically “cold” demonstrates a tumor mutation burden of between 5 and 0.001 somatic mutations per mega base pairs (e.g., about 5, about 4, about 3, about 2, about 1, about 0.9, about 0.8, about 0.7, about 0.6, about 0.5, about 0.4, about 0.3, about 0.2, about 0.1, about 0.09, about 0.08, about 0.07, about 0.06, about 0.05, about 0.04, about 0.03, about 0.02, about 0.01, about 0.009, about 0.008, about 0.007, about 0.006, about 0.005, about 0.004, about 0.003, about 0.002, or 0.001, inclusive of all values and ranges between these values). In various embodiments, the nucleic acid analysis is performed by PCR, RT-PCR, qRT-PCR, next-generation sequencing (NGS), RNA-seq, ATAC-seq, exome sequencing, Southern Blot, microarray analysis, or single-cell sequencing.

In various embodiments, the analysis of nucleic acids in one or more tumor samples of a subject having one or more tumors characterized as immune refractory, immunologically protected, or immunologically “cold” demonstrates increased expression of genes associated with tumor promoting, tumor permissive, anti-inflammatory, or immune suppressive activity compared to the analysis of one or more tissue samples from one or more healthy subjects or subjects suffering from cancer and responsive to treatment. In various embodiments, genes associated with tumor promoting, tumor permissive, anti-inflammatory, or immune suppressive activity are selected from the group consisting of CD39, CD47, CD79, CD140a, CD163, CD206, FOXP3, FAP, PD-1, PD-L1, PD-L2, CSF-1R, A₁R, A_{2A}R, A_{2B}R, A₃R, TIM-1, TIM-3, TIM-4, TIGIT, CSFR, SIGLEC, MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, MMP28, CXCL12, GM-CSF, G-CSF, FAP, TGF-β1, TGF-β2, TGF-β3, arginase, asparaginase, kyneurinase, indoleamine 2,3 dioxygenase (IDO1 and IDO2), tryptophan 2,3 dioxygenase (TDO), myeloperoxidase (MPO), neutrophil elastase (NE), and IL411. In various embodiments, the expression of genes associated with tumor promoting, tumor permissive, anti-inflammatory, or immune suppressive activity is increased by 5-100% (e.g., increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more tissue samples of one or more healthy subjects or subjects suffering from cancer and responsive to treatment. In various embodiments, the gene expression analysis is performed by PCR, RT-PCR, qRT-PCR, next-generation sequencing (NGS), RNA-seq, ATAC-seq, exome sequencing, Southern Blot, microarray analysis, or single-cell sequencing.

In various embodiments, the analysis of nucleic acids in one or more tumor samples of a subject having one or more tumors characterized as immune refractory, immunologically protected, or immunologically “cold” demonstrates low or decreased expression of genes associated with tumor inhibiting, anti-tumor, or pro-inflammatory activity. In various embodiments, the analysis of nucleic acids in one or more tumor samples of a subject suffering from cancer demonstrates no expression of genes associated with tumor inhibiting, anti-tumor, or pro-inflammatory activity. In various embodiments, genes associated with tumor inhibiting, anti-tumor, or pro-inflammatory activity are selected from the group consisting of CD44, CD56, CD103c, CD69, KG2A, NKG2B, NKG2C, NKG2D, NKG2E, NKG2F, NKG2H, ICOS, ICOS-L, SLAM, SLAMF2, OX-40, OX-40L, GITR, GITRL, TL-1A, HVEM, 41-BB, 41BB-L, TRAF1, TRAF2, TRAF3, TRAF5, BAFF, BAFF-R, APRIL, TRAIL, RANK, AITR, TRAMP, cell-surface IL-15, IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-20, IL-21, IL-22, IL-23, IL-24, IL-25, IL-26, IL-27, IL-28, IL-29, IL-30, IL-31, IL-32, IL-33, IL-35, IL-36, CXCL2 (MCP-1), CXCL3 (MIP-1α), CXCL4 (MIP-1β), CXCL5 (RANTES), IFN-α, IFN-p, IFN-γ, Granzyme-B, Perforin, TNF-α, and p53. In various embodiments, the expression genes associated with tumor inhibiting, anti-tumor, or pro-inflammatory activity is decreased by 5-100% (e.g., decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50% compared to a healthy subject or a subject suffering from cancer responsive to treatment. In various embodiments, the gene expression analysis is performed by PCR, RT-PCR, qRT-PCR, next-generation sequencing (NGS), RNA-seq, ATAC-seq, exome sequencing, Southern Blot, microarray analysis, or single-cell sequencing.

In various embodiments, the disclosure provides a method of treating cancer in a subject comprising administering to the subject a RAS inhibitor or combination of compounds described herein, wherein the subject has one or more tumors that are resistant or unresponsive to treatment. In various embodiments, the subject has one or more tumors that are resistant or unresponsive to one or more treatments selected from the group consisting of surgery, radiation, chemotherapy, biologic agents, small molecules, cell-based therapy, hormone therapy, and immunotherapy. In various embodiments, treatment is a standard of care therapy, first-line therapy, second-line therapy, or third-line therapy. In various embodiments, the subject has one or more tumors that have progressed during one or more treatments, wherein the treatments are standard of care therapy, first-line therapy, second-line therapy, or third-line therapy.

First-line therapy is defined as a treatment that is administered to a subject suffering from cancer who has not received any prior treatment. Second-line therapy is defined as treatment that is administered to a subject suffering from cancer who has received prior first-line therapy but experienced disease progression during first-line treatment. Third-line therapy is defined as treatment that is administered to a subject suffering from cancer who has received prior first and second-line treatment but has experienced disease progression during second-line treatment. Each particular type of cancer has a first-line, second-line, and third-line therapy. The first-, second-, and third-line therapies for types of cancer are known in the art. In addition, FDA approved drug labels will indicate if a particular drug is approved as a first-, second-, or third-line therapy.

Several criteria and definitions published in the literature can be used to determine the effect of one or more treatments on tumors in a subject suffering from cancer. Based on these criteria, tumors are defined as “responsive,” “stable,” or “progressive” when they improve, remain the same, or worsen during treatment, respectively.

Examples of the commonly used criteria published in the literature include Response Evaluation Criteria in Solid Tumors (RECIST), Modified Response Evaluation Criteria in Solid Tumors (mRECIST), PET Response Criteria in Solid Tumors (PERCIST), Choi Criteria, Lugano Response Criteria, European Association for the Study of the Liver (EASL) Criteria, Response Evaluation Criteria in the Cancer of the Liver (RECICL), and WHO Criteria in Tumor Response.

In various embodiments, the disclosure provides a method of treating cancer in a subject comprising administering to the subject a RAS inhibitor or combination of compounds described herein, wherein the subject cannot tolerate standard of care therapy, first-line therapy, second-line therapy, or third-line therapy. In various embodiments, the disclosure provides a method of treating cancer in a subject comprising administering to the subject a RAS inhibitor or combination of compounds described herein, wherein the subject has experienced tumor recurrence after surgical resection of the primary tumor. In various embodiments, the disclosure provides a method of treating cancer in a subject comprising administering to the subject a RAS inhibitor or combination of compounds described herein, wherein the subject has a tumor that cannot be surgically removed. In various embodiments, the disclosure provides a method of treating cancer in a subject comprising administering to the subject a RAS inhibitor or combination of compounds described herein, wherein the subject has no treatment options available.

Several therapies used in the treatment of cancer (e.g., chemotherapies) are cytotoxic and are associated with significant side-effects and toxicities that are associated with poor outcomes and poor response to treatment. Prior to administering such treatments, clinicians rely on several assessment tools to help determine the risk of a subject suffering from cancer experiencing treatment related toxicities and adverse events. Based on the results of these assessments, a subject suffering from cancer is considered intolerant to therapy if they are determined to be at increased risk of experiencing therapy-related toxicities and adverse events resulting in poor outcomes. Examples of commonly used assessment tools used in the determination of therapy intolerance include Karnofsky Performance Status (KPS), Eastern Cooperative Oncology Group Performance Status (ECOG PS), Timed Get Up and Go (TUG), Short Physical Performance Battery (SPPB), Comprehensive Geriatric Assessment (CGA), Cancer Aging Research Group (CARG) Score, and Chemotherapy Risk Assessment Scale for High-Age Patients (CRASH).

### Treatment Outcomes and Clinical Endpoints

In various embodiments, the disclosure provides a method of treating lung cancer in a subject comprising administering to the subject a RAS inhibitor or combination of compounds described herein, wherein the subject has one or more immune refractory tumors. In various embodiments, the administering alters the tumor immune infiltrate. In various embodiments, the administering alters the anti-tumor immune response. In various embodiments, the administering alters the tumor microenvironment comprising tumor cells, immune cells, cancer stem cells, and stroma. In various embodiments, the administering transforms an immunologically cold tumor into an immunologically hot tumor. In various embodiments, the administering reduces tumor size or inhibits tumor growth. In various embodiments, the administering induces tumor cell death, apoptosis, or necrosis via direct particle uptake by tumor cells.

In various embodiments, the disclosure provides a method of treating cancer in a subject comprising administering to the subject a RAS inhibitor or combination of compounds described herein, wherein the subject has one or more tumors that are characterized as immunologically protected or immune refractory. In various embodiments, the administering alters the tumor-associated stroma comprising fibroblasts, cancer-associated fibroblasts, adipocytes, pericytes, endothelium, vasculature, lymphatic vessels, tumor-associated vasculature, mesenchymal stromal cells, mesenchymal stem cells, and extracellular matrix.

It is contemplated that the methods herein reduce tumor size or tumor burden in the subject, or reduce metastasis in the subject. In various embodiments, the methods reduce the tumor size by 10%, 20%, 30% or more. In various embodiments, the methods reduce tumor size by 10%, 15%, 20%, 25%, 30%, 35%, 40%, 45%, 50%, 55%, 60%, 65%, 70%, 75%, 80%, 85%, 90%, 95% or 100%, or including all values and ranges that lie in between these values.

Certain biomarkers may decrease in abundance when a tumor becomes immune refractory. It is contemplated herein that after treatment with a RAS inhibitor or combination of compounds described herein, the level of one or more of biomarkers increases by an amount in the range of from about 1.1 fold to about 10 fold, e.g., about 1.1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10 fold. Similarly, certain biomarkers increase in abundance when a tumor becomes immune refractory. After treatment with a RAS inhibitor or combination of compounds described herein, the level of one or more of such biomarkers decrease by an amount in the range of from about 1.1 fold to about 10 fold, e.g., about 1.1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10 fold. In various embodiments, administering a RAS inhibitor or combination of compounds described herein to a subject having one or more tumors characterized as immune refractory, immunologically protected, or immunologically “cold” reduces the levels of immune suppressive cells in blood. In various embodiments, the suppressive cells are myeloid derived suppressor cells (MDSCs), tumor-associated macrophages (TAMs), neutrophils, T_{reg} cells, and B_{reg} cells. In various embodiments, MDSCs are monocytic MDSCs (M-MDSCs) and polymorphonuclear MDSCs (PMN-MDSCs). In various embodiments, the TAMs are M2 TAMs. In various embodiments, the immune suppressive cells are CAFs. In various embodiments, the levels of immune suppressive cells are reduced by about 5-100% (e.g., reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% compared to one or more blood samples collected from the subject prior to treatment. In various embodiments, the levels of immune suppressive cells are reduced by about 2-100 fold (e.g., about 2, 5, 10, 15, 20, 25, 30, 35, 40, 45, 50, 55, 60, 65, 70, 75, 80, 85, 90, 95, or 100 fold, inclusive of all values and ranges between these values) compared to one or more blood samples collected from the subject prior to treatment. In various embodiments, immune suppressive cells are identified by the assay of cell-surface proteins expression. In various embodiments, administering a RAS inhibitor or combination of compounds described herein to a subject having one or more tumors characterized as immune refractory, immunologically protected, or immunologically “cold” increases the levels of activated pro-inflammatory immune cells by 5-100% (e.g., increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% compared to one or more blood samples collected from the subject prior to treatment. In various embodiments, administering a RAS inhibitor or combination of compounds described herein to a subject having one or more tumors characterized as immune refractory, immunologically protected, or immunologically “cold” increases the levels of activated pro-inflammatory immune cells by 2-100 fold (e.g., about 2, 5, 10, 15, 20, 25, 30, 35, 40, 45, 50, 55, 60, 65, 70, 75, 80, 85, 90, 95, or 100 fold, inclusive of all values and ranges between these values) compared to one or more blood samples collected from the subject prior to treatment. In various embodiments, the activated pro-inflammatory cells are dendritic cells (DCs), macrophages, M1 macrophages, T-cells, B-cells, NK cells, NK-T cells, and iNK cells. In various embodiments, the frequency of pro-inflammatory immune cells is increased to 10-50% (e.g., about 10%, 15%, 20%, 25%, 30%, 35%, 40%, 45%, or 50%, inclusive of all values and ranges between these values) of all leukocytes analyzed from one or more blood samples collected from the subject. In various embodiments, activated pro-inflammatory immune cells are identified by the assay of cell-surface protein expression.

In various embodiments, the analysis of cells in one or more blood samples of a subject suffering from cancer is performed by the assay of cell-surface proteins. In various embodiments, the cell-surface proteins are selected from the group consisting of receptor tyrosine kinase (RTK), CD1c, CD2, CD3, CD4, CD5, CD8, CD9, CD10, CD11b, CD11c, CD14, CD15, CD16, CD18, CD19, CD20, CD21, CD22, CD23, CD24, TACI, CD25, CD27, CD28, CD30, CD30L, CD31, CD32, CD32b, CD34, CD33, CD38, CD39, CD40, CD40-L, CD41b, CD42a, CD42b, CD43, CD44, CD48, CD47, CD45RA, CD45RO, CD48, CD52, CD55, CD56, CD58, CD61, CD66b, CD70, CD72, CD79, CD68, CD84, CD86, CD93, CD94, CD95, CRACC, BLAME, BCMA, CD103, CD107, CD112, CD120a, CD120b, CD123, CD125, CD134, CD135, CD140a, CD141, CD154, CD155, CD160, CD163, CD172a, XCR1, CD203c, CD204, CD206, CD207 CD226, CD244, CD267, CD268, CD269, CD355, CD358, NKG2A, NKG2B, NKG2C, NKG2D, NKG2E, NKG2F, NKG2H, KIR2DL1, KIR2DL2, KIR2DL3, KIR2DL5A, KIR2DL5B, KIR3DL1, KIR3DL2, KIR3DL3, KIR3DL4, KIR2DS1, KIR2DS2, KIR2DS3, KIR2DS4, KIR2DS5, DAP12, KIR3DS, NKp44, NKp46, TCR, BCR, Integrins, FcβεRI, MHC-I, MHC-II, IL-1R, IL-2Rα, IL-2Rβ, IL-2Rγ, IL-3Rα, CSF2RB, IL-4R, IL-5Rα, IL-6Rα, gp130, IL-7Rα, IL-9R, IL-12Rβ1, IL-12Rβ2, IL-13Rα1, IL-13Rα2, IL-15Rα, IL-21R, IL-23R, IL-27Rα, IL-31Rα, OSMR, CSF-1R, cell-surface IL-15, IL-10Rα, IL-10Rβ, IL-20Rα, IL-20Rβ, IL-22Rα1, IL-22Rα2, IL-22Rβ, IL-28RA, PD-1, PD-1H, BTLA, CTLA-4, PD-L1, PD-L2, 2B4, B7-1, B7-2, B7-H1, B7-H4, B7-DC, DR3, LIGHT, LAIR, LTα1β2, LTβR, TIM-1, TIM-3, TIM-4, TIGIT, LAG-3, ICOS, ICOS-L, SLAM, SLAMF2, OX-40, OX-40L, GITR, GITRL, TL1A, HVEM, 41-BB, 41BB-L, TL-1A, TRAF1, TRAF2, TRAF3, TRAF5, BAFF, BAFF-R, APRIL, TRAIL, RANK, AITR, TRAMP, CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6, CXCR7, CLECL9a, DC-SIGN, IGSF4A, SIGLEC, EGFR, PDGFR, VEGFR, FAP, α-SMA, Vimentin, Laminin, FAS, FAS-L, F_{c}, ICAM-1, ICAM-2, ICAM-3, ICAM-4, ICAM-5, PECAM-1, MICA, MICB, UL16, ULBP1, ULBP2, ILBP3, ULBP4, ULBP5, ULBP6, MULT1, RAE1α, β, γ, δ, and ε, A₁R, A_{2A}R, A_{2B}R, and A₃R, H60a, H60b, and H60c. In various embodiments, Integrins are selected from the group consisting of α1, α2, αIIb, α3, α4, α5, α6, α7, α8, α9, α10, α11, αD, αE, αL, αM, αV, αX, β1, β2, β3, β4, β5, β6, β7, β8, or combinations thereof. In various embodiments, TCR is selected from the group consisting of α, β, γ, δ, ε, and ζ TCR. Several methods have been described in the literature for assaying of cell-surface protein expression, including Flow Cytometry and Mass Cytometry (CyTOF). The presence or abundance of one or more of these cell-surface proteins indicates that the patient is responsive to treatment with the method disclosed herein.

In various embodiments, administering a RAS inhibitor or combination of compounds described herein to a subject having one or more tumors characterized as immune refractory, immunologically protected, or immunologically “cold” reduces the neutrophil to lymphocyte (NLR) in one or more blood samples from high to moderate, or high to low. In various embodiments, the analysis of cells from one or more blood samples collected from a subject having one or more tumors characterized as immune refractory, immunologically protected, or immunologically “cold” reduces NLR to between 1-2 (e.g., between 1, 1.1, 1.2, 1.3, 1.4, 1.5, 1.6, 1.7, 1.8, 1.9, and 2 inclusive of all values and ranges between these values). In various embodiments, NLR, after administration of the RAS inhibitor or combination of compounds described herein, is reduced. In various embodiments, NLR, after administration of the RAS inhibitor or combination of compounds described herein is <2.

In various embodiments, administering a RAS inhibitor or combination of compounds described herein to a subject having one or more tumors characterized as immune refractory, immunologically protected, or immunologically “cold” reduces the levels of CTCs in one or blood samples. In various embodiments, the levels of CTCs in blood are reduced to 5, 4, 3, 2, 1, or 0 per 7.5 ml blood inclusive of inclusive of all values and ranges between these values.

In various embodiments, administering a RAS inhibitor or combination of compounds described herein to a subject having one or more tumors characterized as immune refractory, immunologically protected, or immunologically “cold” decreases the levels of tumor promoting, anti-inflammatory, or immune suppressive proteins in one or more blood samples of the subject. In various embodiments, the tumor promoting, anti-inflammatory, or immune suppressive proteins are selected from the group consisting of CD39, CD79, MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27 and MMP28, CXCL12, GM-CSF, G-CSF, TGF-β1, TGF-β2, and TGF-β3, arginase, asparaginase, kyneurinase, indoleamine 2,3 dioxygenase (IDO1 and IDO2), tryptophan 2,3 dioxygenase (TDO), myeloperoxidase (MPO), neutrophil elastase (NE), and IL411. In various embodiments, the levels of tumor promoting, anti-inflammatory, or immune suppressive proteins in one or more blood samples of the subject are decreased by 5-100% (e.g., decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more blood samples collected prior to treatment. In various embodiments, the levels of tumor promoting, anti-inflammatory, or immune suppressive proteins in one or more blood samples of the subject are decreased by 2-100 fold (e.g., decreased relative to one or more samples collected prior to treatment by about 2, 5, 10, 15, 20, 25, 30, 35, 40, 45, 50, 55, 60, 65, 70, 75, 80, 85, 90, 95, or 100 fold, inclusive of all values and ranges between these values) compared to one or more blood samples collected from the subject prior to treatment.

In various embodiments, administering a RAS inhibitor or combination of compounds described herein to a subject having one or more tumors characterized as immune refractory, immunologically protected, or immunologically “cold” increases the levels of tumor inhibiting, anti-tumor, or pro-inflammatory proteins in one or more blood samples collected from the subject. In various embodiments, tumor inhibiting, anti-tumor, or pro-inflammatory proteins are selected from the group consisting of IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-20, IL-21, IL-22, IL-23, IL-24, IL-25, IL-26, IL-27, IL-28, IL-29, IL-30, IL-31, IL-32, IL-33, IL-35, IL-36, cell-surface IL-15, CXCL2 (MCP-1), CXCL3 (MIP-1α), CXCL4 (MIP-1β), CXCL5 (RANTES), IFN-α, IFN-β, IFN-γ, Granzyme-B, Perforin, and TNF-α. In various embodiments, the levels of anti-tumor, or pro-inflammatory proteins are increased by 5-100% (e.g., increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more blood samples collected prior to treatment. In various embodiments, the levels of anti-tumor, or pro-inflammatory proteins are increased by 2-100 fold (e.g., increased relative to one or more samples collected prior to treatment by about 2, 5, 10, 15, 20, 25, 30, 35, 40, 45, 50, 55, 60, 65, 70, 75, 80, 85, 90, 95, or 100 fold, inclusive of all values and ranges between these values) compared to one or more blood samples collected from the subject prior to treatment. Several methods have been described in the literature for assaying proteins from blood samples, including western blot, and ELISA.

In various embodiments, administering a RAS inhibitor or combination of compounds described herein to a subject having one or more tumors characterized as immune refractory, immunologically protected, or immunologically “cold” decreases the levels of neutrophil extracellular traps (NETs) in one or more blood samples collected from the subject. In various embodiments, the levels of NETs in one or more blood samples is decreased by 5-100% (e.g., decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more blood samples collected prior to treatment. In various embodiments, the levels of NETs in one or more blood samples is decreased by 2-100-fold (e.g., by about 2, 5, 10, 15, 20, 25, 30, 35, 40, 45, 50, 55, 60, 65, 70, 75, 80, 85, 90, 95, or 100 fold, inclusive of all values and ranges between these values) compared to one or more blood samples collected from the subject prior to treatment. Several methods have been described in the literature for assaying NETs from blood samples, including western blot, ELISA, and flow cytometry.

In various embodiments, administering a RAS inhibitor or combination of compounds described herein to a subject having one or more tumors characterized as immune refractory, immunologically protected, or immunologically “cold” decreases the expression of tumor promoting, tumor permissive, or immune suppressive genes in one or more blood samples of the subject. In one or more embodiments, the expression of tumor promoting, tumor permissive, or immune suppressive genes is decreased by 5-100% (e.g., decreased relative to levels in one or more blood samples collected prior to treatment by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100%) compared to one or more blood samples collected prior to treatment. In one or more embodiments, the expression of tumor promoting, tumor permissive, or immune suppressive genes is decreased by 2-100-fold (e.g., decreased by about 2, 5, 10, 15, 20, 25, 30, 35, 40, 45, 50, 55, 60,65, 70, 75, 80, 85, 90, 95, or 100 fold, inclusive of all values and ranges between these values) compared to one or more blood samples collected from the subject prior to treatment.

In various embodiments, administering a RAS inhibitor or combination of compounds described herein to a subject having one or more tumors characterized as immune refractory, immunologically protected, or immunologically “cold” increases the expression of tumor inhibiting, anti-tumor, or pro-inflammatory genes in one or more samples collected from the subject. In one or more embodiments, the expression of tumor inhibiting, anti-tumor, or pro-inflammatory genes is increased by 5-100% (e.g., increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more blood samples collected prior to treatment. In various embodiments, the expression of tumor inhibiting, anti-tumor, or pro-inflammatory genes is increased by 2-100-fold (e.g., increased relative to one or more samples collected prior to treatment by about 2, 5, 10, 15, 20, 25, 30, 35, 40, 45, 50, 55, 60, 65, 70, 75, 80, 85, 90, 95, or 100 fold, inclusive of all values and ranges between these values) compared to one or more blood samples collected from the subject prior to treatment. In various embodiments, the gene expression analysis is performed by PCR, RT-PCR, qRT-PCR, next-generation sequencing (NGS), RNA-seq, ATAC-seq, exome sequencing, Southern Blot, microarray analysis, or single-cell sequencing.

In various embodiments, administering a RAS inhibitor or combination of compounds described herein to a subject having one or more tumors characterized as immune refractory, immunologically protected, or immunologically “cold” increases the levels of leukocytes in the tumor. In various embodiments, the levels of leukocytes are increased in the tumor core or tumor periphery. In various embodiments, the leukocytes are increased by 5-100% (e.g., increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% compared to one or more tumor samples collected from the subject prior to treatment. In various embodiments, the levels of leukocytes are increased by 2-100 fold (e.g., about 2, 5, 10, 15, 20, 25, 30, 35, 40, 45, 50, 55, 60, 65, 70, 75, 80, 85, 90, 95, or 100 fold, inclusive of all values and ranges between these values) compared to one or more tumor samples collected from the subject prior to treatment. In various embodiments, the frequency of leukocytes in the tumor core or tumor periphery is ≥5%, ≥10%, ≥15%, ≥20%, ≥25%, ≥30%, ≥35%, ≥40%, ≥45%, or ≥50, inclusive of all values and ranges between these values, of all cells analyzed.

In various embodiments, administering a RAS inhibitor or combination of compounds described herein to a subject having one or more tumors characterized as immune refractory, immunologically protected, or immunologically “cold” reduces the levels of immune suppressive cells in the tumor. In various embodiments, the levels of immune suppressive cells are reduced in the tumor core or tumor periphery. In various embodiments, the suppressive cells are myeloid derived suppressor cells (MDSCs), tumor associated macrophages (TAMs), neutrophils, T_{reg} cells, and B_{reg} cells. In various embodiments, MDSCs are monocytic MDSCs (M-MDSCs) and polymorphonuclear MDSCs (PMN-MDSCs). In various embodiments, the TAMs are M2 TAMs. In various embodiments, the immune suppressive cells are CAFs. In various embodiments, the levels of immune suppressive cells are reduced by about 5-100% (e.g., reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% compared to one or more tumor samples collected from the subject prior to treatment. In various embodiments, the levels of immune suppressive cells are reduced by about 2-100 fold (e.g., about 2, 5, 10, 15, 20, 25, 30, 35,40, 45, 50, 55, 60, 65, 70, 75, 80, 85, 90, 95, or 100 fold, inclusive of all values and ranges between these values) compared to one or more tumor samples collected from the subject prior to treatment. In various embodiments, immune suppressive cells are identified by the assay of cell-surface proteins expression.

Levels of leukocytes in a tumor sample can be evaluated by several methods including flow cytometry and immunohistochemistry. In various embodiments, administering a RAS inhibitor or combination of compounds described herein to a subject having one or more tumors characterized as immune refractory, immunologically protected, or immunologically “cold” increases the levels of activated pro-inflammatory immune cells in the tumor. In various embodiments, the levels of activated pro-inflammatory cells are increased in the tumor core or tumor periphery.

In various embodiments, administering a RAS inhibitor or combination of compounds described herein to a subject having one or more tumors characterized as immune refractory, immunologically protected, or immunologically “cold” increases the levels of activated pro-inflammatory immune cells in the tumor by 5-100% (e.g., increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% compared to one or more tumor samples collected from the subject prior to treatment. In various embodiments, administering a RAS inhibitor or combination of compounds described herein to a subject having one or more tumors characterized as immune refractory, immunologically protected, or immunologically “cold” increases the levels of activated pro-inflammatory immune cells by 2-100 fold (e.g., about 2, 5, 10, 15, 20, 25, 30, 35, 40, 45, 50, 55, 60, 65, 70, 75, 80, 85, 90, 95, or 100 fold, inclusive of all values and ranges between these values) compared to one or more tumor samples collected from the subject prior to treatment. In various embodiments, the activated pro-inflammatory cells are dendritic cells (DCs), macrophages, M1 macrophages, T-cells, B-cells, NK cells, NK-T cells, and NK cells. In various embodiments, the frequency of pro-inflammatory immune cells is between about 10-50% (e.g., about 10%, 15%, 20%, 25%, 30%, 35%, 40%, 45%, or 50%, inclusive of all values and ranges between these values) of all leukocytes analyzed from one or more tumor samples collected from the subject. In various embodiments, activated pro-inflammatory immune cells are identified by the assay of cell-surface protein expression.

In various embodiments, the analysis of cells in one or more tumor samples of a subject suffering from cancer is performed by the assay of cell-surface proteins. In various embodiments, the cell-surface proteins are selected from the group consisting of receptor tyrosine kinase (RTK), CD1c, CD2, CD3, CD4, CD5, CD8, CD9, CD10, CD11b, CD11c, CD14, CD15, CD16, CD18, CD19, CD20, CD21, CD22, CD23, CD24, TACI, CD25, CD27, CD28, CD30, CD30L, CD31, CD32, CD32b, CD34, CD33, CD38, CD39, CD40, CD40-L, CD41b, CD42a, CD42b, CD43, CD44, CD48, CD47, CD45RA, CD45RO, CD48, CD52, CD55, CD56, CD58, CD61, CD66b, CD70, CD72, CD79, CD68, CD84, CD86, CD93, CD94, CD95, CRACC, BLAME, BCMA, CD103, CD107, CD112, CD120a, CD120b, CD123, CD125, CD134, CD135, CD140a, CD141, CD154, CD155, CD160, CD163, CD172a, XCR1, CD203c, CD204, CD206, CD207 CD226, CD244, CD267, CD268, CD269, CD355, CD358, NKG2A, NKG2B, NKG2C, NKG2D, NKG2E, NKG2F, NKG2H, KIR2DL1, KIR2DL2, KIR2DL3, KIR2DL5A, KIR2DL5B, KIR3DL1, KIR3DL2, KIR3DL3, KIR3DL4, KIR2DS1, KIR2DS2, KIR2DS3, KIR2DS4, KIR2DS5, DAP12, KIR3DS, NKp44, NKp46, TCR, BCR, Integrins, FcβεRI, MHC-I, MHC-II, IL-1R, IL-2Rα, IL-2Rβ, IL-2Rγ, IL-3Rα, CSF2Rβ, IL-4R, IL-5Rα, IL-6Rα, gp130, IL-7Rα, IL-9R, IL-12Rβ1, IL-12Rβ2, IL-13Rα1, IL-13Rα2, IL-15Rα, IL-21R, IL-23R, IL-27Rα, IL-31Rα, OSMR, CSF-1R, cell-surface IL-15, IL-10Rα, IL-10Rβ, IL-20Rα, IL-20Rβ, IL-22Rα1, IL-22Rα2, IL-22Rβ, IL-28RA, PD-1, PD-1H, BTLA, CTLA-4, PD-L1, PD-L2, 2B4, B7-1, B7-2, B7-H1, B7-H4, B7-DC, DR3, LIGHT, LAIR, LTα1β2, LTβR, TIM-1, TIM-3, TIM-4, TIGIT, LAG-3, ICOS, ICOS-L, SLAM, SLAMF2, OX-40, OX-40L, GITR, GITRL, TL1A, HVEM, 41-BB, 41BB-L, TL-1A, TRAF1, TRAF2, TRAF3, TRAF5, BAFF, BAFF-R, APRIL, TRAIL, RANK, AITR, TRAMP, CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6, CXCR7, CLECL9a, DC-SIGN, IGSF4A, SIGLEC, EGFR, PDGFR, VEGFR, FAP, a-SMA, Vimentin, Laminin, FAS, FAS-L, Fc, ICAM-1, ICAM-2, ICAM-3, ICAM-4, ICAM-5, PECAM-1, MICA, MICB, UL16, ULBP1, ULBP2, ILBP3, ULBP4, ULBP5, ULBP6, MULT1, RAE1α, β, γ, δ, and ε, A₁R, A_{2A}R, A_{2B}R, and A₃R, H60a, H60b, and H60c. In various embodiments, Integrins are selected from the group consisting of α1, α2, αIIb, α3, α4, α5, α6, α7, α8, α9, α10, α11, αD, αE, αL, αM, αV, αX, β1, β2, β3, β4, β5, β6, β7, β8, or combinations thereof. In various embodiments, TCR is selected from the group consisting of α, β, γ, δ, ε, and ζ TCR. Several methods have been described in the literature for assaying of cell-surface protein expression, including Flow Cytometry and Mass Cytometry (CyTOF). The presence or abundance of one or more of these cell-surface proteins indicates that the patient is responsive to treatment with the method disclosed herein.

In various embodiments, administering a RAS inhibitor or combination of compounds described herein to a subject having one or more tumors characterized as immune refractory, immunologically protected, or immunologically “cold” decreases the levels of tumor promoting, anti-inflammatory, or immune suppressive proteins in one or more tumor samples of the subject. In various embodiments, the tumor promoting, anti-inflammatory, or immune suppressive proteins are selected from the group consisting of CD39, CD79, MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, MMP28, CXCL12, GM-CSF, G-CSF, TGF-β1, TGF-β2, TGF-β3, arginase, asparaginase, kyneurinase, indoleamine 2,3 dioxygenase (IDO1 and IDO2), tryptophan 2,3 dioxygenase (TDO), myeloperoxidase (MPO), neutrophil elastase (NE), and IL4I1. In various embodiments, the levels of tumor promoting, anti-inflammatory, or immune suppressive proteins in one or more tumor samples of the subject are decreased by 5-100% (e.g., decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more tumor samples collected prior to treatment. In various embodiments, the levels of tumor promoting, anti-inflammatory, or immune suppressive proteins in one or more tumor samples of the subject are decreased by 2-100 fold (e.g., decreased relative to one or more samples collected prior to treatment by about 2, 5, 10, 15, 20, 25, 30, 35, 40, 45, 50, 55, 60, 65, 70, 75, 80, 85, 90, 95, or 100 fold, inclusive of all values and ranges between these values) compared to one or more tumor samples collected from the subject prior to treatment.

In various embodiments, administering a RAS inhibitor or combination of compounds described herein to a subject having one or more tumors characterized as immune refractory, immunologically protected, or immunologically “cold” increases the levels of proteins associated with tumor growth inhibition, anti-tumor activity, or pro-inflammatory activity. In various embodiments, proteins associated with tumor growth inhibition, anti-tumor activity, or pro-inflammatory activity are selected from the group consisting of CD44, CD56, CD103c, CD69, KG2A, NKG2B, NKG2C, NKG2D, NKG2E, NKG2F, NKG2H, ICOS, ICOS-L, SLAM, SLAMF2, OX-40, OX-40L, GITR, GITRL, TL-1A, HVEM, 41-BB, 41BB-L, TRAF1, TRAF2, TRAF3, TRAF5, BAFF, BAFF-R, APRIL, TRAIL, RANK, AITR, TRAMP, IL-1α, IL-1β, IL-2, IL-3, IL-4, IL-5, IL-6, IL-7, IL-8, IL-9, IL-10, IL-11, IL-12, IL-13, IL- 14, IL-15, IL-16, IL-17, IL-18, IL-20, IL-21, IL-22, IL-23, IL-24, IL-25, IL-26, IL-27, IL-28, IL-29, IL-30, IL-31, IL-32, IL-33, IL-35, IL-36, CXCL2 (MCP-1), CXCL3 (MIP-1α), CXCL4 (MIP-1β), CXCL5 (RANTES), IFN-α, IFN-β, IFN-γ, Granzyme-B, Perforin, and TNF-α. In various embodiments, the levels of proteins associated with tumor growth inhibition, anti-tumor activity, or pro-inflammatory activity are increased by 5-100% (e.g., increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more tumor samples collected prior to treatment. In various embodiments, the levels of proteins associated with tumor growth inhibition, anti-tumor activity, or pro-inflammatory activity are increased by 2-100 fold (e.g., decreased relative to one or more samples collected prior to treatment by about 2, 5, 10, 15, 20, 25, 30, 35, 40, 45, 50, 55, 60, 65, 70, 75, 80, 85, 90, 95, or 100 fold, inclusive of all values and ranges between these values) compared to one or more tumor samples collected from the subject prior to treatment. Several methods have been described in the literature for assaying proteins from tumor samples, including western blot, and ELISA.

In various embodiments, administering a RAS inhibitor or combination of compounds described herein to a subject having one or more tumors characterized as immune refractory, immunologically protected, or immunologically “cold” decreases the levels of neutrophil extracellular traps (NETs) in one or more tumor samples collected from the subject. In various embodiments, the levels of NETs in one or more tumor samples is decreased by 5-100% (e.g., decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more tumor samples collected prior to treatment. In various embodiments, the levels of NETs in one or more tumor samples is decreased by 2-100-fold (e.g., decreased relative to one or more samples collected prior to treatment by about 2, 5, 10, 15, 20, 25, 30, 35, 40, 45, 50, 55, 60, 65, 70, 75, 80, 85, 90, 95, or 100 fold, inclusive of all values and ranges between these values) compared to one or more tumor samples collected from the subject prior to treatment. Several methods have been described in the literature for assaying NETs from tumor samples, including western blot, ELISA, and flow cytometry.

In various embodiments, administering a RAS inhibitor or combination of compounds described herein to a subject having one or more tumors characterized as immune refractory, immunologically protected, or immunologically “cold” decreases the expression of tumor promoting, tumor permissive, or immune suppressive genes in one or more tumor samples of the subject. In one or more embodiments, the expression of tumor promoting, tumor permissive, or immune suppressive genes is decreased by 5-100% (e.g., decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more tumor samples collected prior to treatment. In one or more embodiments, the expression of tumor promoting, tumor permissive, or immune suppressive genes is decreased by 2-100-fold (e.g., decreased relative to one or more samples collected prior to treatment by about 2, 5, 10, 15, 20, 25, 30, 35, 40, 45, 50, 55, 60, 65, 70, 75, 80, 85, 90, 95, or 100 fold, inclusive of all values and ranges between these values) compared to one or more tumor samples collected from the subject prior to treatment.

In various embodiments, administering a RAS inhibitor or combination of compounds described herein to a subject having one or more tumors characterized as immune refractory, immunologically protected, or immunologically “cold” increases the expression of tumor inhibiting, anti-tumor, or pro-inflammatory genes in one or more samples collected from the subject. In one or more embodiments, the expression of tumor inhibiting, anti-tumor, or pro-inflammatory genes is increased by 5-100% (e.g increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more tumor samples collected prior to treatment. In various embodiments, the expression of tumor inhibiting, anti-tumor, or pro-inflammatory genes is increased by 2-100-fold (e.g., increased by about 2, 5, 10, 15, 20, 25, 30, 35, 40, 45, 50, 55, 60, 65, 70, 75, 80, 85, 90, 95, or 100 fold, inclusive of all values and ranges between these values) compared to one or more tumor samples collected from the subject prior to treatment. In various embodiments, the gene expression analysis is performed by PCR, RT-PCR, qRT-PCR, next-generation sequencing (NGS), RNA-seq, ATAC-seq, exome sequencing, Southern Blot, microarray analysis, or single-cell sequencing.

In various embodiments, treatment of a subject having lung cancer with a RAS inhibitor or combination of compounds described herein switches the cold tumor to a hot tumor. Such switch can be detected using the methods described herein and known in the art. If the subject has been diagnosed with a tumor that has switched from cold to hot tumor, treatment may continue by administering a RAS inhibitor or combination of compounds described herein, wherein the RAS inhibitor or combination of compounds described herein is useful in treating hot tumors, or tumors that are immune cell rich or immunogenic. In other embodiments, when the tumor has switched from cold to hot tumor, the patient stops treatment with a RAS inhibitor or combination of compounds described herein, and the patient begins treatment with a cancer therapeutic that is useful in treating hot tumors, or tumors that are immune cell rich or immunogenic. Such cancer therapeutics include chemotherapeutics, cytokines, angiogenesis inhibitors, enzymes, immune checkpoint modulators and monoclonal antibodies, hormone therapy, comprises one or more cell-based therapies, such as adoptive cell transfer, tumor-infiltrating leukocyte therapy, chimeric antigen receptor T-cell therapy (CAR-T), NK-cell therapy and stem cell therapy, or oncolytic virus or oncolytic bacteria.

In various embodiments, the immune checkpoint modulators target Programmed cell death protein 1 (PD-1), Programmed cell death protein ligand-1 (PD-L1), cytotoxic T-lymphocyte-associated protein 4 (CTLA-4), T-cell Immunoglobulin and mucin-domain containing-3 (TIM-3), Lymphocyte-activation Gene 3 (LAG-3), or TIGIT (T cell immunoreceptor with Ig and ITIM domains). In various embodiments, the immune checkpoint modulator is an antibody selected from the group consisting of ipilimumab, tremelimumab, pembrolizumab, nivolumab, atezolizumab, avelumab, cemiplimab, and durvalumab.

In various embodiments, the subject diagnosed with a cold tumor and receiving therapy with a RAS inhibitor or combination of compounds described herein is monitored regularly to determine if the tumor has switched to a hot tumor. Monitoring may be carried out as determined by a physician to be necessary, e.g., every month, every two months, every three months, every 6 months, or every year.

In various embodiments, the subject has previously been treated with immunotherapy but has developed resistance to immunotherapy or had a shift from a hot tumor to a cold tumor. Also provided is a method of treating a subject having cancer that has developed resistance to immunotherapy or developed a cold tumor comprising administering to the subject a RAS inhibitor or combination of compounds described herein.

### Cancers Having an Oncogenic RAS Mutation

In some embodiments, the invention discloses a method of treating lung cancer in a subject in need thereof, the method comprising administering to the subject a therapeutically effective amount of a compound of the present invention, or a pharmaceutically acceptable salt thereof, or a pharmaceutical composition comprising such a compound or salt, wherein the cancer is an immune refractory lung cancer.

Methods of detecting Ras mutations are known in the art. Such means include, but are not limited to direct sequencing, and utilization of a high-sensitivity diagnostic assay (with CE-IVD mark), e.g., as described in Domagala, et al., Pol J Pathol 3: 145-164 (2012), incorporated herein by reference in its entirety, including TheraScreen PCR; AmoyDx; PNAClamp; RealQuality; EntroGen; LightMix; StripAssay; Hybcell plexA; Devyser; Surveyor; Cobas; and TheraScreen Pyro. See, also, e.g., WO 2020/106640.

In some embodiments, the cancer is non-small cell lung cancer, or any of the lung cancers described herein, and the Ras mutation comprises a K-Ras G12C mutation, an H-Ras G12C mutation, or an N-Ras G12C mutation. In some embodiments, the cancer is non-small cell lung cancer, or any of the lung cancers described herein, and the Ras mutation comprises a K-Ras G12C mutation.

Also provided is a method of inhibiting a Ras protein in a cell, the method comprising contacting the cell with an effective amount of a compound of the present invention, or a pharmaceutically acceptable salt thereof. A method of inhibiting RAF-Ras binding, the method comprising contacting the cell with an effective amount of a compound of the present invention, or a pharmaceutically acceptable salt thereof, is also provided. The cell may be a cancer cell. The cancer cell may be of any type of cancer described herein. The cell may be in vivo or in vitro.

### Lung Cancers

In some embodiments, the invention discloses a method of treating a lung cancer. In some embodiments, the lung cancer is an immune refractory lung cancer.

Lung cancer can be classified using different systems. In one system, lung cancer includes adenocarcinoma (mixed, acinar, papillary, solid, micropapillary, lepidic nonmucinous and lepidic mucinous), squamous cell carcinoma, large cell carcinoma (e.g., non-small cell lung cancers (NSCLC) (e.g., advanced or non-advanced, large cell carcinoma with neuroendocrine morphology (LCNEM), NSCLC—not otherwise specified (NOS)/adenosquamous carcinoma, sarcomatoid carcinoma, adenosquamous carcinoma, and large-cell neuroendocrine carcinoma (LCNEC)); and small cell lung cancer/carcinoma (SCLC)).

Alternatively, in a different system, lung cancer can be classified into preinvasive lesions, minimally invasive adenocarcinoma, and invasive adenocarcinoma (invasive mucinous adenocarcinoma, mucinous bronchioloalveolar carcinoma (BAC), colloid, fetal (low and high grade), and enteric). Non-small cell lung cancer comprises adenocarcinoma, squamous cell carcinoma, large cell carcinoma, or large cell neuroendocrine tumors.

More frequently, lung cancer may be categorized as either small cell lung cancer (“SCLC”) or non-small cell lung cancer (“NSCLC”). NSCLCs may be further categorized as squamous or non-squamous. An example of a non-squamous NSCLC is adenocarcinoma.

In some embodiments, lung cancer is bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, or mesothelioma.

The lung cancer may be newly diagnosed and naïve to treatment, or may be relapsed, refractory, relapsed and refractory, locally advanced, or metastatic. In some instances, the lung cancer comprises a relapsed or refractory lung cancer. In some instances, the lung cancer comprises a metastatic lung cancer. In some cases, the subject is diagnosed with a relapsed or refractory lung cancer. In additional cases, the subject is diagnosed with a metastatic lung cancer.

### Combination Therapies

The methods of the invention may include a compound of the invention used alone or in combination with one or more additional therapies (e.g., non-drug treatments or therapeutic agents). The dosages of one or more of the additional therapies (e.g., non-drug treatments or therapeutic agents) may be reduced from standard dosages when administered alone. For example, doses may be determined empirically from drug combinations and permutations or may be deduced by isobolographic analysis (e.g., Black et al., Neurology 65:S3-S6 (2005)).

A compound of the present invention may be administered before, after, or concurrently with one or more of such additional therapies. When combined, dosages of a compound of the invention and dosages of the one or more additional therapies (e.g., non-drug treatment or therapeutic agent) provide a therapeutic effect (e.g., synergistic or additive therapeutic effect). A compound of the present invention and an additional therapy, such as an anti-cancer agent, may be administered together, such as in a unitary pharmaceutical composition, or separately and, when administered separately, this may occur simultaneously or sequentially. Such sequential administration may be close or remote in time.

In some embodiments, the additional therapy is the administration of side-effect limiting agents (e.g., agents intended to lessen the occurrence or severity of side effects of treatment. For example, in some embodiments, the compounds of the present invention can also be used in combination with a therapeutic agent that treats nausea. Examples of agents that can be used to treat nausea include: dronabinol, granisetron, metoclopramide, ondansetron, and prochlorperazine, or pharmaceutically acceptable salts thereof.

In some embodiments, the one or more additional therapies includes a non-drug treatment (e.g., surgery or radiation therapy). In some embodiments, the one or more additional therapies includes a therapeutic agent (e.g., a compound or biologic that is an anti-angiogenic agent, signal transduction inhibitor, antiproliferative agent, glycolysis inhibitor, or autophagy inhibitor). In some embodiments, the one or more additional therapies includes a non-drug treatment (e.g., surgery or radiation therapy) and a therapeutic agent (e.g., a compound or biologic that is an anti-angiogenic agent, signal transduction inhibitor, antiproliferative agent, glycolysis inhibitor, or autophagy inhibitor). In other embodiments, the one or more additional therapies includes two therapeutic agents. In still other embodiments, the one or more additional therapies includes three therapeutic agents. In some embodiments, the one or more additional therapies includes four or more therapeutic agents.

In this Combination Therapy section, all references are incorporated by reference for the agents described, whether explicitly stated as such or not.

### Non-Drug Therapies

Examples of non-drug treatments include, but are not limited to, radiation therapy, cryotherapy, hyperthermia, surgery (e.g., surgical excision of tumor tissue), and T cell adoptive transfer (ACT) therapy.

In some embodiments, the compounds of the invention may be used as an adjuvant therapy after surgery. In some embodiments, the compounds of the invention may be used as a neo-adjuvant therapy prior to surgery.

Radiation therapy may be used for inhibiting abnormal cell growth or treating a hyperproliferative disorder, such as cancer, in a subject (e.g., mammal (e.g., human)). Techniques for administering radiation therapy are known in the art. Radiation therapy can be administered through one of several methods, or a combination of methods, including, without limitation, external-beam therapy, internal radiation therapy, implant radiation, stereotactic radiosurgery, systemic radiation therapy, radiotherapy, and permanent or temporary interstitial brachy therapy. The term “brachy therapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present invention include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, or Y-90. Moreover, the radionuclide(s) can be embodied in a gel or radioactive micro spheres.

In some embodiments, the compounds of the present invention can render abnormal cells more sensitive to treatment with radiation for purposes of killing or inhibiting the growth of such cells. Accordingly, this invention further relates to a method for sensitizing abnormal cells in a mammal to treatment with radiation which comprises administering to the mammal an amount of a compound of the present invention, which amount is effective to sensitize abnormal cells to treatment with radiation. The amount of the compound in this method can be determined according to the means for ascertaining effective amounts of such compounds described herein. In some embodiments, the compounds of the present invention may be used as an adjuvant therapy after radiation therapy or as a neo-adjuvant therapy prior to radiation therapy.

In some embodiments, the non-drug treatment is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 7,572,631; 5,883,223; 6,905,874; 6,797,514; and 6,867,041.

### Therapeutic Agents

A therapeutic agent may be a compound used in the treatment of cancer or symptoms associated therewith.

For example, a therapeutic agent may be a steroid. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, fiucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy with a compound of the present invention include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO01/37820, WO01/32651, WO02/68406, WO02/66470, WO02/55501, WO04/05279, WO04/07481, WO04/07458, WO04/09784, WO02/59110, WO99/45009, WO00/59509, WO99/61422, WO00/12089, and WO00/02871.

A therapeutic agent may be a biologic (e.g., cytokine (e.g., interferon or an interleukin such as IL-2)) used in treatment of cancer or symptoms associated therewith. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

A therapeutic agent may be a T-cell checkpoint inhibitor. In one embodiment, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, that interacts with a checkpoint protein. In some embodiments, the checkpoint inhibitor is an agent, such as an antibody, that interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PD-L1. In some embodiments, the checkpoint inhibitor is an inhibitor or antagonist (e.g., an inhibitory antibody or Fc fusion or small molecule inhibitor) of PD-L2 (e.g., a PD-L2/lg fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the checkpoint inhibitor is pembrolizumab, nivolumab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A317 (BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514/MED10680, BMS936559, MED14736, MPDL3280A, MSB0010718C, BMS986016, IMP321, lirilumab, IPH2101, 1-7F9, and KW-6002.

A therapeutic agent may be an anti-TIGIT antibody, such as MBSA43, BMS-986207, MK-7684, COM902, AB154, MTIG7192A or OMP-313M32 (etigilimab).

A therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents.

Anti-cancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Further anti-cancer agents include leucovorin (LV), irenotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18:233a (1999), and Douillard et al., Lancet 355(9209):1041-1047 (2000).

Other non-limiting examples of anti-cancer agents include Gleevec® (Imatinib Mesylate); Kyprolis® (carfilzomib); Velcade® (bortezomib); Casodex (bicalutamide); Iressa® (gefitinib); alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gammaII and calicheamicin omegaII (see, e.g., Agnew, Chem. Intl. Ed Engl. 33:183-186 (1994)); dynemicin such as dynemicin A; bisphosphonates such as clodronate; an esperamicin; neocarzinostatin chromophore and related chromoprotein enediyne antiobiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., Taxol® (paclitaxel), Abraxane® (cremophor-free, albumin-engineered nanoparticle formulation of paclitaxel), and Taxotere® (doxetaxel); chloranbucil; tamoxifen (Nolvadex™); raloxifene; aromatase inhibiting 4(5)-imidazoles; 4-hydroxytamoxifen; trioxifene; keoxifene; LY 117018; onapristone; toremifene (Fareston®); flutarnide, nilutamide, bicalutamide, leuprolide, goserelin; chlorambucil: Gemzar® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; Navelbine® (vinorelbine); novantrone; teniposide; edatrexate; daunomycin; aminopterin; ibandronate; irinotecan (e.g., CPT-11); topoisomerase inhibitor RFS 2000; difluoromethylornithine (DMFO); retinoids such as retinoic acid; esperamicins; capecitabine (e.g., Xeloda®); and pharmaceutically acceptable salts of any of the above.

Additional non-limiting examples of anti-cancer agents include trastuzumab (Herceptin®), bevacizumab (Avastin®), cetuximab (Erbitux®), rituximab (Rituxan®), Taxol®, Arimidex®, ABVD, avicine, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, laniquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexaamethylmelaamine and thiotepa), CDK inhibitors (e.g., a CDK4/6 inhibitor such as abemaciclib, ribociclib, palbociclib; seliciclib, UCN-01, P1446A-05, PD-0332991, dinaciclib, P27-00, AT-7519, RGB286638, and SCH727965), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, histone deacetylase (HDAC) inhibitors (e.g., trichostatin, sodium butyrate, apicidan, suberoyl anilide hydroamic acid, vorinostat, LBH 589, romidepsin, ACY-1215, and panobinostat), mTOR inhibitors (e.g., vistusertib, temsirolimus, everolimus, ridaforolimus, and sirolimus), KSP(Eg5) inhibitors (e.g., Array 520), DNA binding agents (e.g., Zalypsis®), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TG02 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), aurora kinase inhibitors (e.g., MLN8237), cell surface monoclonal antibodies (e.g., anti-CD38 (HUMAX-CD38), anti-CSI (e.g., elotuzumab), HSP90 inhibitors (e.g., 17 AAG and KOS 953), P13K/Akt inhibitors (e.g., perifosine), Akt inhibitors (e.g., GSK-2141795), PKC inhibitors (e.g., enzastaurin), FTIs (e.g., Zarnestra™), anti-CD138 (e.g., BT062), TorcI/2 specific kinase inhibitors (e.g., INK128), ER/UPR targeting agents (e.g., MKC-3946), cFMS inhibitors (e.g., ARRY-382), JAK1/2 inhibitors (e.g., CYT387), PARP inhibitors (e.g., olaparib and veliparib (ABT-888)), and BCL-2 antagonists.

In some embodiments, an anti-cancer agent is selected from mechlorethamine, camptothecin, ifosfamide, tamoxifen, raloxifene, gemcitabine, Navelbine®, sorafenib, or any analog or derivative variant of the foregoing.

In some embodiments, the anti-cancer agent is a HER2 inhibitor. Non-limiting examples of HER2 inhibitors include monoclonal antibodies such as trastuzumab (Herceptin®) and pertuzumab (Perjeta®); small molecule tyrosine kinase inhibitors such as gefitinib (Iressa®), erlotinib (Tarceva®), pilitinib, CP-654577, CP-724714, canertinib (CI 1033), HKI-272, lapatinib (GW-572016; Tykerb®), PKI-166, AEE788, BMS-599626, HKI-357, BIBW 2992, ARRY-334543, and JNJ-26483327.

In some embodiments, an anti-cancer agent is an ALK inhibitor. Non-limiting examples of ALK inhibitors include ceritinib, TAE-684 (NVP-TAE694), PF02341066 (crizotinib or 1066), alectinib; brigatinib; entrectinib; ensartinib (X-396); lorlatinib; ASP3026; CEP-37440; 4SC-203; TL-398; PLB1003; TSR-011; CT-707; TPX-0005, and AP26113. Additional examples of ALK kinase inhibitors are described in examples 3-39 of WO05016894.

In some embodiments, an anti-cancer agent is an inhibitor of a member downstream of a Receptor Tyrosine Kinase (RTK)/Growth Factor Receptor (e.g., a SHP2 inhibitor (e.g., SHP099, TNO155, RMC-4550, RMC-4630, JAB-3068, JAB-3312, RLY-1971, ERAS-601, SH3809, PF-07284892, or BBP-398), an SOS1 inhibitor (e.g., BI-1701963, BI-3406, SDR5, or BAY-293), a Raf inhibitor, a MEK inhibitor, an ERK inhibitor, a PI3K inhibitor, a PTEN inhibitor, an AKT inhibitor, or an mTOR inhibitor (e.g., mTORC1 inhibitor or mTORC2 inhibitor). In some embodiments, the anti-cancer agent is JAB-3312.

In some embodiments, an anti-cancer agent is an additional Ras inhibitor or a Ras vaccine, or another therapeutic modality designed to directly or indirectly decrease the oncogenic activity of Ras. In some embodiments, an anti-cancer agent is an additional Ras inhibitor. In some embodiments, the Ras inhibitor targets Ras in its active, or GTP-bound state. In some embodiments, the Ras inhibitor targets Ras in its inactive, or GDP-bound state. In some embodiments, the Ras inhibitor is, such as an inhibitor of K-Ras G12C, such as AMG 510, MRTX1257, MRTX849, JNJ-74699157, LY3499446, or ARS-1620, ARS-853, BPI-421286, LY3537982, JDQ443, AZ4625, JAB-21000, or GDC-6036. In some embodiments, the Ras inhibitor is an inhibitor of K-Ras G12D, such as MRTX1133 or JAB-22000. In some embodiments, the Ras inhibitor is a K-Ras G12V inhibitor, such as JAB-23000. In some embodiments, the Ras inhibitor is RMC-6236. In some embodiments, the Ras inhibitor is selected from a Ras(ON) inhibitor (that is, Ras in its GTP-bound state) disclosed in the following, incorporated herein by reference in their entireties, or a pharmaceutically acceptable salt, solvate, isomer (e.g., stereoisomer), prodrug, or tautomer thereof: WO 2021091982, WO 2021091967, WO 2021091956 and WO 2020132597. Other examples of Ras inhibitors that may be combined with a Ras inhibitor of the present invention are provided in the following, incorporated herein by reference in their entireties: WO 2022002102, WO 2022002018, WO 2021259331, WO 2021257828, WO 2021252339, WO 2021248095, WO 2021248090, WO 2021248083, WO 2021248082, WO 2021248079, WO 2021248055, WO 2021245051, WO 2021244603, WO 2021239058, WO 2021231526, WO 2021228161, WO 2021219090, WO 2021219090, WO 2021219072, WO 2021218939, WO 2021217019, WO 2021216770, WO 2021215545, WO 2021215544, WO 2021211864, WO 2021190467, WO 2021185233, WO 2021180181, WO 2021175199, 2021173923, WO 2021169990, WO 2021169963, WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018217651, WO 2018218071, WO 2018218069, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659 and WO 2013155223.

In some embodiments, a therapeutic agent that may be combined with a compound of the present invention is an inhibitor of the MAP kinase (MAPK) pathway (or “MAPK inhibitor”). MAPK inhibitors include, but are not limited to, one or more MAPK inhibitor described in Cancers (Basel) 2015 September; 7(3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, RO4987655 (CH4987655); CI-1040; PD-0325901; CH5126766; MAP855; AZD6244; refametinib (RDEA 119/BAY 86-9766); GDC-0973/XL581; AZD8330 (ARRY-424704/ARRY-704); RO5126766 (Roche, described in PLoS One. 2014 Nov. 25; 9(11)); and GSK1120212 (or JTP-74057, described in Clin Cancer Res. 2011 Mar. 1; 17(5):989-1000). The MAPK inhibitor may be PLX8394, LXH254, GDC-5573, or LY3009120.

In some embodiments, an anti-cancer agent is a disrupter or inhibitor of the RAS-RAF-ERK or PI3K-AKT-TOR or PI3K-AKT signaling pathways. The PI3K/AKT inhibitor may include, but is not limited to, one or more PI3K/AKT inhibitor described in Cancers (Basel) 2015 September; 7(3): 1758-1784. For example, the PI3K/AKT inhibitor may be selected from one or more of NVP-BEZ235; BGT226; XL765/SAR245409; SF1126; GDC-0980; PI-103; PF-04691502; PKI-587; GSK2126458.

In some embodiments, an anti-cancer agent is a PD-1 or PD-L1 antagonist.

In some embodiments, additional therapeutic agents include ALK inhibitors, HER2 inhibitors, EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune therapies, such as an immune checkpoint inhibitor. In some embodiments, a therapeutic agent may be a pan-RTK inhibitor, such as afatinib.

IGF-1R inhibitors include linsitinib, or a pharmaceutically acceptable salt thereof.

EGFR inhibitors include, but are not limited to, small molecule antagonists, antibody inhibitors, or specific antisense nucleotide or siRNA. Useful antibody inhibitors of EGFR include cetuximab (Erbitux®), panitumumab (Vectibix®), zalutumumab, nimotuzumab, and matuzumab. Further antibody-based EGFR inhibitors include any anti-EGFR antibody or antibody fragment that can partially or completely block EGFR activation by its natural ligand. Non-limiting examples of antibody-based EGFR inhibitors include those described in Modjtahedi et al., Br. J. Cancer 1993, 67:247-253; Teramoto et al., Cancer 1996, 77:639-645; Goldstein et al., Clin. Cancer Res. 1995, 1:1311-1318; Huang et al., 1999, Cancer Res. 15:59(8):1935-40; and Yang et al., Cancer Res. 1999, 59:1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

Small molecule antagonists of EGFR include gefitinib (Iressa®), erlotinib (Tarceva®), and lapatinib (TykerB®). See, e.g., Yan et al., Pharmacogenetics and Pharmacogenomics In Oncology Therapeutic Antibody Development, BioTechniques 2005, 39(4):565-8; and Paez et al., EGFR Mutations In Lung Cancer Correlation With Clinical Response To Gefitinib Therapy, Science 2004, 304(5676):1497-500. In some embodiments, the EGFR inhibitor is osimertinib (Tagrisso®). Further non-limiting examples of small molecule EGFR inhibitors include any of the EGFR inhibitors described in the following patent publications, and all pharmaceutically acceptable salts of such EGFR inhibitors: EP 0520722; EP 0566226; WO96/33980; U.S. Pat. No. 5,747,498; WO96/30347; EP 0787772; WO97/30034; WO97/30044; WO97/38994; WO97/49688; EP 837063; WO98/02434; WO97/38983; WO95/19774; WO95/19970; WO97/13771; WO98/02437; WO98/02438; WO97/32881; DE 19629652; WO98/33798; WO97/32880; WO97/32880; EP 682027; WO97/02266; WO97/27199; WO98/07726; WO97/34895; WO96/31510; WO98/14449; WO98/14450; WO98/14451; WO95/09847; WO97/19065; WO98/17662; U.S. Pat. Nos. 5,789,427; 5,650,415; 5,656,643; WO99/35146; WO99/35132; WO99/07701; and WO92/20642. Additional non-limiting examples of small molecule EGFR inhibitors include any of the EGFR inhibitors described in Traxler et al., Exp. Opin. Ther. Patents 1998, 8(12):1599-1625. In some embodiments, an EGFR inhibitor is an ERBB inhibitor. In humans, the ERBB family contains HER1 (EGFR, ERBB1), HER2 (NEU, ERBB2), HER3 (ERBB3), and HER (ERBB4).

MEK inhibitors include, but are not limited to, pimasertib, selumetinib, cobimetinib (Cotellic®), trametinib (Mekinist®), and binimetinib (Mektovi®). In some embodiments, a MEK inhibitor targets a MEK mutation that is a Class I MEK1 mutation selected from D67N; P124L; P124S; and L177V. In some embodiments, the MEK mutation is a Class II MEK1 mutation selected from ΔE51-Q58; ΔF53-Q58; E203K; L177M; C121S; F53L; K57E; Q56P; and K57N.

PI3K inhibitors include, but are not limited to, wortmannin; 17-hydroxywortmannin analogs described in WO06/044453; 4-[2-(1H-Indazol-4-yl)-6-[[4-(methylsulfonyl)piperazin-1-yl]methyl]thieno[3,2-d]pyrimidin-4-yl]morpholine (also known as pictilisib or GDC-0941 and described in WO09/036082 and WO09/055730); 2-methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in WO06/122806); (S)—I-(4-((2-(2-aminopyrimidin-5-yl)-7-methyl-4-morpholinothieno[3,2-d]pyrimidin-6-yl)methyl)piperazin-1-yl)-2-hydroxypropan-1-one (described in WO08/070740); LY294002 (2-(4-morpholinyl)-8-phenyl-4H-1-benzopyran-4-one (available from Axon Medchem); PI 103 hydrochloride (3-[4-(4-morpholinylpyrido-[3′,2′:4,5]furo[3,2-d]pyrimidin-2-yl]phenol hydrochloride (available from Axon Medchem); PIK 75 (2-methyl-5-nitro-2-[(6-bromoimidazo[1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo[1,2-c]quinazolin-5-yl)-nicotinamide (available from Axon Medchem); AS-252424 (5-[1-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino)ethyl]-4H-pyrido-[1,2-a]pyrimidin-4-one (available from Axon Medchem); XL-765; and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136.

AKT inhibitors include, but are not limited to, Akt-1-1 (inhibits Aktl) (Barnett et al., Biochem. J. 2005, 385(Pt. 2): 399-408); Akt-1-1,2 (inhibits Akl and 2) (Barnett et al., Biochem. J. 2005, 385(Pt. 2): 399-408); API-59CJ-Ome (e.g., Jin et al., Br. J. Cancer 2004, 91:1808-12); 1-H-imidazo[4,5-c]pyridinyl compounds (e.g., WO 05/011700); indole-3-carbinol and derivatives thereof (e.g., U.S. Pat. No. 6,656,963; Sarkar and Li J Nutr. 2004, 134(12 Suppl):3493S-3498S); perifosine (e.g., interferes with Akt membrane localization; Dasmahapatra et al. Clin. Cancer Res. 2004, 10(15):5242-52); phosphatidylinositol ether lipid analogues (e.g., Gills and Dennis Expert. Opin. Investig. Drugs 2004, 13:787-97); and triciribine (TCN or API-2 or NCI identifier: NSC 154020; Yang et al., Cancer Res. 2004, 64:4394-9).

mTOR inhibitors include, but are not limited to, ATP-competitive mTORC1/mTORC2 inhibitors, e.g., PI-103, PP242, PP30; Torin 1; FKBP12 enhancers; 4H-1-benzopyran-4-one derivatives; and rapamycin (also known as sirolimus) and derivatives thereof, including: temsirolimus (Torisel®); everolimus (Afinitor®; WO94/09010); ridaforolimus (also known as deforolimus or AP23573); rapalogs, e.g., as disclosed in WO98/02441 and WO01/14387, e.g. AP23464 and AP23841; 40-(2-hydroxyethyl)rapamycin; 40-[3-hydroxy(hydroxymethyl)methylpropanoate]-rapamycin (also known as CC1779); 40-epi-(tetrazolyt)-rapamycin (also called ABT578); 32-deoxorapamycin; 16-pentynyloxy-32(S)-dihydrorapanycin; derivatives disclosed in WO05/005434; derivatives disclosed in U.S. Pat. Nos. 5,258,389, 5,118,677, 5,118,678, 5,100,883, 5,151,413, 5,120,842, and 5,256,790, and in WO94/090101, WO92/05179, WO93/111130, WO94/02136, WO94/02485, WO95/14023, WO94/02136, WO95/16691, WO96/41807, WO96/41807, and WO2018204416; and phosphorus-containing rapamycin derivatives (e.g., WO05/016252). In some embodiments, the mTOR inhibitor is a bisteric inhibitor (see, e.g., WO2018204416, WO2019212990 and WO2019212991), such as RMC-5552, having the structure

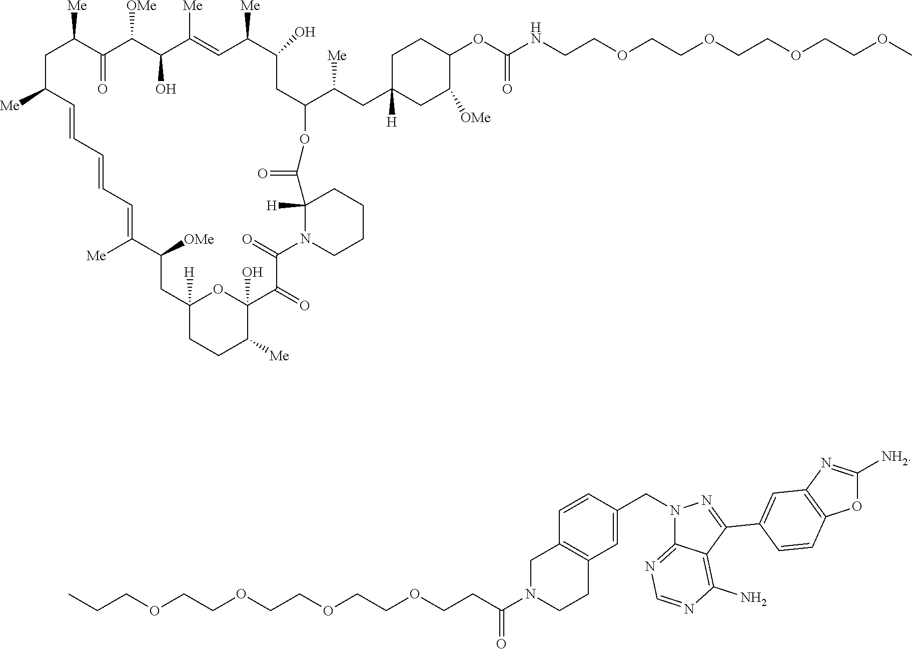

BRAF inhibitors that may be used in combination with compounds of the invention include, for example, vemurafenib, dabrafenib, and encorafenib. A BRAF may comprise a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N581I; D594N; D594G; D594A; D594H; F595L; G596D; G596R and A762E.

MCL-1 inhibitors include, but are not limited to, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263.

In some embodiments, the additional therapeutic agent is selected from the group consisting of a MEK inhibitor, a HER2 inhibitor, a SHP2 inhibitor, a CDK4/6 inhibitor, an mTOR inhibitor, a SOS1 inhibitor, and a PD-L1 inhibitor. In some embodiments, the additional therapeutic agent is selected from the group consisting of a MEK inhibitor, a SHP2 inhibitor, and a PD-L1 inhibitor. See, e.g., Hallin et al., Cancer Discovery, DOI: 10.1158/2159-8290 (Oct. 28, 2019) and Canon et al., Nature, 575:217 (2019). In some embodiments, a Ras inhibitor of the present invention is used in combination with a MEK inhibitor and a SOS1 inhibitor. In some embodiments, a Ras inhibitor of the present invention is used in combination with a PD-L1 inhibitor and a SOS1 inhibitor. In some embodiments, a Ras inhibitor of the present invention is used in combination with a PD-L1 inhibitor and a SHP2 inhibitor. In some embodiments, a Ras inhibitor of the present invention is used in combination with a MEK inhibitor and a SHP2 inhibitor. In some embodiments, the cancer is colorectal cancer and the treatment comprises administration of a Ras inhibitor of the present invention in combination with a second or third therapeutic agent.

Proteasome inhibitors include, but are not limited to, carfilzomib (Kyprolis®), bortezomib (Velcade®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PD-L1, anti-CTLA4, anti-LAGI, and anti-OX40 agents).

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al., Blood 2007, 110(1):186-192; Thompson et al., Clin. Cancer Res. 2007, 13(6):1757-1761; and WO06/121168 A1), as well as described elsewhere herein.

GITR agonists include, but are not limited to, GITR fusion proteins and anti-GITR antibodies (e.g., bivalent anti-GITR antibodies), such as, a GITR fusion protein described in U.S. Pat. Nos. 6,111,090, 8,586,023, WO2010/003118 and WO2011/090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO2011/028683, WO2013/039954, WO05/007190, WO07/133822, WO05/055808, WO99/40196, WO01/03720, WO99/20758, WO06/083289, WO05/115451, and WO2011/051726.

Another example of a therapeutic agent that may be used in combination with the compounds of the invention is an anti-angiogenic agent. Anti-angiogenic agents are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloprotienase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO96/33172, WO96/27583, WO98/07697, WO98/03516, WO98/34918, WO98/34915, WO98/33768, WO98/30566, WO90/05719, WO99/52910, WO99/52889, WO99/29667, WO99007675, EP0606046, EP0780386, EP1786785, EP1181017, EP0818442, EP1004578, and US20090012085, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF (e.g., bevacizumab), or soluble VEGF receptors or a ligand binding region thereof) such as VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as Vectibix® (panitumumab), erlotinib (Tarceva®), anti-Ang1 and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US2003/0162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 2002/0042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol (EntreMed, USA); TLC ELL-12 (Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5beta3 integrin, second generation (Applied Molecular Evolution, USA and MedImmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-Ialfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT 510 (Abbott, USA); AAL 993 (Novartis, Switzerland); VEGI (ProteomTech, USA); tumor necrosis factor-alpha inhibitors; SU 11248 (Pfizer, USA and SUGEN USA); ABT 518, (Abbott, USA); YH16 (Yantai Rongchang, China); S-3APG (Boston Childrens Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A4 prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577 (Aventis, France); WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists(ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with compounds of the invention include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met.

Another example of a therapeutic agent that may be used in combination with compounds of the invention is an autophagy inhibitor. Autophagy inhibitors include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used. In some embodiments, the one or more additional therapies include an autophagy inhibitor.

Another example of a therapeutic agent that may be used in combination with compounds of the invention is an anti-neoplastic agent. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-NI, interferon alfa-n3, interferon alfacon-1, interferon alpha, natural, interferon beta, interferon beta-Ia, interferon beta-Ib, interferon gamma, natural interferon gamma-Ia, interferon gamma-Ib, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE 941 (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with compounds of the invention include ipilimumab (Yervoy®); tremelimumab; galiximab; nivolumab, also known as BMS-936558 (Opdivo®); pembrolizumab (Keytruda®); avelumab (Bavencio®); AMP224; BMS-936559; MPDL3280A, also known as RG7446; MEDI-570; AMG557; MGA271; IMP321; BMS-663513; PF-05082566; CDX-1127; anti-OX40 (Providence Health Services); huMAbOX40L; atacicept; CP-870893; lucatumumab; dacetuzumab; muromonab-CD3; ipilumumab; MEDI4736 (Imfinzi®); MSB0010718C; AMP 224; adalimumab (Humira®); ado-trastuzumab emtansine (Kadcyla®); aflibercept (Eylea®); alemtuzumab (Campath®); basiliximab (Simulect®); belimumab (Benlysta®); basiliximab (Simulect®); belimumab (Benlysta®); brentuximab vedotin (Adcetris®); canakinumab (Ilaris®); certolizumab pegol (Cimzia®); daclizumab (Zenapax®); daratumumab (Darzalex®); denosumab (Prolia®); eculizumab (Soliris®); efalizumab (Raptiva®); gemtuzumab ozogamicin (Mylotarg®); golimumab (Simponi®); ibritumomab tiuxetan (Zevalin®); infliximab (Remicade®); motavizumab (Numax®); natalizumab (Tysabri®); obinutuzumab (Gazyva®); ofatumumab (Arzerra®); omalizumab (Xolair®); palivizumab (Synagis®); pertuzumab (Perjeta®); pertuzumab (Perjeta®); ranibizumab (Lucentis®); raxibacumab (Abthrax®); tocilizumab (Actemra®); tositumomab; tositumomab-i-131; tositumomab and tositumomab-i-131 (Bexxar®); ustekinumab (Stelara®); AMG 102; AMG 386; AMG 479; AMG 655; AMG 706; AMG 745; and AMG 951.

The compounds described herein can be used in combination with the agents disclosed herein or other suitable agents, depending on the condition being treated. Hence, in some embodiments the one or more compounds of the disclosure will be co-administered with other therapies as described herein. When used in combination therapy, the compounds described herein may be administered with the second agent simultaneously or separately. This administration in combination can include simultaneous administration of the two agents in the same dosage form, simultaneous administration in separate dosage forms, and separate administration. That is, a compound described herein and any of the agents described herein can be formulated together in the same dosage form and administered simultaneously. Alternatively, a compound of the invention and any of the therapies described herein can be simultaneously administered, wherein both the agents are present in separate formulations. In another alternative, a compound of the present disclosure can be administered and followed by any of the therapies described herein, or vice versa. In some embodiments of the separate administration protocol, a compound of the invention and any of the therapies described herein are administered a few minutes apart, or a few hours apart, or a few days apart.

In some embodiments of any of the methods described herein, the first therapy (e.g., a compound of the invention) and one or more additional therapies are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours, up to 24 hours, or up to 1-7, 1-14, 1-21 or 1-30 days before or after the one or more additional therapies.

The invention also features kits including (a) a pharmaceutical composition including an agent (e.g., a compound of the invention) described herein, and (b) a package insert with instructions to perform any of the methods described herein. In some embodiments, the kit includes (a) a pharmaceutical composition including an agent (e.g., a compound of the invention) described herein, (b) one or more additional therapies (e.g., non-drug treatment or therapeutic agent), and (c) a package insert with instructions to perform any of the methods described herein. As one aspect of the present invention contemplates the treatment of the disease or symptoms associated therewith with a combination of pharmaceutically active compounds that may be administered separately, the invention further relates to combining separate pharmaceutical compositions in kit form. The kit may comprise two separate pharmaceutical compositions: a compound of the present invention, and one or more additional therapies. The kit may comprise a container for containing the separate compositions such as a divided bottle or a divided foil packet. Additional examples of containers include syringes, boxes, and bags. In some embodiments, the kit may comprise directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

### Pharmaceutical Compositions

The disclosure provides pharmaceutical compositions including one or more RAS inhibitor compounds, or a pharmaceutically acceptable salt thereof, and a pharmaceutically acceptable excipient.

In some embodiments, a compound is present in a pharmaceutical composition in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Compounds described herein, whether expressly stated or not, may be provided or utilized in salt form, e.g., a pharmaceutically acceptable salt form, unless expressly stated to the contrary.

The compounds of the disclosure may have ionizable groups so as to be capable of preparation as pharmaceutically acceptable salts. These salts may be acid addition salts involving inorganic or organic acids or the salts may, in the case of acidic forms of the compounds of the disclosure, be prepared from inorganic or organic bases. In some embodiments, the compounds are prepared or used as pharmaceutically acceptable salts prepared as addition products of pharmaceutically acceptable acids or bases. Suitable pharmaceutically acceptable acids and bases are well-known in the art, such as hydrochloric, sulfuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-optionally substituted hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

For use as treatment of subjects, the compounds of the disclosure, or a pharmaceutically acceptable salt thereof, can be formulated as pharmaceutical or veterinary compositions. Depending on the subject to be treated, the mode of administration, and the type of treatment desired, e.g., prevention, prophylaxis, or therapy, the compounds, or a pharmaceutically acceptable salt thereof, are formulated in ways consonant with these parameters. A summary of such techniques may be found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compositions can be prepared according to conventional mixing, granulating or coating methods, respectively, and the present pharmaceutical compositions can contain from about 0.1% to about 99%, from about 5% to about 90%, or from about 1% to about 20% of a compound of the present disclosure, or pharmaceutically acceptable salt thereof, by weight or volume. In some embodiments, compounds, or a pharmaceutically acceptable salt thereof, described herein may be present in amounts totaling 1-95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the disclosure, or a pharmaceutically acceptable salt thereof. Suitable forms include syrups, capsules, and tablets, as is understood in the art.

Each compound, or a pharmaceutically acceptable salt thereof, as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating the compound, or a pharmaceutically acceptable salt thereof, into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, or halogenated fluorocarbon.

The liquid forms in which the compounds, or a pharmaceutically acceptable salt thereof, and compositions of the present disclosure can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the disclosure, or a pharmaceutically acceptable salt thereof, will depend on the nature of the compound, and can readily be determined by one skilled in the art. A dosage may be, for example, about 0.001 mg to about 2000 mg per day, about 1 mg to about 1000 mg per day, about 5 mg to about 500 mg per day, about 100 mg to about 1500 mg per day, about 500 mg to about 1500 mg per day, about 500 mg to about 2000 mg per day, or any range derivable therein.

In some embodiments, the pharmaceutical composition may further include an additional compound having antiproliferative (e.g., anti-cancer) activity. Depending on the mode of administration, compounds, or a pharmaceutically acceptable salt thereof, will be formulated into suitable compositions to permit facile delivery. Each compound, or a pharmaceutically acceptable salt thereof, of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

It will be appreciated that the compounds and pharmaceutical compositions of the present disclosure can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics or procedures and the desired therapeutic effect to be achieved. It will also be appreciated that the therapies employed may achieve a desired effect for the same disorder, or they may achieve different effects (e.g., control of any adverse effects).

Administration of each drug in a combination therapy, as described herein, can, independently, be one to four times daily for one day to one year, and may even be for the life of the subject. Chronic, long-term administration may be indicated.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

Relevant to the examples, persons of skill in the art will recognize that the parental LL2 model is characterized in the literature as a ‘cold’ tumor model. See, e.g., world wide web at drugdevelopment.labcorp.com/industry-solutions/oncology/preclinical/tumor-spotlights/II-2-an-immunosuppressive-murine-tumor-model.html (dated October 2019, last visited Mar. 7, 2022).

In the Examples described herein, Compound A is Compound BA 647 of Table B1. Compound B is Compound DA 122 inhibitor of Table DIa.

### Example 1. The Combination of Compound A with Compound B or SHP2 Potentiated T Cell Function and Improved Anti-Tumor Activity, with the Triple Combination Translating into Additional Durable Complete Responses on Treatment

Methods: Effects of Compound A and combination therapy with Compound B and/or RMC-4550 (a SHP2 inhibitor) on tumor cell growth in vivo were evaluated in the murine syngeneic eLL2 KRAS^{WT/G12C} NRAS⁻/− A2 model using female C57BL/6J mice (6-8 weeks old). Mice were implanted with tumor cells (3×10⁶ cells/mouse) in DMEM medium without supplements subcutaneously in the upper right flank. Once tumors reached an average size of ˜130 mm³, mice were randomized to treatment groups to start the administration of test articles or vehicle. Compound A was administered by oral gavage once daily (po qd) at 200 mg/kg, compound B was administered by oral gavage once daily (po qd) at 25 mg/kg and RMC-4550 was administered by oral gavage once daily (po qd) at 30 mg/kg. In the dual combinations the compounds were administered at the same dose as the monotherapies qd. In the triple combination Compound A was dosed at 200 mg/kg po qd, Compound B was dosed at 50 mg/kg po q2d and RMC-4550 was dosed at 20 mg/kg po qd. The administration of Compound A, Compound B and RMC-4550 was ceased after 60 days of treatment in the monotherapy and combination groups. Body weight and tumor volume (using calipers) was measured twice weekly until study endpoints.

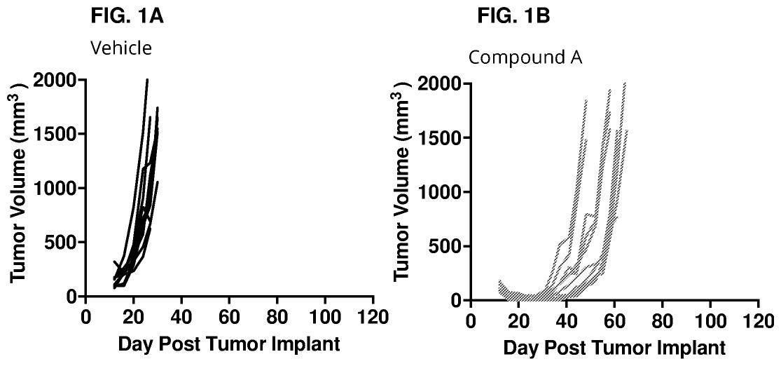

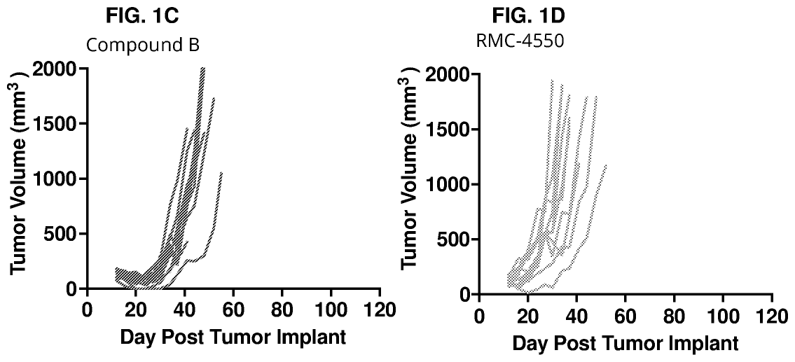

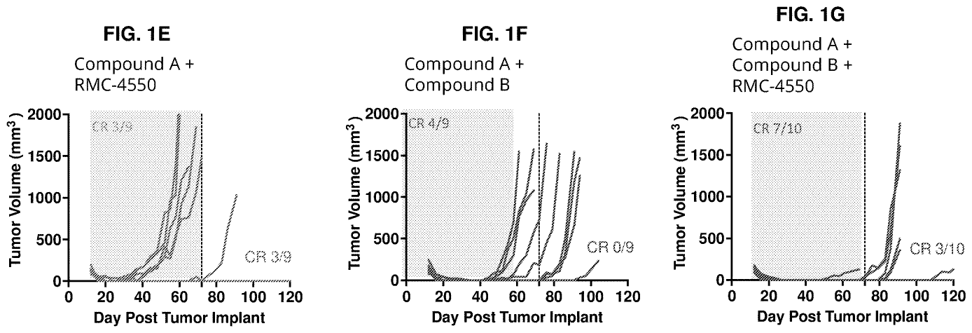

Results: Single-agent Compound A achieved 70% transient complete regressions, Compound B achieved 20% transient complete regressions, RMC-4550 achieved tumor growth inhibition, the combination of Compound A with RMC-4550 achieved 87.5% transient complete regressions and the combination of Compound A with compound B as well as the triple combination. At the treatment stop (day 72) all tumors treated with monotherapies started regrowing, the Compound A and RMC-4550 combination showed 33.3% complete responses, the combination of Compound A and Compound B showed 44.4% complete responses and the triple combination showed 70% complete responses. At the endpoint of the study the Compound A and RMC-4550 combination achieved 33.3% durable complete responses, Compound A in combination with Compound B did not achieve durable complete responses and the triple combination achieved 30% complete responses. These data are depicted in FIGS. 1A-1G

### Example 2. The Combination of Compound A with Compound B or SHP2 Potentiated T Cell Function and Improved Anti-Tumor Activity, with the Triple Combination Translating into Additional Durable Complete Responses on Treatment

Methods: The tumor immune profile of the murine syngeneic eLL2 KRAS^{WT/G12C} NRAS^{-/-} A2 tumors (3 animals/group) was evaluated in response to therapy by flow cytometry. The mono- and combination therapies were administered for 4 days at the concentrations described in FIG. 1 A-C. 24 hours post last dose the tumors were collected, tumor tissue was minced, processed with the Dri Tumor & Tissue Dissociation Reagent from BD Biosciences, and homogenized with the gentleMACS™ Dissociator. The cell suspension was incubated at 4° C. for 30 minutes with Mouse BD Fc Block (Clone 2.4G2 from BD Pharmingen), 10 minutes with Blue Dead Cell Stain Kit (from Invitrogen) and 30 min in cell staining buffer. Antibodies used targeted CD45 (Clone 30-F11 from BD Biosciences), CD19 (Clone 1D3 from BD Biosciences, CD3ε (Clone 145-2C11 from Biolegend), CD8b (Clone H35-17.2 from BD Biosciences), CD4 (Clone GK1.5 from Biolegend), CD11b (Clone M1/70 from Biolegend), Ly-6G (Clone 1A8 from BD Biosciences) and Ly-6C (Clone HK1.4 from Biolegend). For intracellular staining cells were resuspended in 100 ul RPMI+CD107a (1:500)+Golgi Stop/Plug (1:500) and incubated at 37 C for 4 hours. For cell surface staining the cell suspensions were incubated at 4° C. for 30 minutes with Mouse BD Fc Block (Clone 2.4G2 from BD Pharmingen), 10 minutes with Blue Dead Cell Stain Kit (from Invitrogen) and 30 min in cell staining buffer. Cells were permeabilized and fixed for 30 minutes with the eBioscience™ Intracellular Fixation & Permeabilization Buffer Set. The cells were incubated for 1 hour at room temperature with the intracellular IFNg (Clone XMG1.2 from Biolegend) antibody. The Aurora flow spectrometer from Cytek was used to collect and analyze the samples.

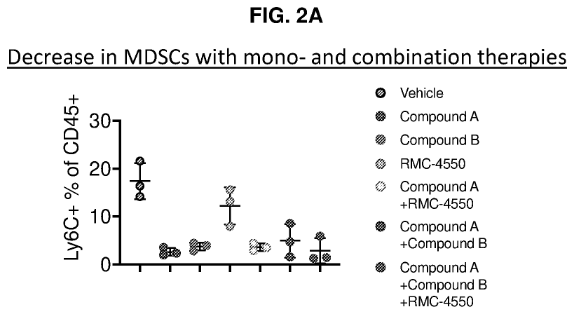

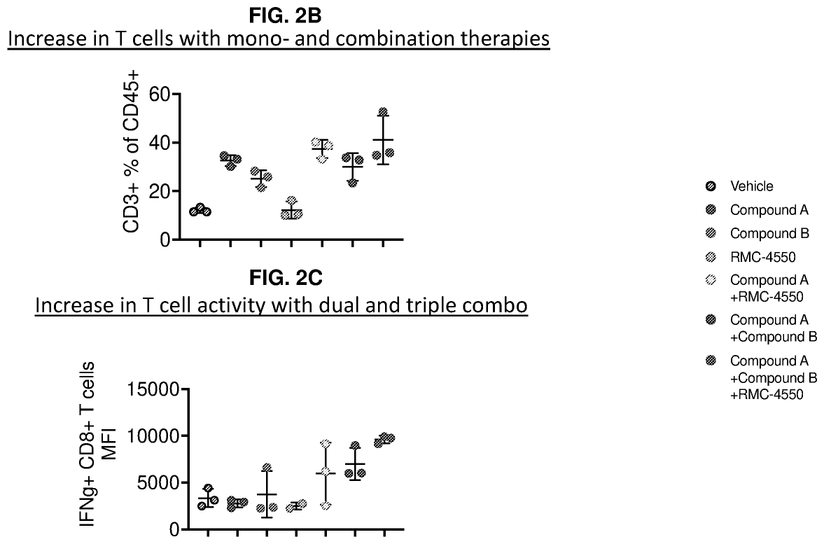

Results: Treatment with Compound A, Compound B, the dual combination of Compound A and RMC-4550, Compound A and Compound B or the triple combination results in almost complete decrease on Ly6C+ myeloid derived suppressor cells and a significant increase in T cells. The monotherapy with RMC-4550 shows only a slight decrease in the immune-suppressive population. While the increase in T cells is only slightly elevated in the combination groups compared to control, there is a marked increase in T cell function, characterized by IFNg secretion. These data are depicted in FIGS. 2A-2C

### OTHER EMBODIMENTS

While the disclosure has been described in connection with specific embodiments thereof, it will be understood that it is capable of further modifications and this application is intended to cover any variations, uses, or adaptations of the disclosure following, in general, the principles of the disclosure and including such departures from the disclosure that come within known or customary practice within the art to which the disclosure pertains and may be applied to the essential features hereinbefore set forth, and follows in the scope of the claims. Other embodiments are within the claims.

1. A method of treating cancer in a subject, the method comprising administering to the subject
a) a first RAS inhibitor, or a pharmaceutically acceptable salt thereof;
b) a second RAS inhibitor, or a pharmaceutically acceptable salt thereof; and
c) a SHP2 inhibitor, or a pharmaceutically acceptable salt thereof.

2. The method of claim 1, wherein the cancer is an immune refractory cancer.

3. The method of claim 1 or 2, wherein the subject has previously been administered an immune checkpoint inhibitor.

4. The method of any one of claims 1 to 3, wherein the subject is resistant to treatment with an immune checkpoint inhibitor.

5. The method of claim 4, wherein the subject has acquired resistance to treatment with an immune checkpoint inhibitor.

6. The method of any one of claims 1 to 5, wherein the administering sensitizes the cancer to treatment with an immune checkpoint inhibitor.

7. The method of any one of claims 1 to 6, further comprising administering to the subject an immune checkpoint inhibitor.

8. The method of claim 7, wherein the immune checkpoint inhibitor is a PD-1 inhibitor.

9. The method of any one of claims 1 to 8, wherein the inhibitors are administered simultaneously or sequentially.

10. The method of any one of claims 1 to 9, wherein the inhibitors are administered as a single formulation or in separate formulations.

11. The method of any one of claims 1 to 10, wherein the subject has one or more tumors with a low tumor mutational burden.

12. The method of any one of claims 1 to 11, wherein the subject has one or more microsatellite stable tumors.

13. The method of any one of claims 1 to 12, wherein the subject has one or more tumors with low microsatellite instability.

14. The method of any one of claims 1 to 13, wherein the subject has one or more tumors with a low tumor immune infiltrate.

15. The method of any one of claims 1 to 14, wherein the administering alters the tumor immune infiltrate.

16. The method of claim 14 or 15, wherein the tumor immune infiltrate comprises antigen-presenting cells, myeloid cells, or lymphoid cells.

17. The method of any one of claims 1 to 16, wherein the administering alters the anti-tumor immune response.

18. The method of any one of claims 1 to 17, wherein the administering alters the tumor microenvironment.

19. The method of any one of claims 1 to 18, wherein the administering transforms an immunologically cold tumor into an immunologically hot tumor.

20. The method of any one of claims 1 to 19, wherein the method reduces tumor size or inhibits tumor growth.

21. The method of any one of claims 1 to 20, wherein the first RAS inhibitor is a RAS G12C inhibitor.

22. The method of any one of claims 1 to 21, wherein the second RAS inhibitor is a RAS(MULTI) inhibitor.

23. The method of any one of claims 1 to 22, wherein the first RAS inhibitor is a RAS G12C inhibitor and the second RAS inhibitor is RAS(MULTI) inhibitor.

24. The method of any one of claims 1 to 23, wherein the SHP2 inhibitor is RMC-4550, or a pharmaceutically acceptable salt thereof.

25. The method of any one of claims 1 to 23, wherein the SHP2 inhibitor is RMC-4630, or a pharmaceutically acceptable salt thereof.

26. The method of any one of claims 1 to 25, wherein the second RAS inhibitor has the structure of Formula AI: 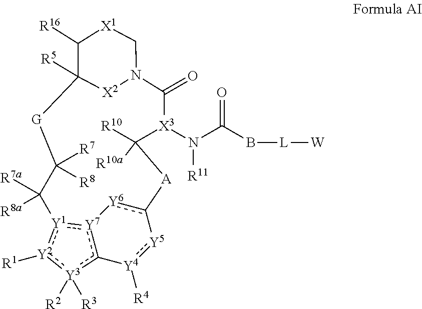
wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is hydrogen, cyano, S(O)₂R′, optionally substituted amino, optionally substituted amido, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R¹⁶ is hydrogen or C₁-C₃ alkyl.

27. The method of any one of claims 1 to 26, wherein the second RAS inhibitor is a compound selected from Table A1 or Table A2.

28. The method of any one of claims 1 to 27, wherein the first RAS inhibitor has the structure of Formula BI: 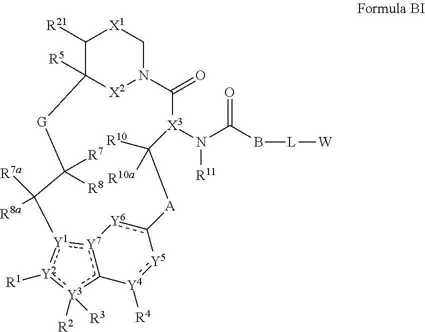
wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′} C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl; or
R⁹ and R^{9′}, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R²¹ is hydrogen or C₁-C₃ alkyl.

29. The compound of any one of claims 1 to 28, wherein the first RAS inhibitor is a compound selected from Table B1 or Table B2.

30. The compound of any one of claims 1 to 25, 28, and 29, wherein the second RAS inhibitor is a compound having the structure of Formula DIa: 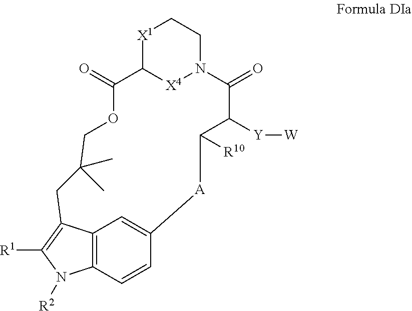
wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene; 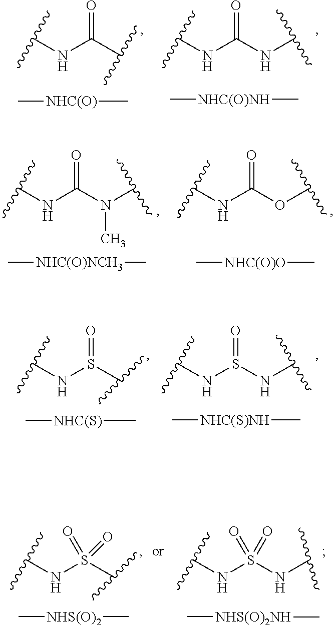
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X¹ and X⁴ are each, independently, CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and R¹⁰ is hydrogen, hydroxy, optionally substituted C₁-C₃ alkyl, or optionally substituted C₁-C₆ heteroalkyl.

31. The compound of any one of claims 1 to 30, wherein the second RAS inhibitor is a compound selected from Table DIa, Table DIb, Table D2, or Table D3

32. The method of any one of claims 1-31, wherein the cancer is a lung cancer.

33. The method of claim 32, wherein the lung cancer is an immune refractory lung cancer.

34. The method of claim 33, wherein the immune refractory lung cancer is non-small cell lung cancer or small-cell lung cancer.

35. The method of claim 33 or 34, wherein the immune refractory lung cancer comprises a Ras mutation.

36. The method of claim 35, wherein the Ras mutation is K-Ras G12C, H-Ras G12C, or N-Ras G12C.

37. The method of claim 36, wherein the Ras mutation is K-Ras G12C.

38. Use of a first RAS inhibitor, or a pharmaceutically acceptable salt thereof; a second RAS inhibitor, or a pharmaceutically acceptable salt thereof; and a SHP2 inhibitor, or a pharmaceutically acceptable salt thereof, in the manufacture of a medicament for treating cancer.

39. A composition comprising a first RAS inhibitor, or a pharmaceutically acceptable salt thereof; a second RAS inhibitor, or a pharmaceutically acceptable salt thereof; and a SHP2 inhibitor, or a pharmaceutically acceptable salt thereof, for use in a method of treating cancer in a subject in need thereof.
